(19) United States
(12) Patent Application Publication
(10) Pub. No.: US 2026/0042755 A1
(43) Pub. Date: Feb. 12, 2026

(54) GPR6 INVERSE AGONISTS

(71) Applicant: MapLight Therapeutics, Inc., Redwood City, CA (US)

(72) Inventors: Andrew GOOD, Redwood City, CA (US); Michael WOOD, Gloucester, MA (US); Vinod F. PATEL, Acton, MA (US); Mark SYLVESTER, Groton, MA (US); Alexandre LARIVEE, Redwood City, CA (US); Tarek MOHAMED, Redwood City, CA (US); Maksym DIACHENKO, Redwood City, CA (US); David Fabian LEON-RAYO, Redwood City, CA (US); Amol Pandharianath JADHAV, Redwood City, CA (US); Shekhar Reddy DURUPALA, Redwood City, CA (US); Lea CONSTANTINEAU-FORGET, Redwood City, CA (US)

(21) Appl. No.: 19/102,150

(22) PCT Filed: Aug. 10, 2023
(86) PCT No.: PCT/US2023/072046

§ 371 Date: Feb. 7, 2025

Related U.S. Application Data
(60) Provisional application No. 63/397,064, filed on Aug. 11, 2022.
(60) Provisional application No. 63/433,956, filed on Dec. 20, 2022.

(51) Int. Cl. C07D 471/04; C07D 403/14; C07D 487/04; C07D 498/14; C07D 519/00

(52) U.S. Cl. CPC C07D 471/04; C07D 403/14; C07D 487/04; C07D 498/14; C07D 519/00

(57) ABSTRACT

Provided herein, is a compound of Formula (I) or a pharmaceutically acceptable salt thereof, wherein Cy, Hy, X, Y, Rₐ, R_{b}, R_{c}, and R_{d} are defined herein as well as compositions and methods of use thereof.

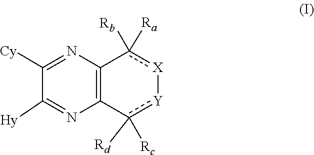

### CROSS-REFERENCE TO RELATED APPLICATIONS

This application claims priority to U.S. Provisional Application No. 63/397,064, filed Aug. 11, 2022, and U.S. Provisional Application No. 63/433,956, filed Dec. 20, 2023, the disclosure of each of which is incorporated by reference in its entirety for all purposes.

### BACKGROUND

The role of the striatum in controlling motor function (Prager 2019) and in modulating reward (Biez-Mendoza 2013) has been recognized for years. Recently, there is a greater appreciation for the role of the striatum in learning as well (Graybiel 2015). The orphan G-protein coupled receptor GPR6 is densely expressed in medium spiny neurons (MSNs) which constitute 95% of the neurons in the mammalian striatum (Kreitzer 2011). GPR6 is expressed in both of the major subtypes (i.e., direct-pathway and indirect-pathway) of MSNs and it is believed to modulate reactivity of the striatum to incoming stimuli. GPR6 is a member of a receptor family that includes orphan receptors GPR3 & GPR12. All three members exhibit high constitutive activity as evidenced by the production of cyclic AMP (cAMP) upon over-expression. Reversal of constitutive GPR6 activity in the human striatum may treat deficits in reward, learning and motor control. There is a need for novel GPR6 inverse agonists.

### SUMMARY OF THE DISCLOSURE

The present disclosure is directed to compounds that are inverse agonists of the G protein-coupled receptor 6 (GPR6) as well as pharmaceutical compositions and uses thereof in treating a disease or disorder that is treatable by administration of a GPR6 inverse agonist.

In one aspect, the present disclosure provides compound of Formula (I), or a pharmaceutically acceptable salt thereof:

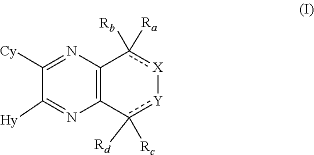

wherein
Rₐ, R_{b}, R_{c}, and R_{d} are each independently absent, H, alkyl, alkenyl, halogen, —CN, —CH₂NRₑR_{f}, —NRₑR_{f}, —NRₑC(═O)R_{f}, —NRₑC(═O)NR_{f}R_{g}, —NRₑC(═O)OR_{f}, —NRₑSO₂R_{f}, —OC(═O)NRₑR_{f}, —ORₑ, —SRₑ, —SORₑ, —SO₂Rₑ, —SO₂ORₑ, —SO₂NRₑR_{f}, —C(═O)Rₑ, —C(═O)ORₑ, —C(═O)C(CH₃)₂OH, —C(═O)NRₑR_{f}, spirocyclyl, spiroheterocyclyl, carbocyclyl, or heterocyclyl; Rₐ and R_{b} together with the atoms to which they are attached form ═NSO₂Rₑ, ═O, carbocyclyl, or heterocyclyl; R_{c} and R_{d} together with the atoms to which they are attached form ═NSO₂Rₑ, ═O, carbocyclyl, or heterocyclyl; or Rₐ and R_{c} together with the atoms to which they are attached form a carbocyclyl or heterocyclyl; Rₐ and Y together with the atoms to which they are attached form a carbocyclyl or heterocyclyl; R_{c} and X together with the atoms to which they are attached form a carbocyclyl or heterocyclyl;
each of X and Y is independently O, N, NRₑ, N(C(Rₑ)₂C(═O)NHR_{f}), N(C(═O)Rₑ), N(C(═O)ORₑ), NC(═O)C(Rₑ)₃, NC(═O)C(Rₑ)₂(Rₑ)₃, S, S(═O), S(═O)₂, S(═O)(═NRₑ), S(═NRₑ)₂, C(CN), C(CN)Rₑ, C(C(═O)Rₑ), C(Rₕ), NS(O)₂NRₑRₕ, NS(O)NRₑRₕ, C(═NSO₂Rₑ), or C(Rₕ)₂; provided that X and Y cannot both be selected from C(CN), C(CN)Rₑ, C(C(═O)Rₑ) and C(Rₕ);
each Rₑ, R_{f}, and R_{g} is independently H, halogen, —OH, —CN, —OCHF₂, —C(CH₃)₂OH, -haloalkyl, —CH₂OH, CH(CH₃)CN, —C((CH₃)₂CN), C(═O)CH₃, —NH₂, NH(alkyl), —CH₂CH₂OCH₃, —CH₂CH(OH)CH₂OH, alkoxy, alkyl, carbocyclyl, or heterocyclyl,
each Rₕ is independently H, halogen, OH, —CN, alkyl, —C(Rₑ)₃, —C(Rₑ)₂C(Rₑ)₃, —CH₂C(Rₑ)₂C(Rₑ)₃, C(═O)C(Rₑ)₃, —OCH₂C(Rₑ)₃, —NHCH₂C(Rₑ)₃, —N(alkyl)CH₂C(Rₑ)₃, —O-heterocyclyl, alkoxy, haloalkoxy, —NRₑR_{f}, —NRₑC(═O)R_{f}, —NRₑC(═O)NR_{f}R_{g}, —CH₂NRₑC(═O)NR_{f}R_{g}, —CH₂C(═O)NRₑR_{f}, —NRₑC(═O)ORₑ, —NRₑSO₂R_{f}, —OC(═O)NRₑR_{f}, —ORₑ, —SRₑ, —SORₑ, —SO₂Rₑ, —SO₂ORₑ, —SO₂NRₑR_{f}, —C(═O)Rₑ, —C(═O)ORₑ, —C(═O)NRₑR_{f}, carbocyclyl, aryl or heterocyclyl; or two Rₕ attached to the same carbon atom together with the atoms to which they are attached form ═NSO₂Rₑ, ═O, carbocyclyl, heteroaryl or heterocyclyl;
Hy is a carbon-linked 3-, 4-, 5- or 6-membered carbocyclyl; a carbon-linked 3-10-membered monocyclic heterocyclyl; or a carbon-linked 3-10-membered bicyclic heterocyclyl; and
Cy is -A-L-B or -A=L-B;
wherein
A is a nitrogen-containing heterocyclylene linked by a nitrogen atom to

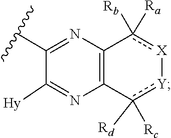

B is a 5- to 7-membered heterocyclyl or a 5- to 7-membered carbocyclyl; and
L is absent, C(Rₕ), C(Rₕ)₂, OC(Rₕ)₂, C(═O), C(═NSO₂Rₑ), C(═O)N(Rₑ), NRₑ, O, S, N, S(═O), S(═O)₂, S(═O)(═NRₑ), or S(═NRₑ)₂, wherein when L is absent or NRₑ, B is aryl or heteroaryl.

In one aspect, the present disclosure provides a compound of Formula (I), or a pharmaceutically acceptable salt thereof:

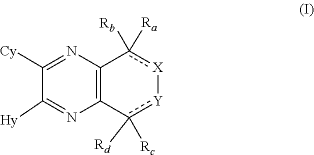

wherein
Rₐ, R_{b}, R_{c}, and R_{d} are each independently absent, H, alkyl, halogen, —CN, —CH₂NRₑR_{f}, —NRₑR_{f}, —NRₑC(═O)R_{f}, —NRₑC(═O)NR_{f}R_{g}, —NRₑC(═O)OR_{f}, —NRₑSO₂R_{f}, —OC(═O)NRₑR_{f}, —ORₑ, —SRₑ, —SORₑ, —SO₂Rₑ, —SO₂ORₑ, —SO₂NRₑR_{f}, —C(═O)Rₑ, —C(═O)ORₑ, —C(═O)NRₑR_{f}, (hetero)alkyl, (hetero)alkenyl, (hetero)alkynyl, spirocyclyl, spiroheterocyclyl, carbocyclyl, or heterocyclyl; Rₐ and R_{b} together with the atoms to which they are attached form ═NSO₂Rₑ, ═O, carbocyclyl, or heterocyclyl; R_{c} and R_{d} together with the atoms to which they are attached form ═NSO₂Rₑ, ═O, carbocyclyl, or heterocyclyl; or Rₐ and R_{c} together with the atoms to which they are attached form a carbocyclyl or heterocyclyl; Rₐ and Y together with the atoms to which they are attached form a carbocyclyl or heterocyclyl; R_{c} and X together with the atoms to which they are attached form a carbocyclyl or heterocyclyl; each of X and Y is independently selected from the group consisting of O, N, NRₑ, N(C(Rₑ)₂C(═O)NHR_{f}), N(C(═O)Rₑ), N(C(═O)ORₑ), NC(═O)C(Rₑ)₃, S, S(═O), S(═O)₂, S(═O)(═NRₑ), S(═NRₑ)₂, NS(═O)₂Rₑ, C(CN), C(CN)Rₑ, C(C(═O)Rₑ), C(Rₕ), NS(O)₂NRₑRₕ, NS(O)NRₑRₕ, C(═NSO₂Rₑ), and C(Rₕ)₂;
each Rₑ, R_{f}, and R_{g} is independently H, —CN, —OCHF₂, alkoxy, alkyl, (hetero)alkyl, (hetero)alkenyl, (hetero)alkynyl, carbocyclyl, or heterocyclyl; Hy is a carbon-linked 3-, 4-, 5- or 6-membered carbocyclic ring or a carbon-linked 3-, 4-, 5- or 6-membered heterocyclic ring comprising at least one N; and
Cy is -A-L-B;
wherein
A is a saturated 4- to 7-membered hetero- or carbocyclic ring, a saturated spirocyclic or spiroheterocyclic ring;
B is a 5- to 7-membered hetero- or carbocyclic ring; and
L is absent, C(Rₕ)₂, C(═O), C(═NSO₂Rₑ), NRₑ, O, S, N, S(═O), S(═O)₂, S(═O)(═NRₑ), or S(═NRₑ)₂, wherein when L is absent or NRₑ, B is aryl or heteroaryl,
wherein
each Rₕ is independently H, halogen, —CN, —OCHF₂, alkyl, alkoxy, —NRₑR_{f}, —NRₑC(═O)R_{f}, —NRₑC(═O)NR_{f}R_{g}, —NRₑC(═O)OR_{f}, —NRₑSO₂R_{f}, —OC(═O)NRₑR_{f}, —ORₑ, —SRₑ, —SORₑ, —SO₂Rₑ, —SO₂ORₑ, —SO₂NRₑR_{f}, —C(═O)Rₑ, —C(═O)ORₑ, —C(═O)NRₑR_{f}, (hetero)alkyl, (hetero)alkenyl, (hetero)alkynyl, carbocyclyl, or heterocyclyl; or two Rₕ attached to the same carbon atom together with the atoms to which they are attached form ═NSO₂Rₑ, ═O, carbocyclyl, or heterocyclyl.

In one aspect, the present disclosure provides a compound of Formula (I′), or a pharmaceutically acceptable salt thereof:

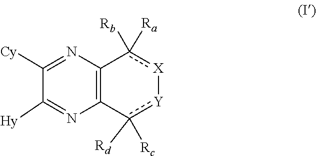

wherein
Rₐ, R_{b}, R_{c}, and R_{d} are each independently absent, H, alkyl, halogen, —CN, —CH₂NRₑR_{f}, —NRₑR_{f}, —NRₑC(═O)R_{f}, —NRₑC(═O)NR_{f}R_{g}, —NRₑC(═O)OR_{f}, —NRₑSO₂R_{f}, —OC(═O)NRₑR_{f}, —ORₑ, —SRₑ, —SORₑ, —SO₂Rₑ, —SO₂ORₑ, —SO₂NRₑR_{f}, —C(═O)Rₑ, —C(═O)ORₑ, —C(═O)NRₑR_{f}, (hetero)alkyl, (hetero)alkenyl, (hetero)alkynyl, spirocyclyl, spiroheterocyclyl, carbocyclyl, or heterocyclyl; Rₐ and R_{b} together with the atoms to which they are attached form ═NSO₂Rₑ, ═O, carbocyclyl, or heterocyclyl; R_{c} and R_{d} together with the atoms to which they are attached form ═NSO₂Rₑ, ═O, carbocyclyl, or heterocyclyl; or Rₐ and R_{c} together with the atoms to which they are attached form a carbocyclyl or heterocyclyl; Rₐ and Y together with the atoms to which they are attached form a carbocyclyl or heterocyclyl; R_{c} and X together with the atoms to which they are attached form a carbocyclyl or heterocyclyl; each of X and Y is independently selected from the group consisting of O, N, NRₑ, N(C(Rₑ)₂C(═O)NHR_{f}), N(C(═O)Rₑ), N(C(═O)ORₑ), NC(═O)C(Rₑ)₃, S, S(═O), S(═O)₂, S(═O)(═NRₑ), S(═NRₑ)₂, NS(═O)₂Rₑ, C(CN), C(CN)Rₑ, C(C(═O)Rₑ), C(Rₕ), NS(O)₂NRₑRₕ, NS(O)NRₑRₕ, C(═NSO₂Rₑ), and C(Rₕ)₂, wherein at least one of X and Y is O, N, NRₑ, N(C(Rₑ)₂C(═O)NHR_{f}), N(C(═O)Rₑ), N(C(═O)ORₑ), NC(═O)C(Rₑ)₃, S, S(═O), S(═O)₂, S(═O)(═NRₑ), S(═NRₑ)₂, NS(═O)₂Rₑ, NS(O)₂NRₑRₕ, or NS(O)NRₑRₕ;
each Rₑ, R_{f}, and R_{g} is independently H, —OH, —CN, —OCHF₂, —CF₃, —CH₂OH, CH(CH₃)CN, —C((CH₃)₂CN), —NHCH₃, —N(CH₃)₂, —CH₂CH(OH)CH₂OH, ═O, alkoxy, alkyl, (hetero)alkyl, (hetero)alkenyl, (hetero)alkynyl, carbocyclyl, or heterocyclyl;
Hy is a carbon-linked 3-, 4-, 5- or 6-membered carbocyclic ring or a carbon-linked 3-, 4-, 5-, or 6-membered heterocyclic ring comprising one or more S or O; and
Cy is -A-L-B;
wherein
A is a piperidine or piperazine ring;
B is a 5- to 7-membered hetero- or carbocyclic ring; and
L is absent, C(Rₕ)₂, C(═O), C(═NSO₂Rₑ), NRₑ, O, S, N, S(═O), S(═O)₂, S(═O)(═NRₑ), or S(═NRₑ)₂, wherein when L is absent or NRₑ, B is aryl or heteroaryl,
wherein
each Rₕ is independently H, halogen, —CN, —OCHF₂, alkyl, alkoxy, —CH₂OH, —CH(OH)C(Rₑ)₃, —CH(OH)CRₑ, —NRₑR_{f}, —NRₑC(═O)R_{f}, —NRₑC(═O)NR_{f}R_{g}, —NRₑC(═O)OR_{f}, —NRₑSO₂R_{f}, —OC(═O)NRₑR_{f}, —ORₑ, —SRₑ, —SORₑ, —SO₂Rₑ, —SO₂ORₑ, —SO₂NRₑR_{f}, —C(═O)Rₑ, —C(═O)ORₑ, —C(═O)NRₑR_{f}, (hetero)alkyl, (hetero)alkenyl, (hetero)alkynyl, carbocyclyl, or heterocyclyl; or two Rₕ attached to the same carbon atom together with the atoms to which they are attached form ═NSO₂Rₑ, ═O, carbocyclyl, or heterocyclyl.

### DETAILED DESCRIPTION

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

### Definitions

For convenience, certain terms employed in the specification, examples and claims are collected here. Unless defined otherwise, all technical and scientific terms used in this disclosure have the same meanings as commonly understood by one of ordinary skill in the art to which this disclosure belongs.

The term “about” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example in a list of numerical values such as “about 49, about 50, about 55, . . . ”, “about 50” means a range extending to less than half the interval(s) between the preceding and subsequent values, e.g., more than 49.5 to less than 50.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

The terms “administer,” “administering” or “administration” as used herein refer to administering a compound or pharmaceutically acceptable salt of the compound or a composition or formulation comprising the compound or pharmaceutically acceptable salt of the compound to a patient.

The term “pharmaceutically acceptable salts” includes both acid and base addition salts. Pharmaceutically acceptable salts include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Base addition salts include but are not limited to, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e. g., lysine and arginine dicyclohexylamine and the like. Examples of metal salts include lithium, sodium, potassium, magnesium, calcium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like. Examples of organic bases include lysine, arginine, guanidine, diethanolamine, choline and the like. Those skilled in the art will further recognize that acid addition salts may be prepared by reaction of the compounds with the appropriate inorganic or organic acid via any of a number of known methods.

The term “treating” as used herein with regard to a patient, refers to improving at least one symptom of the patient's disorder. Treating can be improving, or at least partially ameliorating a disorder or an associated symptom of a disorder.

The terms “effective amount” and “therapeutically effective amount” are used interchangeably in this disclosure and refer to an amount of a compound, or a salt thereof, (or pharmaceutical composition containing the compound or salt) that, when administered to a patient, is capable of performing the intended result. The “effective amount” will vary depending on the active ingredient, the state, disorder, or condition to be treated and its severity, and the age, weight, physical condition and responsiveness of the mammal to be treated.

The term “therapeutically effective” applied to dose or amount refers to that quantity of a compound or pharmaceutical formulation that is sufficient to result in a desired clinical benefit after administration to a patient in need thereof.

The term “carrier” or “vehicle” as used interchangeably herein encompasses carriers, excipients, adjuvants, and diluents or a combination of any of the foregoing, meaning a material, composition or vehicle, such as a liquid or solid filler, diluent, excipient, solvent or encapsulating material involved in carrying or transporting a pharmaceutical agent from one organ, or portion of the body, to another organ or portion of the body. In addition to the adjuvants, excipients and diluents known to one skilled in the art, the carrier includes nanoparticles of organic and inorganic nature.

When a range of values is listed, it is intended to encompass each value and sub-range within the range. For example, “C₁-C₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” or “alkyl group” refers to a fully saturated, straight or branched hydrocarbon chain having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene” or “alkylene chain” refers to a fully saturated, straight or branched divalent hydrocarbon chain radical, and having from one to twelve carbon atoms. Non-limiting examples of C₁-C₁₂ alkylene include methylene, ethylene, propylene, n-butylene, and the like. The alkylene chain is attached to the rest of the molecule through a single bond and to a radical group (e.g., those described herein) through a single bond. The points of attachment of the alkylene chain to the rest of the molecule and to the radical group can be through one carbon or any two carbons within the chain. Unless stated otherwise specifically in the specification, an alkylene chain can be optionally substituted.

“Alkenyl” or “alkenyl group” refers to a straight or branched hydrocarbon chain having from two to twelve carbon atoms and having one or more carbon-carbon double bonds. Each alkenyl group is attached to the rest of the molecule by a single bond. Alkenyl group comprising any number of carbon atoms from 2 to 12 are included. An alkenyl group comprising up to 12 carbon atoms is a C₂-C₁₂ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene” or “alkenylene chain” refers to an unsaturated, straight or branched divalent hydrocarbon chain radical having one or more olefins and from two to twelve carbon atoms. Non-limiting examples of C₂-C₁₂ alkenylene include ethenylene, propenylene, n-butenylene, and the like. The alkenylene chain is attached to the rest of the molecule through a single bond and to a radical group (e.g., those described herein) through a single bond. The points of attachment of the alkenylene chain to the rest of the molecule and to the radical group can be through one carbon or any two carbons within the chain. Unless stated otherwise specifically in the specification, an alkenylene chain can be optionally substituted.

“Alkynyl” or “alkynyl group” refers to a straight or branched hydrocarbon chain having from two to twelve carbon atoms, and having one or more carbon-carbon triple bonds. Each alkynyl group is attached to the rest of the molecule by a single bond. Alkynyl group comprising any number of carbon atoms from 2 to 12 are included. An alkynyl group comprising up to 12 carbon atoms is a C₂-C₁₂ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise specifically in the specification, an alkyl group can be optionally substituted.

“Alkynylene” or “alkynylene chain” refers to an unsaturated, straight or branched divalent hydrocarbon chain radical having one or more alkynes and from two to twelve carbon atoms. Non-limiting examples of C₂-C₁₂ alkynylene include ethynylene, propynylene, n-butynylene, and the like. The alkynylene chain is attached to the rest of the molecule through a single bond and to a radical group (e.g., those described herein) through a single bond. The points of attachment of the alkynylene chain to the rest of the molecule and to the radical group can be through any two carbons within the chain having a suitable valency. Unless stated otherwise specifically in the specification, an alkynylene chain can be optionally substituted.

“Alkoxy” refers to a group of the formula —ORₐ where Rₐ is an alkyl, alkenyl or alknyl as defined above containing one to twelve carbon atoms. Unless stated otherwise specifically in the specification, an alkoxy group can be optionally substituted.

“Aryl” refers to a hydrocarbon ring system comprising hydrogen, 6 to 18 carbon atoms and at least one aromatic ring, and which is attached to the rest of the molecule by a single bond. For purposes of this disclosure, the aryl can be a monocyclic, bicyclic, tricyclic or tetracyclic ring system, which can include fused or bridged ring systems. Aryls include, but are not limited to, aryls derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the “aryl” can be optionally substituted.

“Aralkyl” or “arylalkyl” refers to a radical of the formula —R_{b}—R_{c} where R_{b} is an alkylene group as defined above and R_{c} is one or more aryl radicals as defined above, for example, benzyl, diphenylmethyl and the like. Unless stated otherwise specifically in the specification, an aralkyl group can be optionally substituted.

“Carbocyclyl,” “carbocyclic ring” or “carbocycle” refers to a rings structure, wherein the atoms which form the ring are each carbon, and which is attached to the rest of the molecule by a single bond. Carbocyclic rings can comprise from 3 to 20 carbon atoms in the ring. Carbocyclic rings include aryls and cycloalkyl, cycloalkenyl, and cycloalkynyl as defined herein. Unless stated otherwise specifically in the specification, a carbocyclyl group can be optionally substituted.

“Cycloalkyl” refers to a stable non-aromatic monocyclic or polycyclic fully saturated hydrocarbon consisting solely of carbon and hydrogen atoms, which can include fused, bridged, or spirocyclic ring systems, having from three to twenty carbon atoms (e.g., having from three to ten carbon atoms) and which is attached to the rest of the molecule by a single bond. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Cycloalkenyl” refers to a stable non-aromatic monocyclic or polycyclic hydrocarbon consisting solely of carbon and hydrogen atoms, having one or more carbon-carbon double bonds, which can include fused or bridged ring systems, having from three to twenty carbon atoms, preferably having from three to ten carbon atoms, and which is attached to the rest of the molecule by a single bond. Monocyclic cycloalkenyls include, for example, cyclopentenyl, cyclohexenyl, cycloheptenyl, cycloctenyl, and the like. Polycyclic cycloalkenyls include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon consisting solely of carbon and hydrogen atoms, having one or more carbon-carbon triple bonds, which can include fused or bridged ring systems, having from three to twenty carbon atoms, preferably having from three to ten carbon atoms, and which is attached to the rest of the molecule by a single bond. Monocyclic cycloalkynyl include, for example, cycloheptynyl, cyclooctynyl, and the like. Unless otherwise stated specifically in the specification, a cycloalkynyl group can be optionally substituted.

“Haloalkyl” refers to an alkyl, as defined above, that is substituted by one or more halo radicals, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

“Heterocyclyl,” “heterocyclic ring” or “heterocycle” refers to a stable saturated, unsaturated, or aromatic 3- to 20-membered ring which consists of two to nineteen carbon atoms and from one to six heteroatoms selected from the group consisting of nitrogen, oxygen and sulfur, and which is attached to the rest of the molecule by a single bond. Heterocyclyl or heterocyclic rings include heteroaryls, heterocyclylalkyls, heterocyclylalkenyls, and hetercyclylalkynyls. Unless stated otherwise specifically in the specification, the heterocyclyl can be a monocyclic, bicyclic, tricyclic or tetracyclic ring system, which can include fused, bridged, or spirocyclic ring systems; and the nitrogen, carbon or sulfur atoms in the heterocyclyl can be optionally oxidized; the nitrogen atom can be optionally quaternized; and the heterocyclyl can be partially or fully saturated. Examples of such heterocyclyl include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group can be optionally substituted.

“Heteroaryl” refers to a 5- to 20-membered ring system comprising hydrogen atoms, one to nineteen carbon atoms, one to six heteroatoms selected from the group consisting of nitrogen, oxygen and sulfur, at least one aromatic ring, including compounds with aromatic resonance structures (e.g., 2-pyridone), and which is attached to the rest of the molecule by a single bond. For purposes of this disclosure, the heteroaryl can be a monocyclic, bicyclic, tricyclic or tetracyclic ring system, which can include fused or bridged ring systems; and the nitrogen, carbon or sulfur atoms in the heteroaryl can be optionally oxidized; the nitrogen atom can be optionally quaternized. Examples include, but are not limited to, azepinyl, acridinyl, benzimidazolyl, benzothiazolyl, benzindolyl, benzofuranyl, benzooxazolyl, benzothiadiazolyl, benzo[b][1,4]dioxepinyl, 1,4 benzodioxanyl, benzonaphthofuranyl, benzoxazolyl, benzodioxolyl, benzodioxinyl, benzopyranyl, benzopyranonyl, benzofuranonyl, benzothienyl (benzothiophenyl), benzotriazolyl, benzo[4,6]imidazo[1,2 a]pyridinyl, carbazolyl, cinnolinyl, dibenzofuranyl, dibenzothiophenyl, furanyl, furanonyl, isothiazolyl, imidazolyl, indazolyl, indolyl, isoindolyl, indolinyl, isoindolinyl, isoquinolyl, indolizinyl, isoxazolyl, naphthyridinyl, oxadiazolyl, 2 oxoazepinyl, oxazolyl, oxiranyl, 1-oxidopyridinyl, 1 oxidopyrimidinyl, 1-oxidopyrazinyl, 1-oxidopyridazinyl, 1 phenyl 1H pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group can be optionally substituted.

“Heterocyclylalkyl” refers to a radical of the formula —R_{b}—Rₑ where R_{b} is an alkylene, alkenylene, or alkynylene group as defined above and Rₑ is a heterocyclyl radical as defined above. Unless stated otherwise specifically in the specification, a heterocyclylalkyl group can be optionally substituted.

The term “substituted” used herein means any of the groups described herein (e.g., alkyl, alkenyl, alkynyl, alkoxy, aryl, aralkyl, carbocyclyl, cycloalkyl, cycloalkenyl, cycloalkynyl, haloalkyl, heterocyclyl, and/or heteroaryl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted” also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halogen, alkyl, alkenyl, alkynyl, alkoxy, alkylamino, thioalkyl, aryl, aralkyl, cycloalkyl, cycloalkenyl, cycloalkynyl, cycloalkyl, haloalkyl, haloalkenyl, haloalkynyl, heterocyclyl, N-heterocyclyl, heterocyclylalkyl, heteroaryl, N-heteroaryl and/or heteroarylalkyl group. In some embodiments, “substituted” further means any alkyl, cycloalkyl or heterocyclylalkyl in which one or more hydrogen atoms is replaced by an isotope e.g., deuterium. In addition, each of the foregoing substituents can also be optionally substituted with one or more of the above substituents.

The compounds of the present disclosure may also contain unnatural proportions of atomic isotopes at one or more of the atoms that constitute such compounds. For example, the compounds may be radiolabeled with radioactive isotopes, such as for example deuterium (²H), tritium (³H), iodine-125 (¹²⁵I) or carbon-14 (¹⁴C). All isotopic variations of the compounds of the present disclosure, whether radioactive or not, are intended to be encompassed within the scope of the disclosure.

As used herein, the symbol

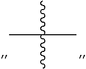

(hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

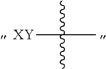

indicates that the chemical entity “XY” is bonded to another chemical entity via the point of attachment bond. Furthermore, the specific point of attachment to the non-depicted chemical entity can be specified by inference. For example, the compound CH₃—R³, wherein R³ is H or

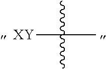

infers that when R³ is “XY”, the point of attachment bond is the same bond as the bond by which R³ is depicted as being bonded to CH₃.

### Compounds

The present disclosure provides compounds that are inverse agonists of the G protein-coupled receptor 6 (GPR6) as well as pharmaceutical compositions thereof and uses thereof in treating various diseases and disorders.

In some embodiments, the present disclosure provides a compound of Formula (I), or a pharmaceutically acceptable salt thereof:

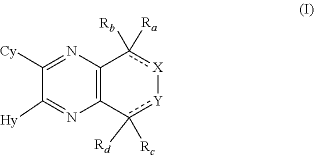

wherein
Rₐ, R_{b}, R_{c}, and R_{d} are each independently absent, H, alkyl, alkenyl, halogen, —CN, —CH₂NRₑR_{f}, —NRₑR_{f}, —NRₑC(═O)R_{f}, —NRₑC(═O)NR_{f}R_{f}, —NRₑC(═O)OR_{f}, —NRₑSO₂R_{f}, —OC(═O)NRₑR_{f}, —ORₑ, —SRₑ, —SORₑ, —SO₂Rₑ, —SO₂ORₑ, —SO₂NRₑR_{f}, —C(═O)Rₑ, —C(═O)ORₑ, —C(═O)C(CH₃)₂OH, —C(═O)NRₑR_{f}, spirocyclyl, spiroheterocyclyl, carbocyclyl, or heterocyclyl; Rₐ and R_{b} together with the atoms to which they are attached form ═NSO₂Rₑ, ═O, carbocyclyl, or heterocyclyl; R_{c} and R_{d} together with the atoms to which they are attached form ═NSO₂Rₑ, ═O, carbocyclyl, or heterocyclyl; or Rₐ and R_{c} together with the atoms to which they are attached form a carbocyclyl or heterocyclyl; Rₐ and Y together with the atoms to which they are attached form a carbocyclyl or heterocyclyl; R_{c} and X together with the atoms to which they are attached form a carbocyclyl or heterocyclyl;
each of X and Y is independently O, N, NRₑ, N(C(Rₑ)₂C(═O)NHR_{f}), N(C(═O)Rₑ), N(C(═O)ORₑ), NC(═O)C(Rₑ)₃, NC(═O)C(Rₑ)₂(Rₑ)₃, S, S(═O), S(═O)₂, S(═O)(═NRₑ), S(═NRₑ)₂, C(CN), C(CN)Rₑ, C(C(═O)Rₑ), C(Rₕ), NS(O)₂NRₑRₕ, NS(O)NRₑRₕ, C(═NSO₂Rₑ), or C(Rₕ)₂; provided that X and Y cannot both be selected from C(CN), C(CN)Rₑ, C(C(═O)Rₑ) and C(Rₕ);
each Rₑ, R_{f}, and R_{g} is independently H, halogen, —OH, —CN, —OCHF₂, —C(CH₃)₂OH, -haloalkyl, —CH₂OH, CH(CH₃)CN, —C((CH₃)₂CN), C(═O)CH₃, —NH₂, NH(alkyl), —CH₂CH₂OCH₃, —CH₂CH(OH)CH₂OH, alkoxy, alkyl, carbocyclyl, or heterocyclyl,
each Rₕ is independently H, halogen, OH, —CN, alkyl, —C(Rₑ)₃, —C(Rₑ)₂C(Rₑ)₃, —CH₂C(Rₑ)₂C(Rₑ)₃, C(═O)C(Rₑ)₃, —OCH₂C(Rₑ)₃, —NHCH₂C(Rₑ)₃, —N(alkyl)CH₂C(Rₑ)₃, —O-heterocyclyl, alkoxy, haloalkoxy, —NRₑR_{f}, —NRₑC(═O)R_{f}, —NRₑC(═O)NR_{f}R_{g}, —CH₂NRₑC(═O)NR_{f}R_{g}, —CH₂C(═O)NRₑR_{f}, —NRₑC(═O)ORₑ, —NRₑSO₂R_{f}, —OC(═O)NRₑR_{f}, —ORₑ, —SRₑ, —SORₑ, —SO₂Rₑ, —SO₂ORₑ, —SO₂NRₑR_{f}, —C(═O)Rₑ, —C(═O)ORₑ, —C(═O)NRₑR_{f}, carbocyclyl, aryl or heterocyclyl; or two Rₕ attached to the same carbon atom together with the atoms to which they are attached form ═NSO₂Rₑ, ═O, carbocyclyl, heteroaryl or heterocyclyl;
Hy is a carbon-linked 3-, 4-, 5- or 6-membered carbocyclyl; a carbon-linked 3-10-membered monocyclic heterocyclyl; or a carbon-linked 3-10-membered bicyclic heterocyclyl; and
Cy is -A-L-B or -A=L-B;
wherein
A is a nitrogen-containing heterocyclylene linked by a nitrogen atom to

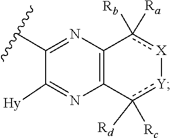

B is a 5- to 7-membered heterocyclyl or a 5- to 7-membered carbocyclyl; and
L is absent, C(Rₕ), C(Rₕ)₂, OC(Rₕ)₂, C(═O), C(═NSO₂Rₑ), C(═O)N(Rₑ), NRₑ, O, S, N, S(═O), S(═O)₂, S(═O)(═NRₑ), or S(═NRₑ)₂, wherein when L is absent or NRₑ, B is aryl or heteroaryl.

In some embodiments, the present disclosure provides a compound of Formula (I), or a pharmaceutically acceptable salt thereof:

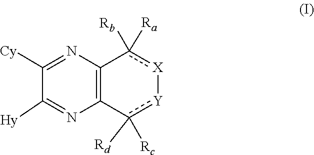

wherein
Rₐ, R_{b}, R_{c}, and R_{d} are each independently absent, H, alkyl, alkenyl, halogen, —CN, —CH₂NRₑR_{f}, —NRₑR_{f}, —NRₑC(═O)R_{f}, —NRₑC(═O)NR_{f}R_{g}, —NRₑC(═O)OR_{f}, —NRₑSO₂R_{f}, —OC(═O)NRₑR_{f}, —ORₑ, —SRₑ, —SORₑ, —SO₂Rₑ, —SO₂ORₑ, —SO₂NRₑR_{f}, —C(═O)Rₑ, —C(═O)ORₑ, —C(═O)C(CH₃)₂OH, —C(═O)NRₑR_{f}, spirocyclyl, spiroheterocyclyl, carbocyclyl, or heterocyclyl; Rₐ and R_{b} together with the atoms to which they are attached form ═NSO₂Rₑ, ═O, carbocyclyl, or heterocyclyl; R_{c} and R_{d} together with the atoms to which they are attached form ═NSO₂Rₑ, ═O, carbocyclyl, or heterocyclyl; or Rₐ and R_{c} together with the atoms to which they are attached form a carbocyclyl or heterocyclyl; Rₐ and Y together with the atoms to which they are attached form a carbocyclyl or heterocyclyl; R_{c} and X together with the atoms to which they are attached form a carbocyclyl or heterocyclyl;
each of X and Y is independently O, N, NRₑ, N(C(Rₑ)₂C(═O)NHR_{f}), N(C(═O)Rₑ), N(C(═O)ORₑ), NC(═O)C(Rₑ)₃, NC(═O)C(Rₑ)₂(Rₑ)₃, S, S(═O), S(═O)₂, S(═O)(═NRₑ), S(═NRₑ)₂, C(CN), C(CN)Rₑ, C(C(═O)Rₑ), C(Rₕ), NS(O)₂NRₑRₕ, NS(O)NRₑRₕ, C(═NSO₂Rₑ), or C(Rₕ)₂;
each Rₑ, R_{f}, and R_{g} is independently H, halogen, —OH, —CN, —OCHF₂, —C(CH₃)₂OH, —CHF₂, —CF₃, —CH₂OH, CH(CH₃)CN, —C((CH₃)₂CN), —NH₂, —CH₂CH₂OCH₃, —CH₂CH(OH)CH₂OH, alkoxy, alkyl, carbocyclyl, or heterocyclyl,
each Rₕ is independently H, halogen, OH, —CN, alkyl, —C(Rₑ)₃, —C(Rₑ)₂C(Rₑ)₃, —CH₂C(Rₑ)₂C(Rₑ)₃, —OCH₂C(Rₑ)₃, —NHCH₂C(Rₑ)₃, —N(alkyl)CH₂C(Rₑ)₃, —O-heterocyclyl, alkoxy, haloalkoxy, —NRₑR_{f}, —NRₑC(═O)R_{f}, —NRₑC(═O)NR_{f}R_{g}, —CH₂NRₑC(═O)NR_{f}R_{g}, —NRₑC(═O)OR_{f}, —NRₑSO₂R_{f}, —OC(═O)NRₑR_{f}, —ORₑ, —SRₑ, —SORₑ, —SO₂Rₑ, —SO₂ORₑ, —SO₂NRₑR_{f}, —C(═O)Rₑ, —C(═O)ORₑ, —C(═O)NRₑR_{f}, carbocyclyl, aryl or heterocyclyl; or
two Rₕ attached to the same carbon atom together with the atoms to which they are attached form ═NSO₂Rₑ, ═O, carbocyclyl, heteroaryl or heterocyclyl;
Hy is a carbon-linked 3-, 4-, 5- or 6-membered carbocyclyl; a carbon-linked 3-10-membered monocyclic heterocyclyl; or a carbon-linked 3-10-membered bicyclic heterocyclyl; and
Cy is -A-L-B or -A=L-B;
wherein
A is
a nitrogen-containing heterocyclylene linked by a nitrogen atom to

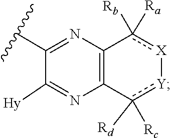

or a 6-membered cycloalkylene or cycloalkenylene;
B is a 5- to 7-membered heterocyclyl, a 5- to 7-membered carbocyclyl, —C(═O)C₁₋₆alkyl or H, and
L is absent, C(Rₕ), C(Rₕ)₂, OC(Rₕ)₂, C(═O), C(═NSO₂Rₑ), C(═O)N(Rₑ), NRₑ, O, S, N, S(═O), S(═O)₂, S(═O)(═NRₑ), or S(═NRₑ)₂, wherein when L is absent or NRₑ, B is aryl or heteroaryl.

In embodiments, the present disclosure provides a compound of Formula (I), or a pharmaceutically acceptable salt thereof:

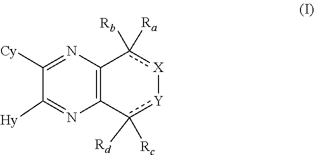

wherein
Rₐ, R_{b}, R_{c}, and R_{d} are each independently absent, H, alkyl, halogen, —CN, —CH₂NRₑR_{f}, —NRₑR_{f}, —NRₑC(═O)R_{f}, —NRₑC(═O)NR_{f}R_{g}, —NRₑC(═O)OR_{f}, —NRₑSO₂R_{f}, —OC(═O)NRₑR_{f}, —ORₑ, —SRₑ, —SORₑ, —SO₂Rₑ, —SO₂ORₑ, —SO₂NRₑR_{f}, —C(═O)Rₑ, —C(═O)ORₑ, —C(═O)NRₑR_{f}, (hetero)alkyl, (hetero)alkenyl, (hetero)alkynyl, spirocyclyl, spiroheterocyclyl, carbocyclyl, or heterocyclyl; Rₐ and R_{b} together with the atoms to which they are attached form ═NSO₂Rₑ, ═O, carbocyclyl, or heterocyclyl; R_{c} and R_{d} together with the atoms to which they are attached form ═NSO₂Rₑ, ═O, carbocyclyl, or heterocyclyl; Rₐ and R_{c} together with the atoms to which they are attached form a carbocyclyl or heterocyclyl; Rₐ and Y together with the atoms to which they are attached form a carbocyclyl or heterocyclyl; R_{c} and X together with the atoms to which they are attached form a carbocyclyl or heterocyclyl;
each of X and Y is independently selected from the group consisting of O, N, NRₑ, N(C(Rₑ)₂C(═O)NHR_{f}), N(C(═O)Rₑ), N(C(═O)ORₑ), NC(═O)C(Rₑ)₃, S, S(═O), S(═O)₂, S(═O)(═NRₑ), S(═NRₑ)₂, NS(═O)₂Rₑ, C(CN), C(CN)Rₑ, C(C(═O)Rₑ), C(Rₕ), NS(O)₂NRₑRₕ, NS(O)NRₑRₕ, C(═NSO₂Rₑ), and C(Rₕ)₂;
each Rₑ, R_{f}, and R_{g} is independently H, —CN, —OCHF₂, alkoxy, alkyl, (hetero)alkyl, (hetero)alkenyl, (hetero)alkynyl, carbocyclyl, or heterocyclyl
Hy is a carbon-linked 3-, 4-, 5- or 6-membered carbocyclic ring or a carbon-linked 3-, 4-, 5- or 6-membered heterocyclic ring comprising at least one N; and
Cy is -A-L-B;
wherein
A is a saturated 4- to 7-membered hetero- or carbocyclic ring, a saturated spirocyclic or spiroheterocyclic ring;
B is a 5- to 7-membered hetero- or carbocyclic ring; and
L is absent, C(Rₕ)₂, C(═O), C(═NSO₂Rₑ), NRₑ, O, S, N, S(═O), S(═O)₂, S(═O)(═NRₑ), or S(═NRₑ)₂, wherein when L is absent or NRₑ, B is aryl or heteroaryl,
wherein
each Rₕ is independently H, halogen, —CN, —OCHF₂, alkyl, alkoxy, —NRₑR_{f}, —NRₑC(═O)R_{f}, —NRₑC(═O)NR_{f}R_{g}, —NRₑC(═O)OR_{f}, —NRₑSO₂R_{f}, —OC(═O)NRₑR_{f}, —ORₑ, —SRₑ, —SORₑ, —SO₂Rₑ, —SO₂ORₑ, —SO₂NRₑR_{f}, —C(═O)Rₑ, —C(═O)ORₑ, —C(═O)NRₑR_{f}, (hetero)alkyl, (hetero)alkenyl, (hetero)alkynyl, carbocyclyl, or heterocyclyl; or two Rₕ attached to the same carbon atom together with the atoms to which they are attached form ═NSO₂Rₑ, ═O, carbocyclyl, or heterocyclyl.

In embodiments, provided herein is a compound of Formula (I′), or a pharmaceutically acceptable salt thereof:

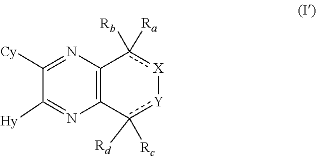

wherein
Rₐ, R_{b}, R_{c}, and R_{d} are each independently absent, H, alkyl, halogen, —CN, —CH₂NRₑR_{f}, —NRₑR_{f}, —NRₑC(═O)R_{f}, —NRₑC(═O)NR_{f}R_{g}, —NRₑC(═O)OR_{f}, —NRₑSO₂R_{f}, —OC(═O)NRₑR_{f}, —ORₑ, —SRₑ, —SORₑ, —SO₂Rₑ, —SO₂ORₑ, —SO₂NRₑR_{f}, —C(═O)Rₑ, —C(═O)ORₑ, —C(═O)NRₑR_{f}, (hetero)alkyl, (hetero)alkenyl, (hetero)alkynyl, spirocyclyl, spiroheterocyclyl, carbocyclyl, or heterocyclyl; Rₐ and R_{b} together with the atoms to which they are attached form ═NSO₂Rₑ, ═O, carbocyclyl, or heterocyclyl; R_{c} and R_{d} together with the atoms to which they are attached form ═NSO₂Rₑ, ═O, carbocyclyl, or heterocyclyl; or Rₐ and R_{c} together with the atoms to which they are attached form a carbocyclyl or heterocyclyl; Rₐ and Y together with the atoms to which they are attached form a carbocyclyl or heterocyclyl; R_{c} and X together with the atoms to which they are attached form a carbocyclyl or heterocyclyl;
each of X and Y is independently selected from the group consisting of O, N, NRₑ, N(C(Rₑ)₂C(═O)NHR_{f}), N(C(═O)Rₑ), N(C(═O)ORₑ), NC(═O)C(Rₑ)₃, S, S(═O), S(═O)₂, S(═O)(═NRₑ), S(═NRₑ)₂, NS(═O)₂Rₑ, C(CN), C(CN)Rₑ, C(C(═O)Rₑ), C(Rₕ), NS(O)₂NRₑRₕ, NS(O)NRₑRₕ, C(═NSO₂Rₑ), and C(Rₕ)₂, wherein at least one of X and Y is O, N, NRₑ, N(C(Rₑ)₂C(═O)NHR_{f}), N(C(═O)Rₑ), N(C(═O)ORₑ), NC(═O)C(Rₑ)₃, S, S(═O), S(═O)₂, S(═O)(═NRₑ), S(═NRₑ)₂, NS(═O)₂Rₑ, NS(O)₂NRₑRₕ, or NS(O)NRₑRₕ;
each Rₑ, R_{f}, and R_{g} is independently H, —OH, —CN, —OCHF₂, —CF₃, —CH₂OH, CH(CH₃)CN, —C((CH₃)₂CN), —NHCH₃, —N(CH₃)₂, —CH₂CH(OH)CH₂OH, ═O, alkoxy, alkyl, (hetero)alkyl, (hetero)alkenyl, (hetero)alkynyl, carbocyclyl, or heterocyclyl;
Hy is a carbon-linked 3-, 4-, 5- or 6-membered carbocyclic ring or a carbon-linked 3-, 4-, 5- or 6-membered heterocyclic ring comprising one or more S or O; and
Cy is -A-L-B;
wherein
A is a piperidine or piperazine ring;
B is a 5- to 7-membered hetero- or carbocyclic ring; and
L is absent, C(Rₕ)₂, C(═O), C(═NSO₂Rₑ), NRₑ, O, S, N, S(═O), S(═O)₂, S(═O)(═NRₑ), or S(═NRₑ)₂, wherein when L is absent or NRₑ, B is aryl or heteroaryl,
wherein
each Rₕ is independently H, halogen, —CN, —OCHF₂, alkyl, alkoxy, —CH₂OH, —CH(OH)C(Rₑ)₃, —CH(OH)CRₑ, —NRₑR_{f}, —NRₑC(═O)R_{f}, —NRₑC(═O)NR_{f}R_{g}, —NRₑC(═O)OR_{f}, —NRₑSO₂R_{f}, —OC(═O)NRₑR_{f}, —ORₑ, —SRₑ, —SORₑ, —SO₂Rₑ, —SO₂ORₑ, —SO₂NRₑR_{f}, —C(═O)Rₑ, —C(═O)ORₑ, —C(═O)NRₑR_{f}, (hetero)alkyl, (hetero)alkenyl, (hetero)alkynyl, carbocyclyl, or heterocyclyl; or two Rₕ attached to the same carbon atom together with the atoms to which they are attached form ═NSO₂Rₑ, ═O, carbocyclyl, or heterocyclyl.

In embodiments, provided herein is a compound of Formula (Ia), or a pharmaceutically acceptable salt thereof:

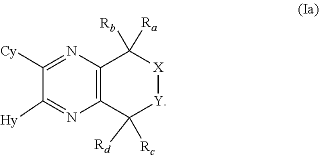

In embodiments, provided herein is a compound of Formula (Ib), or a pharmaceutically acceptable salt thereof:

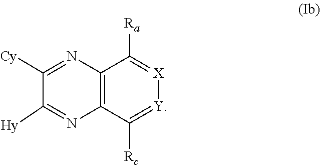

In embodiments, provided herein is a compound of Formula (Ic), or a pharmaceutically acceptable salt thereof:

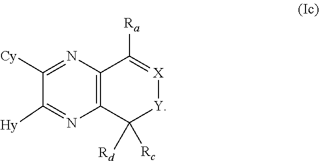

In embodiments, provided herein is a compound of Formula (Id), or a pharmaceutically acceptable salt thereof:

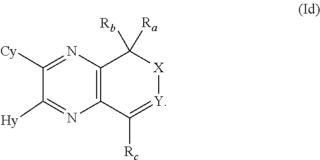

In embodiments, Rₐ, R_{b}, R_{c}, and R_{d} are H.

In embodiments, Rₐ and R_{c} are H.

In embodiments Rₐ, R_{c}, and R_{d} are H.

In embodiments Rₐ, R_{b}, and R_{c} are H.

In some embodiments, the present disclosure provides a compound of Formula (Ib), or a pharmaceutically acceptable salt thereof:

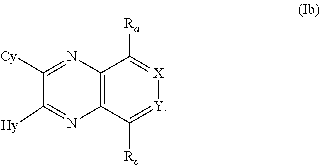

wherein
Rₐ and R_{c} are each independently H, alkyl, alkenyl, halogen, —CN, —CH₂NRₑR_{f}, —NRₑR_{f}, —NRₑC(═O)R_{f}, —NRₑC(═O)NR_{f}R_{g}, —NRₑC(═O)OR_{f}, —NRₑS(═O)₂R_{f}, —OC(═O)NRₑR_{f}, —ORₑ, —SRₑ, —S(═O)Rₑ, —S(═O)₂Rₑ, —S(═O)₂ORₑ, —S(═O)₂NRₑR_{f}, —C(═O)Rₑ, —C(═O)ORₑ, —C(═O)C(CH₃)₂OH, —C(═O)NRₑR_{f}, spirocyclyl, spiroheterocyclyl, carbocyclyl, or heterocyclyl;
each of X and Y is independently N, C(CN), C(C(═O)Rₑ), or C(Rₕ);
each Rₑ, R_{f}, and R_{g} is independently H, halogen, —OH, —CN, —OCHF₂, —C(CH₃)₂OH, -haloalkyl, —CH₂OH, CH(CH₃)CN, —C((CH₃)₂CN), C(═O)CH₃, —NH₂, NH(alkyl), —CH₂CH₂OCH₃, —CH₂CH(OH)CH₂OH, alkoxy, alkyl, carbocyclyl, or heterocyclyl,
each Rₕ is independently H, halogen, OH, —CN, alkyl, —C(Rₑ)₃, —C(Rₑ)₂C(Rₑ)₃, —CH₂C(Rₑ)₂C(Rₑ)₃, C(═O)C(Rₑ)₃, —OCH₂C(Rₑ)₃, —NHCH₂C(Rₑ)₃, —N(alkyl)CH₂C(Rₑ)₃, —O-heterocyclyl, alkoxy, haloalkoxy, —NRₑR_{f}, —NRₑC(═O)R_{f}, —NRₑC(═O)NR_{f}R_{g}, —CH₂NRₑC(═O)NR_{f}R_{g}, —CH₂C(═O)NRₑR_{f}, —NRₑC(═O)OR_{f}, —NRₑSO₂R_{f}, —OC(═O)NRₑR_{f}, —ORₑ, —SRₑ, —SORₑ, —SO₂Rₑ, —SO₂ORₑ, —SO₂NRₑR_{f}, —C(═O)Rₑ, —C(═O)ORₑ, —C(═O)NRₑR_{f}, carbocyclyl, aryl or heterocyclyl; or two Rₕ attached to the same carbon atom together with the atoms to which they are attached form ═NSO₂Rₑ, ═O, carbocyclyl, heteroaryl or heterocyclyl;
Hy is a carbon-linked 3-, 4-, 5- or 6-membered carbocyclyl; a carbon-linked 3-10-membered monocyclic heterocyclyl; or a carbon-linked 3-10-membered bicyclic heterocyclyl; and
Cy is -A-L-B or -A=L-B;
wherein
A is a nitrogen-containing heterocyclylene linked by a nitrogen atom to

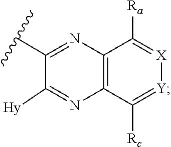

and
B is a 5- to 7-membered heterocyclyl or a 5- to 7-membered carbocyclyl; and
L is absent, C(Rₕ), C(Rₕ)₂, OC(Rₕ)₂, C(═O), C(═O)N(Rₑ), C(═NSO₂Rₑ), NRₑ, O, S, N, S(═O), S(═O)₂, S(═O)(═NRₑ), or S(═NRₑ)₂, wherein when L is absent or NRₑ, B is aryl or heteroaryl.

In some embodiments, the present disclosure provides a compound of formula (Ia), or a pharmaceutically acceptable salt thereof:

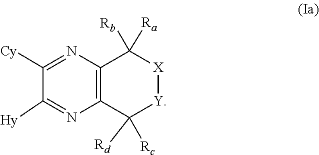

wherein
R_{b} and R_{d}, are each independently H;
Rₐ and R_{c}, are each independently H, alkyl, halogen, —CN, —CH₂NRₑR_{f}, —NRₑR_{f}, —NRₑC(═O)R_{f}, —NRₑC(═O)NR_{f}R_{g}, —NRₑC(═O)OR_{f}, —NRₑSO₂R_{f}, —OC(═O)NRₑR_{f}, —ORₑ, —SRₑ, —SORₑ, —SO₂Rₑ, —SO₂ORₑ, —SO₂NRₑR_{f}, —C(═O)Rₑ, —C(═O)ORₑ, —C(═O)NRₑR_{f}, spirocyclyl, spiroheterocyclyl, carbocyclyl, or heterocyclyl; or Rₐ and R_{c} together with the atoms to which they are attached form a carbocyclyl or heterocyclyl; Rₐ and Y together with the atoms to which they are attached form a carbocyclyl or heterocyclyl; R_{c} and X together with the atoms to which they are attached form a carbocyclyl or heterocyclyl;
each of X and Y is independently selected from the group consisting of O, NRₑ, N(C(Rₑ)₂C(═O)NHR_{f}), N(C(═O)Rₑ), N(C(═O)ORₑ), NC(═O)C(Rₑ)₃, NC(═O)C(Rₑ)₂(Rₑ)₃, S, S(═O), S(═O)₂, S(═O)(═NRₑ), S(═NRₑ)₂, NS(═O)₂Rₑ, NS(═O)₂NRₑRₕ, NS(O)NRₑRₕ, C(CN)Rₑ, C(═NSO₂Rₑ), and C(Rₕ)₂; provided that X and Y cannot both be C(CN)Rₑ, C(═NSO₂Rₑ), or C(Rₕ)₂;
each Rₑ, R_{f}, and R_{g} is independently H, —OH, —CN, —OCHF₂, —CHF₂, —CF₃, —CH₂OH, CH(CH₃)CN, —C((CH₃)₂CN), —NHCH₃, —N(CH₃)₂, —CH₂CH₂OCH₃, —CH₂CH(OH)CH₂OH, alkoxy, alkyl, carbocyclyl, or heterocyclyl;
each Rₕ is independently H, halogen, —CN, —OCHF₂, alkyl, alkoxy, —C(═O)C(Rₑ)₃, NRₑR_{f}, —NRₑC(═O)R_{f}, —NRₑC(═O)NR_{f}R_{g}, —NRₑC(═O)OR_{f}, —NRₑSO₂R_{f}, —OC(═O)NRₑR_{f}, —ORₑ, —SRₑ, —SORₑ, —SO₂Rₑ, —SO₂ORₑ, —SO₂NRₑR_{f}, —C(═O)Rₑ, —C(═O)ORₑ, —C(═O)NRₑR_{f}, carbocyclyl, or heterocyclyl; or two Rₕ attached to the same carbon atom together with the atoms to which they are attached form ═NSO₂Rₑ, ═O, carbocyclyl, or heterocyclyl.
Hy is a carbon-linked 3-, 4-, 5- or 6-membered carbocyclic ring or a carbon-linked 3-10-membered heterocyclic ring comprising one or more N, S or O; and
Cy is -A-L-B or -A=L-B;
wherein
A is a nitrogen-containing heterocyclylene linked by a nitrogen atom to

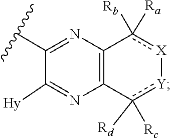

and
B is a 5- to 7-membered hetero- or carbocyclic ring; and
L is absent, C(Rₕ)₂, C(═O), C(═NSO₂Rₑ), NRₑ, O, S, N, S(═O), S(═O)₂, S(═O)(═NRₑ), or S(═NRₑ)₂, wherein when L is absent or NRₑ, B is aryl or heteroaryl.

In some embodiments, the presents disclosure provides compound of Formula (Ia), or a pharmaceutically acceptable salt thereof:

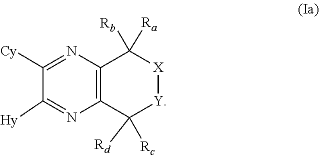

wherein
Rₐ, R_{b}, R_{c}, and R_{d}, are H;
each of X and Y is independently selected from the group consisting of O, NRₑ, N(C(Rₑ)₂C(═O)NHR_{f}), N(C(═O)Rₑ), N(C(═O)ORₑ), NC(═O)C(Rₑ)₃, NC(═O)C(Rₑ)₂(Rₑ)₃, S, S(═O), S(═O)₂, S(═O)(═NRₑ), S(═NRₑ)₂, NS(═O)₂Rₑ, NS(═O)₂NRₑRₕ, NS(═O)NRₑRₕ, C(CN)Rₑ, C(═NSO₂Rₑ), and C(Rₕ)₂; provided that X and Y cannot both be C(CN)Rₑ, C(═NSO₂Rₑ) or C(Rₕ)₂,
each Rₑ, R_{f}, and R_{g} is independently H, —OH, —CN, —OCHF₂, —CHF₂, —CF₃, —CH₂OH, CH(CH₃)CN, —C((CH₃)₂CN), —NHCH₃, —N(CH₃)₂, —CH₂CH(OH)CH₂OH, alkoxy, alkyl, carbocyclyl, or heterocyclyl;
Hy is a carbon-linked 3-, 4-, 5- or 6-membered carbocyclic ring or a carbon-linked 3-10-membered heterocyclic ring comprising one or more N, S or O; and
Cy is -A-L-B;
wherein
A is a nitrogen-containing heterocyclylene linked by a nitrogen atom to

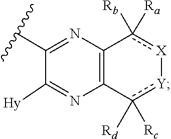

B is a 5- to 7-membered hetero- or carbocyclic ring; and
L is C(Rₕ)₂, or O, and
each Rₕ is independently H, halogen, —CN, —OCHF₂, alkyl, alkoxy, —CH₂OH, —CH(OH)C(Rₑ)₃, —CH(OH)Rₑ, —NRₑR_{f}, —NRₑC(═O)R_{f}, —NRₑC(═O)NR_{f}R_{g}, —NRₑC(═O)OR_{f}, —NRₑSO₂R_{f}, —OC(═O)NRₑR_{f}, —ORₑ, —SRₑ, —SORₑ, —SO₂Rₑ, —SO₂ORₑ, —SO₂NRₑR_{f}, —C(═O)Rₑ, —C(═O)ORₑ, —C(═O)NRₑR_{f}, carbocyclyl, or heterocyclyl; or two Rₕ attached to the same carbon atom together with the atoms to which they are attached form ═NSO₂Rₑ, ═O, carbocyclyl, or heterocyclyl.

In some embodiments, one of X and Y is C(CN), C(Rₑ), or C(C(═O)Rₑ) or C(Rₕ) and the other one of X and Y is CH or N. In some embodiments, X is C(Rₕ). In some embodiments, Y is C(Rₕ).

In some embodiments, one of X and Y is C(CN), C(Rₑ), or C(C(═O)Rₑ) or C(Rₕ) and the other one of X and Y is N. In some of these embodiments, one of X and Y is C(Rₕ), and the other of X and Y is N. In some embodiments, X is C(Rₕ), and Y is N. In some embodiments, Y is C(Rₕ), and X is N.

In some embodiments, one of X and Y is C(CN), C(Rₑ), or C(C(═O)Rₑ) or C(Rₕ). In some embodiments, the other one of X and Y is CH or N. In some embodiments, the other one of X and Y is N.

In some embodiments, one of X and Y is C(Rₕ), and the other of X and Y is N. In some embodiments, X is C(Rₕ), and Y is N. In some embodiments, Y is C(Rₕ), and X is N.

In some embodiments, one of X and Y is N, and the other one of X and Y is C(Rₕ), and Rₕ is H, alkyl, heterocyclyl, —C(Rₑ)₃, —C(Rₑ)₂C(Rₑ)₃, —CH₂C(Rₑ)₂C(Rₑ)₃, —OCH₂C(Rₑ)₃ or C(═O)C(Rₑ)₃.

In some embodiments, one of X and Y is N, and the other one of X and Y is C(Rₕ), and Rₕ is —C(Rₑ)₃, —C(Rₑ)₂C(Rₑ)₃, —CH₂C(Rₑ)₂C(Rₑ)₃, —OCH₂C(Rₑ)₃ or C(═O)C(Rₑ)₃.

In some embodiments, one of X and Y is N, and the other one of X and Y is C(Rₕ), and Rₕ is

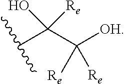

In some embodiments, one of X and Y is N, and the other one of X and Y is C(Rₕ), and Rₕ is

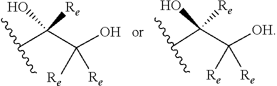

In certain of these embodiments, Rₑ is H, alkyl, OH, alkoxy, halogen, CN, or cycloalkyl. For example, Rₑ can be H, alkyl or OH.

In some embodiments, Rₕ is H, alkyl, heterocyclyl, —C(Rₑ)₃, —C(Rₑ)₂C(Rₑ)₃, —CH₂C(Rₑ)₂C(Rₑ)₃, —OCH₂C(Rₑ)₃, —NHCH₂C(Rₑ)₃, —N(alkyl)CH₂C(Rₑ)₃ or C(═O)C(Rₑ)₃. In certain embodiments, Rₕ is —C(Rₑ)₃, —C(Rₑ)₂C(Rₑ)₃, —CH₂C(Rₑ)₂C(Rₑ)₃, —OCH₂C(Rₑ)₃ or C(═O)C(Rₑ)₃.

In some embodiments, Rₕ is

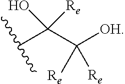

In some embodiments, Rₕ is

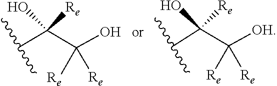

In some of the embodiments of Rₕ, Rₑ is H, alkyl, OH, alkoxy, halogen, CN, or cycloalkyl. In some of the embodiments of Rₕ, Rₑ is H, alkyl or OH. In some of the embodiments of Rₕ, Rₑ is H or alkyl.

In some embodiments, Rₕ is

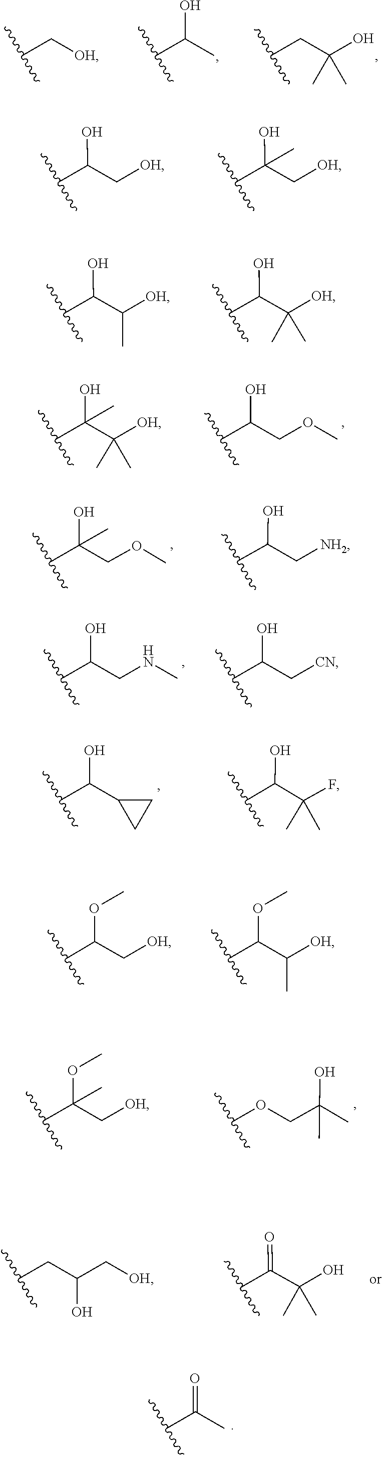

In some embodiments, Rₕ is

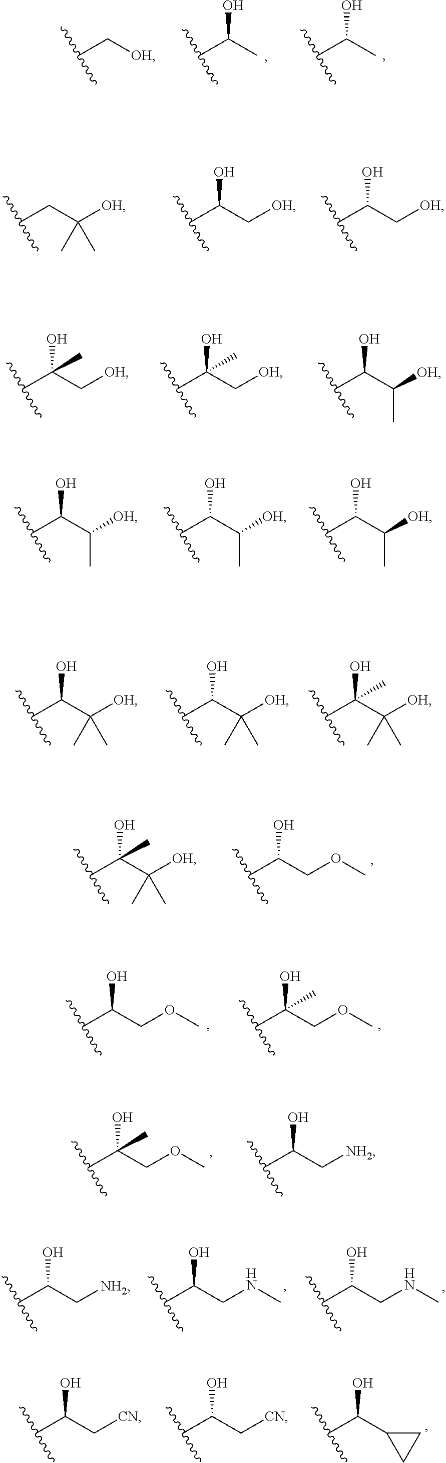

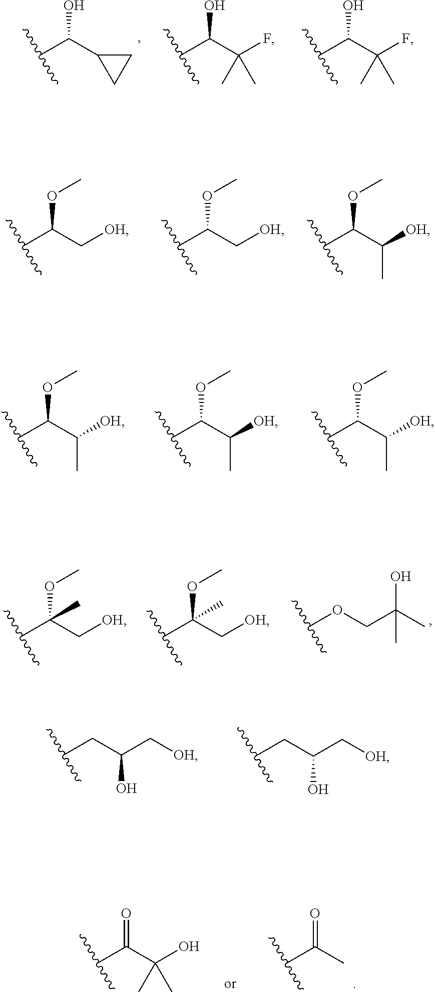

In some embodiments, Rₕ is

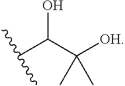 [SMILES: *C(O)C(C)(C)O]

For example, Rₕ can be

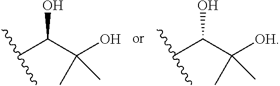

In some embodiments, one of X and Y is N, and the other one of X and Y is C(Rₕ), and Rₕ is

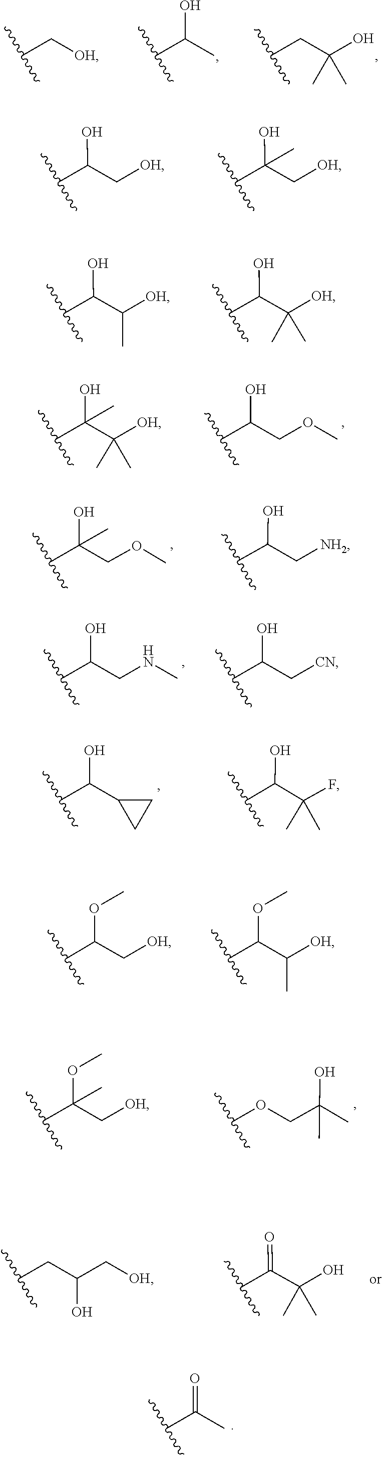

In some embodiments, one of X and Y is N, and the other one of X and Y is C(Rₕ), and Rₕ is

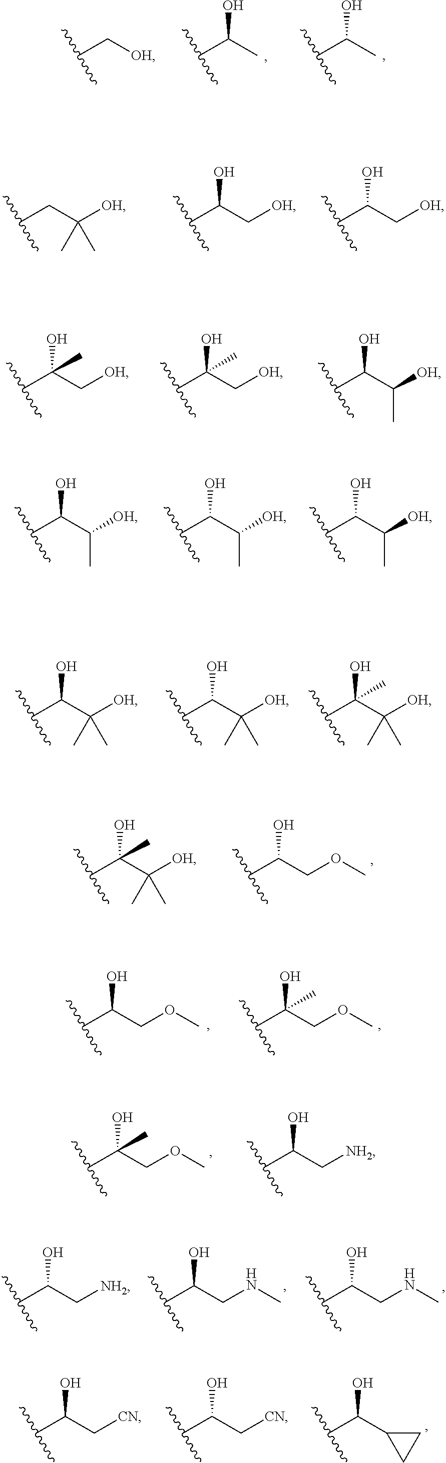

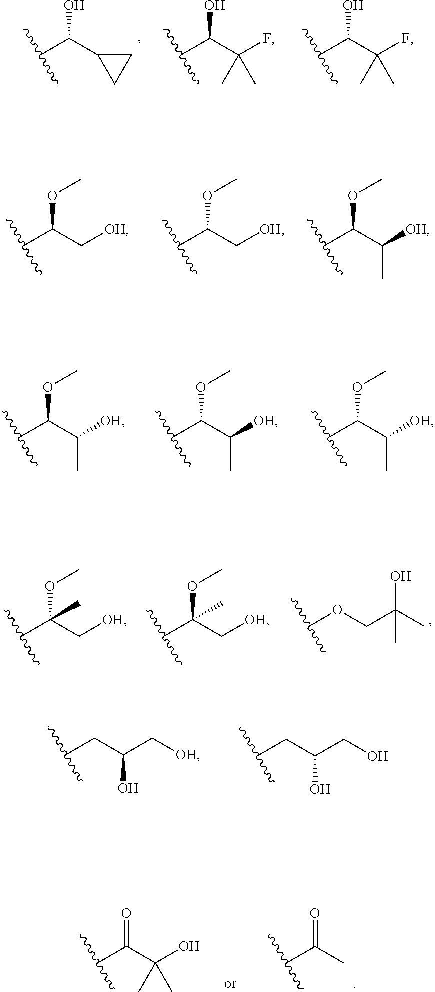

In some embodiments, one of X and Y is N, and the other one of X and Y is C(Rₕ), and Rₕ is

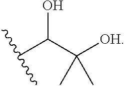 [SMILES: *C(O)C(C)(C)O]

In some embodiments, one of X and Y is N, and the other one of X and Y is C(Rₕ), and Rₕ is

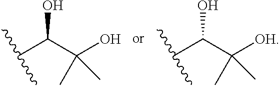

In some embodiments, one of X is N, and Y is C(Rₕ), and Rₕ is

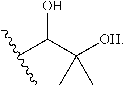 [SMILES: *C(O)C(C)(C)O]

In some embodiments, one of X is N, and Y is C(Rₕ), and Rₕ is

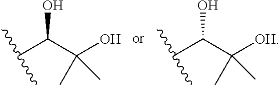

In some embodiments, one of Y is N, and X is C(Rₕ), and Rₕ is

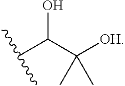 [SMILES: *C(O)C(C)(C)O]

In some embodiments, one of Y is N, and X is C(Rₕ), and Rₕ is

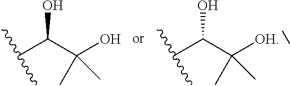

In some embodiments, one of X and Y is N(C(═O)Rₑ), and Rₑ is (hetero)alkyl, (hetero)alkenyl, (hetero)alkynyl, carbocyclyl, or heterocyclyl; and the other one of X and Y is CH₂.

In some embodiments, one of X and Y is C(CN), C(Rₑ), or C(C(═O)Rₑ). In some embodiments, X is CH or N, and Y is C(CN), C(Rₑ), or C(C(═O)Rₑ). In some embodiments, Y is CH or N, and X is C(CN), C(Rₑ), or C(C(═O)Rₑ).

In embodiments, X is N(C(═O)Rₑ). In embodiments, wherein Y is N(C(═O)Rₑ).

In some embodiments, one of X and Y is NRₑ, N(C(Rₑ)₂C(═O)NHR_{f}), N(C(═O)Rₑ), N(C(═O)ORₑ), NC(═O)C(Rₑ)₃, NC(═O)C(Rₑ)₂(Rₑ)₃, NS(═O)₂Rₑ, NS(═O)₂NRₑRₕ, or NS(O)NRₑRₕ. In some embodiments, one of X and Y is —N(alkyl), —N(heterocycle)-N—CN, —N—CH₂OH, —NC(═O)CH₃, —NCH₂CN, —NS(═O)₂CH₃, —NC(═O)OCH₃, or —NC(═O)OCH₂CH₃, —NC(═O)CH₂OH, —NC(═O)C(CH₃)OH, —NC(═O)C(CH₃)OHCH₂OH or —NC(═O)CH(OH)C(CH₃)₂OH. In certain embodiments, one of X and Y is N(C(═O)Rₑ). In some embodiments, X is N(C(═O)Rₑ). In some embodiments, Y is N(C(═O)Rₑ).

In some embodiments, one of X and Y is CH or N and the other of X and Y is NRₑ, N(C(Rₑ)₂C(═O)NHR_{f}), N(C(═O)Rₑ), N(C(═O)ORₑ), NC(═O)C(Rₑ)₃, NC(═O)C(Rₑ)₂(Rₑ)₃, NS(═O)₂Rₑ, NS(═O)₂NRₑRₕ, or NS(O)NRₑRₕ.

In some embodiments, one of X and Y is CH or N and the other of X and Y is —N(alkyl), —N-(hetero)alkyl, —N(heterocycle)-N—CN, —N—CH₂OH, —NC(═O)CH₃, —NCH₂CN, —NS(═O)₂CH₃, —NC(═O)OCH₃, or —NC(═O)OCH₂CH₃, —NC(═O)CH₂OH, —NC(═O)C(CH₃)OH, —NC(═O)C(CH₃)OHCH₂OH or —NC(═O)CH(OH)C(CH₃)₂OH.

In embodiments, Rₑ is alkyl, carbocyclyl, or heterocyclyl. In some embodiments, Rₑ is alkyl. In embodiments, wherein Rₑ is CH₃.

In some embodiments, one of X and Y is CH₂.

In some embodiments, one of X and Y is NRₑ, N(C(Rₑ)₂C(═O)NHR_{f}), N(C(═O)Rₑ), N(C(═O)ORₑ), NC(═O)C(Rₑ)₃, NC(═O)C(Rₑ)₂(Rₑ)₃, NS(═O)₂Rₑ, NS(═O)₂NRₑRₕ, or NS(O)NRₑRₕ, and the other one of X and Y is CH₂.

In some embodiments, one of X and Y is —N(alkyl), —N(heterocycle)-N—CN, —N—CH₂OH, —NC(═O)CH₃, —NCH₂CN, —NS(═O)₂CH₃, —NC(═O)OCH₃, or —NC(═O)OCH₂CH₃, —NC(═O)CH₂OH, —NC(═O)C(CH₃)OH, —NC(═O)C(CH₃)OHCH₂OH or —NC(═O)CH(OH)C(CH₃)₂OH, and the other one of X and Y is CH₂.

In some embodiments, one of X and Y is N(C(═O)Rₑ), and Rₑ is alkyl, carbocyclyl, or heterocyclyl; and the other one of X and Y is CH₂. In certain embodiments, one of X and Y is N(C(═O)CH₃), and the other one of X and Y is CH₂.

In embodiments, one of X and Y is CH or N and the other of X and Y is C(CN), C(Rₑ), or C(C(═O)Rₑ), and Rₑ is (hetero)alkyl, (hetero)alkenyl, (hetero)alkynyl, carbocyclyl, or heterocyclyl.

In embodiments, X is CH or N and Y is C(CN), C(Rₑ), or C(C(═O)Rₑ);

In embodiments, Y is CH or N and X is C(CN), C(Rₑ), or C(C(═O)Rₑ);

In embodiments, one of X and Y is CH and the other one of X and Y is N.

In embodiments, X is CH and Y is N.

In embodiments, Y is CH and X is N.

In embodiments, Rₑ is alkyl. In embodiments, Rₑ is CH₃ or CH₂(CH₃)₂.

In embodiments, Hy is

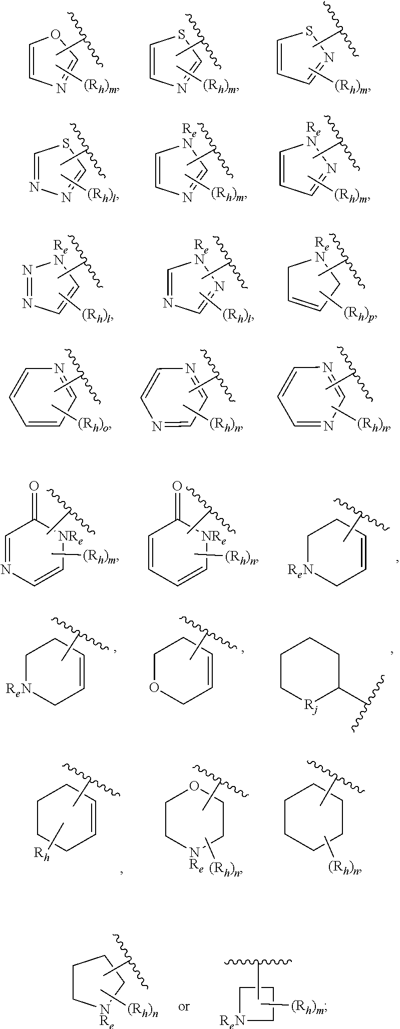

wherein l is 0 or 1; m is 0, 1, or 2; n is 0, 1, 2, or 3; o is 0, 1, 2, 3, or 4; p is 0, 1, 2, 3, 4, or 5, and Rj is O, Sn, NH or N(alkyl).

In embodiments, Hy is

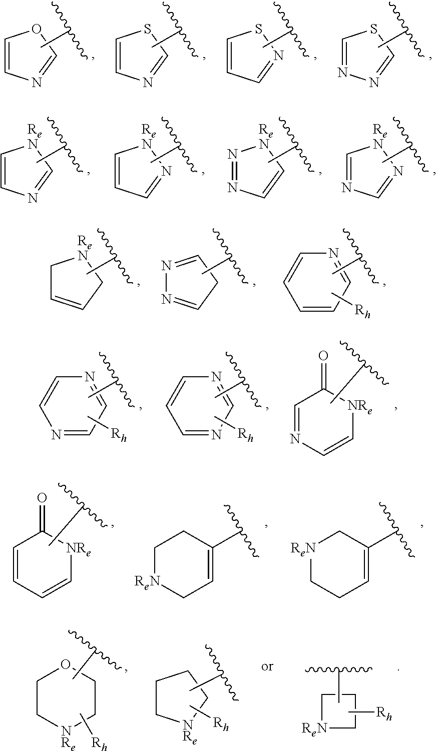

In embodiments, Hy is

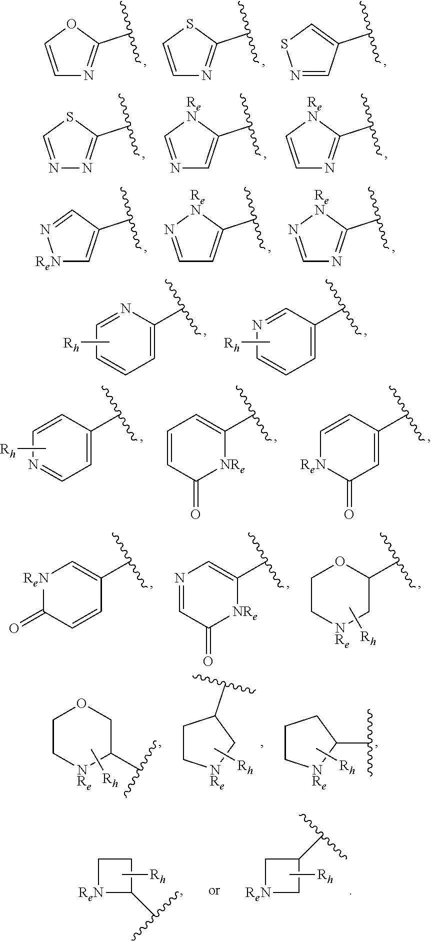

In some embodiments, Hy is

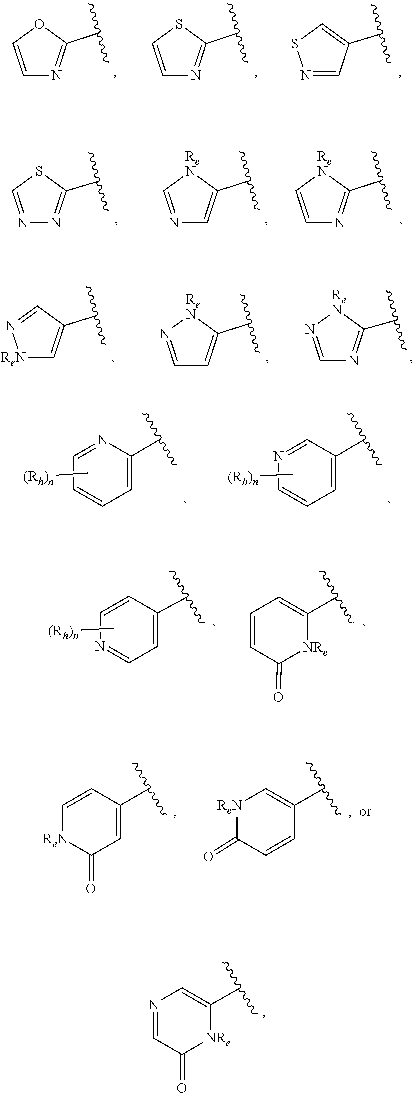

wherein
Rₕ is halogen, OH, alkyl, NH₂, NH(C₁₋₆ alkyl), CN, —CH₂OCH₃, —OCH₃, —OCH₂CH₃, —OC(CH₃)₃, OCHF₂, OCF₃, —C(═O)CH₃, —CH₂C(═O)NHCH₃, —C(═O)OC(CH₃)₃, O-heterocycle, C₃₋₆ cycloalkyl, CH₂CH₂CN, CH₂CN, C(CH₃)₂CN, CH(CH₃)CN, CH₂CF₃, —CH₂OCH₃, CH₂CH₂OCH₃, CH₂CH₂OH, CH(OH)CHF₂, aryl, heterocyclyl,
n is 0, 1, 2, or 3; and
Rₑ is H, OCH₃, CH₃, CH₂CH₃, CH₂F, CHF₂, C₃₋₆ cycloalkyl, —C(═O)CH₃ or heterocyclic.

In embodiments, Hy is

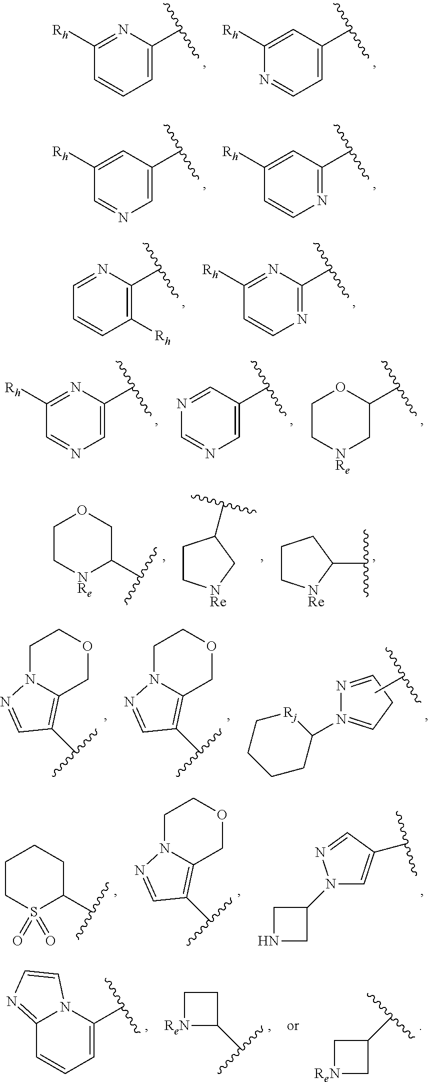

In some embodiments of Hy group, Rₑ is H, OCH₃, CH₃, CH₂CH₃, CH₂F, CHF₂, C₃₋₆ cycloalkyl, —C(═O)CH₃ or heterocyclic. In some embodiments, Rₑ is CH₂F, CHF₂, CH₃, CH₂CH₃, or cyclopropyl. In some embodiments, Rₑ is CH₂F. In some embodiments, Rₑ is CHF₂. In some embodiments of Hy group, Rⱼ is O, S, NH, or N(alkyl).

In some embodiments of Hy group, Rₕ is halogen, OH, alkyl, NH₂, NH(C₁₋₆ alkyl), CN, —CH₂OCH₃, —OCH₃, —OCH₂CH₃, —OC(CH₃)₃, OCHF₂, OCF₃, —C(═O)CH₃, —CH₂C(═O)NHCH₃, —C(═O)OC(CH₃)₃, O-heterocycle, C₃₋₆ cycloalkyl, CH₂CH₂CN, CH₂CN, C(CH₃)₂CN, CH(CH₃)CN, CH₂CF₃, —CH₂OCH₃, CH₂CH₂OCH₃, CH₂CH₂OH, CH(OH)CHF₂, aryl, heterocyclyl or heteroaryl. In some embodiments, Rₕ is

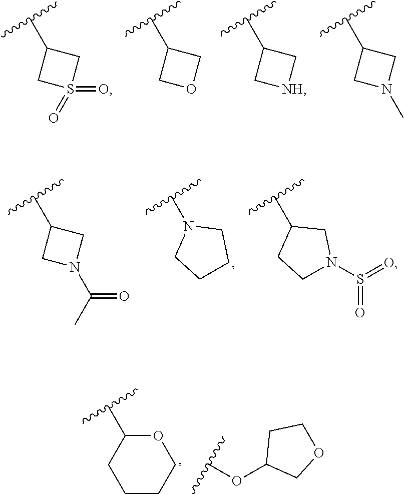

In some embodiments of Hy group, Rⱼ is O, S or NRₑ, wherein Rₑ is H or alkyl.

In embodiments, Rₑ is H. In embodiments, Rₑ is OCH₃. In embodiments, Rₑ is CH₃. In embodiments, Rₑ is —C(═O)CH₃. In embodiments, Rₑ is -heterocyclic. In embodiments, Rₕ is OCH₃. In embodiments, Rₕ is O or NRₑ. In embodiments, Rₕ is halogen.

In embodiments, Hy is selected from:

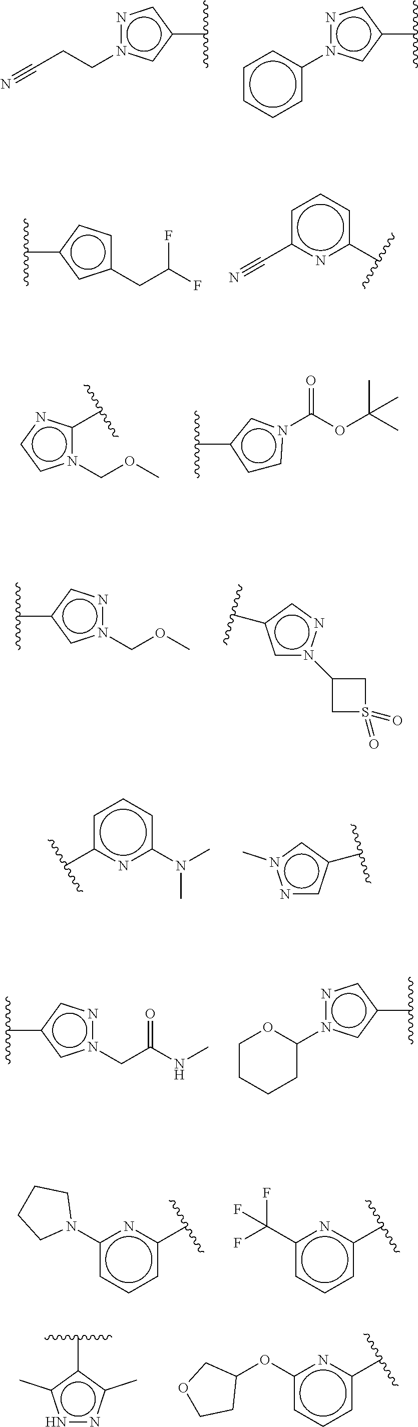

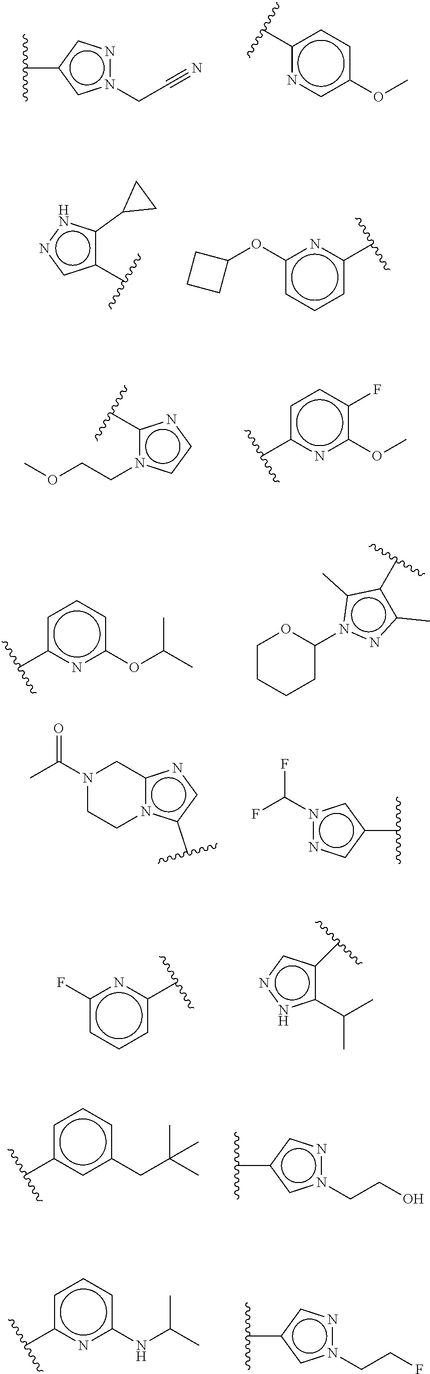

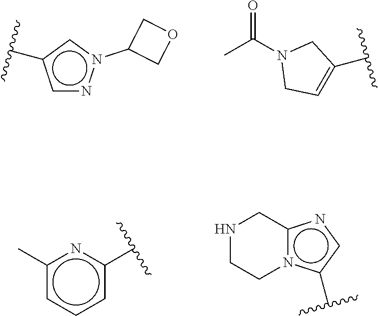

In some embodiments, Cy is -A-L-B.

In some embodiments, Cy is -A=L-B.

In some embodiments, Cy is

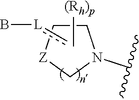

wherein p=0, 1, 2, 3 or 4; n′=0, 1 or 2; Z is C(Rₕ), C(Rₕ)₂, C, N, N(Rₑ), O, S, S(═O), S(═O)₂, Rₕ is H, halogen, alkyl, ═O, alkoxy or CH₂CH₂OCH₃, and Rₑ is H or alkyl, or

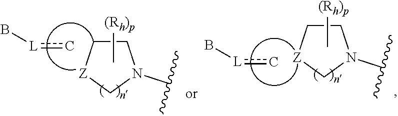

wherein ring C is a heterocycle, p=0, 1, 2, 3 or 4; n′=0, 1 or 2; Z is C(Rₕ), C(Rₕ)₂, C, N, N(Rₑ), O or S.

In some embodiments, Cy is

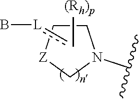

n wherein p=0, 1, 2, 3 or 4; n′=0, 1 or 2; Z is C(Rₕ), C(Rₕ)₂, C, N, N(Rₑ), O, S, S(═O) or S(═O)₂, Rₕ is H, halogen, alkyl, ═O, alkoxy or CH₂CH₂OCH₃, and Rₑ is H or alkyl.

In some embodiments, Cy is

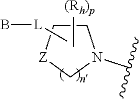

wherein p=0, 1, 2, 3 or 4; n′=0, 1 or 2; Z is C(Rₕ), C(Rₕ)₂, N, N(Rₑ), O, S, S(═O) or S(═O)₂, Rₕ is H, halogen, alkyl, ═O, alkoxy or CH₂CH₂OCH₃, and Rₑ is H or alkyl.

In some embodiments, Cy is

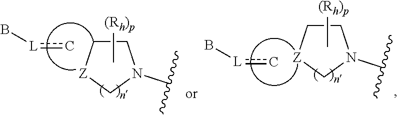

wherein ring C is a heterocycle, p=0, 1, 2, 3 or 4; n′=0, 1 or 2; Z is C(Rₕ), C(Rₕ)₂, C, N, N(Rₑ), O, S, S(═O) or S(═O)₂.

In embodiments, A is

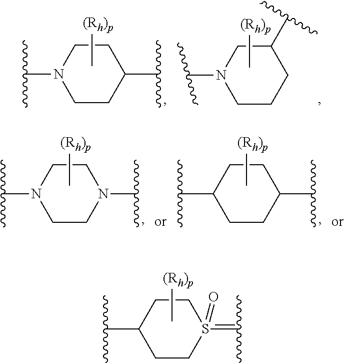

and p is 0, 1, 2, 3, or 4.

In some embodiments, A is

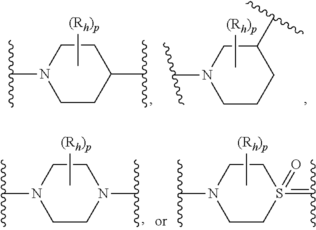

and p is 0, 1, 2, 3, or 4.

In embodiments, p is 0. In embodiments, p is 1

In embodiments, A is

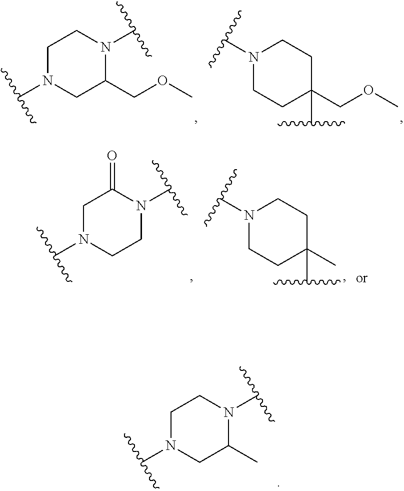

In some embodiments, A is

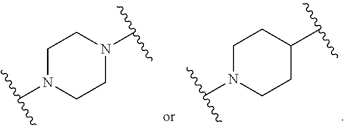 [SMILES: *N1CCN(*)CC1 ; *C1CCN(*)CC1]

In some embodiments, A is

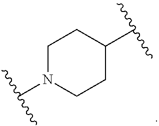 [SMILES: *C1CCN(*)CC1]

In some embodiments, A is

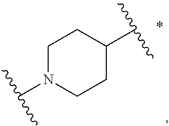 [SMILES: *C1CCN(*)CC1]

wherein the asterisk represents the point of attachment to L.

In embodiments, A is

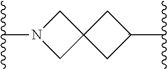 [SMILES: *C1CC2(C1)CN(*)C2]

In embodiments, A is

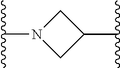 [SMILES: *C1CN(*)C1]

In embodiments, A is

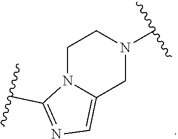 [SMILES: *c1ncc2n1CCN(*)C2]

In some embodiments, -A-L-B is

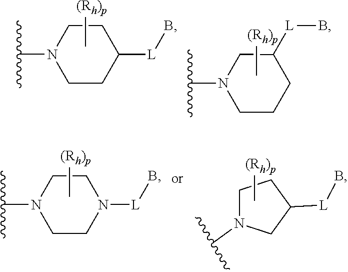

wherein p is 0, 1, 2, 3, or 4, and Rₕ is H, halogen, alkyl, alkoxy, CH₂CH₂OCH₃, or two Rₕ attached to the same carbon together with the atoms to which they are attached form ═O.

In some embodiments, -A-L-B is

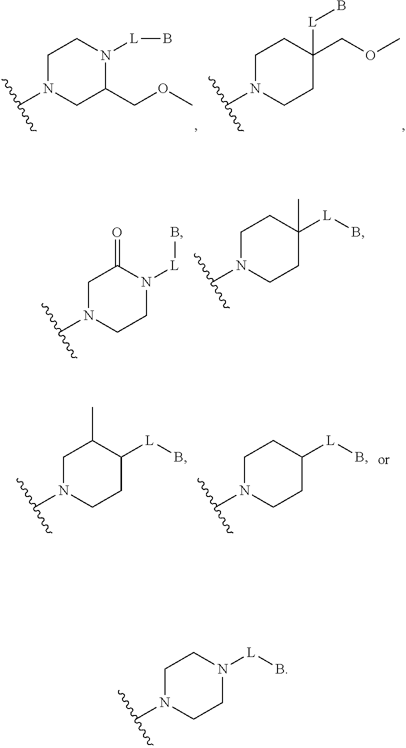

In some embodiments, -A-L-B is

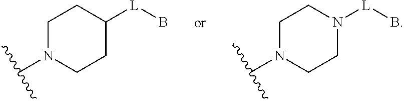

In some embodiments, -A-L-B is

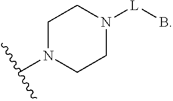

In some embodiments, L is absent, O, C(Rₕ), C(Rₕ)₂, —OC(Rₕ)₂—, C(═O), C(═O)N(Rₑ), NRₑ or N, Rₕ is H, halogen, alkyl, haloalkyl, OH or CH₂OH, and Rₑ is H or alkyl.

In some embodiments, L is O, CH₂, OCH₂, C(═O), NRₑ or N. In some embodiments, L is O, CH₂, CCH₃, CCF₃, CHOH, CHF, CF₂, C(CH₂OH)₂, CH, C(═O), C(═O)N(CH₃), C(═O)NH, N, NH or NCH₃.

In embodiments, L is O, CH₂, C(═O), or NRₑ or N. In some embodiments of group L, Rₑ is CH₃. In some embodiments, L is L is O or, CH₂.

In some embodiments, L is CH₂.

In some embodiments, B is a 5- to 7-membered hetero- or carbocyclic ring.

In embodiments, B is

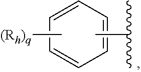

wherein q is an integer of 0, 1, 2, 3, 4, or 5.

In embodiments, B is

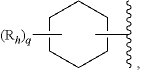

wherein q is an integer of 0, 1, 2, 3, 4, or 5.

In some embodiments, B is

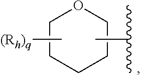

wherein q is 0, 1, 2, 3, 4, or 5.

In embodiments, q is 0, 1, 2 or 3. In certain embodiments, q is 2. In certain embodiments, q is 1.

In certain embodiments, B is

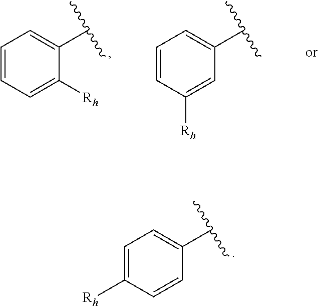

In some embodiments, B is

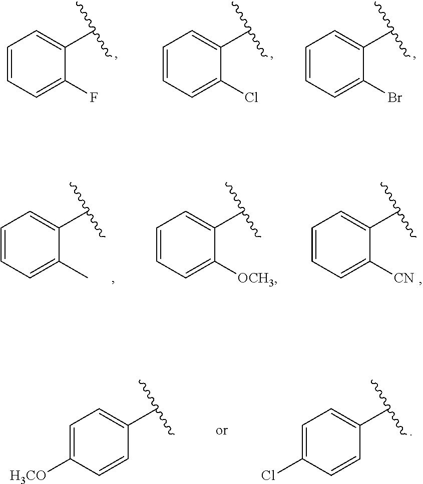

In embodiments, B is

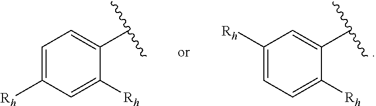

In embodiments, B is

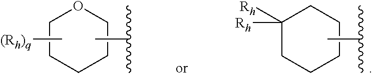

In some embodiments of group B, Rₕ is independently halogen, alkoxy, CN, alkyl, haloalkyl or S(═O)₂C₁₋₆alkyl. In certain embodiments of group B, Rₕ is independently halogen. In certain of these embodiments, each Rₕ is independently F or Cl.

In embodiments, B is

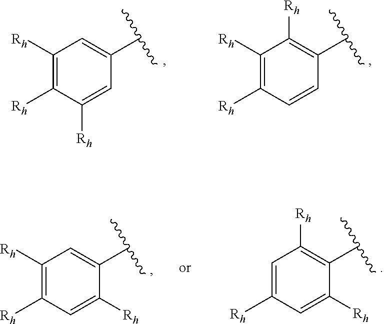

In embodiments, each Rₕ is independently halogen, CN or OMe.

In embodiments, each Rₕ is independently halogen. In embodiments, each Rₕ is independently F or Cl.

In embodiments, B is

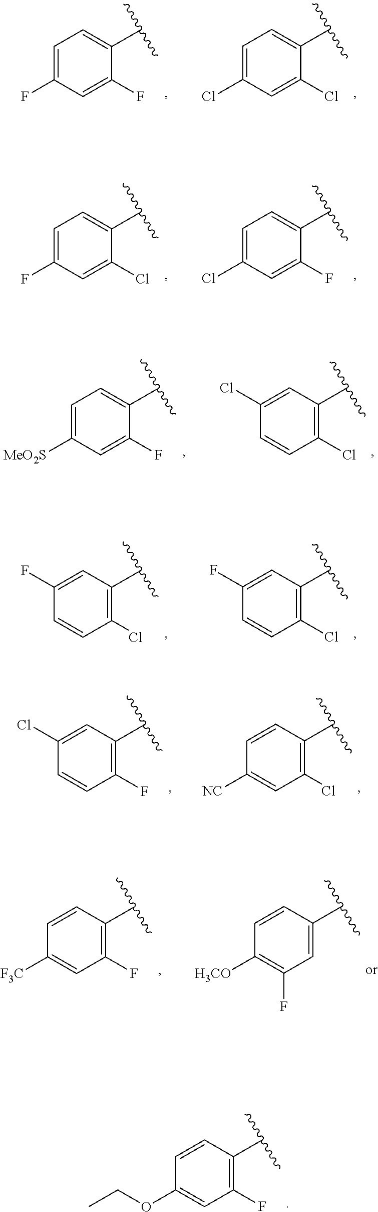

In embodiments, B is

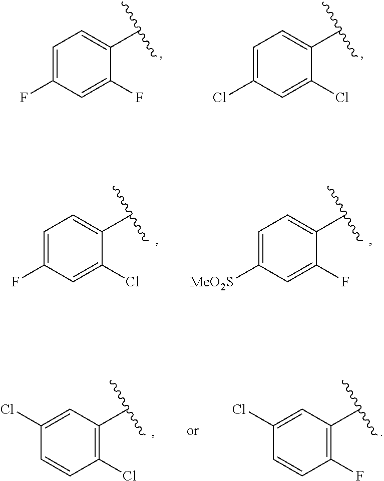 [SMILES: *c1ccc(F)cc1F ; *c1ccc(Cl)cc1Cl ; *c1ccc(F)cc1Cl ; *c1ccc(S(C)(=O)=O)cc1F ; *c1cc(Cl)ccc1Cl ; *c1cc(Cl)ccc1F]

In some embodiments, B is

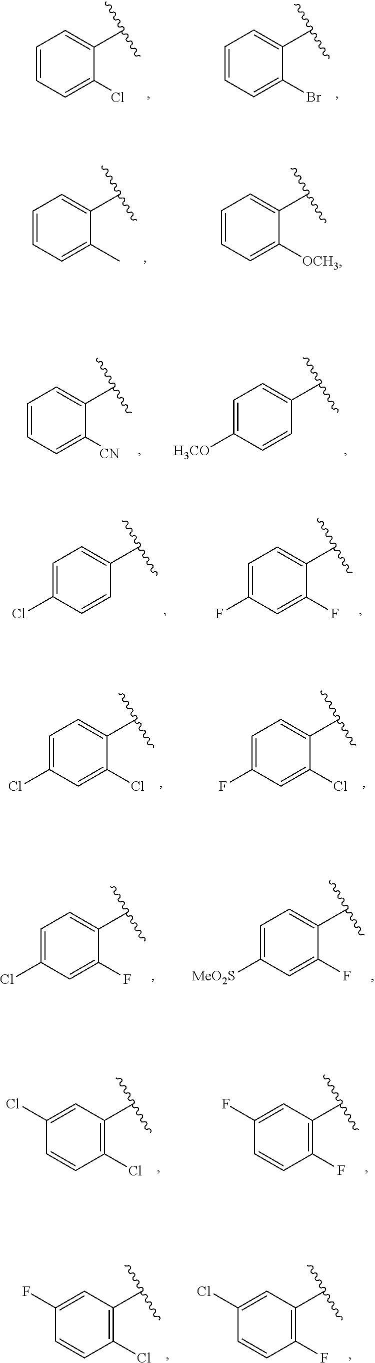

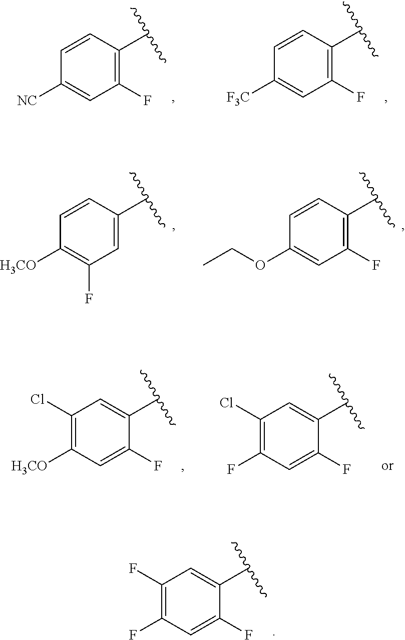

In some embodiments, B is

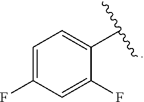 [SMILES: *c1ccc(F)cc1F]

In embodiments, -A-L-B is

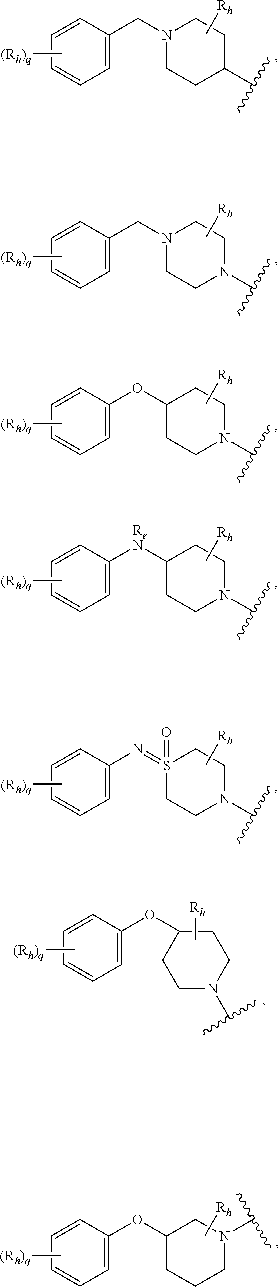

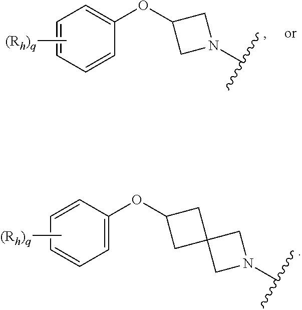

In embodiments, q is 2.

In embodiments, -A-L-B is

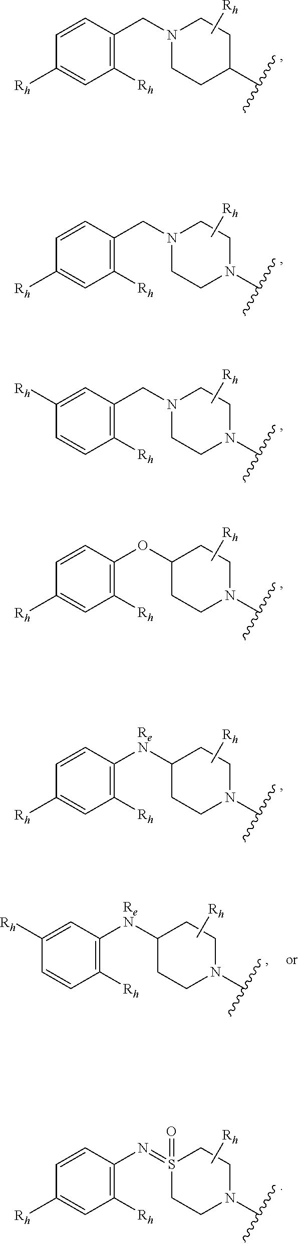

In embodiments, Cy is

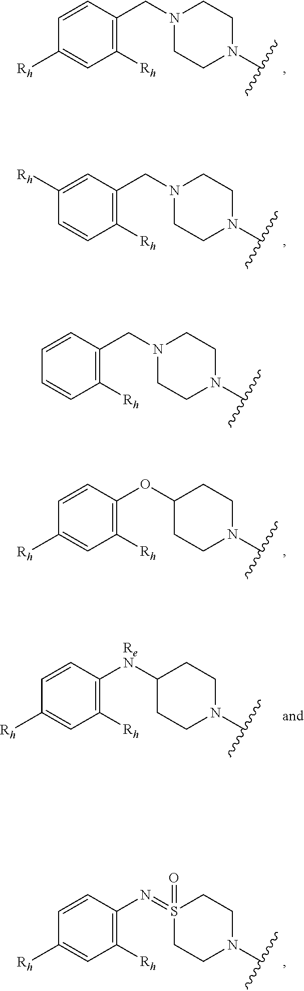

wherein Rₕ is halogen, alkoxy, CN, alkyl, haloalkyl S(═O)₂alkyl, or CH₂CH₂OCH₃.

In embodiments, -A-L-B is

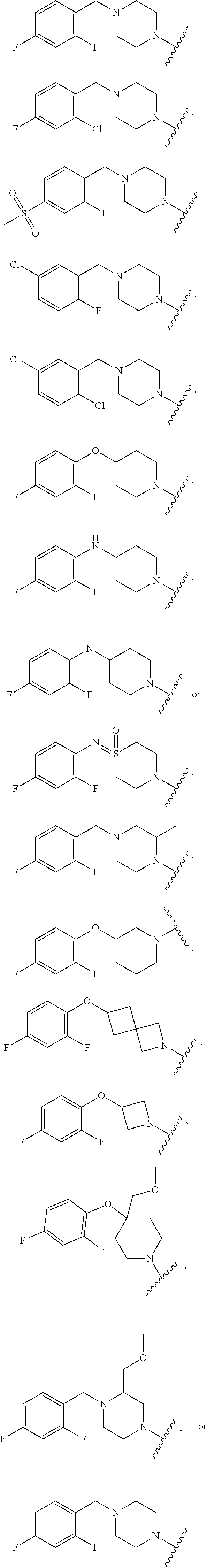

In some embodiments, Cy is

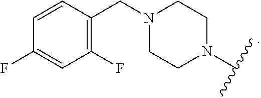 [SMILES: *N1CCN(Cc2ccc(F)cc2F)CC1]

In embodiments, provided herein is a compound selected from the group:

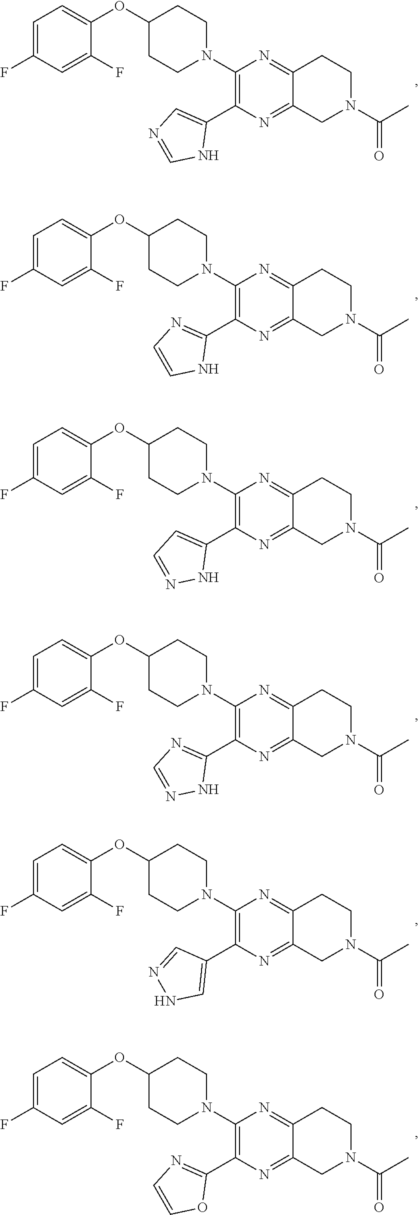 [SMILES: CC(=O)N1CCc2nc(N3CCC(Oc4ccc(F)cc4F)CC3)c(-c3cnc[nH]3)nc2C1 ; CC(=O)N1CCc2nc(N3CCC(Oc4ccc(F)cc4F)CC3)c(-c3ncc[nH]3)nc2C1 ; CC(=O)N1CCc2nc(N3CCC(Oc4ccc(F)cc4F)CC3)c(-c3ccn[nH]3)nc2C1 ; CC(=O)N1CCc2nc(N3CCC(Oc4ccc(F)cc4F)CC3)c(-c3ncn[nH]3)nc2C1 ; CC(=O)N1CCc2nc(N3CCC(Oc4ccc(F)cc4F)CC3)c(-c3cn[nH]c3)nc2C1 ; CC(=O)N1CCc2nc(N3CCC(Oc4ccc(F)cc4F)CC3)c(-c3ncco3)nc2C1]

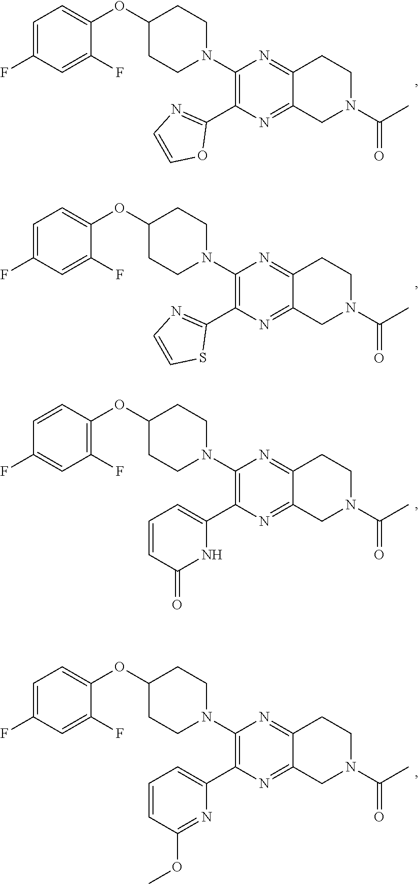

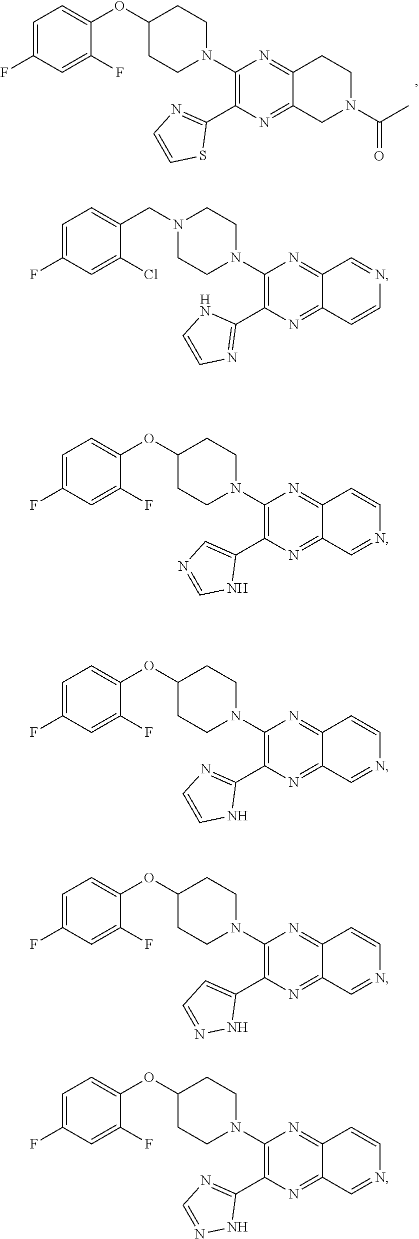

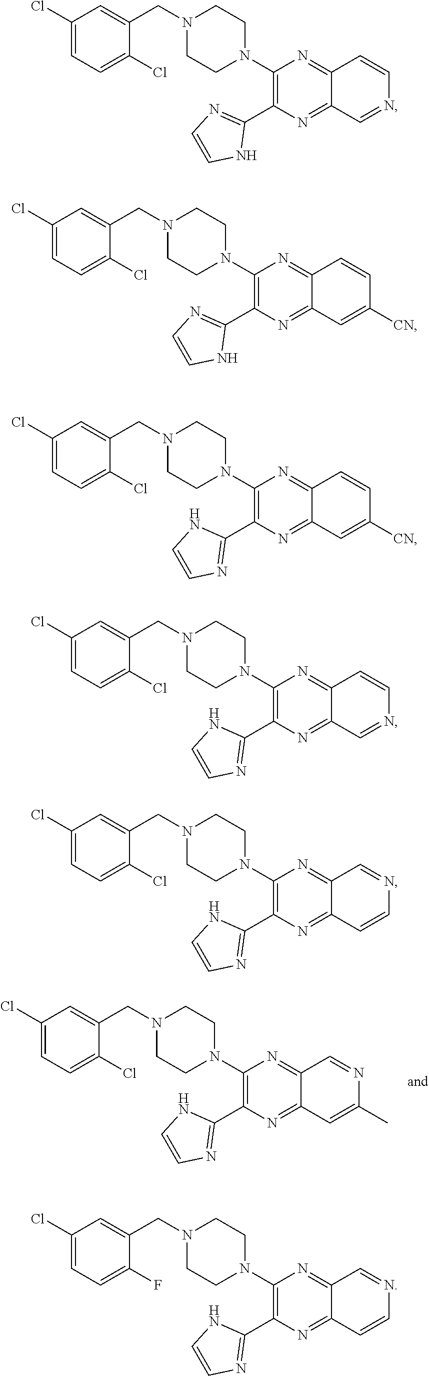

In embodiments, provides herein is a compound selected from:

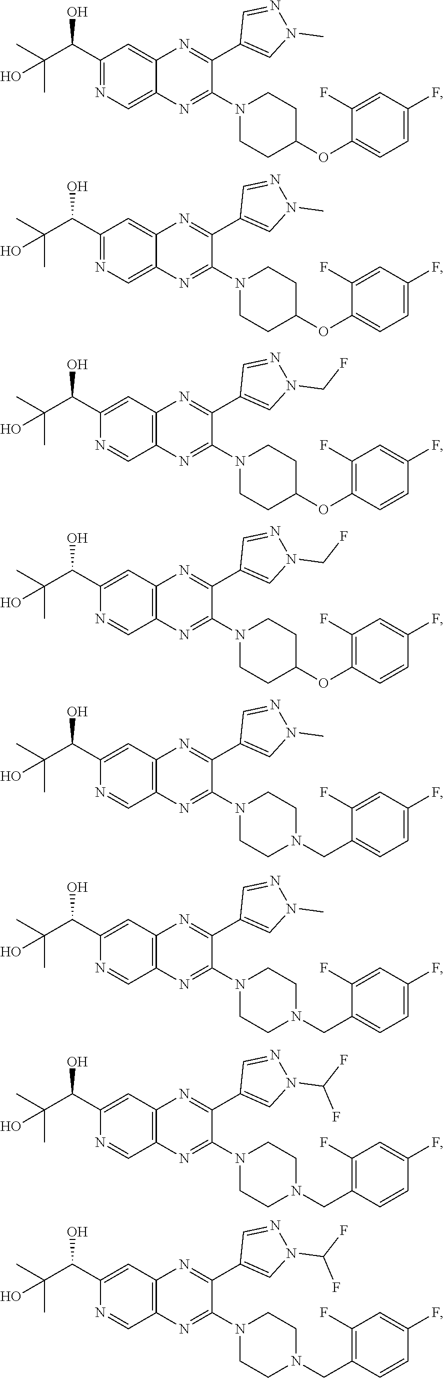

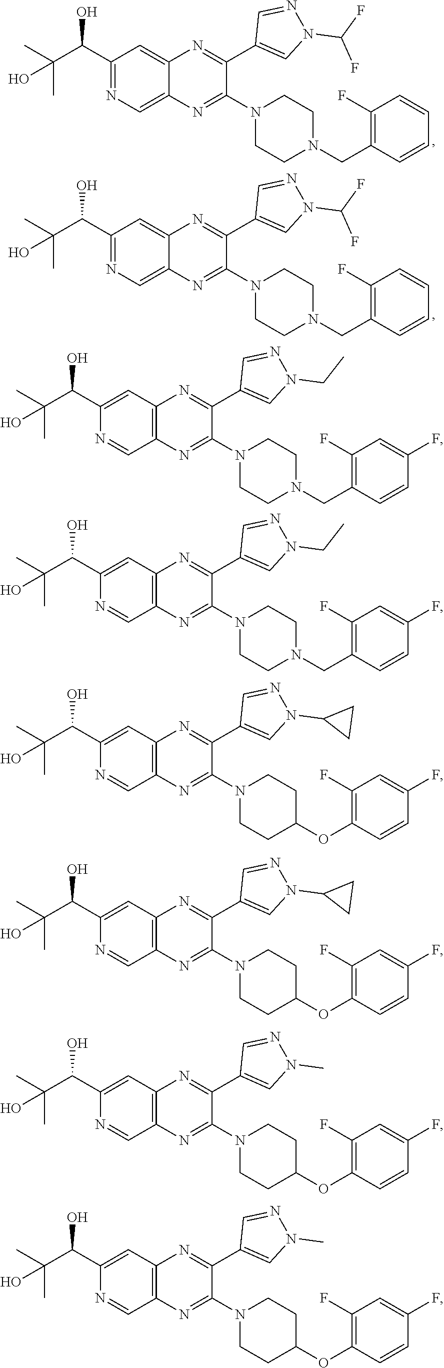

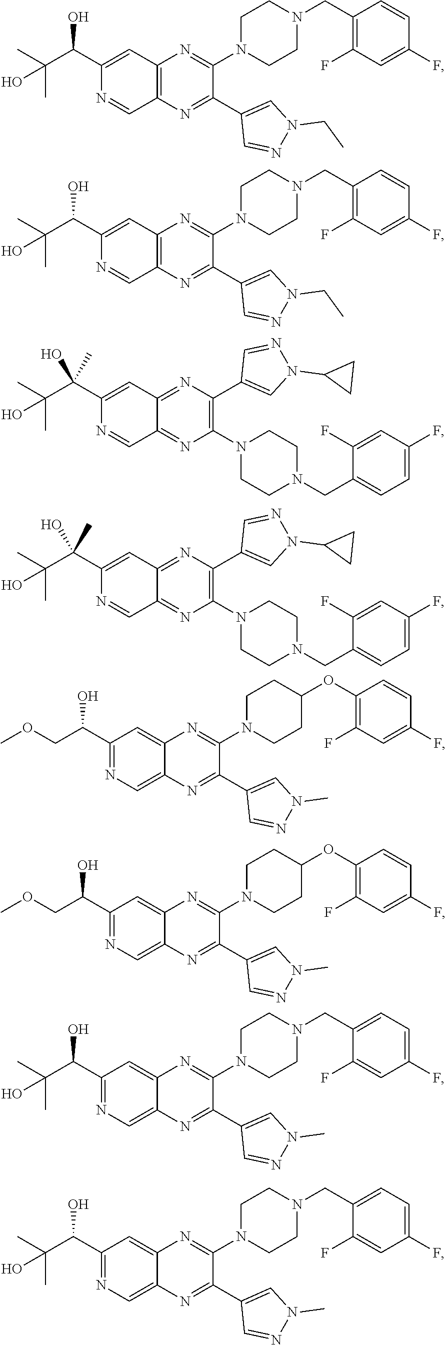

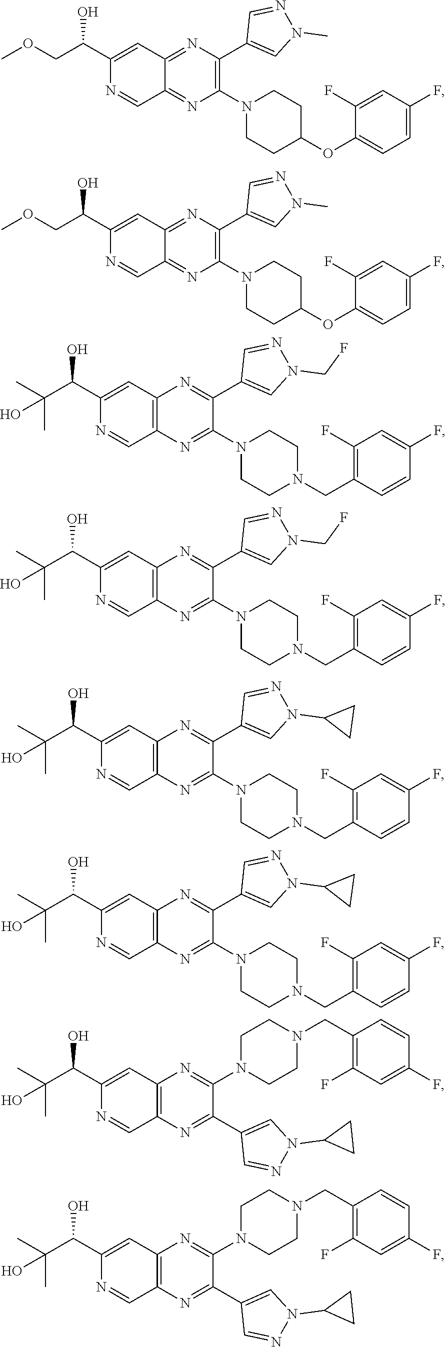

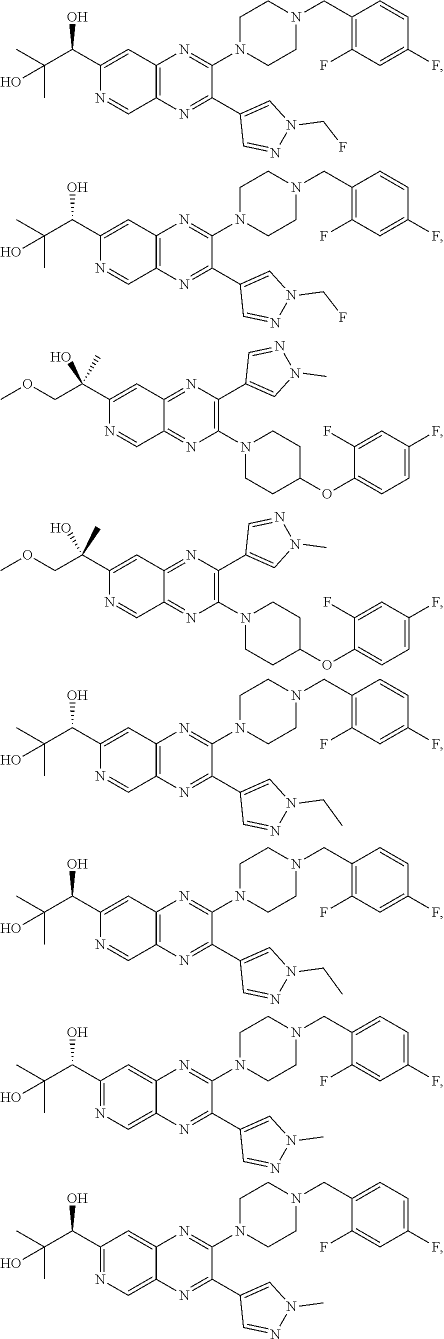

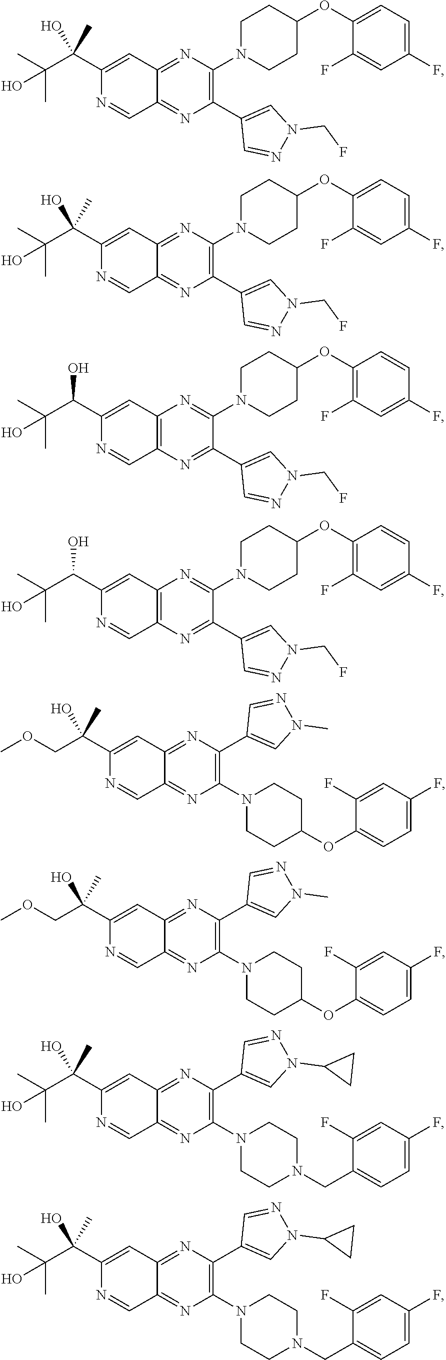

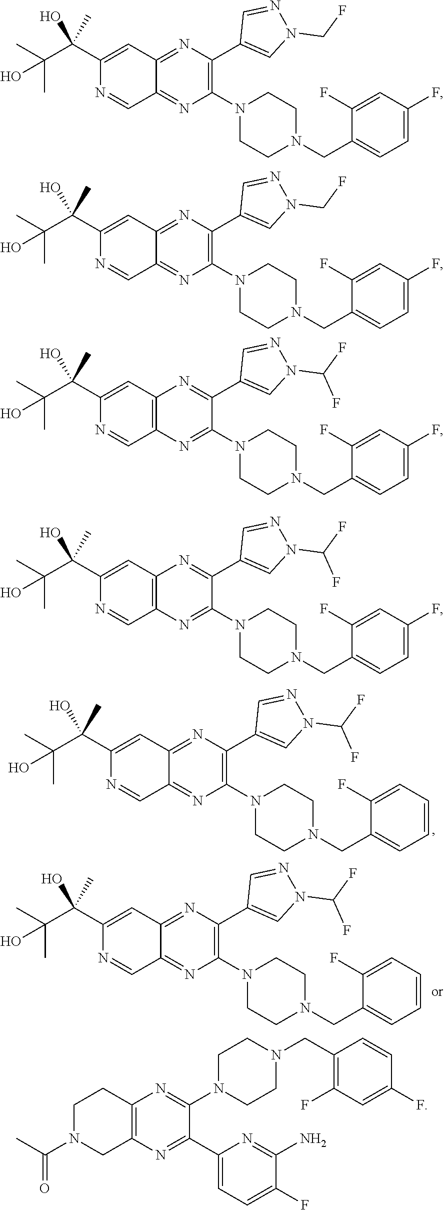

In embodiments, provided herein is compound selected from the group consisting of:

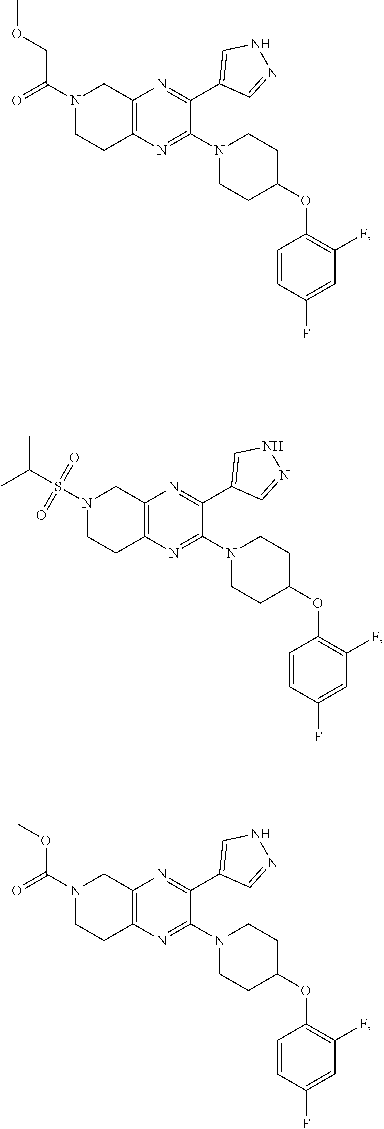 [SMILES: COCC(=O)N1CCc2nc(N3CCC(Oc4ccc(F)cc4F)CC3)c(-c3cn[nH]c3)nc2C1 ; CC(C)S(=O)(=O)N1CCc2nc(N3CCC(Oc4ccc(F)cc4F)CC3)c(-c3cn[nH]c3)nc2C1 ; COC(=O)N1CCc2nc(N3CCC(Oc4ccc(F)cc4F)CC3)c(-c3cn[nH]c3)nc2C1]

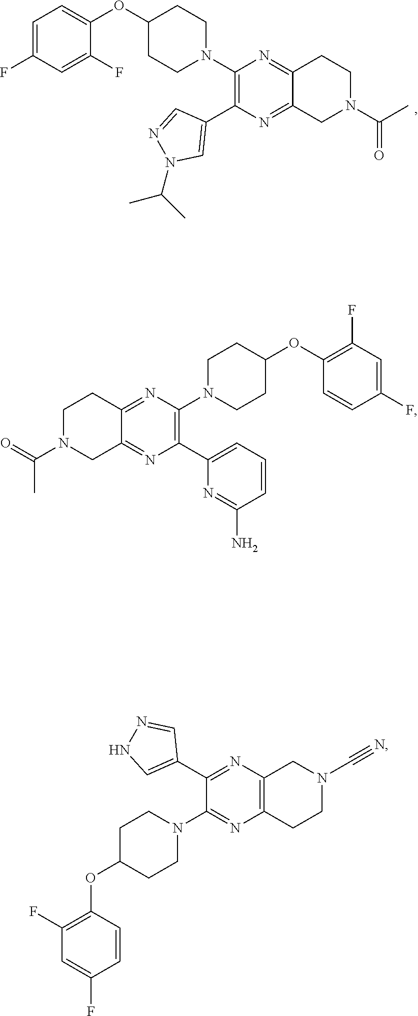 [SMILES: CC(=O)N1CCc2nc(N3CCC(Oc4ccc(F)cc4F)CC3)c(-c3cnn(C(C)C)c3)nc2C1 ; CC(=O)N1CCc2nc(N3CCC(Oc4ccc(F)cc4F)CC3)c(-c3cccc(N)n3)nc2C1 ; N#CN1CCc2nc(N3CCC(Oc4ccc(F)cc4F)CC3)c(-c3cn[nH]c3)nc2C1]

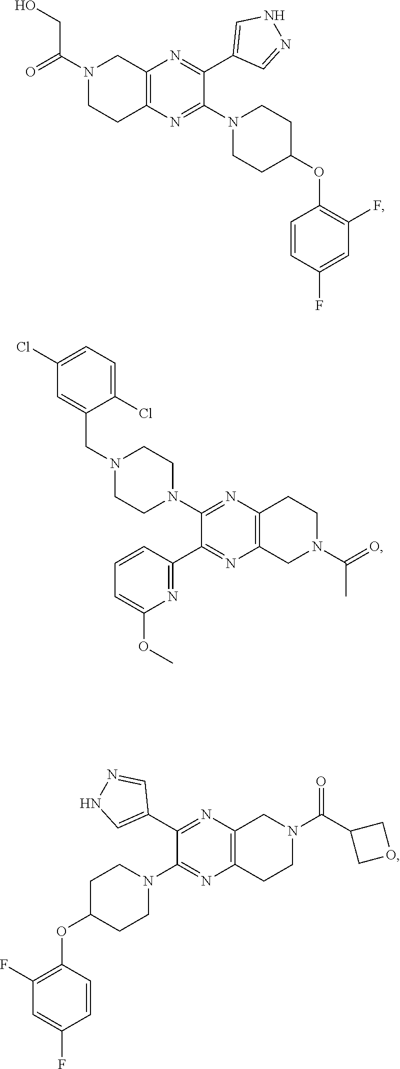 [SMILES: O=C(CO)N1CCc2nc(N3CCC(Oc4ccc(F)cc4F)CC3)c(-c3cn[nH]c3)nc2C1 ; COc1cccc(-c2nc3c(nc2N2CCN(Cc4cc(Cl)ccc4Cl)CC2)CCN(C(C)=O)C3)n1 ; O=C(C1COC1)N1CCc2nc(N3CCC(Oc4ccc(F)cc4F)CC3)c(-c3cn[nH]c3)nc2C1]

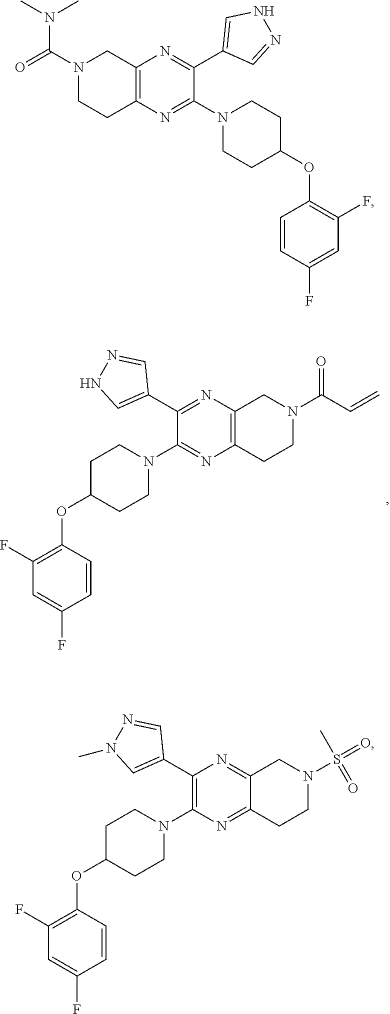 [SMILES: CN(C)C(=O)N1CCc2nc(N3CCC(Oc4ccc(F)cc4F)CC3)c(-c3cn[nH]c3)nc2C1 ; C=CC(=O)N1CCc2nc(N3CCC(Oc4ccc(F)cc4F)CC3)c(-c3cn[nH]c3)nc2C1 ; Cn1cc(-c2nc3c(nc2N2CCC(Oc4ccc(F)cc4F)CC2)CCN(S(C)(=O)=O)C3)cn1]

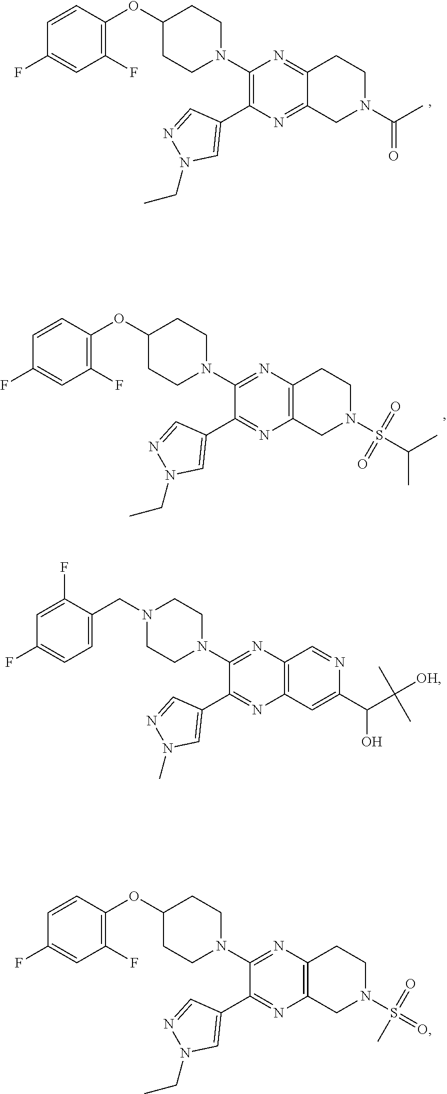 [SMILES: CCn1cc(-c2nc3c(nc2N2CCC(Oc4ccc(F)cc4F)CC2)CCN(C(C)=O)C3)cn1 ; CCn1cc(-c2nc3c(nc2N2CCC(Oc4ccc(F)cc4F)CC2)CCN(S(=O)(=O)C(C)C)C3)cn1 ; Cn1cc(-c2nc3cc(C(O)C(C)(C)O)ncc3nc2N2CCN(Cc3ccc(F)cc3F)CC2)cn1 ; CCn1cc(-c2nc3c(nc2N2CCC(Oc4ccc(F)cc4F)CC2)CCN(S(C)(=O)=O)C3)cn1]

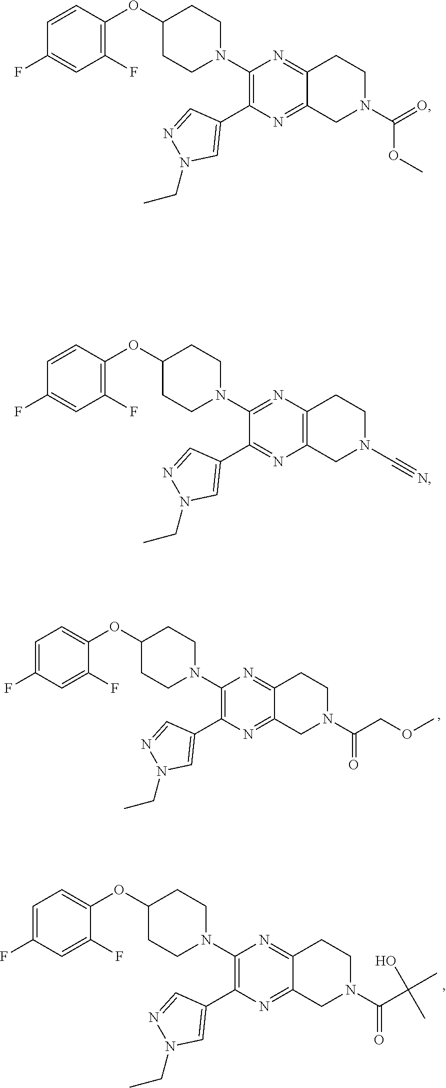 [SMILES: CCn1cc(-c2nc3c(nc2N2CCC(Oc4ccc(F)cc4F)CC2)CCN(C(=O)OC)C3)cn1 ; CCn1cc(-c2nc3c(nc2N2CCC(Oc4ccc(F)cc4F)CC2)CCN(C#N)C3)cn1 ; CCn1cc(-c2nc3c(nc2N2CCC(Oc4ccc(F)cc4F)CC2)CCN(C(=O)COC)C3)cn1 ; CCn1cc(-c2nc3c(nc2N2CCC(Oc4ccc(F)cc4F)CC2)CCN(C(=O)C(C)(C)O)C3)cn1]

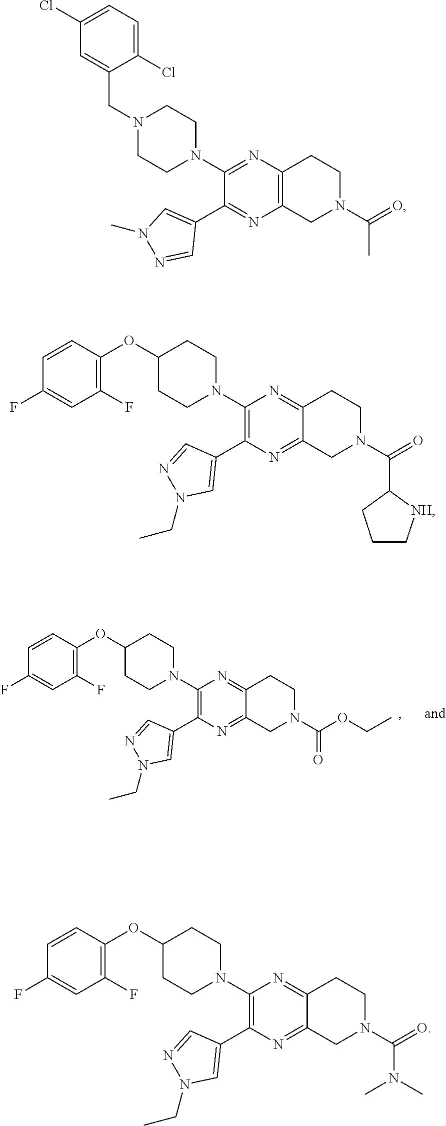 [SMILES: CC(=O)N1CCc2nc(N3CCN(Cc4cc(Cl)ccc4Cl)CC3)c(-c3cnn(C)c3)nc2C1 ; CCn1cc(-c2nc3c(nc2N2CCC(Oc4ccc(F)cc4F)CC2)CCN(C(=O)C2CCCN2)C3)cn1 ; CCOC(=O)N1CCc2nc(N3CCC(Oc4ccc(F)cc4F)CC3)c(-c3cnn(CC)c3)nc2C1 ; CCn1cc(-c2nc3c(nc2N2CCC(Oc4ccc(F)cc4F)CC2)CCN(C(=O)N(C)C)C3)cn1]

In embodiments, provided herein is a compound selected from Table 1, or a pharmaceutically acceptable salt thereof.

TABLE 1
Compounds

| | | | Concat |
|---|---|---|---|
| Cmpd | | | Optical |
| No. | Structure | Name | Rotation^{[1]} |
| 1 | 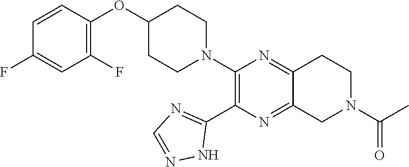 [SMILES: CC(=O)N1CCc2nc(N3CCC(Oc4ccc(F)cc4F)CC3)c(-c3ncn[nH]3)nc2C1] | 1-{2-[4-(2,4- difluorophenoxy)piperidin- 1-yl]-3-(1H-1,2,4-triazol- 5-yl)-5H,6H,7H,8H- pyrido[3,4-b]pyrazin-6- yl}ethan-1-one | NA |
| 2 | 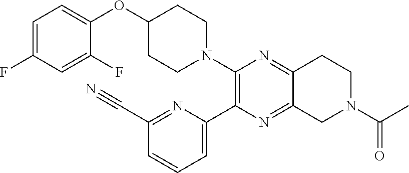 [SMILES: CC(=O)N1CCc2nc(N3CCC(Oc4ccc(F)cc4F)CC3)c(-c3cccc(C#N)n3)nc2C1] | 6-{6-acetyl-2-[4-(2,4- difluorophenoxy)piperidin- 1-yl]-5H,6H,7H,8H- pyrido[3,4-b]pyrazin-3- yl}pyridine-2-carbonitrile | NA |
| 3 | 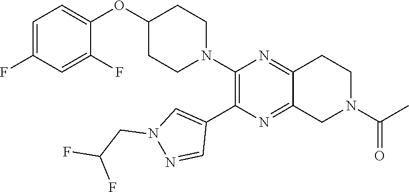 [SMILES: CC(=O)N1CCc2nc(N3CCC(Oc4ccc(F)cc4F)CC3)c(-c3cnn(CC(F)F)c3)nc2C1] | 1-{3-[1-(2,2- difluoroethyl)-1H-pyrazol- 4-yl]-2-[4-(2,4- difluorophenoxy)piperidin- 1-yl]-5H,6H,7H,8H- pyrido[3,4-b]pyrazin-6- yl}ethan-1-one | NA |
| 4 | 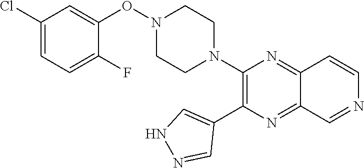 [SMILES: Fc1ccc(Cl)cc1ON1CCN(c2nc3ccncc3nc2-c2cn[nH]c2)CC1] | 1-[(5-chloro-2- fluorophenyl)methyl]-4- [3-(1H-pyrazol-4- yl)pyrido[3,4-b]pyrazin-2- yl]piperazine | NA |
| 5 | 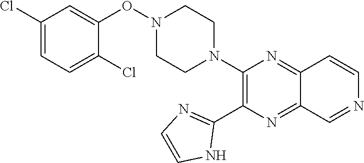 [SMILES: Clc1ccc(Cl)c(ON2CCN(c3nc4ccncc4nc3-c3ncc[nH]3)CC2)c1] | 1-[(2,5- dichlorophenyl)methyl]-4- [3-(1H-imidazol-2- yl)pyrido[3,4-b]pyrazin-2- yl]piperazine | NA |
| 6 | 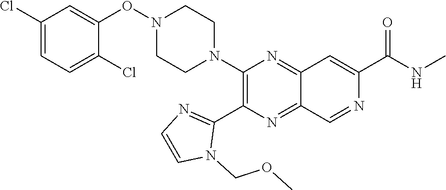 [SMILES: CNC(=O)c1cc2nc(N3CCN(Oc4cc(Cl)ccc4Cl)CC3)c(-c3nccn3COC)nc2cn1] | 3-{4-[(2,5- dichlorophenyl)methyl] piperazin-1-yl}-2-[1- (methoxymethyl)-1H- imidazol-2-yl]-N- methylquinoxaline-6- carboxamide | NA |
| 7 | 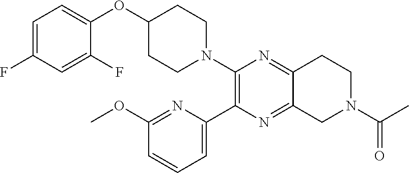 [SMILES: COc1cccc(-c2nc3c(nc2N2CCC(Oc4ccc(F)cc4F)CC2)CCN(C(C)=O)C3)n1] | 1-{2-[4-(2,4- difluorophenoxy)piperidin- 1-yl]-3-(4- methoxypyridin-2-yl)- 5H,6H,7H,8H-pyrido[3,4- b]pyrazin-6-yl}ethan-1- one | NA |
| 8 | 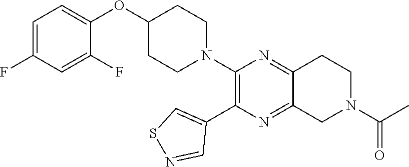 [SMILES: CC(=O)N1CCc2nc(N3CCC(Oc4ccc(F)cc4F)CC3)c(-c3cnsc3)nc2C1] | 1-{2-[4-(2,4- difluorophenoxy)piperidin- 1-yl]-3-(1,2-thiazol-4-yl)- 5H,6H,7H,8H-pyrido[3,4- b]pyrazin-6-yl}ethan-1- one | NA |
| 9 | 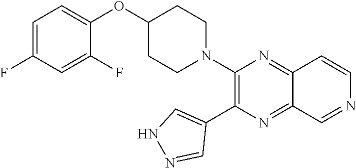 [SMILES: Fc1ccc(OC2CCN(c3nc4ccncc4nc3-c3cn[nH]c3)CC2)c(F)c1] | 4-(2,4-difluorophenoxy)- 1-[3-(1H-pyrazol-4- yl)pyrido[3,4-b]pyrazin-2- yl]piperidine | NA |
| 10 | 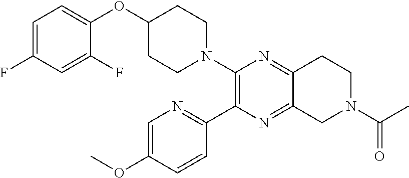 [SMILES: COc1ccc(-c2nc3c(nc2N2CCC(Oc4ccc(F)cc4F)CC2)CCN(C(C)=O)C3)nc1] | 1-{2-[4-(2,4- difluorophenoxy)piperidin- 1-yl]-3-(5- methoxypyridin-2-yl)- 5H,6H,7H,8H-pyrido[3,4- b]pyrazin-6-yl}ethan-1- one | NA |
| 11 | 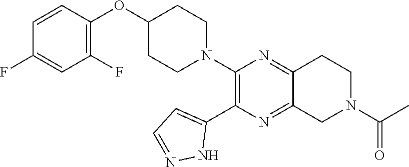 [SMILES: CC(=O)N1CCc2nc(N3CCC(Oc4ccc(F)cc4F)CC3)c(-c3ccn[nH]3)nc2C1] | 1-{2-[4-(2,4- difluorophenoxy)piperidin- 1-yl]-3-(1H-pyrazol-5-yl)- 5H,6H,7H,8H-pyrido[3,4- b]pyrazin-6-yl}ethan-1- one | NA |
| 12 | 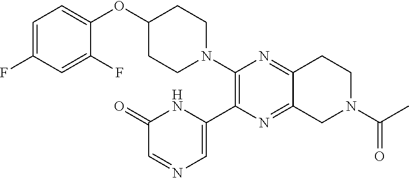 [SMILES: CC(=O)N1CCc2nc(N3CCC(Oc4ccc(F)cc4F)CC3)c(-c3cncc(=O)[nH]3)nc2C1] | 6-{6-acetyl-2-[4-(2,4- difluorophenoxy)piperidin- 1-yl]-5H,6H,7H,8H- pyrido[3,4-b]pyrazin-3- yl}-1,2-dihydropyrazin-2- one | NA |
| 13 | 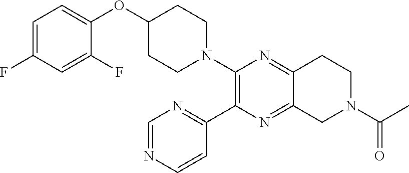 [SMILES: CC(=O)N1CCc2nc(N3CCC(Oc4ccc(F)cc4F)CC3)c(-c3ccncn3)nc2C1] | 1-{2-[4-(2,4- difluorophenoxy)piperidin- 1-yl]-3-(pyrimidin-4-yl)- 5H,6H,7H,8H-pyrido[3,4- b]pyrazin-6-yl}ethan-1- one | NA |
| 14 | 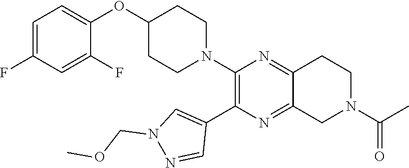 [SMILES: COCn1cc(-c2nc3c(nc2N2CCC(Oc4ccc(F)cc4F)CC2)CCN(C(C)=O)C3)cn1] | 1-{2-[4-(2,4- difluorophenoxy)piperidin- 1-yl]-3-[1- (methoxymethyl)-1H- pyrazol-4-yl]- 5H,6H,7H,8H-pyrido[3,4- b]pyrazin-6-yl}ethan-1- one | NA |
| 15 | 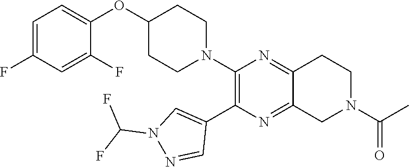 [SMILES: CC(=O)N1CCc2nc(N3CCC(Oc4ccc(F)cc4F)CC3)c(-c3cnn(C(F)F)c3)nc2C1] | 1-{3-[1-(difluoromethyl)- 1H-pyrazol-4-yl]-2-[4- (2,4- difluorophenoxy)piperidin- 1-yl]-5H,6H,7H,8H- pyrido[3,4-b]pyrazin-6- yl}ethan-1-one | NA |
| 16 | 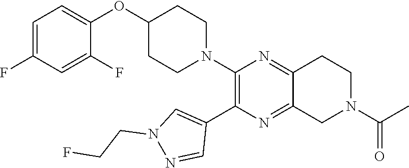 [SMILES: CC(=O)N1CCc2nc(N3CCC(Oc4ccc(F)cc4F)CC3)c(-c3cnn(CCF)c3)nc2C1] | 1-{2-[4-(2,4- difluorophenoxy)piperidin- 1-yl]-3-[1-(2- fluoroethyl)-1H-pyrazol-4- yl]-5H,6H,7H,8H- pyrido[3,4-b]pyrazin-6- yl}ethan-1-one | NA |
| 17 | 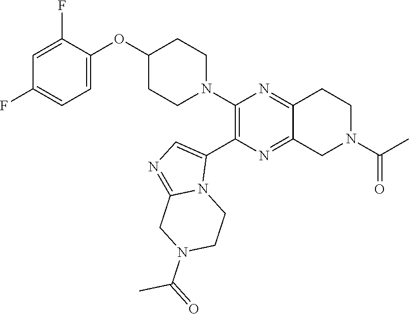 [SMILES: CC(=O)N1CCc2nc(N3CCC(Oc4ccc(F)cc4F)CC3)c(-c3cnc4n3CCN(C(C)=O)C4)nc2C1] | 1-(3-{6-acetyl-2-[4-(2,4- difluorophenoxy)piperidin- 1-yl]-5H,6H,7H,8H- pyrido[3,4-b]pyrazin-3- yl}-5H,6H,7H,8H- imidazo[1,2-a]pyrazin-7- yl)ethan-1-one | NA |
| 18 | 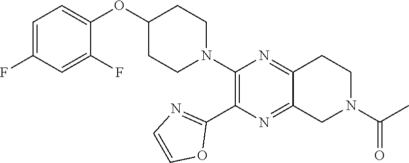 [SMILES: CC(=O)N1CCc2nc(N3CCC(Oc4ccc(F)cc4F)CC3)c(-c3ncco3)nc2C1] | 1-{2-[4-(2,4- difluorophenoxy)piperidin- 1-yl]-3-(1,3-oxazol-2-yl)- 5H,6H,7H,8H-pyrido[3,4- b]pyrazin-6-yl}ethan-1- one | NA |
| 19 | 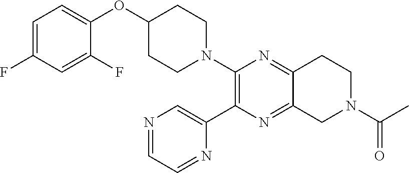 [SMILES: CC(=O)N1CCc2nc(N3CCC(Oc4ccc(F)cc4F)CC3)c(-c3cnccn3)nc2C1] | 1-{2-[4-(2,4- difluorophenoxy)piperidin- 1-yl]-3-(pyrazin-2-yl)- 5H,6H,7H,8H-pyrido[3,4- b]pyrazin-6-yl}ethan-1- one | NA |
| 20 | 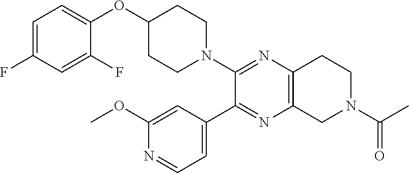 [SMILES: COc1cc(-c2nc3c(nc2N2CCC(Oc4ccc(F)cc4F)CC2)CCN(C(C)=O)C3)ccn1] | 1-{2-[4-(2,4- difluorophenoxy)piperidin- 1-yl]-3-(2- methoxypyridin-4-yl)- 5H,6H,7H,8H-pyrido[3,4- b]pyrazin-6-yl}ethan-1- one | NA |
| 21 | 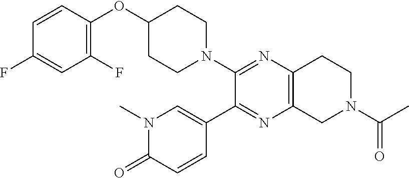 [SMILES: CC(=O)N1CCc2nc(N3CCC(Oc4ccc(F)cc4F)CC3)c(-c3ccc(=O)n(C)c3)nc2C1] | 5-{6-acetyl-2-[4-(2,4- difluorophenoxy)piperidin- 1-yl]-5H,6H,7H,8H- pyrido[3,4-b]pyrazin-3- yl}-1-methyl-1,2- dihydropyridin-2-one | NA |
| 22 | 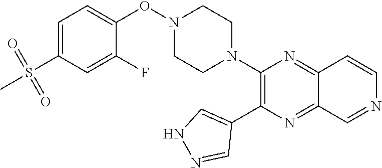 [SMILES: CS(=O)(=O)c1ccc(ON2CCN(c3nc4ccncc4nc3-c3cn[nH]c3)CC2)c(F)c1] | 1-[(2-fluoro-4- methanesulfonylphenyl)m ethyl]-4-[3-(1H-pyrazol-4- yl)pyrido[3,4-b]pyrazin-2- yl]piperazine | NA |
| 23 | 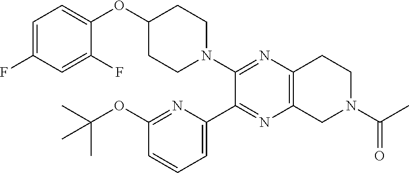 [SMILES: CC(=O)N1CCc2nc(N3CCC(Oc4ccc(F)cc4F)CC3)c(-c3cccc(OC(C)(C)C)n3)nc2C1] | 1-{3-[6-(tert- butoxy)pyridin-2-yl]-2-[4- (2,4- difluorophenoxy)piperidin- 1-yl]-5H,6H,7H,8H- pyrido[3,4-b]pyrazin-6- yl}ethan-1-one | NA |
| 24 | 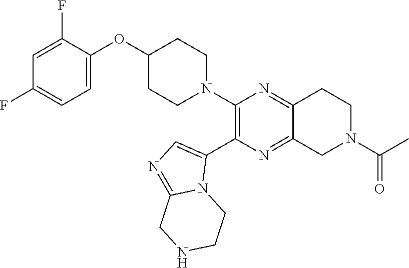 [SMILES: CC(=O)N1CCc2nc(N3CCC(Oc4ccc(F)cc4F)CC3)c(-c3cnc4n3CCNC4)nc2C1] | 1-{2-[4-(2,4- difluorophenoxy)piperidin- 1-yl]-3-{5H,6H,7H,8H- imidazo[1,2-a]pyrazin-3- yl}-5H,6H,7H,8H- pyrido[3,4-b]pyrazin-6- yl}ethan-1-one | NA |
| 25 | 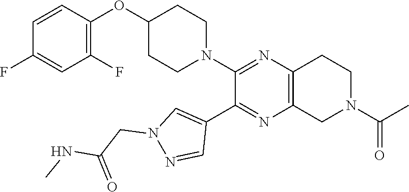 [SMILES: CNC(=O)Cn1cc(-c2nc3c(nc2N2CCC(Oc4ccc(F)cc4F)CC2)CCN(C(C)=O)C3)cn1] | 2-(4-{6-acetyl-2-[4-(2,4- difluorophenoxy)piperidin- 1-yl]-5H,6H,7H,8H- pyrido[3,4-b]pyrazin-3- yl}-1H-pyrazol-1-yl)-N- methylacetamide | NA |
| 26 | 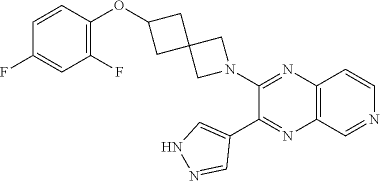 [SMILES: Fc1ccc(OC2CC3(C2)CN(c2nc4ccncc4nc2-c2cn[nH]c2)C3)c(F)c1] | 6-(2,4-difluorophenoxy)- 2-[3-(1H-pyrazol-4- yl)pyrido[3,4-b]pyrazin-2- yl]-2-azaspiro[3.3]heptane | NA |
| 27 | 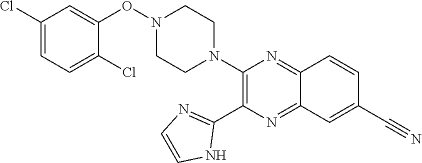 [SMILES: N#Cc1ccc2nc(N3CCN(Oc4cc(Cl)ccc4Cl)CC3)c(-c3ncc[nH]3)nc2c1] | 2-{4-[(2,5- dichlorophenyl)methyl] piperazin-1-yl}-3-(1H- imidazol-2-yl)quinoxaline- 6-carbonitrile | NA |
| 28 | 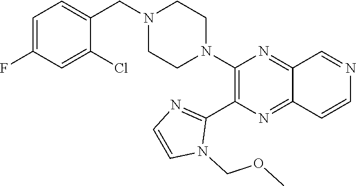 [SMILES: COCn1ccnc1-c1nc2ccncc2nc1N1CCN(Cc2ccc(F)cc2Cl)CC1] | 1-[(2-chloro-4- fluorophenyl)methyl]-4- {2-[1-(methoxymethyl)- 1H-imidazol-2- yl]pyrido[3,4-b]pyrazin-3- yl}piperazine | NA |
| 29 | 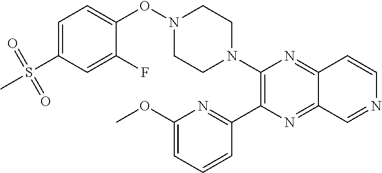 [SMILES: COc1cccc(-c2nc3cnccc3nc2N2CCN(Oc3ccc(S(C)(=O)=O)cc3F)CC2)n1] | 1-[(2-fluoro-4- methanesulfonylphenyl)m ethyl]-4-[3-(6- methoxypyridin-2- yl)pyrido[3,4-b]pyrazin-2- yl]piperazine | NA |
| 30 | 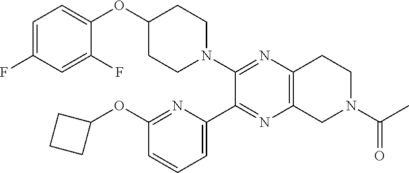 [SMILES: CC(=O)N1CCc2nc(N3CCC(Oc4ccc(F)cc4F)CC3)c(-c3cccc(OC4CCC4)n3)nc2C1] | 1-[3-(6- cyclobutoxypyridin-2-yl)- 2-[4-(2,4- difluorophenoxy)piperidin- 1-yl]-5H,6H,7H,8H- pyrido[3,4-b]pyrazin-6- yl]ethan-1-one | NA |
| 31 | 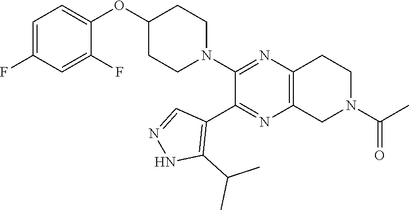 [SMILES: CC(=O)N1CCc2nc(N3CCC(Oc4ccc(F)cc4F)CC3)c(-c3cn[nH]c3C(C)C)nc2C1] | 1-{2-[4-(2,4- difluorophenoxy)piperidin- 1-yl]-3-[5-(propan-2-yl)- 1H-pyrazol-4-yl]- 5H,6H,7H,8H-pyrido[3,4- b]pyrazin-6-yl}ethan-1- one | NA |
| 32 | 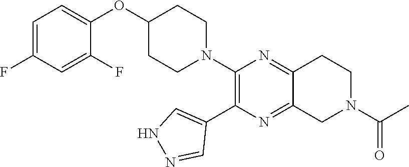 [SMILES: CC(=O)N1CCc2nc(N3CCC(Oc4ccc(F)cc4F)CC3)c(-c3cn[nH]c3)nc2C1] | 1-{2-[4-(2,4- difluorophenoxy)piperidin- 1-yl]-3-(1H-pyrazol-4-yl)- 5H,6H,7H,8H-pyrido[3,4- b]pyrazin-6-yl}ethan-1- one | NA |
| 33 | 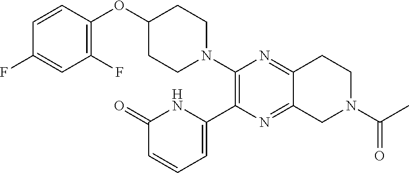 [SMILES: CC(=O)N1CCc2nc(N3CCC(Oc4ccc(F)cc4F)CC3)c(-c3cccc(=O)[nH]3)nc2C1] | 6-{6-acetyl-2-[4-(2,4- difluorophenoxy)piperidin- 1-yl]-5H,6H,7H,8H- pyrido[3,4-b]pyrazin-3- yl}-1,2-dihydropyridin-2- one | NA |
| 34 | 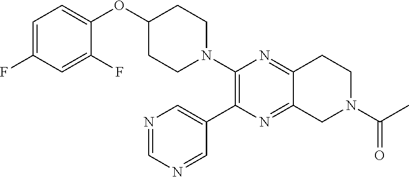 [SMILES: CC(=O)N1CCc2nc(N3CCC(Oc4ccc(F)cc4F)CC3)c(-c3cncnc3)nc2C1] | 1-{2-[4-(2,4- difluorophenoxy)piperidin- 1-yl]-3-(pyrimidin-5-yl)- 5H,6H,7H,8H-pyrido [3,4- b]pyrazin-6-yl}ethan-1- one | NA |
| 35 | 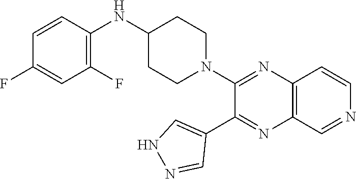 [SMILES: Fc1ccc(NC2CCN(c3nc4ccncc4nc3-c3cn[nH]c3)CC2)c(F)c1] | N-(2,4-difluorophenyl)-1- [3-(1H-pyrazol-4- yl)pyrido[3,4-b]pyrazin-2- yl]piperidin-4-amine | NA |
| 36 | 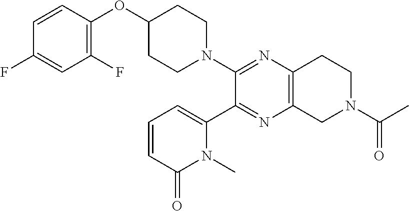 [SMILES: CC(=O)N1CCc2nc(N3CCC(Oc4ccc(F)cc4F)CC3)c(-c3cccc(=O)n3C)nc2C1] | 6-{6-acetyl-2-[4-(2,4- difluorophenoxy)piperidin- 1-yl]-5H,6H,7H,8H- pyrido[3,4-b]pyrazin-3- yl}-1-methyl-1,2- dihydropyridin-2-one | NA |
| 37 | 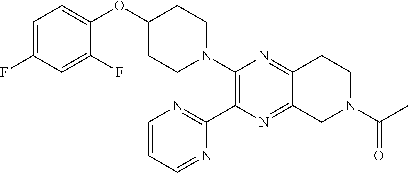 [SMILES: CC(=O)N1CCc2nc(N3CCC(Oc4ccc(F)cc4F)CC3)c(-c3ncccn3)nc2C1] | 1-{2-[4-(2,4- difluorophenoxy)piperidin- 1-yl]-3-(pyrimidin-2-yl)- 5H,6H,7H,8H-pyrido[3,4- b]pyrazin-6-yl}ethan-1- one | NA |
| 38 | 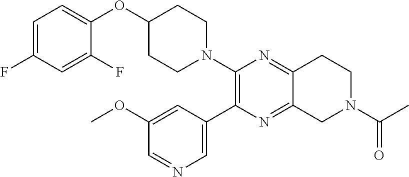 [SMILES: COc1cncc(-c2nc3c(nc2N2CCC(Oc4ccc(F)cc4F)CC2)CCN(C(C)=O)C3)c1] | 1-{2-[4-(2,4- difluorophenoxy)piperidin- 1-yl]-3-(5- methoxypyridin-3-yl)- 5H,6H,7H,8H-pyrido[3,4- b]pyrazin-6-yl}ethan-1- one | NA |
| 39 | 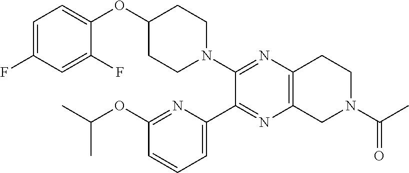 [SMILES: CC(=O)N1CCc2nc(N3CCC(Oc4ccc(F)cc4F)CC3)c(-c3cccc(OC(C)C)n3)nc2C1] | 1-{2-[4-(2,4- difluorophenoxy)piperidin- 1-yl]-3-[6-(propan-2- yloxy)pyridin-2-yl]- 5H,6H,7H,8H-pyrido[3,4- b]pyrazin-6-yl}ethan-1- one | NA |
| 40 | 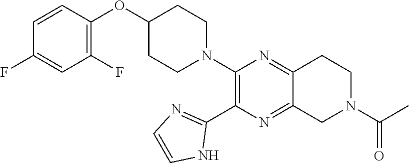 [SMILES: CC(=O)N1CCc2nc(N3CCC(Oc4ccc(F)cc4F)CC3)c(-c3ncc[nH]3)nc2C1] | 1-{2-[4-(2,4- difluorophenoxy)piperidin- 1-yl]-3-(1H-imidazol-2- yl)-5H,6H,7H,8H- pyrido[3,4-b]pyrazin-6- yl}ethan-1-one | NA |
| 41 | 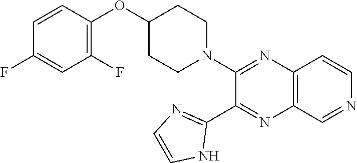 [SMILES: Fc1ccc(OC2CCN(c3nc4ccncc4nc3-c3ncc[nH]3)CC2)c(F)c1] | 4-(2,4-difluorophenoxy)- 1-[3-(1H-imidazol-2- yl)pyrido[3,4-b]pyrazin-2- yl]piperidine | NA |
| 42 | 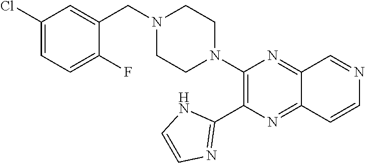 [SMILES: Fc1ccc(Cl)cc1CN1CCN(c2nc3cnccc3nc2-c2ncc[nH]2)CC1] | 1-[(5-chloro-2- fluorophenyl)methyl]-4- [2-(1H-imidazol-2- yl)pyrido[3,4-b]pyrazin-3- yl]piperazine | NA |
| 43 | 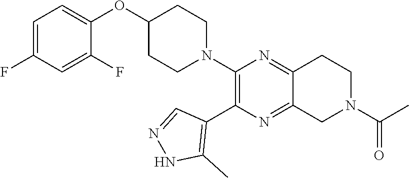 [SMILES: CC(=O)N1CCc2nc(N3CCC(Oc4ccc(F)cc4F)CC3)c(-c3cn[nH]c3C)nc2C1] | 1-{2-[4-(2,4- difluorophenoxy)piperidin- 1-yl]-3-(5-methyl-1H- pyrazol-4-yl)- 5H,6H,7H,8H-pyrido[3,4- b]pyrazin-6-yl}ethan-1- one | NA |
| 44 | 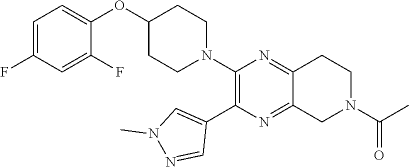 [SMILES: CC(=O)N1CCc2nc(N3CCC(Oc4ccc(F)cc4F)CC3)c(-c3cnn(C)c3)nc2C1] | 1-{2-[4-(2,4- difluorophenoxy)piperidin- 1-yl]-3-(1-methyl-1H- pyrazol-4-yl)- 5H,6H,7H,8H-pyrido[3,4- b]pyrazin-6-yl}ethan-1- one | NA |
| 45 | 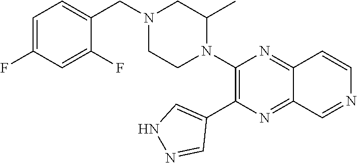 [SMILES: CC1CN(Cc2ccc(F)cc2F)CCN1c1nc2ccncc2nc1-c1cn[nH]c1] or enantiomer | 4-[(2,4- difluorophenyl)methyl]-2- methyl-1-[3-(1H-pyrazol- 4-yl)pyrido[3,4-b]pyrazin- 2-yl]piperazine, or enantiomer | NA |
| 46 | 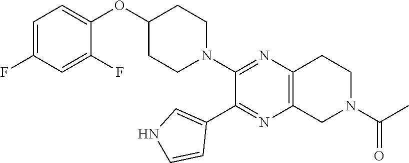 [SMILES: CC(=O)N1CCc2nc(N3CCC(Oc4ccc(F)cc4F)CC3)c(-c3cc[nH]c3)nc2C1] | 1-{2-[4-(2,4- difluorophenoxy)piperidin- 1-yl]-3-(1H-pyrrol-3-yl)- 5H,6H,7H,8H-pyrido[3,4- b]pyrazin-6-yl}ethan-1- one | NA |
| 47 | 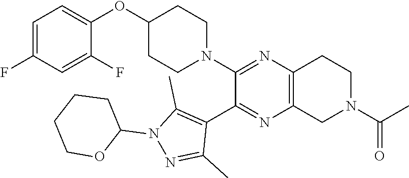 [SMILES: CC(=O)N1CCc2nc(N3CCC(Oc4ccc(F)cc4F)CC3)c(-c3c(C)nn(C4CCCCO4)c3C)nc2C1] | 1-{2-[4-(2,4- difluorophenoxy)piperidin- 1-yl]-3-[3,5-dimethyl-1- (oxan-2-yl)-1H-pyrazol-4- yl]-5H,6H,7H,8H- pyrido[3,4-b]pyrazin-6- yl}ethan-1-one | NA |
| 48 | 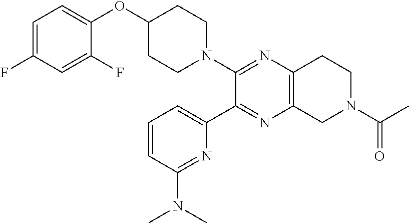 [SMILES: CC(=O)N1CCc2nc(N3CCC(Oc4ccc(F)cc4F)CC3)c(-c3cccc(N(C)C)n3)nc2C1] | 1-{2-[4-(2,4- difluorophenoxy)piperidin- 1-yl]-3-[6- (dimethylamino)pyridin- 2-yl]-5H,6H,7H,8H- pyrido[3,4-b]pyrazin-6- yl}ethan-1-one | NA |
| 49 | 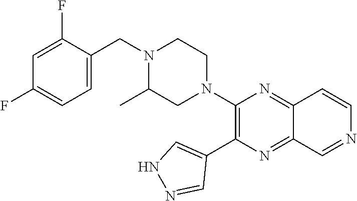 [SMILES: CC1CN(c2nc3ccncc3nc2-c2cn[nH]c2)CCN1Cc1ccc(F)cc1F] or enantiomer | 1-[(2,4- difluorophenyl)methyl]-2- methyl-4-[3-(1H-pyrazol- 4-yl)pyrido[3,4-b]pyrazin- 2-yl]piperazine, or enantiomer | NA |
| 50 | 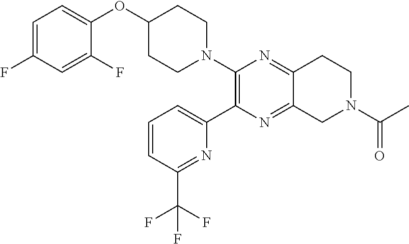 [SMILES: CC(=O)N1CCc2nc(N3CCC(Oc4ccc(F)cc4F)CC3)c(-c3cccc(C(F)(F)F)n3)nc2C1] | 1-{2-[4-(2,4- difluorophenoxy)piperidin- 1-yl]-3-[6- (trifluoromethyl)pyridin- 2-yl]-5H,6H,7H,8H- pyrido[3,4-b]pyrazin-6- yl}ethan-1-one | NA |
| 51 | 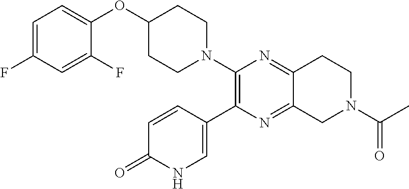 [SMILES: CC(=O)N1CCc2nc(N3CCC(Oc4ccc(F)cc4F)CC3)c(-c3ccc(=O)[nH]c3)nc2C1] | 5-{6-acetyl-2-[4-(2,4- difluorophenoxy)piperidin- 1-yl]-5H,6H,7H,8H- pyrido[3,4-b]pyrazin-3- yl}-1,2-dihydropyridin-2- one | NA |
| 52 | 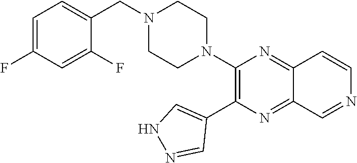 [SMILES: Fc1ccc(CN2CCN(c3nc4ccncc4nc3-c3cn[nH]c3)CC2)c(F)c1] | 1-[(2,4- difluorophenyl)methyl]-4- [3-(1H-pyrazol-4- yl)pyrido[3,4-b]pyrazin-2- yl]piperazine | NA |
| 53 | 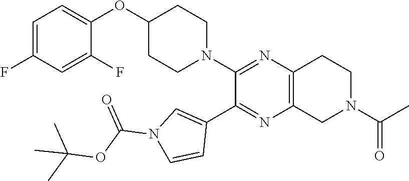 [SMILES: CC(=O)N1CCc2nc(N3CCC(Oc4ccc(F)cc4F)CC3)c(-c3ccn(C(=O)OC(C)(C)C)c3)nc2C1] | tert-butyl 3-{6-acetyl-2-[4- (2,4- difluorophenoxy)piperidin- 1-yl]-5H,6H,7H,8H- pyrido[3,4-b]pyrazin-3- yl}-1H-pyrrole-1- carboxylate | NA |
| 54 | 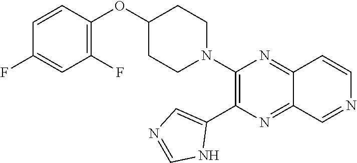 [SMILES: Fc1ccc(OC2CCN(c3nc4ccncc4nc3-c3cnc[nH]3)CC2)c(F)c1] | 4-(2,4-difluorophenoxy)- 1-[3-(1H-imidazol-5- yl)pyrido[3,4-b]pyrazin-2- yl]piperidine | NA |
| 55 | 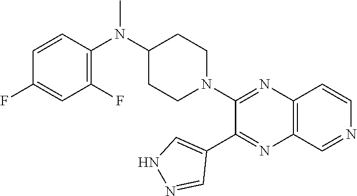 [SMILES: CN(c1ccc(F)cc1F)C1CCN(c2nc3ccncc3nc2-c2cn[nH]c2)CC1] | N-(2,4-difluorophenyl)-N- methyl-1-[3-(1H-pyrazol- 4-yl)pyrido[3,4-b]pyrazin- 2-yl]piperidin-4-amine | NA |
| 56 | 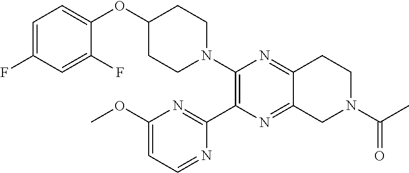 [SMILES: COc1ccnc(-c2nc3c(nc2N2CCC(Oc4ccc(F)cc4F)CC2)CCN(C(C)=O)C3)n1] | 1-{2-[4-(2,4- difluorophenoxy)piperidin- 1-yl]-3-(4- methoxypyrimidin-2-yl)- 5H,6H,7H,8H-pyrido[3,4- b]pyrazin-6-yl}ethan-1- one | NA |
| 57 | 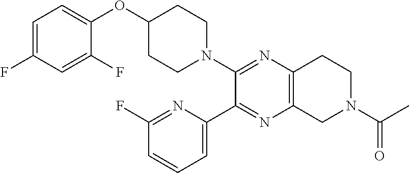 [SMILES: CC(=O)N1CCc2nc(N3CCC(Oc4ccc(F)cc4F)CC3)c(-c3cccc(F)n3)nc2C1] | 1-{2-[4-(2,4- difluorophenoxy)piperidin- 1-yl]-3-(6-fluoropyridin- 2-yl)-5H,6H,7H,8H- pyrido[3,4-b]pyrazin-6- yl}ethan-1-one | NA |
| 58 | 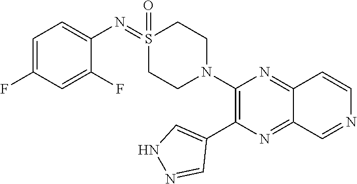 [SMILES: O=S1(=Nc2ccc(F)cc2F)CCN(c2nc3ccncc3nc2-c2cn[nH]c2)CC1] | 1-[(2,4- difluorophenyl)imino]-4- [3-(1H-pyrazol-4- yl)pyrido[3,4-b]pyrazin-2- yl]-1λ⁶-thiomorpholin-1- one | NA |
| 59 | 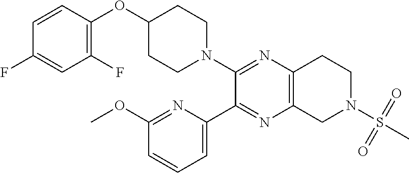 [SMILES: COc1cccc(-c2nc3c(nc2N2CCC(Oc4ccc(F)cc4F)CC2)CCN(S(C)(=O)=O)C3)n1] | 2-{2-[4-(2,4- difluorophenoxy)piperidin- 1-yl]-6-methanesulfonyl- 5H,6H,7H,8H-pyrido[3,4- b]pyrazin-3-yl}-6- methoxypyridine | NA |
| 60 | 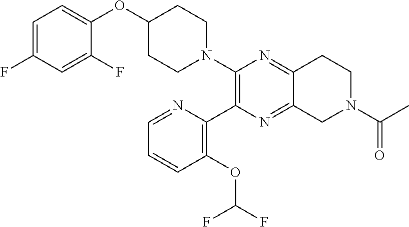 [SMILES: CC(=O)N1CCc2nc(N3CCC(Oc4ccc(F)cc4F)CC3)c(-c3ncccc3OC(F)F)nc2C1] | 1-{3-[6- (difluoromethoxy)pyridin- 2-yl]-2-[4-(2,4- difluorophenoxy)piperidin- 1-yl]-5H,6H,7H,8H- pyrido[3,4-b]pyrazin-6- yl}ethan-1-one | NA |
| 61 | 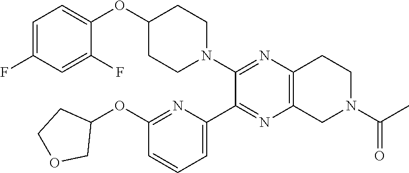 [SMILES: CC(=O)N1CCc2nc(N3CCC(Oc4ccc(F)cc4F)CC3)c(-c3cccc(OC4CCOC4)n3)nc2C1] | 1-{2-[4-(2,4- difluorophenoxy)piperidin- 1-yl]-3-[6-(oxolan-3- yloxy)pyridin-2-yl]- 5H,6H,7H,8H-pyrido[3,4- b]pyrazin-6-yl}ethan-1- one | NA |
| 62 | 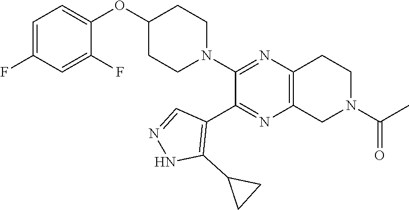 [SMILES: CC(=O)N1CCc2nc(N3CCC(Oc4ccc(F)cc4F)CC3)c(-c3cn[nH]c3C3CC3)nc2C1] | 1-[3-(5-cyclopropyl-1H- pyrazol-4-yl)-2-[4-(2,4- difluorophenoxy)piperidin- 1-yl]-5H,6H,7H,8H- pyrido[3,4-b]pyrazin-6- yl]ethan-1-one | NA |
| 63 | 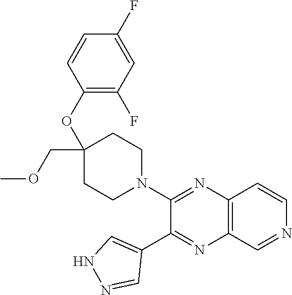 [SMILES: COCC1(Oc2ccc(F)cc2F)CCN(c2nc3ccncc3nc2-c2cn[nH]c2)CC1] | 4-(2,4-difluorophenoxy)- 4-(methoxymethyl)-1-[3- (1H-pyrazol-4- yl)pyrido[3,4-b]pyrazin-2- yl]piperidine | NA |
| 64 | 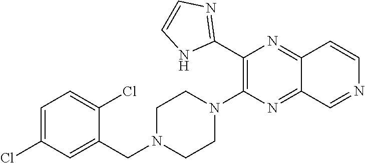 [SMILES: Clc1ccc(Cl)c(CN2CCN(c3nc4cnccc4nc3-c3ncc[nH]3)CC2)c1] | 1-[(2,5- dichlorophenyl)methyl]-4- [2-(1H-imidazol-2- yl)pyrido[3,4-b]pyrazin-3- yl]piperazine | NA |
| 65 | 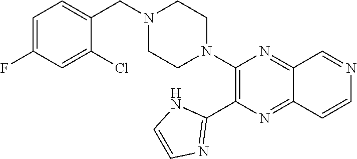 [SMILES: Fc1ccc(CN2CCN(c3nc4cnccc4nc3-c3ncc[nH]3)CC2)c(Cl)c1] | 1-[(2-chloro-4- fluorophenyl)methyl]-4- [2-(1H-imidazol-2- yl)pyrido[3,4-b]pyrazin-3- yl]piperazine | NA |
| 66 | 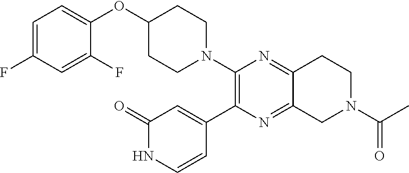 [SMILES: CC(=O)N1CCc2nc(N3CCC(Oc4ccc(F)cc4F)CC3)c(-c3cc[nH]c(=O)c3)nc2C1] | 4-{6-acetyl-2-[4-(2,4- difluorophenoxy)piperidin- 1-yl]-5H,6H,7H,8H- pyrido[3,4-b]pyrazin-3- yl}-1,2-dihydropyridin-2- one | NA |
| 67 | 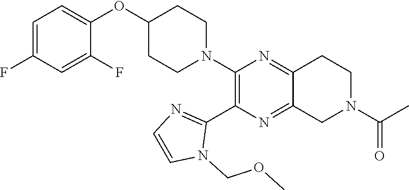 [SMILES: COCn1ccnc1-c1nc2c(nc1N1CCC(Oc3ccc(F)cc3F)CC1)CCN(C(C)=O)C2] | 1-{2-[4-(2,4- difluorophenoxy)piperidin- 1-yl]-3-[1- (methoxymethyl)-1H- imidazol-2-yl]- 5H,6H,7H,8H-pyrido[3,4- b]pyrazin-6-yl}ethan-1- one | NA |
| 68 | 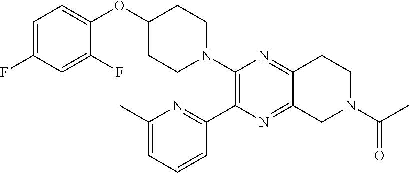 [SMILES: CC(=O)N1CCc2nc(N3CCC(Oc4ccc(F)cc4F)CC3)c(-c3cccc(C)n3)nc2C1] | 1-{2-[4-(2,4- difluorophenoxy)piperidin- 1-yl]-3-(6-methylpyridin- 2-yl)-5H,6H,7H,8H- pyrido[3,4-b]pyrazin-6- yl}ethan-1-one | NA |
| 69 | 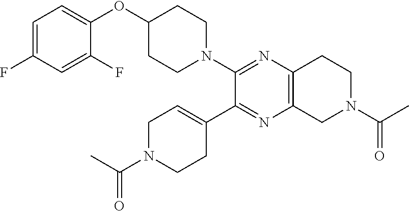 [SMILES: CC(=O)N1CC=C(c2nc3c(nc2N2CCC(Oc4ccc(F)cc4F)CC2)CCN(C(C)=O)C3)CC1] | 1-[3-(1-acetyl-1,2,3,6- tetrahydropyridin-4-yl)-2- [4-(2,4- difluorophenoxy)piperidin- 1-yl]-5H,6H,7H,8H- pyrido[3,4-b]pyrazin-6- yl]ethan-1-one | NA |
| 70 | 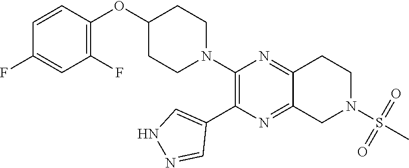 [SMILES: CS(=O)(=O)N1CCc2nc(N3CCC(Oc4ccc(F)cc4F)CC3)c(-c3cn[nH]c3)nc2C1] | 4-(2,4-difluorophenoxy)- 1-[6-methanesulfonyl-3- (1H-pyrazol-4-yl)- 5H,6H,7H,8H-pyrido[3,4- b]pyrazin-2-yl]piperidine | NA |
| 71 | 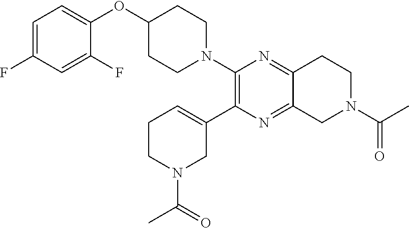 [SMILES: CC(=O)N1CCC=C(c2nc3c(nc2N2CCC(Oc4ccc(F)cc4F)CC2)CCN(C(C)=O)C3)C1] | 1-[3-(1-acetyl-1,2,5,6- tetrahydropyridin-3-yl)-2- [4-(2,4- difluorophenoxy)piperidin- 1-yl]-5H,6H,7H,8H- pyrido[3,4-b]pyrazin-6- yl]ethan-1-one | NA |
| 72 | 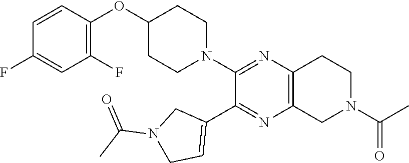 [SMILES: CC(=O)N1CC=C(c2nc3c(nc2N2CCC(Oc4ccc(F)cc4F)CC2)CCN(C(C)=O)C3)C1] | 1-(3-{6-acetyl-2-[4-(2,4- difluorophenoxy)piperidin- 1-yl]-5H,6H,7H,8H- pyrido[3,4-b]pyrazin-3- yl}-2,5-dihydro-1H-pyrrol- 1-yl)ethan-1-one | NA |
| 73 | 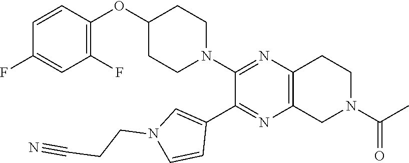 [SMILES: CC(=O)N1CCc2nc(N3CCC(Oc4ccc(F)cc4F)CC3)c(-c3ccn(CCC#N)c3)nc2C1] | 3-(4-{6-acetyl-2-[4-(2,4- difluorophenoxy)piperidin- 1-yl]-5H,6H,7H,8H- pyrido[3,4-b]pyrazin-3- yl}-1H-pyrazol-1- yl)propanenitrile | NA |
| 74 | 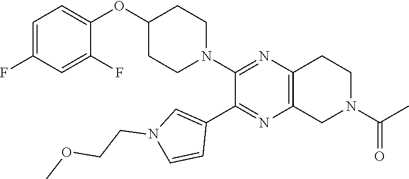 [SMILES: COCCn1ccc(-c2nc3c(nc2N2CCC(Oc4ccc(F)cc4F)CC2)CCN(C(C)=O)C3)c1] | 1-{2-[4-(2,4- difluorophenoxy)piperidin- 1-yl]-3-[1-(2- methoxyethyl)-1H- pyrazol-4-yl]- 5H,6H,7H,8H-pyrido[3,4- b]pyrazin-6-yl}ethan-1- one | NA |
| 75 | 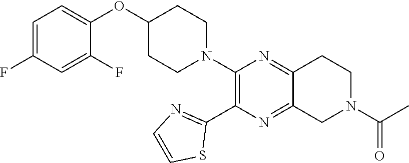 [SMILES: CC(=O)N1CCc2nc(N3CCC(Oc4ccc(F)cc4F)CC3)c(-c3nccs3)nc2C1] | 1-{2-[4-(2,4- difluorophenoxy)piperidin- 1-yl]-3-(1,3-thiazol-2-yl)- 5H,6H,7H,8H-pyrido[3,4- b]pyrazin-6-yl}ethan-1- one | NA |
| 76 | 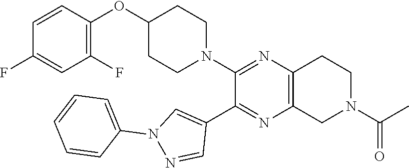 [SMILES: CC(=O)N1CCc2nc(N3CCC(Oc4ccc(F)cc4F)CC3)c(-c3cnn(-c4ccccc4)c3)nc2C1] | 1-{2-[4-(2,4- difluorophenoxy)piperidin- 1-yl]-3-(1-phenyl-1H- pyrazol-4-yl)- 5H,6H,7H,8H-pyrido[3,4- b]pyrazin-6-yl}ethan-1- one | NA |
| 77 | 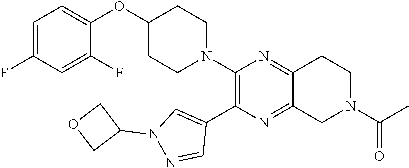 [SMILES: CC(=O)N1CCc2nc(N3CCC(Oc4ccc(F)cc4F)CC3)c(-c3cnn(C4COC4)c3)nc2C1] | 1-{2-[4-(2,4- difluorophenoxy)piperidin- 1-yl]-3-[1-(oxetan-3-yl)- 1H-pyrazol-4-yl]- 5H,6H,7H,8H-pyrido [3,4- b]pyrazin-6-yl}ethan-1- one | NA |
| 78 | 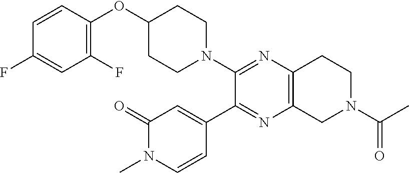 [SMILES: CC(=O)N1CCc2nc(N3CCC(Oc4ccc(F)cc4F)CC3)c(-c3ccn(C)c(=O)c3)nc2C1] | 4-{6-acetyl-2-[4-(2,4- difluorophenoxy)piperidin- 1-yl]-5H,6H,7H,8H- pyrido[3,4-b]pyrazin-3- yl}-1-methyl-1,2- dihydropyridin-2-one | NA |
| 79 | 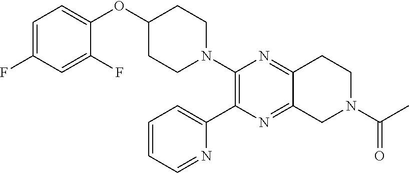 [SMILES: CC(=O)N1CCc2nc(N3CCC(Oc4ccc(F)cc4F)CC3)c(-c3ccccn3)nc2C1] | 1-{2-[4-(2,4- difluorophenoxy)piperidin- 1-yl]-3-(pyridin-2-yl)- 5H,6H,7H,8H-pyrido[3,4- b]pyrazin-6-yl}ethan-1- one | NA |
| 80 | 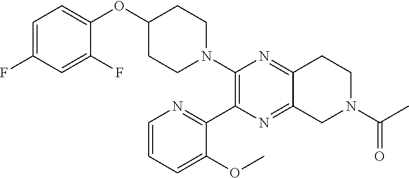 [SMILES: COc1cccnc1-c1nc2c(nc1N1CCC(Oc3ccc(F)cc3F)CC1)CCN(C(C)=O)C2] | 1-{2-[4-(2,4- difluorophenoxy)piperidin- 1-yl]-3-(3- methoxypyridin-2-yl)- 5H,6H,7H,8H-pyrido[3,4- b]pyrazin-6-yl}ethan-1- one | NA |
| 81 | 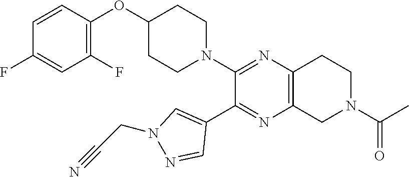 [SMILES: CC(=O)N1CCc2nc(N3CCC(Oc4ccc(F)cc4F)CC3)c(-c3cnn(CC#N)c3)nc2C1] | 2-(4-{6-acetyl-2-[4-(2,4- difluorophenoxy)piperidin- 1-yl]-5H,6H,7H,8H- pyrido[3,4-b]pyrazin-3- yl}-1H-pyrazol-1- yl)acetonitrile | NA |
| 82 | 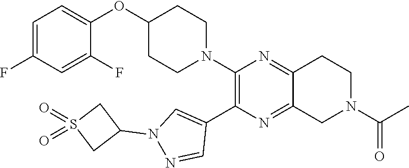 [SMILES: CC(=O)N1CCc2nc(N3CCC(Oc4ccc(F)cc4F)CC3)c(-c3cnn(C4CS(=O)(=O)C4)c3)nc2C1] | 3-(4-{6-acetyl-2-[4-(2,4- difluorophenoxy)piperidin- 1-yl]-5H,6H,7H,8H- pyrido[3,4-b]pyrazin-3- yl}-1H-pyrazol-1-yl)-1λ⁶- thietane-1,1-dione | NA |
| 83 | 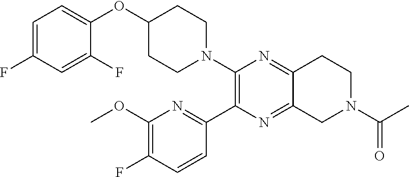 [SMILES: COc1nc(-c2nc3c(nc2N2CCC(Oc4ccc(F)cc4F)CC2)CCN(C(C)=O)C3)ccc1F] | 1-{2-[4-(2,4- difluorophenoxy)piperidin- 1-yl]-3-(5-fluoro-6- methoxypyridin-2-yl)- 5H,6H,7H,8H-pyrido[3,4- b]pyrazin-6-yl}ethan-1- one | NA |
| 84 | 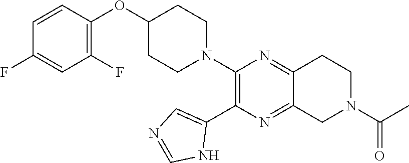 [SMILES: CC(=O)N1CCc2nc(N3CCC(Oc4ccc(F)cc4F)CC3)c(-c3cnc[nH]3)nc2C1] | 1-{2-[4-(2,4- difluorophenoxy)piperidin- 1-yl]-3-(1H-imidazol-5- yl)-5H,6H,7H,8H- pyrido[3,4-b]pyrazin-6- yl}ethan-1-one | NA |
| 85 | 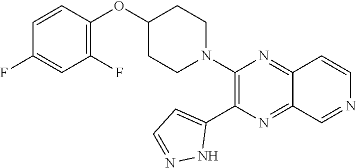 [SMILES: Fc1ccc(OC2CCN(c3nc4ccncc4nc3-c3ccn[nH]3)CC2)c(F)c1] | 4-(2,4-difluorophenoxy)- 1-[3-(1H-pyrazol-5- yl)pyrido[3,4-b]pyrazin-2- yl]piperidine | NA |
| 86 | 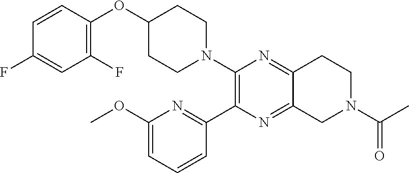 [SMILES: COc1cccc(-c2nc3c(nc2N2CCC(Oc4ccc(F)cc4F)CC2)CCN(C(C)=O)C3)n1] | 1-{2-[4-(2,4- difluorophenoxy)piperidin- 1-yl]-3-(6- methoxypyridin-2-yl)- 5H,6H,7H,8H-pyrido[3,4- b]pyrazin-6-yl}ethan-1- one | NA |
| 87 | 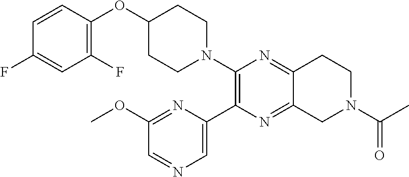 [SMILES: COc1cncc(-c2nc3c(nc2N2CCC(Oc4ccc(F)cc4F)CC2)CCN(C(C)=O)C3)n1] | 1-{2-[4-(2,4- difluorophenoxy)piperidin- 1-yl]-3-(6- methoxypyrazin-2-yl)- 5H,6H,7H,8H-pyrido[3,4- b]pyrazin-6-yl}ethan-1- one | NA |
| 88 | 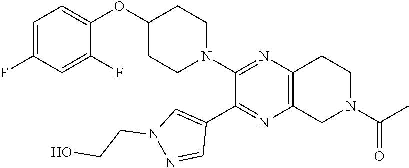 [SMILES: CC(=O)N1CCc2nc(N3CCC(Oc4ccc(F)cc4F)CC3)c(-c3cnn(CCO)c3)nc2C1] | 1-{2-[4-(2,4- difluorophenoxy)piperidin- 1-yl]-3-[1-(2- hydroxyethyl)-1H- pyrazol-4-yl]- 5H,6H,7H,8H-pyrido[3,4- b]pyrazin-6-yl}ethan-1- one | NA |
| 89 | 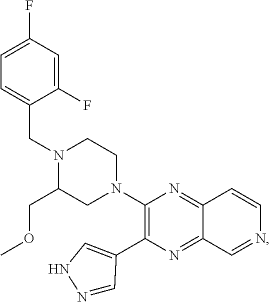 [SMILES: COCC1CN(c2nc3ccncc3nc2-c2cn[nH]c2)CCN1Cc1ccc(F)cc1F] or enantiomer | 1-[(2,4- difluorophenyl)methyl]-2- (methoxymethyl)-4-[3- (1H-pyrazol-4- yl)pyrido[3,4-b]pyrazin-2- yl]piperazine, or enantiomer | NA |
| 90 | 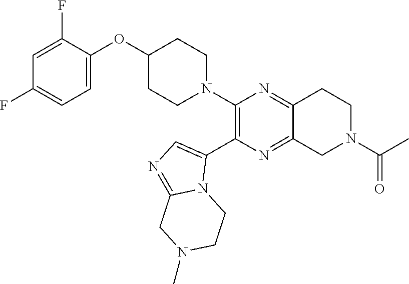 [SMILES: CC(=O)N1CCc2nc(N3CCC(Oc4ccc(F)cc4F)CC3)c(-c3cnc4n3CCN(C)C4)nc2C1] | 1-(2-(4-(2,4- difluorophenoxy)piperidin- 1-yl)-3-(7-methyl-5,6,7,8- tetrahydroimidazo[1,2- a]pyrazin-3-yl)-7,8- dihydropyrido[3,4- b]pyrazin-6(5H)-yl)ethan- 1-one | NA |
| 91 | 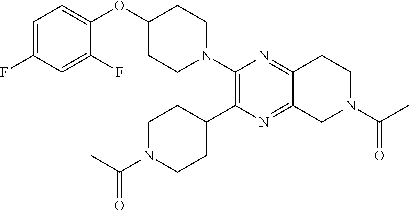 [SMILES: CC(=O)N1CCC(c2nc3c(nc2N2CCC(Oc4ccc(F)cc4F)CC2)CCN(C(C)=O)C3)CC1] | 1-(4-(6-acetyl-2-(4-(2,4- difluorophenoxy)piperidin- 1-yl)-5,6,7,8- tetrahydropyrido [3,4- b]pyrazin-3-yl)piperidin- 1-yl)ethan-1-one | NA |
| 92 | 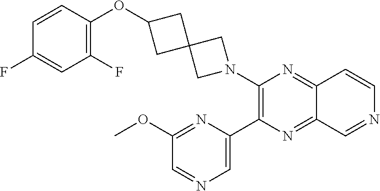 [SMILES: COc1cncc(-c2nc3cnccc3nc2N2CC3(CC(Oc4ccc(F)cc4F)C3)C2)n1] | 2-(6-(2,4- difluorophenoxy)-2- azaspiro[3.3]heptan-2-yl)- 3-(6-methoxypyrazin-2- yl)pyrido[3,4-b]pyrazine | NA |
| 93 | 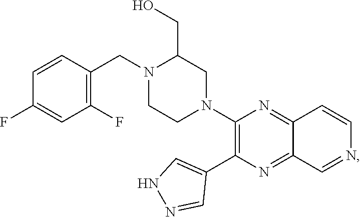 [SMILES: OCC1CN(c2nc3ccncc3nc2-c2cn[nH]c2)CCN1Cc1ccc(F)cc1F] or enantiomer | (4-(3-(1H-pyrazol-4- yl)pyrido[3,4-b]pyrazin-2- yl)-1-(2,4- difluorobenzyl)piperazin- 2-yl)methanol, or enantiomer | NA |
| 94 | 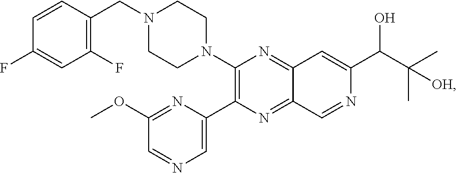 [SMILES: COc1cncc(-c2nc3cnc(C(O)C(C)(C)O)cc3nc2N2CCN(Cc3ccc(F)cc3F)CC2)n1] or enantiomer | 1-(2-(4-(2,4- difluorobenzyl)piperazin- 1-yl)-3-(6- methoxypyrazin-2- yl)pyrido[3,4-b]pyrazin-7- yl)-2-methylpropane-1,2- diol, or enantiomer | NA |
| 95 | 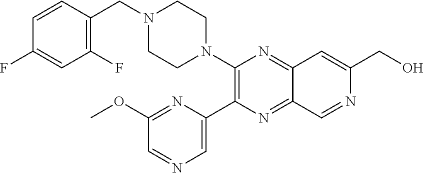 [SMILES: COc1cncc(-c2nc3cnc(CO)cc3nc2N2CCN(Cc3ccc(F)cc3F)CC2)n1] | (2-(4-(2,4- difluorobenzyl)piperazin- 1-yl)-3-(6- methoxypyrazin-2- yl)pyrido[3,4-b]pyrazin-7- yl)methanol | NA |
| 96 | 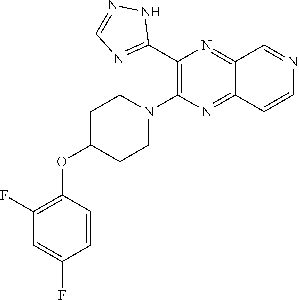 [SMILES: Fc1ccc(OC2CCN(c3nc4ccncc4nc3-c3ncn[nH]3)CC2)c(F)c1] | 4-(2,4-difluorophenoxy)- 1-[3-(1H-1,2,4-triazol-5- yl)pyrido[3,4-b]pyrazin-2- yl]piperidine | NA |
| 97 | 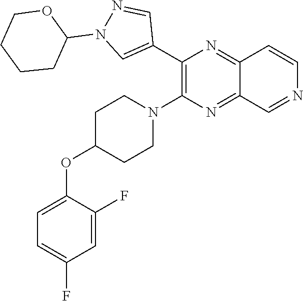 [SMILES: Fc1ccc(OC2CCN(c3nc4cnccc4nc3-c3cnn(C4CCCCO4)c3)CC2)c(F)c1] | 4-(2,4-difluorophenoxy)- 1-{2-[1-(oxan-2-yl)-1H- pyrazol-4-yl]pyrido[3,4- b]pyrazin-3-yl}piperidine | NA |
| 98 | 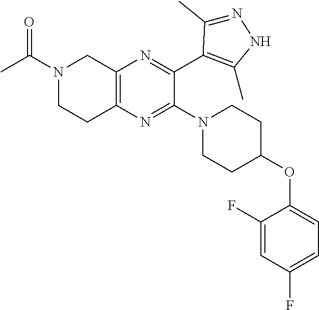 [SMILES: CC(=O)N1CCc2nc(N3CCC(Oc4ccc(F)cc4F)CC3)c(-c3c(C)n[nH]c3C)nc2C1] | 1-{2-[4-(2,4- difluorophenoxy)piperidin- 1-yl]-3-(3,5-dimethyl-1H- pyrazol-4-yl)- 5H,6H,7H,8H-pyrido[3,4- b]pyrazin-6-yl}ethan-1- one | NA |
| 99 | 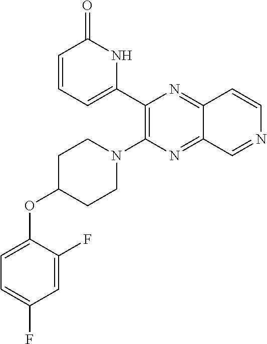 [SMILES: O=c1cccc(-c2nc3ccncc3nc2N2CCC(Oc3ccc(F)cc3F)CC2)[nH]1] | 6-{3-[4-(2,4- difluorophenoxy)piperidin- 1-yl]pyrido[3,4- b]pyrazin-2-yl}-1,2- dihydropyridin-2-one | NA |
| 100 | 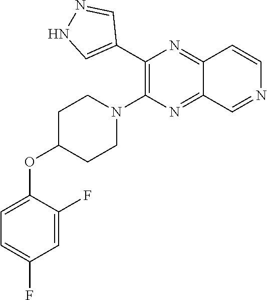 [SMILES: Fc1ccc(OC2CCN(c3nc4cnccc4nc3-c3cn[nH]c3)CC2)c(F)c1] | 4-(2,4-difluorophenoxy)- 1-[2-(1H-pyrazol-4- yl)pyrido[3,4-b]pyrazin-3- yl]piperidine | NA |
| 101 | 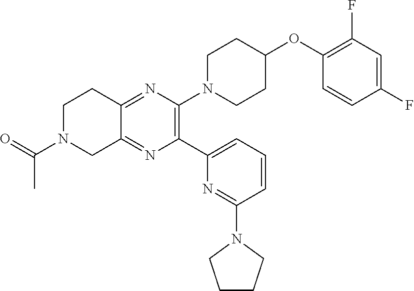 [SMILES: CC(=O)N1CCc2nc(N3CCC(Oc4ccc(F)cc4F)CC3)c(-c3cccc(N4CCCC4)n3)nc2C1] | 1-{2-[4-(2,4- difluorophenoxy)piperidin- 1-yl]-3-[6-(pyrrolidin-1- yl)pyridin-2-yl]- 5H,6H,7H,8H-pyrido[3,4- b]pyrazin-6-yl}ethan-1- one | NA |
| 102 | 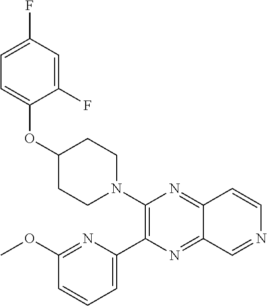 [SMILES: COc1cccc(-c2nc3cnccc3nc2N2CCC(Oc3ccc(F)cc3F)CC2)n1] | 2-{2-[4-(2,4- difluorophenoxy)piperidin- 1-yl]pyrido[3,4- b]pyrazin-3-yl}-6- methoxypyridine | NA |
| 103 | 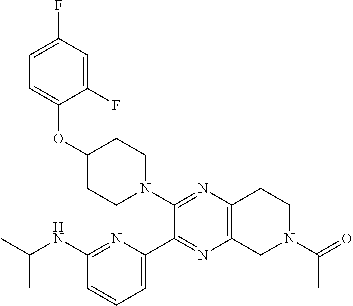 [SMILES: CC(=O)N1CCc2nc(N3CCC(Oc4ccc(F)cc4F)CC3)c(-c3cccc(NC(C)C)n3)nc2C1] | 1-{2-[4-(2,4- difluorophenoxy)piperidin- 1-yl]-3-{6-[(propan-2- yl)amino]pyridin-2-yl}- 5H,6H,7H,8H-pyrido [3,4- b]pyrazin-6-yl}ethan-1- one | NA |
| 104 | 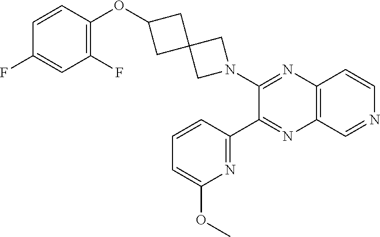 [SMILES: COc1cccc(-c2nc3cnccc3nc2N2CC3(CC(Oc4ccc(F)cc4F)C3)C2)n1] | 6-(2,4-difluorophenoxy)- 2-[3-(6-methoxypyridin-2- yl)pyrido[3,4-b]pyrazin-2- yl]-2-azaspiro[3.3]heptane | NA |
| 105 | 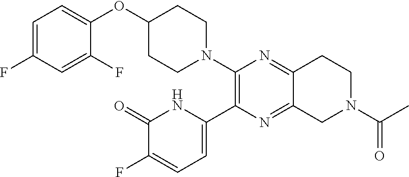 [SMILES: CC(=O)N1CCc2nc(N3CCC(Oc4ccc(F)cc4F)CC3)c(-c3ccc(F)c(=O)[nH]3)nc2C1] | 6-{6-acetyl-2-[4-(2,4- difluorophenoxy)piperidin- 1-yl]-5H,6H,7H,8H- pyrido[3,4-b]pyrazin-3- yl}-3-fluoro-1,2- dihydropyridin-2-one | NA |
| 106 | 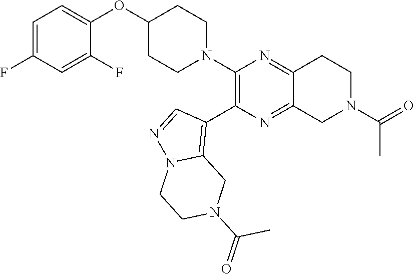 [SMILES: CC(=O)N1CCc2nc(N3CCC(Oc4ccc(F)cc4F)CC3)c(-c3cnn4c3CN(C(C)=O)CC4)nc2C1] | 1-(3-{6-acetyl-2-[4-(2,4- difluorophenoxy)piperidin- 1-yl]-5H,6H,7H,8H- pyrido[3,4-b]pyrazin-3- yl}-4H,5H,6H,7H- pyrazolo[1,5-a]pyrazin-5- yl)ethan-1-one | NA |
| 107 | 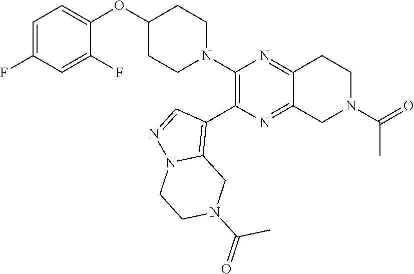 [SMILES: CC(=O)N1CCc2nc(N3CCC(Oc4ccc(F)cc4F)CC3)c(-c3cnn4c3CN(C(C)=O)CC4)nc2C1] | 1-(3-{6-acetyl-2-[4-(2,4- difluorophenoxy)piperidin- 1-yl]-5H,6H,7H,8H- pyrido[3,4-b]pyrazin-3- yl}-4H,5H,6H,7H- pyrazolo[1,5-a]pyrazin-5- yl)ethan-1-one | NA |
| 108 | 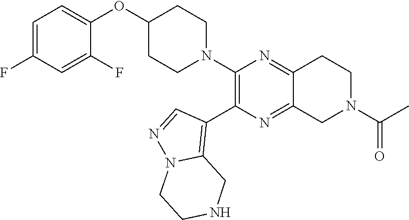 [SMILES: CC(=O)N1CCc2nc(N3CCC(Oc4ccc(F)cc4F)CC3)c(-c3cnn4c3CNCC4)nc2C1] | 1-{2-[4-(2,4- difluorophenoxy)piperidin- 1-yl]-3-{4H,5H,6H,7H- pyrazolo[1,5-a]pyrazin-3- yl}-5H,6H,7H,8H- pyrido[3,4-b]pyrazin-6- yl}ethan-1-one | NA |
| 109 | 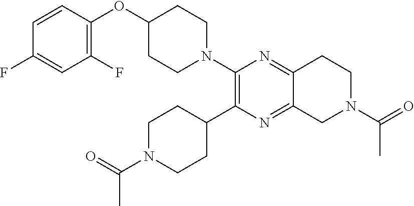 [SMILES: CC(=O)N1CCC(c2nc3c(nc2N2CCC(Oc4ccc(F)cc4F)CC2)CCN(C(C)=O)C3)CC1] | 1-[3-(1-acetylpiperidin-4- yl)-2-[4-(2,4- difluorophenoxy)piperidin- 1-yl]-5H,6H,7H,8H- pyrido[3,4-b]pyrazin-6- yl]ethan-1-one | NA |
| 110 | 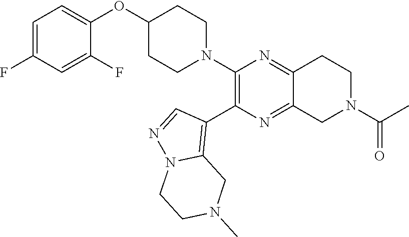 [SMILES: CC(=O)N1CCc2nc(N3CCC(Oc4ccc(F)cc4F)CC3)c(-c3cnn4c3CN(C)CC4)nc2C1] | 1-{2-[4-(2,4- difluorophenoxy)piperidin- 1-yl]-3-{5-methyl- 4H,5H,6H,7H- pyrazolo[1,5-a]pyrazin-3- yl}-5H,6H,7H,8H- pyrido[3,4-b]pyrazin-6- yl}ethan-1-one | NA |
| 111 | 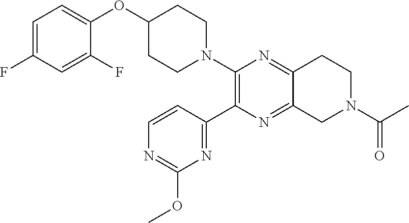 [SMILES: COc1nccc(-c2nc3c(nc2N2CCC(Oc4ccc(F)cc4F)CC2)CCN(C(C)=O)C3)n1] | 1-{2-[4-(2,4- difluorophenoxy)piperidin- 1-yl]-3-(2- methoxypyrimidin-4-yl)- 5H,6H,7H,8H-pyrido[3,4- b]pyrazin-6-yl}ethan-1- one | NA |
| 112 | 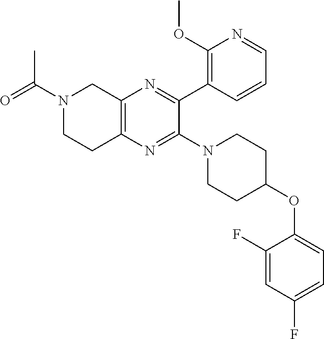 [SMILES: COc1ncccc1-c1nc2c(nc1N1CCC(Oc3ccc(F)cc3F)CC1)CCN(C(C)=O)C2] | 1-{2-[4-(2,4- difluorophenoxy)piperidin- 1-yl]-3-(2- methoxypyridin-3-yl)- 5H,6H,7H,8H-pyrido[3,4- b]pyrazin-6-yl}ethan-1- one | NA |
| 113 | 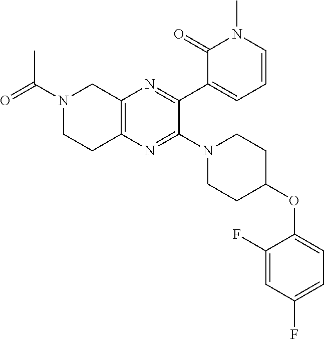 [SMILES: CC(=O)N1CCc2nc(N3CCC(Oc4ccc(F)cc4F)CC3)c(-c3cccn(C)c3=O)nc2C1] | 3-{6-acetyl-2-[4-(2,4- difluorophenoxy)piperidin- 1-yl]-5H,6H,7H,8H- pyrido[3,4-b]pyrazin-3- yl}-1-methyl-1,2- dihydropyridin-2-one | NA |
| 114 | 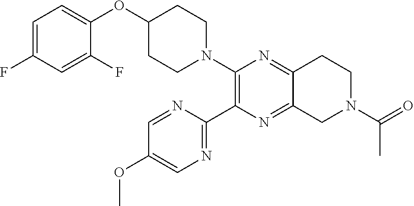 [SMILES: COc1cnc(-c2nc3c(nc2N2CCC(Oc4ccc(F)cc4F)CC2)CCN(C(C)=O)C3)nc1] | 1-{2-[4-(2,4- difluorophenoxy)piperidin- 1-yl]-3-(5- methoxypyrazin-2-yl)- 5H,6H,7H,8H-pyrido[3,4- b]pyrazin-6-yl}ethan-1- one | NA |
| 115 | 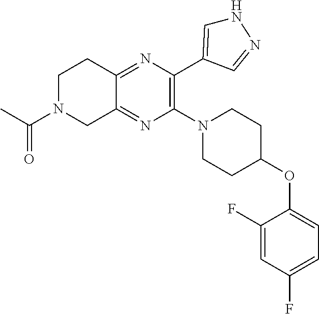 [SMILES: CC(=O)N1CCc2nc(-c3cn[nH]c3)c(N3CCC(Oc4ccc(F)cc4F)CC3)nc2C1] | 1-{3-[4-(2,4- difluorophenoxy)piperidin- 1-yl]-2-(1H-pyrazol-4-yl)- 5H,6H,7H,8H-pyrido[3,4- b]pyrazin-6-yl}ethan-1- one | NA |
| 116 | 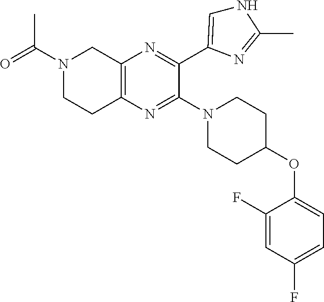 [SMILES: CC(=O)N1CCc2nc(N3CCC(Oc4ccc(F)cc4F)CC3)c(-c3c[nH]c(C)n3)nc2C1] | 1-{2-[4-(2,4- difluorophenoxy)piperidin- 1-yl]-3-(2-methyl-1H- imidazol-4-yl)- 5H,6H,7H,8H-pyrido[3,4- b]pyrazin-6-yl}ethan-1- one | NA |
| 117 | 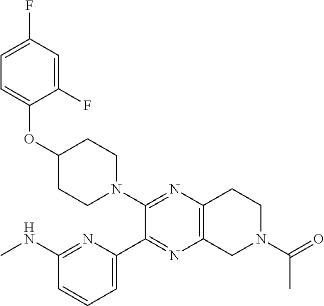 [SMILES: CNc1cccc(-c2nc3c(nc2N2CCC(Oc4ccc(F)cc4F)CC2)CCN(C(C)=O)C3)n1] | 1-{2-[4-(2,4- difluorophenoxy)piperidin- 1-yl]-3-[6- (methylamino)pyridin-2- yl]-5H,6H,7H,8H- pyrido[3,4-b]pyrazin-6- yl}ethan-1-one | NA |
| 118 | 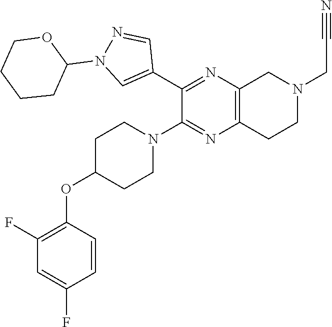 [SMILES: N#CCN1CCc2nc(N3CCC(Oc4ccc(F)cc4F)CC3)c(-c3cnn(C4CCCCO4)c3)nc2C1] | 2-{2-[4-(2,4- difluorophenoxy)piperidin- 1-yl]-3-[1-(oxan-2-yl)-1H- pyrazol-4-yl]- 5H,6H,7H,8H-pyrido[3,4- b]pyrazin-6-yl}acetonitrile | NA |
| 119 | 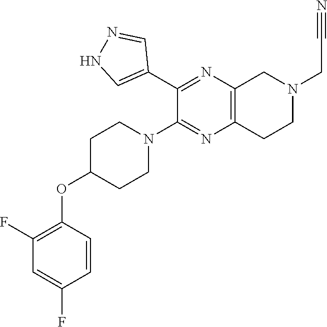 [SMILES: N#CCN1CCc2nc(N3CCC(Oc4ccc(F)cc4F)CC3)c(-c3cn[nH]c3)nc2C1] | 2-{2-[4-(2,4- difluorophenoxy)piperidin- 1-yl]-3-(1H-pyrazol-4-yl)- 5H,6H,7H,8H-pyrido[3,4- b]pyrazin-6-yl}acetonitrile | NA |
| 120 | 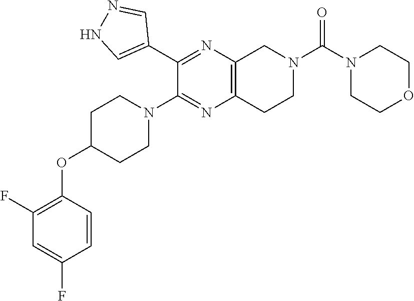 [SMILES: O=C(N1CCOCC1)N1CCc2nc(N3CCC(Oc4ccc(F)cc4F)CC3)c(-c3cn[nH]c3)nc2C1] | 4-{2-[4-(2,4- difluorophenoxy)piperidin- 1-yl]-3-(1H-pyrazol-4-yl)- 5H,6H,7H,8H-pyrido[3,4- b]pyrazine-6- carbonyl}morpholine | NA |
| 121 | 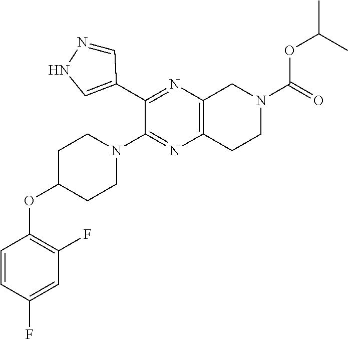 [SMILES: CC(C)OC(=O)N1CCc2nc(N3CCC(Oc4ccc(F)cc4F)CC3)c(-c3cn[nH]c3)nc2C1] | propan-2-yl 2-[4-(2,4- difluorophenoxy)piperidin- 1-yl]-3-(1H-pyrazol-4-yl)- c5H,6H,7H,8H-pyrido[3,4- b]pyrazine-6-carboxylate | NA |
| 122 | 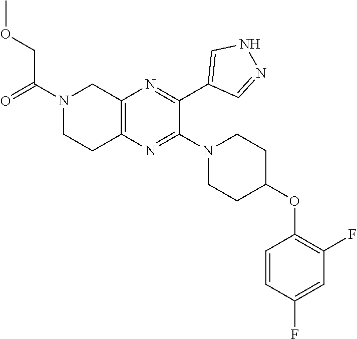 [SMILES: COCC(=O)N1CCc2nc(N3CCC(Oc4ccc(F)cc4F)CC3)c(-c3cn[nH]c3)nc2C1] | ethyl 2-[4-(2,4- difluorophenoxy)piperidin- 1-yl]-3-(1H-pyrazol-4-yl)- 5H,6H,7H,8H-pyrido[3,4- b]pyrazine-6-carboxylate | NA |
| 123 | 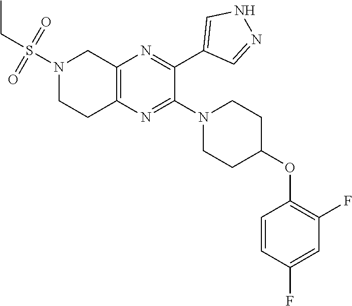 [SMILES: CCS(=O)(=O)N1CCc2nc(N3CCC(Oc4ccc(F)cc4F)CC3)c(-c3cn[nH]c3)nc2C1] | 4-(2,4-difluorophenoxy)- 1-[6-(ethanesulfonyl)-3- (1H-pyrazol-4-yl)- 5H,6H,7H,8H-pyrido[3,4- b]pyrazin-2-yl]piperidine | NA |
| 124 | 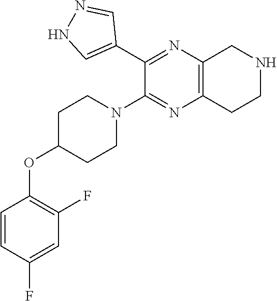 [SMILES: Fc1ccc(OC2CCN(c3nc4c(nc3-c3cn[nH]c3)CNCC4)CC2)c(F)c1] | 4-(2,4-difluorophenoxy)- 1-[3-(1H-pyrazol-4-yl)- 5H,6H,7H,8H-pyrido[3,4- b]pyrazin-2-yl]piperidine | NA |
| 125 | 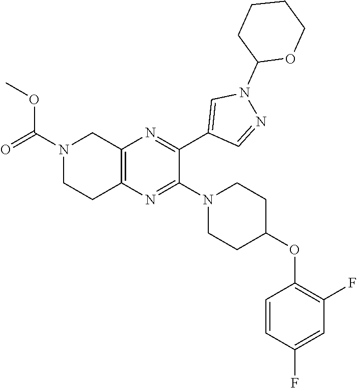 [SMILES: COC(=O)N1CCc2nc(N3CCC(Oc4ccc(F)cc4F)CC3)c(-c3cnn(C4CCCCO4)c3)nc2C1] | methyl 2-[4-(2,4- difluorophenoxy)piperidin- 1-yl]-3-[1-(oxan-2-yl)-1H- pyrazol-4-yl]- 5H,6H,7H,8H-pyrido[3,4- b]pyrazine-6-carboxylate | NA |
| 126 | 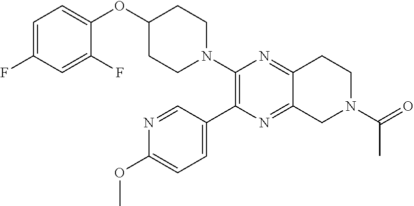 [SMILES: COc1ccc(-c2nc3c(nc2N2CCC(Oc4ccc(F)cc4F)CC2)CCN(C(C)=O)C3)cn1] | 1-{2-[4-(2,4- difluorophenoxy)piperidin- 1-yl]-3-(6- methoxypyridin-3-yl)- 5H,6H,7H,8H-pyrido[3,4- b]pyrazin-6-yl}ethan-1- one | NA |
| 127 | 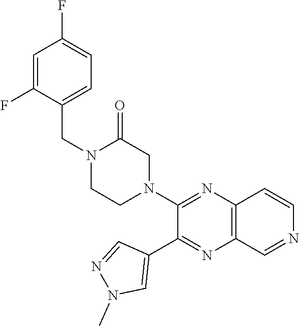 [SMILES: Cn1cc(-c2nc3cnccc3nc2N2CCN(Cc3ccc(F)cc3F)C(=O)C2)cn1] | 1-[(2,4- difluorophenyl)methyl]-4- [3-(1-methyl-1H-pyrazol- 4-yl)pyrido[3,4-b]pyrazin- 2-yl]piperazin-2-one | NA |
| 128 | 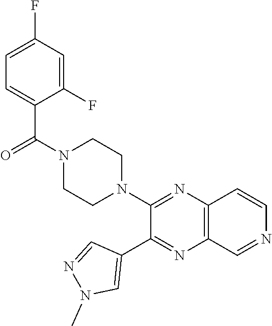 [SMILES: Cn1cc(-c2nc3cnccc3nc2N2CCN(C(=O)c3ccc(F)cc3F)CC2)cn1] | 1-(2,4-difluorobenzoyl)-4- [3-(1-methyl-1H-pyrazol- 4-yl)pyrido[3,4-b]pyrazin- 2-yl]piperazine | NA |
| 129 | 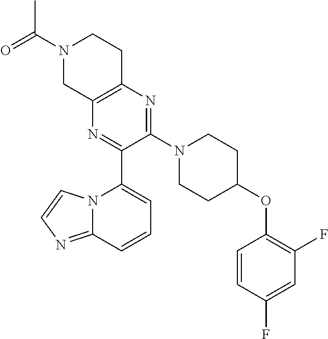 [SMILES: CC(=O)N1CCc2nc(N3CCC(Oc4ccc(F)cc4F)CC3)c(-c3cccc4nccn34)nc2C1] | 1-{2-[4-(2,4- difluorophenoxy)piperidin- 1-yl]-3-{imidazo[1,2- a]pyridin-5-yl}- 5H,6H,7H,8H-pyrido[3,4- b]pyrazin-6-yl}ethan-1- one | NA |
| 130 | 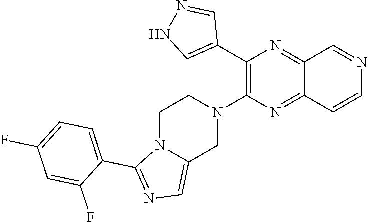 [SMILES: Fc1ccc(-c2ncc3n2CCN(c2nc4ccncc4nc2-c2cn[nH]c2)C3)c(F)c1] | 4-{2-[3-(2,4- difluorophenyl)- 5H,6H,7H,8H-imidazo[1,5- a]pyrazin-7-yl]pyrido[3,4- b]pyrazin-3-yl}-1H- pyrazole | NA |
| 131 | 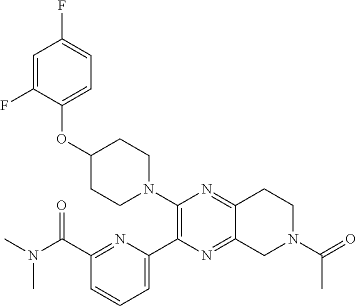 [SMILES: CC(=O)N1CCc2nc(N3CCC(Oc4ccc(F)cc4F)CC3)c(-c3cccc(C(=O)N(C)C)n3)nc2C1] | 6-{6-acetyl-2-[4-(2,4- difluorophenoxy)piperidin- 1-yl]-5H,6H,7H,8H- pyrido[3,4-b]pyrazin-3- yl}-N,N-dimethylpyridine- 2-carboxamide | NA |
| 132 | 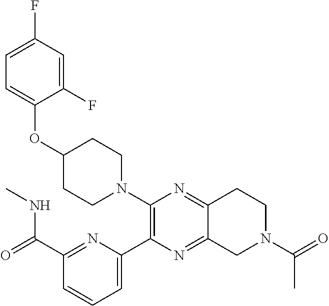 [SMILES: CNC(=O)c1cccc(-c2nc3c(nc2N2CCC(Oc4ccc(F)cc4F)CC2)CCN(C(C)=O)C3)n1] | 6-{6-acetyl-2-[4-(2,4- difluorophenoxy)piperidin- 1-yl]-5H,6H,7H,8H- pyrido[3,4-b]pyrazin-3- yl}-N-methylpyridine-2- carboxamide | NA |
| 133 | 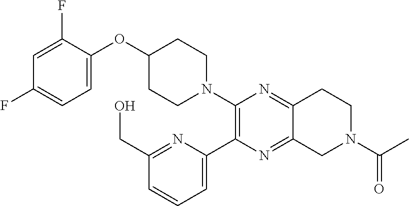 [SMILES: CC(=O)N1CCc2nc(N3CCC(Oc4ccc(F)cc4F)CC3)c(-c3cccc(CO)n3)nc2C1] | 1-{2-[4-(2,4- difluorophenoxy)piperidin- 1-yl]-3-[6- (hydroxymethyl)pyridin- 2-yl]-5H,6H,7H,8H- pyrido[3,4-b]pyrazin-6- yl}ethan-1-one | NA |
| 134 | 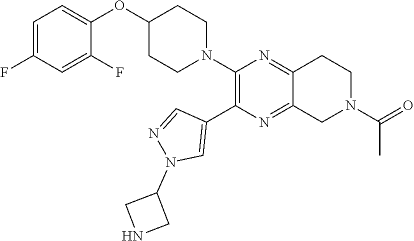 [SMILES: CC(=O)N1CCc2nc(N3CCC(Oc4ccc(F)cc4F)CC3)c(-c3cnn(C4CNC4)c3)nc2C1] | 1-{3-[1-(azetidin-3-yl)-1H- pyrazol-4-yl]-2-[4-(2,4- difluorophenoxy)piperidin- 1-yl]-5H,6H,7H,8H- pyrido[3,4-b]pyrazin-6- yl}ethan-1-one | NA |
| 135 | 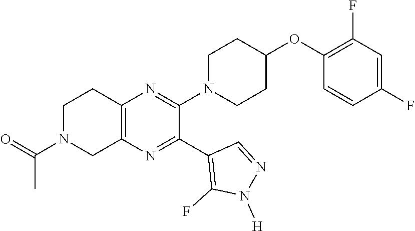 [SMILES: CC(=O)N1CCc2nc(N3CCC(Oc4ccc(F)cc4F)CC3)c(-c3cn[nH]c3F)nc2C1] | 1-{2-[4-(2,4- difluorophenoxy)piperidin- 1-yl]-3-(5-fluoro-1H- pyrazol-4-yl)- 5H,6H,7H,8H-pyrido[3,4- b]pyrazin-6-yl}ethan-1- one | NA |
| 136 | 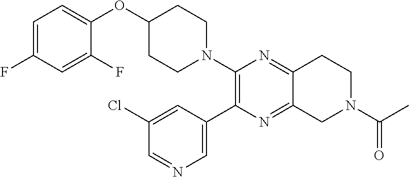 [SMILES: CC(=O)N1CCc2nc(N3CCC(Oc4ccc(F)cc4F)CC3)c(-c3cncc(Cl)c3)nc2C1] | 1-[3-(5-chloropyridin-3- yl)-2-[4-(2,4- difluorophenoxy)piperidin- 1-yl]-5H,6H,7H,8H- pyrido[3,4-b]pyrazin-6- yl]ethan-1-one | NA |
| 137 | 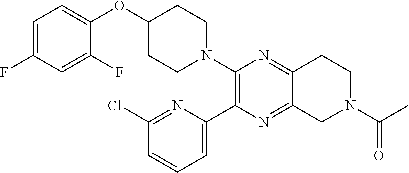 [SMILES: CC(=O)N1CCc2nc(N3CCC(Oc4ccc(F)cc4F)CC3)c(-c3cccc(Cl)n3)nc2C1] | 1-[3-(6-chloropyridin-2- yl)-2-[4-(2,4- difluorophenoxy)piperidin- 1-yl]-5H,6H,7H,8H- pyrido[3,4-b]pyrazin-6- yl]ethan-1-one | NA |
| 138 | 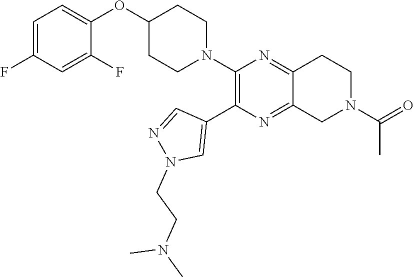 [SMILES: CC(=O)N1CCc2nc(N3CCC(Oc4ccc(F)cc4F)CC3)c(-c3cnn(CCN(C)C)c3)nc2C1] | 1-{2-[4-(2,4- difluorophenoxy)piperidin- 1-yl]-3-{1-[2- (dimethylamino)ethyl]- 1H-pyrazol-4-yl}- 5H,6H,7H,8H-pyrido[3,4- b]pyrazin-6-yl}ethan-1- one | NA |
| 139 | 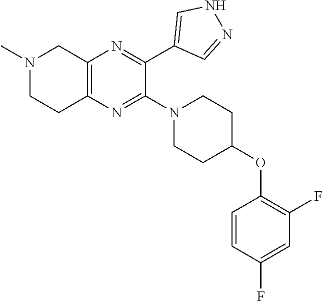 [SMILES: CN1CCc2nc(N3CCC(Oc4ccc(F)cc4F)CC3)c(-c3cn[nH]c3)nc2C1] | 4-(2,4-difluorophenoxy)- 1-[6-methyl-3-(1H- pyrazol-4-yl)- 5H,6H,7H,8H-pyrido[3,4- b]pyrazin-2-yl]piperidine | NA |
| 140 | 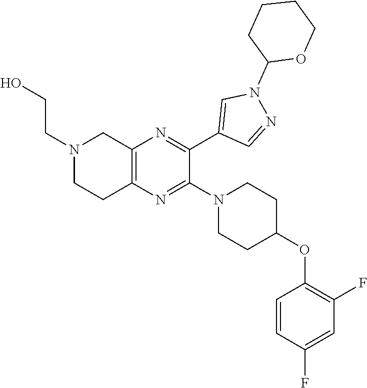 [SMILES: OCCN1CCc2nc(N3CCC(Oc4ccc(F)cc4F)CC3)c(-c3cnn(C4CCCCO4)c3)nc2C1] | 2-{2-[4-(2,4- difluorophenoxy)piperidin- 1-yl]-3-[1-(oxan-2-yl)-1H- pyrazol-4-yl]- 5H,6H,7H,8H-pyrido[3,4- b]pyrazin-6-yl}ethan-1-ol | NA |
| 141 | 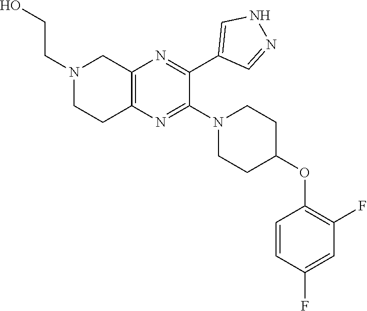 [SMILES: OCCN1CCc2nc(N3CCC(Oc4ccc(F)cc4F)CC3)c(-c3cn[nH]c3)nc2C1] | 2-{2-[4-(2,4- difluorophenoxy)piperidin- 1-yl]-3-(1H-pyrazol-4-yl)- 5H,6H,7H,8H-pyrido[3,4- b]pyrazin-6-yl}ethan-1-ol | NA |
| 142 | 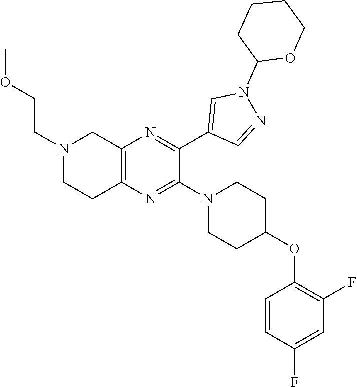 [SMILES: COCCN1CCc2nc(N3CCC(Oc4ccc(F)cc4F)CC3)c(-c3cnn(C4CCCCO4)c3)nc2C1] | 4-(2,4-difluorophenoxy)- 1-[6-(2-methoxyethyl)-3- [1-(oxan-2-yl)-1H-pyrazol- 4-yl]-5H,6H,7H,8H- pyrido[3,4-b]pyrazin-2- yl]piperidine | NA |
| 143 | 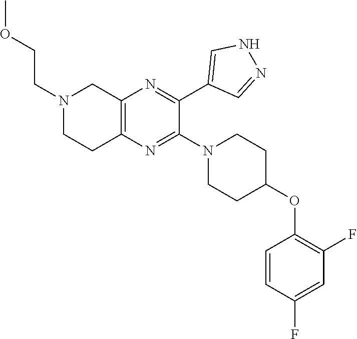 [SMILES: COCCN1CCc2nc(N3CCC(Oc4ccc(F)cc4F)CC3)c(-c3cn[nH]c3)nc2C1] | 4-(2,4-difluorophenoxy)- 1-[6-(2-methoxyethyl)-3- (1H-pyrazol-4-yl)- 5H,6H,7H,8H-pyrido[3,4- b]pyrazin-2-yl]piperidine | NA |
| 144 | 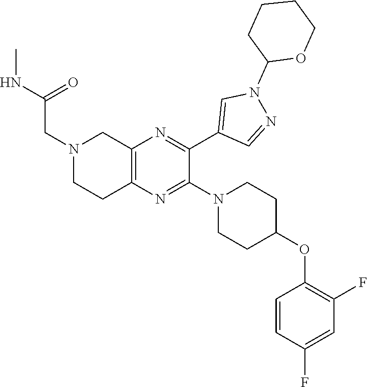 [SMILES: CNC(=O)CN1CCc2nc(N3CCC(Oc4ccc(F)cc4F)CC3)c(-c3cnn(C4CCCCO4)c3)nc2C1] | 2-{2-[4-(2,4- difluorophenoxy)piperidin- 1-yl]-3-[1-(oxan-2-yl)-1H- pyrazol-4-yl]- 5H,6H,7H,8H-pyrido[3,4- b]pyrazin-6-yl}-N- methylacetamide | NA |
| 145 | 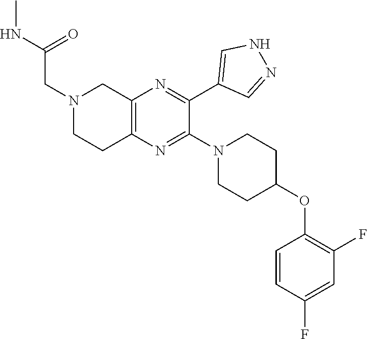 [SMILES: CNC(=O)CN1CCc2nc(N3CCC(Oc4ccc(F)cc4F)CC3)c(-c3cn[nH]c3)nc2C1] | 2-{2-[4-(2,4- difluorophenoxy)piperidin- 1-yl]-3-(1H-pyrazol-4-yl)- 5H,6H,7H,8H-pyrido[3,4- b]pyrazin-6-yl}-N- methylacetamide | NA |
| 146 | 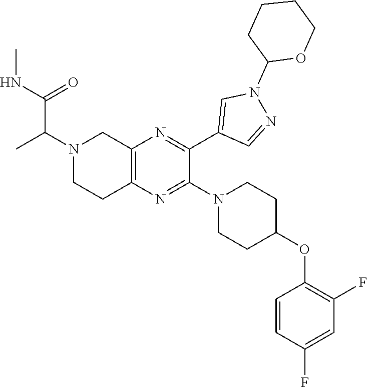 [SMILES: CNC(=O)C(C)N1CCc2nc(N3CCC(Oc4ccc(F)cc4F)CC3)c(-c3cnn(C4CCCCO4)c3)nc2C1] | 2-{2-[4-(2,4- difluorophenoxy)piperidin- 1-yl]-3-[1-(oxan-2-yl)-1H- pyrazol-4-yl]- 5H,6H,7H,8H-pyrido[3,4- b]pyrazin-6-yl}-N- methylpropanamide | NA |
| 147 | 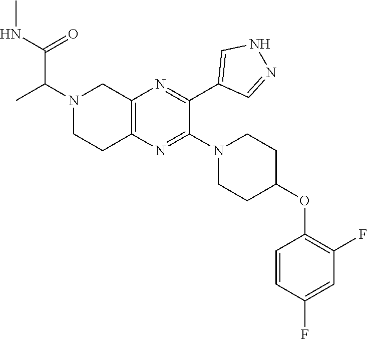 [SMILES: CNC(=O)C(C)N1CCc2nc(N3CCC(Oc4ccc(F)cc4F)CC3)c(-c3cn[nH]c3)nc2C1] | 2-{2-[4-(2,4- difluorophenoxy)piperidin- 1-yl]-3-(1H-pyrazol-4-yl)- 5H,6H,7H,8H-pyrido[3,4- b]pyrazin-6-yl}-N- methylpropanamide | NA |
| 148 | 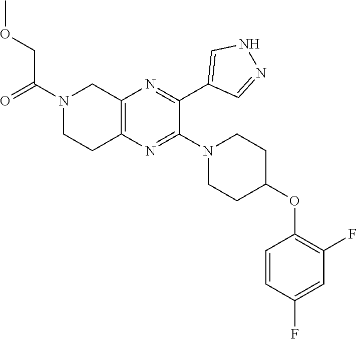 [SMILES: COCC(=O)N1CCc2nc(N3CCC(Oc4ccc(F)cc4F)CC3)c(-c3cn[nH]c3)nc2C1] | 1-{2-[4-(2,4- difluorophenoxy)piperidin- 1-yl]-3-(1H-pyrazol-4-yl)- 5H,6H,7H,8H-pyrido[3,4- b]pyrazin-6-yl}-2- methoxyethan-1-one | NA |
| 149 | 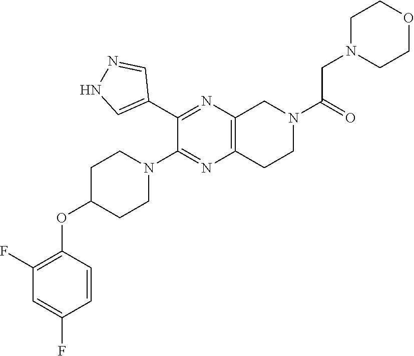 [SMILES: O=C(CN1CCOCC1)N1CCc2nc(N3CCC(Oc4ccc(F)cc4F)CC3)c(-c3cn[nH]c3)nc2C1] | 1-{2-[4-(2,4- difluorophenoxy)piperidin- 1-yl]-3-(1H-pyrazol-4-yl)- 5H,6H,7H,8H-pyrido[3,4- b]pyrazin-6-yl}-2- (morpholin-4-yl)ethan-1- one | NA |
| 150 | 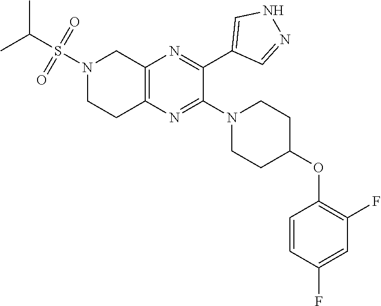 [SMILES: CC(C)S(=O)(=O)N1CCc2nc(N3CCC(Oc4ccc(F)cc4F)CC3)c(-c3cn[nH]c3)nc2C1] | 4-(2,4-difluorophenoxy)- 1-[6-(propane-2-sulfonyl)- 3-(1H-pyrazol-4-yl)- 5H,6H,7H,8H-pyrido[3,4- b]pyrazin-2-yl]piperidine | NA |
| 151 | 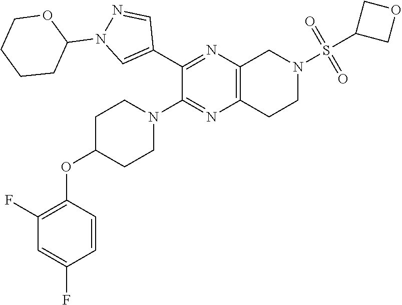 [SMILES: O=S(=O)(C1COC1)N1CCc2nc(N3CCC(Oc4ccc(F)cc4F)CC3)c(-c3cnn(C4CCCCO4)c3)nc2C1] | 4-(2,4-difluorophenoxy)- 1-{3-[1-(oxan-2-yl)-1H- pyrazol-4-yl]-6-(oxetane- 3-sulfonyl)-5H,6H,7H,8H- pyrido[3,4-b]pyrazin-2- yl}piperidine | NA |
| 152 | 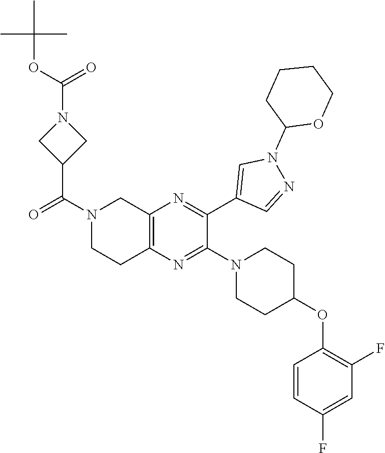 [SMILES: CC(C)(C)OC(=O)N1CC(C(=O)N2CCc3nc(N4CCC(Oc5ccc(F)cc5F)CC4)c(-c4cnn(C5CCCCO5)c4)nc3C2)C1] | tert-butyl 3-{2-[4-(2,4- difluorophenoxy)piperidin- 1-yl]-3-[1-(oxan-2-yl)-1H- pyrazol-4-yl]- 5H,6H,7H,8H-pyrido[3,4- b]pyrazine-6- carbonyl}azetidine-1- carboxylate | NA |
| 153 | 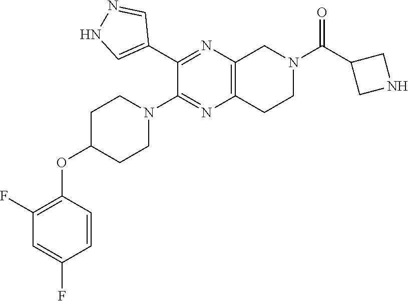 [SMILES: O=C(C1CNC1)N1CCc2nc(N3CCC(Oc4ccc(F)cc4F)CC3)c(-c3cn[nH]c3)nc2C1] | 1-[6-(azetidine-3- carbonyl)-3-(1H-pyrazol- 4-yl)-5H,6H,7H,8H- pyrido[3,4-b]pyrazin-2- yl]-4-(2,4- difluorophenoxy)piperidine | NA |
| 154 | 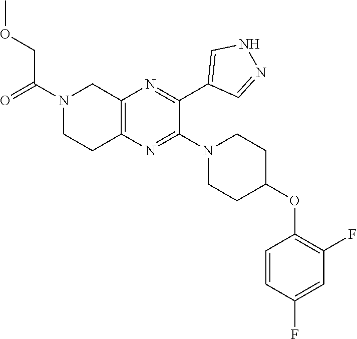 [SMILES: COCC(=O)N1CCc2nc(N3CCC(Oc4ccc(F)cc4F)CC3)c(-c3cn[nH]c3)nc2C1] | methyl 2-[4-(2,4- difluorophenoxy)piperidin- 1-yl]-3-(1H-pyrazol-4-yl)- 5H,6H,7H,8H-pyrido[3,4- b]pyrazine-6-carboxylate | NA |
| 155 | 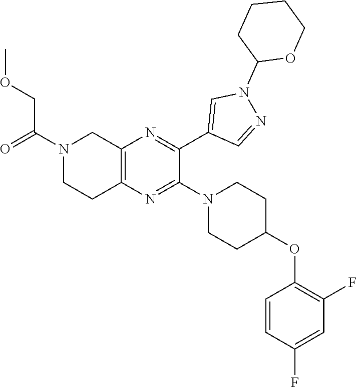 [SMILES: COCC(=O)N1CCc2nc(N3CCC(Oc4ccc(F)cc4F)CC3)c(-c3cnn(C4CCCCO4)c3)nc2C1] | ethyl 2-[4-(2,4- difluorophenoxy)piperidin- 1-yl]-3-[1-(oxan-2-yl)-1H- pyrazol-4-yl]- 5H,6H,7H,8H-pyrido[3,4- b]pyrazine-6-carboxylate | NA |
| 156 | 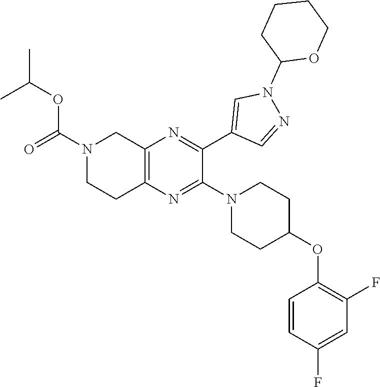 [SMILES: CC(C)OC(=O)N1CCc2nc(N3CCC(Oc4ccc(F)cc4F)CC3)c(-c3cnn(C4CCCCO4)c3)nc2C1] | propan-2-yl 2-[4-(2,4- difluorophenoxy)piperidin- 1-yl]-3-[1-(oxan-2-yl)-1H- pyrazol-4-yl]- 5H,6H,7H,8H-pyrido[3,4- b]pyrazine-6-carboxylate | NA |
| 157 | 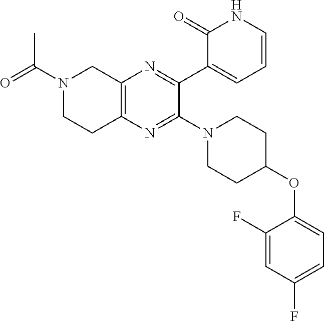 [SMILES: CC(=O)N1CCc2nc(N3CCC(Oc4ccc(F)cc4F)CC3)c(-c3ccc[nH]c3=O)nc2C1] | 3-{6-acetyl-2-[4-(2,4- difluorophenoxy)piperidin- 1-yl]-5H,6H,7H,8H- pyrido[3,4-b]pyrazin-3- yl}-1,2-dihydropyridin-2- one | NA |
| 158 | 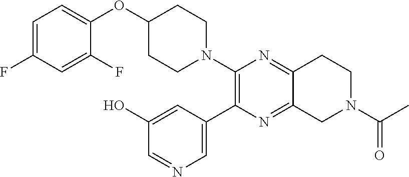 [SMILES: CC(=O)N1CCc2nc(N3CCC(Oc4ccc(F)cc4F)CC3)c(-c3cncc(O)c3)nc2C1] | 1-{2-[4-(2,4- difluorophenoxy)piperidin- 1-yl]-3-(5- hydroxypyridin-3-yl)- 5H,6H,7H,8H-pyrido[3,4- b]pyrazin-6-yl}ethan-1- one | NA |
| 159 | 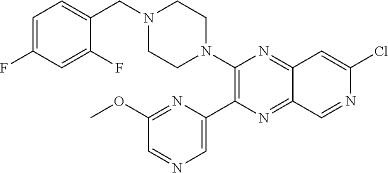 [SMILES: COc1cncc(-c2nc3cnc(Cl)cc3nc2N2CCN(Cc3ccc(F)cc3F)CC2)n1] | 2-(7-chloro-2-{4-[(2,4- difluorophenyl)methyl] piperazin-1-yl}pyrido[3,4- b]pyrazin-3-yl)-6- methoxypyrazine | NA |
| 160 | 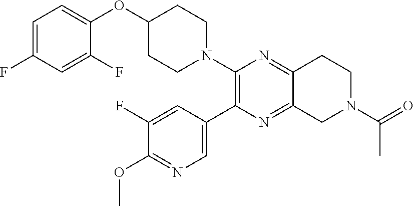 [SMILES: COc1ncc(-c2nc3c(nc2N2CCC(Oc4ccc(F)cc4F)CC2)CCN(C(C)=O)C3)cc1F] | 1-{2-[4-(2,4- difluorophenoxy)piperidin- 1-yl]-3-(5-fluoro-6- methoxypyridin-3-yl)- 5H,6H,7H,8H-pyrido[3,4- b]pyrazin-6-yl}ethan-1- one | NA |
| 161 | 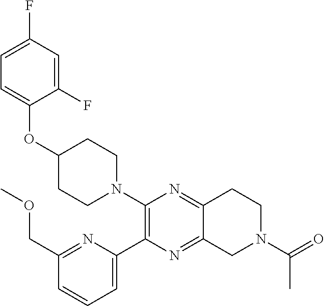 [SMILES: COCc1cccc(-c2nc3c(nc2N2CCC(Oc4ccc(F)cc4F)CC2)CCN(C(C)=O)C3)n1] | 1-{2-[4-(2,4- difluorophenoxy)piperidin- 1-yl]-3-[6- (methoxymethyl)pyridin- 2-yl]-5H,6H,7H,8H- pyrido[3,4-b]pyrazin-6- yl}ethan-1-one | NA |
| 162 | 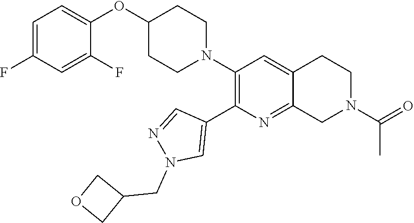 [SMILES: CC(=O)N1CCc2cc(N3CCC(Oc4ccc(F)cc4F)CC3)c(-c3cnn(CC4COC4)c3)nc2C1] | 1-{2-[4-(2,4- difluorophenoxy)piperidin- 1-yl]-3-{1-[(oxetan-3- yl)methyl]-1H-pyrazol-4- yl}-5H,6H,7H,8H- pyrido[3,4-b]pyrazin-6- yl}ethan-1-one | NA |
| 163 | 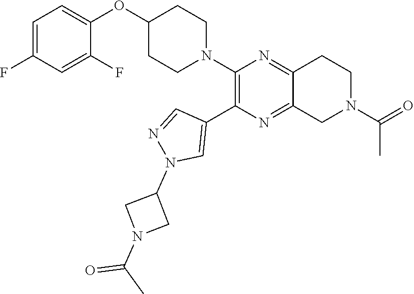 [SMILES: CC(=O)N1CCc2nc(N3CCC(Oc4ccc(F)cc4F)CC3)c(-c3cnn(C4CN(C(C)=O)C4)c3)nc2C1] | 1-[3-(4-{6-acetyl-2-[4- (2,4- difluorophenoxy)piperidin- 1-yl]-5H,6H,7H,8H- pyrido[3,4-b]pyrazin-3- yl}-1H-pyrazol-1- yl)azetidin-1-yl]ethan-1- one | NA |
| 164 | 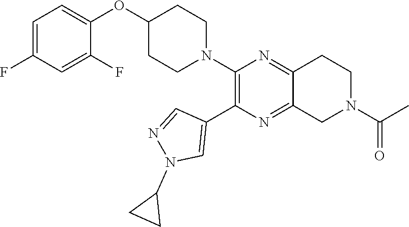 [SMILES: CC(=O)N1CCc2nc(N3CCC(Oc4ccc(F)cc4F)CC3)c(-c3cnn(C4CC4)c3)nc2C1] | 1-[3-(1-cyclopropyl-1H- pyrazol-4-yl)-2-[4-(2,4- difluorophenoxy)piperidin- 1-yl]-5H,6H,7H,8H- pyrido[3,4-b]pyrazin-6- yllethan-1-one | NA |
| 165 | 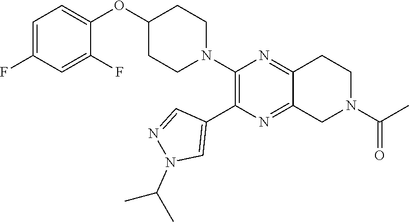 [SMILES: CC(=O)N1CCc2nc(N3CCC(Oc4ccc(F)cc4F)CC3)c(-c3cnn(C(C)C)c3)nc2C1] | 1-{2-[4-(2,4- difluorophenoxy )piperidin- 1-yl]-3-[1-(propan-2-yl)- 1H-pyrazol-4-yl]- 5H,6H,7H,8H-pyrido[3,4- b]pyrazin-6-yl}ethan-1-one | NA |
| 166 | 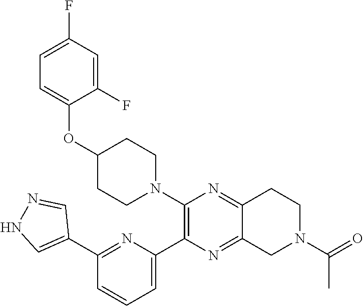 [SMILES: CC(=O)N1CCc2nc(N3CCC(Oc4ccc(F)cc4F)CC3)c(-c3cccc(-c4cn[nH]c4)n3)nc2C1] | 1-{2-[4-(2,4- difluorophenoxy)piperidin- 1-yl]-3-[6-(1H-pyrazol-4- yl)pyridin-2-yl]- 5H,6H,7H,8H-pyrido[3,4- b]pyrazin-6-yl}ethan-1- one | NA |
| 167 | 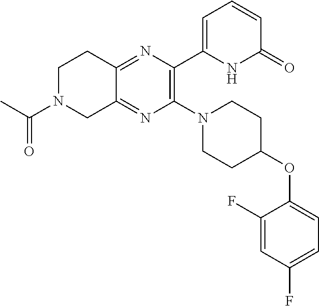 [SMILES: CC(=O)N1CCc2nc(-c3cccc(=O)[nH]3)c(N3CCC(Oc4ccc(F)cc4F)CC3)nc2C1] | 6-{6-acetyl-3-[4-(2,4- difluorophenoxy)piperidin- 1-yl]-5H,6H,7H,8H- pyrido[3,4-b]pyrazin-2- yl}-1,2-dihydropyridin-2- one | NA |
| 168 | 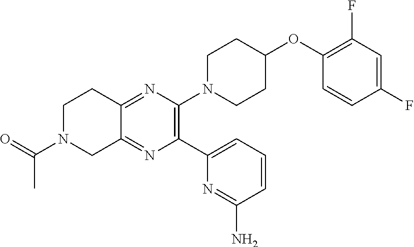 [SMILES: CC(=O)N1CCc2nc(N3CCC(Oc4ccc(F)cc4F)CC3)c(-c3cccc(N)n3)nc2C1] | 1-[3-(6-aminopyridin-2- yl)-2-[4-(2,4- difluorophenoxy)piperidin- 1-yl]-5H,6H,7H,8H- pyrido[3,4-b]pyrazin-6- yl]ethan-1-one | NA |
| 169 | 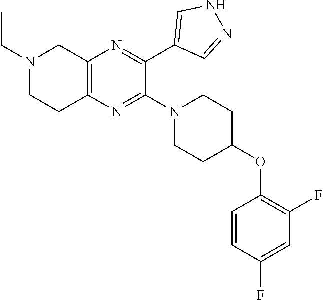 [SMILES: CCN1CCc2nc(N3CCC(Oc4ccc(F)cc4F)CC3)c(-c3cn[nH]c3)nc2C1] | 4-(2,4-difluorophenoxy)- 1-{6-methyl-3-[1-(oxan-2- yl)-1H-pyrazol-4-yl]- 5H,6H,7H,8H-pyrido[3,4- b]pyrazin-2-yl}piperidine | NA |
| 170 | 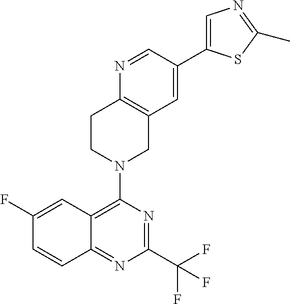 [SMILES: Cc1ncc(-c2cnc3c(c2)CN(c2nc(C(F)(F)F)nc4ccc(F)cc24)CC3)s1] | 4-(2,4-difluorophenoxy)- 1-[6-ethyl-3-(1H-pyrazol- 4-yl)-5H,6H,7H,8H- pyrido[3,4-b]pyrazin-2- yl]piperidine | NA |
| 171 | 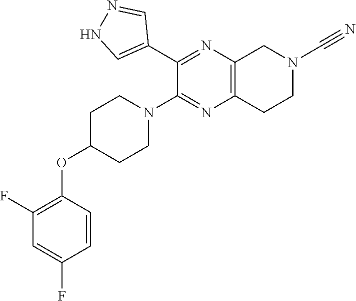 [SMILES: N#CN1CCc2nc(N3CCC(Oc4ccc(F)cc4F)CC3)c(-c3cn[nH]c3)nc2C1] | 2-[4-(2,4- difluorophenoxy)piperidin- 1-yl]-3-(1H-pyrazol-4-yl)- 5H,6H,7H,8H-pyrido[3,4- b]pyrazine-6-carbonitrile | NA |
| 172 | 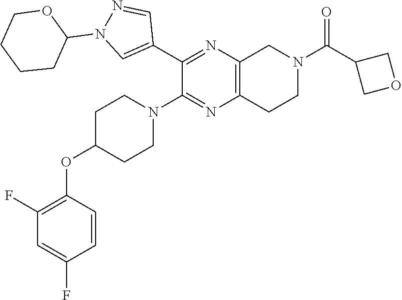 [SMILES: O=C(C1COC1)N1CCc2nc(N3CCC(Oc4ccc(F)cc4F)CC3)c(-c3cnn(C4CCCCO4)c3)nc2C1] | 4-(2,4-difluorophenoxy)- 1-{3-[1-(oxan-2-yl)-1H- pyrazol-4-yl]-6-(oxetane- 3-carbonyl)-5H,6H,7H,8H- pyrido[3,4-b]pyrazin-2- yl}piperidine | NA |
| 173 | 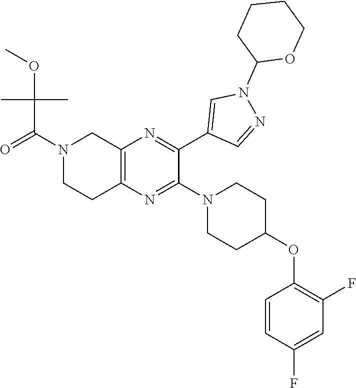 [SMILES: COC(C)(C)C(=O)N1CCc2nc(N3CCC(Oc4ccc(F)cc4F)CC3)c(-c3cnn(C4CCCCO4)c3)nc2C1] | 1-{2-[4-(2,4- difluorophenoxy)piperidin- 1-yl]-3-[1-(oxan-2-yl)-1H- pyrazol-4-yl]- 5H,6H,7H,8H-pyrido[3,4- b]pyrazin-6-yl}-2- methoxy-2-methylpropan- 1-one | NA |
| 174 | 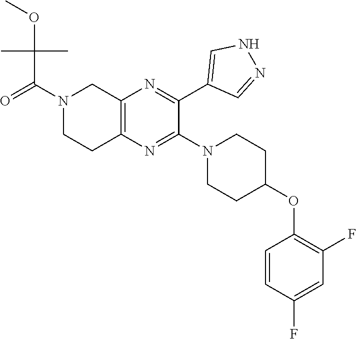 [SMILES: COC(C)(C)C(=O)N1CCc2nc(N3CCC(Oc4ccc(F)cc4F)CC3)c(-c3cn[nH]c3)nc2C1] | 1-{2-[4-(2,4- difluorophenoxy)piperidin- 1-yl]-3-(1H-pyrazol-4-yl)- 5H,6H,7H,8H-pyrido[3,4- b]pyrazin-6-yl}-2- methoxy-2-methylpropan- 1-one | NA |
| 175 | 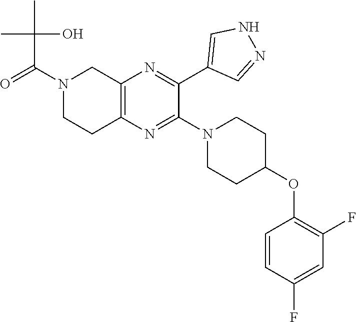 [SMILES: CC(C)(O)C(=O)N1CCc2nc(N3CCC(Oc4ccc(F)cc4F)CC3)c(-c3cn[nH]c3)nc2C1] | 1-{2-[4-(2,4- difluorophenoxy)piperidin- 1-yl]-3-(1H-pyrazol-4-yl)- 5H,6H,7H,8H-pyrido[3,4- b]pyrazin-6-yl}-2- hydroxy-2-methylpropan- 1-one | NA |
| 176 | 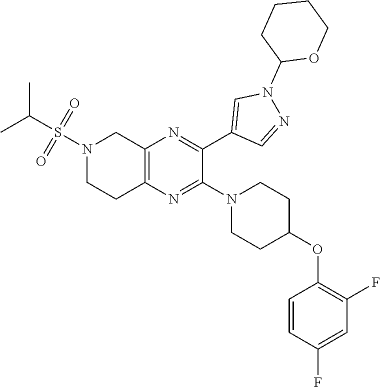 [SMILES: CC(C)S(=O)(=O)N1CCc2nc(N3CCC(Oc4ccc(F)cc4F)CC3)c(-c3cnn(C4CCCCO4)c3)nc2C1] | 4-(2,4-difluorophenoxy)- 1-{3-[1-(oxan-2-yl)-1H- pyrazol-4-yl]-6-(propane- 2-sulfonyl)-5H,6H,7H,8H- pyrido[3,4-b]pyrazin-2- yl}piperidine | NA |
| 177 | 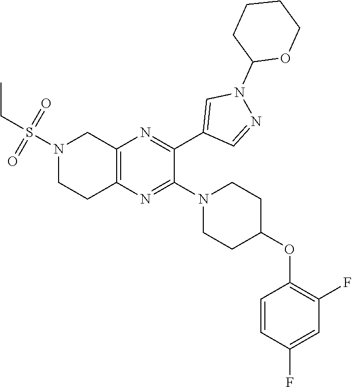 [SMILES: CCS(=O)(=O)N1CCc2nc(N3CCC(Oc4ccc(F)cc4F)CC3)c(-c3cnn(C4CCCCO4)c3)nc2C1] | 4-(2,4-difluorophenoxy)- 1-[6-(ethanesulfonyl)-3- [1-(oxan-2-yl)-1H-pyrazol- 4-yl]-5H,6H,7H,8H- pyrido[3,4-b]pyrazin-2- yl]piperidine | NA |
| 178 | 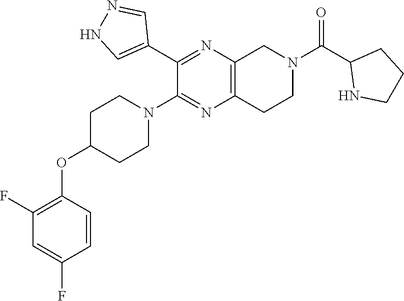 [SMILES: O=C(C1CCCN1)N1CCc2nc(N3CCC(Oc4ccc(F)cc4F)CC3)c(-c3cn[nH]c3)nc2C1] | 4-(2,4-difluorophenoxy)- 1-[3-(1H-pyrazol-4-yl)-6- (pyrrolidine-2-carbonyl)- 5H,6H,7H,8H-pyrido[3,4- b]pyrazin-2-yl]piperidine | NA |
| 179 | 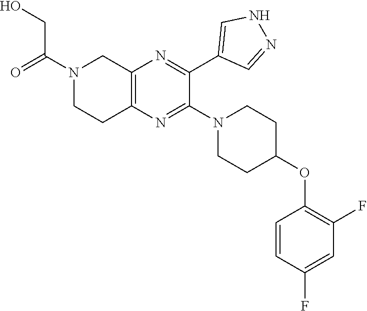 [SMILES: O=C(CO)N1CCc2nc(N3CCC(Oc4ccc(F)cc4F)CC3)c(-c3cn[nH]c3)nc2C1] | 1-{2-[4-(2,4- difluorophenoxy)piperidin- 1-yl]-3-(1H-pyrazol-4-yl)- 5H,6H,7H,8H-pyrido[3,4- b]pyrazin-6-yl}-2- hydroxyethan-1-one | NA |
| 180 | 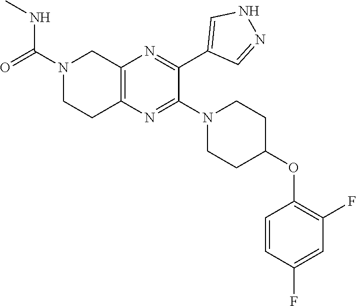 [SMILES: CNC(=O)N1CCc2nc(N3CCC(Oc4ccc(F)cc4F)CC3)c(-c3cn[nH]c3)nc2C1] | 2-[4-(2,4- difluorophenoxy)piperidin- 1-yl]-N-methyl-3-(1H- pyrazol-4-yl)- 5H,6H,7H,8H-pyrido[3,4- b]pyrazine-6-carboxamide | NA |
| 181 | 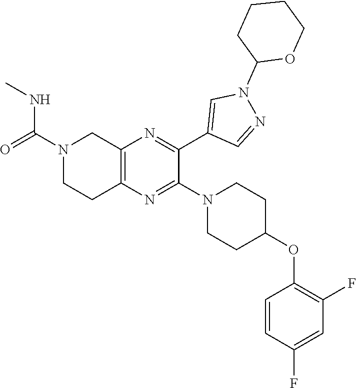 [SMILES: CNC(=O)N1CCc2nc(N3CCC(Oc4ccc(F)cc4F)CC3)c(-c3cnn(C4CCCCO4)c3)nc2C1] | 2-[4-(2,4- difluorophenoxy)piperidin- 1-yl]-N-methyl-3-[1- (oxan-2-yl)-1H-pyrazol-4- yl]-5H,6H,7H,8H- pyrido[3,4-b]pyrazine-6- carboxamide | NA |
| 182 | 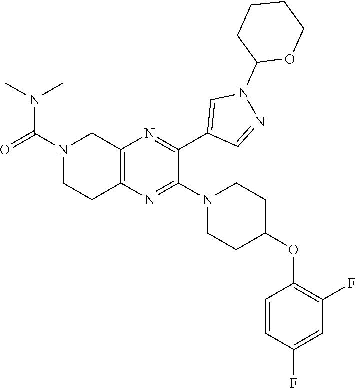 [SMILES: CN(C)C(=O)N1CCc2nc(N3CCC(Oc4ccc(F)cc4F)CC3)c(-c3cnn(C4CCCCO4)c3)nc2C1] | 2-[4-(2,4- difluorophenoxy)piperidin- 1-yl]-N,N-dimethyl-3-[1- (oxan-2-yl)-1H-pyrazol-4- yl]-5H,6H,7H,8H- pyrido[3,4-b]pyrazine-6- carboxamide | NA |
| 183 | 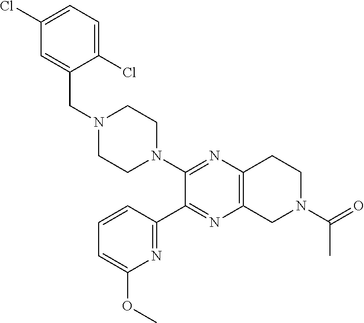 [SMILES: COc1cccc(-c2nc3c(nc2N2CCN(Cc4cc(Cl)ccc4Cl)CC2)CCN(C(C)=O)C3)n1] | 1-(2-{4-[(2,5- dichlorophenyl)methyl] piperazin-1-yl}-3-(6- methoxypyridin-2-yl)- 5H,6H,7H,8H-pyrido[3,4- b]pyrazin-6-yl)ethan-1- one | NA |
| 184 | 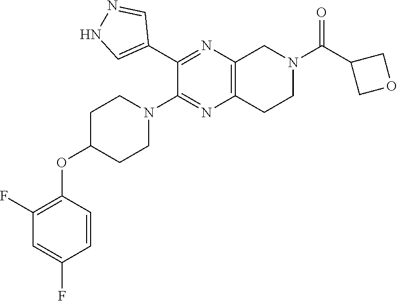 [SMILES: O=C(C1COC1)N1CCc2nc(N3CCC(Oc4ccc(F)cc4F)CC3)c(-c3cn[nH]c3)nc2C1] | 4-(2,4-difluorophenoxy)- 1-[6-(oxetane-3- carbonyl)-3-(1H-pyrazol- 4-yl)-5H,6H,7H,8H- pyrido[3,4-b]pyrazin-2- yl]piperidine | NA |
| 185 | 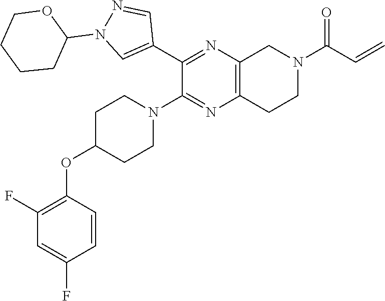 [SMILES: C=CC(=O)N1CCc2nc(N3CCC(Oc4ccc(F)cc4F)CC3)c(-c3cnn(C4CCCCO4)c3)nc2C1] | 1-{2-[4-(2,4- difluorophenoxy)piperidin- 1-yl]-3-[1-(oxan-2-yl)-1H- pyrazol-4-yl]- 5H,6H,7H,8H-pyrido[3,4- b]pyrazin-6-yl}prop-2-en- 1-one | NA |
| 186 | 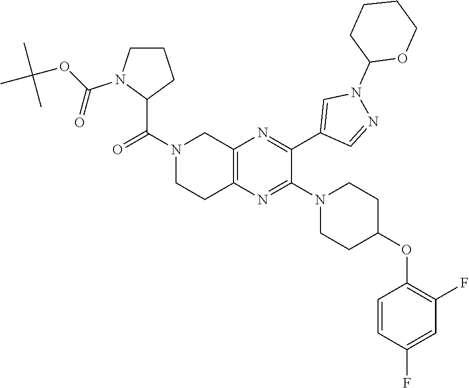 [SMILES: CC(C)(C)OC(=O)N1CCCC1C(=O)N1CCc2nc(N3CCC(Oc4ccc(F)cc4F)CC3)c(-c3cnn(C4CCCCO4)c3)nc2C1] | tert-butyl 2-{2-[4-(2,4- difluorophenoxy)piperidin- 1-yl]-3-[1-(oxan-2-yl)-1H- pyrazol-4-yl]- 5H,6H,7H,8H-pyrido[3,4- b]pyrazine-6- carbonyl}pyrrolidine-1- carboxylate | NA |
| 187 | 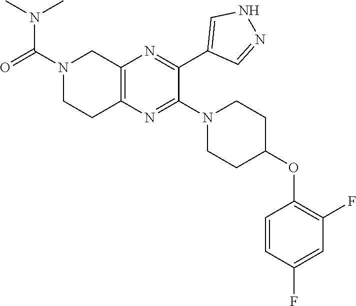 [SMILES: CN(C)C(=O)N1CCc2nc(N3CCC(Oc4ccc(F)cc4F)CC3)c(-c3cn[nH]c3)nc2C1] | 2-[4-(2,4- difluorophenoxy)piperidin- 1-yl]-N,N-dimethyl-3- (1H-pyrazol-4-yl)- 5H,6H,7H,8H-pyrido[3,4- b]pyrazine-6-carboxamide | NA |
| 188 | 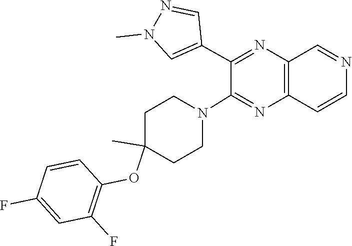 [SMILES: Cn1cc(-c2nc3cnccc3nc2N2CCC(C)(Oc3ccc(F)cc3F)CC2)cn1] | 4-(2,4-difluorophenoxy)- 4-methyl-1-[3-(1-methyl- 1H-pyrazol-4- yl)pyrido[3,4-b]pyrazin-2- yl]piperidine | NA |
| 189 | 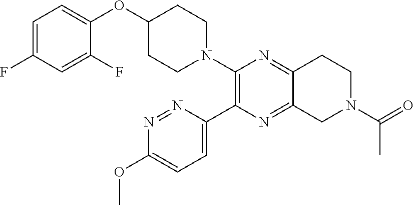 [SMILES: COc1ccc(-c2nc3c(nc2N2CCC(Oc4ccc(F)cc4F)CC2)CCN(C(C)=O)C3)nn1] | 1-{2-[4-(2,4- difluorophenoxy)piperidin- 1-yl]-3-(6- methoxypyridazin-3-yl)- 5H,6H,7H,8H-pyrido[3,4- b]pyrazin-6-yl}ethan-1- one | NA |
| 190 | 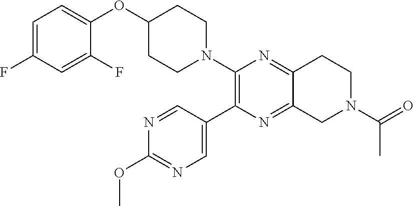 [SMILES: COc1ncc(-c2nc3c(nc2N2CCC(Oc4ccc(F)cc4F)CC2)CCN(C(C)=O)C3)cn1] | 1-{2-[4-(2,4- difluorophenoxy)piperidin- 1-yl]-3-(2- methoxypyrimidin-5-yl)- 5H,6H,7H,8H-pyrido[3,4- b]pyrazin-6-yl}ethan-1- one | NA |
| 191 | 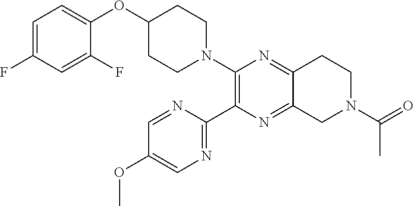 [SMILES: COc1cnc(-c2nc3c(nc2N2CCC(Oc4ccc(F)cc4F)CC2)CCN(C(C)=O)C3)nc1] | 1-{2-[4-(2,4- difluorophenoxy)piperidin- 1-yl]-3-(5- methoxypyrimidin-2-yl)- 5H,6H,7H,8H-pyrido[3,4- b]pyrazin-6-yl}ethan-1- one | NA |
| 192 | 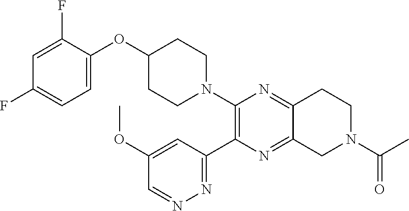 [SMILES: COc1cnnc(-c2nc3c(nc2N2CCC(Oc4ccc(F)cc4F)CC2)CCN(C(C)=O)C3)c1] | 1-{2-[4-(2,4- difluorophenoxy)piperidin- 1-yl]-3-(5- methoxypyridazin-3-yl)- 5H,6H,7H,8H-pyrido[3,4- b]pyrazin-6-yl}ethan-1- one | NA |
| 193 | 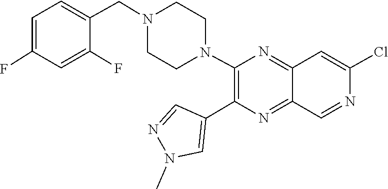 [SMILES: Cn1cc(-c2nc3cnc(Cl)cc3nc2N2CCN(Cc3ccc(F)cc3F)CC2)cn1] | 1-[7-chloro-3-(1-methyl- 1H-pyrazol-4- yl)pyrido[3,4-b]pyrazin-2- yl]-4-[(2,4- difluorophenyl)methyl] piperazine | NA |
| 194 | 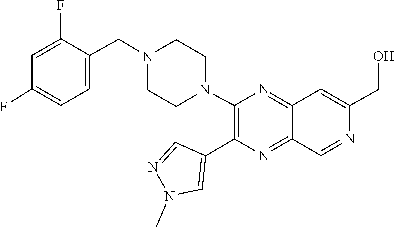 [SMILES: Cn1cc(-c2nc3cnc(CO)cc3nc2N2CCN(Cc3ccc(F)cc3F)CC2)cn1] | (2-{4-[(2,4- difluorophenyl)methyl] piperazin-1-yl}-3-(1-methyl- 1H-pyrazol-4- yl)pyrido[3,4-b]pyrazin-7- yl)methanol | NA |
| 195 | 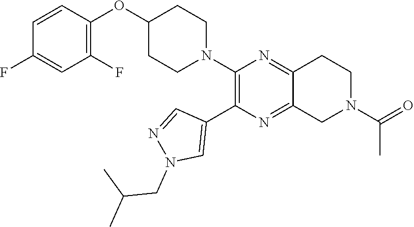 [SMILES: CC(=O)N1CCc2nc(N3CCC(Oc4ccc(F)cc4F)CC3)c(-c3cnn(CC(C)C)c3)nc2C1] | 1-{2-[4-(2,4- difluorophenoxy)piperidin- 1-yl]-3-[1-(2- methylpropyl)-1H- pyrazol-4-yl]- 5H,6H,7H,8H-pyrido[3,4- b]pyrazin-6-yl}ethan-1- one | NA |
| 196 | 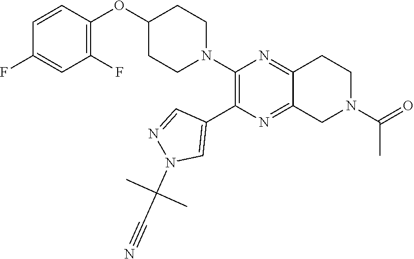 [SMILES: CC(=O)N1CCc2nc(N3CCC(Oc4ccc(F)cc4F)CC3)c(-c3cnn(C(C)(C)C#N)c3)nc2C1] | 2-(4-{6-acetyl-2-[4-(2,4- difluorophenoxy)piperidin- 1-yl]-5H,6H,7H,8H- pyrido[3,4-b]pyrazin-3- yl}-1H-pyrazol-1-yl)-2- methylpropanenitrile | NA |
| 197 | 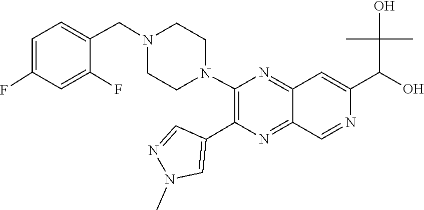 [SMILES: Cn1cc(-c2nc3cnc(C(O)C(C)(C)O)cc3nc2N2CCN(Cc3ccc(F)cc3F)CC2)cn1] | 1-(2-{4-[(2,4- difluorophenyl)methyl] piperazin-1-yl}-3-(1-methyl- 1H-pyrazol-4- yl)pyrido[3,4-b]pyrazin-7- yl)-2-methylpropane-1,2- diol | NA |
| 198 | 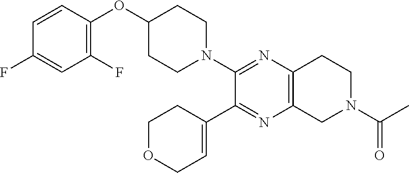 [SMILES: CC(=O)N1CCc2nc(N3CCC(Oc4ccc(F)cc4F)CC3)c(C3=CCOCC3)nc2C1] | 1-{2-[4-(2,4- difluorophenoxy)piperidin- 1-yl]-3-(3,6-dihydro-2H- pyran-4-yl)-5H,6H,7H,8H- pyrido[3,4-b]pyrazin-6- yl}ethan-1-one | NA |
| 199 | 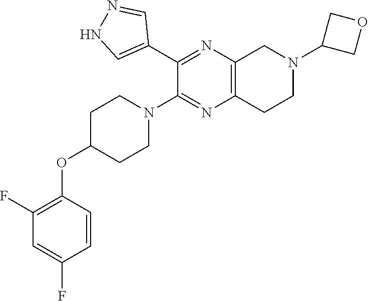 [SMILES: Fc1ccc(OC2CCN(c3nc4c(nc3-c3cn[nH]c3)CN(C3COC3)CC4)CC2)c(F)c1] | 4-(2,4-difluorophenoxy)- 1-[6-(oxetan-3-yl)-3-(1H- pyrazol-4-yl)- 5H,6H,7H,8H-pyrido[3,4- b]pyrazin-2-yl]piperidine | NA |
| 200 | 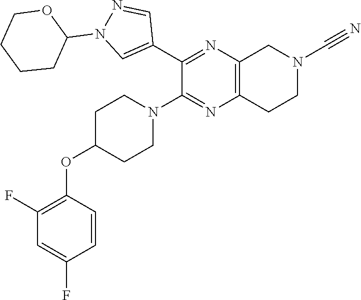 [SMILES: N#CN1CCc2nc(N3CCC(Oc4ccc(F)cc4F)CC3)c(-c3cnn(C4CCCCO4)c3)nc2C1] | 2-[4-(2,4- difluorophenoxy)piperidin- 1-yl]-3-[1-(oxan-2-yl)-1H- pyrazol-4-yl]- 5H,6H,7H,8H-pyrido[3,4- b]pyrazine-6-carbonitrile | NA |
| 201 | 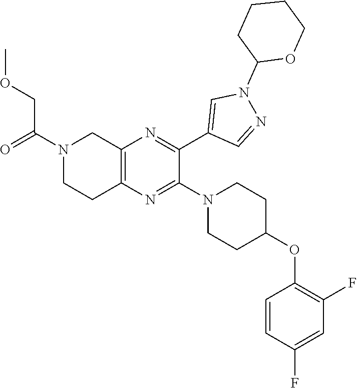 [SMILES: COCC(=O)N1CCc2nc(N3CCC(Oc4ccc(F)cc4F)CC3)c(-c3cnn(C4CCCCO4)c3)nc2C1] | 1-{2-[4-(2,4- difluorophenoxy)piperidin- 1-yl]-3-[1-(oxan-2-yl)-1H- pyrazol-4-yl]- 5H,6H,7H,8H-pyrido[3,4- b]pyrazin-6-yl}-2- methoxyethan-1-one | NA |
| 202 | 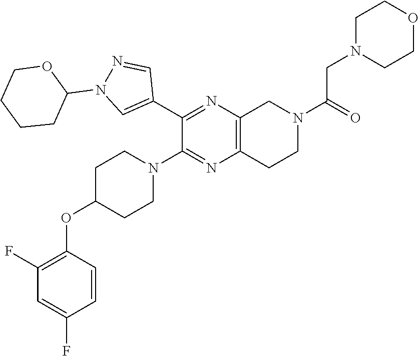 [SMILES: O=C(CN1CCOCC1)N1CCc2nc(N3CCC(Oc4ccc(F)cc4F)CC3)c(-c3cnn(C4CCCCO4)c3)nc2C1] | 1-{2-[4-(2,4- difluorophenoxy)piperidin- 1-yl]-3-[1-(oxan-2-yl)-1H- pyrazol-4-yl]- 5H,6H,7H,8H-pyrido[3,4- b]pyrazin-6-yl}-2- (morpholin-4-yl)ethan-1- one | NA |
| 203 | 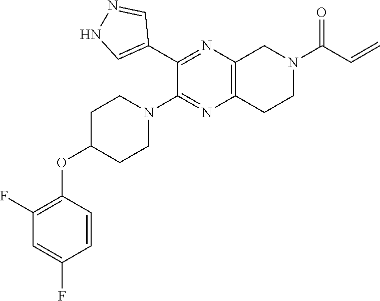 [SMILES: C=CC(=O)N1CCc2nc(N3CCC(Oc4ccc(F)cc4F)CC3)c(-c3cn[nH]c3)nc2C1] | 1-{2-[4-(2,4- difluorophenoxy)piperidin- 1-yl]-3-(1H-pyrazol-4-yl)- 5H,6H,7H,8H-pyrido[3,4- b]pyrazin-6-yl}prop-2-en- 1-one | NA |
| 204 | 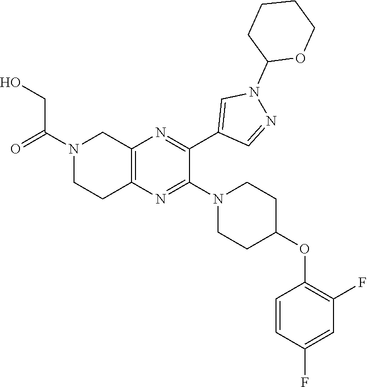 [SMILES: O=C(CO)N1CCc2nc(N3CCC(Oc4ccc(F)cc4F)CC3)c(-c3cnn(C4CCCCO4)c3)nc2C1] | 1-{2-[4-(2,4- difluorophenoxy)piperidin- 1-yl]-3-[1-(oxan-2-yl)-1H- pyrazol-4-yl]- 5H,6H,7H,8H-pyrido [3,4- b]pyrazin-6-yl}-2- hydroxyethan-1-one | NA |
| 205 | 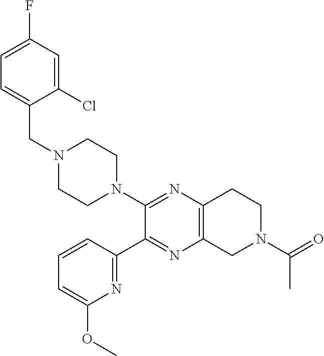 [SMILES: COc1cccc(-c2nc3c(nc2N2CCN(Cc4ccc(F)cc4Cl)CC2)CCN(C(C)=O)C3)n1] | 1-(2-{4-[(2-chloro-4- fluorophenyl)methyl] piperazin-1-yl}-3-(6- methoxypyridin-2-yl)- 5H,6H,7H,8H-pyrido[3,4- b]pyrazin-6-yl)ethan-1- one | NA |
| 206 | 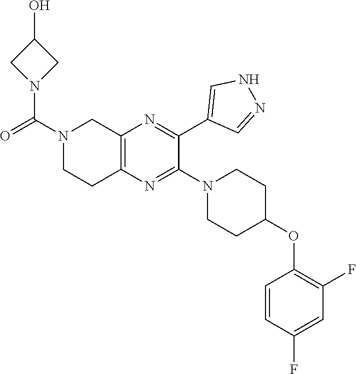 [SMILES: O=C(N1CCc2nc(N3CCC(Oc4ccc(F)cc4F)CC3)c(-c3cn[nH]c3)nc2C1)N1CC(O)C1] | 1-{2-[4-(2,4- difluorophenoxy)piperidin- 1-yl]-3-(1H-pyrazol-4-yl)- 5H,6H,7H,8H-pyrido[3,4- b]pyrazine-6- carbonyl}azetidin-3-ol | NA |
| 207 | 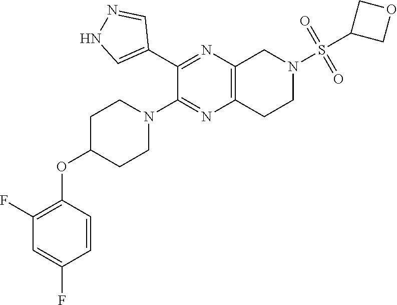 [SMILES: O=S(=O)(C1COC1)N1CCc2nc(N3CCC(Oc4ccc(F)cc4F)CC3)c(-c3cn[nH]c3)nc2C1] | 4-(2,4-difluorophenoxy)- 1-[6-(oxetane-3-sulfonyl)- 3-(1H-pyrazol-4-yl)- 5H,6H,7H,8H-pyrido[3,4- b]pyrazin-2-yl]piperidine | NA |
| 208 | 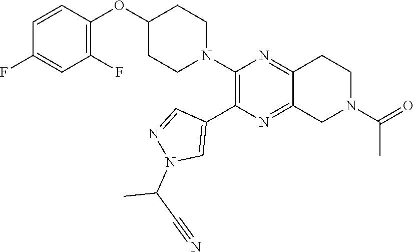 [SMILES: CC(=O)N1CCc2nc(N3CCC(Oc4ccc(F)cc4F)CC3)c(-c3cnn(C(C)C#N)c3)nc2C1] | 2-(4-{6-acetyl-2-[4-(2,4- difluorophenoxy)piperidin- 1-yl]-5H,6H,7H,8H- pyrido[3,4-b]pyrazin-3- yl}-1H-pyrazol-1- yl)propanenitrile | NA |
| 209 | 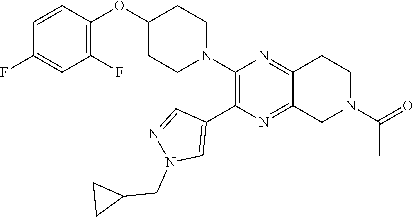 [SMILES: CC(=O)N1CCc2nc(N3CCC(Oc4ccc(F)cc4F)CC3)c(-c3cnn(CC4CC4)c3)nc2C1] | 1-{3-[1- (cyclopropylmethyl)-1H- pyrazol-4-yl]-2-[4-(2,4- difluorophenoxy)piperidin- 1-yl]-5H,6H,7H,8H- pyrido[3,4-b]pyrazin-6- yl}ethan-1-one | NA |
| 210 | 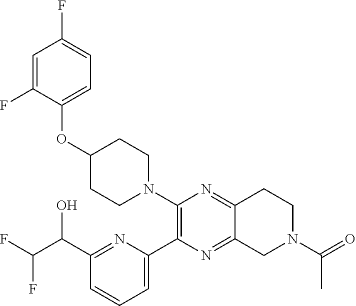 [SMILES: CC(=O)N1CCc2nc(N3CCC(Oc4ccc(F)cc4F)CC3)c(-c3cccc(C(O)C(F)F)n3)nc2C1] | 1-{3-[6-(2,2-difluoro-1- hydroxyethyl)pyridin-2- yl]-2-[4-(2,4- difluorophenoxy)piperidin- 1-yl]-5H,6H,7H,8H- pyrido[3,4-b]pyrazin-6- yl}ethan-1-one | NA |
| 211 | 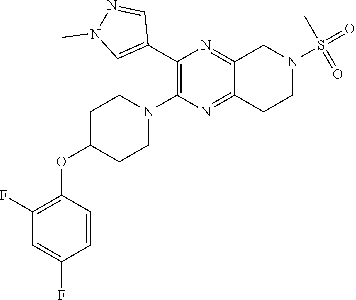 [SMILES: Cn1cc(-c2nc3c(nc2N2CCC(Oc4ccc(F)cc4F)CC2)CCN(S(C)(=O)=O)C3)cn1] | 4-(2,4-difluorophenoxy)- 1-[6-methanesulfonyl-3- (1-methyl-1H-pyrazol-4- yl)-5H,6H,7H,8H- pyrido[3,4-b]pyrazin-2- yl]piperidine | NA |
| 212 | 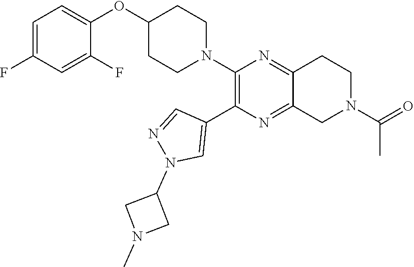 [SMILES: CC(=O)N1CCc2nc(N3CCC(Oc4ccc(F)cc4F)CC3)c(-c3cnn(C4CN(C)C4)c3)nc2C1] | 1-{2-[4-(2,4- difluorophenoxy)piperidin- 1-yl]-3-[1-(1- methylazetidin-3-yl)-1H- pyrazol-4-yl]- 5H,6H,7H,8H-pyrido[3,4- b]pyrazin-6-yl}ethan-1- one | NA |
| 213 | 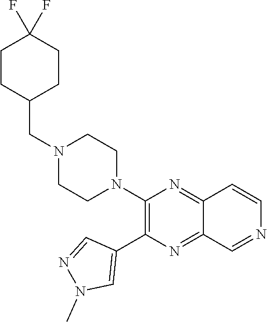 [SMILES: Cn1cc(-c2nc3cnccc3nc2N2CCN(CC3CCC(F)(F)CC3)CC2)cn1] | 1-[(4,4- difluorocyclohexyl)methyl ]-4-[3-(1-methyl-1H- pyrazol-4-yl)pyrido[3,4- b]pyrazin-2-yl]piperazine | NA |
| 214 | 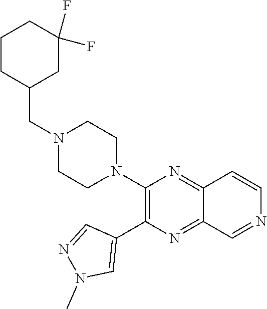 [SMILES: Cn1cc(-c2nc3cnccc3nc2N2CCN(CC3CCCC(F)(F)C3)CC2)cn1] | 1-[(3,3- difluorocyclohexyl)methyl ]-4-[3-(1-methyl-1H- pyrazol-4-yl)pyrido[3,4- b]pyrazin-2-yl]piperazine | NA |
| 215 | 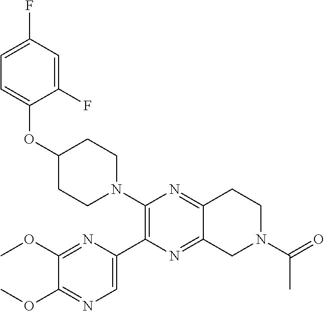 [SMILES: COc1ncc(-c2nc3c(nc2N2CCC(Oc4ccc(F)cc4F)CC2)CCN(C(C)=O)C3)nc1OC] | 1-{2-[4-(2,4- difluorophenoxy)piperidin- 1-yl]-3-(5,6- dimethoxypyrazin-2-yl)- 5H,6H,7H,8H-pyrido[3,4- b]pyrazin-6-yl}ethan-1- one | NA |
| 216 | 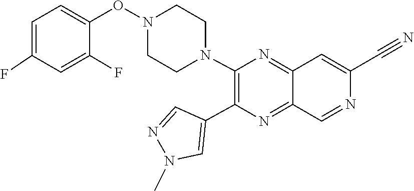 [SMILES: Cn1cc(-c2nc3cnc(C#N)cc3nc2N2CCN(Oc3ccc(F)cc3F)CC2)cn1] | 2-{4-[(2,4- difluorophenyl)methyl] piperazin-1-yl}-3-(1-methyl- 1H-pyrazol-4- yl)pyrido[3,4-b]pyrazine- 7-carbonitrile | NA |
| 217 | 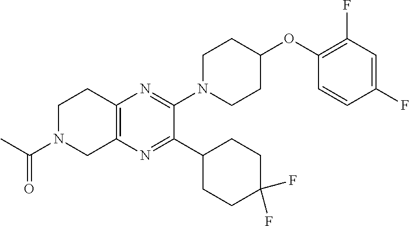 [SMILES: CC(=O)N1CCc2nc(N3CCC(Oc4ccc(F)cc4F)CC3)c(C3CCC(F)(F)CC3)nc2C1] | 1-[3-(4,4- difluorocyclohexyl)-2-[4- (2,4- difluorophenoxy)piperidin- 1-yl]-5H,6H,7H,8H- pyrido[3,4-b]pyrazin-6- yl]ethan-1-one | NA |
| 218 | 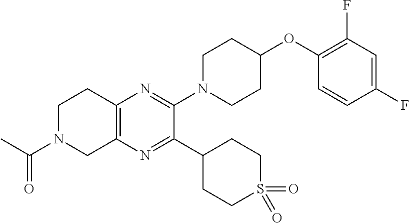 [SMILES: CC(=O)N1CCc2nc(N3CCC(Oc4ccc(F)cc4F)CC3)c(C3CCS(=O)(=O)CC3)nc2C1] | 4-{6-acetyl-2-[4-(2,4- difluorophenoxy)piperidin- 1-yl]-5H,6H,7H,8H- pyrido[3,4-b]pyrazin-3- yl}-1λ⁶-thiane-1,1-dione | NA |
| 219 | 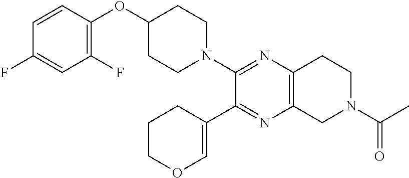 [SMILES: CC(=O)N1CCc2nc(N3CCC(Oc4ccc(F)cc4F)CC3)c(C3=COCCC3)nc2C1] | 1-{2-[4-(2,4- difluorophenoxy)piperidin- 1-yl]-3-(3,4-dihydro-2H- pyran-5-yl)-5H,6H,7H,8H- pyrido[3,4-b]pyrazin-6- yl}ethan-1-one | NA |
| 220 | 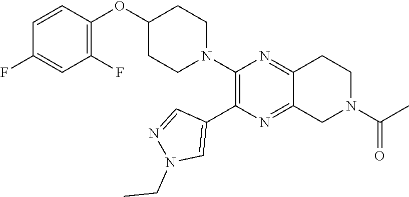 [SMILES: CCn1cc(-c2nc3c(nc2N2CCC(Oc4ccc(F)cc4F)CC2)CCN(C(C)=O)C3)cn1] | 1-{2-[4-(2,4- difluorophenoxy)piperidin- 1-yl]-3-(1-ethyl-1H- pyrazol-4-yl)- 5H,6H,7H,8H-pyrido[3,4- b]pyrazin-6-yl}ethan-1- one | NA |
| 221 | 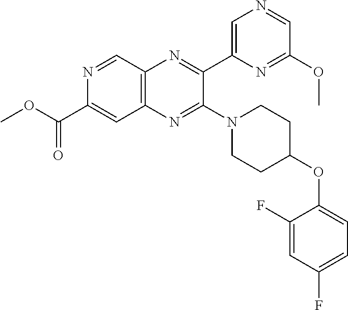 [SMILES: COC(=O)c1cc2nc(N3CCC(Oc4ccc(F)cc4F)CC3)c(-c3cncc(OC)n3)nc2cn1] | methyl 2-[4-(2,4- difluorophenoxy)piperidin- 1-yl]-3-(6- methoxypyrazin-2- yl)pyrido[3,4-b]pyrazine- 7-carboxylate | NA |
| 222 | 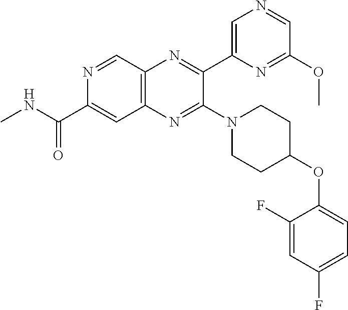 [SMILES: CNC(=O)c1cc2nc(N3CCC(Oc4ccc(F)cc4F)CC3)c(-c3cncc(OC)n3)nc2cn1] | 2-[4-(2,4- difluorophenoxy)piperidin- 1-yl]-3-(6- methoxypyrazin-2-yl)-N- methylpyrido[3,4- b]pyrazine-7-carboxamide | NA |
| 223 | 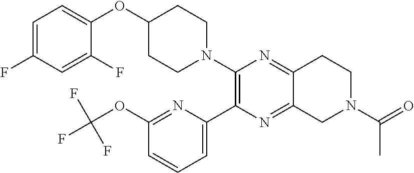 [SMILES: CC(=O)N1CCc2nc(N3CCC(Oc4ccc(F)cc4F)CC3)c(-c3cccc(OC(F)(F)F)n3)nc2C1] | 1-{2-[4-(2,4- difluorophenoxy)piperidin- 1-yl]-3-[6- (trifluoromethoxy)pyridin- 2-yl]-5H,6H,7H,8H- pyrido[3,4-b]pyrazin-6- yl}ethan-1-one | NA |
| 224 | 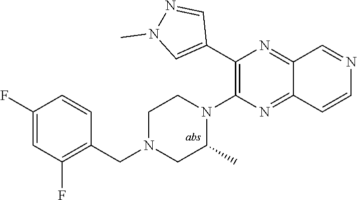 [SMILES: C[C@@H]1CN(Cc2ccc(F)cc2F)CCN1c1nc2ccncc2nc1-c1cnn(C)c1] , or enantiomer | (2R)-4-[(2,4- difluorophenyl)methyl]-2- methyl-1-[3-(1-methyl- 1H-pyrazol-4- yl)pyrido[3,4-b]pyrazin-2- yl]piperazine, or enantiomer | NA |
| 225 | or enantiomer 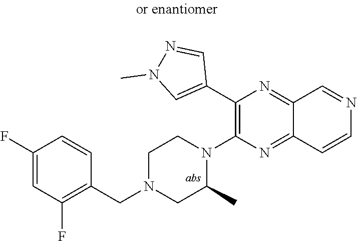 [SMILES: C[C@H]1CN(Cc2ccc(F)cc2F)CCN1c1nc2ccncc2nc1-c1cnn(C)c1] | (2S)-4-[(2,4- difluorophenyl)methyl]-2- methyl-1-[3-(1-methyl- 1H-pyrazol-4- yl)pyrido[3,4-b]pyrazin-2- yl]piperazine, or enantiomer | NA |
| 226 | 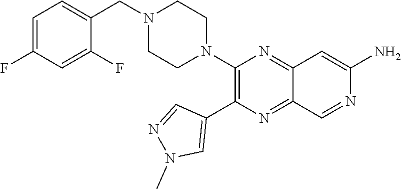 [SMILES: Cn1cc(-c2nc3cnc(N)cc3nc2N2CCN(Cc3ccc(F)cc3F)CC2)cn1] | 2-{4-[(2,4- difluorophenyl)methyl] piperazin-1-yl}-3-(1-methyl- 1H-pyrazol-4- yl)pyrido[3,4-b]pyrazin-7- amine | NA |
| 227 | 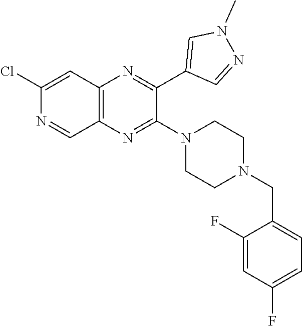 [SMILES: Cn1cc(-c2nc3cc(Cl)ncc3nc2N2CCN(Cc3ccc(F)cc3F)CC2)cn1] | 1-[7-chloro-2-(1-methyl- 1H-pyrazol-4- yl)pyrido[3,4-b]pyrazin-3- yl]-4-[(2,4- difluorophenyl)methyl] piperazine | NA |
| 228 | 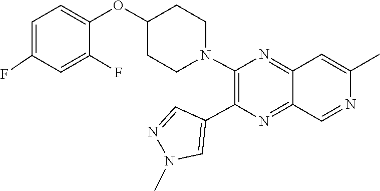 [SMILES: Cc1cc2nc(N3CCC(Oc4ccc(F)cc4F)CC3)c(-c3cnn(C)c3)nc2cn1] | 4-(2,4-difluorophenoxy)- 1-[7-methyl-3-(1-methyl- 1H-pyrazol-4- yl)pyrido[3,4-b]pyrazin-2- yl]piperidine | NA |
| 229 | 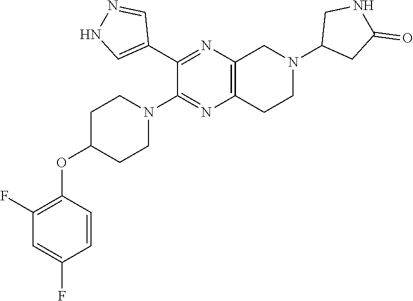 [SMILES: O=C1CC(N2CCc3nc(N4CCC(Oc5ccc(F)cc5F)CC4)c(-c4cn[nH]c4)nc3C2)CN1] | 4-{2-[4-(2,4- difluorophenoxy)piperidin- 1-yl]-3-(1H-pyrazol-4-yl)- 5H,6H,7H,8H-pyrido[3,4- b]pyrazin-6-yl}pyrrolidin- 2-one | NA |
| 230 | 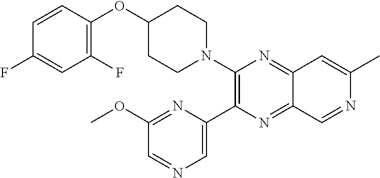 [SMILES: COc1cncc(-c2nc3cnc(C)cc3nc2N2CCC(Oc3ccc(F)cc3F)CC2)n1] | 2-{2-[4-(2,4- difluorophenoxy)piperidin- 1-yl]-7-methylpyrido[3,4- b]pyrazin-3-yl}-6- methoxypyrazine | NA |
| 231 | 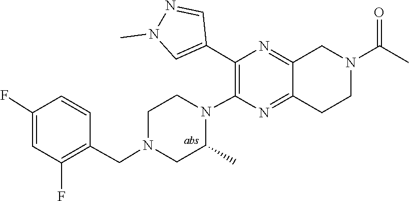 [SMILES: CC(=O)N1CCc2nc(N3CCN(Cc4ccc(F)cc4F)C[C@H]3C)c(-c3cnn(C)c3)nc2C1] or enantiomer | 1-{2-[(2R)-4-[(2,4- difluorophenyl)methyl]-2- methylpiperazin-1-yl]-3- (1-methyl-1H-pyrazol-4- yl)-5H,6H,7H,8H- pyrido[3,4-b]pyrazin-6- yl}ethan-1-one, or enantiomer | NA |
| 232 | 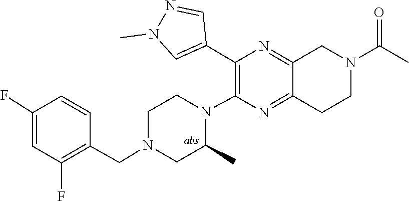 [SMILES: CC(=O)N1CCc2nc(N3CCN(Cc4ccc(F)cc4F)C[C@@H]3C)c(-c3cnn(C)c3)nc2C1] , or enantiomer | 1-{2-[(2S)-4-[(2,4- difluorophenyl)methyl]-2- methylpiperazin-1-yl]-3- (1-methyl-1H-pyrazol-4- yl)-5H,6H,7H,8H- pyrido[3,4-b]pyrazin-6- yl}ethan-1-one, or enantiomer | NA |
| 233 | 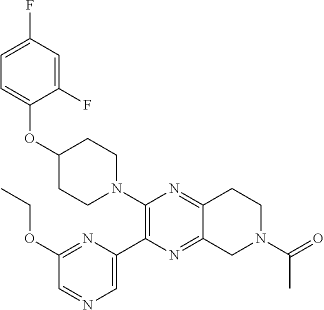 [SMILES: CCOc1cncc(-c2nc3c(nc2N2CCC(Oc4ccc(F)cc4F)CC2)CCN(C(C)=O)C3)n1] | 1-{2-[4-(2,4- difluorophenoxy)piperidin- 1-yl]-3-(6-ethoxypyrazin- 2-yl)-5H,6H,7H,8H- pyrido[3,4-b]pyrazin-6- yl}ethan-1-one | NA |
| 234 | 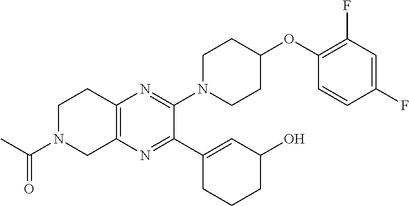 [SMILES: CC(=O)N1CCc2nc(N3CCC(Oc4ccc(F)cc4F)CC3)c(C3=CC(O)CCC3)nc2C1] | 1-{2-[4-(2,4- difluorophenoxy)piperidin- 1-yl]-3-(3- hydroxycyclohex-1-en-1- yl)-5H,6H,7H,8H- pyrido[3,4-b]pyrazin-6- yl}ethan-1-one | NA |
| 235 | 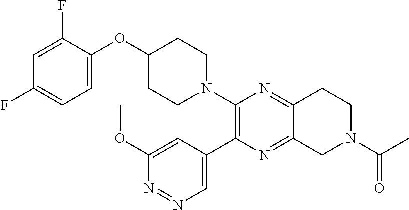 [SMILES: COc1cc(-c2nc3c(nc2N2CCC(Oc4ccc(F)cc4F)CC2)CCN(C(C)=O)C3)cnn1] | 1-{2-[4-(2,4- difluorophenoxy)piperidin- 1-yl]-3-(6- methoxypyridazin-4-yl)- 5H,6H,7H,8H-pyrido[3,4- b]pyrazin-6-yl}ethan-1- one | NA |
| 236 | 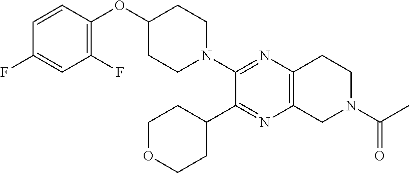 [SMILES: CC(=O)N1CCc2nc(N3CCC(Oc4ccc(F)cc4F)CC3)c(C3CCOCC3)nc2C1] | 1-{2-[4-(2,4- difluorophenoxy)piperidin- 1-yl]-3-(oxan-4-yl)- 5H,6H,7H,8H-pyrido[3,4- b]pyrazin-6-yl}ethan-1- one | NA |
| 237 | 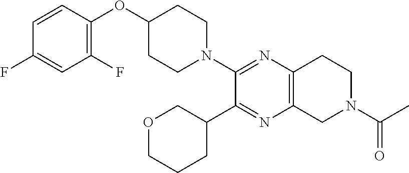 [SMILES: CC(=O)N1CCc2nc(N3CCC(Oc4ccc(F)cc4F)CC3)c(C3CCCOC3)nc2C1] | 1-{2-[4-(2,4- difluorophenoxy)piperidin- 1-yl]-3-(oxan-3-yl)- 5H,6H,7H,8H-pyrido[3,4- b]pyrazin-6-yl}ethan-1- one | NA |
| 238 | 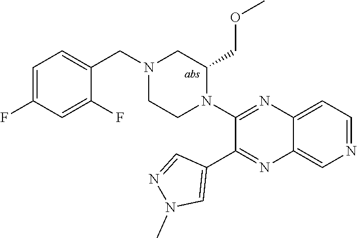 [SMILES: COC[C@H]1CN(Cc2ccc(F)cc2F)CCN1c1nc2ccncc2nc1-c1cnn(C)c1] , or enantiomer | (2R)-4-[(2,4- difluorophenyl)methyl]-2- (methoxymethyl)-1-[3-(1- methyl-1H-pyrazol-4- yl)pyrido[3,4-b]pyrazin-2- yl]piperazine, or enantiomer | NA |
| 239 | 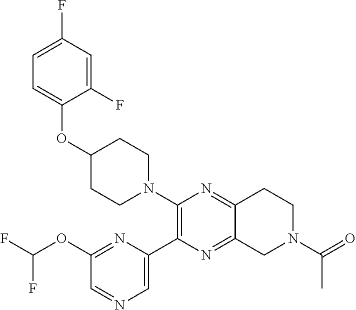 [SMILES: CC(=O)N1CCc2nc(N3CCC(Oc4ccc(F)cc4F)CC3)c(-c3cncc(OC(F)F)n3)nc2C1] | 1-{3-[6- (difluoromethoxy)pyrazin- 2-yl]-2-[4-(2,4- difluorophenoxy)piperidin- 1-yl]-5H,6H,7H,8H- pyrido[3,4-b]pyrazin-6- yl}ethan-1-one | NA |
| 240 | 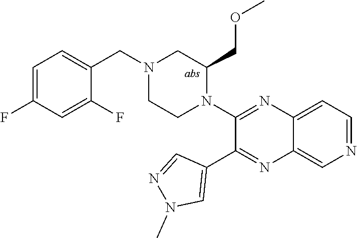 [SMILES: COC[C@@H]1CN(Cc2ccc(F)cc2F)CCN1c1nc2ccncc2nc1-c1cnn(C)c1] or enantiomer | (2S)-4-[(2,4- difluorophenyl)methyl]-2- (methoxymethyl)-1-[3-(1- methyl-1H-pyrazol-4- yl)pyrido[3,4-b]pyrazin-2- yl]piperazine, or enantiomer | NA |
| 241 | 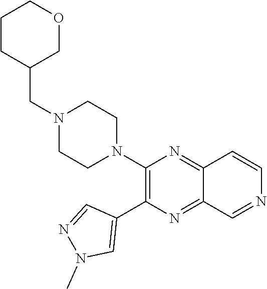 [SMILES: Cn1cc(-c2nc3cnccc3nc2N2CCN(CC3CCCOC3)CC2)cn1] | 1-[3-(1-methyl-1H- pyrazol-4-yl)pyrido[3,4- b]pyrazin-2-yl]-4-[(oxan- 3-yl)methyl]piperazine | NA |
| 242 | 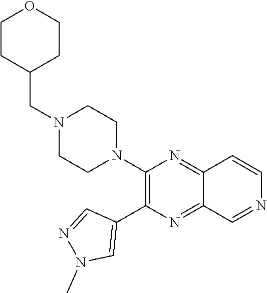 [SMILES: Cn1cc(-c2nc3cnccc3nc2N2CCN(CC3CCOCC3)CC2)cn1] | 1-[3-(1-methyl-1H- pyrazol-4-yl)pyrido[3,4- b]pyrazin-2-yl]-4-[(oxan- 4-yl)methyl]piperazine | NA |
| 243 | 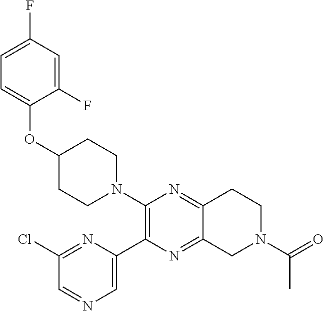 [SMILES: CC(=O)N1CCc2nc(N3CCC(Oc4ccc(F)cc4F)CC3)c(-c3cncc(Cl)n3)nc2C1] | 1-[3-(6-chloropyrazin-2- yl)-2-[4-(2,4- difluorophenoxy)piperidin- 1-yl]-5H,6H,7H,8H- pyrido[3,4-b]pyrazin-6- yllethan-1-one | NA |
| 244 | 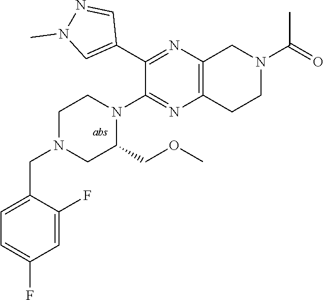 [SMILES: COC[C@@H]1CN(Cc2ccc(F)cc2F)CCN1c1nc2c(nc1-c1cnn(C)c1)CN(C(C)=O)CC2] or enantiomer | 1-{2-[(2S)-4-[(2,4- difluorophenyl)methyl]-2- (methoxymethyl)piperazin- 1-yl]-3-(1-methyl-1H- pyrazol-4-yl)- 5H,6H,7H,8H-pyrido[3,4- b]pyrazin-6-yl}ethan-1- one, or enantiomer | NA |
| 245 | 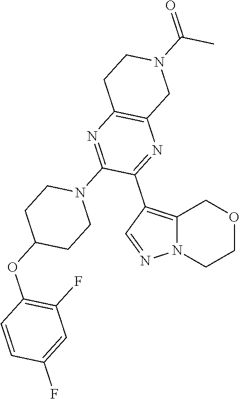 [SMILES: CC(=O)N1CCc2nc(N3CCC(Oc4ccc(F)cc4F)CC3)c(-c3cnn4c3COCC4)nc2C1] | 1-{2-[4-(2,4- difluorophenoxy)piperidin- 1-yl]-3-{4H,6H,7H- pyrazolo [3,2- c][1,4]oxazin-3-yl}- 5H,6H,7H,8H-pyrido[3,4- b]pyrazin-6-yl}ethan-1- one | NA |
| 246 | 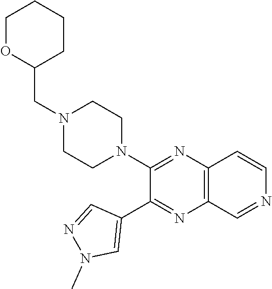 [SMILES: Cn1cc(-c2nc3cnccc3nc2N2CCN(CC3CCCCO3)CC2)cn1] | 1-[3-(1-methyl-1H- pyrazol-4-yl)pyrido[3,4- b]pyrazin-2-yl]-4-[(oxan- 2-yl)methyl]piperazine | NA |
| 247 | 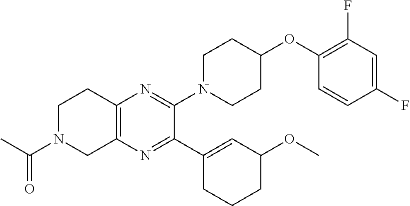 [SMILES: COC1C=C(c2nc3c(nc2N2CCC(Oc4ccc(F)cc4F)CC2)CCN(C(C)=O)C3)CCC1] | 1-{2-[4-(2,4- difluorophenoxy)piperidin- 1-yl]-3-(3- methoxycyclohex-1-en-1- yl)-5H,6H,7H,8H- pyrido[3,4-b]pyrazin-6- yl}ethan-1-one | NA |
| 248 | 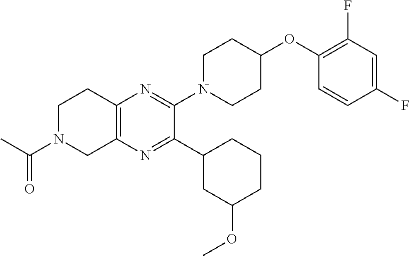 [SMILES: COC1CCCC(c2nc3c(nc2N2CCC(Oc4ccc(F)cc4F)CC2)CCN(C(C)=O)C3)C1] | 1-{2-[4-(2,4- difluorophenoxy)piperidin- 1-yl]-3-(3- methoxycyclohexyl)- 5H,6H,7H,8H-pyrido[3,4- b]pyrazin-6-yl}ethan-1- one | NA |
| 249 | 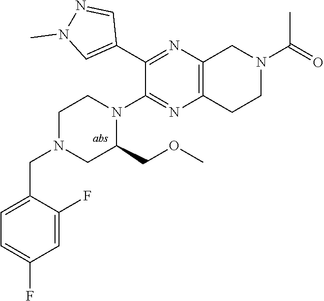 [SMILES: COC[C@H]1CN(Cc2ccc(F)cc2F)CCN1c1nc2c(nc1-c1cnn(C)c1)CN(C(C)=O)CC2] , or enantiomer | 1-{2-[(2R)-4-[(2,4- difluorophenyl)methyl]-2- (methoxymethyl)piperazin- 1-yl]-3-(1-methyl-1H- pyrazol-4-yl)- 5H,6H,7H,8H-pyrido[3,4- b]pyrazin-6-yl}ethan-1- one, or enantiomer | NA |
| 250 | 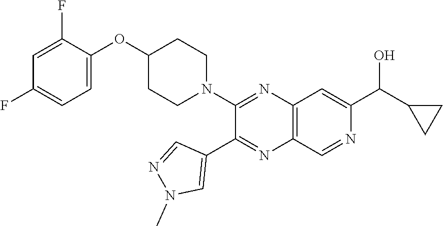 [SMILES: Cn1cc(-c2nc3cnc(C(O)C4CC4)cc3nc2N2CCC(Oc3ccc(F)cc3F)CC2)cn1] | cyclopropyl({2-[4-(2,4- difluorophenoxy)piperidin- 1-yl]-3-(1-methyl-1H- pyrazol-4-yl)pyrido[3,4- b]pyrazin-7-yl})methanol | NA |
| 251 | 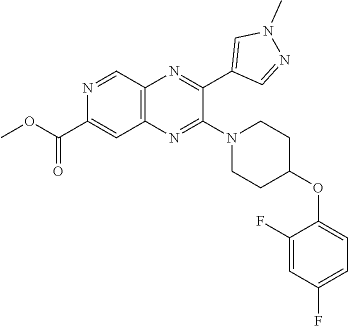 [SMILES: COC(=O)c1cc2nc(N3CCC(Oc4ccc(F)cc4F)CC3)c(-c3cnn(C)c3)nc2cn1] | methyl 2-[4-(2,4- difluorophenoxy)piperidin- 1-yl]-3-(1-methyl-1H- pyrazol-4-yl)pyrido[3,4- b]pyrazine-7-carboxylate | NA |
| 252 | 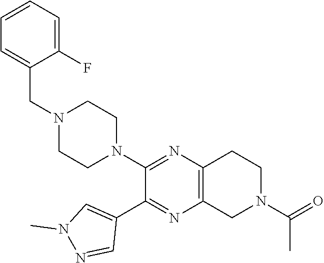 [SMILES: CC(=O)N1CCc2nc(N3CCN(Cc4ccccc4F)CC3)c(-c3cnn(C)c3)nc2C1] | 1-(2-{4-[(2- fluorophenyl)methyl] piperazin-1-yl}-3-(1-methyl- 1H-pyrazol-4-yl)- 5H,6H,7H,8H-pyrido[3,4- b]pyrazin-6-yl)ethan-1- one | NA |
| 253 | 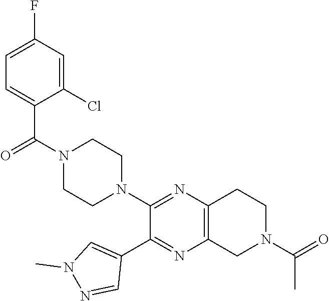 [SMILES: CC(=O)N1CCc2nc(N3CCN(C(=O)c4ccc(F)cc4Cl)CC3)c(-c3cnn(C)c3)nc2C1] | 1-{2-[4-(2-chloro-4- fluorobenzoyl)piperazin- 1-yl]-3-(1-methyl-1H- pyrazol-4-yl)- 5H,6H,7H,8H-pyrido[3,4- b]pyrazin-6-yl}ethan-1- one | NA |
| 254 | 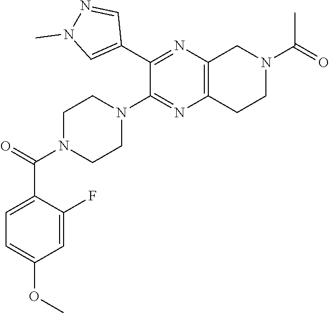 [SMILES: COc1ccc(C(=O)N2CCN(c3nc4c(nc3-c3cnn(C)c3)CN(C(C)=O)CC4)CC2)c(F)c1] | 1-{2-[4-(2-fluoro-4- methoxybenzoyl)piperazin- 1-yl]-3-(1-methyl-1H- pyrazol-4-yl)- 5H,6H,7H,8H-pyrido[3,4- b]pyrazin-6-yl}ethan-1- one | NA |
| 255 | 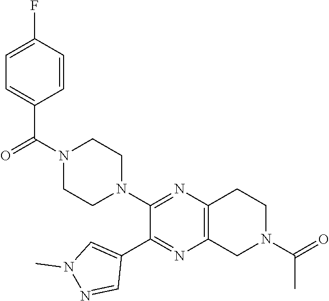 [SMILES: CC(=O)N1CCc2nc(N3CCN(C(=O)c4ccc(F)cc4)CC3)c(-c3cnn(C)c3)nc2C1] | 1-{2-[4-(4- fluorobenzoyl)piperazin- 1-yl]-3-(1-methyl-1H- pyrazol-4-yl)- 5H,6H,7H,8H-pyrido[3,4- b]pyrazin-6-yl}ethan-1- one | NA |
| 256 | 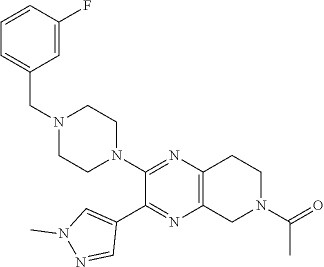 [SMILES: CC(=O)N1CCc2nc(N3CCN(Cc4cccc(F)c4)CC3)c(-c3cnn(C)c3)nc2C1] | 1-(2-{4-[(3- fluorophenyl)methyl] piperazin-1-yl}-3-(1-methyl- 1H-pyrazol-4-yl)- 5H,6H,7H,8H-pyrido[3,4- b]pyrazin-6-yl)ethan-1- one | NA |
| 257 | 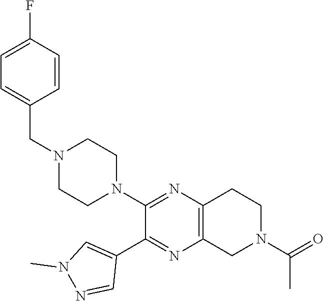 [SMILES: CC(=O)N1CCc2nc(N3CCN(Cc4ccc(F)cc4)CC3)c(-c3cnn(C)c3)nc2C1] | 1-(2-{4-[(4- fluorophenyl)methyl] piperazin-1-yl}-3-(1-methyl- 1H-pyrazol-4-yl)- 5H,6H,7H,8H-pyrido[3,4- b]pyrazin-6-yl)ethan-1- one | NA |
| 258 | 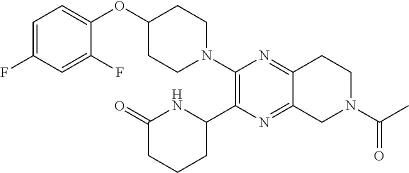 [SMILES: CC(=O)N1CCc2nc(N3CCC(Oc4ccc(F)cc4F)CC3)c(C3CCCC(=O)N3)nc2C1] | 6-{6-acetyl-2-[4-(2,4- difluorophenoxy)piperidin- 1-yl]-5H,6H,7H,8H- pyrido[3,4-b]pyrazin-3- yl}piperidin-2-one | NA |
| 259 | 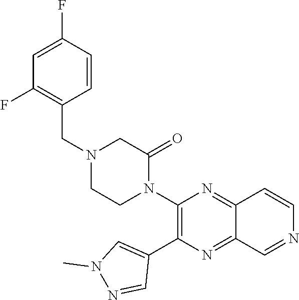 [SMILES: Cn1cc(-c2nc3cnccc3nc2N2CCN(Cc3ccc(F)cc3F)CC2=O)cn1] | 4-[(2,4- difluorophenyl)methyl]-1- [3-(1-methyl-1H-pyrazol- 4-yl)pyrido[3,4-b]pyrazin- 2-yl]piperazin-2-one | NA |
| 260 | 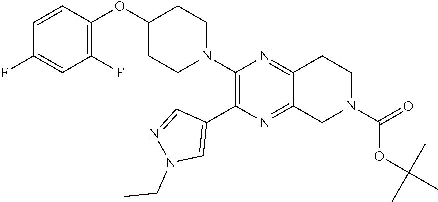 [SMILES: CCn1cc(-c2nc3c(nc2N2CCC(Oc4ccc(F)cc4F)CC2)CCN(C(=O)OC(C)(C)C)C3)cn1] | tert-butyl 2-[4-(2,4- difluorophenoxy)piperidin- 1-yl]-3-(1-ethyl-1H- pyrazol-4-yl)- 5H,6H,7H,8H-pyrido[3,4- b]pyrazine-6-carboxylate | NA |
| 261 | 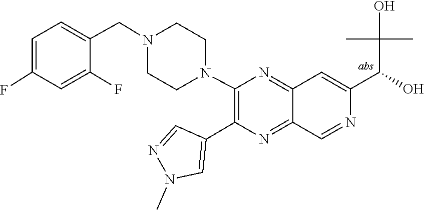 [SMILES: Cn1cc(-c2nc3cnc([C@@H](O)C(C)(C)O)cc3nc2N2CCN(Cc3ccc(F)cc3F)CC2)cn1] or enantiomer | (1R)-1-(2-{4-[(2,4- difluorophenyl)methyl] piperazin-1-yl}-3-(1-methyl- 1H-pyrazol-4- yl)pyrido[3,4-b]pyrazin-7- yl)-2-methylpropane-1,2- diol, or enantiomer | NA |
| 262 | 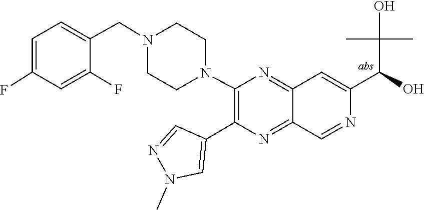 [SMILES: Cn1cc(-c2nc3cnc([C@H](O)C(C)(C)O)cc3nc2N2CCN(Cc3ccc(F)cc3F)CC2)cn1] or enantiomer | (1S)-1-(2-{4-[(2,4- difluorophenyl)methyl] piperazin-1-yl}-3-(1-methyl- 1H-pyrazol-4- yl)pyrido[3,4-b]pyrazin-7- yl)-2-methylpropane-1,2- diol, or enantiomer | NA |
| 263 | 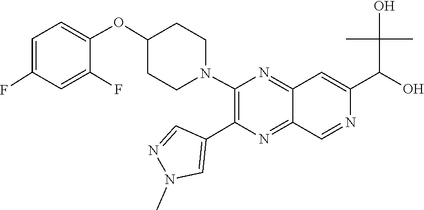 [SMILES: Cn1cc(-c2nc3cnc(C(O)C(C)(C)O)cc3nc2N2CCC(Oc3ccc(F)cc3F)CC2)cn1] | 1-{2-[4-(2,4- difluorophenoxy)piperidin- 1-yl]-3-(1-methyl-1H- pyrazol-4-yl)pyrido[3,4- b]pyrazin-7-yl}-2- methylpropane-1,2-diol | NA |
| 264 | 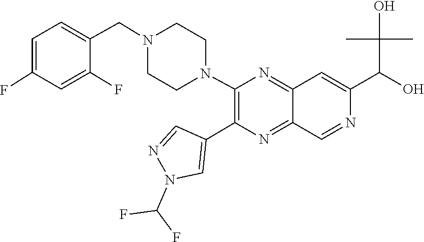 [SMILES: CC(C)(O)C(O)c1cc2nc(N3CCN(Cc4ccc(F)cc4F)CC3)c(-c3cnn(C(F)F)c3)nc2cn1] | 1-{3-[1-(difluoromethyl)- 1H-pyrazol-4-yl]-2-{4- [(2,4- difluorophenyl)methyl] piperazin-1-yl}pyrido[3,4- b]pyrazin-7-yl}-2- methylpropane-1,2-diol | NA |
| 265 | 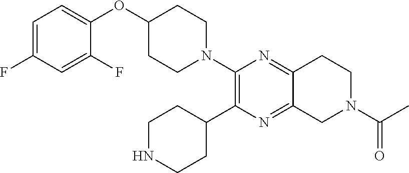 [SMILES: CC(=O)N1CCc2nc(N3CCC(Oc4ccc(F)cc4F)CC3)c(C3CCNCC3)nc2C1] | 1-{2-[4-(2,4- difluorophenoxy)piperidin- 1-yl]-3-(piperidin-4-yl)- 5H,6H,7H,8H-pyrido[3,4- b]pyrazin-6-yl}ethan-1- one | NA |
| 266 | 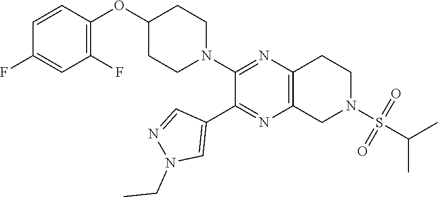 [SMILES: CCn1cc(-c2nc3c(nc2N2CCC(Oc4ccc(F)cc4F)CC2)CCN(S(=O)(=O)C(C)C)C3)cn1] | 4-(2,4-difluorophenoxy)- 1-[3-(1-ethyl-1H-pyrazol- 4-yl)-6-(propane-2- sulfonyl)-5H,6H,7H,8H- pyrido[3,4-b]pyrazin-2- yl]piperidine | NA |
| 267 | 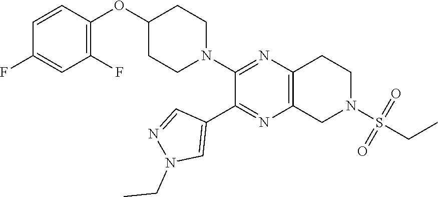 [SMILES: CCn1cc(-c2nc3c(nc2N2CCC(Oc4ccc(F)cc4F)CC2)CCN(S(=O)(=O)CC)C3)cn1] | 4-(2,4-difluorophenoxy)- 1-[6-(ethanesulfonyl)-3- (1-ethyl-1H-pyrazol-4-yl)- 5H,6H,7H,8H-pyrido[3,4- b]pyrazin-2-yl]piperidine | NA |
| 268 | 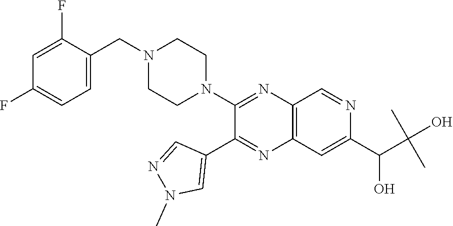 [SMILES: Cn1cc(-c2nc3cc(C(O)C(C)(C)O)ncc3nc2N2CCN(Cc3ccc(F)cc3F)CC2)cn1] | 1-(3-{4-[(2,4- difluorophenyl)methyl] piperazin-1-yl}-2-(1-methyl- 1H-pyrazol-4- yl)pyrido[3,4-b]pyrazin-7- yl)-2-methylpropane-1,2- diol | NA |
| 269 | 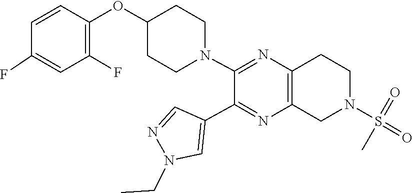 [SMILES: CCn1cc(-c2nc3c(nc2N2CCC(Oc4ccc(F)cc4F)CC2)CCN(S(C)(=O)=O)C3)cn1] | 4-(2,4-difluorophenoxy)- 1-[3-(1-ethyl-1H-pyrazol- 4-yl)-6-methanesulfonyl- 5H,6H,7H,8H-pyrido[3,4- b]pyrazin-2-yl]piperidine | NA |
| 270 | 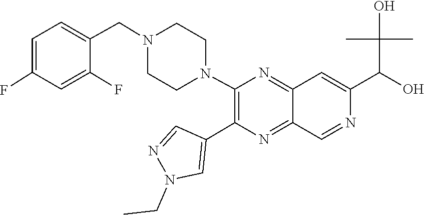 [SMILES: CCn1cc(-c2nc3cnc(C(O)C(C)(C)O)cc3nc2N2CCN(Cc3ccc(F)cc3F)CC2)cn1] | 1-(2-{4-[(2,4- difluorophenyl)methyl] piperazin-1-yl}-3-(1-ethyl- 1H-pyrazol-4- yl)pyrido[3,4-b]pyrazin-7- yl)-2-methylpropane-1,2- diol | NA |
| 271 | 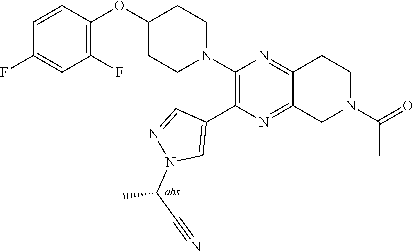 [SMILES: CC(=O)N1CCc2nc(N3CCC(Oc4ccc(F)cc4F)CC3)c(-c3cnn([C@@H](C)C#N)c3)nc2C1] or enantiomer | (2S)-2-(4-{6-acetyl-2-[4- (2,4- difluorophenoxy)piperidin- 1-yl]-5H,6H,7H,8H- pyrido[3,4-b]pyrazin-3- yl}-1H-pyrazol-1- yl)propanenitrile, or enantiomer | NA |
| 272 | 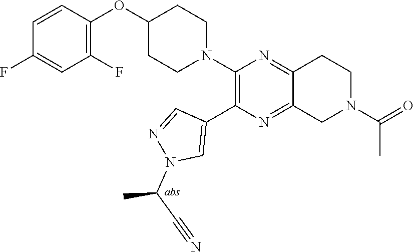 [SMILES: CC(=O)N1CCc2nc(N3CCC(Oc4ccc(F)cc4F)CC3)c(-c3cnn([C@H](C)C#N)c3)nc2C1] or enantiomer | (2R)-2-(4-{6-acetyl-2-[4- (2,4- difluorophenoxy)piperidin- 1-yl]-5H,6H,7H,8H- pyrido[3,4-b]pyrazin-3- yl}-1H-pyrazol-1- yl)propanenitrile, or enantiomer | NA |
| 273 | 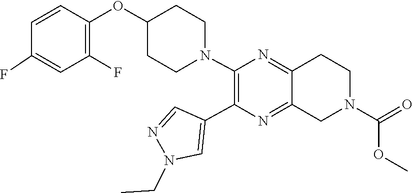 [SMILES: CCn1cc(-c2nc3c(nc2N2CCC(Oc4ccc(F)cc4F)CC2)CCN(C(=O)OC)C3)cn1] | methyl 2-[4-(2,4- difluorophenoxy)piperidin- 1-yl]-3-(1-ethyl-1H- pyrazol-4-yl)- 5H,6H,7H,8H-pyrido[3,4- b]pyrazine-6-carboxylate | NA |
| 274 | 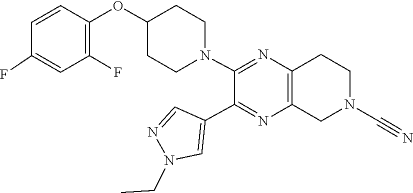 [SMILES: CCn1cc(-c2nc3c(nc2N2CCC(Oc4ccc(F)cc4F)CC2)CCN(C#N)C3)cn1] | 2-[4-(2,4- difluorophenoxy)piperidin- 1-yl]-3-(1-ethyl-1H- pyrazol-4-yl)- 5H,6H,7H,8H-pyrido[3,4- b]pyrazine-6-carbonitrile | NA |
| 275 | 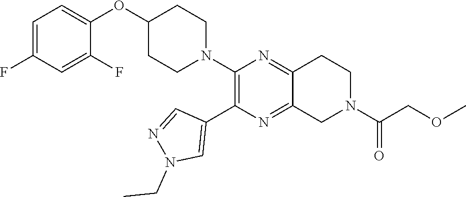 [SMILES: CCn1cc(-c2nc3c(nc2N2CCC(Oc4ccc(F)cc4F)CC2)CCN(C(=O)COC)C3)cn1] | 1-{2-[4-(2,4- difluorophenoxy)piperidin- 1-yl]-3-(1-ethyl-1H- pyrazol-4-yl)- 5H,6H,7H,8H-pyrido[3,4- b]pyrazin-6-yl}-2- methoxyethan-1-one | NA |
| 276 | 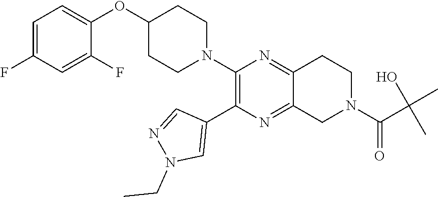 [SMILES: CCn1cc(-c2nc3c(nc2N2CCC(Oc4ccc(F)cc4F)CC2)CCN(C(=O)C(C)(C)O)C3)cn1] | 1-{2-[4-(2,4- difluorophenoxy)piperidin- 1-yl]-3-(1-ethyl-1H- pyrazol-4-yl)- 5H,6H,7H,8H-pyrido [3,4- b]pyrazin-6-yl}-2- hydroxy-2-methylpropan- 1-one | NA |
| 277 | 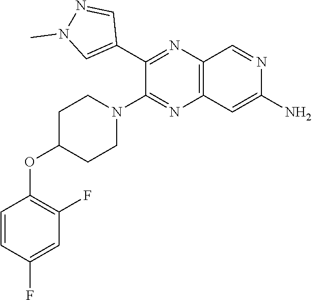 [SMILES: Cn1cc(-c2nc3cnc(N)cc3nc2N2CCC(Oc3ccc(F)cc3F)CC2)cn1] | 2-[4-(2,4- difluorophenoxy)piperidin- 1-yl]-3-(1-methyl-1H- pyrazol-4-yl)pyrido[3,4- b]pyrazin-7-amine | NA |
| 278 | 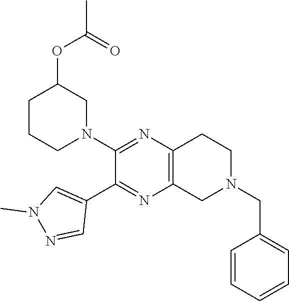 [SMILES: CC(=O)OC1CCCN(c2nc3c(nc2-c2cnn(C)c2)CN(Cc2ccccc2)CC3)C1] | 1-[6-benzyl-3-(1-methyl- 1H-pyrazol-4-yl)- 5H,6H,7H,8H-pyrido[3,4- b]pyrazin-2-yl]piperidin- 3-yl acetate | NA |
| 279 | 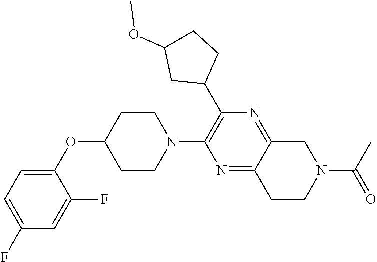 [SMILES: COC1CCC(c2nc3c(nc2N2CCC(Oc4ccc(F)cc4F)CC2)CCN(C(C)=O)C3)C1] | 1-{2-[4-(2,4- difluorophenoxy)piperidin- 1-yl]-3-(3- methoxycyclopentyl)- 5H,6H,7H,8H-pyrido[3,4- b]pyrazin-6-yl}ethan-1- one | NA |
| 280 | 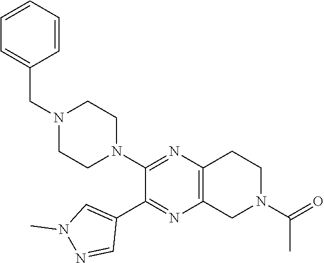 [SMILES: CC(=O)N1CCc2nc(N3CCN(Cc4ccccc4)CC3)c(-c3cnn(C)c3)nc2C1] | 1-[2-(4-benzylpiperazin-1- yl)-3-(1-methyl-1H- pyrazol-4-yl)- 5H,6H,7H,8H-pyrido[3,4- b]pyrazin-6-yl]ethan-1- one | NA |
| 281 | 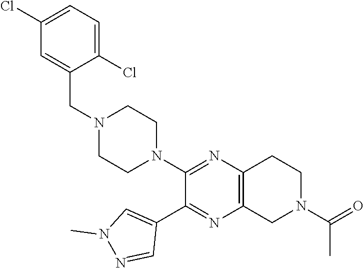 [SMILES: CC(=O)N1CCc2nc(N3CCN(Cc4cc(Cl)ccc4Cl)CC3)c(-c3cnn(C)c3)nc2C1] | 1-(2-{4-[(2,5- dichlorophenyl)methyl] piperazin-1-yl}-3-(1-methyl- 1H-pyrazol-4-yl)- 5H,6H,7H,8H-pyrido[3,4- b]pyrazin-6-yl)ethan-1- one | NA |
| 282 | 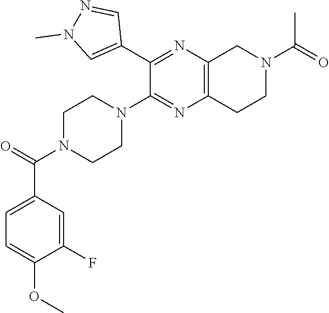 [SMILES: COc1ccc(C(=O)N2CCN(c3nc4c(nc3-c3cnn(C)c3)CN(C(C)=O)CC4)CC2)cc1F] | 1-{2-[4-(3-fluoro-4- methoxybenzoyl)piperazin- 1-yl]-3-(1-methyl-1H- pyrazol-4-yl)- 5H,6H,7H,8H-pyrido[3,4- b]pyrazin-6-yl}ethan-1- one | NA |
| 283 | 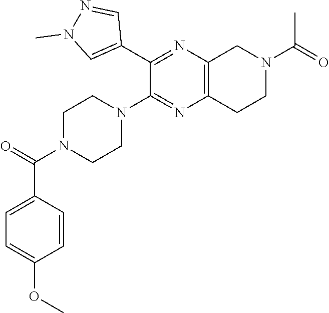 [SMILES: COc1ccc(C(=O)N2CCN(c3nc4c(nc3-c3cnn(C)c3)CN(C(C)=O)CC4)CC2)cc1] | 1-{2-[4-(4- methoxybenzoyl)piperazin- 1-yl]-3-(1-methyl-1H- pyrazol-4-yl)- 5H,6H,7H,8H-pyrido[3,4- b]pyrazin-6-yl}ethan-1- one | NA |
| 284 | 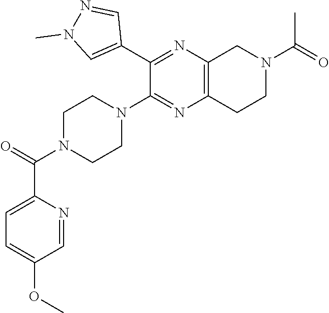 [SMILES: COc1ccc(C(=O)N2CCN(c3nc4c(nc3-c3cnn(C)c3)CN(C(C)=O)CC4)CC2)nc1] | 1-{2-[4-(5- methoxypyridine-2- carbonyl)piperazin-1-yl]- 3-(1-methyl-1H-pyrazol-4- yl)-5H,6H,7H,8H- pyrido[3,4-b]pyrazin-6- yl}ethan-1-one | NA |
| 285 | 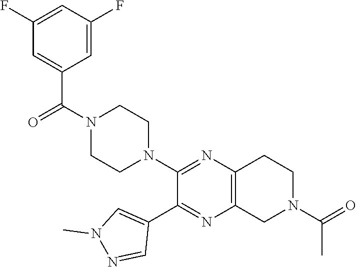 [SMILES: CC(=O)N1CCc2nc(N3CCN(C(=O)c4cc(F)cc(F)c4)CC3)c(-c3cnn(C)c3)nc2C1] | 1-{2-[4-(3,5- difluorobenzoyl)piperazin- 1-yl]-3-(1-methyl-1H- pyrazol-4-yl)- 5H,6H,7H,8H-pyrido[3,4- b]pyrazin-6-yl}ethan-1- one | NA |
| 286 | 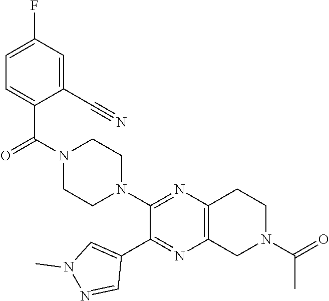 [SMILES: CC(=O)N1CCc2nc(N3CCN(C(=O)c4ccc(F)cc4C#N)CC3)c(-c3cnn(C)c3)nc2C1] | 2-{4-[6-acetyl-3-(1- methyl-1H-pyrazol-4-yl)- 5H,6H,7H,8H-pyrido[3,4- b]pyrazin-2-yl]piperazine- 1-carbonyl}-5- fluorobenzonitrile | NA |
| 287 | 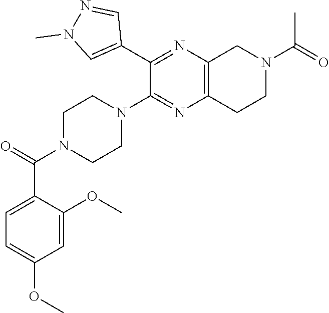 [SMILES: COc1ccc(C(=O)N2CCN(c3nc4c(nc3-c3cnn(C)c3)CN(C(C)=O)CC4)CC2)c(OC)c1] | 1-{2-[4-(2,4- dimethoxybenzoyl) piperazin-1-yl]-3- (1-methyl-1H- pyrazol-4-yl)- 5H,6H,7H,8H-pyrido[3,4- b]pyrazin-6-yl}ethan-1- one | NA |
| 288 | 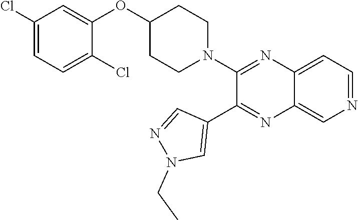 [SMILES: CCn1cc(-c2nc3cnccc3nc2N2CCC(Oc3cc(Cl)ccc3Cl)CC2)cn1] | 4-(2,5-dichlorophenoxy)- 1-[3-(1-ethyl-1H-pyrazol- 4-yl)pyrido[3,4-b]pyrazin- 2-yl]piperidine | NA |
| 289 | 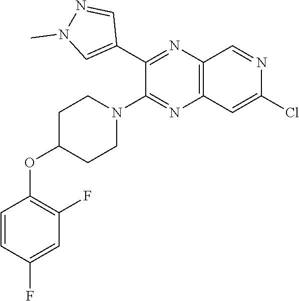 [SMILES: Cn1cc(-c2nc3cnc(Cl)cc3nc2N2CCC(Oc3ccc(F)cc3F)CC2)cn1] | 1-[7-chloro-3-(1-methyl- 1H-pyrazol-4- yl)pyrido[3,4-b]pyrazin-2- yl]-4-(2,4- difluorophenoxy)piperidine | NA |
| 290 | 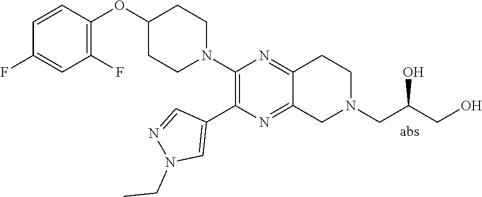 [SMILES: CCn1cc(-c2nc3c(nc2N2CCC(Oc4ccc(F)cc4F)CC2)CCN(C[C@@H](O)CO)C3)cn1] or enantiomer | (2R)-3-{2-[4-(2,4- difluorophenoxy)piperidin- 1-yl]-3-(1-ethyl-1H- pyrazol-4-yl)- 5H,6H,7H,8H-pyrido[3,4- b]pyrazin-6-yl}propane- 1,2-diol, or enantiomer | NA |
| 291 | 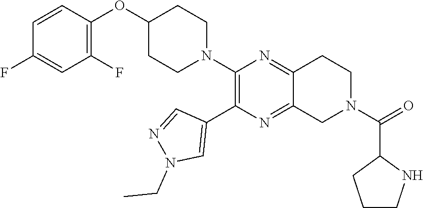 [SMILES: CCn1cc(-c2nc3c(nc2N2CCC(Oc4ccc(F)cc4F)CC2)CCN(C(=O)C2CCCN2)C3)cn1] | 4-(2,4-difluorophenoxy)- 1-[3-(1-ethyl-1H-pyrazol- 4-yl)-6-(pyrrolidine-2- carbonyl)-5H,6H,7H,8H- pyrido[3,4-b]pyrazin-2- yl]piperidine | NA |
| 292 | 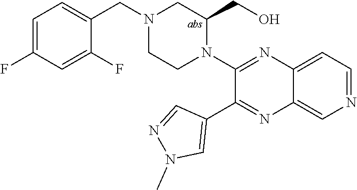 [SMILES: Cn1cc(-c2nc3cnccc3nc2N2CCN(Cc3ccc(F)cc3F)C[C@H]2CO)cn1] , or enantiomer | [(2S)-4-[(2,4- difluorophenyl)methyl]-1- [3-(1-methyl-1H-pyrazol- 4-yl)pyrido[3,4-b]pyrazin- 2-yl]piperazin-2- yl]methanol, or enantiomer | NA |
| 293 | 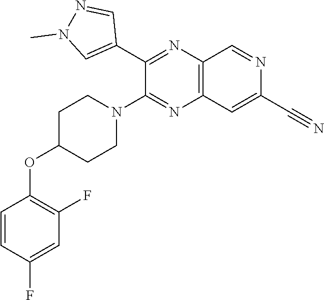 [SMILES: Cn1cc(-c2nc3cnc(C#N)cc3nc2N2CCC(Oc3ccc(F)cc3F)CC2)cn1] | 2-[4-(2,4- difluorophenoxy)piperidin- 1-yl]-3-(1-methyl-1H- pyrazol-4-yl)pyrido[3,4- b]pyrazine-7-carbonitrile | NA |
| 294 | 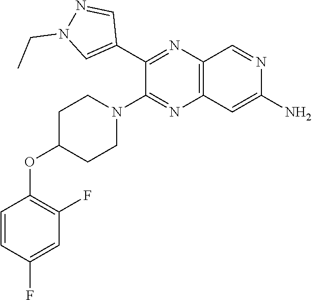 [SMILES: CCn1cc(-c2nc3cnc(N)cc3nc2N2CCC(Oc3ccc(F)cc3F)CC2)cn1] | 2-[4-(2,4- difluorophenoxy)piperidin- 1-yl]-3-(1-ethyl-1H- pyrazol-4-yl)pyrido[3,4- b]pyrazin-7-amine | NA |
| 295 | 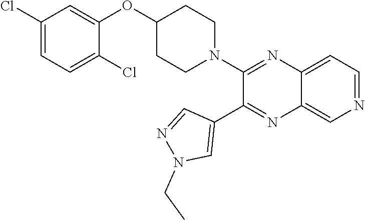 [SMILES: CCn1cc(-c2nc3cnccc3nc2N2CCC(Oc3cc(Cl)ccc3Cl)CC2)cn1] | 4-(5-chloro-2- fluorophenoxy)-1-[3-(1- ethyl-1H-pyrazol-4- yl)pyrido[3,4-b]pyrazin-2- yl]piperidine | NA |
| 296 | 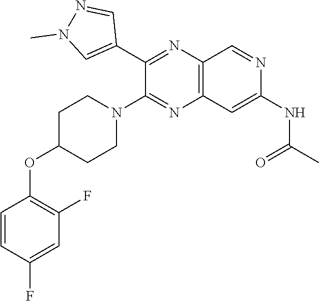 [SMILES: CC(=O)Nc1cc2nc(N3CCC(Oc4ccc(F)cc4F)CC3)c(-c3cnn(C)c3)nc2cn1] | N-{2-[4-(2,4- difluorophenoxy)piperidin- 1-yl]-3-(1-methyl-1H- pyrazol-4-yl)pyrido[3,4- b]pyrazin-7-yl}acetamide | NA |
| 297 | 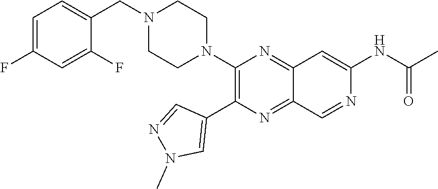 [SMILES: CC(=O)Nc1cc2nc(N3CCN(Cc4ccc(F)cc4F)CC3)c(-c3cnn(C)c3)nc2cn1] | N-(2-{4-[(2,4- difluorophenyl)methyl] piperazin-1-yl}-3-(1-methyl- 1H-pyrazol-4- yl)pyrido[3,4-b]pyrazin-7- yl)acetamide | NA |
| 298 | 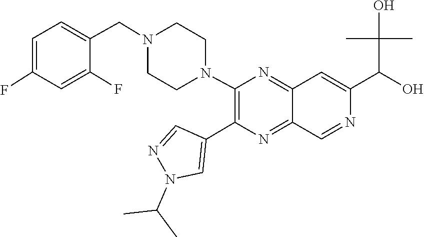 [SMILES: CC(C)n1cc(-c2nc3cnc(C(O)C(C)(C)O)cc3nc2N2CCN(Cc3ccc(F)cc3F)CC2)cn1] | 1-(2-{4-[(2,4- difluorophenyl)methyl] piperazin-1-yl}-3-[1-(propan- 2-yl)-1H-pyrazol-4- yl]pyrido[3,4-b]pyrazin-7- yl)-2-methylpropane-1,2- diol | NA |
| 299 | 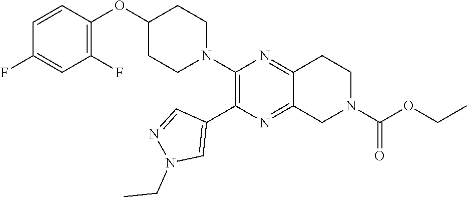 [SMILES: CCOC(=O)N1CCc2nc(N3CCC(Oc4ccc(F)cc4F)CC3)c(-c3cnn(CC)c3)nc2C1] | ethyl 2-[4-(2,4- difluorophenoxy)piperidin- 1-yl]-3-(1-ethyl-1H- pyrazol-4-yl)- 5H,6H,7H,8H-pyrido[3,4- b]pyrazine-6-carboxylate | NA |
| 300 | 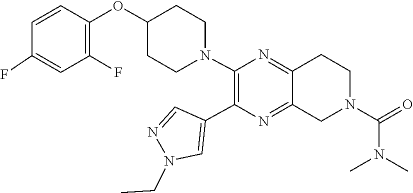 [SMILES: CCn1cc(-c2nc3c(nc2N2CCC(Oc4ccc(F)cc4F)CC2)CCN(C(=O)N(C)C)C3)cn1] | 2-[4-(2,4- difluorophenoxy)piperidin- 1-yl]-3-(1-ethyl-1H- pyrazol-4-yl)-N,N- dimethyl-5H,6H,7H,8H- pyrido[3,4-b]pyrazine-6- carboxamide | NA |
| 301 | 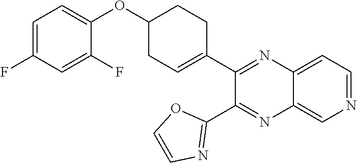 [SMILES: Fc1ccc(OC2CC=C(c3nc4ccncc4nc3-c3ncco3)CC2)c(F)c1] | 2-{2-[4-(2,4- difluorophenoxy)cyclohex- 1-en-1-yl]pyrido[3,4- b]pyrazin-3-yl}-1,3- oxazole | NA |
| 302 | 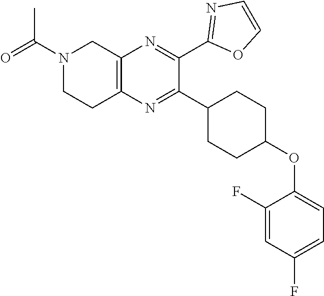 [SMILES: CC(=O)N1CCc2nc(C3CCC(Oc4ccc(F)cc4F)CC3)c(-c3ncco3)nc2C1] | 1-{2-[4-(2,4- difluorophenoxy)cyclohex yl]-3-(1,3-oxazol-2-yl)- 5H,6H,7H,8H-pyrido[3,4- b]pyrazin-6-yl}ethan-1- one | NA |
| 303 | 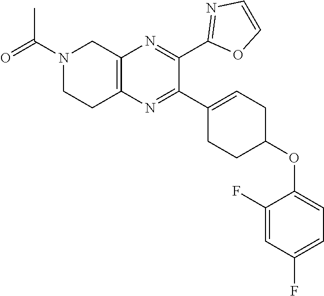 [SMILES: CC(=O)N1CCc2nc(C3=CCC(Oc4ccc(F)cc4F)CC3)c(-c3ncco3)nc2C1] | 1-{2-[4-(2,4- difluorophenoxy)cyclohex- 1-en-1-yl]-3-(1,3-oxazol- 2-yl)-5H,6H,7H,8H- pyrido[3,4-b]pyrazin-6- yl}ethan-1-one | NA |
| 304 | 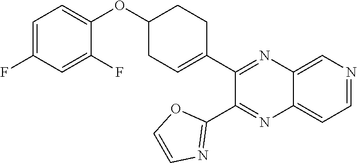 [SMILES: Fc1ccc(OC2CC=C(c3nc4cnccc4nc3-c3ncco3)CC2)c(F)c1] | 2-{3-[4-(2,4- difluorophenoxy)cyclohex- 1-en-1-yl]pyrido[3,4- b]pyrazin-2-yl}-1,3- oxazole | NA |
| 305 | 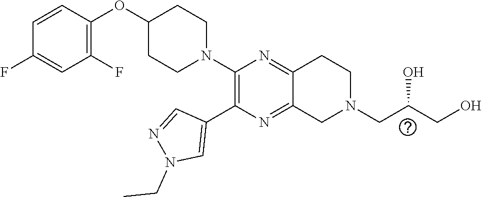 [SMILES: CCn1cc(-c2nc3c(nc2N2CCC(Oc4ccc(F)cc4F)CC2)CCN(C[C@H](O)CO)C3)cn1] | (2S)-3-{2-[4-(2,4- difluorophenoxy)piperidin- 1-yl]-3-(1-ethyl-1H- pyrazol-4-yl)- 5H,6H,7H,8H-pyrido[3,4- b]pyrazin-6-yl}propane- 1,2-diol | NA |
| 306 | 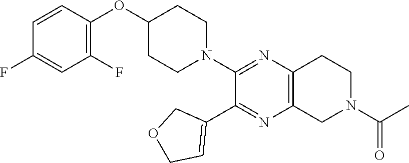 [SMILES: CC(=O)N1CCc2nc(N3CCC(Oc4ccc(F)cc4F)CC3)c(C3=CCOC3)nc2C1] | 1-{2-[4-(2,4- difluorophenoxy)piperidin- 1-yl]-3-(2,5- dihydrofuran-3-yl)- 5H,6H,7H,8H-pyrido[3,4- b]pyrazin-6-yl}ethan-1- one | NA |
| 307 | 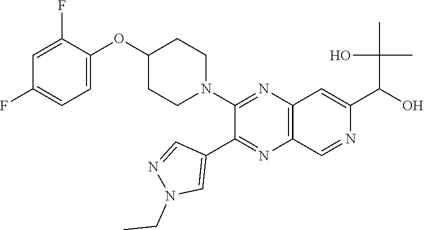 [SMILES: CCn1cc(-c2nc3cnc(C(O)C(C)(C)O)cc3nc2N2CCC(Oc3ccc(F)cc3F)CC2)cn1] | 1-{2-[4-(2,4- difluorophenoxy)piperidin- 1-yl]-3-(1-ethyl-1H- pyrazol-4-yl)pyrido[3,4- b]pyrazin-7-yl}-2- methylpropane-1,2-diol | NA |
| 308 | 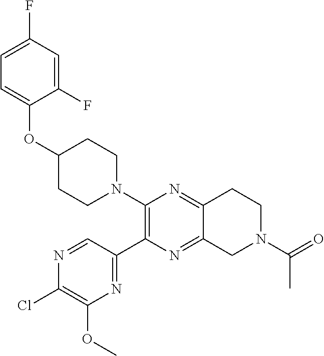 [SMILES: COc1nc(-c2nc3c(nc2N2CCC(Oc4ccc(F)cc4F)CC2)CCN(C(C)=O)C3)cnc1Cl] | 1-[3-(5-chloro-6- methoxypyrazin-2-yl)-2- [4-(2,4- difluorophenoxy)piperidin- 1-yl]-5H,6H,7H,8H- pyrido[3,4-b]pyrazin-6- yllethan-1-one | NA |
| 309 | 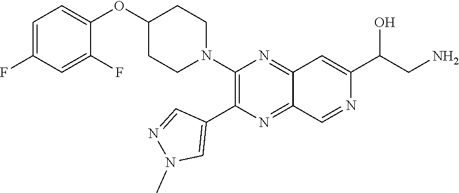 [SMILES: Cn1cc(-c2nc3cnc(C(O)CN)cc3nc2N2CCC(Oc3ccc(F)cc3F)CC2)cn1] | 2-amino-1-{2-[4-(2,4- difluorophenoxy)piperidin- 1-yl]-3-(1-methyl-1H- pyrazol-4-yl)pyrido[3,4- b]pyrazin-7-yl}ethan-1-ol | NA |
| 310 | 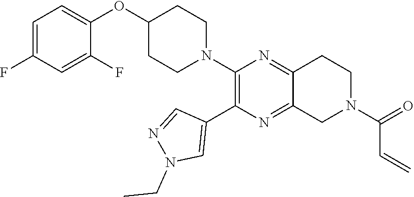 [SMILES: C=CC(=O)N1CCc2nc(N3CCC(Oc4ccc(F)cc4F)CC3)c(-c3cnn(CC)c3)nc2C1] | 1-{2-[4-(2,4- difluorophenoxy)piperidin- 1-yl]-3-(1-ethyl-1H- pyrazol-4-yl)- 5H,6H,7H,8H-pyrido[3,4- b]pyrazin-6-yl}prop-2-en- 1-one | NA |
| 311 | 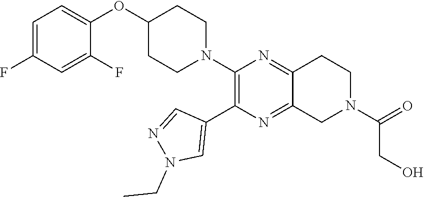 [SMILES: CCn1cc(-c2nc3c(nc2N2CCC(Oc4ccc(F)cc4F)CC2)CCN(C(=O)CO)C3)cn1] | 1-{2-[4-(2,4- difluorophenoxy)piperidin- 1-yl]-3-(1-ethyl-1H- pyrazol-4-yl)- 5H,6H,7H,8H-pyrido[3,4- b]pyrazin-6-yl}-2- hydroxyethan-1-one | NA |
| 312 | 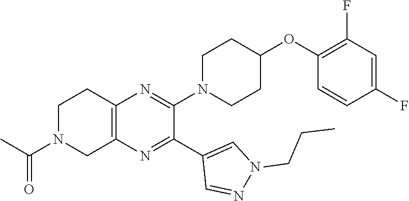 [SMILES: CCCn1cc(-c2nc3c(nc2N2CCC(Oc4ccc(F)cc4F)CC2)CCN(C(C)=O)C3)cn1] | 1-{2-[4-(2,4- difluorophenoxy)piperidin- 1-yl]-3-(1-propyl-1H- pyrazol-4-yl)- 5H,6H,7H,8H-pyrido[3,4- b]pyrazin-6-yl}ethan-1- one | NA |
| 313 | 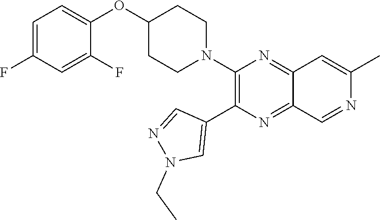 [SMILES: CCn1cc(-c2nc3cnc(C)cc3nc2N2CCC(Oc3ccc(F)cc3F)CC2)cn1] | 4-(2,4-difluorophenoxy)- 1-[3-(1-ethyl-1H-pyrazol- 4-yl)-7-methylpyrido[3,4- b]pyrazin-2-yl]piperidine | NA |
| 314 | 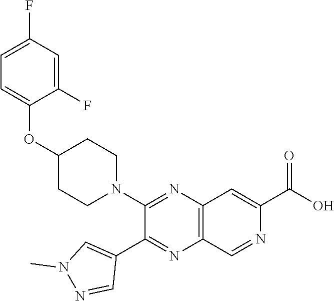 [SMILES: Cn1cc(-c2nc3cnc(C(=O)O)cc3nc2N2CCC(Oc3ccc(F)cc3F)CC2)cn1] | 2-[4-(2,4- difluorophenoxy)piperidin- 1-yl]-3-(1-methyl-1H- pyrazol-4-yl)pyrido[3,4- b]pyrazine-7-carboxylic acid | NA |
| 315 | 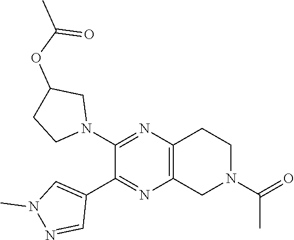 [SMILES: CC(=O)OC1CCN(c2nc3c(nc2-c2cnn(C)c2)CN(C(C)=O)CC3)C1] | 1-[6-acetyl-3-(1-methyl- 1H-pyrazol-4-yl)- 5H,6H,7H,8H-pyrido[3,4- b]pyrazin-2-yl]pyrrolidin- 3-yl acetate | NA |
| 316 | 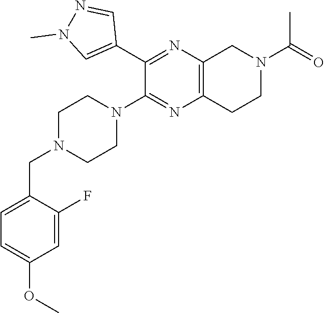 [SMILES: COc1ccc(CN2CCN(c3nc4c(nc3-c3cnn(C)c3)CN(C(C)=O)CC4)CC2)c(F)c1] | 1-(2-{4-[(2-fluoro-4- methoxyphenyl)methyl] piperazin-1-yl}-3-(1-methyl- 1H-pyrazol-4-yl)- 5H,6H,7H,8H-pyrido[3,4- b]pyrazin-6-yl)ethan-1- one | NA |
| 317 | 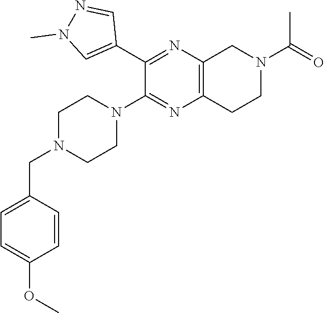 [SMILES: COc1ccc(CN2CCN(c3nc4c(nc3-c3cnn(C)c3)CN(C(C)=O)CC4)CC2)cc1] | 1-(2-{4-[(4- methoxyphenyl)methyl] piperazin-1-yl}-3-(1-methyl- 1H-pyrazol-4-yl)- 5H,6H,7H,8H-pyrido[3,4- b]pyrazin-6-yl)ethan-1- one | NA |
| 318 | 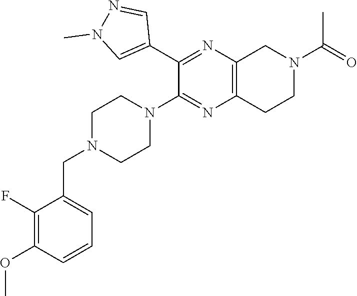 [SMILES: COc1cccc(CN2CCN(c3nc4c(nc3-c3cnn(C)c3)CN(C(C)=O)CC4)CC2)c1F] | 1-(2-{4-[(2-fluoro-3- methoxyphenyl)methyl] piperazin-1-yl}-3-(1-methyl- 1H-pyrazol-4-yl)- 5H,6H,7H,8H-pyrido[3,4- b]pyrazin-6-yl)ethan-1- one | NA |
| 319 | 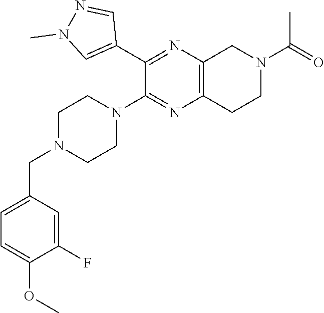 [SMILES: COc1ccc(CN2CCN(c3nc4c(nc3-c3cnn(C)c3)CN(C(C)=O)CC4)CC2)cc1F] | 1-(2-{4-[(3-fluoro-4- methoxyphenyl)methyl] piperazin-1-yl}-3-(1-methyl- 1H-pyrazol-4-yl)- 5H,6H,7H,8H-pyrido[3,4- b]pyrazin-6-yl)ethan-1- one | NA |
| 320 | 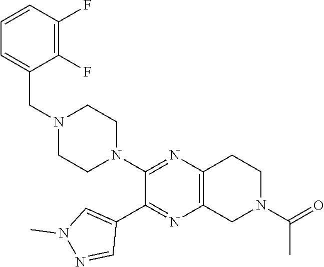 [SMILES: CC(=O)N1CCc2nc(N3CCN(Cc4cccc(F)c4F)CC3)c(-c3cnn(C)c3)nc2C1] | 1-(2-{4-[(2,3- difluorophenyl)methyl] piperazin-1-yl}-3-(1-methyl- 1H-pyrazol-4-yl)- 5H,6H,7H,8H-pyrido[3,4- b]pyrazin-6-yl)ethan-1- one | NA |
| 321 | 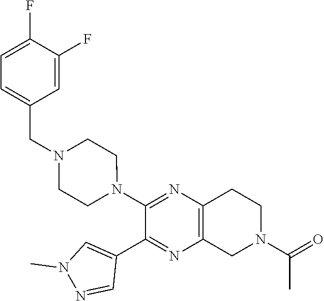 [SMILES: CC(=O)N1CCc2nc(N3CCN(Cc4ccc(F)c(F)c4)CC3)c(-c3cnn(C)c3)nc2C1] | 1-(2-{4-[(3,4- difluorophenyl)methyl] piperazin-1-yl}-3-(1-methyl- 1H-pyrazol-4-yl)- 5H,6H,7H,8H-pyrido[3,4- b]pyrazin-6-yl)ethan-1- one | NA |
| 322 | 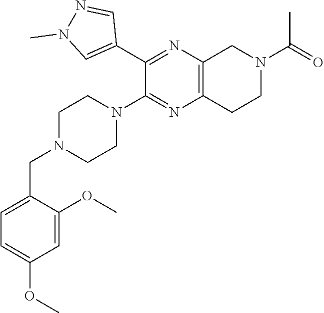 [SMILES: COc1ccc(CN2CCN(c3nc4c(nc3-c3cnn(C)c3)CN(C(C)=O)CC4)CC2)c(OC)c1] | 1-(2-{4-[(2,4- dimethoxyphenyl)methyl] piperazin-1-yl}-3-(1- methyl-1H-pyrazol-4-yl)- 5H,6H,7H,8H-pyrido[3,4- b]pyrazin-6-yl)ethan-1- one | NA |
| 323 | 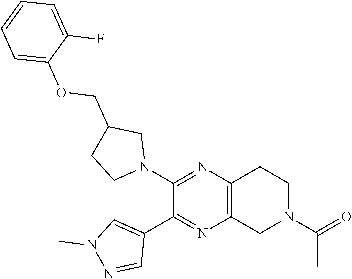 [SMILES: CC(=O)N1CCc2nc(N3CCC(COc4ccccc4F)C3)c(-c3cnn(C)c3)nc2C1] | 1-(2-{3-[(2- fluorophenoxy)methyl] pyrrolidin-1-yl}-3-(1-methyl- 1H-pyrazol-4-yl)- 5H,6H,7H,8H-pyrido[3,4- b]pyrazin-6-yl)ethan-1- one | NA |
| 324 | 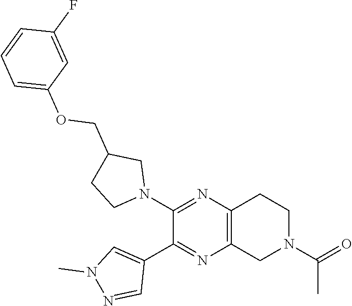 [SMILES: CC(=O)N1CCc2nc(N3CCC(COc4cccc(F)c4)C3)c(-c3cnn(C)c3)nc2C1] | 1-(2-{3-[(3- fluorophenoxy)methyl] pyrrolidin-1-yl}-3-(1-methyl- 1H-pyrazol-4-yl)- 5H,6H,7H,8H-pyrido[3,4- b]pyrazin-6-yl)ethan-1- one | NA |
| 325 | 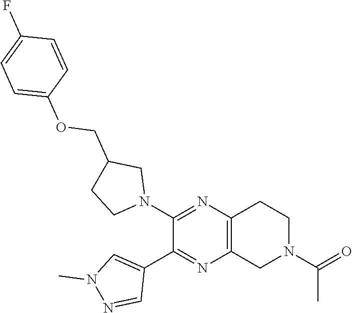 [SMILES: CC(=O)N1CCc2nc(N3CCC(COc4ccc(F)cc4)C3)c(-c3cnn(C)c3)nc2C1] | 1-(2-{3-[(4- fluorophenoxy)methyl] pyrrolidin-1-yl}-3-(1-methyl- 1H-pyrazol-4-yl)- 5H,6H,7H,8H-pyrido[3,4- b]pyrazin-6-yl)ethan-1- one | NA |
| 326 | 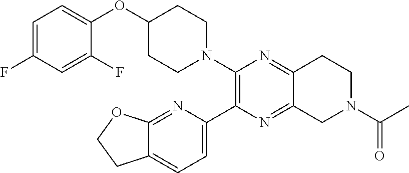 [SMILES: CC(=O)N1CCc2nc(N3CCC(Oc4ccc(F)cc4F)CC3)c(-c3ccc4c(n3)OCC4)nc2C1] | 1-{2-[4-(2,4- difluorophenoxy)piperidin- 1-yl]-3-{2H,3H-furo[2,3- b]pyridin-6-yl}- 5H,6H,7H,8H-pyrido[3,4- b]pyrazin-6-yl}ethan-1- one | NA |
| 327 | 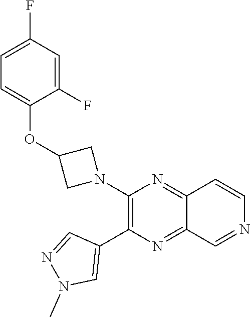 [SMILES: Cn1cc(-c2nc3cnccc3nc2N2CC(Oc3ccc(F)cc3F)C2)cn1] | 4-{2-[3-(2,4- difluorophenoxy)azetidin- 1-yl]pyrido[3,4-b]pyrazin- 3-yl}-1-methyl-1H- pyrazole | NA |
| 328 | 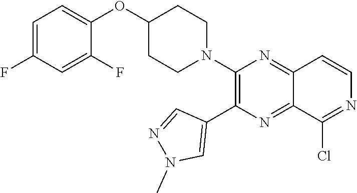 [SMILES: Cn1cc(-c2nc3c(Cl)nccc3nc2N2CCC(Oc3ccc(F)cc3F)CC2)cn1] | 1-[5-chloro-3-(1-methyl- 1H-pyrazol-4- yl)pyrido[3,4-b]pyrazin-2- yl]-4-(2,4- difluorophenoxy)piperidine | NA |
| 329 | 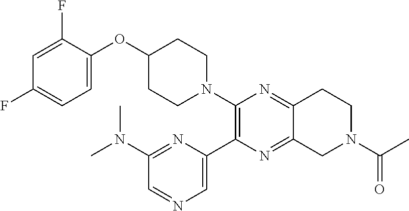 [SMILES: CC(=O)N1CCc2nc(N3CCC(Oc4ccc(F)cc4F)CC3)c(-c3cncc(N(C)C)n3)nc2C1] | 1-{2-[4-(2,4- difluorophenoxy)piperidin- 1-yl]-3-[6- (methylamino)pyrazin-2- yl]-5H,6H,7H,8H- pyrido[3,4-b]pyrazin-6- yl}ethan-1-one | NA |
| 330 | 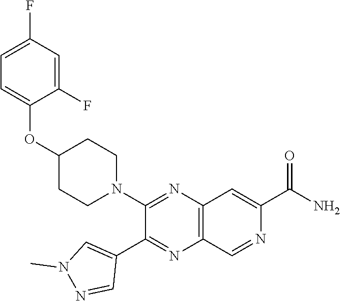 [SMILES: Cn1cc(-c2nc3cnc(C(N)=O)cc3nc2N2CCC(Oc3ccc(F)cc3F)CC2)cn1] | 2-[4-(2,4- difluorophenoxy)piperidin- 1-yl]-3-(1-methyl-1H- pyrazol-4-yl)pyrido[3,4- b]pyrazine-7-carboxamide | NA |
| 331 | 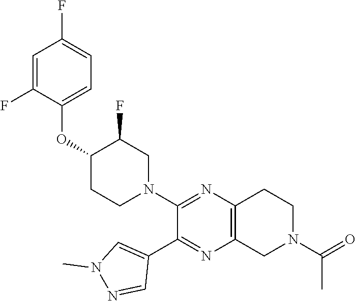 [SMILES: CC(=O)N1CCc2nc(N3CC[C@H](Oc4ccc(F)cc4F)[C@@H](F)C3)c(-c3cnn(C)c3)nc2C1] or enantiomer | 1-{2-[(3S,4S)-4-(2,4- difluorophenoxy)-3- fluoropiperidin-1-yl]-3-(1- methyl-1H-pyrazol-4-yl)- 5H,6H,7H,8H-pyrido[3,4- b]pyrazin-6-yl}ethan-1- one, or enantiomer | NA |
| 332 | 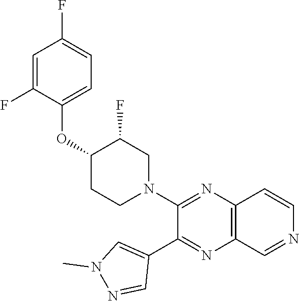 [SMILES: Cn1cc(-c2nc3cnccc3nc2N2CC[C@H](Oc3ccc(F)cc3F)[C@H](F)C2)cn1] or enantiomer | (3R,4S)-4-(2,4- difluorophenoxy)-3- fluoro-1-[3-(1-methyl-1H- pyrazol-4-yl)pyrido[3,4- b]pyrazin-2-yl]piperidine, or enantiomer | NA |
| 333 | 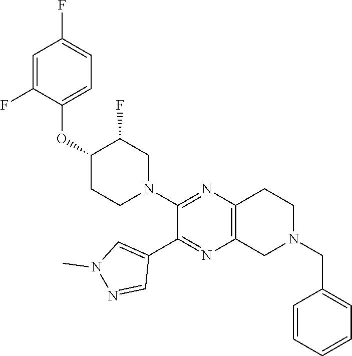 [SMILES: Cn1cc(-c2nc3c(nc2N2CC[C@H](Oc4ccc(F)cc4F)[C@H](F)C2)CCN(Cc2ccccc2)C3)cn1] , or enantiomer | (3R,4S)-1-[6-benzyl-3-(1- methyl-1H-pyrazol-4-yl)- 5H,6H,7H,8H-pyrido[3,4- b]pyrazin-2-yl]-4-(2,4- difluorophenoxy)-3- fluoropiperidine, or enantiomer | NA |
| 334 | 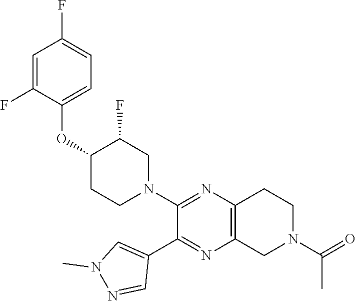 [SMILES: CC(=O)N1CCc2nc(N3CC[C@H](Oc4ccc(F)cc4F)[C@H](F)C3)c(-c3cnn(C)c3)nc2C1] or enantiomer | 1-{2-[(3R,4S)-4-(2,4- difluorophenoxy)-3- fluoropiperidin-1-yl]-3-(1- methyl-1H-pyrazol-4-yl)- 5H,6H,7H,8H-pyrido[3,4- b]pyrazin-6-yl}ethan-1- one, or enantiomer | NA |
| 335 | 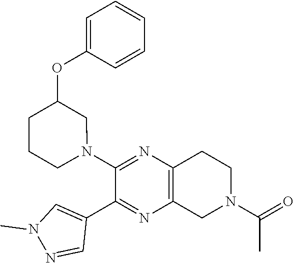 [SMILES: CC(=O)N1CCc2nc(N3CCCC(Oc4ccccc4)C3)c(-c3cnn(C)c3)nc2C1] | 1-[3-(1-methyl-1H- pyrazol-4-yl)-2-(3- phenoxypiperidin-1-yl)- 5H,6H,7H,8H-pyrido[3,4- b]pyrazin-6-yl]ethan-1- one | NA |
| 336 | 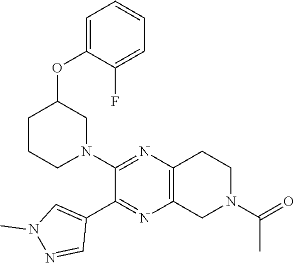 [SMILES: CC(=O)N1CCc2nc(N3CCCC(Oc4ccccc4F)C3)c(-c3cnn(C)c3)nc2C1] | 1-{2-[3-(2- fluorophenoxy)piperidin- 1-yl]-3-(1-methyl-1H- pyrazol-4-yl)- 5H,6H,7H,8H-pyrido[3,4- b]pyrazin-6-yl}ethan-1- one | NA |
| 337 | 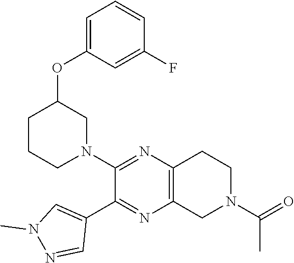 [SMILES: CC(=O)N1CCc2nc(N3CCCC(Oc4cccc(F)c4)C3)c(-c3cnn(C)c3)nc2C1] | 1-{2-[3-(3- fluorophenoxy)piperidin- 1-yl]-3-(1-methyl-1H- pyrazol-4-yl)- 5H,6H,7H,8H-pyrido[3,4- b]pyrazin-6-yl}ethan-1- one | NA |
| 338 | 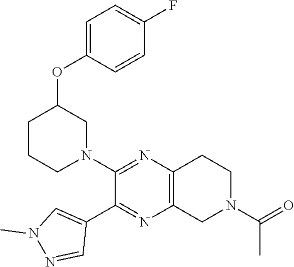 [SMILES: CC(=O)N1CCc2nc(N3CCCC(Oc4ccc(F)cc4)C3)c(-c3cnn(C)c3)nc2C1] | 1-{2-[3-(4- fluorophenoxy)piperidin- 1-yl]-3-(1-methyl-1H- pyrazol-4-yl)- 5H,6H,7H,8H-pyrido[3,4- b]pyrazin-6-yl}ethan-1- one | NA |
| 339 | 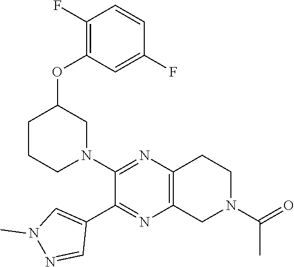 [SMILES: CC(=O)N1CCc2nc(N3CCCC(Oc4cc(F)ccc4F)C3)c(-c3cnn(C)c3)nc2C1] | 1-{2-[3-(2,5- difluorophenoxy)piperidin- 1-yl]-3-(1-methyl-1H- pyrazol-4-yl)- 5H,6H,7H,8H-pyrido[3,4- b]pyrazin-6-yl}ethan-1- one | NA |
| 340 | 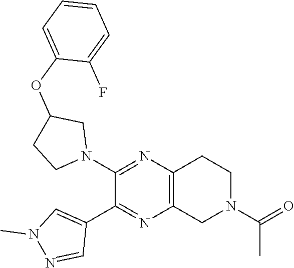 [SMILES: CC(=O)N1CCc2nc(N3CCC(Oc4ccccc4F)C3)c(-c3cnn(C)c3)nc2C1] | 1-{2-[3-(2- fluorophenoxy)pyrrolidin- 1-yl]-3-(1-methyl-1H- pyrazol-4-yl)- 5H,6H,7H,8H-pyrido[3,4- b]pyrazin-6-yl}ethan-1- one | NA |
| 341 | 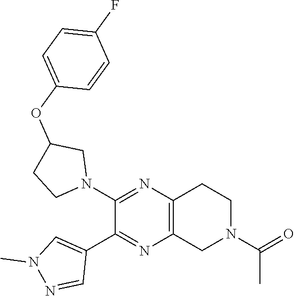 [SMILES: CC(=O)N1CCc2nc(N3CCC(Oc4ccc(F)cc4)C3)c(-c3cnn(C)c3)nc2C1] | 1-{2-[3-(4- fluorophenoxy)pyrrolidin- 1-yl]-3-(1-methyl-1H- pyrazol-4-yl)- 5H,6H,7H,8H-pyrido[3,4- b]pyrazin-6-yl}ethan-1- one | NA |
| 342 | 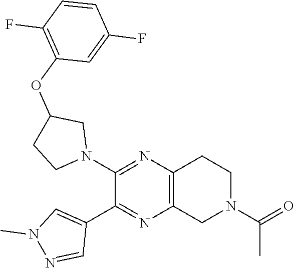 [SMILES: CC(=O)N1CCc2nc(N3CCC(Oc4cc(F)ccc4F)C3)c(-c3cnn(C)c3)nc2C1] | 1-{2-[3-(2,5- difluorophenoxy)pyrrolidin- 1-yl]-3-(1-methyl-1H- pyrazol-4-yl)- 5H,6H,7H,8H-pyrido[3,4- b]pyrazin-6-yl}ethan-1- one | NA |
| 343 | 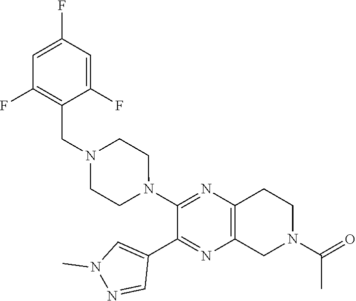 [SMILES: CC(=O)N1CCc2nc(N3CCN(Cc4c(F)cc(F)cc4F)CC3)c(-c3cnn(C)c3)nc2C1] | 1-[3-(1-methyl-1H- pyrazol-4-yl)-2-{4-[(2,4,6- trifluorophenyl)methyl] piperazin-1-yl}- 5H,6H,7H,8H-pyrido[3,4- b]pyrazin-6-yl]ethan-1- one | NA |
| 344 | 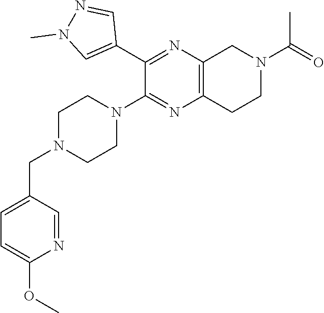 [SMILES: COc1ccc(CN2CCN(c3nc4c(nc3-c3cnn(C)c3)CN(C(C)=O)CC4)CC2)cn1] | 1-(2-{4-[(5- methoxypyridin-2- yl)methyl]piperazin-1-yl}- 3-(1-methyl-1H-pyrazol-4- yl)-5H,6H,7H,8H- pyrido[3,4-b]pyrazin-6- yl)ethan-1-one | NA |
| 345 | 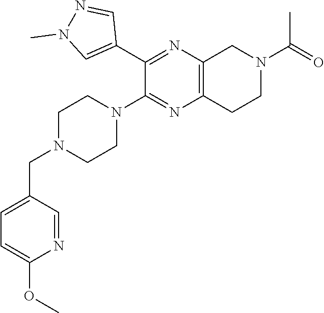 [SMILES: COc1ccc(CN2CCN(c3nc4c(nc3-c3cnn(C)c3)CN(C(C)=O)CC4)CC2)cn1] | 1-(2-{4-[(6- methoxypyridin-3- yl)methyl]piperazin-1-yl}- 3-(1-methyl-1H-pyrazol-4- yl)-5H,6H,7H,8H- pyrido[3,4-b]pyrazin-6- yl)ethan-1-one | NA |
| 346 | 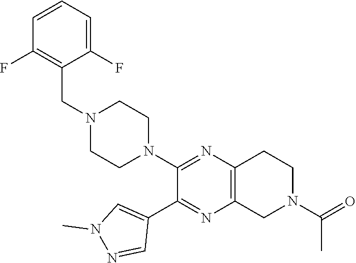 [SMILES: CC(=O)N1CCc2nc(N3CCN(Cc4c(F)cccc4F)CC3)c(-c3cnn(C)c3)nc2C1] | 1-(2-{4-[(2,6- difluorophenyl)methyl] piperazin-1-yl}-3-(1-methyl- 1H-pyrazol-4-yl)- 5H,6H,7H,8H-pyrido[3,4- b]pyrazin-6-yl)ethan-1- one | NA |
| 347 | 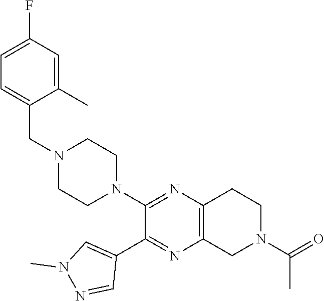 [SMILES: CC(=O)N1CCc2nc(N3CCN(Cc4ccc(F)cc4C)CC3)c(-c3cnn(C)c3)nc2C1] | 1-(2-{4-[(4-fluoro-2- methylphenyl)methyl] piperazin-1-yl}-3-(1-methyl- 1H-pyrazol-4-yl)- 5H,6H,7H,8H-pyrido[3,4- b]pyrazin-6-yl)ethan-1- one | NA |
| 348 | 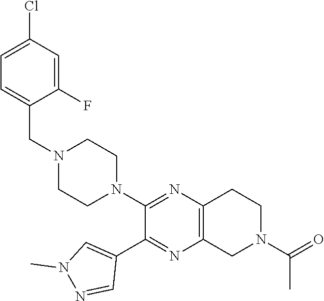 [SMILES: CC(=O)N1CCc2nc(N3CCN(Cc4ccc(Cl)cc4F)CC3)c(-c3cnn(C)c3)nc2C1] | 1-(2-{4-[(4-chloro-2- fluorophenyl)methyl] piperazin-1-yl}-3-(1-methyl- 1H-pyrazol-4-yl)- 5H,6H,7H,8H-pyrido[3,4- b]pyrazin-6-yl)ethan-1- one | NA |
| 349 | 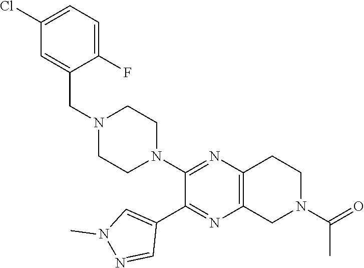 [SMILES: CC(=O)N1CCc2nc(N3CCN(Cc4cc(Cl)ccc4F)CC3)c(-c3cnn(C)c3)nc2C1] | 1-(2-{4-[(5-chloro-2- fluorophenyl)methyl] piperazin-1-yl}-3-(1-methyl- 1H-pyrazol-4-yl)- 5H,6H,7H,8H-pyrido[3,4- b]pyrazin-6-yl)ethan-1- one | NA |
| 350 | 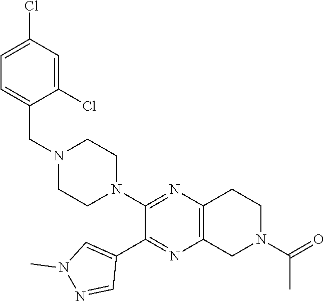 [SMILES: CC(=O)N1CCc2nc(N3CCN(Cc4ccc(Cl)cc4Cl)CC3)c(-c3cnn(C)c3)nc2C1] | 1-(2-{4-[(2,4- dichlorophenyl)methyl] piperazin-1-yl}-3-(1-methyl- 1H-pyrazol-4-yl)- 5H,6H,7H,8H-pyrido[3,4- b]pyrazin-6-yl)ethan-1- one | NA |
| 351 | 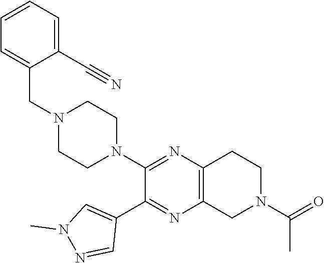 [SMILES: CC(=O)N1CCc2nc(N3CCN(Cc4ccccc4C#N)CC3)c(-c3cnn(C)c3)nc2C1] | 2-({4-[6-acetyl-3-(1- methyl-1H-pyrazol-4-yl)- 5H,6H,7H,8H-pyrido[3,4- b]pyrazin-2-yl]piperazin- 1-yl}methyl)benzonitrile | NA |
| 352 | 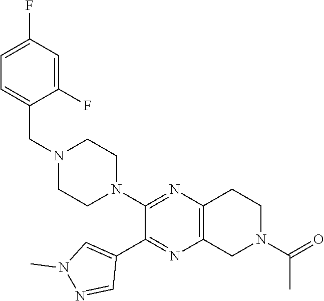 [SMILES: CC(=O)N1CCc2nc(N3CCN(Cc4ccc(F)cc4F)CC3)c(-c3cnn(C)c3)nc2C1] | 1-(2-{4-[(2,4- difluorophenyl)methyl] piperazin-1-yl}-3-(1-methyl- 1H-pyrazol-4-yl)- 5H,6H,7H,8H-pyrido[3,4- b]pyrazin-6-yl)ethan-1- one | NA |
| 353 | 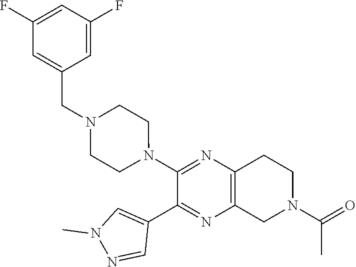 [SMILES: CC(=O)N1CCc2nc(N3CCN(Cc4cc(F)cc(F)c4)CC3)c(-c3cnn(C)c3)nc2C1] | 1-(2-{4-[(3,5- difluorophenyl)methyl] piperazin-1-yl}-3-(1-methyl- 1H-pyrazol-4-yl)- 5H,6H,7H,8H-pyrido[3,4- b]pyrazin-6-yl)ethan-1- one | NA |
| 354 | 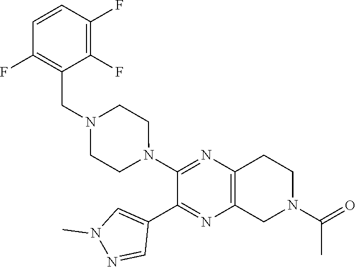 [SMILES: CC(=O)N1CCc2nc(N3CCN(Cc4c(F)ccc(F)c4F)CC3)c(-c3cnn(C)c3)nc2C1] | 1-[3-(1-methyl-1H- pyrazol-4-yl)-2-{4-[(2,3,6- trifluorophenyl)methyl] piperazin-1-yl}- 5H,6H,7H,8H-pyrido[3,4- b]pyrazin-6-yl]ethan-1- one | NA |
| 355 | 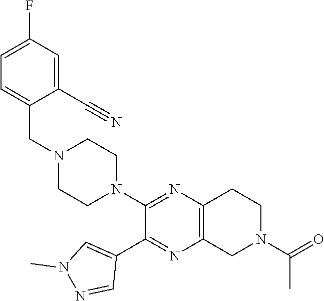 [SMILES: CC(=O)N1CCc2nc(N3CCN(Cc4ccc(F)cc4C#N)CC3)c(-c3cnn(C)c3)nc2C1] | 2-({4-[6-acetyl-3-(1- methyl-1H-pyrazol-4-yl)- 5H,6H,7H,8H-pyrido[3,4- b]pyrazin-2-yl]piperazin- 1-yl}methyl)-5- fluorobenzonitrile | NA |
| 356 | 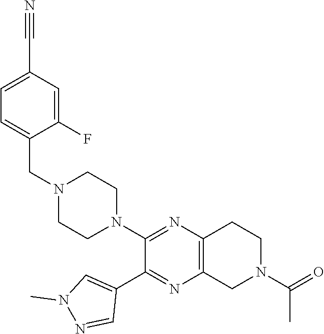 [SMILES: CC(=O)N1CCc2nc(N3CCN(Cc4ccc(C#N)cc4F)CC3)c(-c3cnn(C)c3)nc2C1] | 4-({4-[6-acetyl-3-(1- methyl-1H-pyrazol-4-yl)- 5H,6H,7H,8H-pyrido[3,4- b]pyrazin-2-yl]piperazin- 1-yl}methyl)-3- fluorobenzonitrile | NA |
| 357 | 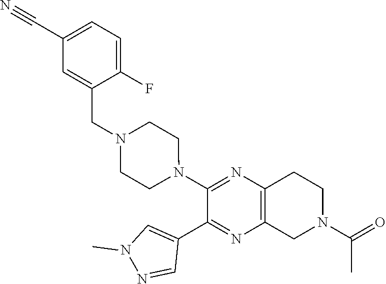 [SMILES: CC(=O)N1CCc2nc(N3CCN(Cc4cc(C#N)ccc4F)CC3)c(-c3cnn(C)c3)nc2C1] | 3-({4-[6-acetyl-3-(1- methyl-1H-pyrazol-4-yl)- 5H,6H,7H,8H-pyrido[3,4- b]pyrazin-2-yl]piperazin- 1-yl}methyl)-4- fluorobenzonitrile | NA |
| 358 | 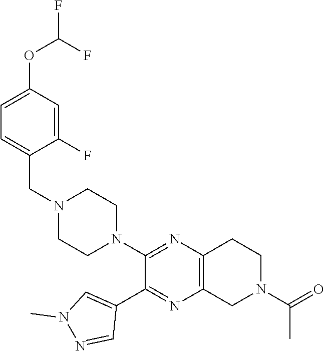 [SMILES: CC(=O)N1CCc2nc(N3CCN(Cc4ccc(OC(F)F)cc4F)CC3)c(-c3cnn(C)c3)nc2C1] | 1-[2-(4-{[4- (difluoromethoxy)-2- fluorophenyl]methyl} piperazin-1-yl)-3-(1-methyl- 1H-pyrazol-4-yl)- 5H,6H,7H,8H-pyrido[3,4- b]pyrazin-6-yl]ethan-1- one | NA |
| 359 | 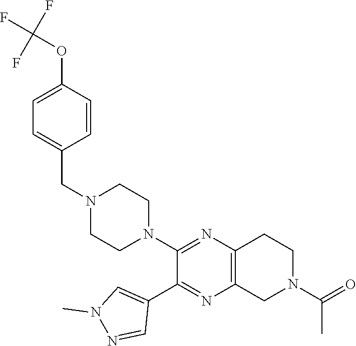 [SMILES: CC(=O)N1CCc2nc(N3CCN(Cc4ccc(OC(F)(F)F)cc4)CC3)c(-c3cnn(C)c3)nc2C1] | 1-[3-(1-methyl-1H- pyrazol-4-yl)-2-(4-{[4- (trifluoromethoxy)phenyl] methyl}piperazin-1-yl)- 5H,6H,7H,8H-pyrido [3,4- b]pyrazin-6-yl]ethan-1- one | NA |
| 360 | 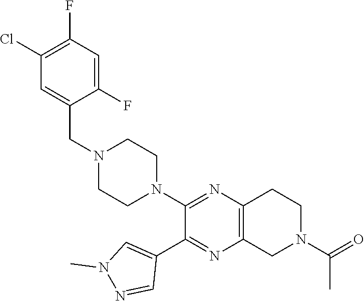 [SMILES: CC(=O)N1CCc2nc(N3CCN(Cc4cc(Cl)c(F)cc4F)CC3)c(-c3cnn(C)c3)nc2C1] | 1-(2-{4-[(5-chloro-2,4- difluorophenyl)methyl] piperazin-1-yl}-3-(1-methyl- 1H-pyrazol-4-yl)- 5H,6H,7H,8H-pyrido[3,4- b]pyrazin-6-yl)ethan-1- one | NA |
| 361 | 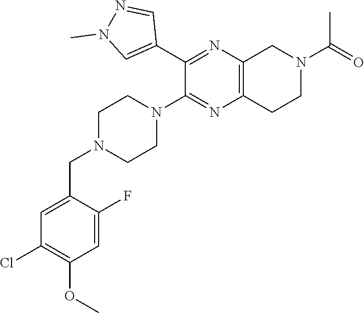 [SMILES: COc1cc(F)c(CN2CCN(c3nc4c(nc3-c3cnn(C)c3)CN(C(C)=O)CC4)CC2)cc1Cl] | 1-(2-{4-[(5-chloro-2- fluoro-4- methoxyphenyl)methyl] piperazin-1-yl}-3-(1-methyl- 1H-pyrazol-4-yl)- 5H,6H,7H,8H-pyrido[3,4- b]pyrazin-6-yl)ethan-1- one | NA |
| 362 | 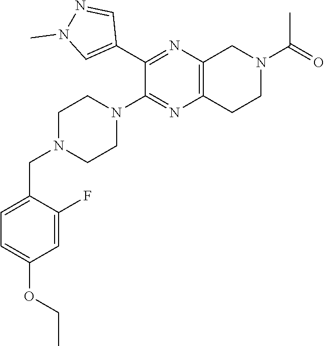 [SMILES: CCOc1ccc(CN2CCN(c3nc4c(nc3-c3cnn(C)c3)CN(C(C)=O)CC4)CC2)c(F)c1] | 1-(2-{4-[(4-ethoxy-2- fluorophenyl)methyl] piperazin-1-yl}-3-(1-methyl- 1H-pyrazol-4-yl)- 5H,6H,7H,8H-pyrido[3,4- b]pyrazin-6-yl)ethan-1- one | NA |
| 363 | 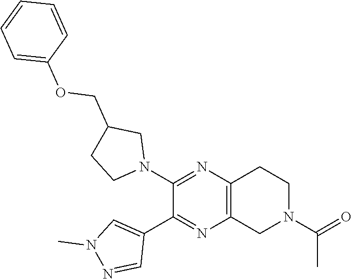 [SMILES: CC(=O)N1CCc2nc(N3CCC(COc4ccccc4)C3)c(-c3cnn(C)c3)nc2C1] | 1-[3-(1-methyl-1H- pyrazol-4-yl)-2-[3- (phenoxymethyl)pyrrolidin- 1-yl]-5H,6H,7H,8H- pyrido[3,4-b]pyrazin-6- yl]ethan-1-one | NA |
| 364 | 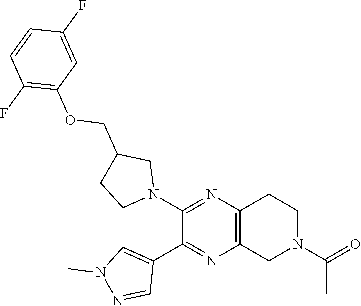 [SMILES: CC(=O)N1CCc2nc(N3CCC(COc4cc(F)ccc4F)C3)c(-c3cnn(C)c3)nc2C1] | 1-(2-{3-[(2,5- difluorophenoxy)methyl] pyrrolidin-1-yl}-3-(1- methyl-1H-pyrazol-4-yl)- 5H,6H,7H,8H-pyrido[3,4- b]pyrazin-6-yl)ethan-1- one | NA |
| 365 | 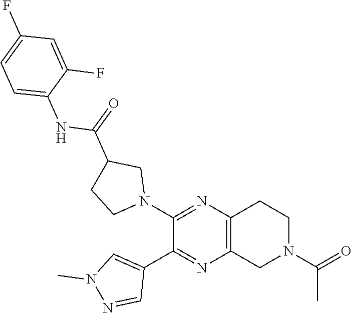 [SMILES: CC(=O)N1CCc2nc(N3CCC(C(=O)Nc4ccc(F)cc4F)C3)c(-c3cnn(C)c3)nc2C1] | 1-[6-acetyl-3-(1-methyl- 1H-pyrazol-4-yl)- 5H,6H,7H,8H-pyrido [3,4- b]pyrazin-2-yl]-N-(2,4- difluorophenyl)pyrrolidin e-3-carboxamide | NA |
| 366 | 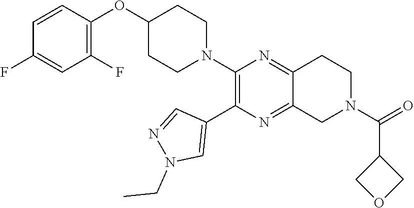 [SMILES: CCn1cc(-c2nc3c(nc2N2CCC(Oc4ccc(F)cc4F)CC2)CCN(C(=O)C2COC2)C3)cn1] | 4-(2,4-difluorophenoxy)- 1-[3-(1-ethyl-1H-pyrazol- 4-yl)-6-(oxetane-3- carbonyl)-5H,6H,7H,8H- pyrido[3,4-b]pyrazin-2- yl]piperidine | NA |
| 367 | 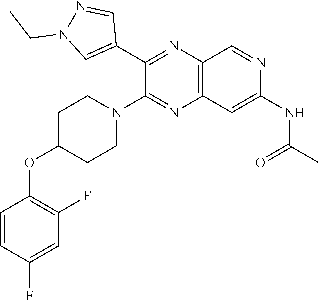 [SMILES: CCn1cc(-c2nc3cnc(NC(C)=O)cc3nc2N2CCC(Oc3ccc(F)cc3F)CC2)cn1] | N-{2-[4-(2,4- difluorophenoxy)piperidin- 1-yl]-3-(1-ethyl-1H- pyrazol-4-yl)pyrido[3,4- b]pyrazin-7-yl}acetamide | NA |
| 368 | 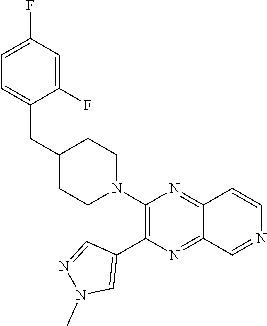 [SMILES: Cn1cc(-c2nc3cnccc3nc2N2CCC(Cc3ccc(F)cc3F)CC2)cn1] | 4-[(2,4- difluorophenyl)methyl]-1- [3-(1-methyl-1H-pyrazol- 4-yl)pyrido[3,4-b]pyrazin- 2-yl]piperidine | NA |
| 369 | 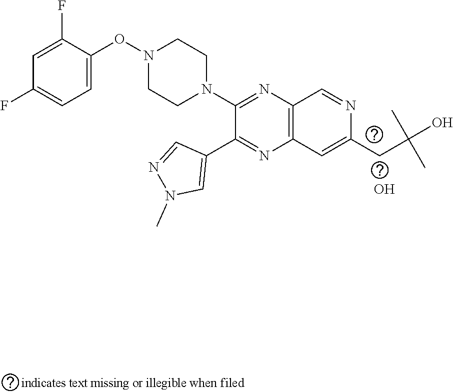 or enantiomer | rel-(1R)-1-(3-{4-[(2,4- difluorophenyl)methyl] piperazin-1-yl}-2-(1-methyl- 1H-pyrazol-4- yl)pyrido[3,4-b]pyrazin-7- yl)-2-methylpropane-1,2- diol, or enantiomer | (+) isomer |
| 370 | 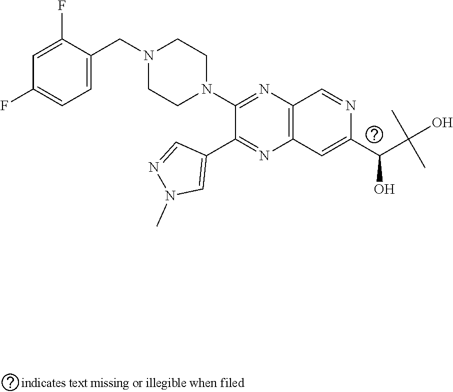 [SMILES: Cn1cc(-c2nc3cc([C@H](O)C(C)(C)O)ncc3nc2N2CCN(Cc3ccc(F)cc3F)CC2)cn1] or enantiomer | rel-(1R)-1-(3-{4-[(2,4- difluorophenyl)methyl] piperazin-1-yl}-2-(1-methyl- 1H-pyrazol-4- yl)pyrido[3,4-b]pyrazin-7- yl)-2-methylpropane-1,2- diol, or enantiomer | (−) isomer |
| 371 | 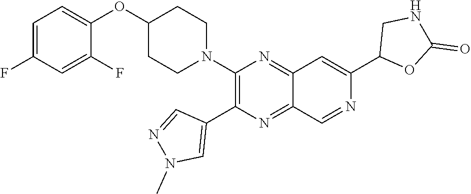 [SMILES: Cn1cc(-c2nc3cnc(C4CNC(=O)O4)cc3nc2N2CCC(Oc3ccc(F)cc3F)CC2)cn1] | 5-{2-[4-(2,4- difluorophenoxy)piperidin- 1-yl]-3-(1-methyl-1H- pyrazol-4-yl)pyrido[3,4- b]pyrazin-7-yl}-1,3- oxazolidin-2-one | NA |
| 372 | 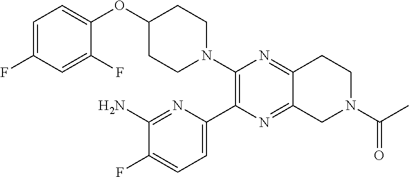 [SMILES: CC(=O)N1CCc2nc(N3CCC(Oc4ccc(F)cc4F)CC3)c(-c3ccc(F)c(N)n3)nc2C1] | 1-{3-(6-amino-5-fluoro-2- pyridyl)-2-[4-(2,4- difluorophenoxy)-1- piperidyl]-5,6,7,8- tetrahydro-1,4,6-triaza-6- naphthyl}-1-ethanone | NA |
| 373 | 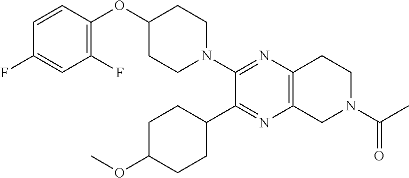 [SMILES: COC1CCC(c2nc3c(nc2N2CCC(Oc4ccc(F)cc4F)CC2)CCN(C(C)=O)C3)CC1] | 1-{2-[4-(2,4- difluorophenoxy)piperidin- 1-yl]-3-(4- methoxycyclohexyl)- 5H,6H,7H,8H-pyrido[3,4- b]pyrazin-6-yl}ethan-1- one | NA |
| 374 | 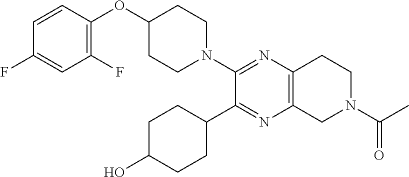 [SMILES: CC(=O)N1CCc2nc(N3CCC(Oc4ccc(F)cc4F)CC3)c(C3CCC(O)CC3)nc2C1] | 1-{2-[4-(2,4- difluorophenoxy)piperidin- 1-yl]-3-(4- hydroxycyclohexyl)- 5H,6H,7H,8H-pyrido[3,4- b]pyrazin-6-yl}ethan-1- one | NA |
| 375 | 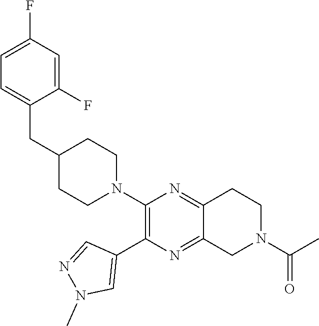 [SMILES: CC(=O)N1CCc2nc(N3CCC(Cc4ccc(F)cc4F)CC3)c(-c3cnn(C)c3)nc2C1] | 1-(2-{4-[(2,4- difluorophenyl)methyl] piperidin-1-yl}-3-(1-methyl- 1H-pyrazol-4-yl)- 5H,6H,7H,8H-pyrido[3,4- b]pyrazin-6-yl)ethan-1- one | NA |
| 376 | 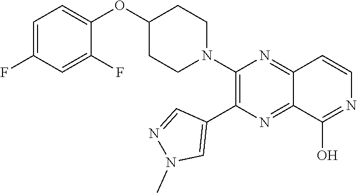 [SMILES: Cn1cc(-c2nc3c(O)nccc3nc2N2CCC(Oc3ccc(F)cc3F)CC2)cn1] | 2-[4-(2,4- difluorophenoxy)piperidin- 1-yl]-3-(1-methyl-1H- pyrazol-4-yl)pyrido[3,4- b]pyrazin-5-ol | NA |
| 377 | 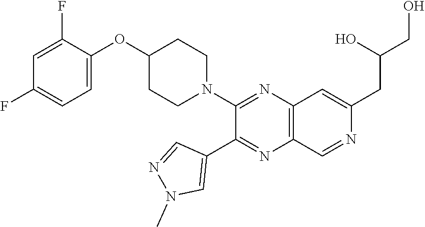 [SMILES: Cn1cc(-c2nc3cnc(CC(O)CO)cc3nc2N2CCC(Oc3ccc(F)cc3F)CC2)cn1] | 3-{2-[4-(2,4- difluorophenoxy)-1- piperidyl]-3-(1-methyl-4- pyrazolyl)-1,4,6-triaza-7- naphthyl}-1,2-propanediol | NA |
| 378 | 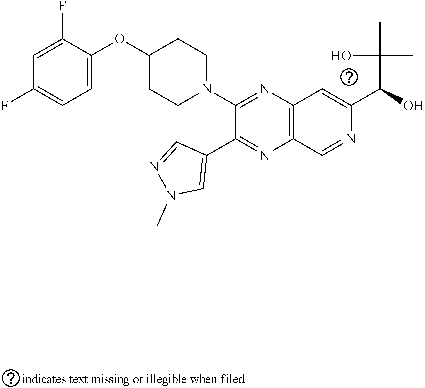 [SMILES: Cn1cc(-c2nc3cnc([C@H](O)C(C)(C)O)cc3nc2N2CCC(Oc3ccc(F)cc3F)CC2)cn1] or enantiomer | (S)-1-{2-[4-(2,4- difluorophenoxy)-1- piperidyl]-3-(1-methyl-4- pyrazolyl)-1,4,6-triaza-7- naphthyl}-2-methyl-1,2- propanediol, or enantiomer | (−) isomer |
| 379 | 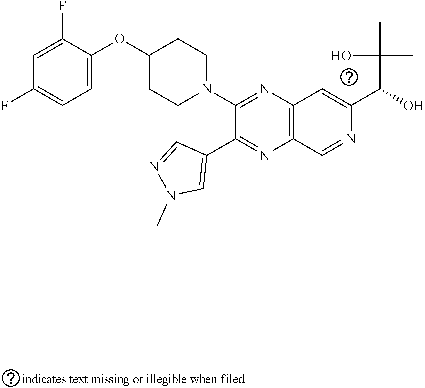 [SMILES: Cn1cc(-c2nc3cnc([C@@H](O)C(C)(C)O)cc3nc2N2CCC(Oc3ccc(F)cc3F)CC2)cn1] or enantiomer | (S)-1-{2-[4-(2,4- difluorophenoxy)-1- piperidyl]-3-(1-methyl-4- pyrazolyl)-1,4,6-triaza-7- naphthyl}-2-methyl-1,2- propanediol, or enantiomer | (+) isomer |
| 380 | 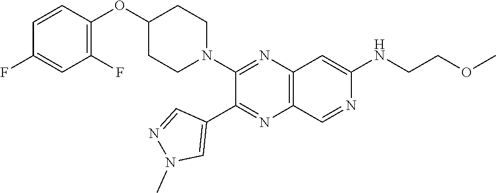 [SMILES: COCCNc1cc2nc(N3CCC(Oc4ccc(F)cc4F)CC3)c(-c3cnn(C)c3)nc2cn1] | 2-[4-(2,4- difluorophenoxy)piperidin- 1-yl]-N-(2- methoxyethyl)-3-(1- methyl-1H-pyrazol-4- yl)pyrido[3,4-b]pyrazin-7- amine | NA |
| 381 | 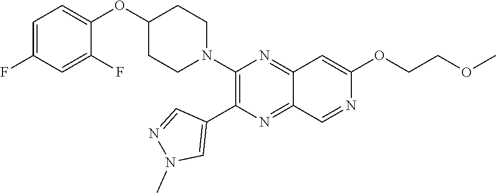 [SMILES: COCCOc1cc2nc(N3CCC(Oc4ccc(F)cc4F)CC3)c(-c3cnn(C)c3)nc2cn1] | 4-(2,4-difluorophenoxy)- 1-[7-(2-methoxyethoxy)- 3-(1-methyl-1H-pyrazol-4- yl)pyrido[3,4-b]pyrazin-2- yl]piperidine | NA |
| 382 | 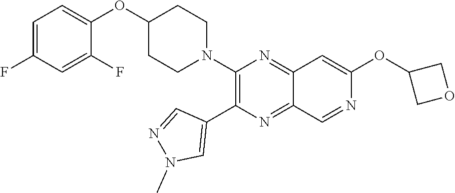 [SMILES: Cn1cc(-c2nc3cnc(OC4COC4)cc3nc2N2CCC(Oc3ccc(F)cc3F)CC2)cn1] | 4-(2,4-difluorophenoxy)- 1-[3-(1-methyl-1H- pyrazol-4-yl)-7-(oxetan-3- yloxy)pyrido[3,4- b]pyrazin-2-yl]piperidine | NA |
| 383 | 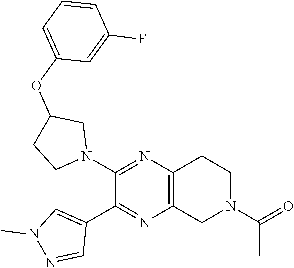 [SMILES: CC(=O)N1CCc2nc(N3CCC(Oc4cccc(F)c4)C3)c(-c3cnn(C)c3)nc2C1] | 1-{2-[3-(3- fluorophenoxy)pyrrolidin- 1-yl]-3-(1-methyl-1H- pyrazol-4-yl)- 5H,6H,7H,8H-pyrido[3,4- b]pyrazin-6-yl}ethan-1- one | NA |
| 384 | 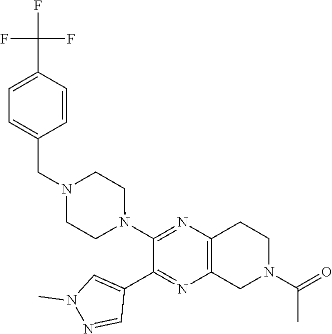 [SMILES: CC(=O)N1CCc2nc(N3CCN(Cc4ccc(C(F)(F)F)cc4)CC3)c(-c3cnn(C)c3)nc2C1] | 1-[3-(1-methyl-1H- pyrazol-4-yl)-2-(4-{[4- (trifluoromethyl)phenyl]m ethyl}piperazin-1-yl)- 5H,6H,7H,8H-pyrido[3,4- b]pyrazin-6-yl]ethan-1- one | NA |
| 385 | 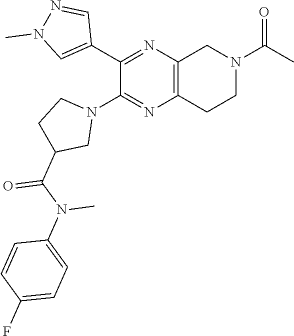 [SMILES: CC(=O)N1CCc2nc(N3CCC(C(=O)N(C)c4ccc(F)cc4)C3)c(-c3cnn(C)c3)nc2C1] | 1-[6-acetyl-3-(1-methyl- 1H-pyrazol-4-yl)- 5H,6H,7H,8H-pyrido[3,4- b]pyrazin-2-yl]-N-(4- fluorophenyl)-N- methylpyrrolidine-3- carboxamide | NA |
| 386 | 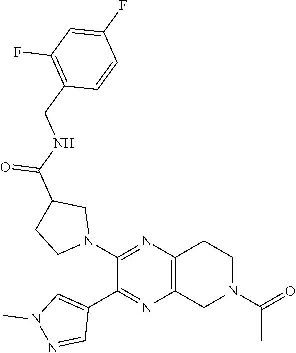 [SMILES: CC(=O)N1CCc2nc(N3CCC(C(=O)NCc4ccc(F)cc4F)C3)c(-c3cnn(C)c3)nc2C1] | 1-[6-acetyl-3-(1-methyl- 1H-pyrazol-4-yl)- 5H,6H,7H,8H-pyrido[3,4- b]pyrazin-2-yl]-N-[(2,4- difluorophenyl)methyl] pyrrolidine-3-carboxamide | NA |
| 387 | 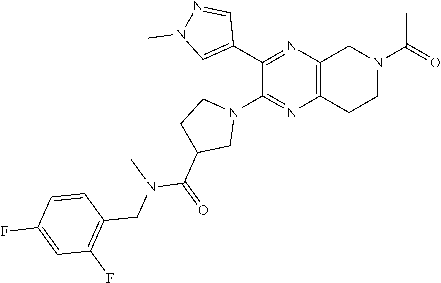 [SMILES: CC(=O)N1CCc2nc(N3CCC(C(=O)N(C)Cc4ccc(F)cc4F)C3)c(-c3cnn(C)c3)nc2C1] | 1-[6-acetyl-3-(1-methyl- 1H-pyrazol-4-yl)- 5H,6H,7H,8H-pyrido[3,4- b]pyrazin-2-yl]-N-[(2,4- difluorophenyl)methyl]-N- methylpyrrolidine-3- carboxamide | NA |
| 388 | 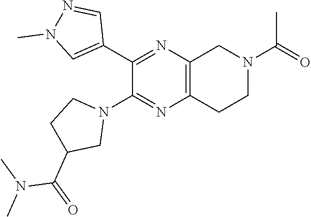 [SMILES: CC(=O)N1CCc2nc(N3CCC(C(=O)N(C)C)C3)c(-c3cnn(C)c3)nc2C1] | 1-[6-acetyl-3-(1-methyl- 1H-pyrazol-4-yl)- 5H,6H,7H,8H-pyrido[3,4- b]pyrazin-2-yl]-N,N- dimethylpyrrolidine-3- carboxamide | NA |
| 389 | 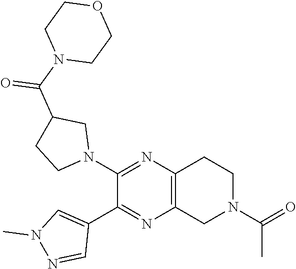 [SMILES: CC(=O)N1CCc2nc(N3CCC(C(=O)N4CCOCC4)C3)c(-c3cnn(C)c3)nc2C1] | 1-[3-(1-methyl-1H- pyrazol-4-yl)-2-[3- (morpholine-4- carbonyl)pyrrolidin-1-yl]- 5H,6H,7H,8H-pyrido[3,4- b]pyrazin-6-yl]ethan-1- one | NA |
| 390 | 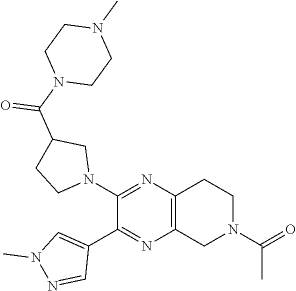 [SMILES: CC(=O)N1CCc2nc(N3CCC(C(=O)N4CCN(C)CC4)C3)c(-c3cnn(C)c3)nc2C1] | 4-{1-[6-acetyl-3-(1- methyl-1H-pyrazol-4-yl)- 5H,6H,7H,8H-pyrido[3,4- b]pyrazin-2- yl]pyrrolidine-3- carbonyl}-1- methylpiperazin-2-one | NA |
| 391 | 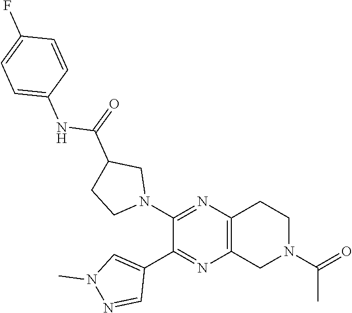 [SMILES: CC(=O)N1CCc2nc(N3CCC(C(=O)Nc4ccc(F)cc4)C3)c(-c3cnn(C)c3)nc2C1] | 1-[6-acetyl-3-(1-methyl- 1H-pyrazol-4-yl)- 5H,6H,7H,8H-pyrido[3,4- b]pyrazin-2-yl]-N-(4- fluorophenyl)pyrrolidine- 3-carboxamide | NA |
| 392 | 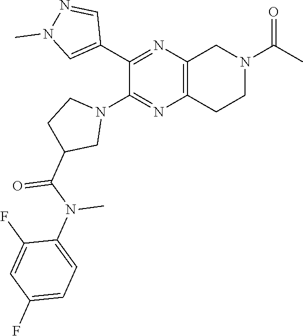 [SMILES: CC(=O)N1CCc2nc(N3CCC(C(=O)N(C)c4ccc(F)cc4F)C3)c(-c3cnn(C)c3)nc2C1] | 1-[6-acetyl-3-(1-methyl- 1H-pyrazol-4-yl)- 5H,6H,7H,8H-pyrido[3,4- b]pyrazin-2-yl]-N-(2,4- difluorophenyl)-N- methylpyrrolidine-3- carboxamide | NA |
| 393 | 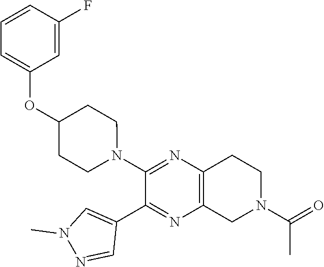 [SMILES: CC(=O)N1CCc2nc(N3CCC(Oc4cccc(F)c4)CC3)c(-c3cnn(C)c3)nc2C1] | 1-{2-[4-(3- fluorophenoxy)piperidin- 1-yl]-3-(1-methyl-1H- pyrazol-4-yl)- 5H,6H,7H,8H-pyrido[3,4- b]pyrazin-6-yl}ethan-1- one | NA |
| 394 | 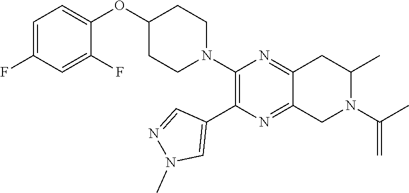 [SMILES: C=C(C)N1Cc2nc(-c3cnn(C)c3)c(N3CCC(Oc4ccc(F)cc4F)CC3)nc2CC1C] | 1-{2-[4-(2,4- difluorophenoxy)piperidin- 1-yl]-7-methyl-3-(1- methyl-1H-pyrazol-4-yl)- 5H,6H,7H,8H-pyrido[3,4- b]pyrazin-6-yl}ethan-1- one | NA |
| 395 | 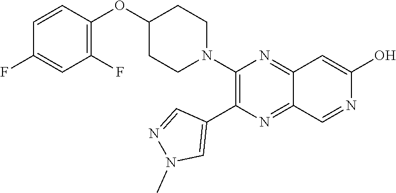 [SMILES: Cn1cc(-c2nc3cnc(O)cc3nc2N2CCC(Oc3ccc(F)cc3F)CC2)cn1] | 2-[4-(2,4- difluorophenoxy)piperidin- 1-yl]-3-(1-methyl-1H- pyrazol-4-yl)pyrido[3,4- b]pyrazin-7-ol | NA |
| 396 | 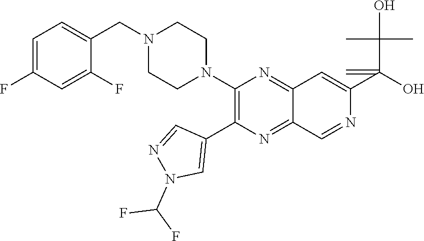 or enantiomer | (1R)-1-{3-[1- (difluoromethyl)-1H- pyrazol-4-yl]-2-{4-[(2,4- difluorophenyl)methyl] piperazin-1-yl}pyrido[3,4- b]pyrazin-7-yl}-2- methylpropane-1,2-diol, or enantiomer | (+) Isomer |
| 397 | 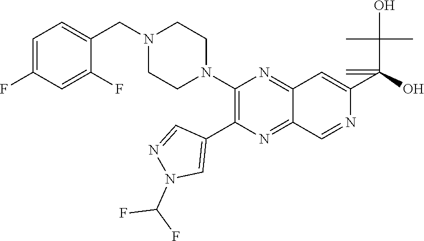 or enantiomer | (1S)-1-{3-[1- (difluoromethyl)-1H- pyrazol-4-yl]-2-{4-[(2,4- difluorophenyl)methyl] piperazin-1-yl}pyrido[3,4- b]pyrazin-7-yl}-2- methylpropane-1,2-diol, or enantiomer | (−) Isomer |
| 398 | 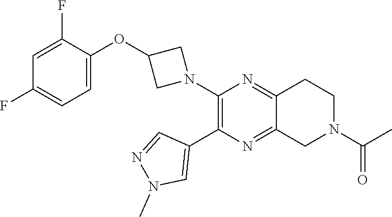 [SMILES: CC(=O)N1CCc2nc(N3CC(Oc4ccc(F)cc4F)C3)c(-c3cnn(C)c3)nc2C1] | 1-{2-[3-(2,4- difluorophenoxy)azetidin- 1-yl]-3-(1-methyl-1H- pyrazol-4-yl)- 5H,6H,7H,8H-pyrido[3,4- b]pyrazin-6-yl}ethan-1- one | NA |
| 399 | 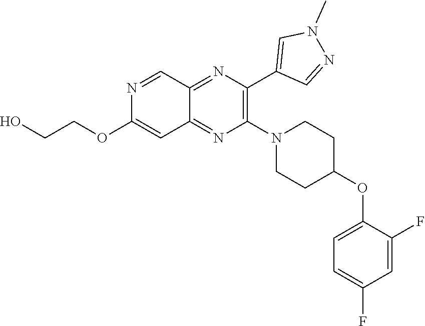 [SMILES: Cn1cc(-c2nc3cnc(OCCO)cc3nc2N2CCC(Oc3ccc(F)cc3F)CC2)cn1] | 2-({2-[4-(2,4- difluorophenoxy)piperidin- 1-yl]-3-(1-methyl-1H- pyrazol-4-yl)pyrido[3,4- b]pyrazin-7-yl}oxy)ethan- 1-ol | NA |
| 400 | 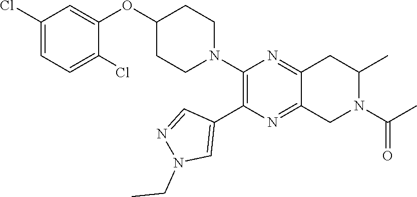 [SMILES: CCn1cc(-c2nc3c(nc2N2CCC(Oc4cc(Cl)ccc4Cl)CC2)CC(C)N(C(C)=O)C3)cn1] | 1-{2-[4-(2,5- dichlorophenoxy)piperidin- 1-yl]-3-(1-ethyl-1H- pyrazol-4-yl)- 5H,6H,7H,8H-pyrido[3,4- b]pyrazin-6-yl}ethan-1- one | NA |
| 401 | 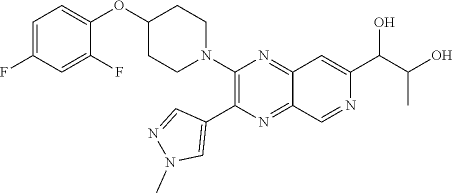 [SMILES: CC(O)C(O)c1cc2nc(N3CCC(Oc4ccc(F)cc4F)CC3)c(-c3cnn(C)c3)nc2cn1] | 1-{2-[4-(2,4- difluorophenoxy)piperidin- 1-yl]-3-(1-methyl-1H- pyrazol-4-yl)pyrido[3,4- b]pyrazin-7-yl}propane- 1,2-diol | NA |
| 402 | 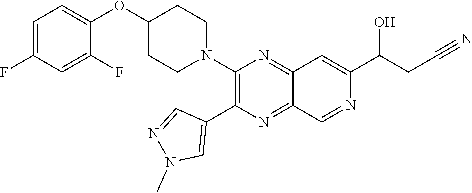 [SMILES: Cn1cc(-c2nc3cnc(C(O)CC#N)cc3nc2N2CCC(Oc3ccc(F)cc3F)CC2)cn1] | 3-{2-[4-(2,4- difluorophenoxy)piperidin- 1-yl]-3-(1-methyl-1H- pyrazol-4-yl)pyrido[3,4- b]pyrazin-7-yl}-3- hydroxypropanenitrile | NA |
| 403 | 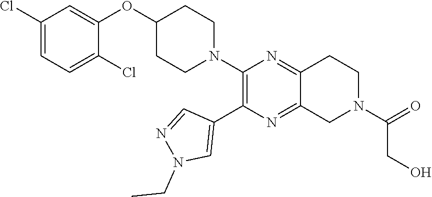 [SMILES: CCn1cc(-c2nc3c(nc2N2CCC(Oc4cc(Cl)ccc4Cl)CC2)CCN(C(=O)CO)C3)cn1] | 1-{2-[4-(2,5- dichlorophenoxy)piperidin- 1-yl]-3-(1-ethyl-1H- pyrazol-4-yl)- 5H,6H,7H,8H-pyrido[3,4- b]pyrazin-6-yl}-2- hydroxyethan-1-one | NA |
| 404 | 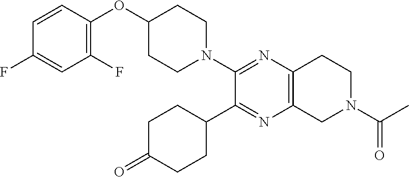 [SMILES: CC(=O)N1CCc2nc(N3CCC(Oc4ccc(F)cc4F)CC3)c(C3CCC(=O)CC3)nc2C1] | 4-{6-acetyl-2-[4-(2,4- difluorophenoxy)piperidin- 1-yl]-5H,6H,7H,8H- pyrido[3,4-b]pyrazin-3- yl}cyclohexan-1-one | NA |
| 405 | 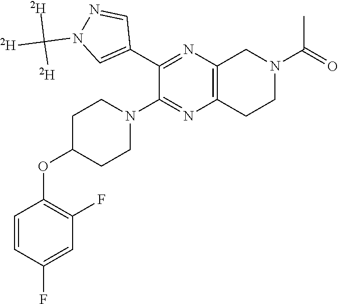 [SMILES: [2H]C([2H])([2H])n1cc(-c2nc3c(nc2N2CCC(Oc4ccc(F)cc4F)CC2)CCN(C(C)=O)C3)cn1] | 1-{2-[4-(2,4- difluorophenoxy)piperidin- 1-yl]-3-[1-(2H3)methyl- 1H-pyrazol-4-yl]- 5H,6H,7H,8H-pyrido[3,4- b]pyrazin-6-yl}ethan-1- one | NA |
| 406 | 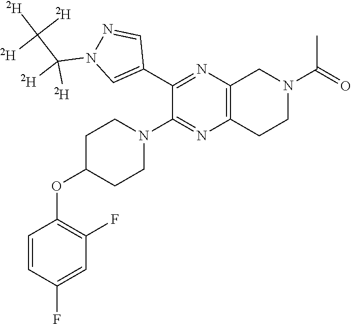 [SMILES: [2H]C([2H])([2H])C([2H])([2H])n1cc(-c2nc3c(nc2N2CCC(Oc4ccc(F)cc4F)CC2)CCN(C(C)=O)C3)cn1] | 1-{2-[4-(2,4- difluorophenoxy)piperidin- 1-yl]-3-{1-[(1,1,2,2,2- 2H5)ethyl]-1H-pyrazol-4- yl}-5H,6H,7H,8H- pyrido[3,4-b]pyrazin-6- yl}ethan-1-one | NA |
| 407 | 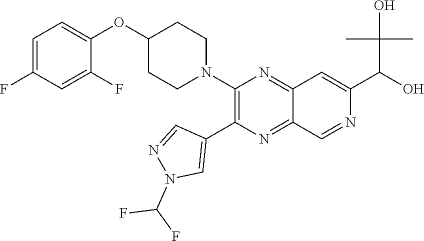 [SMILES: CC(C)(O)C(O)c1cc2nc(N3CCC(Oc4ccc(F)cc4F)CC3)c(-c3cnn(C(F)F)c3)nc2cn1] | 1-{3-[1-(difluoromethyl)- 4-pyrazolyl]-2-[4-(2,4- difluorophenoxy)-1- piperidyl]-1,4,6-triaza-7- naphthyl}-2-methyl-1,2- propanediol | NA |
| 408 | 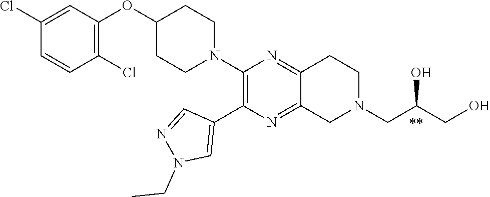 [SMILES: CCn1cc(-c2nc3c(nc2N2CCC(Oc4cc(Cl)ccc4Cl)CC2)CCN(C[C@@H](O)CO)C3)cn1] or enantiomer | (2R)-3-{2-[4-(2,5- dichlorophenoxy)piperidin- 1-yl]-3-(1-ethyl-1H- pyrazol-4-yl)- 5H,6H,7H,8H-pyrido[3,4- b]pyrazin-6-yl}propane- 1,2-diol, or enantiomer | NA |
| 409 | 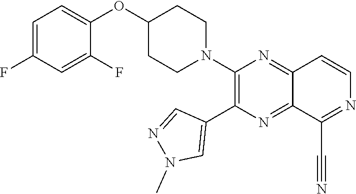 [SMILES: Cn1cc(-c2nc3c(C#N)nccc3nc2N2CCC(Oc3ccc(F)cc3F)CC2)cn1] | 2-[4-(2,4- difluorophenoxy)piperidin- 1-yl]-3-(1-methyl-1H- pyrazol-4-yl)pyrido[3,4- b]pyrazine-5-carbonitrile | NA |
| 410 | 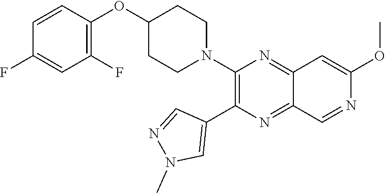 [SMILES: COc1cc2nc(N3CCC(Oc4ccc(F)cc4F)CC3)c(-c3cnn(C)c3)nc2cn1] | 4-(2,4-difluorophenoxy)- 1-[7-methoxy-3-(1- methyl-1H-pyrazol-4- yl)pyrido[3,4-b]pyrazin-2- yl]piperidine | NA |
| 411 | 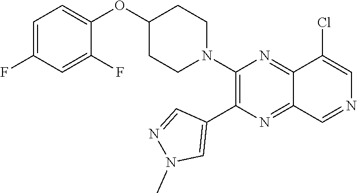 [SMILES: Cn1cc(-c2nc3cncc(Cl)c3nc2N2CCC(Oc3ccc(F)cc3F)CC2)cn1] | 1-[8-chloro-3-(1-methyl- 1H-pyrazol-4- yl)pyrido[3,4-b]pyrazin-2- yl]-4-(2,4- difluorophenoxy)piperidine | NA |
| 412 | 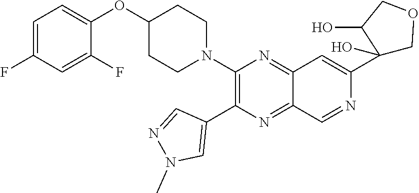 [SMILES: Cn1cc(-c2nc3cnc(C4(O)COCC4O)cc3nc2N2CCC(Oc3ccc(F)cc3F)CC2)cn1] | 3-{2-[4-(2,4- difluorophenoxy)piperidin- 1-yl]-3-(1-methyl-1H- pyrazol-4-yl)pyrido[3,4- b]pyrazin-7-yl}oxolane- 3,4-diol | NA |
| 413 | 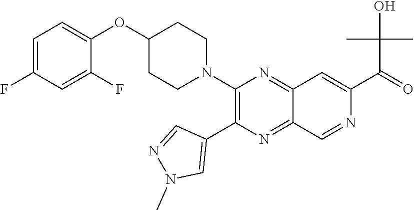 [SMILES: Cn1cc(-c2nc3cnc(C(=O)C(C)(C)O)cc3nc2N2CCC(Oc3ccc(F)cc3F)CC2)cn1] | 1-{2-[4-(2,4- difluorophenoxy)piperidin- 1-yl]-3-(1-methyl-1H- pyrazol-4-yl)pyrido[3,4- b]pyrazin-7-yl}-2- hydroxy-2-methylpropan- 1-one | NA |
| 414 | 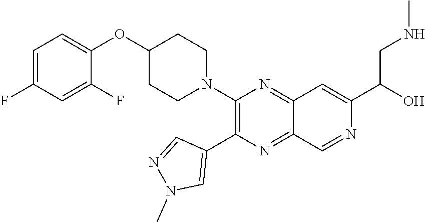 [SMILES: CNCC(O)c1cc2nc(N3CCC(Oc4ccc(F)cc4F)CC3)c(-c3cnn(C)c3)nc2cn1] | 1-{2-[4-(2,4- difluorophenoxy)piperidin- 1-yl]-3-(1-methyl-1H- pyrazol-4-yl)pyrido[3,4- b]pyrazin-7-yl}-2- (methylamino)ethan-1-ol | NA |
| 415 | 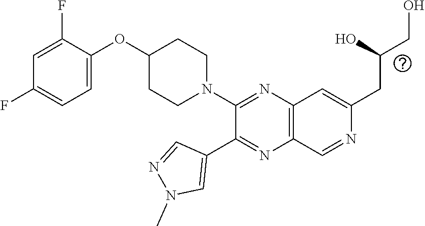 [SMILES: Cn1cc(-c2nc3cnc(C[C@@H](O)CO)cc3nc2N2CCC(Oc3ccc(F)cc3F)CC2)cn1] or enantiomer | (2R)-3-{2-[4-(2,4- difluorophenoxy)piperidin- 1-yl]-3-(1-methyl-1H- pyrazol-4-yl)pyrido[3,4- b]pyrazin-7-yl}propane- 1,2-diol, or enantiomer | (+) isomer |
| 416 | 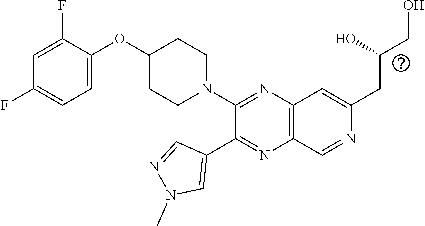 [SMILES: Cn1cc(-c2nc3cnc(C[C@H](O)CO)cc3nc2N2CCC(Oc3ccc(F)cc3F)CC2)cn1] or enantiomer | (2S)-3-{2-[4-(2,4- difluorophenoxy)piperidin- 1-yl]-3-(1-methyl-1H- pyrazol-4-yl)pyrido[3,4- b]pyrazin-7-yl}propane- 1,2-diol, or enantiomer | (−) isomer |
| 417 | 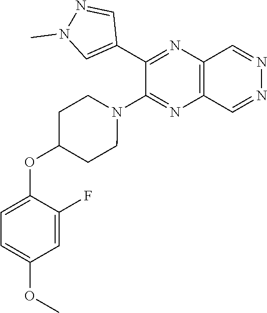 [SMILES: COc1ccc(OC2CCN(c3nc4cnncc4nc3-c3cnn(C)c3)CC2)c(F)c1] | 4-(2-fluoro-4- methoxyphenoxy)-1-[3-(1- methyl-1H-pyrazol-4- yl)pyrazino[2,3- d]pyridazin-2- yl]piperidine | NA |
| 418 | 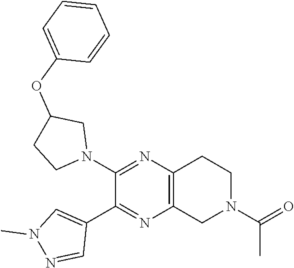 [SMILES: CC(=O)N1CCc2nc(N3CCC(Oc4ccccc4)C3)c(-c3cnn(C)c3)nc2C1] | 1-[3-(1-methyl-1H- pyrazol-4-yl)-2-(3- phenoxypyrrolidin-1-yl)- 5H,6H,7H,8H-pyrido[3,4- b]pyrazin-6-yl]ethan-1- one | NA |
| 419 | 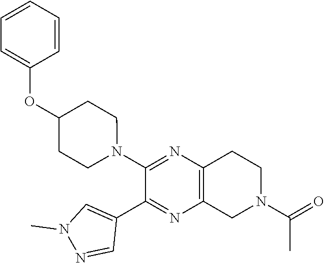 [SMILES: CC(=O)N1CCc2nc(N3CCC(Oc4ccccc4)CC3)c(-c3cnn(C)c3)nc2C1] | 1-[3-(1-methyl-1H- pyrazol-4-yl)-2-(4- phenoxypiperidin-1-yl)- 5H,6H,7H,8H-pyrido[3,4- b]pyrazin-6-yl]ethan-1- one | NA |
| 420 | 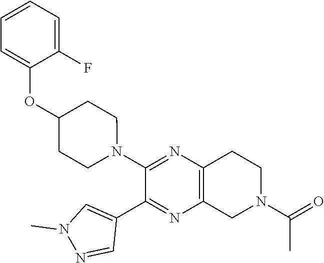 [SMILES: CC(=O)N1CCc2nc(N3CCC(Oc4ccccc4F)CC3)c(-c3cnn(C)c3)nc2C1] | 1-{2-[4-(2- fluorophenoxy)piperidin- 1-yl]-3-(1-methyl-1H- pyrazol-4-yl)- 5H,6H,7H,8H-pyrido[3,4- b]pyrazin-6-yl}ethan-1- one | NA |
| 421 | 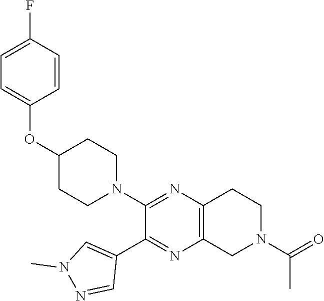 [SMILES: CC(=O)N1CCc2nc(N3CCC(Oc4ccc(F)cc4)CC3)c(-c3cnn(C)c3)nc2C1] | 1-{2-[4-(4- fluorophenoxy)piperidin- 1-yl]-3-(1-methyl-1H- pyrazol-4-yl)- 5H,6H,7H,8H-pyrido[3,4- b]pyrazin-6-yl}ethan-1- one | NA |
| 422 | 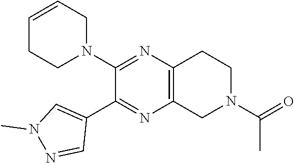 [SMILES: CC(=O)N1CCc2nc(N3CC=CCC3)c(-c3cnn(C)c3)nc2C1] | 1-[3-(1-methyl-1H- pyrazol-4-yl)-2-(1,2,3,6- tetrahydropyridin-1-yl)- 5H,6H,7H,8H-pyrido[3,4- b]pyrazin-6-yl]ethan-1- one | NA |
| 423 | 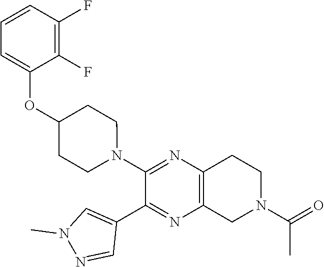 [SMILES: CC(=O)N1CCc2nc(N3CCC(Oc4cccc(F)c4F)CC3)c(-c3cnn(C)c3)nc2C1] | 1-{2-[4-(2,3- difluorophenoxy)piperidin- 1-yl]-3-(1-methyl-1H- pyrazol-4-yl)- 5H,6H,7H,8H-pyrido[3,4- b]pyrazin-6-yl}ethan-1- one | NA |
| 424 | 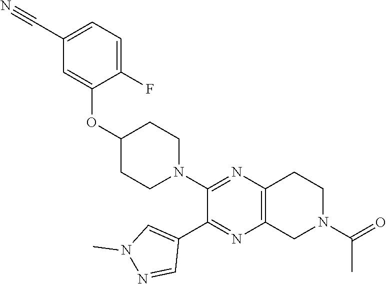 [SMILES: CC(=O)N1CCc2nc(N3CCC(Oc4cc(C#N)ccc4F)CC3)c(-c3cnn(C)c3)nc2C1] | 3-({1-[6-acetyl-3-(1- methyl-1H-pyrazol-4-yl)- 5H,6H,7H,8H-pyrido[3,4- b]pyrazin-2-yl]piperidin- 4-yl}oxy)-4- fluorobenzonitrile | NA |
| 425 | 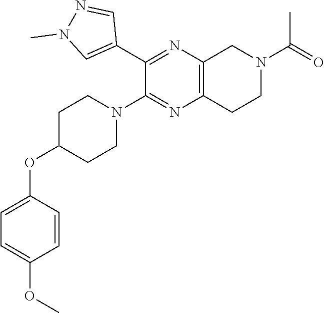 [SMILES: COc1ccc(OC2CCN(c3nc4c(nc3-c3cnn(C)c3)CN(C(C)=O)CC4)CC2)cc1] | 1-{2-[4-(4- methoxyphenoxy)piperidin- 1-yl]-3-(1-methyl-1H- pyrazol-4-yl)- 5H,6H,7H,8H-pyrido[3,4- b]pyrazin-6-yl}ethan-1- one | NA |
| 426 | 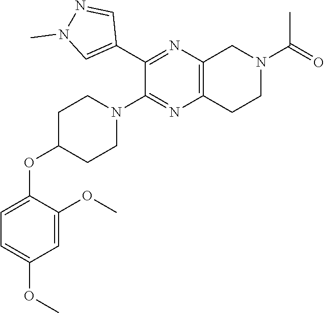 [SMILES: COc1ccc(OC2CCN(c3nc4c(nc3-c3cnn(C)c3)CN(C(C)=O)CC4)CC2)c(OC)c1] | 1-{2-[4-(2,4- dimethoxyphenoxy)piperi din-1-yl]-3-(1-methyl-1H- pyrazol-4-yl)- 5H,6H,7H,8H-pyrido[3,4- b]pyrazin-6-yl}ethan-1- one | NA |
| 427 | 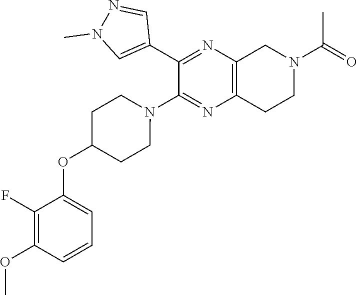 [SMILES: COc1cccc(OC2CCN(c3nc4c(nc3-c3cnn(C)c3)CN(C(C)=O)CC4)CC2)c1F] | 1-{2-[4-(2-fluoro-3- methoxyphenoxy)piperidin- 1-yl]-3-(1-methyl-1H- pyrazol-4-yl)- 5H,6H,7H,8H-pyrido[3,4- b]pyrazin-6-yl}ethan-1- one | NA |
| 428 | 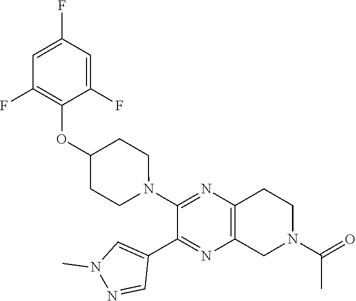 [SMILES: CC(=O)N1CCc2nc(N3CCC(Oc4c(F)cc(F)cc4F)CC3)c(-c3cnn(C)c3)nc2C1] | 1-[3-(1-methyl-1H- pyrazol-4-yl)-2-[4-(2,4,6- trifluorophenoxy)piperidin- 1-yl]-5H,6H,7H,8H- pyrido[3,4-b]pyrazin-6- yl]ethan-1-one | NA |
| 429 | 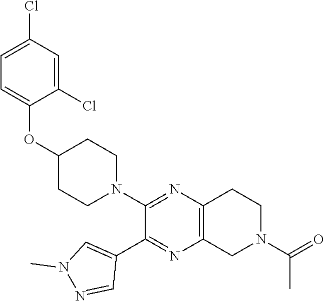 [SMILES: CC(=O)N1CCc2nc(N3CCC(Oc4ccc(Cl)cc4Cl)CC3)c(-c3cnn(C)c3)nc2C1] | 1-{2-[4-(2,4- dichlorophenoxy)piperidin- 1-yl]-3-(1-methyl-1H- pyrazol-4-yl)- 5H,6H,7H,8H-pyrido[3,4- b]pyrazin-6-yl}ethan-1- one | NA |
| 430 | 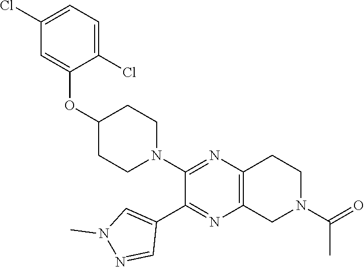 [SMILES: CC(=O)N1CCc2nc(N3CCC(Oc4cc(Cl)ccc4Cl)CC3)c(-c3cnn(C)c3)nc2C1] | 1-{2-[4-(2,5- dichlorophenoxy)piperidin- 1-yl]-3-(1-methyl-1H- pyrazol-4-yl)- 5H,6H,7H,8H-pyrido[3,4- b]pyrazin-6-yl}ethan-1- one | NA |
| 431 | 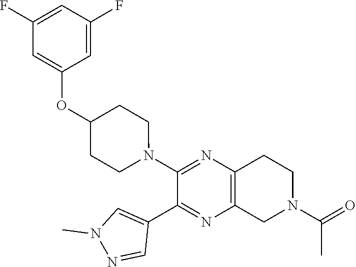 [SMILES: CC(=O)N1CCc2nc(N3CCC(Oc4cc(F)cc(F)c4)CC3)c(-c3cnn(C)c3)nc2C1] | 1-{2-[4-(3,5- difluorophenoxy)piperidin- 1-yl]-3-(1-methyl-1H- pyrazol-4-yl)- 5H,6H,7H,8H-pyrido[3,4- b]pyrazin-6-yl}ethan-1- one | NA |
| 432 | 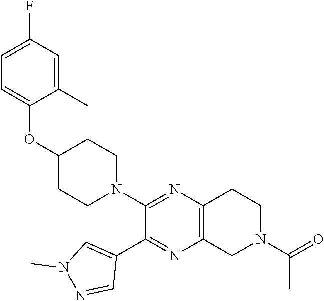 [SMILES: CC(=O)N1CCc2nc(N3CCC(Oc4ccc(F)cc4C)CC3)c(-c3cnn(C)c3)nc2C1] | 1-{2-[4-(4-fluoro-2- methylphenoxy)piperidin- 1-yl]-3-(1-methyl-1H- pyrazol-4-yl)- 5H,6H,7H,8H-pyrido[3,4- b]pyrazin-6-yl}ethan-1- one | NA |
| 433 | 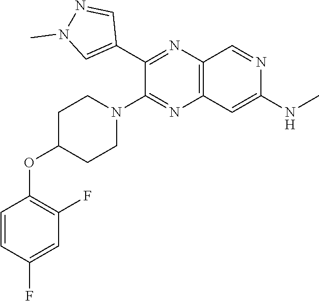 [SMILES: CNc1cc2nc(N3CCC(Oc4ccc(F)cc4F)CC3)c(-c3cnn(C)c3)nc2cn1] | 2-[4-(2,4- difluorophenoxy)piperidin- 1-yl]-N-methyl-3-(1- methyl-1H-pyrazol-4- yl)pyrido[3,4-b]pyrazin-7- amine | NA |
| 434 | 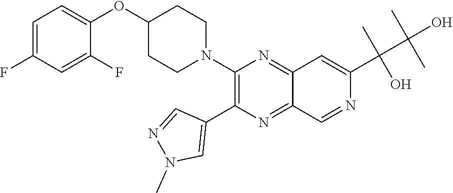 [SMILES: Cn1cc(-c2nc3cnc(C(C)(O)C(C)(C)O)cc3nc2N2CCC(Oc3ccc(F)cc3F)CC2)cn1] | 2-{2-[4-(2,4- difluorophenoxy)piperidin- 1-yl]-3-(1-methyl-1H- pyrazol-4-yl)pyrido[3,4- b]pyrazin-7-yl}-3- methylbutane-2,3-diol | NA |
| 435 | 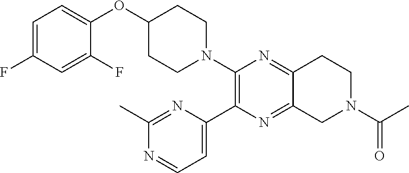 [SMILES: CC(=O)N1CCc2nc(N3CCC(Oc4ccc(F)cc4F)CC3)c(-c3ccnc(C)n3)nc2C1] | 1-{2-[4-(2,4- difluorophenoxy)piperidin- 1-yl]-3-(2- methylpyrimidin-4-yl)- 5H,6H,7H,8H-pyrido[3,4- b]pyrazin-6-yl}ethan-1- one | NA |
| 436 | 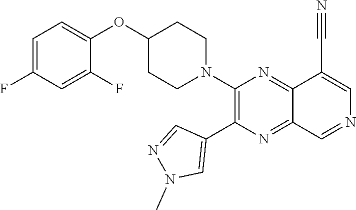 [SMILES: Cn1cc(-c2nc3cncc(C#N)c3nc2N2CCC(Oc3ccc(F)cc3F)CC2)cn1] | 2-[4-(2,4- difluorophenoxy)piperidin- 1-yl]-3-(1-methyl-1H- pyrazol-4-yl)pyrido[3,4- b]pyrazine-8-carbonitrile | NA |
| 437 | 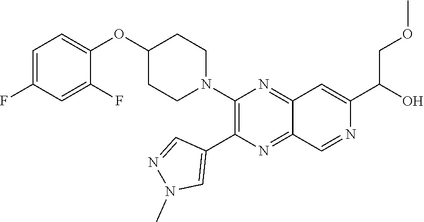 [SMILES: COCC(O)c1cc2nc(N3CCC(Oc4ccc(F)cc4F)CC3)c(-c3cnn(C)c3)nc2cn1] | 1-{2-[4-(2,4- difluorophenoxy)piperidin- 1-yl]-3-(1-methyl-1H- pyrazol-4-yl)pyrido[3,4- b]pyrazin-7-yl}-2- methoxyethan-1-ol | NA |
| 438 | 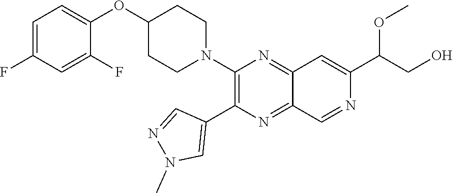 [SMILES: COC(CO)c1cc2nc(N3CCC(Oc4ccc(F)cc4F)CC3)c(-c3cnn(C)c3)nc2cn1] | 2-{2-[4-(2,4- difluorophenoxy)piperidin- 1-yl]-3-(1-methyl-1H- pyrazol-4-yl)pyrido[3,4- b]pyrazin-7-yl}-2- methoxyethan-1-ol | NA |
| 439 | 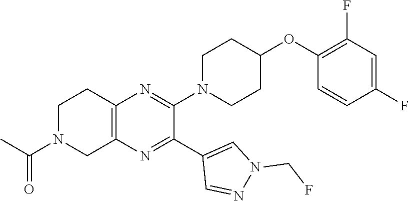 [SMILES: CC(=O)N1CCc2nc(N3CCC(Oc4ccc(F)cc4F)CC3)c(-c3cnn(CF)c3)nc2C1] | 1-{2-[4-(2,4- difluorophenoxy)piperidin- 1-yl]-3-[1-(fluoromethyl)- 1H-pyrazol-4-yl]- 5H,6H,7H,8H-pyrido[3,4- b]pyrazin-6-yl}ethan-1- one | NA |
| 440 | 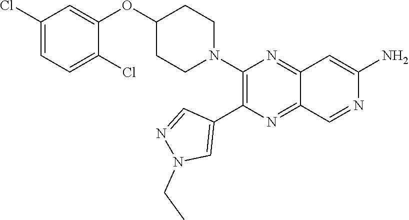 [SMILES: CCn1cc(-c2nc3cnc(N)cc3nc2N2CCC(Oc3cc(Cl)ccc3Cl)CC2)cn1] | 2-[4-(2,5- dichlorophenoxy)piperidin- 1-yl]-3-(1-ethyl-1H- pyrazol-4-yl)pyrido[3,4- b]pyrazin-7-amine | NA |
| 441 | 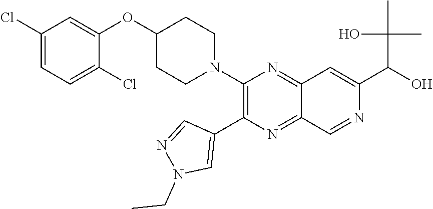 [SMILES: CCn1cc(-c2nc3cnc(C(O)C(C)(C)O)cc3nc2N2CCC(Oc3cc(Cl)ccc3Cl)CC2)cn1] | 1-{2-[4-(2,5- dichlorophenoxy)piperidin- 1-yl]-3-(1-ethyl-1H- pyrazol-4-yl)pyrido[3,4- b]pyrazin-7-yl}-2- methylpropane-1,2-diol | NA |
| 442 | 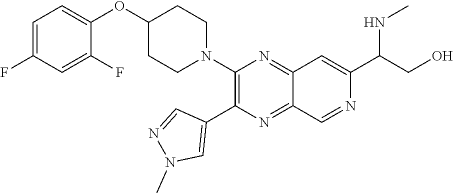 [SMILES: CNC(CO)c1cc2nc(N3CCC(Oc4ccc(F)cc4F)CC3)c(-c3cnn(C)c3)nc2cn1] | 2-{2-[4-(2,4- difluorophenoxy)piperidin- 1-yl]-3-(1-methyl-1H- pyrazol-4-yl)pyrido[3,4- b]pyrazin-7-yl}-2- (methylamino)ethan-1-ol | NA |
| 443 | 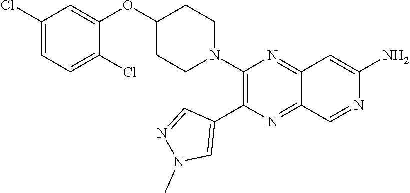 [SMILES: Cn1cc(-c2nc3cnc(N)cc3nc2N2CCC(Oc3cc(Cl)ccc3Cl)CC2)cn1] | 2-[4-(2,5- dichlorophenoxy)piperidin- 1-yl]-3-(1-methyl-1H- pyrazol-4-yl)pyrido[3,4- b]pyrazin-7-amine | NA |
| 444 | 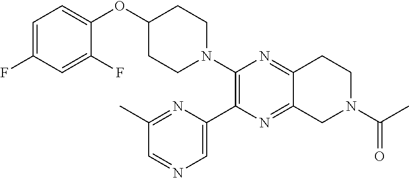 [SMILES: CC(=O)N1CCc2nc(N3CCC(Oc4ccc(F)cc4F)CC3)c(-c3cncc(C)n3)nc2C1] | 1-{2-[4-(2,4- difluorophenoxy)piperidin- 1-yl]-3-(6-methylpyrazin- 2-yl)-5H,6H,7H,8H- pyrido[3,4-b]pyrazin-6- yl}ethan-1-one | NA |
| 445 | 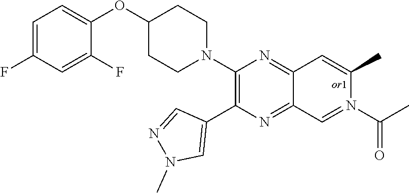 or enantiomer | rel-1-[(7R)-2-[4-(2,4- difluorophenoxy)piperidin- 1-yl]-7-methyl-3-(1- methyl-1H-pyrazol-4-yl)- 5H,6H,7H,8H-pyrido[3,4- b]pyrazin-6-yl]ethan-1- one, or enantiomer | NA |
| 446 | Or Enantiomer 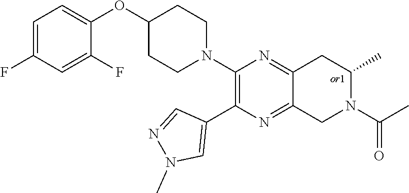 [SMILES: CC(=O)N1Cc2nc(-c3cnn(C)c3)c(N3CCC(Oc4ccc(F)cc4F)CC3)nc2C[C@@H]1C] or enantiomer | rel-1-[(7R)-2-[4-(2,4- difluorophenoxy)piperidin- 1-yl]-7-methyl-3-(1- methyl-1H-pyrazol-4-yl)- 5H,6H,7H,8H-pyrido[3,4- b]pyrazin-6-yl]ethan-1- one, or enantiomer | NA |
| 447 | 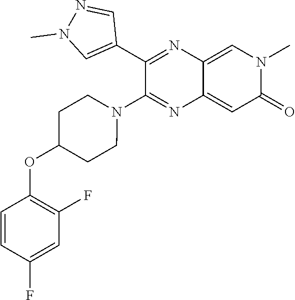 [SMILES: Cn1cc(-c2nc3cn(C)c(=O)cc3nc2N2CCC(Oc3ccc(F)cc3F)CC2)cn1] | 2-[4-(2,4- difluorophenoxy)piperidin- 1-yl]-6-methyl-3-(1- methyl-1H-pyrazol-4-yl)- 6H,7H-pyrido[3,4- b]pyrazin-7-one | NA |
| 448 | 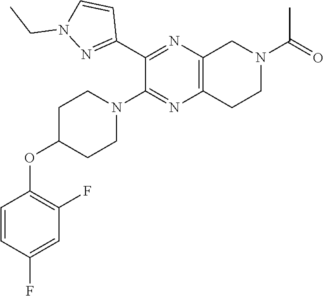 [SMILES: CCn1ccc(-c2nc3c(nc2N2CCC(Oc4ccc(F)cc4F)CC2)CCN(C(C)=O)C3)n1] | 1-{2-[4-(2,4- difluorophenoxy)piperidin- 1-yl]-3-(1-ethyl-1H- pyrazol-3-yl)- 5H,6H,7H,8H-pyrido[3,4- b]pyrazin-6-yl}ethan-1- one | NA |
| 449 | 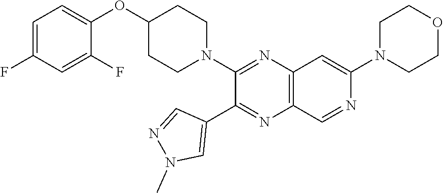 [SMILES: Cn1cc(-c2nc3cnc(N4CCOCC4)cc3nc2N2CCC(Oc3ccc(F)cc3F)CC2)cn1] | 4-{2-[4-(2,4- difluorophenoxy)piperidin- 1-yl]-3-(1-methyl-1H- pyrazol-4-yl)pyrido[3,4- b]pyrazin-7- yl}morpholine | NA |
| 450 | 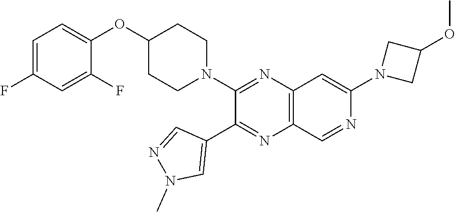 [SMILES: COC1CN(c2cc3nc(N4CCC(Oc5ccc(F)cc5F)CC4)c(-c4cnn(C)c4)nc3cn2)C1] | 4-(2,4-difluorophenoxy)- 1-[7-(3-methoxyazetidin- 1-yl)-3-(1-methyl-1H- pyrazol-4-yl)pyrido[3,4- b]pyrazin-2-yl]piperidine | NA |
| 451 | 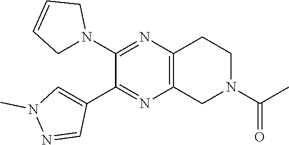 [SMILES: CC(=O)N1CCc2nc(N3CC=CC3)c(-c3cnn(C)c3)nc2C1] | 1-[2-(2,5-dihydro-1H- pyrrol-1-yl)-3-(1-methyl- 1H-pyrazol-4-yl)- 5H,6H,7H,8H-pyrido[3,4- b]pyrazin-6-yl]ethan-1- one | NA |
| 452 | 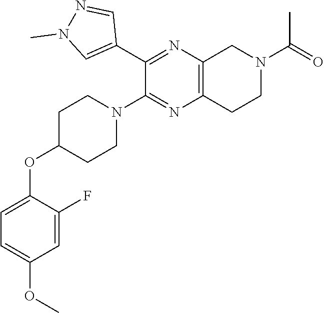 [SMILES: COc1ccc(OC2CCN(c3nc4c(nc3-c3cnn(C)c3)CN(C(C)=O)CC4)CC2)c(F)c1] | 1-{2-[4-(2-fluoro-4- methoxyphenoxy)piperidin- 1-yl]-3-(1-methyl-1H- pyrazol-4-yl)- 5H,6H,7H,8H-pyrido[3,4- b]pyrazin-6-yl}ethan-1- one | NA |
| 453 | 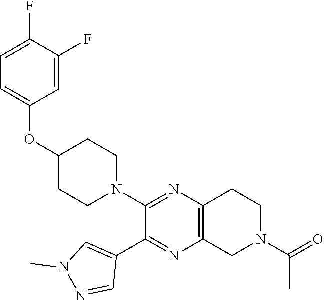 [SMILES: CC(=O)N1CCc2nc(N3CCC(Oc4ccc(F)c(F)c4)CC3)c(-c3cnn(C)c3)nc2C1] | 1-{2-[4-(3,4- difluorophenoxy)piperidin- 1-yl]-3-(1-methyl-1H- pyrazol-4-yl)- 5H,6H,7H,8H-pyrido[3,4- b]pyrazin-6-yl}ethan-1- one | NA |
| 454 | 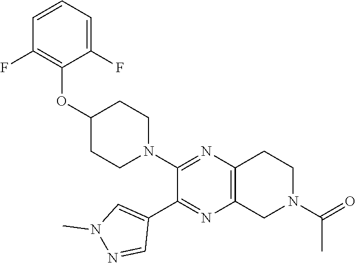 [SMILES: CC(=O)N1CCc2nc(N3CCC(Oc4c(F)cccc4F)CC3)c(-c3cnn(C)c3)nc2C1] | 1-{2-[4-(2,6- difluorophenoxy)piperidin- 1-yl]-3-(1-methyl-1H- pyrazol-4-yl)- 5H,6H,7H,8H-pyrido[3,4- b]pyrazin-6-yl}ethan-1- one | NA |
| 455 | 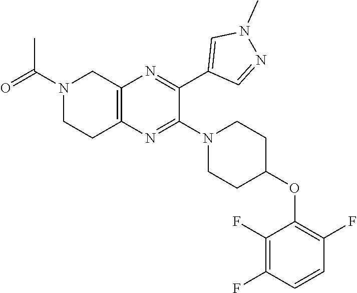 [SMILES: CC(=O)N1CCc2nc(N3CCC(Oc4c(F)ccc(F)c4F)CC3)c(-c3cnn(C)c3)nc2C1] | 1-[3-(1-methyl-1H- pyrazol-4-yl)-2-[4-(2,3,6- trifluorophenoxy)piperidin- 1-yl]-5H,6H,7H,8H- pyrido[3,4-b]pyrazin-6- yl]ethan-1-one | NA |
| 456 | 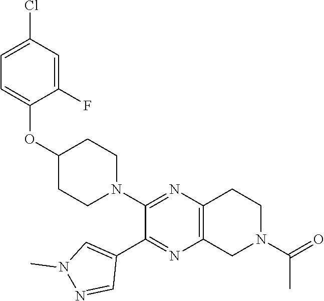 [SMILES: CC(=O)N1CCc2nc(N3CCC(Oc4ccc(Cl)cc4F)CC3)c(-c3cnn(C)c3)nc2C1] | 1-{2-[4-(4-chloro-2- fluorophenoxy)piperidin- 1-yl]-3-(1-methyl-1H- pyrazol-4-yl)- 5H,6H,7H,8H-pyrido[3,4- b]pyrazin-6-yl}ethan-1- one | NA |
| 457 | 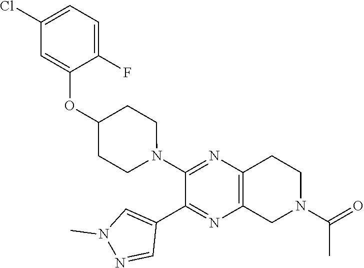 [SMILES: CC(=O)N1CCc2nc(N3CCC(Oc4cc(Cl)ccc4F)CC3)c(-c3cnn(C)c3)nc2C1] | 1-{2-[4-(5-chloro-2- fluorophenoxy)piperidin- 1-yl]-3-(1-methyl-1H- pyrazol-4-yl)- 5H,6H,7H,8H-pyrido[3,4- b]pyrazin-6-yl}ethan-1- one | NA |
| 458 | 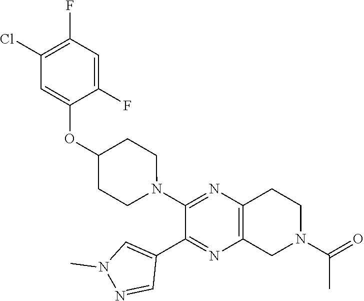 [SMILES: CC(=O)N1CCc2nc(N3CCC(Oc4cc(Cl)c(F)cc4F)CC3)c(-c3cnn(C)c3)nc2C1] | 1-{2-[4-(5-chloro-2,4- difluorophenoxy)piperidin- 1-yl]-3-(1-methyl-1H- pyrazol-4-yl)- 5H,6H,7H,8H-pyrido[3,4- b]pyrazin-6-yl}ethan-1- one | NA |
| 459 | 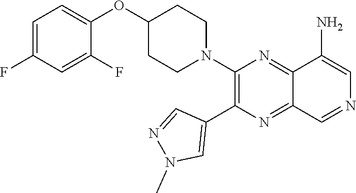 [SMILES: Cn1cc(-c2nc3cncc(N)c3nc2N2CCC(Oc3ccc(F)cc3F)CC2)cn1] | 2-[4-(2,4- difluorophenoxy)piperidin- 1-yl]-3-(1-methyl-1H- pyrazol-4-yl)pyrido[3,4- b]pyrazin-8-amine | NA |
| 460 | 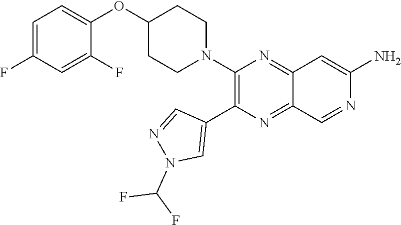 [SMILES: Nc1cc2nc(N3CCC(Oc4ccc(F)cc4F)CC3)c(-c3cnn(C(F)F)c3)nc2cn1] | 3-[1-(difluoromethyl)-1H- pyrazol-4-yl]-2-[4-(2,4- difluorophenoxy)piperidin- 1-yl]pyrido[3,4- b]pyrazin-7-amine | NA |
| 461 | 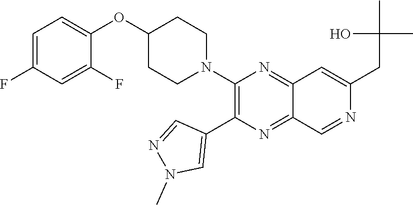 [SMILES: Cn1cc(-c2nc3cnc(CC(C)(C)O)cc3nc2N2CCC(Oc3ccc(F)cc3F)CC2)cn1] | 1-{2-[4-(2,4- difluorophenoxy)piperidin- 1-yl]-3-(1-methyl-1H- pyrazol-4-yl)pyrido[3,4- b]pyrazin-7-yl}-2- methylpropan-2-ol | NA |
| 462 | 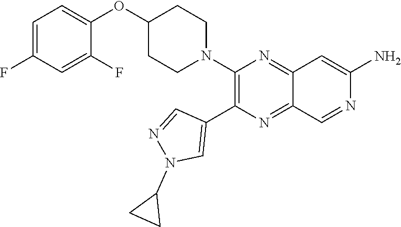 [SMILES: Nc1cc2nc(N3CCC(Oc4ccc(F)cc4F)CC3)c(-c3cnn(C4CC4)c3)nc2cn1] | 3-(1-cyclopropyl-1H- pyrazol-4-yl)-2-[4-(2,4- difluorophenoxy)piperidin- 1-yl]pyrido[3,4- b]pyrazin-7-amine | NA |
| 463 | 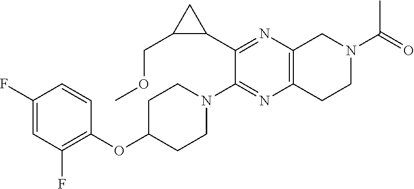 [SMILES: COCC1CC1c1nc2c(nc1N1CCC(Oc3ccc(F)cc3F)CC1)CCN(C(C)=O)C2] | 1-{2-[4-(2,4- difluorophenoxy)piperidin- 1-yl]-3-[2- (methoxymethyl)cyclopro pyl]-5H,6H,7H,8H- pyrido[3,4-b]pyrazin-6- yl}ethan-1-one | NA |
| 464 | 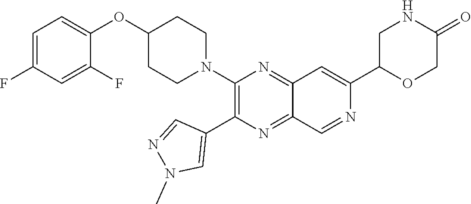 [SMILES: Cn1cc(-c2nc3cnc(C4CNC(=O)CO4)cc3nc2N2CCC(Oc3ccc(F)cc3F)CC2)cn1] | 6-{2-[4-(2,4- difluorophenoxy)piperidin- 1-yl]-3-(1-methyl-1H- pyrazol-4-yl)pyrido[3,4- b]pyrazin-7-yl}morpholin- 3-one | NA |
| 465 | 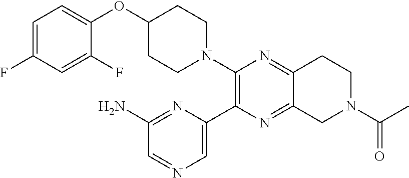 [SMILES: CC(=O)N1CCc2nc(N3CCC(Oc4ccc(F)cc4F)CC3)c(-c3cncc(N)n3)nc2C1] | 1-[3-(6-aminopyrazin-2- yl)-2-[4-(2,4- difluorophenoxy)piperidin- 1-yl]-5H,6H,7H,8H- pyrido[3,4-b]pyrazin-6- yl]ethan-1-one | NA |
| 466 | 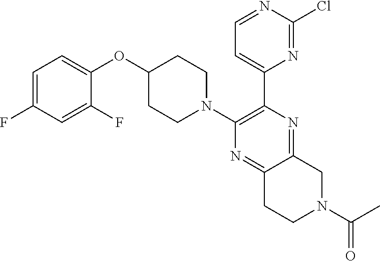 [SMILES: CC(=O)N1CCc2nc(N3CCC(Oc4ccc(F)cc4F)CC3)c(-c3ccnc(Cl)n3)nc2C1] | 1-[3-(2-chloropyrimidin- 4-yl)-2-[4-(2,4- difluorophenoxy)piperidin- 1-yl]-5H,6H,7H,8H- pyrido[3,4-b]pyrazin-6- yl]ethan-1-one | NA |
| 467 | 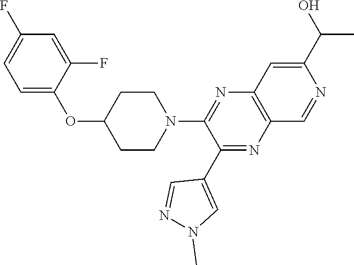 [SMILES: CC(O)c1cc2nc(N3CCC(Oc4ccc(F)cc4F)CC3)c(-c3cnn(C)c3)nc2cn1] | 1-{2-[4-(2,4- difluorophenoxy)piperidin- 1-yl]-3-(1-methyl-1H- pyrazol-4-yl)pyrido[3,4- b]pyrazin-7-yl}ethan-1-ol | NA |
| 468 | 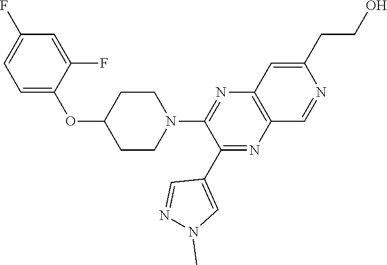 [SMILES: Cn1cc(-c2nc3cnc(CCO)cc3nc2N2CCC(Oc3ccc(F)cc3F)CC2)cn1] | 2-{2-[4-(2,4- difluorophenoxy)piperidin- 1-yl]-3-(1-methyl-1H- pyrazol-4-yl)pyrido[3,4- b]pyrazin-7-yl}ethan-1-ol | NA |
| 469 | 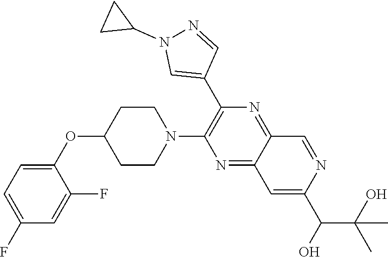 [SMILES: CC(C)(O)C(O)c1cc2nc(N3CCC(Oc4ccc(F)cc4F)CC3)c(-c3cnn(C4CC4)c3)nc2cn1] | 1-[3-(1-cyclopropyl-1H- pyrazol-4-yl)-2-[4-(2,4- difluorophenoxy)piperidin- 1-yl]pyrido[3,4- b]pyrazin-7-yl]-2- methylpropane-1,2-diol | NA |
| 470 | 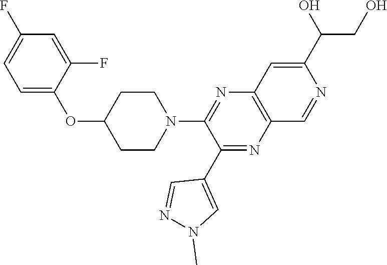 [SMILES: Cn1cc(-c2nc3cnc(C(O)CO)cc3nc2N2CCC(Oc3ccc(F)cc3F)CC2)cn1] | 1-{2-[4-(2,4- difluorophenoxy)piperidin- 1-yl]-3-(1-methyl-1H- pyrazol-4-yl)pyrido[3,4- b]pyrazin-7-yl}ethane-1,2- diol | NA |
| 471 | 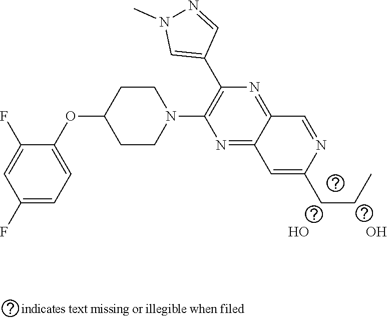 , or enantiomer | (1R,2S)-1-{2-[4-(2,4- difluorophenoxy)piperidin- 1-yl]-3-(1-methyl-1H- pyrazol-4-yl)pyrido[3,4- b]pyrazin-7-yl}propane- 1,2-diol, or enantiomer | NA |
| 472 | 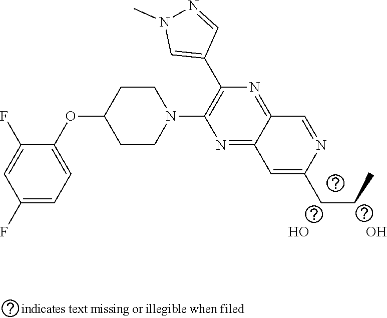 or enantiomer | (1S,2R)-1-{2-[4-(2,4- difluorophenoxy)piperidin- 1-yl]-3-(1-methyl-1H- pyrazol-4-yl)pyrido[3,4- b]pyrazin-7-yl}propane- 1,2-diol, or enantiomer | NA |
| 473 | 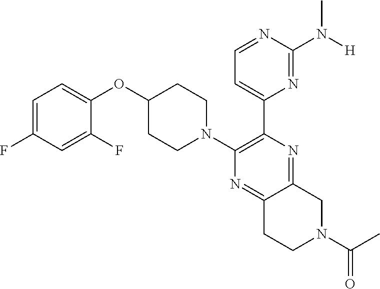 [SMILES: CNc1nccc(-c2nc3c(nc2N2CCC(Oc4ccc(F)cc4F)CC2)CCN(C(C)=O)C3)n1] | 1-{2-[4-(2,4- difluorophenoxy)piperidin- 1-yl]-3-[2- (methylamino)pyrimidin- 4-yl]-5H,6H,7H,8H- pyrido[3,4-b]pyrazin-6- yl}ethan-1-one | NA |
| 474 | 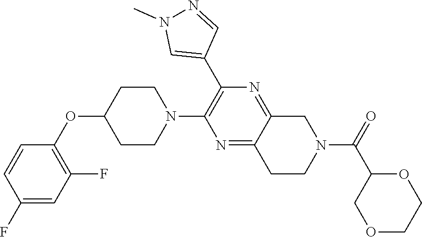 [SMILES: Cn1cc(-c2nc3c(nc2N2CCC(Oc4ccc(F)cc4F)CC2)CCN(C(=O)C2COCCO2)C3)cn1] | 4-(2,4-difluorophenoxy)- 1-[6-(1,4-dioxane-2- carbonyl)-3-(1-ethyl-1H- pyrazol-4-yl)- 5H,6H,7H,8H-pyrido[3,4- b]pyrazin-2-yl]piperidine | NA |
| 475 | 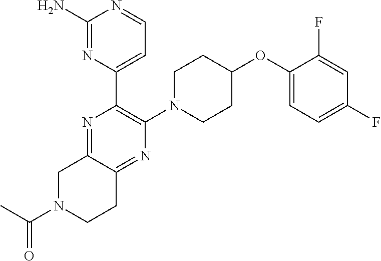 [SMILES: CC(=O)N1CCc2nc(N3CCC(Oc4ccc(F)cc4F)CC3)c(-c3ccnc(N)n3)nc2C1] | 1-[3-(2-aminopyrimidin- 4-yl)-2-[4-(2,4- difluorophenoxy)piperidin- 1-yl]-5H,6H,7H,8H- pyrido[3,4-b]pyrazin-6- yl]ethan-1-one | NA |
| 476 | 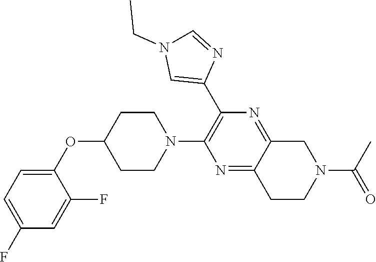 [SMILES: CCn1cnc(-c2nc3c(nc2N2CCC(Oc4ccc(F)cc4F)CC2)CCN(C(C)=O)C3)c1] | 1-{2-[4-(2,4- difluorophenoxy)piperidin- 1-yl]-3-(1-ethyl-1H- imidazol-4-yl)- 5H,6H,7H,8H-pyrido[3,4- b]pyrazin-6-yl}ethan-1- one | NA |
| 477 | 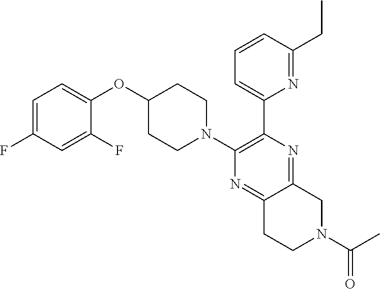 [SMILES: CCc1cccc(-c2nc3c(nc2N2CCC(Oc4ccc(F)cc4F)CC2)CCN(C(C)=O)C3)n1] | 1-{2-[4-(2,4- difluorophenoxy)piperidin- 1-yl]-3-(6-ethylpyridin-2- yl)-5H,6H,7H,8H- pyrido[3,4-b]pyrazin-6- yl}ethan-1-one | NA |
| 478 | 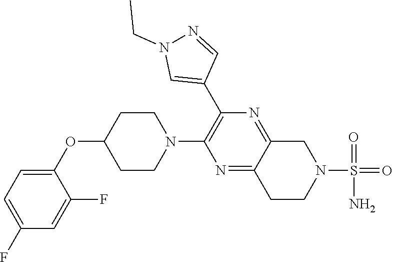 [SMILES: CCn1cc(-c2nc3c(nc2N2CCC(Oc4ccc(F)cc4F)CC2)CCN(S(N)(=O)=O)C3)cn1] | 2-[4-(2,4- difluorophenoxy)piperidin- 1-yl]-3-(1-ethyl-1H- pyrazol-4-yl)- 5H,6H,7H,8H-pyrido[3,4- b]pyrazine-6-sulfonamide | NA |
| 479 | 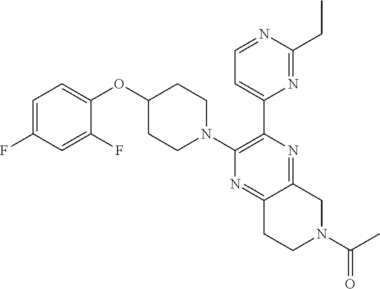 [SMILES: CCc1nccc(-c2nc3c(nc2N2CCC(Oc4ccc(F)cc4F)CC2)CCN(C(C)=O)C3)n1] | 1-{2-[4-(2,4- difluorophenoxy)piperidin- 1-yl]-3-(2- ethylpyrimidin-4-yl)- 5H,6H,7H,8H-pyrido[3,4- b]pyrazin-6-yl}ethan-1- one | NA |
| 480 | 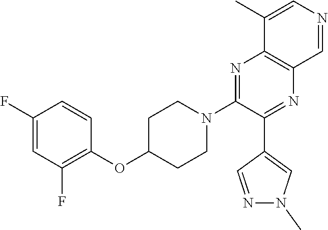 [SMILES: Cc1cncc2nc(-c3cnn(C)c3)c(N3CCC(Oc4ccc(F)cc4F)CC3)nc12] | 4-(2,4-difluorophenoxy)- 1-[8-methyl-3-(1-methyl- 1H-pyrazol-4- yl)pyrido[3,4-b]pyrazin-2- yl]piperidine | NA |
| 481 | 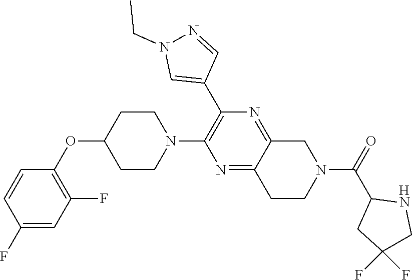 | 4-(2,4-difluorophenoxy)- 1-[6-(4,4- difluoropyrrolidine-2- carbonyl)-3-(1-ethyl-1H- pyrazol-4-yl)- 5H,6H,7H,8H-pyrido[3,4- b]pyrazin-2-yl]piperidine | NA |
| 482 | 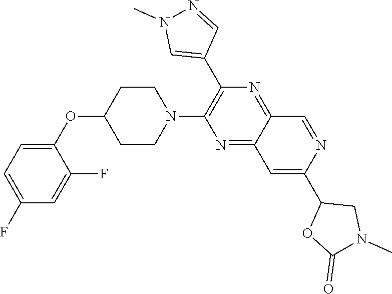 [SMILES: CN1CC(c2cc3nc(N4CCC(Oc5ccc(F)cc5F)CC4)c(-c4cnn(C)c4)nc3cn2)OC1=O] | 5-{2-[4-(2,4- difluorophenoxy)piperidin- 1-yl]-3-(1-methyl-1H- pyrazol-4-yl)pyrido[3,4- b]pyrazin-7-yl}-3-methyl- 1,3-oxazolidin-2-one | NA |
| 483 | 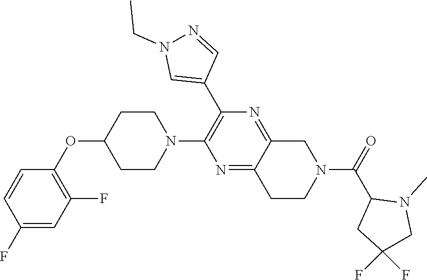 [SMILES: CCn1cc(-c2nc3c(nc2N2CCC(Oc4ccc(F)cc4F)CC2)CCN(C(=O)C2CC(F)(F)CN2C)C3)cn1] | 1-[6-(4,4-difluoro-1- methylpyrrolidine-2- carbonyl)-3-(1-ethyl-1H- pyrazol-4-yl)- 5H,6H,7H,8H-pyrido[3,4- b]pyrazin-2-yl]-4-(2,4- difluorophenoxy)piperidine | NA |
| 484 | 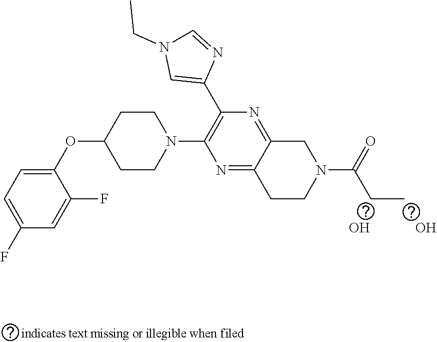 | 1-{2-[4-(2,4- difluorophenoxy)piperidin- 1-yl]-3-(1-ethyl-1H- pyrazol-4-yl)- 5H,6H,7H,8H-pyrido[3,4- b]pyrazin-6-yl}-2,3- dihydroxypropan-1-one | NA |
| 485 | 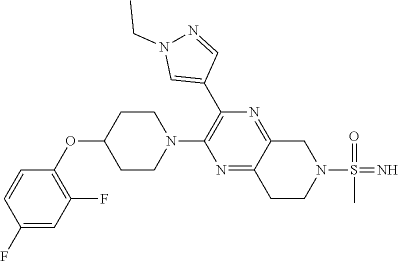 [SMILES: CCn1cc(-c2nc3c(nc2N2CCC(Oc4ccc(F)cc4F)CC2)CCN(S(C)(=N)=O)C3)cn1] | {2-[4-(2,4- difluorophenoxy)piperidin- 1-yl]-3-(1-ethyl-1H- pyrazol-4-yl)- 5H,6H,7H,8H-pyrido[3,4- b]pyrazin-6- yl}(imino)methyl-λ⁶- sulfanone | NA |
| 486 | 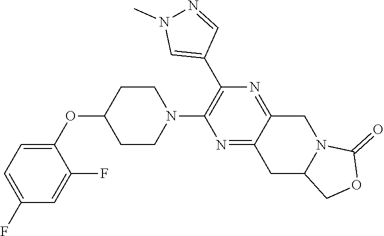 [SMILES: Cn1cc(-c2nc3c(nc2N2CCC(Oc4ccc(F)cc4F)CC2)CC2COC(=O)N2C3)cn1] | 11-[4-(2,4- difluorophenoxy)piperidin- 1-yl]-12-(1-methyl-1H- pyrazol-4-yl)-5-oxa- 3,10,13- triazatricyclo[7.4.0.0^{3,7}] trideca-1(13),9,11-trien-4- one | NA |
| 487 | 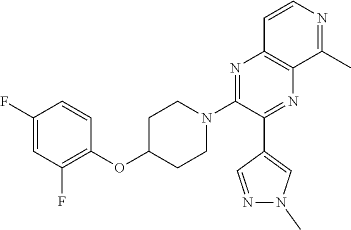 [SMILES: Cc1nccc2nc(N3CCC(Oc4ccc(F)cc4F)CC3)c(-c3cnn(C)c3)nc12] | 4-(2,4-difluorophenoxy)- 1-[5-methyl-3-(1-methyl- 1H-pyrazol-4- yl)pyrido[3,4-b]pyrazin-2- yl]piperidine | NA |
| 488 | 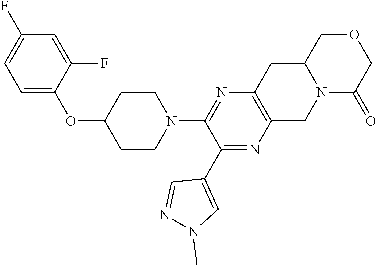 [SMILES: Cn1cc(-c2nc3c(nc2N2CCC(Oc4ccc(F)cc4F)CC2)CC2COCC(=O)N2C3)cn1] | 6-[4-(2,4- difluorophenoxy)piperidin- 1-yl]-5-(1-methyl-1H- pyrazol-4-yl)-12-oxa- 1,4,7- triazatricyclo[8.4.0.0^{3,8}]tet radeca-3(8),4,6-trien-14- one | NA |
| 489 | 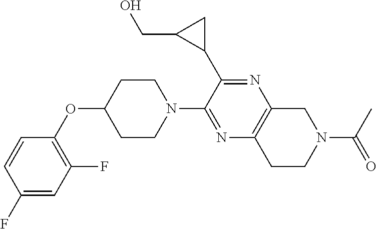 [SMILES: CC(=O)N1CCc2nc(N3CCC(Oc4ccc(F)cc4F)CC3)c(C3CC3CO)nc2C1] | 1-{2-[4-(2,4- difluorophenoxy)piperidin- 1-yl]-3-[2- (hydroxymethyl) cyclopropyl]-5H,6H, 7H,8H- pyrido[3,4-b]pyrazin-6- yl}ethan-1-one | NA |
| 490 | 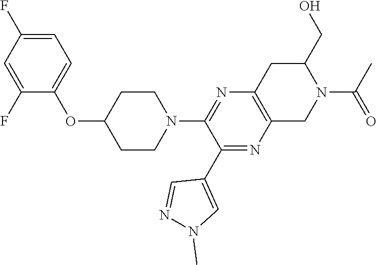 [SMILES: CC(=O)N1Cc2nc(-c3cnn(C)c3)c(N3CCC(Oc4ccc(F)cc4F)CC3)nc2CC1CO] | 1-{2-[4-(2,4- difluorophenoxy)piperidin- 1-yl]-7-(hydroxymethyl)- 3-(1-methyl-1H-pyrazol-4- yl)-5H,6H,7H,8H- pyrido[3,4-b]pyrazin-6- yl}ethan-1-one | NA |
| 491 | 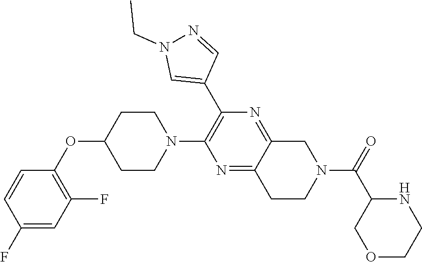 [SMILES: CCn1cc(-c2nc3c(nc2N2CCC(Oc4ccc(F)cc4F)CC2)CCN(C(=O)C2COCCN2)C3)cn1] | 3-{2-[4-(2,4- difluorophenoxy)piperidin- 1-yl]-3-(1-ethyl-1H- pyrazol-4-yl)- 5H,6H,7H,8H-pyrido[3,4- b]pyrazine-6- carbonyl}morpholine | NA |
| 492 | 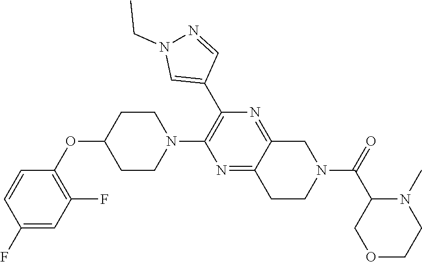 [SMILES: CCn1cc(-c2nc3c(nc2N2CCC(Oc4ccc(F)cc4F)CC2)CCN(C(=O)C2COCCN2C)C3)cn1] | 3-{2-[4-(2,4- difluorophenoxy)piperidin- 1-yl]-3-(1-ethyl-1H- pyrazol-4-yl)- 5H,6H,7H,8H-pyrido[3,4- b]pyrazine-6-carbonyl}-4- methylmorpholine | NA |
| 493 | 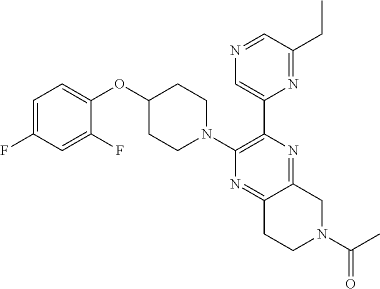 [SMILES: CCc1cncc(-c2nc3c(nc2N2CCC(Oc4ccc(F)cc4F)CC2)CCN(C(C)=O)C3)n1] | 1-{2-[4-(2,4- difluorophenoxy)piperidin- 1-yl]-3-(6-ethylpyrazin-2- yl)-5H,6H,7H,8H- pyrido[3,4-b]pyrazin-6- yl}ethan-1-one | NA |
| 494 | 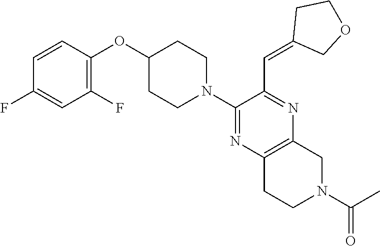 [SMILES: CC(=O)N1CCc2nc(N3CCC(Oc4ccc(F)cc4F)CC3)c(/C=C3/CCOC3)nc2C1] | 1-{2-[4-(2,4- difluorophenoxy)piperidin- 1-yl]-3-{[(3Z)-oxolan-3- ylidene]methyl}- 5H,6H,7H,8H-pyrido[3,4- b]pyrazin-6-yl}ethan-1- one | NA |
| 495 | 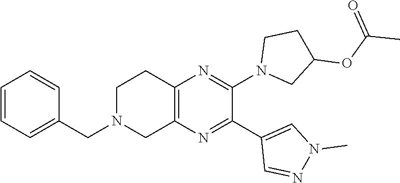 [SMILES: CC(=O)OC1CCN(c2nc3c(nc2-c2cnn(C)c2)CN(Cc2ccccc2)CC3)C1] | 1-[6-benzyl-3-(1-methyl-4- pyrazolyl)-5,6,7,8-tetrahydro- 1,4,6-triaza-2-naphthyl]-3- pyrrolidinyl acetate | NA |
| 496 | 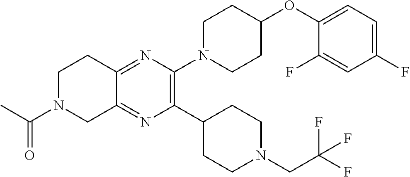 [SMILES: CC(=O)N1CCc2nc(N3CCC(Oc4ccc(F)cc4F)CC3)c(C3CCN(CC(F)(F)F)CC3)nc2C1] | 1-{2-[4-(2,4-difluorophenoxy)-1- piperidyl]-3-[1-(2,2,2- trifluoroethyl)-4-piperidyl]-5,6,7,8- tetrahydro-1,4,6-triaza-6- naphthyl}-1-ethanone | NA |
| 497 | 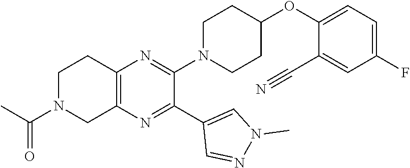 [SMILES: CC(=O)N1CCc2nc(N3CCC(Oc4ccc(F)cc4C#N)CC3)c(-c3cnn(C)c3)nc2C1] | 2-{1-[6-acetyl-3-(1-methyl-4- pyrazolyl)-5,6,7,8-tetrahydro- 1,4,6-triaza-2-naphthyl]-4- piperidyloxy}-5-fluorobenzonitrile | NA |
| 498 | 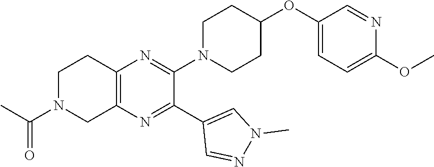 [SMILES: COc1ccc(OC2CCN(c3nc4c(nc3-c3cnn(C)c3)CN(C(C)=O)CC4)CC2)cn1] | 1-{2-[4-(6-methoxy-3-pyridyloxy)- 1-piperidyl]-3-(1-methyl-4- pyrazolyl)-5,6,7,8-tetrahydro- 1,4,6-triaza-6-naphthyl}-1- ethanone | NA |
| 499 | 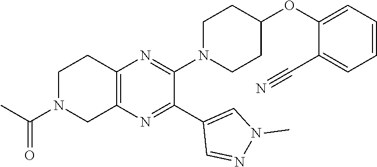 [SMILES: CC(=O)N1CCc2nc(N3CCC(Oc4ccccc4C#N)CC3)c(-c3cnn(C)c3)nc2C1] | o-{1-[6-acetyl-3-(1-methyl-4- pyrazolyl)-5,6,7,8-tetrahydro- 1,4,6-triaza-2-naphthyl]-4- piperidyloxy} benzonitrile | NA |
| 500 | 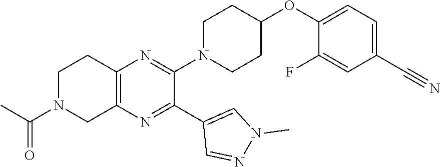 [SMILES: CC(=O)N1CCc2nc(N3CCC(Oc4ccc(C#N)cc4F)CC3)c(-c3cnn(C)c3)nc2C1] | 4-{1-[6-acetyl-3-(1-methyl-4- pyrazolyl)-5,6,7,8-tetrahydro- 1,4,6-triaza-2-naphthyl]-4- piperidyloxy}-3-fluorobenzonitrile | NA |
| 501 | 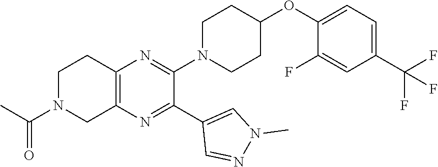 [SMILES: CC(=O)N1CCc2nc(N3CCC(Oc4ccc(C(F)(F)F)cc4F)CC3)c(-c3cnn(C)c3)nc2C1] | 1-(2-{4-[2-fluoro-4- (trifluoromethyl)phenoxy]-1- piperidyl}-3-(1-methyl-4- pyrazolyl)-5,6,7,8-tetrahydro- 1,4,6-triaza-6-naphthyl)-1- ethanone | NA |
| 502 | 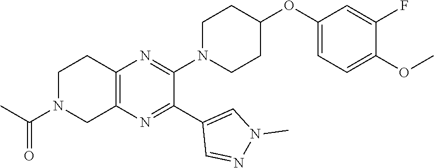 [SMILES: COc1ccc(OC2CCN(c3nc4c(nc3-c3cnn(C)c3)CN(C(C)=O)CC4)CC2)cc1F] | 1-{2-[4-(3-fluoro-4- methoxyphenoxy)-1-piperidyl]-3- (1-methyl-4-pyrazolyl)-5,6,7,8- tetrahydro-1,4,6-triaza-6- naphthyl}-1-ethanone | NA |
| 503 | 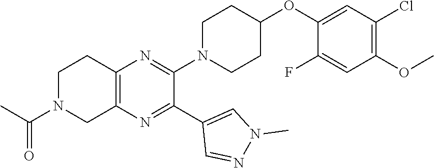 [SMILES: COc1cc(F)c(OC2CCN(c3nc4c(nc3-c3cnn(C)c3)CN(C(C)=O)CC4)CC2)cc1Cl] | 1-{2-[4-(5-chloro-2-fluoro-4- methoxyphenoxy)-1-piperidyl]-3- (1-methyl-4-pyrazolyl)-5,6,7,8- tetrahydro-1,4,6-triaza-6- naphthyl}-1-ethanone | NA |
| 504 | 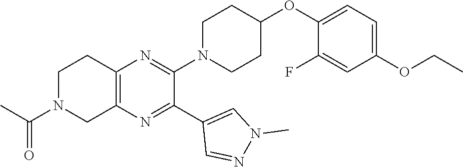 [SMILES: CCOc1ccc(OC2CCN(c3nc4c(nc3-c3cnn(C)c3)CN(C(C)=O)CC4)CC2)c(F)c1] | 1-{2-[4-(4-ethoxy-2- fluorophenoxy)-1-piperidyl]-3-(1- methyl-4-pyrazolyl)-5,6,7,8- tetrahydro-1,4,6-triaza-6- naphthyl}-1-ethanone | NA |
| 505 | 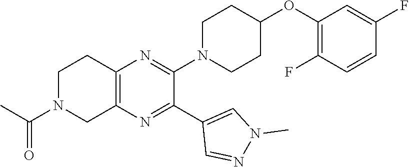 [SMILES: CC(=O)N1CCc2nc(N3CCC(Oc4cc(F)ccc4F)CC3)c(-c3cnn(C)c3)nc2C1] | 1-(2-{4-[(2,5- difluorophenyl)methyl]-1- piperazinyl}-3-(1-methyl-4- pyrazolyl)-5,6,7,8-tetrahydro- 1,4,6-triaza-6-naphthyl)-1- ethanone | NA |
| 506 | 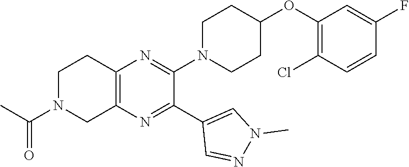 [SMILES: CC(=O)N1CCc2nc(N3CCC(Oc4cc(F)ccc4Cl)CC3)c(-c3cnn(C)c3)nc2C1] | 1-(2-{4-[(2-chloro-5- fluorophenyl)methyl]-1- piperazinyl}-3-(1-methyl-4- pyrazolyl)-5,6,7,8-tetrahydro- 1,4,6-triaza-6-naphthyl)-1- ethanone | NA |
| 507 | 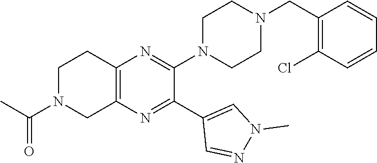 [SMILES: CC(=O)N1CCc2nc(N3CCN(Cc4ccccc4Cl)CC3)c(-c3cnn(C)c3)nc2C1] | 1-(2-{4-[(o-chlorophenyl)methyl]- 1-piperazinyl}-3-(1-methyl-4- pyrazolyl)-5,6,7,8-tetrahydro- 1,4,6-triaza-6-naphthyl)-1- ethanone | NA |
| 508 | 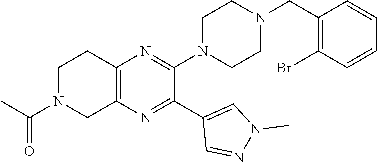 [SMILES: CC(=O)N1CCc2nc(N3CCN(Cc4ccccc4Br)CC3)c(-c3cnn(C)c3)nc2C1] | 1-(2-{4-[(o-bromophenyl)methyl]- 1-piperazinyl}-3-(1-methyl-4- pyrazolyl)-5,6,7,8-tetrahydro- 1,4,6-triaza-6-naphthyl)-1- ethanone | NA |
| 509 | 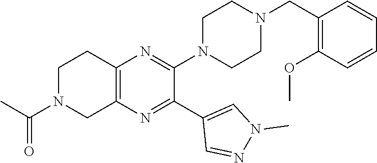 [SMILES: COc1ccccc1CN1CCN(c2nc3c(nc2-c2cnn(C)c2)CN(C(C)=O)CC3)CC1] | 1-(2-{4-[(o- methoxyphenyl)methyl]-1- piperazinyl}-3-(1-methyl-4- pyrazolyl)-5,6,7,8-tetrahydro- 1,4,6-triaza-6-naphthyl)-1- ethanone | NA |
| 510 | 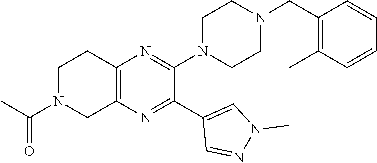 [SMILES: CC(=O)N1CCc2nc(N3CCN(Cc4ccccc4C)CC3)c(-c3cnn(C)c3)nc2C1] | 1-[3-(1-methyl-4-pyrazolyl)-2-{4- [(o-tolyl)methyl]-1-piperazinyl}- 5,6,7,8-tetrahydro-1,4,6-triaza-6- naphthyl]-1-ethanone | NA |
| 511 | 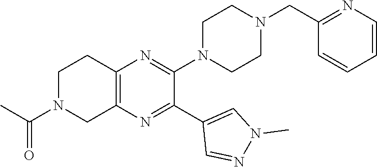 [SMILES: CC(=O)N1CCc2nc(N3CCN(Cc4ccccn4)CC3)c(-c3cnn(C)c3)nc2C1] | 1-[3-(1-methyl-4-pyrazolyl)-2-{4- [(2-pyridyl)methyl]-1- piperazinyl}-5,6,7,8-tetrahydro- 1,4,6-triaza-6-naphthyl]-1- ethanone | NA |
| 512 | 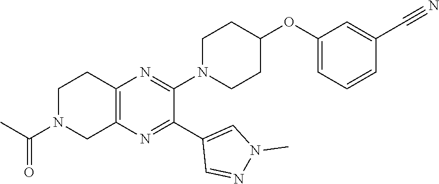 [SMILES: CC(=O)N1CCc2nc(N3CCC(Oc4cccc(C#N)c4)CC3)c(-c3cnn(C)c3)nc2C1] | m-({4-[6-acetyl-3-(1-methyl-4- pyrazolyl)-5,6,7,8-tetrahydro- 1,4,6-triaza-2-naphthyl]-1- piperazinyl}methyl)benzonitrile | NA |
| 513 | 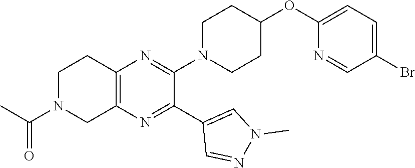 [SMILES: CC(=O)N1CCc2nc(N3CCC(Oc4ccc(Br)cn4)CC3)c(-c3cnn(C)c3)nc2C1] | 1-{2-[4-(5-bromo-2-pyridyloxy)-1- piperidyl]-3-(1-methyl-4- pyrazolyl)-5,6,7,8-tetrahydro- 1,4,6-triaza-6-naphthyl}-1- ethanone | NA |
| 514 | 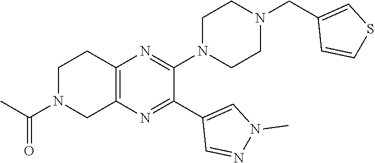 [SMILES: CC(=O)N1CCc2nc(N3CCN(Cc4ccsc4)CC3)c(-c3cnn(C)c3)nc2C1] | 1-[3-(1-methyl-4-pyrazolyl)-2-{4- [(3-thienyl)methyl]-1-piperazinyl}- 5,6,7,8-tetrahydro-1,4,6-triaza-6- naphthyl]-1-ethanone | NA |
| 515 | 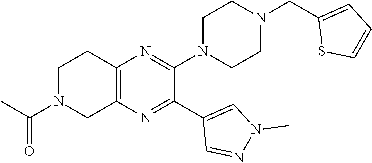 [SMILES: CC(=O)N1CCc2nc(N3CCN(Cc4cccs4)CC3)c(-c3cnn(C)c3)nc2C1] | 1-[3-(1-methyl-4-pyrazolyl)-2-(4- thenyl-1-piperazinyl)-5,6,7,8- tetrahydro-1,4,6-triaza-6- naphthyl]-1-ethanone | NA |
| 516 | 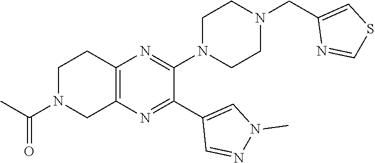 [SMILES: CC(=O)N1CCc2nc(N3CCN(Cc4cscn4)CC3)c(-c3cnn(C)c3)nc2C1] | 1-[3-(1-methyl-4-pyrazolyl)-2-{4- [(1,3-thiazol-4-yl)methyl]-1- piperazinyl}-5,6,7,8-tetrahydro- 1,4,6-triaza-6-naphthyl]-1- ethanone | NA |
| 517 | 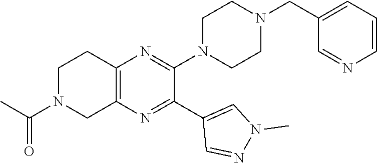 [SMILES: CC(=O)N1CCc2nc(N3CCN(Cc4cccnc4)CC3)c(-c3cnn(C)c3)nc2C1] | 1-[3-(1-methyl-4-pyrazolyl)-2-{4- [(3-pyridyl)methyl]-1- piperazinyl}-5,6,7,8-tetrahydro- 1,4,6-triaza-6-naphthyl]-1- ethanone | NA |
| 518 | 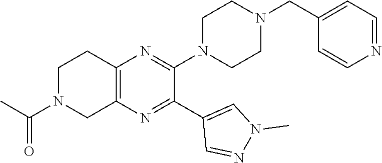 [SMILES: CC(=O)N1CCc2nc(N3CCN(Cc4ccncc4)CC3)c(-c3cnn(C)c3)nc2C1] | 1-[3-(1-methyl-4-pyrazolyl)-2-{4- [(4-pyridyl)methyl]-1- piperazinyl}-5,6,7,8-tetrahydro- 1,4,6-triaza-6-naphthyl]-1- ethanone | NA |
| 519 | 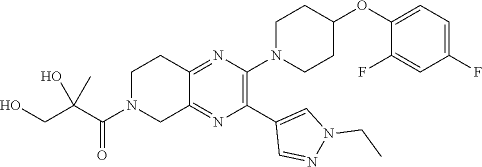 [SMILES: CCn1cc(-c2nc3c(nc2N2CCC(Oc4ccc(F)cc4F)CC2)CCN(C(=O)C(C)(O)CO)C3)cn1] | 1-{2-[4-(2,4-difluorophenoxy)-1- piperidyl]-3-(1-ethyl-4-pyrazolyl)- 5,6,7,8-tetrahydro-1,4,6-triaza-6- naphthyl}-2,3-dihydroxy-2-methyl- 1-propanone | NA |
| 520 | 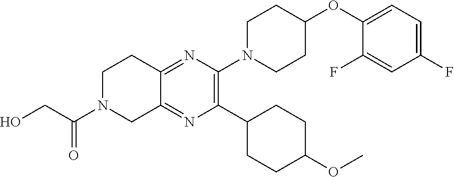 [SMILES: COC1CCC(c2nc3c(nc2N2CCC(Oc4ccc(F)cc4F)CC2)CCN(C(=O)CO)C3)CC1] | 1-{2-[4-(2,4-difluorophenoxy)-1- piperidyl]-3-(4- methoxycyclohexyl)-5,6,7,8- tetrahydro-1,4,6-triaza-6- naphthyl}-2-hydroxy-1-ethanone | NA |
| 521 | 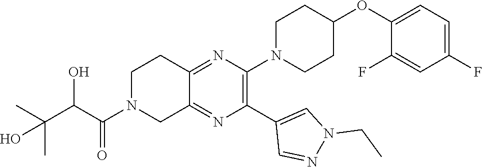 [SMILES: CCn1cc(-c2nc3c(nc2N2CCC(Oc4ccc(F)cc4F)CC2)CCN(C(=O)C(O)C(C)(C)O)C3)cn1] | 1-{2-[4-(2,4-difluorophenoxy)-1- piperidyl]-3-(1-ethyl-4-pyrazolyl)- 5,6,7,8-tetrahydro-1,4,6-triaza-6- naphthyl}-2,3-dihydroxy-3-methyl- 1-butanone | NA |
| 522 | 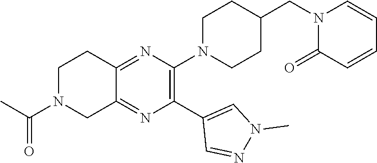 [SMILES: CC(=O)N1CCc2nc(N3CCC(Cn4ccccc4=O)CC3)c(-c3cnn(C)c3)nc2C1] | 1-({1-[6-acetyl-3-(1-methyl-4- pyrazolyl)-5,6,7,8-tetrahydro- 1,4,6-triaza-2-naphthyl]-4- piperidyl}methyl)-2(1H)- pyridinone | NA |
| 523 | 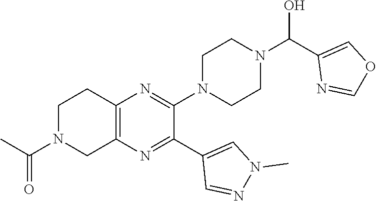 [SMILES: CC(=O)N1CCc2nc(N3CCN(C(O)c4cocn4)CC3)c(-c3cnn(C)c3)nc2C1] | 1-(2-{4-[hydroxy(1,3-oxazol-4- yl)methyl]-1-piperazinyl}-3-(1- methyl-4-pyrazolyl)-5,6,7,8- tetrahydro-1,4,6-triaza-6- naphthyl)-1-ethanone | NA |
| 524 | 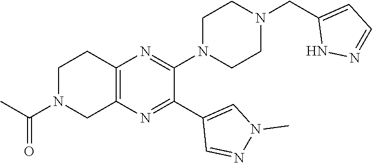 [SMILES: CC(=O)N1CCc2nc(N3CCN(Cc4ccn[nH]4)CC3)c(-c3cnn(C)c3)nc2C1] | 1-[3-(1-methyl-4-pyrazolyl)-2-{4- [(5-pyrazolyl)methyl]-1- piperazinyl}-5,6,7,8-tetrahydro- 1,4,6-triaza-6-naphthyl]-1- ethanone | NA |
| 525 | 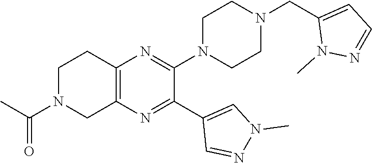 [SMILES: CC(=O)N1CCc2nc(N3CCN(Cc4ccnn4C)CC3)c(-c3cnn(C)c3)nc2C1] | 1-[3-(1-methyl-4-pyrazolyl)-2-{4- [(1-methyl-5-pyrazolyl)methyl]-1- piperazinyl}-5,6,7,8-tetrahydro- 1,4,6-triaza-6-naphthyl]-1- ethanone | NA |
| 526 | 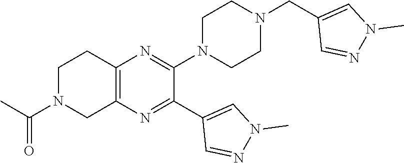 [SMILES: CC(=O)N1CCc2nc(N3CCN(Cc4cnn(C)c4)CC3)c(-c3cnn(C)c3)nc2C1] | 1-[3-(1-methyl-4-pyrazolyl)-2-{4- [(1-methyl-4-pyrazolyl)methyl]-1- piperazinyl}-5,6,7,8-tetrahydro- 1,4,6-triaza-6-naphthyl]-1- ethanone | NA |
| 527 | 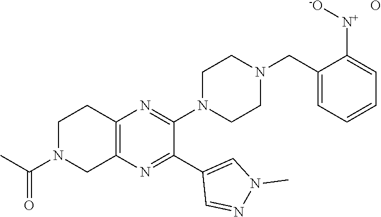 | 1-[3-(1-methyl-4-pyrazolyl)-2-{4- [(o-nitrophenyl)methyl]-1- piperazinyl}-5,6,7,8-tetrahydro- 1,4,6-triaza-6-naphthyl]-1- ethanone | NA |
| 528 | 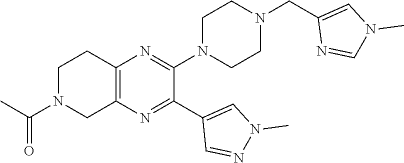 [SMILES: CC(=O)N1CCc2nc(N3CCN(Cc4cn(C)cn4)CC3)c(-c3cnn(C)c3)nc2C1] | 1-(2-{4-[(1-methyl-4- imidazolyl)methyl]-1-piperazinyl}- 3-(1-methyl-4-pyrazolyl)-5,6,7,8- tetrahydro-1,4,6-triaza-6- naphthyl)-1-ethanone | NA |
| 529 | 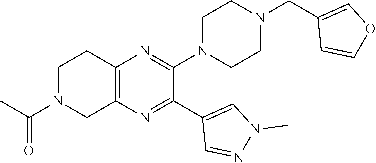 [SMILES: CC(=O)N1CCc2nc(N3CCN(Cc4ccoc4)CC3)c(-c3cnn(C)c3)nc2C1] | 1-(2-{4-[(3-furyl)methyl]-1- piperazinyl}-3-(1-methyl-4- pyrazolyl)-5,6,7,8-tetrahydro- 1,4,6-triaza-6-naphthyl)-1- ethanone | NA |
| 530 | 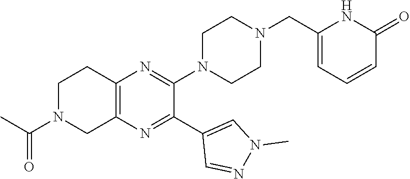 [SMILES: CC(=O)N1CCc2nc(N3CCN(Cc4cccc(=O)[nH]4)CC3)c(-c3cnn(C)c3)nc2C1] | 6-({4-[6-acetyl-3-(1-methyl-4- pyrazolyl)-5,6,7,8-tetrahydro- 1,4,6-triaza-2-naphthyl]-1- piperazinyl}methyl)-2(1H)- pyridinone | NA |
| 531 | 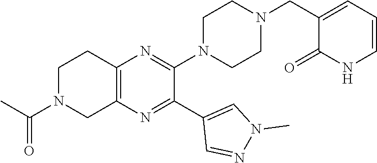 [SMILES: CC(=O)N1CCc2nc(N3CCN(Cc4ccc[nH]c4=O)CC3)c(-c3cnn(C)c3)nc2C1] | 3-({4-[6-acetyl-3-(1-methyl-4- pyrazolyl)-5,6,7,8-tetrahydro- 1,4,6-triaza-2-naphthyl]-1- piperazinyl}methyl)-2(1H)- pyridinone | NA |
| 532 | 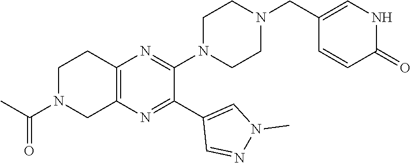 [SMILES: CC(=O)N1CCc2nc(N3CCN(Cc4ccc(=O)[nH]c4)CC3)c(-c3cnn(C)c3)nc2C1] | 5-({4-[6-acetyl-3-(1-methyl-4- pyrazolyl)-5,6,7,8-tetrahydro- 1,4,6-triaza-2-naphthyl]-1- piperazinyl}methyl)-2(1H)- pyridinone | NA |
| 533 | 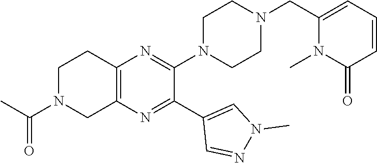 [SMILES: CC(=O)N1CCc2nc(N3CCN(Cc4cccc(=O)n4C)CC3)c(-c3cnn(C)c3)nc2C1] | 6-({4-[6-acetyl-3-(1-methyl-4- pyrazolyl)-5,6,7,8-tetrahydro- 1,4,6-triaza-2-naphthyl]-1- piperazinyl}methyl)-1-methyl- 2(1H)-pyridinone | NA |
| 534 | 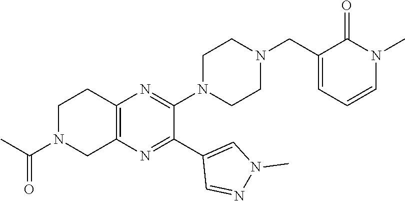 [SMILES: CC(=O)N1CCc2nc(N3CCN(Cc4cccn(C)c4=O)CC3)c(-c3cnn(C)c3)nc2C1] | 3-({4-[6-acetyl-3-(1-methyl-4- pyrazolyl)-5,6,7,8-tetrahydro- 1,4,6-triaza-2-naphthyl]-1- piperazinyl}methyl)-1-methyl- 2(1H)-pyridinone | NA |
| 535 | 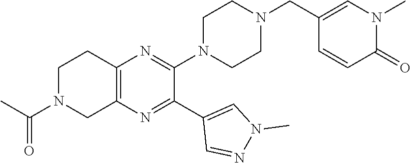 [SMILES: CC(=O)N1CCc2nc(N3CCN(Cc4ccc(=O)n(C)c4)CC3)c(-c3cnn(C)c3)nc2C1] | 5-({4-[6-acetyl-3-(1-methyl-4- pyrazolyl)-5,6,7,8-tetrahydro- 1,4,6-triaza-2-naphthyl]-1- piperazinyl}methyl)-1-methyl- 2(1H)-pyridinone | NA |
| 536 | 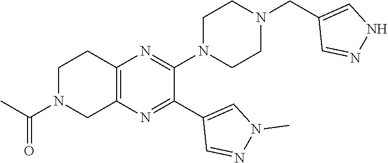 [SMILES: CC(=O)N1CCc2nc(N3CCN(Cc4cn[nH]c4)CC3)c(-c3cnn(C)c3)nc2C1] | 1-[3-(1-methyl-4-pyrazolyl)-2-{4- [(4-pyrazolyl)methyl]-1- piperazinyl}-5,6,7,8-tetrahydro- 1,4,6-triaza-6-naphthyl]-1- ethanone | NA |
| 537 | 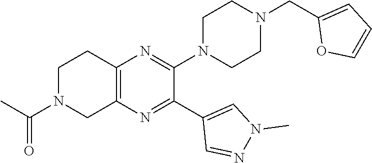 [SMILES: CC(=O)N1CCc2nc(N3CCN(Cc4ccco4)CC3)c(-c3cnn(C)c3)nc2C1] | 1-[2-(4-furfuryl-1-piperazinyl)-3- (1-methyl-4-pyrazolyl)-5,6,7,8- tetrahydro-1,4,6-triaza-6- naphthyl]-1-ethanone | NA |
| 538 | 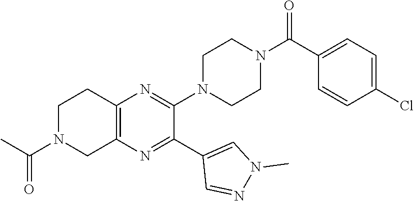 [SMILES: CC(=O)N1CCc2nc(N3CCN(C(=O)c4ccc(Cl)cc4)CC3)c(-c3cnn(C)c3)nc2C1] | 1-{2-[4-(p-chlorobenzoyl)-1- piperazinyl]-3-(1-methyl-4- pyrazolyl)-5,6,7,8-tetrahydro- 1,4,6-triaza-6-naphthyl}-1- ethanone | NA |
| 539 | 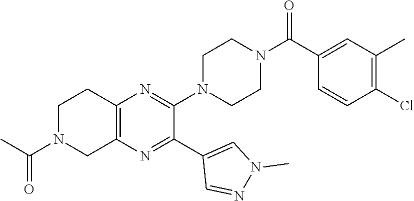 [SMILES: CC(=O)N1CCc2nc(N3CCN(C(=O)c4ccc(Cl)c(C)c4)CC3)c(-c3cnn(C)c3)nc2C1] | 1-{2-[4-(4-chloro-3-toluoyl)-1- piperazinyl]-3-(1-methyl-4- pyrazolyl)-5,6,7,8-tetrahydro- 1,4,6-triaza-6-naphthyl}-1- ethanone | NA |
| 540 | 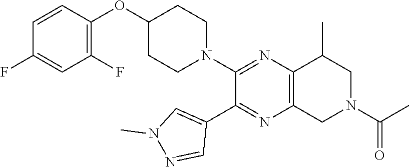 [SMILES: CC(=O)N1Cc2nc(-c3cnn(C)c3)c(N3CCC(Oc4ccc(F)cc4F)CC3)nc2C(C)C1] | 1-{2-[4-(2,4-difluorophenoxy)-1- piperidyl]-8-methyl-3-(1-methyl-4- pyrazolyl)-5,6,7,8-tetrahydro- 1,4,6-triaza-6-naphthyl}-1- ethanone | NA |
| 541 | 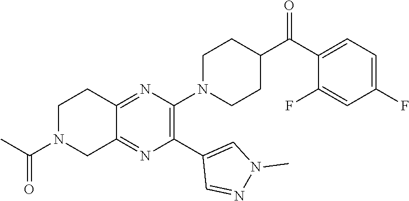 [SMILES: CC(=O)N1CCc2nc(N3CCC(C(=O)c4ccc(F)cc4F)CC3)c(-c3cnn(C)c3)nc2C1] | 1-{2-[4-(2,4-difluorobenzoyl)-1- piperidyl]-3-(1-methyl-4- pyrazolyl)-5,6,7,8-tetrahydro- 1,4,6-triaza-6-naphthyl}-1- ethanone | NA |
| 542 | 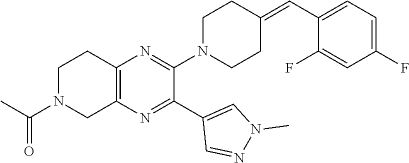 [SMILES: CC(=O)N1CCc2nc(N3CCC(=Cc4ccc(F)cc4F)CC3)c(-c3cnn(C)c3)nc2C1] | 1-(2-{4-[(2,4- difluorophenyl)methylene]-1- piperidyl}-3-(1-methyl-4- pyrazolyl)-5,6,7,8-tetrahydro- 1,4,6-triaza-6-naphthyl)-1- ethanone | NA |
| 543 | 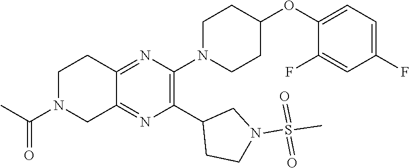 [SMILES: CC(=O)N1CCc2nc(N3CCC(Oc4ccc(F)cc4F)CC3)c(C3CCN(S(C)(=O)=O)C3)nc2C1] | 1-{2-[4-(2,4-difluorophenoxy)-1- piperidyl]-3-(1-mesyl-3- pyrrolidinyl)-5,6,7,8-tetrahydro- 1,4,6-triaza-6-naphthyl}-1- ethanone | NA |
| 544 | 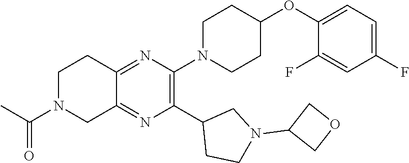 [SMILES: CC(=O)N1CCc2nc(N3CCC(Oc4ccc(F)cc4F)CC3)c(C3CCN(C4COC4)C3)nc2C1] | 1-{2-[4-(2,4-difluorophenoxy)-1- piperidyl]-3-[1-(3-oxetanyl)-3- pyrrolidinyl]-5,6,7,8-tetrahydro- 1,4,6-triaza-6-naphthyl}-1- ethanone | NA |
| 545 | 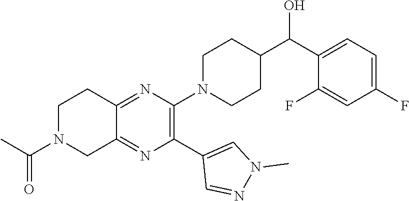 [SMILES: CC(=O)N1CCc2nc(N3CCC(C(O)c4ccc(F)cc4F)CC3)c(-c3cnn(C)c3)nc2C1] | 1-(2-{4-[(2,4- difluorophenyl)hydroxymethyl]-1- piperidyl}-3-(1-methyl-4- pyrazolyl)-5,6,7,8-tetrahydro- 1,4,6-triaza-6-naphthyl)-1- ethanone | NA |
| 546 | 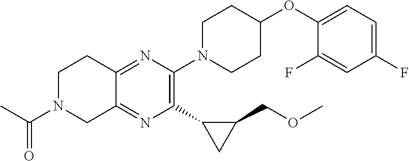 [SMILES: COC[C@H]1C[C@@H]1c1nc2c(nc1N1CCC(Oc3ccc(F)cc3F)CC1)CCN(C(C)=O)C2] | 1-{3-[(1S,2S)-2- (methoxymethyl)cyclopropyl]-2- [4-(2,4-difluorophenoxy)-1- piperidyl]-5,6,7,8-tetrahydro-1,4,6- triaza-6-naphthyl}-1-ethanone | not obtained |
| 547 | 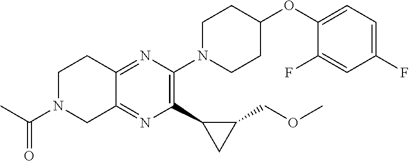 [SMILES: COC[C@@H]1C[C@H]1c1nc2c(nc1N1CCC(Oc3ccc(F)cc3F)CC1)CCN(C(C)=O)C2] | 1-{3-[(1R,2R)-2- (methoxymethyl)cyclopropyl]-2- [4-(2,4-difluorophenoxy)-1- piperidyl]-5,6,7,8-tetrahydro-1,4,6- triaza-6-naphthyl}-1-ethanone | not obtained |
| 548 | 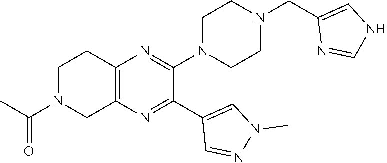 [SMILES: CC(=O)N1CCc2nc(N3CCN(Cc4c[nH]cn4)CC3)c(-c3cnn(C)c3)nc2C1] | 1-(2-{4-[(4-imidazolyl)methyl]-1- piperazinyl}-3-(1-methyl-4- pyrazolyl)-5,6,7,8-tetrahydro- 1,4,6-triaza-6-naphthyl)-1- ethanone | NA |
| 549 | 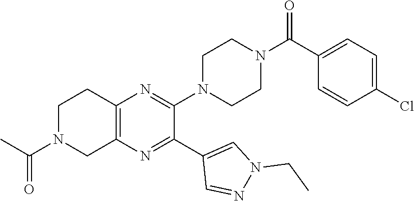 [SMILES: CCn1cc(-c2nc3c(nc2N2CCN(C(=O)c4ccc(Cl)cc4)CC2)CCN(C(C)=O)C3)cn1] | 1-{2-[4-(p-chlorobenzoyl)-1- piperazinyl]-3-(1-ethyl-4- pyrazolyl)-5,6,7,8-tetrahydro- 1,4,6-triaza-6-naphthyl}-1- ethanone | NA |
| 550 | 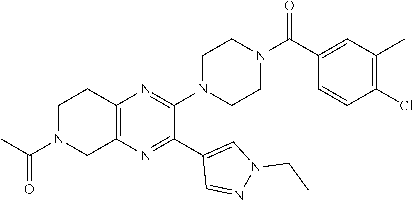 [SMILES: CCn1cc(-c2nc3c(nc2N2CCN(C(=O)c4ccc(Cl)c(C)c4)CC2)CCN(C(C)=O)C3)cn1] | 1-{2-[4-(4-chloro-3-toluoyl)-1- piperazinyl]-3-(1-ethyl-4- pyrazolyl)-5,6,7,8-tetrahydro- 1,4,6-triaza-6-naphthyl}-1- ethanone | NA |
| 551 | 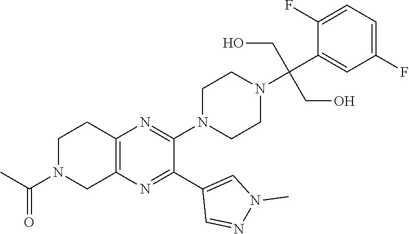 [SMILES: CC(=O)N1CCc2nc(N3CCN(C(CO)(CO)c4cc(F)ccc4F)CC3)c(-c3cnn(C)c3)nc2C1] | 1-(2-{4-[1-(2,5-difluorophenyl)-2- hydroxy-1-(hydroxymethyl)ethyl]- 1-piperazinyl}-3-(1-methyl-4- pyrazolyl)-5,6,7,8-tetrahydro- 1,4,6-triaza-6-naphthyl)-1- ethanone | NA |
| 552 | 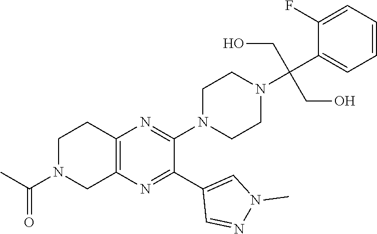 [SMILES: CC(=O)N1CCc2nc(N3CCN(C(CO)(CO)c4ccccc4F)CC3)c(-c3cnn(C)c3)nc2C1] | 1-(2-{4-[1-(o-fluorophenyl)-2- hydroxy-1-(hydroxymethyl)ethyl]- 1-piperazinyl}-3-(1-methyl-4- pyrazolyl)-5,6,7,8-tetrahydro- 1,4,6-triaza-6-naphthyl)-1- ethanone | NA |
| 553 | 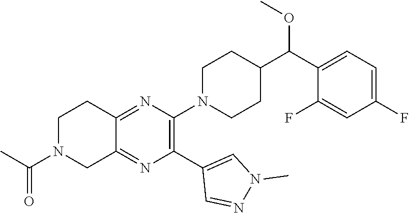 [SMILES: COC(c1ccc(F)cc1F)C1CCN(c2nc3c(nc2-c2cnn(C)c2)CN(C(C)=O)CC3)CC1] | 1-(2-{4-[(2,4- difluorophenyl)methoxymethyl]-1- piperidyl}-3-(1-methyl-4- pyrazolyl)-5,6,7,8-tetrahydro- 1,4,6-triaza-6-naphthyl)-1- ethanone | NA |
| 554 | 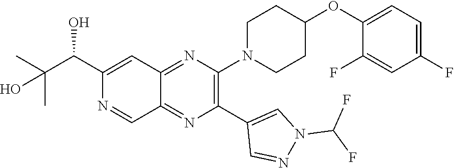 [SMILES: CC(C)(O)[C@H](O)c1cc2nc(N3CCC(Oc4ccc(F)cc4F)CC3)c(-c3cnn(C(F)F)c3)nc2cn1] or enantiomer | (R)-1-{3-[1-(difluoromethyl)-4- pyrazolyl]-2-[4-(2,4- difluorophenoxy)-1-piperidyl]- 1,4,6-triaza-7-naphthyl}-2-methyl- 1,2-propanediol, or enantiomer | (−) isomer |
| 555 | 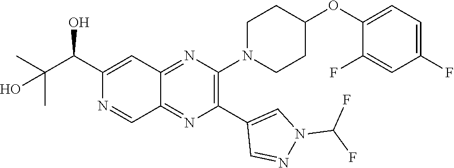 [SMILES: CC(C)(O)[C@@H](O)c1cc2nc(N3CCC(Oc4ccc(F)cc4F)CC3)c(-c3cnn(C(F)F)c3)nc2cn1] or enantiomer | (S)-1-{3-[1-(difluoromethyl)-4- pyrazolyl]-2-[4-(2,4- difluorophenoxy)-1-piperidyl]- 1,4,6-triaza-7-naphthyl}-2-methyl- 1,2-propanediol, or enantiomer | (+) isomer |
| 556 | 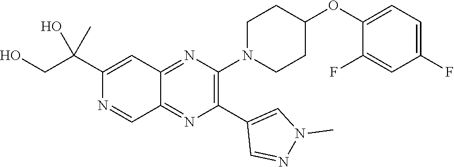 [SMILES: Cn1cc(-c2nc3cnc(C(C)(O)CO)cc3nc2N2CCC(Oc3ccc(F)cc3F)CC2)cn1] | 2-{2-[4-(2,4-difluorophenoxy)-1- piperidyl]-3-(1-methyl-4- pyrazolyl)-1,4,6-triaza-7- naphthyl}-1,2-propanediol | NA |
| 557 | 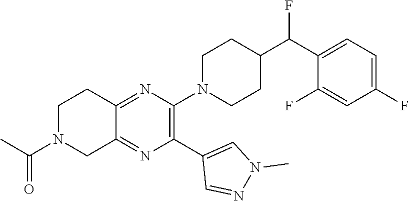 [SMILES: CC(=O)N1CCc2nc(N3CCC(C(F)c4ccc(F)cc4F)CC3)c(-c3cnn(C)c3)nc2C1] | 1-(2-{4-[(2,4- difluorophenyl)fluoromethyl]-1- piperidyl}-3-(1-methyl-4- pyrazolyl)-5,6,7,8-tetrahydro- 1,4,6-triaza-6-naphthyl)-1- ethanone | NA |
| 558 | 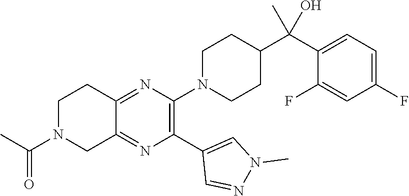 [SMILES: CC(=O)N1CCc2nc(N3CCC(C(C)(O)c4ccc(F)cc4F)CC3)c(-c3cnn(C)c3)nc2C1] | 1-(2-{4-[1-(2,4-difluorophenyl)-1- hydroxyethyl]-1-piperidyl}-3-(1- methyl-4-pyrazolyl)-5,6,7,8- tetrahydro-1,4,6-triaza-6- naphthyl)-1-ethanone | NA |
| 559 | 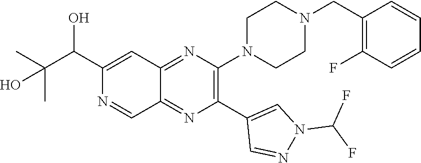 [SMILES: CC(C)(O)C(O)c1cc2nc(N3CCN(Cc4ccccc4F)CC3)c(-c3cnn(C(F)F)c3)nc2cn1] | 1-{3-[1-(difluoromethyl)-4- pyrazolyl]-2-{4-[(o- fluorophenyl)methyl]-1- piperazinyl}-1,4,6-triaza-7- naphthyl}-2-methyl-1,2- propanediol | NA |
| 560 | 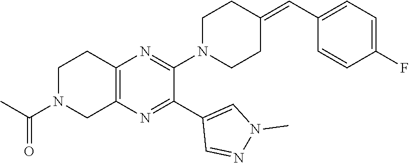 [SMILES: CC(=O)N1CCc2nc(N3CCC(=Cc4ccc(F)cc4)CC3)c(-c3cnn(C)c3)nc2C1] | 1-(2-{4-[(p- fluorophenyl)methylene]-1- piperidyl}-3-(1-methyl-4- pyrazolyl)-5,6,7,8-tetrahydro- 1,4,6-triaza-6-naphthyl)-1- ethanone | NA |
| 561 | 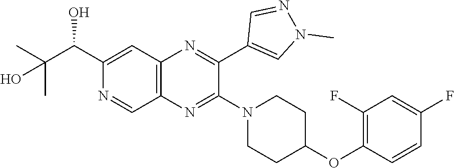 or enantiomer | (R)-1-{3-[4-(2,4-difluorophenoxy)- 1-piperidyl]-2-(1-methyl-4- pyrazolyl)-1,4,6-triaza-7- naphthyl}-2-methyl-1,2- propanediol, or enantiomer | (+) Isomer |
| 562 | 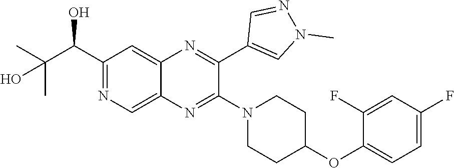 or enantiomer | (S)-1-{3-[4-(2,4-difluorophenoxy)- 1-piperidyl]-2-(1-methyl-4- pyrazolyl)-1,4,6-triaza-7- naphthyl}-2-methyl-1,2- propanediol, or enantiomer | (−) Isomer |
| 563 | 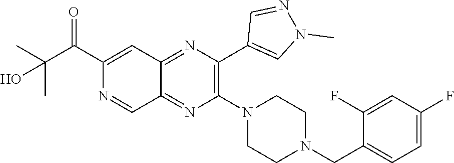 or enantiomer | 1-(3-{4-[(2,4- difluorophenyl)methyl]-1- piperazinyl}-2-(1-methyl-4- pyrazolyl)-1,4,6-triaza-7- naphthyl)-2-hydroxy-2-methyl-1- propanone | NA |
| 564 | 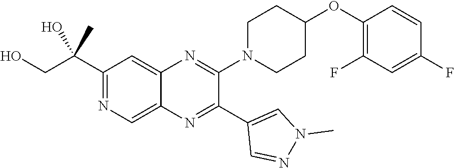 [SMILES: Cn1cc(-c2nc3cnc([C@@](C)(O)CO)cc3nc2N2CCC(Oc3ccc(F)cc3F)CC2)cn1] or enantiomer | (R)-2-{2-[4-(2,4-difluorophenoxy)- 1-piperidyl]-3-(1-methyl-4- pyrazolyl)-1,4,6-triaza-7- naphthyl}-1,2-propanediol, or enantiomer | (+) isomer |
| 565 | 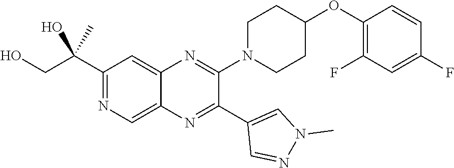 [SMILES: Cn1cc(-c2nc3cnc([C@](C)(O)CO)cc3nc2N2CCC(Oc3ccc(F)cc3F)CC2)cn1] or enantiomer | (S)-2-{2-[4-(2,4-difluorophenoxy)- 1-piperidyl]-3-(1-methyl-4- pyrazolyl)-1,4,6-triaza-7- naphthyl}-1,2-propanediol, or enantiomer | not obtained |
| 566 | 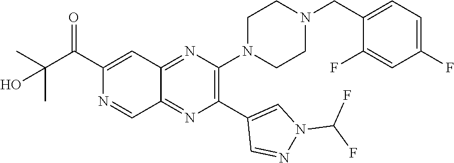 [SMILES: CC(C)(O)C(=O)c1cc2nc(N3CCN(Cc4ccc(F)cc4F)CC3)c(-c3cnn(C(F)F)c3)nc2cn1] | 1-{3-[1-(difluoromethyl)-4- pyrazolyl]-2-{4-[(2,4- difluorophenyl)methyl]-1- piperazinyl}-1,4,6-triaza-7- naphthyl}-2-hydroxy-2-methyl-1- propanone | NA |
| 567 | 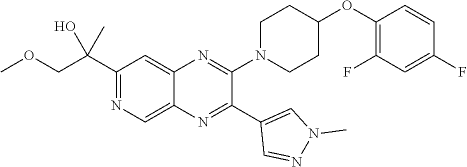 [SMILES: COCC(C)(O)c1cc2nc(N3CCC(Oc4ccc(F)cc4F)CC3)c(-c3cnn(C)c3)nc2cn1] | 2-{2-[4-(2,4-difluorophenoxy)-1- piperidyl]-3-(1-methyl-4- pyrazolyl)-1,4,6-triaza-7- naphthyl}-1-methoxy-2-propanol | NA |
| 568 | 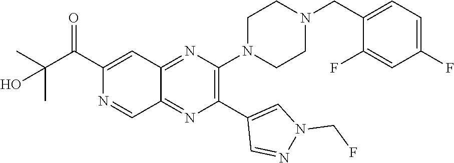 [SMILES: CC(C)(O)C(=O)c1cc2nc(N3CCN(Cc4ccc(F)cc4F)CC3)c(-c3cnn(CF)c3)nc2cn1] | 1-(2-{4-[(2,4- difluorophenyl)methyl]-1- piperazinyl}-3-[1-(fluoromethyl)- 4-pyrazolyl]-1,4,6-triaza-7- naphthyl)-2-hydroxy-2-methyl-1- propanone | NA |
| 569 | 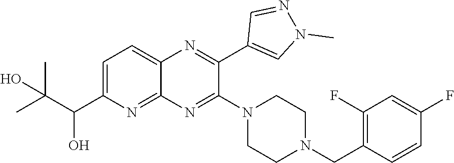 | 1-(3-{4-[(2,4- difluorophenyl)methyl]-1- piperazinyl}-2-(1-methyl-4- pyrazolyl)-1,4,5-triaza-6- naphthyl)-2-methyl-1,2- propanediol | NA |
| 570 | 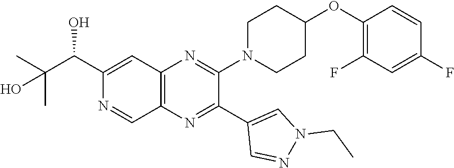 [SMILES: CCn1cc(-c2nc3cnc([C@@H](O)C(C)(C)O)cc3nc2N2CCC(Oc3ccc(F)cc3F)CC2)cn1] or enantiomer | (R)-1-{2-[4-(2,4-difluorophenoxy)- 1-piperidyl]-3-(1-ethyl-4- pyrazolyl)-1,4,6-triaza-7- naphthyl}-2-methyl-1,2- propanediol, or enantiomer | (+) Isomer |
| 571 | 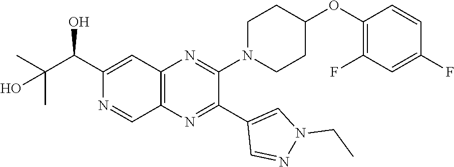 [SMILES: CCn1cc(-c2nc3cnc([C@H](O)C(C)(C)O)cc3nc2N2CCC(Oc3ccc(F)cc3F)CC2)cn1] or enantiomer | (S)-1-{2-[4-(2,4-difluorophenoxy)- 1-piperidyl]-3-(1-ethyl-4- pyrazolyl)-1,4,6-triaza-7- naphthyl}-2-methyl-1,2- propanediol, or enantiomer | (−) Isomer |
| 572 | 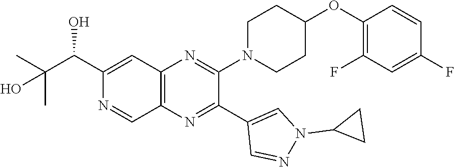 [SMILES: CC(C)(O)[C@H](O)c1cc2nc(N3CCC(Oc4ccc(F)cc4F)CC3)c(-c3cnn(C4CC4)c3)nc2cn1] or enantiomer | (R)-1-{3-(1-cyclopropyl-4- pyrazolyl)-2-[4-(2,4- difluorophenoxy)-1-piperidyl]- 1,4,6-triaza-7-naphthyl}-2-methyl- 1,2-propanediol, or enantiomer | (+) Isomer |
| 573 | 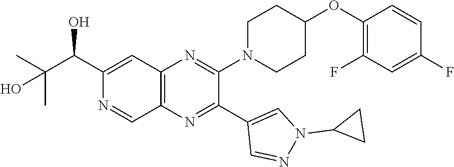 [SMILES: CC(C)(O)[C@@H](O)c1cc2nc(N3CCC(Oc4ccc(F)cc4F)CC3)c(-c3cnn(C4CC4)c3)nc2cn1] or enantiomer | (S)-1-{3-(1-cyclopropyl-4- pyrazolyl)-2-[4-(2,4- difluorophenoxy)-1-piperidyl]- 1,4,6-triaza-7-naphthyl}-2-methyl- 1,2-propanediol, or enantiomer | (−) isomer |
| 574 | 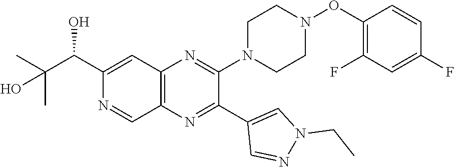 [SMILES: CCn1cc(-c2nc3cnc([C@@H](O)C(C)(C)O)cc3nc2N2CCN(Oc3ccc(F)cc3F)CC2)cn1] or enantiomer | (R)-1-(2-{4-[(2,4- difluorophenyl)methyl]-1- piperazinyl}-3-(1-ethyl-4- pyrazolyl)-1,4,6-triaza-7- naphthyl)-2-methyl-1,2- propanediol, or enantiomer | (+) Isomer |
| 575 | 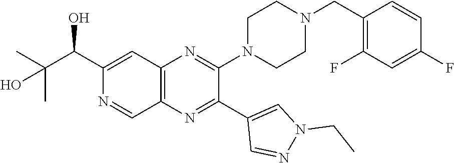 [SMILES: CCn1cc(-c2nc3cnc([C@H](O)C(C)(C)O)cc3nc2N2CCN(Cc3ccc(F)cc3F)CC2)cn1] or enantiomer | (S)-1-(2-{4-[(2,4- difluorophenyl)methyl]-1- piperazinyl}-3-(1-ethyl-4- pyrazolyl)-1,4,6-triaza-7- naphthyl)-2-methyl-1,2- propanediol, or enantiomer | (−) Isomer |
| 576 | 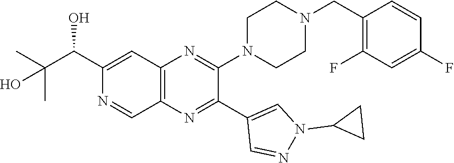 [SMILES: CC(C)(O)[C@H](O)c1cc2nc(N3CCN(Cc4ccc(F)cc4F)CC3)c(-c3cnn(C4CC4)c3)nc2cn1] or enantiomer | (R)-1-[3-(1-cyclopropyl-4- pyrazolyl)-2-{4-[(2,4- difluorophenyl)methyl]-1- piperazinyl}-1,4,6-triaza-7- naphthyl]-2-methyl-1,2- propanediol, or enantiomer | (+) Isomer |
| 577 | 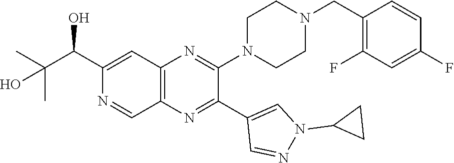 [SMILES: CC(C)(O)[C@@H](O)c1cc2nc(N3CCN(Cc4ccc(F)cc4F)CC3)c(-c3cnn(C4CC4)c3)nc2cn1] or enantiomer | (S)-1-[3-(1-cyclopropyl-4- pyrazolyl)-2-{4-[(2,4- difluorophenyl)methyl]-1- piperazinyl}-1,4,6-triaza-7- naphthyl]-2-methyl-1,2- propanediol, or enantiomer | (−) Isomer |
| 578 | 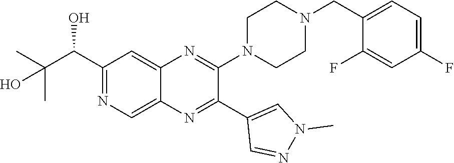 [SMILES: Cn1cc(-c2nc3cnc([C@@H](O)C(C)(C)O)cc3nc2N2CCN(Cc3ccc(F)cc3F)CC2)cn1] or enantiomer | (R)-1-(2-{4-[(2,4- difluorophenyl)methyl]-1- piperazinyl}-3-(1-methyl-4- pyrazolyl)-1,4,6-triaza-7- naphthyl)-2-methyl-1,2- propanediol, or enantiomer | (+) Isomer |
| 579 | 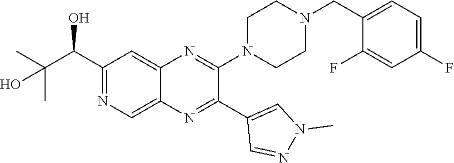 [SMILES: Cn1cc(-c2nc3cnc([C@H](O)C(C)(C)O)cc3nc2N2CCN(Cc3ccc(F)cc3F)CC2)cn1] or enantiomer | (S)-1-(2-{4-[(2,4- difluorophenyl)methyl]-1- piperazinyl}-3-(1-methyl-4- pyrazolyl)-1,4,6-triaza-7- naphthyl)-2-methyl-1,2- propanediol, or enantiomer | (−) Isomer |
| 580 | 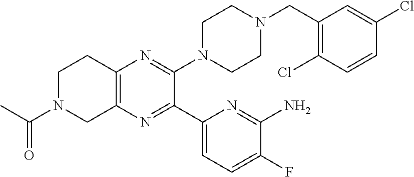 [SMILES: CC(=O)N1CCc2nc(N3CCN(Cc4cc(Cl)ccc4Cl)CC3)c(-c3ccc(F)c(N)n3)nc2C1] | 1-[3-(6-amino-5-fluoro-2-pyridyl)- 2-{4-[(2,5-dichlorophenyl)methyl]- 1-piperazinyl}-5,6,7,8-tetrahydro- 1,4,6-triaza-6-naphthyl]-1- ethanone | NA |
| 581 | 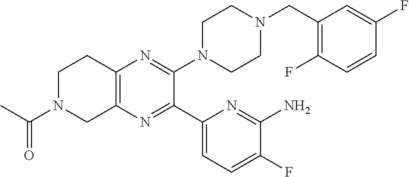 [SMILES: CC(=O)N1CCc2nc(N3CCN(Cc4cc(F)ccc4F)CC3)c(-c3ccc(F)c(N)n3)nc2C1] | 1-[3-(6-amino-5-fluoro-2-pyridyl)- 2-{4-[(2,5-difluorophenyl)methyl]- 1-piperazinyl}-5,6,7,8-tetrahydro- 1,4,6-triaza-6-naphthyl]-1- ethanone | NA |
| 582 | 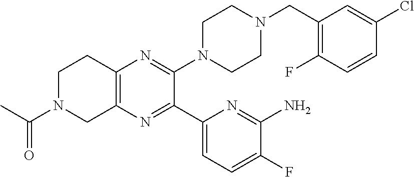 [SMILES: CC(=O)N1CCc2nc(N3CCN(Cc4cc(Cl)ccc4F)CC3)c(-c3ccc(F)c(N)n3)nc2C1] | 1-[3-(6-amino-5-fluoro-2-pyridyl)- 2-{4-[(5-chloro-2- fluorophenyl)methyl]-1- piperazinyl}-5,6,7,8-tetrahydro- 1,4,6-triaza-6-naphthyl]-1- ethanone | NA |
| 583 | 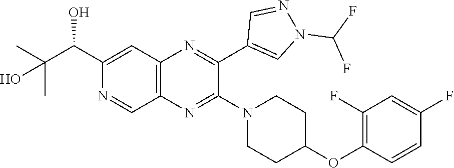 or enantiomer | (R)-1-{2-[1-(difluoromethyl)-4- pyrazolyl]-3-[4-(2,4- difluorophenoxy)-1-piperidyl]- 1,4,6-triaza-7-naphthyl}-2-methyl- 1,2-propanediol, or enantiomer | (+) Isomer |
| 584 | 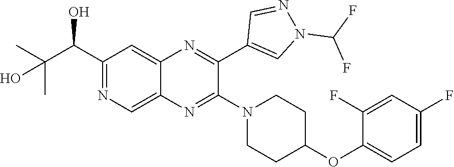 or enantiomer | (S)-1-{2-[1-(difluoromethyl)-4- pyrazolyl]-3-[4-(2,4- difluorophenoxy)-1-piperidyl]- 1,4,6-triaza-7-naphthyl}-2-methyl- 1,2-propanediol, or enantiomer | (−) Isomer |
| 585 | 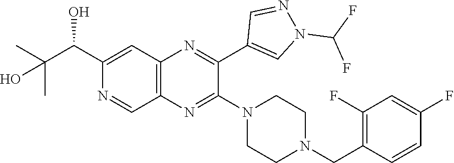 or enantiomer | (R)-1-{2-[1-(difluoromethyl)-4- pyrazolyl]-3-{4-[(2,4- difluorophenyl)methyl]-1- piperazinyl}-1,4,6-triaza-7- naphthyl}-2-methyl-1,2- propanediol, or enantiomer | (+) Isomer |
| 586 | 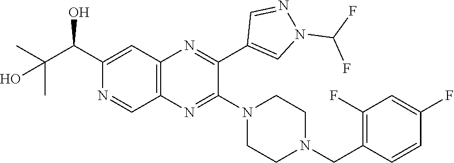 or enantiomer | (S)-1-{2-[1-(difluoromethyl)-4- pyrazolyl]-3-{4-[(2,4- difluorophenyl)methyl]-1- piperazinyl}-1,4,6-triaza-7- naphthyl}-2-methyl-1,2- propanediol, or enantiomer | (−) Isomer |
| 587 | 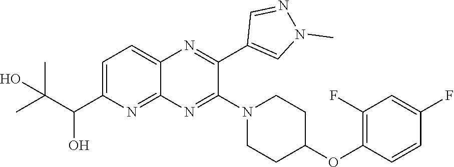 | 1-{3-[4-(2,4-difluorophenoxy)-1- piperidyl]-2-(1-methyl-4- pyrazolyl)-1,4,5-triaza-6- naphthyl}-2-methyl-1,2- propanediol | NA |
| 588 | 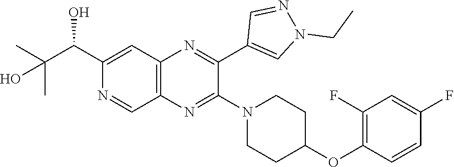 or enantiomer | (R)-1-{3-[4-(2,4-difluorophenoxy)- 1-piperidyl]-2-(1-ethyl-4- pyrazolyl)-1,4,6-triaza-7- naphthyl}-2-methyl-1,2- propanediol, or enantiomer | (+) Isomer |
| 589 | 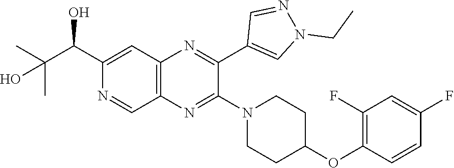 or enantiomer | (S)-1-{3-[4-(2,4-difluorophenoxy)- 1-piperidyl]-2-(1-ethyl-4- pyrazolyl)-1,4,6-triaza-7- naphthyl}-2-methyl-1,2- propanediol, or enantiomer | (−) Isomer |
| 590 | 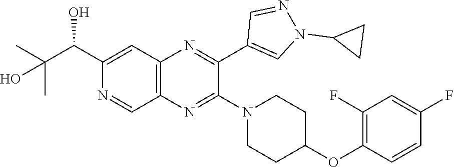 or enantiomer | (R)-1-{2-(1-cyclopropyl-4- pyrazolyl)-3-[4-(2,4- difluorophenoxy)-1-piperidyl]- 1,4,6-triaza-7-naphthyl}-2-methyl- 1,2-propanediol, or enantiomer | (+) Isomer |
| 591 | 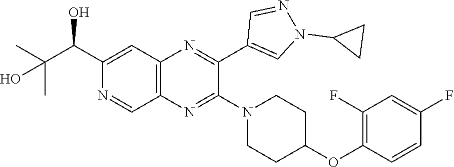 or enantiomer | (S)-1-{2-(1-cyclopropyl-4- pyrazolyl)-3-[4-(2,4- difluorophenoxy)-1-piperidyl]- 1,4,6-triaza-7-naphthyl}-2-methyl- 1,2-propanediol, or enantiomer | (−) Isomer |
| 592 | 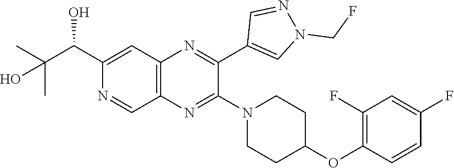 or enantiomer | (R)-1-{3-[4-(2,4-difluorophenoxy)- 1-piperidyl]-2-[1-(fluoromethyl)-4- pyrazolyl]-1,4,6-triaza-7- naphthyl}-2-methyl-1,2- propanediol, or enantiomer | (+) Isomer |
| 593 | 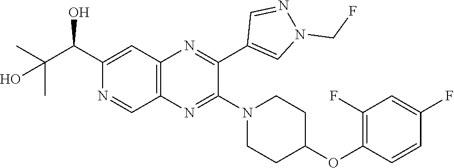 or enantiomer | (S)-1-{3-[4-(2,4-difluorophenoxy)- 1-piperidyl]-2-[1-(fluoromethyl)-4- pyrazolyl]-1,4,6-triaza-7- naphthyl}-2-methyl-1,2- propanediol, or enantiomer | (−) Isomer |
| 594 | 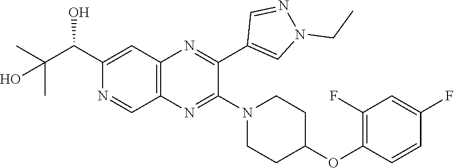 or enantiomer | (R)-1-(3-{4-[(2,4- difluorophenyl)methyl]-1- piperazinyl}-2-(1-ethyl-4- pyrazolyl)-1,4,6-triaza-7- naphthyl)-2-methyl-1,2- propanediol, or enantiomer | (+) Isomer |
| 595 | 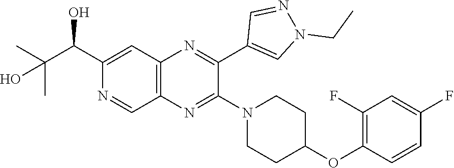 or enantiomer | (S)-1-(3-{4-[(2,4- difluorophenyl)methyl]-1- piperazinyl}-2-(1-ethyl-4- pyrazolyl)-1,4,6-triaza-7- naphthyl)-2-methyl-1,2- propanediol, or enantiomer | (−) isomer |
| 596 | 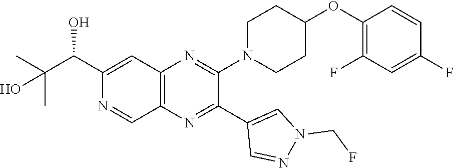 [SMILES: CC(C)(O)[C@H](O)c1cc2nc(N3CCC(Oc4ccc(F)cc4F)CC3)c(-c3cnn(CF)c3)nc2cn1] or enantiomer | (R)-1-{2-[4-(2,4-difluorophenoxy)- 1-piperidyl]-3-[1-(fluoromethyl)-4- pyrazolyl]-1,4,6-triaza-7- naphthyl}-2-methyl-1,2- propanediol, or enantiomer | (+) Isomer |
| 597 | 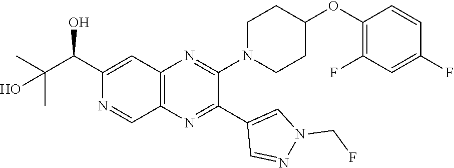 [SMILES: CC(C)(O)[C@@H](O)c1cc2nc(N3CCC(Oc4ccc(F)cc4F)CC3)c(-c3cnn(CF)c3)nc2cn1] or enantiomer | (S)-1-{2-[4-(2,4-difluorophenoxy)- 1-piperidyl]-3-[1-(fluoromethyl)-4- pyrazolyl]-1,4,6-triaza-7- naphthyl}-2-methyl-1,2- propanediol, or enantiomer | (−) Isomer |
| 598 | 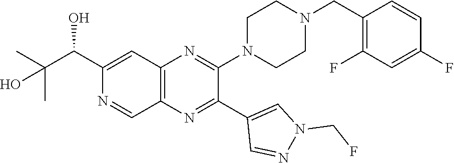 [SMILES: CC(C)(O)[C@H](O)c1cc2nc(N3CCN(Cc4ccc(F)cc4F)CC3)c(-c3cnn(CF)c3)nc2cn1] or enantiomer | (R)-1-(2-{4-[(2,4- difluorophenyl)methyl]-1- piperazinyl}-3-[1-(fluoromethyl)- 4-pyrazolyl]-1,4,6-triaza-7- naphthyl)-2-methyl-1,2- propanediol, or enantiomer | (+) Isomer |
| 599 | 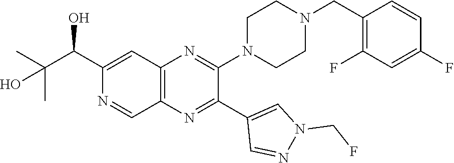 [SMILES: CC(C)(O)[C@@H](O)c1cc2nc(N3CCN(Cc4ccc(F)cc4F)CC3)c(-c3cnn(CF)c3)nc2cn1] or enantiomer | (S)-1-(2-{4-[(2,4- difluorophenyl)methyl]-1- piperazinyl}-3-[1-(fluoromethyl)- 4-pyrazolyl]-1,4,6-triaza-7- naphthyl)-2-methyl-1,2- propanediol, or enantiomer | (−) Isomer |
| 600 | 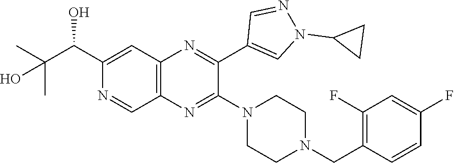 or enantiomer | (R)-1-[2-(1-cyclopropyl-4- pyrazolyl)-3-{4-[(2,4- difluorophenyl)methyl]-1- piperazinyl}-1,4,6-triaza-7- naphthyl]-2-methyl-1,2- propanediol, or enantiomer | (+) Isomer |
| 601 | 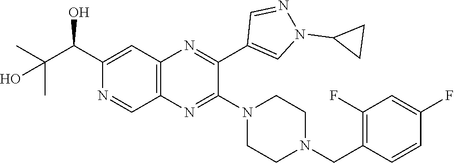 , or enantiomer | (S)-1-[2-(1-cyclopropyl-4- pyrazolyl)-3-{4-[(2,4- difluorophenyl)methyl]-1- piperazinyl}-1,4,6-triaza-7- naphthyl]-2-methyl-1,2- propanediol, or enantiomer | (−) Isomer |
| 602 | 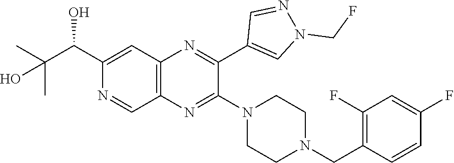 or enantiomer | (R)-1-(3-{4-[(2,4- difluorophenyl)methyl]-1- piperazinyl}-2-[1-(fluoromethyl)- 4-pyrazolyl]-1,4,6-triaza-7- naphthyl)-2-methyl-1,2- propanediol, or enantiomer | (+) Isomer |
| 603 | 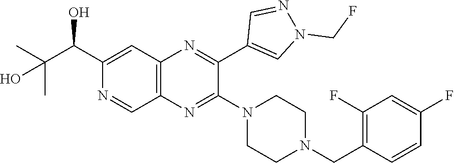 , or enantiomer | (S)-1-(3-{4-[(2,4- difluorophenyl)methyl]-1- piperazinyl}-2-[1-(fluoromethyl)- 4-pyrazolyl]-1,4,6-triaza-7- naphthyl)-2-methyl-1,2- propanediol, or enantiomer | (−) Isomer |
| 604 | 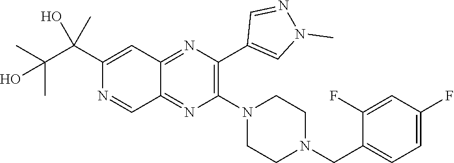 | 2-(3-{4-[(2,4- difluorophenyl)methyl]-1- piperazinyl}-2-(1-methyl-4- pyrazolyl)-1,4,6-triaza-7- naphthyl)-3-methyl-2,3-butanediol | NA |
| 605 | 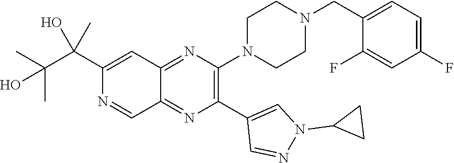 [SMILES: CC(C)(O)C(C)(O)c1cc2nc(N3CCN(Cc4ccc(F)cc4F)CC3)c(-c3cnn(C4CC4)c3)nc2cn1] | 2-[3-(1-cyclopropyl-4-pyrazolyl)- 2-{4-[(2,4-difluorophenyl)methyl]- 1-piperazinyl}-1,4,6-triaza-7- naphthyl]-3-methyl-2,3-butanediol | NA |
| 606 | 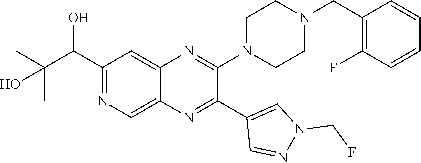 [SMILES: CC(C)(O)C(O)c1cc2nc(N3CCN(Cc4ccccc4F)CC3)c(-c3cnn(CF)c3)nc2cn1] | 1-{3-[1-(fluoromethyl)-4- pyrazolyl]-2-{4-[(o- fluorophenyl)methyl]-1- piperazinyl}-1,4,6-triaza-7- naphthyl}-2-methyl-1,2- propanediol | NA |
| 607 | 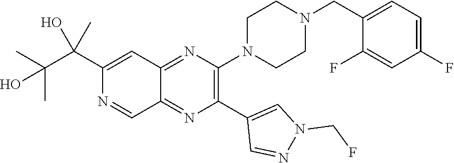 [SMILES: CC(C)(O)C(C)(O)c1cc2nc(N3CCN(Cc4ccc(F)cc4F)CC3)c(-c3cnn(CF)c3)nc2cn1] | 2-(2-{4-[(2,4- difluorophenyl)methyl]-1- piperazinyl}-3-[1-(fluoromethyl)- 4-pyrazolyl]-1,4,6-triaza-7- naphthyl)-3-methyl-2,3-butanediol | NA |
| 608 | 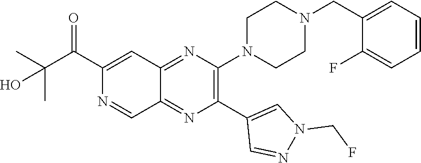 [SMILES: CC(C)(O)C(=O)c1cc2nc(N3CCN(Cc4ccccc4F)CC3)c(-c3cnn(CF)c3)nc2cn1] | 1-{3-[1-(fluoromethyl)-4- pyrazolyl]-2-{4-[(o- fluorophenyl)methyl]-1- piperazinyl}-1,4,6-triaza-7- naphthyl}-2-hydroxy-2-methyl-1- propanone | NA |
| 609 | 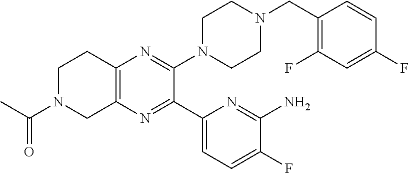 [SMILES: CC(=O)N1CCc2nc(N3CCN(Cc4ccc(F)cc4F)CC3)c(-c3ccc(F)c(N)n3)nc2C1] | 1-[3-(6-amino-5-fluoro-2-pyridyl)- 2-{4-[(2,4-difluorophenyl)methyl]- 1-piperazinyl}-5,6,7,8-tetrahydro- 1,4,6-triaza-6-naphthyl]-1- ethanone | NA |
| 610 | 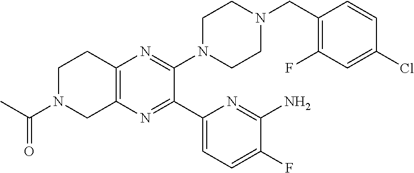 [SMILES: CC(=O)N1CCc2nc(N3CCN(Cc4ccc(Cl)cc4F)CC3)c(-c3ccc(F)c(N)n3)nc2C1] | 1-[3-(6-amino-5-fluoro-2-pyridyl)- 2-{4-[(4-chloro-2- fluorophenyl)methyl]-1- piperazinyl}-5,6,7,8-tetrahydro- 1,4,6-triaza-6-naphthyl]-1- ethanone | NA |
| 611 | 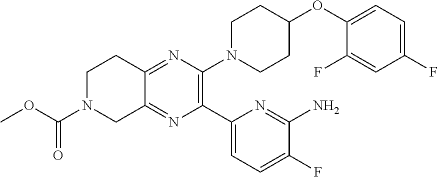 [SMILES: COC(=O)N1CCc2nc(N3CCC(Oc4ccc(F)cc4F)CC3)c(-c3ccc(F)c(N)n3)nc2C1] | 6-{2-[4-(2,4-difluorophenoxy)-1- piperidyl]-6-methoxycarbonyl- 5,6,7,8-tetrahydro-1,4,6-triaza-3- naphthyl}-3-fluoro-2-pyridylamine | NA |
| 612 | 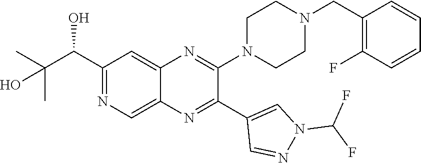 [SMILES: CC(C)(O)[C@H](O)c1cc2nc(N3CCN(Cc4ccccc4F)CC3)c(-c3cnn(C(F)F)c3)nc2cn1] or enantiomer | (R)-1-{3-[1-(difluoromethyl)-4- pyrazolyl]-2-{4-[(o- fluorophenyl)methyl]-1- piperazinyl}-1,4,6-triaza-7- naphthyl}-2-methyl-1,2- propanediol, or enantiomer | (+) Isomer |
| 613 | 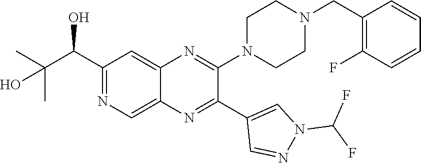 [SMILES: CC(C)(O)[C@@H](O)c1cc2nc(N3CCN(Cc4ccccc4F)CC3)c(-c3cnn(C(F)F)c3)nc2cn1] or enantiomer | (S)-1-{3-[1-(difluoromethyl)-4- pyrazolyl]-2-{4-[(o- fluorophenyl)methyl]-1- piperazinyl}-1,4,6-triaza-7- naphthyl}-2-methyl-1,2- propanediol, or enantiomer | (−) Isomer |
| 614 | 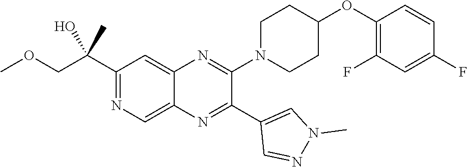 [SMILES: COC[C@](C)(O)c1cc2nc(N3CCC(Oc4ccc(F)cc4F)CC3)c(-c3cnn(C)c3)nc2cn1] or enantiomer | (R)-2-{2-[4-(2,4-difluorophenoxy)- 1-piperidyl]-3-(1-methyl-4- pyrazolyl)-1,4,6-triaza-7- naphthyl}-1-methoxy-2-propanol, or enantiomer | (−) Isomer |
| 615 | 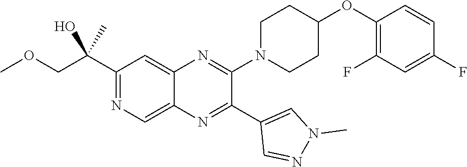 [SMILES: COC[C@@](C)(O)c1cc2nc(N3CCC(Oc4ccc(F)cc4F)CC3)c(-c3cnn(C)c3)nc2cn1] or enantiomer | (S)-2-{2-[4-(2,4-difluorophenoxy)- 1-piperidyl]-3-(1-methyl-4- pyrazolyl)-1,4,6-triaza-7- naphthyl}-1-methoxy-2-propanol, or enantiomer | (+) Isomer |
| 616 | 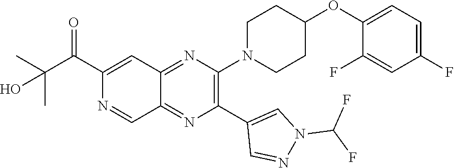 [SMILES: CC(C)(O)C(=O)c1cc2nc(N3CCC(Oc4ccc(F)cc4F)CC3)c(-c3cnn(C(F)F)c3)nc2cn1] | 1-{3-[1-(difluoromethyl)-4- pyrazolyl]-2-[4-(2,4- difluorophenoxy)-1-piperidyl]- 1,4,6-triaza-7-naphthyl}-2- hydroxy-2-methyl-1-propanone | NA |
| 617 | 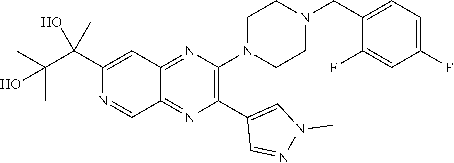 [SMILES: Cn1cc(-c2nc3cnc(C(C)(O)C(C)(C)O)cc3nc2N2CCN(Cc3ccc(F)cc3F)CC2)cn1] | 2-(2-{4-[(2,4- difluorophenyl)methyl]-1- piperazinyl}-3-(1-methyl-4- pyrazolyl)-1,4,6-triaza-7- naphthyl)-3-methyl-2,3-butanediol | NA |
| 618 | 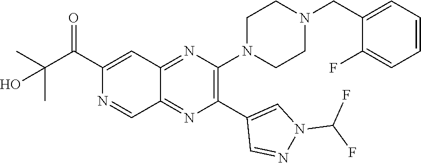 [SMILES: CC(C)(O)C(=O)c1cc2nc(N3CCN(Cc4ccccc4F)CC3)c(-c3cnn(C(F)F)c3)nc2cn1] | 1-{3-[1-(difluoromethyl)-4- pyrazolyl]-2-{4-[(o- fluorophenyl)methyl]-1- piperazinyl}-1,4,6-triaza-7- naphthyl}-2-hydroxy-2-methyl-1- propanone | NA |
| 619 | 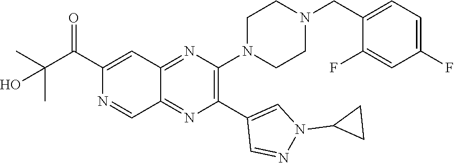 [SMILES: CC(C)(O)C(=O)c1cc2nc(N3CCN(Cc4ccc(F)cc4F)CC3)c(-c3cnn(C4CC4)c3)nc2cn1] | 1-[3-(1-cyclopropyl-4-pyrazolyl)- 2-{4-[(2,4-difluorophenyl)methyl]- 1-piperazinyl}-1,4,6-triaza-7- naphthyl]-2-hydroxy-2-methyl-1- propanone | NA |
| 620 | 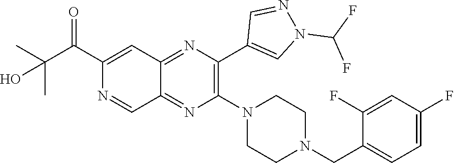 | 1-{2-[1-(difluoromethyl)-4- pyrazolyl]-3-{4-[(2,4- difluorophenyl)methyl]-1- piperazinyl}-1,4,6-triaza-7- naphthyl}-2-hydroxy-2-methyl-1- propanone | NA |
| 621 | 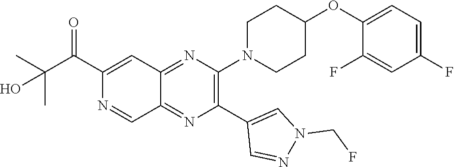 [SMILES: CC(C)(O)C(=O)c1cc2nc(N3CCC(Oc4ccc(F)cc4F)CC3)c(-c3cnn(CF)c3)nc2cn1] | 1-{2-[4-(2,4-difluorophenoxy)-1- piperidyl]-3-[1-(fluoromethyl)-4- pyrazolyl]-1,4,6-triaza-7- naphthyl}-2-hydroxy-2-methyl-1- propanone | NA |
| 622 | 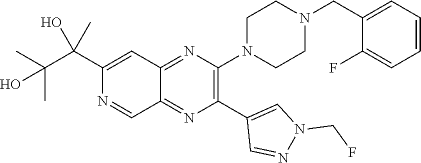 [SMILES: CC(C)(O)C(C)(O)c1cc2nc(N3CCN(Cc4ccccc4F)CC3)c(-c3cnn(CF)c3)nc2cn1] | 2-{3-[1-(fluoromethyl)-4- pyrazolyl]-2-{4-[(o- fluorophenyl)methyl]-1- piperazinyl}-1,4,6-triaza-7- naphthyl}-3-methyl-2,3-butanediol | NA |
| 623 | 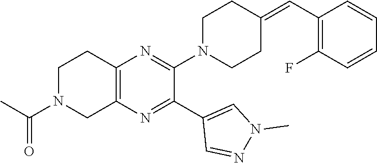 [SMILES: CC(=O)N1CCc2nc(N3CCC(=Cc4ccccc4F)CC3)c(-c3cnn(C)c3)nc2C1] | 1-(2-{4-[(o- fluorophenyl)methylene]-1- piperidyl}-3-(1-methyl-4- pyrazolyl)-5,6,7,8-tetrahydro- 1,4,6-triaza-6-naphthyl)-1- ethanone | NA |
| 624 | 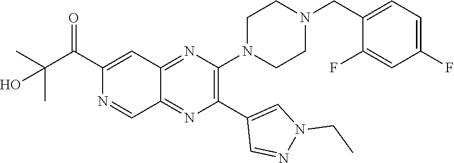 [SMILES: CCn1cc(-c2nc3cnc(C(=O)C(C)(C)O)cc3nc2N2CCN(Cc3ccc(F)cc3F)CC2)cn1] | 1-(2-{4-[(2,4- difluorophenyl)methyl]-1- piperazinyl}-3-(1-ethyl-4- pyrazolyl)-1,4,6-triaza-7- naphthyl)-2-hydroxy-2-methyl-1- propanone | NA |
| 625 | 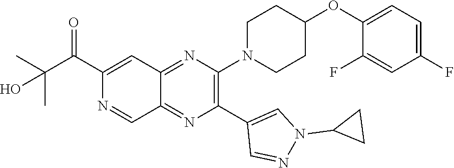 [SMILES: CC(C)(O)C(=O)c1cc2nc(N3CCC(Oc4ccc(F)cc4F)CC3)c(-c3cnn(C4CC4)c3)nc2cn1] | 1-{3-(1-cyclopropyl-4-pyrazolyl)- 2-[4-(2,4-difluorophenoxy)-1- piperidyl]-1,4,6-triaza-7- naphthyl}-2-hydroxy-2-methyl-1- propanone | NA |
| 626 | 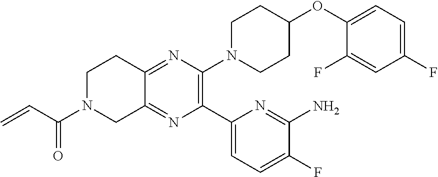 [SMILES: C=CC(=O)N1CCc2nc(N3CCC(Oc4ccc(F)cc4F)CC3)c(-c3ccc(F)c(N)n3)nc2C1] | 1-{3-(6-amino-5-fluoro-2-pyridyl)- 2-[4-(2,4-difluorophenoxy)-1- piperidyl]-5,6,7,8-tetrahydro-1,4,6- triaza-6-naphthyl}-2-propen-1-one | NA |
| 627 | 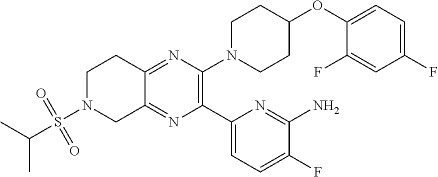 [SMILES: CC(C)S(=O)(=O)N1CCc2nc(N3CCC(Oc4ccc(F)cc4F)CC3)c(-c3ccc(F)c(N)n3)nc2C1] | 6-{2-[4-(2,4-difluorophenoxy)-1- piperidyl]-6-(isopropylsulfonyl)- 5,6,7,8-tetrahydro-1,4,6-triaza-3- naphthyl}-3-fluoro-2-pyridylamine | NA |
| 628 | 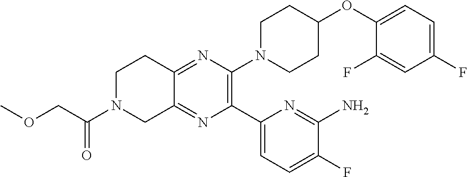 [SMILES: COCC(=O)N1CCc2nc(N3CCC(Oc4ccc(F)cc4F)CC3)c(-c3ccc(F)c(N)n3)nc2C1] | 1-{3-(6-amino-5-fluoro-2-pyridyl)- 2-[4-(2,4-difluorophenoxy)-1- piperidyl]-5,6,7,8-tetrahydro-1,4,6- triaza-6-naphthyl}-2-methoxy-1- ethanone | NA |
| 629 | 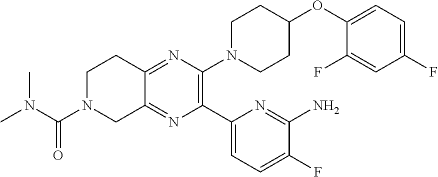 [SMILES: CN(C)C(=O)N1CCc2nc(N3CCC(Oc4ccc(F)cc4F)CC3)c(-c3ccc(F)c(N)n3)nc2C1] | 6-{6-(N,N-dimethylcarbamoyl)-2- [4-(2,4-difluorophenoxy)-1- piperidyl]-5,6,7,8-tetrahydro-1,4,6- triaza-3-naphthyl}-3-fluoro-2- pyridylamine | NA |
| 630 | 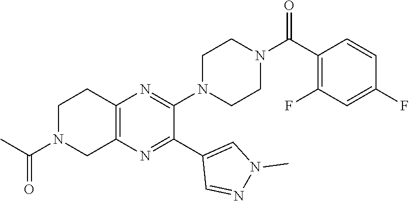 [SMILES: CC(=O)N1CCc2nc(N3CCN(C(=O)c4ccc(F)cc4F)CC3)c(-c3cnn(C)c3)nc2C1] | 1-{2-[4-(2,4-difluorobenzoyl)-1- piperazinyl]-3-(1-methyl-4- pyrazolyl)-5,6,7,8-tetrahydro- 1,4,6-triaza-6-naphthyl}-1- ethanone | NA |
| 631 | 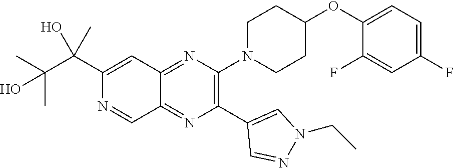 [SMILES: CCn1cc(-c2nc3cnc(C(C)(O)C(C)(C)O)cc3nc2N2CCC(Oc3ccc(F)cc3F)CC2)cn1] | 2-{2-[4-(2,4-difluorophenoxy)-1- piperidyl]-3-(1-ethyl-4-pyrazolyl)- 1,4,6-triaza-7-naphthyl}-3-methyl- 2,3-butanediol | NA |
| 632 | 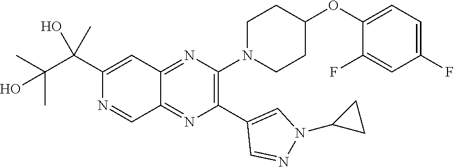 [SMILES: CC(C)(O)C(C)(O)c1cc2nc(N3CCC(Oc4ccc(F)cc4F)CC3)c(-c3cnn(C4CC4)c3)nc2cn1] | 2-{3-(1-cyclopropyl-4-pyrazolyl)- 2-[4-(2,4-difluorophenoxy)-1- piperidyl]-1,4,6-triaza-7- naphthyl}-3-methyl-2,3-butanediol | NA |
| 633 | 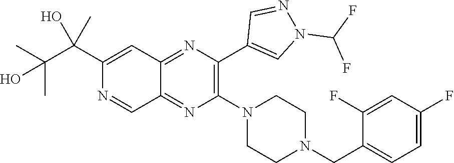 | 2-{2-[1-(difluoromethyl)-4- pyrazolyl]-3-{4-[(2,4- difluorophenyl)methyl]-1- piperazinyl}-1,4,6-triaza-7- naphthyl}-3-methyl-2,3-butanediol | NA |
| 634 | 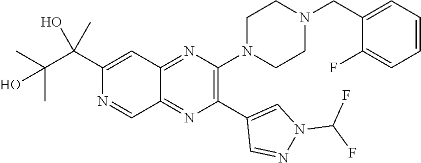 [SMILES: CC(C)(O)C(C)(O)c1cc2nc(N3CCN(Cc4ccccc4F)CC3)c(-c3cnn(C(F)F)c3)nc2cn1] | 2-{3-[1-(difluoromethyl)-4- pyrazolyl]-2-{4-[(o- fluorophenyl)methyl]-1- piperazinyl}-1,4,6-triaza-7- naphthyl}-3-methyl-2,3-butanediol | NA |
| 635 | 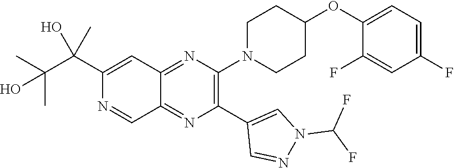 [SMILES: CC(C)(O)C(C)(O)c1cc2nc(N3CCC(Oc4ccc(F)cc4F)CC3)c(-c3cnn(C(F)F)c3)nc2cn1] | 2-{3-[1-(difluoromethyl)-4- pyrazolyl]-2-[4-(2,4- difluorophenoxy)-1-piperidyl]- 1,4,6-triaza-7-naphthyl}-3-methyl- 2,3-butanediol | NA |
| 636 | 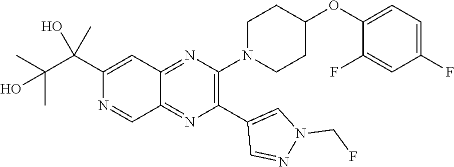 [SMILES: CC(C)(O)C(C)(O)c1cc2nc(N3CCC(Oc4ccc(F)cc4F)CC3)c(-c3cnn(CF)c3)nc2cn1] | 2-{2-[4-(2,4-difluorophenoxy)-1- piperidyl]-3-[1-(fluoromethyl)-4- pyrazolyl]-1,4,6-triaza-7- naphthyl}-3-methyl-2,3-butanediol | NA |
| 637 | 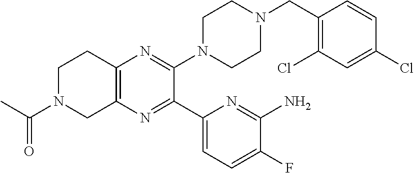 [SMILES: CC(=O)N1CCc2nc(N3CCN(Cc4ccc(Cl)cc4Cl)CC3)c(-c3ccc(F)c(N)n3)nc2C1] | 1-[3-(6-amino-5-fluoro-2-pyridyl)- 2-{4-[(2,4-dichlorophenyl)methyl]- 1-piperazinyl}-5,6,7,8-tetrahydro- 1,4,6-triaza-6-naphthyl]-1- ethanone | NA |
| 638 | 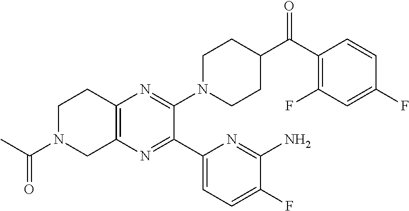 [SMILES: CC(=O)N1CCc2nc(N3CCC(C(=O)c4ccc(F)cc4F)CC3)c(-c3ccc(F)c(N)n3)nc2C1] | 1-{3-(6-amino-5-fluoro-2-pyridyl)- 2-[4-(2,4-difluorobenzoyl)-1- piperidyl]-5,6,7,8-tetrahydro-1,4,6- triaza-6-naphthyl}-1-ethanone | NA |
| 639 | 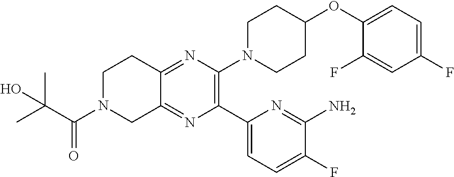 [SMILES: CC(C)(O)C(=O)N1CCc2nc(N3CCC(Oc4ccc(F)cc4F)CC3)c(-c3ccc(F)c(N)n3)nc2C1] | 1-{3-(6-amino-5-fluoro-2-pyridyl)- 2-[4-(2,4-difluorophenoxy)-1- piperidyl]-5,6,7,8-tetrahydro-1,4,6- triaza-6-naphthyl}-2-hydroxy-2- methyl-1-propanone | NA |
| 640 | 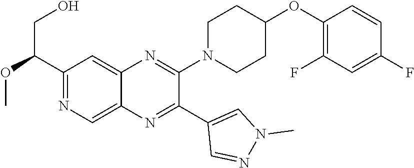 [SMILES: CO[C@@H](CO)c1cc2nc(N3CCC(Oc4ccc(F)cc4F)CC3)c(-c3cnn(C)c3)nc2cn1] or enantiomer | (R)-2-{2-[4-(2,4-difluorophenoxy)- 1-piperidyl]-3-(1-methyl-4- pyrazolyl)-1,4,6-triaza-7- naphthyl}-2-methoxyethanol, or enantiomer | (+) Isomer |
| 641 | 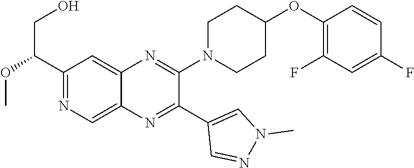 [SMILES: CO[C@H](CO)c1cc2nc(N3CCC(Oc4ccc(F)cc4F)CC3)c(-c3cnn(C)c3)nc2cn1] or enantiomer | (S)-2-{2-[4-(2,4-difluorophenoxy)- 1-piperidyl]-3-(1-methyl-4- pyrazolyl)-1,4,6-triaza-7- naphthyl}-2-methoxyethanol, or enantiomer | (−) Isomer |
| 642 | 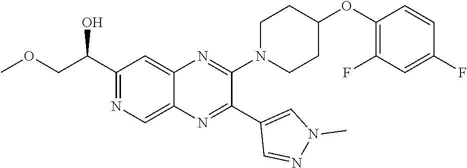 [SMILES: COC[C@@H](O)c1cc2nc(N3CCC(Oc4ccc(F)cc4F)CC3)c(-c3cnn(C)c3)nc2cn1] or enantiomer | (S)-1-{2-[4-(2,4-difluorophenoxy)- 1-piperidyl]-3-(1-methyl-4- pyrazolyl)-1,4,6-triaza-7- naphthyl}-2-methoxy-1-ethanol, or enantiomer | (−) Isomer |
| 643 | 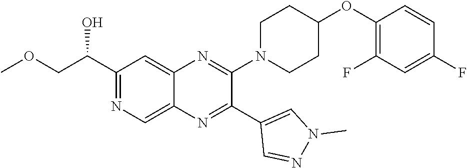 [SMILES: COC[C@H](O)c1cc2nc(N3CCC(Oc4ccc(F)cc4F)CC3)c(-c3cnn(C)c3)nc2cn1] or enantiomer | (R)-1-{2-[4-(2,4-difluorophenoxy)- 1-piperidyl]-3-(1-methyl-4- pyrazolyl)-1,4,6-triaza-7- naphthyl}-2-methoxy-1-ethanol, or enantiomer | (+) Isomer |
| 644 | 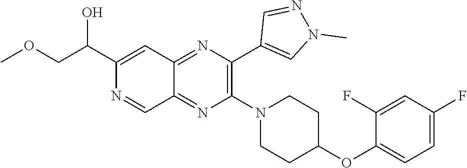 [SMILES: COCC(O)c1cc2nc(-c3cnn(C)c3)c(N3CCC(Oc4ccc(F)cc4F)CC3)nc2cn1] | 1-{3-[4-(2,4-difluorophenoxy)-1- piperidyl]-2-(1-methyl-4- pyrazolyl)-1,4,6-triaza-7- naphthyl}-2-methoxy-1-ethanol | NA |
| 645 | 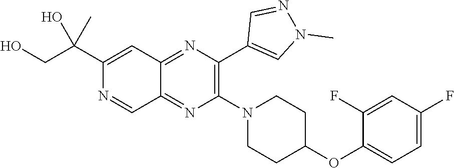 | 2-{3-[4-(2,4-difluorophenoxy)-1- piperidyl]-2-(1-methyl-4- pyrazolyl)-1,4,6-triaza-7- naphthyl}-1,2-propanediol | NA |
| 646 | 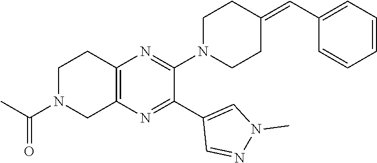 [SMILES: CC(=O)N1CCc2nc(N3CCC(=Cc4ccccc4)CC3)c(-c3cnn(C)c3)nc2C1] | 1-[2-(4-benzylidene-1-piperidyl)-3- (1-methyl-4-pyrazolyl)-5,6,7,8- tetrahydro-1,4,6-triaza-6- naphthyl]-1-ethanone | NA |
| 647 | 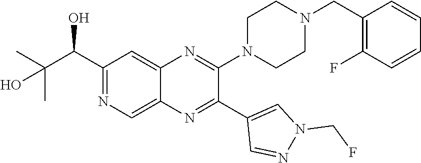 [SMILES: CC(C)(O)[C@@H](O)c1cc2nc(N3CCN(Cc4ccccc4F)CC3)c(-c3cnn(CF)c3)nc2cn1] or enantiomer | (S)-1-{3-[1-(fluoromethyl)-4- pyrazolyl]-2-{4-[(o- fluorophenyl)methyl]-1- piperazinyl}-1,4,6-triaza-7- naphthyl}-2-methyl-1,2- propanediol, or enantiomer | (−) Isomer |
| 648 | 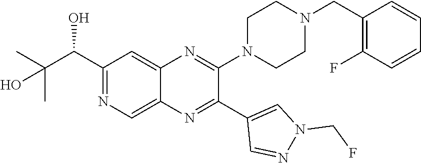 [SMILES: CC(C)(O)[C@H](O)c1cc2nc(N3CCN(Cc4ccccc4F)CC3)c(-c3cnn(CF)c3)nc2cn1] or enantiomer | (R)-1-{3-[1-(fluoromethyl)-4- pyrazolyl]-2-{4-[(o- fluorophenyl)methyl]-1- piperazinyl}-1,4,6-triaza-7- naphthyl}-2-methyl-1,2- propanediol, or enantiomer | (+) Isomer |
| 649 | 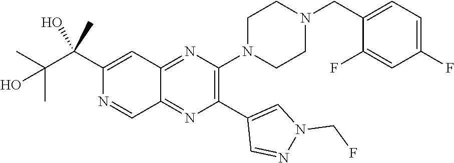 [SMILES: CC(C)(O)[C@](C)(O)c1cc2nc(N3CCN(Cc4ccc(F)cc4F)CC3)c(-c3cnn(CF)c3)nc2cn1] or enantiomer | (R)-2-(2-{4-[(2,4- difluorophenyl)methyl]-1- piperazinyl}-3-[1-(fluoromethyl)- 4-pyrazolyl]-1,4,6-triaza-7- naphthyl)-3-methyl-2,3-butanediol, or enantiomer | (+) Isomer |
| 650 | 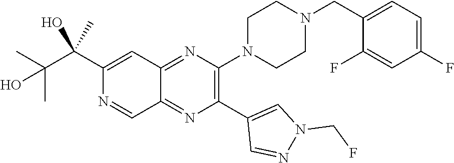 [SMILES: CC(C)(O)[C@@](C)(O)c1cc2nc(N3CCN(Cc4ccc(F)cc4F)CC3)c(-c3cnn(CF)c3)nc2cn1] or enantiomer | (S)-2-(2-{4-[(2,4- difluorophenyl)methyl]-1- piperazinyl}-3-[1-(fluoromethyl)- 4-pyrazolyl]-1,4,6-triaza-7- naphthyl)-3-methyl-2,3-butanediol, or enantiomer | (−) Isomer |
| 651 | 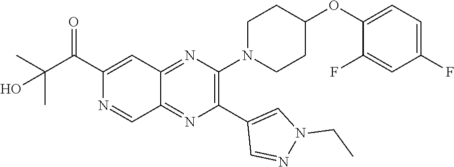 [SMILES: CCn1cc(-c2nc3cnc(C(=O)C(C)(C)O)cc3nc2N2CCC(Oc3ccc(F)cc3F)CC2)cn1] | 1-{2-[4-(2,4-difluorophenoxy)-1- piperidyl]-3-(1-ethyl-4-pyrazolyl)- 1,4,6-triaza-7-naphthyl}-2- hydroxy-2-methyl-1-propanone | NA |
| 652 | 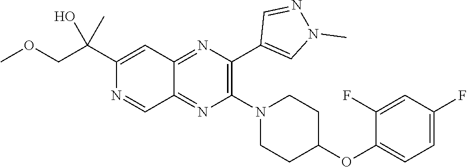 [SMILES: COCC(C)(O)c1cc2nc(-c3cnn(C)c3)c(N3CCC(Oc4ccc(F)cc4F)CC3)nc2cn1] | 2-{3-[4-(2,4-difluorophenoxy)-1- piperidyl]-2-(1-methyl-4- pyrazolyl)-1,4,6-triaza-7- naphthyl}-1-methoxy-2-propanol | NA |
| 653 | 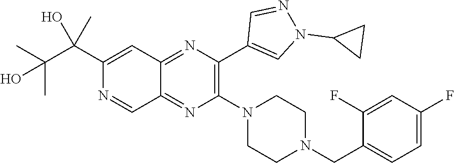 [SMILES: CC(C)(O)C(C)(O)c1cc2nc(-c3cnn(C4CC4)c3)c(N3CCN(Cc4ccc(F)cc4F)CC3)nc2cn1] | 2-[2-(1-cyclopropyl-4-pyrazolyl)- 3-{4-[(2,4-difluorophenyl)methyl]- 1-piperazinyl}-1,4,6-triaza-7- naphthyl]-3-methyl-2,3-butanediol | NA |
| 654 | 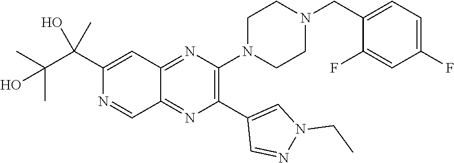 [SMILES: CCn1cc(-c2nc3cnc(C(C)(O)C(C)(C)O)cc3nc2N2CCN(Cc3ccc(F)cc3F)CC2)cn1] | 2-(2-{4-[(2,4- difluorophenyl)methyl]-1- piperazinyl}-3-(1-ethyl-4- pyrazolyl)-1,4,6-triaza-7- naphthyl)-3-methyl-2,3-butanediol | NA |
| 655 | 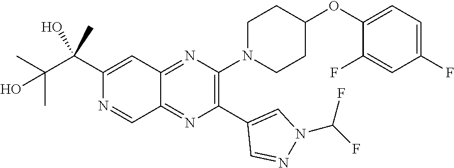 [SMILES: CC(C)(O)[C@](C)(O)c1cc2nc(N3CCC(Oc4ccc(F)cc4F)CC3)c(-c3cnn(C(F)F)c3)nc2cn1] or enantiomer | (R)-2-{3-[1-(difluoromethyl)-4- pyrazolyl]-2-[4-(2,4- difluorophenoxy)-1-piperidyl]- 1,4,6-triaza-7-naphthyl}-3-methyl- 2,3-butanediol, or enantiomer | (+) Isomer |
| 656 | 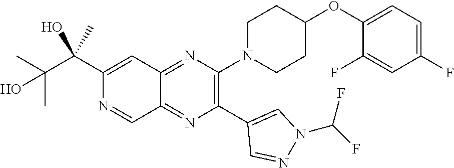 [SMILES: CC(C)(O)[C@@](C)(O)c1cc2nc(N3CCC(Oc4ccc(F)cc4F)CC3)c(-c3cnn(C(F)F)c3)nc2cn1] or enantiomer | (S)-2-{3-[1-(difluoromethyl)-4- pyrazolyl]-2-[4-(2,4- difluorophenoxy)-1-piperidyl]- 1,4,6-triaza-7-naphthyl}-3-methyl- 2,3-butanediol, or enantiomer | (−) Isomer |
| 657 | 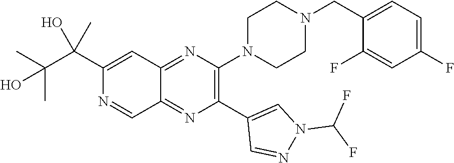 [SMILES: CC(C)(O)C(C)(O)c1cc2nc(N3CCN(Cc4ccc(F)cc4F)CC3)c(-c3cnn(C(F)F)c3)nc2cn1] | 2-{3-[1-(difluoromethyl)-4- pyrazolyl]-2-{4-[(2,4- difluorophenyl)methyl]-1- piperazinyl}-1,4,6-triaza-7- naphthyl}-3-methyl-2,3-butanediol | NA |
| 658 | 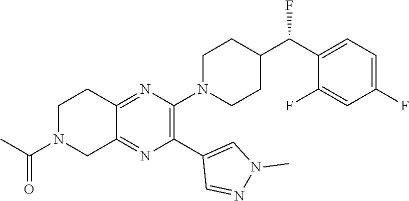 [SMILES: CC(=O)N1CCc2nc(N3CCC([C@H](F)c4ccc(F)cc4F)CC3)c(-c3cnn(C)c3)nc2C1] or enantiomer | 1-(2-{4-[(S)-(2,4- difluorophenyl)fluoromethyl]-1- piperidyl}-3-(1-methyl-4- pyrazolyl)-5,6,7,8-tetrahydro- 1,4,6-triaza-6-naphthyl)-1- ethanone, or enantiomer | (+) Isomer |
| 659 | 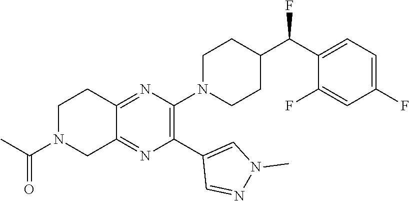 [SMILES: CC(=O)N1CCc2nc(N3CCC([C@@H](F)c4ccc(F)cc4F)CC3)c(-c3cnn(C)c3)nc2C1] or enantiomer | 1-(2-{4-[(R)-(2,4- difluorophenyl)fluoromethyl]-1- piperidyl}-3-(1-methyl-4- pyrazolyl)-5,6,7,8-tetrahydro- 1,4,6-triaza-6-naphthyl)-1- ethanone, or enantiomer | (−) Isomer |
| 660 | 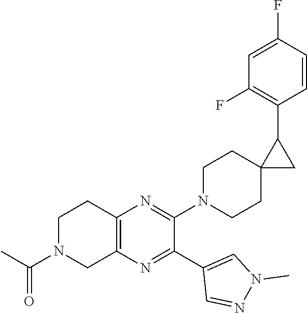 [SMILES: CC(=O)N1CCc2nc(N3CCC4(CC3)CC4c3ccc(F)cc3F)c(-c3cnn(C)c3)nc2C1] | 1-(2-{1-(2,4-difluorophenyl)-6- aza-6-spiro[2.5]octyl}-3-(1- methyl-4-pyrazolyl)-5,6,7,8- tetrahydro-1,4,6-triaza-6- naphthyl)-1-ethanone | NA |
| 661 | 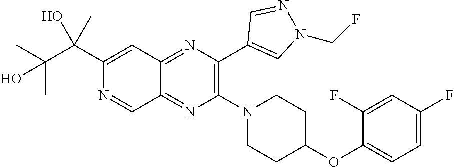 | 2-{3-[4-(2,4-difluorophenoxy)-1- piperidyl]-2-[1-(fluoromethyl)-4- pyrazolyl]-1,4,6-triaza-7- naphthyl}-3-methyl-2,3-butanediol | NA |
| 662 | 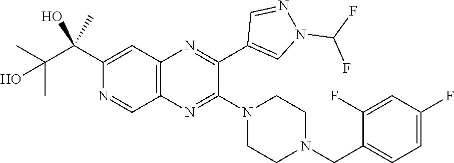 or enantiomer | (S)-2-{2-[1-(difluoromethyl)-4- pyrazolyl]-3-{4-[(2,4- difluorophenyl)methyl]-1- piperazinyl}-1,4,6-triaza-7- naphthyl}-3-methyl-2,3- butanediol, or enantiomer | (+) Isomer |
| 663 | 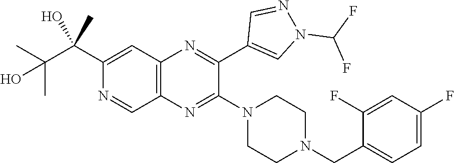 or enantiomer | (R)-2-{2-[1-(difluoromethyl)-4- pyrazolyl]-3-{4-[(2,4- difluorophenyl)methyl]-1- piperazinyl}-1,4,6-triaza-7- naphthyl}-3-methyl-2,3- butanediol, or enantiomer | (−) Isomer |
| 664 | 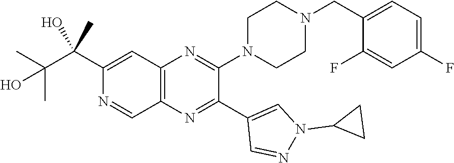 [SMILES: CC(C)(O)[C@](C)(O)c1cc2nc(N3CCN(Cc4ccc(F)cc4F)CC3)c(-c3cnn(C4CC4)c3)nc2cn1] or enantiomer | (R)-2-{3-(1-cyclopropyl-4- pyrazolyl)-2-[4-(2,4- difluorophenoxy)-1-piperidyl]- 1,4,6-triaza-7-naphthyl}-3-methyl- 2,3-butanediol, or enantiomer | (−) Isomer |
| 665 | 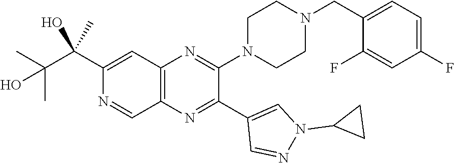 [SMILES: CC(C)(O)[C@@](C)(O)c1cc2nc(N3CCN(Cc4ccc(F)cc4F)CC3)c(-c3cnn(C4CC4)c3)nc2cn1] or enantiomer | (S)-2-{3-(1-cyclopropyl-4- pyrazolyl)-2-[4-(2,4- difluorophenoxy)-1-piperidyl]- 1,4,6-triaza-7-naphthyl}-3-methyl- 2,3-butanediol, or enantiomer | (+) Isomer |
| 666 | 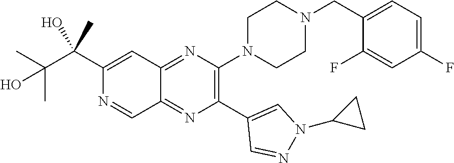 [SMILES: CC(C)(O)[C@](C)(O)c1cc2nc(N3CCN(Cc4ccc(F)cc4F)CC3)c(-c3cnn(C4CC4)c3)nc2cn1] or enantiomer | (R)-2-[3-(1-cyclopropyl-4- pyrazolyl)-2-{4-[(2,4- difluorophenyl)methyl]-1- piperazinyl}-1,4,6-triaza-7- naphthyl]-3-methyl-2,3-butanediol, or enantiomer | (−) Isomer |
| 667 | 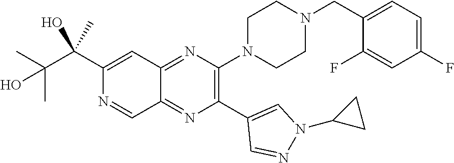 [SMILES: CC(C)(O)[C@@](C)(O)c1cc2nc(N3CCN(Cc4ccc(F)cc4F)CC3)c(-c3cnn(C4CC4)c3)nc2cn1] or enantiomer | (S)-2-[3-(1-cyclopropyl-4- pyrazolyl)-2-{4-[(2,4- difluorophenyl)methyl]-1- piperazinyl}-1,4,6-triaza-7- naphthyl]-3-methyl-2,3-butanediol, or enantiomer | (+) Isomer |
| 668 | 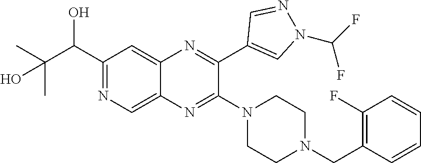 [SMILES: CC(C)(O)C(O)c1cc2nc(-c3cnn(C(F)F)c3)c(N3CCN(Cc4ccccc4F)CC3)nc2cn1] or enantiomer | 1-{2-[1-(difluoromethyl)-4- pyrazolyl]-3-{4-[(o- fluorophenyl)methyl]-1- piperazinyl}-1,4,6-triaza-7- naphthyl}-2-methyl-1,2- propanediol | NA |
| 669 | 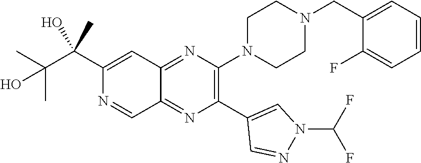 [SMILES: CC(C)(O)[C@](C)(O)c1cc2nc(N3CCN(Cc4ccccc4F)CC3)c(-c3cnn(C(F)F)c3)nc2cn1] or enantiomer | (R)-2-{3-[1-(difluoromethyl)-4- pyrazolyl]-2-{4-[(o- fluorophenyl)methyl]-1- piperazinyl}-1,4,6-triaza-7- naphthyl}-3-methyl-2,3- butanediol, or enantiomer | (+) Isomer |
| 670 | 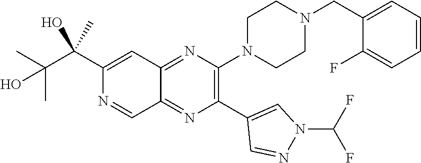 [SMILES: CC(C)(O)[C@@](C)(O)c1cc2nc(N3CCN(Cc4ccccc4F)CC3)c(-c3cnn(C(F)F)c3)nc2cn1] or enantiomer | (S)-2-{3-[1-(difluoromethyl)-4- pyrazolyl]-2-{4-[(o- fluorophenyl)methyl]-1- piperazinyl}-1,4,6-triaza-7- naphthyl}-3-methyl-2,3- butanediol, or enantiomer | (−) Isomer |
| 671 | 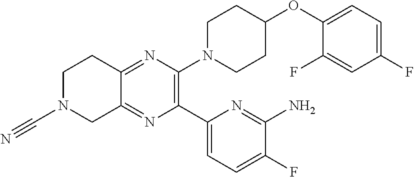 [SMILES: N#CN1CCc2nc(N3CCC(Oc4ccc(F)cc4F)CC3)c(-c3ccc(F)c(N)n3)nc2C1] | 6-{6-cyano-2-[4-(2,4- difluorophenoxy)-1-piperidyl]- 5,6,7,8-tetrahydro-1,4,6-triaza-3- naphthyl}-3-fluoro-2-pyridylamine | NA |
| 672 | 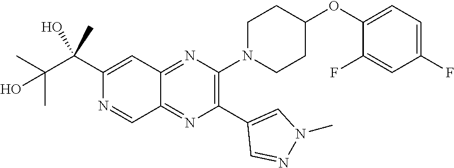 [SMILES: Cn1cc(-c2nc3cnc([C@@](C)(O)C(C)(C)O)cc3nc2N2CCC(Oc3ccc(F)cc3F)CC2)cn1] or enantiomer | (R)-2-{2-[4-(2,4-difluorophenoxy)- 1-piperidyl]-3-(1-methyl-4- pyrazolyl)-1,4,6-triaza-7- naphthyl}-3-methyl-2,3- butanediol, or enantiomer | (−) Isomer |
| 673 | 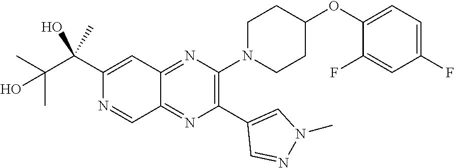 [SMILES: Cn1cc(-c2nc3cnc([C@](C)(O)C(C)(C)O)cc3nc2N2CCC(Oc3ccc(F)cc3F)CC2)cn1] or enantiomer | (S)-2-{2-[4-(2,4-difluorophenoxy)- 1-piperidyl]-3-(1-methyl-4- pyrazolyl)-1,4,6-triaza-7- naphthyl}-3-methyl-2,3- butanediol, or enantiomer | (+) Isomer |
| 674 | 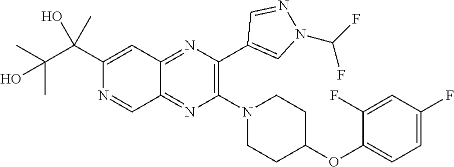 | 2-{2-[1-(difluoromethyl)-4- pyrazolyl]-3-[4-(2,4- difluorophenoxy)-1-piperidyl]- 1,4,6-triaza-7-naphthyl}-3-methyl- 2,3-butanediol | NA |
| 675 | 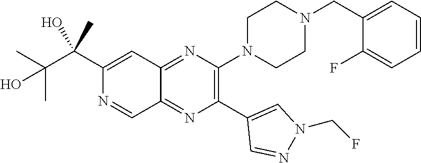 [SMILES: CC(C)(O)[C@](C)(O)c1cc2nc(N3CCN(Cc4ccccc4F)CC3)c(-c3cnn(CF)c3)nc2cn1] or enantiomer | (R)-2-{3-[1-(fluoromethyl)-4- pyrazolyl]-2-{4-[(o- fluorophenyl)methyl]-1- piperazinyl}-1,4,6-triaza-7- naphthyl}-3-methyl-2,3- butanediol, or enantiomer | (+) Isomer |
| 676 | 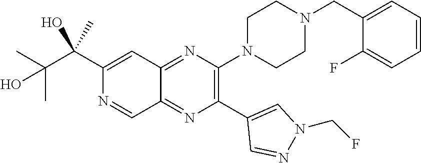 [SMILES: CC(C)(O)[C@@](C)(O)c1cc2nc(N3CCN(Cc4ccccc4F)CC3)c(-c3cnn(CF)c3)nc2cn1] or enantiomer | (S)-2-{3-[1-(fluoromethyl)-4- pyrazolyl]-2-{4-[(o- fluorophenyl)methyl]-1- piperazinyl}-1,4,6-triaza-7- naphthyl}-3-methyl-2,3- butanediol, or enantiomer | (−) Isomer |
| 677 | 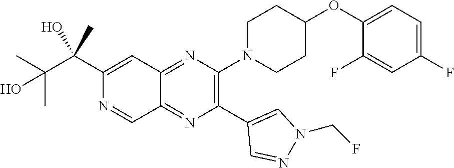 [SMILES: CC(C)(O)[C@](C)(O)c1cc2nc(N3CCC(Oc4ccc(F)cc4F)CC3)c(-c3cnn(CF)c3)nc2cn1] or enantiomer | (R)-2-{2-[4-(2,4-difluorophenoxy)- 1-piperidyl]-3-[1-(fluoromethyl)-4- pyrazolyl]-1,4,6-triaza-7- naphthyl}-3-methyl-2,3- butanediol, or enantiomer | (+) Isomer |
| 678 | 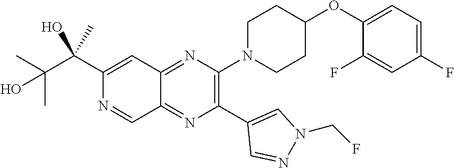 [SMILES: CC(C)(O)[C@@](C)(O)c1cc2nc(N3CCC(Oc4ccc(F)cc4F)CC3)c(-c3cnn(CF)c3)nc2cn1] or enantiomer | (S)-2-{2-[4-(2,4-difluorophenoxy)- 1-piperidyl]-3-[1-(fluoromethyl)-4- pyrazolyl]-1,4,6-triaza-7- naphthyl}-3-methyl-2,3- butanediol, or enantiomer | (−) Isomer |
| 679 | 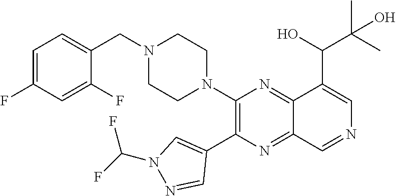 | 1-{3-[1-(difluoromethyl)-4- pyrazolyl]-2-{4-[(2,4- difluorophenyl)methyl]-1- piperazinyl}-1,4,6-triaza-8- naphthyl}-2-methyl-1,2- propanediol | NA |
| 680 | 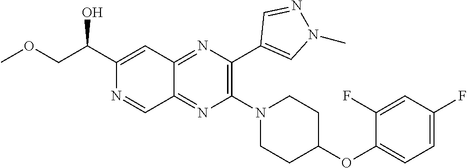 [SMILES: COC[C@@H](O)c1cc2nc(-c3cnn(C)c3)c(N3CCC(Oc4ccc(F)cc4F)CC3)nc2cn1] or enantiomer | (S)-1-{3-[4-(2,4-difluorophenoxy)- 1-piperidyl]-2-(1-methyl-4- pyrazolyl)-1,4,6-triaza-7- naphthyl}-2-methoxy-1-ethanol, or enantiomer | (+) Isomer |
| 681 | 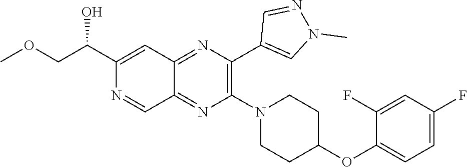 [SMILES: COC[C@H](O)c1cc2nc(-c3cnn(C)c3)c(N3CCC(Oc4ccc(F)cc4F)CC3)nc2cn1] or enantiomer | (R)-1-{3-[4-(2,4-difluorophenoxy)- 1-piperidyl]-2-(1-methyl-4- pyrazolyl)-1,4,6-triaza-7- naphthyl}-2-methoxy-1-ethanol, or enantiomer | (−) Isomer |
| 682 | 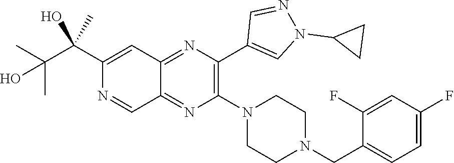 [SMILES: CC(C)(O)[C@@](C)(O)c1cc2nc(-c3cnn(C4CC4)c3)c(N3CCN(Cc4ccc(F)cc4F)CC3)nc2cn1] or enantiomer | (S)-2-[2-(1-cyclopropyl-4- pyrazolyl)-3-{4-[(2,4- difluorophenyl)methyl]-1- piperazinyl}-1,4,6-triaza-7- naphthyl]-3-methyl-2,3-butanediol, or enantiomer | (+) Isomer |
| 683 | 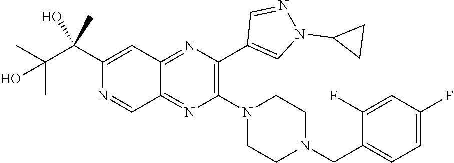 [SMILES: CC(C)(O)[C@](C)(O)c1cc2nc(-c3cnn(C4CC4)c3)c(N3CCN(Cc4ccc(F)cc4F)CC3)nc2cn1] | (R)-2-[2-(1-cyclopropyl-4- pyrazolyl)-3-{4-[(2,4- difluorophenyl)methyl]-1- piperazinyl}-1,4,6-triaza-7- naphthyl]-3-methyl-2,3-butanediol | (−) Isomer |
| 684 | 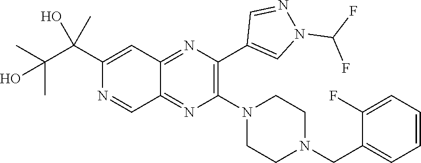 [SMILES: CC(C)(O)C(C)(O)c1cc2nc(-c3cnn(C(F)F)c3)c(N3CCN(Cc4ccccc4F)CC3)nc2cn1] | 2-{2-[1-(difluoromethyl)-4- pyrazolyl]-3-{4-[(o- fluorophenyl)methyl]-1- piperazinyl}-1,4,6-triaza-7- naphthyl}-3-methyl-2,3-butanediol | NA |
| 685 | 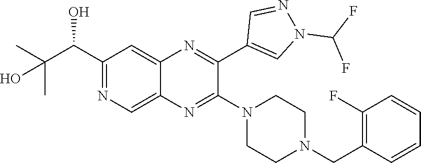 [SMILES: CC(C)(O)[C@H](O)c1cc2nc(-c3cnn(C(F)F)c3)c(N3CCN(Cc4ccccc4F)CC3)nc2cn1] or enantiomer | (R)-1-{2-[1-(difluoromethyl)-4- pyrazolyl]-3-{4-[(o- fluorophenyl)methyl]-1- piperazinyl}-1,4,6-triaza-7- naphthyl}-2-methyl-1,2- propanediol, or enantiomer | (−) Isomer |
| 686 | 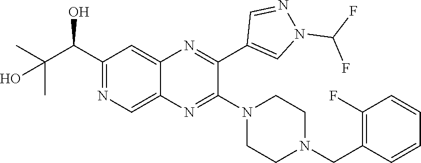 [SMILES: CC(C)(O)[C@@H](O)c1cc2nc(-c3cnn(C(F)F)c3)c(N3CCN(Cc4ccccc4F)CC3)nc2cn1] or enantiomer | (S)-1-{2-[1-(difluoromethyl)-4- pyrazolyl]-3-{4-[(o- fluorophenyl)methyl]-1- piperazinyl}-1,4,6-triaza-7- naphthyl}-2-methyl-1,2- propanediol, or enantiomer | (+) Isomer |
| 687 | 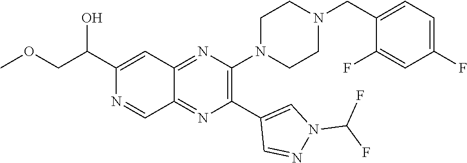 [SMILES: COCC(O)c1cc2nc(N3CCN(Cc4ccc(F)cc4F)CC3)c(-c3cnn(C(F)F)c3)nc2cn1] | 1-{3-[1-(difluoromethyl)-4- pyrazolyl]-2-{4-[(2,4- difluorophenyl)methyl]-1- piperazinyl}-1,4,6-triaza-7- naphthyl}-2-methoxy-1-ethanol | NA |
| 688 | 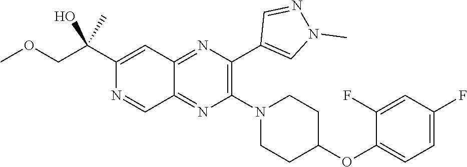 [SMILES: COC[C@@](C)(O)c1cc2nc(-c3cnn(C)c3)c(N3CCC(Oc4ccc(F)cc4F)CC3)nc2cn1] or enantiomer | (S)-2-{3-[4-(2,4-difluorophenoxy)- 1-piperidyl]-2-(1-methyl-4- pyrazolyl)-1,4,6-triaza-7- naphthyl}-1-methoxy-2-propanol, or enantiomer | (−) Isomer |
| 689 | 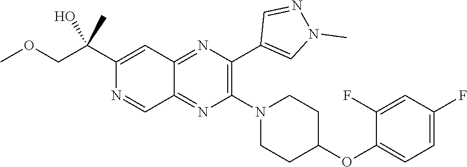 [SMILES: COC[C@](C)(O)c1cc2nc(-c3cnn(C)c3)c(N3CCC(Oc4ccc(F)cc4F)CC3)nc2cn1] or enantiomer | (R)-2-{3-[4-(2,4-difluorophenoxy)- 1-piperidyl]-2-(1-methyl-4- pyrazolyl)-1,4,6-triaza-7- naphthyl}-1-methoxy-2-propanol, or enantiomer | (+) Isomer |
| 690 | 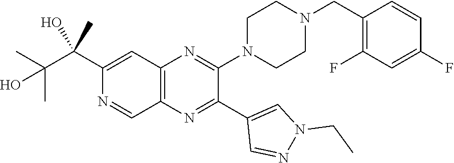 [SMILES: CCn1cc(-c2nc3cnc([C@@](C)(O)C(C)(C)O)cc3nc2N2CCN(Cc3ccc(F)cc3F)CC2)cn1] or enantiomer | (R)-2-(2-{4-[(2,4- difluorophenyl)methyl]-1- piperazinyl}-3-(1-ethyl-4- pyrazolyl)-1,4,6-triaza-7- naphthyl)-3-methyl-2,3-butanediol, or enantiomer | (−) Isomer |
| 691 | 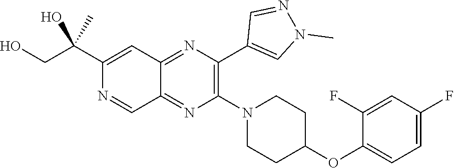 [SMILES: Cn1cc(-c2nc3cc([C@](C)(O)CO)ncc3nc2N2CCC(Oc3ccc(F)cc3F)CC2)cn1] or enantiomer | (S)-2-{3-[4-(2,4-difluorophenoxy)- 1-piperidyl]-2-(1-methyl-4- pyrazolyl)-1,4,6-triaza-7- naphthyl}-1,2-propanediol, or enantiomer | (+) Isomer |
| 692 | 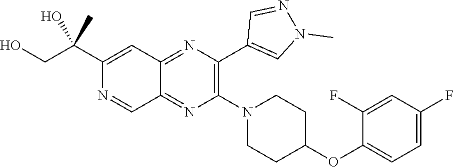 or enantiomer | (R)-2-{3-[4-(2,4-difluorophenoxy)- 1-piperidyl]-2-(1-methyl-4- pyrazolyl)-1,4,6-triaza-7- naphthyl}-1,2-propanediol, or enantiomer | (−) Isomer |
| 693 | 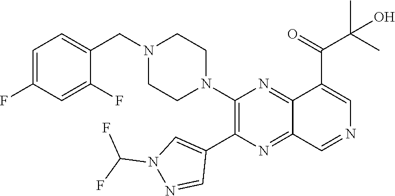 | 1-{3-[1-(difluoromethyl)-4- pyrazolyl]-2-{4-[(2,4- difluorophenyl)methyl]-1- piperazinyl}-1,4,6-triaza-8- naphthyl}-2-hydroxy-2-methyl-1- propanone | NA |
| 694 | 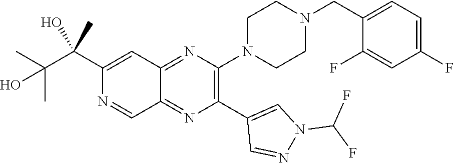 [SMILES: CC(C)(O)[C@](C)(O)c1cc2nc(N3CCN(Cc4ccc(F)cc4F)CC3)c(-c3cnn(C(F)F)c3)nc2cn1] or enantiomer | (R)-2-{3-[1-(difluoromethyl)-4- pyrazolyl]-2-{4-[(2,4- difluorophenyl)methyl]-1- piperazinyl}-1,4,6-triaza-7- naphthyl}-3-methyl-2,3- butanediol, or enantiomer | (+) Isomer |
| 695 | 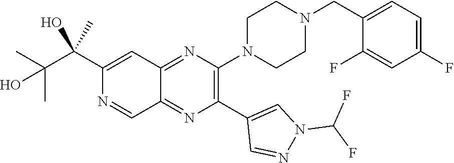 [SMILES: CC(C)(O)[C@@](C)(O)c1cc2nc(N3CCN(Cc4ccc(F)cc4F)CC3)c(-c3cnn(C(F)F)c3)nc2cn1] or enantiomer | (S)-2-{3-[1-(difluoromethyl)-4- pyrazolyl]-2-{4-[(2,4- difluorophenyl)methyl]-1- piperazinyl}-1,4,6-triaza-7- naphthyl}-3-methyl-2,3- butanediol, or enantiomer | (−) Isomer |
| 696 | 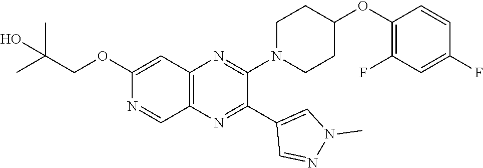 [SMILES: Cn1cc(-c2nc3cnc(OCC(C)(C)O)cc3nc2N2CCC(Oc3ccc(F)cc3F)CC2)cn1] | 1-{2-[4-(2,4-difluorophenoxy)-1- piperidyl]-3-(1-methyl-4- pyrazolyl)-1,4,6-triaza-7- naphthyloxy}-2-methyl-2-propanol | NA |
| 697 | 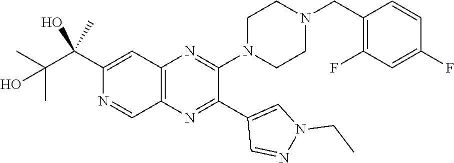 [SMILES: CCn1cc(-c2nc3cnc([C@](C)(O)C(C)(C)O)cc3nc2N2CCN(Cc3ccc(F)cc3F)CC2)cn1] or enantiomer | (S)-2-(2-{4-[(2,4- difluorophenyl)methyl]-1- piperazinyl}-3-(1-ethyl-4- pyrazolyl)-1,4,6-triaza-7- naphthyl)-3-methyl-2,3-butanediol, or enantiomer | (+) Isomer |
| 698 | 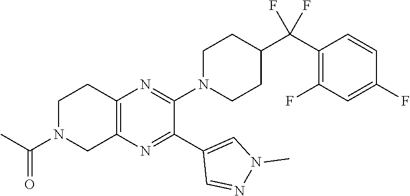 [SMILES: CC(=O)N1CCc2nc(N3CCC(C(F)(F)c4ccc(F)cc4F)CC3)c(-c3cnn(C)c3)nc2C1] | 1-(2-{4-[(2,4- difluorophenyl)difluoromethyl]-1- piperidyl}-3-(1-methyl-4- pyrazolyl)-5,6,7,8-tetrahydro- 1,4,6-triaza-6-naphthyl)-1- ethanone | NA |
| 699 | 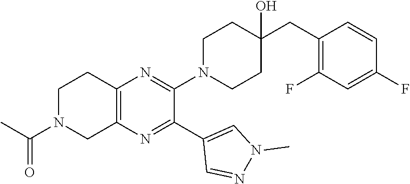 [SMILES: CC(=O)N1CCc2nc(N3CCC(O)(Cc4ccc(F)cc4F)CC3)c(-c3cnn(C)c3)nc2C1] | 1-(2-{4-[(2,4- difluorophenyl)methyl]-4-hydroxy- 1-piperidyl}-3-(1-methyl-4- pyrazolyl)-5,6,7,8-tetrahydro- 1,4,6-triaza-6-naphthyl)-1- ethanone | NA |
| 700 | 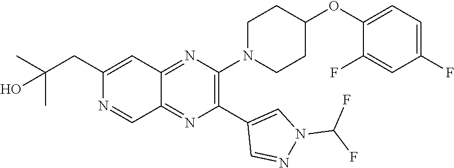 [SMILES: CC(C)(O)Cc1cc2nc(N3CCC(Oc4ccc(F)cc4F)CC3)c(-c3cnn(C(F)F)c3)nc2cn1] | 1-{3-[1-(difluoromethyl)-4- pyrazolyl]-2-[4-(2,4- difluorophenoxy)-1-piperidyl]- 1,4,6-triaza-7-naphthyl}-2-methyl- 2-propanol | NA |
| 701 | 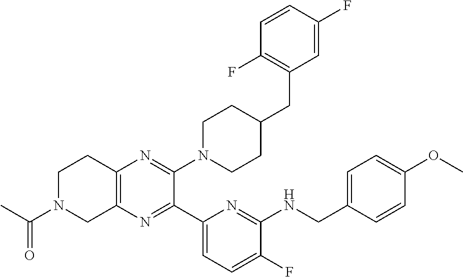 [SMILES: COc1ccc(CNc2nc(-c3nc4c(nc3N3CCC(Cc5cc(F)ccc5F)CC3)CCN(C(C)=O)C4)ccc2F)cc1] | 1-(2-{4-[(2,5- difluorophenyl)methyl]-1- piperidyl}-3-(5-fluoro-6-{ [(p- methoxyphenyl)methyl]amino}-2- pyridyl)-5,6,7,8-tetrahydro-1,4,6- triaza-6-naphthyl)-1-ethanone | NA |
| 702 | 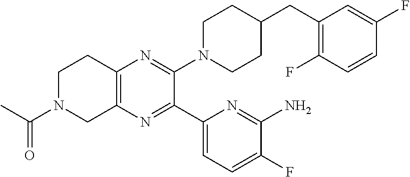 [SMILES: CC(=O)N1CCc2nc(N3CCC(Cc4cc(F)ccc4F)CC3)c(-c3ccc(F)c(N)n3)nc2C1] | 1-[3-(6-amino-5-fluoro-2-pyridyl)- 2-{4-[(2,5-difluorophenyl)methyl]- 1-piperidyl}-5,6,7,8-tetrahydro- 1,4,6-triaza-6-naphthyl]-1- ethanone | NA |
| 703 | 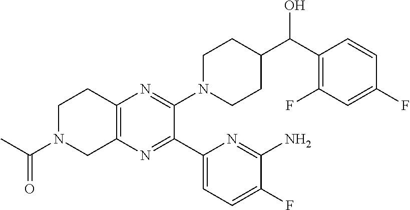 [SMILES: CC(=O)N1CCc2nc(N3CCC(C(O)c4ccc(F)cc4F)CC3)c(-c3ccc(F)c(N)n3)nc2C1] | 1-[3-(6-amino-5-fluoro-2-pyridyl)- 2-{4-[(2,4- difluorophenyl)hydroxymethyl]-1- piperidyl}-5,6,7,8-tetrahydro-1,4,6- triaza-6-naphthyl]-1-ethanone | NA |
| 704 | 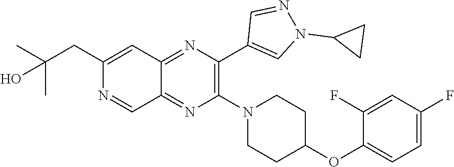 | 1-{2-(1-cyclopropyl-4-pyrazolyl)- 3-[4-(2,4-difluorophenoxy)-1- piperidyl]-1,4,6-triaza-7- naphthyl}-2-methyl-2-propanol | NA |
| 705 | 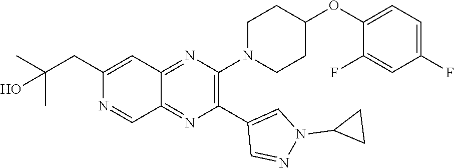 [SMILES: CC(C)(O)Cc1cc2nc(N3CCC(Oc4ccc(F)cc4F)CC3)c(-c3cnn(C4CC4)c3)nc2cn1] | 1-{3-(1-cyclopropyl-4-pyrazolyl)- 2-[4-(2,4-difluorophenoxy)-1- piperidyl]-1,4,6-triaza-7- naphthyl}-2-methyl-2-propanol | NA |
| 706 | 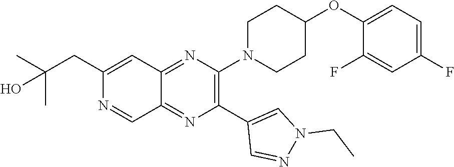 [SMILES: CCn1cc(-c2nc3cnc(CC(C)(C)O)cc3nc2N2CCC(Oc3ccc(F)cc3F)CC2)cn1] | 1-{2-[4-(2,4-difluorophenoxy)-1- piperidyl]-3-(1-ethyl-4-pyrazolyl)- 1,4,6-triaza-7-naphthyl}-2-methyl- 2-propanol | NA |
| 707 | 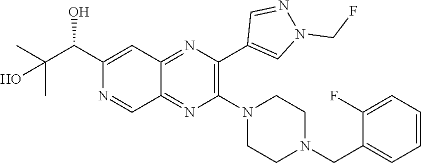 or enantiomer | (R)-1-{2-[1-(fluoromethyl)-4- pyrazolyl]-3-{4-[(o- fluorophenyl)methyl]-1- piperazinyl}-1,4,6-triaza-7- naphthyl}-2-methyl-1,2- propanediol, or enantiomer | not obtained |
| 708 | 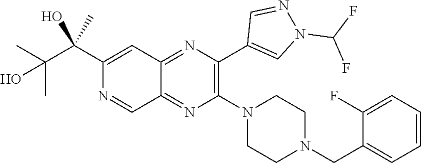 or enantiomer | (S)-2-{2-[1-(difluoromethyl)-4- pyrazolyl]-3-{4-[(o- fluorophenyl)methyl]-1- piperazinyl}-1,4,6-triaza-7- naphthyl}-3-methyl-2,3- butanediol, or enantiomer | (+) Isomer |
| 709 | 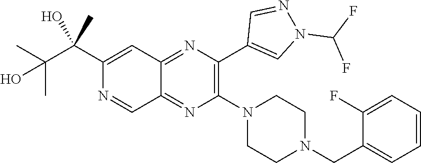 or enantiomer | (R)-2-{2-[1-(difluoromethyl)-4- pyrazolyl]-3-{4-[(o- fluorophenyl)methyl]-1- piperazinyl}-1,4,6-triaza-7- naphthyl}-3-methyl-2,3- butanediol, or enantiomer | (−) Isomer |
| 710 | 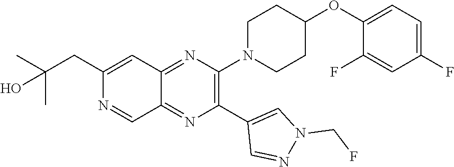 [SMILES: CC(C)(O)Cc1cc2nc(N3CCC(Oc4ccc(F)cc4F)CC3)c(-c3cnn(CF)c3)nc2cn1] | 1-{2-[4-(2,4-difluorophenoxy)-1- piperidyl]-3-[1-(fluoromethyl)-4- pyrazolyl]-1,4,6-triaza-7- naphthyl}-2-methyl-2-propanol | NA |
| 711 | 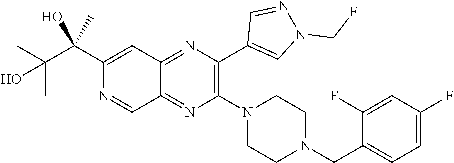 [SMILES: CC(C)(O)[C@@](C)(O)c1cc2nc(-c3cnn(CF)c3)c(N3CCN(Cc4ccc(F)cc4F)CC3)nc2cn1] or enantiomer | (S)-2-(3-{4-[(2,4- difluorophenyl)methyl]-1- piperazinyl}-2-[1-(fluoromethyl)- 4-pyrazolyl]-1,4,6-triaza-7- naphthyl)-3-methyl-2,3-butanediol, or enantiomer | (+) Isomer |
| 712 | 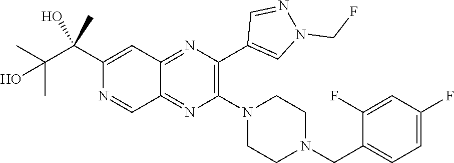 [SMILES: CC(C)(O)[C@](C)(O)c1cc2nc(-c3cnn(CF)c3)c(N3CCN(Cc4ccc(F)cc4F)CC3)nc2cn1] or enantiomer | (R)-2-(3-{4-[(2,4- difluorophenyl)methyl]-1- piperazinyl}-2-[1-(fluoromethyl)- 4-pyrazolyl]-1,4,6-triaza-7- naphthyl)-3-methyl-2,3-butanediol, or enantiomer | (−) Isomer |
| 713 | 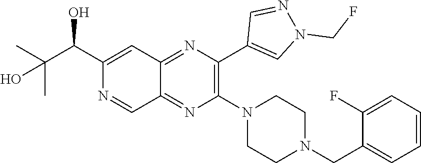 [SMILES: CC(C)(O)[C@@H](O)c1cc2nc(-c3cnn(CF)c3)c(N3CCN(Cc4ccccc4F)CC3)nc2cn1] or enantiomer | (S)-1-{2-[1-(fluoromethyl)-4- pyrazolyl]-3-{4-[(o- fluorophenyl)methyl]-1- piperazinyl}-1,4,6-triaza-7- naphthyl}-2-methyl-1,2- propanediol, or enantiomer | not obtained |
| 714 | 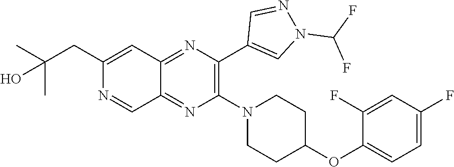 | 1-{2-[1-(difluoromethyl)-4- pyrazolyl]-3-[4-(2,4- difluorophenoxy)-1-piperidyl]- 1,4,6-triaza-7-naphthyl}-2-methyl- 2-propanol | NA |
| 715 | 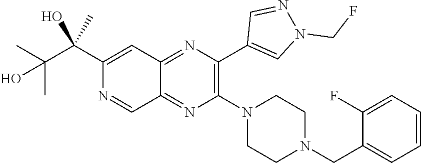 [SMILES: CC(C)(O)[C@@](C)(O)c1cc2nc(-c3cnn(CF)c3)c(N3CCN(Cc4ccccc4F)CC3)nc2cn1] or enantiomer | (S)-2-{2-[1-(fluoromethyl)-4- pyrazolyl]-3-{4-[(o- fluorophenyl)methyl]-1- piperazinyl}-1,4,6-triaza-7- naphthyl}-3-methyl-2,3- butanediol, or enantiomer | (+) Isomer |
| 716 | 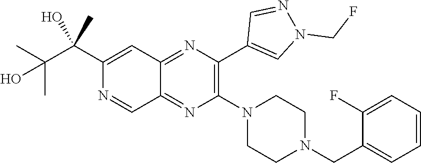 [SMILES: CC(C)(O)[C@](C)(O)c1cc2nc(-c3cnn(CF)c3)c(N3CCN(Cc4ccccc4F)CC3)nc2cn1] or enantiomer | (R)-2-{2-[1-(fluoromethyl)-4- pyrazolyl]-3-{4-[(o- fluorophenyl)methyl]-1- piperazinyl}-1,4,6-triaza-7- naphthyl}-3-methyl-2,3- butanediol, or enantiomer | (−) Isomer |
| 717 | 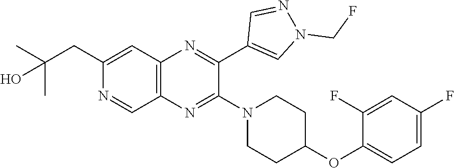 | 1-{3-[4-(2,4-difluorophenoxy)-1- piperidyl]-2-[1-(fluoromethyl)-4- pyrazolyl]-1,4,6-triaza-7- naphthyl}-2-methyl-2-propanol | NA |
| 718 | 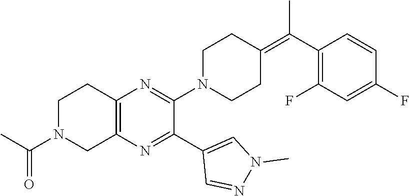 [SMILES: CC(=O)N1CCc2nc(N3CCC(=C(C)c4ccc(F)cc4F)CC3)c(-c3cnn(C)c3)nc2C1] | 1-(2-{4-[1-(2,4- difluorophenyl)ethylidene]-1- piperidyl}-3-(1-methyl-4- pyrazolyl)-5,6,7,8-tetrahydro- 1,4,6-triaza-6-naphthyl)-1- ethanone | NA |
| 719 | 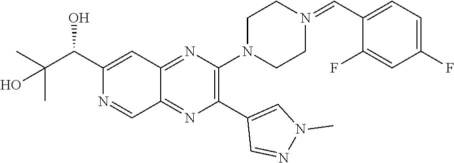 or enantiomer | (R)-1-(2-{4-[(2,4- difluorophenyl)methylene]-1- piperidyl}-3-(1-methyl-4- pyrazolyl)-1,4,6-triaza-7- naphthyl)-2-methyl-1,2- propanediol, or enantiomer | (+) Isomer |
| 720 | 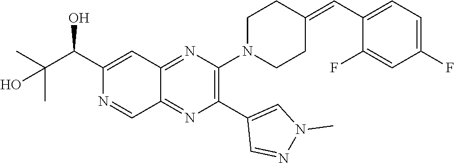 [SMILES: Cn1cc(-c2nc3cnc([C@H](O)C(C)(C)O)cc3nc2N2CCC(=Cc3ccc(F)cc3F)CC2)cn1] or enantiomer | (S)-1-(2-{4-[(2,4- difluorophenyl)methylene]-1- piperidyl}-3-(1-methyl-4- pyrazolyl)-1,4,6-triaza-7- naphthyl)-2-methyl-1,2- propanediol, or enantiomer | (−) Isomer |
| 721 | 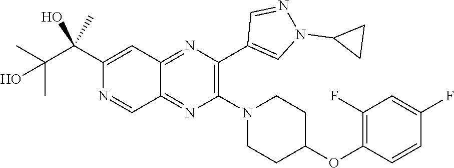 or enantiomer | (S)-2-{2-(1-cyclopropyl-4- pyrazolyl)-3-[4-(2,4- difluorophenoxy)-1-piperidyl]- 1,4,6-triaza-7-naphthyl}-3-methyl- 2,3-butanediol, or enantiomer | (+) Isomer |
| 722 | 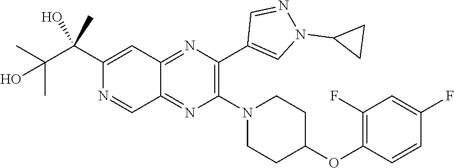 or enantiomer | (R)-2-{2-(1-cyclopropyl-4- pyrazolyl)-3-[4-(2,4- difluorophenoxy)-1-piperidyl]- 1,4,6-triaza-7-naphthyl}-3-methyl- 2,3-butanediol, or enantiomer | (−) Isomer |
| 723 | 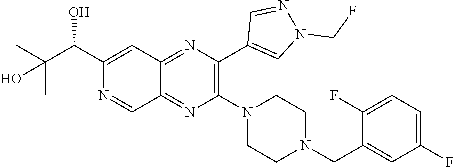 or enantiomer | (R)-1-(3-{4-[(2,5- difluorophenyl)methyl]-1- piperazinyl}-2-[1-(fluoromethyl)- 4-pyrazolyl]-1,4,6-triaza-7- naphthyl)-2-methyl-1,2- propanediol, or enantiomer | (+) Isomer |
| 724 | 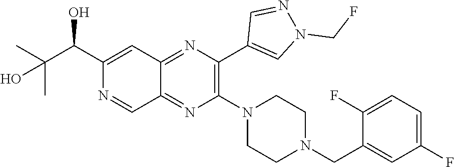 [SMILES: CC(C)(O)[C@@H](O)c1cc2nc(-c3cnn(CF)c3)c(N3CCN(Cc4cc(F)ccc4F)CC3)nc2cn1] or enantiomer | (S)-1-(3-{4-[(2,5- difluorophenyl)methyl]-1- piperazinyl}-2-[1-(fluoromethyl)- 4-pyrazolyl]-1,4,6-triaza-7- naphthyl)-2-methyl-1,2- propanediol, or enantiomer | (−) Isomer |
| 725 | 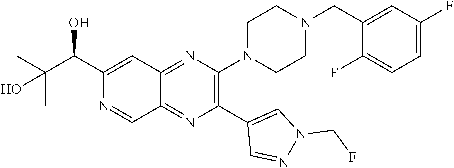 [SMILES: CC(C)(O)[C@@H](O)c1cc2nc(N3CCN(Cc4cc(F)ccc4F)CC3)c(-c3cnn(CF)c3)nc2cn1] or enantiomer | (S)-1-(2-{4-[(2,5- difluorophenyl)methyl]-1- piperazinyl}-3-[1-(fluoromethyl)- 4-pyrazolyl]-1,4,6-triaza-7- naphthyl)-2-methyl-1,2- propanediol, or enantiomer | not obtained |
| 726 | 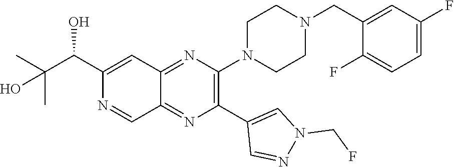 [SMILES: CC(C)(O)[C@H](O)c1cc2nc(N3CCN(Cc4cc(F)ccc4F)CC3)c(-c3cnn(CF)c3)nc2cn1] or enantiomer | (R)-1-(2-{4-[(2,5- difluorophenyl)methyl]-1- piperazinyl}-3-[1-(fluoromethyl)- 4-pyrazolyl]-1,4,6-triaza-7- naphthyl)-2-methyl-1,2- propanediol, or enantiomer | (−) Isomer |
| 727 | 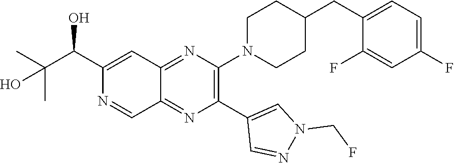 [SMILES: CC(C)(O)[C@@H](O)c1cc2nc(N3CCC(Cc4ccc(F)cc4F)CC3)c(-c3cnn(CF)c3)nc2cn1] or enantiomer | (S)-1-(2-{4-[(2,4- difluorophenyl)methyl]-1- piperidyl}-3-[1-(fluoromethyl)-4- pyrazolyl]-1,4,6-triaza-7- naphthyl)-2-methyl-1,2- propanediol, or enantiomer | (+) Isomer |
| 728 | 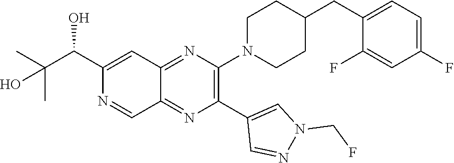 [SMILES: CC(C)(O)[C@H](O)c1cc2nc(N3CCC(Cc4ccc(F)cc4F)CC3)c(-c3cnn(CF)c3)nc2cn1] or enantiomer | (R)-1-(2-{4-[(2,4- difluorophenyl)methyl]-1- piperidyl}-3-[1-(fluoromethyl)-4- pyrazolyl]-1,4,6-triaza-7- naphthyl)-2-methyl-1,2- propanediol, or enantiomer | (−) Isomer |
| 729 | 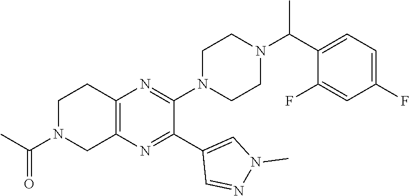 [SMILES: CC(=O)N1CCc2nc(N3CCN(C(C)c4ccc(F)cc4F)CC3)c(-c3cnn(C)c3)nc2C1] | 1-(2-{4-[1-(2,4- difluorophenyl)ethyl]-1- piperazinyl}-3-(1-methyl-4- pyrazolyl)-5,6,7,8-tetrahydro- 1,4,6-triaza-6-naphthyl)-1- ethanone | NA |
| 730 | 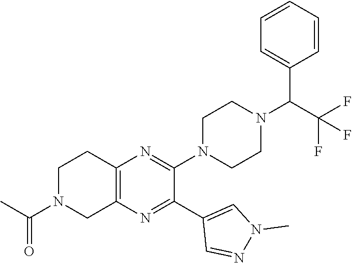 [SMILES: CC(=O)N1CCc2nc(N3CCN(C(c4ccccc4)C(F)(F)F)CC3)c(-c3cnn(C)c3)nc2C1] | 1-{3-(1-methyl-4-pyrazolyl)-2-[4- (2,2,2-trifluoro-1-phenylethyl)-1- piperazinyl]-5,6,7,8-tetrahydro- 1,4,6-triaza-6-naphthyl}-1- ethanone | NA |
| 731 | 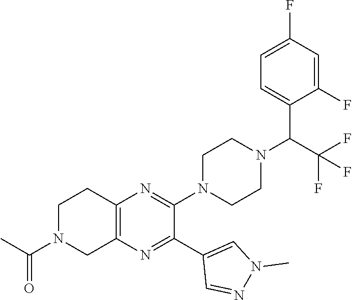 [SMILES: CC(=O)N1CCc2nc(N3CCN(C(c4ccc(F)cc4F)C(F)(F)F)CC3)c(-c3cnn(C)c3)nc2C1] | 1-(2-{4-[1-(2,4-difluorophenyl)- 2,2,2-trifluoroethyl]-1- piperazinyl}-3-(1-methyl-4- pyrazolyl)-5,6,7,8-tetrahydro- 1,4,6-triaza-6-naphthyl)-1- ethanone | NA |
| 732 | 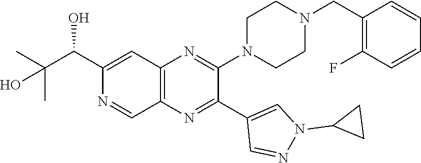 [SMILES: CC(C)(O)[C@H](O)c1cc2nc(N3CCN(Cc4ccccc4F)CC3)c(-c3cnn(C4CC4)c3)nc2cn1] or enantiomer | (R)-1-[3-(1-cyclopropyl-4- pyrazolyl)-2-{4-[(o- fluorophenyl)methyl]-1- piperazinyl}-1,4,6-triaza-7- naphthyl]-2-methyl-1,2- propanediol, or enantiomer | (+) Isomer |
| 733 | 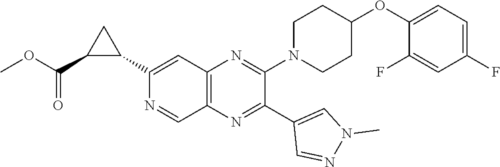 [SMILES: COC(=O)[C@H]1C[C@@H]1c1cc2nc(N3CCC(Oc4ccc(F)cc4F)CC3)c(-c3cnn(C)c3)nc2cn1] or enantiomer | methyl (1S,2S)-2-{2-[4-(2,4- difluorophenoxy)-1-piperidyl]-3- (1-methyl-4-pyrazolyl)-1,4,6- triaza-7- naphthyl}cyclopropanecarboxylate, or enantiomer | NA |
| 734 | 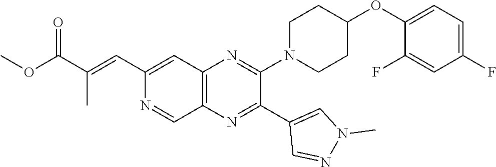 [SMILES: COC(=O)/C(C)=C/c1cc2nc(N3CCC(Oc4ccc(F)cc4F)CC3)c(-c3cnn(C)c3)nc2cn1] | methyl 3-{2-[4-(2,4- difluorophenoxy)-1-piperidyl]-3- (1-methyl-4-pyrazolyl)-1,4,6- triaza-7-naphthyl}methacrylate | NA |
| 735 | 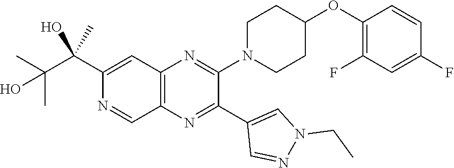 [SMILES: CCn1cc(-c2nc3cnc([C@](C)(O)C(C)(C)O)cc3nc2N2CCC(Oc3ccc(F)cc3F)CC2)cn1] or enantiomer | (S)-2-{2-[4-(2,4-difluorophenoxy)- 1-piperidyl]-3-(1-ethyl-4- pyrazolyl)-1,4,6-triaza-7- naphthyl}-3-methyl-2,3- butanediol, or enantiomer | not obtained |
| 736 | 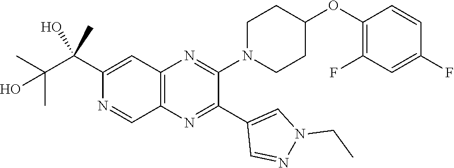 [SMILES: CCn1cc(-c2nc3cnc([C@@](C)(O)C(C)(C)O)cc3nc2N2CCC(Oc3ccc(F)cc3F)CC2)cn1] or enantiomer | (R)-2-{2-[4-(2,4-difluorophenoxy)- 1-piperidyl]-3-(1-ethyl-4- pyrazolyl)-1,4,6-triaza-7- naphthyl}-3-methyl-2,3- butanediol, or enantiomer | not obtained |
| 737 | 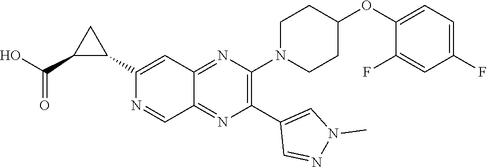 [SMILES: Cn1cc(-c2nc3cnc([C@H]4C[C@@H]4C(=O)O)cc3nc2N2CCC(Oc3ccc(F)cc3F)CC2)cn1] , or enantiomer | (1S,2S)-2-{2-[4-(2,4- difluorophenoxy)-1-piperidyl]-3- (1-methyl-4-pyrazolyl)-1,4,6- triaza-7- naphthyl} cyclopropanecarboxylic acid, or enantiomer | NA |
| 738 | 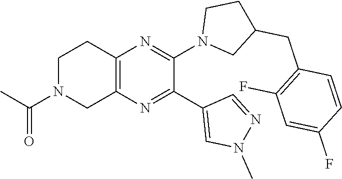 [SMILES: CC(=O)N1CCc2nc(N3CCC(Cc4ccc(F)cc4F)C3)c(-c3cnn(C)c3)nc2C1] | 1-(2-{3-[(2,4- difluorophenyl)methyl]-1- pyrrolidinyl}-3-(1-methyl-4- pyrazolyl)-5,6,7,8-tetrahydro- 1,4,6-triaza-6-naphthyl)-1- ethanone | NA |
| 739 | 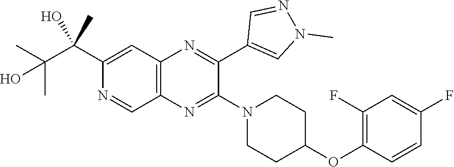 or enantiomer | (R)-2-{3-[4-(2,4-difluorophenoxy)- 1-piperidyl]-2-(1-methyl-4- pyrazolyl)-1,4,6-triaza-7- naphthyl}-3-methyl-2,3-butanediol | (−) Isomer |
| 740 | 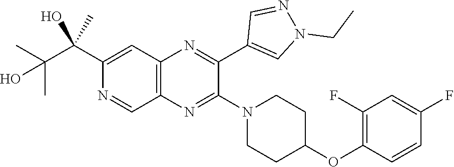 or enantiomer | (S)-2-{3-[4-(2,4-difluorophenoxy)- 1-piperidyl]-2-(1-ethyl-4- pyrazolyl)-1,4,6-triaza-7- naphthyl}-3-methyl-2,3- butanediol, or enantiomer | (+) Isomer |
| 741 | 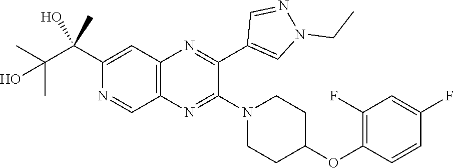 or enantiomer | (R)-2-{3-[4-(2,4-difluorophenoxy)- 1-piperidyl]-2-(1-ethyl-4- pyrazolyl)-1,4,6-triaza-7- naphthyl}-3-methyl-2,3- butanediol, or enantiomer | (−) Isomer |
| 742 | 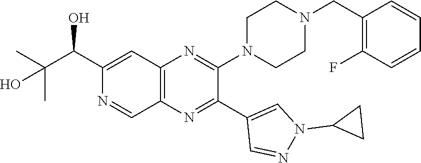 [SMILES: CC(C)(O)[C@@H](O)c1cc2nc(N3CCN(Cc4ccccc4F)CC3)c(-c3cnn(C4CC4)c3)nc2cn1] or enantiomer | (S)-1-[3-(1-cyclopropyl-4- pyrazolyl)-2-{4-[(o- fluorophenyl)methyl]-1- piperazinyl}-1,4,6-triaza-7- naphthyl]-2-methyl-1,2- propanediol, or enantiomer | (−) Isomer |
| 743 | 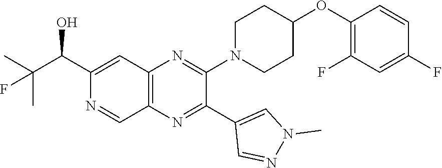 [SMILES: Cn1cc(-c2nc3cnc([C@H](O)C(C)(C)F)cc3nc2N2CCC(Oc3ccc(F)cc3F)CC2)cn1] or enantiomer | (S)-1-{2-[4-(2,4-difluorophenoxy)- 1-piperidyl]-3-(1-methyl-4- pyrazolyl)-1,4,6-triaza-7- naphthyl}-2-fluoro-2-methyl-1- propanol, or enantiomer | not obtained |
| 744 | 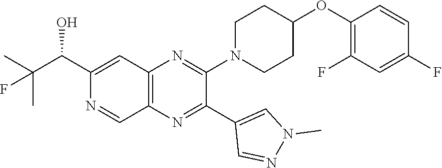 [SMILES: Cn1cc(-c2nc3cnc([C@@H](O)C(C)(C)F)cc3nc2N2CCC(Oc3ccc(F)cc3F)CC2)cn1] , or enantiomer | (R)-1-{2-[4-(2,4-difluorophenoxy)- 1-piperidyl]-3-(1-methyl-4- pyrazolyl)-1,4,6-triaza-7- naphthyl}-2-fluoro-2-methyl-1- propanol, or enantiomer | not obtained |
| 745 | 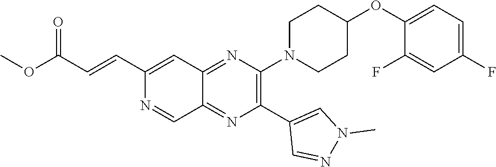 [SMILES: COC(=O)/C=C/c1cc2nc(N3CCC(Oc4ccc(F)cc4F)CC3)c(-c3cnn(C)c3)nc2cn1] | methyl 3-{2-[4-(2,4- difluorophenoxy)-1-piperidyl]-3- (1-methyl-4-pyrazolyl)-1,4,6- triaza-7-naphthyl}acrylate | NA |
| 746 | 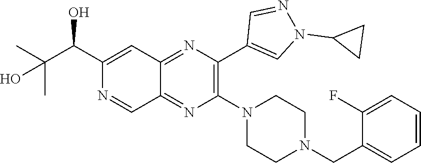 [SMILES: CC(C)(O)[C@@H](O)c1cc2nc(-c3cnn(C4CC4)c3)c(N3CCN(Cc4ccccc4F)CC3)nc2cn1] , or enantiomer | (S)-1-[2-(1-cyclopropyl-4- pyrazolyl)-3-{4-[(o- fluorophenyl)methyl]-1- piperazinyl}-1,4,6-triaza-7- naphthyl]-2-methyl-1,2- propanediol, or enantiomer | (+) Isomer |
| 747 | 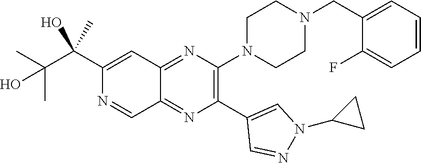 [SMILES: CC(C)(O)[C@@](C)(O)c1cc2nc(N3CCN(Cc4ccccc4F)CC3)c(-c3cnn(C4CC4)c3)nc2cn1] or enantiomer | (S)-2-[3-(1-cyclopropyl-4- pyrazolyl)-2-{4-[(o- fluorophenyl)methyl]-1- piperazinyl}-1,4,6-triaza-7- naphthyl]-3-methyl-2,3-butanediol | (−) Isomer |
| 748 | 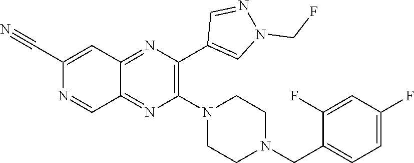 [SMILES: N#Cc1cc2nc(-c3cnn(CF)c3)c(N3CCN(Cc4ccc(F)cc4F)CC3)nc2cn1] | 3-{4-[(2,4-difluorophenyl)methyl]-1- piperazinyl}-2-[1-(fluoromethyl)-4- pyrazolyl]-1,4,6-triaza-7- naphthonitrile | NA |
| 749 | 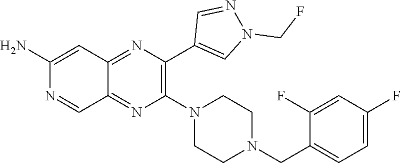 [SMILES: Nc1cc2nc(-c3cnn(CF)c3)c(N3CCN(Cc4ccc(F)cc4F)CC3)nc2cn1] | 3-{4-[(2,4-difluorophenyl)methyl]-1- piperazinyl}-2-[1-(fluoromethyl)-4- pyrazolyl]-1,4,6-triaza-7- naphthylamine | NA |
| 750 | 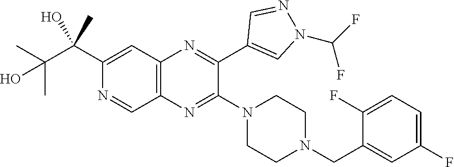 , or enantiomer | 1-2-{2-[1-(difluoromethyl)-4- pyrazolyl]-3-{4-[(2,5- difluorophenyl)methyl]-1- piperazinyl}-1,4,6-triaza-7-naphthyl}- 3-methyl-2,3-butanediol, or enantiomer | (−) Isomer |
| 751 | 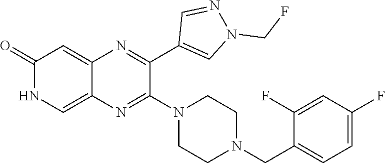 [SMILES: O=c1cc2nc(-c3cnn(CF)c3)c(N3CCN(Cc4ccc(F)cc4F)CC3)nc2c[nH]1] | 3-{4-[(2,4-difluorophenyl)methyl]-1- piperazinyl}-2-[1-(fluoromethyl)-4- pyrazolyl]-1,4,6-triaza-7(6H)- naphthalenone | NA |
| 752 | 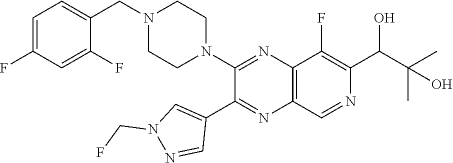 [SMILES: CC(C)(O)C(O)c1ncc2nc(-c3cnn(CF)c3)c(N3CCN(Cc4ccc(F)cc4F)CC3)nc2c1F] | 1-(2-{4-[(2,4-difluorophenyl)methyl]- 1-piperazinyl}-8-fluoro-3-[1- (fluoromethyl)-4-pyrazolyl]-1,4,6- triaza-7-naphthyl)-2-methyl-1,2- propanediol | Racemate |
| 753 | 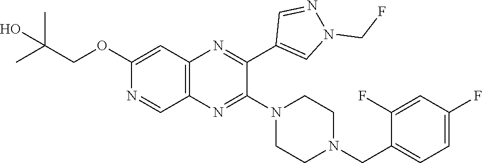 [SMILES: CC(C)(O)COc1cc2nc(-c3cnn(CF)c3)c(N3CCN(Cc4ccc(F)cc4F)CC3)nc2cn1] | 1-(3-{4-[(2,4-difluorophenyl)methyl]- 1-piperazinyl}-2-[1-(fluoromethyl)-4- pyrazolyl]-1,4,6-triaza-7- naphthyloxy)-2-methyl-2-propanol | NA |
| 754 | 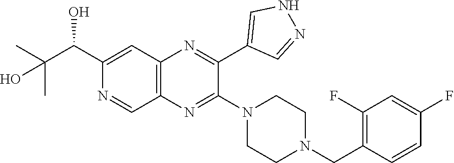 [SMILES: CC(C)(O)[C@H](O)c1cc2nc(-c3cn[nH]c3)c(N3CCN(Cc4ccc(F)cc4F)CC3)nc2cn1] , or enantiomer | (R)-1-(3-{4-[(2,4- difluorophenyl)methyl]-1- piperazinyl}-2-(4-pyrazolyl)-1,4,6- triaza-7-naphthyl)-2-methyl-1,2- propanediol, or enantiomer | (−) Isomer |
| 755 | 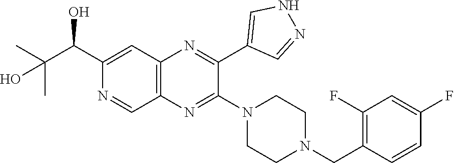 [SMILES: CC(C)(O)[C@@H](O)c1cc2nc(-c3cn[nH]c3)c(N3CCN(Cc4ccc(F)cc4F)CC3)nc2cn1] , or enantiomer | (S)-1-(3-{4-[(2,4- difluorophenyl)methyl]-1- piperazinyl}-2-(4-pyrazolyl)-1,4,6- triaza-7-naphthyl)-2-methyl-1,2- propanediol, or enantiomer | (+) Isomer |
| 756 | 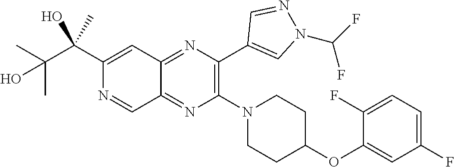 , or enantiomer | (S)-2-{2-[1-(difluoromethyl)-4- pyrazolyl]-3-{4-[(2,5- difluorophenyl)methyl]-1- piperazinyl}-1,4,6-triaza-7-naphthyl}- 3-methyl-2,3-butanediol, or enantiomer | (+) Isomer |
| 757 | 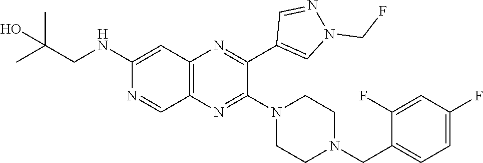 [SMILES: CC(C)(O)CNc1cc2nc(-c3cnn(CF)c3)c(N3CCN(Cc4ccc(F)cc4F)CC3)nc2cn1] | 1-(3-{4-[(2,4-difluorophenyl)methyl]- 1-piperazinyl}-2-[1-(fluoromethyl)-4- pyrazolyl]-1,4,6-triaza-7- naphthylamino)-2-methyl-2-propanol | NA |
| 758 | 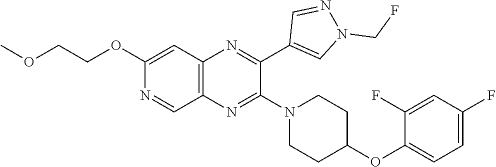 | 3-{4-[(2,4-difluorophenyl)methyl]-1- piperazinyl}-2-[1-(fluoromethyl)-4- pyrazolyl]-7-(2-methoxyethoxy)- 1,4,6-triazanaphthalene | NA |
| 759 | 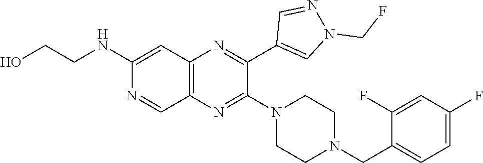 [SMILES: OCCNc1cc2nc(-c3cnn(CF)c3)c(N3CCN(Cc4ccc(F)cc4F)CC3)nc2cn1] | 2-(3-{4-[(2,4-difluorophenyl)methyl]- 1-piperazinyl}-2-[1-(fluoromethyl)-4- pyrazolyl]-1,4,6-triaza-7- naphthylamino)ethanol | NA |
| 760 | 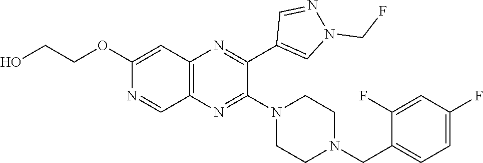 [SMILES: OCCOc1cc2nc(-c3cnn(CF)c3)c(N3CCN(Cc4ccc(F)cc4F)CC3)nc2cn1] | 2-(3-{4-[(2,4-difluorophenyl)methyl]- 1-piperazinyl}-2-[1-(fluoromethyl)-4- pyrazolyl]-1,4,6-triaza-7- naphthyloxy)ethanol | NA |
| 761 | 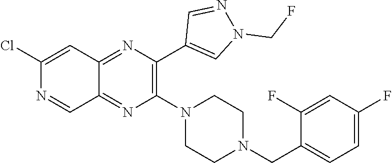 [SMILES: FCn1cc(-c2nc3cc(Cl)ncc3nc2N2CCN(Cc3ccc(F)cc3F)CC2)cn1] | 7-chloro-3-{4-[(2,4- difluorophenyl)methyl]-1- piperazinyl}-2-[1-(fluoromethyl)-4- pyrazolyl]-1,4,6-triazanaphthalene | NA |
| 762 | 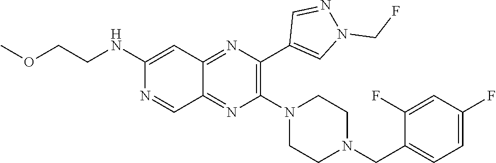 [SMILES: COCCNc1cc2nc(-c3cnn(CF)c3)c(N3CCN(Cc4ccc(F)cc4F)CC3)nc2cn1] | (2-methoxyethyl)(3-{4-[(2,4- difluorophenyl)methyl]-1- piperazinyl}-2-[1-(fluoromethyl)-4- pyrazolyl]-1,4,6-triaza-7- naphthyl)amine | NA |
| 763 | 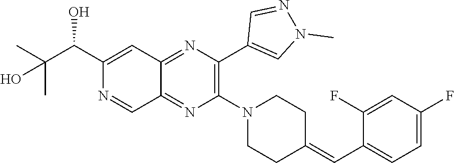 [SMILES: Cn1cc(-c2nc3cc([C@@H](O)C(C)(C)O)ncc3nc2N2CCC(=Cc3ccc(F)cc3F)CC2)cn1] , or enantIer | (R)-1-(3-{4-[(2,4- difluorophenyl)methylene]-1- piperidyl}-2-(1-methyl-4-pyrazolyl)- 1,4,6-triaza-7-naphthyl)-2-methyl-1,2- propanediol, or enantiomer | (−) Isomer |
| 764 | 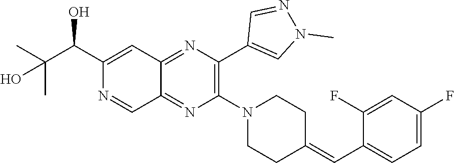 [SMILES: Cn1cc(-c2nc3cc([C@H](O)C(C)(C)O)ncc3nc2N2CCC(=Cc3ccc(F)cc3F)CC2)cn1] or enantiomer | (S)-1-(3-{4-[(2,4- difluorophenyl)methylene]-1- piperidyl}-2-(1-methyl-4-pyrazolyl)- 1,4,6-triaza-7-naphthyl)-2-methyl-1,2- propanediol, or enantiomer | (+) Isomer |
| 765 | 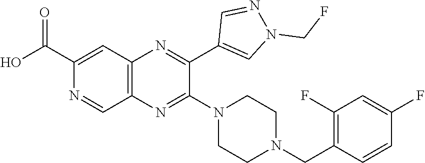 [SMILES: O=C(O)c1cc2nc(-c3cnn(CF)c3)c(N3CCN(Cc4ccc(F)cc4F)CC3)nc2cn1] | 3-{4-[(2,4-difluorophenyl)methyl]-1- piperazinyl}-2-[1-(fluoromethyl)-4- pyrazolyl]-1,4,6-triaza-7-naphthoic acid | NA |
| 766 | 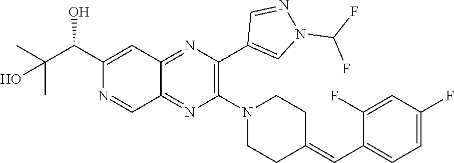 [SMILES: CC(C)(O)[C@H](O)c1cc2nc(-c3cnn(C(F)F)c3)c(N3CCC(=Cc4ccc(F)cc4F)CC3)nc2cn1] , or enliomer | (R)-1-{2-[1-(difluoromethyl)-4- pyrazolyl]-3-{4-[(2,4- difluorophenyl)methylene]-1- piperidyl}-1,4,6-triaza-7-naphthyl}-2- methyl-1,2-propanediol, or enantiomer | (+) Isomer |
| 767 | 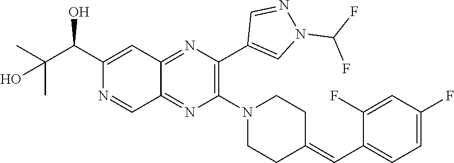 [SMILES: CC(C)(O)[C@@H](O)c1cc2nc(-c3cnn(C(F)F)c3)c(N3CCC(=Cc4ccc(F)cc4F)CC3)nc2cn1] , or enantiomer | (S)-1-{2-[1-(difluoromethyl)-4- pyrazolyl]-3-{4-[(2,4- difluorophenyl)methylene]-1- piperidyl}-1,4,6-triaza-7-naphthyl}-2- methyl-1,2-propanediol, or enantiomer | (−) Isomer |
| 768 | 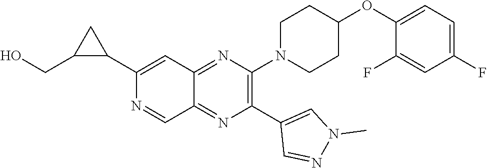 [SMILES: Cn1cc(-c2nc3cnc(C4CC4CO)cc3nc2N2CCC(Oc3ccc(F)cc3F)CC2)cn1] | (2-{2-[4-(2,4-difluorophenoxy)-1- piperidyl]-3-(1-methyl-4-pyrazolyl)- 1,4,6-triaza-7- naphthyl}cyclopropyl)methanol | NA |
| 769 | 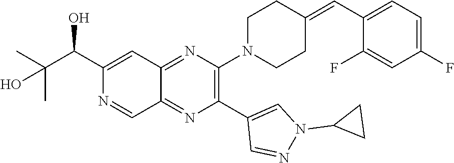 [SMILES: CC(C)(O)[C@@H](O)c1cc2nc(N3CCC(=Cc4ccc(F)cc4F)CC3)c(-c3cnn(C4CC4)c3)nc2cn1] , or enantiomer | (S)-1-[3-(1-cyclopropyl-4-pyrazolyl)- 2-{4-[(2,4-difluorophenyl)methylene]- 1-piperidyl}-1,4,6-triaza-7-naphthyl]- 2-methyl-1,2-propanediol, or enantiomer | (+) Isomer |
| 770 | 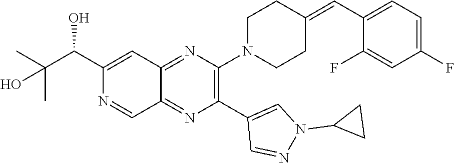 [SMILES: CC(C)(O)[C@H](O)c1cc2nc(N3CCC(=Cc4ccc(F)cc4F)CC3)c(-c3cnn(C4CC4)c3)nc2cn1] , orIantiomer | (R)-1-[3-(1-cyclopropyl-4-pyrazolyl)- 2-{4-[(2,4-difluorophenyl)methylene]- 1-piperidyl}-1,4,6-triaza-7-naphthyl]- 2-methyl-1,2-propanediol, or enantiomer | (−) Isomer |
| 771 | 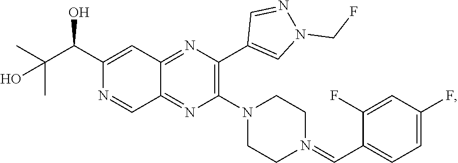 or enantiomer | (S)-1-(3-{4-[(2,4- difluorophenyl)methylene]-1- piperidyl}-2-[1-(fluoromethyl)-4- pyrazolyl]-1,4,6-triaza-7-naphthyl)-2- methyl-1,2-propanediol, or enantiomer | (−) Isomer |
| 772 | 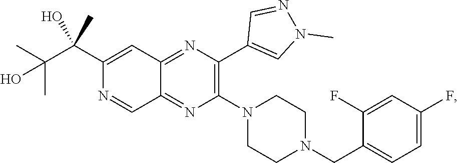 I enantiomer | (R)-2-(3-{4-[(2,4- difluorophenyl)methyl]-1- piperazinyl}-2-(1-methyl-4- pyrazolyl)-1,4,6-triaza-7-naphthyl)-3- methyl-2,3-butanediol | (−) Isomer |
| 773 | 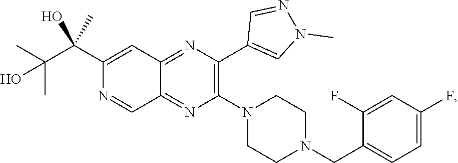 or enantiomer | (S)-2-(3-{4-[(2,4- difluorophenyl)methyl]-1- piperazinyl}-2-(1-methyl-4- pyrazolyl)-1,4,6-triaza-7-naphthyl)-3- methyl-2,3-butanediol | (+) Isomer |
| 774 | 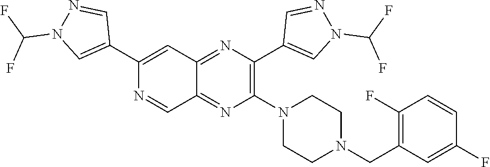 [SMILES: Fc1ccc(F)c(CN2CCN(c3nc4cnc(-c5cnn(C(F)F)c5)cc4nc3-c3cnn(C(F)F)c3)CC2)c1] | 2,7-bis [1-(difluoromethyl)-4- pyrazolyl]-3-{4-[(2,5- difluorophenyl)methyl]-1- piperazinyl}-1,4,6-triazanaphthalene | NA |
| 775 | 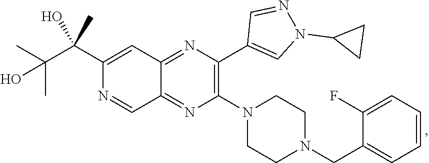 [SMILES: CC(C)(O)[C@](C)(O)c1cc2nc(-c3cnn(C4CC4)c3)c(N3CCN(Cc4ccccc4F)CC3)nc2cn1] or enantiomer | (R)-2-[2-(1-cyclopropyl-4-pyrazolyl)- 3-{4-[(o-fluorophenyl)methyl]-1- piperazinyl}-1,4,6-triaza-7-naphthyl]- 3-methyl-2,3-butanediol | (−) Isomer |
| 776 | 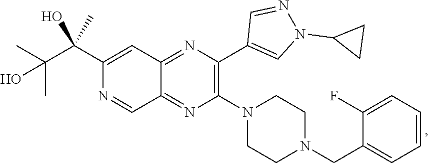 [SMILES: CC(C)(O)[C@@](C)(O)c1cc2nc(-c3cnn(C4CC4)c3)c(N3CCN(Cc4ccccc4F)CC3)nc2cn1] or enantiomer | (S)-2-[2-(1-cyclopropyl-4-pyrazolyl)- 3-{4-[(o-fluorophenyl)methyl]-1- piperazinyl}-1,4,6-triaza-7-naphthyl]- 3-methyl-2,3-butanediol, or enantiomer | (+) Isomer |
| 777 | 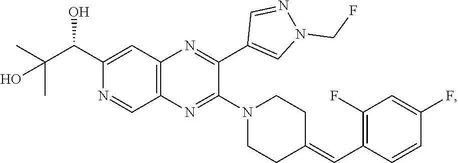 or enantiomer | (R)-1-(3-{4-[(2,4- difluorophenyl)methylene]-1- piperidyl}-2-[1-(fluoromethyl)-4- pyrazolyl]-1,4,6-triaza-7-naphthyl)-2- methyl-1,2-propanediol, or enantiomer | (+) Isomer |
| 778 | 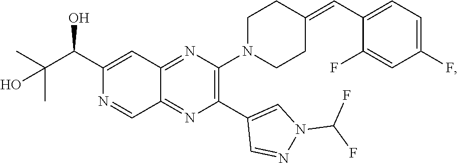 [SMILES: CC(C)(O)[C@@H](O)c1cc2nc(N3CCC(=Cc4ccc(F)cc4F)CC3)c(-c3cnn(C(F)F)c3)nc2cn1] or enantiomer | (S)-1-{3-[1-(difluoromethyl)-4- pyrazolyl]-2-{4-[(2,4- difluorophenyl)methylene]-1- piperidyl}-1,4,6-triaza-7-naphthyl}-2- methyl-1,2-propanediol, or enantiomer | (+) Isomer |
| 779 | 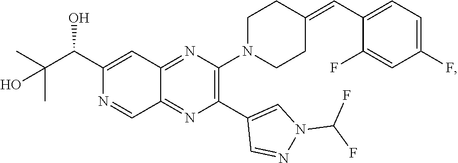 [SMILES: CC(C)(O)[C@H](O)c1cc2nc(N3CCC(=Cc4ccc(F)cc4F)CC3)c(-c3cnn(C(F)F)c3)nc2cn1] or enantiomer | (R)-1-{3-[1-(difluoromethyl)-4- pyrazolyl]-2-{4-[(2,4- difluorophenyl)methylene]-1- piperidyl}-1,4,6-triaza-7-naphthyl}-2- methyl-1,2-propanediol, or enantiomer | (−) Isomer |
| 780 | 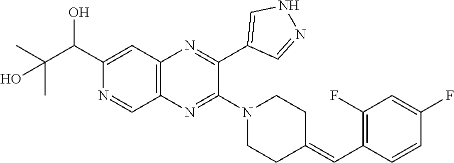 [SMILES: CC(C)(O)C(O)c1cc2nc(-c3cn[nH]c3)c(N3CCC(=Cc4ccc(F)cc4F)CC3)nc2cn1] | 1-(3-{4-[(2,4- difluorophenyl)methylene]-1- piperidyl}-2-(4-pyrazolyl)-1,4,6- triaza-7-naphthyl)-2-methyl-1,2- propanediol | NA |
| 781 | 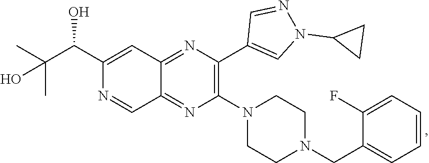 [SMILES: CC(C)(O)[C@H](O)c1cc2nc(-c3cnn(C4CC4)c3)c(N3CCN(Cc4ccccc4F)CC3)nc2cn1] or enantiomer | (R)-1-[2-(1-cyclopropyl-4-pyrazolyl)- 3-{4-[(o-fluorophenyl)methyl]-1- piperazinyl}-1,4,6-triaza-7-naphthyl]- 2-methyl-1,2-propanediol, or enantiomer | (−) Isomer |
| 782 | 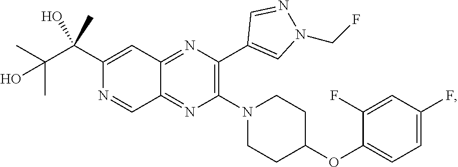 or enantiomer | (R)-2-{3-[4-(2,4-difluorophenoxy)-1- piperidyl]-2-[1-(fluoromethyl)-4- pyrazolyl]-1,4,6-triaza-7-naphthyl}-3- methyl-2,3-butanediol, or enantiomer | (+) Isomer |
| 783 | 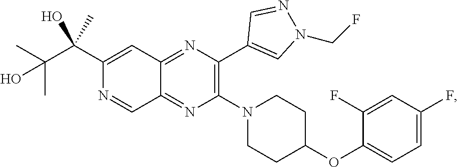 or enantiomer | (S)-2-{3-[4-(2,4-difluorophenoxy)-1- piperidyl]-2-[1-(fluoromethyl)-4- pyrazolyl]-1,4,6-triaza-7-naphthyl}-3- methyl-2,3-butanediol, or enantiomer | (−) Isomer |
| 784 | 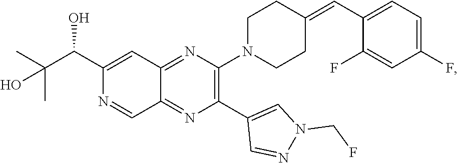 [SMILES: CC(C)(O)[C@H](O)c1cc2nc(N3CCC(=Cc4ccc(F)cc4F)CC3)c(-c3cnn(CF)c3)nc2cn1] or enantiomer | (R)-1-(2-{4-[(2,4- difluorophenyl)methylene]-1- piperidyl}-3-[1-(fluoromethyl)-4- pyrazolyl]-1,4,6-triaza-7-naphthyl)-2- methyl-1,2-propanediol, or enantiomer | (−) Isomer |
| 785 | 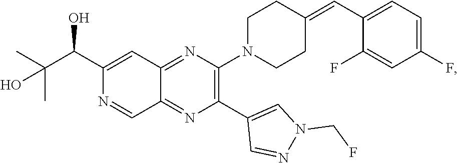 [SMILES: CC(C)(O)[C@@H](O)c1cc2nc(N3CCC(=Cc4ccc(F)cc4F)CC3)c(-c3cnn(CF)c3)nc2cn1] or enantiomer | (S)-1-(2-{4-[(2,4- difluorophenyl)methylene]-1- piperidyl}-3-[1-(fluoromethyl)-4- pyrazolyl]-1,4,6-triaza-7-naphthyl)-2- methyl-1,2-propanediol, or enantiomer | (+) Isomer |
| 786 | 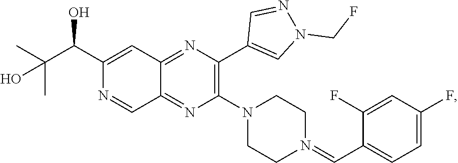 or enantiomer | (S)-1-(3-{4-[(2,4- difluorophenyl)methyl]-1-piperidyl}- 2-[1-(fluoromethyl)-4-pyrazolyl]- 1,4,6-triaza-7-naphthyl)-2-methyl-1,2- propanediol, or enantiomer | (−) Isomer |
| 787 | 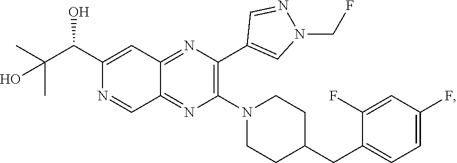 [SMILES: CC(C)(O)[C@H](O)c1cc2nc(-c3cnn(CF)c3)c(N3CCC(Cc4ccc(F)cc4F)CC3)nc2cn1] or enantiomer | (R)-1-(3-{4-[(2,4- difluorophenyl)methyl]-1-piperidyl}- 2-[1-(fluoromethyl)-4-pyrazolyl]- 1,4,6-triaza-7-naphthyl)-2-methyl-1,2- propanediol, or enantiomer | (+) Isomer |
| 788 | 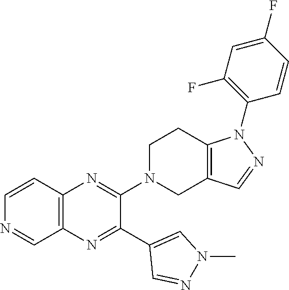 [SMILES: Cn1cc(-c2nc3cnccc3nc2N2CCc3c(cnn3-c3ccc(F)cc3F)C2)cn1] | 1-(2,4-difluorophenyl)-5-[3-(1- methyl-4-pyrazolyl)-1,4,6-triaza-2- naphthyl]-4,5,6,7-tetrahydro-1H-1,2,5- triazaindene | NA |
| 789 | 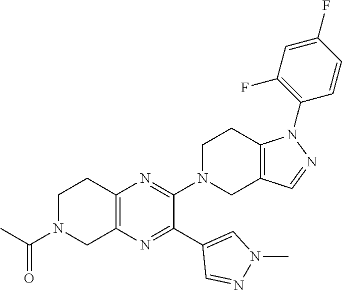 [SMILES: CC(=O)N1CCc2nc(N3CCc4c(cnn4-c4ccc(F)cc4F)C3)c(-c3cnn(C)c3)nc2C1] | 1-{2-[1-(2,4-difluorophenyl)-4,5,6,7- tetrahydro-1H-1,2,5-triazainden-5-yl]- 3-(1-methyl-4-pyrazolyl)-5,6,7,8- tetrahydro-1,4,6-triaza-6-naphthyl}-1- ethanone | NA |
| 790 | 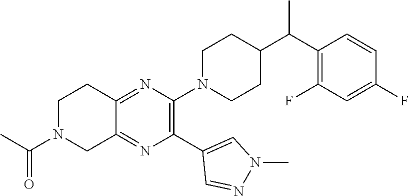 [SMILES: CC(=O)N1CCc2nc(N3CCC(C(C)c4ccc(F)cc4F)CC3)c(-c3cnn(C)c3)nc2C1] | 1-(2-{4-[1-(2,4-difluorophenyl)ethyl]- 1-piperidyl}-3-(1-methyl-4- pyrazolyl)-5,6,7,8-tetrahydro-1,4,6- triaza-6-naphthyl)-1-ethanone | NA |
| 791 | 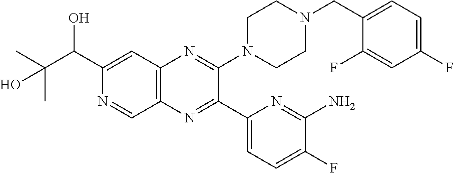 [SMILES: CC(C)(O)C(O)c1cc2nc(N3CCN(Cc4ccc(F)cc4F)CC3)c(-c3ccc(F)c(N)n3)nc2cn1] | 1-[3-(6-amino-5-fluoro-2-pyridyl)-2- {4-[(2,4-difluorophenyl)methyl]-1- piperazinyl}-1,4,6-triaza-7-naphthyl]- 2-methyl-1,2-propanediol | NA |
| 792 | 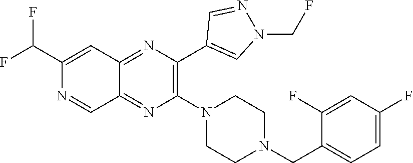 [SMILES: FCn1cc(-c2nc3cc(C(F)F)ncc3nc2N2CCN(Cc3ccc(F)cc3F)CC2)cn1] | 7-(difluoromethyl)-3-{4-[(2,4- difluorophenyl)methyl]-1- piperazinyl}-2-[1-(fluoromethyl)-4- pyrazolyl]-1,4,6-triazanaphthalene | NA |
| 793 | 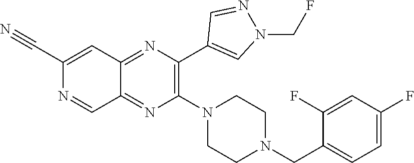 [SMILES: N#Cc1cc2nc(-c3cnn(CF)c3)c(N3CCN(Cc4ccc(F)cc4F)CC3)nc2cn1] | 3-{4-[(2,4-difluorophenyl)methyl]-1- piperazinyl}-2-[1-(fluoromethyl)-4- pyrazolyl]-1,4,6-triaza-7- naphthonitrile | NA |
| 794 | 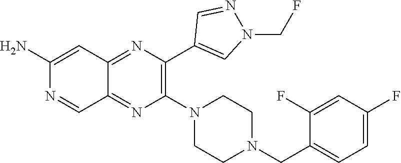 [SMILES: Nc1cc2nc(-c3cnn(CF)c3)c(N3CCN(Cc4ccc(F)cc4F)CC3)nc2cn1] | 3-{4-[(2,4-difluorophenyl)methyl]-1- piperazinyl}-2-[1-(fluoromethyl)-4- pyrazolyl]-1,4,6-triaza-7- naphthylamine | NA |
| 795 | 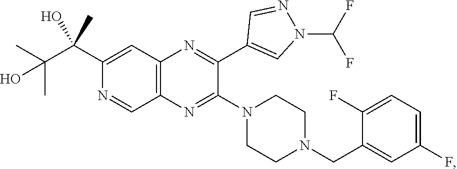 [SMILES: CC(C)(O)[C@](C)(O)c1cc2nc(-c3cnn(C(F)F)c3)c(N3CCN(Cc4cc(F)ccc4F)CC3)nc2cn1] or enantiomer | (R)-2-{2-[1- (difluoromethyl)-4- pyrazolyl]-3-{4- [(2,5-difluorophenyl)methyl]- 1-piperazinyl}-1,4,6-triaza-7- naphthyl}-3-methyl-2,3- butanediol, orenantiomer | (−) Isomer |
| 796 | 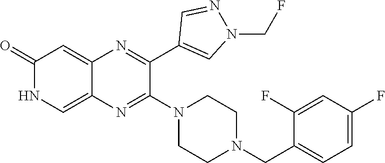 [SMILES: O=c1cc2nc(-c3cnn(CF)c3)c(N3CCN(Cc4ccc(F)cc4F)CC3)nc2c[nH]1] | 3-{4-[(2,4- difluorophenyl)methyl]- 1-piperazinyl}-2-[1- (fluoromethyl)-4-pyrazolyl]- 1,4,6-triaza- 7(6H)-naphthalenone | NA |
| 797 | 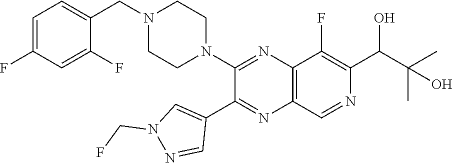 [SMILES: CC(C)(O)C(O)c1ncc2nc(-c3cnn(CF)c3)c(N3CCN(Cc4ccc(F)cc4F)CC3)nc2c1F] | 1-(2-{4-[(2,4- difluorophenyl)methyl]-1- piperazinyl}-8-fluoro-3- [1-(fluoromethyl)-4- pyrazolyl]-1,4,6-triaza-7- naphthyl)-2-methyl-1,2- propanediol | Racemate |
| 798 | 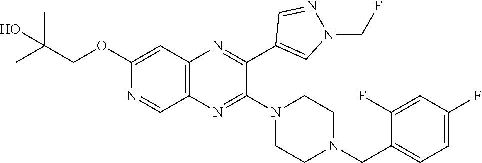 [SMILES: CC(C)(O)COc1cc2nc(-c3cnn(CF)c3)c(N3CCN(Cc4ccc(F)cc4F)CC3)nc2cn1] | 1-(3-{4-[(2,4- difluorophenyl)methyl]-1- piperazinyl}-2-[1-(fluoromethyl)- 4-pyrazolyl]-1,4,6-triaza-7- naphthyloxy)-2-methyl-2-propanol | NA |
| 799 | 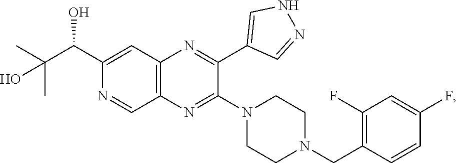 [SMILES: CC(C)(O)[C@H](O)c1cc2nc(-c3cn[nH]c3)c(N3CCN(Cc4ccc(F)cc4F)CC3)nc2cn1] or enantiomer | (R)-1-(3-{4-[(2,4- difluorophenyl)methyl]-1- piperazinyl}-2-(4-pyrazolyl)-1,4,6- triaza-7-naphthyl)-2-methyl-1,2- propanediol | (−) Isomer |
| 800 | 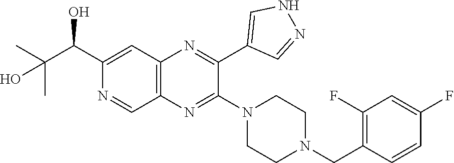 [SMILES: CC(C)(O)[C@@H](O)c1cc2nc(-c3cn[nH]c3)c(N3CCN(Cc4ccc(F)cc4F)CC3)nc2cn1] or enantiomer | (S)-1-(3-{4-[(2,4- difluorophenyl)methyl]-1- piperazinyl}-2-(4-pyrazolyl)-1,4,6- triaza-7-naphthyl)-2-methyl-1,2- propanediol | (+) Isomer |

^{[1]}NA means the concat optical rotation data for this compound is not available or not applicable. The concat optical rotation data are obtained through the procedure included in example 58.

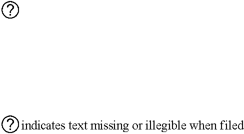

### NUMBERED EMBODIMENTS

1. A compound of Formula (I), or a pharmaceutically acceptable salt thereof:

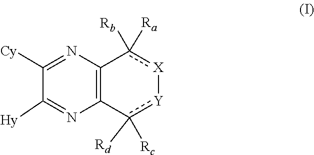

wherein
Rₐ, R_{b}, R_{c}, and R_{d} are each independently absent, H, alkyl, halogen, —CN, —CH₂NRₑR_{f}, —NRₑR_{f}, —NRₑC(═O)R_{f}, —NRₑC(═O)NR_{f}R_{g}, —NRₑC(═O)OR_{f}, —NRₑSO₂R_{f}, —OC(═O)NRₑR_{f}, —ORₑ, —SRₑ, —SORₑ, —SO₂Rₑ, —SO₂ORₑ, —SO₂NRₑR_{f}, —C(═O)Rₑ, —C(═O)ORₑ, —C(═O)NRₑR_{f}, (hetero)alkyl, (hetero)alkenyl, (hetero)alkynyl, spirocyclyl, spiroheterocyclyl, carbocyclyl, or heterocyclyl; Rₐ and R_{b} together with the atoms to which they are attached form ═NSO₂Rₑ, ═O, carbocyclyl, or heterocyclyl; R_{c} and R_{d} together with the atoms to which they are attached form ═NSO₂Rₑ, ═O, carbocyclyl, or heterocyclyl; or Rₐ and R_{c} together with the atoms to which they are attached form a carbocyclyl or heterocyclyl; Rₐ and Y together with the atoms to which they are attached form a carbocyclyl or heterocyclyl; R_{c} and X together with the atoms to which they are attached form a carbocyclyl or heterocyclyl;
each of X and Y is independently selected from the group consisting of O, N, NRₑ, N(C(Rₑ)₂C(═O)NHR_{f}), N(C(═O)Rₑ), N(C(═O)ORₑ), NC(═O)C(Rₑ)₃, S, S(═O), S(═O)₂, S(═O)(═NRₑ), S(═NRₑ)₂, C(CN), C(CN)Rₑ, C(C(═O)Rₑ), C(Rₕ), NS(O)₂NRₑRₕ, NS(O)NRₑRₕ, C(═NSO₂Rₑ), and C(Rₕ)₂;
each Rₑ, R_{f}, and R_{g} is independently H, —OH, —CN, —OCHF₂, ═O, alkoxy, alkyl, (hetero)alkyl, (hetero)alkenyl, (hetero)alkynyl, carbocyclyl, or heterocyclyl;
Hy is a carbon-linked 3-, 4-, 5- or 6-membered carbocyclic ring or a carbon-linked 3-, 4-, 5- or 6-membered heterocyclic ring comprising at least one N; and
Cy is -A-L-B;
wherein
A is a saturated 4- to 7-membered hetero- or carbocyclic ring, a saturated bicyclic or heterobicyclic ring, or
a saturated spirocyclic or spiroheterocyclic ring;
B is a 5- to 7-membered hetero- or carbocyclic ring; and
L is absent, C(Rₕ)₂, C(═O), C(═NSO₂Rₑ), NRₑ, O, S, N, S(═O), S(═O)₂, S(═O)(═NRₑ), or S(═NRₑ)₂, wherein when L is absent or NRₑ, B is aryl or heteroaryl,
wherein
each Rₕ is independently H, halogen, —CN, —OCHF₂, alkyl, alkoxy, —NRₑR_{f}, —NRₑC(═O)R_{f}, —NRₑC(═O)NR_{f}R_{g}, —NRₑC(═O)OR_{f}, —NRₑSO₂R_{f}, —OC(═O)NRₑR_{f}, —ORₑ, —SRₑ, —SORₑ, —SO₂Rₑ, —SO₂ORₑ, —SO₂NRₑR_{f}, —C(═O)Rₑ, —C(═O)ORₑ, —C(═O)NRₑR_{f}, (hetero)alkyl, (hetero)alkenyl, (hetero)alkynyl, carbocyclyl, or heterocyclyl; or two Rₕ attached to the same carbon atom together with the atoms to which they are attached form ═NSO₂Rₑ, ═O, carbocyclyl, or heterocyclyl.
1a. A compound of Formula (I), or a pharmaceutically acceptable salt thereof:

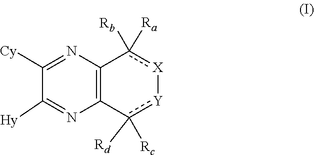

wherein
Rₐ, R_{b}, R_{c}, and R_{d} are each independently absent, H, alkyl, alkenyl, halogen, —CN, —CH₂NRₑR_{f}, —NRₑR_{f}, —NRₑC(═O)R_{f}, —NRₑC(═O)NR_{f}R_{g}, —NRₑC(═O)OR_{f}, —NRₑSO₂R_{f}, —OC(═O)NRₑR_{f}, —ORₑ, —SRₑ, —SORₑ, —SO₂Rₑ, —SO₂ORₑ, —SO₂NRₑR_{f}, —C(═O)Rₑ, —C(═O)ORₑ, —C(═O)C(CH₃)₂OH, —C(═O)NRₑR_{f}, spirocyclyl, spiroheterocyclyl, carbocyclyl, or heterocyclyl; Rₐ and R_{b} together with the atoms to which they are attached form ═NSO₂Rₑ, ═O, carbocyclyl, or heterocyclyl; R_{c} and R_{d} together with the atoms to which they are attached form ═NSO₂Rₑ, ═O, carbocyclyl, or heterocyclyl; or Rₐ and R_{c} together with the atoms to which they are attached form a carbocyclyl or heterocyclyl; Rₐ and Y together with the atoms to which they are attached form a carbocyclyl or heterocyclyl; R_{c} and X together with the atoms to which they are attached form a carbocyclyl or heterocyclyl;
each of X and Y is independently O, N, NRₑ, N(C(Rₑ)₂C(═O)NHR_{f}), N(C(═O)Rₑ), N(C(═O)ORₑ), NC(═O)C(Rₑ)₃, NC(═O)C(Rₑ)₂(Rₑ)₃, S, S(═O), S(═O)₂, S(═O)(═NRₑ), S(═NRₑ)₂, C(CN), C(CN)Rₑ, C(C(═O)Rₑ), C(Rₕ), NS(O)₂NRₑRₕ, NS(O)NRₑRₕ, C(═NSO₂Rₑ), or C(Rₕ)₂; provided that X and Y cannot both be C(═O), C(CN), C(CN)Rₑ, C(C(═O)Rₑ) or C(Rₕ);
each Rₑ, R_{f}, and R_{g} is independently H, halogen, —OH, —CN, —OCHF₂, —C(CH₃)₂OH, —CHF₂, —CF₃, —CH₂OH, CH(CH₃)CN, —C((CH₃)₂CN), —NH₂, —CH₂CH₂OCH₃, —CH₂CH(OH)CH₂OH, alkoxy, alkyl, carbocyclyl, or heterocyclyl,
each Rₕ is independently H, halogen, OH, —CN, alkyl, —C(Rₑ)₃, —C(Rₑ)₂C(Rₑ)₃, —CH₂C(Rₑ)₂C(Rₑ)₃, —OCH₂C(Rₑ)₃, —NHCH₂C(Rₑ)₃, —N(alkyl)CH₂C(Rₑ)₃, —O-heterocyclyl, alkoxy, haloalkoxy, —NRₑR_{f}, —NRₑC(═O)R_{f}, —NRₑC(═O)NR_{f}R_{g}, —CH₂NRₑC(═O)NR_{f}R_{g}, —NRₑC(═O)OR_{f}, —NRₑSO₂R_{f}, —OC(═O)NRₑR_{f}, —ORₑ, —SRₑ, —SORₑ, —SO₂Rₑ, —SO₂ORₑ, —SO₂NRₑR_{f}, —C(═O)Rₑ, —C(═O)ORₑ, —C(═O)NRₑR_{f}, carbocyclyl, aryl or heterocyclyl; or two Rₕ attached to the same carbon atom together with the atoms to which they are attached form ═NSO₂Rₑ, ═O, carbocyclyl, heteroaryl or heterocyclyl;
Hy is a carbon-linked 3-, 4-, 5- or 6-membered carbocyclyl; a carbon-linked 3-10-membered monocyclic heterocyclyl; or a carbon-linked 3-10-membered bicyclic heterocyclyl; and
Cy is -A-L-B or -A=L-B;
wherein
A is a nitrogen-containing heterocyclylene linked by a nitrogen atom to

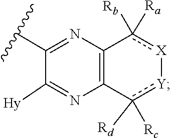

B is a 5- to 7-membered heterocyclyl, a 5- to 7-membered carbocyclyl, or —C(═O)C₁₋₆alkyl; and
L is absent, C(Rₕ), C(Rₕ)₂, OC(Rₕ)₂, C(═O), C(═NSO₂Rₑ), C(═O)N(Rₑ), NRₑ, O, S, N, S(═O), S(═O)₂, S(═O)(═NRₑ), or S(═NRₑ)₂, wherein when L is absent or NRₑ, B is aryl or heteroaryl.
2. A compound of Formula (I′), or a pharmaceutically acceptable salt thereof:

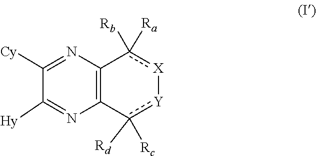

wherein
Rₐ, R_{b}, R_{c}, and R_{d} are each independently absent, H, alkyl, halogen, —CN, —CH₂NRₑR_{f}, —NRₑR_{f}, —NRₑC(═O)R_{f}, —NRₑC(═O)NR_{f}R_{g}, —NRₑC(═O)OR_{f}, —NRₑSO₂R_{f}, —OC(═O)NRₑR_{f}, —ORₑ, —SRₑ, —SORₑ, —SO₂Rₑ, —SO₂ORₑ, —SO₂NRₑR_{f}, —C(═O)Rₑ, —C(═O)ORₑ, —C(═O)NRₑR_{f}, (hetero)alkyl, (hetero)alkenyl, (hetero)alkynyl, spirocyclyl, spiroheterocyclyl, carbocyclyl, or heterocyclyl; Rₐ and R_{b} together with the atoms to which they are attached form ═NSO₂Rₑ, ═O, carbocyclyl, or heterocyclyl; R_{c} and R_{d} together with the atoms to which they are attached form ═NSO₂Rₑ, ═O, carbocyclyl, or heterocyclyl; or Rₐ and R_{c} together with the atoms to which they are attached form a carbocyclyl or heterocyclyl; Rₐ and Y together with the atoms to which they are attached form a carbocyclyl or heterocyclyl; R_{c} and X together with the atoms to which they are attached form a carbocyclyl or heterocyclyl;
each of X and Y is independently selected from the group consisting of O, N, NRₑ, N(C(Rₑ)₂C(═O)NHR_{f}), N(C(═O)Rₑ), N(C(═O)ORₑ), NC(═O)C(Rₑ)₃, S, S(═O), S(═O)₂, S(═O)(═NRₑ), S(═NRₑ)₂, NS(═O)₂Rₑ, C(CN), C(CN)Rₑ, C(C(═O)Rₑ), C(Rₕ), NS(O)₂NRₑRₕ, NS(O)NRₑRₕ, C(═NSO₂Rₑ), and C(Rₕ)₂, wherein at least one of X and Y is O, N, NRₑ, N(C(Rₑ)₂C(═O)NHR_{f}), N(C(═O)Rₑ), N(C(═O)ORₑ), NC(═O)C(Rₑ)₃, S, S(═O), S(═O)₂, S(═O)(═NRₑ), S(═NRₑ)₂, NS(═O)₂Rₑ, NS(O)₂NRₑRₕ, or NS(O)NRₑRₕ;
each Rₑ, R_{f}, and R_{g} is independently H, —OH, —CN, —OCHF₂, —CF₃, —CH₂OH, CH(CH₃)CN, —C((CH₃)₂CN), —NHCH₃, —N(CH₃)₂, —CH₂CH(OH)CH₂OH, ═O, alkoxy, alkyl, (hetero)alkyl, (hetero)alkenyl, (hetero)alkynyl, carbocyclyl, or heterocyclyl;
Hy is a carbon-linked 3-, 4-, 5- or 6-membered carbocyclic ring or a carbon-linked 3-, 4-, 5- or 6-membered heterocyclic ring comprising one or more S or O; and
Cy is -A-L-B;
wherein
A is a piperidine or piperazine ring;
B is a 5- to 7-membered hetero- or carbocyclic ring; and
L is absent, C(Rₕ)₂, C(═O), C(═NSO₂Rₑ), NRₑ, O, S, N, S(═O), S(═O)₂, S(═O)(═NRₑ), or S(═NRₑ)₂, wherein when L is absent or NRₑ, B is aryl or heteroaryl,
wherein
each Rₕ is independently H, halogen, —CN, —OCHF₂, alkyl, alkoxy, —CH₂OH, —CH(OH)C(Rₑ)₃, —CH(OH)CRₑ, —NRₑR_{f}, —NR_{c}C(═O)R_{f}, —NRₑC(═O)NR_{f}R_{g}, —NRₑC(═O)OR_{f}, —NRₑSO₂R_{f}, —OC(═O)NRₑR_{f}, —ORₑ, —SRₑ, —SORₑ, —SO₂Rₑ, —SO₂ORₑ, —SO₂NRₑR_{f}, —C(═O)Rₑ, —C(═O)ORₑ, —C(═O)NRₑR_{f}, (hetero)alkyl, (hetero)alkenyl, (hetero)alkynyl, carbocyclyl, or heterocyclyl; or two Rₕ attached to the same carbon atom together with the atoms to which they are attached form ═NSO₂Rₑ, ═O, carbocyclyl, or heterocyclyl.
3. The compound of embodiment 1, 1a or 2, which is a compound of Formula (Ia), or a pharmaceutically acceptable salt thereof:

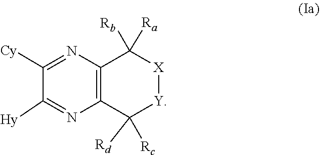

4. The compound of embodiment 1, 1a or 2, which is a compound of Formula (Ib), or a pharmaceutically acceptable salt thereof:

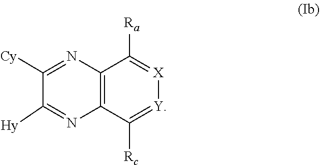

5. The compound of embodiment 1, 1a or 2, which is a compound of Formula (Ic), or a pharmaceutically acceptable salt thereof:

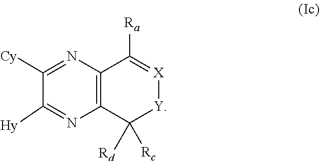

6. The compound of embodiment 1, 1a or 2, which is a compound of Formula (Id), or a pharmaceutically acceptable salt thereof:

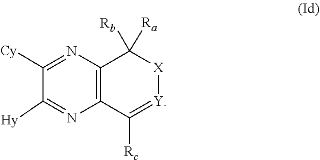

7. The compound of any one of embodiments 1a, 1-3, wherein Rₐ, R_{b}, R_{c}, and R_{d} are H.
8. The compound of any one of embodiments 1, a, 2, and 4, wherein Rₐ and R_{c} are H.
9. The compound of any one of embodiments 1, a, 2, and 5, wherein Rₐ, R_{c}, and R_{d} are H.
10. The compound of any one of embodiments 1, 1a, 2 and 6, wherein Rₐ, R_{b}, and R_{c} are H.
11. The compound of any one of embodiments 1a, 1-3, and 7, wherein one of X and Y is N(C(═O)Rₑ), and Rₑ is alkyl, (hetero)alkyl, (hetero)alkenyl, (hetero)alkynyl, carbocyclyl, or heterocyclyl; and
   the other one of X and Y is CH₂.
12. The compound of embodiment 1a, 11, wherein X is N(C(═O)Rₑ).
13. The compound of embodiment 1a, 11, wherein Y is N(C(═O)Rₑ).
14. The compound of any one of embodiments 1a, 11 to 13, wherein Rₑ is alkyl.
15. The compound of embodiment 1a, 14, wherein Rₑ is CH₃.
16. The compound of embodiment 1, 1a, 2, 4 or 8, wherein one of X and Y is CH or N and the other of X and Y is C(CN), C(Rₑ), or C(C(═O)Rₑ), and Rₑ is alkyl, (hetero)alkyl, (hetero)alkenyl, (hetero)alkynyl, carbocyclyl, or heterocyclyl.
17. The compound of embodiment 16, wherein X is CH or N and Y is C(CN), C(Rₑ), or C(C(═O)Rₑ);
18. The compound of embodiment 16, wherein Y is CH or N and X is C(CN), C(Rₑ), or C(C(═O)Rₑ);
19. The compound of embodiment 1, 1a, 2, 4 or 8, wherein one of X and Y is CH and the other one of X and Y is N.
20. The compound of embodiment 19, wherein X is CH and Y is N.
21. The compound of embodiment 19, wherein Y is CH and X is N.
22. The compound of any one of embodiments 16 to 18, wherein Rₑ is alkyl.
23. The compound of embodiment 22, wherein Rₑ is CH₃ or CH₂(CH₃)₂.
24. The compound of any one of embodiments 1, 1a, 2, and 5, wherein one of X and Y is N(C(═O)Rₑ), and Rₑ is alkyl, (hetero)alkyl, (hetero)alkenyl, (hetero)alkynyl, carbocyclyl, or heterocyclyl; and
   the other one of X and Y is CH₂.
25. The compound of embodiment 24, wherein X is N(C(═O)Rₑ).
26. The compound of embodiment 24, wherein Y is N(C(═O)Rₑ).
27. The compound of any one of embodiments 24 to 26, wherein Rₑ is alkyl.
28. The compound of embodiment 27, wherein Rₑ is CH₃.
29. The compound of any one of embodiments 1, 1a, 2, 6 or 10, wherein one of X and Y is N(C(═O)Rₑ), and Rₑ is alkyl, (hetero)alkyl, (hetero)alkenyl, (hetero)alkynyl, carbocyclyl, or heterocyclyl; and
   the other one of X and Y is CH₂.
30. The compound of embodiment 29, wherein X is N(C(═O)Rₑ).
31. The compound of embodiment 29, wherein Y is N(C(═O)Rₑ).
32. The compound of any one of embodiments 29 to 31, wherein Rₑ is alkyl.
33. The compound of embodiment 32, wherein Rₑ is CH₃.
34. The compound of any one of embodiments 1 to 33, wherein Hy is

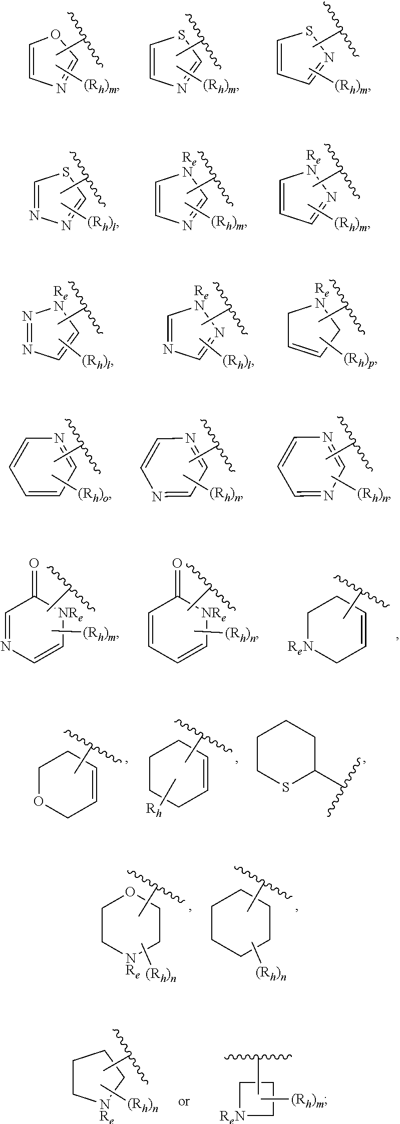

wherein l is 0 or 1; m is 0, 1, or 2; n is 0, 1, 2, or 3; o is 0, 1, 2, 3, or 4; and p is 0, 1, 2, 3, 4, or 5.
35. The compound of embodiment 34, wherein Hy is

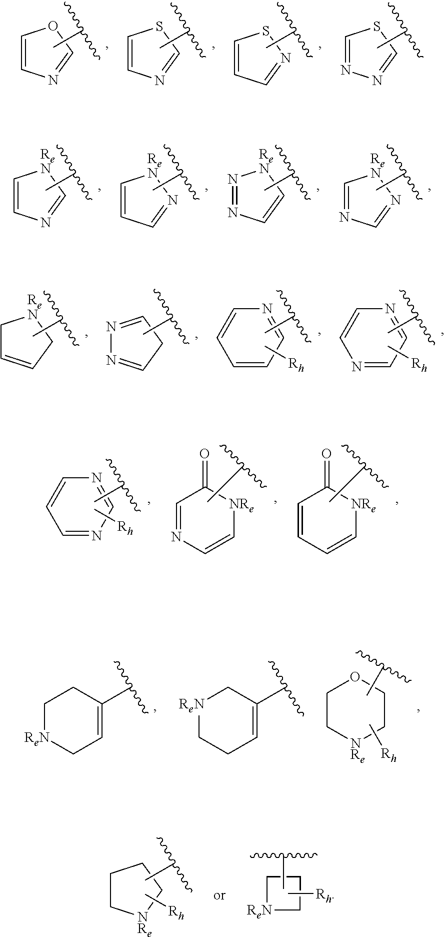

36. The compound of embodiment 35, wherein Hy is

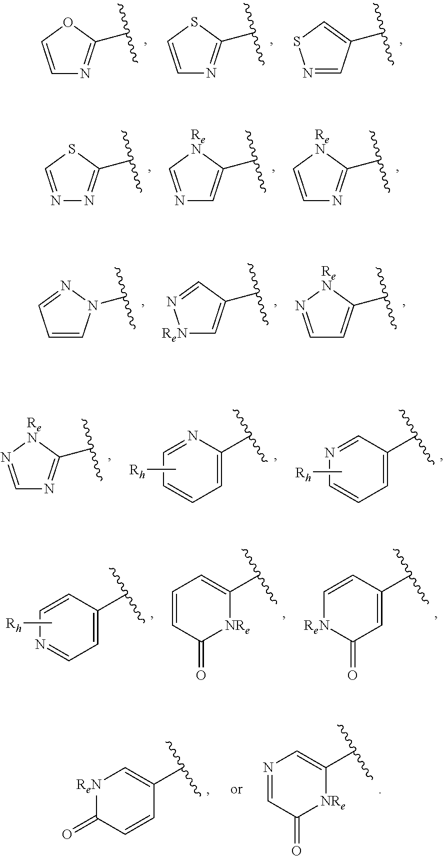

37. The compound of embodiment 36, wherein Hy is

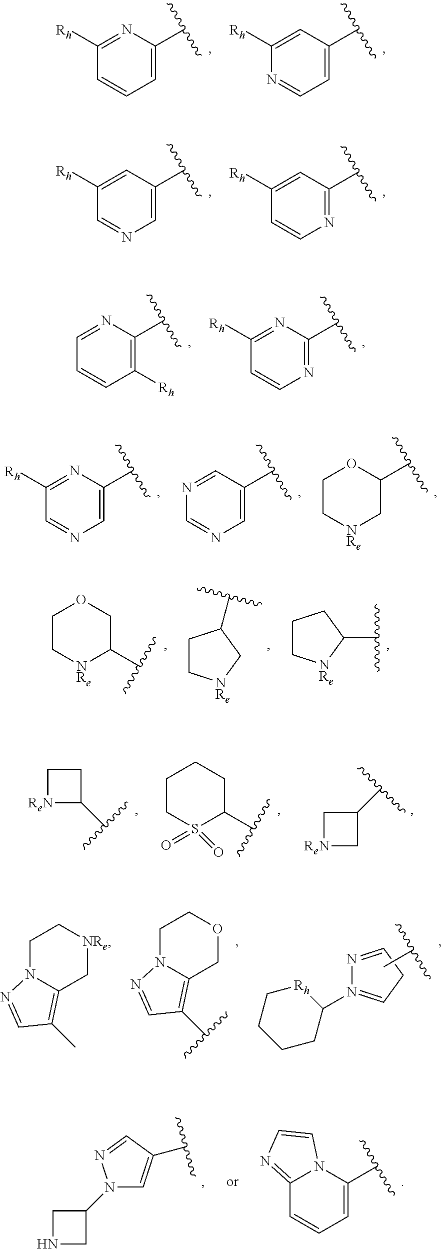

38. The compound of any one of embodiments 34 to 37, wherein Rₑ is H.
39. The compound of any one of embodiments 34 to 37, wherein Rₑ is OCH₃.
40. The compound of any one of embodiments 34 to 37, wherein Rₑ is CH₃.
41. The compound of any one of embodiments 34 to 37, wherein Rₑ is —C(═O)CH₃.
42. The compound of any one of embodiments 34 to 37, wherein Rₕ is heterocyclic.
43. The compound of any one of embodiments 34 to 37, wherein Rₕ is OCH₃.
44. The compound of any one of embodiments 1a, 1 to 43, wherein A is

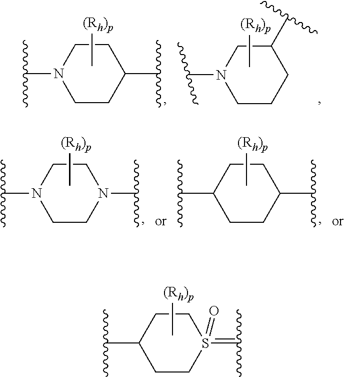

and p is 0, 1, 2, 3, or 4.
45. The compound of embodiment 44, wherein p is 0.
46. The compound of embodiment 44, wherein p is 1.
47. The compound of any one of embodiments 1 to 43, wherein A is

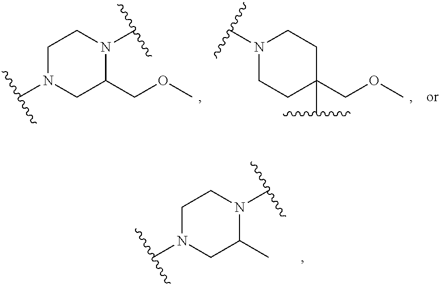

48. The compound of any one of embodiments 1a, 1 to 43, wherein A is

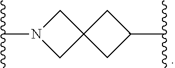 [SMILES: *C1CC2(C1)CN(*)C2]

49. The compound of any one of embodiments 1a, 1 to 43, wherein A is

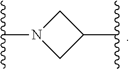 [SMILES: *C1CN(*)C1]

50. The compound of embodiments 1a, 1 to 43, wherein A is

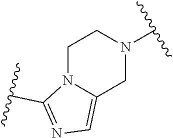 [SMILES: *c1ncc2n1CCN(*)C2]

51. The compound of any one of embodiments 1a, 1 to 50, wherein L is 0, CH₂, C(═O), NRₑ or N.
52. The compound of embodiment 51, wherein Rₑ is CH₃.
53. The compound of any one of embodiments 1a, 1 to 52, wherein B is

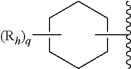

wherein q is 0, 1, 2, 3, 4, or 5.
54. The compound of any one of embodiments 1a, 1 to 52, wherein B is

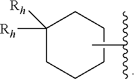

55. The compound of any one of embodiments 1a, 1 to 52, wherein B is

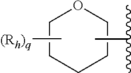

56. The compound of any one of embodiments 1a, 1 to 52, wherein B is

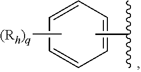

wherein q is 0, 1, 2, 3, 4, or 5.
57. The compound of embodiment 56, wherein q is 2.
58. The compound of embodiment 53, wherein B is

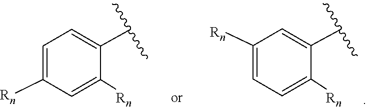

59. The compound of embodiment 58, wherein each Rₕ is independently halogen.
60. The compound of embodiment 58, wherein each Rₕ is independently F or Cl.
61. The compound of embodiment 58, wherein B is

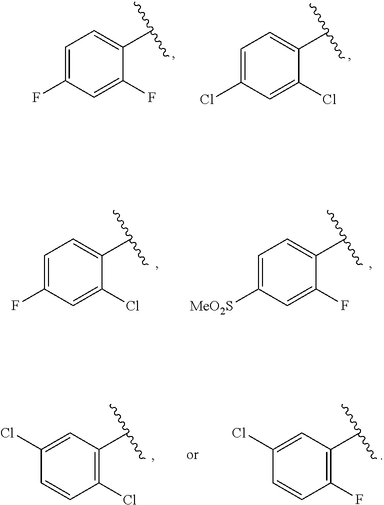 [SMILES: *c1ccc(F)cc1F ; *c1ccc(Cl)cc1Cl ; *c1ccc(F)cc1Cl ; *c1ccc(S(C)(=O)=O)cc1F ; *c1cc(Cl)ccc1Cl ; *c1cc(Cl)ccc1F]

62. The compound of any one of embodiments 1a, 1 to 43, wherein -A-L-B is

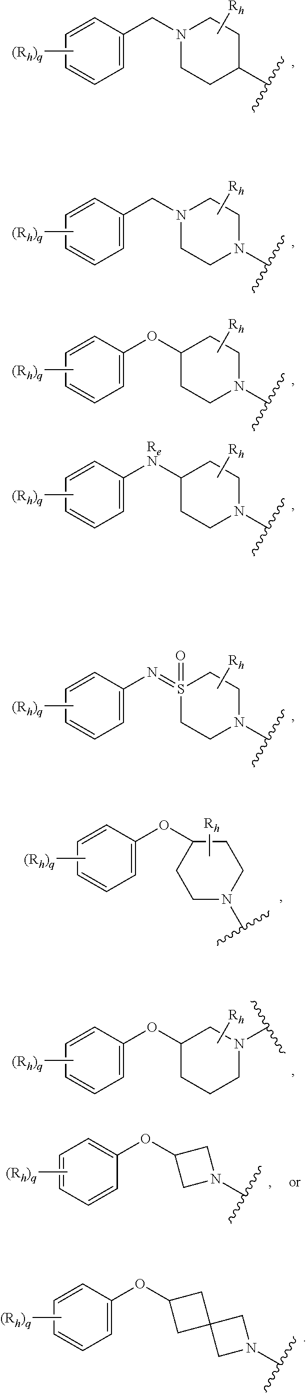

63. The compound of embodiment 62, wherein q is 2.
64. The compound of embodiment 62 or 63 wherein -A-L-B is

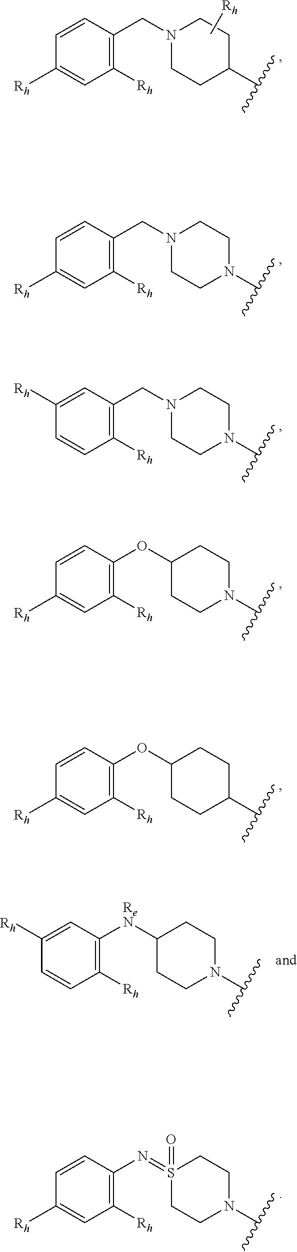

65. The compound of any one of embodiments 62 to 64, wherein -A-L-B is

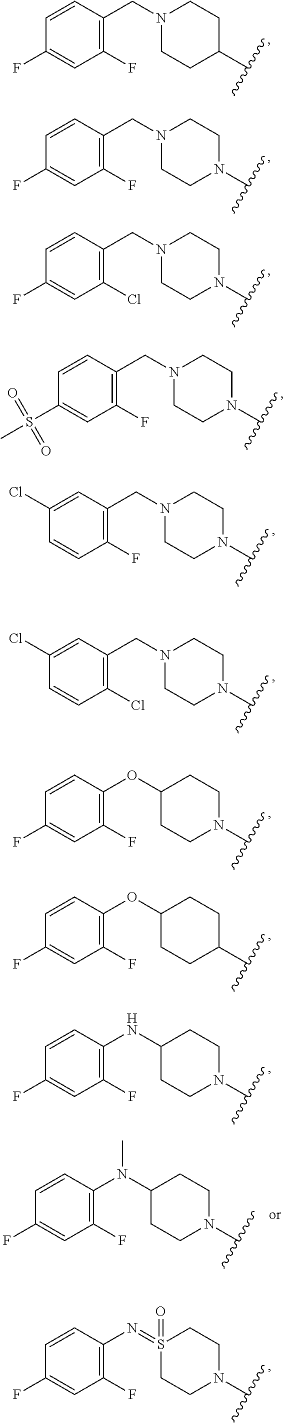

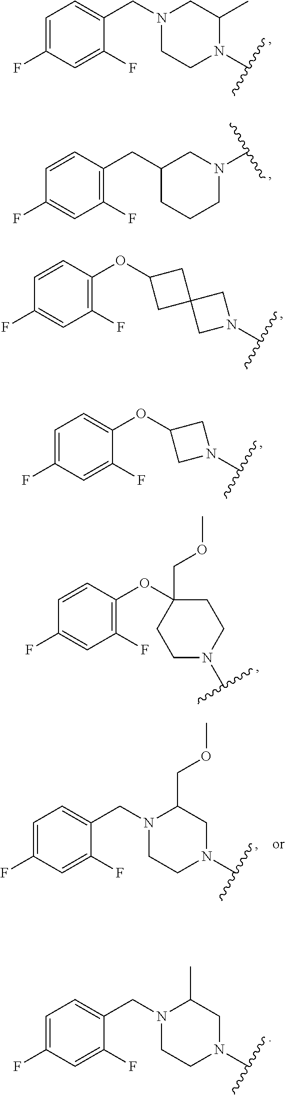

66. A compound selected from the group consisting of

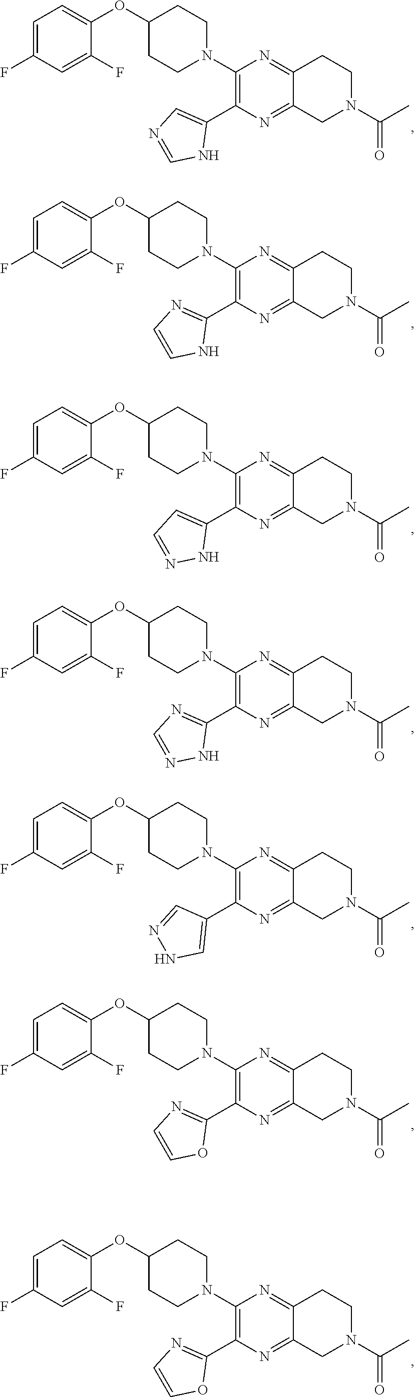

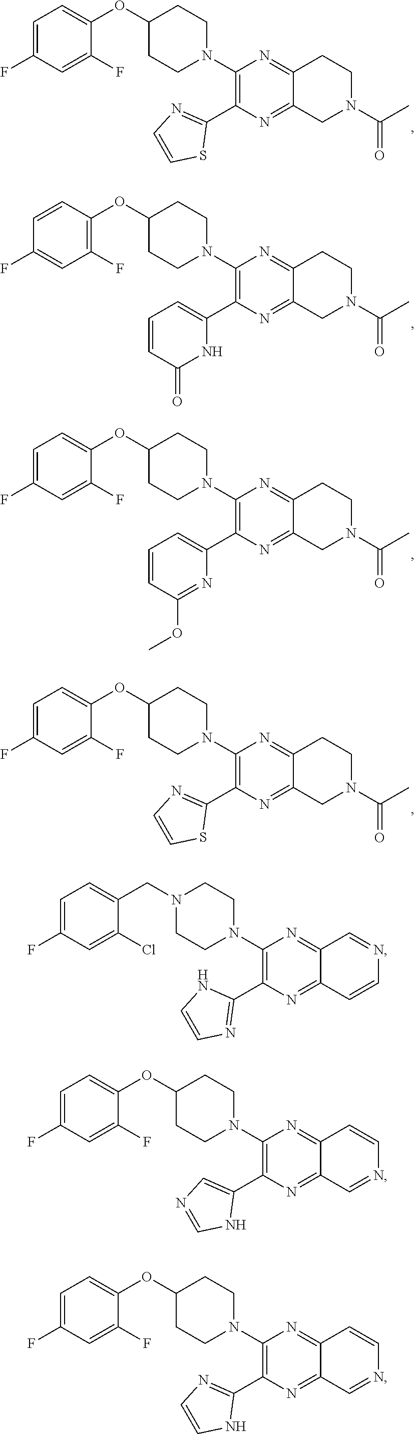

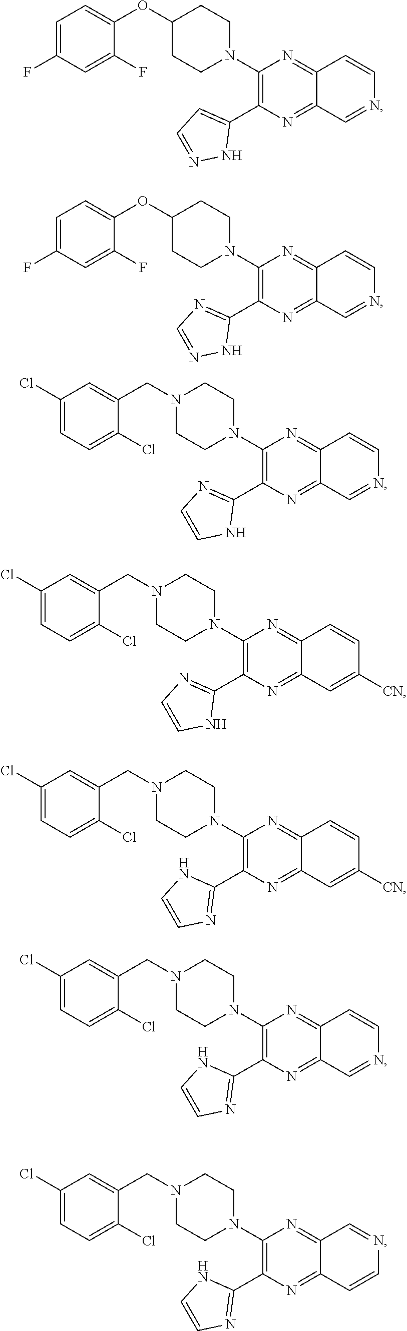

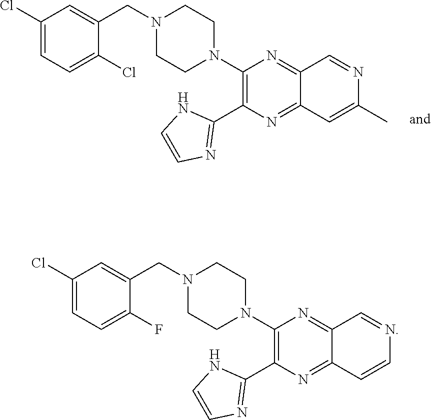 [SMILES: Cc1cc2nc(-c3ncc[nH]3)c(N3CCN(Cc4cc(Cl)ccc4Cl)CC3)nc2cn1 ; Fc1ccc(Cl)cc1CN1CCN(c2nc3cnccc3nc2-c2ncc[nH]2)CC1]

67. A compound of Formula (Ib), or a pharmaceutically acceptable salt thereof:

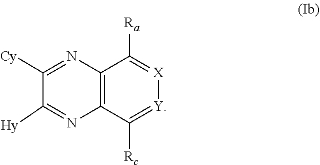

wherein
Rₐ and R_{c}, are each independently absent, H, alkyl, halogen, —CN, —CH₂NRₑR_{f}, —NRₑR_{f}, —NRₑC(═O)R_{f}, —NRₑC(═O)NR_{f}R_{g}, —NRₑC(═O)OR_{f}, —NRₑSO₂R_{f}, —OC(═O)NRₑR_{f}, —ORₑ, —SRₑ, —SORₑ, —SO₂Rₑ, —SO₂ORₑ, —SO₂NRₑR_{f}, —C(═O)Rₑ, —C(═O)ORₑ, —C(═O)NRₑR_{f}, (hetero)alkyl, (hetero)alkenyl, (hetero)alkynyl, spirocyclyl, spiroheterocyclyl, carbocyclyl, or heterocyclyl; or Rₐ and R_{c} together with the atoms to which they are attached form a carbocyclyl or heterocyclyl; Rₐ and Y together with the atoms to which they are attached form a carbocyclyl or heterocyclyl; R_{c} and X together with the atoms to which they are attached form a carbocyclyl or heterocyclyl;
each of X and Y is independently selected from the group consisting of N, C(CN) or C(Rₕ);
each Rₑ, R_{f}, and R_{g} is independently H, —OH, —CN, —OCHF₂, ═O, —CHF₂, alkoxy, alkyl, (hetero)alkyl, (hetero)alkenyl, (hetero)alkynyl, carbocyclyl, or heterocyclyl;
Hy is a carbon-linked 3-, 4-, 5- or 6-membered carbocyclic ring or a carbon-linked 3-, 4-, 5- or 6-membered heterocyclic ring comprising one or more N, S, or O; and
Cy is -A-L-B;
wherein
A is a saturated 4- to 7-membered hetero- or carbocyclic ring, a saturated bicyclic or heterobicyclic ring, or a saturated spirocyclic or spiroheterocyclic ring; and
B is a 5- to 7-membered hetero- or carbocyclic ring; and
L is absent, C(Rₕ)₂, C(═O), C(═NSO₂Rₑ), NRₑ, O, S, N, S(═O), S(═O)₂, S(═O)(═NRₑ), or S(═NRₑ)₂, wherein when L is absent or NRₑ, B is aryl or heteroaryl,
wherein
each Rₕ is independently H, halogen, —CN, —OCHF₂, alkyl, alkoxy, —CH₂OH, —CH(OH)C(Rₑ)₃, —CH(OH)Rₑ, —NRₑR_{f}, —NRₑC(═O)R_{f}, —NRₑC(═O)NR_{f}R_{g}, —NRₑC(═O)OR_{f}, —NRₑSO₂R_{f}, —OC(═O)NRₑR_{f}, —ORₑ, —SRₑ, —SORₑ, —SO₂Rₑ, —SO₂ORₑ, —SO₂NRₑR_{f}, —C(═O)Rₑ, —C(═O)ORₑ, —C(═O)NRₑR_{f}, (hetero)alkyl, (hetero)alkenyl, (hetero)alkynyl, carbocyclyl, or heterocyclyl; or two Rₕ attached to the same carbon atom together with the atoms to which they are attached form ═NSO₂Rₑ, ═O, carbocyclyl, or heterocyclyl.
68. The compound of embodiment 67, wherein Rₐ and R_{c} are H.
69. The compound of embodiment 68, wherein one of X and Y is CH or N and the other of X and Y is C(Rₕ), and Rₕ is alkyl, (hetero)alkyl, or CH(OH)CRₑR_{f}R_{g}.
70. The compound of embodiment 69, wherein Rₕ is CH(OH)C(CH₃)₂OH.
71. The compound of any one of embodiments 67-70, wherein Hy is

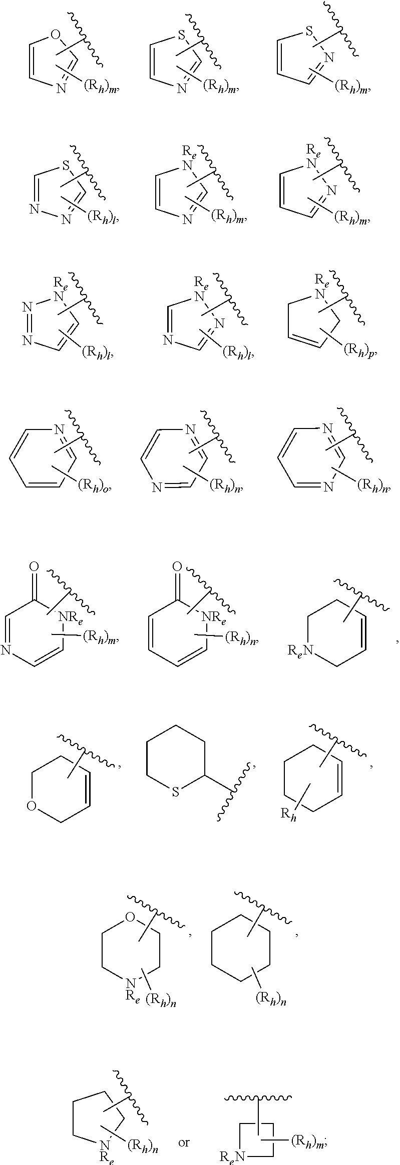

wherein l is 0 or 1; m is 0, 1, or 2; n is 0, 1, 2, or 3; o is 0, 1, 2, 3, or 4; and p is 0, 1, 2, 3, 4, or 5.
72. The compound of embodiment 71, wherein Hy is

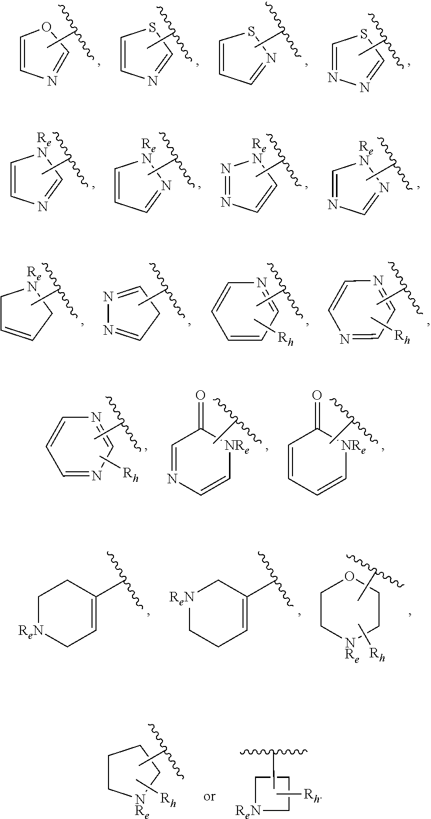

73. The compound of embodiment 72, wherein Hy is

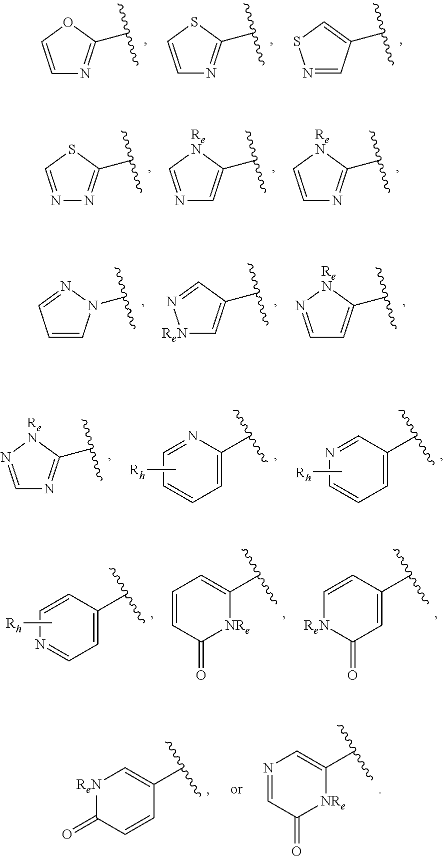

74. The compound of embodiment 73, wherein Hy is

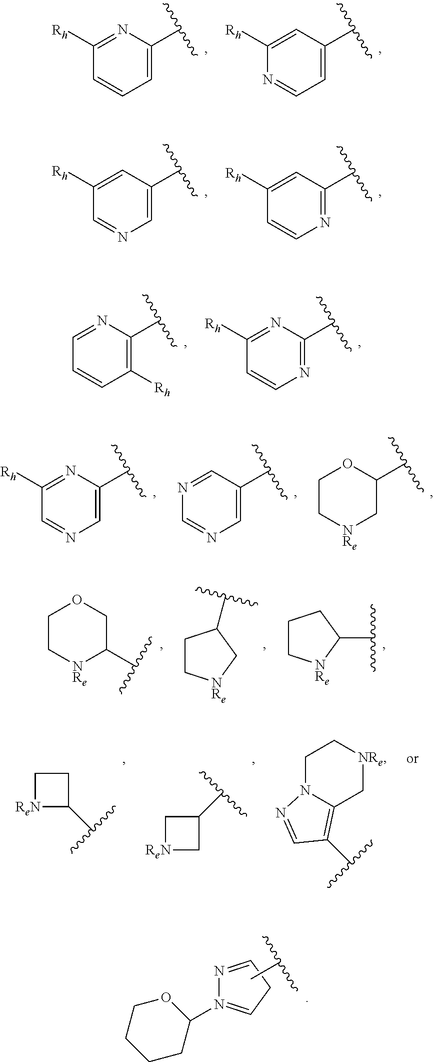

75. The compound of any one of embodiments 72 to 74, wherein Rₑ is H.
76. The compound of any one of embodiments 72 to 74, wherein Rₑ is OCH₃.
77. The compound of any one of embodiments 72 to 74, wherein Rₑ is CH₃.
78. The compound of any one of embodiments 72 to 74, wherein Rₑ is —C(═O)CH₃.
79. The compound of any one of embodiments 72 to 74, wherein R is heterocyclic.
80. The compound of any one of embodiments 72 to 74, wherein Rₕ is OCH₃.
81. The compound of any one of embodiments 72 to 74, wherein Rₕ is O or NRₑ.
82. The compound of an one of embodiments 1a, 1 to 81, wherein A is

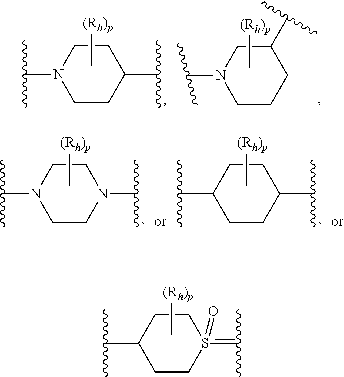

and p is 0, 1, 2, 3, or 4.
83. The compound of embodiment 28, wherein p is 0.
84. The compound of embodiment 28, wherein p is 1.
85. The compound of any one of embodiments 1a, 1 to 81, wherein A is

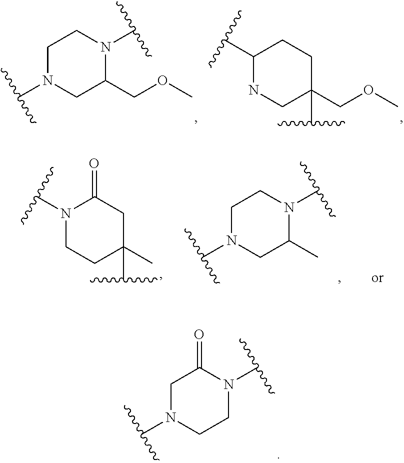

86. The compound of any one of embodiments 1a, 1 to 81, wherein A is

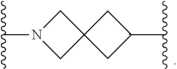 [SMILES: *C1CC2(C1)CN(*)C2]

87. The compound of any one of embodiments 1a, 1 to 81, wherein A is

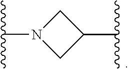 [SMILES: *C1CN(*)C1]

88. The compound of embodiments 1 to 81, wherein A is

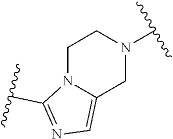 [SMILES: *c1ncc2n1CCN(*)C2]

89. The compound of any one of embodiments 1a, 1 to 88, wherein L is 0, CH₂, C(═O), NRₑ or N.
90. The compound of embodiment 89, wherein Rₑ is CH₃.
91. The compound of any one of embodiments 1a, 1 to 90, wherein B is

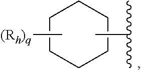

wherein q is 0, 1, 2, 3, 4, or 5.
92. The compound of any one of embodiments 1a, 1 to 90, wherein B is

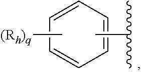

wherein q is 0, 1, 2, 3, 4, or 5.
93. The compound of embodiment 92 or 93, wherein q is 2.
94. The compound of embodiment 94, wherein B is

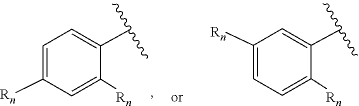

95. The compound of any one of embodiments 1a, 1 to 92, wherein B is

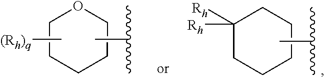

96. The compound of any one of embodiments 1a or 1 to 92, wherein B is

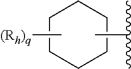

97. The compound of embodiment 96, wherein each Rₕ is independently halogen.
98. The compound of embodiment 97, wherein each Rₕ is independently F or Cl.
99. The compound of embodiment 98, wherein B is

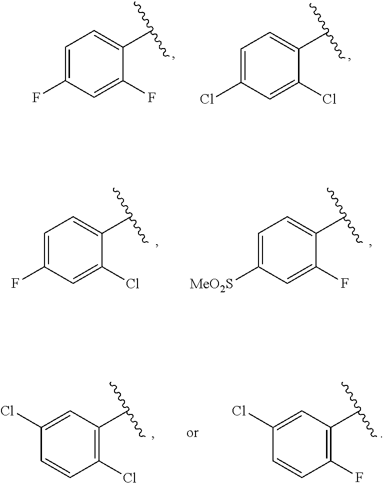 [SMILES: *c1ccc(F)cc1F ; *c1ccc(Cl)cc1Cl ; *c1ccc(F)cc1Cl ; *c1ccc(S(C)(=O)=O)cc1F ; *c1cc(Cl)ccc1Cl ; *c1cc(Cl)ccc1F]

100. The compound of any one of embodiments 1a, 1 to 81, wherein -A-L-B is

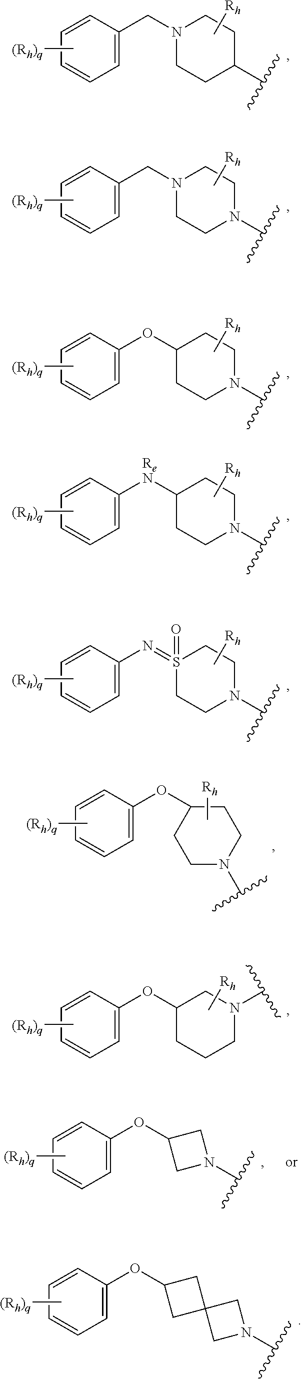

101. The compound of embodiment 100, wherein q is 2.
102. The compound of embodiment 100 or 101 wherein -A-L-B is

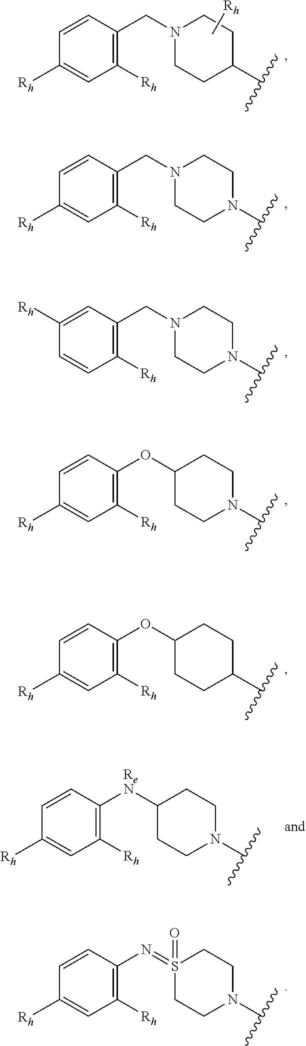

103. The compound of anyone of embodiments 100 to 102, wherein -A-L-B is

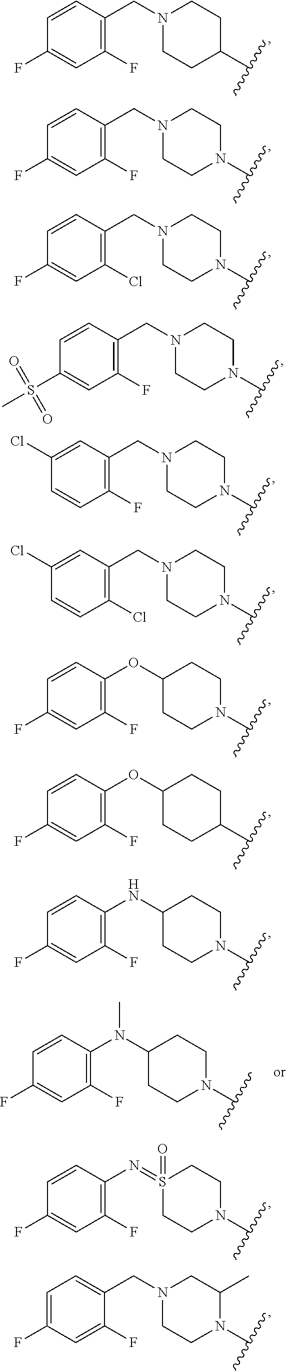

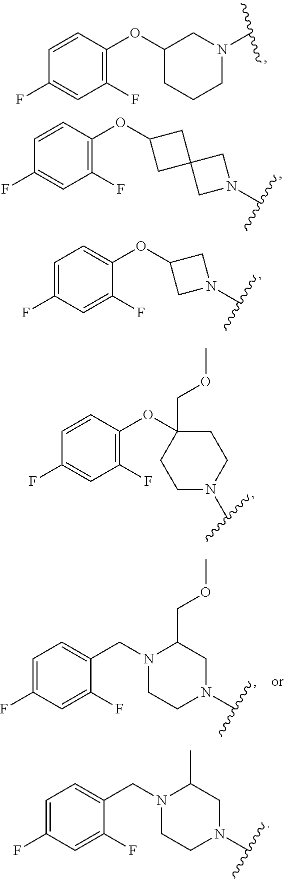

104. A compound selected from the group consisting of,

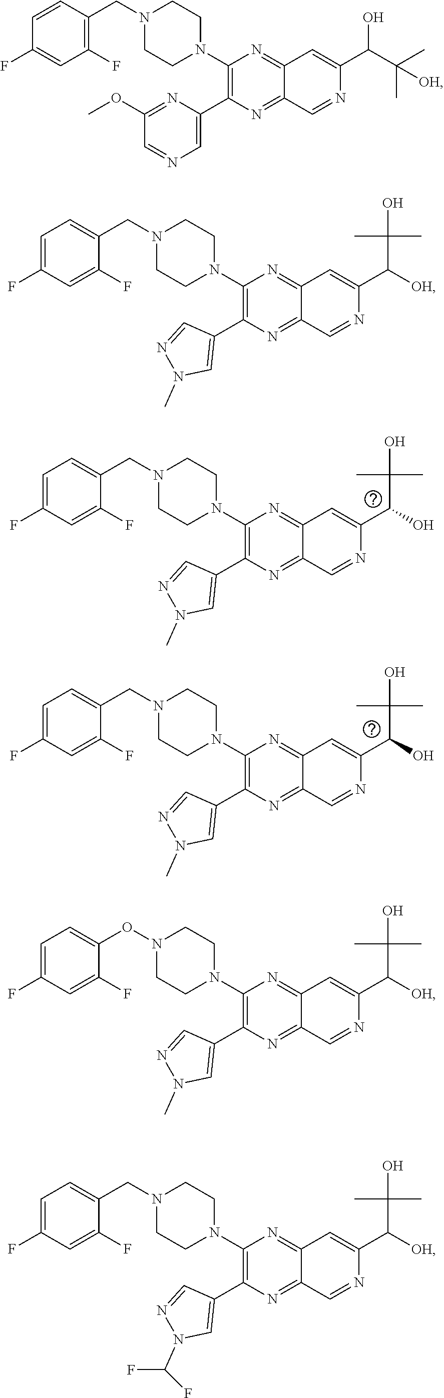 [SMILES: COc1cncc(-c2nc3cnc(C(O)C(C)(C)O)cc3nc2N2CCN(Cc3ccc(F)cc3F)CC2)n1 ; Cn1cc(-c2nc3cnc(C(O)C(C)(C)O)cc3nc2N2CCN(Cc3ccc(F)cc3F)CC2)cn1 ; Cn1cc(-c2nc3cnc([C@@H](O)C(C)(C)O)cc3nc2N2CCN(Cc3ccc(F)cc3F)CC2)cn1 ; Cn1cc(-c2nc3cnc([C@H](O)C(C)(C)O)cc3nc2N2CCN(Cc3ccc(F)cc3F)CC2)cn1 ; Cn1cc(-c2nc3cnc(C(O)C(C)(C)O)cc3nc2N2CCN(Oc3ccc(F)cc3F)CC2)cn1 ; CC(C)(O)C(O)c1cc2nc(N3CCN(Cc4ccc(F)cc4F)CC3)c(-c3cnn(C(F)F)c3)nc2cn1]

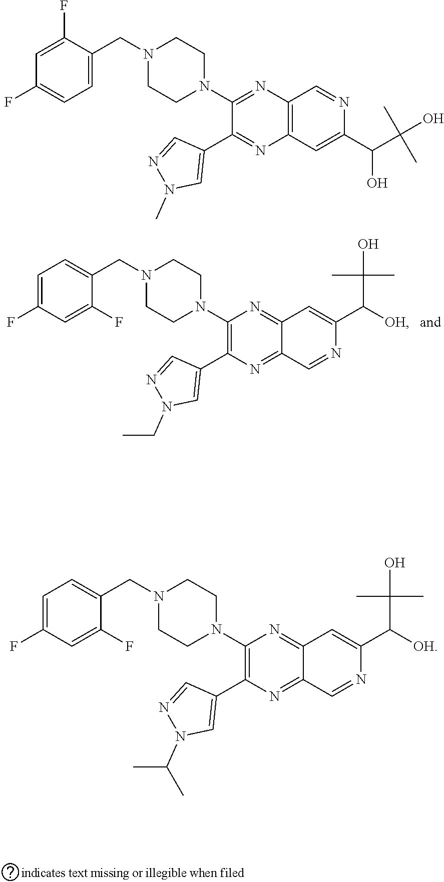

105. A compound of Formula (Ia), or a pharmaceutically acceptable salt thereof:

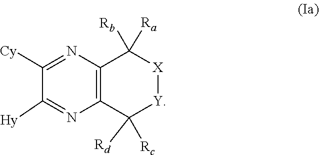

wherein
R_{b} and R_{d}, are each independently H;
Rₐ and R_{c}, are each independently absent, H, alkyl, halogen, —CN, —CH₂NRₑR_{f}, —NRₑR_{f}, —NRₑC(═O)R_{f}, —NRₑC(═O)NR_{f}R_{g}, —NRₑC(═O)OR_{f}, —NRₑSO₂R_{f}, —OC(═O)NRₑR_{f}, —ORₑ, —SRₑ, —SORₑ, —SO₂Rₑ, —SO₂ORₑ, —SO₂NRₑR_{f}, —C(═O)Rₑ, —C(═O)ORₑ, —C(═O)NRₑR_{f}, (hetero)alkyl, (hetero)alkenyl, (hetero)alkynyl, spirocyclyl, spiroheterocyclyl, carbocyclyl, or heterocyclyl; or Rₐ and R_{c} together with the atoms to which they are attached form a carbocyclyl or heterocyclyl; Rₐ and Y together with the atoms to which they are attached form a carbocyclyl or heterocyclyl; R_{c} and X together with the atoms to which they are attached form a carbocyclyl or heterocyclyl;
each of X and Y is independently selected from the group consisting of O, NRₑ, N(C(Rₑ)₂C(═O)NHR_{f}), N(C(═O)Rₑ), N(C(═O)ORₑ), NC(═O)C(Rₑ)₃, S, S(═O)₂, S(═O)(═NRₑ), S(═NRₑ)₂, NS(═O)₂Rₑ, C(CN)Rₑ, NS(O)₂NRₑRₕ, NS(O)NRₑRₕ, C(═NSO₂Rₑ), and C(Rₕ)₂;
each Rₑ, R_{f}, and R_{g} is independently H, —OH, —CN, —OCHF₂, —CHF₂, —CF₃, —CH₂OH, CH(CH₃)CN, —C((CH₃)₂CN), —NHCH₃, —N(CH₃)₂, —CH₂CH(OH)CH₂OH, alkoxy, alkyl, (hetero)alkyl, (hetero)alkenyl, (hetero)alkynyl, carbocyclyl, or heterocyclyl;
Hy is a carbon-linked 3-, 4-, 5- or 6-membered carbocyclic ring or a carbon-linked 3-, 4-, 5- or 6-membered heterocyclic ring comprising one or more N, S or O; and
Cy is -A-L-B;
wherein
A is a saturated 4- to 7-membered hetero- or carbocyclic ring, a saturated bicyclic or heterobicyclic ring, or
a saturated spirocyclic or spiroheterocyclic ring; and
B is a 5- to 7-membered hetero- or carbocyclic ring; and
L is absent, C(Rₕ)₂, C(═O), C(═NSO₂Rₑ), NRₑ, O, S, N, S(═O), S(═O)₂, S(═O)(═NRₑ), or S(═NRₑ)₂, wherein when L is absent or NRₑ, B is aryl or heteroaryl,
wherein
each Rₕ is independently H, halogen, —CN, —OCHF₂, alkyl, alkoxy, —CH₂OH, —CH(OH)C(Rₑ)₃, —CH(OH)Rₑ, —NRₑR_{f}, —NRₑC(═O)R_{f}, —NRₑC(═O)NR_{f}R_{g}, —NRₑC(═O)OR_{f}, —NRₑSO₂R_{f}, —OC(═O)NRₑR_{f}, —ORₑ, —SRₑ, —SORₑ, —SO₂Rₑ, —SO₂ORₑ, —SO₂NRₑR_{f}, —C(═O)Rₑ, —C(═O)ORₑ, —C(═O)NRₑR_{f}, (hetero)alkyl, (hetero)alkenyl, (hetero)alkynyl, carbocyclyl, or heterocyclyl; or two Rₕ attached to the same carbon atom together with the atoms to which they are attached form ═NSO₂Rₑ, ═O, carbocyclyl, or heterocyclyl.
106. The compound of embodiment 105, wherein Rₐ and R_{c} are H.
107. The compound of embodiment 106, wherein one of X and Y is CH or N and the other of X and Y is C(Rₕ), and Rₕ is alkyl, (hetero)alkyl, or CH(OH)C(Rₑ)₃.
108. The compound of any one of embodiments 105-107, wherein Hy is

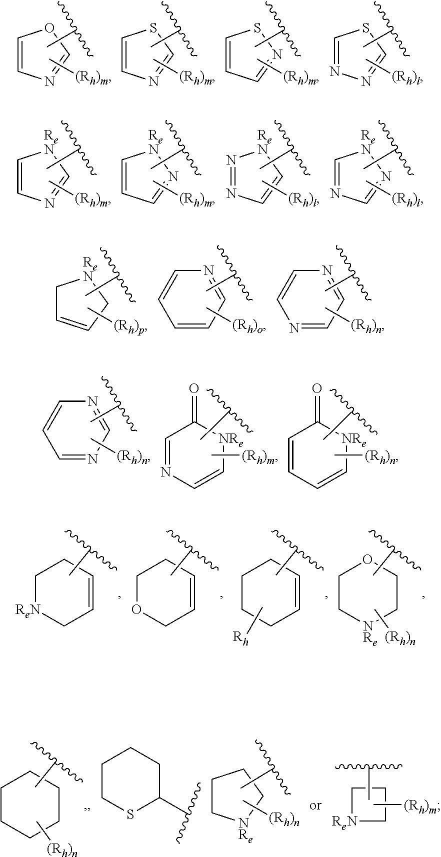

wherein l is 0 or 1; m is 0, 1, or 2; n is 0, 1, 2, or 3; o is 0, 1, 2, 3, or 4; and p is 0, 1, 2, 3, 4, or 5.
109. The compound of embodiment 108, wherein Hy is

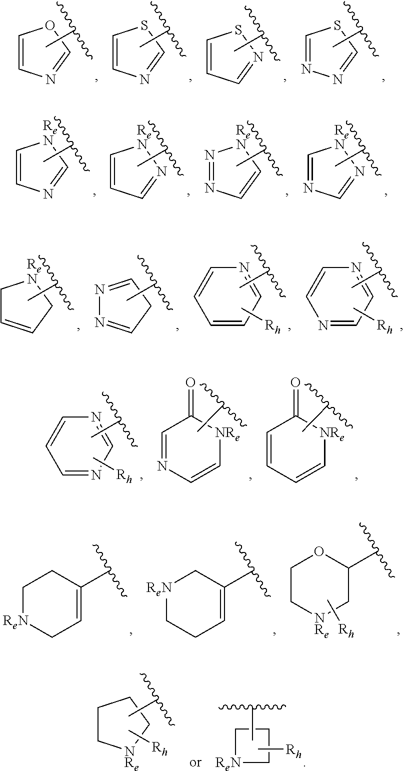

110. The compound of embodiment 109, wherein Hy is

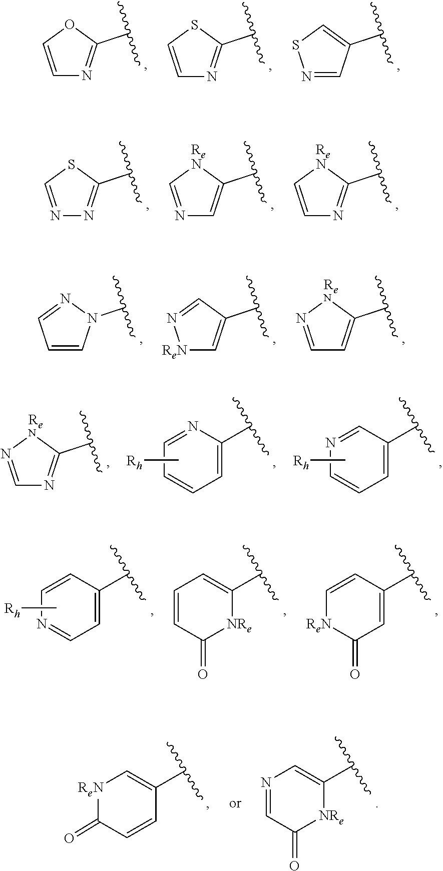

111. The compound of embodiment 110, wherein Hy is

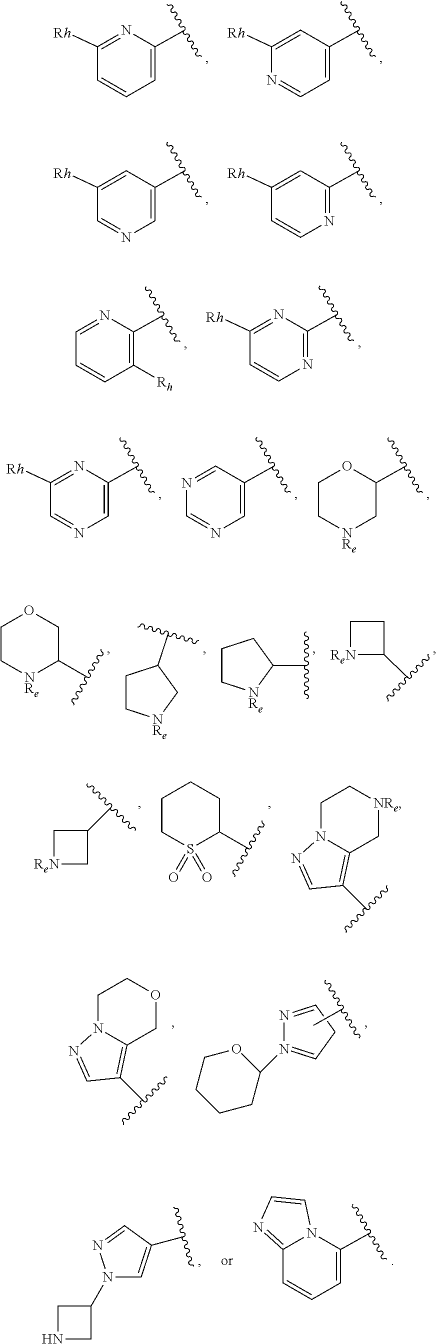

112. The compound of any one of embodiments 108 to 111, wherein Rₑ is H.
113. The compound of any one of embodiments 108 to 111, wherein Rₑ is OCH₃.
114. The compound of any one of embodiments 108 to 111, wherein Rₑ is CH₃.
115. The compound of any one of embodiments 108 to 111, wherein Rₑ is —C(═O)CH₃.
116. The compound of any one of embodiments 108 to 111, wherein Rₑ is heterocyclic.
117. The compound of any one of embodiments 108 to 111, wherein Rₕ is OCH₃.
118. The compound of any one of embodiments 108 to 111, wherein Rₕ is O or NRₑ.
119. The compound of any one of embodiments 108 to 111, wherein Rₕ is halogen.
120. The compound of any one of embodiments 105 to 119, wherein A is

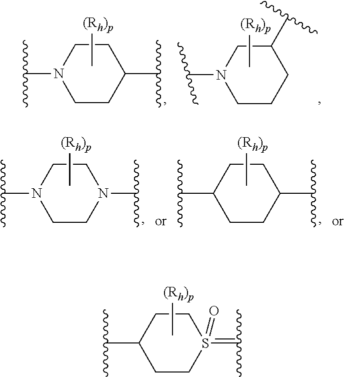

and p is 0, 1, 2, 3, or 4.
121. The compound of embodiment 120, wherein p is 0.
122. The compound of embodiment 120, wherein p is 1.
123. The compound of any one of embodiments 119 to 120, wherein A is

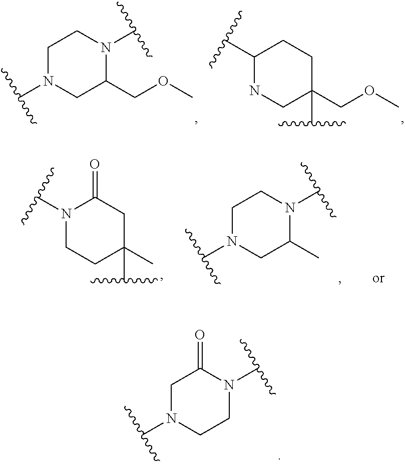

124. The compound of any one of embodiments 105 to 120, wherein A is

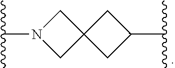 [SMILES: *C1CC2(C1)CN(*)C2]

125. The compound of any one of embodiments 105 to 120, wherein A is

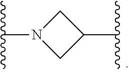 [SMILES: *C1CN(*)C1]

126. The compound of embodiments 105 to 120, wherein A is

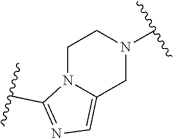 [SMILES: *c1ncc2n1CCN(*)C2]

127. The compound of any one of embodiments 105 to 126, wherein L is O, CH₂, C(═O), NRₑ or N.
128. The compound of embodiment 127, wherein Rₑ is CH₃.
129. The compound of any one of embodiments 105 to 128, wherein B is

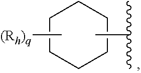

wherein q is 0, 1, 2, 3, 4, or 5.
130. The compound of any one of embodiments 105 to 128, wherein B is

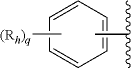

wherein q is 0, 1, 2, 3, 4, or 5.
131. The compound of embodiment 129 or 130, wherein q is 2.
132. The compound of embodiment 130, wherein B is

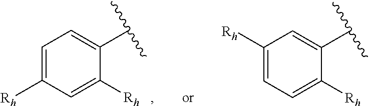

133. The compound of any one of embodiments 105 to 129, wherein B is

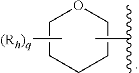

134. The compound of any one of embodiments 105 to 129, wherein B is

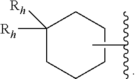

135. The compound of embodiment 133, wherein each Rₕ is independently halogen.
136. The compound of embodiment 134, wherein each Rₕ is independently F or Cl.
137. A compound selected from the group consisting of:
Compound 90-91, Compound 98, Compound 101, Compound 103, Compound 105-126, Compound 131-192, Compound 195-196, Compound 198, Compound 199-212, Compound 215, Compound 217-219, Compound 234, Compound 247, Compound 248, Compound 220, Compound 223, Compound 229, Compound 231-233, Compound 235-237, Compound 239, Compound 243-245, Compound 249, Compound 252-258, Compound 260, Compound 265-267, Compound 269, Compound 271-276, Compound 278, Compound 280-287, Compound 290-291, and Compound 299-300 from Table 1.
138. A compound of Formula (Ia), or a pharmaceutically acceptable salt thereof:

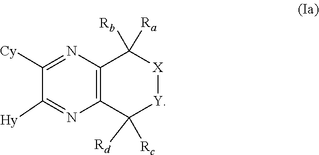

wherein
Rₐ, R_{b}, R_{c}, and R_{d}, are H;
each of X and Y is independently selected from the group consisting of O, NRₑ, N(C(Rₑ)₂C(═O)NHR_{f}), N(C(═O)Rₑ), N(C(═O)ORₑ), NC(═O)C(Rₑ)₃, S, S(═O), S(═O)₂, S(═O)(═NRₑ), S(═NRₑ)₂, NS(═O)₂Rₑ, C(CN)Rₑ, C(C(═O)Rₑ), NS(O)₂NRₑRₕ, NS(O)NRₑRₕ, C(═NSO₂Rₑ), and C(Rₕ)₂;
each Rₑ, R_{f}, and R_{g} is independently H, —OH, —CN, —OCHF₂, —CHF₂, —CF₃, —CHOH, CH(CH₃)CN, —C((CH₃)₂CN), —NHCH₃, —N(CH₃)₂, —CH₂CH(OH)CH₂OH, alkoxy, alkyl, (hetero)alkyl, (hetero)alkenyl, (hetero)alkynyl, carbocyclyl, or heterocyclyl;
Hy is a carbon-linked 3-, 4-, 5- or 6-membered carbocyclic ring or a carbon-linked 3-, 4-, 5- or 6-membered heterocyclic ring comprising one or more S or O; and
Cy is -A-L-B;
wherein
A is a saturated 4- to 7-membered hetero- or carbocyclic ring; and
B is a 5- to 7-membered hetero- or carbocyclic ring; and
L is C(Rₕ)₂, or O, and
each Rₕ is independently H, halogen, —CN, —OCHF₂, alkyl, alkoxy, —CH₂OH, —CH(OH)C(Rₑ)₃, —CH(OH)Rₑ, —NRₑR_{f}, —NRₑC(═O)R_{f}, —NRₑC(═O)NR_{f}R_{g}, —NRₑC(═O)OR_{f}, —NRₑSO₂R_{f}, —OC(═O)NRₑR_{f}, —ORₑ, —SRₑ, —SORₑ, —SO₂Rₑ, —SO₂ORₑ, —SO₂NRₑR_{f}, —C(═O)Rₑ, —C(═O)ORₑ, —C(═O)NRₑR_{f}, (hetero)alkyl, (hetero)alkenyl, (hetero)alkynyl, carbocyclyl, or heterocyclyl; or two Rₕ attached to the same carbon atom together with the atoms to which they are attached form ═NSO₂Rₑ, ═O, carbocyclyl, or heterocyclyl.
139. The compound of embodiment 137, wherein Hy is

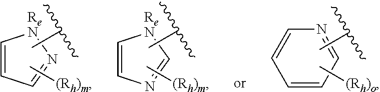

Wherein m is 0, 1, or 2; and o is 0, 1, 2, 3, or 4.
140. The compound of embodiment 138, wherein Rₑ is H or alkyl.
141. The compound of embodiment 138, wherein Rₕ is NH₂ or alkoxy.
142. The compound of any one of embodiments 137-140, wherein A is

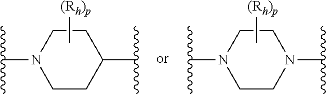

143. The compound of embodiment 141, wherein p is 0.
144. The compound of any one of embodiments 137-142, wherein B is

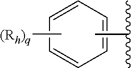

wherein q is 2.
145. The compound of embodiment 143, wherein each Rₕ is independently F or Cl.
146. A compound selected from the group consisting of:

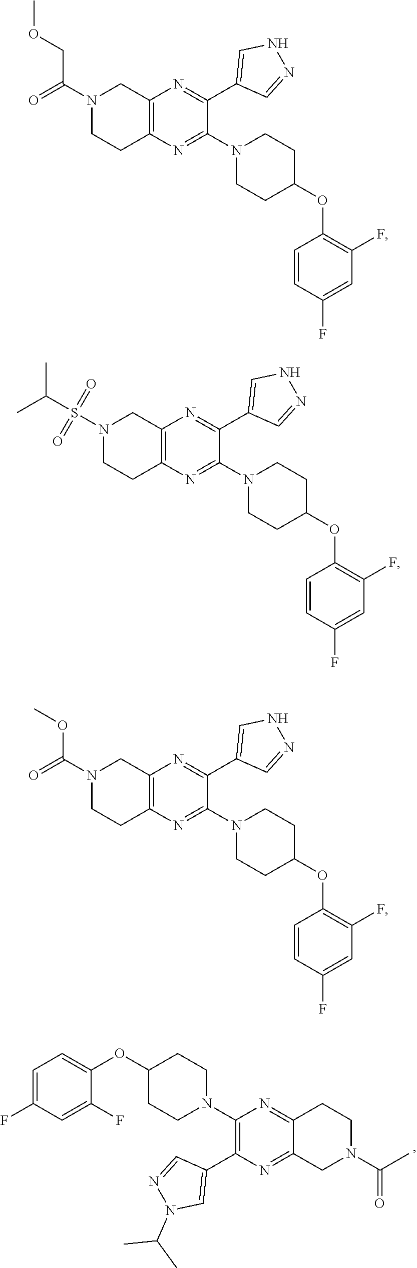 [SMILES: COCC(=O)N1CCc2nc(N3CCC(Oc4ccc(F)cc4F)CC3)c(-c3cn[nH]c3)nc2C1 ; CC(C)S(=O)(=O)N1CCc2nc(N3CCC(Oc4ccc(F)cc4F)CC3)c(-c3cn[nH]c3)nc2C1 ; COC(=O)N1CCc2nc(N3CCC(Oc4ccc(F)cc4F)CC3)c(-c3cn[nH]c3)nc2C1 ; CC(=O)N1CCc2nc(N3CCC(Oc4ccc(F)cc4F)CC3)c(-c3cnn(C(C)C)c3)nc2C1]

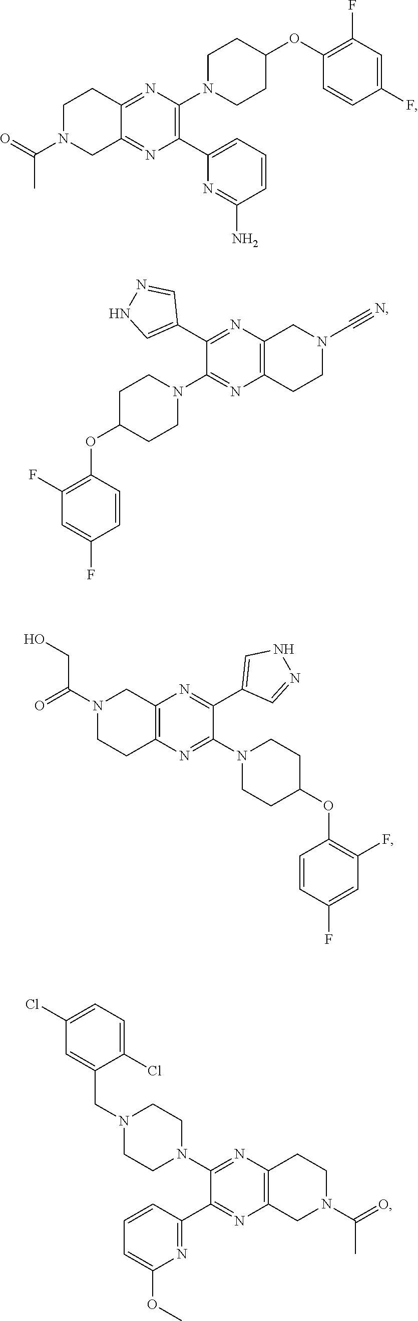 [SMILES: CC(=O)N1CCc2nc(N3CCC(Oc4ccc(F)cc4F)CC3)c(-c3cccc(N)n3)nc2C1 ; N#CN1CCc2nc(N3CCC(Oc4ccc(F)cc4F)CC3)c(-c3cn[nH]c3)nc2C1 ; O=C(CO)N1CCc2nc(N3CCC(Oc4ccc(F)cc4F)CC3)c(-c3cn[nH]c3)nc2C1 ; COc1cccc(-c2nc3c(nc2N2CCN(Cc4cc(Cl)ccc4Cl)CC2)CCN(C(C)=O)C3)n1]

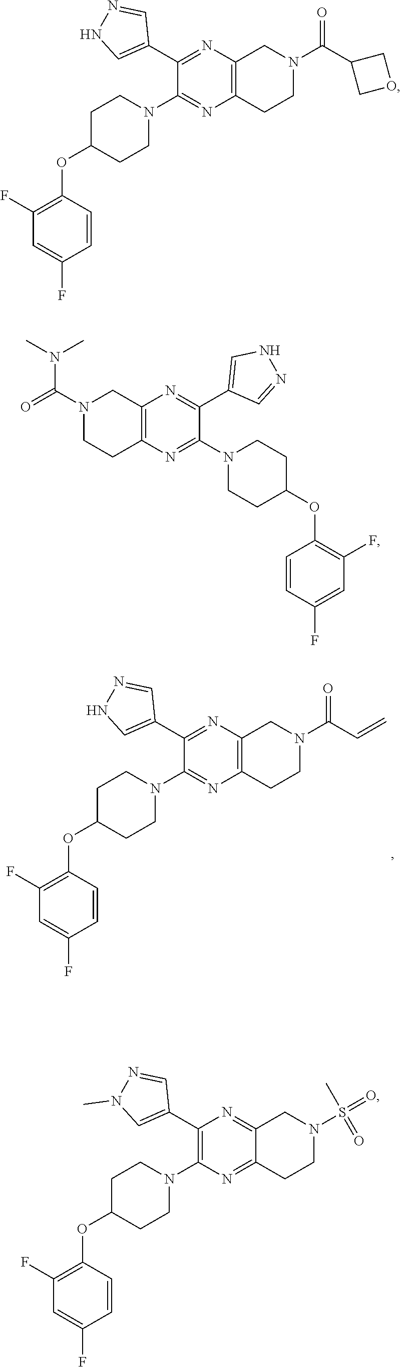 [SMILES: O=C(C1COC1)N1CCc2nc(N3CCC(Oc4ccc(F)cc4F)CC3)c(-c3cn[nH]c3)nc2C1 ; CN(C)C(=O)N1CCc2nc(N3CCC(Oc4ccc(F)cc4F)CC3)c(-c3cn[nH]c3)nc2C1 ; C=CC(=O)N1CCc2nc(N3CCC(Oc4ccc(F)cc4F)CC3)c(-c3cn[nH]c3)nc2C1 ; Cn1cc(-c2nc3c(nc2N2CCC(Oc4ccc(F)cc4F)CC2)CCN(S(C)(=O)=O)C3)cn1]

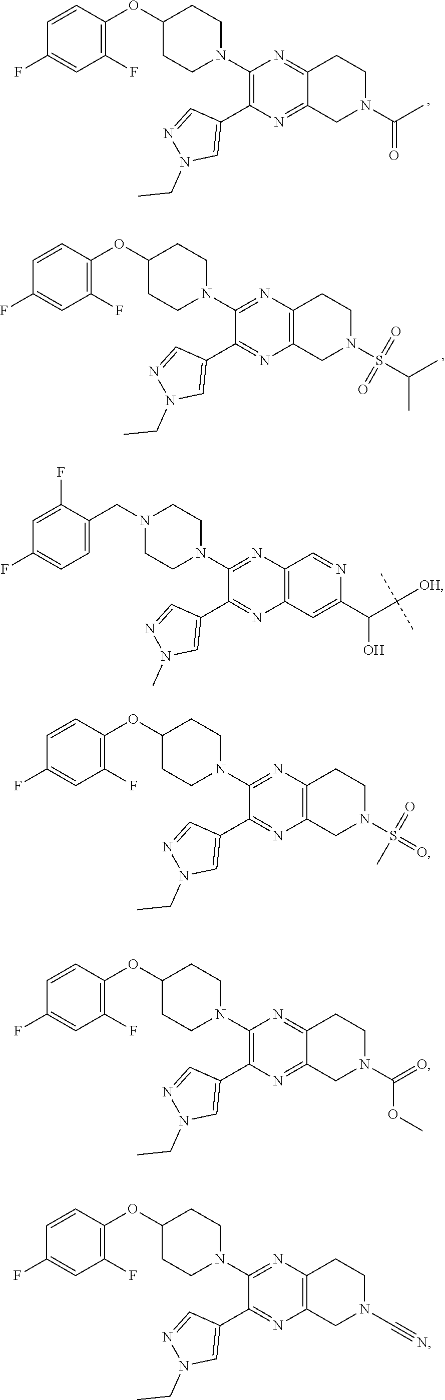

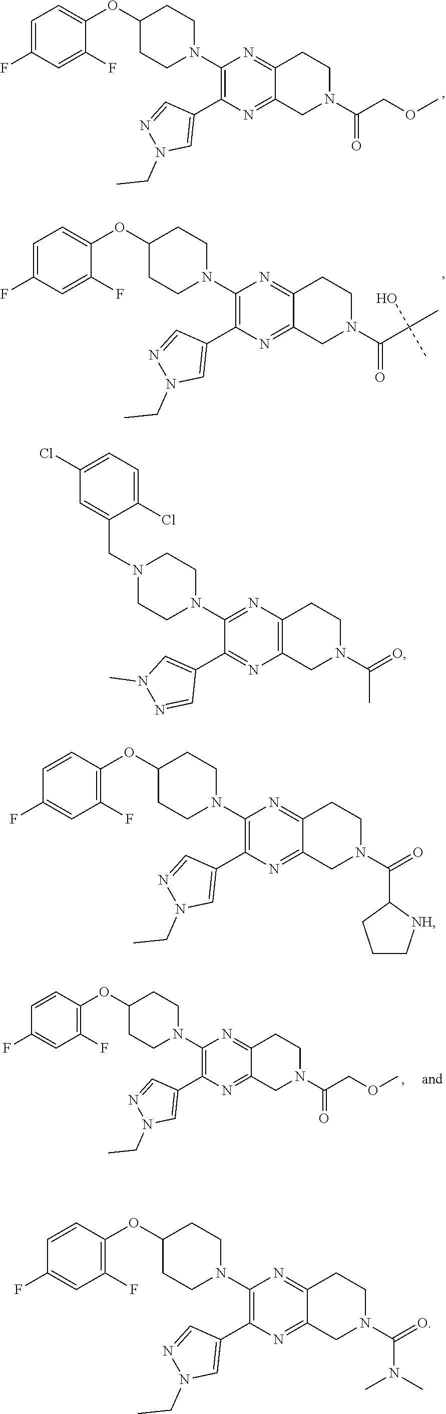

147. The compound of anyone of embodiments 1a or 1-145 wherein the compound contains one or more deuterium atoms.

### Compositions

The present disclosure provides pharmaceutical compositions for providing inverse agonism of the GPR6 receptor in a subject. In embodiments, a pharmaceutical composition comprises one or more compounds of the present disclosure (e.g., a compound of Formula (I), (I′), (Ia), (Ib), (Ic), (Id) or Table 1) or a pharmaceutically acceptable salt thereof.

In embodiments of the present disclosure, a pharmaceutical composition comprises a therapeutically effective amounts of one or more compounds of the present disclosure (e.g., a compound of Formula (I), (I′), (Ia), (Ib), (Ic), (Id) or Table 1) or a pharmaceutically acceptable salt thereof.

In embodiments, a pharmaceutical composition, as described herein, comprises one or more compounds selected from Table 1, or a pharmaceutically acceptable salt thereof.

In embodiments of the present disclosure, a pharmaceutical composition comprising one or more compounds of the present disclosure (e.g., a compound of Formula (I), (I′), (Ia), (Ib), (Ic), (Id) or Table 1) or a pharmaceutically acceptable salt thereof, and a pharmaceutically acceptable excipient or adjuvant is provided. The pharmaceutically acceptable excipients and adjuvants are added to the composition or formulation for a variety of purposes. In some embodiments, a pharmaceutical composition comprising one or more compounds disclosed herein, or a pharmaceutically acceptable salt thereof, further comprise a pharmaceutically acceptable carrier. In some embodiments, a pharmaceutically acceptable carrier includes a pharmaceutically acceptable excipient, binder, and/or diluent. In some embodiments, suitable pharmaceutically acceptable carriers include, but are not limited to, inert solid fillers or diluents and sterile aqueous or organic solutions. In some embodiments, suitable pharmaceutically acceptable excipients include, but are not limited to, water, salt solutions, alcohol, polyethylene glycols, gelatin, lactose, amylase, magnesium stearate, talc, silicic acid, viscous paraffin, and the like.

For the purposes of this disclosure, the compounds of the present disclosure can be formulated for administration by a variety of means including orally, parenterally, by inhalation spray, topically, or rectally in formulations containing pharmaceutically acceptable carriers, adjuvants and vehicles. The term parenteral as used here includes subcutaneous, intravenous, intramuscular, and intraarterial injections with a variety of infusion techniques. Intraarterial and intravenous injection as used herein includes administration through catheters.

Generally, the compounds of the present disclosure are administered in a therapeutically effective amount. The amount of the compound actually administered will typically be determined by a physician, in the light of the relevant circumstances, including the condition to be treated, the chosen route of administration, the actual compound-administered, the age, weight, and response of the individual patient, the severity of the patient's symptoms, and the like.

### Methods of Treatment

The compounds of the present disclosure find use in any number of methods. For example, in some embodiments the compounds are useful in methods for modulating a G protein-coupled receptor, e.g., G protein-coupled receptor 6 (GPR6). Accordingly, in embodiments, the present disclosure provides the use of any one of the foregoing compounds of Formula (I), (I′), (Ia), (Ib), (Ic), (Id) or Table 1 or a pharmaceutically acceptable salt thereof, for modulating G protein-coupled receptor, (e.g., GPR6) activity. For example, in embodiments modulating G protein-coupled receptor (e.g., GPR6) activity is in a mammalian cell. Modulating G protein-coupled receptor, (e.g., GPR6) activity can be in a subject in need thereof (e.g., a mammalian subject, such as a human) and for treatment of any of the described conditions or diseases.

In some embodiments, the G protein-coupled receptor, (e.g., GPR6) activity is binding. In embodiments, the G protein-coupled receptor, (e.g., GPR6) activity is inverse agonism of GPR6.

In embodiments, the present disclosure provides methods of treating a disease or disorder that is treatable by administration of an G protein-coupled receptor, (e.g., GPR6) inverse agonist, the method comprising administering a therapeutically effective amount of one or more compounds of the present disclosure (e.g., Formula (I), (I′), (Ia), (Ib), (Ic), (Id) or Table 1).

In embodiments, the compounds of the present disclosure are used for treating a variety of disorders associated with G protein-coupled receptors, including one or more of the following conditions or diseases: Parkinson's disease, dyskinesia, Huntington's disease, drug addiction, eating disorders, cognitive disorders, schizophrenia, bipolar disorder, epilepsy, Alzheimer's disease, anxiety, and depression. In embodiments, the present disclosure provides methods of treating Parkinson's disease comprising administering a therapeutically effective amount of one or more compound of Formula (I), (I′), (Ia), (Ib), (Ic), (Id). In embodiments, the present disclosure provides methods of treating Parkinson's disease comprising administering a therapeutically effective amount of one or more compound described in Table 1.

In embodiments, the compounds of the present disclosure are administered in combination with one or more other compounds used for treating a variety of disorders associated with G protein-coupled receptors, including one or more of the following conditions or diseases: Parkinson's disease, dyskinesia, Huntington's disease, drug addiction, eating disorders, cognitive disorders, schizophrenia, bipolar disorder, epilepsy, Alzheimer's disease, anxiety, and depression.

In embodiments the compounds of the present disclosure are administered in combination with one or more other compounds used for treating dyskinesia. In embodiments, the one or more other compounds used for treating dyskinesia are an N-methyl-D-aspartate (NMDA) antagonist and/or vesicular monoamine transporter 2 (VMAT2) inhibitor. In embodiments, the one or more other compounds used for treating dyskinesia are an NMDA antagonist. In embodiments, the NMDA antagonist is amantadine. In embodiments, the one or more other compounds used for treating dyskinesia are a VMAT2 inhibitor. In embodiments, the VMAT2 inhibitor is deutetrabenazine and/or valbenazine.

In embodiments the compounds of the present disclosure are administered in combination with one or more other compounds used for treating depression. In embodiments, the one or more other compounds used for treating depression are an antidepressant, atypical antidepressant, atypical antipsychotic, monoamine oxidase inhibitor (MAOI), N-methyl-D-aspartate (NMDA) antagonist, neuroactive steroid gamma-aminobutyric acid (GABA)-A receptor positive modulator, selective serotonin reuptake inhibitor (SSRI), and/or serotonin-norepinephrine reuptake inhibitor (SNRI). In embodiments, the one or more other compounds used for treating depression are an antidepressant. In embodiments, the antidepressant is amoxapine, amitriptyline, desipramine, doxepin, imipramine, lithium carbonate, maprotiline, mirtazapine, nortriptyline, protriptyline, and/or trimipramine. In embodiments, the one or more other compounds used for treating depression are an atypical antidepressant. In embodiments, the atypical antidepressant is bupropion, nefazodone and/or trazodone. In embodiments, the one or more other compounds used for treating depression are an atypical antipsychotic. In embodiments, the atypical antipsychotic is lumateperone, brexpiprazole, quetiapine, and/or olanzapine. In embodiments, the one or more other compounds used for treating depression are a MAOI. In embodiments, the MAOI is isocarboxazid, phenelzine, selegiline, and/or tranylcypromine. In embodiments, the one or more other compounds used for treating depression are an NMDA antagonist. In embodiments, the NMDA antagonist is esketamine. In embodiments, the one or more other compounds used for treating depression are a neuroactive steroid GABA-A receptor positive modulator. In embodiments, the neuroactive steroid GABA-A receptor positive modulator is brexanolone. In embodiments, the one or more other compounds used for treating depression are an SSRI. In embodiments, the SSRI is citalopram, escitalopram, fluoxetine, paroxetine, sertraline, vilazodone, and/or vortioxetine. In embodiments, the one or more other compounds used for treating depression are an SNRI. In embodiments, the SNRI is desvenlafaxine, duloxetine, levomilnacipran, and/or venlafaxine.

In embodiments the compounds of the present disclosure are administered in combination with one or more other compounds used for treating anxiety. In embodiments the one or more other compounds used for treating anxiety are an antidepressant, anxiolytic, anticonvulsant, noradrenergic agent, and/or atypical antipsychotic. In embodiments, the one or more other compounds used for treating anxiety are an antidepressant. In embodiments, the antidepressant is amitriptyline, bupro 255 lomipraminepram, clomiprimine, desipramine, duloxetine, doxepin, escitalopram, isocarboxid, fluvoxamine, fluoxetine, imipramine, maprotiline, mirtazapine, nortriptyline, paroxetine, phenelzine, protriptyline, sertraline, tranylcypromine, trazodone, trimipramine, and/or venlafaxine. In embodiments, the one or more other compounds used for treating anxiety are an anxiolytic. In embodiments, the anxiolytic is alprazolam, buspirone, chlordiazepoxide, clonazepam, clorazepate, diazepam, hydroxyzine, flurazepam, lorazepam, oxazepam, triazolam, and/or temazepam. In embodiments, the one or more other compounds used for treating anxiety are an anticonvulsant. In embodiments, the anticonvulsant is tiagabine, gabapentin, valproate, lamotrigine, and/or topiramate. In embodiments, the one or more other compounds used for treating anxiety are a noradrenergic agent. In embodiments, the noradrenergic agent is propranolol, atenolol, prazosin, prazosin, clonidine, and/or guanfacine. In embodiments, the one or more other compounds used for treating anxiety are an atypical antipsychotic. In embodiments, the atypical antipsychotic is aripiprazole, ziprasidone, risperidone, quetiapine, and/or olanzapine.

In embodiments the compounds of the present disclosure are administered in combination with one or more other compounds used for treating Parkinson's disease. In embodiments, the one or more other compounds used for treating Parkinson's disease are an adenosine A_{2A} (A2A) antagonist, anticholinergic, dopamine (DOPA) agonist, DOPA decarboxylase inhibitor, DOPA precursor, catechol-O-methyl transferase (COMT) inhibitor, monoamine Oxidase Type B (MAO-B) inhibitor, and/or N-methyl-D-aspartate (NMDA) antagonist. In embodiments, the one or more other compounds used for treating Parkinson's disease are an A2A antagonist. In embodiments, the A2A antagonist is istradefylline. In embodiments, the one or more other compounds used for treating Parkinson's disease are an anticholinergic. In embodiments, the anticholinergic is trihexyphenidyl and/or benztropine. In embodiments, the one or more other compounds used for treating Parkinson's disease are a DOPA agonist. In embodiments, the DOPA agonist is pramipexole, ropinirole, apomorphine, and/or rotigotine. In embodiments, the one or more other compounds used for treating Parkinson's disease are a DOPA decarboxylase inhibitor. In embodiments, the DOPA decarboxylase inhibitor is carbidopa. In embodiments, the one or more other compounds used for treating Parkinson's disease are a DOPA precursor. In embodiments, the DOPA precursor is levodopa. In embodiments, the one or more other compounds used for treating Parkinson's disease are a COMT inhibitor. In embodiments, the COMT inhibitor is entacapone, opicapone, and/or tolcapone. In embodiments, the one or more other compounds used for treating Parkinson's disease are a MAO-B inhibitor. In embodiments, the MAO-B inhibitor is selegiline, safinamide, and/or rasagiline. In embodiments, the one or more other compounds used for treating Parkinson's disease are an NMBA antagonist. In embodiments, the NMDA antagonist is amantadine.

### EXAMPLES

The disclosure now being generally described, it will be more readily understood by reference to the following examples which are included merely for purposes of illustration of certain aspects and embodiments of the present disclosure and are not intended to limit the invention.

The compounds of the present disclosure can be synthesized using the methods as hereinafter described below, together with synthetic methods known in the art of synthetic organic chemistry or variations thereon as appreciated by those skilled in the art.

Preparation of compounds can involve the protection and deprotection of various chemical groups. The need for protection and deprotection, and the selection of appropriate protecting groups can be readily determined by one skilled in the art. The chemistry of protecting groups can be found, for example, in Greene and Wuts, Protective Groups in Organic Synthesis, 44th. Ed., Wiley & Sons, 2006, as well as in Jerry March, Advanced Organic Chemistry, 4^{th} edition, John Wiley & Sons, publisher, New York, 1992 which are incorporated herein by reference in their entirety.

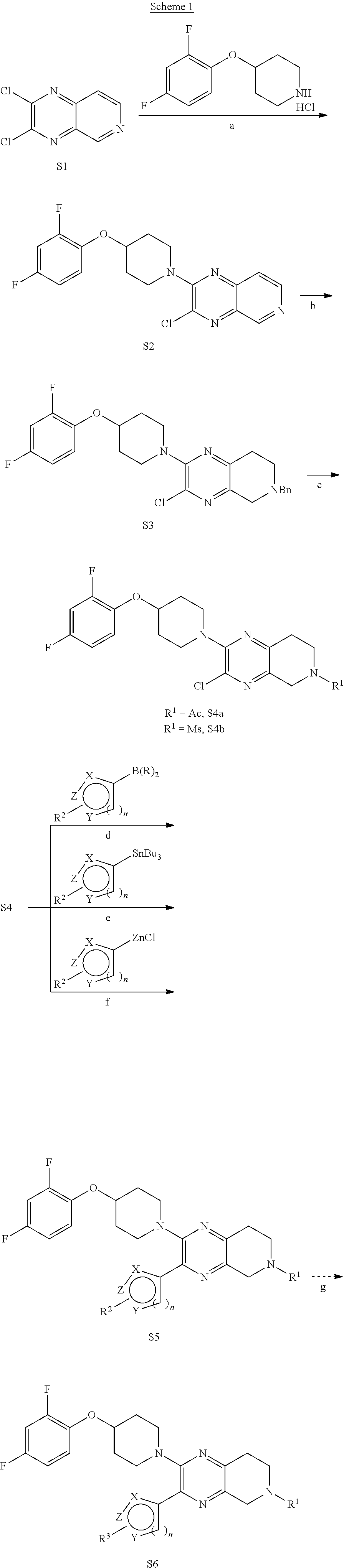

Conditions: a) 4-(2,4-difluorophenoxy)piperidine, DIPEA, DCM, 0° C. tIt; b) i. BnBr, ACN, reflux, ii. NaBH₄, MeOH, 0° C. to rt or NaBH(OAc)₃, DCE; c) i. 1-chloroethyl chloroformate (CECF), 1,2-DCE, reflux, ii. MeOH, reflux. Ac₂, DIPEA, DCM, rt or MsCl, DIPEA, DCM, rt; d) heteroaryl boronic acid or heteroaryl pinacol boronic ester, Pd(PPh₃)₄ or Pd(dppf)Cl₂·CH₂Cl₂, K₃PO₄, dioxane/water, 110° C.; e) (tributylstannyl)heteroaryl, Pd(PPh₃)₄ or Pd(dppf)Cl₂·CH₂Cl₂, toluene, 110° C.; f) i. Arylbromide, iPrMgBr, ZnCl, THF, rt, ii. S4, Pd(PPh₃)₄, 60° C.; g) deprotection and/or additional modifications (not in all cases).

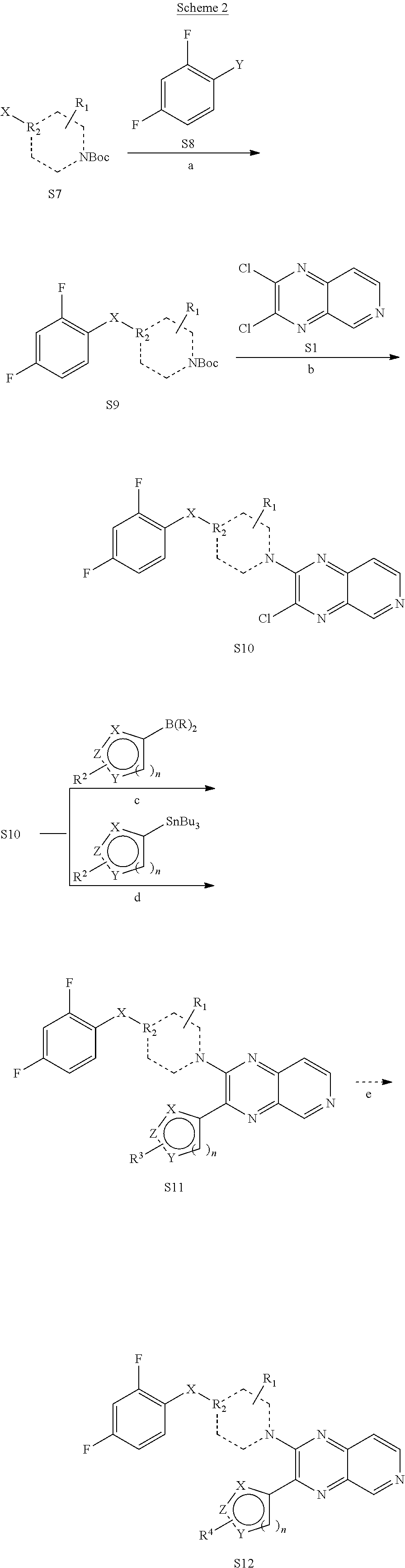

Conditions: a) initial core modifications, see procedures for further details b) i. TFA, DCM, rt, ii. 2,3-dichloropyrido[3,4-b]pyrazine (S1), DIPEA, DCM, 0° C. rt; c) heteroaryl boronic acid or heteroaryl pinacol boronic ester, Pd(PPh₃)₄ or Pd(dppf)Cl₂·CH₂Cl₂, K₃PO₄, dioxane/water, 110° C.; d) (tributylstannyl)heteroaryl, Pd(PPh₃)₄ or Pd(dppf)Cl₂·CH₂Cl₂, toluene, 110° C.; f) deprotection and/or additional modifications (not in all cases).

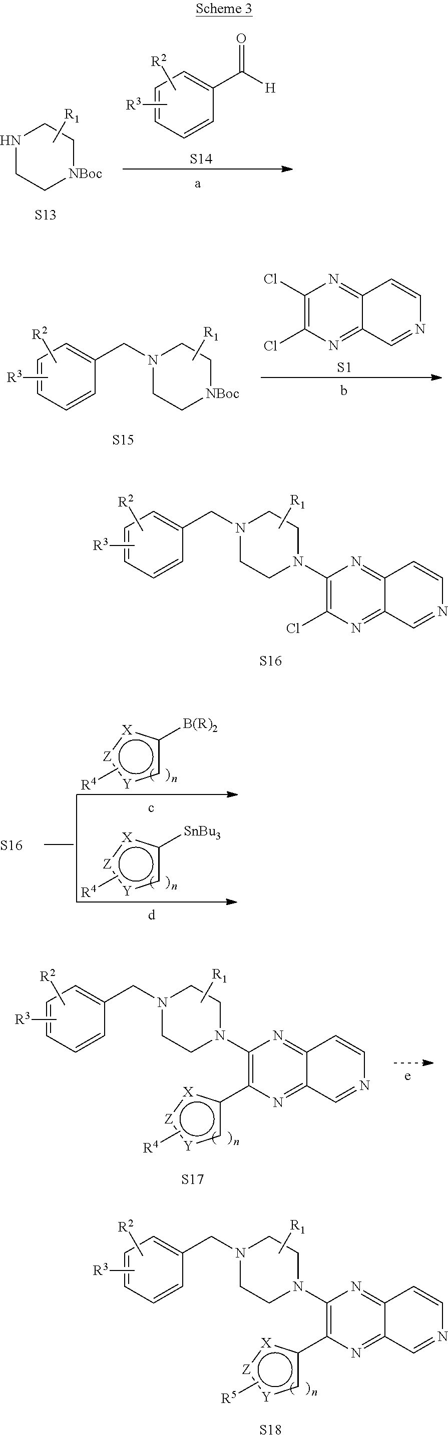

Conditions: a) benzaldehyde (S14), Sodium triacetoxyborohydride, 1,2-DCE, rt; b) i. HCl, dioxane, rt, ii. 2,3-dichloropyrido[3,4-b]pyrazine (S1), DIPEA, DCM, 0° C. rt; c) heteroaryl boronic acid or heteroaryl pinacol boronic ester, Pd(PPh₃)₄ or Pd(dppf)Cl₂·CH₂Cl₂, K₃PO₄, dioxane/water, 110° C.; d) (tributylstannyl)heteroaryl, Pd(PPh₃)₄ or Pd(dppf)Cl₂·CH₂Cl₂, toluene, 110° C.; e) deprotection and/or additional modifications (not in all cases).

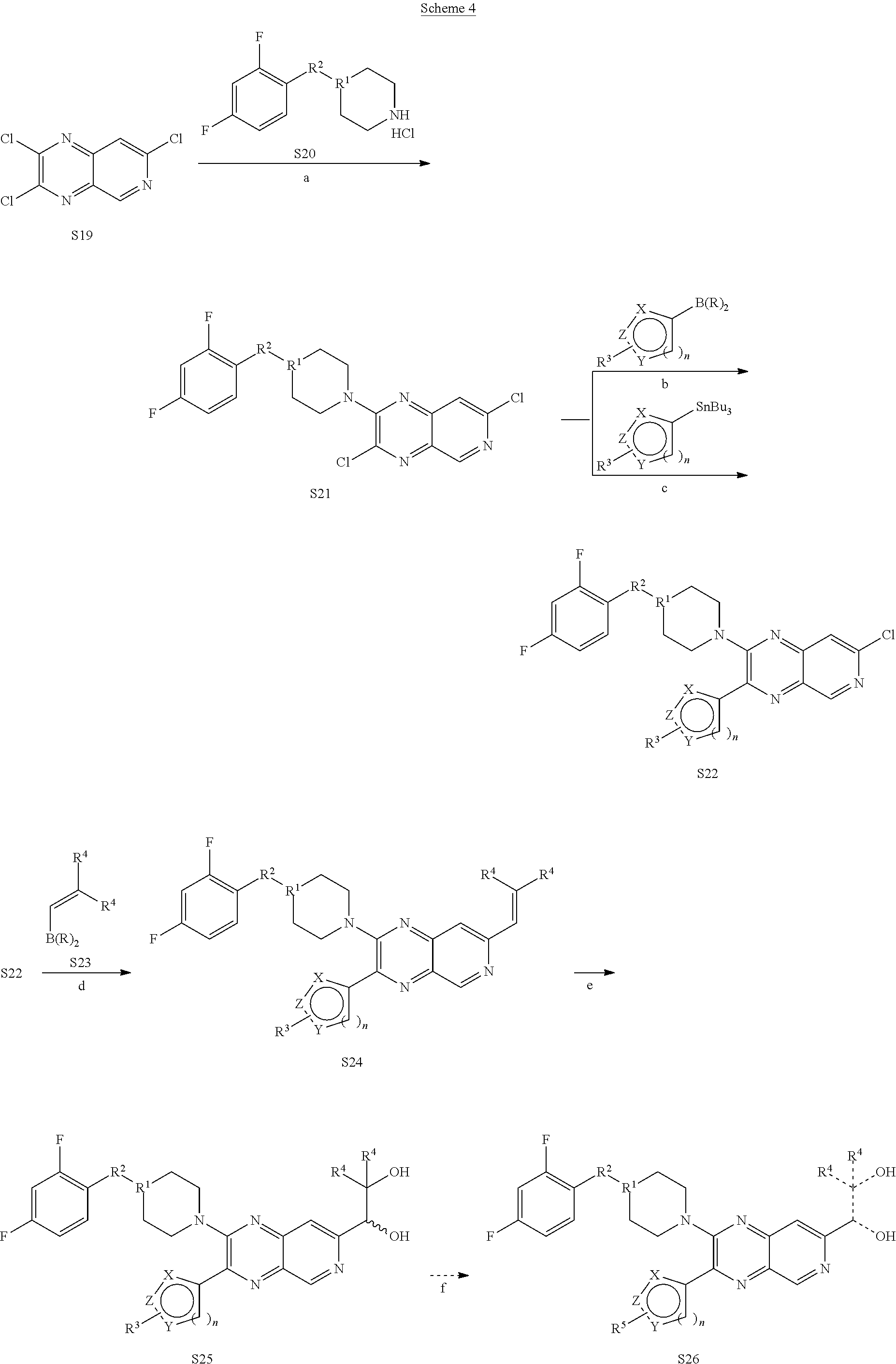

Conditions: a) piperidine or piperazine (S23), DIPEA, DCM, 0° C.; b) heteroaryl boronic acid or heteroaryl pinacol boronic ester, Pd(PPh₃)₄ or Pd(dppf)Cl₂·CH₂Cl₂, K₃PO₄, dioxane/water, 110° C.; c) (tributylstannyl)heteroaryl, Pd(PPh₃)₄ or Pd(dppf)Cl₂·CH₂Cl₂, toluene, 110° C.; d) Alkenyl boronic acid or pinacol boronic ester (S27), Pd(dppf)Cl₂·CH₂Cl₂, K₃PO₄, dioxane/water, 110° C.; e) NMO, OsO₄, THF/H₂O, rt; f) deprotection and/or additional modifications (not in all cases).

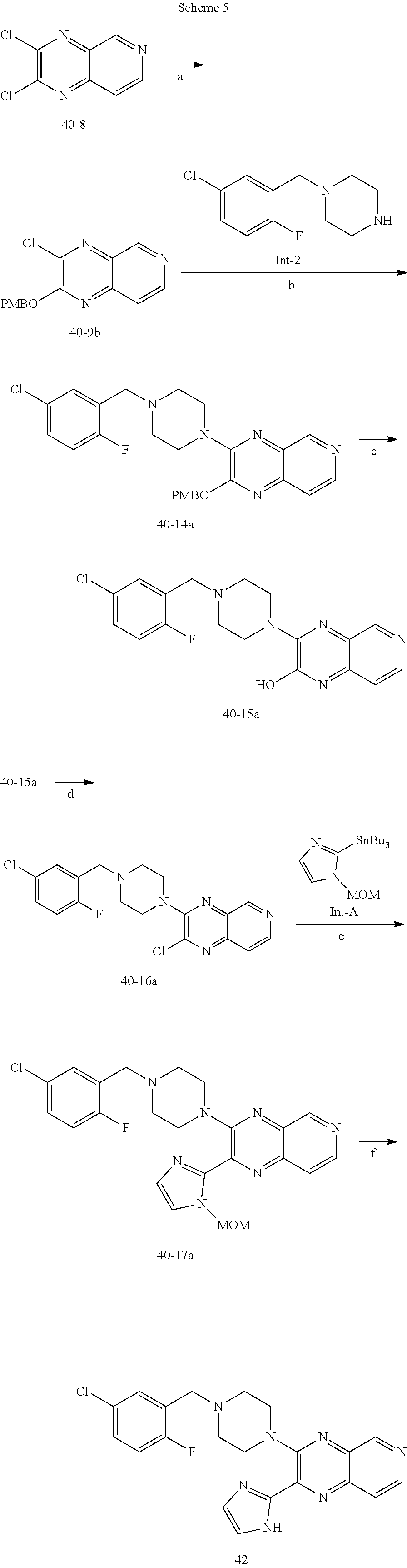

Conditions: a) n-BuLi, PMB-OH, −78° C. to rt; b) DIPEA, Dioxane, 80° C., 6h; c) TFA, DCM, rt; d) SOCl₂, DMF, reflux, 16h; e) Int-A, Pd(PPh₃)₄, toluene, 110° C.; f) TFA, reflux, 16h.

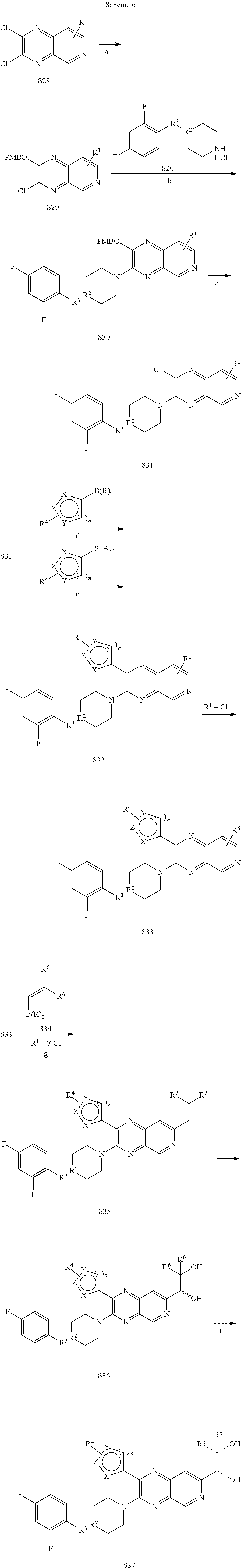

Conditions: a) PMBOH, DIPEA, DCE, 40° C.; b) piperidine or piperazine (S20), DIPEA, KF, DMSO, rt; c) i. TFA, DCM, rt, ii. SOCl2, DMF, reflux; d)heteroaryl boronic acid or heteroaryl pinacol boronic ester, Pd(PPh₃)₄ or Pd(dppf)Cl₂·CH₂Cl₂, K₃PO₄, Dioxane/water, 110° C.; e) (tributylstannyl)heteroaryl, Pd(PPh₃)₄ or Pd(dppf)Cl₂·CH₂Cl₂, Toluene, 110° C.; f) modifications into the halogen position; g) Alkenyl boronic acid or pinacol boronic ester (S34), Pd(dppf)Cl₂·CH₂Cl₂, K₃PO₄, Dioxane/water, 110° C.; b) NMO, OsO₄, THF/H₂O, rt; i) deprotection and/or additional modifications (not in all cases)

The compounds of the present disclosure (e.g., Table 1) can be prepared using, for example, methods described in Scheme 1, Scheme 2, Scheme 3, Scheme 4 or Scheme 5. Table 2 provides characterization data (e.g., NMR and Mass Spectrometry) for example compounds of the present disclosure. The GPR6 and GPR3 IC50 data was obtained using the procedure as described in Example 24.

TABLE 2
Characterization for compounds of the present disclosure

| | GPR6 | GPR3 | ESI + ve | |
|---|---|---|---|---|
| | IC50 | IC50 | [M + H] | 1H NMR |
| Cmpd | | | | |
| 1 | ** | *** | 456.2 | 1H NMR (400 MHz, DMSO-d6) δ 8.32 (d, J = 8.6 |
| | | | | Hz, 1H), 7.27 (ddd, J = 11.9, 7.3, 3.2 Hz, 2H), 7.01- |
| | | | | 6.95 (m, 1H), 4.64 (d, J = 12.8 Hz, 2H), 4.50 (s, 1H), |
| | | | | 3.81-3.78 (m, 2H), 3.49-3.38 (m, 2H), 3.02-2.98 |
| | | | | (m, 3H), 2.94 (t, J = 5.6 Hz, 2H), 2.80 (t, J = 5.7 Hz, |
| | | | | 1H), 2.13 (d, J = 10.6 Hz, 3H), 1.94 (s, 2H), 1.67- |
| | | | | 1.63 (m, 2H). |
| 2 | *** | ** | 491.5 | 1H NMR (400 MHz, DMSO-d6) δ 8.26-8.14 (m, |
| | | | | 2H), 8.09-8.02 (m, 1H), 7.33-7.21 (m, 2H), 7.04- |
| | | | | 6.94 (m, 1H), 4.65 (d, J = 21.6 Hz, 2H), 4.55-4.44 |
| | | | | (m, 1H), 3.84-3.77 (m, 2H), 3.46-3.36 (m, 2H), |
| | | | | 3.11-2.98 (m, 2H), 2.95 (t, J = 5.7 Hz, 1H), 2.82 (t, |
| | | | | J = 5.6 Hz, 1H), 2.14 (s, 1.5H), 2.11 (s, 1H), 1.98- |
| | | | | 1.79 (m, 2H), 1.68-1.53 (m, 2H). |
| 3 | *** | ** | 519.5 | 1H NMR (400 MHz, DMSO-d6) δ 8.46 (s, 1H), 8.13 |
| | | | | (d, J = 2.8 Hz, 1H), 7.39-7.21 (m, 2H), 7.01 (t, J = |
| | | | | 8.6 Hz, 1H), 6.38 (td, J = 54.9, 27.4 Hz, 1H), 4.83- |
| | | | | 4.70 (m, 2H), 4.63 (d, J = 17.0 Hz, 2H), 4.56-4.46 |
| | | | | (m, 1H), 3.85-3.71 (m, 2H), 3.33-3.26 (m, 2H), |
| | | | | 3.04-2.84 (m, 3H), 2.77 (t, J = 5.5 Hz, 1H), 2.16- |
| | | | | 2.00 (m, 5H), 1.93-1.75 (m, 2H). |
| 4 | *** | ** | 424.4 | 1H NMR (400 MHz, DMSO-d6) δ 13.32 (bs, 1H), |
| | | | | 9.12 (s, 1H), 8.55 (d, J = 5.7 Hz, 1H), 8.47 (bs, 1H), |
| | | | | 8.20 (bs, 1H), 7.62 (d, J = 5.6 Hz, 1H), 7.52-7.47 |
| | | | | (m, 1H), 7.42-7.36 (m, 1H), 7.25 (t, J = 9.1 Hz, 1H), |
| | | | | 3.59 (s, 2H), 3.44-3.34 (m, 4H, overlapping), 2.62- |
| | | | | 2.55 (m, 4H, overlapping). |
| 5 | *** | ** | 440.3 | 1H NMR (400 MHz, DMSO-d6): 12.84 (bs, 1H), 9.10 |
| | | | | (s, 1H), 8.54 (d, J = 5.6 Hz, 1H), 7.88 (dd, J = 0.8 Hz, |
| | | | | J = 0.8 Hz, 2H), 7.60-7.57 (m, J = 8.0 Hz, 2H), 7.49 |
| | | | | (d, J = 8.4 Hz, 1H), 7.39 (dd, J = 2.8 Hz, J = 2.8 Hz, 2H), |
| | | | | 3.62 (s, 2H), 3.41 (bs, 4H), 2.61 (bs, 4H). |
| 6 | ** | ** | 540.2 | 1H NMR (400 MHz, CDCl3): 8.25 (d, J = 2.0 Hz, 1H), |
| | | | | 8.01 (dd, J = 2.4 Hz, J = 2 Hz, 1H), 7.84 (s, 1H), 7.82 (d, |
| | | | | J = 9.2 Hz, 1H), 7.79 (s, 1H), 7.52 (d, J = 2.8 Hz, 1H), |
| | | | | 7.29 (s, 1H), 7.18 (dd, J = 2.8 Hz, J = 2.4 Hz, 1H), 3.63 |
| | | | | (s, 2H), 3.43 (bt, J = 3.6 Hz, 4H), 3.26 (s, 3H), 3.08 (d, |
| | | | | J = 4.8 Hz, 3H), 2.67 (bt, J = 4.8 Hz, 4H). |
| 7 | ** | *** | 496.4 | 1H NMR (400 MHz, DMSO-d6) δ 8.43 (dd, J = 5.7, |
| | | | | 2.2 Hz, 1H), 7.30 (d, J = 2.4 Hz, 1H), 7.27-7.17 (m, |
| | | | | 2H), 6.96 (dd, J = 5.1, 2.8 Hz, 1H), 6.95-6.90 (m, |
| | | | | 1H), 4.58 (d, J = 19.2 Hz, 2H), 4.45-4.34 (m, J = 11.7, |
| | | | | 4.0 Hz, 1H), 3.84 (s, 3H), 3.76 (dd, J = 10.9, 5.5 Hz, |
| | | | | 2H), 3.43-3.34 (m, 2H), 3.03-2.90 (m, 2H), 2.88 (t, |
| | | | | J = 5.8 Hz, 1H), 2.75 (t, J = 5.7 Hz, 1H), 2.09 (s, |
| | | | | 1.5H), 2.07 (s, 1.5H), 1.89-1.74 (m, 2H), 1.58-1.41 |
| | | | | (m, 2H). |
| 8 | *** | ** | 472.3 | 1H NMR (400 MHz, DMSO-d6) δ 9.58 (d, J = 1.5 |
| | | | | Hz, 1H), 9.12 (d, J = 3.5 Hz, 1H), 7.33-7.24 (m, |
| | | | | 2H), 7.03-6.96 (m, 1H), 4.66 (d, J = 17.4 Hz, 2H), |
| | | | | 4.50 (dt, J = 8.0, 4.1 Hz, 1H), 3.81 (dd, J = 9.0, 5.7 |
| | | | | Hz, 2H), 3.43-3.25 (m, 2H, overlapping), 3.02- |
| | | | | 2.90 (m, 3H), 2.81 (t, J = 5.8 Hz, 1H), 2.14 (s, 1.5H), |
| | | | | 2.12 (s, 1.5H), 2.06-1.96 (m, 2H), 1.85-1.71 (m, 2H). |
| 9 | *** | *** | 409.31 | 1H NMR (400 MHz, DMSO-d6) δ 13.3 (bs, 1H), 9.12 |
| | | | | (s, 1H), 8.55 (d, J = 5.7 Hz, 1H), 8.54-8.40 |
| | | | | (overlapping m, 1H), 8.32-8.13 (m, 1H), 7.62 (dd, |
| | | | | J = 5.7, 0.4 Hz, 1H), 7.36-7.23 (m, 2H), 7.06-6.95 |
| | | | | (m, 1H), 4.57 (dt, J = 7.8, 4.0 Hz, 1H), 3.65 (dd, J = |
| | | | | 10.1, 6.0 Hz, 2H), 3.28-3.20 (overlapping m, 2H), |
| | | | | 2.13-2.00 (m, 2H), 1.90-1.72 (m, 2H). |
| 10 | *** | ** | 495.52 | 1H NMR (400 MHz, DMSO-d6) δ 8.38 (t, J = 2.5 Hz, |
| | | | | 1H), 7.81 (d, J = 8.7 Hz, 1H), 7.55-7.48 (m, 1H), |
| | | | | 7.32-7.18 (m, 2H), 7.03-6.92 (m, 1H), 4.61 (d, J = |
| | | | | 19.8 Hz, 2H), 4.49-4.41 (m, 1H), 3.88 (s, 3H), 3.86- |
| | | | | 3.78 (m,, 2H), 3.41-3.36 (m, 2H), 2.99-2.96 (m, |
| | | | | 2H, overlapped), 2.91 (t, J = 5.6 Hz, 1H, overlapped), |
| | | | | 2.78 (t, J = 5.6 Hz, 1H), 2.13 (s, 1.5H), 2.10 (s, 1.5H), |
| | | | | 1.89-1.86 (m, 2H), 1.61-1.54 (m, 2H). |
| 11 | ** | ** | 455.3 | 1H NMR (400 MHz, DMSO-d6) δ 13.20 (s, 1H), 7.66 |
| | | | | (d, J = 24.6 Hz, 1H), 7.35-7.19 (m, 1H), 7.06-6.95 |
| | | | | (m, 1H), 6.83 (s, 1H), 4.64 (d, J = 11.8 Hz, 1H), 4.50 |
| | | | | (s, 1H), 3.87-3.76 (m, 1H), 3.40 (dd, J = 37.7, 10.0 |
| | | | | Hz, 2H), 3.05-2.99 (m, 2H), 2.95 (t, J = 5.6 Hz, 1H), |
| | | | | 2.12 (d, J = 7.4 Hz, 3H), 2.00 (d, J = 7.2 Hz, 2H), |
| | | | | 1.76 (d, J = 8.0 Hz, 2H). |
| 12 | ** | ** | 483.3 | 1H NMR (400 MHz, DMSO-d6) δ 11.94 (bs, 1H, |
| | | | | NH), 8.16-7.97 (m, 2H)*, 7.35-7.19 (m, 2H), 7.07- |
| | | | | 6.89 (m, 1H), 4.66 (s, 1H), 4.63 (s, 1H), 4.50 (dd, |
| | | | | J = 7.8, 3.9 Hz, 1H), 3.81 (dd, J = 10.1, 5.3 Hz, 2H), |
| | | | | 3.51-3.39 (m, 2H) **, 3.14-3.02 (m, 2H), 2.95 (t, |
| | | | | J = 5.8 Hz, 1H), 2.82 (t, J = 5.8 Hz, 1H), 2.13 (s, 1.5H), |
| | | | | 2.11 (s, 1.5H), 2.00-1.88 (m, 2H), 1.76-1.61 (m, 2H). |
| 13 | ** | *** | 467.3 | 1H NMR (400 MHz, DMSO-d6) δ 9.26-9.20 (m, |
| | | | | 1H), 8.87 (dd, J = 5.2, 3.3 Hz, 1H), 7.94 (ddd, J = 4.5, |
| | | | | 3.1, 1.3 Hz, 1H), 7.28-7.16 (m, 2H), 6.95 (ddd, J = |
| | | | | 4.7, 3.8, 1.7 Hz, 1H), 4.61 (d, J = 18.7 Hz, 2H), 4.46 |
| | | | | (dt, J = 8.3, 4.0 Hz, 1H), 3.77 (dd, J = 9.6, 5.7 Hz, |
| | | | | 2H), 3.43 (d, J = 14.2 Hz, 2H), 3.04 (t, J = 10.4 Hz, |
| | | | | 2H), 2.91 (t, J = 5.8 Hz, 1H), 2.78 (t, J = 5.8 Hz, 1H), |
| | | | | 2.10 (s, 1.5H), 2.07 (s, 1.5H), 1.95-1.81 (m, 2H), |
| | | | | 1.68-1.46 (m, 2H). |
| 14 | *** | ** | 425.4 | 1H NMR (400 MHz, DMSO-d6) δ 8.53 (s, 1H), 8.15 |
| | | | | (d, J = 2.9 Hz, 1H), 7.49-7.20 (m, 2H), 7.13-6.94 |
| | | | | (m, 1H), 5.48 (s, 2H), 4.64 (d, J = 16.9 Hz, 2H), 4.58- |
| | | | | 4.46 (m, 1H), 3.88-3.74 (m, 2H), 3.33 |
| | | | | (overlapping m, 2H), 3.26 (s, 3H), 2.97 (t, J = 10.2 |
| | | | | Hz, 2H), 2.91 (t, J = 5.7 Hz, 1H), 2.78 (t, J = 5.6 Hz, |
| | | | | 1H), 2.13 (s, 1.5H), 2.12 (s, 1.5H), 2.10-2.02 (m, |
| | | | | 2H), 1.92-1.78 (m, 2H). |
| 15 | *** | ** | 505.31 | 1H NMR (400 MHz, DMSO-d6) δ 8.79 (s, 1H), 8.38 |
| | | | | (d, J = 4.8 Hz, 1H), 7.94 (td, J = 58.7, 3.2 Hz, 1H), |
| | | | | 7.35-7.25 (m, 2H), 7.05-6.96 (m, 1H), 4.66 (d, J = |
| | | | | 16.4 Hz, 2H), 4.57-4.47 (m, 1H), 3.85-3.75 (m, |
| | | | | 2H), 3.40-3.26 (overlapping m, 2H), 3.06-2.90 |
| | | | | (overlapping t's, 3H), 2.80 (t, J = 5.7 Hz, 1H), 2.13 (s, |
| | | | | 1.5H), 2.11 (s, 1.5H), 2.11-2.01 (m, 2H), 1.91-1.77 |
| | | | | (m, 2H). |
| 16 | *** | ** | 501.3 | 1H NMR (400 MHz, DMSO-d6) δ 8.42 (s, 1H), 8.11 |
| | | | | (d, J = 1.9 Hz, 1H), 7.34-7.24 (m, 2H), 7.05-6.96 |
| | | | | (m, 1H), 4.80 (dt, J = 47.3, 4.6 Hz, 2H), 4.63 (d, J = |
| | | | | 16.7 Hz, 2H), 4.60-4.55 (m, 1H), 4.54-4.45 (m, |
| | | | | 2H), 3.82-3.72 (m, 2H), 3.36-3.26 (m, 2H, |
| | | | | overlapping), 3.01-2.83 (m, 3H), 2.77 (t, J = 5.6 Hz, |
| | | | | 1H), 2.13 (s, 1.5H), 2.11 (s, 1, 5H), 2.10-2.01 (m, |
| | | | | 2H), 1.90-1.76 (m, 2H). |
| 17 | ** | ** | 552.4 | 1H NMR (400 MHz, DMSO-d6) δ 7.63 (s, 0.7H), |
| | | | | 7.61 (s, 0.3H), 7.33-7.24 (m, 2H), 7.04-6.96 (m, |
| | | | | 1H), 4.82 (s, 1H), 4.70 (s, 1H), 4.62 (d, J = 13.0 Hz, |
| | | | | 2H), 4.54-4.45 (m, 1H), 4.36-4.28 (m, 1H), 4.20 (t, |
| | | | | J = 4.9 Hz, 1H), 3.94-3.84 (m, 2H), 3.84-3.75 (m, |
| | | | | 2H), 3.44-3.35 (m, 2H), 3.00 (t, J = 10.5 Hz, 2H), |
| | | | | 2.91 (t, J = 5.4 Hz, 1H), 2.78 (t, J = 5.6 Hz, 1H), 2.17- |
| | | | | 2.09 (m, 6H), 2.03-1.93 (m, 2H), 1.77-1.65 |
| | | | | (m, 2H). |
| 18 | ** | *** | 456.4 | 1H NMR (400 MHz, DMSO-d6) δ 8.29 (d, J = 0.7 |
| | | | | Hz, 1H), 7.47 (dd, J = 2.9, 0.6 Hz, 1H), 7.33-7.24 |
| | | | | (m, 2H), 7.03-6.96 (m, 1H), 4.64 (d, J = 22.2 Hz, |
| | | | | 2H), 4.58-4.50 (m, 1H), 3.84-3.77 (m, 2H), 3.54- |
| | | | | 3.44 (m, 2H), 3.14-3.05 (m, 2H), 2.95 (t, J = 5.8 Hz, |
| | | | | 1H), 2.81 (t, J = 5.8 Hz, 1H), 2.13 (s, 1.5H), 2.11 (s, |
| | | | | 1.5H), 2.02-1.94 (m, 2H), 1.76-1.65 (m, 2H). |
| 19 | ** | *** | 467.4 | 1H NMR (400 MHz, DMSO-d6) δ 9.10 (d, J = 1.5 |
| | | | | Hz, 1H), 8.79-8.70 (m, 1H), 8.65 (t, J = 2.2 Hz, 1H), |
| | | | | 7.25 (ddt, J = 7.4, 5.5, 3.7 Hz, 2H), 7.05-6.93 (m, |
| | | | | 1H), 4.65 (d, J = 19.3 Hz, 2H), 4.52-4.44 (m, 1H), |
| | | | | 3.81 (dd, J = 10.1, 5.7 Hz, 2H), 3.41 (overlapping m, |
| | | | | 2H), 3.08-2.99 (m, 2H), 2.95 (t, J = 5.8 Hz, 1H), |
| | | | | 2.82 (t, J = 5.8 Hz, 1H), 2.14 (s, 1.5H), 2.11 (s, 1.5H), |
| | | | | 1.89 (dt, J = 9.2, 7.4 Hz, 2H), 1.65-1.53 (m, 2H). |
| 20 | *** | ** | 496.4 | 1H NMR (400 MHz, DMSO-d6) δ 8.28 (dd, J = 5.2, |
| | | | | 2.2 Hz, 1H), 7.47 (d, J = 5.4 Hz, 1H), 7.32-7.21 (m, |
| | | | | 3H), 7.03-6.95 (m, 1H), 4.65 (d, J = 18.7 Hz, 2H), |
| | | | | 4.48 (tt, J = 7.5, 3.7 Hz, 1H), 3.90 (s, 3H), 3.80 (dd, |
| | | | | J = 9.2, 5.4 Hz, 2H), 3.45-3.35 (m, 2H), 3.05-2.96 |
| | | | | (overlapping m, 2H), 2.94 (overlapping t, J = 6.0 Hz, |
| | | | | 1H), 2.81 (t, J = 5.5 Hz, 1H), 2.13 (s, 1.5H), 2.11 (s, |
| | | | | 1.5H), 2.02-1.85 (m, 2H), 1.73-1.59 (m, 2H). |
| 21 | ** | ** | 496.4 | 1H NMR (400 MHz, DMSO-d6) δ 8.32 (t, J = 3.1 Hz, |
| | | | | 1H), 8.06 (dd, J = 9.5, 2.5 Hz, 1H), 7.32-7.22 (m, |
| | | | | 2H), 7.03-6.94 (m, 1H), 6.50 (dd, J = 9.5, 3.7 Hz, |
| | | | | 1H), 4.63 (d, J = 12.9 Hz, 2H), 4.48 (dt, J = 7.7, 4.0 |
| | | | | Hz, 1H), 3.79 (q, J = 5.6 Hz, 2H), 3.53 (s, 3H), 3.44- |
| | | | | 3.36 (overlapping m, 2H), 3.06-2.95 (m, 2H), 2.91 |
| | | | | (t, J = 5.7 Hz, 1H), 2.78 (t, J = 5.7 Hz, 1H), 2.13 (s, |
| | | | | 1.5H), 2.10 (s, 1.5H), 2.04-1.95 (m, 2H), 1.78-1.63 (m). |
| 22 | * | *** | 468.4 | 1H NMR (400 MHz, DMSO-d6) δ 13.31 (s, 1H), 9.12 |
| | | | | (s, 1H), 8.55 (d, J = 5.7 Hz, 1H), 8.48 (s, 1H), 8.20 (s, |
| | | | | 1H), 7.80-7.72 (m, 3H), 7.64-7.60 (m, 1H), 3.69 |
| | | | | (s, 2H), 3.38 (s, 4H), 3.27 (s, 3H), 2.61 (s, 4 Hz). |
| 23 | ** | *** | 538.2 | 1H NMR (400 MHz, DMSO-d6) δ 7.88-7.73 (m, |
| | | | | 1H), 7.36 (d, J = 7.3 Hz, 0.3 H), 7.30-7.25 (m, 2H), |
| | | | | 7.00-6.97 (m, 1H), 6.82 (d, J = 8.3 Hz, 0.3 H), 6.68 |
| | | | | (d, J = 8.3 Hz, 0.6H), 4.61 (d, J = 18.8 Hz, 2H), 4.47 |
| | | | | (bs, 1H), 4.06 (d, J = 6.7 Hz, 0.6H), 3.81 (bs, 2H), |
| | | | | 3.43 (d, J = 11.7 Hz, 2H), 3.05-2.88 (m, 2H), 2.80 |
| | | | | (s, 0.6H), 2.18-2.04 (m, 3H), 1.87-1.80 (m, 2H), |
| | | | | 1.53 (s, 8H), 0.97 (d, J = 6.7 Hz, 2H). |
| 24 | ** | *** | 510.5 | 1H NMR (400 MHz, DMSO-d6) δ 7.53 (d, J = 5.4 |
| | | | | Hz, 1H), 7.33-7.23 (m, 2H), 7.05-6.95 (m, 1H), |
| | | | | 4.61 (d, J = 16.5 Hz, 2H), 4.53-4.45 (m, 1H), 4.11 |
| | | | | (t, J = 5.0 Hz, 2H), 3.92 (s, 2H), 3.79 (q, J = 5.6 Hz, |
| | | | | 2H), 3.44-3.34 (overlapped m, 3H), 3.05 (t, J = 4.8 |
| | | | | Hz, 2H), 3.03-2.94 (m, 2H), 2.90 (t, J = 5.5 Hz, 1H), |
| | | | | 2.77 (t, J = 5.7 Hz, 1H), 2.13 (s, 1.7H), 2.11 (s, 1.3H), |
| | | | | 2.02-1.93 (m, 2H), 1.76-1.65 (m, 2H). |
| 25 | ** | ** | 526.4 | 1H NMR (400 MHz, DMSO-d6) δ 8.39 (s, 1H), 8.09- |
| | | | | 8.00 (m, 2H), 7.29 (tt, J = 5.1, 4.5 Hz, 2H), 7.05- |
| | | | | 6.96 (m, 1H), 4.87 (s, 2H), 4.63 (d, J = 17.7 Hz, 2H), |
| | | | | 4.55-4.47 (m, 1H), 3.79 (dd, J = 9.9, 5.6 Hz, 2H), |
| | | | | 3.26 (overlapping m, 3H), 2.93 (dt, J = 11.8, 8.2 Hz, |
| | | | | 3H), 2.77 (t, J = 5.8 Hz, 1H), 2.63 (d, J = 4.6 Hz, 3H), |
| | | | | 2.13 (s, 1.5H), 2.12 (s, 1.5H), 2.08-2.02 |
| | | | | (overlapping m, 2H), 1.92-1.78 (m, 2H). |
| 26 | ** | *** | 421.3 | 1H NMR (400 MHz, DMSO-d6) δ 13.29 (br s, 1H), |
| | | | | 9.03 (s, 1H), 8.49 (d, J = 5.7 Hz, 1H), 8.22 (br s, 1H), |
| | | | | 7.95 (br s, 1H), 7.52 (d, J = 5.7 Hz, 1H), 7.27 (ddd, |
| | | | | J = 11.5, 8.9, 2.8 Hz, 1H), 7.05-6.93 (m, 2H), 4.65 (p, |
| | | | | J = 6.9 Hz, 1H), 4.06 (d, J = 11.2 Hz, 4H), 2.76-2.65 |
| | | | | (m, 2H), 2.31-2.14 (m, 2H) |
| 27 | ** | *** | 464.2 | 1H NMR (400 MHz, DMSO-d6): 8.37 (bs, 1H), 8.30 |
| | | | | (s, 1H), 7.90-7.87 (m, 2H), 7.82 (bs, 1H), 7.57 (d, |
| | | | | J = 2.4 Hz, 1H), 7.49 (d, J = 8.8 Hz, 1H), 7.39 (dd, J = 2.4 |
| | | | | Hz, J = 2.8 1H), 3.63 (s, 2H), 3.42 (bs, 6H), 2.61 (bs, 2H). |
| 28 | * | *** | 468.2 | 1H NMR (400 MHz, CDCl3): 9.20 (s, 1H), 8.59 (d, |
| | | | | J = 5.6 Hz, 1H), 8.07 (s, 1H), 7.82 (s, 1H), 7.70 (s, 1H), |
| | | | | 7.69 (dd, J = 0.8 Hz, J = 0.8 Hz, 1H), 7.47-7.43 (m, 1H), |
| | | | | 7.13 (dd, J = 2.4 Hz, 2.8 Hz), 6.99-6.95 (m, 1H), 3.64 |
| | | | | (s, 2H), 3.41-3.39 (bs, 4H), 3.30 (s, 3H), 2.66-2.63 |
| | | | | (bs, 4H). |
| 29 | ** | *** | 509.4 | 1H NMR (400 MHz, DMSO-d6) δ 9.11 (s, 1H), 8.56 |
| | | | | (d, J = 5.8 Hz, 1H), 7.93 (dd, J = 8.3, 7.4 Hz, 1H), |
| | | | | 7.79-7.66 (m, 3H), 7.60-7.51 (m, 2H), 6.95 (d, J = |
| | | | | 7.9 Hz, 1H), 3.84 (s, 3H), 3.64 (s, 2H), 3.44-3.36 |
| | | | | (m, 4H), 3.26 (s, 3H), 2.45-2.39 (m, 4H). |
| 30 | ** | ** | 536.2 | 1H NMR (400 MHz, DMSO-d6) δ 7.87-7.78 (m, |
| | | | | 1H), 7.35 (dd, J = 7.3, 2.2 Hz, 1H), 7.25 (ddd, J = |
| | | | | 14.9, 9.2, 4.2 Hz, 2H), 7.03-6.92 (m, 1H), 6.77 (d, |
| | | | | J = 8.2 Hz, 1H), 5.17-5.07 (m, 1H), 4.61 (d, J = 18.9 |
| | | | | Hz, 2H), 4.48 (bs, 1H), 3.85-3.74 (m, 2H), 3.50-3.44 |
| | | | | (brs, 2H), 3.09-2.96 (m, 2H), 2.93 (d, J = 5.8 Hz, |
| | | | | 1H), 2.80 (d, J = 5.6 Hz, 1H), 2.41-2.31 (m, 3H), |
| | | | | 2.20-2.12 (m, 5H), 1.82 (bs, 2H), 1.78-1.69 (m, 1H), |
| | | | | 1.67-1.45 (m, 3H). |
| 31 | ** | ** | 495.1 | 1H NMR (400 MHz, DMSO-d6) δ 12.78 (s, 0.7H), |
| | | | | 12.63 (s, 0.5H), 8.19-8.13 (bs, 0.5H), 7.91 (bs, 0.6H), |
| | | | | 7.52 (dd, J = 20.1, 9.1 Hz, 1H), 7.35-7.19 (m, 3H), |
| | | | | 7.10-6.93 (m, 1H), 4.58 (d, J = 14.4 Hz, 2H), 4.46 |
| | | | | (bs, 1H), 3.79 (dd, J = 11.9, 6.0 Hz, 2H), 3.53 (bs, |
| | | | | 1H), 2.99-2.90 (m, 3H), 2.76 (t, J = 5.7 Hz, 1H), 2.17- |
| | | | | 2.07 (m, 3H), 1.93-1.90 (bs, 2H), 1.68-1.60 (bs, |
| | | | | 2H), 1.24 (bs, 6H) |
| 32 | *** | ** | 455.4 | 1H NMR (400 MHz, DMSO-d6) δ 13.07 (br s, 1H), |
| | | | | 8.22 (br s, 2H), 7.34-7.24 (m, 2H), 7.05-6.97 (m, |
| | | | | 2H), 4.63 (d, J = 16.9 Hz, 2H), 4.55-4.45 (m, 1H), |
| | | | | 3.83-3.74 (m, 2H), 3.33-3.26 (submerged m, 2H), |
| | | | | 3.00-2.90 (overlapping m, 2H), 2.93-2.87 |
| | | | | (overlapping m, 1H), 2.77 (t, J = 5.7 Hz, 1H), 2.13 (s, |
| | | | | 1.5H), 2.12 (s, 1.5H), 2.10-1.98 (m, 2H), 1.86-1.72 |
| | | | | (m, 2H). |
| 33 | *** | ** | 482.27 | 1H NMR (400 MHz, DMSO-d6) δ 11.24 (submerged |
| | | | | br s, 1H), 7.62-7.53 (m, 1H), 7.33-7.24 (m, 2H), |
| | | | | 7.05-6.95 (m, 1H), 6.84-6.75 (m, 1H), 6.44-6.36 |
| | | | | (m, 1H), 4.65 (d, J = 17.0 Hz, 2H), 4.57-4.45 (m, |
| | | | | 1H), 3.80 (q, J = 5.3 Hz, 2H), 3.45-3.35 (overlapping |
| | | | | br m, 2H), 3.09-2.99 (m, 2H), 2.95 (t, J = 5.7 Hz, |
| | | | | 1H), 2.81 (t, J = 5.5 Hz, 1H), 2.13 (s, 1.5H), 2.11 (s, |
| | | | | 1.5H), 2.03-1.94 (m, 2H), 1.80-1.65 (m, 2H). |
| 34 | ** | ** | 467.4 | 1H NMR (400 MHz, DMSO-d6) δ 9.29 (s, 2H), 9.22 |
| | | | | (d, J = 2.3 Hz, 1H), 7.31-7.23 (m, 2H), 7.03-6.94 |
| | | | | (m, 1H), 4.68 (d, J = 18.2 Hz, 2H), 4.55-4.42 (m, |
| | | | | 1H), 3.87-3.77 (m, 2H), 3.54-3.15 (submerged m, |
| | | | | 2H), 3.06-2.92 (overlapping m, 3H), 2.83 (t, J = 5.7 |
| | | | | Hz, 1H), 2.14 (s, 1.5H), 2.11 (s, 1.5H), 1.93 (dt, J = |
| | | | | 7.8, 5.6 Hz, 2H), 1.71-1.56 (m, 2H). |
| 35 | ** | ** | 408.3 | 1H NMR (400 MHz, DMSO-d6) δ 9.11 (d, J = 0.6 |
| | | | | Hz, 1H), 8.55 (d, J = 5.7 Hz, 1H), 8.46-8.30 (m, 2H), |
| | | | | 7.61 (dd, J = 5.7, 0.6 Hz, 1H), 7.17-6.99 (m, 1H), |
| | | | | 6.94-6.77 (m, 2H), 5.07(J = 8.7, 1.7 Hz, 1H), 3.86 |
| | | | | (d, J = 13.0 Hz, 2H), 3.63-3.44 (m, 1H), 3.01 (t, J = |
| | | | | 11.6 Hz, 2H), 2.02-1.88 (m, 2H), 1.79-1.54 (m, |
| | | | | 2H) ppm. |
| 36 | ** | *** | 496.37 | 1H NMR (400 MHz, DMSO-d6) δ 7.56-7.46 (m, |
| | | | | 1H), 7.32-7.21 (m, 2H), 6.99 (ddd, J = 11.1, 3.8, 2.3 |
| | | | | Hz, 1H), 6.48 (d, J = 9.0 Hz, 1H), 6.40-6.31 (m, |
| | | | | 1H), 4.63 (d, J = 18.1 Hz, 2H), 4.49 (dd, J = 7.6, 3.9 |
| | | | | Hz, 1H), 3.81 (d, J = 3.7 Hz, 2H), 3.51-3.40 (m, |
| | | | | 2H), 3.29 (s, 1.5H), 3.27 (s, 1.5), 3.11-3.00 (m, 2H), |
| | | | | 2.95 (t, J = 5.8 Hz, 1H), 2.86-2.77 (m, 1H), 2.13 (s, |
| | | | | 1.5H), 2.10 (s, 1.5H), 1.93-1.83 (m, 2H), 1.63-1.47 |
| | | | | (m, 2H). |
| 37 | ** | *** | 467.5 | 1H NMR (400 MHz, DMSO-d6) δ 8.96 (d, J = 4.9 |
| | | | | Hz, 2H), 7.54 (t, J = 4.9 Hz, 1H), 7.35-7.17 (m, 2H), |
| | | | | 7.05-6.90 (m, 1H), 4.61 (d, J = 22.0 Hz, 2H), 4.48- |
| | | | | 4.43 (m, 1H), 3.83-3.79 (m, 2H), 3.40-3.37 (m, 2H), |
| | | | | 3.07-2.89 (m, 2H), 2.94 (t, J = 6.2 Hz, 1H), 2.81 (t, |
| | | | | J = 5.9 Hz, 1H), 2.14 (s, 1.5H), 2.10 (s, 1.5H), 1.84- |
| | | | | 1.82 (m, 2H), 1.55-1.48 (m, 2H). |
| 38 | *** | ** | 496.4 | 1H NMR (400 MHz, DMSO-d6) δ 8.68 (d, J = 1.6 |
| | | | | Hz, 1H), 8.33 (t, J = 2.4 Hz, 1H), 7.84-7.78 (m, J = |
| | | | | 2.7, 1.8 Hz, 1H), 7.34-7.20 (m, 2H), 7.04-6.92 (m, |
| | | | | 1H), 4.66 (d, J = 18.9 Hz, 2H), 4.54-4.40 (m, J = |
| | | | | 7.6, 3.9 Hz, 1H), 3.89 (s, 3H), 3.83-3.77 (m, 2H), |
| | | | | 3.33-3.29 (m, 2H), 3.03-2.95 (m, 2H, |
| | | | | overlapping), 2.95 (t, 1H, overlapping), 2.81 (t, J = |
| | | | | 5.7 Hz, 1H), 2.14 (s, 1.5H), 2.11 (s, 1.5H), 1.95- |
| | | | | 1.86 (m, 2H), 1.71-1.52 (m, 2H). |
| 39 | ** | *** | 524.2 | 1H NMR (400 MHz, DMSO-d6) δ 7.80 (dd, J = 11.1, |
| | | | | 4.5 Hz, 1H), 7.36-7.19 (m, 3H), 7.03-6.93 (m, |
| | | | | 1H), 6.74 (d, J = 8.2 Hz, 1H), 5.34-5.24 (m, 1H), |
| | | | | 4.61 (d, J = 19.3 Hz, 2H), 4.52-4.41 (m, 1H), 3.85- |
| | | | | 3.75 (m, 2H), 3.86-3.75 (m, 2H), 3.43 (d, J = 12.5 |
| | | | | Hz, 2H), 2.99 (dd, J = 12.6, 9.7 Hz, 2H), 2.92 (t, J = |
| | | | | 5.6 Hz, 1H), 2.79 (t, J = 5.6 Hz, 1H), 2.12 (d, J = 11.1 |
| | | | | Hz, 3H), 1.83 (bs, 2H), 1.60-1.48 (m, 2H), 1.29 (d, |
| | | | | J = 6.2 Hz, 5H). |
| 40 | ** | ** | 455.27 | 1H NMR (400 MHz, DMSO-d6) δ12.40 (s, 1H), 7.29- |
| | | | | 7.21 (m, 2H), 7.20 (s, 1H), 7.06 (s, 1H), 7.00-6.92 |
| | | | | (m, 1H), 4.62 (s, 1H), 4.60 (s, 1H), 4.48 (dt, J = 11.5, |
| | | | | 3.9 Hz, 1H), 3.77 (overlapping q, J = 5.6 Hz, 2H), |
| | | | | 3.52-3.42 (m, 2H), 3.02 (t, J = 9.8 Hz, 2H), 2.88 (t, |
| | | | | J = 5.7 Hz, 1H), 2.75 (t, J = 5.7 Hz, 1H), 2.10 (s, 2H), |
| | | | | 2.08 (s, 1H), 2.01-1.90(m, 2H), 1.72 (dd, J = 17.4, 8.5 |
| | | | | Hz, 2H). Rotamers observed |
| 41 | ** | ** | 409.2 | 1H NMR (400 MHz, DMSO-d6) δ 13.04 (s, 1H), |
| | | | | 12.51 (s, 1H), 9.09 (s, 1H), 8.54 (s, 1H), 7.88 (d, J = |
| | | | | 6.9 Hz, 1H), 7.57 (s, 1H), 7.33-7.26 (m, 1H), 7.01 |
| | | | | (t, J = 8.7 Hz, 1H), 4.58 (s, 1H), 3.70 (s, 2H), 2.05 (s, |
| | | | | 2H), 1.82 (t, J = 31.0 Hz, 2H). |
| 42 | ** | *** | 424.3 | 1H NMR (400 MHz, CDCl3): 10.58 (s, 1H), 9.24 (s, |
| | | | | 1H), 8.63 (d, J = 5.6 Hz, 1H), 8.17 (s, 1H), 7.86 (s, |
| | | | | 1H), 7.70 (d, J = 5.2 Hz, 1H), 7.52-7.43 (m, 1H), 7.22- |
| | | | | 7.20 (m, 1H), 7.00 (t, J = 2.0 Hz, 1H), 3.67 (s, 2H), |
| | | | | 3.46 (bs, 4H), 2.75 (bs, 4H). |
| 43 | *** | ** | 469.3 | 1H NMR (400 MHz, DMSO-d6) δ 12.69 (br s, 1H), |
| | | | | 8.12-7.95 (m, 1H), 7.41-7.17 (m, 2H), 7.12-6.90 |
| | | | | (m, 1H), 4.60 (d, J = 16.6 Hz, 2H), 4.54-4.34 (m, |
| | | | | 1H), 3.78 (q, J = 6.0 Hz, 2H), 3.34 (submerged m, |
| | | | | 2H), 2.99-2.85 (m, 3H), 2.75 (t, J = 5.8 Hz, 1H), |
| | | | | 2.40 (d, J = 1.3 Hz, 3H), 2.12 (s, 1.5H), 2.11 (s, |
| | | | | 1.5H), 2.03-1.91 (m, 2H), 1.75-1.63 (m, 2H). |
| 44 | *** | ** | 469.4 | 1H NMR (400 MHz, DMSO-d6) δ 8.32 (s, 1H), 8.05 |
| | | | | (s, 1H), 7.34-7.23 (m, 2H), 7.01 (ddd, J = 9.1, 2.6, |
| | | | | 1.4 Hz, 1H), 4.62 (d, J = 15.7 Hz, 2H), 4.56-4.45 |
| | | | | (m, 1H), 3.92 (s, 3H), 3.79 (dd, J = 9.3, 5.0 Hz, 2H), |
| | | | | 2.96 (t, J = 10.0 Hz, 2H), 2.90 (t, J = 5.3 Hz, 1H), |
| | | | | 2.76 (t, J = 5.5 Hz, 1H), 2.12 (s, 1.5H), 2.11 (s, 1.5H), |
| | | | | 2.10-2.00 (m, 2H), 1.90-1.77 (m, 2H). |
| 45 | ** | ** | 422.4 | 1H NMR (400 MHz, DMSO-d6) 5:1 mixture of |
| | | | | tropodiastereoisomers (only major is described) 8 |
| | | | | 13.30 (bs, 1H, pyrazole NH), 9.13 (d, J = 0.6 Hz, 1H), |
| | | | | 8.55 (d, J = 5.7 Hz, 1H), 8.45 (bs, 1H), 8.22-8.12 |
| | | | | (overlapping, m, 1H), 7.62 (dd, J = 5.7, 0.6 Hz, 1H), |
| | | | | 7.53-7.43 (m, 1H), 7.25-7.18 (m, 1H), 7.08 (td, J = |
| | | | | 8.4, 2.2 Hz, 1H), 4.22-4.10 (m, 1H), 3.60-3.47 (m, |
| | | | | 2H), 3.37-3.34 (overlapping, m, 4H), 2.76-2.65 |
| | | | | (m, 1H) 2.39-2.29 (m, 1H), 1.10 (d, J = 6.5 Hz, 3H). |
| 46 | *** | ** | 454.3 | 1H NMR (400 MHz, DMSO-d6): δ 11.06 (s, 1H), |
| | | | | 7.55 (s, 1H), 7.36-7.23 (m, 2H), 7.01 (s, 1H), 6.82 |
| | | | | (s, 1H), 6.75 (s, 1H), 4.60 (d, J = 16.5 Hz, 2H), 4.50 |
| | | | | (s, 1H), 3.78 (d, J = 4.9 Hz, 2H), 3.38 (d, J = 9.3 Hz, |
| | | | | 2H), 2.92 (dd, J = 22.5, 12.7 Hz, 3H), 2.74 (s, 1H), |
| | | | | 2.12 (d, J = 3.2 Hz, 3H), 2.05 (d, J = 16.3 Hz, 2H), |
| | | | | 1.78 (d, J = 9.1 Hz, 2H). |
| 47 | ** | ** | 567.3 | 1H NMR (400 MHz, DMSO-d6) δ 7.32-7.19 (m, |
| | | | | 2H), 7.04-6.93 (m, 1H), 5.38 (d, J = 7.3 Hz, 1H), |
| | | | | 4.60 (d, J = 17.1 Hz, 1H), 4.43 (m, 1H), 3.87 (d, J = |
| | | | | 10.4 Hz, 1H), 3.83-3.76 (m, 2H), 3.69-3.58 (m, |
| | | | | 1H), 2.92 (bs, 3H), 2.79 (bs, 1H), 2.32-2.25 (m, |
| | | | | 1H), 2.11 (dd, J = 7.5, 3.4 Hz, 3H), 2.01-1.99 (m, |
| | | | | 1H), 1.86 (d, J = 10.6 Hz, 2H), 1.69 (bs, 1H), 1.54 |
| | | | | (bs, 4H). |
| 48 | ** | ** | 509.3 | 1H NMR (400 MHz, DMSO-d6): δ 7.71 (s, 1H), 7.25 |
| | | | | (qd, J = 11.6, 4.5 Hz, 2H), 7.11-6.92 (m, 2H), 6.77 |
| | | | | (s, 1H), 4.62 (d, J = 14.5 Hz, 2H), 4.47 (s, 1H), 3.77 |
| | | | | (dd, J = 23.8, 17.8 Hz, 2H), 3.46 (d, J = 12.4 Hz, 2H), |
| | | | | 3.10 (d, J = 5.9 Hz, 6H), 3.06-2.97 (m, 2H), 2.93 (s, |
| | | | | 1H), 2.79 (s, 1H), 2.20-2.06 (m, 3H), 1.88 (d, J = |
| | | | | 20.8 Hz, 2H), 1.58 (d, J = 8.6 Hz, 2H). |
| 49 | ** | ** | 422.4 | 1H NMR (400 MHz, DMSO-d6) δ 13.32 (s, 1H, |
| | | | | pyrazole NH), 9.11 (s, 1H), 8.55 (d, J = 5.7 Hz, 1H), |
| | | | | 8.44 (s, 1H), 8.18 (s, 1H), 7.62 (d, J = 5.7 Hz, 1H), |
| | | | | 7.48 (dd, J = 15.6, 8.4 Hz, 1H), 7.20 (td, J = 10.3, 2.3 |
| | | | | Hz, 1H), 7.11-6.97 (m, 1H), 3.98 (d, J = 13.7 Hz, |
| | | | | 1H), 3.73-3.55 (m, 2H), 3.28 (d, J = 13.7 Hz, 1H, |
| | | | | overlapping), 2.99 (t, J = 10.7 Hz, 1H), 2.88-2.77 |
| | | | | (m, 1H), 2.71-2.59 (m, 2H), 2.37-2.21 (m, 1H), |
| | | | | 1.10 (d, J = 6.0 Hz, 3H). |
| 50 | *** | ** | 534.5 | 1H NMR (400 MHz, DMSO-d6) δ 8.28-8.19 (m, |
| | | | | 1H), 8.19-8.12 (m, 1H), 7.96-7.88 (m, 1H), 7.35- |
| | | | | 7.18 (m, 2H), 7.02-6.91 (m, 1H), 4.74-4.59 (m, |
| | | | | 2H), 4.51-4.41 (m, 1H), 3.86-3.74 (m, 2H), 3.45- |
| | | | | 3.36 (m, 2H), 3.06-2.90 (m, 2H, overlapped), 2.95 |
| | | | | (t, 2H, overlapped), 2.86-2.75 (m, 1H), 2.14 (s, |
| | | | | 1.5H), 2.11 (s, 1H), 1.92-1.76 (m, 2H), 1.65-1.43 |
| | | | | (m, 2H). |
| 51 | *** | ** | 482.4 | 1H NMR (400 MHz, DMSO-d6) δ 8.38 (br s, 1H), |
| | | | | 8.11 (d, J = 2.1 Hz, 1H), 8.08 (dd, J = 9.5, 2.6 Hz, |
| | | | | 1H), 7.35-7.24 (m, 2H), 7.06-6.96 (m, 1H), 6.45 |
| | | | | (dd, J = 9.5, 3.1 Hz, 1H), 4.62 (d, J = 18.9 Hz, 2H), |
| | | | | 4.54-4.43 (m, 1H), 3.86-3.70 (m, 2H), 3.39 |
| | | | | (overlapping m, 2H), 3.01-2.93 (m, 2H), 2.90 (t, J = |
| | | | | 5.8 Hz, 1H), 2.77 (t, J = 6.0 Hz, 1H), 2.13 (s, 1.5H), |
| | | | | 2.10 (s, 1.5H), 2.06-1.94 (m, 2H), 1.81-1.66 (m, 2H). |
| 52 | *** | ** | 408.4 | 1H NMR (400 MHz, DMSO-d6) δ 13.31 (bs, 1H), |
| | | | | 9.12 (s, 1H), 8.55 (d, J = 5.7 Hz, 1H), 8.47 (s, 1H), |
| | | | | 8.19 (s, 1H), 7.62 (d, J = 5.7 Hz, 1H), 7.47 (dd, J = |
| | | | | 15.5, 8.4 Hz, 1H), 7.21 (td, J = 10.2, 2.3 Hz 1H), 7.07 |
| | | | | (td, J = 8.4, 1.9 Hz, 1H), 3.57 (s, 2H), 3.43-3.34 (m, |
| | | | | 4H, overlapping), 2.62-2.53 (m, 4H, overlapping). |
| 53 | ** | *** | 554.2 | 1H NMR (400 MHz, DMSO-d6) δ 8.04 (s, 1H), 7.38- |
| | | | | 7.24 (m, 3H), 7.06-6.97 (m, 1H), 6.93 (dd, J = 3.3, |
| | | | | 1.5 Hz, 1H), 4.64 (d, J = 18.6 Hz, 2H), 4.54 (bs, 1H), |
| | | | | 3.69-3.79 (m, 2H), 2.94-2.90 (m, 3H), 2.78 (t, J = 5.7 |
| | | | | Hz, 1H), 2.10-2.04 (m, 5H), 1.89-1.79 (m, 2H), 1.60 |
| | | | | (s, 9H). |
| 54 | ** | ** | 409.2 | 1H NMR (400 MHz, DMSO-d6) δ 9.27(s, 1H), 8.91 |
| | | | | (br s, 1H), 8.65 (d, J = 6 Hz, 1H), 8.30 (s, 1H), ), 7.77 |
| | | | | (d, J = 6 Hz, 1H), 7.35-7.27 (m, 2H), 7.05-6.99 |
| | | | | (m, 1H), 4.62-4.60 (m, 1H), 3.78-3.74 (m, 2H), 3.53 |
| | | | | (t, J = 10 Hz, , J = 9.6 Hz, 2H), 2.08-2.05 (m, 2H), |
| | | | | 1.85-1.80 (m, 2H). |
| 55 | ** | ** | 422.3 | 1H NMR (400 MHz, DMSO-d6) δ 13.3 (submerged |
| | | | | brs, 1H), 9.11 (d, J = 0.4 Hz, 1H), 8.54 (d, J = 5.7 Hz, |
| | | | | 1H), 8.48-8.17 (m, 2 H), 7.60 (d, J = 5.7 Hz, 1H), |
| | | | | 7.20-7.12 (m, 2H), 7.01-6.93 (m, 1H), 3.89 (d, J = |
| | | | | 13.1 Hz, 1H), 3.28-3.20 (overlapping m, 1H), 2.88 (t, |
| | | | | J = 11.3 Hz, 2H), 2.68 (s, 3H), 1.90-1.66 (m, 4H). |
| 56 | ** | *** | 497.5 | 1H NMR (400 MHz, DMSO-d6) δ 8.62 (s, 1H), 7.32- |
| | | | | 7.14 (m, 2H), 7.01-6.86 (m, 2H), 4.55 (d, J = 26.0 |
| | | | | Hz, 2H), 4.50-4.36 (m, 1H), 3.91 (s, 3H), 3.84-3.67 |
| | | | | (m, 2H), 3.58-3.35 (m, 2H), 3.09-2.94 (m, 2H), 2.94- |
| | | | | 2.83 (m, 1H), 2.82-2.71 (m, 1H), 2.08 (s, 1.5H), |
| | | | | 2.05 (s, 1.5H), 1.92-1.73 (m, 2H), 1.67-1.41 (m, 2H). |
| 57 | ** | ** | 484.2 | 1H NMR (400 MHz, DMSO-d6) δ = 8.22-8.04 (m, |
| | | | | 1H), 7.85 (dd, J = 2.4, 7.3 Hz, 1H), 7.31-7.18 (m, |
| | | | | 3H), 7.02-6.95 (m, 1H), 4.69-4.59 (m, 2H), 4.52- |
| | | | | 4.45 (m, 1H), 3.92-3.68 (m, 9H), 3.48-3.43 (m, |
| | | | | 2H), 3.02 (br t, J = 10.9 Hz, 2H), 2.94 (br t, J = 5.7 |
| | | | | Hz, 2H), 2.81 (br t, J = 5.7 Hz, 1H), 2.12 (d, J = 11.0 |
| | | | | Hz, 3H), 1.95-1.87 (m, 2H), 1.68-1.56 (m, 2H) |
| 58 | ** | ** | 442.24 | 1H NMR (400 MHz, DMSO-d6) δ 13.35 (Submerged |
| | | | | brs, 1H), 9.18 (s, 1H), 8.60 (d, J = 5.7 Hz, 1H), 8.44 |
| | | | | (brs, 2H), 7.69 (d, J = 5.7 Hz, 1H), 7.22-7.11 |
| | | | | (m, 2H), 6.96-6.87 (m, 1H), 4.06-3.93 (m, 2H), |
| | | | | 3.82-3.68 (m, 2H), 3.64-3.47 (m, 4H). |
| 59 | ** | ** | 532.4 | 1H NMR (400 MHz, DMSO-d6) δ 7.84 (t, J = 7.8 Hz, |
| | | | | 1H), 7.39 (d, J = 7.3 Hz, 1H), 7.30-7.21 (m, 2H), |
| | | | | 7.01-6.94 (m, 1H), 6.83 (d, J = 8.3 Hz, 1H), 4.52- |
| | | | | 4.43 (m, 1H), 4.36 (s, 2H), 3.88 (s, 3H), 3.56 (t, J = |
| | | | | 5.9 Hz, 2H), 3.50-3.40 (m, 2H), 3.07-2.99 |
| | | | | (overlapping multiplet, 5H), 2.95 (t, J = 5.8 Hz, 2H), |
| | | | | 1.92-1.79 (m, 2H), 1.64-1.46 (m, 2H). |
| 60 | *** | ** | 532.5 | 1H NMR (400 MHz, DMSO-d6) δ 8.08-8.04 (m, 1H), |
| | | | | 7.83-7.46 (t, J = 72.7 Hz, 1H, CHF2), 7.69 (dd, J = |
| | | | | 7.3, 2.7 Hz, 1H), 7.32-7.19 (m, 2H), 7.11 (dd, J = |
| | | | | 8.0, 2.7 Hz, 1H), 7.04-6.88 (m, 1H), 4.62 (d, J = |
| | | | | 19.1 Hz, 2H), 4.54-4.41 (m, 1H), 3.87-3.71 (m, J = |
| | | | | 9.9, 5.6 Hz, 2H), 3.41-3.40 (m, 2H), 3.10-2.96 (m, |
| | | | | 2H), 2.93 (t, J = 5.8 Hz, 1H), 2.80 (t, J = 5.7 Hz, 1H), |
| | | | | 2.13 (s, 1.5H), 2.10 (s, 1.5H), 1.95-1.78 (m, 2H), |
| | | | | 1.72-1.43 (m, 2H). |
| 61 | ** | ** | 552.2 | 1H NMR (400 MHz, DMSO-d6) δ 7.84 (dd, J = 11.1, |
| | | | | 4.5 Hz, 1H), 7.38 (dd, J = 7.3, 2.1 Hz, 1H), 7.31- |
| | | | | 7.21 (m, 2H), 7.02-6.93 (m, 1H), 6.87-6.79 (m, |
| | | | | 1H), 5.52 (bs, 1H), 4.61 (d, J = 18.6 Hz, 2H), 4.49 (m, |
| | | | | 1H), 3.94-3.72 (m, 6H), 3.50-3.43 (m, 2H), 3.08- |
| | | | | 2.97 (m, 2H), 2.92 (t, J = 5.7 Hz, 1H), 2.79 (t, J = 5.4 |
| | | | | Hz, 1H), 2.22-2.13 (m, 1H), 2.12 (d, J = 11.0 Hz, 3H), |
| | | | | 2.06-1.95 (m, 1H), 1.84 (bs, 2H), 1.54-1.50 (m, 2H). |
| 62 | ** | ** | 495.1 | 1H NMR (400 MHz, DMSO-d6): 7.98 (d, J = 7.9 Hz, |
| | | | | 1H), 7.27 (ddd, J = 11.8, 7.3, 3.1 Hz, 2H), 7.03-6.94 |
| | | | | (m, 1H), 4.60 (d, J = 17.3 Hz, 2H), 4.46 (s, 1H), 3.79 |
| | | | | (dd, J = 11.7, 5.7 Hz, 4H), 3.35 (s, 3H), 3.10 (ddd, J = |
| | | | | 14.5, 7.3, 4.8 Hz, 1H), 2.92 (dd, J = 16.6, 7.9 Hz, 3H), |
| | | | | 2.76 (t, J = 5.9 Hz, 1H), 2.43-2.35 (m, 1H), 2.14- |
| | | | | 2.09 (m, 3H), 1.92 (d, J = 12.3 Hz, 1H), 1.68 (d, J = |
| | | | | 8.9 Hz, 2H), 1.18 (t, J = 7.3 Hz, 1H), 0.88 (d, J = 8.3 |
| | | | | Hz, 2H), 0.81 (dd, J = 5.1, 2.7 Hz, 2H). |
| 63 | ** | *** | 453.4 | 1H NMR (400 MHz, DMSO-d6) δ 13.23 (bs, 1H), |
| | | | | 9.11 (s, 1H), 8.55 (d, J = 5.7 Hz, 1H), 8.36 (s, 1H), |
| | | | | 8.16 (s, 1H), 7.62 (dd, J = 5.7, 0.4 Hz, 1H), 7.36- |
| | | | | 7.21 (m, 2H), 7.05-6.93 (m, 1H), 3.60 (dd, J = 8.8, |
| | | | | 4.6 Hz, 2H), 3.45 (overlapping s, 2H), 3.43-3.37 |
| | | | | (overlapping m, 2H), 3.22 (s, 3H), 1.97-1.83 (m, 4H). |
| 64 | *** | *** | 440.2 | 1H NMR (400 MHz, CDCl3): 9.24 (s, 1H), 8.64 (d, |
| | | | | J = 5.6 Hz, 1H), 8.17 (s, 1H), 7.88(s, 1H), 7.74 (d, J = |
| | | | | 5.6 Hz, 1H), 7.64 (bs, 1H), 7.32 (d, J = 8.8 Hz, 1H), |
| | | | | 3.79 (s, 2H), 3.54 (bs, 4H), 2.86 (bs, 4H). |
| 65 | ** | *** | 422.1 | 1H NMR (400 MHz, CDCl3): 10.63-10.54 (bs, 1H), |
| | | | | 9.23 (s, 1H), 8.63 (d, J = 5.6 Hz, 1H), 8.19 (s, 1H), |
| | | | | 7.87 (s, 1H), 7.70 (s, 1H), 7.52-7.47 (m, 1H), 7.14 |
| | | | | (dd, J = 2.4 Hz, J = 2.8 Hz, 1H), 7.01-6.96 (m, 1H), 3.70 |
| | | | | (s, 2H), 3.49 (bs, 4H), 2.76 (bs, 4H). |
| 66 | ** | ** | 482.4 | 1H NMR (400 MHz, DMSO-d6) δ 8.37 (br s, 1H), |
| | | | | 7.47 (dd, J = 6.8, 2.5 Hz, 1H), 7.36-7.23 (m, 2H), |
| | | | | 7.06-6.94 (m, 1H), 6.78 (s, 1H), 6.63 (d, J = 6.3 Hz, |
| | | | | 1H), 4.63 (d, J = 20.0 Hz, 2H), 4.56-4.45 (m, 1H), |
| | | | | 3.83-3.77 (m, 2H), 3.46 (overlapping m, 2H), 3.09- |
| | | | | 3.00 (m, 2H), 2.93 (t, J = 5.6 Hz, 1H), 2.79 (t, J = 5.6 |
| | | | | Hz, 1H), 2.13 (s, 1.5H), 2.11 (s, 1.5H), 2.02-1.92 |
| | | | | (m, 2H), 1.73-1.61 (m, 2H). |
| 67 | ** | ** | 499.3 | 1H NMR (400 MHz, CDCl3): 7.72 (s, 1.5H), 7.67 (s, |
| | | | | 0.5 H), 6.99-6.90 (m, 1H), 6.89-6.80 (m, 1H), 6.79- |
| | | | | 6.75 (m, 1H), 5.69 (d, J = 3.9 Hz, 2H), 4.76 (s, 1H), |
| | | | | 4.62 (s, 1H), 4.35-4.32 (m, 1H), 3.96 (t, J = 6.0 Hz, |
| | | | | 1H), 3.80 (t, J = 6.0 Hz, 1H), 3.48-3.47 (m, 1H), 3.22 |
| | | | | (d, J = 4.8 Hz), 3.09-3.03 (m, 2H), 2.97 (t, J = 2.9 Hz |
| | | | | 1H), 2.94 (t, J = 3.0 Hz 1H), 2.22 (d, J = 2.7 Hz, 3H), |
| | | | | 2.00-1.95 (m, 2H), 1.88-1.83 (m, 2H). |
| 68 | *** | ** | 480.5 | 1H NMR (400 MHz, DMSO-d6) δ 7.80 (t, J = 7.7 Hz, |
| | | | | 1H), 7.60 (d, J = 7.3 Hz, 1H), 7.31-7.17 (m, 3H), |
| | | | | 7.02-6.91 (m, 1H), 4.62 (d, J = 21.0 Hz, 2H), 4.45 |
| | | | | (tt, J = 9.2, 4.4 Hz, 1H), 3.80 (q, J = 5.7 Hz, 2H), |
| | | | | 3.45-3.35 (m, 2H), 3.02-2.94 (m, 2H), 2.92 (t, J = |
| | | | | 5.9 Hz, 1H), 2.79 (t, J = 5.7 Hz, 1H), 2.51 (s, 3H), |
| | | | | 2.13 (s, 1.5H), 2.10 (s, 1.5H), 1.90-1.80 (m, 2H), 1.66- |
| | | | | 1.48 (m, 2H). |
| 69 | ** | ** | 512.5 | 1H NMR (400 MHz, DMSO-d6) δ 7.35-7.24 (m, |
| | | | | 2H), 7.05-6.98 (m, 1H), 6.48-6-41 (m, 1H), 4.57 (d, |
| | | | | J = 17.5 Hz, 2H), 4.53-4.46 (m, 1H), 4.14 (d, J = |
| | | | | 25.0 Hz, 2H), 3.76 (dd, J = 10.2, 5.4 Hz, 2H), 3.68- |
| | | | | 3.58 (m, 2H), 3.57-3.47 (m, 2H), 3.12-3.01 (m, |
| | | | | 2H), 2.87 (t, J = 5.6 Hz, 1H), 2.73 (t, J = 5.4 Hz, 1H), |
| | | | | 2.64-2.56 (overlapped m, 2H), 2.11 (s, 1.7H), 2.09 |
| | | | | (s, 1.3H), 2.07 (s, 1.7H), 2.04 (s, 1.3H), 2.03-1.96 |
| | | | | (m, 2H), 1.76-1.64 (m, 2H). |
| 70 | *** | ** | 491.3 | 1H NMR (400 MHz, DMSO-d6) δ 13.16-13.06 (br, |
| | | | | 1H), 8.35 (s, 1H), 8.11 (s, 1H), 7.38-7.21 (m, 2H), |
| | | | | 7.10-6.93 (m, 1H), 4.59-4.44 (m, 1H), 4.37 (s, 2H), |
| | | | | 3.55 (t, J = 5.9 Hz, 2H), 3.37-3.34 (overlapping |
| | | | | multiplet, 1H, ), 3.32-3.29 (overlapping multiplet, |
| | | | | 1H), 3.03 (s, 3H), 3.00-2.90 (m, 4H), 2.10-2.00 (m, |
| | | | | 2H), 1.89-1.73 (m, 2H). |
| 71 | ** | ** | 512.5 | 1H NMR (400 MHz, DMSO-d6) δ 7.30 (m, 2H), 7.01 |
| | | | | (t, J = 8.5 Hz, 1H), 6.71-6.57 (m, 1H), 4.58 |
| | | | | (overlapped d, J = 19.7 Hz, 2H), 4.55-4.45 |
| | | | | (overlapped m, 1H), 4.37 (s, 2H), 3.76 (s, 2H), 3.65- |
| | | | | 3.44 (m, 4H), 3.12-3.01 (m, 2H), 2.86 (t, J = 5.0 Hz, |
| | | | | 1H), 2.73 (t, J = 4.7 Hz, 1H), 2.37 (s, 1.3H), 2.26 (s, |
| | | | | 0.7H), 2.12 (s, 1.5H), 2.10 (s, 1.5H), 2.07 (s, 1.5H), |
| | | | | 2.06 (s, 1.5H), 2.01 (m, 2H), 1.77-1.64 (m, 2H). |
| 72 | ** | ** | 498.4 | 1H NMR (400 MHz, DMSO-d6) δ 7.35-7.23 (m, |
| | | | | 2H), 7.07-6.90 (m, 1H), 6.87-6.82 (m, 0.5 H), 6.82- |
| | | | | 6.79 (m, 0.5 H), 4.71 (br s, 1H), 4.65-4.57 (m, |
| | | | | 2H), 4.57-4.47 (m, 3H), 4.34 (br s, 1H), 3.86-3.67 |
| | | | | (m, 2H), 3.50-3.37 (m, 2H), 3.11-2.97 (m, 2 H), |
| | | | | 2.96-2.88 (m, 1H), 2.84-2.73 (m, 1H), 2.12 |
| | | | | (overlapping singlet, 1.5H), 2.11-2.09 (overlapping |
| | | | | singlet, 1.5 H), 2.09-2.02 (overlapping multiplet, |
| | | | | 3.5H), 2.00 (overlapping singlet, 1.5H), 1.83-1.72 |
| | | | | (m, 2H). |
| 73 | *** | ** | 508.34 | 1H NMR (400 MHz, DMSO-d6) δ 8.48 (s, 1H), 8.13 |
| | | | | (d, J = 2.3 Hz, 1H), 7.36-7.22 (m, 2H), 7.07-6.92 |
| | | | | (m, 1H), 4.63 (d, J = 16.7 Hz, 2H), 4.56-4.46 (m, |
| | | | | 3H), 3.79 (dd, J = 9.7, 5.6 Hz, 2H), 3.32-3.27 |
| | | | | (overlapping m, 2H), 3.11 (t, J = 6.4 Hz, 2H), 2.94 |
| | | | | (dt, J = 11.5, 7.9 Hz, 3H), 2.77 (t, J = 5.7 Hz, 1H), |
| | | | | 2.13 (s, 1.5H), 2.12 (s, 1.5H), 2.10-2.02 |
| | | | | (overlapping m, 2H), 1.92-1.79 (m, 2H). |
| 74 | *** | ** | 513.4 | 1H NMR (400 MHz, DMSO-d6) δ as a mixture of |
| | | | | isomers 8.36 (s, 1H), 8.05 (s, 0.5H), 8.05 (s, 0.5H), |
| | | | | 7.34-7.24 (m, 2H), 7.04-6.97 (m, 1H), 4.65 (s, |
| | | | | 1H), 4.60 (s, 1H), 4.56-4.46 (m, 1H), 4.35 (t, J = 5.1 |
| | | | | Hz, 2H), 3.82-3.76 (m, 2H), 3.71 (t, J = 5.2 Hz, 2H), |
| | | | | 3.33-3.37 (submerge m, 2H), 3.25 (s, 3H), 2.98- |
| | | | | 2.87 (m, 3H), 2.77 (t, J = 5.8 Hz, 1H), 2.13 (s, 1.5H) |
| | | | | & 2.11 (s, 1.5H), 2.09-2.00 (m, 2H), 1.89-1.78 (m, 2H). |
| 75 | ** | ** | 472.19 | 1H NMR (400 MHz, DMSO-d6) δ 8.00 (t, J = 3.1 Hz, |
| | | | | 1H), 7.84 (t, J = 3.3 Hz, 1H), 7.35-7.25 (m, 2H), |
| | | | | 7.05-6.97 (m, 1H), 4.66 (d, J = 19.9 Hz, 2H), 4.61- |
| | | | | 4.53 (m, 1H), 3.81 (t, J = 5.9 Hz, 2H), 3.64-3.52 (m, |
| | | | | 2H), 3.20-3.08 (m, 2H), 2.95 (t, J = 5.9 Hz, 1H), |
| | | | | 2.81 (t, J = 5.8 Hz, 1H), 2.14 (s, 1.5H), 2.13 (s, 1.5H), |
| | | | | 2.10-2.00 (m, 2H), 1.85-1.73 (m, 2H). |
| 76 | *** | ** | 531.4 | 1H NMR (400 MHz, DMSO-d6) δ 8.96 (s, 1H), 8.37 |
| | | | | (d, J = 3.1 Hz, 1H), 7.94 (d, J = 8.4 Hz, 2H), 7.54 (t, |
| | | | | J = 7.6 Hz, 2H), 7.40-7.22 (m, 3H), 7.05-6.95 (m, |
| | | | | 1H), 4.67 (d, J = 13.3 Hz, 2H), 4.57-4.47 (m, 1H), |
| | | | | 3.87-3.71 (m, 2H), 3.43-3.33 (m, 2H, overlapped), |
| | | | | 3.04 (t, J = 10.3 Hz, 2H), 2.94 (t, J = 5.6 Hz, 1H), |
| | | | | 2.80 (t, J = 5.7 Hz, 1H), 2.14 (s, 1.5H), 2.13 (s, 1.5H), |
| | | | | 2.11-2.02 (m, 2H), 1.91-1.75 (m, 2H). |
| 77 | *** | ** | 511.5 | 1H NMR (400 MHz, DMSO-d6) δ 8.46 (s, 1H), 8.21 |
| | | | | (d, J = 2.2 Hz, 1H), 7.37-7.20 (m, 2H), 7.08-6.93 |
| | | | | (m), 5.87-5.61 (m, 1H), 4.99-4.89 (m, 4H), 4.63 |
| | | | | (d, J = 15.4 Hz, 2H), 4.55-4.47 (m, 1H), 3.79 (dd, |
| | | | | J = 9.1, 5.3 Hz, 2H), 2.97 (t, J = 9.9 Hz, 2H), 2.91 (t, |
| | | | | J = 5.7 Hz, 1H), 2.77 (t, J = 5.7 Hz, 1H), 2.13 (s, 1.5H), |
| | | | | 2.11 (s, 1.5H), 2.10-2.02 (m, 2H), 1.89-1.78 (m, |
| | | | | 2H). 1H submerged under DMSO-d6-water peak. |
| 78 | ** | ** | 496.4 | 1H NMR (400 MHz, DMSO-d6) δ 7.77 (dd, J = 7.0, |
| | | | | 2.4 Hz, 1H), 7.36-7.19 (m, 2H), 7.06-6.93 (m, |
| | | | | 1H), 6.87 (d, J = 1.3 Hz, 1H), 6.68 (dd, J = 7.0, 1.3 |
| | | | | Hz, 1H), 4.63 (d, J = 18.5 Hz, 2H), 4.54-4.44 (m, |
| | | | | 1H), 3.84-3.73 (m, 2H), 3.52-3.40 (overlapping m, |
| | | | | 2H), 3.46 (overlapping s, 3H), 3.03 (t, J = 10.6 Hz, |
| | | | | 2H), 2.93 (t, J = 5.6 Hz, 1H), 2.79 (t, J = 5.6 Hz, 1H), |
| | | | | 2.13 (s, 1.5H), 2.11 (s, 1.5H), 2.02-1.91 (m, 2H), |
| | | | | 1.75-1.60 (m, 2H). |
| 79 | ** | ** | 466.5 | 1H NMR (400 MHz, DMSO-d6) δ 8.71-8.63 (m, |
| | | | | 1H), 7.97-7.88 (m, 1H), 7.81 (d, J = 7.9 Hz, 1H), |
| | | | | 7.46-7.36 (m, 1H), 7.31-7.17 (m, 2H), 7.04-6.90 |
| | | | | (m, 1H), 4.62 (d, J = 19.3 Hz, 2H), 4.50-4.38 (m, |
| | | | | 1H), 3.81 (q, J = 5.7 Hz, 2H), 3.48-3.35 (m, 2H), |
| | | | | 2.98 (m, 2H), 2.93 (t, J = 6.0 Hz, 1H), 2.80 (t, J = 5.8 |
| | | | | Hz, 1H), 2.14 (s, 1.5H), 2.11 (s, 1.5H), 1.96-1.76 |
| | | | | (m, 2H), 1.66-1.47 (m, 2H). |
| 80 | * | ** | 496.5 | 1H NMR (400 MHz, DMSO-d6) δ 8.25 (s, 1H), 7.59 |
| | | | | (d, J = 8.3 Hz, 1H), 7.46 (s, 1H), 7.33-7.14 (m, 2H), |
| | | | | 7.03-6.85 (m, 1H), 4.59 (d, J = 20.7 Hz, 2H), 4.39 |
| | | | | (s, 1H), 3.81 (m, J = 5.2 Hz, 2H, overlapping), 3.78 |
| | | | | (s, 1.5H), 3.77 (s, 1.5H), 3.40-3.27 (m, 2H, |
| | | | | overlapping), 3.00-2.85 (m, 2H), 2.85-2.73 (m, |
| | | | | 2H), 2.13 (s, 1.5H), 2.09 (s, 1.5H), 1.81-1.67 (m, |
| | | | | 2H), 1.50-1.31 (m, J = 8.8 Hz, 2H). |
| 81 | *** | ** | 494.4 | 1H NMR (400 MHz, DMSO-d6) δ 8.53 (d, J = 2.4 |
| | | | | Hz, 1H), 8.20 (d, J = 4.0 Hz, 1H), 7.30 (tt, J = 5.1, 4.6 |
| | | | | Hz, 2H), 7.04-6.98 (m, 1H), 5.61 (d, J = 2.8 Hz, |
| | | | | 2H), 4.64 (d, J = 17.1 Hz, 2H), 4.54-4.49 (m, 1H), |
| | | | | 3.80-3.78 (m, 2H), 3.31-3.25 (overlapping m, 2H), |
| | | | | 2.98-2.88 (m, 3H), 2.78 (t, J = 5.7 Hz, 1H), 2.13 (s, |
| | | | | 1.5H), 2.12 (s, 1.5H), 2.09-2.03 (overlapping m, |
| | | | | 2H), 1.94-1.74 (m, 2H). |
| 82 | ** | ** | 559.5 | 1H NMR (400 MHz, DMSO-d6) δ 8.55 (s, 1H), 8.22 |
| | | | | (d, J = 2.4 Hz, 1H), 7.36-7.24 (m, 2H), 7.06-6.94 |
| | | | | (m, 1H), 5.62-5.51 (m, 1H), 4.86-4.76 (m, 2H), |
| | | | | 4.71 (dd, J = 13.9, 4.9 Hz, 2H), 4.63 (d, J = 15.3 Hz, |
| | | | | 2H), 4.57-4.47 (m, 1H), 3.86-3.73 (m, 2H), 3.34- |
| | | | | 3.26 (overlapping m, 1H), 2.97 (t, J = 10.0 Hz, 2H), |
| | | | | 2.91 (t, J = 5.5 Hz, 1H), 2.78 (t, J = 5.4 Hz, 1H), 2.13 |
| | | | | (s, 1.5H), 2.11 (s, 1.5H), 2.10 2.05 (overlapping m, |
| | | | | 2H), 1.92-1.77 (m, 2H). |
| 83 | * ** | ** | 513.51 | 1H NMR (400 MHz, DMSO-d6) δ 7.83-7.77 (m, |
| | | | | 1H), 7.44 (m, 1H), 7.30-7.22 (m, 2H), 7.01-6.95 |
| | | | | (m, 1H), 4.61 (d, J = 19.8 Hz, 2H), 4.51-4.45 (m, |
| | | | | 1H), 3.97 (s, 3H), 3.82-3.78 (m, 2H), 3.42 (m, 2H), |
| | | | | 3.09-2.99 (m, 2H), 2.92 (t, J = 5.8 Hz, 1H), 2.79 (t, |
| | | | | J = 5.8 Hz, 1H), 2.13 (s, 1.5H), 2.10 (s, 1.5H), 1.90- |
| | | | | 1.88 (m, 2H), 1.60-1.52 (m, 2H). |
| 84 | ** | ** | 455.3 | 1H NMR (400 MHz, DMSO-d6) δ 7.74 (s, 1H), 7.70 |
| | | | | (s, 1H), , 7.38-7.25 (m, 2H), 7.03-6.98 (m, 1H), |
| | | | | 4.66 (d, J = 8.4 Hz, 2H), 4.52 (br s, 1H), 3.80-3.78 |
| | | | | (m, 2H), 3.32 (1H) 1H merged with moisture 3.01 (t, |
| | | | | J = 9.6, J = 9.2 Hz, 2H), 2.93 (t, J = 5.6, J = 5.6 Hz, |
| | | | | 1H), 2.79 (t, J = 5.2, J = 5.6 Hz, 1H), 2.13(d, J = 5.6 |
| | | | | Hz, 3H), 2.07-2.04 (m, 2H), 1.82-1.80 (m, 2H)ppm. |
| 85 | ** | ** | 409.2 | 1H NMR (400 MHz, DMSO-d6) δ 13.31 (s, 1H), 9.19 |
| | | | | (s, 1H), 8.57 (s, 1H), 7.91 (s, 1H), 7.66 (d, J = 5.7 Hz, |
| | | | | 1H), 7.29 (ddd, J = 14.3, 7.0, 4.4 Hz, 2H), 7.00 (dd, |
| | | | | J = 11.2, 4.8 Hz, 1H), 6.93 (d, J = 16.4 Hz, 1H), 4.58 |
| | | | | (d, J = 3.7 Hz, 1H), 3.72 (s, 2H), 3.31 (d, J = 30.1 Hz, |
| | | | | 2H), (merged with moisture) 2.04 (d, J = 33.2 Hz, |
| | | | | 2H), 1.76 (d, J = 8.7 Hz, 2H). |
| 86 | *** | ** | 496.23 | 1H NMR (400 MHz, DMSO-d6) δ 7.83 (t, J = 7.8 Hz, |
| | | | | 1H), 7.39 (br d, J = 7.3 Hz, 1H), 7.31-7.21 (m, 2H), |
| | | | | 6.98 (br t, J = 8.6 Hz, 1H), 6.83 (br d, J = 8.2 Hz, 1H), |
| | | | | 4.62 (d, J = 19.7 Hz, 2H), 4.52-4.43 (m, 1H), 3.88 |
| | | | | (s, 3H), 3.80 (dd, J = 10.5, 5.2 Hz, 2H), 3.52-3.38 |
| | | | | (m, 2H), 3.09-2.95 (m, 2H), 2.92 (t, J = 5.4 Hz, 1H), |
| | | | | 2.79 (t, J = 5.5 Hz, 1H), 2.13 (s, 1.5H), 2.11 (s, 1.5H), |
| | | | | 1.94-1.77 (m, 2H), 1.66-1.47 (m, 2H). |
| 87 | *** | ** | 497.3 | 1H NMR (400 MHz, DMSO-d6) δ 8.62 (d, J = 1.3 |
| | | | | Hz, 1H), 8.31 (d, J = 1.9 Hz, 1H), 7.31-7.21 (m, |
| | | | | 2H), 7.02-6.94 (m, 1H), 4.67 (s, 1H), 4.61 (s, 1H), |
| | | | | 4.49 (dt, J = 7.9, 4.0 Hz, 1H), 3.95 (s, 3H), 3.81 (dd, |
| | | | | J = 9.4, 5.7 Hz, 2H), 3.50-3.39 (m, 2H), 3.12-3.00 |
| | | | | (m, 2H), 2.94 (t, J = 5.8 Hz, 1H), 2.81 (t, J = 5.8 Hz, |
| | | | | 1H), 2.12 (d, J = 10.7 Hz, 3H), 1.93-1.81 (m, 2H), |
| | | | | 1.64-1.51 (m, 2H). |
| 88 | ** | ** | 499.4 | 1H NMR (400 MHz, DMSO-d6) δ as a mixture of |
| | | | | isomers 8.35 (s, 1H), 8.06 (s, 0.5H), 8.06 (s, 0.5H) |
| | | | | (1H), 7.35-7.23 (m, 2H), 7.05-6.96 (m, 1H), 4.97- |
| | | | | 4.89 (m, 1H), 4.65 (s, 1H), 4.60 (s, 1H), 4.55-4.46 |
| | | | | (m, 1H), 4.23 (t, J = 5.4 Hz, 2H), 3.82-3.72 (m, 4H), |
| | | | | 3.33-3.27 (sumerged m, 2H), 3.0-2.87 (m, 3H), |
| | | | | 2.77 (t, J = 5.7 Hz, 1H), 2.13 (s, 1.5H), 2.11 (s, 1.5H), |
| | | | | 2.09-2.01 (m, 2H), 1.90-1.78 (m, 2H). |
| 89 | ** | ** | 452.5 | 1H NMR (400 MHz, DMSO-d6) δ 13.30 (s, 1H), 9.12 |
| | | | | (s, 1H), 8.55 (d, J = 5.7 Hz, 1H), 8.40-8.11 (m, 2H), |
| | | | | 7.62 (d, J = 5.6 Hz, 1H), 7.49 (dd, J = 15.5, 8.4 Hz, |
| | | | | 1H), 7.19 (td, J = 10.2, 2.5 Hz, 1H), 7.06 (td, J = 8.4, |
| | | | | 2.2 Hz, 1H), 4.01 (d, J = 14.0 Hz, 1H), 3.75 (d, J = |
| | | | | 12.0 Hz, 1H), 3.63 (dd, J = 10.0, 4.4 Hz, 1H), 3.50 (d, |
| | | | | J = 13.0 Hz, 1H), 3.42 (d, J = 14.0 Hz, 1H), 3.37- |
| | | | | 3.28 (m, 1H, overlapping), 3.18 (s, 3H), 3.14-2.97 |
| | | | | (m, 2H), 2.83 (m, 1H), 2.76-2.69 (m, 1H), 2.43- |
| | | | | 2.34 (m, 1H). |
| 90 | ** | ** | 525 | 1H NMR (400 MHz, DMSO-d6) δ 7.54 (d, J = 6.6 |
| | | | | Hz, 1H), 7.33-7.24 (m, 2H), 7.04-6.96 (m, 1H), |
| | | | | 4.61 (d, J = 17.6 Hz, 2H), 4.55-4.45 (m, 1H), 4.21 |
| | | | | (t, J = 5.3 Hz, 2H), 3.79 (q, J = 5.7 Hz, 2H), 3.60 (s, |
| | | | | 2H), 3.43-3.35 (m, 2H), 3.05-2.94 (m, 2H), 2.90 (t, |
| | | | | J = 5.7 Hz, 1H), 2.83-2.74 (m, 3H), 2.39 (s, 3H), |
| | | | | 2.13 (s, 1.7H), 2.10 (s, 1.3H), 2.02-1.93 (m, 2H), |
| | | | | 1.76-1.64 (m, 2H). |
| 91 | ** | ** | 515 | 1H NMR (400 MHz, DMSO-d6) δ 7.35-7.25 (m, |
| | | | | 2H), 7.05-6.96 (m, 1H), 4.66-4.56 (m, 2H), 4.56- |
| | | | | 4.48 (m, 1H), 4.48-4.38 (m, 1H), 3.90-3.82 (m, |
| | | | | 1H), 3.77 (t, J = 5.7 Hz, 2H), 3.32-3.22 (overlapped |
| | | | | m, 3H), 3.21-2.93 (m, 3H), 2.93-2.83 (m, 2H), |
| | | | | 2.79-2.71 (m, 1H), 2.11 (s, 1.5H), 2.10 (s, 1.5H), |
| | | | | 2.09-2.01 (overlapped m, 2H), 2.03 (s, 1.5H), 1.99 (s, |
| | | | | 1.5H), 1.93-1.67 (m, 5H), 1.60-1.37 (m, 1H). |
| 92 | ** | * | 463 | 1H NMR (400 MHz, DMSO-d6) δ 9.10 (d, J = 0.5 |
| | | | | Hz, 1H), 8.62 (s, 1H), 8.55 (d, J = 5.8 Hz, 1H), 8.50 |
| | | | | (s, 1H), 7.55 (dd, J = 5.8, 0.5 Hz, 1H), 7.31-7.22 (m, |
| | | | | 1H), 7.04-6.93 (m, 2H), 4.62 (p, J = 6.7 Hz, 1H), |
| | | | | 3.98 (overlapping singlets, 7H), 2.74-2.57 (m, 2H), |
| | | | | 2.26-2.12 (m, 2H) |
| 93 | ** | * | 438 | 1H NMR (400 MHz, DMSO-d6) δ 13.30 (s, 1H, |
| | | | | pyrazole NH), 9.11 (s, 1H), 8.55 (d, J = 5.7 Hz, 1H), |
| | | | | 8.44 (s, 1H), 8.19 (s, 1H), 7.62 (d, J = 5.7 Hz, 1H), |
| | | | | 7.51 (dd, J = 15.5, 8.5 Hz, 1H), 7.19 (td, J = 10.4, 2.5 |
| | | | | Hz, 1H), 7.06 (td, J = 8.5, 2.2 Hz, 1H), 4.68 (t, J = 5.1 |
| | | | | Hz, 1H), 4.05 (d, J = 13.8 Hz, 1H), 3.82 (d, J = 12.6 |
| | | | | Hz, 1H), 3.76-3.68 (m, 1H), 3.53 (d, J = 12.0 Hz, |
| | | | | 1H), 3.45-3.37 (m, 2H), 3.13-2.96 (m, 2H), 2.78- |
| | | | | 2.68 (m, 1H), 2.68-2.58 (m, 1H), 2.44-2.28 (m, 1H). |
| 94 | ** | ** | 538 | 1H NMR (400 MHz, DMSO-d6) δ 9.08 (s, 1H), 8.72 |
| | | | | (s, 1H), 8.44 (s, 1H), 7.66 (s, 1H), 7.48-7.39 (m, |
| | | | | 1H), 7.21 (td, J = 10.0, 2.4 Hz, 1H), 7.06 (td, J = 8.5, |
| | | | | 2.2 Hz, 1H), 5.52 (d, J = 5.3 Hz, 1H), 4.78 (s, 1H), |
| | | | | 4.52 (d, J = 4.7 Hz, 1H), 3.92 (s, 3H), 3.52 (s, 2H), |
| | | | | 3.38 (bs, 4H, overlapped), 2.44-2.36 (m, 4H), 1.08 |
| | | | | (s, 3H), 1.05 (s, 3H). |
| 95 | ** | ** | 481 | 1H NMR (400 MHz, DMSO-d6) δ 9.07 (s, 1H), 8.73 |
| | | | | (s, 1H), 8.43 (s, 1H), 7.58 (s, 1H), 7.48-7.37 (m, J = |
| | | | | 8.5 Hz, 1H), 7.20 (td, J = 10.1, 2.5 Hz, 1H), 7.06 (td, |
| | | | | J = 8.4, 2.2 Hz, 1H), 5.59 (t, J = 5.9 Hz, 1H), 4.70 (d, |
| | | | | J = 5.8 Hz, 2H), 3.92 (s, 3H), 3.52 (s, 2H), 3.43-3.36 |
| | | | | (m, 4H, overlapped), 2.44-2.34 (m, 4H). |
| 96 | ** | ** | 410 | 1H NMR (400 MHz, DMSO-d6) δ 9.14 (s, 1H), 8.61 |
| | | | | (d, J = 3.6 Hz, 2H), 7.60 (d, J = 5.2 Hz, 1H), 7.31- |
| | | | | 7.25 (m, 2H), 7.02-6.97 (m, 1H), 4.58-4.54(m, 1H), |
| | | | | 3.65-3.62 (m, 2H), 3.32-3.21(m, 2H), 1.99-1.90 (m, |
| | | | | 2H), 1.73-1.68 (m, 2H). |
| 97 | ** | ** | 493 | 1H NMR (400 MHz, CDCl3) δ 9.20 (s, 1H), 8.59 (d, |
| | | | | J = 5.6 Hz, 1H), 8.51 (s, 1H), 8.38 (s, 1H), 7.71 (d, |
| | | | | J = 5.5 Hz, 1H), 7.00 (td, J = 9.1, 5.5 Hz, 1H), 6.90- |
| | | | | 6.84 (m, 1H), 6.82-6.77 (m, 1H), 5.49-5.46 (m, |
| | | | | 1H), 4.41 (ddd, J = 11.2, 7.5, 3.8 Hz, 1H), 4.13-4.10 |
| | | | | (m, 1H), 3.72-3.64 (m, 3H), 3.29-3.21 (m, 2H), |
| | | | | 2.15 (dd, J = 9.1, 4.0 Hz, 5H), 2.04-1.94 (m, 2H), |
| | | | | 1.78-1.71 (m, 3H), 1.66 (dd, J = 7.8, 4.2 Hz, 2H), |
| | | | | 1.26 (s, 4H) |
| 98 | * | * | 483 | 1H NMR (400 MHz, DMSO-d6) δ 12.36 (s, 1H), 7.33- |
| | | | | 7.19 (m, 2H), 7.04-6.91 (m, 1H), 4.60 (d, J = 16.6 |
| | | | | Hz, 2H), 4.50-4.42 (m, 1H), 3.80 (q, J = 6.0 Hz, 1H), |
| | | | | 3.35 (s, 2H), 3.00-2.91 (m, 2H), 2.78 (t, J = 5.7 Hz, |
| | | | | 1H), 2.16-2.10 (m, 9H), 1.86 (bs, 2H), 1.64-1.47 (m, 1H). |
| 99 | ** | ** | 436 | 1H NMR (400 MHz, MeOD) δ 11.70 (s, 1H), 9.15 |
| | | | | (s, 1H), 8.59 (d, J = 5.2 Hz, 1H), 7.86 (d, J = 5.6 Hz, |
| | | | | 1H), 7.67-7.65 (m, 1H), 7.25-7.19 (m, 2H), 7.01 (t, |
| | | | | J = 8.7 Hz, 1H), 6.57(d, J = 8.8 Hz, 1H) 4.57-4.54 (m, |
| | | | | 1H), 3.65-3.61 (m, 2H), 3.32-3.30 (m, 2H), 2.02-2.18 |
| | | | | (m, 2H), ), 1.77-1.72 (m, 2H). |
| 100 | *** | ** | 409 | 1H NMR (400 MHz, MeOD) δ 9.25 (s, 1H), 8.54 (s, |
| | | | | 3H), 8.12 (d, J = 6.1 Hz, 1H), 7.19 (td, J = 9.2, 5.4 |
| | | | | Hz, 1H), 6.99 (ddd, J = 11.4, 8.6, 3.0 Hz, 1H), 6.91- |
| | | | | 6.85 (m, 1H), 4.59-4.55 (m, 1H), 3.83-3.77 (m, |
| | | | | 2H), 3.50-3.45 (m, 2H), 2.18-2.09 (m, 2H), 2.01- |
| | | | | 1.92 (m, 2H). |
| 101 | ** | ** | 535 | 1H NMR (400 MHz, DMSO-d6) δ 7.71 (s, 1H), 7.25 |
| | | | | (qd, J = 11.6, 4.5 Hz, 2H), 7.10-6.93 (m, 2H), 6.77 |
| | | | | (s, 1H), 4.62 (d, J = 14.5 Hz, 2H), 4.47 (s, 1H), 3.77 |
| | | | | (dd, J = 23.8, 17.8 Hz, 2H), 3.46 (d, J = 12.4 Hz, 2H), |
| | | | | 3.10 (d, J = 5.9 Hz, 6H), 3.06-2.98 (m, 2H), 2.93 (s, |
| | | | | 1H), 2.79 (s, 1H), 2.12 (d, J = 12.0 Hz, 3H), 1.88 (d, |
| | | | | J = 20.8 Hz, 2H), 1.58 (d, J = 8.6 Hz, 2H). |
| 102 | *** | ** | 450 | 1H NMR (400 MHz, MeOH-d4) δ 9.07 (s, 1H), 8.50 |
| | | | | (d, J = 5.6 Hz, 1H), 7.91-7.87 (m,, 1H), 7.63 (d, J = 5.6 |
| | | | | Hz, 1H), 7.58 (d, J = 0.8 Hz, 1H), 7.16-7.10 (m, 1H), |
| | | | | 6.99-6.91 (m, 2H), 6.88-6.86 (m, 1H), 4.51-4.47 (m, |
| | | | | 1H) 3.98 (s, 3H), 3.78-3.73 (m, 2H), 3.45-3.30 (m, |
| | | | | 2H), 1.97-1.91(m, 3H), 1.80-1.72 (m, 2H). |
| 103 | ** | * | 523 | 1H NMR (400 MHz, MeOD) δ 8.10-8.01 (m, 1H), |
| | | | | 7.76 (d, J = 7.6 Hz, 1H), 7.21-7.08 (m, 2H), 6.98 |
| | | | | (ddd, J = 11.4, 8.6, 3.0 Hz, 1H), 6.90-6.83 (m, 1H), |
| | | | | 4.81 (d, J = 6.7 Hz, 2H), 4.51 (s, 1H), 4.07-4.00 (m, |
| | | | | 1H), 4.00-3.89 (m, 2H), 3.69-3.55 (m, 2H), 3.29- |
| | | | | 3.21 (m, 2H), 3.09 (t, J = 5.9 Hz, 1H), 2.98 (t, J = 5.9 |
| | | | | Hz, 1H), 2.65 (s, 1H), 2.24 (d, J = 12.0 Hz, 3H), 2.09 |
| | | | | (d, J = 20.1 Hz, 2H), 1.89 (s, 2H), 1.34 (t, J = 18.5 |
| | | | | Hz, 6H). |
| 104 | ** | ** | 462 | 1H NMR (400 MHz, DMSO-d6) δ 9.06 (s, 1H), 8.53 |
| | | | | (d, J = 5.8 Hz, 1H), 7.93 (dd, J = 8.3, 7.3 Hz, 1H), |
| | | | | 7.52 (dd, J = 5.8, 0.5 Hz, 1H), 7.43 (d, J = 6.9 Hz, |
| | | | | 1H), 7.26 (ddd, J = 11.5, 8.9, 2.9 Hz, 1H), 7.03-6.93 |
| | | | | (m, 3H), 4.67-4.55 (m, 1H), 4.00-3.87 (overlapping |
| | | | | multiplet, 4H), 3.90 (overlapping singlet, |
| | | | | 3H), 2.72-2.60 (m, 2H), 2.28-2.17 (m, 2H) |
| 105 | ** | ** | 501 | 1H NMR (400 MHz, DMSO-d6) δ 12.15 (br s, 1H), |
| | | | | 7.57-7.47 (m, 1H), 7.35-7.23 (m, 2H), 7.03-6.98 |
| | | | | (m, 1H), 6.78-6.75 (m, 1H), 4.69-4.61 (d, J = 15.7 |
| | | | | Hz, 2H), 4.56-4.47 (m, 1H), 3.86-3.75 (m, 2H), |
| | | | | 3.48-3.37 (m, 2H), 3.10-2.98 (m, 2H), 2.94 (t, J = |
| | | | | 5.6 Hz, 1H), 2.81 (t, J = 6.1 Hz, 1H), 2.13 (s, 1.7H), |
| | | | | 2.11 (s, 1.2H), 2.05-1.88 (m, 2H), 1.83-1.55 (m, 2H). |
| 106 | ** | ** | 515 | 1H NMR (400 MHz, DMSO-d6) δ 7.86-7.80 (m, |
| | | | | 1H), 7.32-7.19 (m, 2H), 7.03-6.91 (m, 2H), 4.62 |
| | | | | (d, J = 22.1 Hz, 2H), 4.50-4.39 (m, 1H), 3.83 (s, |
| | | | | 3H), 3.83-3.76 (m, 2H), 3.40 (m, 2H), 3.09-2.98 |
| | | | | (m, 2H), 2.94 (t, J = 5.9 Hz, 1H), 2.81 (t, J = 5.8 Hz, |
| | | | | 1H), 2.13 (s, 1.5H), 2.10 (s, 1.5H), 1.90-1.78 (m, |
| | | | | 2H), 1.62-1.40 (m, 2H). |
| 107 | ** | ** | 553 | 1H NMR (400 MHz, DMSO-d6) δ 8.18 (t, J = 7.6 Hz, |
| | | | | 1H), 7.34-7.23 (m, 2H), 7.04-6.96 (m, 1H), 5.06- |
| | | | | 4.90 (m, 2H), 4.69-4.56 (m, 2H), 4.56-4.44 (m, |
| | | | | 1H), 4.26 (t, J = 5.0 Hz, 1.2H), 4.15 (t, J = 5.7 Hz, |
| | | | | 0.8H), 3.98 (t, J = 4.6 Hz, 2H), 3.79 (dd, J = 10.5, 5.3 |
| | | | | Hz, 2H), 3.34-3.27 (m, 2H), 3.04-2.92 (m, 2H), |
| | | | | 2.90 (t, J = 5.7 Hz, 1.2H), 2.77 (t, J = 5.7 Hz, 0.8H), |
| | | | | 2.15 (s, 1.8H), 2.13 (s, 1.2H), 2.13 (s, 1.8H), 2.12 (s, |
| | | | | 1.2H), 2.08-1.97 (m, 2H), 1.84-1.70 (m, 2H). |
| 108 | ** | ** | 510 | 1H NMR (400 MHz, DMSO-d6) δ 8.11 (s, 1H), 7.29 |
| | | | | (ddd, J = 9.2, 7.2, 3.1 Hz, 2H), 7.05-6.96 (m, 1H), |
| | | | | 4.64-4.56 (m, 2H), 4.50 (dt, J = 7.7, 4.0 Hz, 1H), |
| | | | | 4.19-4.13 (m, 2H), 4.04 (t, J = 4.9 Hz, 2H), 3.78 (q, |
| | | | | J = 5.7 Hz, 2H), 3.41-3.27 (m, 2H), 3.13 (t, J = 5.3 |
| | | | | Hz, 2H), 3.03-2.91 (m, 2H), 2.88 (t, J = 5.7 H, 1 Hz), |
| | | | | 2.81 (submerged br. s, 1H), 2.75 (t, J = 5.7 Hz, 1H), |
| | | | | 2.12 (s, 1.7H), 2.11 (s, 1.3H), 2.08-1.97 (m, 2H), |
| | | | | 1.84-1.69 (m, 2H). |
| 109 | ** | ** | 514 | 1H NMR (400 MHz, DMSO-d6) δ 7.36-7.25 (m, |
| | | | | 2H), 7.06-6.98 (m, 1H), 4.65-4.45 (m, 4H), 3.91 |
| | | | | (d, J = 13.1 Hz, 1H), 3.76 (t, J = 5.5 Hz, 2H), 3.30- |
| | | | | 3.24 (m, 2H), 3.23-3.11 (m, 2H), 2.98 (t, J = 10.7 |
| | | | | Hz, 2H), 2.87 (t, J = 5.8 Hz, 1H), 2.74 (t, J = 5.7 Hz, |
| | | | | 1H), 2.69-2.58 (m, 1H), 2.11 (s, 1.5H), 2.10 (s, |
| | | | | 1.5H), 2.09-2.04 (overlapped m, 2H), 2.03 (s, 3H), |
| | | | | 1.89-1.77 (m, 2H), 1.77-1.65 (m, 3H), 1.62-1.46 |
| | | | | (m, 1H). |
| 110 | ** | ** | 525 | 1H NMR (400 MHz, DMSO-d6) δ 8.11 (s, 0.6H), |
| | | | | 8.09 (s, 0.4H), 7.36-7.22 (m, 2H), 7.05-6.94 (m, |
| | | | | 1H), 4.60 (overlapping d, J = 16.1 Hz, 1H), 4.58 (s, |
| | | | | 1H), 4.54-4.44 (m, 1H), 4.16 (t, J = 5.2 Hz, 2H), |
| | | | | 3.83 (s, 2H), 3.78 (q, J = 5.9 Hz, 2H), 3.44-3.28 |
| | | | | (submerged m, 2H), 3.01-2.92 (m, 2H), 2.92-2.83 |
| | | | | (m, 3H), 2.75 (t, J = 5.7 Hz, 1H), 2.44 (s, 3H), 2.13 (s, |
| | | | | 1.8H), 2.11 (m, 1.2H), 1.85-1.69 (m, 2H). |
| 111 | *** | *** | 498 | 1H NMR (400 MHz, DMSO-d6) δ 8.77-8.66 (m, |
| | | | | 1H), 7.63-7.55 (m, 1H), 7.34-7.20 (m, 2H), 7.05- |
| | | | | 6.93 (m, 1H), 4.63 (d, J = 19.8 Hz, 2H), 4.57-4.45 |
| | | | | (m, 1H), 3.95 (s, 3H), 3.81-3.80 (m, 2H), 3.58-3.41 |
| | | | | (m, 2H), 3.13-3.07 (m, 2H), 2.94 (t, J = 5.4 Hz, 1H), |
| | | | | 2.83-2.80 (m, 1H), 2.13 (s, 1.5H), 2.11 (s, 1.5H), |
| | | | | 1.93-1.90 (m, 2H), 1.72-1.51 (m, 2H). |
| 112 | ** | ** | 496 | 1H NMR (400 MHz, DMSO-d6) δ 8.25 (dt, J = 4.9, |
| | | | | 1.9 Hz, 2H), 7.83 (dd, J = 7.3, 1.9 Hz, 2H), 7.30- |
| | | | | 7.19 (m, 2H), 7.16-7.11 (m, 1H), 7.01-6.94 (m, |
| | | | | 1H), 4.63 (s, 1H), 4.58 (s, 1H), 4.47-4.37 (m, J = 3.9 |
| | | | | Hz, 1H), 3.85 (s, 1.5H), 3.85 (s, 1.5H), 3.83-3.77 |
| | | | | (m, J = 5.5 Hz, 2H), 3.39-3.33 (submerge m, 2H), |
| | | | | 3.01-2.87 (m, 3H), 2.79 (t, J = 5.8 Hz, 1H), 2.13 (s, |
| | | | | 1.5H), 2.10 (s, 1.5H), 1.87-1.68 (m, J = 9.8 Hz, 2H), |
| | | | | 1.50-1.37 (m, 2H). |
| 113 | ** | * | 496 | 1H NMR (400 MHz, DMSO-d6) δ 7.81 (dt, J = 6.7, |
| | | | | 2.1 Hz, 1H), 7.57 (dt, J = 6.9, 2.4 Hz, 1H), 7.30-7.20 |
| | | | | (m, 2H), 7.02-6.94 (m, 1H), 6.33 (t, J = 6.8 Hz, 1H), |
| | | | | 4.59 (s, 1H), 4.54 (s, 1H), 4.47-4.39 (m, 1H), 3.83- |
| | | | | 3.74 (m, 2H), 3.53-3.46 (m, 5H), 3.05-2.96 (m, |
| | | | | 2H), 2.89 (t, J = 5.8 Hz, 1H), 2.76 (t, J = 5.8 Hz, 1H), |
| | | | | 2.12 (s, 1.5 H), 2.09 (s, 1.5H), 1.89-1.79 (m, 2H), |
| | | | | 1.55-1.45 (m, 2H). |
| 114 | *** | ** | 498 | 1H NMR (400 MHz, DMSO-d6) δ 8.68 (s, 1H), 8.41 |
| | | | | (s, 1H), 7.30-7.23 (m, 2H), 7.01-6.96 (m, 1H), |
| | | | | 4.63 (J = 20.3 Hz, d, 2H), 4.49-4.45 (m, 1H), 3.97 |
| | | | | (s, 3H), 3.82-3.78 (m, 2H), 3.43-3.39 (m, 2H), |
| | | | | 3.03-3.00 (m, 2H), 2.94 (t, J = 5.7 Hz, 1H), 2.80 (t, |
| | | | | J = 5.7 Hz, 1H), 2.13 (s, 1.5H), 2.10 (s, 1.5H), 1.91- |
| | | | | 1.88 (m, 2H), 1.63-1.57 (m, 2H). |
| 115 | *** | ** | 454 | 1H NMR (400 MHz, DMSO-d6) δ 13.10 (s, 1H), 8.30 |
| | | | | (br s, 1H), 8.14 (br s, 1H), 7.33-7.25(m, 2H), 7.01 (t, |
| | | | | J = 8.8 Hz, 1H), 4.60 (d, J = 15.5 Hz, 2H), 4.51 (s, |
| | | | | 1H), 3.80 (d, J = 3.1 Hz, 2H), 3.00-2.90 (m, 3H), |
| | | | | 2.79 (s, 1H), 2.12 (d, J = 4.8 Hz, 3H), 2.04 (s, 2H), |
| | | | | 1.79 (dd, J = 14.3, 10.2 Hz, 2H). |
| 116 | *** | ** | 469 | 1H NMR (400 MHz, MeOD) δ 7.68 (d, J = 4.0 Hz, |
| | | | | 1H), 7.17 (td, J = 9.2, 5.4 Hz, 1H), 6.97 (ddd, J = |
| | | | | 11.4, 8.6, 3.0 Hz, 1H), 6.90-6.82 (m, 1H), 4.75 (d, |
| | | | | J = 4.8 Hz, 2H), 4.47 (s, 1H), 3.93 (t, J = 6.0 Hz, 1H), |
| | | | | 3.88 (t, J = 6.0 Hz, 1H), 3.47 (d, J = 3.4 Hz, 3H), 3.09 |
| | | | | (ddd, J = 16.0, 11.1, 4.9 Hz, 3H), 3.00 (t, J = 6.0 Hz, |
| | | | | 1H), 2.90 (t, J = 5.9 Hz, 1H), 2.46 (s, 3H), 2.22 (d, J = |
| | | | | 8.7 Hz, 3H), 2.09 (s, 3H), 1.92 (d, J = 8.4 Hz, 3H). |
| 117 | *** | ** | 495 | 1H NMR (400 MHz, MeOD) δ 7.61-7.55 (m, 1H), |
| | | | | 7.11 (td, J = 9.2, 5.4 Hz, 1H), 6.95 (ddd, J = 11.5, 6.5, |
| | | | | 2.2 Hz, 2H), 6.88-6.80 (m, 1H), 6.52 (dd, J = 8.3, |
| | | | | 1.6 Hz, 1H), 4.71 (d, J = 7.7 Hz, 2H), 4.39 (s, 1H), |
| | | | | 3.93 (t, J = 6.0 Hz, 1H), 3.88 (t, J = 6.0 Hz, 1H), 3.51 |
| | | | | (s, 2H), 3.14-3.04 (m, 2H), 2.99 (t, J = 5.8 Hz, 1H), |
| | | | | 2.92 (t, J = 3.1 Hz, 3H), 2.89 (d, J = 5.9 Hz, 1H), 2.22 |
| | | | | (d, J = 11.4 Hz, 3H), 1.90 (s, 2H), 1.75-1.65 (m, 2H). |
| 118 | ** | ** | 535 | 1H NMR (400 MHz, MeOD) δ 8.43 (s, 1H), 8.19 (s, |
| | | | | 1H), 7.16 (td, J = 9.0, 5.2 Hz, 1H), 6.97 (ddd, J = |
| | | | | 11.5, 8.7, 3.1 Hz, 1H), 6.90-6.82 (m, 1H), 5.48 (dd, |
| | | | | J = 9.8, 2.5 Hz, 1H), 4.49-4.41 (m, 1H), 4.06 (d, J = |
| | | | | 11.7 Hz, 1H), 3.91 (s, 2H), 3.78 (d, J = 10.5 Hz, 2H), |
| | | | | 3.75 (dd, J = 11.5, 2.9 Hz, 1H), 3.41 (s, 2H), 3.09- |
| | | | | 3.01 (m, 2H), 2.98 (s, 4H), 2.19-2.02 (m, 5H), 1.90 |
| | | | | (d, J = 9.0 Hz, 2H), 1.83-1.68 (m, 2H), 1.65 (s, 1H). |
| 119 | *** | ** | 451 | 1H NMR (400 MHz, MeOD) δ 8.28 (s, 2H), 7.16 (td, |
| | | | | J = 9.2, 5.4 Hz, 1H), 6.97 (ddd, J = 11.4, 8.7, 3.0 Hz, |
| | | | | 1H), 6.91-6.80 (m, 1H), 4.45 (dd, J = 7.8, 3.9 Hz, |
| | | | | 1H), 3.92 (s, 2H), 3.80 (s, 2H), 3.42 (dd, J = 10.1, 5.5 |
| | | | | Hz, 2H), 3.08-3.01 (m, 2H), 3.01-2.95 (m, 4H), |
| | | | | 2.14-2.04 (m, 2H), 1.90 (ddd, J = 17.7, 8.5, 4.2 Hz, 2H). |
| 120 | *** | ** | 526 | 1H NMR (400 MHz, DMSO-d6) δ 13.09 (s, 1H), 8.33 |
| | | | | (s, 1H), 8.11 (s, 1H), 7.34-7.24 (m, 2H), 7.01 (dd, |
| | | | | J = 14.3, 4.9 Hz, 1H), 4.50 (d, J = 3.5 Hz, 1H), 4.38 (s, |
| | | | | 2H), 3.63-3.56 (m, 4H), 3.53 (t, J = 5.8 Hz, |
| | | | | 2H), 3.31(s, 2H), 3.22 (d, J = 4.4 Hz, 4H), 2.95 (t, J = |
| | | | | 9.5 Hz, 2H), 2.87 (dd, J = 11.0, 5.6 Hz, 2H), 2.04 (s, |
| | | | | 2H), 1.80 (d, J = 9.5 Hz, 2H). |
| 121 | *** | ** | 499 | 1H NMR (400 MHz, MeOD) δ 8.30 (s, 1H), 7.83 (dd, |
| | | | | J = 18.5, 8.0 Hz, 1H), 7.49-7.31 (m, 1H), 7.23- |
| | | | | 7.13 (m, 1H), 6.97 (ddd, J = 11.4, 8.6, 3.0 Hz, 1H), |
| | | | | 6.87 (dd, J = 14.1, 4.9 Hz, 1H), 4.94 (dd, J = 11.5, 5.3 |
| | | | | Hz, 0.5H), 4.63 (s, 2H), 4.50-4.39 (m, 1H), 3.91 (t, |
| | | | | J = 6.3 Hz, 1H), 3.81 (t, J = 5.8 Hz, 1H), 3.51 (t, J = 6.4 |
| | | | | Hz, 1H), 3.50-3.39 (m, 1H), 3.10-2.99 (m, 1H), |
| | | | | 2.90 (t, J = 5.8 Hz, 2H), 2.08 (s, 2H), 1.90 (dd, J = |
| | | | | 8.8, 4.0 Hz, 2H), 1.34 (dd, J = 39.2, 10.8 Hz, 9H). |
| 122 | *** | ** | 485 | 1H NMR (400 MHz, DMSO-d6) δ 8.29 (s, 1H), |
| | | | | 7.18-7.12 (m, 1H), 6.99-6.94 (m, 1H), 6.88- |
| | | | | 6.83(m, 1H), 4.63(s, 1H), 4.44-4.16 (m, 1H), 4.2 (q, |
| | | | | j = 6.8 Hz, 2H), 3.82-3.45 (m, 2H), 3.48-3.40(m, 1H), |
| | | | | 3.10-3.01 (m, 2H), 2.92-2.89 (t, J = 6 Hz, 2H), 2.10- |
| | | | | 2.06 (m, 2H), 1.91-1.88 (m, 2H), 1.32-1.30 (m, 6H). |
| 123 | *** | ** | 505 | 1H NMR (400 MHz, DMSO-d6) δ 8.22 (s, 1H), 7.35- |
| | | | | 7.21 (m, 2H), 7.00 (ddt, J = 13.7, 11.9, 5.3 Hz, 1H), |
| | | | | 4.55-4.48 (m, 1H), 4.42 (s, 1H), 3.63 (t, J = 5.9 Hz, |
| | | | | 2H), 3.37-3.30 (m, 2H), 3.21 (q, J = 7.3 Hz, 2H), |
| | | | | 3.01-2.88 (m, 4H), 2.06 (d, J = 12.3 Hz, 2H), 1.86- |
| | | | | 1.76 (m, 2H), 1.23 (t, J = 7.3 Hz, 3H). |
| 124 | ** | ** | 413 | 1H NMR (400 MHz, DMSO-d6) δ 9.40 (s, 1H), 8.23 |
| | | | | (s, 2H), 7.36-7.25 (m, 2H), 7.02 (ddd, J = 9.4, 3.1, |
| | | | | 1.5 Hz, 1H), 4.57-4.48 (m, 1H), 4.28 (s, 1H), 3.51 |
| | | | | (d, J = 4.3 Hz, 2H), 3.40-3.31 (m, 2H), 3.01 (dt, J = |
| | | | | 12.9, 7.7 Hz, 4H), 2.06 (d, J = 10.0 Hz, 2H), 1.88- |
| | | | | 1.73 (m, 2H). |
| 125 | *** | ** | 555 | 1H NMR (400 MHz, DMSO-d6) δ 8.42 (s, 1H), 8.11 |
| | | | | (s, 1H), 7.29 (ddd, J = 14.3, 6.8, 4.4 Hz, 2H), 7.01 (t, |
| | | | | J = 8.7 Hz, 1H), 5.56-5.48 (m, 1H), 4.61-4.48 (m, |
| | | | | 3H), 3.94 (d, J = 11.5 Hz, 1H), 3.74 (t, J = 5.8 Hz, |
| | | | | 2H), 3.64 (d, J = 10.3 Hz, 4H), 2.97 (dd, J = 12.5, 9.1 |
| | | | | Hz, 2H), 2.84 (t, J = 5.6 Hz, 2H), 2.18-2.01 (m, 4H), |
| | | | | 1.96 (d, J = 10.0 Hz, 2H), 1.82 (d, J = 9.5 Hz, 2H), |
| | | | | 1.68 (d, J = 6.5 Hz, 1H), 1.56 (s, 2H). |
| 126 | *** | ** | 496 | 1H NMR (400 MHz, DMSO-d6) δ 8.72 (d, J = 2.2 |
| | | | | Hz, 1H), 8.28-8.19 (m, 1H), 7.32-7.21 (m, 2H), |
| | | | | 7.05-6.89 (m, 2H), 4.66 (s, 0.9H), 4.62 (s, 1.1H), |
| | | | | 4.50-4.41 (m, 1H), 3.91 (s, 3H), 3.80 (q, J = 5.5 Hz, |
| | | | | 2H), 3.39-3.35 (overlapping m, 2H), 3.03-2.86 (m, |
| | | | | 3H), 2.79 (t, J = 5.7 Hz, 1H), 2.13 (s, 1.7H), 2.11 (s, |
| | | | | 1.3H), 2.00-1.87 (m, 2H), 1.73-1.59 (m, 2H) |
| 127 | * | * | 436 | 1H NMR (400 MHz, DMSO-d6) δ 9.15 (s, 1H), 8.60 |
| | | | | (d, J = 5.7 Hz, 1H), 8.47 (s, 1H), 8.11 (s, 1H), 7.65 (d, |
| | | | | J = 5.7 Hz, 1H), 7.37-7.28 (m, 1H), 7.21 (td, J = |
| | | | | 10.5, 2.4 Hz, 1H), 6.95 (td, J = 8.5, 1.9 Hz, 1H), 4.56 |
| | | | | (s, 2H), 4.10 (s, 2H), 3.94 (s, 3H), 3.72 (t, J = 5.4 Hz, |
| | | | | 2H), 3.28 (t, J = 5.4 Hz, 2H). |
| 128 | ** | * | 436 | 1H NMR (400 MHz, DMSO-d6) δ 9.14 (s, 1H), 8.58 |
| | | | | (d, J = 5.7 Hz, 1H), 8.54 (s, 1H), 8.19 (s, 1H), 7.64 (d, |
| | | | | J = 5.7 Hz, 1H), 7.52 (dd, J = 14.9, 8.1 Hz, 1H), 7.39 |
| | | | | (td, J = 9.8, 2.3 Hz, 1H), 7.21 (td, J = 8.5, 2.3 Hz, |
| | | | | 1H), 3.95 (s, 3H), 3.87-3.83 (m, 2H), 3.49-3.42 |
| | | | | (m, 4H), 3.37-3.34 (submerged m, 2H). |
| 129 | ** | ** | 505 | 1H NMR (400 MHz, DMSO-d6) δ 8.22 (bs, formate |
| | | | | H), 7.78 (d, J = 29.8 Hz, 1H), 7.75-7.62 (m, 2H), |
| | | | | 7.39 (dd, J = 11.9, 4.2 Hz, 1H), 7.28-7.14 (m, 3H), |
| | | | | 6.94 (dd, J = 12.3, 5.1 Hz, 1H), 4.67 (d, J = 19.6 Hz, |
| | | | | 2H), 4.42-4.33 (m, 1H), 3.84 (dd, J = 10.2, 5.6 Hz, |
| | | | | 2H), 3.26-3.19 (bs, 2H), 3.00 (t, J = 5.8 Hz, 1H), |
| | | | | 2.97-2.85 (m, 3H), 2.15 (s, 1.5H), 2.11 (s, 1.5H), |
| | | | | 1.78-1.62 (m, 2H), 1.46-1.29 (m, 2H). |
| 130 | * | * | 431 | 1H NMR (400 MHz, DMSO-d6) δ 13.37 (br. s, 1H), |
| | | | | 9.17 (s, 1H), 8.59 (d, J = 5.7 Hz, 1H), 8.57-8.08 (m, |
| | | | | 2H), 7.67 (d, J = 5.6 Hz, 1H), 7.66-7.60 (m, 1H), |
| | | | | 7.44 (ddd, J = 10.7, 9.5, 2.5 Hz, 1H), 7.23 (td, J = 8.4, |
| | | | | 2.3 Hz, 1H), 6.95 (s, 1H), 4.68 (s, 2H), 4.07 (t, J = 5.2 |
| | | | | Hz, 2H), 3.81 (t, J = 5.3 Hz, 2H). |
| 131 | ** | ** | 537 | 1H NMR (400 MHz, DMSO-d6) δ 8.04 (td, J = 7.8, |
| | | | | 1.6 Hz, 1H), 7.90 (dd, J = 7.9, 0.7 Hz, 1H), 7.57 (d, |
| | | | | J = 7.2 Hz, 1H), 7.31-7.18 (m, 2H), 7.04-6.88 (m, |
| | | | | 1H), 4.78-4.54 (m, 2H), 4.46 (dt, J = 8.0, 4.1 Hz, |
| | | | | 1H), 3.81 (dd, J = 10.3, 5.6 Hz, 2H), 3.48-3.37 (m, |
| | | | | 2H), 3.02 (s, 3H), 3.00-2.90 (overlapping m, 6H), |
| | | | | 2.81 (t, J = 5.7 Hz, 1H), 2.14 (s, 1.6H), 2.11 (s, 1.4H), |
| | | | | 1.94-1.76 (m, 2H), 1.69-1.44 (m, 2H); ~1:1 |
| | | | | mixture of rotamers |
| 132 | ** | ** | 523 | 1H NMR (400 MHz, DMSO-d6) δ 8.50 (q, J = 4.5 |
| | | | | Hz, 1H), 8.12 (td, J = 7.7, 2.3 Hz, 1H), 8.03 (dd, J = |
| | | | | 7.7, 1.2 Hz, 1H), 7.98 (dd, J = 7.7, 1.0 Hz, 1H), 7.32- |
| | | | | 7.18 (m, 2H), 7.02-6.89 (m, 1H), 4.69-4.60 (m, |
| | | | | 2H), 4.49-4.38 (m, 1H), 3.81 (q, J = 5.6 Hz, 2H), |
| | | | | 3.46-3.35 (m, 2H), 3.04-2.90 (m, 3H), 2.86 (s, |
| | | | | 1.4H), 2.85 (s, 1.6H), 2.81 (t, J = 5.8 Hz, 1H), 2.14 (s, |
| | | | | 1.6H), 2.11 (s, 1.4H), 1.90-1.76 (m, 2H), 1.65-1.47 |
| | | | | (m, 2H) |
| 133 | ** | ** | 496 | 1H NMR (400 MHz, DMSO-d6) δ 7.92 (t, J = 7.7 Hz, |
| | | | | 1H), 7.67 (d, J = 7.8 Hz, 1H), 7.48 (d, J = 7.7 Hz, |
| | | | | 1H), 7.32-7.17 (m, 2H), 7.04-6.92 (m, 1H), 5.49 (t, |
| | | | | J = 5.6 Hz, 1H), 4.67-4.57 (m, 4H), 4.53-4.39 (m, |
| | | | | 1H), 3.80 (q, J = 5.6 Hz, 2H), 3.46-3.36 |
| | | | | (overlapping m, 2H), 3.06-2.88 (m, 3H), 2.79 (t, J = |
| | | | | 5.7 Hz, 1H), 2.13 (s, 1.6H), 2.10 (s, 1.4H), 1.93- |
| | | | | 1.77 (m, 2H), 1.67-1.48 (m, 2H) |
| 134 | ** | ** | 510 | 1H NMR (400 MHz, DMSO-d6) δ 8.47 (s, 1H), 8.17- |
| | | | | 8.12 (m, 1H), 7.34-7.24 (m, 2H), 7.05-6.95 (m, |
| | | | | 1H), 5.34 (p, J = 7.4 Hz, 1H), 4.63 (d, J = 15.5 Hz, |
| | | | | 2H), 4.55-4.47 (m, 1H), 4.03-3.91 (m, 2H), 3.87- |
| | | | | 3.73 (m, 4H), 3.41-3.22 (m, 2H, overlapped), 2.97 |
| | | | | (t, J = 9.9 Hz. 2H), 2.91 (t, J = 5.6 Hz, 1H), 2.77 (t, |
| | | | | J = 5.5 Hz, 1H), 2.13 (s, 1.5H, overlapped), 2.11 (s, |
| | | | | 1.5H, overlapped), 2.10-2.01 (m, 3H, overlapped), |
| | | | | 1.90-1.73 (m, 2H). |
| 135 | *** | ** | 473 | 1H NMR (400 MHz, DMSO-d6) δ 12.68 (bs, 1H), |
| | | | | 8.25 (d, J = 1.5 Hz, 1H), 7.31-7.25 (m, 2H), 7.06- |
| | | | | 6.93 (m, 1H), 4.60 (d, J = 17.5 Hz, 2H), 4.49-4.46 |
| | | | | (m, 1H), 3.82-3.74 (m, 2H), 3.42-3.34 |
| | | | | (overlapping m, 2H), 2.93 (dt, J = 11.8, 8.4 Hz, 3H), |
| | | | | 2.76 (t, J = 5.6 Hz, 1H), 2.12 (s, 1.5H), 2.11 (s, 1.5H), |
| | | | | 2.04-1.93 (m, 2H), 1.81-1.62 (m, 2H). |
| 136 | *** | ** | 500 | 1H NMR (400 MHz, DMSO-d6) δ 9.07-9.03 |
| | | | | (m, 1H), 8.67 (t, J = 2.6 Hz, 1H), 8.37 (s, 1H), 8.32* |
| | | | | (s, 0.1H), 7.32-7.22 (m, 2H), 7.03-6.95 (m, 1H), |
| | | | | 4.69 (s, 0.9H), 4.65 (s, 1.1H), 4.55-4.41 (m, 1H), |
| | | | | 3.85-3.77 (m, 2H), 3.43-3.35 (overlapped m, 2H), |
| | | | | 3.04-2.92 (m, 3H), 2.82 (t, J = 5.7 Hz, 1H), 2.14 (s, |
| | | | | 1.7H), 2.11 (s, 1.3H), 1.98-1.86 (m, 2H), 1.70-1.58 |
| | | | | (m, 2H). |
| 137 | *** | ** | 500 | 1H NMR (400 MHz, DMSO-d6) δ 8.00 (td, J = 7.8, |
| | | | | 1.9 Hz, 1H), 7.89 (d, J = 7.5 Hz, 1H), 7.54 (dd, J = |
| | | | | 7.8, 2.6 Hz, 1H), 7.31-7.22 (m, 2H), 7.02-6.94 (m, |
| | | | | 1H), 4.66 (s, 0.9H), 4.61 (s, 1.1H), 4.53-4.44 (m, |
| | | | | 1H), 3.80 (dd, J = 10.1, 5.5 Hz, 2H), 3.47-3.37 (m, |
| | | | | 2H), 3.01 (t, J = 10.7 Hz, 2H), 2.93 (t, J = 5.7 Hz, |
| | | | | 1H), 2.80 (t, J = 5.8 Hz, 1H), 2.14 (s, 1.7H), 2.11 (s, |
| | | | | 1.3H), 1.96-1.84 (m, 2H), 1.69-1.57 (m, 2H). |
| 138 | ** | ** | 526 | 1H NMR (400 MHz, DMSO-d6) δ 8.41 (s, 1H), 8.03 |
| | | | | (d, J = 2.1 Hz, 1H), 7.33-7.24 (m, 2H), 7.04-6.96 |
| | | | | (m, 1H), 4.64 (s, 1H), 4.60 (s, 1H), 4.55-4.47 (m, |
| | | | | 1H), 4.27 (t, J = 6.1 Hz, 2H), 3.82-3.73 (m, 2H), |
| | | | | 3.36-3.26 (m, 2H), 2.98-2.85 (m, 3H), 2.76 (t, J = |
| | | | | 5.6 Hz, 1H), 2.67 (t, J = 6.2 Hz, 2H), 2.18 (s, 6H), |
| | | | | 2.12 (s, 1.6H), 2.11 (s, 1.4H), 2.10-2.01 (m, 2H), |
| | | | | 1.91-1.78 (m, 2H). |
| 139 | ** | ** | 427 | 1H NMR (400 MHz, DMSO-d6) δ 8.24-8.20 (bs, |
| | | | | 2H), 8.15 (s, 1H)7.32-7.25 (m, 2H)), 7.06-6.96 (m, |
| | | | | 1H), 4.55-4.45 (m, 1H), 3.98 (s, 1H), 3.53 (s, 2H), |
| | | | | 3.38-3.26 (m, 2H), , 2.99-2.89 (m, 2H), 2.83 (t, J = |
| | | | | 5.5 Hz, 2H), 2.75-2.66 (m, 2H), 2.40 (s, 3H), 2.06 |
| | | | | (d, J = 14.9 Hz, 2H), 1.88-1.74 (m, 2H). |
| 140 | ** | ** | 541 | 1H NMR (400 MHz, DMSO-d6) δ 8.40 (s, 1H), 8.09 |
| | | | | (s, 1H), 7.33 (ddd, J = 11.7, 9.2, 4.2 Hz, 2H), 7.02 |
| | | | | (dd, J = 13.3, 4.0 Hz, 1H), 5.51 (d, J = 7.9 Hz, 1H), |
| | | | | 4.49 (t, J = 5.4 Hz, 2H), 3.94 (d, J = 11.7 Hz, 1H), |
| | | | | 3.70-3.56 (m, 6H), 2.94-2.92 (m, 2H), 2.82 (s, |
| | | | | 4H), 2.67-2.61 (m, J = 3.6, 2H), 2.33-2.32 (m, 1H), |
| | | | | 2.14-2.04 (m, 2H), 1.96 (d, J = 10.2 Hz, 2H), 1.83 |
| | | | | (d, J = 5.7 Hz, 3H), 1.55 (s, 2H). |
| 141 | ** | ** | 457 | 1H NMR (400 MHz, DMSO-d6) δ 8.18 (s, 2H), 7.32- |
| | | | | 7.25 (m, 2H), 7.01 (t, J = 8.7 Hz, 1H), 4.49 (t, J = |
| | | | | 5.1 Hz, 1H), 3.83 (s, 1H), 3.60 (dd, J = 13.5, 7.9 Hz, |
| | | | | 3H), 3.01 (t, J = 6.0 Hz, 1H), 2.97-2.89 (m, 2H), |
| | | | | 2.81 (s, 4H), 2.72 (s, 1H), 2.67 (s, 1H), 2.63 (t, J = 6.1 |
| | | | | Hz, 1H), 2.33 (s, 1H), 2.04 (s, 1H), 1.83 (dd, J = 24.9, |
| | | | | 20.5 Hz, 2H). |
| 142 | ** | ** | 555 | 1H NMR (400 MHz, DMSO-d6) δ 8.40 (s, 1H), 8.09 |
| | | | | (s, 1H), 7.29 (ddd, J = 11.7, 9.1, 4.2 Hz, 2H), 7.08- |
| | | | | 6.94 (m, 1H), 5.51 (dd, J = 9.8, 1.9 Hz, 1H), 4.50 (dd, |
| | | | | J = 7.7, 3.9 Hz, 1H), 3.94 (d, J = 11.3 Hz, 1H), 3.73- |
| | | | | 3.61 (m, 3H), 3.54 (t, J = 5.7 Hz, 2H), 3.27 (s, 3H), |
| | | | | 2.95 (dd, J = 12.4, 9.0 Hz, 2H), 2.82 (s, 4H), 2.72 (t, |
| | | | | J = 5.7 Hz, 2H), 2.20-2.01 (m, 3H), 1.95 (d, J = 10.2 |
| | | | | Hz, 2H), 1.88-1.75 (m, 2H), 1.74-1.52 (m, 3H). |
| 143 | *** | ** | 471 | 1H NMR (400 MHz, DMSO-d6) δ 13.05 (s, 1H), 8.14 |
| | | | | (s, 2H), 7.29 (ddd, J = 11.7, 9.2, 4.2 Hz, 2H), 7.05- |
| | | | | 6.97 (m, 1H), 4.54-4.46 (m, 1H), 3.63 (s, 2H), 3.54 |
| | | | | (t, J = 5.7 Hz, 2H), 3.32 (s, 3H), (2.98-2.89 (m, |
| | | | | 2H), 2.81 (s, 3H), 2.72 (t, J = 5.7 Hz, 2H), 2.04 (s, |
| | | | | 2H), 1.81 (dt, J = 13.4, 10.0 Hz, 2H). |
| 144 | ** | ** | 568 | 1H NMR (400 MHz, MeOD) δ 8.42 (s, 1H), 8.18 (s, |
| | | | | 1H), 7.16 (td, J = 9.2, 5.4 Hz, 1H), 6.97 (ddd, J = |
| | | | | 11.4, 8.6, 3.0 Hz, 1H), 6.86 (ddd, J = 11.0, 3.9, 2.1 |
| | | | | Hz, 1H), 5.47 (dd, J = 9.7, 2.4 Hz, 1H), 4.57 (s, 1H), |
| | | | | 4.48-4.41 (m, 1H), 4.06 (d, J = 11.7 Hz, 1H), 3.80- |
| | | | | 3.72 (m, 3H), 3.43 (dd, J = 19.7, 12.1 Hz, 2H), 3.36- |
| | | | | 3.23 (m, 2H), 3.07-2.95 (m, 4H), 2.92 (t, J = 5.4 Hz, |
| | | | | 2H), 2.78 (s, 2H), 2.21-1.99 (m, 5H), 1.98-1.83 |
| | | | | (m, 2H), 1.83-1.57 (m, 3H), 1.29 (s, 2H). |
| 145 | ** | ** | 484 | 1H NMR (400 MHz, MeOD) δ 8.27 (s, 2H), 7.16 (td, |
| | | | | J = 9.2, 5.4 Hz, 1H), 6.97 (ddd, J = 11.4, 8.6, 3.0 Hz, |
| | | | | 1H), 6.87 (ddd, J = 11.0, 3.9, 2.4 Hz, 1H), 4.47 (dd, |
| | | | | J = 10.5, 6.7 Hz, 3H), 4.07 (s, 2H), 3.65 (d, J = 16.1 |
| | | | | Hz, 2H), 3.53-3.44 (m, 2H), 3.21 (m, 2H), 3.16- |
| | | | | 3.04 (m, 2H), 2.85 (d, J = 9.0 Hz, 3H), 2.15-2.05 |
| | | | | (m, 2H), 1.91 (ddd, J = 17.5, 8.1, 4.3 Hz, 2H). |
| 146 | ** | ** | 582 | 1H NMR (400 MHz, DMSO-d6) δ 8.41 (s, 1H), 8.10 |
| | | | | (s, 1H), 7.84 (d, J = 4.7 Hz, 1H), 7.34-7.25 (m, 2H), |
| | | | | 7.04-6.97 (m, 1H), 5.55-5.47 (m, 1H), 4.51 (s, |
| | | | | 1H), 3.94 (d, J = 11.9 Hz, 1H), 3.78-3.61 (m, 1H), |
| | | | | 3.42-3.24 (m, 2H), 2.95 (dd, J = 12.8, 9.4 Hz, 3H), |
| | | | | 2.86 (d, J = 4.5 Hz, 2H), 2.79 (d, J = 5.0 Hz, 2H), |
| | | | | 2.69-2.66 (m, 2H), 2.61 (d, J = 4.7 Hz, 3H), 2.14 (d, |
| | | | | J = 11.6 Hz, 1H), 2.05 (s, 3H), 1.95 (d, J = 10.0 Hz, |
| | | | | 2H), 1.82 (d, J = 9.3 Hz, 2H), 1.60 (s, 1H), 1.56 (d, |
| | | | | J = 3.4 Hz, 2H), 1.20 (d, J = 6.9 Hz, 3H). |
| 147 | ** | ** | 498 | 1H NMR (400 MHz, MeOD) δ 8.28 (s, 2H), 7.16 (td, |
| | | | | J = 9.2, 5.4 Hz, 1H), 6.97 (ddd, J = 11.4, 8.6, 3.0 Hz, |
| | | | | 1H), 6.90-6.83 (m, 1H), 4.56-4.41 (m, 2H), 4.09 |
| | | | | (dd, J = 13.1, 6.2 Hz, 1H), 3.75-3.60 (m, 1H), 3.49 |
| | | | | (dd, J = 9.7, 5.1 Hz, 1H), 3.21 (dd, J = 14.6, 7.3 Hz, |
| | | | | 4H), 3.11 (dd, J = 10.8, 7.0 Hz, 2H), 2.85 (s, 3H), |
| | | | | 2.14-2.04 (m, 2H), 1.90 (dt, J = 12.8, 6.6 Hz, 2H), |
| | | | | 1.68 (d, J = 6.9 Hz, 3H), 1.30 (dd, J = 12.2, 4.9 Hz, 4H). |
| 148 | **** | ** | 485 | 1H NMR (400 MHz, MeOD) δ 8.41-8.16 (m, 1H), |
| | | | | 7.16 (td, J = 9.2, 5.4 Hz, 1H), 6.97 (ddd, J = 11.4, 8.6, |
| | | | | 3.0 Hz, 1H), 6.90-6.82 (m, 1H), 4.73 (s, 1H), 4.70- |
| | | | | 4.64 (m, 1H), 4.50-4.42 (m, 1H), 4.28 (d, J = 10.9 |
| | | | | Hz, 1H), 3.98-3.81 (m, 1H), 3.50-3.38 (m, 5H), |
| | | | | 3.10-2.94 (m, 3H), 2.94-2.86 (m, 1H), 2.08 (dd, |
| | | | | J = 9.8, 6.3 Hz, 2H), 1.97-1.83 (m, 1H). |
| 149 | *** | ** | 540 | 1H NMR (400 MHz, MeOD) δ 8.28 (d, J = 4.7 Hz, |
| | | | | 2H), 7.16 (td, J = 9.2, 5.4 Hz, 1H), 6.97 (ddd, J = |
| | | | | 11.4, 8.6, 3.0 Hz, 1H), 6.90-6.83 (m, 1H), 4.78 (s, |
| | | | | 1H), 4.63 (s, 1H), 4.45 (dd, J = 14.3, 7.4 Hz, 3H), |
| | | | | 4.11-3.76 (m, 7H), 3.58 (s, 1H), 3.46 (dd, J = 9.1, |
| | | | | 5.0 Hz, 2H), 3.29-3.24 (m, 1H), 3.10-3.02 (m, |
| | | | | 3H), 2.94 (t, J = 5.9 Hz, 1H), 2.14-2.03 (m, 2H), |
| | | | | 1.95-1.83 (m, 2H). |
| 150 | **** | ** | 519 | 1H NMR (400 MHz, MeOD) δ 8.29 (s, 2H), 7.19- |
| | | | | 7.12 (m, 1H), 6.97 (ddd, J = 11.5, 8.7, 3.1 Hz, 1H), |
| | | | | 6.89-6.82 (m, 1H), 4.53 (s, 1H), 4.48-4.42 (m, |
| | | | | 1H), 3.74 (t, J = 5.9 Hz, 1H), 3.49-3.38 (m, 2H), |
| | | | | 3.08-3.01 (m, 1H), 2.98 (t, J = 5.9 Hz, 2H), 2.14- |
| | | | | 2.05 (m, 2H), 1.95-1.83 (m, 2H), 1.36 (d, J = 6.8 |
| | | | | Hz, 6H). |
| 151 | ** | ** | 617 | 1H NMR (400 MHz, DMSO-d6) δ 8.42 (s, 1H), 8.10 |
| | | | | (s, 1H), 7.29 (ddd, J = 11.7, 9.1, 4.3 Hz, 2H), 7.05- |
| | | | | 6.97 (m, 1H), 5.54-5.50 (m, 1H), 4.92 (dd, J = 14.6, |
| | | | | 7.5 Hz, 1H), 4.77 (dt, J = 22.9, 6.7 Hz, 4H), 3.94 (d, |
| | | | | J = 11.4 Hz, 1H), 3.65 (dt, J = 11.9, 6.4 Hz, 3H), 3.00 |
| | | | | (d, J = 9.3 Hz, 2H), 2.89 (t, J = 5.9 Hz, 2H), 2.10 (dd, |
| | | | | J = 30.5, 11.4 Hz, 3H), 1.95 (d, J = 9.9 Hz, 2H), 1.83 |
| | | | | (s, 2H), 1.79(m, 1H) 1.56 (s, 2H). |
| 152 | ** | ** | 680 | 1H NMR (400 MHz, DMSO-d6) δ 8.43 (d, J = 6.5 |
| | | | | Hz, 1H), 8.11 (d, J = 7.5 Hz, 1H), 7.33-7.25 (m, |
| | | | | 2H), 7.02 (d, J = 9.2 Hz, 1H), 5.52 (d, J = 10.1 Hz, |
| | | | | 1H), 4.64 (s, 1H), 4.49 (s, 2H), 3.94 (d, J = 11.8 Hz, |
| | | | | 4H), 3.84 (s, 2H), 3.64 (s, 2H), 3.32 (s, 2H), 2.97 (s, |
| | | | | 2H), 2.87 (s, 1H), 2.80 (s, 1H), 2.33-2.32(m, 1H), 2.04 |
| | | | | (s, 2H), 2.00-1.90 (m, 2H), 1.82 (s, 2H), 1.56 (s, |
| | | | | 2H), 1.37 (d, J = 3.3 Hz, 9H). |
| 153 | ** | ** | 496 | 1H NMR (400 MHz, DMSO-d6) δ 8.23 (s, 2H), 7.29 |
| | | | | (ddd, J = 11.8, 9.1, 4.1 Hz, 2H), 7.04-6.96 (m, 1H), |
| | | | | 4.62 (s, 1H), 4.51 (s, 1H), 4.43 (s, 1H), 3.83 (d, J = |
| | | | | 6.7 Hz, 1H), 3.71 (dt, J = 21.0, 7.4 Hz, 1H), 3.68- |
| | | | | 3.60(m, 1H) 3.57 (dd, J = 16.4, 8.3 Hz, 3H), 3.33 (s, |
| | | | | 13H), 2.95 (t, J = 9.4 Hz, 3H), 2.80 (d, J = 22.1 Hz, |
| | | | | 1H), 2.69-2.66 (m, 1H), 2.34-2.31 (m, 1H), 2.04 |
| | | | | (s, 2H), 1.88 (s, 3H). |
| 154 | **** | ** | 471 | 1H NMR (400 MHz, DMSO-d6) δ 8.33 (s, 1H), 8.13 |
| | | | | (s, 1H), 7.29 (ddd, J = 11.7, 9.1, 4.3 Hz, 2H), 7.01 (t, |
| | | | | J = 8.7 Hz, 1H), 4.55 (s, 2H), 4.51 (s, 1H), 3.74 (t, J = |
| | | | | 5.9 Hz, 2H), 3.66 (s, 2H), 3.00-2.91 (m, 2H), 2.83 |
| | | | | (t, J = 5.7 Hz, 2H), 2.04 (s, 2H), 1.80 (d, J = 8.7 Hz, 2H). |
| 155 | ** | ** | 569 | 1H NMR (400 MHz, DMSO-d6) δ 8.43 (s, 1H), 8.11 |
| | | | | (s, 1H), 7.35-7.25 (m, 2H), 7.04-6.97 (m, 1H), |
| | | | | 5.55-5.49 (m, 1H), 4.61-4.47 (m, 3H), 4.09 (q, J = |
| | | | | 7.1 Hz, 2H), 3.94 (d, J = 12.2 Hz, 1H), 3.74 (t, J = 5.6 |
| | | | | Hz, 2H), 3.70-3.62 (m, 1H), 2.97 (dd, J = 12.8, 9.3 |
| | | | | Hz, 2H), 2.83 (t, J = 5.6 Hz, 2H), 2.35-2.31 (m, |
| | | | | 1H), 2.14 (d, J = 11.3 Hz, 2H), 2.04 (s, 2H), 1.95 (d, |
| | | | | J = 10.0 Hz, 2H), 1.82 (d, J = 8.4 Hz, 3H), 1.69 (s, 1H), |
| | | | | 1.56 (s, 2H), 1.22 (t, J = 7.1 Hz, 3H). |
| 156 | ** | ** | 583 | 1H NMR (400 MHz, DMSO-d6) δ 8.43 (s, 1H), 8.11 |
| | | | | (s, 1H), 7.35-7.26 (m, 2H), 7.04-6.96 (m, 1H), |
| | | | | 5.52 (dd, J = 9.9, 2.0 Hz, 1H), 4.83 (dt, J = 12.5, 6.2 |
| | | | | Hz, 1H), 4.57-4.48 (m, 3H), 3.94 (d, J = 11.6 Hz, |
| | | | | 1H), 3.73 (t, J = 5.9 Hz, 2H), 3.70-3.63 (m, 1H), |
| | | | | 2.97 (dd, J = 12.5, 9.1 Hz, 2H), 2.83 (t, J = 5.7 Hz, |
| | | | | 2H), 2.14 (dd, J = 20.8, 9.7 Hz, 1H), 2.04 (s, 2H), |
| | | | | 1.95 (d, J = 10.2 Hz, 2H), 1.82 (d, J = 8.2 Hz, 2H), |
| | | | | 1.74-1.65 (m, 1H), 1.56 (d, J = 3.3 Hz, 2H), 1.23 (s, |
| | | | | 3H), 1.21 (s, 3H). |
| 157 | * | * | 482 | 1H NMR (400 MHz, DMSO-d6) δ 11.87 (s, 1H), 7.62- |
| | | | | 7.56 (m, 1H), 7.51-7.43 (m, 1H), 7.31-7.20 (m, |
| | | | | 2H), 7.02-6.94 (m, 1H), 6.30 (t, J = 6.5 Hz, 1H), |
| | | | | 4.63-4.53 (m, 2H), 4.49-4.39 (m, 1H), 3.83-3.67 |
| | | | | (m, 2H), 3.60-3.47 (m, 2H), 3.08-2.97 (m, 2H), |
| | | | | 2.89 (t, J = 5.7 Hz, 1H), 2.76 (t, J = 5.7 Hz, 1H), 2.15- |
| | | | | 2.07 (m, 3), 1.91-1.81 (m, 2H), 1.59-1.46 (m, 2H). |
| 158 | ** | ** | 482 | 1H NMR (400 MHz, DMSO-d6) δ 10.13 (brs, 1H), |
| | | | | 8.54 (d, J = 1.7 Hz, 1H), 8.15-8.14 (m, 1H), 7.69 |
| | | | | (dd, J = 4.6, 2.7 Hz, 1H), 7.31-7.24 (m, 2H), 7.02- |
| | | | | 6.96 (m, 1H), 4.65 (d, J = 19.6 Hz, 2H), 4.51-4.45 (m, |
| | | | | 1H), 3.82-3.78 (m, 2H), 3.39-3.34 (m, 2H), 2.98- |
| | | | | 2.95 (m, 2H, overlapped), 2.94-2.92 (m, 1H, |
| | | | | overlapped), 2.80 (t, J = 6.0 Hz, 1H), 2.13 (s, 1.68H), |
| | | | | 2.11 (s, 1.42H), 1.93-1.89 (m, 2H), 1.69-1.62 (m, 2H). |
| 159 | ** | ** | 484 | 1H NMR (400 MHz, DMSO-d6) δ 8.98 (s, 1H), 8.73 |
| | | | | (s, 1H), 8.45 (s, 1H), 7.66 (s, 1H), 7.47-7.38 (m, |
| | | | | 1H), 7.21 (td, J = 10.1, 2.5 Hz, 1H), 7.06 (td, J = 8.5, |
| | | | | 2.2 Hz, 1H), 3.91 (s, 3H), 3.52 (bs, 2H), 3.47-3.40 |
| | | | | (m, 4H), 2.42-2.35 (m, 4H). |
| 160 | *** | ** | 514 | 1H NMR (400 MHz, DMSO-d6) δ 8.57 (d, J = 1.6 |
| | | | | Hz, 1H), 8.15 (dd, J = 11.7, 1.8 Hz, 1H), 7.32-7.21 |
| | | | | (m, 2H), 7.03-6.95 (m, 1H), 4.71-4.59 (m, 2H), |
| | | | | 4.54-4.43 (m, 1H), 4.01 (s, 3H), 3.80 (dd, J = 10.0, |
| | | | | 5.4 Hz, 2H), 3.42-3.28 (overlapped m, 2H), 3.04- |
| | | | | 2.89 (m, 3H), 2.80 (t, J = 5.6 Hz, 1H), 2.13 (s, 1.5H), |
| | | | | 2.11 (s, 1.5H), 2.01-1.90 (m, 2H), 1.72-1.58 (m, 2H). |
| 161 | ** | ** | 510 | 1H NMR (400 MHz, DMSO-d6) δ 7.93 (td, J = 7.8, |
| | | | | 1.3 Hz, 1H), 7.72 (d, J = 7.8 Hz, 1H), 7.41 (d, J = 7.7 |
| | | | | H, 1 Hz), 7.33-7.16 (m, 2H), 7.03-6.91 (m, 1H), |
| | | | | 4.65 (s, 1H), 4.59 (s, 1H), 4.53 (s, 2H), 4.50-4.39 |
| | | | | (m, 1H), 3.80 (q, J = 5.7 Hz, 2H), 3.44-3.40 |
| | | | | (overlapping m, 2H), 3.39 (overlapping s, 3H), 3.04- |
| | | | | 2.93 (m, 2H), 2.92 (t, J = 5.8 Hz, 1H), 2.79 (t, J = 5.8 |
| | | | | Hz, 1H), 2.13 (s, 1.6H), 2.10 (s, 1.4H), 1.94-1.76 |
| | | | | (m, 2H), 1.64-1.41 (m, 2H). |
| 162 | *** | ** | 514 | 1H-NMR (400 MHz, DMSO-d6) δ 8.42 (d, J = 2.0 |
| | | | | Hz, 1H), 8.06 (d, J = 3.0 Hz, 1H), 7.36-7.25 (m, |
| | | | | 2H), 7.06-6.96 (m, 1H), 4.68-4.58 (m, 4H), 4.56- |
| | | | | 4.49 (overlapping signals, 3H), 4.45 (t, J = 6.1 Hz, |
| | | | | 2H), 3.83-3.75 (m, 2H), 3.49-3.39 (m, 1H), 3.33- |
| | | | | 3.26 (overlapping signals, 2H), 3.00-2.87 (m, 3H), |
| | | | | 2.77 (t, J = 5.9 Hz, 1H), 2.13 (s, 1.6H), 2.12 (s, 1.4 |
| | | | | H), 2.10-2.02 (m, 2H), 1.91-1.78 (m, 2H). |
| 163 | ** | ** | 552 | 1H NMR (400 MHz, DMSO-d6) δ 8.51-8.48 (m, |
| | | | | 1H), 8.21 (d, J = 2.6 Hz, 1H), 7.34-7.25 (m, 2H), |
| | | | | 7.05-6.95 (m, 1H), 5.45-5.36 (m, 1H), 4.67-4.55 |
| | | | | (m, 3H), 4.55-4.40 (m, 2H), 4.30 (t, J = 9.2 Hz, 1H), |
| | | | | 4.17-4.08 (m, 1H), 3.84-3.72 (m, 2H), 3.33 (m, |
| | | | | 1H, overlapped), 3.31-3.28 (m, 1H, overlapped), |
| | | | | 3.02-2.87 (m, 3H), 2.78 (t, J = 5.7 Hz, 1H), 2.12 (s, |
| | | | | 1.5H, overlapped), 2.11 (s, 1.5H, overlapped), 2.10- |
| | | | | 2.02 (m, 2H, overlapped), 1.89-1.77 (m, 2H, |
| | | | | overlapped), 1.83 (s, 3H, overlapped). |
| 164 | *** | ** | 494 | 1H-NMR (400 MHz, DMSO-d6) δ 8.36 (s, 1H), 8.04 |
| | | | | (d, J = 2.4 Hz, 1H), 7.35-7.23 (m, 2H), 7.06-6.96 |
| | | | | (m, 1H), 4.64 (s, 1H), 4.60 (s, 1H), 4.55-4.46 (m, |
| | | | | 1H), 3.90-3.82 (m, 1H), 3.79 (dd, J = 9.3, 5.6 Hz, |
| | | | | 2H), 3.31 (m, 2H), 2.95 (t, J = 9.7 Hz, 2H), 2.89 (t, |
| | | | | J = 5.8 Hz, 1H), 2.76 (t, J = 5.7 Hz, 1H), 2.12 (s, 1.6H), |
| | | | | 2.11 (s, 1.4H), 2.09-2.00 (m, 2H), 1.88-1.72 (m, |
| | | | | 2H), 1.14-1.06 (m, 2H), 1.04-0.94 (m, 2H). |
| 165 | **** | ** | 497 | 1H-NMR (400 MHz, DMSO-d6) δ 8.35 (s, 1H), 8.06 |
| | | | | (s, 1H), 7.34-7.22 (m, 2H), 7.05-6.95 (m, 1H), |
| | | | | 4.68-4.57 (overlapping m, 3H), 4.55-4.44 (m, 1H), |
| | | | | 3.83-3.73 (m, 2H), 3.32-3.27 (m, 2H), 2.96 (t, J = |
| | | | | 10.3 Hz, 2H), 2.90 (t, J = 5.6 Hz, 1H), 2.76 (t, J = 5.5 |
| | | | | Hz, 1H), 2.13 (s, 1.6H) 2.11 (s, 1.4H), 2.10-2.01 (m, |
| | | | | 2H), 1.87-1.77 (m, 2H), 1.46 (d, J = 6.7 Hz, 6H). |
| 166 | *** | ** | 532 | 1HNMR (400 MHz, DMSO-d6) δ 8.29 (bs, 1H), 8.04 |
| | | | | (bs, 1H), 7.88-7.82 (m, 1H), 7.66 (d, J = 7.9 Hz, |
| | | | | 1H), 7.54 (d, J = 7.7 Hz, 1H), 7.24-7.12 (m, 2H), |
| | | | | 6.94-6.86 (m, 1H), 4.62 (s, 1H), 4.56 (s, 1H), 4.45- |
| | | | | 4.33 (m, 1H), 3.77 (q, J = 5.6 Hz, 2H), 3.50-3.37 |
| | | | | (m, 2H), 3.06-2.94 (m, 2H), 2.89 (t, J = 5.7 Hz, 1H), |
| | | | | 2.76 (t, J = 5.8 Hz, 1H), 2.10 (s, 1.7H), 2.07 (s, 1.3H), |
| | | | | 1.86-1.74 (m, 2H), 1.58-1.46 (m, 2H). |
| 167 | ** | ** | 482 | 1H NMR (400 MHz, DMSO-d6) δ 11.47 (s, 1H), 7.59 |
| | | | | (t, J = 7.2, 8.8 Hz 1H), 7.32-7.22 (m, 2H), 7.01 (t, J = |
| | | | | 7.9 Hz, 1H), 6.78 (s, 1H), 6.41 (d, J = 9.3 Hz, 1H), |
| | | | | 4.65 (d, J = 17.0 Hz, 2H), 4.52 (d, J = 3.6 Hz, 1H), |
| | | | | 3.83 (t, J = 5.8 Hz, 2H), 3.42 (br s, 2H), 3.07-3.01 (m, |
| | | | | 2H), 2.95 (t, J = 5.5 Hz, 1H), 2.83 (d, J = 5.7 Hz, 1H), |
| | | | | 2.14 (d, J = 5.8 Hz, 3H), 1.98 (s, 2H), 1.78-1.70 (m, 2H). |
| 168 | **** | *** | 481 | 1H NMR (400 MHz, DMSO-d6) δ 7.48 (p, J = 5.7 |
| | | | | Hz, 1H), 7.25 (ddd, J = 14.8, 9.1, 4.2 Hz, 2H), 6.98 |
| | | | | (ddd, J = 11.2, 3.9, 2.4 Hz, 1H), 6.86 (dd, J = 7.2, 2.9 |
| | | | | Hz, 1H), 6.43 (d, J = 8.2 Hz, 1H), 6.04 (s, 2H), 4.58 |
| | | | | (d, J = 18.2 Hz, 2H), 4.48-4.41 (m, 1H), 3.82-3.73 |
| | | | | (m, 2H), 3.45 (d, J = 8.9 Hz, 2H), 3.04-2.94 (m, |
| | | | | 2H), 2.89 (t, J = 5.8 Hz, 1H), 2.76 (t, J = 5.7 Hz, 1H), |
| | | | | 2.11 (d, J = 11.2 Hz, 3H), 1.86 (s, 2H), 1.64-1.53 |
| | | | | (m, 2H). |
| 169 | ** | ** | 511 | 1H NMR (400 MHz, DMSO-d6) δ 8.39 (s, 1H), 8.09 |
| | | | | (s, 1H), 7.35-7.25 (m, 2H), 7.05-6.95 (m, 1H), |
| | | | | 5.51 (dd, J = 9.8, 2.0 Hz, 1H), 4.55-4.47 (m, 1H), |
| | | | | 3.94 (d, J = 11.4 Hz, 1H), 3.70-3.62 (m, 1H), 3.52 |
| | | | | (s, 2H), 3.32 (s, 2H), 2.95 (dd, J = 12.4, 9.2 Hz, 2H), |
| | | | | 2.84 (t, J = 5.8 Hz, 2H), 2.73-2.65 (m, 2H), 2.39- |
| | | | | 2.32(s, 3H), 2.18-2.10 (m, 1H), 2.04 (s, 2H), 1.95 (d, |
| | | | | J = 10.1 Hz, 2H), 1.87-1.74 (m, 2H), 1.74-1.60 (m, |
| | | | | 1H), 1.56 (d, J = 3.6 Hz, 2H). |
| 170 | *** | ** | 441 | 1H NMR (400 MHz, DMSO-d6) δ 13.05 (s, 1H), 8.41- |
| | | | | 8.00 (m, 2H), 7.29 (ddd, J = 11.8, 9.2, 4.2 Hz, 2H), |
| | | | | 7.06-6.93 (m, 1H), 4.56-4.46 (m, 1H), 3.56 (s, |
| | | | | 2H), 3.32 (s, 2H), 2.93 (t, J = 9.5 Hz, 2H), 2.81 (d, J = |
| | | | | 5.3 Hz, 2H), 2.76 (d, J = 5.1 Hz, 2H), 2.62-2.53 (m, |
| | | | | 2H), 2.04 (s, 2H), 1.88-1.74 (m, 2H), 1.11 (t, J = 7.1 |
| | | | | Hz, 3H). |
| 171 | **** | ** | 438 | 1H NMR (400 MHz, DMSO-d6) δ 13.12 (s, 1H), 8.21 |
| | | | | (s, 2H), 7.33-7.24 (m, 2H), 7.04-6.97 (m, 1H), |
| | | | | 4.56-4.48 (m, 1H), 4.43 (s, 2H), 3.60 (t, J = 5.9 Hz, |
| | | | | 2H), 3.32 (s, 2H), 3.04-2.91 (m, 4H), 2.07 (t, J = 9.3 |
| | | | | Hz, 3H), 1.87-1.68 (m, 2H). |
| 172 | *** | ** | 581 | 1H NMR (400 MHz, DMSO-d6) δ 8.42 (d, J = 8.3 |
| | | | | Hz, 1H), 8.11 (d, J = 10.3 Hz, 1H), 7.29 (ddd, J = |
| | | | | 11.7, 9.1, 4.2 Hz, 2H), 7.01 (ddd, J = 11.1, 3.9, 2.4 |
| | | | | Hz, 1H), 5.52 (d, J = 8.0 Hz, 1H), 4.79-4.64 (m, |
| | | | | 5H), 4.51 (s, 1H), 4.39 (s, 1H), 4.27 (dt, J = 15.4, 7.7 |
| | | | | Hz, 1H), 3.94 (d, J = 11.4 Hz, 1H), 3.85 (t, J = 6.0 Hz, |
| | | | | 1H), 3.70-3.62 (m, 1H), 3.54 (t, J = 5.9 Hz, 1H), |
| | | | | 3.02-2.87 (m, 2H), 2.82 (dt, J = 16.5, 5.7 Hz, 2H), |
| | | | | 2.18-2.10 (m, 1H), 2.10-2.00 (m, 2H), 1.96 (d, J = |
| | | | | 10.1 Hz, 2H), 1.81 (d, J = 9.5 Hz, 2H), 1.75-1.63 |
| | | | | (m, 1H), 1.56 (d, J = 3.6 Hz, 2H). |
| 173 | ** | ** | 597 | 1H NMR (400 MHz, DMSO-d6) δ 8.44 (s, 1H), 8.12 |
| | | | | (s, 1H), 7.36-7.25 (m, 2H), 7.05-6.96 (m, 1H), |
| | | | | 5.52 (dd, J = 9.9, 1.9 Hz, 1H), 5.10 (s, 1H), 4.66 (s, |
| | | | | 1H), 4.51 (dd, J = 7.6, 3.9 Hz, 1H), 4.23 (s, 1H), 3.94 |
| | | | | (d, J = 11.6 Hz, 1H), 3.88 (s, 1H), 3.71-3.61 (m, |
| | | | | 1H), 3.19 (s, 1H), 3.05 (s, 1H), 2.97 (dd, J = 11.8, 9.5 |
| | | | | Hz, 2H), 2.93-2.87 (m, 1H), 2.81 (s, 1H), 2.14 (dt, |
| | | | | J = 13.3, 6.5 Hz, 1H), 2.06 (d, J = 12.8 Hz, 2H), 1.95 |
| | | | | (d, J = 10.3 Hz, 2H), 1.82 (d, J = 8.6 Hz, 2H), 1.75- |
| | | | | 1.60 (m, 1H), 1.56 (d, J = 3.5 Hz, 2H), 1.34 (s, 6H). |
| 174 | *** | ** | 513 | 1H NMR (400 MHz, DMSO-d6) δ 13.09 (s, 1H), 8.37 |
| | | | | (d, J = 21.4 Hz, 1H), 8.11 (d, J = 14.1 Hz, 1H), 7.29 |
| | | | | (ddd, J = 11.8, 9.0, 4.3 Hz, 2H), 7.01 (t, J = 8.6 Hz, |
| | | | | 1H), 5.52 (dd, J = 9.9, 1.9 Hz, 1H), 5.09 (s, 1H), 4.65 |
| | | | | (s, 1H), 4.53 (d, J = 15.4 Hz, 1H), 4.22 (s, 1H), 3.85 |
| | | | | (d, J = 17.4 Hz, 1H), 3.57 (s, 1H), 3.19 (s, 2H), 3.05 |
| | | | | (s, 1H), 3.00-2.88 (m, 3H), 2.80 (s, 1H), 2.06 (d, J = |
| | | | | 17.7 Hz, 2H), 1.98 (d, J = 6.5 Hz, 1H), 1.94 (s, 1H), |
| | | | | 1.80 (d, J = 8.9 Hz, 2H), 1.34 (s, 6H), 1.24 (s, 2H) |
| 175 | *** | ** | 499 | 1H NMR (400 MHz, DMSO-d6) δ 13.09 (s, 1H), 8.24 |
| | | | | (d, J = 83.0 Hz, 2H), 7.35-7.25 (m, 2H), 7.10-6.94 |
| | | | | (m, 2H), 5.53 (s, 1H), 5.15 (s, 1H), 4.62 (s, 1H), 4.51 |
| | | | | (d, J = 3.7 Hz, 1H), 4.25 (s, 1H), 3.83 (s, 1H), 2.99- |
| | | | | 2.91 (m, 2H), 2.85 (s, 2H), 2.04 (s, 2H), 1.80 (dd, J = |
| | | | | 11.0, 6.5 Hz, 2H), 1.29 (d, J = 44.3 Hz, 6H). |
| 176 | *** | ** | 603 | 1H NMR (400 MHz, DMSO-d6) δ 8.42 (s, 1H), 8.11 |
| | | | | (s, 1H), 7.36-7.22 (m, 2H), 7.05-6.95 (m, 1H), |
| | | | | 5.52 (dd, J = 9.8, 2.0 Hz, 1H), 4.51 (dd, J = 11.9, 8.2 |
| | | | | Hz, 1H), 4.46 (s, 2H), 3.94 (d, J = 11.7 Hz, 1H), 3.66 |
| | | | | (dt, J = 13.5, 6.9 Hz, 3H), 3.50 (dt, J = 13.5, 6.8 Hz, |
| | | | | 1H), 3.32 (s, 2H), 2.98 (dd, J = 12.5, 9.2 Hz, 2H), |
| | | | | 2.89 (t, J = 5.8 Hz, 2H), 2.19-2.11 (m, 1H), 2.05 |
| | | | | (dd, J = 23.4, 12.3 Hz, 2H), 1.95 (d, J = 10.3 Hz, 2H), |
| | | | | 1.82 (d, J = 11.9 Hz, 2H), 1.76-1.61 (m, 2H), 1.56 |
| | | | | (d, J = 3.6 Hz, 2H), 1.26 (d, J = 6.8 Hz, 6H). |
| 177 | *** | ** | 589 | 1H NMR (400 MHz, DMSO-d6) δ 8.42 (s, 1H), 8.11 |
| | | | | (s, 1H), 7.35-7.25 (m, 2H), 7.04-6.96 (m, 1H), |
| | | | | 5.52 (dd, J = 9.8, 2.0 Hz, 1H), 4.56-4.47 (m, 1H), |
| | | | | 4.43 (s, 2H), 3.94 (d, J = 11.6 Hz, 1H), 3.70-3.59 |
| | | | | (m, 3H), 3.32 (s, 1H), 3.21 (q, J = 7.3 Hz, 2H), 2.98 |
| | | | | (dd, J = 12.6, 9.3 Hz, 2H), 2.91 (t, J = 5.8 Hz, 2H), |
| | | | | 2.14 (dd, J = 9.2, 2.2 Hz, 1H), 2.10-2.00 (m, 2H), |
| | | | | 1.97 (t, J = 9.2 Hz, 2H), 1.88-1.76 (m, 2H), 1.75- |
| | | | | 1.62 (m, 1H), 1.56 (d, J = 3.5 Hz, 2H), 1.23 (t, J = 7.3 |
| | | | | Hz, 3H). |
| 178 | *** | ** | 510 | 1H NMR (400 MHz, DMSO-d6) δ 13.10 (s, 1H), 8.23 |
| | | | | (s, 2H), 7.38-7.23 (m, 2H), 7.06-6.95 (m, 1H), |
| | | | | 4.82-4.57 (m, 2H), 4.56-4.46 (m, 1H), 3.89-3.74 |
| | | | | (m, 3H), 3.04-2.85 (m, 5H), 2.79 (s, 1H), 2.69- |
| | | | | 2.59 (m, 2H), 2.04 (s, 3H), 1.81 (d, J = 9.0 Hz, 2H), |
| | | | | 1.63 (ddd, J = 18.0, 11.3, 4.7 Hz, 3H). |
| 179 | **** | ** | 471 | 1H NMR (400 MHz, DMSO-d6) δ 13.11 (s, 1H), 8.39- |
| | | | | 8.01 (m, 3H), 7.29 (ddd, J = 11.7, 9.0, 4.3 Hz, 2H), |
| | | | | 7.00 (ddd, J = 4.7, 3.8, 1.7 Hz, 1H), 4.76-4.67 (m, |
| | | | | 1H), 4.61 (d, J = 21.3 Hz, 2H), 4.55-4.47 (m, 1H), |
| | | | | 4.22 (dd, J = 11.7, 5.0 Hz, 2H), 3.84 (s, 1H), 3.72 (s, |
| | | | | 1H), 3.32 (s, 25H), 2.94 (dd, J = 21.4, 11.9 Hz, 3H), |
| | | | | 2.80 (s, 1H), 2.06 (d, J = 13.3 Hz, 2H), 1.87-1.74 |
| | | | | (m, 2H). |
| 180 | ** | ** | 470 | 1H NMR (400 MHz, DMSO-d6) δ 13.09 (s, 1H), 8.32 |
| | | | | (s, 1H), 8.10(s, 1H) 7.35-7.24 (m, 2H), 7.01 (t, J = 8.6 |
| | | | | Hz, 1H), 6.67 (d, J = 4.3 Hz, 1H), 4.49 (s, 3H), 3.67 |
| | | | | (t, J = 5.5 Hz, 2H), 3.03-2.88 (m, 2H), 2.78 (s, 2H), |
| | | | | 2.60 (d, J = 4.2 Hz, 3H), 2.05 (s, 2H), 1.80 (d, J = 9.0 |
| | | | | Hz, 2H). |
| 181 | ** | ** | 554 | 1H NMR (400 MHz, DMSO-d6) δ 8.40 |
| | | | | (s, 1H), 8.09(s, 1H) 7.35-7.24 (m, 2H), 7.01 (t, J = 8.6 |
| | | | | Hz, 1H), 6.67 (d, J = 4.3 Hz, 1H), 5.54- |
| | | | | 5.50(d, j = 2.56 HZ, 1H), 4.49 (s, 3H), 3.95(d, j = 16 |
| | | | | HZ, 1H), 3.67 (d, J = 4.8 Hz, 2H), 2.99-2.92(m, 2H), |
| | | | | 2.80-2.76 (m, 2H), 2.60 (d, J = 4.2 Hz, 3H), 2.11-2.07 |
| | | | | (m, 3H), 1.97 (d, J = 14 Hz, 2H), 1.82-1.71(m, |
| | | | | 3H), 1.69(s, 2H). |
| 182 | *** | ** | 568 | 1H NMR (400 MHz, DMSO-d6) δ 8.42 (s, 1H), 8.11 |
| | | | | (s, 1H), 7.36-7.23 (m, 2H), 7.06-6.93 (m, 1H), |
| | | | | 5.52 (dd, J = 9.8, 2.0 Hz, 1H), 4.51 (dd, J = 7.8, 3.9 |
| | | | | Hz, 1H), 4.32 (s, 2H), 3.94 (d, J = 11.6 Hz, 1H), 3.73- |
| | | | | 3.60 (m, 1H), 3.50 (t, J = 5.7 Hz, 2H), 2.96 (dd, J = |
| | | | | 12.5, 9.1 Hz, 2H), 2.87 (t, J = 5.6 Hz, 2H), 2.80 (s, |
| | | | | 6H), 2.20-2.00 (m, 3H), 1.95 (d, J = 10.2 Hz, 2H), |
| | | | | 1.82 (d, J = 9.0 Hz, 2H), 1.68 (dd, J = 16.4, 10.7 Hz, |
| | | | | 1H), 1.56 (d, J = 3.4 Hz, 2H). |
| 183 | **** | ** | 527 | 1H NMR (400 MHz, DMSO-d6) δ 7.86-7.80 (m, |
| | | | | 1H), 7.51 (d, J = 2.6 Hz, 1H), 7.46 (d, J = 8.5 Hz, |
| | | | | 1H), 7.36 (d, J = 8.4 Hz, 2H), 6.82 (d, J = 8.4 Hz, |
| | | | | 1H), 4.64 (s, 1H), 4.59 (s, 1H), 3.87 (s, 3H), 3.80 (d, |
| | | | | J = 5.4 Hz, 2H), 3.55 (s, 2H), 3.14 (s, 4H), 2.91 (s, 1H), |
| | | | | 2.78 (s, 1H), 2.34-2.31 (m, 4H), 2.12 (d, J = 11.4 |
| | | | | Hz, 3H). |
| 184 | **** | ** | 497 | 1H NMR (400 MHz, DMSO-d6) δ 13.10 (s, 1H), 8.35 |
| | | | | (s, 1H), 8.12 (s, 1H), 7.35-7.25 (m, 2H), 7.05-6.96 |
| | | | | (m, 1H), 4.79-4.64 (m, 5H), 4.51 (s, 1H), 4.39 (s, |
| | | | | 1H), 4.27 (dt, J = 15.5, 7.8 Hz, 1H), 3.85 (t, J = 5.9 |
| | | | | Hz, 1H), 3.54 (t, J = 5.8 Hz, 1H), 3.32 (s, 13H), 2.95 |
| | | | | (t, J = 9.5 Hz, 2H), 2.81 (dt, J = 16.3, 5.6 Hz, 2H), |
| | | | | 2.06 (d, J = 14.0 Hz, 2H), 1.80 (d, J = 8.9 Hz, 2H). |
| 185 | *** | ** | 551 | 1H NMR (400 MHz, DMSO-d6) δ 8.43 (s, 1H), 8.11 |
| | | | | (d, J = 7.3 Hz, 1H), 7.29 (ddd, J = 14.3, 6.9, 4.4 Hz, |
| | | | | 2H), 7.06-6.86 (m, 2H), 6.21-6.07 (m, 1H), 5.75 |
| | | | | (d, J = 10.7 Hz, 1H), 5.52 (d, J = 8.1 Hz, 1H), 4.74 (d, |
| | | | | J = 31.1 Hz, 2H), 4.52 (s, 1H), 3.91 (d, J = 16.1 Hz, |
| | | | | 3H), 3.72-3.61 (m, 1H), 2.96 (d, J = 12.6 Hz, 2H), |
| | | | | 2.86 (d, J = 25.6 Hz, 2H), 2.13 (s, 1H), 2.06 (d, J = |
| | | | | 16.5 Hz, 2H), 1.96 (d, J = 10.2 Hz, 2H), 1.81 (d, J = |
| | | | | 7.0 Hz, 2H), 1.69 (s, 1H), 1.56 (s, 2H). |
| 186 | ** | ** | 694 | 1H NMR (400 MHz, DMSO-d6) δ 8.42 (d, j = 6, 1H), |
| | | | | 8.12 (d, j = 4, 1H), 7.38-7.23 (m, 2H), 7.06-6.95 (m, |
| | | | | 1H), 5.53(d, j = 12, 1H), (4.89-4.36 (m, 4H), 4.56- |
| | | | | 4.46 (m, 1H), 3.96 (d, j = 14.4, 2H), 3.70-3.61(m, |
| | | | | 2H), 3.4(3.3 (s, 4H), 2.97-2.80 (m, 4H), 2.08-1.93 |
| | | | | (m, 6H), 1.80 (s, 6H), 1.55 (s, 2H), 1.37(S, 9H). |
| 187 | **** | ** | 484 | 1H NMR (400 MHz, DMSO-d6) δ 13.09 (s, 1H), |
| | | | | 8.23-8.02 (m, 2H), 7.39-7.21 (m, 2H), 7.07-6.95 |
| | | | | (m, 1H), 4.50 (m, 1H), 4.31 (s, 2H), 3.49 (t, J = 5.7 |
| | | | | Hz, 2H), 3.00-2.90 (m, 2H), 2.86 (t, J = 5.5 Hz, 2H), |
| | | | | 2.80 (s, 6H), 2.04 (bs, 2H), 1.87-1.72 (m, 2H). |
| 188 | *** | ** | 435 | 1H-NMR (400 MHz, DMSO-d6) δ 9.10 (s, 1H), 8.54 |
| | | | | (d, J = 5.7 Hz, 1H), 8.50 (s, 1H), 8.17 (s, 1H), 7.61 (d, |
| | | | | J = 5.7 Hz, 1H), 7.35-7.20 (m, 2H), 7.06-6.93 (m, |
| | | | | 1H), 3.95 (s, 3H), 3.65-3.55 (m, 2H), 3.50-3.44 |
| | | | | (overlapping m, 2H), 2.01-1.92 (m, 2H), 1.89-1.79 |
| | | | | (m, 2H), 1.24 (s, 3H). |
| 189 | *** | ** | 497 | 1H NMR (400 MHz, DMSO-d6) δ 8.01 (dd, J = 9.2, |
| | | | | 2.3 Hz, 1H), 7.35 (dd, J = 9.2, 2.8 Hz, 1H), 7.31- |
| | | | | 7.20 (m, 2H), 7.02-6.93 (m, 1H), 4.66 (s, 1H), 4.62 |
| | | | | (s, 1H), 4.53-4.42 (m, 1H), 4.08 (s, 3H), 3.87-3.77 |
| | | | | (m, 2H), 3.47-3.36 (m, 2H), 3.08-2.97 (m, 2H), |
| | | | | 2.95 (t, J = 5.8 H, 1 Hz), 2.82 (t, J = 5.7 Hz, 1H), 2.14 |
| | | | | (s, 1.7H), 2.11 (s, 1.3H), 1.92-1.80 (m, 2H), 1.67- |
| | | | | 1.48 (m, 2H). |
| 190 | *** | ** | 497 | 1H NMR (400 MHz, DMSO-d6) δ 9.12 (overalpping |
| | | | | s, 1H), 9.12 (overlapping s, 1H), 7.33-7.21 (m, 2H), |
| | | | | 7.04-6.94 (m, 1H), 4.68 (s, 1H), 4.64 (s, 1H), 4.55- |
| | | | | 4.42 (m, 1H), 3.98 (s, 3H), 3.80 (dd, J = 9.4, 5.6 Hz, |
| | | | | 2H), 3.38-3.29 (m, 2H), 3.07-2.88 (m, 3H), 2.81 (t, |
| | | | | J = 5.7 Hz, 1H), 2.13 (s, 1.6H), 2.11 (s, 1H), 2.04- |
| | | | | 1.82 (m, 2H), 1.75-1.57 (m, 2H). |
| 191 | ** | ** | 497 | 1H NMR (400 MHz, DMSO-d6) δ 8.69 (s, 2H), 7.32- |
| | | | | 7.19 (m, 2H), 7.02-6.92 (m, 1H), 4.62 (s, 1H), |
| | | | | 4.57 (s, 1H), 4.50-4.40 (m, 1H), 3.97 (s, 3H), 3.80 |
| | | | | (q, J = 5.7 Hz, 2H), 3.43-3.36 (overlapping m, 2H), |
| | | | | 3.06-2.90 (m, 3H), 2.80 (t, J = 5.8 Hz, 1H), 2.13 (s, |
| | | | | 1.7H), 2.10 (s, 1.3H), 1.91-1.78 (m, 2H), 1.59-1.45 |
| | | | | (m, 2H). |
| 192 | ** | ** | 497 | 1H NMR (400 MHz, DMSO-d6) δ 9.02 (t, J = 3.0 Hz, |
| | | | | 1H), 7.59 (dd, J = 5.2, 3.0 Hz, 1H), 7.33-7.17 (m, |
| | | | | 2H), 7.03-6.88 (m, 1H), 4.67 (s, 1H), 4.63 (s, 1H), |
| | | | | 4.56-4.41 (m, 1H), 3.98 (s, 3H), 3.87-3.76 (m, |
| | | | | 2H), 3.51-3.41 (m, 2H), 3.11-2.99 (m, 2H), 2.96 (t, |
| | | | | J = 5.7 Hz, 1H), 2.83 (t, J = 5.8 Hz, 1H), 2.14 (s, |
| | | | | 1.7H), 2.11 (s, 1.3H), 1.91-1.78 (m, 2H), 1.61-1.49 |
| | | | | (m, 2H) |
| 193 | ** | ** | 456 | 1H NMR (400 MHz, DMSO-d6) δ 8.91 (s, 1H), 8.43 |
| | | | | (s, 1H), 8.09 (s, 1H), 7.68 (d, J = 0.7 Hz, 1H), 7.51- |
| | | | | 7.42 (m, 1H), 7.21 (td, J = 10.0, 2.4 Hz, 1H), 7.07 (td, |
| | | | | J = 8.4, 2.2 Hz, 1H), 3.94 (s, 3H), 3.56 (s, 2H), 3.48- |
| | | | | 3.37 (m, 2H), 2.59-2.52 (overlapped m, 4H). |
| 194 | *** | ** | 452 | 1H NMR (400 MHz, DMSO-d6) δ 9.03 (s, 1H), 8.44 |
| | | | | (s, 1H), 8.12 (s, 1H), 7.61 (s, 1H), 7.52-7.44 (m, |
| | | | | 1H), 7.22 (td, J = 10.1, 2.5 Hz, 1H), 7.08 (td, J = 8.4, |
| | | | | 2.1 Hz, 1H), 5.54 (bs, 1H), 4.69 (s, 2H), 3.94 (s, 3H), |
| | | | | 3.57 (s, 2H), 3.44-3.22 (overlapped m, 4H), 2.63- |
| | | | | 2.53 (overlapped m, 4H). |
| 195 | *** | ** | 511 | 1H NMR (400 MHz, DMSO-d6) δ 8.34 (s, 1H), 8.07- |
| | | | | 8.03 (m, 1H), 7.35-7.24 (m, 2H), 7.06-6.96 (m, |
| | | | | 1H), 4.62 (d, J = 17.1 Hz, 2H), 4.55-4.45 (m, 1H), |
| | | | | 4.05-3.95 (m, 2H), 3.83-3.75 (m, 2H), 3.32-3.28 |
| | | | | (m, 2H, overlapping), 3.01-2.86 (m, 3H), 2.76 (t, J = |
| | | | | 5.8 Hz, 1H), 2.20-2.14 (m, 1H, overlapping), 2.13 |
| | | | | (s, 1.5H, overlapping), 2.11 (s, 1.5H, overlapping), |
| | | | | 2.10-2.01 (m, 2H, overlapping), 1.90-1.75 (m, |
| | | | | 2H), 0.85 (d, J = 6.7 Hz, 6H). |
| 196 | ** | ** | 522 | 1H NMR (400 MHz, DMSO-d6) δ 8.53 (s, 1H), 8.28- |
| | | | | 8.20 (m, 1H), 7.38-7.17 (m, 2H), 7.08-6.91 (m, |
| | | | | 1H), 4.65 (d, J = 14.1 Hz, 2H), 4.56-4.46 (m, 1H), |
| | | | | 3.83-3.74 (m, 2H), 3.42-3.24 (m, 2H, |
| | | | | overlapping), 2.99 (t, J = 10.4 Hz, 2H), 2.92 (t, J = 5.5 |
| | | | | Hz, 1H), 2.79 (t, J = 5.6 Hz, 1H), 2.13 (s, 1.5H, |
| | | | | overlapping), 2.11 (s, 1.5H, overlapping), 2.10-2.05 |
| | | | | (m, 2H, overlapping), 2.04 (s, 6H, overlapping), 1.88- |
| | | | | 1.74 (m, 2H). |
| 197 | *** | ** | 510 | 1H NMR (400 MHz, DMSO-d6) δ 9.02 (s, 1H), 8.45 |
| | | | | (s, 1H), 8.12 (s, 1H), 7.69 (s, 1H), 7.52-7.44 (m, |
| | | | | 1H), 7.22 (td, J = 10.0, 2.5 Hz, 1H), 7.08 (td, J = 8.5, |
| | | | | 2.2 Hz, 1H), 5.49 (d, J = 4.8 Hz, 1H), 4.76 (s, 1H), |
| | | | | 4.51 (d, J = 3.9 Hz, 1H), 3.94 (s, 3H), 3.58 (s, 2H), |
| | | | | 3.43-3.25 (overlapped m, 4H), 2.62-2.53 |
| | | | | (overlapped m, 4H), 1.07 (s, 3H), 1.05 (s, 3H). |
| 198 | *** | ** | 471 | 1H NMR (400 MHz, DMSO-d6) δ 7.38-7.19 (m, |
| | | | | 2H), 7.15-6.89 (m, 1H), 6.53 (d, J = 1.5 Hz, 1H), |
| | | | | 4.61-4.45 (overlapping m, 3H), 4.25 (d, J = 1.5 Hz, |
| | | | | 2H), 3.85-3.73 (m, 4H), 3.60-3.47 (m, 2H), 3.33 |
| | | | | (overlapping m, 1H) 3.13-3.00 (m, 2H), 2.92-2.79 |
| | | | | (m, 1H), 2.79-2.66 (m, 1H), 2.51-2.54 (overlapping |
| | | | | m, 1 H), 2.09 (d, J = 7.9 Hz, 3H), 2.06-1.94 (m, 2H), |
| | | | | 1.79-1.63 (m, 2H). |
| 199 | *** | ** | 469 | 1H NMR (400 MHz, DMSO-d6) δ 8.28 (s, 1H), 7.20- |
| | | | | 7.12 (m, 1H), 7.01-6.93 (m, 1H), 6.89-6.82 (m, 1H), |
| | | | | 4.85-4.77 (m, 2H), 4.72 (t, J = 16.4 Hz, 2H), 4.46- |
| | | | | 4.43 (m, 1H), 3.81 (t, , J = 16.8 Hz 1H), 3.57 (s, 2H), |
| | | | | 3.46-3.39 (m, 2H), 3.07-2.94 (m, 4H), 2.77 (t, J = 8 |
| | | | | Hz, 2H), 2.12-2.06 (m, 2H), 1.92-1.86 (m, 2H). |
| 200 | ** | ** | 522 | 1H NMR (400 MHz, DMSO-d6) δ 8.42 (s, 1H), 8.10 |
| | | | | (s, 1H), 7.34-7.25 (m, 2H), 7.05-6.98 (m, 1H), |
| | | | | 5.53 (dd, J = 9.7, 1.8 Hz, 1H), 4.56-4.49 (m, 1H), |
| | | | | 4.44 (s, 2H), 3.95 (d, J = 11.7 Hz, 1H), 3.71-3.64 |
| | | | | (m, 1H), 3.61 (dd, J = 10.8, 5.0 Hz, 2H), 2.98 (dt, J = |
| | | | | 11.5, 7.5 Hz, 4H), 2.17-2.10 (m, 1H), 2.05 (t, J = |
| | | | | 19.7 Hz, 2H), 1.96 (d, J = 10.1 Hz, 2H), 1.82 (d, J = |
| | | | | 9.1 Hz, 2H), 1.75-1.61 (m, 1H), 1.57 (d, J = 3.5 Hz, 2H). |
| 201 | ** | ** | 569 | 1H NMR (400 MHz, DMSO-d6) δ 8.43 (s, 1H), 8.12 |
| | | | | (s, 1H), 7.35-7.23 (m, 2H), 7.05-6.96 (m, 1H), |
| | | | | 5.52 (dd, J = 9.8, 1.8 Hz, 1H), 4.62 (s, 2H), 4.55- |
| | | | | 4.49 (m, 1H), 4.22 (d, J = 6.4 Hz, 2H), 3.94 (d, J = |
| | | | | 11.8 Hz, 1H), 3.82 (s, 1H), 3.75 (s, 1H), 3.70-3.61 |
| | | | | (m, 1H), 3.32(d, J = 9.6, 5H), 2.97 (dd, J = 12.3, 9.3 Hz, |
| | | | | 2H), 2.90 (d, J = 5.1 Hz, 1H), 2.80 (s, 1H), 2.10 (dd, |
| | | | | J = 32.8, 11.5 Hz, 3H), 1.96 (d, J = 10.1 Hz, 2H), 1.82 |
| | | | | (dd, J = 14.7, 5.8 Hz, 2H), 1.75-1.60 (m, 1H), 1.56 |
| | | | | (d, J = 3.4 Hz, 2H). |
| 202 | ** | ** | 624 | 1H NMR (400 MHz, DMSO-d6) δ 8.43 (s, 1H), 8.12 |
| | | | | (s, 1H), 7.29 (ddd, J = 11.7, 8.9, 4.3 Hz, 2H), 7.05- |
| | | | | 6.93 (m, 2H), 5.52 (d, J = 10.0 Hz, 1H), 4.77 (s, 1H), |
| | | | | 4.61 (s, 1H), 4.55-4.47 (m, 1H), 3.87 (ddd, J = 31.2, |
| | | | | 16.4, 8.9 Hz, 3H), 3.70-3.62 (m, 1H), 3.58 (d, J = |
| | | | | 4.2 Hz, 2H), 3.45 (s, 2H), 3.32(s, 2H), 3.01-2.91 (m, |
| | | | | 3H), 2.78 (s, 1H), 2.39 (dt, J = 23.8, 10.1 Hz, 4H), |
| | | | | 2.14 (d, J = 11.1 Hz, 1H), 2.08 (dd, J = 14.0, 9.3 Hz, |
| | | | | 2H), 2.00-1.89 (m, 2H), 1.81 (d, J = 11.4 Hz, 2H), |
| | | | | 1.68 (dd, J = 18.0, 9.3 Hz, 1H), 1.56 (d, J = 3.5 Hz, 2H). |
| 203 | **** | ** | 467 | 1H NMR (400 MHz, DMSO-d6) δ13.09 (s, 1H), 8.34 |
| | | | | (s, 1H), 8.12 (s, 1H), 7.33-7.24 (m, 2H), 7.04-6.90 |
| | | | | (m, 2H), 6.19-6.11 (d, J = 29.2 Hz, 1H), 4.77-4.69 (d, , |
| | | | | J = 31 Hz 1H), 4.52-4.49 (m, 1H), 3.92-3.88 (m, |
| | | | | 2H), 2.98-2.81 (m, 4H), 2.49 (d, J = 2.4 Hz, 2H), 2.08 |
| | | | | (d, J = 20 Hz, 2H), 1.81-1.78 (m, 2H). |
| 204 | *** | * | 553 | 1H NMR (400 MHz, DMSO) δ 8.43 (s, 1H), 8.12 (s, |
| | | | | 1H), 7.35-7.25 (m, 2H), 7.00 (dd, J = 11.8, 5.5 Hz, |
| | | | | 1H), 5.52 (d, J = 8.2 Hz, 1H), 4.71 (s, 1H), 4.65 (d, |
| | | | | J = 6.1 Hz, 1H), 4.59 (s, 1H), 4.52 (d, J = 3.7 Hz, 1H), |
| | | | | 4.22 (d, J = 8.4 Hz, 2H), 3.94 (d, J = 11.6 Hz, 1H), |
| | | | | 3.84 (s, 1H), 3.77-3.60 (m, 2H), 3.32 (s, 2H), 3.04- |
| | | | | 2.87 (m, 3H), 2.81 (s, 1H), 2.20-2.00 (m, 3H), 1.96 |
| | | | | (d, J = 9.8 Hz, 2H), 1.81 (d, J = 11.4 Hz, 2H), 1.68 (d, |
| | | | | J = 6.0 Hz, 1H), 1.56 (s, 2H). |
| 205 | *** | ** | 511 | 1H NMR (400 MHz, DMSO-d6) δ 7.82 (t, J = 7.8 Hz, |
| | | | | 1H), 7.49 (dd, J = 8.6, 6.5 Hz, 1H), 7.40 (dd, J = 8.9, |
| | | | | 2.6 Hz, 1H), 7.36 (d, J = 7.3 Hz, 1H), 7.19 (td, J = |
| | | | | 8.5, 2.6 Hz, 1H), 6.82 (d, J = 8.3 Hz, 1H), 4.61 (d, J = |
| | | | | 19.9 Hz, 2H), 3.87 (s, 3H), 3.79 (dd, J = 11.0, 5.6 Hz, |
| | | | | 2H), 3.53 (s, 2H), 3.12 (s, 4H), 2.91 (t, J = 5.8 Hz, |
| | | | | 1H), 2.78 (t, J = 5.8 Hz, 1H), 2.36 (d, J = 16.5 Hz, |
| | | | | 4H), 2.12 (d, J = 11.5 Hz, 3H). |
| 206 | *** | ** | 512 | 1H NMR (400 MHz, DMSO-d6) δ 8.22 (s, 1H), 7.33- |
| | | | | 7.24(d, J = 11.2, Hz, 2H), 7.03-6.98 (m, 1H), 4.51- |
| | | | | 4.50 (m, 1H), 4.40 (s, 3H), 4.15(t, J = 20.8 Hz, 2H), |
| | | | | 3.74-3.70 (m, 2H), 3.59 (t, J = 7.2 Hz, 2H), 3.19 (t, J = |
| | | | | 20 Hz, 4H), 2.97-2.91 (m, 2H), 2.82 (t, J = 15.2 Hz |
| | | | | 2H), 2.08-2.02 (m, 2H), 1.84-1.79 (t, 2H), 1.60- |
| | | | | 1.57(m, 4H). |
| 207 | ** | ** | 530 | 1H NMR (400 MHz, DMSO-d6) δ 13.11 (s, 1H), 8.34 |
| | | | | (s, 1H), 8.11 (s, 1H), 7.29 (ddd, J = 11.7, 9.2, 4.2 Hz, |
| | | | | 2H), 7.05-6.97 (m, 1H), 4.98-4.89 (m, 1H), 4.80 (t, |
| | | | | J = 7.4 Hz, 2H), 4.74 (t, J = 6.7 Hz, 2H), 4.51 (d, J = |
| | | | | 3.9 Hz, 1H), 4.40 (s, 2H), 3.62 (t, J = 5.8 Hz, 2H), |
| | | | | 3.32 (s, 2H), 2.96 (t, J = 9.6 Hz, 2H), 2.89 (t, J = 5.5 |
| | | | | Hz, 2H), 2.04 (s, 2H), 1.80 (d, J = 8.7 Hz, 2H). |
| 208 | *** | ** | 508 | 1H NMR (400 MHz, DMSO-d6) δ 8.59-8.52 (m, |
| | | | | 1H), 8.25-8.19 (m, 1H), 7.36-7.23 (m, 2H), 7.07- |
| | | | | 6.95 (m, 1H), 6.09-5.99 (m, 1H), 4.69-4.58 (m, |
| | | | | 2H), 4.56-4.47 (m, 1H), 3.84-3.76 (m, 2H), 3.42- |
| | | | | 3.31 (m, 2H, overlapping), 3.02-2.88 (m, 3H), 2.78 |
| | | | | (t, J = 5.7 Hz, 1H), 2.13 (s, 1.5H, overlapping), 2.12 |
| | | | | (s, 1.5H, overlapping), 2.10-2.02 (m, 2H, |
| | | | | overlapping), 1.92-1.85 (m, 2H, overlapping), 1.84 |
| | | | | (s, 1.5H, overlapping), 1.83 (s, 1.5H, overlapping). |
| 209 | *** | ** | 508 | 1H NMR (400 MHz, DMSO-d6) δ 8.44-8.36 (m, |
| | | | | 1H), 8.09-7.99 (m, 1H), 7.36-7.21 (m, 2H), 7.06- |
| | | | | 6.87 (m, 1H), 4.67-4.59 (m, 2H), 4.55-4.47 (m, |
| | | | | 1H), 4.08-4.03 (m, 2H), 3.83-3.74 (m, 2H), 3.32- |
| | | | | 3.28 (m, 2H, overlapping), 3.01-2.87 (m, 3H), 2.77 |
| | | | | (t, J = 5.8 Hz, 1H), 2.13 (s, 1.5H, overlapping), 2.12 |
| | | | | (s, 1.5H, overlapping), 2.11-2.03 (m, 2H, |
| | | | | overlapping), 1.90-1.79 (m, 2H), 1.34-1.22 (m, |
| | | | | 1H), 0.59-0.51 (m, 2H), 0.44-0.37 (m, 2H). |
| 210 | ** | ** | 546 | 1H NMR (400 MHz, DMSO-d6) δ 8.00 (td, J = 7.8, |
| | | | | 1.1 Hz, 1H), 7.75 (d, J = 7.3 Hz, 1H), 7.57 (d, J = 7.8 |
| | | | | Hz, 1H), 7.33-7.18 (m, 2H), 7.02-6.92 (m, 1H), |
| | | | | 6.42 (br. s, 1H), 6.25 (td, J = 55.2, 3.1 Hz, 1H), 4.93- |
| | | | | 4.79 (m, 1H), 4.65 (s, 1H), 4.60 (s, 1H), 4.49-4.39 |
| | | | | (m, 1H), 3.80 (q, J = 5.6 Hz, 2H), 3.57-3.30 |
| | | | | (overlapping m, 2H), 3.05-2.89 (m, 3H), 2.80 (t, J = |
| | | | | 5.8 Hz, 1H), 2.14 (s, 1.7H), 2.10 (s, 1.3H), 1.87- |
| | | | | 1.77 (m, 2H), 1.62-1.45 (m, 2H). |
| 211 | **** | ** | 505 | 1H NMR (400 MHz, DMSO-d6) δ 8.44-8.36 (m, |
| | | | | 1H), 8.09-7.99 (m, 1H), 7.36-7.21 (m, 2H), 7.06- |
| | | | | 6.87 (m, 1H), 4.67-4.59 (m, 2H), 4.55-4.47 (m, |
| | | | | 1H), 4.08-4.03 (m, 2H), 3.83-3.74 (m, 2H), 3.32- |
| | | | | 3.28 (m, 2H, overlapping), 3.01-2.87 (m, 3H), 2.77 |
| | | | | (t, J = 5.8 Hz, 1H), 2.13 (s, 1.5H, overlapping), 2.12 |
| | | | | (s, 1.5H, overlapping), 2.11-2.03 (m, 2H, |
| | | | | overlapping), 1.90-1.79 (m, 2H), 1.34-1.22 (m, |
| | | | | 1H), 0.59-0.51 (m, 2H), 0.44-0.37 (m, 2H). |
| 212 | ** | ** | 524 | 1H NMR (400 MHz, DMSO-d6) δ 8.50-8.46 (m, |
| | | | | 1H), 8.16-8.11 (m, 1H), 7.34-7.26 (m, 2H), 7.05- |
| | | | | 6.96 (m, 1H), 5.14-5.04 (m, 1H), 4.68-4.59 (m, |
| | | | | 2H), 4.57-4.46 (m, 1H), 3.82-3.76 (m, 2H), 3.72 (t, |
| | | | | J = 7.3 Hz, 2H), 3.46-3.39 (m, 2H), 3.31-3.25 (m, |
| | | | | 2H, overlapping), 3.01-2.88 (m, 3H), 2.77 (t, J = 5.7 |
| | | | | Hz, 1H), 2.35 (s, 3H), 2.13 (s, 1.5H, overlapping), |
| | | | | 2.11 (s, 1.5H, overlapping), 2.10-2.02 (m, 2H, |
| | | | | overlapping), 1.88-1.77 (m, 2H). Rotamers present. |
| 213 | ** | ** | 428 | 1H NMR (400 MHz, DMSO-d6) δ 9.10 (s, 1H), 8.55 |
| | | | | (d, J = 5.7 Hz, 1H), 8.46 (s, 1H), 8.13 (s, 1H), 7.61 (d, |
| | | | | J = 5.7 Hz, 1H), 3.95 (s, 3H), 3.43-3.31 (overlapped |
| | | | | m, 4H), 2.57-2.51 (overlapped m, 4H), 2.20 (d, J = |
| | | | | 7.3 Hz, 2H), 2.05-1.92 (m, 2H), 1.86-1.76 (m, |
| | | | | 3H), 1.76-1.60 (m, 2H), 1.11 (m, 2H). |
| 214 | ** | ** | 428 | 1H NMR (400 MHz, DMSO-D6) δ 9.10 (s, 1H), 8.55 |
| | | | | (d, J = 5.7 Hz, 1H), 8.45 (s, 1H), 8.13 (s, 1H), 7.61 (d, |
| | | | | J = 5.7 Hz, 1H), 3.95 (s, 3H), 3.44-3.35 (overlapped |
| | | | | m, 4H), 2.56-2.51 (overlapped m, 4H), 2.30-2.18 |
| | | | | (m, 2H), 2.17-2.06 (m, 1H), 2.06-1.92 (m, 1H), |
| | | | | 1.86-1.63 (m, 4H), 1.51-1.32 (m, 2H), 1.01-0.87 |
| | | | | (m, 1H). |
| 215 | *** | ** | 527 | 1H NMR (400 MHz, DMSO-d6) δ 8.14 (d, J = 1.7 |
| | | | | Hz, 1H), 7.33-7.19 (m, 2H), 7.05-6.92 (m, 1H), |
| | | | | 4.62 (m, J = 20.0 Hz, 2H), 4.54-4.39 (m, 1H), 3.96 |
| | | | | (s, 3H), 3.95 (s, 3H), 3.85-3.73 (m, 2H), 3.46-3.42 |
| | | | | (m, 2H), 3.09-2.97 (m, 2H), 2.92 (t, J = 5.6 Hz, 1H), |
| | | | | 2.79 (t, J = 5.9 Hz, 1H), 2.13 (s, 1.63H), 2.11 (s, |
| | | | | 1.35H), 1.96-1.82 (m, 2H), 1.67-1.49 (m, 2H). |
| 216 | *** | ** | 447 | 1H NMR (400 MHz, DMSO-d6) δ 9.12 (s, 1H), 8.49 |
| | | | | (s, 1H), 8.28 (s, 1H), 8.14 (s, 1H), 7.51-7.42 (m, |
| | | | | 1H), 7.21 (td, J = 10.0, 2.5 Hz, 1H), 7.07 (td, J = 8.5, |
| | | | | 2.2 Hz, 1H), 3.95 (s, 3H), 3.56 (s, 2H), 3.50-3.41 |
| | | | | (m, 4H), 2.58-2.52 (m, 4H). |
| 217 | ** | ** | 507 | 1H-NMR (400 MHz, DMSO-d6) δ 7.37-7.24 (m, |
| | | | | 2H), 7.02 (ddd, J = 11.0, 3.6, 2.2 Hz, 1H), 4.59 |
| | | | | (overlapping d, J = 18.0 Hz, 2H), 4.56-4.48 |
| | | | | (overalapping m, 1H), 3.76 (t, J = 5.5 Hz, 2H), 3.32- |
| | | | | 3.23 (m, 2H), 3.15-3.04 (m, 1H), 3.02-2.92 (m, |
| | | | | 2H), 2.87 (t, J = 5.7 Hz, 1H), 2.74 (t, J = 5.7 Hz, 1H), |
| | | | | 2.16-2.01 (overlapping m, 8H), 2.01-1.92 (m, 1H), |
| | | | | 1.91-1.73 (m, 6H). |
| 218 | ** | ** | 521 | 1H-NMR (400 MHz, DMSO-d6) δ 7.38-7.24 (m, |
| | | | | 2H), 7.07-6.97 (m, 1H), 4.61 (overlapping d, J = |
| | | | | 19.2 Hz, 2H), 4.57-4.48 (overlapping m, 1H), 3.77 |
| | | | | (t, J = 5.4 Hz, 2H), 3.43 (overlapping t, J = 13.5 Hz, |
| | | | | 3H), 3.30-3.23 (overlapping m, 2H), 3.09 (d, J = |
| | | | | 12.3 Hz, 2H), 3.02-2.91 (m, 2H), 2.88 (t, J = 5.6 |
| | | | | Hz, 1H), 2.75 (t, J = 5.5 Hz, 1H), 2.31-2.15 (m, 2H), |
| | | | | 2.14-1.96 (m, 7H), 1.95-1.81 (m, 2H). |
| 219 | *** | * | 471 | 1H NMR (400 MHz, DMSO-d6) δ 7.39 (d, J = 3.8 |
| | | | | Hz, 1H), 7.33-7.25 (m, 2H), 7.05-6.97 (m, 1H), |
| | | | | 4.55 (overlapping d, J = 17.5 Hz, 2H), 4.52-4.46 |
| | | | | (overlapping m, 1H), 4.07-4.00 (m, 2H), 3.75 (q, J = |
| | | | | 5.7 Hz, 2H), 3.58-3.46 (m, 2H), 3.07-2.97 (m, |
| | | | | 2H), 2.84 (t, J = 5.7 Hz, 1H), 2.73-2.65 (m, 1H), |
| | | | | 2.42 (br s, 2H), 2.11 (s, 1.5 H), 2.09 2.0 (s, 1.5H), 6- |
| | | | | 1.98 (m, 2H), 1.96-1.86 (m, 2H), 1.77-1.66 (m, 2H). |
| 220 | **** | ** | 483 | 1H NMR (400 MHz, DMSO-d6) δ 8.36 (s, 1H), 8.10- |
| | | | | 7.98 (m, 1H), 7.36-7.20 (m, 2H), 7.07-6.91 (m, |
| | | | | 1H), 4.68-4.57 (m, 2H), 4.56-4.45 (m, 1H), 4.29- |
| | | | | 4.16 (m, 2H), 3.83-3.72 (m, 2H), 3.31-3.26 (m, |
| | | | | 2H, overlapping), 3.02-2.86 (m, 3H), 2.76 (t, J = 5.6 |
| | | | | Hz, 1H), 2.12 (s, 1.5H, overlapping), 2.11 (s, 1.5H, |
| | | | | overlapping), 2.10-2.01 (m, 2H, overlapping), 1.90- |
| | | | | 1.76 (m, 2H), 1.40 (t, J = 7.3 Hz, 3H). |
| 221 | ** | ** | 509 | 1H NMR (400 MHz, DMSO-d6) δ 9.22 (d, J = 0.5 |
| | | | | Hz, 1H), 8.81 (s, 1H), 8.48 (s, 1H), 8.20 (d, J = 0.5 |
| | | | | Hz, 1H), 7.32-7.24 (m, 2H), 7.03-6.97 (m, 1H), |
| | | | | 4.59 (s, 1H), 3.98 (s, 3H), 3.92 (d, J = 10.6 Hz, 3H), |
| | | | | 3.76-3.67 (m, 2H), 3.36 (dd, J = 11.3, 7.0 Hz, 2H), |
| | | | | 1.96 (d, J = 25.2 Hz, 2H), 1.71-1.59 (m, 2H). |
| 222 | ** | ** | 508 | 1H NMR (400 MHz, DMSO-d6) δ9.18 (d, J = 0.7 Hz, |
| | | | | 1H), 8.94 (d, J = 4.8 Hz, 1H), 8.80 (s, 1H), 8.48 (s, |
| | | | | 1H), 8.14 (d, J = 0.6 Hz, 1H), 7.33-7.24 (m, 2H), |
| | | | | 7.01 (dd, J = 14.2, 5.0 Hz, 1H), 4.59 (s, 1H), 3.99 (s, |
| | | | | 3H), 3.73 (d, J = 15.4 Hz, 2H), 3.33 (s, 2H), 2.88 (d, |
| | | | | J = 4.9 Hz, 3H), 1.93 (s, 2H), 1.65 (d, J = 8.5 Hz, 3H). |
| 223 | *** | ** | 550 | 1HNMR (400 MHz, DMSO-d6) δ 8.19-8.11 (m, |
| | | | | 1H), 7.90-7.83 (m, 1H), 7.32-7.28 (m, 1H), 7.27- |
| | | | | 7.22 (m, 2H), 7.02-6.94 (m, 1H), 4.63 (d, J = 19.8 |
| | | | | Hz, 2H), 4.50-4.46 (m, 1H), 3.88-3.74 (m, 2H), |
| | | | | 3.45-3.39 (m, 2H), 3.10-2.97 (m, 2H), 2.93 (m1H), |
| | | | | 2.80 (t, J = 5.8 Hz, 1H), 2.13 (s, 1.70H), 2.11 (s, |
| | | | | 1.41H), 1.92-1.79 (m, 2H), 1.65-1.49 (m, 2H). |
| | | | | Rotamers present. |
| 224 | ** | ** | 436 | 1H NMR (400 MHz, DMSO-d6) δ 9.11 (s, 1H), 8.55 |
| | | | | (d, J = 5.7 Hz, 1H), 8.45 (s, 1H), 8.13 (s, 1H), 7.62 (d, |
| | | | | J = 5.7 Hz, 1H), 7.55-7.44 (m, 1H), 7.21 (tt, J = |
| | | | | 16.5, 8.2 Hz, 1H), 7.08 (tt, J = 7.3, 3.6 Hz, 1H), 4.25- |
| | | | | 4.12 (m, 1H), 3.95 (s, 3H), 3.57-3.53 (m, 2H), 3.41- |
| | | | | 3.34 (overlapping m, 2H), 2.79-2.69 (m, 1H), 2.53 |
| | | | | (d, J = 3.3 Hz, 2H), 2.41-2.29 (m, 1H), 1.12 (s, |
| | | | | 1.5H), 1.10 (s, 1.5H) |
| 225 | *** | ** | 436 | 1H NMR (400 MHz, DMSO-d6) δ 9.11 (s, 1H), 8.55 |
| | | | | (d, J = 5.7 Hz, 1H), 8.45 (s, 1H), 8.13 (s, 1H), 7.62 (d, |
| | | | | J = 5.7 Hz, 1H), 7.53-7.43 (m, 1H), 7.21 (td, J = |
| | | | | 10.0, 2.5 Hz, 1H), 7.08 (td, J = 8.5, 2.3 Hz, 1H), 4.25- |
| | | | | 4.12 (m, 1H), 3.95 (s, 3H), 3.63-3.48 (m, 2H), |
| | | | | 3.41-3.34 (overlapping m, 2H), 2.78-2.67 (m, 1H), |
| | | | | 2.55-2.51 (m, 2H), 2.43-2.28 (m, 1H), 1.11 (s, |
| | | | | 1.5H), 1.10 (s, 1.5H). |
| 226 | **** | ** | 437 | 1H NMR (400 MHz, DMSO-d6) δ 8.60 (s, 1H), 8.28 |
| | | | | (s, 1.79H)*, 8.00 (s, 1H), 7.52-7.42 (m, 1H), 7.21 |
| | | | | (td, J = 10.0, 2.5 Hz, 1H), 7.07 (td, J = 8.4, 2.4 Hz, |
| | | | | 1H), 6.45 (s, 1H), 6.23 (s, 2H), 3.91 (s, 3H), 3.57 (s, |
| | | | | 2H), 3.30-3.18 (m, 4H), 2.59-2.52 (overlapped m, |
| | | | | 4H). *79% of formate salt formation observed |
| 227 | *** | ** | 456 | 1H NMR (400 MHz, DMSO-d6) δ 8.91 (s, 1H), 8.54 |
| | | | | (s, 1H), 8.21 (s, 1H), 7.84 (s, 1H), 7.52-7.42 (m, |
| | | | | 1H), 7.26-7.17 (m, 1H), 7.12-7.03 (m, 1H), 3.96 |
| | | | | (s, 3H), 3.59 (s, 2H), 3.32-3.26 (submerged m, 4H), |
| | | | | 2.63-2.56 (m, 4H). |
| 228 | *** | ** | 437 | 1H-NMR (400 MHz, DMSO-d6) δ 9.01 (s, 1H), 8.48 |
| | | | | (s, 1H), 8.14 (s, 1H), 7.49 (s, 1H), 7.36-7.25 (m, |
| | | | | 2H), 7.07-6.98 (m, 1H), 4.62-4.52 (m, 1H), 3.95 |
| | | | | (s, 3H), 3.67-3.57 (m, 2H), 3.27-3.18 (m, 2H), |
| | | | | 2.60 (s, 3H), 2.13-2.00 (m, 2H), 1.89-1.77 (m, 2H). |
| 229 | ** | ** | 496 | 1H NMR (400 MHz, DMSO-d6) δ 13.06 (s, 1H), 8.32 |
| | | | | (s, 1H), 8.09 (s, 1H), 7.65 (s, 1H), 7.34-7.23 (m, |
| | | | | 2H), 7.04-6.97 (m, 1H), 4.50 (s, 1H), 3.59 (s, 2H), |
| | | | | 3.52-3.46 (m, 1H), 3.36 (dd, J = 13.8, 5.6 Hz, 1H), |
| | | | | 3.29-3.25 (m, 2H), 3.18 (dd, J = 9.3, 6.3 Hz, 1H), |
| | | | | 2.92 (dd, J = 17.9, 8.5 Hz, 2H), 2.82 (d, J = 5.3 Hz, |
| | | | | 2H), 2.76 (t, J = 8.2 Hz, 2H), 2.43-2.32 (m, 1H), |
| | | | | 2.22 (dd, J = 16.4, 7.8 Hz, 1H), 2.04 (s, 2H), 1.85- |
| | | | | 1.74 (m, 2H). |
| 230 | ** | ** | 465 | 1H-NMR (400 MHz, DMSO-d6) δ 9.06 (s, 1H), 8.75 |
| | | | | (s, 1H), 8.44 (s, 1H), 7.47 (s, 1H), 7.33-7.22 (m, |
| | | | | 2H), 7.03-6.95 (m, 1H), 4.60-4.50 (m, 1H), 3.97 |
| | | | | (s, 3H), 3.70-3.60 (m, 2H), 3.31-3.22 (overlapping |
| | | | | m, 2H), 2.61 (s, 3H), 1.96-1.84 (m, 2H), 1.67-1.55 |
| | | | | (m, 2H). |
| 231 | ** | ** | 482 | 1H NMR (400 MHz, DMSO-d6) δ 8.34 (s, 1H), 8.11 |
| | | | | (d, J = 3.6 Hz, 1H), 7.50 (dd, J = 15.5, 8.5 Hz, 1H), |
| | | | | 7.22 (td, J = 10.0, 2.5 Hz, 1H), 7.08 (td, J = 8.5, 2.4 |
| | | | | Hz, 1H), 4.66 (app. d, J = 5.0 Hz, 1H), 4.62 (s, 1H), |
| | | | | 3.91 (s, 1.5H), 3.90 (s, 1.5H), 3.68-3.69 (m, 2H), |
| | | | | 3.68-3.48 (m, 3H), 3.46-3.36 (submerged m, 2H), |
| | | | | 3.11-2.99 (m, 1H), 2.99-2.83 (m, 1H), 2.85-2.67 |
| | | | | (m, 2H), 2.65-2.55 (m, 1H), 2.27 (t, J = 9.1 Hz, 1H), |
| | | | | 2.13 (s, 1.5H), 2.11 (s, 1.5H), 0.88 (d, J = 3.6 Hz, |
| | | | | 1.5H), 0.87 (d, J = 3.6 Hz, 1.5H). |
| 232 | ** | ** | 482 | 1H NMR NMR (400 MHz, DMSO-d6) δ 8.34 (s, |
| | | | | 1H), 8.11 (d, J = 3.5 Hz, 1H), 7.50 (dd, J = 15.5, 8.4 |
| | | | | Hz, 1H), 7.22 (td, J = 10.1, 2.4 Hz, 1H), 7.08 (td, J = |
| | | | | 8.4, 2.2 Hz, 1H), 4.66 (d, J = 4.9 Hz, 1H), 4.62 (s, |
| | | | | 1H), 3.96-3.89 (m, 3H), 3.88-3.68 (m, 2H), 3.63- |
| | | | | 3.49 (m, 3H), 3.17-2.98 (m, 1H), 2.99-2.82 (m, |
| | | | | 1H), 2.85-2.65 (m, 3H), 2.65-2.55 (m, 1H), 2.60- |
| | | | | 2.46 (submerged m, 1H), 2.27 (t, J = 9.2 Hz, 1H), |
| | | | | 2.13 (s, 1.5H), 2.11 (s, 1.5H), 0.88 (d, J = 3.5 Hz, |
| | | | | 1.5H), 0.87 (d, J = 3.7 Hz, 1.5H). |
| 233 | ** | ** | 511 | 1H-NMR (400 MHz, DMSO-d6) δ 8.59 (s, 1H), 8.28 |
| | | | | (d, J = 2.0 Hz, 1H), 7.31-7.21 (m, 2H), 7.03-6.93 |
| | | | | (m, 1H), 4.63 (d, J = 19.7 Hz, 2H), 4.53-4.45 (m, |
| | | | | 1H), 4.38 (q, J = 7.0 Hz, 2H), 3.86-3.74 (m, 2H), |
| | | | | 3.47-3.39 (overlapping m, 2H), 3.10-2.99 (m, 2H), |
| | | | | 2.94 (t, J = 5.7 H, 1 Hz), 2.81 (t, J = 5.7 Hz, 1H), 2.14 |
| | | | | (overlapping s, 1.5H), 2.11 (overlapping s, 1.5H), |
| | | | | 1.92-1.81 (m, 2H), 1.62-1.50 (m, 2H), 1.37 (t, J = |
| | | | | 7.0 Hz, 3H). |
| 234 | ** | ** | 485 | 1H-NMR (400 MHz, DMSO-d6) δ 7.37-7.22 (m, |
| | | | | 2H), 7.08-6.95 (m, 1H), 6.24 (s, 1H), 4.89-4.78 |
| | | | | (m, 1H), 4.55 (overlapping d, J = 18.7 Hz, 2H), 4.52- |
| | | | | 4.45 (m, 1H), 4.19 (bs, 1H), 3.76 (q, J = 5.5 Hz, 2H), |
| | | | | 3.61-3.61 (m, 2H), 3.12-2.98 (m, 2H), 2.85 (t, J = |
| | | | | 5.7 Hz, 1H), 2.72 (t, J = 5.6 Hz, 1H), 2.41-2.28 (m, |
| | | | | 2H), 2.11 (overlapping s, 1.5H), 2.09 (overlapping s, |
| | | | | 1.5H), 2.07-1.93 (m, 2H), 1.88-1.78 (m, 2H), 1.76- |
| | | | | 1.44 (m, 4H). |
| 235 | ** | ** | 497 | 1H NMR (400 MHz, DMSO-d6) δ 9.37 (s, 1H), 7.58 |
| | | | | (s, 1H), 7.35-7.22 (m, 2H), 7.08-6.93 (m, 1H), |
| | | | | 4.70 (s, 1H), 4.65 (s, 1H), 4.56-4.43 (m, 1H), 4.09 |
| | | | | (s, 3H), 3.87-3.75 (m, 2H), 3.44-3.36 (overlapping |
| | | | | m, 2H), 3.10-3.00 (m, 2H), 2.97 (t, J = 5.8 Hz, 1H), |
| | | | | 2.83 (t, J = 5.8 Hz, 1H), 2.14 (s, 1.7H), 2.11 (s, 1.3H), |
| | | | | 1.99-1.86 (m, 2H), 1.74-1.57 (m, 2H). |
| 236 | ** | ** | 473 | 1H NMR (400 MHz, DMSO-d6) δ 7.37-7.23 (m, |
| | | | | 2H), 7.12-6.94 (m, 1H), 4.59 (d, J = 14.7 Hz, 2H), |
| | | | | 4.55-4.50 (m, 1H), 3.94-3.80 (m, 2H), 3.76 (t, J = |
| | | | | 5.7 Hz, 2H), 3.49-3.35 (overlapping m, 2H), 3.30- |
| | | | | 3.23 (overlapping m, 2H), 3.19-3.09 (m, 1H), 3.04- |
| | | | | 2.93 (m, 2H), 2.87 (t, J = 6.5 Hz, 1H), 2.78-2.71 (m, |
| | | | | 1H), 2.11 (s, 1.5H), 2.09 (s, 1.5H), 2.08-2.02 |
| | | | | (overlapping m, 2H), 1.91-1.76 (m, 4H), 1.71-1.63 |
| | | | | (m, 2H). |
| 237 | ** | ** | 473 | 1H NMR (400 MHz, DMSO-d6) δ 7.36-7.25 (m, |
| | | | | 2H), 7.05-6.97 (m, 1H), 4.60 (d, J = 15.7 Hz, 2 H), |
| | | | | 4.57-4.49 (m, 1H), 3.99-3.90 (m, 2H), 3.80-3.74 |
| | | | | (m, 2H), 3.46 (t, J = 11.9 Hz, 2H), 3.31-3.23 |
| | | | | (overlapping m, 2H), 3.22-3.11 (m, 1H), 3.02-2.93 |
| | | | | (m, 2H), 2.87 (t, J = 5.6 Hz, 1H), 2.74 (t, J = 5.7 Hz, |
| | | | | 1H), 2.11 (overlapping d, J = 1.2 Hz, 3H), 2.09-2.02 |
| | | | | (overlapping m, 2H), 1.88-1.74 (m, 4H), 1.68-1.58 |
| | | | | (m, 2H). |
| 238 | ** | ** | 466 | 1H NMR (400 MHz, DMSO-d6) δ 9.07 (s, 1H), 8.51 |
| | | | | (d, J = 5.7 Hz, 1H), 8.41 (s, 1H), 8.09 (s, 1H), 7.57 (d, |
| | | | | J = 5.7 Hz, 1H), 7.46 (dd, J = 15.4, 8.5 Hz, 1H), 7.22 |
| | | | | (td, J = 10.0, 2.5 Hz, 1H), 7.08 (td, J = 8.5, 2.4 Hz, |
| | | | | 1H), 4.29 (br. s, 1H), 3.94 (s, 3H), 3.74-3.66 (m, |
| | | | | 1H), 3.59-3.47 (m, 3H), 3.42-3.34 (m, 2H), 2.89 |
| | | | | (s, 3H), 2.85 (d, J = 10.5 Hz, 1H), 2.72 (d, J = 11.2 |
| | | | | Hz, 1H), 2.43 (dd, J = 11.4, 3.6 Hz, 1H), 2.29 (td, J = |
| | | | | 11.2, 2.7 Hz, 1H). |
| 239 | ** | ** | 532 | 1H NMR (400 MHz, DMSO-d6) δ 8.96 (d, J = 3.4 |
| | | | | Hz, 1H), 8.57 (d, J = 2.9 Hz, 1H), 7.65 (t, j = 71.9 Hz, |
| | | | | 1H), 7.33-7.19 (m, 2H), 7.05-6.93 (m, 1H), 4.65 |
| | | | | (d, J = 18.8 Hz, 2H), 4.55-4.43 (m, 1H), 3.82-3.80 |
| | | | | (m, 2H), 3.46-3.42 (m, 2H), 3.10-3.07 (m, 2H), 2.95 |
| | | | | (t, J = 5.7 Hz, 1H), 2.82 (t, J = 5.8 Hz, 1H), 2.14 (s, |
| | | | | 1.76H), 2.11 (s, 1.47H), 1.93-1.78 (m, 2H), 1.65- |
| | | | | 1.51 (m, 2H). Rotamers present. |
| 240 | ** | ** | 466 | 1H NMR (400 MHz, DMSO-d6) δ 9.07 (s, 1H), 8.51 |
| | | | | (d, J = 5.7 Hz, 1H), 8.41 (s, 1H), 8.09 (s, 1H), 7.57 (d, |
| | | | | J = 5.7 Hz, 1H), 7.46 (dd, J = 15.5, 8.4 Hz, 1H), 7.22 |
| | | | | (td, J = 10.2, 2.5 Hz, 1H), 7.08 (td, J = 8.4, 2.1 Hz, |
| | | | | 1H), 4.29 (br. s, 1H), 3.94 (s, 3H), 3.70 (t, J = 9.2 Hz, |
| | | | | 1H), 3.61-3.47 (m, 2H), 3.44-3.33 (overlapping m, |
| | | | | 2H), 2.89 (s, 3H), 2.85 (d, J = 10.0 Hz, 1H), 2.72 (d, |
| | | | | J = 11.1 Hz, 1H), 2.43 (dd, J = 11.3, 3.5 Hz, 1H), 2.29 |
| | | | | (td, J = 11.3, 2.3 Hz, 1H) |
| 241 | * | * | 394 | 1H NMR (400 MHz, DMSO-d6) δ 9.10 (s, 1H), 8.55 |
| | | | | (d, J = 5.7 Hz, 1H), 8.45 (s, 1H), 8.13 (s, 1H), 7.61 (d, |
| | | | | J = 5.7 Hz, 1H), 3.95 (s, 3H), 3.82 (d, J = 10.9 Hz, |
| | | | | 1H), 3.73 (d, J = 11.1 Hz, 1H), 3.32 (overlapped s, |
| | | | | 4H), 3.30-3.24 (m, 1H), 3.04 (dd, J = 10.9, 9.3 Hz, |
| | | | | 1H), 2.58-2.51 (overlapped m, 2H), 2.48-2.41 |
| | | | | (overlapped m, 2H), 2.25-2.09 (m, 2H), 1.83-1.71 |
| | | | | (m, 2H), 1.59-1.40 (m, 2H), 1.22-1.10 (m, 1H). |
| 242 | * | ** | 394 | 1H NMR (400 MHz, DMSO-d6) δ 9.10 (s, 1H), 8.55 |
| | | | | (d, J = 5.7 Hz, 1H), 8.45 (s, 1H), 8.13 (s, 1H), 7.61 (d, |
| | | | | J = 5.7 Hz, 1H), 3.95 (s, 3H), 3.82 (dd, J = 11.2, 2.8 |
| | | | | Hz, 2H), 3.35-3.29 (over lapped m, 8H), 3.29-3.22 |
| | | | | (overlapped m, 2H), 2.20 (d, J = 7.3 Hz, 2H), 1.83- |
| | | | | 1.72 (m, 1H), 1.61 (d, J = 12.9 Hz, 2H), 1.19-1.05 |
| | | | | (m, 2H). |
| 243 | ** | ** | 501 | 1H NMR (400 MHz, DMSO-d6) δ 9.16 (s, 1H), 8.79 |
| | | | | (d, J = 3.5 Hz, 1H), 7.35-7.18 (m, 2H), 7.07-6.85 |
| | | | | (m, 1H), 4.66 (d, J = 21.0 Hz, 2H), 4.57-4.45 (m, |
| | | | | 1H), 3.88-3.76 (m, 2H), 3.52-3.41 (m, 2H), 3.13- |
| | | | | 3.01 (m, 2H), 2.96 (t, J = 5.8 Hz, 1H), 2.83 (t, J = 5.9 |
| | | | | Hz, 1H), 2.14 (s, 1.5H), 2.11 (s, 1.5H), 2.00-1.84 |
| | | | | (m, 2H), 1.73-1.57 (m, 2H). |
| 244 | ** | ** | 512 | 1H NMR (400 MHz, DMSO-d6) δ 8.25 (s, 1H), 8.04- |
| | | | | 7.98 (m, 1H), 7.54-7.43 (m, 1H), 7.22 (td, J = |
| | | | | 10.2, 2.5 Hz, 1H), 7.08 (td, J = 8.5, 2.2 Hz, 1H), 4.66- |
| | | | | 4.62 (m, 1H), 4.60 (d, J = 7.7 Hz, 1H), 3.90 |
| | | | | (overlapping s, 1.2H), 3.89 (overlapping s, 1.8H), |
| | | | | 3.88-3.64 (m, 2H), 3.63-3.45 (m, 2H), 3.43-3.36 |
| | | | | (overlapping m, 2H), 3.42-3.26 (overlapping m, |
| | | | | 2H), 3.22-3.07 (m, 1H), 3.02 (overlapping s, 1.2H), |
| | | | | 3.01 (overlapping s, 1.8H), 2.99-2.89 (m, 1H), 2.89- |
| | | | | 2.75 (m, 1H), 2.75-2.52 (m, 3H), 2.45-2.36 (m, |
| | | | | 1H), 2.13 (overlapping s, 1.7H), 2.11 (overlapping s, 1.3H). |
| 245 | *** | ** | 512 | 1H NMR (400 MHz, DMSO-d6) δ 8.18 (s, 1H), 7.40- |
| | | | | 7.19 (m, 2H), 7.11-6.87 (m, 1H), 5.05 (d, J = 7.5 |
| | | | | Hz, 2H), 4.61 (d, J = 18.5 Hz, 2H), 4.54-4.47 (m, |
| | | | | 1H), 4.27-4.15 (m, 2H), 4.14-4.03 (m, 2H), 3.82- |
| | | | | 3.68 (m, 2H), 3.38-3.33 (overlapping m, 2H), 2.98 |
| | | | | (t, J = 9.3 Hz, 2H), 2.88 (t, J = 5.4 Hz, 1H), 2.76 (t, |
| | | | | J = 5.9 Hz, 1H), 2.12 (s, 1.5H), 2.11 (s, 1.5H), 2.08- |
| | | | | 1.98 (m, 2H), 1.87-1.72 (m, 2H). |
| 246 | * | * | 394 | 1H NMR (400 MHz, DMSO-d6) δ 9.10 (d, J = 0.5 |
| | | | | Hz, 1H), 8.55 (d, J = 5.7 Hz, 1H), 8.45 (s, 1H), 8.13 |
| | | | | (s, 1H), 7.66-7.58 (m, 1H), 3.95 (s, 3H), 3.86-3.79 |
| | | | | (m, 1H), 3.42 (m, 1H), 3.39-3.34 (m, 4H), 3.33- |
| | | | | 3.26 (m, 1H), 2.65-2.52 (m, 4H), 2.41 (dd, J = 13.0, |
| | | | | 6.6 Hz, 1H), 2.30 (dd, J = 12.9, 4.5 Hz, 1H), 1.82- |
| | | | | 1.68 (m, 1H), 1.59 (d, J = 13.2 Hz, 1H), 1.49-1.37 |
| | | | | (m, 3H), 1.22-1.07 (m, 1H). |
| 247 | *** | ** | 499 | 1H-NMR (400 MHz, DMSO-d6) δ 7.35-7.25 (m, |
| | | | | 2H), 7.03-6.98 (m, 1H), 6.31 (s), 4.56 (overlapping |
| | | | | d, J = 19.2 Hz, 2H), 4.49-4.46 (overalapping m, |
| | | | | 1H), 3.92-3.85 (m, 1H), 3.76 (q, J = 5.6 Hz, 2H), |
| | | | | 3.59-3.48 (m, 2H), 3.30 (s, 3H), 3.13-2.99 (m, |
| | | | | 2H), 2.85 (t, J = 5.7 Hz, 1H), 2.72 (t, J = 5.7 Hz, 1H), |
| | | | | 2.45-2.34 (m, 2H), 2.11 (s, 1.5H), 2.09 (s, 1.5H), |
| | | | | 2.05-1.92 (m, 2H), 1.90-1.51 (m, 6H). |
| 248 | ** | ** | 501 | 1H-NMR (400 MHz, DMSO-d6) δ 7.36-7.24 (m, |
| | | | | 2H), 7.06-6.97 (m, 1H), 4.59 (overlapping d, J = |
| | | | | 16.1 Hz, 2H), 4.56-4.48 (overlapping m, 1H), 3.81- |
| | | | | 3.70 (m, 2H), 3.31-3.24 (overlapping m, 3H), 3.24 |
| | | | | (overlapping s, 3H), 3.03-2.90 (m, 3H), 2.87 (t, J = |
| | | | | 5.8 Hz, 1H), 2.74 (t, J = 5.7 Hz, 1H), 2.11 |
| | | | | (overlapping s, 1.5H), 2.10 (overlapping s, 1.5H), |
| | | | | 2.10-1.97 (overlapping m, 4H), 1.88-1.74 (m, 3H), |
| | | | | 1.72-1.60 (m, 1H), 1.49-1.31 (m, 3H), 1.18-1.01 |
| | | | | (m, 1H). |
| 249 | ** | ** | 512 | 1H NMR (400 MHz, DMSO-d6) δ 8.25 (s, 1H), 8.06- |
| | | | | 7.95 (m, 1H), 7.53-7.41 (m, 1H), 7.22 (td, J = |
| | | | | 10.0, 2.5 Hz, 1H), 7.08 (td, J = 8.5, 2.3 Hz, 1H), 4.71- |
| | | | | 4.49 (m, 2H), 3.91-3.88 (m, 3H), 3.88-3.65 (m, |
| | | | | 2H), 3.61-3.50 (m, 2H), 3.44-3.35 (overlapping m, |
| | | | | 2H), 3.34-3.26 (overlapping m, 2H), 3.23-3.06 (m, |
| | | | | 1H), 3.02 (overlapping s, 1.3H), 3.01 (overlapping s, |
| | | | | 1.7H), 2.99-2.90 (m, 1H), 2.89-2.75 (m, 1H), 2.74- |
| | | | | 2.52 (m, 3H), 2.47-2.36 (m, 1H), 2.13 |
| | | | | (overlapping s, 1.6H), 2.11 (overlapping s, 1.4H). |
| 250 | ** | ** | 456 | 1H NMR (400 MHz, DMSO-d6) δ 9.06 (s, 1H), 8.50 |
| | | | | (s, 1H), 8.16 (s, 1H), 7.64 (s, 1H), 7.39-7.23 (m, |
| | | | | 2H), 7.06-6.97 (m, 1H), 5.39 (d, J = 5.0 Hz, 1H), |
| | | | | 4.63-4.53 (m, 1H), 4.34-4.28 (m, 1H), 3.96 (s, |
| | | | | 3H), 3.70-3.60 (m, 2H), 3.30-3.18 (m, 2H), 2.14- |
| | | | | 2.03 (m, 2H), 1.90-1.77 (m, 2H), 1.28-1.20 (m, |
| | | | | 1H), 0.51-0.35 (m, 4H). |
| 251 | ** | ** | 481 | 1H NMR (400 MHz, DMSO) δ 9.16 (d, J = 0.4 Hz, |
| | | | | 1H), 8.56 (s, 1H), 8.23 (t, J = 4.5 Hz, 1H), 8.20 (s, |
| | | | | 2H), 7.36-7.27 (m, 2H), 7.06-6.99 (m, 1H), 4.64- |
| | | | | 4.55 (m, 1H), 3.97 (s, 3H), 3.93 (s, 3H), 3.78-3.66 |
| | | | | (m, 2H), 3.36 (s, 1H), 3.33 (s, 1H), 2.14-2.04 (m, |
| | | | | 2H), 1.91-1.79 (m, 2H). |
| 252 | *** | ** | 450 | 1H NMR (400 MHz, DMSO-d6) δ 8.27 (s, 1H), 8.03 |
| | | | | (s, 1H), 7.44 (td, J = 7.8, 1.8 Hz, 1H), 7.36-7.29 (m, |
| | | | | 1H), 7.22-7.14 (m, 2H), 4.61 (d, J = 15.5 Hz, 2H), |
| | | | | 3.91 (d, J = 1.7 Hz, 3H), 3.78 (dd, J = 10.1, 5.6 Hz, |
| | | | | 2H), 3.61 (s, 2H), 3.05 (s, 4H), 2.88 (t, J = 5.8 Hz, |
| | | | | 1H), 2.75 (t, J = 5.7 Hz, 1H), 2.69-2.65 (m, 1H), |
| | | | | 2.63-2.54 (m, 4H), 2.11 (d, J = 5.9 Hz, 3H). |
| 253 | ** | ** | 498 | 1H NMR (400 MHz, DMSO-d6) δ 8.37 (d, J = 2.1 |
| | | | | Hz, 1H), 8.08 (d, J = 2.8 Hz, 1H), 7.57 (dd, J = 9.0, |
| | | | | 2.5 Hz, 1H), 7.49 (dd, J = 8.5, 6.1 Hz, 1H), 7.33 (td, |
| | | | | J = 8.5, 2.5 Hz, 1H), 4.65 (s, 1H), 4.61 (s, 1H), 3.92 (d, |
| | | | | J = 1.7 Hz, 3H), 3.85 (s, 2H), 3.79 (d, J = 5.3 Hz, 2H), |
| | | | | 3.33 (s, 2H), 3.08 (d, J = 21.8 Hz, 4H), 2.90 (t, J = 5.6 |
| | | | | Hz, 1H), 2.77 (t, J = 5.6 Hz, 1H), 2.12 (d, J = 5.1 Hz, 3H). |
| 254 | ** | ** | 494 | 1H NMR (400 MHz, DMSO-d6) δ 8.38 (d, J = 2.4 |
| | | | | Hz, 1H), 8.08 (d, J = 2.9 Hz, 1H), 7.35 (t, J = 8.4 Hz, |
| | | | | 1H), 6.93 (dd, J = 11.9, 2.3 Hz, 1H), 6.87 (dd, J = 8.5, |
| | | | | 2.4 Hz, 1H), 4.63 (d, J = 16.6 Hz, 2H), 3.92 (d, J = |
| | | | | 1.8 Hz, 3H), 3.88-3.76 (m, 7H), 3.45 (d, J = 17.8 |
| | | | | Hz, 2H), 3.06 (d, J = 11.7 Hz, 4H), 2.90 (t, J = 5.6 Hz, |
| | | | | 1H), 2.77 (t, J = 5.7 Hz, 1H), 2.12 (d, J = 4.9 Hz, 3H). |
| 255 | ** | ** | 464 | 1H NMR (400 MHz, DMSO-d6) δ 8.36 (s, 1H), 8.08 |
| | | | | (d, J = 2.6 Hz, 1H), 7.54-7.47 (m, 2H), 7.32-7.26 |
| | | | | (m, 2H), 4.64 (d, J = 16.5 Hz, 2H), 3.92 (d, J = 1.7 |
| | | | | Hz, 3H), 3.84-3.74 (m, 4H), 3.57 (s, 2H), 3.09 (s, |
| | | | | 4H), 2.90 (t, J = 5.8 Hz, 1H), 2.77 (t, J = 5.8 Hz, 1H), |
| | | | | 2.13 (d, J = 5.2 Hz, 3H). |
| 256 | ** | ** | 450 | 1H NMR (400 MHz, DMSO-d6) δ 8.27 (s, |
| | | | | 1H), 8.16(s, 1H) 8.04 (d, J = 1.5 Hz, 1H), 7.37 (dd, J = |
| | | | | 14.1, 8.0 Hz, 1H), 7.17 (t, J = 9.8 Hz, 2H), 7.08 (t, J = |
| | | | | 8.3 Hz, 1H), 4.61 (d, J = 15.6 Hz, 2H), 3.90 (t, J = 5.9 |
| | | | | Hz, 3H), 3.82-3.75 (m, 2H), 3.58 (s, 2H), 3.07 (s, |
| | | | | 4H), 2.90 (d, J = 5.7 Hz, 1H), 2.76 (t, J = 5.8 Hz, 1H), |
| | | | | 2.69-2.66 (m, 1H), 2.65-2.53 (m, 4H), 2.12 (d, J = |
| | | | | 5.9 Hz, 3H). |
| 257 | ** | ** | 450 | 1H NMR (400 MHz, DMSO-d6) δ 8.27 (s, 1H), 8.13 |
| | | | | (s, 1H), 8.04 (s, 1H), 7.36 (d, J = 6.4 Hz, 3H), 7.15 (t, |
| | | | | J = 8.7 Hz, 2H), 4.61 (d, J = 15.5 Hz, 2H), 3.91 (d, J = |
| | | | | 1.6 Hz, 3H), 3.82-3.76 (m, 2H), 3.56 (s, 2H), 3.06 |
| | | | | (s, 4H), 2.89 (t, J = 5.7 Hz, 1H), 2.75 (dd, J = 12.4, |
| | | | | 6.4 Hz, 2H), 2.11 (d, J = 6.0 Hz, 3H). |
| 258 | * | * | 486 | 1H NMR (400 MHz, DMSO-d6) δ 7.58 (s, 1H), 7.37- |
| | | | | 7.24 (m, 2H), 7.10-6.95 (m, 1H), 4.90-4.80 (m, |
| | | | | J = 5.4 Hz, 1H), 4.67-4.57 (m, 2H), 4.57-4.44 (m, |
| | | | | 1H), 3.78 (t, J = 6.0 Hz, 2H), 3.28-3.18 (m, 1H), |
| | | | | 3.12-3.00 (m, 1H), 2.99-2.85 (m, 1H), 2.83-2.72 |
| | | | | (m, 1H), 2.22 (t, J = 5.5 Hz, 2H), 2.12 (s, 1.5H), 2.10 |
| | | | | (s, 1.5H), 1.99 (m, 3H overlapping), 1.78 (m, 5H |
| | | | | overlapping). Rotamers present. |
| 259 | * | * | 436 | 1H NMR (400 MHz, DMSO-d6) δ 9.42 (s, 1H), 8.78 |
| | | | | (d, J = 5.7 Hz, 1H), 8.36 (s, 1H), 8.07 (s, 1H), 7.93 (d, |
| | | | | J = 5.6 Hz, 1H), 7.59 (dd, J = 15.4, 8.5 Hz, 1H), 7.29 |
| | | | | (td, J = 10.2, 2.5 Hz, 1H), 7.14 (td, J = 8.4, 2.1 Hz, |
| | | | | 1H), 4.12-4.03 (m, 1H), 3.94 (s, 3H), 3.85-3.77 |
| | | | | (m, 1H), 3.77-3.70 (m, 2H), 3.30 (overlapped m, |
| | | | | 1H), 3.21 (d, J = 16.5 Hz, 1H), 3.09-3.00 (m, 1H), |
| | | | | 3.00-2.90 (m, 1H). |
| 260 | ** | ** | 541 | 1H NMR (400 MHz, DMSO-d6) δ 8.36 (s, 1H), 8.05 |
| | | | | (s, 1H), 7.34-7.21 (m, 2H), 7.07-6.93 (m, 1H), |
| | | | | 4.56-4.45 (m, 3H), 4.22 (q, J = 7.2 Hz, 2H), 3.75- |
| | | | | 3.63 (m, 2H), 3.33-3.27 (m, 2H, overlapping), 3.00- |
| | | | | 2.90 (m, 2H), 2.84-2.74 (m, 2H), 2.14-1.98 (m, |
| | | | | 2H), 1.88-1.75 (m, 2H), 1.43 (s, 9H, overlapping), |
| | | | | 1.40 (t, J = 7.3 Hz, 3H, overlapping). |
| 261 | *** | ** | 510 | 1H NMR (400 MHz, DMSO-d6) δ 9.02 (s, 1H), 8.45 |
| | | | | (s, 1H), 8.12 (s, 1H), 7.69 (s, 1H), 7.52-7.44 (m, |
| | | | | 1H), 7.22 (td, J = 10.0, 2.5 Hz, 1H), 7.08 (td, J = 8.4, |
| | | | | 2.2 Hz, 1H), 5.49 (bs, 1H), 4.76 (bs, 1H), 4.51 (s, |
| | | | | 1H), 3.94 (s, 3H), 3.58 (s, 2H), 3.53-3.08 |
| | | | | (overlapped m, 4H), 2.62-2.54 (overlapped m, 4H), |
| | | | | 1.07 (s, 3H), 1.04 (s, 3H). |
| 262 | *** | ** | 510 | 1H NMR (400 MHz, DMSO-d6) δ 9.02 (s, 1H), 8.45 |
| | | | | (s, 1H), 8.12 (d, J = 0.4 Hz, 1H), 7.69 (s, 1H), 7.52- |
| | | | | 7.43 (m, 1H), 7.26-7.18 (m, 1H), 7.08 (td, J = 8.5, |
| | | | | 2.2 Hz, 1H), 5.50 (bs, 1H), 4.76 (bs, 1H), 4.51 (s, |
| | | | | 1H), 3.94 (s, 3H), 3.58 (overlapped s, 2H), 3.52- |
| | | | | 3.15 (overlapped m, 4H), 2.62-2.54 (overlapped m, |
| | | | | 4H), 1.07 (s, 3H), 1.04 (s, 3H). |
| 263 | *** | ** | 511 | 1H NMR (400 MHz, DMSO-d6) δ 9.04 (s, 1H), 8.50 |
| | | | | (s, 1H), 8.16 (s, 1H), 7.70 (s, 1H), 7.36-7.26 (m, |
| | | | | 2H), 7.06-6.98 (m, 1H), 5.49 (d, J = 5.4 Hz, 1H), |
| | | | | 4.77 (s, 1H), 4.62-4.53 (m, 1H), 4.51 (d, J = 5.1 Hz, |
| | | | | 1H), 3.96 (s, 3H), 3.70-3.59 (m, 2H), 3.29-3.20 |
| | | | | (overlapped m, 2H), 2.14-2.03 (m, 2H), 1.91-1.78 |
| | | | | (m, 2H), 1.08 (s, 3H), 1.05 (s, 3H). |
| 264 | **** | ** | 546 | 1H NMR (400 MHz, DMSO-d6) δ 9.09 (s, 1H), 8.90 |
| | | | | (s, 1H), 8.43 (s, 1H), 7.94 (t, J = 58.7 Hz, 1H), 7.72 |
| | | | | (s, 1H), 7.51-7.43 (m, 1H), 7.21 (td, J = 10.2, 2.5 |
| | | | | Hz, 1H), 7.07 (td, J = 8.5, 2.2 Hz, 1H), 5.51 (d, J = |
| | | | | 5.3 Hz, 1H), 4.74 (s, 1H), 4.52 (d, J = 4.9 Hz, 1H), |
| | | | | 3.57 (s, 2H), 3.40-3.35 (overlapped m, 4H), 2.60- |
| | | | | 2.52 (overlapped m, 4H), 1.07 (s, 3H), 1.06 (s, 3H). |
| 265 | ** | ** | 472 | 1H NMR (400 MHz, DMSO-d6) δ 7.38-7.24 (m, |
| | | | | 2H), 7.07-6.94 (m, 1H), 4.63-4.56 (m, 2H), 4.56- |
| | | | | 4.47 (m, 1H), 3.81-3.71 (m, 2H), 3.30-3.21 (m, |
| | | | | 2H), 3.06-2.91 (overlapping, 5H), 2.87 (t, J = 5.8 |
| | | | | Hz, 1H), 2.73 (t, J = 5.5 Hz, 1H), 2.62-2.53 (m, 2H), |
| | | | | 2.11 (s, 1.5H), 2.10 (s, 1.5H), 2.09-2.02 (m, 2H), |
| | | | | 1.91-1.72 (m, 2H), 1.75-1.52 (overlapping, m, 4H). |
| 266 | **** | ** | 547 | 1H NMR (400 MHz, DMSO-d6) δ 8.36 (s, 1H), 8.05 |
| | | | | (s, 1H), 7.34-7.25 (m, 2H), 7.05-6.97 (m, 1H), |
| | | | | 4.55-4.48 (m, 1H), 4.45 (s, 2H), 4.23 (q, J = 7.3 Hz, |
| | | | | 2H), 3.67 (t, J = 5.8 Hz, 2H), 3.54-3.45 (m, 1H), |
| | | | | 3.38-3.34 (overlapping m, 1H), 3.32-3.28 |
| | | | | (overlapping m, 1H), 3.01-2.92 (m, 2H), 2.88 (t, J = |
| | | | | 5.8 Hz, 2H), 2.11-1.97 (m, 2 Hz), 1.90-1.76 (m, |
| | | | | 2H), 1.40 (t, J = 7.3 Hz, 3H), 1.25 (d, J = 6.8 Hz, 6H). |
| 267 | *** | ** | 533 | 1H NMR (400 MHz, DMSO-d6) δ 8.36 (s, 1H), 8.05 |
| | | | | (s, 1H), 7.36-7.23 (m, 2H), 7.13-6.86 (m, 1H), |
| | | | | 4.57-4.46 (m, 1H), 4.41 (s, 2H), 4.22 (q, J = 7.3 Hz, |
| | | | | 2H), 3.62 (t, J = 5.9 Hz, 2H), 3.36-3.34 (overlapping |
| | | | | m, 1H), 3.32-3.28 (overlapping m, 1H), 3.21 (q, J = |
| | | | | 7.3 Hz, 2H), 3.01-2.93 (m, 2H), 2.90 (t, J = 5.8 Hz, |
| | | | | 2H), 2.11-2.03 (m, 2H), 1.89-1.78 (m, 2H), 1.40 (t, |
| | | | | J = 7.3 Hz, 3H), 1.22 (t, J = 7.3 Hz, 3H). |
| 268 | **** | ** | 510 | 1H NMR (400 MHz, DMSO-d6) δ 9.01 (s, 1H), 8.56 |
| | | | | (s, 1H), 8.24 (s, 1H), 7.81 (s, 1H), 7.53-7.45 (m, |
| | | | | 1H), 7.26-7.19 (m, 1H), 7.12-7.04 (m, 1H), 5.48 |
| | | | | (s, 1H), 4.67 (s, 1H), 4.53 (s, 1H), 3.96 (s, 3H), 3.60 |
| | | | | (s, 2H), 3.29-3.21 (m, 4H), 2.65-2.57 (m, 4H), |
| | | | | 1.06 (s, 3H), 1.04 (s, 3H). |
| 269 | **** | ** | 519 | 1H NMR (400 MHz, DMSO-d6) δ 8.36 (s, 1H), 8.05 |
| | | | | (s, 1H), 7.40-7.21 (m, 2H), 7.13-6.90 (m, 1H), |
| | | | | 4.57-4.45 (m, 1H), 4.37 (s, 2H), 4.23 (q, J = 7.3 Hz, |
| | | | | 2H), 3.55 (t, J = 5.9 Hz, 2H), 3.38-3.34 (overlapping |
| | | | | m, 1H), 3.32-3.27 (overlapping m, 1H), 3.02 (s, 3H), |
| | | | | 3.01-2.90 (m, 4H), 2.13-1.99 (m, 2H), 1.89-1.77 |
| | | | | (m, 2H), 1.40 (t, J = 7.3 Hz, 3H). |
| 270 | *** | ** | 501 | 1H-NMR (400 MHz, DMSO-d6) δ 9.03 (s, 1H), 8.47 |
| | | | | (s, 1H), 8.30 (s, formate 1H), 8.12 (s, 1H), 7.69 (s, |
| | | | | 1H), 7.50-7.44 (m, 1H), 7.21 (td, J = 10.1, 2.5 Hz, |
| | | | | 1H), 7.07 (td, J = 8.5, 2.3 Hz, 1H), 4.51 (s, 1H), 4.24 |
| | | | | (q, J = 7.2 Hz, 2H), 3.58 (s, 2H), 3.41-3.35 |
| | | | | (overlapping m with H2O, 3H), 2.62-2.52 (m, 4H), |
| | | | | 1.42 (t, J = 7.3 Hz, 3H), 1.07 (overlapping s, 3H), |
| | | | | 1.05 (overlapping s, 3H). Diol H-peaks were |
| | | | | exchanged with deuterated solvent. |
| 271 | *** | ** | 508 | 1H NMR (400 MHz, DMSO-d6) δ 8.56-8.53 (m, |
| | | | | 1H), 8.21 (d, J = 3.5 Hz, 1H), 7.36-7.23 (m, 2H), |
| | | | | 7.06-6.97 (m, 1H), 6.09-5.99 (m, 1H), 4.69-4.60 |
| | | | | (m, 2H), 4.55-4.48 (m, 1H), 3.83-3.75 (m, 2H), |
| | | | | 3.32-3.27 (m, 2H, overlapping), 3.02-2.88 (m, |
| | | | | 3H), 2.78 (t, J = 5.8 Hz, 1H), 2.15-2.11 (m, 3H, |
| | | | | overlapping), 2.10-2.03 (m, 2H, overlapping), 1.91- |
| | | | | 1.87 (m, 2H, overlapping), 1.86-1.81 (m, 3H, |
| | | | | overlapping). |
| 272 | *** | ** | 508 | 1H NMR (400 MHz, DMSO-d6) δ 8.56-8.53 (m, |
| | | | | 1H), 8.21 (d, J = 3.7 Hz, 1H), 7.35-7.24 (m, 2H), |
| | | | | 7.06-6.97 (m, 1H), 6.09-5.97 (m, 1H), 4.68-4.59 |
| | | | | (m, 2H), 4.56-4.48 (m, 1H), 3.83-3.75 (m, 2H), |
| | | | | 3.32-3.26 (m, 2H, overlapping), 3.03-2.88 (m, |
| | | | | 3H), 2.78 (t, J = 5.4 Hz, 1H), 2.14-2.11 (m, 3H, |
| | | | | overlapping), 2.11-2.04 (m, 2H, overlapping), 1.91- |
| | | | | 1.85 (m, 2H, overlapping), 1.85-1.82 (m, 3H, |
| | | | | overlapping). |
| 273 | **** | ** | 499 | 1H NMR (400 MHz, DMSO-d6) δ 8.36 (s, 1H), 8.05 |
| | | | | (s, 1H), 7.34-7.25 (m, 2H), 7.05-6.97 (m, 1H), |
| | | | | 4.55 (s, 2H, overlapping), 4.53-4.47 (m, 1H, |
| | | | | overlapping), 4.22 (q, J = 7.3 Hz, 2H), 3.73 (t, J = 5.8 |
| | | | | Hz, 2H), 3.65 (s, 3H), 3.31-3.27 (m, 2H, |
| | | | | overlapping), 3.01-2.91 (m, 2H), 2.82 (t, J = 5.7 Hz, |
| | | | | 2H), 2.11-2.02 (m, 2H), 1.89-1.77 (m, 2H), 1.40 (t, |
| | | | | J = 7.3 Hz, 3H). |
| 274 | **** | ** | 466 | 1H NMR (400 MHz, DMSO-d6) δ 8.35 (s, 1H), 8.03 |
| | | | | (s, 1H), 7.36-7.23 (m, 2H), 7.09-6.94 (m, 1H), |
| | | | | 4.55-4.47 (m, 1H), 4.43 (s, 2H), 4.21 (q, J = 7.3 Hz, |
| | | | | 2H), 3.60 (t, J = 5.9 Hz, 2H), 3.38-3.34 (m, 1H), |
| | | | | 3.32-3.29 (m, 1H), 3.02-2.89 (m, 4H), 2.14-1.96 |
| | | | | (m, 2H), 1.92-1.73 (m, 2H), 1.40 (t, J = 7.3 Hz, 3H). |
| 275 | **** | *** | 513 | 1H-NMR (400 MHz, DMSO-d6) δ 8.37 (s, 1H), 8.06 |
| | | | | (s, 1H), 7.35-7.25 (m, 2H), 7.05-6.97 (m, 1H), |
| | | | | 4.61 (s, 2H), 4.55-4.47 (m, 1H), 4.27-4.18 (m, |
| | | | | 4H), 3.80 (t, J = 5.6 Hz, 1H), 3.74 (t, J = 5.4 Hz, 1H), |
| | | | | 3.33-3.27 (overlapping m, 5H), 3.01-2.92 (m, 2H), |
| | | | | 2.91-2.86 (m, 1H), 2.82-2.75 (m, 1H), 2.11-2.02 |
| | | | | (m, 2H), 1.88-1.78 (m, 2H), 1.40 (t, J = 7.3 Hz, 3H). |
| 276 | **** | *** | 527 | 1H-NMR (400 MHz, DMSO-d6) δ 8.37 (s, 1H), 8.06 |
| | | | | (s, 1H), 7.34-7.26 (m, 2H), 7.04-6.97 (m, 1H), |
| | | | | 5.55 (s, 1H), 5.16 (s, 1H), 4.60 (s, 1H), 4.55-4.46 |
| | | | | (m, 1H), 4.22 (q, J = 7.2 Hz, 2H), 4.30-4.20 |
| | | | | (submerged m, 1H) 3.83 (s, 1H), 3.33-3.24 |
| | | | | (overlapping m, 2H), 3.01-2.72 (overlapping m, |
| | | | | 4H), 2.11-2.00 (m, 2H), 1.88-1.76 (m, 2H), 1.41 (t, |
| | | | | J = 7.3 Hz, 3H), 1.34 (s, 6H). |
| 277 | *** | ** | 438 | 1H NMR (400 MHz, DMSO) δ 8.61 (s, 1H), 8.34 (s, |
| | | | | 1H), 8.03 (s, 1H), 7.34-7.26 (m, 2H), 7.05-6.98 |
| | | | | (m, 1H), 6.46 (s, 1H), 6.23 (s, 2H), 4.57-4.53 (m, 1H), |
| | | | | 3.93 (s, 3H), 3.59-3.51 (m, 2H), 3.16-3.11 (m, 2H), |
| | | | | 2.07-2.04 (m, 2H), 1.86-1.76 (m, 2H). |
| 278 | ** | ** | 447 | 1H NMR (400 MHz, DMSO-d6) δ 8.26 (s, 1H), 8.00 |
| | | | | (s, 1H), 7.41-7.33 (m, 4H), 7.30-7.24 (m, 1H), |
| | | | | 4.93 (dd, J = 7.9, 4.0 Hz, 1H), 3.88 (s, 3H), 3.71 (s, |
| | | | | 2H), 3.51 (s, 2H), 3.41-3.34 (m, 1H), 3.05 (d, J = |
| | | | | 12.3 Hz, 1H), 2.91-2.76 (m, 6H), 2.01 (s, 3H), 1.91 |
| | | | | (s, 1H), 1.79 (s, 1H), 1.67 (d, J = 9.5 Hz, 1H), 1.56 (d, |
| | | | | J = 8.9 Hz, 1H). |
| 279 | ** | ** | 487.5 | 1H NMR(400 MHz, DMSO-d6) δ 7.37-7.24 (m, |
| | | | | 2H), 7.07-6.96 (m, 1H), 4.66-4.56 (m, 2H), 4.56- |
| | | | | 4.46 (m, 1 Hz), 3.96-3.85 (m, 1H), 3.81-3.68 (m, |
| | | | | 2H), 3.44-3.34 (m, 1H), 3.31-3.23 (m, 2H), 3.21 |
| | | | | (s, 3H), 3.02-2.91 (m, 2H), 2.90-2.83 (m, 1H), 2.78- |
| | | | | 2.71 (m, 1H), 2.39-2.24 (m, 1H), 2.11 (s, 1.69), |
| | | | | 2.11 (s, 1.30H), 2.10-2.01 (m, 2H), 1.93-1.68 |
| | | | | (overlapped, m, 7H). |
| 280 | ** | ** | 432 | 1H NMR (400 MHz, DMSO-d6) δ 8.27 (s, 1H), 8.04 |
| | | | | (d, J = 1.5 Hz, 1H), 7.32 (t, J = 6.5 Hz, 4H), 7.28- |
| | | | | 7.23 (m, 1H), 4.61 (d, J = 15.5 Hz, 2H), 3.91 (d, J = |
| | | | | 1.8 Hz, 3H), 3.81-3.75 (m, 2H), 3.55 (s, 2H), 3.06 |
| | | | | (s, 4H), 2.89 (t, J = 5.8 Hz, 1H), 2.75 (t, J = 5.8 Hz, |
| | | | | 1H), 2.54 (d, J = 10.0 Hz, 4H), 2.11 (d, J = 5.9 Hz, 3H). |
| 281 | **** | ** | 500 | 1H NMR (400 MHz, DMSO-d6) δ 8.29 (s, 1H), 8.06 |
| | | | | (d, J = 1.9 Hz, 1H), 7.57 (d, J = 2.6 Hz, 1H), 7.49 (d, |
| | | | | J = 8.5 Hz, 1H), 7.37 (dd, J = 8.5, 2.7 Hz, 1H), 4.62 (d, |
| | | | | J = 15.9 Hz, 2H), 3.92 (d, J = 1.7 Hz, 3H), 3.82-3.76 |
| | | | | (m, 2H), 3.66 (s, 2H), 3.08 (s, 4H), 2.90 (t, J = 5.7 Hz, |
| | | | | 1H), 2.76 (t, J = 5.7 Hz, 1H), 2.66 (dd, J = 8.4, 6.6 |
| | | | | Hz, 4H), 2.12 (d, J = 5.6 Hz, 3H). |
| 282 | * | * | 494 | 1H NMR (400 MHz, DMSO-d6) δ 8.36 (s, 1H), 8.08 |
| | | | | (d, J = 2.8 Hz, 1H), 7.36-7.28 (m, 1H), 7.28-7.21 |
| | | | | (m, 2H), 4.64 (d, J = 16.6 Hz, 2H), 3.90 (d, J = 14.0 |
| | | | | Hz, 6H), 3.83-3.76 (m, 2H), 3.70 (s, 4H), 3.08 (s, |
| | | | | 4H), 2.90 (t, J = 5.5 Hz, 1H), 2.77 (t, J = 5.9 Hz, 1H), |
| | | | | 2.16-2.07 (m, 3H). |
| 283 | ** | ** | 476 | 1H NMR (400 MHz, DMSO-d6) δ 8.36 (s, 1H), 8.07 |
| | | | | (d, J = 2.9 Hz, 1H), 7.40 (d, J = 8.7 Hz, 2H), 7.02- |
| | | | | 6.97 (m, 2H), 4.63 (d, J = 16.6 Hz, 2H), 3.92 (d, J = |
| | | | | 1.7 Hz, 3H), 3.78 (d, J = 9.3 Hz, 5H), 3.69 (s, 4H), |
| | | | | 3.07 (s, 4H), 2.91 (d, J = 5.3 Hz, 1H), 2.77 (d, J = 5.1 |
| | | | | Hz, 1H), 2.12 (d, J = 5.1 Hz, 3H). |
| 284 | ** | ** | 477 | 1H NMR (400 MHz, DMSO-d6) δ 8.38 (d, J = 2.1 |
| | | | | Hz, 1H), 8.29 (d, J = 2.6 Hz, 1H), 8.08 (d, J = 3.0 Hz, |
| | | | | 1H), 7.63 (d, J = 8.6 Hz, 1H), 7.50 (dd, J = 8.7, 2.9 |
| | | | | Hz, 1H), 4.63 (d, J = 16.3 Hz, 2H), 3.92 (d, J = 1.7 |
| | | | | Hz, 3H), 3.88 (s, 3H), 3.86-3.76 (m, 4H), 3.74 (s, |
| | | | | 2H), 3.08 (d, J = 17.2 Hz, 4H), 2.90 (s, 1H), 2.77 (s, |
| | | | | 1H), 2.12 (d, J = 4.9 Hz, 3H). |
| 285 | * | ** | 482 | 1H NMR (400 MHz, DMSO-d6) δ 8.34 (s, 1H), 8.07 |
| | | | | (d, J = 2.6 Hz, 1H), 7.41-7.33 (m, 1H), 7.22-7.14 |
| | | | | (m, 2H), 4.63 (d, J = 16.6 Hz, 2H), 3.91 (s, 3H), 3.79 |
| | | | | (d, J = 3.7 Hz, 4H), 3.50 (s, 2H), 3.10 (s, 4H), 2.90 (d, |
| | | | | J = 5.4 Hz, 1H), 2.77 (s, 1H), 2.12 (d, J = 5.4 Hz, 3H). |
| 286 | ** | * | 489 | 1H NMR (400 MHz, DMSO-d6) δ 8.37 (d, J = 2.0 |
| | | | | Hz, 1H), 8.08 (d, J = 2.8 Hz, 1H), 8.00 (dd, J = 8.8, |
| | | | | 1.8 Hz, 1H), 7.73-7.65 (m, 2H), 4.63 (d, J = 16.5 |
| | | | | Hz, 2H), 3.91 (d, J = 1.8 Hz, 3H), 3.89-3.82 (m, |
| | | | | 2H), 3.82-3.76 (m, 2H), 3.44 (s, 2H), 3.09 (d, J = |
| | | | | 20.9 Hz, 4H), 2.90 (t, J = 5.5 Hz, 1H), 2.78 (d, J = 5.5 |
| | | | | Hz, 1H), 2.15-2.07 (m, 3H). |
| 287 | * | * | 506 | 1H NMR (400 MHz, DMSO-d6) δ 8.36 (s, 1H), 8.06 |
| | | | | (d, J = 2.9 Hz, 1H), 7.13 (d, J = 8.3 Hz, 1H), 6.63- |
| | | | | 6.55 (m, 2H), 4.62 (d, J = 16.4 Hz, 2H), 3.91 (s, 3H), |
| | | | | 3.79 (s, 10H), 3.06 (s, 2H), 2.99 (s, 2H), 2.90 (s, 1H), |
| | | | | 2.76 (s, 1H), 2.12 (d, J = 5.3 Hz, 3H). |
| 288 | **** | ** | 469 | 1H NMR (400 MHz, DMSO-d6) δ 9.11 (s, 1H), 8.55 |
| | | | | (overlapping d, J = 5.7 Hz, 1H), 8.55 (overlapping s, |
| | | | | 1H), 8.17 (s, 1H), 7.63 (d, J = 5.7 Hz, 1H), 7.46 (d, |
| | | | | J = 8.5 Hz, 1H), 7.41 (d, J = 2.3 Hz, 1H), 7.03 (dd, J = |
| | | | | 8.5, 2.3 Hz, 1H), 4.97-4.68 (m, 1H), 4.26 (q, J = 7.3 |
| | | | | Hz, 2H), 3.71-3.53 (m, 2H), 3.44-3.26 |
| | | | | (overlapping m, 2H), 2.20-1.98 (m, 2H), 1.97-1.72 |
| | | | | (m, 2H), 1.43 (t, J = 7.3 Hz, 3H). |
| 289 | ** | ** | 457 | 1H NMR (400 MHz, DMSO-d6) δ 8.92 (s, 1H), 8.49 |
| | | | | (s, 1H), 8.13 (s, 1H), 7.70 (s, 1H), 7.36-7.25 (m, |
| | | | | 2H), 7.06-6.97 (m, 1H), 4.63-4.53 (m, 1H), 3.95 (s, |
| | | | | 3H), 3.74-3.64 (m, 2H), 3.37-3.27 (overlapped m, |
| | | | | 2H), 2.12-2.00 (m, 2H), 1.87-1.76 (m, 2H). |
| 290 | *** | ** | 515 | 1H NMR (400 MHz, DMSO-d6) δ 8.33 (s, 1H, |
| | | | | overlapping), 8.33 (s, 1H, overlapping), 8.04 (s, 1H), |
| | | | | 7.34-7.25 (m, 2H), 7.05-6.96 (m, 1H), 4.57-4.45 |
| | | | | (m, 1H), 4.21 (q, J = 7.2 Hz, 2H), 3.73-3.67 (m, 1H, |
| | | | | overlapping), 3.67-3.59 (m, 2H, overlapping), 3.41- |
| | | | | 3.33 (m, 2H, overlapping), 3.33-3.25 (m, 2H, |
| | | | | overlapping), 2.93 (m, 2H), 2.87-2.83 (m, 1H, |
| | | | | overlapping), 2.82 (s, 3H, overlapping), 2.66-2.58 |
| | | | | (m, 1H), 2.53-2.51 (m, 2H, overlapping), 2.49- |
| | | | | 2.42 (m, 2H, overlapping), 2.16-2.00 (m, 2H), 1.91- |
| | | | | 1.75 (m, 2H), 1.40 (t, J = 7.2 Hz, 3H). |
| 291 | **** | ** | 538 | 1H NMR (400 MHz, DMSO-d6) δ 8.39-8.34 (m, |
| | | | | 1H), 8.07-8.03 (m, 1H), 7.34-7.25 (m, 2H), 7.05- |
| | | | | 6.96 (m, 1H), 4.82-4.55 (m, 2H), 4.55-4.45 (m, |
| | | | | 1H), 4.22 (q, J = 7.2 Hz, 2H), 3.98-3.73 (m, 3H), |
| | | | | 3.32-3.27 (m, 2H, overlapping), 3.01-2.90 (m, 3H, |
| | | | | overlapping), 2.90-2.74 (m, 2H, overlapping), 2.68- |
| | | | | 2.59 (m, 1H), 2.12-1.92 (m, 3H), 1.90-1.78 (m, |
| | | | | 2H), 1.73-1.52 (m, 3H), 1.41 (t, J = 7.2 Hz, 3H). |
| 292 | * | ** | 452 | 1H NMR (400 MHz, DMSO-d6) δ 400 MHz, DMSO- |
| | | | | d6) ? 9.05 (s, 1H), 8.50 (d, J = 5.7 Hz, 1H), 8.41 (s, |
| | | | | 1H), 8.10 (s, 1H), 7.54 (d, J = 5.7 Hz, 1H), 7.51- |
| | | | | 7.39 (m, 1H), 7.21 (td, J = 10.1, 2.5 Hz, 1H), 7.08 (td, |
| | | | | J = 8.5, 2.3 Hz, 1H), 4.50 (br. s, 1H), 4.22-4.12 (m, |
| | | | | 1H), 3.94 (s, 3H), 3.73 (t, J = 9.3 Hz, 1H), 3.60-3.55 |
| | | | | (overlapping m, 1H), 3.53 (overlapping s, , 2H), 3.48- |
| | | | | 3.37 (m, 1H), 3.37-3.29 (overlapping m, 1H), 2.83 |
| | | | | (d, J = 11.1 Hz, 1H), 2.75 (d, J = 11.2 Hz, 1H), 2.42 |
| | | | | (dd, J = 11.3, 3.6 Hz, 1H), 2.27 (td, J = 11.3, 2.8 Hz, 1H). |
| 293 | ** | ** | 448 | 1H NMR (400 MHz, DMSO-d6) δ 9.14 (s, 1H), 8.56 |
| | | | | (s, 1H), 8.31 (s, 1H), 8.18 (s, 1H), 7.36-7.27 (m, |
| | | | | 2H), 7.02 (dd, J = 12.0, 5.3 Hz, 1H), 4.61-4.58 (m, |
| | | | | 1H)), 3.96 (s, 3H), 3.78-3.69 (m, 2H), 3.37 (d, J = |
| | | | | 9.9 Hz, 2H), 2.11-2.02 (m, 2H), 1.87-1.77 (m, 2H). |
| 294 | **** | ** | 452 | 1H NMR (400 MHz, DMSO-d6) δ 8.61 (s, 1H), 8.37 |
| | | | | (s, 1H), 8.03 (s, 1H), 7.35-7.25 (m, 2H), 7.06-6.98 |
| | | | | (m, 1H), 6.46 (s, 1h), 6.23 (s, 2H), 4.59-4.51 (m, |
| | | | | 1H), 4.23 (q, J = 7.2 Hz, 2H), 3.58-3.49 (m, 2H), |
| | | | | 3.16-3.11 (m, 2H), 2.07-2.04 (m, 2H), 1.88-1.76 |
| | | | | (m, 2H), 1.41 (t, J = 7.2 Hz, 3H). |
| 295 | **** | ** | 453 | 1H NMR (400 MHz, DMSO-d6) δ 9.12 (s, 1H), 8.56 |
| | | | | (overlapping d, J = 5.1 Hz, 1H), 8.55 (overlapping s, |
| | | | | 1H), 8.17 (s, 1H), 7.63 (d, J = 5.6 Hz, 1H), 7.43 (dd, |
| | | | | J = 7.4, 2.5 Hz, 1H), 7.28 (dd, J = 11.1, 8.7 Hz, 1H), |
| | | | | 7.02 (ddd, J = 8.7, 3.8, 2.6 Hz, 1H), 4.84-4.64 (m, |
| | | | | 1H), 4.26 (q, J = 7.3 Hz, 2H), 3.72-3.56 (m, 2H), |
| | | | | 3.34-3.17 (overlapping m, 2H), 2.18-2.03 (m, 2H), |
| | | | | 1.91-1.78 (m, 2H), 1.43 (t, J = 7.3 Hz, 3H). |
| 296 | ** | ** | 480 | 1H NMR (400 MHz, DMSO-d6) δ 10.69 (s, 1H), |
| | | | | 8.90 (s, 1H), 8.45 (s, 1H), 8.29 (s, 1H), 8.12 (s, 1H), |
| | | | | 7.35-7.26 (m, 2H), 7.05-6.99 (m, 1H), 4.61-4.54 |
| | | | | (m, 1H), 3.95 (s, 3H), 3.69-3.61 (m, 2H), 3.29- |
| | | | | 3.21 (m, 2H), 2.15 (s, 3H), 2.15-2.06 (m, 2H), 1.88- |
| | | | | 1.79 (m, 2H). |
| 297 | ** | ** | 479 | 1H NMR (400 MHz, DMSO-d6) δ 10.68 (s, 1H), |
| | | | | 8.89 (d, J = 0.6 Hz, 1H), 8.38 (s, 1H), 8.27 (s, 1H), |
| | | | | 8.08 (d, J = 0.4 Hz, 1H), 7.53-7.43 (m, 1H), 7.21 |
| | | | | (td, J = 10.1, 2.5 Hz, 1H), 7.07 (td, J = 8.4, 2.0 Hz, |
| | | | | 1H), 3.94 (s, 3H), 3.57 (s, 2H), 3.41-3.33 |
| | | | | (overlapped m, 4H), 2.59-2.53 (m, 4H), 2.14 (s, 3H). |
| 298 | *** | ** | 538 | 1H NMR (400 MHz, DMSO-d6) δ 9.03 (d, J = 0.6 |
| | | | | Hz, 1H), 8.46 (s, 1H), 8.13 (s, 1H), 7.69 (s, 1H), 7.52- |
| | | | | 7.43 (m, 1H), 7.22 (td, J = 10.1, 2.5 Hz, 1H), 7.08 |
| | | | | (td, J = 8.5, 2.2 Hz, 1H), 5.48 (d, J = 4.7 Hz, 1H), |
| | | | | 4.76 (bs, 1H), 4.62 (hept, J = 6.6 Hz, 1H), 4.51 (d, J = |
| | | | | 3.4 Hz, 1H), 3.59 (s, 2H), 3.43-3.26 (overlapped m, |
| | | | | 4H), 2.60-2.53 (overlapped m, 4H), 1.47 (d, J = 6.7 |
| | | | | Hz, 6H), 1.08 (s, 3H), 1.05 (s, 3H). |
| 299 | **** | ** | 513 | 1H NMR (400 MHz, DMSO-d6) δ 8.36 (s, 1H), 8.05 |
| | | | | (s, 1H), 7.35-7.24 (m, 2H), 7.06-6.96 (m, 1H), |
| | | | | 4.60-4.46 (m, 3H), 4.22 (q, J = 7.3 Hz, 2H), 4.09 (q, |
| | | | | J = 7.0 Hz, 2H), 3.76-3.69 (m, 2H), 3.36-3.27 (m, |
| | | | | 2H, overlapping), 3.01-2.91 (m, 2H), 2.85-2.78 |
| | | | | (m, 2H), 2.11-2.02 (m, 2H), 1.89-1.78 (m, 2H), |
| | | | | 1.40 (t, J = 7.2 Hz, 3H), 1.22 (t, J = 7.1 Hz, 3H). |
| 300 | **** | ** | 512 | 1H NMR (400 MHz, DMSO-d6) δ 8.36 (s, 1H), 8.06- |
| | | | | 8.03 (m, 1H), 7.34-7.25 (m, 2H), 7.05-6.96 (m, |
| | | | | 1H), 4.55-4.47 (m, 1H), 4.31 (s, 2H), 4.22 (q, J = |
| | | | | 7.3 Hz, 2H), 3.52-3.46 (m, 2H), 3.32-3.27 (m, 2H, |
| | | | | overlapping), 3.00-2.91 (m, 2H), 2.89-2.83 (m, |
| | | | | 2H), 2.80 (s, 6H), 2.10-2.03 (m, 2H), 1.89-1.77 |
| | | | | (m, 2H), 1.40 (t, J = 7.3 Hz, 3H). |
| 301 | ** | ** | 407.2 | 1H NMR (400 MHz, CDCl3) δ 9.65 (d, J = 0.8 Hz, |
| | | | | 1H), 8.88 (d, J = 5.6 Hz, 1H), 7.96 (dd, J = 5.8, 0.9 |
| | | | | Hz, 1H), 7.91 (d, J = 0.7 Hz, 1H), 7.43 (d, J = 0.7 Hz, |
| | | | | 1H), 7.03 (td, J = 9.0, 5.3 Hz, 1H), 6.89 (ddd, J = |
| | | | | 11.2, 8.4, 3.0 Hz, 1H), 6.89 (ddd, J = 11.2, 8.4, 3.0 |
| | | | | Hz, 1H), 6.83-6.77 (m, 1H), 5.93 (d, J = 1.7 Hz, |
| | | | | 1H), 4.60 (s, 1H), 2.70 (d, J = 15.2 Hz, 2H), 2.60- |
| | | | | 2.47 (m, 2H), 2.16 (ddd, J = 18.7, 11.6, 5.2 Hz, 2H). |
| 302 | ** | ** | 455.2 | 1H NMR (400 MHz, CDCl3) δ 7.86 (d, J = 3.1 Hz, |
| | | | | 1H), 7.37 (t, J = 6.6 Hz, 1H), 7.01 (ddd, J = 15.7, |
| | | | | 11.0, 7.9 Hz, 1H), 6.86 (ddd, J = 11.2, 6.9, 3.0 Hz, |
| | | | | 1H), 6.81-6.75 (m, 1H), 4.96 (s, 1H), 4.83 (s, 1H), |
| | | | | 4.51 (s, 1H), 4.01 (dd, J = 8.0, 3.9 Hz, 2H), 3.87 (t, |
| | | | | J = 5.9 Hz, 1H), 3.12 (tt, J = 22.2, 6.0 Hz, 2H), 2.31- |
| | | | | 2.14 (m, 7H), 2.03-1.96 (m, 1H), 1.74 (t, J = 13.2 |
| | | | | Hz, 5H). |
| 303 | * | * | 453.2 | 1H NMR (400 MHz, CDCl3) δ 7.81 (s, 1H), 7.36- |
| | | | | 7.31 (m, 1H), 7.04-6.96 (m, 1H), 6.87 (ddd, J = |
| | | | | 11.1, 8.4, 2.9 Hz, 1H), 6.79 (t, J = 7.9 Hz, 1H), 5.77 |
| | | | | (d, J = 1.8 Hz, 1H), 4.99 (s, 1H), 4.85 (s, 1H), 4.56 (d, |
| | | | | J = 3.1 Hz, 1H), 4.02 (t, J = 6.0 Hz, 1H), 3.87 (t, J = |
| | | | | 5.9 Hz, 1H), 3.14 (dt, J = 19.1, 5.9 Hz, 2H), 2.59 (dd, |
| | | | | J = 31.7, 11.6 Hz, 2H), 2.51-2.39 (m, 2H), 2.23 (d, |
| | | | | J = 12.3 Hz, 3H), 2.19-2.01 (m, 3H). |
| 304 | ** | * | 407.2 | 1H NMR (400 MHz, CDCl3) δ 9.59 (d, J = 0.9 Hz, |
| | | | | 1H), 8.86 (d, J = 5.8 Hz, 1H), 8.04 (dd, J = 5.8, 0.9 |
| | | | | Hz, 1H), 7.93 (d, J = 0.7 Hz, 1H), 7.45 (d, J = 0.7 Hz, |
| | | | | 1H), 7.08-6.99 (m, 1H), 6.93-6.86 (m, 1H), 6.85- |
| | | | | 6.77 (m, 1H), 5.90 (s, 1H), 4.62 (s, 1H), 2.77-2.65 |
| | | | | (m, 2H), 2.64-2.47 (m, 2H), 2.15 (ddd, J = 21.0, |
| | | | | 11.5, 6.2 Hz, 2H). |
| 305 | *** | ** | 515.5 | 1H NMR(400 MHz, DMSO-d6) δ 8.34 (s, 1H), 8.05- |
| | | | | 7.99 (m, 1H), 7.34-7.24 (m, 2H), 7.07-6.95 (m, |
| | | | | 1H), 4.57-4.44 (m, 1H), 4.21 (q, J = 7.2 Hz, 2H), |
| | | | | 3.74-3.67 (m, 1H, overlapping), 3.67-3.58 (m, 2H, |
| | | | | overlapping), 3.40-3.33 (m, 2H, overlapping), 3.32- |
| | | | | 3.25 (m, 2H, overlapping), 2.99-2.88 (m, 2H), |
| | | | | 2.87-2.82 (m, 2H, overlapping), 2.82 (s, 2H, |
| | | | | overlapping), 2.65-2.58 (m, 1H), 2.53-2.51 (m, |
| | | | | 1H, overlapping), 2.48-2.42 (m, 2H, overlapping), |
| | | | | 2.11-2.01 (m, 2H), 1.89-1.78 (m, 2H), 1.40 (t, J = |
| | | | | 7.3 Hz, 3H). |
| 306 | *** | ** | 457.4 | 1H NMR(400 MHz, DMSO-d6) δ 7.36-7.24 (m, |
| | | | | 2H), 7.07-6.97 (m, 1H), 6.92-6.81 (m, 1H), 5.01- |
| | | | | 4.93 (m, 2H), 4.87-4.78 (m, 2H), 4.58 (d, J = 17.0 |
| | | | | Hz, 2H), 4.55-4.48 (overlapping m, 1H), 3.78 (t, J = |
| | | | | 5.3 Hz, 2H), 3.48-3.36 (m, 2H), 3.08-2.98 (m, |
| | | | | 2H), 2.95-2.88 (m, 1H), 2.77 (t, J = 5.7 Hz, 1H), |
| | | | | 2.12 (s, 1.5 H), 2.10 (s, 1.5H), 2.08-2.01 (m, 2H), |
| | | | | 1.87-1.71 (m, 2H). |
| 307 | *** | ** | 525.5 | 1H NMR(400 MHz, DMSO-d6) δ 9.04 (s, 1H), 8.54 |
| | | | | (s, 1H), 8.16 (s, 1H), 7.70 (s, 1H), 7.38-7.23 (m, |
| | | | | 2H), 7.06-6.96 (m, 1H), 5.48 (d, J = 5.4 Hz, 1H), |
| | | | | 4.77 (s, 1H), 4.62-4.54 (m, 1H), 4.51 (d, J = 5.4 Hz, |
| | | | | 1H), 4.26 (q, J = 7.2 Hz, 2H), 3.71-3.58 (m, 2H), |
| | | | | 3.30-3.20 (m, 2H), 2.15-2.02 (m, 2H), 1.90-1.77 |
| | | | | (m, 2H), 1.43 (t, J = 7.2 Hz, 3H), 1.08 (s, 3H), 1.05 (s, 3H). |
| 308 | **** | ** | 531.5 | 1H NMR(400 MHz, DMSO-d6) δ 8.46 (d, J = 3.6 Hz, |
| | | | | 1H), 7.29-7.18 (m, 2H), 7.00-6.90 (m, 1H), 4.66- |
| | | | | 4.55 (m, 2H), 4.53-4.41 (m, 1H), 3.99 (d, J = 1.3 |
| | | | | Hz, 3H), 3.82-3.72 (m, 2H), 3.49-3.37 (m, 2H), |
| | | | | 3.11-2.98 (m, 2H), 2.90 (t, J = 5.5 Hz, 1H), 2.77 (t, |
| | | | | J = 5.9 Hz, 1H), 2.10 (s, 1.5H), 2.07 (s, 1.5H), 1.92- |
| | | | | 1.77 (m, 2H), 1.63-1.43 (m, 2H). |
| 309 | *** | ** | 482.4 | 1H NMR(400 MHz, DMSO-d6) δ 9.06 (s, 1H), 8.50 |
| | | | | (s, 1H), 8.38 (formate bs, 1H), 8.19-8.13 (m, 1H), |
| | | | | 7.71 (s, 1H), 7.35-7.26 (m, 2H), 7.05-6.98 (m, |
| | | | | 1H), 4.88 (dd, J = 8.0, 3.1 Hz, 1H), 4.63-4.54 (m, |
| | | | | J = 7.8, 4.1 Hz, 1H), 3.96 (s, 3H), 3.72-3.57 (m, 2H), |
| | | | | 3.33-3.16 (m, 3H), 2.89 (dd, J = 12.7, 8.3 Hz, 1H), |
| | | | | 2.50 (residual DMSO, 0.5H) 2.14-2.02 (m, 2H), |
| | | | | 1.90-1.76 (m, 2H). |
| 310 | **** | ** | 495.4 | 1H NMR(400 MHz, DMSO-d6) δ 8.37 (s, 1H), 8.06 |
| | | | | (d, J = 7.7 Hz, 1H), 7.36-7.25 (m, 2H), 7.06-6.96 |
| | | | | (m, 1H), 6.96-6.87 (m, 1H), 6.21-6.12 (m, 1H), |
| | | | | 5.73 (t, J = 9.6 Hz, 1H), 4.77 (s, 1H), 4.69 (s, 1H), |
| | | | | 4.55-4.45 (m, 1H), 4.23 (q, J = 7.2 Hz, 2H), 3.96- |
| | | | | 3.82 (m, 2H), 3.31-3.26 (overlapping m, 2H), 2.96 |
| | | | | (t, J = 9.6 Hz, 2H), 2.90-2.84 (m, 1H), 2.84-2.77 |
| | | | | (m, 1H), 2.12-2.01 (m, 2H), 1.89-1.75 (m, 2H), |
| | | | | 1.41 (t, J = 7.2 Hz, 3H). |
| 311 | **** | ** | 499.4 | 1H NMR(400 MHz, DMSO-d6) δ 8.37 (s, 1H), 8.06 |
| | | | | (d, J = 3.7 Hz, 1H), 7.33-7.25 (m, 2H), 7.05-6.97 |
| | | | | (m, 1H), 4.72 (s, 1H), 4.63 (s, 1H), 4.57 (s, 1H), 4.54- |
| | | | | 4.46 (m, 1H), 4.26-4.16 (m, 4H), 3.83 (t, J = 5.4 |
| | | | | Hz, 1H), 3.71 (t, J = 5.5 Hz, 1H), 3.31-3.23 |
| | | | | (overlapping m, 2H), 3.00-2.91 (t, J = 9.2 Hz, 2H), |
| | | | | 2.89 (t, J = 6.0 Hz 1H), 2.79 (t, J = 4.0 Hz 1H), 2.11- |
| | | | | 2.01 (m, 2H), 1.88-1.78 (m, 2H), 1.41 (t, J = 7.3 Hz, 3H). |
| 312 | **** | ** | 497.5 | 1H NMR(400 MHz, DMSO-d6) δ 8.36 (s, 0.88H), |
| | | | | 8.05 (d, J = 1.9 Hz, 0.87H), 7.55 (t, J = 2.1 Hz, |
| | | | | 0.13H), 7.34-7.22 (m, 2H), 7.05-6.95 (m, 1H), |
| | | | | 6.65 (dd, J = 4.3, 1.9 Hz, 0.13H), 4.62 (d, J = 16.3 Hz, |
| | | | | 2H), 4.55-4.44 (m, 1H), 4.27-4.19 (m, 0.27H), |
| | | | | 4.16 (td, J = 6.8, 2.0 Hz, 1.71H), 3.85-3.73 (m, 2H), |
| | | | | 3.39-3.34 (m, 1H, overlapping with H2O), 3.32- |
| | | | | 3.26 (m, overlapping m with H2O, 1H), 3.02-2.86 |
| | | | | (m, 3H), 2.82-2.75 (m, 1H), 2.13 (overlapping s, |
| | | | | 1.5H), 2.11 (overlapping s, 1.5H), 2.10-2.02 (m, |
| | | | | 1.76H), 1.92-1.75 (m, 3.7H), 1.74-1.69 (m, 0.3H), |
| | | | | 1.66-1.56 (m, 0.3H), 0.83 (t, J = 7.4 Hz, 2.6H), 0.77 |
| | | | | (t, J = 7.4 Hz, 0.4H). |
| 313 | *** | ** | 451.4 | 1H NMR (400 MHz, DMSO-d6) δ 9.01 (s, 1H), 8.52 |
| | | | | (s, 1H), 8.14 (s, 1H), 7.52-7.46 (m, 1H), 7.35-7.24 |
| | | | | (m, 2H), 7.05-6.94 (m, 1H), 4.63-4.52 (m, 1H), |
| | | | | 4.26 (q, J = 7.2 Hz, 2H), 3.69-3.57 (m, 2H), 3.26- |
| | | | | 3.19 (m, 2H), 2.60 (s, 3H), 2.14-2.03 (m, 2H), 1.90- |
| | | | | 1.76 (m, 2H), 1.43 (t, J = 7.3 Hz, 3H). |
| 314 | ** | ** | 467.0 | 1H NMR(400 MHz, DMSO-d6) δ 9.12 (s, 1H), 8.54 |
| | | | | (s, 1H), 8.19 (s, 1H), 8.16 (s, 1H), 7.36-7.26 (m, |
| | | | | 2H), 7.06-6.96 (m, 1H), 4.59 (d, J = 3.6 Hz, 1H), |
| | | | | 3.96 (s, 2H), 3.91 (s, 1H), 3.69 (d, J = 15.7 Hz, 2H), |
| | | | | 2.08 (d, J = 4.7 Hz, 2H), 1.85 (d, J = 9.0 Hz, 2H). |
| 315 | * | *** | 385.2 | 1H NMR(400 MHz, DMSO-d6) δ 8.02 (d, J = 6.1 Hz, |
| | | | | 1H), 7.73 (s, 1H), 5.20 (s, 1H), 4.62-4.54 (m, 2H), |
| | | | | 3.89 (s, 3H), 3.79 (ddd, J = 27.5, 17.5, 11.7 Hz, 2H), |
| | | | | 3.53 (dd, J = 12.1, 5.0 Hz, 1H), 3.45-3.37 (m, 1H), |
| | | | | 3.28-3.21 (m, 1H), 3.09 (d, J = 12.1 Hz, 1H), 2.92- |
| | | | | 2.83 (m, 1H), 2.74 (dd, J = 12.3, 6.3 Hz, 1H), 2.19- |
| | | | | 2.08 (m, 4H), 1.99 (s, 3H), 1.97-1.91 (m, 1H). |
| 316 | *** | ** | 480.3 | 1H NMR(400 MHz, DMSO-d6) δ 8.26 (s, 1H), 8.03 |
| | | | | (s, 1H), 7.31 (t, J = 8.6 Hz, 1H), 6.83-6.74 (m, 2H), |
| | | | | 4.60 (d, J = 15.4 Hz, 2H), 3.90 (d, J = 1.8 Hz, 3H), |
| | | | | 3.83-3.74 (m, 5H), 3.52 (s, 2H), 3.04 (s, 4H), 2.88 |
| | | | | (t, J = 5.5 Hz, 1H), 2.75 (t, J = 5.5 Hz, 1H), 2.55 (s, |
| | | | | 4H), 2.11 (d, J = 6.0 Hz, 3H). |
| 317 | ** | ** | 462.4 | 1H NMR(400 MHz, DMSO-d6) δ 8.26 (s, 1H), 8.03 |
| | | | | (d, J = 1.2 Hz, 1H), 7.23 (d, J = 8.6 Hz, 2H), 6.88 (d, |
| | | | | J = 8.7 Hz, 2H), 4.61 (d, J = 15.4 Hz, 2H), 3.90 (d, J = |
| | | | | 1.8 Hz, 3H), 3.81-3.75 (m, 2H), 3.73 (s, 3H), 3.47 |
| | | | | (s, 2H), 3.04 (s, 4H), 2.88 (t, J = 5.8 Hz, 1H), 2.75 (t, |
| | | | | J = 5.7 Hz, 1H), 2.51(S, 4H), 2.11 (d, J = 6.0 Hz, 3H). |
| 318 | * | ** | 480.3 | 1H NMR(400 MHz, DMSO-d6) δ 8.27 (s, 1H), 8.03 |
| | | | | (s, 1H), 7.09 (p, J = 8.0 Hz, 2H), 7.03-6.94 (m, 1H), |
| | | | | 4.61 (d, J = 15.5 Hz, 2H), 3.91 (s, 3H), 3.81-3.75 |
| | | | | (m, 2H), 3.60 (s, 2H), 3.05 (s, 4H), 2.89 (t, J = 5.6 Hz, |
| | | | | 1H), 2.76 (t, J = 5.6 Hz, 1H), 2.58 (s, 4H), 2.12 (d, J = |
| | | | | 5.9 Hz, 3H). |
| 319 | ** | ** | 480.4 | 1H NMR(400 MHz, DMSO-d6) δ 8.27 (s, 1H), 8.04 |
| | | | | (d, J = 1.4 Hz, 1H), 7.19-7.07 (m, 3H), 4.62 (d, J = |
| | | | | 15.5 Hz, 2H), 3.91 (d, J = 1.7 Hz, 3H), 3.86-3.81 |
| | | | | (m, 3H), 3.81-3.76 (m, 2H), 3.49 (s, 2H), 3.06 (s, |
| | | | | 4H), 2.89 (t, J = 5.6 Hz, 1H), 2.76 (t, J = 5.7 Hz, 1H), |
| | | | | 2.58-2.47 (m, 4H), 2.12 (d, J = 6.0 Hz, 3H). |
| 320 | ** | ** | 468.1 | 1H NMR(400 MHz, DMSO-d6) δ 8.28 (s, 1H), 8.04 |
| | | | | (s, 1H), 7.38-7.31 (m, 1H), 7.26 (d, J = 5.5 Hz, 1H), |
| | | | | 7.20 (dd, J = 13.1, 7.5 Hz, 1H), 4.61 (d, J = 15.6 Hz, |
| | | | | 2H), 3.91 (s, 3H), 3.79 (d, J = 4.4 Hz, 1H), 3.66 (s, |
| | | | | 2H), 3.06 (s, 4H), 2.89 (s, 1H), 2.76 (s, 1H), 2.60 (s, |
| | | | | 4H), 2.49-2.41 (m, 2H), 2.12 (d, J = 5.9 Hz, 3H). |
| 321 | ** | ** | 468.2 | 1H NMR(400 MHz, DMSO-d6) δ 8.27 (s, 1H), 8.04 |
| | | | | (d, J = 1.4 Hz, 1H), 7.38 (dt, J = 10.9, 8.5 Hz, 2H), |
| | | | | 7.19 (d, J = 4.0 Hz, 1H), 4.61 (d, J = 15.5 Hz, 2H), |
| | | | | 3.90 (d, J = 1.6 Hz, 3H), 3.88-3.57 (m, 2H), 3.55 (s, |
| | | | | 2H), 3.06 (s, 4H), 2.89 (t, J = 5.6 Hz, 1H), 2.76 (t, J = |
| | | | | 5.7 Hz, 1H), 2.55 (s, 4H), 2.12 (d, J = 6.0 Hz, 3H). |
| 322 | * | ** | 492.4 | 1H NMR(400 MHz, DMSO-d6) δ 8.29 (s, 1H), 8.06 |
| | | | | (s, 1H), 7.25 (d, J = 8.3 Hz, 1H), 6.63-6.49 (m, 2H), |
| | | | | 4.61 (d, J = 15.0 Hz, 2H), 3.91 (s, 3H), 3.84-3.73 |
| | | | | (m, 8H), 3.58 (s, 2H), 3.08 (s, 4H), 2.89 (t, J = 5.5 Hz, |
| | | | | 1H), 2.76 (t, J = 5.3 Hz, 1H), 2.15-2.06 (m, 3H). |
| 323 | ** | ** | 451.2 | 1H NMR(400 MHz, DMSO-d6) δ 8.01 (d, J = 5.4 Hz, |
| | | | | 1H), 7.73 (s, 1H), 7.23-7.09 (m, 3H), 6.97-6.90 |
| | | | | (m, 1H), 4.57 (d, J = 16.0 Hz, 2H), 4.04 (p, J = 9.6 |
| | | | | Hz, 2H), 3.87 (s, 3H), 3.76 (qd, J = 12.8, 6.1 Hz, 2H), |
| | | | | 3.37 (dd, J = 6.9, 2.7 Hz, 2H), 3.27 (d, J = 7.2 Hz, |
| | | | | 1H), 3.26-3.16 (m, 1H), 2.85 (s, 1H), 2.72 (d, J = |
| | | | | 7.4 Hz, 1H), 2.66 (dd, J = 13.9, 7.0 Hz, 1H), 2.11 (d, |
| | | | | J = 8.2 Hz, 3H), 2.05 (dd, J = 11.9, 5.4 Hz, 1H). |
| 324 | ** | ** | 451.2 | 1H NMR(400 MHz, DMSO-d6) δ 8.02 (d, J = 5.6 Hz, |
| | | | | 1H), 7.73 (s, 1H), 7.29 (dd, J = 15.5, 8.1 Hz, 1H), |
| | | | | 6.77 (ddd, J = 17.1, 12.2, 6.8 Hz, 3H), 4.57 (d, J = |
| | | | | 15.9 Hz, 2H), 3.98 (p, J = 9.9 Hz, 2H), 3.87 (s, 3H), |
| | | | | 3.77 (tt, J = 13.4, 6.8 Hz, 2H), 3.38 (s, 1H), 3.27 (d, |
| | | | | J = 9.5 Hz, 2H), 3.17 (dd, J = 10.8, 6.9 Hz, 1H), 2.85 |
| | | | | (s, 1H), 2.73 (s, 1H), 2.69-2.60 (m, 1H), 2.11 (d, J = |
| | | | | 8.4 Hz, 3H), 2.05 (dd, J = 12.5, 6.4 Hz, 1H), 1.71 (dt, |
| | | | | J = 19.8, 7.6 Hz, 1H). |
| 325 | ** | ** | 451.2 | 1H NMR(400 MHz, DMSO-d6) δ 8.01 (d, J = 5.7 Hz, |
| | | | | 1H), 7.73 (s, 1H), 7.10 (t, J = 8.8 Hz, 2H), 6.98-6.90 |
| | | | | (m, 2H), 4.57 (d, J = 15.7 Hz, 2H), 3.95 (dd, J = 12.8, |
| | | | | 8.2 Hz, 2H), 3.88 (d, J = 11.5 Hz, 3H), 3.82-3.71 |
| | | | | (m, 2H), 3.36 (dd, J = 9.2, 1.6 Hz, 1H), 3.27 (s, 1H), |
| | | | | 3.23 (d, J = 5.3 Hz, 1H), 3.17 (dd, J = 10.7, 6.8 Hz, |
| | | | | 1H), 2.85 (s, 1H), 2.73 (s, 1H), 2.69-2.58 (m, 1H), |
| | | | | 2.11 (d, J = 8.5 Hz, 3H), 2.04 (dd, J = 12.3, 5.4 Hz, |
| | | | | 1H), 1.70 (td, J = 14.8, 7.2 Hz, 1H). |
| 326 | ** | ** | 508.5 | 1H NMR(400 MHz, DMSO-d6) δ 7.73 (d, J = 7.6 Hz, |
| | | | | 1H), 7.38-7.20 (overlapped, m, 3H), 7.05-6.92 (m, |
| | | | | 1H), 4.64-4.60 (m, 2H), 4.59-4.56 (m, 2H), 4.52- |
| | | | | 4.41 (m, 1H), 3.85-3.73 (m, 2H), 3.47-3.38 (m, 3H), |
| | | | | 3.30-3.23 (m, 1H), 3.04-2.94 (m, 2H), 2.94-2.87 |
| | | | | (m, 1H), 2.81-2.75 (m, 1H), 2.13 (s, 1.5H), 2.10 (s, |
| | | | | 1.5H), 1.95-1.80 (m, 2H), 1.65-1.52 (m, 2H). |
| 327 | ** | ** | 395.4 | 1H NMR (400 MHz, DMSO-d6) δ 9.07 (d, J = 0.5 |
| | | | | Hz, 1H), 8.53 (d, J = 5.7 Hz, 1H), 8.29 (s, 1H), 7.93 |
| | | | | (d, J = 0.6 Hz, 1H), 7.58 (dd, J = 5.7, 0.6 Hz, 1H), |
| | | | | 7.40-7.30 (m, 1H), 7.05-6.96 (m, 2H), 5.11-5.04 |
| | | | | (m, 1H), 4.52 (dd, J = 11.1, 6.4 Hz, 2H), 4.08 (dd, J = |
| | | | | 11.1, 4.0 Hz, 2H), 3.94 (s, 3H) |
| 328 | *** | ** | 457.3 | 1H NMR (400 MHz, DMSO-d6) δ 8.50 (s, 1H), 8.31 |
| | | | | (d, J = 5.6 Hz, 1H), 8.15 (s, 1H), 7.61 (d, J = 5.6 Hz, |
| | | | | 1H), 7.36-7.24 (m, 2H), 7.05-6.97 (m, 1H), 4.62- |
| | | | | 4.53 (m, 1H), 3.97 (s, 3H), 3.75-3.65 (m, 2H), 3.33- |
| | | | | 3.27 (overlapping m, 2H), 2.11-2.01 (m, 2H), 1.89- |
| | | | | 1.76 (m, 2H). |
| 329 | **** | *** | 496.4 | 1H NMR(400 MHz, DMSO-d6) δ 8.02 (s, 1H), 7.88 |
| | | | | (s, 1H), 7.33-7.22 (overlapped, m, 2H), 7.21-7.12 |
| | | | | (m, 1H), 7.04-6.93 (m, 1H), 4.66-4.57 (m, 2H), |
| | | | | 4.54-4.43 (m, 1H), 3.86-3.73 (m, 2H), 3.54-3.40 |
| | | | | (m, 2H), 3.10-2.96 (m, 2H), 2.91 (t, J = 5.6 Hz, 1H), |
| | | | | 2.83 (d, J = 4.6 Hz, 3H), 2.78 (t, J = 6.1 Hz, 1H), 2.13 |
| | | | | (s, 1.5H), 2.10 (s, 1.5H), 1.97-1.82 (m, 2H), 1.67- |
| | | | | 1.49 (m, 2H). |
| 330 | *** | ** | 466.3 | 1H NMR(400 MHz, DMSO-d6) δ 9.10 (d, J = 0.6 Hz, |
| | | | | 1H), 8.54 (s, 1H), 8.19 (dd, J = 9.3, 6.5 Hz, 3H), 7.74 |
| | | | | (s, 1H), 7.36-7.26 (m, 2H), 7.05-6.98 (m, 1H), |
| | | | | 4.59 (s, 1H), 3.97 (s, 3H), 3.69 (s, 2H), 3.29 (s, 1H), |
| | | | | 2.07 (s, 2H), 1.85 (d, J = 9.0 Hz, 2H). |
| 331 | ** | ** | 487.2 | 1H NMR(400 MHz, DMSO-d6) δ 8.33 (s, 1H), 8.04 |
| | | | | (s, 1H), 7.40-7.28 (m, 2H), 7.07-7.00 (m, 1H), |
| | | | | 5.12-4.92 (m, 1H), 4.59 (dt, J = 16.8, 14.0 Hz, 3H), |
| | | | | 3.93 (d, J = 1.7 Hz, 3H), 3.84-3.77 (m, 2H), 3.70 |
| | | | | (td, J = 11.4, 3.4 Hz, 1H), 3.28 (s, 1H), 3.06 (ddd, J = |
| | | | | 12.2, 9.0, 6.3 Hz, 1H), 2.91 (dd, J = 21.3, 8.5 Hz, 2H), |
| | | | | 2.79 (t, J = 5.5 Hz, 1H), 2.13 (d, J = 5.8 Hz, 4H), 1.86- |
| | | | | 1.74 (m, 1H). |
| 332 | ** | ** | 411.1 | 1H NMR (400 MHz, DMSO-d6) δ 9.13 (s, 1H), 8.57 |
| | | | | (d, J = 5.7 Hz, 1H), 8.47 (s, 1H), 8.13 (s, 1H), 7.64 (d, |
| | | | | J = 5.3 Hz, 1H), 7.39-7.28 (m, 2H), 7.07-6.99 (m, |
| | | | | 1H), 5.15-4.98 (m, 1H), 4.72 (dd, J = 22.5, 8.1 Hz, |
| | | | | 1H), 3.99-3.82 (m, 5H), 3.54 (dd, J = 28.8, 13.5 Hz, |
| | | | | 1H), 3.22 (t, J = 10.5 Hz, 1H), 2.24-2.12 (m, 1H), |
| | | | | 2.03-1.94 (m, 1H). |
| 333 | ** | * | 535.3 | 1H NMR(400 MHz, DMSO-d6) δ 8.24 (s, 1H), 8.01 |
| | | | | (s, 1H), 7.42-7.26 (m, 8H), 7.06-6.99 (m, 1H), |
| | | | | 5.07 (d, J = 51.3 Hz, 1H), 4.69 (d, J = 21.0 Hz, 1H), |
| | | | | 3.88 (s, 3H), 3.72 (s, 2H), 3.59-3.47 (m, 3H), 3.22 |
| | | | | (d, J = 13.3 Hz, 1H), 2.99-2.89 (m, 1H), 2.87-2.78 |
| | | | | (m, 4H), 2.17-2.06 (m, 1H), 1.95 (d, J = 3.4 Hz, 1H). |
| 334 | ** | ** | 487.2 | 1H NMR(400 MHz, DMSO-d6) δ 8.29 (s, 1H), 8.05 |
| | | | | (s, 1H), 7.39-7.28 (m, 2H), 7.06-7.00 (m, 1H), |
| | | | | 5.08 (d, J = 54.2 Hz, 1H), 4.64 (dd, J = 26.5, 11.5 Hz, |
| | | | | 3H), 3.91 (d, J = 1.6 Hz, 3H), 3.87-3.72 (m, 2H), |
| | | | | 3.56 (d, J = 6.1 Hz, 1H), 3.29-3.20 (m, 1H), 2.94 |
| | | | | (dd, J = 20.4, 9.8 Hz, 2H), 2.81-2.74 (m, 2H), 2.12 |
| | | | | (d, J = 5.3 Hz, 4H), 1.96 (d, J = 4.2 Hz, 1H). |
| 335 | * | ** | 433.3 | 1H NMR(400 MHz, DMSO-d6) δ 8.32 (s, 1H), 8.07 |
| | | | | (s, 0.5H), 7.99 (d, J = 7.3 Hz, 0.38H), 7.77 (s, 0.3H), |
| | | | | 7.26 (t, J = 7.9 Hz, 2H), 6.94 (dt, J = 14.6, 7.9 Hz, |
| | | | | 3H), 4.74-4.48 (m, 3H), 4.19 (dd, J = 10.0, 3.8 Hz, |
| | | | | 0.4H), 4.03 (dd, J = 14.2, 7.1 Hz, 0.3H), 3.98-3.87 |
| | | | | (m, 4H), 3.85-3.70 (m, 2H), 3.65-3.56 (m, 2H), |
| | | | | 3.44-3.36 (m, 0.78H), 3.18 (d, J = 12.2 Hz, 1H), |
| | | | | 3.00-2.82 (m, 3.4H), 2.69 (dd, J = 15.4, 4.5 Hz, 1H), |
| | | | | 2.22-2.01 (m, 4H), 1.86 (d, J = 18.5 Hz, 1.5H), 1.64 |
| | | | | (d, J = 7.7 Hz, 1.7H). |
| 336 | * | *** | 451.3 | 1H NMR(400 MHz, DMSO-d6) δ 8.33 (s, 0.5H), 8.06 |
| | | | | (d, J = 2.7 Hz, 0.5H), 8.00 (d, J = 6.4 Hz, 0.5H), 7.77 |
| | | | | (s, 0.5H), 7.34-7.24 (m, 1H), 7.19 (ddd, J = 11.9, |
| | | | | 7.9, 1.6 Hz, 1H), 7.10 (t, J = 7.8 Hz, 1H), 6.93 (ddd, |
| | | | | J = 14.4, 10.3, 4.6 Hz, 1H), 4.75 (s, 0.5H), 4.66 (d, J = |
| | | | | 9.8 Hz, 0.5H), 4.62 (s, 0.5H), 4.62-4.56 (m, 1H), |
| | | | | 4.27 (dd, J = 10.1, 3.4 Hz, 0.5H), 4.06 (dd, J = 10.0, |
| | | | | 6.6 Hz, 0.5H), 3.97-3.86 (m, 3H), 3.81 (d, J = 5.0 |
| | | | | Hz, 0.5H), 3.77 (s, 0.5H), 3.71 (d, J = 8.4 Hz, 1H), |
| | | | | 3.68-3.59 (m, 0.3H), 3.54 (d, J = 12.2 Hz, 1H), 3.08 |
| | | | | (s, 0.5H), 3.05-2.97 (m, 0.6H), 2.91 (dd, J = 11.4, |
| | | | | 6.0 Hz, 1H), 2.80 (d, J = 18.4 Hz, 1H), 2.73-2.62 |
| | | | | (m, 0.1H), 2.23-2.15 (m, 0.5H), 2.15-2.07 (m, 3H), |
| | | | | 2.01 (s, 0.6H), 1.91 (s, 2H), 1.66 (dd, J = 16.8, 9.1 Hz, 2H). |
| 337 | * | ** | 451.3 | 1H NMR(400 MHz, DMSO-d6) δ 8.30 (s, 0.5H), 8.06 |
| | | | | (d, J = 3.6 Hz, 0.5H), 8.01 (d, J = 6.9 Hz, 0.5H), 7.77 |
| | | | | (s, 0.5H), 7.29 (dd, J = 15.6, 7.4 Hz, 1H), 6.90 (d, J = |
| | | | | 11.5 Hz, 2H), 6.83 (t, J = 7.6 Hz, 1H), 6.77-6.70 (m, |
| | | | | 1H), 4.74 (s, 0.5H), 4.70-4.49 (m, 2.5H), 4.22 (dd, |
| | | | | J = 10.1, 3.8 Hz, 0.5H), 4.01-3.93 (m, 0.6H), 3.90 (d, |
| | | | | J = 10.5 Hz, 3H), 3.78 (dd, J = 22.4, 17.6 Hz, 2H), |
| | | | | 3.60 (d, J = 12.9 Hz, 0.8H), 3.38 (dd, J = 22.4, 9.5 Hz, |
| | | | | 1H), 3.18 (d, J = 13.2 Hz, 0.6H), 3.01-2.83 (m, 2H), |
| | | | | 2.79 (s, 0.6H), 2.20-2.03 (m, 4.6H), 1.85 (d, J = 11.9 |
| | | | | Hz, 2H), 1.63 (s, 2H). |
| 338 | * | ** | 451.4 | 1H NMR(400 MHz, DMSO-d6) δ 8.30 (s, 0.7H), 8.06 |
| | | | | (s, 0.6H), 8.01 (d, J = 7.5 Hz, 0.4H), 7.76 (s, 0.3H), |
| | | | | 7.09 (t, J = 8.8 Hz, 2H), 7.04-6.94 (m, 2H), 4.59 |
| | | | | (dd, J = 20.3, 14.4 Hz, 3H), 4.17 (dd, J = 9.9, 3.7 Hz, |
| | | | | 0.4H), 3.90 (d, J = 9.8 Hz, 4H), 3.79 (ddd, J = 19.4, |
| | | | | 13.8, 7.7 Hz, 3.6H), 3.57 (d, J = 11.8 Hz, 0.8H), 3.44- |
| | | | | 3.36 (m, 0.4H), 3.17 (d, J = 12.9 Hz, 0.7H), 2.87 |
| | | | | (ddd, J = 36.2, 25.4, 8.4 Hz, 4H), 2.20-1.99 (m, 4H), |
| | | | | 1.85 (d, J = 12.4 Hz, 1.5H), 1.63 (d, J = 6.3 Hz, 1.6H). |
| 339 | * | *** | 469.4 | 1H NMR(400 MHz, DMSO-d6) δ 8.31 (s, 0.6H), 8.04 |
| | | | | (dd, J = 17.8, 4.8 Hz, 1H), 7.77 (s, 0.37H), 7.32- |
| | | | | 7.20 (m, 2H), 6.75 (ddd, J = 17.5, 11.8, 6.0 Hz, 1H), |
| | | | | 4.81 (d, J = 3.6 Hz, 0.7H), 4.72-4.52 (m, 3H), 4.32- |
| | | | | 4.26 (m, 0.4H), 4.12-4.06 (m, 0.4H), 3.91 (t, J = |
| | | | | 14.4 Hz, 3H), 3.86-3.66 (m, 2H), 3.55 (d, J = 10.7 |
| | | | | Hz, 0.7H), 3.44-3.35 (m, 0.6H), 3.05 (dd, J = 12.4, |
| | | | | 6.9 Hz, 1.3H), 3.00-2.85 (m, 1.6H), 2.86-2.72 (m, |
| | | | | 1.4H), 2.19-2.08 (m, 3.5H), 2.02 (s, 0.7H), 1.88 (s, |
| | | | | 1.5H), 1.77-1.56 (m, 1.7H). |
| 340 | ** | * | 437.3 | 1H NMR(400 MHz, DMSO-d6) δ 8.04 (d, J = 5.1 Hz, |
| | | | | 1H), 7.75 (d, J = 0.5 Hz, 1H), 7.18 (dddd, J = 24.6, |
| | | | | 22.9, 11.9, 4.7 Hz, 3H), 6.99-6.92 (m, 1H), 5.09 (s, |
| | | | | 1H), 4.63-4.53 (m, 2H), 3.87 (d, J = 1.4 Hz, 3H), |
| | | | | 3.85-3.68 (m, 2H), 3.65-3.57 (m, 1H), 3.50 (dd, |
| | | | | J = 17.5, 8.8 Hz, 2H), 2.91-2.70 (m, 2H), 2.27-2.18 |
| | | | | (m, 1H), 2.11 (d, J = 8.2 Hz, 3H), 2.07 (s, 1H). |
| 341 | ** | ** | 437.2 | 1H NMR(400 MHz, DMSO-d6) δ 8.03 (d, J = 5.5 Hz, |
| | | | | 1H), 7.74 (s, 1H), 7.14-7.07 (m, 2H), 6.96-6.91 |
| | | | | (m, 2H), 5.00 (s, 1H), 4.62-4.54 (m, 2H), 3.87 (s, |
| | | | | 3H), 3.85-3.67 (m, 2H), 3.63-3.56 (m, 1H), 3.53- |
| | | | | 3.43 (m, 1H), 2.92-2.82 (m, 1H), 2.76-2.70 (m, |
| | | | | 2H), 2.25-2.15 (m, 1H), 2.11 (d, J = 8.7 Hz, 3H), |
| | | | | 2.06 (d, J = 11.2 Hz, 1H). |
| 342 | ** | *** | 437.2 | 1H NMR(400 MHz, DMSO-d6) δ 8.04 (d, J = 5.5 Hz, |
| | | | | 1H), 7.75 (s, 1H), 7.30-7.13 (m, 1H), 6.78 (ddd, J = |
| | | | | 11.9, 8.5, 3.1 Hz, 1H), 5.13 (s, 1H), 4.63-4.54 (m, |
| | | | | 2H), 3.87 (s, 3H), 3.85-3.69 (m, 2H), 3.61 (d, J = |
| | | | | 12.3 Hz, 1H), 3.54-3.43 (m, 2H), 2.91-2.70 (m, |
| | | | | 2H), 2.29-2.19 (m, 1H), 2.11 (d, J = 8.2 Hz, 3H), |
| | | | | 2.07 (s, 1H). |
| 343 | ** | ** | 486.4 | 1H NMR(400 MHz, DMSO-d6) δ 8.26 (s, 1H), 8.02 |
| | | | | (s, 1H), 7.24-7.16 (m, 2H), 4.60 (d, J = 15.3 Hz, |
| | | | | 2H), 3.90 (d, J = 1.5 Hz, 3H), 3.80-3.74 (m, 2H), |
| | | | | 3.60 (s, 2H), 3.02 (s, 4H), 2.88 (t, J = 5.7 Hz, 1H), |
| | | | | 2.74 (t, J = 5.9 Hz, 1H), 2.57 (s, 4H), 2.11 (d, J = 6.0 |
| | | | | Hz, 3H). |
| 344 | * | ** | 463.2 | 1H NMR(400 MHz, DMSO-d6) δ 8.27 (s, 1H), 8.20 |
| | | | | (dd, J = 2.4, 1.1 Hz, 1H), 8.03 (d, J = 1.3 Hz, 1H), |
| | | | | 7.37 (t, J = 1.9 Hz, 2H), 4.61 (d, J = 15.6 Hz, 2H), |
| | | | | 3.91 (d, J = 1.9 Hz, 3H), 3.83-3.75 (m, 5H), 3.61 (s, |
| | | | | 2H), 3.06 (s, 4H), 2.89 (t, J = 5.7 Hz, 1H), 2.76 (t, J = |
| | | | | 5.7 Hz, 1H), 2.58 (s, 4H), 2.12 (d, J = 5.8 Hz, 3H). |
| 345 | ** | ** | 463.2 | 1H NMR(400 MHz, DMSO-d6) δ 8.26 (s, 1H), 8.07 |
| | | | | (d, J = 2.0 Hz, 1H), 8.03 (d, J = 1.3 Hz, 1H), 7.65 (dd, |
| | | | | J = 8.5, 2.4 Hz, 1H), 6.79 (d, J = 8.4 Hz, 1H), 4.61 (d, |
| | | | | J = 15.5 Hz, 2H), 3.91 (d, J = 1.7 Hz, 3H), 3.83 (s, |
| | | | | 3H), 3.82-3.75 (m, 2H), 3.50 (s, 2H), 3.05 (s, 4H), |
| | | | | 2.88 (t, J = 5.5 Hz, 1H), 2.75 (t, J = 5.6 Hz, 1H), 2.53 |
| | | | | (s, 4H), 2.11 (d, J = 5.9 Hz, 3H). |
| 346 | ** | *** | 468.4 | 1H NMR(400 MHz, DMSO-d6) δ 8.26 (s, 1H), 8.01 |
| | | | | (s, 1H), 7.47-7.37 (m, 1H), 7.10 (dd, J = 14.0, 6.0 |
| | | | | Hz, 2H), 4.60 (d, J = 15.2 Hz, 2H), 3.90 (d, J = 1.6 |
| | | | | Hz, 3H), 3.77 (dd, J = 10.3, 5.6 Hz, 2H), 3.64 (s, 2H), |
| | | | | 3.02 (s, 4H), 2.87 (t, J = 5.7 Hz, 1H), 2.74 (t, J = 5.9 |
| | | | | Hz, 1H), 2.58 (s, 4H), 2.11 (d, J = 6.0 Hz, 3H). |
| 347 | ** | ** | 464.2 | 1H NMR(400 MHz, DMSO-d6) δ 8.28 (s, 1H), 8.06 |
| | | | | (d, J = 1.8 Hz, 1H), 7.27 (dd, J = 8.4, 6.3 Hz, 1H), |
| | | | | 7.02 (dd, J = 10.1, 2.6 Hz, 1H), 6.94 (td, J = 8.5, 2.6 |
| | | | | Hz, 1H), 4.61 (d, J = 15.6 Hz, 2H), 3.91 (d, J = 1.9 |
| | | | | Hz, 3H), 3.83-3.73 (m, 2H), 3.49 (s, 2H), 3.03 (s, |
| | | | | 4H), 2.88 (t, J = 5.8 Hz, 1H), 2.75 (t, J = 5.8 Hz, 1H), |
| | | | | 2.54 (s, 4H), 2.34(s, 3H) 2.10 (dd, J = 13.7, 5.1 Hz, 3H). |
| 348 | *** | ** | 484.2 | 1H NMR(400 MHz, DMSO-d6) δ 8.28 (s, 1H), 8.04 |
| | | | | (s, 1H), 7.48 (t, J = 8.2 Hz, 1H), 7.41 (dd, J = 9.9, 2.0 |
| | | | | Hz, 1H), 7.29 (dd, J = 8.2, 1.9 Hz, 1H), 4.61 (d, J = |
| | | | | 15.5 Hz, 2H), 3.91 (s, 3H), 3.84-3.71 (m, 2H), 3.60 |
| | | | | (s, 2H), 3.06 (s, 4H), 2.90 (m, 1H), 2.76 (t, J = 5.5 Hz, |
| | | | | 2H), 2.58 (s, 4H), 2.16-2.07 (d, J = 6.0 Hz, 3H). |
| 349 | **** | ** | 484.2 | 1H NMR(400 MHz, DMSO-d6) δ 8.27 (s, 1H), 8.04 |
| | | | | (s, 1H), 7.49 (dd, J = 6.2, 2.7 Hz, 1H), 7.42-7.36 (m, |
| | | | | 1H), 7.25 (t, J = 9.2 Hz, 1H), 4.61 (d, J = 15.5 Hz, |
| | | | | 2H), 3.91 (d, J = 1.6 Hz, 3H), 3.90 (s, 2H), 3.80 (m, |
| | | | | 2H), 3.61 (s, 2H), 3.06 (s, 4H), 2.89 (t, J = 5.6 Hz, |
| | | | | 1H), 2.75 (t, J = 5.7 Hz, 1H), 2.59 (s, 4H), 2.12 (d, J = |
| | | | | 5.9 Hz, 3H). |
| 350 | ** | ** | 500.2 | 1H NMR(400 MHz, DMSO-d6) δ 8.29 (s, 1H), 8.05 |
| | | | | (d, J = 1.7 Hz, 1H), 7.60 (d, J = 2.1 Hz, 1H), 7.55 (d, |
| | | | | J = 8.3 Hz, 1H), 7.42 (dd, J = 8.3, 2.1 Hz, 1H), 4.62 (d, |
| | | | | J = 15.6 Hz, 2H), 3.91 (s, 3H), 3.79 (dd, J = 10.1, 5.5 |
| | | | | Hz, 2H), 3.64 (s, 2H), 3.07 (s, 4H), 2.89 (t, J = 5.7 Hz, |
| | | | | 1H), 2.76 (t, J = 5.7 Hz, 1H), 2.62 (s, 4H), 2.12 (d, J = |
| | | | | 5.6 Hz, 3H). |
| 351 | ** | ** | 457.2 | 1H NMR(400 MHz, DMSO-d6) δ 8.30 (s, 1H), 8.06 |
| | | | | (d, J = 2.1 Hz, 1H), 7.83 (dd, J = 7.7, 0.9 Hz, 1H), |
| | | | | 7.69 (td, J = 7.6, 1.2 Hz, 1H), 7.62 (d, J = 7.3 Hz, |
| | | | | 1H), 7.48 (td, J = 7.6, 1.2 Hz, 1H), 4.62 (d, J = 15.8 |
| | | | | Hz, 2H), 3.92 (s, 3H), 3.78 (dd, J = 9.7, 3.9 Hz, 2H), |
| | | | | 3.75 (s, 2H), 3.07 (s, 4H), 2.90 (t, J = 5.8 Hz, 1H), |
| | | | | 2.76 (t, J = 5.7 Hz, 1H), 2.62 (s, 4H), 2.12 (d, J = 5.3 |
| | | | | Hz, 3H). |
| 352 | *** | ** | 468.2 | 1H NMR(400 MHz, DMSO-d6) δ 8.27 (s, 1H), 8.04 |
| | | | | (s, 1H), 7.48 (dd, J = 15.5, 8.5 Hz, 1H), 7.22 (td, J = |
| | | | | 10.2, 2.5 Hz, 1H), 7.08 (td, J = 8.5, 2.2 Hz, 1H), 4.61 |
| | | | | (d, J = 15.3 Hz, 2H), 3.91 (d, J = 1.6 Hz, 3H), 3.79 |
| | | | | (dd, J = 10.1, 5.5 Hz, 2H), 3.59 (s, 2H), 3.05 (s, 4H), |
| | | | | 2.89 (t, J = 5.6 Hz, 1H), 2.76 (t, J = 5.7 Hz, 1H), 2.57 |
| | | | | (s, 4H), 2.12 (d, J = 5.9 Hz, 3H). |
| 353 | ** | ** | 468.2 | 1H NMR(400 MHz, DMSO-d6) δ 8.28 (s, 1H), 8.05 |
| | | | | (d, J = 1.6 Hz, 1H), 7.15-7.03 (m, 3H), 4.62 (d, J = |
| | | | | 15.4 Hz, 2H), 3.91 (d, J = 1.7 Hz, 3H), 3.79 (dd, J = |
| | | | | 10.0, 5.6 Hz, 2H), 3.60 (s, 2H), 3.08 (s, 4H), 2.90 (t, |
| | | | | J = 5.7 Hz, 1H), 2.76 (t, J = 5.7 Hz, 1H), 2.57 (s, 4H), |
| | | | | 2.12 (d, J = 5.9 Hz, 3H). |
| 354 | ** | ** | 486.4 | 1H NMR(400 MHz, DMSO-d6) δ 8.27 (s, 1H), 8.02 |
| | | | | (s, 1H), 7.49 (ddd, J = 19.0, 9.5, 5.1 Hz, 1H), 7.16 (t, |
| | | | | J = 7.3 Hz, 1H), 4.60 (d, J = 15.3 Hz, 2H), 3.90 (d, J = |
| | | | | 1.5 Hz, 3H), 3.77 (dd, J = 10.2, 5.6 Hz, 2H), 3.68 (s, |
| | | | | 2H), 3.03 (s, 4H), 2.88 (t, J = 5.7 Hz, 1H), 2.74 (t, J = |
| | | | | 5.7 Hz, 1H), 2.59 (bs, 4H), 2.11 (d, J = 6.0 Hz, 3H). |
| 355 | ** | ** | 475.4 | 1H NMR(400 MHz, DMSO-d6) δ 8.29 (s, 1H), 8.05 |
| | | | | (d, J = 2.0 Hz, 1H), 7.83 (dd, J = 8.7, 2.7 Hz, 1H), |
| | | | | 7.65 (dd, J = 8.7, 5.6 Hz, 1H), 7.57 (td, J = 8.6, 2.7 |
| | | | | Hz, 1H), 4.61 (d, J = 15.8 Hz, 2H), 3.91 (d, J = 1.7 |
| | | | | Hz, 3H), 3.82-3.75 (m, 2H), 3.72 (s, 2H), 3.06 (s, |
| | | | | 4H), 2.89 (t, J = 5.8 Hz, 1H), 2.76 (t, J = 5.7 Hz, 1H), |
| | | | | 2.61 (s, 4H), 2.12 (d, J = 5.4 Hz, 3H). |
| 356 | ** | ** | 475.4 | 1H NMR(400 MHz, DMSO-d6) δ 8.27 (s, 1H), 8.04 |
| | | | | (d, J = 1.3 Hz, 1H), 7.86-7.82 (m, 1H), 7.72-7.65 |
| | | | | (m, 2H), 4.61 (d, J = 15.5 Hz, 2H), 3.91 (d, J = 1.7 |
| | | | | Hz, 3H), 3.78 (dd, J = 10.0, 5.5 Hz, 2H), 3.69 (s, 2H), |
| | | | | 3.06 (s, 4H), 2.89 (t, J = 5.8 Hz, 1H), 2.75 (t, J = 5.7 |
| | | | | Hz, 1H), 2.60 (s, 4H), 2.12 (t, J = 5.9 Hz, 3H). |
| 357 | ** | ** | 475.4 | 1H NMR(400 MHz, DMSO-d6) δ 8.27 (s, 1H), 8.04 |
| | | | | (d, J = 1.4 Hz, 1H), 7.94 (dd, J = 6.7, 2.1 Hz, 1H), |
| | | | | 7.87 (ddd, J = 8.4, 4.8, 2.2 Hz, 1H), 7.45 (dd, J = 9.6, |
| | | | | 8.7 Hz, 1H), 4.61 (d, J = 15.5 Hz, 2H), 3.91 (d, J = |
| | | | | 1.7 Hz, 3H), 3.78 (dd, J = 10.0, 5.7 Hz, 2H), 3.65 (s, |
| | | | | 2H), 3.07 (s, 4H), 2.89 (t, J = 5.8 Hz, 1H), 2.75 (t, J = |
| | | | | 5.7 Hz, 1H), 2.60 (s, 4H), 2.12 (d, J = 5.9 Hz, 3H). |
| 358 | ** | ** | 516.4 | 1H NMR(400 MHz, DMSO-d6) δ 8.28 (d, J = 8.3 Hz, |
| | | | | 1H), 8.03 (s, 1H), 7.52-7.46 (m, 1H), 7.29 (s, 1H), |
| | | | | 7.11 (dd, J = 11.1, 2.0 Hz, 1H), 7.03 (dd, J = 8.4, 2.2 |
| | | | | Hz, 1H), 4.61 (d, J = 15.5 Hz, 2H), 3.91 (d, J = 1.7 |
| | | | | Hz, 3H), 3.82-3.75 (m, 2H), 3.59 (s, 2H), 3.05 (s, |
| | | | | 4H), 2.88 (t, J = 5.6 Hz, 1H), 2.75 (t, J = 5.7 Hz, 1H), |
| | | | | 2.57 (s, 4H), 2.11 (d, J = 5.9 Hz, 3H). |
| 359 | ** | ** | 516.4 | 1H NMR(400 MHz, DMSO-d6) δ 8.27 (s, 1H), 8.04 |
| | | | | (s, 1H), 7.46 (d, J = 8.5 Hz, 2H), 7.32 (d, J = 8.1 Hz, |
| | | | | 2H), 4.61 (d, J = 15.6 Hz, 2H), 3.90 (s, 3H), 3.83- |
| | | | | 3.72 (m, 2H), 3.59 (s, 2H), 3.06 (s, 4H), 2.89 (t, J = |
| | | | | 5.8 Hz, 1H), 2.80-2.71 (m, 1H), 2.55 (s, 4H), 2.19- |
| | | | | 2.08 (m, 3H). |
| 360 | *** | ** | 502.3 | 1H NMR(400 MHz, DMSO-d6) δ 8.27 (s, 1H), 8.05 |
| | | | | (s, 1H), 7.70 (d, J = 8.1 Hz, 1H), 7.57 (d, J = 8.0 Hz, |
| | | | | 2H), 4.61 (d, J = 15.5 Hz, 2H), 3.91 (d, J = 1.6 Hz, |
| | | | | 3H), 3.83-3.75 (m, 2H), 3.66 (s, 2H), 3.07 (s, 4H), |
| | | | | 2.89 (t, J = 5.8 Hz, 1H), 2.76 (t, J = 5.6 Hz, 1H), 2.57 |
| | | | | (s, 4H), 2.12 (d, J = 5.9 Hz, 3H). |
| 361 | ** | ** | 514.3 | 1H NMR(400 MHz, DMSO-d6) δ 8.27 (s, 1H), 8.04 |
| | | | | (s, 1H), 7.46 (d, J = 7.7 Hz, 1H), 7.10 (d, J = 11.7 Hz, |
| | | | | 1H), 4.61 (d, J = 15.4 Hz, 2H), 3.91 (d, J = 1.6 Hz, |
| | | | | 3H), 3.86 (s, 3H), 3.83-3.74 (m, 2H), 3.82-3.74 |
| | | | | (m, 2H), 3.54 (s, 2H), 3.05 (s, 4H), 2.89 (t, J = 5.8 Hz, |
| | | | | 1H), 2.76 (t, J = 5.6 Hz, 1H), 2.56 (s, 4H), 2.12 (d, J = |
| | | | | 6.0 Hz, 3H). |
| 362 | ** | ** | 494.4 | 1H NMR(400 MHz, DMSO-d6) δ 8.26 (s, 1H), 8.03 |
| | | | | (s, 1H), 7.29 (t, J = 8.8 Hz, 1H), 6.81-6.70 (m, 2H), |
| | | | | 4.60 (d, J = 15.3 Hz, 2H), 4.02 (q, J = 7.0 Hz, 2H), |
| | | | | 3.90 (s, 3H), 3.76 (dd, J = 12.7, 7.2 Hz, 2H), 3.52 (s, |
| | | | | 2H), 3.04 (bs, 4H), 2.88 (t, J = 5.7 Hz, 2H), 2.75 (t, |
| | | | | J = 5.6 Hz, 1H), 2.54 (bs, 4H), 2.11 (d, J = 5.9 Hz, 3H), |
| | | | | 1.31 (t, J = 7.0 Hz, 3H). |
| 363 | ** | ** | 433.3 | 1H NMR(400 MHz, DMSO-d6) δ 8.01 (d, J = 5.5 Hz, |
| | | | | 1H), 7.73 (s, 1H), 7.33-7.22 (m, 2H), 6.96-6.85 |
| | | | | (m, 3H), 4.57 (d, J = 15.6 Hz, 2H), 4.00-3.91 (m, |
| | | | | 2H), 3.86 (s, 3H), 3.77 (m, 2H), 3.40-3.34 (m, 1H), |
| | | | | 3.27-3.14 (m, 3H), 2.85 (bs, 1H), 2.73 (bs, 1H), 2.69- |
| | | | | 2.58 (m, 2H), 2.11 (d, J = 8.5 Hz, 3H), 2.03 (m, |
| | | | | 1H), 1.72 (m, 1H). |
| 364 | ** | ** | 469.2 | 1H NMR(400 MHz, DMSO-d6) δ 8.01 (d, J = 5.6 Hz, |
| | | | | 1H), 7.73 (s, 1H), 7.24 (m, 1H), 7.14 (m, 1H), 6.75 |
| | | | | (m, 1H), 4.57 (d, J = 16.0 Hz, 2H), 4.05 (m, 2H), |
| | | | | 3.87 (s, 3H), 3.76 (m, 2H), 3.39-3.35 (m, 1H), 3.28- |
| | | | | 3.14 (m, 3H), 2.85 (bs, 1H), 2.76-2.63 (m, 3H), |
| | | | | 2.11 (d, J = 8.2 Hz, 3H), 2.05 (m, 1H), 1.71 (m, 1H). |
| 365 | * | * | 482.2 | 1H NMR(400 MHz, DMSO-d6) δ 9.82 (s, 1H), 8.05 |
| | | | | (d, J = 5.6 Hz, 1H), 7.81-7.71 (m, 2H), 7.35-7.25 |
| | | | | (m, 1H), 7.05 (t, J = 8.6 Hz, 1H), 4.58 (d, J = 16.2 Hz, |
| | | | | 2H), 3.88 (s, 3H), 3.80 (dd, J = 15.0, 6.5 Hz, 2H), |
| | | | | 3.41 (m, 2H), 3.27-3.19 (m, 2H), 2.87 (m, 1H), 2.74 |
| | | | | (m, 1H), 2.20-2.03 (m, 6H). |
| 366 | *** | ** | 525.5 | 1H NMR(400 MHz, DMSO-d6) δ 8.40-8.27 (m, |
| | | | | 1H), 8.09-8.00 (m, 1H), 7.38-7.20 (m, 2H), 7.08- |
| | | | | 6.93 (m, 1H), 4.78-4.61 (m, 5H), 4.55-4.45 (m, |
| | | | | 1H), 4.38 (s, 1H), 4.32-4.16 (m, 3H), 3.85 (t, J = 5.8 |
| | | | | Hz, 1H, overlapping), 3.54 (t, J = 5.8 Hz, 1H, |
| | | | | overlapping), 3.36-3.24 (m, 2H, overlapping), 3.01- |
| | | | | 2.91 (m, 2H), 2.86-2.74 (m, 2H), 2.12-2.00 (m, |
| | | | | 2H), 1.90-1.76 (m, 2H), 1.45-1.34 (m, 3H). |
| 367 | ** | ** | 494.4 | 1H NMR(400 MHz, DMSO-d6) δ 10.69 (s, 1H), 8.91 |
| | | | | (s, 1H), 8.49 (s, 1H), 8.29 (s, 1H), 8.12 (s, 1H), 7.36- |
| | | | | 7.27 (m, 2H), 7.06-6.98 (m, 1H), 4.59-4.56 (m, |
| | | | | 1G), 4.26 (q, J = 7.2 Hz, 2H), 3.70-3.62 (m, 2H), |
| | | | | 3.27-3.23 (t, J = 9.6 Hz, 2H), 2.16 (s, 3H), 2.13- |
| | | | | 2.04 (m, 2H), 1.87-1.83 (m, 2H), 1.43 (t, J = 7.2 Hz, 3H). |
| 368 | **** | *** | 421.5 | 1H NMR (400 MHz, DMSO-d6) δ 9.09 (d, J = 0.4 |
| | | | | Hz, 1H), 8.53 (d, J = 5.7 Hz, 1H), 8.46 (s, 1H), 8.13 |
| | | | | (d, J = 0.4 Hz, 1H), 7.60 (dd, J = 5.7, 0.6 Hz, 1H), |
| | | | | 7.34 (td, J = 8.6, 7.0 Hz, 1H), 7.22-7.15 (m, 1H), |
| | | | | 7.03 (td, J = 8.4, 2.2 Hz, 1H), 3.95 (s, 3H), 3.83 (d, |
| | | | | J = 12.9 Hz, 2H), 2.80 (t, J = 11.8 Hz, 2H), 2.60 (d, J = |
| | | | | 7.1 Hz, 2H), 1.77-1.68 (m, 1H), 1.62 (d, J = 11.1 |
| | | | | Hz, 2H), 1.49-1.37 (m, 2H). |
| 369 | *** | *** | 510.5 | 1H NMR(400 MHz, DMSO-d6) δ 9.01 (s, 1H), 8.56 |
| | | | | (s, 1H), 8.24 (s, 1H), 7.81 (s, 1H), 7.53-7.44 (m, |
| | | | | 1H), 7.26-7.18 (m, 1H), 7.11-7.05 (m, 1H), 5.49 |
| | | | | (s, 1H), 4.67 (s, 1H), 4.53 (s, 1H), 3.96 (s, 3H), 3.60 |
| | | | | (s, 2H), 3.28-3.22 (m, 4H), 2.65-2.56 (m, 4H), |
| | | | | 1.06 (s, 3H), 1.04 (s, 3H). |
| 370 | **** | ** | 510.5 | 1H NMR(400 MHz, DMSO-d6) δ 9.01 (s, 1H), 8.56 |
| | | | | (s, 1H), 8.23 (s, 1H), 7.81 (s, 1H), 7.54-7.44 (m, |
| | | | | 1H), 7.27-7.18 (m, 1H), 7.12-7.04 (m, 1H), 5.48 |
| | | | | (s, 1H), 4.68 (s, 1H), 4.53 (s, 1H), 3.96 (s, 3H), 3.60 |
| | | | | (s, 2H), 3.29-3.24 (m, 4H), 2.65-2.57 (m, 4H), 1.06 |
| | | | | (s, 3H), 1.04 (s, 3H). |
| 371 | *** | ** | 508.4 | 1H NMR(400 MHz, DMSO-d6) δ 9.14 (s, 1H), 8.51 |
| | | | | (s, 1H), 8.16 (s, 1H), 7.77 (s, 1H), 7.64 (s, 1H), 7.36- |
| | | | | 7.24 (m, 2H), 7.06-6.97 (m, 1H), 5.78 (dd, J = 9.0, |
| | | | | 6.0 Hz, 1H), 4.63-4.51 (m, 1H), 3.96 (overlapping s, |
| | | | | 3H), 3.93 (overlapping d, J = 8.9 Hz, 1H), 3.73-3.64 |
| | | | | (m, 2H), 3.61 (dd, J = 8.6, 6.3 Hz, 1H), 3.29-3.25 |
| | | | | (overlapping m, 2H), 2.14-2.00 (m, 2H), 1.90-1.76 |
| | | | | (m, 2H). |
| 372 | *** | ** | 499.5 | 1H NMR(400 MHz, DMSO-d6) δ 7.51-7.40 (m, |
| | | | | 1H), 7.33-7.19 (m, 2H), 7.04-6.89 (m, 2H), 6.36 |
| | | | | (s, 2H), 4.60 (s, 1H), 4.56 (s, 1H), 4.50-4.40 (m, |
| | | | | 1H), 3.78 (dd, J = 11.3, 5.6 Hz, 2H), 3.61-3.52 |
| | | | | (submerged m, 2H), 3.05-2.93 (m, 2H), 2.89 (t, J = |
| | | | | 5.5 Hz, 1H), 2.76 (t, J = 5.6 Hz, 1H), 2.12 (s, 1.7H), |
| | | | | 2.10 (s, 1.3H), 1.96-1.84 (m, 2H), 1.67-1.53 (m, 2H) |
| 373 | *** | ** | 501.8 | 1H NMR(400 MHz, DMSO-d6) δ 7.37-7.24 (m, |
| | | | | 2H), 7.10-6.95 (m, 1H), 4.57 (d, J = 14.1 Hz, 2H), |
| | | | | 4.54-4.48 (m, 1H), 3.76 (t, J = 5.1 Hz, 2H), 3.30- |
| | | | | 3.22 (overlapping m, 2H), 3.26 (overlapping s, 3H), |
| | | | | 3.23-3.13 (m, 1H), 3.02-2.92 (m, 2H), 2.91-2.81 |
| | | | | (m, 2H), 2.73 (t, J = 5.7 Hz, 1H), 2.15-2.02 |
| | | | | (overlapping m, 4H), 2.11 (overlapping s, 1.5H), 2.10 |
| | | | | (overlapping s, 1.5H), , 1.86-1.71 (m, 4H), 1.68- |
| | | | | 1.53 (m, 2H), 1.31-1.16 (m, 2H). |
| 374 | ** | ** | 487.4 | 1H NMR(400 MHz, DMSO-d6) δ 7.36-7.24 (m, |
| | | | | 2H), 7.07-6.97 (m, 1H), 4.63-4.48 (m, 4H), 3.76 (t, |
| | | | | J = 5.4 Hz, 2H), 3.54-3.40 (m, 1H), 3.31-3.18 |
| | | | | (m, 2H), 3.03-2.92 (m, 2H), 2.88-2.77 (m, 2H), 2.75- |
| | | | | 2.69 (m, 1H), 2.10 (s, 1.5H), 2.10 (s, 1.5H), 2.08- |
| | | | | 1.99 (m, 2H), 2.00-1.86 (m, 2H), 1.86-1.74 (m, |
| | | | | 2H), 1.75-1.52 (m, 4H), 1.33-1.20 (m, 2H) |
| 375 | **** | *** | 467.5 | 1H NMR(400 MHz, DMSO-d6) δ 8.27 (s, 1H), 8.04 |
| | | | | (m, 1H), 7.35 (m, 1H), 7.18 (td, J = 10.2, 2.6 Hz, 1H), |
| | | | | 7.03 (td, J = 8.5, 2.3 Hz, 1H), 4.60 (d, J = 15.9 Hz, |
| | | | | 2H), 3.92 (s, 1.28H), 3.91 (s, 1.62H, rotamers), 3.77 |
| | | | | (m, 2H), 3.38 (d, J = 11.0 Hz, 2H), 2.87 (t, J = 5.8 Hz, |
| | | | | 1H), 2.73 (t, J = 5.7 Hz, 1H), 2.60 (m, 4H), 2.11 (s, |
| | | | | 1.62H), 2.10 (s, 1.29H, rotamers), 1.61 (d, J = 10.0 |
| | | | | Hz, 3H), 1.42 (q, J = 12.0 Hz, 2H), |
| 376 | ** | ** | 439.3 | 1H NMR (400 MHz, DMSO-d6) δ 11.47 (br s, 1H), |
| | | | | 8.36 (s, 1H), 8.04 (s, 1H), 7.39-7.24 (overlapping m, |
| | | | | 3H), 7.06-6.97 (m, 1H), 6.39 (d, J = 7.2 Hz, 1H), |
| | | | | 4.62-4.47 (m, 1H), 3.94 (s, 3H), 3.64-3.52 (m, |
| | | | | 2H), 3.23-3.14 (m, 2H), 2.10-1.98 (m, 2H), 1.86- |
| | | | | 1.74 (m, 2H). |
| 377 | *** | ** | 497.5 | 1H NMR(400 MHz, DMSO-d6) δ 9.04 (s, 1H), 8.49 |
| | | | | (s, 1H), 8.15 (s, 1H), 7.49 (s, 1H), 7.36-7.25 (m, |
| | | | | 2H), 7.07-6.97 (m, 1H), 4.70 (d, J = 5.3 Hz, 1H), |
| | | | | 4.64 (t, J = 5.7 Hz, 1H), 4.61-4.53 (m, 1H), 3.98- |
| | | | | 3.89 (m, 4H), 3.69-3.57 (m, 2H), 3.42-3.34 (m, |
| | | | | 2H), 3.28-3.19 (m, 2H), 3.06 (dd, J = 13.7, 4.3 Hz, |
| | | | | 1H), 2.81 (dd, J = 13.7, 8.3 Hz, 1H), 2.13-2.03 (m, |
| | | | | 2H), 1.90-1.78 (m, 2H). |
| 378 | *** | ** | 511.5 | 1H NMR(400 MHz, DMSO-d6) δ 9.04 (s, 1H), 8.50 |
| | | | | (s, 1H), 8.16 (s, 1H), 7.70 (s, 1H), 7.37-7.24 (m, |
| | | | | 2H), 7.07-6.96 (m, 1H), 5.49 (d, J = 5.4 Hz, 1H), |
| | | | | 4.77 (s, 1H), 4.63-4.54 (m, 1H), 4.51 (d, J = 5.4 Hz, |
| | | | | 1H), 3.96 (s, 3H), 3.70-3.58 (m, 2H), 3.32-3.20 |
| | | | | (m, 2H), 2.15-2.03 (m, 2H), 1.90-1.77 (m, 2H), |
| | | | | 1.08 (s, 3H), 1.05 (s, 3H). |
| 379 | *** | ** | 511.5 | 1H NMR(400 MHz, DMSO-d6) δ 9.04 (s, 1H), 8.50 |
| | | | | (s, 1H), 8.16 (s, 1H), 7.70 (s, 1H), 7.37-7.23 (m, |
| | | | | 2H), 7.08-6.93 (m, 1H), 5.49 (d, J = 5.4 Hz, 1H), |
| | | | | 4.77 (s, 1H), 4.62-4.54 (m, 1H), 4.51 (d, J = 5.4 Hz, |
| | | | | 1H), 3.96 (s, 3H), 3.70-3.59 (m, 2H), 3.29-3.21 |
| | | | | (m, 2H), 2.13-2.03 (m, 2H), 1.90-1.78 (m, 2H), |
| | | | | 1.08 (s, 3H), 1.05 (s, 3H). |
| 380 | *** | ** | 496.4 | 1H NMR(400 MHz, DMSO-d6) δ 8.66 (s, 1H), 8.34 |
| | | | | (s, 1H), 8.03 (s, 1H), 7.34-7.25 (m, 2H), 7.06-6.94 |
| | | | | (m, 1H), 6.78 (t, J = 5.5 Hz, 1H), 6.48 (s, 1H), 4.64- |
| | | | | 4.45 (m, 1H), 3.93 (s, 3H), 3.60-3.43 (m, 6H), 3.29 |
| | | | | (s, 3H), 3.21-3.07 (m, 2H), 2.10-1.98 (m, 2H), |
| | | | | 1.87-1.73 (m, 2H) |
| 381 | *** | ** | 497.4 | 1H NMR(400 MHz, DMSO-d6) δ 8.85-8.78 (m, |
| | | | | 1H), 8.43 (s, 1H), 8.13-8.07 (m, 1H), 7.37-7.22 |
| | | | | (m, 2H), 7.07-6.95 (m, 1H), 6.92-6.87 (m, 1H), |
| | | | | 4.65-4.52 (m, 1H), 4.51-4.41 (m, 2H), 3.94 (s, |
| | | | | 3H), 3.72-3.67 (m, 2H), 3.67-3.58 (m, 2H), 3.33 |
| | | | | (s, 3H), 3.28-3.18 (m, 2H), 2.11-2.01 (m, 2H), |
| | | | | 1.90-1.74 (m, 2H). |
| 382 | *** | ** | 495.4 | 1H NMR(400 MHz, DMSO-d6) δ 8.85-8.73 (m, |
| | | | | 1H), 8.43 (s, 1H), 8.14-8.02 (m, 1H), 7.37-7.23 |
| | | | | (m, 2H), 7.06-6.96 (m, 1H), 6.96-6.89 (m, 1H), |
| | | | | 5.71-5.59 (m, 1H), 4.94 (t, J = 7.1 Hz, 2H), 4.69- |
| | | | | 4.59 (m, 2H), 4.59-4.51 (m, 1H), 3.94 (s, 3H), 3.72- |
| | | | | 3.58 (m, 2H), 3.31-3.18 (m, 2H), 2.12-2.01 (m, |
| | | | | 2H), 1.93-1.75 (m, 2H). |
| 383 | ** | ** | 437.2 | 1H NMR(400 MHz, DMSO-d6) δ 8.03 (d, J = 5.7 Hz, |
| | | | | 1H), 7.75 (s, 1H), 7.30 (dd, J = 15.5, 8.2 Hz, 1H), |
| | | | | 6.79 (ddt, J = 17.3, 8.9, 2.3 Hz, 3H), 5.07 (s, 1H), |
| | | | | 4.62-4.54 (m, 2H), 3.87 (s, 3H), 3.84-3.69 (m, |
| | | | | 2H), 3.66-3.58 (m, 1H), 3.52-3.42 (m, 1H), 3.23 |
| | | | | (d, J = 11.8 Hz, 2H), 2.93-2.70 (m, 2H), 2.29-2.18 |
| | | | | (m, 1H), 2.11 (d, J = 8.7 Hz, 3H), 2.07 (d, J = 6.0 Hz, 1H). |
| 384 | ** | ** | 500.3 | 1H NMR(400 MHz, DMSO-d6) δ 8.27 (s, 1H), 8.05 |
| | | | | (s, 1H), 7.70 (d, J = 8.1 Hz, 2H), 7.57 (d, J = 8.1 Hz, |
| | | | | 2H), 4.61 (d, J = 15.6 Hz, 2H), 3.90 (d, J = 1.6 Hz, |
| | | | | 3H), 3.78 (d, J = 4.5 Hz, 2H), 3.66 (s, 2H), 3.07 (s, |
| | | | | 4H), 2.89 (t, J = 5.7 Hz, 1H), 2.76 (t, J = 5.6 Hz, 1H), |
| | | | | 2.57 (s, 4H), 2.12 (d, J = 6.0 Hz, 3H). |
| 385 | * | ** | 478.3 | 1H NMR(400 MHz, DMSO-d6) δ 7.99 (d, J = 5.2 Hz, |
| | | | | 1H), 7.69 (s, 1H), 7.44-7.37 (m, 2H), 7.28 (t, J = 8.5 |
| | | | | Hz, 2H), 4.55 (d, J = 17.1 Hz, 2H), 3.87 (s, 3H), 3.82- |
| | | | | 3.68 (m, 2H), 3.42(S, 1H), 3.14 (d, J = 11.4 Hz, 6H), |
| | | | | 2.85 (dd, J = 13.2, 6.1 Hz, 2H), 2.73-2.65 (m, 1H), |
| | | | | 2.13-2.07 (m, 3H), 2.02-1.90 (m, 3H), 1.81 (s, 2H). |
| 386 | * | ** | 496.3 | 1H NMR(400 MHz, DMSO-d6) δ 8.46 (t, J = 5.6 Hz, |
| | | | | 1H), 8.03 (d, J = 5.8 Hz, 1H), 7.72 (s, 1H), 7.31 (dd, |
| | | | | J = 15.4, 8.6 Hz, 1H), 7.24-7.17 (m, 1H), 7.09-7.00 |
| | | | | (m, 1H), 4.57 (d, J = 16.2 Hz, 2H), 4.26 (d, J = 5.6 |
| | | | | Hz, 2H), 3.88 (s, 3H), 3.83-3.70 (m, 2H), 3.36 (d, |
| | | | | J = 11.8 Hz, 1H), 3.25 (dd, J = 9.6, 5.4 Hz, 3H), 3.02- |
| | | | | 2.92 (m, 1H), 2.85 (d, J = 5.5 Hz, 1H), 2.72 (s, 1H), |
| | | | | 2.11 (d, J = 8.1 Hz, 3H), 2.08-2.00 (m, 1H), 2.00- |
| | | | | 1.90 (m, 1H). |
| 387 | ** | ** | 510.3 | 1H NMR(400 MHz, DMSO-d6) δ 8.04 (dd, J = 9.4, |
| | | | | 5.8 Hz, 1H), 7.73 (d, J = 8.7 Hz, 1H), 7.31-7.19 (m, |
| | | | | 2H), 7.12-7.01 (m, 1H), 4.69-4.45 (m, 4H), 3.88 |
| | | | | (s, 3H), 3.78 (dd, J = 12.6, 6.8 Hz, 2H), 3.51-3.39 |
| | | | | (m, 3H), 3.24 (d, J = 7.1 Hz, 2H), 3.00 (s, 2H), 2.86 |
| | | | | (s, 1H), 2.75 (d, J = 14.0 Hz, 2H), 2.19-1.88 (m, 5H). |
| 388 | * | *** | 398.3 | 1H NMR(400 MHz, DMSO-d6) δ 8.04 (d, J = 5.8 Hz, |
| | | | | 1H), 7.73 (d, J = 0.6 Hz, 1H), 4.57 (d, J = 17.6 Hz, |
| | | | | 2H), 3.88 (s, 3H), 3.83-3.70 (m, 2H), 3.39-3.34 |
| | | | | (m, 2H), 3.31-3.26 (m, 2H), 3.23-3.15 (m, 1H), |
| | | | | 3.01 (s, 3H), 2.88-2.80 (m, 4H), 2.72 (s, 1H), 2.13- |
| | | | | 2.02 (m, 4H), 1.97-1.87 (m, 1H). |
| 389 | * | ** | 440.3 | 1H NMR(400 MHz, DMSO-d6) δ 8.04 (d, J = 6.1 Hz, |
| | | | | 1H), 7.73 (s, 1H), 4.57 (d, J = 16.7 Hz, 2H), 3.89 (s, |
| | | | | 3H), 3.82-3.71 (m, 2H), 3.53 (d, J = 5.6 Hz, 9H), |
| | | | | 3.47-3.35 (m, 2H), 3.24-3.15 (m, 1H), 2.86 (d, J = |
| | | | | 3.1 Hz, 1H), 2.73 (s, 1H), 2.11 (d, J = 8.0 Hz, 3H), |
| | | | | 2.08-2.01 (m, 1H), 2.00-1.87 (m, 1H). |
| 390 | ** | ** | 467.3 | 1H NMR(400 MHz, DMSO-d6) δ 8.04 (d, J = 5.7 Hz, |
| | | | | 1H), 7.73 (s, 1H), 4.57 (d, J = 16.6 Hz, 2H), 4.15 (s, |
| | | | | 1H), 3.99 (s, 1H), 3.89 (s, 3H), 3.78 (p, J = 14.0 Hz, |
| | | | | 4H), 3.46-3.35 (m, 3H), 3.28 (s, 2H), 3.21 (d, J = |
| | | | | 4.9 Hz, 1H), 2.85 (s, 4H), 2.73 (s, 1H), 2.11 (d, J = |
| | | | | 7.9 Hz, 4H), 1.94 (dd, J = 18.9, 7.2 Hz, 1H). |
| 391 | * | ** | 464.3 | 1H NMR(400 MHz, DMSO-d6) δ 7.85 (d, J = 14.8 |
| | | | | Hz, 2H), 7.48-7.45 (m, 2H), 7.03 (t, J = 8.4 Hz, 2H), |
| | | | | 4.58 (d, J = 16.5 Hz, 2H), 3.88 (s, 4H), 3.80-3.56 |
| | | | | (m, 3H), 3.43 (m, 2H), 3.13 (m, 1H), 2.86 (d, J = 4.4 |
| | | | | Hz, 2H), 2.19-2.02 (m, 5H). |
| 392 | * | ** | 496.3 | 1H NMR(400 MHz, DMSO-d6) δ 8.02-7.97 (m, |
| | | | | 1H), 7.70 (d, J = 7.1 Hz, 1H), 7.67-7.53 (m, 1H), |
| | | | | 7.46 (dt, J = 18.2, 7.8 Hz, 1H), 7.20 (dd, J = 17.8, 9.2 |
| | | | | Hz, 1H), 4.63-4.50 (m, 2H), 3.87 (s, 3H), 3.84- |
| | | | | 3.67 (m, 3H), 3.32 (s, 25H), 3.23-3.14 (m, 2H), 3.08 |
| | | | | (d, J = 10.5 Hz, 4H), 2.83 (dt, J = 15.0, 7.5 Hz, 2H), |
| | | | | 2.77-2.64 (m, 1H), 2.48-2.36 (m, 1H), 2.10 (d, J = |
| | | | | 8.5 Hz, 3H), 2.05-1.72 (m, 2H). |
| 393 | ** | ** | 451.4 | 1H NMR(400 MHz, DMSO-d6) δ 8.32 (s, 1H), 8.05 |
| | | | | (d, J = 1.8 Hz, 1H), 7.31 (dd, J = 15.5, 8.2 Hz, 1H), |
| | | | | 6.86 (ddd, J = 15.3, 7.3, 2.0 Hz, 2H), 6.75 (td, J = 8.4, |
| | | | | 2.0 Hz, 1H), 4.62 (d, J = 16.2 Hz, 3H), 3.92 (s, 3H), |
| | | | | 3.84-3.75 (m, 2H), 3.04-2.94 (m, 2H), 2.90 (t, J = |
| | | | | 5.6 Hz, 2H), 2.81-2.72 (m, 1H), 2.67- |
| | | | | 2.66(m, 1H), 2.04 (m, 5H), 1.88-1.76 (m, 2H). |
| 394 | *** | ** | 483.4 | 1H NMR(400 MHz, DMSO-d6) δ 8.33 (s, 1H), 8.06 |
| | | | | (s, 1H), 7.35-7.24 (m, 2H), 7.06-6.96 (m, 1H), |
| | | | | 5.18-5.05 (m, 1H), 4.74 (d, J = 17.2 Hz, 0.5H), 4.56- |
| | | | | 4.45 (m, 2H), 4.03 (d, J = 18.3 Hz, 0.5H), 3.92 (s, |
| | | | | 3H), 3.32-3.14 (overlapping m, 3H), 3.04-2.90 (m, |
| | | | | 2H), 2.69-2.57 (m, 1H), 2.14 (s, 1.6H), 2.11 (s, |
| | | | | 1.4H), 2.09-2.01 (m, 2H), 1.90-1.77 (m, 2H), 1.16 |
| | | | | (d, J = 6.8 Hz, 1.6 H), 1.03 (d, J = 6.9 Hz, 1.4H). |
| 395 | ** | ** | 439.4 | 1H NMR (400 MHz, DMSO-d6) δ 11.46-11.13 (br. |
| | | | | s, 1H), 8.55 (s, 1H), 8.36 (s, 1H), 8.04 (s, 1H), 7.36- |
| | | | | 7.24 (m, 2H), 7.06-6.97 (m, 1H), 6.52 (s, 1H), 4.61- |
| | | | | 4.48 (m, 1H), 3.93 (s, 3H), 3.66-3.54 (m, 2H), |
| | | | | 3.25-3.12 (m, 2H, overlapping), 2.11-1.98 (m, |
| | | | | 2H), 1.88-1.72 (m, 2H). |
| 396 | *** | ** | 546.4 | 1H NMR(400 MHz, DMSO-d6) δ 9.09 (d, J = 0.6 Hz, |
| | | | | 1H), 8.90 (s, 1H), 8.43 (s, 1H), 7.94 (t, J = 58.7 Hz, |
| | | | | 1H), 7.72 (s, 1H), 7.51-7.43 (m, 1H), 7.21 (ddd, J = |
| | | | | 10.2, 9.6, 2.6 Hz, 1H), 7.07 (td, J = 8.5, 2.1 Hz, 1H), |
| | | | | 5.51 (d, J = 5.2 Hz, 1H), 4.74 (s, 1H), 4.52 (d, J = 5.1 |
| | | | | Hz, 1H), 3.57 (s, 2H), 3.43-3.25 (overlapped m, |
| | | | | 4H), 2.59-2.53 (overlapped m, 4H), 1.07 (s, 3H), |
| | | | | 1.06 (s, 3H). |
| 397 | **** | ** | 546.4 | 1H NMR(400 MHz, DMSO-d6) δ 9.09 (d, J = 0.6 Hz, |
| | | | | 1H), 8.90 (s, 1H), 8.43 (s, 1H), 7.94 (t, J = 58.8 Hz, |
| | | | | 1H), 7.72 (s, 1H), 7.52-7.43 (m, 1H), 7.26-7.18 |
| | | | | (m, 1H), 7.07 (td, J = 8.4, 2.0 Hz, 1H), 5.51 (d, J = 5.3 |
| | | | | Hz, 1H), 4.74 (s, 1H), 4.52 (d, J = 5.0 Hz, 1H), 3.57 |
| | | | | (s, 2H), 3.41-3.32 (overlapped m, 4H), 2.61-2.53 |
| | | | | (overlapped m, 4H), 1.07 (s, 3H), 1.06 (s, 3H). |
| 398 | ** | ** | 441.4 | 1H NMR(400 MHz, DMSO-d6) δ 1H NMR (400 |
| | | | | MHz, DMSO-d6) NMR 8.07 (d, J = 3.1 Hz, 1H), |
| | | | | 7.77 (s, 1H), 7.36-7.29 (m, 1H), 7.01-6.96 (m, |
| | | | | 2H), 5.09-5.02 (m, 1H), 4.61 (s, 0.88 H), 4.57 (s, |
| | | | | 1.11H, rotamers), 4.29 (t, J = 8.1 Hz, 2H), 3.90 (s, |
| | | | | 1.3H), 3.90 (s, 1.7H, rotamers), 3.84-3.75 (m, 4H), |
| | | | | 2.89 (t, J = 5.9 Hz, 1H), 2.76 (t, J = 5.9 Hz, 1H), 2.12 |
| | | | | (s, 1.67H), 2.10 (s, 1.3H) |
| 399 | ** | ** | 483.4 | 1H NMR(400 MHz, DMSO-d6) δ 8.84-8.78 (m, |
| | | | | 1H), 8.43 (s, 1H), 8.12-8.08 (m, 1H), 7.36-7.21 |
| | | | | (m, 2H), 7.08-6.94 (m, 1H), 6.92-6.84 (m, 1H), |
| | | | | 4.89 (t, J = 5.5 Hz, 1H), 4.62-4.51 (m, 1H), 4.39- |
| | | | | 4.30 (m, 2H), 3.94 (s, 3H), 3.75 (dd, J = 10.2, 5.2 Hz, |
| | | | | 2H), 3.69-3.57 (m, 2H), 3.28-3.17 (m, 2H), 2.11- |
| | | | | 1.99 (m, 2H), 1.89-1.75 (m, 2H) |
| 400 | *** | ** | 515.5 | 1H NMR(400 MHz, DMSO-d6) δ 8.36 (s, 1H), 8.06 |
| | | | | (d, J = 2.0 Hz, 1H), 7.46 (dd, J = 8.5, 0.9 Hz, 1H), |
| | | | | 7.39 (d, J = 1.9 Hz, 1H), 7.05-6.98 (m, 1H), 4.86- |
| | | | | 4.73 (m, 1H), 4.64 (s, 1H), 4.60 (s, 1H), 4.22 (q, J = |
| | | | | 7.2 Hz, 2H), 3.85-3.71 (m, 2H), 3.32-3.24 |
| | | | | (overlapping m, 2H), 3.09-2.98 (m, 2H), 2.90 (t, J = |
| | | | | 5.6 Hz, 1H), 2.76 (t, J = 5.6 Hz, 1H), 2.12 (s, 1.6H), |
| | | | | 2.11 (s, 1.4H), 2.10-2.04 (overlapping m, 2H), 1.92- |
| | | | | 1.79 (m, 2H), 1.41 (t, J = 7.2 Hz, 3H) |
| 401 | *** | ** | 497.4 | 1H NMR(400 MHz, DMSO-d6) δ 9.06 (s, 0.2 H), |
| | | | | 9.05 (s, 0.8 H), 9.65 (s, 1H), 8.50 (s, 1H), 8.16 (, |
| | | | | 1Hs), 7.82 (s, 0.1H), 7.65 (s, 0.9H), 7.37-7.24 (m, |
| | | | | 2H), 7.06-6.97 (m, 1H), 5.36 (d, J = 5.8 Hz, 0.85H), |
| | | | | 5.20 (s, 0.12H), 4.63-4.53 (m, 1H), 4.53-4.45 (m, |
| | | | | 2H), 4.00-3.96 (overlapping m, 1H), 3.95 |
| | | | | (overlapping s, 3H), 3.71-3.57 (m, 2H), 3.30-3.18 |
| | | | | (m, 2H), 2.14-2.01 (m, 2H), 1.92-1.77 (m, 2H), |
| | | | | 1.45 (s, 0.4H), 1.06 (d, 2.7H). |
| 402 | ** | ** | 492.3 | 1H NMR(400 MHz, DMSO-d6) δ 9.08 (s, 1H), 8.50 |
| | | | | (s, 1H), 8.16 (s, 1H), 7.74 (s, 1H), 7.37-7.25 (m, |
| | | | | 2H), 7.02 (t, J = 8.1 Hz, 1H), 6.32 (bs, 1H), 5.14- |
| | | | | 4.98 (m, 1H), 4.66-4.46 (m, 1H), 3.96 (s, 3H), 3.74- |
| | | | | 3.59 (m, 2H), 3.28-3.22 (overlapping m, 2H), 3.17- |
| | | | | 2.97 (m, 2H), 2.14-2.02 (m, 2H), 1.91-1.78 (m, 2H). |
| 403 | *** | ** | 531.4 | 1H NMR(400 MHz, DMSO-d6) δ 8.36 (s, 1H), 8.10- |
| | | | | 7.97 (m, 1H), 7.47 (s, 0.5H), 7.45 (s, 0.5H), 7.39 (d, |
| | | | | J = 2.3 Hz, 1H), 7.04 (d, J = 2.3 Hz, 0.5H), 7.01 (d, J = |
| | | | | 2.3 Hz, 0.5H), 4.84-4.74 (m, 1H), 4.75-4.67 (m, |
| | | | | 1H), 4.63 (s, 1H), 4.57 (s, 1H), 4.30-4.15 (m, 4H), |
| | | | | 3.84 (t, J = 5.5 Hz, 1H), 3.72 (t, J = 5.6 Hz, 1H), 3.36- |
| | | | | 3.24 (overlapping m, 2H), 3.16-2.99 (m, , 2H), |
| | | | | 2.89 (t, J = 5.2 Hz, 1H), 2.80 (t, J = 5.0 Hz, 1H), 2.18- |
| | | | | 2.01 (m, 2H), 1.96-1.78 (m, 2H), 1.41 (t, J = 7.3 |
| | | | | Hz, 3H |
| 404 | ** | ** | 485.4 | 1H NMR(400 MHz, DMSO-d6) δ 7.38-7.25 (m, |
| | | | | 2H), 7.10-6.95 (m, 1H), 4.74-4.43 (m, 3H), 3.77 (t, |
| | | | | J = 5.3 Hz, 2H), 3.53-3.39 (m, 1H), 3.36-3.33 |
| | | | | (overlapping m, 1H), 3.31-3.28 (overlapping m, |
| | | | | 1H), 3.07-2.96 (m, 2H), 2.88 (t, J = 5.8 Hz, 1H), |
| | | | | 2.75 (t, J = 5.8 Hz, 1H), 2.68-2.56 (m, 2H), 2.37- |
| | | | | 2.23 (m, 2H), 2.15-2.11 (overlapping m, 1H), 2.11 |
| | | | | (overlapping s, 1.5H), 2.09 (overlapping s, 1.5H), |
| | | | | 2.06-1.99 (overlapping m, 3H), 1.97-1.80 |
| | | | | (overlapping m, 4H) |
| 405 | *** | ** | 472.5 | 1H NMR(400 MHz, DMSO-d6) δ 8.32 (s, 1H), 8.05 |
| | | | | (d, J = 1.8 Hz, 1H), 7.34-7.24 (m, 2H), 7.04-6.97 |
| | | | | (m, 1H), 4.64 (s, 0.84H), 4.60 (s, 1.14H, rotamers), |
| | | | | 4.55-4.46 (m, 1H), 3.79 (dd, J = 9.8, 5.7 Hz, 2H), |
| | | | | 3.31-3.27 (overlapped, m, 2H), 2.96 (t, J = 9.7 Hz, |
| | | | | 2H), 2.90 (t, J = 5.8 Hz, 1H), 2.76 (t, J = 5.8 Hz, 1H), |
| | | | | 2.12 (s, 1.59H), 2.11 (s, 1.37H, rotamers), 2.09-2.03 |
| | | | | (m, 2H), 1.90-1.77 (m, 2H) |
| 406 | **** | ** | 488.5 | 1H NMR(400 MHz, DMSO-d6) δ 8.36 (s, 1H), 8.06- |
| | | | | 8.05 (m, 1H), 7.34-7.24 (m, 2H), 7.05-6.97 (m, |
| | | | | 1H), 4.64 (s, 0.85H), 4.60 (s, 1.11H, rotamers), 4.55- |
| | | | | 4.46 (m, 1H), 3.79 (dd, J = 9.8, 5.8 Hz, 2H), 3.31- |
| | | | | 3.28 (overlapped, m, 2H), 2.96 (t, J = 9.9 Hz, 2H), |
| | | | | 2.90 (t, J = 5.8 Hz, 1H), 2.76 (t, J = 5.8 Hz, 1H), 2.12 |
| | | | | (s, 1.62H), 2.11 (s, 1.30H, rotamers), 2.09-2.02 (m, |
| | | | | 2H), 1.89-1.78 (m, 2H) |
| 407 | *** | ** | 547.4 | 1H NMR(400 MHz, DMSO-d6) δ 9.13-9.07 (s, 1H), |
| | | | | 8.96 (s, 1H), 8.47 (s, 1H), 7.96 (t, J = 58.6 Hz, 1H), |
| | | | | 7.73 (s, 1H), 7.37-7.26 (m, 2H), 7.04-6.98 (m, |
| | | | | 1H), 5.51 (d, J = 5.4 Hz, 1H), 4.75 (s, 1H), 4.62- |
| | | | | 4.55 (m, 1H), 4.53 (d, J = 5.3 Hz, 1H), 3.70-3.57 |
| | | | | (m, 2H), 3.30-3.20 (overlapped m, 2H), 2.13-2.01 |
| | | | | (m, 2H), 1.91-1.76 (m, 2H), 1.08 (s, 3H), 1.06 (s, 3H). |
| 408 | *** | ** | 547.4 | 1H NMR(400 MHz, DMSO-d6) δ 8.33 (s, 1H), 8.04 |
| | | | | (s, 1H), 7.47 (s, 0.5H), 7.45 (s, 0.5H), 7.39 (d, J = 2.3 |
| | | | | Hz, 1H), 7.03 (d, J = 2.3 Hz, 0.5H), 7.01 (d, J = 2.3 |
| | | | | Hz, 0.5H), 4.82-4.71 (m, 1H), 4.58-4.51 (m, 2H), |
| | | | | 4.21 (q, J = 7.2 Hz, 2H), 3.76-3.67 (m, 1H), 3.67- |
| | | | | 3.61 (m, 2H), 3.41-3.34 (overlapping m, 2H), 3.32- |
| | | | | 3.22 (m, 2H), 3.08-2.96 (m, 2H), 2.87-2.84 |
| | | | | (overlapping m, 1H), 2.82 (s, 3H), 2.62 (dd, J = 12.7, |
| | | | | 4.7 Hz, 1H), 2.48-2.41 (overlapping m, 1H), 2.19- |
| | | | | 1.99 (m, 2H), 1.93-1.74 (m, 2H), 1.40 (t, J = 7.3 Hz, 3H) |
| 409 | *** | *** | 448.0 | 1H NMR(400 MHz, DMSO-d6) δ 8.64 (d, J = 5.6 Hz, |
| | | | | 1H), 8.55 (s, 1H), 8.18 (s, 1H), 7.88 (d, J = 5.6 Hz, |
| | | | | 1H), 7.35-7.26 (m, 2H), 7.05-6.98 (m, 1H), 4.62- |
| | | | | 4.54 (m, 1H), 3.98 (s, 3H), 3.80-3.69 (m, 2H), 3.32- |
| | | | | 3.24 (overlapping m, 2H), 2.12-2.00 (m, 2H), 1.89- |
| | | | | 1.72 (m, 2H). |
| 410 | ** | ** | 453.1 | 1H NMR(400 MHz, DMSO-d6) δ 8.83 (s, 1H), 8.43 |
| | | | | (s, 1H), 8.10 (s, 1H), 7.39-7.22 (m, 2H), 7.08-6.97 |
| | | | | (m, 1H), 6.92 (s, 1H), 4.62-4.52 (m, 1H), 4.01- |
| | | | | 3.90 (m, 6H), 3.68-3.57 (m, 2H), 3.29-3.18 |
| | | | | (overlapping t, J = 10.4 Hz, 2H), 2.12-2.02 (m, 2H), |
| | | | | 1.88-1.77 (m, 2H). |
| 411 | ** | ** | 457.3 | 1H NMR (400 MHz, DMSO-d6) δ 9.03 (s, 1H), 8.67 |
| | | | | (s, 1H), 8.51 (s, 1H), 8.15 (s, 1H), 7.37-7.25 (m, |
| | | | | 2H), 7.09-6.96 (m, 1H), 4.67-4.53 (m, 1H), 3.96 |
| | | | | (s, 3H), 3.80-3.69 (m, 2H), 3.42-3.34 (m, 2H), |
| | | | | 2.15-2.01 (m, 2H), 1.92-1.77 (m, 2H). |
| 412 | ** | ** | 525.4 | 1H NMR(400 MHz, DMSO-d6) δ 9.10 (d, J = 0.6 Hz, |
| | | | | 1H), 8.51 (s, 1H), 8.16 (d, J = 0.4 Hz, 1H), 7.91 (d, |
| | | | | J = 0.6 Hz, 1H), 7.37-7.25 (m, 2H), 7.06-6.98 (m, |
| | | | | 1H), 5.36 (s, 1H), 5.26 (d, J = 6.6 Hz, 1H), 4.65- |
| | | | | 4.53 (m, 2H), 4.24 (d, J = 9.5 Hz, 1H), 4.03 (t, J = 7.5 |
| | | | | Hz, 1H), 3.95 (s, 3H), 3.87 (d, J = 9.1 Hz, 1H), 3.71- |
| | | | | 3.61 (m, 3H), 3.31-3.21 (m, 2H), 2.13-2.01 (m, |
| | | | | 2H), 1.91-1.76 (m, 2H). |
| 413 | *** | ** | 509.4 | 1H NMR(400 MHz, DMSO-d6) δ 9.15 (d, J = 0.6 Hz, |
| | | | | 1H), 8.56 (s, 1H), 8.19 (d, J = 0.6 Hz, 1H), 8.16 (d, |
| | | | | J = 0.5 Hz, 1H), 7.37-7.24 (m, 2H), 7.07-6.96 (m, |
| | | | | 1H), 5.96 (s, 1H), 4.65-4.54 (m, 1H), 3.96 (s, 3H), |
| | | | | 3.78-3.68 (m, 2H), 3.41-3.29 (overlapped m, 2H), |
| | | | | 2.13-2.02 (m, 2H), 1.91-1.78 (m, 2H), 1.55 (s, 6H). |
| 414 | *** | ** | 496.4 | 1H NMR(400 MHz, DMSO-d6) δ 9.06 (s, 1H), 8.50 |
| | | | | (s, 1H), 8.37 (bs, formate 1H), 8.15 (s, 1H), 7.71 (s, |
| | | | | 1H), 7.39-7.22 (m, 2H), 7.08-6.95 (m, 1H), 4.98 |
| | | | | (d, J = 6.2 Hz, 1H), 4.67-4.47 (m, 1H), 3.95 (s, 3H), |
| | | | | 3.74-3.56 (m, 2H), 3.35-3.13 (m, 3H), 2.94 (t, J = |
| | | | | 5.8 Hz, 1H), 2.49 (overlapping s, 3H), 2.16-1.99 |
| | | | | (m, 2H), 1.91-1.72 (m, 2H). |
| 415 | *** | ** | 497.5 | 1H NMR(400 MHz, DMSO-d6) δ 9.04 (s, 1H), 8.49 |
| | | | | (s, 1H), 8.15 (s, 1H), 7.49 (s, 1H), 7.36-7.25 (m, |
| | | | | 2H), 7.06-6.98 (m, 1H), 4.69 (d, J = 5.3 Hz, 1H), |
| | | | | 4.63 (t, J = 5.7 Hz, 1H), 4.60-4.53 (m, 1H), 3.98- |
| | | | | 3.89 (m, 4H), 3.68-3.58 (m, 2H), 3.43-3.34 (m, |
| | | | | 2H), 3.28-3.17 (m, 2H), 3.06 (dd, J = 13.7, 4.4 Hz, |
| | | | | 1H), 2.81 (dd, J = 13.7, 8.3 Hz, 1H), 2.15-2.01 (m, |
| | | | | 2H), 1.89-1.75 (m, 2H). |
| 416 | *** | ** | 497.5 | 1H NMR(400 MHz, DMSO-d6) δ 9.00 (s, 1H), 8.45 |
| | | | | (s, 1H), 8.11 (s, 1H), 7.45 (s, 1H), 7.32-7.20 (m, |
| | | | | 2H), 7.03-6.92 (m, 1H), 4.65 (d, J = 5.3 Hz, 1H), |
| | | | | 4.59 (t, J = 5.7 Hz, 1H), 4.55-4.49 (m, 1H), 3.96- |
| | | | | 3.84 (m, 4H), 3.66-3.54 (m, 2H), 3.40-3.30 (m, |
| | | | | 2H), 3.25-3.14 (m, 2H), 3.02 (dd, J = 13.7, 4.4 Hz, |
| | | | | 1H), 2.78 (dd, J = 13.8, 8.3 Hz, 1H), 2.09-1.97 (m, |
| | | | | 2H), 1.87-1.73 (m, 2H). |
| 417 | *** | ** | 436.2 | 1HNMR (400 MHz, DMSO-d6) δ 9.48 (dd, J = 3.6, |
| | | | | 1.2 Hz, 1H), 8.57 (s, 1H), 8.20 (s, 1H), 7.18 (t, J = 9.4 |
| | | | | Hz, 1H), 6.89 (dd, J = 13.0, 3.0 Hz, 1H), 6.73-6.68 |
| | | | | (m, 1H), 4.49-4.41 (m, 1H), 3.97 (s, 2H), 3.72 (s, |
| | | | | 3H), 3.35 (d, J = 12.6 Hz, 2H), 2.05 (dd, J = 12.6, 3.6 |
| | | | | Hz, 1H), 1.87-1.77 (m, 1H). |
| 418 | ** | ** | 419.2 | 1H NMR(400 MHz, DMSO-d6) δ 8.03 (d, J = 5.5 Hz, |
| | | | | 1H), 7.74 (s, 1H), 7.29-7.25 (m, 2H), 6.94-6.90 |
| | | | | (m, 2H), 5.00 (s, 1H), 4.62-4.54 (m, 2H), 3.87 (s, |
| | | | | 3H), 3.85-3.67 (m, 2H), 3.63-3.56 (m, 1H), 3.53- |
| | | | | 3.43 (m, 1H), 2.92-2.82 (m, 1H), 2.76-2.70 (m, |
| | | | | 2H), 2.25-2.15 (m, 1H), 2.11 (d, J = 8.7 Hz, 3H), |
| | | | | 2.06 (d, J = 11.2 Hz, 1H). |
| 419 | ** | ** | 433.3 | 1H NMR(400 MHz, DMSO-d6) δ 8.32 (s, 1H), 8.05 |
| | | | | (d, J = 1.9 Hz, 1H), 7.29 (dd, J = 8.6, 7.4 Hz, 2H), |
| | | | | 6.98 (d, J = 7.8 Hz, 2H), 6.92 (t, J = 7.3 Hz, 1H), 4.67- |
| | | | | 4.53 (m, 3H), 3.91 (t, J = 5.1 Hz, 3H), 3.83-3.75 |
| | | | | (m, 2H), 3.35 (s, 2H), 3.04-2.94 (m, 2H), 2.90 (t, J = |
| | | | | 5.7 Hz, 1H), 2.77 (t, J = 5.7 Hz, 1H), 2.15-2.04 (m, |
| | | | | 5H), 1.88-1.76 (m, 2H). |
| 420 | *** | ** | 451.3 | 1H NMR(400 MHz, DMSO-d6) δ 8.35 (s, 1H), 8.08 |
| | | | | (d, J = 2.0 Hz, 1H), 7.26 (tdd, J = 11.7, 8.3, 1.4 Hz, |
| | | | | 2H), 7.14 (t, J = 7.8 Hz, 1H), 7.01-6.93 (m, 1H), |
| | | | | 4.69-4.56 (m, 3H), 3.94 (d, J = 1.8 Hz, 3H), 3.86- |
| | | | | 3.77 (m, 2H), 3.34-3.27 (m, 2H), 3.00 (t, J = 9.8 Hz, |
| | | | | 2H), 2.93 (t, J = 5.7 Hz, 1H), 2.79 (t, J = 5.6 Hz, 1H), |
| | | | | 2.18-2.07 (m, 5H), 1.93-1.83 (m, 2H). |
| 421 | ** | ** | 451.4 | 1H NMR(400 MHz, DMSO-d6) δ 8.32 (s, 1H), 8.05 |
| | | | | (d, J = 1.8 Hz, 1H), 7.16-7.07 (m, 2H), 7.04-6.97 |
| | | | | (m, 2H), 4.62 (d, J = 16.1 Hz, 2H), 4.54-4.46 (m, |
| | | | | 1H), 3.92 (d, J = 1.6 Hz, 3H), 3.82-3.76 (m, 2H), |
| | | | | 3.32-3.25 (m, 2H), 2.93 (dt, J = 11.4, 8.3 Hz, 3H), |
| | | | | 2.77 (t, J = 5.6 Hz, 1H), 2.09 (dd, J = 21.7, 8.3 Hz, |
| | | | | 5H), 1.86-1.75 (m, 2H). |
| 422 | * | ** | 339.2 | 1H NMR(400 MHz, DMSO-d6) δ 8.21 (d, J = 1.9 Hz, |
| | | | | 1H), 7.97 (s, 1H), 5.87 (d, J = 10.1 Hz, 1H), 5.79 (d, |
| | | | | J = 10.1 Hz, 1H), 4.61 (d, J = 15.6 Hz, 2H), 3.89 (s, |
| | | | | 3H), 3.78 (dd, J = 10.0, 5.6 Hz, 2H), 3.57 (d, J = 2.1 |
| | | | | Hz, 2H), 3.25-3.17 (m, 2H), 2.89 (t, J = 5.7 Hz, 1H), |
| | | | | 2.76 (t, J = 5.7 Hz, 1H), 2.23 (s, 2H), 2.12 (d, J = 5.5 |
| | | | | Hz, 3H). |
| 423 | ** | ** | 469.3 | 1H NMR(400 MHz, DMSO-d6) δ 8.33 (s, 1H), 8.06 |
| | | | | (d, J = 2.0 Hz, 1H), 7.16-7.09 (m, 2H), 7.05-6.95 |
| | | | | (m, 1H), 4.62 (d, J = 16.2 Hz, 3H), 3.92 (s, 3H), 3.84- |
| | | | | 3.73 (m, 2H), 3.34 (s, 2H), 2.99 (t, J = 9.8 Hz, 2H), |
| | | | | 2.90 (t, J = 5.5 Hz, 1H), 2.77 (t, J = 5.7 Hz, 1H), 2.12 |
| | | | | (d, J = 4.8 Hz, 5H), 1.88 (dd, J = 10.8, 6.5 Hz, 2H). |
| 424 | ** | ** | 476.3 | 1H NMR(400 MHz, DMSO-d6) δ 8.33 (s, 1H), 8.06 |
| | | | | (d, J = 2.1 Hz, 1H), 7.92-7.86 (m, 1H), 7.53-7.43 |
| | | | | (m, 2H), 4.80-4.71 (m, 1H), 4.62 (d, J = 15.9 Hz, |
| | | | | 2H), 3.92 (d, J = 1.5 Hz, 3H), 3.84-3.74 (m, 2H), |
| | | | | 3.32-3.27 (m, 2H), 3.00 (t, J = 9.9 Hz, 2H), 2.91 (t, |
| | | | | J = 5.7 Hz, 1H), 2.77 (t, J = 5.5 Hz, 1H), 2.12 (d, J = |
| | | | | 4.8 Hz, 5H), 1.93-1.79 (m, 2H). |
| 425 | ** | ** | 463.3 | 1H NMR(400 MHz, DMSO-d6) δ 8.32 (s, 1H), 8.05 |
| | | | | (d, J = 1.8 Hz, 1H), 6.97-6.90 (m, 2H), 6.88-6.83 |
| | | | | (m, 2H), 4.62 (d, J = 16.2 Hz, 2H), 4.46-4.38 (m, |
| | | | | 1H), 3.92 (d, J = 1.7 Hz, 3H), 3.81-3.76 (m, 2H), |
| | | | | 3.69 (s, 3H), 3.31-3.27 (m, 2H), 3.00-2.87 (m, |
| | | | | 3H), 2.76 (t, J = 5.7 Hz, 1H), 2.12 (d, J = 5.0 Hz, 3H), |
| | | | | 2.09-2.00 (m, 2H), 1.84-1.74 (m, 2H). |
| 426 | ** | ** | 493.4 | 1H NMR(400 MHz, DMSO-d6) δ 8.31 (s, 1H), 8.05 |
| | | | | (d, J = 1.6 Hz, 1H), 6.93 (d, J = 8.7 Hz, 1H), 6.58 (d, |
| | | | | J = 2.8 Hz, 1H), 6.41 (dd, J = 8.7, 2.8 Hz, 1H), 4.62 (d, |
| | | | | J = 16.0 Hz, 2H), 4.30-4.18 (m, 1H), 3.92 (d, J = 1.8 |
| | | | | Hz, 3H), 3.83-3.73 (m, 6H), 3.71 (s, 3H), 2.90 (t, J = |
| | | | | 8.7 Hz, 4H), 2.79-2.73 (m, 1H), 2.12 (d, J = 5.0 Hz, |
| | | | | 3H), 1.98 (s, 2H), 1.79 (dd, J = 17.7, 8.8 Hz, 2H). |
| 427 | ** | ** | 481.2 | 1H NMR(400 MHz, DMSO-d6) δ 8.32 (s, 1H), 8.05 |
| | | | | (s, 1H), 7.07-7.01 (m, 1H), 6.85 (t, J = 7.6 Hz, 1H), |
| | | | | 6.77 (t, J = 7.6 Hz, 1H), 4.60 (t, J = 17.4 Hz, 3H), |
| | | | | 3.92 (s, 3H), 3.86-3.75 (m, 5H), 3.30-3.25 (m, |
| | | | | 2H), 3.02-2.87 (m, 3H), 2.77 (d, J = 5.0 Hz, 1H), |
| | | | | 2.16-2.01 (m, 5H), 1.84 (d, J = 9.0 Hz, 2H). |
| 428 | *** | ** | 487.2 | 1H NMR(400 MHz, DMSO-d6) δ 8.31 (s, 1H), 8.05 |
| | | | | (d, J = 1.4 Hz, 1H), 7.29 (dd, J = 17.7, 8.8 Hz, 2H), |
| | | | | 4.61 (d, J = 15.8 Hz, 2H), 4.24 (s, 1H), 3.92 (d, J = |
| | | | | 1.7 Hz, 3H), 3.84-3.75 (m, 2H), 3.34 (s, 3H), 2.96- |
| | | | | 2.87 (m, 3H), 2.76 (t, J = 5.6 Hz, 1H), 2.08 (dd, J = |
| | | | | 25.7, 11.2 Hz, 6H), 1.86 (dd, J = 11.0, 6.4 Hz, 2H). |
| 429 | *** | ** | 501.2 | 1H NMR(400 MHz, DMSO-d6) δ 8.32 (s, 1H), 8.05 |
| | | | | (d, J = 2.0 Hz, 1H), 7.58 (d, J = 2.5 Hz, 1H), 7.36 (dd, |
| | | | | J = 8.9, 2.5 Hz, 1H), 7.29 (d, J = 9.0 Hz, 1H), 4.69 (s, |
| | | | | 1H), 4.62 (d, J = 15.6 Hz, 2H), 3.92 (d, J = 1.5 Hz, |
| | | | | 3H), 3.82-3.76 (m, 2H), 3.08-2.98 (m, 2H), 2.90 (t, |
| | | | | J = 5.7 Hz, 1H), 2.80-2.74 (m, 1H), 2.16-2.05 (m, |
| | | | | 5H), 1.85 (d, J = 9.0 Hz, 2H). |
| 430 | **** | *** | 501.2 | 1H NMR(400 MHz, DMSO-d6) δ 8.32 (s, 1H), 8.05 |
| | | | | (d, J = 2.0 Hz, 1H), 7.46 (dd, J = 8.5, 4.7 Hz, 1H), |
| | | | | 7.39 (d, J = 2.3 Hz, 1H), 7.03 (dd, J = 8.5, 2.3 Hz, |
| | | | | 1H), 4.78 (s, 1H), 4.62 (d, J = 15.6 Hz, 2H), 3.92 (s, |
| | | | | 3H), 3.79 (d, J = 4.2 Hz, 2H), 3.31-3.22 (m, 2H), |
| | | | | 3.09-3.00 (m, 2H), 2.91 (d, J = 5.6 Hz, 1H), 2.77 (s, |
| | | | | 1H), 2.16-2.04 (m, 5H), 1.85 (d, J = 9.2 Hz, 2H). |
| 431 | ** | ** | 469.2 | 1H NMR(400 MHz, DMSO-d6) δ 8.32 (s, 1H), 8.05 |
| | | | | (d, J = 1.7 Hz, 1H), 6.85-6.71 (m, 3H), 4.62 (d, J = |
| | | | | 16.1 Hz, 3H), 3.92 (s, 3H), 3.85-3.74 (m, 2H), 3.27- |
| | | | | 3.23 (m, 2H), 2.99 (t, J = 10.5 Hz, 2H), 2.90 (t, J = |
| | | | | 5.6 Hz, 1H), 2.77 (t, J = 5.6 Hz, 1H), 2.17-2.02 (m, |
| | | | | 5H), 1.88-1.74 (m, 2H). |
| 432 | ** | ** | 465.3 | 1H NMR(400 MHz, DMSO-d6) δ 8.32 (s, 1H), 8.06 |
| | | | | (d, J = 1.9 Hz, 1H), 7.02 (dd, J = 8.7, 4.5 Hz, 2H), |
| | | | | 6.94 (td, J = 8.6, 3.0 Hz, 1H), 4.62 (d, J = 15.5 Hz, |
| | | | | 2H), 4.49 (bs, 1H), 3.92 (s, 3H), 3.84-3.73 (m, 2H), |
| | | | | 3.29-3.22 (m, 2H), 3.07-2.94 (m, 2H), 2.90 (t, J = |
| | | | | 5.5 Hz, 1H), 2.76 (t, J = 5.4 Hz, 1H), 2.18 (s, 3H), |
| | | | | 2.15-2.01 (m, 5H), 1.81 (m, 2H). |
| 433 | ** | ** | 452.5 | 1H NMR(400 MHz, DMSO-d6) δ 8.67 (s, 1H), 8.35 |
| | | | | (s, 1H), 8.04 (s, 1H), 7.35-7.26 (m, 2H), 7.06-6.98 |
| | | | | (m, 1H), 6.78-6.75 (m, 1H), 6.35 (s, 1H), 4.58-4.54 |
| | | | | (m, 1H), 3.93 (s, 3H), 3.60-3.51 (m, 2H), 3.18- |
| | | | | 3.13 (m, 2H), 2.83 (d, J = 4.9 Hz, 3H), 2.09-2.05 (m, |
| | | | | 2H), 1.88-1.77 (m, 2H). |
| 434 | *** | ** | 425.4 | 1H NMR(400 MHz, DMSO-d6) δ 9.05 (d, J = 0.6 Hz, |
| | | | | 1H), 8.50 (s, 1H), 8.15 (d, J = 0.5 Hz, 1H), 7.87 (d, |
| | | | | J = 0.6 Hz, 1H), 7.38-7.24 (m, 2H), 7.06-6.98 (m, |
| | | | | 1H), 5.36 (s, 1H), 5.03 (s, 1H), 4.63-4.53 (m, 1H), |
| | | | | 3.96 (s, 3H), 3.74-3.59 (overlapped m, 2H), 3.31- |
| | | | | 3.20 (m, 2H), 2.14-2.02 (m, 2H), 1.92-1.77 (m, |
| | | | | 2H), 1.54 (s, 3H), 1.12 (s, 3H), 0.99 (s, 3H). |
| 435 | *** | ** | 481.5 | 1H NMR(400 MHz, DMSO-d6) δ 8.79 (dd, J = 5.2, |
| | | | | 2.9 Hz, 1H), 7.75 (dd, J = 5.2, 1.5 Hz, 1H), 7.33-7.22 |
| | | | | (m, 2H), 7.03-6.94 (m, 1H), 4.69-4.58 (m, 2H), |
| | | | | 4.55-4.45 (m, 1H), 3.85-3.73 (m, 2H), 3.51-3.39 |
| | | | | (m, 2H), 3.14-3.00 (m, 2H), 2.94 (t, J = 5.9 Hz, 1H), |
| | | | | 2.81 (t, J = 5.6 Hz, 1H), 2.66 (s, 3H), 2.13 (s, 1.6H), |
| | | | | 2.11 (s, 1.4H), 1.96-1.84 (m, 2H), 1.69-1.54 (m, 2H). |
| 436 | ** | ** | 448.3 | 1H NMR(400 MHz, DMSO-d6) δ 9.20 (s, 1H), 8.96 |
| | | | | (s, 1H), 8.46 (s, 1H), 8.09 (s, 1H), 7.34-7.21 (m, |
| | | | | 2H), 7.01-6.91 (m, 1H), 4.62-4.50 (m, 1H), 3.92 |
| | | | | (s, 3H), 3.83-3.70 (m, 2H), 3.29-3.22 (overlapping |
| | | | | m, 2H), 2.10-1.99 (m, 2H), 1.86-1.72 (m, 2H). |
| 437 | *** | ** | 497.4 | 1H NMR(400 MHz, DMSO-d6) δ 9.05 (s, 1H), 8.50 |
| | | | | (s, 1H), 8.15 (s, 1H), 7.68 (s, 1H), 7.40-7.20 (m, |
| | | | | 2H), 7.02 (t, J = 8.5 Hz, 1H), 5.63 (bs, 1H), 4.98- |
| | | | | 4.80 (m, 1H), 4.68-4.47 (m, 1H), 3.95 (s, 3H), 3.72 |
| | | | | (overlapping dd, J = 10.1, 3.3 Hz, 1H), 3.69-3.60 |
| | | | | (overlapping m, 2H), 3.56 (overlapping dd, J = 10.0, |
| | | | | 6.9 Hz, 1H), 3.28 (overlapping s, 3H), 3.27-3.19 |
| | | | | (overlapping m, 2H), 2.16-2.00 (m, 2H), 1.93-1.71 |
| | | | | (m, 2H). |
| 438 | *** | ** | 497.4 | 1H NMR(400 MHz, DMSO-d6) δ 9.08 (d, J = 0.6 Hz, |
| | | | | 1H), 8.50 (s, 1H), 8.16 (d, J = 0.5 Hz, 1H), 7.55 (s, |
| | | | | 1H), 7.37-7.25 (m, J = 8.8, 8.1, 4.3 Hz, 2H), 7.06- |
| | | | | 6.97 (m, 1H), 4.95 (bs, 1H), 4.63-4.52 (m, 1H), 4.47- |
| | | | | 4.35 (m, 1H), 3.95 (s, 3H), 3.73 (dd, J = 11.5, 3.8 |
| | | | | Hz, 1H), 3.68-3.59 (m, 3H), 3.32 (overlapping s, |
| | | | | 3H), 3.27-3.22 (overlapping m, 2H), 2.15-1.99 (m, |
| | | | | 2H), 1.93-1.74 (m, 2H). |
| 439 | **** | ** | 487.3 | 1H NMR(400 MHz, DMSO-d6) δ 8.72 (d, 1H), 8.26 |
| | | | | (d, J = 3.8 Hz, 1H), 7.37-7.23 (m, 2H), 7.07-6.95 |
| | | | | (m, 1H), 6.32 (d, J = 1.9 Hz, 1H), 6.19 (d, J = 2.0 Hz, |
| | | | | 1H), 4.65 (d, J = 16.6 Hz, 2H), 4.55-4.45 (m, 1H), |
| | | | | 3.85-3.74 (m, 2H), 3.33-3.26 (overlapping m, 2H), |
| | | | | 3.02-2.89 (m, 3H), 2.79 (t, J = 5.8 Hz, 1H), 2.13 |
| | | | | (overlapping s, 1.5H), 2.12 (overlapping s, 1.5H), |
| | | | | 2.10-2.01 (overlapping m, 2H), 1.92-1.78 (m, 2H). |
| 440 | ** | ** | 484.3 | 1H NMR(400 MHz, DMSO-d6) δ 8.61 (s, 1H), 8.37 |
| | | | | (s, 1H), 8.03 (s, 1H), 7.47 (s, 0.5H), 7.45 (s, 0.5H), |
| | | | | 7.40 (d, J = 2.3 Hz, 1H), 7.04 (d, J = 2.3 Hz, 0.5H), |
| | | | | 7.02 (d, J = 2.3 Hz, 0.5H), 6.46 (s, 1H), 6.23 (s, 1H), |
| | | | | 4.87-4.77 (m, 1H), 4.23 (q, J = 7.2 Hz, 2H), 3.57- |
| | | | | 3.44 (m, 2H), 3.28-3.18 (m, 2H), 2.13-2.01 (m, |
| | | | | 2H), 1.89-1.76 (m, 2H), 1.42 (t, J = 7.3 Hz, 3H) |
| 441 | *** | ** | 557.5 | 1H NMR(400 MHz, DMSO-d6) δ 9.03-8.92 (m, |
| | | | | 1H), 8.57-8.43 (m, 1H), 8.18-8.07 (m, 1H), 7.72- |
| | | | | 7.61 (m, 1H), 7.45-7.40 (m, 1H), 7.39-7.34 (m, |
| | | | | 1H), 7.04-6.95 (m, 1H), 5.52-5.38 (m, 1H), 4.88- |
| | | | | 4.76 (m, 1H), 4.77-4.68 (m, 1H), 4.54-4.42 (m, |
| | | | | 1H), 4.22 (q, J = 6.9 Hz, 2H), 3.64-3.51 (m, 2H), |
| | | | | 3.40-3.28 (overlapping m, 2H), 2.15-1.97 (m, 2H), |
| | | | | 1.92-1.77 (m, 2H), 1.40 (t, J = 7.1 Hz, 3H), 1.05 (s, |
| | | | | 3H), 1.02 (s, 3H); |
| 442 | ** | ** | 496.3 | 1H NMR(400 MHz, DMSO-d6) δ 9.07 (s, 1H), 8.25 |
| | | | | (s, 1H), 8.13 (s, 1H), 7.62 (s, 1H), 7.16 (td, J = 9.3, |
| | | | | 5.5 Hz, 1H), 7.06-6.94 (m, 1H), 6.95-6.82 (m, |
| | | | | 1H), 4.58-4.44 (m, 1H), 4.27-4.10 (m, 1H), 3.94 |
| | | | | (overlapping s, 3H), 3.93-3.89 (overlapping m, 1H), |
| | | | | 3.87-3.76 (m, 1H), 3.75-3.63 (m, 2H), 3.36-3.22 |
| | | | | (m, 2H), 2.51 (overalapping s, 3H), 2.14-2.04 (m, |
| | | | | 2H), 1.91-1.81 (m, 2H). |
| 443 | ** | ** | 470.3 | 1H NMR (400 MHz, DMSO-d6) δ 8.63-8.60 (m, |
| | | | | 1H), 8.33 (s, 1H), 8.05-8.01 (m, 1H), 7.47 (s, 0.5H), |
| | | | | 7.45 (s, 0.5H), 7.40 (d, J = 2.3 Hz, 1H), 7.04 (d, J = |
| | | | | 2.3 Hz, 0.5H), 7.02 (d, J = 2.3 Hz, 0.5H), 6.46 (d, J = |
| | | | | 0.6 Hz, 1H), 6.22 (br. s, 2H), 4.89-4.72 (m, 1H), |
| | | | | 3.93 (s, 3H), 3.58-3.43 (m, 2H), 3.30-3.14 (m, |
| | | | | 2H), 2.15-2.00 (m, 2H), 1.94-1.72 (m, 2H) |
| 444 | ** | ** | 481.5 | 1H NMR(400 MHz, DMSO-d6) δ 8.89 (d, J = 1.5 Hz, |
| | | | | 1H), 8.54 (d, J = 1.9 Hz, 1H), 7.32-7.20 (m, 2H), |
| | | | | 7.03-6.92 (m, 1H), 4.71-4.58 (m, 2H), 4.53-4.42 |
| | | | | (m, 1H), 3.90-3.74 (m, 2H), 3.47-3.36 (m, 2H), |
| | | | | 3.07-2.98 (m, 2H), 2.94 (t, J = 5.9 Hz, 1H), 2.81 (t, |
| | | | | J = 5.8 Hz, 1H), 2.55 (s, 3H), 2.14 (s, 1.6H), 2.11 (s, |
| | | | | 1.4H), 1.93-1.80 (m, 2H), 1.65-1.49 (m, 2H). |
| 445 | *** | ** | 483.3 | 1H NMR(400 MHz, DMSO-d6) δ 8.32 (s, 1H), 8.05 |
| | | | | (s, 1H), 7.34-7.25 (m, 2H), 7.05-6.97 (m, 1H), |
| | | | | 5.17-5.04 (m, 1H), 4.74 (d, J = 17.1 Hz, 0.5H), 4.55- |
| | | | | 4.44 (m, 2H), 4.03 (d, J = 18.2 Hz, 0.5H), 3.92 (s, |
| | | | | 3H), 3.27-3.11 (overlapping m, 3H), 3.02-2.94 (m, |
| | | | | 2H), 2.67-2.56 (m, 1H), 2.14 (s, 1.6H), 2.11 (s, |
| | | | | 1.4H), 2.10-2.02 (m, 2H), 1.91-1.77 (m, 2H), 1.16 |
| | | | | (d, J = 6.8 Hz, 1.6H), 1.03 (d, J = 6.9 Hz, 1.6H). |
| 446 | *** | ** | 483.4 | 1H NMR(400 MHz, DMSO-d6) δ 8.30 (s, 1H), 8.05 |
| | | | | (s, 1H), 7.34-7.22 (m, 2H), 7.06-6.94 (m, 1H), |
| | | | | 5.15-5.05 (m, 1H), 4.74 (d, J = 17.3 Hz, 0.5H), 4.55- |
| | | | | 4.45 (m, 2H), 4.00 (d, J = 18.2 Hz, 0.5H), 3.92 (s, |
| | | | | 3H), 3.26-3.12 (overlapping m, 3H), 2.99-2.88 (m, |
| | | | | 2H), 2.68-2.56 (m, 2H), 2.14 (s, 1.6H), 2.11 (s, |
| | | | | 1.4H), 2.09-2.00 (m, 2H), 1.89-1.78 (m, 2H), 1.16 |
| | | | | (d, J = 6.7 Hz, 1.6H), 1.03 (d, J = 7.0 Hz, 1.6H). |
| 447 | ** | ** | 453.4 | 1H NMR(400 MHz, DMSO-d6) δ 8.63 (s, 1H), 8.27 |
| | | | | (s, 1H), 7.94 (s, 1H), 7.36-7.23 (m, 2H), 7.08-6.95 |
| | | | | (m, 1H), 6.18 (s, 1H), 4.62-4.46 (m, 1H), 3.92 |
| | | | | (overlapping s, 3H), 3.62 (overlapping s, 3H), 3.60- |
| | | | | 3.55 (overlapping m, 1H), 3.30-3.28 (overlapping |
| | | | | m, 1H), 3.28-3.15 (m, 2H), 2.09-1.95 (m, 2H), |
| | | | | 1.86-1.69 (m, 2H). |
| 448 | *** | ** | 483.5 | 1H NMR(400 MHz, DMSO-d6) δ 7.81 (t, J = 2.3 Hz, |
| | | | | 1H), 7.31-7.24 (m, 2H), 7.03-6.96 (m, 1H), 6.74 |
| | | | | (d, J = 2.2 Hz, 1H), 4.64 (s, 0.92H), 4.59 (s, 1.10H, |
| | | | | rotamers), 4.52-4.44 (m, 1H), 4.20 (q, J = 7.3 Hz, |
| | | | | 2H), 3.79 (q, J = 5.7 Hz, 2H), 3.47-3.39 (m, 2H), |
| | | | | 3.03-2.95 (m, 2H), 2.90 (t, J = 5.8 Hz, 1H), 2.77 (t, |
| | | | | J = 5.8 Hz, 1H), 2.13 (s, 1.63H), 2.11 (s, 1.38H, |
| | | | | rotamers), 2.02-1.93 (m, 2H), 1.78-1.66 (m, 2H), |
| | | | | 1.42 (t, J = 7.3 Hz, 3H). |
| 449 | ** | ** | 508.4 | 1H NMR(400 MHz, DMSO-d6) δ 8.79 (s, 1H), 8.39 |
| | | | | (s, 1H), 8.07 (s, 1H), 7.36-7.26 (m, 2H), 7.14-6.91 |
| | | | | (m, 1h), 6.78 (s, 1h), 4.67-4.47 (m, 1H), 3.93 (s, |
| | | | | 3H), 3.79-3.66 (m, 4H), 3.64-3.45 (m, 6H), 3.22- |
| | | | | 3.08 (m, 2H), 2.14-1.99 (m, 2H), 1.89-1.73 (m, |
| | | | | 2H) ppm. |
| 450 | ** | ** | 508.4 | 1H NMR(400 MHz, DMSO-d6) δ 8.73 (d, J = 0.4 Hz, |
| | | | | 1H), 8.37 (s, 1H), 8.05 (s, 1H), 7.35-7.23 (m, 2H), |
| | | | | 7.07-6.96 (m, 1H), 6.38 (s, 1H), 4.60-4.51 (m, |
| | | | | 1H), 4.40-4.32 (m, 1H), 4.23 (dd, J = 9.1, 6.3 Hz, |
| | | | | 2H), 3.93 (s, 3H), 3.84 (dd, J = 9.4, 4.0 Hz, 2H), 3.63- |
| | | | | 3.52 (m, 2H), 3.27 (s, 3H), 3.22-3.13 (m, 2H), |
| | | | | 2.11-2.00 (m, 2H), 1.86-1.77 (m, 2H) ppm. |
| 451 | * | ** | 325.2 | 1H NMR(400 MHz, DMSO-d6) d 7.97 (t, J = 4.5 Hz, |
| | | | | 1H), 7.65 (s, 1H), 5.87 (s, 2H), 4.56 (d, J = 17.2 Hz, |
| | | | | 2H), 4.06 (s, 4H), 3.89 (s, 4H), 3.77 (dd, J = 11.0, 4.9 |
| | | | | Hz, 2H), 3.17 (s, 1H), 2.91-2.83 (m, 1H), 2.78- |
| | | | | 2.70 (m, 1H), 2.12 (t, J = 12.8 Hz, 3H). |
| 452 | *** | ** | 481.2 | 1H NMR(400 MHz, DMSO-d6) δ 8.32 (s, 1H), 8.05 |
| | | | | (d, J = 1.7 Hz, 1H), 7.17 (t, J = 9.4 Hz, 1H), 6.88 (dd, |
| | | | | J = 13.0, 3.0 Hz, 1H), 6.72-6.67 (m, 1H), 4.62 (d, |
| | | | | J = 16.0 Hz, 2H), 4.37 (s, 1H), 3.92 (d, J = 1.7 Hz, 3H), |
| | | | | 3.79 (dd, J = 11.7, 7.6 Hz, 2H), 3.72 (s, 3H), 3.32 (s, |
| | | | | 2H), 2.93 (dd, J = 17.1, 7.8 Hz, 3H), 2.77 (d, J = 5.6 |
| | | | | Hz, 1H), 2.12 (d, J = 4.9 Hz, 3H), 2.02 (s, 2H), 1.91- |
| | | | | 1.75 (m, 2H). |
| 453 | ** | ** | 481.2 | 1H NMR(400 MHz, DMSO-d6) δ 8.32 (s, 1H), 8.05 |
| | | | | (d, J = 1.7 Hz, 1H), 7.34 (dd, J = 19.8, 9.4 Hz, 1H), |
| | | | | 7.15 (ddd, J = 12.8, 6.8, 3.0 Hz, 1H), 6.86-6.79 (m, |
| | | | | 1H), 4.67-4.52 (m, 3H), 3.92 (d, J = 1.6 Hz, 3H), |
| | | | | 3.83-3.75 (m, 2H), 3.32 (s, 2H), 2.97 (t, J = 10.6 Hz, |
| | | | | 2H), 2.92 (s, 1H), 2.90 (t, J = 5.7 Hz, 1H), 2.77 (t, J = |
| | | | | 5.7 Hz, 1H), 2.10 (dd, J = 20.2, 8.5 Hz, 5H), 1.87- |
| | | | | 1.75 (m, 2H). |
| 454 | ** | ** | 469.2 | 1H NMR(400 MHz, DMSO-d6) δ 8.31 (s, 1H), 8.05 |
| | | | | (d, J = 1.5 Hz, 1H), 7.19-7.13 (m, 3H), 4.62 (d, J = |
| | | | | 15.8 Hz, 2H), 4.31 (d, J = 3.6 Hz, 1H), 3.92 (d, J = |
| | | | | 1.8 Hz, 3H), 3.82-3.75 (m, 2H), 3.35 (d, J = 16.1 |
| | | | | Hz, 2H), 2.92 (dd, J = 12.2, 7.9 Hz, 3H), 2.76 (t, J = |
| | | | | 5.6 Hz, 1H), 2.12 (d, J = 5.1 Hz, 3H), 2.05 (d, J = |
| | | | | 12.4 Hz, 2H), 1.92-1.82 (m, 2H). |
| 455 | ** | ** | 487.2 | 1H NMR(400 MHz, DMSO-d6) δ 8.32 (s, 1H), 8.05 |
| | | | | (d, J = 1.5 Hz, 1H), 7.31-7.16 (m, 2H), 4.62 (d, J = |
| | | | | 15.8 Hz, 2H), 4.40 (s, 1H), 3.92 (d, J = 1.7 Hz, 3H), |
| | | | | 3.83-3.74 (m, 2H), 3.33 (s, 2H), 2.99-2.86 (m, |
| | | | | 3H), 2.76 (t, J = 5.6 Hz, 1H), 2.15-2.03 (m, 5H), |
| | | | | 1.95-1.82 (m, 2H). |
| 456 | *** | ** | 485.2 | 1H NMR(400 MHz, DMSO-d6) δ 8.32 (s, 1H), 8.05 |
| | | | | (d, J = 1.9 Hz, 1H), 7.48-7.47 (m, 1H), 7.44 (dd, J = |
| | | | | 11.1, 2.5 Hz, 1H), 7.31 (t, J = 9.0 Hz, 1H), 4.62 (d, |
| | | | | J = 16.0 Hz, 3H), 3.92 (d, J = 1.6 Hz, 3H), 3.84-3.74 |
| | | | | (m, 2H), 3.32 (s, 2H), 2.94 (dt, J = 11.2, 7.7 Hz, 3H), |
| | | | | 2.77 (t, J = 5.6 Hz, 1H), 2.16-2.04 (m, 5H), 1.90- |
| | | | | 1.78 (m, 2H). |
| 457 | *** | ** | 485.2 | 1H NMR(400 MHz, DMSO-d6) δ 8.33 (s, 1H), 8.05 |
| | | | | (d, J = 1.9 Hz, 1H), 7.41 (dd, J = 7.4, 2.5 Hz, 1H), |
| | | | | 7.27 (dd, J = 11.1, 8.7 Hz, 1H), 7.01 (ddd, J = 8.7, |
| | | | | 3.8, 2.6 Hz, 1H), 4.68 (dd, J = 7.8, 4.1 Hz, 1H), 4.62 |
| | | | | (d, J = 16.0 Hz, 2H), 3.92 (d, J = 1.6 Hz, 3H), 3.84- |
| | | | | 3.75 (m, 2H), 3.32-3.26 (m, 2H), 2.99 (t, J = 10.0 |
| | | | | Hz, 2H), 2.90 (t, J = 5.6 Hz, 1H), 2.77 (t, J = 5.7 Hz, |
| | | | | 1H), 2.15-2.04 (m, 5H), 1.92-1.79 (m, 2H). |
| 458 | *** | ** | 503.2 | 1H NMR(400 MHz, DMSO-d6) δ 8.32 (s, 1H), 8.05 |
| | | | | (d, J = 1.8 Hz, 1H), 7.64-7.52 (m, 2H), 4.68-4.57 |
| | | | | (m, 3H), 3.92 (d, J = 1.5 Hz, 3H), 3.82-3.76 (m, |
| | | | | 2H), 3.02-2.93 (m, 2H), 2.90 (t, J = 5.7 Hz, 1H), |
| | | | | 2.77 (t, J = 5.6 Hz, 1H), 2.15-2.03 (m, 5H), 1.90- |
| | | | | 1.78 (m, 2H). |
| 459 | *** | ** | 438.4 | 1H NMR (400 MHz, DMSO-d6) δ 8.49 (s, 1H), 8.37 |
| | | | | (s, 1H), 8.16 (s, 1H), 7.97 (s, 1H), 7.38-7.26 (m, |
| | | | | 2H), 7.06-6.95 (m, 1H), 5.73 (s, 2H), 4.62-4.55 |
| | | | | (m, 1H), 3.95 (s, 3H), 3.65-3.57 (m, 2H), 3.25- |
| | | | | 3.18 (m, 2H), 2.14-2.02 (m, 2H), 1.86 (m, 2H). |
| 460 | *** | ** | 474.3 | 1H NMR(400 MHz, DMSO-d6) δ 8.80 (s, 1H), 8.66 |
| | | | | (d, J = 0.6 Hz, 1H), 8.36 (s, 1H), 7.93 (t, J = 58.8 Hz, |
| | | | | 1H), 7.35-7.25 (m, 2H), 7.07-6.96 (m, 1H), 6.47 |
| | | | | (s, 1H), 6.36 (s, 2H), 4.60-4.50 (m, 1H), 3.59-3.48 |
| | | | | (m, 2H), 3.19-3.07 (m, 2H), 2.11-1.99 (m, 2H), |
| | | | | 1.87-1.75 (m, 2H). |
| 461 | *** | ** | 495.5 | 1H NMR(400 MHz, DMSO-d6) δ 9.04 (s, 1H), 8.49 |
| | | | | (s, 1H), 8.15 (s, 1H), 7.53 (s, 1H), 7.36-7.25 (m, |
| | | | | 2H), 7.08-6.96 (m, 1H), 4.76 (s, 1H), 4.63-4.52 |
| | | | | (m, 1H), 3.95 (s, 3H), 3.69-3.58 (m, 2H), 3.29- |
| | | | | 3.19 (overlapped m, 2H), 2.97 (s, 2H), 2.15-2.02 (m, |
| | | | | 2H), 1.91-1.77 (m, 2H), 1.13 (s, 6H). |
| 462 | *** | ** | 464.3 | 1H NMR(400 MHz, DMSO-d6) δ 8.61 (d, J = 0.5 Hz, |
| | | | | 1H), 8.37 (s, 1H), 8.01 (d, J = 0.5 Hz, 1H), 7.36- |
| | | | | 7.24 (m, 2H), 7.07-6.94 (m, 1H), 6.45 (d, J = 0.6 |
| | | | | Hz, 1H), 6.24 (s, 2H), 4.61-4.48 (m, 1H), 3.86 (tt, |
| | | | | J = 7.4, 3.9 Hz, 1H), 3.61-3.47 (m, 2H), 3.20-3.07 |
| | | | | (m, 2H), 2.10-1.97 (m, 2H), 1.86-1.72 (m, 2H), |
| | | | | 1.15-1.09 (m, 2H), 1.04-0.97 (m, 2H). |
| 463 | *** | ** | 473.5 | 1H NMR(400 MHz, DMSO-d6) δ 7.36-7.26 (m, |
| | | | | 2H), 7.06-6.98 (m, 1H), 4.59-4.45 (m, 3H, |
| | | | | overlapped), 3.83-3.67 (m, 2H), 3.61-3.54 (m, |
| | | | | 1H), 3.52 (dd, J = 10.3, 5.7 Hz, 1H), 3.46-3.39 (m, |
| | | | | 1H), 3.29-3.24 (m, 1H), 3.24 (s, 1.15H), 3.23 (s, |
| | | | | 1.77H, rotamers), 3.13 (t, J = 9.7 Hz, 1H), 3.04-2.96 |
| | | | | (m, 1H), 2.86-2.81 (m, 1H), 2.74-2.68 (m, 1H), |
| | | | | 2.14-2.01 (m, 6H, overlapped), 1.88-1.76 (m, 2H), |
| | | | | 1.64-1.54 (m, 1H), 1.29-1, 21 (m, 1H), 1.01-0.91 |
| | | | | (m, 1H) |
| 464 | *** | ** | 522.4 | 1H NMR(400 MHz, DMSO-d6) δ 9.07 (d, J = 0.5 Hz, |
| | | | | 1H), 8.50 (s, 1H), 8.22 (d, J = 4.1 Hz, 1H), 8.15 (s, |
| | | | | 1H), 7.67 (s, 1H), 7.36-7.24 (m, 2H), 7.05-6.96 |
| | | | | (m, 1H), 5.00 (dd, J = 10.2, 3.1 Hz, 1H), 4.64-4.52 |
| | | | | (m, 1H), 4.33-4.23 (m, 2H), 3.95 (s, 3H), 3.71- |
| | | | | 3.59 (m, 3H), 3.44 (dd, J = 11.9, 10.6 Hz, 1H), 3.30- |
| | | | | 3.23 (m, 2H), 2.14-2.00 (m, 2H), 1.90-1.76 (m, |
| | | | | 2H). Rotamers present |
| 465 | *** | ** | 482.4 | 1H NMR(400 MHz, DMSO-d6) δ 8.07 (d, J = 3.2 Hz, |
| | | | | 1H), 7.85 (s, 1H), 7.29-7.21 (m, 2H), 7.02-6.94 |
| | | | | (m, 1H), 6.59 (s, 2H), 4.63 (s, 0.88H), 4.58 (s, 1.07H, |
| | | | | rotamers), 4.51-4.43 (m, 1H), 3.79 (q, J = 5.6 Hz, |
| | | | | 2H), 3.47-3.38 (m, 2H), 3.05-2.96 (m, 2H), 2.91 (t, |
| | | | | J = 5.7 Hz, 1H), 2.78 (t, J = 5.8 Hz, 1H), 2.13 (s, |
| | | | | 1.61H), 2.10 (s, 1.29H, rotamers), 1.89 (m, 2H), 1.65- |
| | | | | 1.54 (m, 2H). |
| 466 | ** | ** | 501.4 | 1H NMR(400 MHz, DMSO-d6) δ 8.90-8.80 (m, |
| | | | | 1H), 8.10-8.01 (m, 1H), 7.36-7.22 (m, 2H), 7.06- |
| | | | | 6.94 (m, 1H), 4.73-4.60 (m, 2H), 4.59-4.48 (m, |
| | | | | 1H), 3.88-3.74 (m, 2H), 3.59-3.41 (m, 2H), 3.23- |
| | | | | 3.04 (m, 2H), 2.96 (t, 1H), 2.82 (t, 1H), 2.14 (s, |
| | | | | 1.6H), 2.11 (s, 1.4H), 1.96 (m, 2H), 1.81-1.59 (m, 2H). |
| 467 | ** | ** | 467.4 | 1H NMR(400 MHz, DMSO-d6) δ 9.04 (s, 1H), 8.50 |
| | | | | (s, 1H), 8.15 (s, 1H), 7.67 (s, 1H), 7.38-7.23 (m, |
| | | | | 2H), 7.07-6.96 (m, 1H), 5.47 (d, J = 4.8 Hz, 1H), |
| | | | | 4.91-4.79 (m, 1H), 4.62-4.52 (m, 1H), 3.95 (s, |
| | | | | 3H), 3.72-3.58 (m, 2H), 3.30-3.14 (m, 2H), 2.13- |
| | | | | 2.02 (m, 2H), 1.90-1.77 (m, 2H), 1.49-1.39 (m, 3H). |
| 468 | *** | ** | 467.5 | 1H NMR(400 MHz, DMSO-d6) δ 9.04 (s, 1H), 8.49 |
| | | | | (s, 1H), 8.15 (s, 1H), 7.49 (s, 1H), 7.37-7.23 (m, |
| | | | | 2H), 7.08-6.96 (m, 1H), 4.68 (t, J = 5.3 Hz, 1H), |
| | | | | 4.63-4.53 (m, 1H), 3.95 (s, 3H), 3.86-3.77 (m, |
| | | | | 2H), 3.68-3.57 (m, 2H), 3.28-3.18 (m, 2H), 3.01 (t, |
| | | | | J = 6.7 Hz, 2H), 2.13-2.02 (m, 2H), 1.89-1.76 (m, 2H). |
| 469 | *** | ** | 537.4 | 1H NMR(400 MHz, DMSO-d6) δ 9.00 (s, 1H), 8.50 |
| | | | | (s, 1H), 8.10 (s, 1H), 7.66 (s, 1H), 7.34-7.21 (m, |
| | | | | 2H), 7.03-6.94 (m, 1H), 5.45 (d, J = 5.4 Hz, 1H), |
| | | | | 4.73 (s, 1H), 4.58-4.50 (m, 1H), 4.47 (d, J = 5.4 Hz, |
| | | | | 1H), 3.87 (tt, J = 7.5, 3.9 Hz, 1H), 3.65-3.55 (m, |
| | | | | 2H), 3.27-3.15 (overlapped m, 2H), 2.10-1.97 (m, |
| | | | | 2H), 1.86-1.71 (m, 2H), 1.14-1.08 (m, 2H), 1.04 |
| | | | | (s, 3H), 1.01 (s, 3H), 1.01-0.95 (overlapped m, 2H). |
| 470 | ** | ** | 483.4 | 1H NMR(400 MHz, DMSO-d6) δ 9.04 (s, 1H), 8.50 |
| | | | | (s, 1H), 8.16 (s, 1H), 7.67 (s, 1H), 7.38-7.24 (m, |
| | | | | 2H), 7.07-6.98 (m, 1H), 5.51 (d, J = 5.1 Hz, 1H), |
| | | | | 4.77-4.69 (m, 2H), 4.63-4.53 (m, 1H), 3.95 (s, |
| | | | | 3H), 3.82-3.73 (m, 1H), 3.69-3.61 (m, 2H), 3.59- |
| | | | | 3.49 (m, 1H), 3.29-3.18 (m, 2H), 2.13-2.03 (m, |
| | | | | 2H), 1.88-1.77 (m, 2H). |
| 471 | *** | ** | 497.3 | 1H NMR(400 MHz, DMSO-d6) δ 9.05 (d, J = 0.6 Hz, |
| | | | | 1H), 8.50 (s, 1H), 8.16 (d, J = 0.5 Hz, 1H), 7.65 (s, |
| | | | | 1H), 7.36-7.26 (m, 2H), 7.07-6.97 (m, 1H), 5.36 |
| | | | | (d, J = 5.8 Hz, 1H), 4.62-4.54 (m, 1H), 4.51-4.49 |
| | | | | (m, 2H), 3.98-3.96 (overlapping m, 1H), 3.96 |
| | | | | (overlapping s, 3H), 3.69-3.59 (m, 2H), 3.29-3.20 |
| | | | | (m, 2H), 2.13-2.04 (m, 2H), 1.91-1.78 (m, 2H), |
| | | | | 1.06 (d, J = 6.4 Hz, 3H). |
| 472 | *** | ** | 497.4 | 1H NMR(400 MHz, DMSO-d6) δ 9.05 (d, J = 0.6 Hz, |
| | | | | 1H), 8.50 (s, 1H), 8.16 (d, J = 0.4 Hz, 1H), 7.65 (s, |
| | | | | 1H), 7.36-7.25 (m, 2H), 7.07-6.97 (m, 1H), 5.37 |
| | | | | (d, J = 5.8 Hz, 1H), 4.61-4.54 (m, 1H), 4.54-4.45 |
| | | | | (m, 2H), 4.01-3.96 (overlapping m, 1H), 3.96 |
| | | | | (overlapping s, 3H), 3.71-3.56 (m, 2H), 3.30-3.18 |
| | | | | (m, 2H), 2.14-2.01 (m, 2H), 1.92-1.76 (m, 2H), |
| | | | | 1.05 (d, J = 5.5 Hz, 3H). |
| 473 | *** | ** | 496.5 | 1H NMR(400 MHz, DMSO-d6) δ 8.52-8.27 (m, |
| | | | | 1H), 7.33-7.23 (m, 2H), 7.21 (br s, 1H), 7.02-6.96 |
| | | | | (m, 1H), 6.96-6.90 (m, 1H), 4.66-4.55 (m, 2H), |
| | | | | 4.54-4.44 (m, 1H), 3.85-3.73 (m, 2H), 3.56-3.43 |
| | | | | (m, 2H), 3.13-3.01 (m, 2H), 2.96-2.88 (m, 1H), |
| | | | | 2.82 (d, 3H), 2.80-2.74 (m, 1H), 2.13 (s, 1.6H), 2.10 |
| | | | | (s, 1.4H), 1.92 (m, 2H), 1.68-1.52 (m, 2H). |
| 474 | **** | ** | 555.6 | 1H NMR(400 MHz, DMSO-d6) δ 8.41-8.32 (m, |
| | | | | 1H), 8.06 (s, 1H), 7.36-7.24 (m, 2H), 7.07-6.95 |
| | | | | (m, 1H), 4.73 (s, 1H), 4.59 (s, 1H), 4.55-4.41 (m, |
| | | | | 2H), 4.29-4.16 (m, 2H), 3.96-3.57 (m, 7H), 3.55- |
| | | | | 3.43 (m, 1H), 3.32-3.25 (overlapping, m, 2H), 3.07- |
| | | | | 2.84 (m, 3H), 2.81-2.72 (m, 1H), 2.12-2.01 (m, |
| | | | | 2H), 1.90-1.77 (m, 2H), 1.40 (t, J = 7.2 Hz, 3H). |
| 475 | **** | ** | 482.4 | 1H NMR(400 MHz, DMSO-d6) d 8.38-8.30 (m, |
| | | | | 1H), 7.35-7.21 (m, 2H), 7.05-6.95 (m, 1H), 6.94 |
| | | | | (d, J = 5.0 Hz, 1H), 6.76 (s, 2H), 4.67-4.55 (m, 2H), |
| | | | | 4.55-4.41 (m, 1H), 3.87-3.72 (m, 2H), 3.59-3.41 |
| | | | | (m, 2H), 3.15-2.99 (m, 2H), 2.91 (t, J = 5.7 Hz, 1H), |
| | | | | 2.78 (t, J = 5.8 Hz, 1H), 2.13 (s, 1.6H), 2.10 (s, 1.4H), |
| | | | | 2.02-1.84 (m, 2H), 1.72-1.54 (m, 2H). |
| 476 | ** | ** | 483.5 | 1H NMR(400 MHz, DMSO-d6) δ 7.81 (s, 1H), 7.75 |
| | | | | (s, 1H), 7.32-7.24 (m, 2H), 7.03-6.96 (m, 1H), |
| | | | | 4.61 (s, 0.90H), 4.57 (s, 1.11H, rotamers), 4.53-4.45 |
| | | | | (m, 1H), 4.07 (q, J = 7.2 Hz, 2H), 3.78 (q, J = 5.8 Hz, |
| | | | | 2H), 3.46-3.38 (m, 2H), 2.97 (t, J = 10.3 Hz, 2H), |
| | | | | 2.88 (t, J = 5.8 Hz, 1.15H), 2.75 (t, J = 5.8 Hz, 0.90H, |
| | | | | rotamers), 2.13 (s, 1.71H), 2.11 (s, 1.39H, rotamers), |
| | | | | 2.04-1.96 (m, 2H), 1.81-1.72 (m, 2H), 1.38 (t, J = |
| | | | | 7.3 Hz, 3H). |
| 477 | ** | ** | 494.5 | 1H NMR(400 MHz, DMSO-d6) δ 7.86-7.80 (m, |
| | | | | 1H), 7.59 (d, J = 7.7 Hz, 1H), 7.30-7.20 (m, 3H, |
| | | | | overlapped), 7.02-6.93 (m, 1H), 4.64 (s, 0.90H), |
| | | | | 4.59 (s, 1.07H, rotamers), 4.48-4.41 (m, 1H), 3.80 |
| | | | | (q, J = 5.7 Hz, 2H), 3.44-3.38 (m, 2H), 3.02-2.89 |
| | | | | (m, 3H, overlapped), 2.82-2.75 (m, 3H, overlapped), |
| | | | | 2.13 (s, 1.61H), 2.10 (s, 1.36H, rotamers), 1.88-1.79 |
| | | | | (m, 2H), 1.60-1.49 (m, 2H), 1.25 (t, J = 7.6 Hz, 3H) |
| 478 | *** | ** | 520.4 | 1H NMR(400 MHz, DMSO-d6) δ 8.39-8.34 (m, |
| | | | | 1H), 8.08-8.01 (m, 1H), 7.36-7.24 (m, 2H), 7.10- |
| | | | | 6.96 (overlapping m, 3H), 4.59-4.44 (m, 1H), 4.31- |
| | | | | 4.14 (m, 4H), 3.45-3.38 (m, 2H), 3.34 (overlapping |
| | | | | m, 2H), 3.02-2.87 (m, 4H), 2.14-2.00 (m, 2H), 1.91- |
| | | | | 1.75 (m, 2H), 1.46-1.33 (m, 3H); |
| 479 | ** | ** | 495.5 | 1H NMR(400 MHz, DMSO-d6) δ 8.88-8.79 (m, |
| | | | | 1H), 7.79-7.69 (m, 2H), 7.34-7.21 (m, 1H), 7.06- |
| | | | | 6.94 (m, 1H), 4.73-4.58 (m, 2H), 4.55-4.45 (m, |
| | | | | 1H), 3.90-3.75 (m, 2H), 3.54-3.40 (m, 2H), 3.13- |
| | | | | 3.01 (m, 2H), 2.99-2.89 (overlapped, m, 3H), 2.81 |
| | | | | (t, J = 5.8 Hz, 1H), 2.13 (s, 1.6H), 2.11 (s, 1.4H), 1.95- |
| | | | | 1.84 (m, 2H), 1.69-1.51 (m, 2H), 1.30 (t, J = 7.6 |
| | | | | Hz, 3H). |
| 480 | *** | ** | 437.4 | 1HNMR (400 MHz, DMSO-d6) δ 8.97 (s, 1H), 8.50 |
| | | | | (s, 1H), 8.45 (s, 1H), 8.16 (s, 1H), 7.36-7.26 (m, |
| | | | | 2H), 7.05-6.98 (m, 1H), 4.61-4.57 (m, 1H), 3.96 (s, |
| | | | | 3H), 3.74-3.65 (m, 2H), 3.33-3.26 (m, 2H), 2.54 (s, |
| | | | | 3H), 2.14-2.05 (m, 2H), 1.88-1.82 (m, 2H). |
| 481 | *** | ** | 574.5 | 1H NMR(400 MHz, DMSO-d6) δ 8.39-8.33 (m, |
| | | | | 1H), 8.10-8.02 (m, 1H), 7.36-7.24 (m, 2H), 7.06- |
| | | | | 6.96 (m, 1H), 4.87-4.57 (m, 2H), 4.57-4.46 (m, |
| | | | | 1H), 4.42-4.30 (m, 1H), 4.28-4.17 (m, 2H), 4.01- |
| | | | | 3.68 (m, 2H), 3.32-2.74 (m, 8H, overlapping), 2.59- |
| | | | | 2.52 (m, 1H, overlapping), 2.48-2.35 (m, 1H, |
| | | | | overlapping), 2.07 (m, 2H), 1.92-1.75 (m, 2H), 1.40 |
| | | | | (t, J = 7.3 Hz, 3H). NH proton could not be assigned. |
| 482 | *** | ** | 522.4 | 1H NMR(400 MHz, DMSO-d6) δ 9.15 (s, 1H), 8.51 |
| | | | | (s, 1H), 8.16 (s, 1H), 7.67 (s, 1H), 7.37-7.25 (m, |
| | | | | 2H), 7.06-6.97 (m, 1H), 5.72 (dd, J = 9.0, 6.1 Hz, |
| | | | | 1H), 4.64-4.51 (m, 1H), 4.00 (t, J = 8.9 Hz, 1H), |
| | | | | 3.95 (s, 3H), 3.73-3.60 (m, 3H), 3.30-3.22 |
| | | | | (overlapping m, 2H), 2.82 (s, 3H), 2.15-1.99 (m, |
| | | | | 2H), 1.91-1.73 (m, 2H). |
| 483 | *** | ** | 588.5 | 1H NMR(400 MHz, DMSO-d6) δ 8.40-8.34 (m, |
| | | | | 1H), 8.09-8.02 (m, 1H), 7.36-7.24 (m, 2H), 7.06- |
| | | | | 6.97 (m, 1H), 4.88-4.56 (m, 2H), 4.56-4.45 (m, |
| | | | | 1H), 4.29-4.17 (m, 2H), 4.05-3.72 (m, 3H), 3.46- |
| | | | | 3.36 (m, 1H, overlapping), 3.32-3.23 (m, 2H, |
| | | | | overlapping), 3.03-2.52 (m, 6H, overlapping), 2.47- |
| | | | | 2.18 (m, 4H, overlapping), 2.13-1.98 (m, 2H), |
| | | | | 1.91-1.74 (m, 2H), 1.40 (t, J = 7.3 Hz, 3H). |
| 484 | *** | ** | 529.5 | 1H NMR(400 MHz, DMSO-d6) δ 8.37 (s, 1H), 8.06 |
| | | | | (s, 1H), 7.40-7.23 (m, 2H), 7.09-6.95 (m, 1H), |
| | | | | 5.18 (br. s, 1H), 4.79 (s, 1H), 4.64 (s, 1H), 4.58-4.37 |
| | | | | (m, 2H), 4.22 (q, J = 6.6 Hz, 2H), 4.02-3.86 (m, |
| | | | | 2H), 3.85-3.69 (m, 1H), 3.65-3.43 (m, 2H), 3.41- |
| | | | | 3.23 (overlapping m, 2H), 2.96 (t, J = 10.2 Hz, 2H), |
| | | | | 2.92-2.86 (m, 1H), 2.86-2.74 (m, 1H), 2.16-1.96 |
| | | | | (m, 2H), 1.93-1.70 (m, 2H), 1.40 (t, J = 7.2 Hz, 3H). |
| 485 | *** | ** | 518.4 | 1H NMR(400 MHz, DMSO-d6) δ 8.32 (s, 1H), 8.01 |
| | | | | (s, 1H), 7.31-7.21 (m, 2H), 7.01-6.92 (m, 1H), |
| | | | | 4.53-4.43 (m, 1H), 4.31 (q, J = 15.8 Hz, 2H), 4.19 |
| | | | | (q, J = 7.2 Hz, 2H), 3.80 (d, J = 1.5 Hz, 1H), 3.55- |
| | | | | 3.42 (m, 2H), 3.29-3.24 (overlapping m, 2H), 2.98- |
| | | | | 2.84 (m, 4H), 2.82 (d, J = 1.4 Hz, 3H), 2.10-1.96 |
| | | | | (m, 2H), 1.87-1.72 (m, 2H), 1.37 (t, J = 7.3 Hz, 3H) |
| 486 | *** | ** | 483.3 | 1H NMR(400 MHz, DMSO-d6) δ 8.33 (s, 1H), 8.06 |
| | | | | (s, 1H), 7.38-7.22 (m, 2H), 7.08-6.94 (m, 1H), |
| | | | | 4.60-4.46 (m, 3H), 4.34-4.30 (m, 1H), 4.24-4.13 |
| | | | | (m, 2H), 3.92 (s, 3H), 3.32-3.29 (overlapping m, |
| | | | | 2H), 3.03-2.86 (m, 4H), 2.12-1.98 (m, 2H), 1.90- |
| | | | | 1.76 (m, 2H). |
| 487 | **** | ** | 437.4 | 1HNMR (400 MHz, DMSO-d6) δ 8.51 (s, 1H), 8.40 |
| | | | | (d, J = 5.7 Hz, 1H), 8.19 (s, 1H), 7.46 (d, J = 5.7 Hz, |
| | | | | 1H), 7.35-7.26 (m, 2H), 7.05-6.99 (m, 1H), 4.59- |
| | | | | 4.56 (m, 1H), 3.96 (s, 3H), 3.68-3.59 (m, 2H), 3.33- |
| | | | | 3.22 (m, 2H), 2.86 (s, 3H), 2.11-2.03 (m, 2H), 1.86- |
| | | | | 1.83 (m, 2H). |
| 488 | *** | ** | 497.4 | 1H NMR(400 MHz, DMSO-d6) δ 8.33 (s, 1H), 8.06 |
| | | | | (s, 1H), 7.34-7.25 (m, 2H), 7.04-6.97 (m, 1H), |
| | | | | 5.18 (d, J = 17.3 Hz, 1H), 4.57-4.46 (m, 1H), 4.17 |
| | | | | (d, J = 17.4 Hz, 1H), 4.13 (s, 2H), 4.07 (dd, J = 11.9, |
| | | | | 3.8 Hz, 1H), 3.96-3.88 (m, 4H), 3.85 (dd, J = 11.9, |
| | | | | 3.7 Hz, 1H), 3.32-3.28 (submerged m, 2H), 3.09- |
| | | | | 2.91 (m, 3H), 2.83 (dd, J = 16.6, 3.6 Hz, 1H), 2.10- |
| | | | | 2.04 (m, 2H), 1.90-1.76 (m, 2H). |
| 489 | *** | ** | 459.4 | 1H NMR(400 MHz, DMSO-d6) δ 7.36-7.26 (m, |
| | | | | 2H), 7.05-6.98 (m, 1H), 4.65 (dd, J = 9.8, 5.0 Hz, |
| | | | | 1H), 4.58-4.47 (m, 3H, overlapped), 3.79-3.68 (m, |
| | | | | 2H), 3.63-3.53 (m, 2H), 3.50-3.40 (m, 1H), 3.40- |
| | | | | 3.34 (m, 1H), 3.17-3.08 (m, 1H), 3.06-2.97 (m, |
| | | | | 1H), 2.84 (t, J = 5.8 Hz, 1.1H), 2.71 (t, J = 5.6 Hz, |
| | | | | 0.83H, rotamers), 2.12-2.03 (m, 6H, overlapped), |
| | | | | 1.87-1.74 (m, 2H), 1.61-1.51 (m, 1H), 1.20-1.12 |
| | | | | (m, 1H), 0.99-0.90 (m, 1H). |
| 490 | *** | ** | 499.4 | 1H NMR(400 MHz, DMSO-d6) δ 8.32 (s, 1H), 8.08- |
| | | | | 8.03 (m, 1H), 7.37-7.22 (m, 2H), 7.06-6.95 (m, |
| | | | | 1H), 5.08 (d, J = 18.1 Hz, 0.65H), 5.02 (t, J = 5.5 Hz, |
| | | | | 0.65H), 4.98-4.91 (m, 0.35H), 4.84 (t, J = 5.6 Hz, |
| | | | | 0.35H), 4.72 (d, J = 17.1 Hz, 0.35H), 4.56-4.47 (m, |
| | | | | 1.35H), 4.36-4.27 (m, 0.65H), 4.01 (d, J = 18.1 Hz, |
| | | | | 0.65H), 3.92 (s, 3H), 3.43-3.34 (m, 2H), 3.30-3.27 |
| | | | | (submerged m, 2H), 3.26-3.10 (m, 1H), 3.04-2.87 |
| | | | | (m, 2H), 2.87-2.68 (m, 1H), 2.17-2.11 (m, 3H), |
| | | | | 2.10-1.99 (m, 2H), 1.91-1.75 (m, 2H). |
| 491 | **** | ** | 554.5 | 1H NMR(400 MHz, DMSO-d6) δ 8.39-8.35 (m, |
| | | | | 1H), 8.06 (s, 1H), 7.35-7.25 (m, 2H), 7.06-6.96 |
| | | | | (m, 1H), 4.96-4.43 (m, 2H), 4.30-4.15 (m, 2H), |
| | | | | 4.01-3.56 (m, 5H), 3.39-3.25 (m, 3H, |
| | | | | overlapping), 3.04-2.86 (m, 3H), 2.84-2.70 (m, |
| | | | | 3H), 2.54-2.45 (m, 2H, overlapping), 2.35-2.24 |
| | | | | (m, 1H), 2.14-1.99 (m, 2H), 1.91-1.75 (m, 2H), |
| | | | | 1.40 (t, J = 7.2 Hz, 3H). |
| 492 | *** | ** | 568.5 | 1H NMR(400 MHz, DMSO-d6) δ 8.37 (s, 1H), 8.06 |
| | | | | (s, 1H), 7.36-7.24 (m, 2H), 7.06-6.96 (m, 1H), |
| | | | | 5.17-4.41 (m, 2H), 4.35-3.99 (m, 3H), 3.91-3.65 |
| | | | | (m, 3H), 3.63-3.39 (m, 2H), 3.31-3.22 (m, 2H, |
| | | | | overlapping), 3.06-2.69 (m, 5H), 2.30-1.99 (m, |
| | | | | 6H), 1.92-1.73 (m, 2H), 1.41 (t, J = 7.2 Hz, 3H). 2H |
| | | | | overlapping with the residual DMSO peak. |
| 493 | ** | ** | 495.5 | 1H NMR(400 MHz, DMSO-d6) δ 8.88 (s, 1H), 8.57- |
| | | | | 8.55 (m, 1H), 7.32-7.21 (m, 2H), 7.02-6.94 (m, |
| | | | | 1H), 4.67 (s, 0.89H), 4.62 (s, 1.08H, rotamers), 4.52- |
| | | | | 4.44 (m, 1H), 3.81 (dd, J = 10.0, 5.7 Hz, 2H), 3.46- |
| | | | | 3.36 (m, 2H), 3.07-2.98 (m, 2H), 2.94 (t, J = 5.8 Hz, |
| | | | | 1H), 2.89-2.78 (m, 3H, overlapped), 2.14 (s, 1.62H), |
| | | | | 2.11 (s, 1.41H, rotamers), 1.89-1.82 (m, 2H), 1.62- |
| | | | | 1.52 (m, 2H), 1.28 (t, J = 7.6 Hz, 3H). |
| 494 | *** | ** | 471.4 | 1H NMR(400 MHz, DMSO-d6) δ 7.35-7.26 (m, |
| | | | | 2H), 7.05-6.98 (m, 1H), 6.57-6.52 (m, 1H), 4.69- |
| | | | | 4.49 (m, 5H, overlapped), 3.81-3.73 (m, 4H, |
| | | | | overlapped), 3.45-3.37 (m, 2H), 3.04 (t, J = 10.1 Hz, |
| | | | | 2H), 2.87 (t, J = 5.7 Hz, 1.16H), 2.80 (t, J = 6.5 Hz, |
| | | | | 2H), 2.74 (t, J = 5.7 Hz, 0.82H, rotamers), 2.12 (s, |
| | | | | 1.62H), 2.11 (s, 1.37H, rotamers), 2.09-2.03 (m, |
| | | | | 2H), 1.86-1.76 (m, 2H), |
| Cmpd No. | | | | |
| 495 | ** | * | 433 | 1H NMR (400 MHz, MeOD) δ 7.91 (s, 1H), 7.77 (d, J = |
| | | | | 0.6 Hz, 1H), 7.44 (dt, J = 16.2, 7.5 Hz, 5H), 5.28-5.24 |
| | | | | (m, 1H), 4.08 (s, 2H), 3.94 (s, 3H), 3.89 (s, 2H), 3.56 (dd, |
| | | | | J = 10.1, 5.2 Hz, 1H), 3.53-3.47 (m, 1H), 3.24 (dd, J = |
| | | | | 17.0, 7.9 Hz, 4H), 3.08-2.96 (m, 2H), 2.21-2.10 (m, |
| | | | | 1H), 2.07-1.99 (m, 4H). |
| 496 | ** | ** | 554.6 | 1H NMR (400 MHz, DMSO-d6) δ 7.36-7.25 (m, 2H), |
| | | | | 7.07-6.96 (m, 1H), 4.64-4.56 (m, 2H), 4.56-4.47 (m, |
| | | | | 1H), 3.81-3.72 (m, 2H), 3.31-3.23 (m, 2H), 3.22- |
| | | | | 3.13 (m, 2H), 3.05-2.93 (overlapped, m, 4H), 2.91-2.80 |
| | | | | (m, 2H), 2.79-2.70 (m, 1H), 2.48-2.39 (m, 2H), 2.11 |
| | | | | (s, 1.5H), 2.10 (s, 1.5H), 2.09-2.01 (m, 2H), 1.90-1.73 |
| | | | | (overlapped, m, 4H), 1.67 (m, 2H). |
| 497 | *** | ** | 476.2 | 1H NMR (400 MHz, DMSO-d6): δ 8.33 (s, 1H), 8.06 (d, |
| | | | | J = 1.9 Hz, 1H), 7.75 (dd, J = 8.2, 3.2 Hz, 1H), 7.59- |
| | | | | 7.52 (m, 1H), 7.40 (dd, J = 9.4, 4.3 Hz, 1H), 4.81-4.73 |
| | | | | (m, 1H), 4.62 (d, J = 15.4 Hz, 2H), 3.92 (d, J = 1.6 Hz, |
| | | | | 3H), 3.82-3.76 (m, 2H), 3.33-3.26 (m, 2H), 3.09- |
| | | | | 3.01 (m, 2H), 2.90 (t, J = 5.6 Hz, 1H), 2.77 (t, J = 5.7 Hz, |
| | | | | 1H), 2.11 (t, J = 10.5 Hz, 5H), 1.94-1.83 (m, 2H). |
| 498 | ** | ** | 464.4 | 1H NMR (400 MHz, DMSO-d6): δ 8.32 (s, 1H), 8.05 (d, |
| | | | | J = 1.7 Hz, 1H), 7.91 (d, J = 2.9 Hz, 1H), 7.46 (dd, J = |
| | | | | 9.0, 3.0 Hz, 1H), 6.77 (d, J = 8.9 Hz, 1H), 4.62 (d, J = |
| | | | | 16.2 Hz, 2H), 4.51-4.43 (m, 1H), 3.92 (d, J = 1.7 Hz, |
| | | | | 3H), 3.79 (s, 5H), 3.31-3.27 (m, 2H), 2.99-2.87 (m, |
| | | | | 3H), 2.77 (t, J = 5.5 Hz, 1H), 2.09 (dd, J = 23.6, 7.5 Hz, |
| | | | | 5H), 1.86-1.76 (m, 2H). |
| 499 | *** | * | 458.2 | 1H NMR (400 MHz, DMSO-d6): δ 8.33 (s, 1H), 8.06 (d, |
| | | | | J = 1.8 Hz, 1H), 7.73 (dd, J = 7.7, 1.5 Hz, 1H), 7.68- |
| | | | | 7.62 (m, 1H), 7.36 (d, J = 8.6 Hz, 1H), 7.09 (t, J = 7.5 Hz, |
| | | | | 1H), 4.81 (s, 1H), 4.62 (d, J = 15.5 Hz, 2H), 3.92 (s, 3H), |
| | | | | 3.79 (d, J = 4.2 Hz, 2H), 3.31-3.27 (m, 2H), 3.10-3.02 |
| | | | | (m, 2H), 2.91 (t, J = 5.5 Hz, 1H), 2.77 (t, J = 5.5 Hz, 1H), |
| | | | | 2.12 (d, J = 4.8 Hz, 5H), 1.88 (dd, J = 10.7, 6.3 Hz, 2H). |
| 500 | ** | ** | 476.2 | 1H NMR (400 MHz, DMSO-d6): δ 8.33 (s, 1H), 8.06 (d, |
| | | | | J = 2.1 Hz, 1H), 7.86 (dd, J = 11.3, 2.0 Hz, 1H), 7.67 (d, |
| | | | | J = 8.6 Hz, 1H), 7.48 (t, J = 8.6 Hz, 1H), 4.85-4.78 (m, |
| | | | | 1H), 4.62 (d, J = 16.0 Hz, 2H), 3.92 (d, J = 1.6 Hz, 3H), |
| | | | | 3.82-3.76 (m, 2H), 3.32-3.27 (m, 2H), 3.01 (t, J = 9.8 |
| | | | | Hz, 2H), 2.90 (t, J = 5.7 Hz, 1H), 2.77 (t, J = 5.7 Hz, 1H), |
| | | | | 2.12 (d, J = 4.8 Hz, 5H), 1.94-1.83 (m, 2H). |
| 501 | *** | *** | 519.2 | 1H NMR (400 MHz, DMSO-d6): δ 8.34 (s, 1H), 8.06 (d, |
| | | | | J = 2.1 Hz, 1H), 7.73-7.67 (m, 1H), 7.55-7.45 (m, 2H), |
| | | | | 4.82-4.75 (m, 1H), 4.63 (d, J = 16.1 Hz, 2H), 3.92 (d, |
| | | | | J = 1.6 Hz, 3H), 3.83-3.76 (m, 2H), 3.31-3.28 (m, 2H), |
| | | | | 3.01 (t, J = 9.7 Hz, 2H), 2.91 (t, J = 5.7 Hz, 1H), 2.77 (t, |
| | | | | J = 5.7 Hz, 1H), 2.12 (d, J = 4.8 Hz, 5H), 1.95-1.84 (m, 2H). |
| 502 | *** | ** | 481.1 | 1H NMR (400 MHz, DMSO-d6): δ 8.31 (s, 1H), 8.05 (d, |
| | | | | J = 1.7 Hz, 1H), 7.10-7.04 (m, 1H), 6.95 (dd, J = 13.2, |
| | | | | 2.9 Hz, 1H), 6.80-6.75 (m, 1H), 4.62 (d, J = 16.1 Hz, |
| | | | | 2H), 4.52-4.43 (m, 1H), 3.92 (d, J = 1.6 Hz, 3H), 3.83- |
| | | | | 3.75 (m, 5H), 3.33-3.26 (m, 2H), 2.93 (dt, J = 11.4, 8.4 |
| | | | | Hz, 3H), 2.76 (t, J = 5.7 Hz, 1H), 2.12 (d, J = 5.1 Hz, 3H), |
| | | | | 2.09-2.01 (m, 2H), 1.84-1.74 (m, 2H). |
| 503 | ** | ** | 515.1 | 1H NMR (400 MHz, DMSO-d6): δ 8.31 (s, 1H), 8.05 (d, |
| | | | | J = 1.6 Hz, 1H), 7.42 (d, J = 8.7 Hz, 1H), 7.19 (d, J = 12.9 |
| | | | | Hz, 1H), 4.62 (d, J = 15.8 Hz, 2H), 4.47 (d, J = 3.8 Hz, |
| | | | | 1H), 3.92 (d, J = 1.5 Hz, 3H), 3.85-3.75 (m, 4H), 3.31- |
| | | | | 3.26 (m, 2H), 2.93 (dd, J = 22.4, 8.3 Hz, 3H), 2.77 (t, J = |
| | | | | 5.6 Hz, 1H), 2.14-2.09 (m, 3H), 2.05 (d, J = 19.1 Hz, |
| | | | | 2H), 1.87-1.75 (m, 2H). |
| 504 | *** | ** | 495.1 | 1H NMR (400 MHz, DMSO-d6): δ 8.31 (s, 1H), 8.05 (d, |
| | | | | J = 1.7 Hz, 1H), 7.15 (t, J = 9.4 Hz, 1H), 6.86 (dd, J = |
| | | | | 13.1, 2.9 Hz, 1H), 6.70-6.65 (m, 1H), 4.62 (d, J = 16.0 |
| | | | | Hz, 2H), 4.41-4.31 (m, 1H), 4.01-3.90 (m, 5H), 3.79 |
| | | | | (dd, J = 9.9, 5.7 Hz, 2H), 3.32-3.23 (m, 2H), 2.98-2.86 |
| | | | | (m, 3H), 2.76 (t, J = 5.6 Hz, 1H), 2.12 (d, J = 5.0 Hz, 3H), |
| | | | | 2.02 (s, 2H), 1.87-1.74 (m, 3H), 1.31(t, J = 6.8 Hz, 3H |
| 505 | *** | ** | 468.3 | 1H NMR (400 MHz, DMSO-d6): δ 8.27 (s, 1H), 8.03 (s, |
| | | | | 1H), 7.48 (dd, J = 15.4, 8.4 Hz, 1H), 7.21 (td, J = 10.2, |
| | | | | 2.5 Hz, 1H), 7.11-7.03 (m, 1H), 4.61 (d, J = 15.5 Hz, |
| | | | | 2H), 3.90 (d, J = 1.5 Hz, 3H), 3.83-3.73 (m, 2H), 3.58 |
| | | | | (s, 2H), 3.05 (s, 4H), 2.88 (t, J = 5.7 Hz, 1H), 2.79-2.71 |
| | | | | (m, 1H), 2.57 (s, 4H), 2.11 (d, J = 5.9 Hz, 3H). |
| 506 | *** | ** | 483.9 | 1H-NMR (400 MHz, DMSO-d6) δ 8.29 (s, 1H), 8.06 (d, |
| | | | | J = 1.8 Hz, 1H), 7.49 (dd, J = 8.8, 5.2 Hz, 1H), 7.36 (dd, |
| | | | | J = 9.6, 3.1 Hz, 1H), 7.17 (td, J = 8.5, 3.1 Hz, 1H), 4.62 (d, |
| | | | | J = 15.8 Hz, 2H), 3.92 (s, 3H), 3.79 (d, J = 4.3 Hz, 2H), |
| | | | | 3.66 (s, 2H), 3.09 (s, 4H), 2.90 (t, J = 5.6 Hz, 1H), 2.76 (t, |
| | | | | J = 5.6 Hz, 1H), 2.66 (d, J = 10.8 Hz, 4H), 2.12 (d, J = 5.6 |
| | | | | Hz, 3H). |
| 507 | *** | ** | 466.2 | 1H NMR (400 MHz, DMSO-d6) δ 8.29 (s, 1H), 8.05 (d, |
| | | | | J = 1.8 Hz, 1H), 7.53 (dd, J = 7.5, 1.7 Hz, 1H), 7.44 (dd, |
| | | | | J = 7.7, 1.4 Hz, 1H), 7.31 (dtd, J = 16.9, 7.4, 1.6 Hz, 2H), |
| | | | | 4.61 (d, J = 15.7 Hz, 2H), 3.91 (d, J = 1.7 Hz, 3H), 3.84- |
| | | | | 3.74 (m, 2H), 3.66 (s, 2H), 3.07 (s, 4H), 2.89 (t, J = 5.6 |
| | | | | Hz, 1H), 2.76 (t, J = 5.7 Hz, 1H), 2.63 (s, 4H), 2.12 (d, J = |
| | | | | 5.6 Hz, 3H). |
| 508 | *** | ** | 509.1 | 1H NMR (400 MHz, DMSO-d6) δ 8.29 (s, 1H), 8.06 (d, |
| | | | | J = 1.9 Hz, 1H), 7.61 (dd, J = 7.9, 0.9 Hz, 1H), 7.52 (dd, |
| | | | | J = 7.6, 1.4 Hz, 1H), 7.38 (dd, J = 10.7, 4.2 Hz, 1H), 7.21 |
| | | | | (td, J = 7.8, 1.6 Hz, 1H), 4.62 (d, J = 15.7 Hz, 2H), 3.92 |
| | | | | (d, J = 1.7 Hz, 3H), 3.81-3.76 (m, 2H), 3.64 (s, 2H), |
| | | | | 3.08 (s, 4H), 2.89 (t, J = 5.6 Hz, 1H), 2.76 (t, J = 5.6 Hz, |
| | | | | 1H), 2.63 (s, 4H), 2.12 (d, J = 5.5 Hz, 3H). |
| 509 | * | ** | 462.2 | 1H NMR (400 MHz, DMSO-d6) δ 8.28 (s, 1H), 8.04 (s, |
| | | | | 1H), 7.34 (d, J = 7.2 Hz, 1H), 7.23 (t, J = 7.5 Hz, 1H), |
| | | | | 6.98 (d, J = 8.2 Hz, 1H), 6.93 (t, J = 7.3 Hz, 1H), 4.61 (d, |
| | | | | J = 15.5 Hz, 2H), 3.91 (s, 3H), 3.79 (s, 5H), 3.55 (s, 2H), |
| | | | | 3.06 (s, 4H), 2.89 (t, J = 5.6 Hz, 1H), 2.76 (t, J = 5.5 Hz, |
| | | | | 1H), 2.58 (s, 4H), 2.16-2.08 (m, 3H). |
| 510 | ** | ** | 446.2 | 1H NMR (400 MHz, DMSO-d6) δ 8.29 (s, 1H), 8.13 (s, |
| | | | | 0.5H), 8.07 (s, 1H), 7.27 (s, 1H), 7.17 (s, 3H), 4.61 (d, J = |
| | | | | 15.4 Hz, 2H), 3.92 (s, 3H), 3.78 (d, J = 4.5 Hz, 2H), 3.55 |
| | | | | (s, 2H), 3.05 (s, 4H), 2.89 (t, J = 5.4 Hz, 1H), 2.75 (d, J = |
| | | | | 5.3 Hz, 1H), 2.54 (s, 2H), 2.35 (s, 3H), 2.11 (d, J = 5.4 |
| | | | | Hz, 3H). |
| 511 | * | * | 433 | 1H NMR (400 MHz, DMSO-d6) δ 8.50 (d, J = 4.0 Hz, |
| | | | | 1H), 8.28 (s, 1H), 8.04 (s, 1H), 7.77 (td, J = 7.7, 1.7 Hz, |
| | | | | 1H), 7.47 (d, J = 7.8 Hz, 1H), 7.26 (dd, J = 6.5, 5.0 Hz, |
| | | | | 1H), 4.61 (d, J = 15.7 Hz, 2H), 3.91 (d, J = 1.6 Hz, 3H), |
| | | | | 3.83-3.74 (m, 2H), 3.68 (s, 2H), 3.08 (s, 4H), 2.89 (t, J = |
| | | | | 5.6 Hz, 1H), 2.79-2.72 (m, 1H), 2.61 (s, 4H), 2.12 (d, |
| | | | | J = 5.8 Hz, 3H). |
| 512 | ** | ** | 457 | 1H NMR (400 MHz, DMSO-d6): δ 8.27 (s, 1H), 8.04 (s, |
| | | | | 1H), 7.79-7.67 (m, 3H), 7.56 (t, J = 7.7 Hz, 1H), 4.61 |
| | | | | (d, J = 15.6 Hz, 2H), 3.90 (s, 3H), 3.83-3.71 (m, 2H), |
| | | | | 3.62 (s, 2H), 3.07 (bs, 4H), 2.89 (t, J = 5.6 Hz, 1H), 2.80- |
| | | | | 2.72 (m, 1H), 2.55 (bs, 4H), 2.12 (d, J = 6.0 Hz, 3H). |
| 513 | ** | ** | 512.1 | 1H NMR (400 MHz, DMSO-d6): δ 8.32 (s, 1H), 8.28 (d, |
| | | | | J = 2.5 Hz, 1H), 8.05 (d, J = 2.0 Hz, 1H), 7.90 (dd, J = |
| | | | | 8.8, 2.6 Hz, 1H), 6.83 (d, J = 8.8 Hz, 1H), 5.13 (dd, J = |
| | | | | 8.0, 4.0 Hz, 1H), 4.62 (d, J = 16.4 Hz, 2H), 3.92 (s, 3H), |
| | | | | 3.82-3.76 (m, 2H), 3.34 (s, 2H), 3.01-2.93 (m, 2H), |
| | | | | 2.90 (t, J = 5.7 Hz, 1H), 2.77 (t, J = 5.6 Hz, 1H), 2.12 (d, |
| | | | | J = 5.0 Hz, 5H), 1.92-1.81 (m, 2H). |
| 514 | ** | ** | 438 | 1H NMR (400 MHz, DMSO-d6): δ 8.26 (s, 1H), 8.03 (s, |
| | | | | 1H), 7.49 (dd, J = 4.9, 3.0 Hz, 1H), 7.34 (d, J = 1.9 Hz, |
| | | | | 1H), 7.07 (dd, J = 4.9, 0.9 Hz, 1H), 4.61 (d, J = 15.5 Hz, |
| | | | | 2H), 3.90 (s, 3H), 3.82-3.75 (m, 2H), 3.56 (s, 2H), 3.05 |
| | | | | (s, 4H), 2.89 (t, J = 5.7 Hz, 1H), 2.75 (t, J = 5.7 Hz, 1H), |
| | | | | 2.54 (s, 4H), 2.11 (d, J = 5.9 Hz, 3H). |
| 515 | ** | ** | 438 | 1H NMR (400 MHz, DMSO-d6): δ 8.26 (s, 1H), 8.03 (s, |
| | | | | 1H), 7.44 (dd, J = 4.9, 1.3 Hz, 1H), 6.98 (dd, J = 8.3, 3.4 |
| | | | | Hz, 2H), 4.61 (d, J = 15.5 Hz, 2H), 3.90 (s, 3H), 3.81- |
| | | | | 3.75 (m, 4H), 3.06 (s, 4H), 2.89 (t, J = 5.6 Hz, 1H), 2.76 |
| | | | | (t, J = 5.6 Hz, 1H), 2.59 (s, 4H), 2.12 (d, J = 5.9 Hz, 3H). |
| 516 | ** | * | 438 | 1H NMR (400 MHz, DMSO-d6): δ 9.05 (d, J = 1.9 Hz, |
| | | | | 1H), 8.26 (s, 1H), 8.03 (s, 1H), 7.54 (d, J = 1.8 Hz, 1H), |
| | | | | 4.61 (d, J = 15.5 Hz, 3H), 3.90 (s, 4H), 3.81-3.72 (m, |
| | | | | 6H), 3.06 (s, 5H), 2.89 (t, J = 5.6 Hz, 2H), 2.76 (t, J = 5.5 |
| | | | | Hz, 1H), 2.62 (s, 6H), 2.11 (d, J = 5.9 Hz, 4H). |
| 517 | * | * | 433.2 | 1H NMR (400 MHz, DMSO-d6): δ 8.52 (s, 1H), 8.49- |
| | | | | 8.45 (m, 1H), 8.27 (s, 1H), 8.04 (s, 1H), 7.74 (d, J = 7.8 |
| | | | | Hz, 1H), 7.37 (dd, J = 7.6, 5.0 Hz, 1H), 4.61 (d, J = 15.5 |
| | | | | Hz, 2H), 3.90 (s, 3H), 3.78 (d, J = 4.3 Hz, 2H), 3.59 (s, |
| | | | | 2H), 3.06 (s, 4H), 2.89 (s, 1H), 2.75 (s, 1H), 2.56 (s, 4H), |
| | | | | 2.11 (d, J = 5.9 Hz, 3H). |
| 518 | ** | ** | 433.2 | 1H NMR (400 MHz, DMSO-d6): δ 8.51 (d, J = 5.8 Hz, |
| | | | | 2H), 8.27 (s, 1H), 8.04 (s, 1H), 7.35 (d, J = 5.8 Hz, 2H), |
| | | | | 4.61 (d, J = 15.6 Hz, 2H), 3.90 (s, 3H), 3.82-3.75 (m, |
| | | | | 2H), 3.60 (s, 2H), 3.08 (s, 4H), 2.89 (t, J = 5.6 Hz, 1H), |
| | | | | 2.76 (t, J = 5.7 Hz, 1H), 2.57 (s, 4H), 2.12 (d, J = 6.0 Hz, 3H). |
| 519 | *** | ** | 543.1 | 1H NMR (400 MHz, DMSO-d6) δ 8.37 (s, 1H), 8.06 (s, |
| | | | | 1H), 7.38-7.22 (m, 2H), 7.00 (t, J = 7.2 Hz, 1H), 5.45 (s, |
| | | | | 1H), 5.14 (br. s, 1H), 4.83-4.42 (m, 3H), 4.33-4.09 (m, |
| | | | | 3H), 3.88 (br. s, 1H), 3.62-3.40 (m, 2H), 3.32-3.25 |
| | | | | (overlapping m, 2H), 3.04-2.71 (m, 4H), 2.17-2.00 (m, |
| | | | | 2H), 1.94-1.75 (m, 2H), 1.41 (t, J = 7.1 Hz, 3H), 1.29 (s, 3H) |
| 520 | ** | ** | 517.4 | 1H NMR (400 MHz, DMSO-d6) δ 7.40-7.22 (m, 2H), |
| | | | | 7.01 (t, J = 8.5 Hz, 1H), 4.75-4.63 (m, 1H), 4.64-4.41 |
| | | | | (m, 3H), 4.20 (s, 2H), 3.80 (d, J = 4.0 Hz, 1H), 3.69 (d, |
| | | | | J = 4.4 Hz, 1H), 3.47 (s, 1H), 3.40-3.11 (overlapping m, |
| | | | | 4H), 3.15-2.70 (m, 5H), 2.16-1.71 (m, 9H), 1.61- |
| | | | | 1.33 (m, 4H); |
| 521 | *** | ** | 557.5 | 1H NMR (400 MHz, DMSO-d6) § 8.39-8.34 (m, 1H), |
| | | | | 8.09-8.01 (m, 1H), 7.37-7.23 (m, 2H), 7.06-6.94 (m, |
| | | | | 1H), 5.00-4.57 (m, 4H), 4.57-4.43 (m, 1H), 4.35- |
| | | | | 4.14 (m, 3H), 4.06-3.78 (m, 2H), 3.31-3.25 |
| | | | | (overlapping m, 2H), 3.04-2.70 (m, 4H), 2.17-1.99 (m, |
| | | | | 2H), 1.92-1.73 (m, 2H), 1.47-1.33 (m, 3H), 1.29- |
| | | | | 1.14 (m, 3H), 1.12-0.96 (m, 3H) |
| 522 | ** | ** | 448.4 | 1H-NMR (400 MHz, DMSO-d6) δ 8.26 (s, 1H), 8.05 (s, |
| | | | | 1H), 7.65 (dd, J = 6.7, 1.8 Hz, 1H), 7.40 (ddd, J = 8.9, |
| | | | | 6.6, 2.0 Hz, 1H), 6.38 (dd, J = 9.1, 0.6 Hz, 1H), 6.20 (td, |
| | | | | J = 6.7, 1.3 Hz, 1H), 4.65-4.53 (m, 2H), 3.92 (s, 1.5H), |
| | | | | 3.91 (s, 1.5H), 3.86 (d, J = 7.2 Hz, 2H), 3.77 (dd, J = 10.2, |
| | | | | 5.7 Hz, 2H), 3.40 (d, J = 10.6 Hz, 2H), 2.87 (t, J = 5.7 Hz, |
| | | | | 1H), 2.74 (t, J = 5.7 Hz, 1H), 2.59 (t, J = 12.1 Hz, 2H), |
| | | | | 2.12 (s, 1.5H), 2.10 (s, 1.5H), 1.98-1.81 (m, 1H), 1.63- |
| | | | | 1.53 (m, 2H), 1.52-1.35 (m, 2H). Rotamers present |
| 523 | * | * | 439.2 | 1H NMR (400 MHz, DMSO-d6): δ 8.72(s, 1H) 8.39 (s, |
| | | | | 1H), 8.07 (d, J = 2.8 Hz, 2H), 7.17 (d, J = 4 Hz, 1H), 4.64 (d, |
| | | | | J = 15.6 Hz, 2H), 3.91 (s, 3H), 3.79(d, J = 3.2 Hz, 6H), |
| | | | | 3.09 (s, 4H), 2.91 (t, J = 5.6 Hz, 1H), 2.78 (t, J = 5.6 Hz, |
| | | | | 1H), 2.12(d, J = 5.9 Hz, 3H). |
| 524 | * | * | 422.1 | 1H NMR (400 MHz, DMSO-d6): δ 8.26 (s, 1H), 8.02 (s, |
| | | | | 1H), 7.54 (s, 1H), 6.16 (d, J = 1.9 Hz, 1H), 4.61 (d, J = |
| | | | | 15.3 Hz, 2H), 3.92 (d, J = 16.1 Hz, 3H), 3.81-3.74 (m, |
| | | | | 2H), 3.53 (d, J = 19.9 Hz, 2H), 3.04 (s, 4H), 2.89 (t, J = |
| | | | | 5.6 Hz, 1H), 2.75 (t, J = 5.6 Hz, 1H), 2.55 (s, 4H), 2.11 |
| | | | | (d, J = 5.9 Hz, 2H). |
| 525 | * | * | 436.3 | 1H NMR (400 MHz, DMSO-d6): δ 8.28 (s, 1H), 8.05 (s, |
| | | | | 1H), 7.31 (d, J = 1.7 Hz, 1H), 6.16 (d, J = 1.7 Hz, 1H), |
| | | | | 4.61 (d, J = 15.7 Hz, 2H), 3.91 (d, J = 1.6 Hz, 3H), 3.83- |
| | | | | 3.75 (m, 5H), 3.62 (d, J = 12.1 Hz, 2H), 3.04 (s, 4H), 2.88 |
| | | | | (t, J = 5.7 Hz, 1H), 2.75 (t, J = 5.7 Hz, 1H), 2.55 (s, 4H), |
| | | | | 2.11 (d, J = 5.4 Hz, 3H). |
| 526 | * | *** | 436.3 | 1H NMR (400 MHz, DMSO-d6): δ 8.25 (s, 1H), 8.02 (s, |
| | | | | 1H), 7.58 (s, 1H), 7.31 (s, 1H), 4.60 (d, J = 15.5 Hz, 2H), |
| | | | | 3.90 (d, J = 1.6 Hz, 3H), 3.82-3.76 (m, 5H), 3.39 (s, |
| | | | | 2H), 3.03 (s, 4H), 2.88 (t, J = 5.6 Hz, 1H), 2.75 (t, J = 5.3 |
| | | | | Hz, 1H), 2.60-2.48 (m, 4H), 2.11 (d, J = 6.1 Hz, 3H). |
| 527 | *** | ** | 436.3 | 1H NMR (400 MHz, DMSO-d6): δ 8.30 (s, 1H), 8.07 (d, |
| | | | | J = 1.8 Hz, 1H), 7.89 (d, J = 8.1 Hz, 1H), 7.69 (d, J = 4.2 |
| | | | | Hz, 2H), 7.55 (td, J = 8.3, 4.1 Hz, 1H), 4.63 (d, J = 16.0 |
| | | | | Hz, 2H), 3.93 (d, J = 1.6 Hz, 3H), 3.85 (s, 2H), 3.83- |
| | | | | 3.76 (m, 2H), 3.01 (s, 4H), 2.91 (t, J = 5.6 Hz, 1H), 2.78 |
| | | | | (t, J = 5.6 Hz, 1H), 2.54 (d, J = 4.9 Hz, 4H), 2.14 (d, J = |
| | | | | 5.4 Hz, 3H). |
| 528 | * | * | 436.3 | 1H NMR (400 MHz, DMSO-d6): δ 8.25 (d, J = 10.6 Hz, |
| | | | | 1H), 8.03 (s, 1H), 7.47 (s, 1H), 6.98 (s, 1H), 4.60 (d, J = |
| | | | | 15.3 Hz, 2H), 3.91 (d, J = 1.5 Hz, 3H), 3.83-3.73 (m, |
| | | | | 2H), 3.61 (s, 3H), 3.41 (s, 2H), 3.03 (s, 4H), 2.89 (t, J = |
| | | | | 5.6 Hz, 1H), 2.75 (t, J = 5.5 Hz, 1H), 2.56 (d, J = 16.5 Hz, |
| | | | | 4H), 2.11 (d, J = 5.9 Hz, 3H). |
| 529 | ** | * | 422.3 | 1H NMR (400 MHz, DMSO-d6): δ 8.25 (d, J = 10.5 Hz, |
| | | | | 1H), 8.03 (s, 1H), 7.66-7.56 (m, 2H), 6.45 (d, J = 0.8 |
| | | | | Hz, 1H), 4.61 (d, J = 15.2 Hz, 2H), 3.90 (d, J = 1.6 Hz, |
| | | | | 3H), 3.83-3.73 (m, 2H), 3.40 (s, 2H), 3.05 (s, 4H), 2.89 |
| | | | | (t, J = 5.6 Hz, 1H), 2.75 (t, J = 5.7 Hz, 1H), 2.60-2.52 |
| | | | | (m, 4H), 2.11 (d, J = 5.9 Hz, 3H). |
| 530 | * | * | 449.3 | 1H NMR (400 MHz, DMSO-d6): δ 11.33 (s, 1H), 8.27 (s, |
| | | | | 1H), 8.04 (d, J = 1.6 Hz, 1H), 7.37 (dt, J = 14.4, 7.2 Hz, |
| | | | | 1H), 6.20 (d, J = 9.0 Hz, 1H), 6.15 (s, 1H), 4.61 (d, J = |
| | | | | 15.6 Hz, 2H), 3.90 (s, 3H), 3.84-3.73 (m, 2H), 3.39 (s, |
| | | | | 2H), 3.07 (s, 4H), 2.89 (t, J = 5.7 Hz, 1H), 2.76 (t, J = 5.6 |
| | | | | Hz, 1H), 2.58 (d, J = 15.9 Hz, 4H), 2.12 (d, J = 5.9 Hz, 3H). |
| 531 | * | * | 449.3 | 1H NMR (400 MHz, DMSO-d6): δ 11.54 (s, 1H), 8.28 (s, |
| | | | | 1H), 8.04 (s, 1H), 7.48-7.40 (m, 1H), 7.31-7.23 (m, |
| | | | | 1H), 6.18 (t, J = 6.6 Hz, 1H), 4.61 (d, J = 15.6 Hz, 2H), |
| | | | | 3.91 (d, J = 1.6 Hz, 3H), 3.83-3.74 (m, 2H), 3.36 (s, |
| | | | | 2H), 3.07 (s, 4H), 2.89 (t, J = 5.6 Hz, 1H), 2.76 (t, J = 5.7 |
| | | | | Hz, 1H), 2.58 (s, 4H), 2.12 (d, J = 5.7 Hz, 3H). |
| 532 | * | * | 449.3 | 1H NMR (400 MHz, DMSO-d6): δ 11.46 (s, 1H), 8.26 (s, |
| | | | | 1H), 8.04 (s, 1H), 7.40 (dd, J = 9.4, 2.4 Hz, 1H), 7.24 (s, |
| | | | | 1H), 6.31 (d, J = 9.3 Hz, 1H), 4.61 (d, J = 15.3 Hz, 2H), |
| | | | | 3.90 (d, J = 1.4 Hz, 3H), 3.86-3.71 (m, 2H), 3.29 (s, |
| | | | | 2H), 3.05 (s, 4H), 2.89 (t, J = 5.6 Hz, 1H), 2.75 (t, J = 5.6 |
| | | | | Hz, 1H), 2.20-2.04 (m, 3H). |
| 533 | * | * | 463.3 | 1H NMR (400 MHz, DMSO-d6): δ 8.28 (s, 1H), 8.06 (d, |
| | | | | J = 1.9 Hz, 1H), 7.33 (dd, J = 9.1, 6.8 Hz, 1H), 6.34 (dd, |
| | | | | J = 9.1, 1.1 Hz, 1H), 6.26-6.18 (m, 1H), 4.61 (d, J = 15.7 |
| | | | | Hz, 2H), 3.91 (d, J = 1.6 Hz, 3H), 3.83-3.74 (m, 2H), |
| | | | | 3.54 (d, J = 3.3 Hz, 5H), 3.06 (s, 4H), 2.89 (t, J = 5.6 Hz, |
| | | | | 1H), 2.75 (t, J = 5.7 Hz, 1H), 2.61 (s, 4H), 2.12 (d, J = 5.3 |
| | | | | Hz, 3H). |
| 534 | * | * | 463.3 | 1H NMR (400 MHz, DMSO-d6): δ 8.28 (s, 1H), 8.04 (s, |
| | | | | 1H), 7.61 (d, J = 5.5 Hz, 1H), 7.43 (d, J = 6.2 Hz, 1H), |
| | | | | 6.21 (t, J = 6.7 Hz, 1H), 4.61 (d, J = 15.5 Hz, 2H), 3.91 (s, |
| | | | | 3H), 3.84-3.73 (m, 2H), 3.44 (s, 3H), 3.40 (s, 2H), 3.07 |
| | | | | (s, 4H), 2.89 (t, J = 5.5 Hz, 1H), 2.76 (t, J = 5.5 Hz, 1H), |
| | | | | 2.60 (s, 4H), 2.12 (d, J = 5.7 Hz, 3H). |
| 535 | * | * | 463.3 | 1H NMR (400 MHz, DMSO-d6): δ 8.26 (s, 1H), 8.04 (s, |
| | | | | 1H), 7.59 (d, J = 2.2 Hz, 1H), 7.38 (dd, J = 9.3, 2.4 Hz, |
| | | | | 1H), 6.36 (d, J = 9.2 Hz, 1H), 4.61 (d, J = 15.3 Hz, 2H), |
| | | | | 3.91 (d, J = 1.6 Hz, 3H), 3.78 (dd, J = 10.1, 5.7 Hz, 2H), |
| | | | | 3.40 (s, 3H), 3.27 (s, 2H), 3.05 (s, 4H), 2.88 (t, J = 5.7 Hz, |
| | | | | 1H), 2.75 (t, J = 5.7 Hz, 1H), 2.11 (d, J = 6.0 Hz, 3H). |
| 536 | * | * | 422.3 | 1H NMR (400 MHz, DMSO-d6): δ 8.25 (S, 1H), 8.02 (s, |
| | | | | 1H), 7.50(s, 2H), 4.62 (d, J = 15.3 Hz, 2H), 3.90 (S, 3H), |
| | | | | 3.78-3.75 (m, 2H), 3.43 (s, 2H), 3.03 (s, 4H), 2.89 (t, J = |
| | | | | 5.6 Hz, 1H), 2.76 (t, J = 5.5 Hz, 1H), 2.50(d, J = 16.5 Hz, |
| | | | | 4H), 2.33 (d, J = 3.6 Hz, 3H), 2.12(s, 2H) |
| 537 | ** | ** | 422.3 | 1H NMR (400 MHz, DMSO-d6): δ 8.25 (s, 1H), 8.02 (s, |
| | | | | 1H), 7.60 (d, J = 1.0 Hz, 1H), 6.41 (dd, J = 3.1, 1.9 Hz, |
| | | | | 1H), 6.31 (d, J = 3.0 Hz, 1H), 4.60 (d, J = 15.4 Hz, 2H), |
| | | | | 3.90 (d, J = 1.5 Hz, 3H), 3.84-3.72 (m, 2H), 3.56 (s, |
| | | | | 2H), 3.04 (s, 4H), 2.89 (t, J = 5.7 Hz, 1H), 2.75 (t, J = 5.7 |
| | | | | Hz, 1H), 2.56 (s, 4H), 2.11 (d, J = 6.0 Hz, 3H). |
| 538 | *** | ** | 480.3 | 1H NMR (400 MHz, DMSO-d6): δ 8.36 (s, 1H), 8.07 (d, |
| | | | | J = 2.6 Hz, 1H), 7.53 (d, J = 8.5 Hz, 2H), 7.46 (d, J = 8.5 |
| | | | | Hz, 2H), 4.63 (d, J = 16.5 Hz, 2H), 3.91 (d, J = 1.4 Hz, |
| | | | | 3H), 3.79 (m, 4H), 3.54 (s, 2H), 3.08 (s, 4H), 2.90 (t, J = |
| | | | | 5.7 Hz, 1H), 2.77 (t, J = 5.3 Hz, 1H), 2.12 (d, J = 5.2 Hz, 3H). |
| 539 | ** | ** | 494.2 | 1H NMR (400 MHz, DMSO-d6): δ 8.35 (s, 1H), 8.07 (d, |
| | | | | J = 2.6 Hz, 1H), 7.49 (d, J = 8.1 Hz, 1H), 7.42 (d, J = 1.3 |
| | | | | Hz, 1H), 7.27 (dd, J = 8.1, 1.7 Hz, 1H), 4.63 (d, J = 16.6 |
| | | | | Hz, 2H), 3.91 (d, J = 1.5 Hz, 3H), 3.86-3.73 (m, 4H), |
| | | | | 3.54 (s, 2H), 3.08 (s, 4H), 2.90 (t, J = 5.7 Hz, 1H), 2.77 (t, |
| | | | | J = 5.6 Hz, 1H), 2.37 (s, 3H), 2.12 (d, J = 5.3 Hz, 3H). |
| 540 | **** | ** | 483.4 | 1H NMR (400 MHz, ACN-d3) δ 8.17 (d, J = 2.0 Hz, 1H), |
| | | | | 8.08 (d, J = 4.6 Hz, 1H), 7.17 (td, J = 9.3, 5.5 Hz, 1H), |
| | | | | 7.01 (ddd, J = 11.6, 8.8, 3.1 Hz, 1H), 6.94-6.87 (m, 1H), |
| | | | | 4.73-4.54 (m, 2H), 4.49-4.43 (m, 1H), 3.92 (d, J = 1.8 |
| | | | | Hz, 3H), 3.89-3.86 (m, overlapping, 1H) 3.58-3.47 (m, |
| | | | | 1H), 3.47-3.39 (m, 2H), 3.06-2.99 (m, 2H), 2.98-2.94 |
| | | | | (m, overlapping, 1H), 2.14 (d, J = 3.4 Hz, 3H), 2.12-2.09 |
| | | | | (m, 2H), 1.93-1.84 (m, 2H), 1.31 (d, J = 7.0 Hz, 1.7H), |
| | | | | 1.27 (d, J = 4.9 Hz, 1.7H) |
| 541 | *** | ** | 481.3 | 1H NMR (400 MHz, DMSO-d6): δ 8.27 (s, 1H), 8.03 (s, |
| | | | | 1H), 7.91 (dd, J = 15.4, 8.6 Hz, 1H), 7.43 (ddd, J = 11.6, |
| | | | | 9.4, 2.4 Hz, 1H), 7.26 (td, J = 8.4, 2.3 Hz, 1H), 4.62 (d, |
| | | | | J = 16.0 Hz, 2H), 3.91 (d, J = 1.5 Hz, 3H), 3.83-3.76 (m, |
| | | | | 2H), 3.41 (dd, J = 20.6, 8.1 Hz, 2H), 3.24 (q, J = 7.5 Hz, |
| | | | | 2H), 2.90 (d, J = 5.6 Hz, 1H), 2.82 (t, J = 10.8 Hz, 1H), |
| | | | | 2.79-2.72 (m, 1H), 2.12 (d, J = 5.3 Hz, 3H), 1.91 (d, J = |
| | | | | 10.8 Hz, 2H), 1.75 (dd, J = 21.7, 10.6 Hz, 2H). |
| 542 | **** | *** | 465.4 | 1H NMR (400 MHz, DMSO-d6) δ 8.36 (s, 1H), 8.13- |
| | | | | 8.02 (m, 1H), 7.40-7.29 (m, 1H), 7.26-7.17 (m, 1H), |
| | | | | 7.11-7.00 (m, 1H), 6.23 (s, 1H), 4.66-4.57 (m, 2H), |
| | | | | 3.92 (s, 3H), 3.82-3.72 (m, 2H), 3.20-3.11 (m, 2H), |
| | | | | 3.10-3.02 (m, 2H), 2.93-2.81 (m, 1H), 2.78-2.71 (m, |
| | | | | 1H), 2.54-2.51 (submerged m, 2H), 2.47-2.41 |
| | | | | (submerged m, 2H), 2.13-2.08 (m, 3H). |
| 543 | ** | ** | 536.4 | 1H NMR (400 MHz, DMSO-d6) δ 7.36-7.24 (m, 2H), |
| | | | | 7.07-6.97 (m, 1H), 4.68-4.57 (m, 2H), 4.57-4.47 (m, |
| | | | | 1H), 3.87-3.65 (m, 4H), 3.56-3.45 (m, 1H), 3.43- |
| | | | | 3.24 (m, 4H, overlapping), 3.08-2.94 (m, 5H), 2.93- |
| | | | | 2.72 (m, 2H), 2.31-2.21 (m, 1H), 2.17-2.01 (m, 6H), |
| | | | | 1.90-1.74 (m, 2H). |
| 544 | ** | ** | 514.5 | 1H NMR (400 MHz, DMSO-d6) δ 7.36-7.25 (m, 2H), |
| | | | | 7.06-6.97 (m, 1H), 4.66-4.41 (m, 7H), 3.83-3.59 (m, |
| | | | | 4H), 3.32-3.23 (m, 2H, overlapping), 3.06 (t, J = 8.3 Hz, |
| | | | | 1H), 3.02-2.92 (m, 2H), 2.91-2.78 (m, 2H), 2.74 (t, J = |
| | | | | 5.7 Hz, 1H), 2.55-2.43 (m, 2H, overlapping), 2.28- |
| | | | | 2.13 (m, 1H), 2.13-2.09 (m, 3H), 2.09-1.87 (m, 3H), |
| | | | | 1.87-1.74 (m, 2H). |
| 545 | *** | ** | 483.4 | 1H-NMR (400 MHz, DMSO-d6) δ 8.25 (s, 1H), 8.02 (s, |
| | | | | 1H), 7.56-7.47 (m, 1H), 7.17 (td, J = 10.6, 2.4 Hz, 1H), |
| | | | | 7.09 (td, J = 8.5, 2.2 Hz, 1H), 5.43 (d, J = 4.8 Hz, 1H), |
| | | | | 4.70-4.63 (m, 1H), 4.61 (s, 1H), 4.57 (s, 1H), 3.91 (s, |
| | | | | 1.5H), 3.90 (s, 1.5H), 3.77 (dd, J = 10.2, 5.5 Hz, 2H), |
| | | | | 3.49-3.35 (overlapped m, 2H), 2.86 (t, J = 5.6 Hz, 1H), |
| | | | | 2.74 (t, J = 5.5 Hz, 1H), 2.62-2.52 (overlapped m, 2H), |
| | | | | 2.11 (s, 1.5H), 2.10 (s, 1.5H), 1.92-1.81 (m, 1H), 1.69- |
| | | | | 1.57 (m, 1H), 1.56-1.40 (m, 2H), 1.35-1.25 (m, 1H). |
| 546 | **** | ** | 473.4 | 1H NMR (400 MHz, DMSO-d6) δ 7.36-7.26 (m, 2H), |
| | | | | 7.06-6.98 (m, 1H), 4.58-4.47 (m, 3H, overlapped), |
| | | | | 3.82-3.66 (m, 2H), 3.61-3.54 (m, 1H), 3.52 (dd, J = |
| | | | | 10.3, 5.7 Hz, 1H), 3.46-3.38 (m, 1H), 3.29-3.24 (m, |
| | | | | 1H), 3.24 (s, 1.30H, rotamers), 3.23 (s, 1.71H, rotamers), |
| | | | | 3.13 (t, J = 9.8 Hz, 1H), 3.05-2.94 (m, 1H), 2.87-2.81 |
| | | | | (m, 1H), 2.74-2.68 (m, 1H), 2.14-2.01 (m, 6H, |
| | | | | overlapped), 1.89-1.74 (m, 2H), 1.64-1.53 (m, 1H), |
| | | | | 1.29-1.22 (m, 1H), 1.00-0.92 (m, 1H). |
| 547 | ** | ** | 473.5 | 1H NMR (400 MHz, DMSO-d6) δ 7.37-7.25 (m, 2H), |
| | | | | 7.06-6.98 (m, 1H), 4.58-4.46 (m, 3H, overlapped), |
| | | | | 3.82-3.67 (m, 2H), 3.62-3.55 (m, 1H), 3.52 (dd, J = |
| | | | | 10.3, 5.7 Hz, 1H), 3.46-3.37 (m, 1H), 3.29-3.24 (m, |
| | | | | 1H), 3.24 (s, 1.24H, rotamers), 3.23 (s, 1.70H, rotamers), |
| | | | | 3.13 (t, J = 9.8 Hz, 1H), 3.04-2.95 (m, 1H), 2.87-2.82 |
| | | | | (m, 1H), 2.73-2.68 (m, 1H), 2.13-2.01 (m, 6H, |
| | | | | overlapped), 1.88-1.74 (m, 2H), 1.64-1.55 (m, 1H), |
| | | | | 1.30-1.22 (m, 1H), 1.00-0.92 (m, 1H). |
| 548 | * | * | 422.3 | 1H NMR (400 MHz, DMSO-d6): δ 8.26 (s, 1H), 8.02 (s, |
| | | | | 1H), 7.54 (s, 1H), 6.88 (d, J = 77.7 Hz, 1H), 4.60 (d, J = |
| | | | | 15.4 Hz, 2H), 3.90 (d, J = 1.5 Hz, 3H), 3.83-3.72 (m, |
| | | | | 2H), 3.46 (t, J = 17.6 Hz, 2H), 3.04 (s, 4H), 2.89 (t, J = |
| | | | | 5.6 Hz, 1H), 2.75 (t, J = 5.6 Hz, 1H), 2.55(S, 1H), 2.11 (d, |
| | | | | J = 5.9 Hz, 3H). |
| 549 | *** | ** | 494.1 | 1H NMR (400 MHz, DMSO-d6): δ 8.39 (s, 1H), 8.08 (d, |
| | | | | J = 2.3 Hz, 1H), 7.53 (d, J = 8.5 Hz, 2H), 7.46 (d, J = 8.5 |
| | | | | Hz, 2H), 4.63 (d, J = 16.3 Hz, 2H), 4.21 (q, J = 7.1 Hz, |
| | | | | 2H), 3.79 (d, J = 3.8 Hz, 4H), 3.53 (s, 3H), 3.08 (s, 4H), |
| | | | | 2.90 (t, J = 5.6 Hz, 1H), 2.77 (t, J = 5.6 Hz, 1H), 2.12 (d, |
| | | | | J = 5.2 Hz, 3H), 1.41 (t, J = 7.2 Hz, 3H). |
| 550 | ** | ** | 508.2 | 1H NMR (400 MHz, DMSO-d6): δ 8.38 (d, J = 9.8 Hz, |
| | | | | 1H), 8.08 (d, J = 2.4 Hz, 1H), 7.49 (d, J = 8.2 Hz, 1H), |
| | | | | 7.43 (d, J = 1.4 Hz, 1H), 7.27 (dd, J = 8.1, 1.8 Hz, 1H), |
| | | | | 4.63 (d, J = 16.4 Hz, 2H), 4.21 (q, J = 7.2 Hz, 2H), 3.85- |
| | | | | 3.75 (m, 3H), 3.54 (s, 2H), 3.08 (s, 4 H), 2.90 (t, J = 5.7 |
| | | | | Hz, 1H), 2.77 (dd, J = 11.2, 5.5 Hz, 1H), 2.36 (d, J = 7.6 |
| | | | | Hz, 3H), 2.12 (d, J = 5.3 Hz, 3H), 1.41 (t, J = 7.3 Hz, 3H). |
| 551 | *** | * | 528.3 | 1H NMR (400 MHz, DMSO-d6): δ 8.24 (s, 1H), 8.01 (s, |
| | | | | 1H), 7.41-7.35 (m, 1H), 7.17-7.06 (m, 2H), 5.18 (s, |
| | | | | 1H), 4.86 (s, 1H), 4.59 (d, J = 16.1 Hz, 2H), 3.89 (d, J = |
| | | | | 1.3 Hz, 3H), 3.80-3.73 (m, 2H), 3.62 (s, 2H), 2.94- |
| | | | | 2.83 (m, 5H), 2.78 (s, 1H), 2.76-2.70 (m, 1H), 2.61 (d, |
| | | | | J = 4.7 Hz, 2H), 2.11 (d, J = 5.8 Hz, 3H). |
| 552 | ** | * | 510.3 | 1H NMR (400 MHz, DMSO-d6): δ 8.26 (s, 1H), 8.02 (s, |
| | | | | 1H), 7.67 (t, J = 7.2 Hz, 1H), 7.30 (dd, J = 13.0, 5.7 Hz, |
| | | | | 1H), 7.18 (t, J = 7.1 Hz, 1H), 7.09 (dd, J = 11.9, 8.0 Hz, |
| | | | | 1H), 5.02 (s, 1H), 4.61 (d, J = 16.0 Hz, 2H), 3.91 (s, 3H), |
| | | | | 3.79 (d, J = 3.9 Hz, 2H), 3.70 (d, J = 11.1 Hz, 1H), 3.60 |
| | | | | (d, J = 11.1 Hz, 1H), 2.87 (d, J = 12.8 Hz, 7H), 2.75 (d, |
| | | | | J = 5.3 Hz, 1H), 2.59 (d, J = 10.1 Hz, 2H), 2.13 (d, J = 6.0 |
| | | | | Hz, 3H). |
| 553 | *** | ** | 497.4 | 1H-NMR (400 MHz, DMSO-d6) δ 8.25 (s, 1H), 8.02 (s, |
| | | | | 1H), 7.46-7.37 (m, 1H), 7.30-7.21 (m, 1H), 7.14 (td, |
| | | | | J = 8.5, 2.3 Hz, 1H), 4.61 (s, 1H), 4.57 (s, 1H), 4.30 (d, J = |
| | | | | 7.9 Hz, 1H), 3.91 (s, 1.5H), 3.90 (s, 1.5H), 3.77 (dd, J = |
| | | | | 10.2, 5.7 Hz, 2H), 3.48-3.39 (m, 1H), 3.37-3.30 |
| | | | | (overlapped m, 1H), 3.13 (s, 3H), 2.86 (t, J = 5.8 Hz, 1H), |
| | | | | 2.73 (t, J = 5.7 Hz, 1H), 2.65-2.51 (overlapped m, 2H), |
| | | | | 2.11 (s, 1.5H), 2.10 (s, 1.5H), 2.01-1.93 (m, 1H), 1.80- |
| | | | | 1.67 (m, 1H), 1.58-1.34 (m, 2H), 1.28-1.19 (m, 1H). |
| 554 | **** | ** | 547.3 | 1H NMR (400 MHz, DMSO-d6): δ 9.10 (s, 1H), 8.96 (s, |
| | | | | 1H), 8.47 (s, 1H), 7.96 (s, 1H), 7.74 (s, 1H), 7.36-7.26 |
| | | | | (m, 2H), 7.05-6.99 (m, 1H), 5.50 (d, J = 4.4 Hz, 1H), |
| | | | | 4.74 (s, 1H), 4.57 (dd, J = 7.5, 4.0 Hz, 1H), 4.53 (d, J = |
| | | | | 3.4 Hz, 2H), 3.64 (d, J = 13.0 Hz, 2H), 2.07 (s, 2H), 1.84 |
| | | | | (dd, J = 8.5, 4.0 Hz, 2H), 1.06 (t, J = 8.3 Hz, 6H). |
| 555 | *** | ** | 547.3 | 1H NMR (400 MHz, DMSO-d6): δ 9.10 (s, 1H), 8.96 (s, |
| | | | | 1H), 8.47 (s, 1H), 7.96 (s, 1H), 7.74 (s, 1H), 7.30 (ddd, |
| | | | | J = 11.7, 9.2, 3.5 Hz, 2H), 7.05-6.98 (m, 1H), 5.50 (d, J = |
| | | | | 4.7 Hz, 1H), 4.75 (s, 1H), 4.57 (d, J = 3.8 Hz, 1H), 4.54 |
| | | | | (d, J = 3.7 Hz, 1H), 3.62 (s, 2H), 3.31-3.20 (m, 11H), |
| | | | | 2.07 (s, 3H), 1.84 (dd, J = 8.3, 4.0 Hz, 3H), 1.06 (t, J = |
| | | | | 8.2 Hz, 7H). |
| 556 | *** | ** | 497.4 | 1H NMR (400 MHz, DMSO-d6) δ 9.06 (d, J = 0.5 Hz, |
| | | | | 1H), 8.50 (s, 1H), 8.16 (s, 1H), 7.82 (d, J = 0.4 Hz, 1H), |
| | | | | 7.36-7.25 (m, 2H), 7.07-6.97 (m, 1H), 5.20 (s, 1H), |
| | | | | 4.61 (overlapping t, J = 6.0 Hz, 1H), 4.59-4.54 |
| | | | | (overlapping m, 1H), 3.95 (s, 3H), 3.72-3.55 (m, 4H), |
| | | | | 3.29-3.19 (m, 2H), 2.14-2.02 (m, 2H), 1.91-1.79 (m, |
| | | | | 2H), 1.44 (s, 3H). |
| 557 | **** | **** | 485.4 | 1H NMR (400 MHz, DMSO-d6) δ 8.27 (s, 1H), 8.08- |
| | | | | 8.00 (m, 1H), 7.56 (dd, J = 15.2, 8.4 Hz, 1H), 7.38-7.28 |
| | | | | (m, 1H), 7.23-7.12 (m, 1H), 5.59 (dd, J = 46.2, 8.3 Hz, |
| | | | | 1H), 4.67-4.53 (m, 2H), 3.94-3.88 (m, 3H), 3.81- |
| | | | | 3.72 (m, 2H), 3.52-3.34 (m, 2H), 2.87 (t, J = 5.8 Hz, |
| | | | | 1H), 2.79-2.55 (m, 3H, overlapping), 2.16-1.99 (m, |
| | | | | 4H), 1.98-1.88 (m, 1H), 1.74-1.56 (m, 1H), 1.54- |
| | | | | 1.37 (m, 1H), 1.32-1.19 (m, 1H). |
| 558 | ** | *** | 497.4 | 1H NMR (400 MHz, DMSO-d6) δ 8.24-8.18 (m, 1H), |
| | | | | 8.02 (s, 1H), 7.71-7.59 (m, 1H), 7.19-7.00 (m, 2H), |
| | | | | 5.21 (s, 1H), 4.69-4.50 (m, 2H), 3.96-3.84 (m, 3H), |
| | | | | 3.83-3.69 (m, 2H), 3.56-3.44 (m, 1H), 3.34-3.20 (m, |
| | | | | 1H, overlapping), 2.86 (t, J = 5.8 Hz, 1H), 2.72 (t, J = 5.7 |
| | | | | Hz, 1H), 2.65-2.54 (m, 1H), 2.48-2.41 (m, 1H), 2.16- |
| | | | | 2.05 (m, 3H), 1.88-1.74 (m, 2H), 1.73-1.58 (m, 1H), |
| | | | | 1.57-1.44 (m, 4H), 1.09-0.96 (m, 1H). |
| 559 | *** | ** | 528.4 | 1H-NMR (400 MHz, DMSO-d6) δ 9.09 (s, 1H), 8.90 (s, |
| | | | | 1H), 8.43 (s, 1H), 7.95 (t, J = 58.7 Hz, 1H), 7.72 (s, 1H), |
| | | | | 7.43 (td, J = 7.7, 1.7 Hz, 1H), 7.35-7.29 (m, 1H), 7.22- |
| | | | | 7.10 (m, 2H), 5.51 (d, J = 5.4 Hz, 1H), 4.75 (s, 1H), 4.52 |
| | | | | (d, J = 5.4 Hz, 1H), 3.60 (s, 2H), 3.39-3.34 (overlapping |
| | | | | m, 4H), 2.60-2.53 (m, 4H), 1.06 (d, J = 7.6 Hz, 6 H). |
| 560 | **** | *** | 447.4 | 1H NMR (400 MHz, DMSO-d6) δ 8.36 (s, 1H), 8.09- |
| | | | | 8.06 (m, 1H), 7.31-7.24 (m, 2H), 7.19-7.13 (m, 2H), |
| | | | | 6.36 (s, 1H), 4.67-4.57 (m, 2H), 3.95-3.90 (m, 3H), |
| | | | | 3.82-3.73 (m, 2H), 3.34 (s, 2H under water), 3.19-3.10 |
| | | | | (m, 2H), 3.09-3.02 (m, 2H), 2.92-2.72 (m, 2H), 2.62- |
| | | | | 2.56 (m, 2H), 2.16-2.07 (m, 3H). |
| 561 | **** | *** | 511.5 | 1H NMR (400 MHz, DMSO-d6): δ 9.02 (s, 1H), 8.61 (s, |
| | | | | 1H), 8.27 (s, 1H), 7.83 (s, 1H), 7.37-7.25 (m, 2H), 7.08- |
| | | | | 6.98 (m, 1H), 5.47 (d, J = 5.3 Hz, 1H), 4.68 (s, 1H), |
| | | | | 4.57 (m, 1H), 4.54 (d, J = 5.3 Hz, 1H), 3.97 (s, 3H), 3.55 |
| | | | | (m, 2H), 3.18 (m, 2H), 2.10 (m, 2H), 1.89 (m, 2H), 1.06 |
| | | | | (d, J = 9.7 Hz, 6H). |
| 562 | **** | *** | 511.4 | 1H NMR (400 MHz, DMSO-d6): δ 9.02 (s, 1H), 8.61 (s, |
| | | | | 1H), 8.27 (s, 1H), 7.83 (s, 1H), 7.37-7.25 (m, 2H), 7.08- |
| | | | | 6.98 (m, 1H), 5.47 (d, J = 5.3 Hz, 1H), 4.68 (s, 1H), |
| | | | | 4.57 (m, 1H), 4.54 (d, J = 5.3 Hz, 1H), 3.97 (s, 3H), 3.55 |
| | | | | (m, 2H), 3.18 (m, 2H), 2.10 (m, 2H), 1.89 (m, 2H), 1.06 |
| | | | | (d, J = 9.7 Hz, 6H). |
| 563 | *** | ** | 508.4 | 1H NMR (400 MHz, DMSO-d6) δ 9.10 (s, 1H), 8.56 (s, |
| | | | | 1H), 8.31 (s, 1H), 8.22 (s, 1H), 7.53-7.42 (m, 1H), 7.26- |
| | | | | 7.15 (m, 1H), 7.13-7.02 (m, 1H), 5.91 (s, 1H), 3.96 (s, |
| | | | | 3H), 3.58 (s, 2H), 3.46-3.38 (m, 4H), 2.62-2.55 (m, |
| | | | | 4H), 1.55 (s, 6H). |
| 564 | *** | ** | 497.4 | 1H NMR (400 MHz, DMSO-d6) δ 9.06 (s, 1H), 8.50 (s, |
| | | | | 1H), 8.16 (s, 1H), 7.82 (s, 1H), 7.39-7.24 (m, 2H), 7.09- |
| | | | | 6.96 (m, 1H), 5.21 (s, 1H), 4.62 (overlapping t, J = 6.0 |
| | | | | Hz, 1H), 4.60-4.50 (overlapping m, 1H), 3.95 (s, 3H), |
| | | | | 3.75-3.55 (m, 4H), 3.30-3.19 (m, 2H), 2.15-2.01 (m, |
| | | | | 2H), 1.90-1.76 (m, 2H), 1.44 (s, 3H) |
| 565 | ** | ** | 497.4 | 1H NMR (400 MHz, DMSO-d6) δ 9.06 (s, 1H), 8.50 (s, |
| | | | | 1H), 8.16 (s, 1H), 7.82 (s, 1H), 7.39-7.23 (m, 2H), 7.07- |
| | | | | 6.98 (m, 1H), 5.21 (s, 1H), 4.61 (overlapping t, J = 6.1 |
| | | | | Hz, 1H), 4.59-4.52 (overlapping m, 1H), 3.95 (s, 3H), |
| | | | | 3.72-3.52 (m, 4H), 3.29-3.18 (m, 2H), 2.16-1.99 (m, |
| | | | | 2H), 1.93-1.76 (m, 2H), 1.44 (s, 3H) |
| 566 | *** | ** | 544.2 | 1H-NMR (400 MHz, DMSO-d6) δ 9.20 (s, 1H), 8.96 (s, |
| | | | | 1H), 8.46 (s, 1H), 8.16 (s, 1H), 7.95 (t, J = 58.6 Hz, 1H), |
| | | | | 7.52-7.42 (m, 1H), 7.21 (td, J = 10.1, 2.5 Hz, 1H), 7.07 |
| | | | | (td, J = 8.6, 2.4 Hz, 1H), 5.91 (s, 1H), 3.56 (s, 2H), 3.48- |
| | | | | 3.39 (m, 4H), 2.61-2.53 (overlapped m, 4H), 1.55 (s, 6H). |
| 567 | *** | ** | 511.4 | 1H NMR (400 MHz, DMSO-d6) δ 9.06 (d, J = 0.6 Hz, |
| | | | | 1H), 8.50 (s, 1H), 8.16 (d, J = 0.5 Hz, 1H), 7.82 (d, J = |
| | | | | 0.7 Hz, 1H), 7.36-7.26 (m, 2H), 7.05-6.98 (m, 1H), |
| | | | | 5.37 (s, 1H), 4.63-4.54 (m, 1H), 3.95 (s, 3H), 3.67 |
| | | | | (overlapping d, J = 9.4 Hz, 1H), 3.64-3.61 (overlapping |
| | | | | m, 2H), 3.56 (d, J = 9.4 Hz, 1H), 3.30-3.21 (overlapping |
| | | | | m, 2H), 3.20 (overlapping s, 3H), 2.14-2.03 (m, 2H), |
| | | | | 1.91-1.78 (m, 2H), 1.45 (s, 3H). |
| 568 | *** | ** | 526.4 | 1H NMR (400 MHz, DMSO-d6) δ 9.18 (d, J = 0.6 Hz, |
| | | | | 1H), 8.88 (s, 1H), 8.36 (s, 1H), 8.15 (d, J = 0.6 Hz, 1H), |
| | | | | 7.47 (m, 1H), 7.21 (td, J = 10.2, 2.5 Hz, 1H), 7.07 (td, J = |
| | | | | 8.5, 2.1 Hz, 1H), 6.27 (d, J = 52.3 Hz, 2H), 5.93 (s, 1H), |
| | | | | 3.56 (s, 2H), 3.45 (m, 4H), 2.56 (m, 4H), 1.55 (s, 6H). |
| 569 | ** | * | 510.33 | 1H-NMR (400 MHz, DMSO-d6) δ 8.45 (s, 1H), 8.39 (bs, |
| | | | | formate salt, 1H), 8.20 (d, J = 8.5 Hz, 1H), 8.14 (d, J = |
| | | | | 0.6 Hz, 1H), 7.71 (d, J = 8.5 Hz, 1H), 7.53-7.43 (m, |
| | | | | 1H), 7.27-7.17 (m, 1H), 7.12-7.02 (m, 1H), 4.51 (s, |
| | | | | 1H), 3.94 (s, 3H), 3.58 (s, 2H), 3.39-3.19 (m, 4H), 2.64- |
| | | | | 2.54 (m, 4H), 1.09 (overlapping s, 3H), 1.06 |
| | | | | (overlapping s, 3H). |
| 570 | **** | ** | 525.4 | 1H NMR (400 MHz, DMSO-d6): δ 9.04 (s, 1H), 8.53 (s, |
| | | | | 1H), 8.16 (s, 1H), 7.71 (s, 1H), 7.36-7.26 (m, 2H), 7.05- |
| | | | | 6.98 (m, 1H), 5.47 (d, J = 5.4 Hz, 1H), 4.76 (s, 1H), |
| | | | | 4.57 (s, 1H), 4.52 (d, J = 5.3 Hz, 1H), 4.26 (q, J = 7.2 Hz, |
| | | | | 2H), 3.70-3.60 (m, 2H), 3.25 (d, J = 12.8 Hz, 2H), 2.08 |
| | | | | (s, 2H), 1.90-1.79 (m, 3H), 1.43 (t, J = 7.2 Hz, 4H), 1.07 |
| | | | | (d, J = 14.5 Hz, 6H). |
| 571 | **** | ** | 525.8 | 1H NMR (400 MHz, DMSO-d6): δ 9.04 (s, 1H), 8.53 (s, |
| | | | | 1H), 8.16 (s, 1H), 7.71 (s, 1H), 7.37-7.26 (m, 2H), 7.08- |
| | | | | 6.97 (m, 1H), 5.48 (d, J = 5.4 Hz, 1H), 4.76 (s, 1H), |
| | | | | 4.60-4.54 (m, 1H), 4.52 (d, J = 5.3 Hz, 1H), 4.25 (t, J = |
| | | | | 7.2 Hz, 2H), 3.70-3.61 (m, 2H), 3.28-3.21 (m, 2H), |
| | | | | 2.08 (s, 2H), 1.85 (dd, J = 8.3, 3.9 Hz, 2H), 1.43 (t, J = |
| | | | | 7.3 Hz, 3H), 1.07 (d, J = 14.5 Hz, 6H). |
| 572 | *** | ** | 537.5 | 1H NMR (400 MHz, DMSO-d6): δ 9.04 (s, 1H), 8.53 (s, |
| | | | | 1H), 8.13 (s, 1H), 7.70 (s, 1H), 7.35-7.25 (m, 2H), 7.02 |
| | | | | (ddd, J = 11.1, 3.9, 2.4 Hz, 1H), 5.47 (d, J = 5.4 Hz, 1H), |
| | | | | 4.76 (s, 1H), 4.61-4.54 (m, 1H), 4.52 (d, J = 5.4 Hz, |
| | | | | 1H), 3.91 (ddd, J = 11.2, 7.5, 3.9 Hz, 1H), 3.65 (d, J = |
| | | | | 13.8 Hz, 2H), 3.31-3.21 (m, 2H), 2.07 (s, 2H), 1.82 (dd, |
| | | | | J = 8.4, 3.9 Hz, 2H), 1.18-1.13 (m, 2H), 1.08 (d, J = 8.8 |
| | | | | Hz, 6H), 1.06-1.00 (m, 2H). |
| 573 | *** | ** | 537.5 | 1H NMR (400 MHz, DMSO-d6): δ 9.04 (s, 1H), 8.53 (s, |
| | | | | 1H), 8.13 (s, 1H), 7.70 (s, 1H), 7.35-7.25 (m, 2H), 7.02 |
| | | | | (ddd, J = 11.1, 3.9, 2.4 Hz, 1H), 5.47 (d, J = 5.4 Hz, 1H), |
| | | | | 4.76 (s, 1H), 4.61-4.54 (m, 1H), 4.52 (d, J = 5.4 Hz, |
| | | | | 1H), 3.91 (ddd, J = 11.2, 7.5, 3.9 Hz, 1H), 3.65 (d, J = |
| | | | | 13.8 Hz, 2H), 3.31-3.21 (m, 2H), 2.07 (s, 2H), 1.82 (dd, |
| | | | | J = 8.4, 3.9 Hz, 2H), 1.18-1.13 (m, 2H), 1.08 (d, J = 8.8 |
| | | | | Hz, 6H), 1.06-1.00 (m, 2H). |
| 574 | *** | ** | 524.3 | 1H NMR (400 MHz, DMSO-d6): δ 9.03 (s, 1H), 8.47 (s, |
| | | | | 1H), 8.13 (s, 1H), 7.70 (s, 1H), 7.48 (dd, J = 15.4, 8.5 Hz, |
| | | | | 1H), 7.21 (td, J = 10.1, 2.5 Hz, 1H), 7.08 (td, J = 8.4, 2.1 |
| | | | | Hz, 1H), 5.47 (d, J = 5.4 Hz, 1H), 4.75 (s, 1H), 4.51 (d, |
| | | | | J = 5.4 Hz, 1H), 4.25 (q, J = 7.3 Hz, 2H), 3.58 (s, 2H), 3.35 |
| | | | | (d, J = 7.2 Hz, 4H), 2.56 (d, J = 13.3 Hz, 4H), 1.43 (t, J = |
| | | | | 7.3 Hz, 3H), 1.07 (d, J = 12.8 Hz, 6H). |
| 575 | **** | ** | 524.3 | 1H NMR (400 MHz, DMSO-d6): δ 9.03 (s, 1H), 8.47 (s, |
| | | | | 1H), 8.13 (s, 1H), 7.69 (s, 1H), 7.48 (dd, J = 15.4, 8.5 Hz, |
| | | | | 1H), 7.21 (td, J = 10.2, 2.5 Hz, 1H), 7.07 (dt, J = 8.5, 4.2 |
| | | | | Hz, 1H), 5.47 (d, J = 5.4 Hz, 1H), 4.75 (s, 1H), 4.51 (d, |
| | | | | J = 5.4 Hz, 1H), 4.24 (q, J = 7.2 Hz, 2H), 3.58 (s, 2H), 3.35 |
| | | | | (d, J = 6.6 Hz, 4H), 2.57 (s, 4H), 1.43 (t, J = 7.3 Hz, 3H), |
| | | | | 1.06 (d, J = 12.8 Hz, 6H). |
| 576 | *** | ** | 536.4 | 1H NMR (400 MHz, DMSO-d6): δ 9.03 (s, 1H), 8.47 (s, |
| | | | | 1H), 8.10 (s, 1H), 7.69 (s, 1H), 7.48 (dd, J = 15.3, 8.4 Hz, |
| | | | | 1H), 7.22 (td, J = 10.0, 2.4 Hz, 1H), 7.08 (td, J = 8.5, 2.3 |
| | | | | Hz, 1H), 5.47 (d, J = 5.2 Hz, 1H), 4.75 (s, 1H), 4.51 (d, |
| | | | | J = 5.4 Hz, 1H), 3.87 (td, J = 7.4, 3.7 Hz, 1H), 3.58 (s, 2H), |
| | | | | 3.36 (d, J = 12.1 Hz, 4H), 2.53 (dd, J = 9.6, 7.7 Hz, 4H), |
| | | | | 1.12 (dd, J = 7.4, 3.7 Hz, 2H), 1.08 (s, 3H), 1.06-1.03 |
| | | | | (m, 3H), 1.02 (d, J = 1.5 Hz, 2H). |
| 577 | *** | ** | 536.4 | 1H NMR (400 MHz, DMSO-d6): δ 9.03 (s, 1H), 8.47 (s, |
| | | | | 1H), 8.10 (s, 1H), 7.69 (s, 1H), 7.48 (dd, J = 15.4, 8.5 Hz, |
| | | | | 1H), 7.22 (td, J = 10.1, 2.5 Hz, 1H), 7.08 (td, J = 8.5, 2.2 |
| | | | | Hz, 1H), 5.47 (d, J = 5.4 Hz, 1H), 4.75 (s, 1H), 4.51 (d, |
| | | | | J = 5.4 Hz, 1H), 3.88 (ddd, J = 11.3, 7.5, 3.9 Hz, 1H), 3.58 |
| | | | | (s, 2H), 3.35 (d, J = 2.4 Hz, 4H), 2.55 (d, J = 11.9 Hz, |
| | | | | 4H), 1.13 (dt, J = 7.8, 4.0 Hz, 2H), 1.08 (s, 3H), 1.07- |
| | | | | 1.03 (m, 3H), 1.02 (d, J = 1.5 Hz, 2H). |
| 578 | *** | ** | 510.3 | 1H NMR (400 MHz, DMSO-d6): δ 9.03 (s, 1H), 8.44 (s, |
| | | | | 1H), 8.12 (s, 1H), 7.70 (s, 1H), 7.48 (dd, J = 15.4, 8.5 Hz, |
| | | | | 1H), 7.24-7.17 (m, 1H), 7.07 (dt, J = 8.4, 4.2 Hz, 1H), |
| | | | | 5.47 (d, J = 5.4 Hz, 1H), 4.76 (s, 1H), 4.51 (d, J = 5.4 Hz, |
| | | | | 1H), 3.95 (s, 3H), 3.58 (s, 2H), 3.35 (d, J = 8.3 Hz, 4H), |
| | | | | 2.56 (d, J = 12.9 Hz, 4H), 1.07 (d, J = 12.9 Hz, 6H). |
| 579 | **** | *** | 510.3 | 1H NMR (400 MHz, DMSO-d6): δ 9.03 (s, 1H), 8.44 (s, |
| | | | | 1H), 8.12 (s, 1H), 7.69 (s, 1H), 7.48 (dd, J = 15.4, 8.5 Hz, |
| | | | | 1H), 7.21 (td, J = 10.1, 2.5 Hz, 1H), 7.08 (td, J = 8.5, 2.2 |
| | | | | Hz, 1H), 5.47 (d, J = 5.4 Hz, 1H), 4.75 (s, 1H), 4.51 (d, |
| | | | | J = 5.4 Hz, 1H), 3.95 (s, 3H), 3.58 (s, 2H), 3.35 (d, J = 5.9 |
| | | | | Hz, 4H), 2.57 (s, 4H), 1.06 (d, J = 12.8 Hz, 6H). |
| 580 | **** | ** | 530.3 | 1H NMR (400 MHz, DMSO-d6): δ 7.51 (d, J = 2.6 Hz, |
| | | | | 1H), 7.45 (dd, J = 8.3, 5.7 Hz, 2H), 7.35 (dd, J = 8.5, 2.6 |
| | | | | Hz, 1H), 6.91 (dd, J = 8.0, 2.7 Hz, 1H), 4.58 (d, J = 18.1 |
| | | | | Hz, 2H), 3.78 (dd, J = 11.5, 5.7 Hz, 2H), 3.55 (s, 2H), |
| | | | | 3.13 (s, 4H), 2.89 (t, J = 5.7 Hz, 1H), 2.75 (t, J = 5.6 Hz, |
| | | | | 1H), 2.44 (s, 1H), 2.11 (d, J = 11.4 Hz, 3H). |
| 581 | **** | ** | 498.5 | 1H NMR (400 MHz, DMSO-d6): δ 8.16 (s, 1H), 7.46- |
| | | | | 7.40 (m, 1H), 7.26-7.19 (m, 2H), 7.18-7.11 (m, 1H), |
| | | | | 6.93-6.88 (m, 1H), 6.33 (s, 2H), 4.58 (d, J = 18.1 Hz, |
| | | | | 2H), 3.78 (dd, J = 11.4, 5.7 Hz, 2H), 3.51 (s, 2H), 3.11 (s, |
| | | | | 4H), 3.03-3.00 (m, 4H), 2.88 (t, J = 5.6 Hz, 1H), 2.75 (t, |
| | | | | J = 5.7 Hz, 1H), 2.40 (s, 4H), 2.10 (t, J = 9.0 Hz, 3H). |
| 582 | **** | ** | 514.3 | 1H NMR (400 MHz, DMSO-d6): δ 8.13 (s, 1H), 7.44- |
| | | | | 7.35 (m, 3H), 7.24-7.20 (m, 1H), 6.90 (d, 1H), 6.33 (s, |
| | | | | 2H), 4.58 (d, J = 18.1 Hz, 2H), 3.78 (dd, J = 11.4, 5.7 Hz, |
| | | | | 2H), 3.51 (s, 2H), 3.11 (s, 4H), 3.03-3.00 (m, 4H), 2.88 |
| | | | | (t, J = 5.6 Hz, 1H), 2.75 (t, J = 5.7 Hz, 1H), 2.40 (s, 4H), |
| | | | | 2.10 (t, J = 9.0 Hz, 3H). |
| 583 | **** | ** | 547.3 | 1H NMR (400 MHz, DMSO-d6): δ 9.08 (d, J = 4.4 Hz, |
| | | | | 2H), 8.57 (s, 1H), 8.17-7.81 (m, 2H), 7.39-7.23 (m, |
| | | | | 2H), 7.02 (ddd, J = 4.7, 3.8, 1.7 Hz, 1H), 5.50 (d, J = 5.3 |
| | | | | Hz, 1H), 4.65 (s, 1H), 4.57 (d, J = 5.3 Hz, 2H), 3.54 (m, |
| | | | | 2H), 3.18 (m, 2H), 2.09 (s, 2H), 1.89 (m, 2H), 1.07 (d, J = |
| | | | | 3.7 Hz, 6H). |
| 584 | **** | ** | 547.3 | 1H NMR (400 MHz, DMSO-d6): δ 9.08 (d, J = 4.4 Hz, |
| | | | | 2H), 8.57 (s, 1H), 8.19-7.78 (m, 2H), 7.40-7.23 (m, |
| | | | | 2H), 7.02 (ddd, J = 4.7, 3.8, 1.7 Hz, 1H), 5.50 (d, J = 5.3 |
| | | | | Hz, 1H), 4.65 (s, 1H), 4.58 (dd, J = 9.0, 4.5 Hz, 2H), 3.55 |
| | | | | (d, J = 13.1 Hz, 2H), 3.25-3.12 (m, 2H), 2.09 (s, 2H), |
| | | | | 1.88 (dd, J = 8.4, 3.7 Hz, 2H), 1.07 (d, J = 3.8 Hz, 6H). |
| 585 | **** | *** | 546.3 | 1H NMR (400 MHz, DMSO-d6): δ 9.07 (s, 1H), 9.01 (s, |
| | | | | 1H), 8.53 (s, 1H), 8.13-7.81 (m, 2H), 7.48 (dd, J = 15.4, |
| | | | | 8.5 Hz, 1H), 7.22 (td, J = 10.0, 2.4 Hz, 1H), 7.08 (td, J = |
| | | | | 8.5, 2.3 Hz, 1H), 5.49 (d, J = 5.3 Hz, 1H), 4.64 (s, 1H), |
| | | | | 4.56 (d, J = 5.3 Hz, 1H), 3.60 (s, 2H), 3.28 (s, 4H), 2.60 |
| | | | | (s, 1H), 1.23(S, 1H) 1.06 (d, J = 3.7 Hz, 7H). |
| 586 | **** | *** | 546.3 | 1H NMR (400 MHz, DMSO-d6): δ 9.07 (s, 1H), 9.01 (s, |
| | | | | 1H), 8.53 (s, 1H), 8.12-7.82 (m, 2H), 7.48 (dd, J = 15.1, |
| | | | | 8.1 Hz, 1H), 7.22 (td, J = 10.2, 2.5 Hz, 1H), 7.08 (td, J = |
| | | | | 8.5, 2.1 Hz, 1H), 5.49 (d, J = 5.3 Hz, 1H), 4.64 (s, 1H), |
| | | | | 4.56 (d, J = 5.2 Hz, 1H), 3.60 (s, 2H), 3.28 (s, 4H), 2.60 |
| | | | | (s, 4H), 1.23(S, 1H), 1.06 (d, J = 3.6 Hz, 6H). |
| 587 | ** | ** | 511.3 | 1H NMR (400 MHz, DMSO-d6) δ 8.51 (s, 1H), 8.22 (d, |
| | | | | J = 8.4 Hz, 1H), 8.18 (s, 1H), 7.72 (d, J = 8.5 Hz, 1H), 7.37- |
| | | | | 7.24 (m, 2H), 7.07-6.97 (m, 1H), 5.60 (d, J = 5.3 Hz, |
| | | | | 1H), 4.72 (s, 1H), 4.62-4.54 (overlapping m, 1H), 4.52 |
| | | | | (overlapping d, J = 5.1 Hz, 1H), 3.96 (s, 3H), 3.66-3.54 |
| | | | | (m, 2H), 3.26-3.14 (m, 2H), 2.16-2.01 (m, 2H), 1.95- |
| | | | | 1.78 (m, 2H), 1.09 (d, J = 15.6 Hz, 6H). |
| 588 | **** | *** | 525.4 | 1H NMR (400 MHz, DMSO-d6): δ 9.02 (s, 1H), 8.65 (s, |
| | | | | 1H), 8.27 (s, 1H), 7.83 (s, 1H), 7.36-7.27 (m, 2H), 7.05- |
| | | | | 6.98 (m, 1H), 5.47 (d, J = 5.3 Hz, 1H), 4.67 (s, 1H), |
| | | | | 4.61-4.56 (m, 1H), 4.54 (d, J = 5.3 Hz, 1H), 4.28 (q, J = |
| | | | | 7.2 Hz, 2H), 3.55 (s, 2H), 3.18 (d, J = 7.7 Hz, 3H), 2.09 |
| | | | | (d, J = 11.4 Hz, 2H), 1.95-1.84 (m, 3H), 1.44 (t, J = 7.3 |
| | | | | Hz, 3H), 1.06 (d, J = 9.8 Hz, 6H). |
| 589 | **** | *** | 525.4 | 1H NMR (400 MHz, DMSO-d6): δ 9.02 (s, 1H), 8.65 (s, |
| | | | | 1H), 8.27 (s, 1H), 7.83 (s, 1H), 7.36-7.26 (m, 2H), 7.06- |
| | | | | 6.99 (m, 1H), 5.47 (d, J = 5.3 Hz, 1H), 4.67 (s, 1H), |
| | | | | 4.62-4.56 (m, 1H), 4.54 (d, J = 5.2 Hz, 1H), 4.28 (q, J = |
| | | | | 7.2 Hz, 2H), 3.55 (s, 2H), 3.18 (d, J = 7.7 Hz, 2H), 2.09 |
| | | | | (d, J = 11.4 Hz, 2H), 1.89 (dd, J = 8.3, 3.7 Hz, 2H), 1.44 |
| | | | | (t, J = 7.3 Hz, 3H), 1.06 (d, J = 9.8 Hz, 6H). |
| 590 | **** | *** | 537.4 | 1H NMR (400 MHz, DMSO-d6): δ 9.02 (s, 1H), 8.64 (s, |
| | | | | 1H), 8.25 (s, 1H), 7.83 (s, 1H), 7.36-7.25 (m, 2H), 7.05- |
| | | | | 6.98 (m, 1H), 5.47 (d, J = 5.3 Hz, 1H), 4.67 (s, 1H), |
| | | | | 4.58 (d, J = 4.0 Hz, 1H), 4.54 (d, J = 5.3 Hz, 2H), 3.93 |
| | | | | (td, J = 7.4, 3.7 Hz, 1H), 3.55 (s, 2H), 3.19 (dd, J = 10.8, |
| | | | | 6.0 Hz, 2H), 2.17-2.07 (m, 3H), 1.87 (d, J = 8.5 Hz, |
| | | | | 2H), 1.16 (dt, J = 7.1, 4.3 Hz, 2H), 1.11-1.01 (d, 6H). |
| 591 | **** | ** | 537.4 | 1H NMR (400 MHz, DMSO-d6): δ 9.02 (s, 1H), 8.64 (s, |
| | | | | 1H), 8.25 (s, 1H), 7.83 (s, 1H), 7.36-7.27 (m, 2H), 7.05- |
| | | | | 6.99 (m, 1H), 5.47 (d, J = 5.3 Hz, 1H), 4.67 (s, 1H), |
| | | | | 4.59 (d, J = 3.7 Hz, 1H), 4.54 (d, J = 5.3 Hz, 1H), 3.93 |
| | | | | (ddd, J = 11.2, 7.5, 3.9 Hz, 1H), 3.55 (s, 2H), 3.17 (dd, J = |
| | | | | 10.9, 6.3 Hz, 2H), 2.08 (d, J = 4.7 Hz, 2H), 1.92-1.81 |
| | | | | (m, 2H), 1.19-1.14 (m, 3H), 1.05 (d, J = 7.5 Hz, 6H). |
| 592 | **** | *** | 529.3 | 1H NMR (400 MHz, DMSO-d6): δ 9.06 (d, J = 0.5 Hz, |
| | | | | 1H), 9.00 (s, 1H), 8.46 (s, 1H), 7.87 (s, 1H), 7.37-7.27 |
| | | | | (m, 2H), 7.06-6.98 (m, 1H), 6.36 (s, 1H), 6.23 (s, 1H), |
| | | | | 5.49 (d, J = 5.3 Hz, 1H), 4.66 (s, 1H), 4.57 (t, J = 5.7 Hz, |
| | | | | 2H), 3.54 (s, 2H), 3.18 (d, J = 7.4 Hz, 3H), 2.10 (d, J = |
| | | | | 7.3 Hz, 3H), 1.90 (dd, J = 8.5, 3.8 Hz, 3H), 1.07 (d, J = |
| | | | | 5.9 Hz, 6H). |
| 593 | **** | *** | 529.3 | 1H NMR (400 MHz, DMSO-d6): δ 9.06 (s, 1H), 9.00 (s, |
| | | | | 1H), 8.46 (s, 1H), 7.87 (s, 1H), 7.37-7.26 (m, 2H), 7.06- |
| | | | | 6.99 (m, 1H), 6.36 (s, 1H), 6.23 (s, 1H), 5.49 (d, J = 5.3 |
| | | | | Hz, 1H), 4.66 (s, 1H), 4.58 (s, 1H), 4.56 (d, J = 5.3 Hz, |
| | | | | 2H), 3.54 (s, 2H), 3.18 (d, J = 7.3 Hz, 2H), 2.09 (d, J = |
| | | | | 12.3 Hz, 2H), 1.97-1.86 (m, 2H), 1.07 (d, J = 5.9 Hz, 6H). |
| 594 | **** | *** | 524.3 | 1H NMR (400 MHz, DMSO-d6): δ 9.01 (d, J = 0.5 Hz, |
| | | | | 1H), 8.59 (s, 1H), 8.24 (s, 1H), 7.82 (s, 1H), 7.49 (dd, J = |
| | | | | 15.4, 8.5 Hz, 1H), 7.22 (td, J = 10.2, 2.5 Hz, 1H), 7.12- |
| | | | | 7.12 (m, 1H), 5.47 (d, J = 5.3 Hz, 1H), 4.66 (s, 1H), 4.54 |
| | | | | (d, J = 5.3 Hz, 1H), 4.26 (q, J = 7.3 Hz, 2H), 3.61 (s, 2H), |
| | | | | 3.28 (s, 4H), 2.61 (s, 4H), 1.43 (t, J = 7.3 Hz, 3H), 1.06 |
| | | | | (d, J = 9.7 Hz, 6H). |
| 595 | **** | *** | 524.3 | 1H NMR (400 MHz, DMSO-d6): δ 9.01 (s, 1H), 8.59 (s, |
| | | | | 1H), 8.24 (s, 1H), 7.82 (s, 1H), 7.49 (dd, J = 15.4, 8.5 Hz, |
| | | | | 1H), 7.27-7.20 (m, 1H), 7.08 (td, J = 8.5, 2.2 Hz, 1H), |
| | | | | 5.46 (d, J = 5.1 Hz, 1H), 4.66 (s, 1H), 4.54 (d, J = 5.2 Hz, |
| | | | | 1H), 4.26 (q, J = 7.3 Hz, 2H), 3.61 (s, 2H), 3.28 (s, 4H), |
| | | | | 2.61 (s, 4H), 1.43 (t, J = 7.3 Hz, 3H), 1.06 (d, J = 9.7 Hz, 6H). |
| 596 | *** | ** | 529.8 | 1H NMR (400 MHz, DMSO-d6): δ 9.08 (s, 1H), 8.88 (s, |
| | | | | 1H), 8.37 (s, 1H), 7.73 (s, 1H), 7.36-7.26 (m, 2H), 7.06- |
| | | | | 6.98 (m, 1H), 6.35 (s, 1H), 6.22 (s, 1H), 5.49 (d, J = 5.4 |
| | | | | Hz, 1H), 4.75 (s, 1H), 4.58 (d, J = 3.7 Hz, 1H), 4.53 (d, |
| | | | | J = 5.4 Hz, 1H), 3.63 (s, 2H), 3.27 (dd, J = 13.5, 7.9 Hz, |
| | | | | 2H), 2.07 (s, 2H), 1.92-1.81 (m, 2H), 1.07 (d, J = 11.4 |
| | | | | Hz, 6H). |
| 597 | *** | ** | 529.3 | 1H NMR (400 MHz, DMSO-d6): δ 9.08 (s, 1H), 8.88 (s, |
| | | | | 1H), 8.37 (s, 1H), 7.73 (s, 1H), 7.36-7.26 (m, 2H), 7.06- |
| | | | | 6.98 (m, 1H), 6.35 (s, 1H), 6.21 (s, 1H), 5.49 (d, J = 5.4 |
| | | | | Hz, 1H), 4.75 (s, 1H), 4.57 (d, J = 3.5 Hz, 1H), 4.53 (d, |
| | | | | J = 5.4 Hz, 1H), 3.63 (s, 2H), 3.25 (d, J = 13.0 Hz, 2H), |
| | | | | 2.08 (d, J = 4.8 Hz, 2H), 1.86 (d, J = 4.6 Hz, 2H), 1.07 (d, |
| | | | | J = 11.4 Hz, 6H). |
| 598 | *** | ** | 528.3 | 1H NMR (400 MHz, DMSO-d6): δ 9.07 (s, 1H), 8.82 (s, |
| | | | | 1H), 8.33 (s, 1H), 7.71 (s, 1H), 7.48 (dd, J = 15.2, 8.3 Hz, |
| | | | | 1H), 7.21 (td, J = 10.1, 2.5 Hz, 1H), 7.07 (td, J = 8.5, 2.2 |
| | | | | Hz, 1H), 6.33 (s, 1H), 6.20 (s, 1H), 5.49 (d, J = 5.4 Hz, |
| | | | | 1H), 4.74 (s, 1H), 4.52 (d, J = 5.4 Hz, 1H), 3.58 (s, 2H), |
| | | | | 3.41-3.35 (m, 5H), 2.58 (s, 4H), 1.07 (d, J = 9.9 Hz, 6H). |
| 599 | *** | ** | 528.3 | 1H NMR (400 MHz, DMSO-d6): δ 9.07 (s, 1H), 8.81 (s, |
| | | | | 1H), 8.33 (s, 1H), 7.71 (s, 1H), 7.47 (dd, J = 15.4, 8.5 Hz, |
| | | | | 1H), 7.21 (td, J = 10.2, 2.5 Hz, 1H), 7.07 (td, J = 8.5, 2.2 |
| | | | | Hz, 1H), 6.33 (s, 1H), 6.20 (s, 1H), 5.49 (d, J = 5.4 Hz, |
| | | | | 1H), 4.74 (s, 1H), 4.52 (d, J = 5.4 Hz, 1H), 3.58 (s, 2H), |
| | | | | 3.36 (s, 4H), 2.58 (s, 4H), 1.07 (d, J = 9.9 Hz, 6H). |
| 600 | **** | *** | 536.4 | (400 MHz, DMSO-d6): δ 9.01 (s, 1H), 8.58 (s, 1H), 8.22 |
| | | | | (s, 1H), 7.82 (s, 1H), 7.49 (dd, J = 15.4, 8.5 Hz, 1H), 7.23 |
| | | | | (td, J = 10.1, 2.5 Hz, 1H), 7.09 (dd, J = 9.6, 7.4 Hz, 1H), |
| | | | | 5.46 (d, J = 5.3 Hz, 1H), 4.65 (s, 1H), 4.54 (d, J = 5.2 Hz, |
| | | | | 1H), 3.90 (ddd, J = 11.3, 7.5, 3.9 Hz, 1H), 3.61 (s, 2H), |
| | | | | 3.28 (s, 4H), 2.60 (s, 4H), 1.13 (dd, J = 7.7, 4.0 Hz, 2H), |
| | | | | 1.04 (dd, J = 14.1, 8.4 Hz, 8H). |
| 601 | *** | ** | 536.4 | 1H NMR (400 MHz, DMSO-d6): δ 9.01 (s, 1H), 8.58 (s, |
| | | | | 1H), 8.22 (s, 1H), 7.82 (s, 1H), 7.49 (dd, J = 15.4, 8.5 Hz, |
| | | | | 1H), 7.22 (td, J = 10.1, 2.5 Hz, 1H), 7.09 (td, J = 8.4, 2.2 |
| | | | | Hz, 1H), 5.46 (d, J = 5.3 Hz, 1H), 4.66 (s, 1H), 4.54 (d, |
| | | | | J = 5.2 Hz, 1H), 3.90 (ddd, J = 11.3, 7.4, 3.8 Hz, 1H), 3.61 |
| | | | | (s, 2H), 3.27 (d, J = 2.0 Hz, 4H), 2.60 (s, 4H), 1.14 (dt, |
| | | | | J = 7.8, 4.0 Hz, 2H), 1.04 (dd, J = 14.2, 8.4 Hz, 8H). |
| 602 | *** | *** | 528.4 | 1H NMR (400 MHz, DMSO-d6): δ 9.07 (s, 1H), 8.95 (s, |
| | | | | 1H), 8.45 (s, 1H), 7.88 (s, 1H), 7.51 (dd, J = 15.4, 8.5 Hz, |
| | | | | 1H), 7.24 (td, J = 10.2, 2.5 Hz, 1H), 7.10 (td, J = 8.4, 2.1 |
| | | | | Hz, 1H), 6.37 (s, 1H), 6.24 (s, 1H), 5.51 (d, J = 5.2 Hz, |
| | | | | 1H), 4.67 (s, 1H), 4.57 (d, J = 5.1 Hz, 1H), 3.62 (s, 2H), |
| | | | | 3.31 (s, 4H), 2.64 (s, 4H), 1.07 (t, J = 7.6 Hz, 6H). |
| 603 | **** | ** | 528.4 | 1H NMR (400 MHz, DMSO-d6): δ 9.05 (s, 1H), 8.93 (s, |
| | | | | 1H), 8.43 (s, 1H), 7.86 (s, 1H), 7.49 (dd, J = 15.4, 8.5 Hz, |
| | | | | 1H), 7.21 (td, J = 10.2, 2.5 Hz, 1H), 7.08 (td, J = 8.4, 2.1 |
| | | | | Hz, 1H), 6.35 (s, 1H), 6.22 (s, 1H), 5.48 (d, J = 5.2 Hz, |
| | | | | 1H), 4.65 (s, 1H), 4.55 (d, J = 5.0 Hz, 1H), 3.60 (s, 2H), |
| | | | | 3.30-3.25 (m, 4H), 2.61 (s, 4H), 1.06 (d, J = 5.9 Hz, 6H). |
| 604 | *** | ** | 524.4 | 1H NMR (400 MHz, DMSO-d6) δ 9.02 (s, 1H), 8.56 (s, |
| | | | | 1H), 8.24 (s, 1H), 7.99 (s, 1H), 7.54-7.43 (m, 1H), 7.26- |
| | | | | 7.16 (m, 1H), 7.12-7.04 (m, 1H), 5.34 (s, 1H), 4.89 (s, |
| | | | | 1H), 3.96 (s, 3H), 3.60 (s, 2H), 3.30-3.22 (m, 4H), 2.65- |
| | | | | 2.56 (m, 4H), 1.55 (s, 3H), 1.10 (s, 3H), 0.99 (s, 3H). |
| 605 | *** | ** | 550.4 | 1H NMR (400 MHz, DMSO-d6) δ 9.04 (d, J = 0.6 Hz, |
| | | | | 1H), 8.47 (s, 1H), 8.10 (s, 1H), 7.85 (d, J = 0.6 Hz, 1H), |
| | | | | 7.53-7.43 (m, 1H), 7.22 (td, J = 10.1, 2.5 Hz, 1H), 7.08 |
| | | | | (td, J = 8.5, 2.1 Hz, 1H), 5.36 (s, 1H), 5.02 (s, 1H), 3.88 |
| | | | | (tt, J = 7.5, 3.9 Hz, 1H), 3.58 (s, 2H), 3.43-3.29 |
| | | | | (overlapped m, 4H), 2.62-2.53 (overlapped m, 4H), 1.53 |
| | | | | (s, 3H), 1.16-1.06 (m, 5H), 1.05-0.93 (m, 5H). |
| 606 | *** | ** | 510.4 | 1H NMR (400 MHz, DMSO-d6) δ 9.07 (d, J = 0.6 Hz, |
| | | | | 1H), 8.82 (s, 1H), 8.33 (s, 1H), 7.71 (s, 1H), 7.46-7.41 |
| | | | | (m, 1H), 7.36-7.30 (m, 1H), 7.21-7.15 (m, 2H), 6.27 |
| | | | | (d, J = 52.4 Hz, 2H), 5.50 (d, J = 5.4 Hz, 1H), 4.75 (s, |
| | | | | 1H), 4.52 (d, J = 5.3 Hz, 1H), 3.60 (s, 2H), 3.39-3.35 |
| | | | | (m, 4H), 2.61-2.56 (m, 4H), 1.06 (d, J = 9.3 Hz, 6H) |
| 607 | *** | ** | 542.5 | 1H NMR (400 MHz, DMSO-d6) δ 9.08 (d, J = 0.6 Hz, |
| | | | | 1H), 8.82 (s, 1H), 8.33 (s, 1H), 7.88 (d, J = 0.7 Hz, 1H), |
| | | | | 7.51-7.43 (m, 1H), 7.26-7.19 (m, 1H), 7.11-7.03 (m, |
| | | | | 1H), 6.27 (d, J = 52.4 Hz, 2H), 5.38 (s, 1H), 5.01 (s, 1H), |
| | | | | 3.57 (s, 2H), 3.40-3.35 (m, 4H), 2.59-2.55 (m, 4H), |
| | | | | 1.54 (s, 3H), 1.11 (s, 3H), 0.99 (s, 3H) |
| 608 | *** | ** | 508.4 | 1H NMR (400 MHz, DMSO-d6) δ 9.19 (d, J = 0.6 Hz, |
| | | | | 1H), 8.88 (s, 1H), 8.36 (s, 1H), 8.16 (d, J = 0.6 Hz, 1H), |
| | | | | 7.46-7.41 (m, 1H), 7.37-7.29 (m, 1H), 7.22-7.14 (m, |
| | | | | 2H), 6.27 (d, J = 52.3 Hz, 2H), 5.93 (s, 1H), 3.59 (s, 2H), |
| | | | | 3.48-3.43 (m, 4H), 2.59-2.56 (m, 4H), 1.55 (s, 6H). |
| 609 | *** | *** | 498.4 | 1H NMR (400 MHz, DMSO-d6): δ 8.18 (s, 1H), 7.42 (dt, |
| | | | | J = 8.5, 5.5 Hz, 2H), 7.18 (td, J = 10.2, 2.5 Hz, 1H), 7.04 |
| | | | | (td, J = 8.5, 2.3 Hz, 1H), 6.92-6.86 (m, 1H), 6.33 (s, |
| | | | | 2H), 4.57 (d, J = 18.1 Hz, 2H), 3.78 (dd, J = 11.5, 5.7 Hz, |
| | | | | 2H), 3.48 (s, 2H), 3.09 (s, 4H), 2.88 (t, J = 5.6 Hz, 1H), |
| | | | | 2.75 (t, J = 5.6 Hz, 1H), 2.37 (s, 4H), 2.09 (t, J = 9.1 Hz, 3H). |
| 610 | **** | ** | 514.3 | H NMR (400 MHz, DMSO-d6): δ 7.40 (ddd, J = 11.9, |
| | | | | 10.6, 2.6 Hz, 1H), 7.25 (dd, J = 8.2, 1.9 Hz, 1H), 6.89 (dd, |
| | | | | J = 8.0, 2.8 Hz, 1H), 6.33 (s, 2H), 4.57 (d, J = 18.2 Hz, |
| | | | | 2H), 3.81-3.75 (m, 2H), 3.50 (s, 2H), 3.10 (s, 4H), 2.88 |
| | | | | (t, J = 5.5 Hz, 1H), 2.75 (t, J = 5.6 Hz, 1H), 2.38 (s, 4H), |
| | | | | 2.11 (d, J = 11.7 Hz, 3H). |
| 611 | **** | ** | 515.3 | 1H NMR (400 MHz, DMSO-d6): δ 7.45 (dd, J = 11.2, 8.1 |
| | | | | Hz, 1H), 7.30-7.21 (m, 2H), 7.02-6.95 (m, 1H), 6.92 |
| | | | | (dd, J = 8.0, 3.0 Hz, 1H), 6.35 (s, 2H), 4.51 (s, 2H), 4.48- |
| | | | | 4.42 (m, 1H), 3.73 (t, J = 5.8 Hz, 2H), 3.65 (s, 3H), 3.40 |
| | | | | (t, J = 12.1 Hz, 2H), 2.98 (t, J = 9.7 Hz, 2H), 2.82 (t, J = |
| | | | | 5.7 Hz, 2H). 1.88 (s, 2H), 1.66-1.54 (m, 2H). |
| 612 | *** | ** | 528.3 | 1H-NMR (400 MHz, DMSO-d6) δ 9.09 (s, 1H), 8.90 (s, |
| | | | | 1H), 8.43 (s, 1H), 7.95 (t, J = 58.7 Hz, 1H), 7.72 (s, 1H), |
| | | | | 7.43 (td, J = 7.7, 1.8 Hz, 1H), 7.37-7.29 (m, 1H), 7.21- |
| | | | | 7.14 (m, 2H), 5.51 (d, J = 5.4 Hz, 1H), 4.74 (s, 1H), 4.52 |
| | | | | (d, J = 5.4 Hz, 1H), 3.60 (s, 2H), 3.39-3.34 (overlapping |
| | | | | m, 4H), 2.60-2.53 (m, 4), 1.07 (s, 3H), 1.06 (s, 3H). |
| 613 | *** | ** | 528.3 | 1H-NMR (400 MHz, DMSO-d6) δ 9.09 (s, 1H), 8.90 (s, |
| | | | | 1H), 8.43 (s, 1H), 7.95 (t, J = 58.7 Hz, 1H), 7.72 (s, 1H), |
| | | | | 7.47-7.39 (m, 1H), 7.36-7.28 (m, 1H), 7.21-7.13 (m, |
| | | | | 2H), 5.51 (d, J = 5.4 Hz, 1H), 4.74 (s, 1H), 4.52 (d, J = |
| | | | | 5.3 Hz, 1H), 3.60 (s, 2H), 3.40-3.34 (overlapping m, |
| | | | | 4H), 2.61-2.53 (m, 4H), 1.07 (s, 3H), 1.05 (s, 3H). |
| 614 | *** | ** | 511.4 | 1H NMR (400 MHz, DMSO-d6) δ 9.10-9.04 (m, 1H), |
| | | | | 8.51 (s, 1H), 8.21-8.12 (m, 1H), 7.85-7.76 (m, 1H), |
| | | | | 7.38-7.22 (m, 2H), 7.07-6.95 (m, 1H), 5.38 (s, 1H), |
| | | | | 4.65-4.47 (m, 1H), 3.99-3.92 (m, 3H), 3.74-3.61 (m, |
| | | | | 3H), 3.60-3.51 (m, 1H), 3.29-3.21 (overlapping m, |
| | | | | 2H), 3.21-3.20 (overlapping m, 3H), 2.16-2.00 (m, |
| | | | | 2H), 1.92-1.76 (m, 2H), 1.45 (s, 3H); |
| 615 | *** | ** | 511.4 | 1H NMR (400 MHz, DMSO-d6) δ 9.06 (d, J = 0.6 Hz, |
| | | | | 1H), 8.50 (s, 1H), 8.16 (s, 1H), 7.82 (d, J = 0.6 Hz, 1H), |
| | | | | 7.41-7.21 (m, 2H), 7.08-6.97 (m, 1H), 5.37 (s, 1H), |
| | | | | 4.62-4.51 (m, 1H), 3.95 (s, 3H), 3.73-3.60 (m, 3H), |
| | | | | 3.60-3.51 (m, 1H), 3.30-3.21 (m, 2H), 3.20 (s, 3H), |
| | | | | 2.17-2.03 (m, 2H), 1.93-1.76 (m, 2H), 1.45 (s, 3H). |
| 616 | *** | ** | 545.4 | 1H NMR (400 MHz, DMSO-d6) δ 9.22 (s, 1H), 9.03 (s, |
| | | | | 1H), 8.51 (s, 1H), 8.18 (s, 1H), 7.97 (t, J = 58.6 Hz, 1H), |
| | | | | 7.36-7.26 (m, 2H), 7.02 (s, 1H), 5.92 (s, 1H), 4.60 (s, |
| | | | | 1H), 3.71 (dd, J = 9.9, 5.9 Hz, 2H), 3.34 (t, J = 9.3 Hz, |
| | | | | 4H), 2.07 (dd, J = 8.9, 3.5 Hz, 2H), 1.88-1.77 (m, 2H), |
| | | | | 1.56 (s, 6H). |
| 617 | *** | ** | 524.3 | 1H NMR (400 MHz, DMSO-d6) δ 9.04 (s, 1H), 8.45 (s, |
| | | | | 1H), 8.12 (s, 1H), 7.86 (s, 1H), 7.48 (dd, J = 15.5, 8.5 Hz, |
| | | | | 1H), 7.22 (td, J = 10.0, 2.5 Hz, 1H), 7.08 (dt, J = 8.5, 4.2 |
| | | | | Hz, 1H), 5.37 (s, 1H), 5.03 (s, 1H), 3.95 (s, 3H), 3.58 (s, |
| | | | | 2H), 3.34-3.29 (over lapping m, 2H), 2.59-2.55 (m, |
| | | | | 4H), 2.54 (s, 1H) 1.54 (s, 3H), 1.12 (s, 3H), 0.99 (s, 3H). |
| | | | | The last proton missing is overlapping with one of the |
| | | | | present peak. |
| 618 | *** | ** | 526.3 | 1H-NMR (400 MHz, DMSO-d6) δ 9.20 (d, J = 0.6 Hz, |
| | | | | 1H), 8.96 (s, 1H), 8.46 (s, 1H), 8.16 (d, J = 0.6 Hz, 1H), |
| | | | | 7.95 (t, J = 58.6 Hz, 1H), 7.43 (td, J = 7.7, 1.8 Hz, 1H), |
| | | | | 7.37-7.28 (m, 1H), 7.22-7.14 (m, 2H), 5.91 (s, 1H), |
| | | | | 3.59 (s, 2H), 3.48-3.41 (m, 4H), 2.58-2.53 (m, 4H), |
| | | | | 1.55 (s, 6H). |
| 619 | *** | ** | 534.4 | 1H NMR (400 MHz, DMSO-d6) δ 9.14 (d, J = 0.6 Hz, |
| | | | | 1H), 8.53 (s, 1H), 8.17-8.11 (m, 2H), 7.53-7.42 (m, |
| | | | | 1H), 7.27-7.17 (m, 1H), 7.08 (td, J = 8.5, 2.3 Hz, 1H), |
| | | | | 5.96 (s, 1H), 3.89 (tt, J = 7.5, 3.9 Hz, 1H), 3.57 (s, 2H), |
| | | | | 3.48-3.41 (m, 4H), 2.59-2.53 (overlapped m, 4H), 1.54 |
| | | | | (s, 6H), 1.17-1.11 (m, 2H), 1.06-1.00 (m, 2H). |
| 620 | *** | *** | 544.3 | 1H NMR (400 MHz, DMSO-d6) δ 9.15 (d, J = 0.7 Hz, |
| | | | | 1H), 9.01 (s, 1H), 8.51 (s, 1H), 8.38 (d, J = 0.7 Hz, 1H), |
| | | | | 7.96 (t, J = 58.6 Hz, 1H), 7.51-7.41 (m, 1H), 7.22 (ddd, |
| | | | | J = 10.2, 9.6, 2.6 Hz, 1H), 7.08 (td, J = 8.3, 1.9 Hz, 1H), |
| | | | | 5.85 (s, 1H), 3.58 (s, 2H), 3.48-3.37 (m, 4H), 2.61- |
| | | | | 2.55 (overlapped m, 4H), 1.56 (s, 6H). |
| 621 | *** | ** | 527.4 | 1H NMR (400 MHz, DMSO-d6) δ 9.20 (d, J = 0.6 Hz, |
| | | | | 1H), 8.94 (s, 1H), 8.40 (s, 1H), 8.18 (d, J = 0.6 Hz, 1H), |
| | | | | 7.36-7.26 (m, 2H), 7.06-6.98 (m, 1H), 6.29 (d, J = |
| | | | | 52.2 Hz, 2H), 5.93 (s, 1H), 4.63-4.55 (m, 1H), 3.77- |
| | | | | 3.65 (m, 2H), 3.37-3.29 (m, 2H, overlapped), 2.12- |
| | | | | 2.05 (m, 2H), 1.90-1.80 (m, 2H), 1.55 (s, 6H). |
| 622 | *** | ** | 524.4 | 1H NMR (400 MHz, DMSO-d6) δ 9.08 (d, J = 0.6 Hz, |
| | | | | 1H), 8.82 (s, 1H), 8.33 (s, 1H), 7.88 (d, J = 0.6 Hz, 1H), |
| | | | | 7.47-7.41 (m, 1H), 7.36-7.30 (m, 1H), 7.21-7.15 (m, |
| | | | | 2H), 6.27 (d, J = 52.4 Hz, 2H), 5.38 (s, 1H), 5.01 (s, 1H), |
| | | | | 3.60 (s, 2H), 3.39-3.36 (m, 4H), 2.61-2.57 (m, 4H), |
| | | | | 1.54 (s, 3H), 1.11 (s, 3H), 0.99 (s, 3H) |
| 623 | *** | *** | 447.4 | 1H NMR (400 MHz, ACN-d3) δ 8.19 (d, J = 2.2 Hz, |
| | | | | 1H), 8.09 (d, J = 5.0 Hz, 1H), 7.31-7.22 (m, 2H), 7.19- |
| | | | | 7.05 (m, 2H), 6.31 (s, 1H), 4.60 (m, 2H), 3.91 (s, 3H), |
| | | | | 3.83 (t, J = 5.9 Hz, 1H), 3.77 (t, J = 6.0 Hz, 1H), 3.25- |
| | | | | 3.18 (m, 2H), 3.15-3.08 (m, 2H), 2.88 (t, J = 6.0 Hz, |
| | | | | 1H), 2.77 (t, J = 5.9 Hz, 1H), 2.59-2.48 (m, 4H), 2.12 (s, |
| | | | | 1.5H), 2.11 (s, 1.5H). |
| 624 | *** | ** | 522.4 | 1H-NMR (400 MHz, DMSO-d6) δ 9.15 (s, 1H), 8.55 (s, |
| | | | | 1H), 8.39 (s, 1H), 8.18 (s, 1H), 8.15 (s, 1H), 7.48 (dd, J = |
| | | | | 15.5, 8.4 Hz, 1H), 7.23 (td, J = 10.0, 2.5 Hz, 1H), 7.09 |
| | | | | (td, J = 8.4, 2.3 Hz, 1H), 5.97 (s, 1H), 4.26 (q, J = 7.2 Hz, |
| | | | | 2H), 3.58 (s, 2H), 3.45 (s, 4H), 2.60-2.55 (m, 4H), 1.55 |
| | | | | (s, 6H), 1.44 (t, J = 7.3 Hz, 3H). |
| 625 | *** | ** | 535.3 | 1H NMR (400 MHz, DMSO-d6) δ 9.15 (d, J = 0.6 Hz, |
| | | | | 1H), 8.60 (s, 1H), 8.17 (d, J = 0.5 Hz, 1H), 8.16 (d, J = |
| | | | | 0.6 Hz, 1H), 7.36-7.26 (m, 2H), 7.06-6.98 (m, 1H), |
| | | | | 5.97 (s, 1H), 4.65-4.54 (m, 1H), 3.92 (tt, J = 7.5, 3.9 Hz, |
| | | | | 1H), 3.78-3.65 (m, 2H), 3.41-3.27 (overlapped m, |
| | | | | 2H), 2.13-2.01 (m, 2H), 1.88-1.75 (m, 2H), 1.55 (s, |
| | | | | 6H), 1.19-1.12 (m, 2H), 1.07-0.99 (m, 2H). |
| 626 | **** | ** | 511.4 | 1H NMR (400 MHz, DMSO-d6): δ 7.45 (dd, J = 11.1, 8.1 |
| | | | | Hz, 1H), 7.32-7.20 (m, 2H), 6.94 (dt, J = 23.7, 11.7 Hz, |
| | | | | 3H), 6.36 (s, 2H), 6.16 (d, J = 17.2 Hz, 1H), 5.73 (t, J = |
| | | | | 10.6 Hz, 1H), 4.69 (d, J = 34.2 Hz, 2H), 4.45 (s, 1H), 3.90 |
| | | | | (d, J = 16.2 Hz, 2H), 3.41 (s, 3H), 3.05-2.93 (m, 2H), |
| | | | | 2.84 (d, J = 24.2 Hz, 3H), 1.88 (s, 2H), 1.75 (s, 1H), 1.60 |
| | | | | (d, J = 8.8 Hz, 2H) |
| 627 | *** | ** | 563.4 | 1H NMR (400 MHz, DMSO-d6): δ 7.45 (dd, J = 11.2, 8.1 |
| | | | | Hz, 1H), 7.25 (ddt, J = 7.2, 5.5, 3.6 Hz, 2H), 7.02-6.95 |
| | | | | (m, 1H), 6.93 (dd, J = 8.1, 3.0 Hz, 1H), 6.37 (s, 2H), 4.45 |
| | | | | (dd, J = 8.2, 4.1 Hz, 1H), 4.41 (s, 2H), 3.67 (t, J = 5.8 Hz, |
| | | | | 2H), 3.49 (dd, J = 13.6, 6.8 Hz, 1H), 3.42 (dd, J = 18.4, |
| | | | | 10.0 Hz, 3H), 3.04-2.95 (m, 2H), 2.87 (t, J = 5.7 Hz, |
| | | | | 2H), 1.90 (d, J = 9.9 Hz, 2H), 1.61 (dd, J = 11.2, 6.7 Hz, |
| | | | | 2H), 1.25 (d, J = 6.8 Hz, 6H) |
| 628 | *** | ** | 529.4 | 1H NMR (400 MHz, DMSO-d6): δ 7.47 (m, 1H), 7.29- |
| | | | | 7.21 (m, 2H), 7.00-6.91 (m, 2H), 6.36 (s, 2H), 4.57 (s, |
| | | | | 2H), 4.46 (m, 1H), 4.22 (d, J = 14.4 Hz, 2H), 3.81 (m, |
| | | | | 1H), 3.74 (m, 1H), 3.40 (bs, 2H), 3.29 (s, 2H-Merged |
| | | | | with DMSO water peak, 3.04-2.66 (m, 4H), 1.90 (m, |
| | | | | 2H), 1.63 (m, 2H) |
| 629 | **** | ** | 528.3 | 1H NMR (400 MHz, DMSO-d6): δ 7.44 (dd, J = 11.2, 8.1 |
| | | | | Hz, 1H), 7.25 (ddd, J = 9.3, 8.3, 4.1 Hz, 2H), 7.04-6.88 |
| | | | | (m, 2H), 6.34 (s, 2H), 4.51-4.39 (m, 1H), 4.28 (s, 2H), |
| | | | | 3.49 (t, J = 5.7 Hz, 2H), 3.47-3.37 (m, 2H), 3.05-2.91 |
| | | | | (m, 2H), 2.85 (t, J = 5.5 Hz, 2H), 2.80 (s, 6H), 1.89 (d, J = |
| | | | | 11.5 Hz, 2H), 1.68-1.54 (m, 2H). |
| 630 | ** | * | 482.3 | 1H NMR (400 MHz, DMSO-d6): δ 8.37 (d, J = 2.2 Hz, |
| | | | | 1H), 8.07 (d, J = 2.9 Hz, 1H), 7.51 (dd, J = 14.8, 8.2 Hz, |
| | | | | 1H), 7.38 (td, J = 9.7, 2.4 Hz, 1H), 7.20 (td, J = 8.4, 2.2 |
| | | | | Hz, 1H), 4.63 (d, J = 16.5 Hz, 2H), 3.91 (d, J = 1.6 Hz, |
| | | | | 3H), 3.83 (d, J = 13.8 Hz, 2H), 3.83-3.74 (m, 2H), 3.45 |
| | | | | (s, 2H), 3.09 (s, 2H), 3.04 (s, 2H), 2.90 (t, J = 5.7 Hz, |
| | | | | 1H), 2.76 (t, J = 5.6 Hz, 1H), 2.12 (d, J = 5.0 Hz, 3H). |
| 631 | *** | ** | 539.4 | 1H NMR (400 MHz, DMSO-d6) δ 9.06 (s, 1H), 8.54 (s, |
| | | | | 1H), 8.16 (s, 1H), 7.88 (s, 1H), 7.37-7.26 (m, 2H), 7.05- |
| | | | | 7.00 (m, 1H), 5.37 (s, 1H), 5.04 (s, 1H), 4.62-4.54 (m, |
| | | | | 1H), 4.26 (q, J = 7.2 Hz, 2H), 3.70-3.61 (m, 2H), 3.29- |
| | | | | 3.24 (m, 2H), 2.14-2.05 (m, 2H), 1.86-1.83 (m, 2H), |
| | | | | 1.54 (s, 3H), 1.44 (t, J = 7.2 Hz, 3H), 1.12 (s, 3H), 0.99 (s, 3H). |
| 632 | *** | ** | 551.4 | 1H NMR (400 MHz, DMSO-d6) δ 9.05 (d, J = 0.7 Hz, |
| | | | | 1H), 8.54 (s, 1H), 8.13 (d, J = 0.6 Hz, 1H), 7.87 (d, J = |
| | | | | 0.7 Hz, 1H), 7.37-7.26 (m, 2H), 7.07-6.97 (m, 1H), |
| | | | | 5.36 (s, 1H), 5.03 (s, 1H), 4.67-4.51 (m, 1H), 3.91 (tt, |
| | | | | J = 7.5, 3.9 Hz, 1H), 3.73-3.57 (m, 2H), 3.30-3.21 (m, |
| | | | | 2H), 2.14-2.00 (m, 2H), 1.90-1.73 (m, 2H), 1.54 (s, |
| | | | | 3H), 1.18-1.08 (overlapped m, 5H), 1.06-0.97 |
| | | | | (overlapped m, 5H). |
| 633 | **** | ** | 560.4 | 1H NMR (400 MHz, DMSO-d6) δ 9.08 (d, J = 0.7 Hz, |
| | | | | 1H), 9.01 (s, 1H), 8.53 (s, 1H), 8.05 (overlapped d, J = |
| | | | | 0.7 Hz, 1H), 7.97 (t, J = 58.6 Hz, 1H), 7.54-7.44 (m, |
| | | | | 1H), 7.22 (ddd, J = 10.2, 9.6, 2.6 Hz, 1H), 7.08 (td, J = |
| | | | | 8.4, 2.1 Hz, 1H), 5.34 (s, 1H), 4.85 (s, 1H), 3.59 (s, 2H), |
| | | | | 3.31-3.23 (m, 4H), 2.65-2.55 (m, 4H), 1.56 (s, 3H), |
| | | | | 1.09 (s, 3H), 1.01 (s, 3H). |
| 634 | *** | ** | 542.4 | 1H-NMR (400 MHz, DMSO-d6) δ 9.06 (d, J = 0.6 Hz, |
| | | | | 1H), 8.86 (s, 1H), 8.39 (s, 1H), 8.07-7.74 (overlapping |
| | | | | signals, 2H), 7.39 (td, J = 7.8, 1.8 Hz, 1H), 7.32-7.25 |
| | | | | (m, 1H), 7.17-7.10 (m, 2H), 5.34 (s, 1H), 4.96 (s, 1H), |
| | | | | 3.56 (s, 2H), 3.36-3.32 (overlapping m, 4H), 2.56-2.49 |
| | | | | (m, 4H), 1.50 (s, 3H), 1.07 (s, 3H), 0.95 (s, 3H). |
| 635 | *** | ** | 561.5 | 1H NMR (400 MHz, DMSO-d6) δ 9.12 (s, 1H), 8.97 (s, |
| | | | | 1H), 8.47 (s, 1H), 7.96 (t, J = 58.6 Hz, 1H), 7.91 (s, 1H) |
| | | | | 7.36-7.26 (m, 2H), 7.06-6.98 (m, 1H), 5.38 (s, 1H), |
| | | | | 5.01 (s, 1H), 4.60-4.57 (m, 1H), 3.66-3.63 (m, 2H), |
| | | | | 3.33-3.24 (m, 2H), 2.09-2.06 (m, 2H), 1.85-1.82 (m, |
| | | | | 2 Hz), 1.55 (s, 3H), 1.12 (s, 3H), 1.01 (s, 3H). |
| 636 | *** | ** | 543.5 | 1H NMR (400 MHz, DMSO-d6) δ 9.09 (d, J = 0.6 Hz, |
| | | | | 1H), 8.88 (s, 1H), 8.36 (s, 1H), 7.90 (d, J = 0.6 Hz, 1H), |
| | | | | 7.35-7.27 (m, 2H), 7.05-6.98 (m, 1H), 6.28 (d, J = |
| | | | | 52.4 Hz, 2H), 5.37 (s, 1H), 5.02 (s, 1H), 4.62-4.54 (m, |
| | | | | 1H), 3.69-3.61 (m, 2H), 3.30-3.22 (m, 2H), 2.12- |
| | | | | 2.04 (m, 2H), 1.90-1.81 (m, 2H), 1.55 (s, 3H), 1.12 (s, |
| | | | | 3H), 1.00 (s, 3H). |
| 637 | *** | ** | 530.3 | 1H NMR (400 MHz, DMSO-d6): δ 7.57 (d, J = 2.1 Hz, |
| | | | | 1H), 7.49 (d, J = 8.3 Hz, 1H), 7.47-7.37 (m, 2H), 6.94- |
| | | | | 6.88 (m, 1H), 6.34 (s, 2H), 4.58 (d, J = 18.1 Hz, 2H), 3.78 |
| | | | | (q, J = 5.7 Hz, 2H), 3.54 (s, 2H), 3.12 (s, 4H), 2.88 (t, J = |
| | | | | 5.6 Hz, 1H), 2.75 (t, J = 5.7 Hz, 1H), 2.42 (s, 4H), 2.15- |
| | | | | 2.08 (m, 3H). |
| 638 | **** | ** | 511.3 | 1H NMR (400 MHz, DMSO-d6): δ 7.86 (td, J = 8.6, 6.8 |
| | | | | Hz, 1H), 7.49-7.35 (m, 2H), 7.23 (td, J = 8.4, 2.3 Hz, |
| | | | | 1H), 6.90 (dt, J = 8.0, 2.7 Hz, 1H), 6.33 (s, 2H), 4.58 (d, |
| | | | | J = 18.1 Hz, 2H), 3.78 (q, J = 5.7 Hz, 2H), 3.69-3.53 (m, |
| | | | | 2H), 3.25 (t, J = 11.0 Hz, 1H), 2.89 (t, J = 5.6 Hz, 1H), |
| | | | | 2.84-2.72 (m, 3H), 2.11 (d, J = 11.5 Hz, 3H), 1.75 (d, |
| | | | | J = 11.2 Hz, 2H), 1.50 (dd, J = 21.2, 11.2 Hz, 2H). |
| 639 | *** | *** | 543.5 | 1H NMR (400 MHz, DMSO-d6): δ 7.52-7.47 (m, 1H), |
| | | | | 7.29-7.21 (m, 2H), 7.09-6.95 (m, 2H), 6.54 (bs, 2H), 5.51 |
| | | | | (bs, 1H), 5.41 (bs, 1H), 4.60 (bs, 1H), 4.47-4.43 (m, 1H), |
| | | | | 4.23-4.22 (bs, 1H), 3.83 (bs, 1H), 2.98 (m, 2H), 2.84 (bs, |
| | | | | 2H), 1.90-1.89 (m, 2H), 1.64-1.58 (m, 2H), 1.20 (s, 6H) |
| 640 | *** | ** | 497.4 | 1H NMR (400 MHz, DMSO-d6) δ 9.11-9.03 (m, 1H), |
| | | | | 8.50 (s, 1H), 8.21-8.11 (m, 1H), 7.55 (s, 1H), 7.39- |
| | | | | 7.23 (m, 2H), 7.07-6.97 (m, 1H), 4.85 (t, J = 6.0 Hz, |
| | | | | 1H), 4.63-4.52 (m, 1H), 4.41 (dd, J = 6.5, 3.8 Hz, 1H), |
| | | | | 3.96 (s, 3H), 3.80-3.69 (m, 1H), 3.71-3.58 (m, 3H), |
| | | | | 3.32 (overlapping s, 3H), 3.30-3.21 (overlapping m, |
| | | | | 2H), 2.17-2.01 (m, 2H), 1.92-1.74 (m, 2H) |
| 641 | *** | ** | 497.4 | 1H NMR (400 MHz, DMSO-d6) δ 9.18-9.05 (m, 1H), |
| | | | | 8.58-8.39 (m, 1H), 8.22-8.07 (m, 1H), 7.60-7.49 (m, |
| | | | | 1H), 7.39-7.18 (m, 2H), 7.02 (t, J = 8.6 Hz, 1H), 4.91- |
| | | | | 4.81 (m, 1H), 4.63-4.52 (m, 1H), 4.45-4.36 (m, 1H), |
| | | | | 4.00-3.93 (m, 3H), 3.78-3.69 (m, 1H), 3.69-3.57 (m, |
| | | | | 3H), 3.32-3.31 (overlapping m, 3H), 3.30-3.22 |
| | | | | (overlapping m, 2H), 2.15-2.00 (m, 2H), 1.91-1.75 (m, 2H) |
| 642 | *** | ** | 497.4 | 1H NMR (400 MHz, DMSO-d6) δ 9.06-9.01 (m, 1H), |
| | | | | 8.53-8.48 (m, 1H), 8.18-8.12 (m, 1H), 7.71-7.65 (m, |
| | | | | 1H), 7.37-7.25 (m, 2H), 7.08-6.96 (m, 1H), 5.71- |
| | | | | 5.60 (m, 1H), 4.92-4.82 (m, 1H), 4.61-4.52 (m, 1H), |
| | | | | 4.00-3.91 (m, 3H), 3.78-3.69 (m, 1H), 3.69-3.59 (m, |
| | | | | 2H), 3.60-3.51 (m, 1H), 3.29-3.26 (overlapping m, |
| | | | | 5H), 2.17-1.99 (m, 2H), 1.92-1.76 (m, 2H); |
| 643 | **** | ** | 497.4 | 1H NMR (400 MHz, DMSO-d6) δ 9.07-9.02 (m, 1H), |
| | | | | 8.53-8.46 (m, 1H), 8.18-8.13 (m, 1H), 7.70-7.65 (m, |
| | | | | 1H), 7.37-7.25 (m, 2H), 7.06-6.97 (m, 1H), 5.70- |
| | | | | 5.61 (m, 1H), 4.94-4.82 (m, 1H), 4.65-4.50 (m, 1H), |
| | | | | 3.99-3.90 (m, 3H), 3.80-3.69 (m, 1H), 3.69-3.59 (m, |
| | | | | 2H), 3.60-3.50 (m, 1H), 3.30-3.19 (overlapping m, |
| | | | | 5H), 2.16-2.00 (m, 2H), 1.93-1.75 (m, 2H); |
| 644 | **** | *** | 497.4 | 1H NMR (400 MHz, DMSO-d6) δ 9.07-8.99 (m, 1H), |
| | | | | 8.65-8.55 (m, 1H), 8.30-8.20 (m, 1H), 7.82-7.77 (m, |
| | | | | 1H), 7.38-7.23 (m, 2H), 7.07-6.96 (m, 1H), 5.66 (t, J = |
| | | | | 5.2 Hz, 1H), 4.94-4.84 (m, 1H), 4.62-4.51 (m, 1H), |
| | | | | 4.03-3.91 (m, 3H), 3.77-3.67 (m, 1H), 3.62-3.47 (m, |
| | | | | 3H), 3.29-3.25 (m, 3H), 3.23-3.12 (m, 2H), 2.18- |
| | | | | 2.03 (m, 2H), 1.97-1.81 (m, 2H); |
| 645 | *** | ** | 497.4 | 1H NMR (400 MHz, DMSO-d6) δ 9.04 (s, 1H), 8.61 (s, |
| | | | | 1H), 8.27 (s, 1H), 7.94 (s, 1H), 7.38-7.26 (m, 2H), 7.08- |
| | | | | 6.97 (m, 1H), 5.21 (s, 1H), 4.64-4.54 (m, 2H), 3.97 (s, |
| | | | | 3H), 3.68-3.50 (m, 4H), 3.22-3.11 (m, 2H), 2.17- |
| | | | | 2.04 (m, 2H), 1.96-1.81 (m, 2H), 1.45 (s, 3H) |
| 646 | *** | ** | 429.5 | 1H NMR (400 MHz, DMSO-d6) δ 8.41-8.33 (m, 1H), |
| | | | | 8.11-8.03 (m, 1H), 7.39-7.29 (m, 2H), 7.28-7.16 (m, |
| | | | | 3H), 6.39 (s, 1H), 4.69-4.53 (m, 2H), 3.99-3.88 (m, |
| | | | | 3H), 3.85-3.70 (m, 2H), 3.22-3.11 (m, 2H), 3.10- |
| | | | | 3.00 (m, 2H), 2.94-2.84 (m, 1H), 2.81-2.70 (m, 1H), |
| | | | | 2.65-2.58 (m, 2H), 2.55-2.51 (m, 2H, overlapping), |
| | | | | 2.15-2.08 (m, 3H). |
| 647 | **** | ** | 510.4 | 1H NMR (400 MHz, DMSO-d6) δ 9.07 (d, J = 0.6 Hz, |
| | | | | 1H), 8.82 (s, 1H), 8.33 (s, 1H), 7.71 (s, 1H), 7.47-7.40 |
| | | | | (m, 1H), 7.36-7.30 (m, 1H), 7.21-7.15 (m, 2H), 6.27 |
| | | | | (d, J = 52.4 Hz, 2H), 5.50 (d, J = 5.4 Hz, 1H), 4.75 (s, |
| | | | | 1H), 4.52 (d, J = 5.3 Hz, 1H), 3.60 (s, 2H), 3.39-3.35 |
| | | | | (m, 4H), 2.61-2.56 (m, 4H), 1.06 (d, J = 9.2 Hz, 6H) |
| 648 | *** | ** | 510.5 | 1H NMR (400 MHz, DMSO-d6) δ 9.07 (d, J = 0.6 Hz, |
| | | | | 1H), 8.82 (s, 1H), 8.33 (s, 1H), 7.71 (s, 1H), 7.46-7.41 |
| | | | | (m, 1H), 7.36-7.30 (m, 1H), 7.22-7.15 (m, 2H), 6.27 |
| | | | | (d, J = 52.4 Hz, 2H), 5.50 (d, J = 5.4 Hz, 1H), 4.75 (s, |
| | | | | 1H), 4.52 (d, J = 5.3 Hz, 1H), 3.60 (s, 2H), 3.39-3.34 |
| | | | | (m, 4H), 2.62-2.55 (m, 4H), 1.06 (d, J = 9.2 Hz, 6H) |
| 649 | *** | ** | 542.5 | 1H NMR (400 MHz, DMSO-d6) δ 9.08 (d, J = 0.6 Hz, |
| | | | | 1H), 8.82 (s, 1H), 8.33 (s, 1H), 7.88 (d, J = 0.6 Hz, 1H), |
| | | | | 7.51-7.43 (m, 1H), 7.25-7.18 (m, 1H), 7.11-7.04 (m, |
| | | | | 1H), 6.27 (d, J = 52.4 Hz, 2H), 5.37 (s, 1H), 5.01 (s, 1H), |
| | | | | 3.57 (s, 2H), 3.38-3.35 (m, 4H), 2.59-2.55 (m, 4H), |
| | | | | 1.54 (s, 3H), 1.11 (s, 3H), 0.99 (s, 3H) |
| 650 | *** | ** | 542.5 | 1H NMR (400 MHz, DMSO-d6) δ 9.08 (d, J = 0.7 Hz, |
| | | | | 1H), 8.82 (s, 1H), 8.33 (s, 1H), 7.88 (d, J = 0.7 Hz, 1H), |
| | | | | 7.51-7.43 (m, 1H), 7.25-7.18 (m, 1H), 7.11-7.05 (m, |
| | | | | 1H), 6.27 (d, J = 52.4 Hz, 2H), 5.37 (s, 1H), 5.01 (s, 1H), |
| | | | | 3.57 (s, 2H), 3.37 (m, 4H), 2.57 (m, 4H), 1.54 (s, 3H), |
| | | | | 1.11 (s, 3H), 0.99 (s, 3H) |
| 651 | *** | ** | 523.5 | 1H NMR (400 MHz, DMSO-d6) δ 9.15 (s, 1H), 8.60 (s, |
| | | | | 1H), 8.20 (s, 1H), 8.16 (s, 1H), 7.36-7.26 (m, 2H), 7.05- |
| | | | | 6.98 (m, 1H), 5.97 (s, 1H), 4.59 (m, 1H), 4.27 (q, J = 7.2 |
| | | | | Hz, 2H), 3.76-3.68 (m, 2H), 3.38-3.34 (m, 2H), 2.13- |
| | | | | 2.04 (m, 2H), 1.87-1.82 (m, 2H), 1.55 (s, 6H), 1.43 (t, J = |
| | | | | 7.2 Hz, 3H). |
| 652 | **** | ** | 511.4 | 1H NMR (400 MHz, DMSO-d6) δ 9.04 (d, J = 0.6 Hz, |
| | | | | 1H), 8.61 (s, 1H), 8.26 (s, 1H), 7.93 (d, J = 0.6 Hz, 1H), |
| | | | | 7.37-7.26 (m, 2H), 7.06-6.98 (m, 1H), 5.39 (s, 1H), |
| | | | | 4.63-4.53 (m, 1H), 3.97 (s, 3H), 3.65 (d, J = 9.4 Hz, |
| | | | | 1H), 3.56 (overlapping d, J = 9.4 Hz, 1H), 3.55-3.50 |
| | | | | (overlapping m, 2H), 3.20 (overlapping s, 3H), 3.19- |
| | | | | 3.13 (overlapping m, 2H), 2.17-2.05 (m, 2H), 1.96- |
| | | | | 1.82 (m, 2H), 1.46 (s, 3H) |
| 653 | *** | ** | 550.46 | 1H NMR (400 MHz, DMSO-d6) δ 9.02 (s, 1H), 8.58 (s, |
| | | | | 1H), 8.22 (s, 1H), 7.99 (s, 1H), 7.55-7.43 (m, 1H), 7.27- |
| | | | | 7.16 (m, 1H), 7.12-7.03 (m, 1H), 5.34 (s, 1H), 4.88 (s, |
| | | | | 1H), 3.90 (tt, J = 7.5, 3.9 Hz, 1H), 3.60 (s, 2H), 3.30- |
| | | | | 3.20 (m, 4H), 2.64-2.56 (m, 4H), 1.55 (s, 3H), 1.17- |
| | | | | 1.10 (m, 2H), 1.09 (s, 3H), 1.06-1.00 (m, 2H), 0.99 (s, 3H). |
| 654 | *** | ** | 539.4 | 1H NMR (400 MHz, DMSO-d6) δ 9.06 (s, 1H), 8.54 (s, |
| | | | | 1H), 8.16 (s, 1H), 7.88 (s, 1H), 7.37-7.26 (m, 2H), 7.05- |
| | | | | 7.00 (m, 1H), 5.37 (s, 1H), 5.04 (s, 1H), 4.62-4.54 (m, |
| | | | | 1H), 4.26 (q, J = 7.2 Hz, 2H), 3.70-3.61 (m, 2H), 3.29- |
| | | | | 3.24 (m, 2H), 2.14-2.05 (m, 2H), 1.86-1.83 (m, 2H), |
| | | | | 1.54 (s, 3H), 1.44 (t, J = 7.2 Hz, 3H), 1.12 (s, 3H), 0.99 (s, 3H). |
| 655 | *** | ** | 561.5 | 1H NMR (400 MHz, ACN-d3) δ 9.07 (s, 1H), 8.69 (s, |
| | | | | 1H), 8.40 (s, 1H), 7.85 (s, 1H), 7.47 (t, J = 59.5 Hz, 1H), |
| | | | | 7.20-7.12 (m, 1H), 7.04-6.96 (m, 1H), 6.94-6.87 (m, |
| | | | | 1H), 5.45 (s, 1H), 4.54-4.51 (m, 2H), 4.20 (s, 1H), 3.74- |
| | | | | 3.65 (m, 2H), 3.34-3.25 (m, 2H), 2.11-2.04 (m, 2H), |
| | | | | 1.91-1.83 (m, 2H), 1.61 (s, 3H), 1.18 (s, 3H), 1.04 (s, 3H). |
| 656 | *** | ** | 561.5 | 1H NMR (400 MHz, ACN-d3) δ 9.07 (s, 1H), 8.69 (s, |
| | | | | 1H), 8.40 (s, 1H), 7.85 (s, 1H), 7.47 (t, J = 59.5 Hz, 1H), |
| | | | | 7.20-7.12 (m, 1H), 7.04-6.96 (m, 1H), 6.94-6.87 (m, |
| | | | | 1H), 5.45 (s, 1H), 4.54-4.51 (m, 2H), 4.20 (s, 1H), 3.74- |
| | | | | 3.65 (m, 2H), 3.34-3.25 (m, 2H), 2.11-2.04 (m, 2H), |
| | | | | 1.91-1.83 (m, 2H), 1.61 (s, 3H), 1.18 (s, 3H), 1.04 (s, 3H). |
| 657 | *** | ** | 560.4 | 1H NMR (400 MHz, DMSO-d6) δ 9.10 (s, 1H), 8.90 (s, |
| | | | | 1H), 8.43 (s, 1H), 7.94 (overlapped t, J = 58.7 Hz, 1H), |
| | | | | 7.89 (overlapped d, J = 0.6 Hz, 1H), 7.52-7.42 (m, 1H), |
| | | | | 7.22 (td, J = 10.0, 2.5 Hz, 1H), 7.08 (td, J = 8.4, 2.3 Hz, |
| | | | | 1H), 5.38 (s, 1H), 5.00 (bs, 1H), 3.57 (s, 2H), 3.43-3.34 |
| | | | | (overlapped m, 4H), 2.62-2.52 (overlapped m, 4H), 1.54 |
| | | | | (s, 3H), 1.11 (s, 3H), 1.00 (s, 3H). |
| 658 | **** | *** | 485.4 | 1H NMR (400 MHz, DMSO-d6) δ 8.28 (s, 1H), 8.06- |
| | | | | 8.02 (m, 1H), 7.61-7.51 (m, 1H), 7.38-7.28 (m, 1H), |
| | | | | 7.23-7.12 (m, 1H), 5.59 (dd, J = 46.2, 8.4 Hz, 1H), 4.68- |
| | | | | 4.52 (m, 2H), 3.97-3.85 (m, 3H), 3.83-3.71 (m, 2H), |
| | | | | 3.52-3.34 (m, 2H, overlapping), 2.92-2.71 (m, 2H), |
| | | | | 2.70-2.55 (m, 2H), 2.15-2.10 (m, 3H), 2.10-1.88 (m, |
| | | | | 2H, overlapping), 1.73-1.56 (m, 1H), 1.54-1.39 (m, |
| | | | | 1H), 1.32-1.25 (m, 1H, overlapping). |
| 659 | **** | *** | 485.5 | 1H NMR (400 MHz, DMSO-d6) δ 8.27 (s, 1H), 8.04 (s, |
| | | | | 1H), 7.62-7.50 (m, 1H), 7.38-7.27 (m, 1H), 7.23- |
| | | | | 7.13 (m, 1H), 5.59 (dd, J = 46.2, 8.4 Hz, 1H), 4.67-4.53 |
| | | | | (m, 2H), 3.98-3.85 (m, 3H), 3.84-3.70 (m, 2H), 3.54- |
| | | | | 3.34 (m, 2H, overlapping), 2.95-2.71 (m, 2H), 2.70- |
| | | | | 2.55 (m, 2H), 2.17-2.09 (m, 3H), 2.09-1.86 (m, 2H, |
| | | | | overlapping), 1.75-1.56 (m, 1H), 1.54-1.39 (m, 1H), |
| | | | | 1.33-1.24 (m, 1H). |
| 660 | ** | ** | 479.4 | 1H NMR (400 MHz, DMSO-d6) δ 8.28 (s, 1H), 8.08- |
| | | | | 7.98 (m, 1H), 7.28-7.14 (m, 2H), 7.06-6.94 (m, 1H), |
| | | | | 4.66-4.53 (m, 2H), 3.89 (s, 3H), 3.83-3.71 (m, 2H), |
| | | | | 3.12 (s, 2H), 2.98-2.70 (m, 4H), 2.17-2.09 (m, 3H), |
| | | | | 2.03-1.92 (m, 1H), 1.79-1.60 (m, 2H), 1.50-1.16 (m, |
| | | | | 2H), 1.12-1.04 (m, 1H), 0.92-0.80 (m, 1H). |
| 661 | *** | ** | 543.4 | 1HNMR (400 MHz, DMSO-d6) δ 9.08 (d, J = 0.7 Hz, |
| | | | | 1H), 9.00 (s, 1H), 8.46 (s, 1H), 8.04 (d, J = 0.7 Hz, 1H), |
| | | | | 7.36-7.27 (m, 2H), 7.05-6.99 (m, 1H), 6.30 (d, J = |
| | | | | 52.3 Hz, 2H), 5.35 (s, 1H), 4.88 (s, 1H), 4.62-4.55 (m, |
| | | | | 1H), 3.60-3.52 (m, 2H), 3.23-3.13 (m, 2H), 2.15- |
| | | | | 2.08 (m, 2H), 1.96-1.86 (m, 2H), 1.56 (s, 3H), 1.10 (s, |
| | | | | 3H), 1.01 (s, 3H). |
| 662 | **** | ** | 560.4 | 1H-NMR (400 MHz, DMSO-d6) δ 9.08 (d, J = 0.7 Hz, |
| | | | | 1H), 9.01 (s, 1H), 8.53 (s, 1H), 8.05 (overlapped d, J = |
| | | | | 0.7 Hz, 1H), 7.97 (overlapped t, J = 58.7 Hz, 1H), 7.52- |
| | | | | 7.44 (m, 1H), 7.22 (ddd, J = 10.3, 9.6, 2.6 Hz, 1H), 7.08 |
| | | | | (td, J = 8.4, 2.1 Hz, 1H), 5.34 (s, 1H), 4.85 (s, 1H), 3.59 |
| | | | | (s, 2H), 3.31-3.24 (m, 4H), 2.64-2.55 (m, 4H), 1.56 (s, |
| | | | | 3H), 1.09 (s, 3H), 1.01 (s, 3H). |
| 663 | **** | ** | 560.3 | 1H-NMR (400 MHz, DMSO-d6) δ 9.04 (d, J = 0.7 Hz, |
| | | | | 1H), 8.97 (s, 1H), 8.49 (s, 1H), 8.01 (overlapped d, J = |
| | | | | 0.7 Hz, 1H), 7.93 (overlapped t, J = 58.6 Hz, 1H), 7.48- |
| | | | | 7.40 (m, 1H), 7.18 (ddd, J = 10.3, 9.6, 2.5 Hz, 1H), 7.04 |
| | | | | (td, J = 8.5, 2.2 Hz, 1H), 5.31 (s, 1H), 4.81 (s, 1H), 3.55 |
| | | | | (s, 2H), 3.27-3.19 (overlapped m, 4H), 2.62-2.52 (m, |
| | | | | 4H), 1.52 (s, 3H), 1.05 (s, 3H), 0.97 (s, 3H). |
| 664 | *** | ** | 551.4 | 1H-NMR (400 MHz, DMSO-d6) δ 9.05 (d, J = 0.7 Hz, |
| | | | | 1H), 8.54 (s, 1H), 8.13 (d, J = 0.6 Hz, 1H), 7.87 (d, J = |
| | | | | 0.7 Hz, 1H), 7.36-7.25 (m, 2H), 7.06-6.98 (m, 1H), |
| | | | | 5.36 (s, 1H), 5.03 (s, 1H), 4.65-4.52 (m, 1H), 3.91 (tt, |
| | | | | J = 7.5, 3.9 Hz, 1H), 3.72-3.56 (m, 2H), 3.31-3.20 |
| | | | | (overlapped m, 2H), 2.14-2.01 (m, 2H), 1.90-1.74 (m, |
| | | | | 2H), 1.54 (s, 3H), 1.18-1.08 (overlapped m, 5H), 1.05- |
| | | | | 0.96 (overlapped m, 5H). |
| 665 | *** | ** | 551.4 | 1H-NMR (400 MHz, DMSO-d6) δ 9.05 (d, J = 0.7 Hz, |
| | | | | 1H), 8.54 (s, 1H), 8.13 (d, J = 0.6 Hz, 1H), 7.87 (d, J = |
| | | | | 0.7 Hz, 1H), 7.36-7.26 (m, 2H), 7.07-6.97 (m, 1H), |
| | | | | 5.36 (s, 1H), 5.03 (s, 1H), 4.64-4.51 (m, 1H), 3.91 (tt, |
| | | | | J = 7.5, 3.9 Hz, 1H), 3.71-3.58 (m, 2H), 3.31-3.21 |
| | | | | (overlapped m, 1H), 2.14-2.02 (m, overlapped), 1.89- |
| | | | | 1.76 (m, 2H), 1.54 (s, 3H), 1.19-1.08 (overlapped m, |
| | | | | 5H), 1.06-0.95 (overlapped m, 5H). |
| 666 | *** | ** | 550.4 | 1H-NMR (400 MHz, DMSO-d6 δ 9.04 (s, 1H), 8.47 (s, |
| | | | | 1H), 8.10 (s, 1H), 7.85 (s, 1H), 7.54-7.43 (m, 1H), 7.22 |
| | | | | (td, J = 10.1, 2.5 Hz, 1H), 7.08 (td, J = 8.3, 2.1 Hz, 1H), |
| | | | | 5.36 (s, 1H), 5.02 (s, 1H), 3.88 (tt, J = 7.5, 3.9 Hz, 1H), |
| | | | | 3.58 (s, 2H), 3.41-3.34 (overlapped m, 4H), 2.61-2.51 |
| | | | | (overlapped m, 4H), 1.53 (s, 3H), 1.17-1.06 (overlapped |
| | | | | m, 5H), 1.06-0.95 (overlapped m, 5H). |
| 667 | *** | ** | 550.5 | 1H-NMR (400 MHz, DMSO-d6 δ 9.04 (s, 1H), 8.47 (s, |
| | | | | 1H), 8.10 (s, 1H), 7.85 (s, 1H), 7.55-7.43 (m, 1H), 7.22 |
| | | | | (td, J = 10.1, 2.5 Hz, 1H), 7.08 (td, J = 8.4, 2.1 Hz, 1H), |
| | | | | 5.36 (s, 1H), 5.02 (s, 1H), 3.88 (tt, J = 7.5, 3.9 Hz, 1H), |
| | | | | 3.58 (s, 2H), 3.41-3.34 (overlapped m, 4H), 2.60-2.53 |
| | | | | (overlapped m, 4H), 1.53 (s, 3H), 1.18-1.07 (overlapped |
| | | | | m, 5H), 1.06-0.93 (overlapped m, 5H). |
| 668 | **** | ** | 528.3 | 1H-NMR (400 MHz, DMSO-d6) δ 9.07 (d, J = 0.7 Hz, |
| | | | | 1H), 9.01 (s, 1H), 8.53 (s, 1H), 8.13-7.79 (overlapping |
| | | | | signals, 2H), 7.45 (td, J = 7.8, 1.8 Hz, 1H), 7.37-7.29 |
| | | | | (m, 1H), 7.23-7.14 (m, 2H), 5.51 (d, J = 5.3 Hz, 1H), |
| | | | | 4.65 (s, 1H), 4.55 (d, J = 5.2 Hz, 1H), 3.62 (s, 2H), 3.31- |
| | | | | 3.23 (m, 4H), 2.64-2.56 (m, 4H), 1.06 (d, J = 2.9 Hz, 6H). |
| 669 | *** | ** | 542.4 | 1H-NMR (400 MHz, DMSO-d6) δ 9.10 (s, 1H), 8.90 (s, |
| | | | | 1H), 8.43 (s, 1H), 8.10-7.77 (overlapping t, 58.8 Hz, |
| | | | | 1H), 7.89 (overlapping s, 1H), 7.47-7.40 (m, 1H), 7.37- |
| | | | | 7.29 (m, 1H), 7.22-7.14 (m, 2H), 5.38 (s, 1H), 5.00 (s, |
| | | | | 1H), 3.60 (s, 2H), 3.41-3.34 (overlapping m, 4H), 2.62- |
| | | | | 2.52 (m, 4H), 1.54 (s, 3H), 1.11 (s, 3H), 0.99 (s, 3H). |
| 670 | *** | *** | 542.3 | 1H-NMR (400 MHz, DMSO-d6) δ 9.10 (s, 1H), 8.91 (s, |
| | | | | 1H), 8.43 (s, 1H), 8.08-7.79 (overlapping t, J = 58.9 Hz, |
| | | | | 1H), 7.89 (overlapping s, 1H), 7.47-7.41 (m, 1H), 7.36- |
| | | | | 7.29 (m, 1H), 7.21-7.14 (m, 2H), 5.38 (s, 1H), 5.00 (s, |
| | | | | 1H), 3.60 (s, 2H), 3.41-3.35 (overlapping m, 4H), 2.62- |
| | | | | 2.51 (m, 4H), 1.54 (s, 3H), 1.11 (s, 3H), 0.99 (s, 3H). |
| 671 | **** | ** | 482.3 | 1H NMR (400 MHz, DMSO-d6): δ 7.45 (dd, J = 11.2, 8.1 |
| | | | | Hz, 1H), 7.25 (ddt, J = 7.1, 5.5, 3.6 Hz, 2H), 7.02-6.95 |
| | | | | (m, 1H), 6.91 (dd, J = 8.1, 3.0 Hz, 1H), 6.37 (s, 2H), 4.51- |
| | | | | 4.42 (m, 1H), 4.39 (s, 2H), 3.59 (t, J = 5.9 Hz, 2H), 3.48- |
| | | | | 3.39 (m, 2H), 3.05-2.96 (m, 2H), 2.93 (t, J = 5.8 Hz, |
| | | | | 2H), 1.89 (d, J = 10.1 Hz, 2H), 1.61 (dt, J = 13.0, 5.9 Hz, 2H). |
| 672 | *** | ** | 525.4 | 1H NMR (400 MHz, ACN-d3δ 9.01 (s, 1H), 8.26 (s, 1H), |
| | | | | 8.14 (s, 1H), 7.81 (s, 1H), 7.19-7.13 (m, 1H), 7.04- |
| | | | | 6.97 (m, 1H), 6.93-6.87 (m, 1H), 4.53-4.50 (m, 2H), |
| | | | | 4.23 (s, 1H), 3.95 (s, 3H), 3.75-3.66 (m, 2H), 3.31-3.27 |
| | | | | (m, 2H), 2.11-2.03 (m, 2H), 1.91-1.84 (m, 2H), 1.60 |
| | | | | (s, 3H), 1.17 (s, 3H), 1.03 (s, 3H). |
| 673 | *** | ** | 525.5 | 1H NMR (400 MHz, ACN-d3) δ 9.01 (s, 1H), 8.26 (s, |
| | | | | 1H), 8.14 (s, 1H), 7.81 (s, 1H), 7.19-7.13 (m, 1H), 7.03- |
| | | | | 6.98 (m, 1H), 6.93-6.87 (m, 1H), 4.55-4.47 (m, 2H), |
| | | | | 4.23 (s, 1H), 3.95 (s, 3H), 3.76-3.67 (m, 2H), 3.33- |
| | | | | 3.24 (m, 2H), 2.11-2.06 (m, 2H), 1.91-1.84 (m, 2H), |
| | | | | 1.60 (s, 3H), 1.17 (s, 3H), 1.03 (s, 3H). |
| 674 | **** | ** | 561.5 | 1HNMR (400 MHz, DMSO-d6) δ 9.10 (d, J = 0.7 Hz, |
| | | | | 1H), 9.08 (s, 1H), 8.57 (s, 1H), 8.06 (d, J = 0.7 Hz, 1H, |
| | | | | overlapped), 7.99 (t, J = 58.6 Hz, 1H, overlapped), 7.37- |
| | | | | 7.27 (m, 2H), 7.05-6.99 (m, 1H), 5.35 (s, 1H), 4.86 (s, |
| | | | | 1H), 4.62-4.54 (m, 1H), 3.59-3.52 (m, 2H), 3.23- |
| | | | | 3.13 (m, 2H), 2.14-2.06 (m, 2H), 1.93-1.83 (m, 2H), |
| | | | | 1.56 (s, 3H), 1.10 (s, 3H), 1.01 (s, 3H). |
| 675 | *** | ** | 524.5 | 1H NMR (400 MHz, DMSO-d6) δ 9.08 (d, J = 0.6 Hz, |
| | | | | 1H), 8.82 (s, 1H), 8.33 (s, 1H), 7.88 (d, J = 0.6 Hz, 1H), |
| | | | | 7.46-7.41 (m, 1H), 7.36-7.29 (m, 1H), 7.21-7.15 (m, |
| | | | | 2H), 6.27 (d, J = 52.4 Hz, 2H), 5.37 (s, 1H), 5.01 (s, 1H), |
| | | | | 3.60 (s, 2H), 3.39-3.36 (m, 4H), 2.61-2.57 (m, 4H), |
| | | | | 1.54 (s, 3H), 1.11 (s, 3H), 0.99 (s, 3H) |
| 676 | *** | ** | 524.5 | 1H NMR (400 MHz, DMSO-d6) δ 9.08 (d, J = 0.6 Hz, |
| | | | | 1H), 8.82 (s, 1H), 8.33 (s, 1H), 7.88 (d, J = 0.6 Hz, 1H), |
| | | | | 7.47-7.41 (m, 1H), 7.36-7.30 (m, 1H), 7.22-7.15 (m, |
| | | | | 2H), 6.27 (d, J = 52.4 Hz, 2H), 5.37 (s, 1H), 5.01 (s, 1H), |
| | | | | 3.60 (s, 2H), 3.41-3.34 (m, 4H), 2.62-2.56 (m, 4H), |
| | | | | 1.54 (s, 3H), 1.11 (s, 3H), 0.99 (s, 3H) |
| 677 | *** | ** | 543.5 | 1H NMR (400 MHz, DMSO-d6) δ 9.09 (d, J = 0.6 Hz, |
| | | | | 1H), 8.88 (s, 1H), 8.36 (s, 1H), 7.89 (d, J = 0.6 Hz, 1H), |
| | | | | 7.36-7.26 (m, 2H), 7.06-6.98 (m, 1H), 6.28 (d, J = |
| | | | | 52.4 Hz, 2H), 5.38 (s, 1H), 5.02 (s, 1H), 4.61-4.53 (m, |
| | | | | 1H), 3.68-3.59 (m, 2H), 3.30-3.21 (m, 2H), 2.12- |
| | | | | 2.05 (m, 2H, overlapped), 1.91-1.81 (m, 2H), 1.54 (s, |
| | | | | 3H), 1.12 (s, 3H), 1.00 (s, 3H). |
| 678 | *** | ** | 543.5 | 1H NMR (400 MHz, DMSO-d6) δ 9.09 (d, J = 0.6 Hz, |
| | | | | 1H), 8.88 (s, 1H), 8.36 (s, 1H), 7.89 (d, J = 0.6 Hz, 1H), |
| | | | | 7.35-7.26 (m, 2H), 7.05-6.99 (m, 1H), 6.28 (d, J = |
| | | | | 52.4 Hz, 2H), 5.38 (s, 1H), 5.02 (s, 1H), 4.62-4.54 (m, |
| | | | | 1H), 3.69-3.60 (m, 2H), 3.30-3.22 (m, 2H), 2.13- |
| | | | | 2.04 (m, 2H, overlapped), 1.91-1.80 (m, 2H), 1.55 (s, |
| | | | | 3H), 1.12 (s, 3H), 1.00 (s, 3H). |
| 679 | ** | ** | 546.4 | 1H NMR (400 MHz, DMSO-d6) δ 9.06 (s, 1H), 8.92 (s, |
| | | | | 1H), 8.71 (s, 1H), 8.45 (s, 1H), 7.95 (t, J = 58.7 Hz, 1H), |
| | | | | 7.52-7.43 (m, 1H), 7.26-7.17 (m, 1H), 7.08 (td, J = |
| | | | | 8.5, 2.3 Hz, 1H), 5.48-5.35 (m, 2H), 4.37 (s, 1H), 3.58 |
| | | | | (s, 2H), 3.43-3.34 (overlapped m, 4H), 2.61-2.54 |
| | | | | (overlapped m, 4H), 1.13 (s, 3H), 0.96 (s, 3H). |
| 680 | **** | *** | 497.4 | 1H NMR (400 MHz, DMSO-d6) δ 9.04 (d, J = 0.6 Hz, |
| | | | | 1H), 8.60 (s, 1H), 8.29-8.23 (m, 1H), 7.80 (s, 1H), 7.38- |
| | | | | 7.25 (m, 2H), 7.07-6.98 (m, 1H), 5.67 (d, J = 5.3 Hz, |
| | | | | 1H), 4.90 (dd, J = 9.2, 6.1 Hz, 1H), 4.61-4.53 (m, 1H), |
| | | | | 3.97 (s, 3H), 3.72 (dd, J = 10.1, 3.6 Hz, 1H), 3.61-3.49 |
| | | | | (overlapping m, 3H), 3.27 (s, 3H), 3.24-3.11 (m, 2H), |
| | | | | 2.17-2.06 (m, 2H), 1.97-1.77 (m, 2H). |
| 681 | **** | *** | 497.4 | 1H NMR (400 MHz, DMSO-d6) δ 9.04 (s, 1H), 8.60 (s, |
| | | | | 1H), 8.26 (s, 1H), 7.80 (s, 1H), 7.38-7.23 (m, 2H), 7.07- |
| | | | | 6.96 (m, 1H), 5.67 (d, J = 5.2 Hz, 1H), 4.96-4.84 (m, |
| | | | | 1H), 4.63-4.52 (m, 1H), 3.97 (s, 3H), 3.72 (dd, J = 10.1, |
| | | | | 3.6 Hz, 1H), 3.61-3.48 (overlapping m, 3H), 3.27 (s, |
| | | | | 3H), 3.24-3.11 (m, 2H), 2.21-2.03 (m, 2H), 1.96- |
| | | | | 1.80 (m, 2H); |
| 682 | **** | ** | 550.42 | 1H NMR (400 MHz, DMSO-d6) δ 9.02 (s, 1H), 8.58 (s, |
| | | | | 1H), 8.22 (s, 1H), 7.99 (s, 1H), 7.53-7.44 (m, 1H), 7.27- |
| | | | | 7.19 (m, 1H), 7.14-7.04 (m, 1H), 5.33 (s, 1H), 4.88 (s, |
| | | | | 1H), 3.90 (tt, J = 7.5, 3.9 Hz, 1H), 3.60 (s, 2H), 3.30- |
| | | | | 3.23 (m, 4H), 2.64-2.56 (m, 4H), 1.55 (s, 3H), 1.17- |
| | | | | 1.11 (m, 2H), 1.09 (s, 3H), 1.07-1.00 (m, 2H), 0.99 (s, 3H). |
| 683 | *** | ** | 550.46 | 1H NMR (400 MHz, DMSO-d6) δ 9.02 (s, 1H), 8.58 (s, |
| | | | | 1H), 8.22 (s, 1H), 7.99 (s, 1H), 7.53-7.44 (m, 1H), 7.28- |
| | | | | 7.19 (m, 1H), 7.12-7.04 (m, 1H), 5.33 (s, 1H), 4.88 (s, |
| | | | | 1H), 3.90 (tt, J = 7.5, 3.9 Hz, 1H), 3.60 (s, 2H), 3.30- |
| | | | | 3.22 (m, 4H), 2.63-2.56 (m, 4H), 1.55 (s, 3H), 1.16- |
| | | | | 1.11 (m, 2H), 1.09 (s, 3H), 1.07-1.00 (m, 2H), 0.99 (s, 3H). |
| 684 | *** | ** | 542.4 | 1H-NMR (400 MHz, DMSO-d6) δ 9.09 (s, 1H), 9.01 (s, |
| | | | | 1H), 8.53 (s, 1H), 8.13-7.81 (overlapping t, J = 7.90 Hz, |
| | | | | 1H), 8.05 (overlapping s, 1H) 7.45 (td, J = 7.8, 1.8 Hz, |
| | | | | 1H), 7.36-7.30 (m, 1H), 7.20-7.15 (m, 2H), 5.35 (bs, |
| | | | | 1H), 4.88 (bs, 1H), 3.62 (s, 2H), 3.31-3.26 (m, 4H), 2.63- |
| | | | | 2.58 (m, 4H), 1.56 (s, 3H), 1.09 (s, 3H), 1.01 (s, 3H). |
| 685 | *** | ** | 528.3 | 1H-NMR (400 MHz, DMSO-d6) δ 9.03 (s, 1H), 8.97 (s, |
| | | | | 1H), 8.49 (s, 1H), 8.10-7.75 (overlapping t, J = 58.6 Hz, |
| | | | | 1H), 7.83 (overlapping s, 1H), 7.41 (td, J = 7.8, 1.8 Hz, |
| | | | | 1H), 7.33-7.26 (m, 1H), 7.19-7.11 (m, 2H), 5.47 (d, |
| | | | | J = 5.3 Hz, 1H), 4.61 (s, 1H), 4.51 (d, J = 5.2 Hz, 1H), 3.58 |
| | | | | (s, 2H), 3.27-3.19 (m, 4H), 2.60-2.54 (m, 4H), 1.01 (d, |
| | | | | J = 3.6 Hz, 6H). |
| 686 | **** | *** | 528.4 | 1H-NMR (400 MHz, DMSO-d6) δ 9.07 (s, 1H), 9.01 (s, |
| | | | | 1H), 8.53 (s, 1H), 8.14-7.81 (overlapping t, J = 58.5 Hz, |
| | | | | 1H), 7.87 (overlapping s, 1H), 7.48-7.42 (m, 1H), 7.37- |
| | | | | 7.29 (m, 1H), 7.22-7.15 (m, 2H), 5.51 (d, J = 5.3 Hz, |
| | | | | 1H), 4.65 (s, 1H), 4.55 (d, J = 5.2 Hz, 1H), 3.62 (s, 2H), |
| | | | | 3.30-3.21 (m, 4H), 2.64-2.57 (m, 4H), 1.06 (d, J = 2.8 |
| | | | | Hz, 6H). |
| 687 | *** | ** | 532.36 | 1H NMR (400 MHz, DMSO-d6) δ 9.10 (s, 1H), 8.90 (s, |
| | | | | 1H), 8.43 (s, 1H), 7.94 (t, J = 58.7 Hz, 1H), 7.70 (s, 1H), |
| | | | | 7.47 (dd, J = 15.5, 8.5 Hz, 1H), 7.25-7.15 (m, 1H), 7.15- |
| | | | | 7.00 (m, 1H), 5.81-5.57 (m, 1H), 4.95-4.83 (m, 1H), |
| | | | | 3.72 (dd, J = 10.1, 3.5 Hz, 1H), 3.63-3.49 (m, 4H), 3.29- |
| | | | | 3.25 (overlapping multiplet, 1H), 3.27 (s, 3H), 2.60- |
| | | | | 2.52 (m, 5H). |
| 688 | *** | *** | 511.4 | 1H NMR (400 MHz, DMSO-d6) δ 9.04 (s, 1H), 8.61 (s, |
| | | | | 1H), 8.27 (s, 1H), 7.94 (s, 1H), 7.38-7.26 (m, 2H), 7.02 |
| | | | | (t, J = 8.6 Hz, 1H), 5.39 (s, 1H), 4.63-4.53 (m, 1H), 3.98 |
| | | | | (s, 3H), 3.66 (d, J = 9.3 Hz, 1H), 3.56 (overlapping d, J = |
| | | | | 8.7 Hz, 1H), 3.54-3.51 (overlapping m, 2H), 3.20 |
| | | | | (overlapping d, J = 1.3 Hz, 3H), 3.19-3.11 (overlapping |
| | | | | m, 2H), 2.19-2.05 (m, 2H), 1.96-1.80 (m, 2H), 1.46 (s, 3H). |
| 689 | *** | *** | 511.4 | 1H NMR (400 MHz, DMSO-d6) δ 9.04 (d, J = 0.6 Hz, |
| | | | | 1H), 8.61 (s, 1H), 8.26 (s, 1H), 7.94 (d, J = 0.6 Hz, 1H), |
| | | | | 7.40-7.24 (m, 2H), 7.08-6.96 (m, 1H), 5.38 (s, 1H), |
| | | | | 4.62-4.52 (m, 1H), 3.97 (s, 3H), 3.66 (d, J = 9.4 Hz, |
| | | | | 1H), 3.56 (overlapping d, J = 9.4 Hz, 1H), 3.54-3.49 |
| | | | | (overlapping m, 2H), 3.20 (overlapping s, 3H), 3.19- |
| | | | | 3.11 (overlapping m, 2H), 2.17-2.04 (m, 2H), 1.94- |
| | | | | 1.80 (m, 2H), 1.46 (s, 3H). |
| 690 | *** | ** | 538.5 | 1H NMR (400 MHz, DMSO-d6) δ 9.05 (d, J = 0.6 Hz, |
| | | | | 1H), 8.48 (s, 1H), 8.13 (s, 1H), 7.86 (d, J = 0.5 Hz, 1H), |
| | | | | 7.52-7.44 (m, 1H), 7.22 (td, J = 10.0, 2.5 Hz, 1H), 7.08 |
| | | | | (td, J = 8.5, 2.4 Hz, 1H), 5.37 (s, 1H), 5.03 (s, 1H), 4.25 |
| | | | | (q, J = 7.2 Hz, 2H), 3.59 (s, 2H), 3.40-3.35 (m, 4H), |
| | | | | 2.58 (s, 4H), 1.54 (s, 3H), 1.43 (t, J = 7.3 Hz, 3H), 1.12 (s, |
| | | | | 3H), 0.99 (s, 3H). |
| 691 | *** | ** | 497.4 | 1H NMR (400 MHz, DMSO-d6) δ 9.04 (s, 1H), 8.61 (s, |
| | | | | 1H), 8.27 (s, 1H), 7.94 (s, 1H), 7.40-7.24 (m, 2H), 7.14- |
| | | | | 6.92 (m, 1H), 5.23 (br. s, 1H), 4.75-4.36 (m, 2H), 3.97 |
| | | | | (s, 3H), 3.73-3.48 (overlapping m, 4H), 3.22-3.04 (m, |
| | | | | 2H), 2.21-2.04 (m, 2H), 1.99-1.80 (m, 2H), 1.45 (s, 3H). |
| 692 | *** | ** | 497.4 | 1H NMR (400 MHz, DMSO-d6) δ 9.01 (s, 1H), 8.57 (s, |
| | | | | 1H), 8.23 (s, 1H), 7.90 (s, 1H), 7.34-7.20 (m, 2H), 6.98 |
| | | | | (d, J = 4.6 Hz, 1H), 5.19 (br. s, 1H), 4.68-4.46 (m, 2H), |
| | | | | 3.93 (s, 3H), 3.65-3.45 (overlapping m, 4H), 3.19-3.07 |
| | | | | (m, 2H), 2.14-1.99 (m, 2H), 1.92-1.78 (m, 2H), 1.41 |
| | | | | (s, 3H). |
| 693 | ** | * | 544.4 | 1H NMR (400 MHz, DMSO-d6) δ 9.17 (s, 1H), 8.92 (s, |
| | | | | 1H), 8.52 (s, 1H), 8.43 (s, 1H), 7.94 (t, J = 58.7 Hz, 1H), |
| | | | | 7.51-7.38 (m, 1H), 7.21 (td, J = 10.1, 2.5 Hz, 1H), 7.07 |
| | | | | (td, J = 8.7, 2.6 Hz, 1H), 5.36 (s, 1H), 3.55 (s, 2H), 3.42- |
| | | | | 3.34 (overlapped m, 4H), 2.56-2.50 (overlapped m, 4H), |
| | | | | 1.42 (s, 6H). |
| 694 | *** | ** | 560.4 | 1H-NMR (400 MHz, DMSO-d6) δ 9.10 (d, J = 0.6 Hz, |
| | | | | 1H), 8.90 (s, 1H), 8.43 (s, 1H), 7.94 (overlapped t, J = |
| | | | | 58.7 Hz, 1H), 7.89 (overlapped d, J = 0.6 Hz, 1H), 7.52- |
| | | | | 7.42 (m, 1H), 7.21 (td, J = 10.0, 2.5 Hz, 1H), 7.07 (td, J = |
| | | | | 8.5, 2.3 Hz, 1H), 5.38 (s, 1H), 5.00 (s, 1H), 3.57 (s, 2H), |
| | | | | 3.42-3.28 (overlapped m, 4H), 2.60-2.53 (overlapped |
| | | | | m, 4H), 1.54 (s, 3H), 1.11 (s, 3H), 1.00 (s, 3H). |
| 695 | *** | ** | 560.4 | 1H-NMR (400 MHz, DMSO-d6) δ 9.10 (d, J = 0.6 Hz, |
| | | | | 1H), 8.90 (s, 1H), 8.43 (s, 1H), 7.94 (overlapped t, J = |
| | | | | 58.7 Hz, 1H), 7.89 (overlapped d, J = 0.6 Hz, 1H), 7.52- |
| | | | | 7.42 (m, 1H), 7.21 (td, J = 10.0, 2.5 Hz, 1H), 7.07 (td, J = |
| | | | | 8.5, 2.3 Hz, 1H), 5.38 (s, 1H), 5.00 (s, 1H), 3.57 (s, 2H), |
| | | | | 3.42-3.28 (overlapped m, 4H), 2.60-2.53 (overlapped |
| | | | | m, 4H), 1.54 (s, 3H), 1.11 (s, 3H), 1.00 (s, 3H). |
| 696 | *** | ** | 511.4 | 1H NMR (400 MHz, DMSO-d6) δ 8.81 (s, 1H), 8.44 (s, |
| | | | | 1H), 8.10 (s, 1H), 7.35-7.27 (m, 2H), 7.06-6.98 (m, |
| | | | | 1H), 6.90 (s, 1H), 4.66 (s, 1H), 4.58 (s, 1H), 4.11 (s, 2H), |
| | | | | 3.95 (s, 3H), 3.68-3.60 (m, 2H), 3.20-3.28 (m, 2H), |
| | | | | 2.11-2.03 (m, 2H), 1.88-1.78 (m, 2H), 1.22 (s, 6H). |
| 697 | *** | ** | 538.5 | 1H NMR (400 MHz, ACN-d3) δ 9.00 (s, 1H), 8.26 (s, |
| | | | | 1H), 8.13 (s, 1H), 7.79 (s, 1H), 7.48-7.40 (m, 1H), 6.99- |
| | | | | 6.91 (m, 2H), 4.51 (s, 1H), 4.23 (q, J = 7.3 Hz, 2H), |
| | | | | 3.60 (s, 2H), 3.43-3.37 (m, 4H), 2.62-2.58 (m, 4H), |
| | | | | 1.60 (s, 3H), 1.48 (t, J = 7.3 Hz, 3H), 1.17 (s, 3H), 1.02 (s, 3H). |
| 698 | *** | ** | 503.5 | 1H NMR (400 MHz, DMSO-d6) δ 8.21-8.17 (m, 1H), |
| | | | | 7.94-7.92 (m, 1H), 7.70-7.61 (m, 1H), 7.52-7.43 (m, |
| | | | | 1H), 7.29-7.20 (m, 1H), 6.1-6.06 (m, 1H), 4.63-4.59 (m, |
| | | | | 2H), 3.92-3.86 (m, 3H), 3.83-3.74 (m, 2H), 3.75-3.60 |
| | | | | (m, 2H), 3.31-3.22 (m, 2H), 2.88 (t, J = 5.9 Hz, 1H), |
| | | | | 2.75 (t, J = 5.8 Hz, 1H), 2.31-2.21 (m, 2H), 2.12 (s, |
| | | | | 1.6H), 2.11 (s, 1.3H). Rotamers present. Two protons |
| | | | | submerged under the DMSO water peak (at 3.3 ppm) |
| 699 | *** | ** | 483.4 | 1H NMR (400 MHz, DMSO-d6) δ 8.26 (s, 1H), 8.02 (s, |
| | | | | 1H), 7.44-7.33 (m, 1H), 7.21-7.11 (m, 1H), 7.08- |
| | | | | 6.97 (m, 1H), 4.62 (s, 0.91H), 4.58 (s, 1.1H), 4.40 (s, 1H), |
| | | | | 3.90 (s, 3H), 3.84-3.69 (m, 2H), 3.19-3.07 (m, 2H), |
| | | | | 3.00 (t, 2H), 2.87 (t, 1H), 2.78-2.71 (m, 3H, merged), |
| | | | | 2.12 (s, 1.65H), 2.10 (s, 1.34H), 1.80-1.64 (m, 2H), 1.53- |
| | | | | 1.42 (m, 2H). Rotamers are present |
| 700 | *** | ** | 531.4 | 1H NMR (400 MHz, DMSO-d6) δ 9.10 (d, J = 0.5 Hz, |
| | | | | 1H), 8.96 (s, 1H), 8.46 (s, 1H), 7.96 (t, J = 58.7 Hz, 1H), |
| | | | | 7.57 (s, 1H), 7.35-7.26 (m, 2H), 7.05-6.98 (m, 1H), |
| | | | | 4.76 (s, 1H), 4.61-4.54 (m, 1H), 3.66-3.57 (m, 2H), |
| | | | | 3.28-3.20 (m, 2H), 2.99 (s, 2H), 2.10-2.02 (m, 2H), |
| | | | | 1.88-1.78 (m, 2H), 1.13 (s, 6H). |
| 701 | ** | ** | 617.4 | 1H NMR (400 MHz, DMSO-d6): δ 7.47-7.40 (m, 1H), |
| | | | | 7.25 (dd, J = 8.6, 3.0 Hz, 3H), 7.22-7.15 (m, 1H), 7.07 |
| | | | | (dt, J = 11.7, 5.9 Hz, 2H), 6.90-6.79 (m, 3H), 4.62- |
| | | | | 4.49 (m, 4H), 3.77 (dd, J = 11.1, 5.5 Hz, 2H), 3.69 (d, J = |
| | | | | 10.7 Hz, 3H), 3.52 (s, 2H), 2.86 (t, J = 5.5 Hz, 1H), 2.73 |
| | | | | (t, J = 5.6 Hz, 1H), 2.44 (d, J = 6.4 Hz, 2H), 2.10 (d, J = |
| | | | | 10.7 Hz, 3H), 1.57 (s, 1H), 1.40 (d, J = 11.6 Hz, 2H), 1.05 |
| | | | | (dd, J = 21.2, 11.6 Hz, 2H). |
| 702 | **** | *** | 497.3 | H NMR (400 MHz, DMSO-d6): δ 7.46 (d, J = 11.6 Hz, |
| | | | | 1H), 7.15 (tdd, J = 9.1, 7.4, 3.8 Hz, 2H), 7.11-7.04 (m, |
| | | | | 1H), 6.95 (s, 1H), 6.47 (s, 2H), 4.58 (d, J = 14.9 Hz, 2H), |
| | | | | 3.82-3.74 (m, 2H), 3.56 (d, J = 11.9 Hz, 2H), 2.88 (t, J = |
| | | | | 5.6 Hz, 1H), 2.75 (t, J = 5.6 Hz, 1H), 2.67 (dd, J = 5.8, 4.0 |
| | | | | Hz, 1H), 2.60 (d, J = 12.0 Hz, 2H), 2.11 (d, J = 11.7 Hz, |
| | | | | 3H), 1.67 (s, 1H), 1.48 (d, J = 11.1 Hz, 2H), 1.21 (d, J = |
| | | | | 11.4 Hz, 2H). |
| 703 | ** | ** | 513.3 | 1H NMR (400 MHz, DMSO-d6): δ 7.44 (dt, J = 20.5, |
| | | | | 10.3 Hz, 1H), 7.16-7.03 (m, 2H), 6.87 (dt, J = 8.0, 2.6 |
| | | | | Hz, 1H), 6.31 (s, 1H), 5.34 (d, J = 4.6 Hz, 1H), 4.55 (t, J = |
| | | | | 12.8 Hz, 3H), 3.77 (q, J = 5.8 Hz, 2H), 3.58 (s, 2H), 2.86 |
| | | | | (t, J = 5.5 Hz, 1H), 2.73 (t, J = 5.7 Hz, 1H), 2.60-2.53 |
| | | | | (m, 2H), 2.10 (d, J = 11.6 Hz, 3H), 1.69 (d, J = 12.0 Hz, |
| | | | | 1H), 1.60 (s, 1H), 1.29-1.10 (m, 3H). |
| 704 | *** | ** | 521.4 | 1H NMR (400 MHz, DMSO-d6) δ 9.03 (s, 1H), 8.63 (s, |
| | | | | 1H), 8.23 (s, 1H), 7.65 (s, 1H), 7.38-7.25 (m, 2H), 7.06- |
| | | | | 6.98 (m, 1H), 4.70 (s, 1H), 4.65-4.52 (m, 1H), 3.93 (tt, |
| | | | | J = 7.5, 3.9 Hz, 1H), 3.60-3.49 (m, 2H), 3.22-3.11 (m, |
| | | | | 2H), 2.99 (s, 2H), 2.17-2.04 (m, 2H), 1.93-1.79 (m, |
| | | | | 2H), 1.18-1.13 (m, 2H), 1.14-1.10 (m, 6H), 1.06- |
| | | | | 0.99 (m, 2H). |
| 705 | *** | ** | 521.5 | 1H NMR (400 MHz, DMSO-d6) δ 9.03 (s, 1H), 8.52 (s, |
| | | | | 1H), 8.12 (s, 1H), 7.52 (s, 1H), 7.37-7.25 (m, 2H), 7.07- |
| | | | | 6.97 (m, 1H), 4.77 (s, 1H), 4.63-4.52 (m, 1H), 3.95- |
| | | | | 3.85 (m, 1H), 3.68-3.57 (m, 2H), 3.24 (t, J = 10.4 Hz, |
| | | | | 2H), 2.97 (s, 2H), 2.13-2.01 (m, 2H), 1.88-1.76 (m, |
| | | | | 2H), 1.18-1.08 (overlapping m, 8H), 1.07-0.98 (m, 2H) |
| 706 | *** | ** | 509.4 | 1H NMR (400 MHz, DMSO-d6) δ 9.04 (s, 1H), 8.53 (s, |
| | | | | 1H), 8.15 (s, 1H), 7.53 (s, 1H), 7.36-7.26 (m, 2H), 7.05- |
| | | | | 6.97 (m, 1H), 4.77 (s, 1H), 4.61-4.53 (m, 1H), 4.26 (q, |
| | | | | J = 7.2 Hz, 2H), 3.66-3.58 (m, 2H), 3.28-3.20 (m, 2H), |
| | | | | 2.97 (s, 2H), 2.12-2.03 (m, 2H), 1.89-1.77 (m, 2H), |
| | | | | 1.43 (t, J = 7.3 Hz, 3H), 1.13 (s, 6H) |
| 707 | *** | ** | 510.4 | 1H-NMR (400 MHz, DMSO-d6) δ 9.05 (s, 1H), 8.94 (s, |
| | | | | 1H), 8.43 (s, 1H), 7.85 (s, 1H), 7.49-7.40 (m, 1H), 7.37- |
| | | | | 7.28 (m, 1H), 7.23-7.14 (m, 2H), 6.29 (d, J = 58.7 Hz, |
| | | | | 2H), 5.50 (s, 1H), 4.66 (s, 1H), 4.55 (s, 1H), 3.62 (s, 2H), |
| | | | | 3.29-3.22 (overlapping m, 4H), 2.67-2.58 (m, 4H), |
| | | | | 1.06 (overlapping s, 3H), 1.05 (overlapping s, 3H). |
| 708 | *** | ** | 542.4 | 1H-NMR (400 MHz, DMSO-d6) δ 9.08 (s, 1H), 9.01 (s, |
| | | | | 1H), 8.53 (s, 1H), 8.14-7.80 (overlapping t, J = 58.5 Hz, |
| | | | | 1H), 8.05 (overlapping s, 1H), 7.48-7.41 (m, 1H), 7.37- |
| | | | | 7.29 (m, 1H), 7.22-7.15 (m, 2H), 5.35 (s, 1H), 4.85 (s, |
| | | | | 1H), 3.62 (s, 2H), 3.30-3.23 (overlapping m, 4H), 2.67- |
| | | | | 2.55 (m, 4H), 1.56 (s, 3H), 1.09 (s, 3H), 1.01 (s, 3H). |
| 709 | *** | ** | 542.4 | 1H-NMR (400 MHz, DMSO-d6) δ 9.08 (s, 1H), 9.01 (s, |
| | | | | 1H), 8.53 (s, 1H), 8.17-7.77 (overlapping t, J = 58.7 Hz, |
| | | | | 1H), 8.05 (overlapping s, 1H) 7.48-7.41 (m, 1H), 7.37- |
| | | | | 7.28 (m, 1H), 7.22-7.14 (m, 2H), 5.35 (s, 1H), 4.85 (s, |
| | | | | 1H), 3.62 (s, 2H), 3.29-3.20 (overlapping m, 4H), 2.70- |
| | | | | 2.52 (m, 4H), 1.56 (s, 3H), 1.09 (s, 3H), 1.01 (s, 3H). |
| 710 | *** | ** | 513.4 | 1H NMR (400 MHz, DMSO-d6) δ 9.08 (s, 1H), 8.87 (s, |
| | | | | 1H), 8.36 (s, 1H), 7.56 (s, 1H), 7.36-7.26 (m, 2H), 7.05- |
| | | | | 6.99 (m, 1H), 6.28 (d, J = 52.4 Hz, 2H), 4.76 (s, 1H), |
| | | | | 4.62-4.53 (m, 1H), 3.68-3.56 (m, 2H), 3.27-3.19 (m, |
| | | | | 2H), 2.98 (s, 2H), 2.13-2.02 (m, 2H), 1.90-1.80 (m, |
| | | | | 2H), 1.13 (s, 6H) |
| 711 | *** | ** | 541.7 | 1H NMR (400 MHz, DMSO) δ 9.06 (s, 1H), 8.93 (s, 1H), |
| | | | | 8.43 (s, 1H), 8.03 (s, 1H), 7.53-7.43 (m, 1H), 7.26- |
| | | | | 7.17 (m, 1H), 7.13-7.02 (m, 1H), 6.29 (d, J = 52.3 Hz, |
| | | | | 2H) 5.35 (s, 1H), 4.87 (s, 1H), 3.59 (s, 2H), 3.30-3.24 |
| | | | | (m, 4H), 2.65-2.58 (m, 4H), 1.56 (s, 3H), 1.09 (s, 3H), |
| | | | | 1.00 (s, 3H). |
| 712 | *** | ** | 541.9 | 1H NMR (400 MHz, DMSO) δ 9.06 (s, 1H), 8.93 (s, 1H), |
| | | | | 8.43 (s, 1H), 8.03 (s, 1H), 7.53-7.44 (m, 1H), 7.26- |
| | | | | 7.18 (m, 1H), 7.12-7.04 (m, 1H), 6.29 (d, J = 52.3 Hz, |
| | | | | 2H), 5.35 (s, 1H), 4.87 (s, 1H), 3.59 (s, 2H), 3.30-3.16 |
| | | | | (overlapping m, 4H), 2.64-2.57 (m, 4H), 1.56 (s, 3H), |
| | | | | 1.09 (s, 3H), 1.00 (s, 3H). |
| 713 | *** | ** | 510.4 | 1H-NMR (400 MHz, DMSO-d6) δ 9.05 (s, 1H), 8.93 (s, |
| | | | | 1H), 8.43 (s, 1H), 7.86 (s, 1H), 7.45 (td, J = 7.7, 1.9 Hz, |
| | | | | 1H), 7.37-7.28 (m, 1H), 7.22-7.15 (m, 2H), 6.28 (d, |
| | | | | J = 52.3 Hz, 2H), 5.49 (s, 1H), 4.65 (s, 1H), 4.55 (s, 1H), |
| | | | | 3.63 (s, 2H), 3.30-3.19 (overlapping m, 4H), 2.65-2.58 |
| | | | | (m, 4H), 1.06 (d, J = 6.1 Hz, 6H). |
| 714 | *** | ** | 531.4 | 1H NMR (400 MHz, DMSO-d6) δ 9.09 (d, J = 0.5 Hz, |
| | | | | 1H), 9.06 (s, 1H), 8.55 (s, 1H), 7.99 (t, J = 58.6 Hz, 1H), |
| | | | | 7.72 (s, 1H), 7.38-7.24 (m, 2H), 7.07-6.97 (m, 1H), |
| | | | | 4.68 (s, 1H), 4.62-4.54 (m, 1H), 3.58-3.49 (m, 2H), |
| | | | | 3.23-3.12 (m, 2H), 3.01 (s, 2H), 2.14-2.06 (m, 2H), |
| | | | | 1.93-1.82 (m, 2H), 1.13 (s, 6H) |
| 715 | *** | ** | 523.7 | 1H-NMR (400 MHz, DMSO-d6) δ 9.06 (s, 1H), 8.94 (s, |
| | | | | 1H), 8.43 (s, 1H), 8.03 (s, 1H), 7.47-7.42 (m, 1H), 7.37- |
| | | | | 7.30 (m, 1H), 7.22-7.16 (m, 2H), 6.29 (d, J = 52.3 Hz, |
| | | | | 2H), 5.35 (s, 1H), 4.87 (s, 1H), 3.62 (s, 2H), 3.29-3.24 |
| | | | | (overlapping m, 4H), 2.66-2.58 (m, 4H), 1.55 (s, 3H), |
| | | | | 1.09 (s, 3H), 1.00 (s, 3H). |
| 716 | *** | ** | 523.8 | 1H-NMR (400 MHz, DMSO-d6) δ 9.02 (s, 1H), 8.90 (s, |
| | | | | 1H), 8.39 (s, 1H), 7.99 (s, 1H), 7.44-7.39 (m, 1H), 7.32- |
| | | | | 7.26 (m, 1H), 7.18-7.12 (m, 2H), 6.25 (d, J = 52.3 Hz, |
| | | | | 2H), 5.31 (s, 1H), 4.83 (s, 1H), 3.58 (s, 2H), 3.25-3.16 |
| | | | | (overlapping m, 4H), 2.63-2.54 (m, 4H), 1.51 (s, 3H), |
| | | | | 1.05 (s, 3H), 0.96 (s, 3H). |
| 717 | *** | ** | 513.4 | 1H-NMR (400 MHz, DMSO-d6) δ 9.03 (s, 1H), 8.95 (s, |
| | | | | 1H), 8.40 (s, 1H), 7.66 (s, 1H), 7.34-7.21 (m, 2H), 7.02- |
| | | | | 6.93 (m, 1H), 6.26 (d, J = 52.3 Hz, 2H), 4.65 (s, 1H), |
| | | | | 4.60-4.46 (m, 1H), 3.56-3.42 (m, 2H), 3.20-3.05 (m, |
| | | | | 2H), 2.96 (s, 2H), 2.14-2.00 (m, 2H), 1.95-1.76 (m, |
| | | | | 2H), 1.09 (s, 6H). |
| 718 | ** | ** | 479.5 | 1H NMR (400 MHz, DMSO-d6) δ 8.34 (s, 1H), 8.05 (d, |
| | | | | J = 2.8 Hz, 1H), 7.27-7.19 (m, 2H), 7.07 (td, J = 8.5, 2.4 |
| | | | | Hz, 1H), 4.62 (d, J = 16.2 Hz, 2H), 3.91 (s, 3H), 3.81- |
| | | | | 3.75 (m, 2H), 3.52-3.42 (over lapped m, 2H), 3.14- |
| | | | | 3.08 (m, 2H), 3.02-2.97 (m, 2H), 2.91-2.85 (m, 1H), |
| | | | | 2.78-2.72 (m, 1H), 2.59-2.54 (m, 2H), 2.12 (d, J = 4.6 |
| | | | | Hz, 3H), 1.93 (s, 3H). |
| 719 | **** | ** | 507.4 | 1H-NMR (400 MHz, ACN-d3) δ 9.03 (s, 1H), 8.26 (s, |
| | | | | 1H), 8.16-8.12 (m, 1H), 7.65 (s, 1H), 7.32-7.23 (m, |
| | | | | 1H), 6.99-6.91 (m, 2H), 6.27 (s, 1H), 4.56 (s, 1H), 4.08 |
| | | | | (bs, 1H), 3.94 (s, 3H), 3.55-3.49 (m, 2H), 3.46-3.40 |
| | | | | (m, 2H), 2.59-2.44 (m, 4H), 1.14 (s, 3H), 1.09 (s, 3H). |
| 720 | **** | ** | 507.4 | 1H-NMR (400 MHz, ACN-d3) δ 9.03 (s, 1H), 8.26 (s, |
| | | | | 1H), 8.14 (s, 1H), 7.65 (s, 1H), 7.34-7.23 (m, 1H), 7.00- |
| | | | | 6.91 (m, 2H), 6.27 (s, 1H), 4.57 (d, J = 4.3 Hz, 1H), |
| | | | | 4.17-4.03 (m, 1H), 3.94 (s, 3H), 3.56-3.50 (m, 2H), |
| | | | | 3.47-3.39 (m, 2H), 2.59-2.45 (m, 4H), 2.12-2.09 |
| | | | | (overlapped m, 1H), 1.14 (s, 3H), 1.09 (s, 3H). |
| 721 | *** | ** | 551.4 | 1H-NMR (400 MHz, DMSO-d6) δ 9.03 (d, J = 0.6 Hz, |
| | | | | 1H), 8.64 (s, 1H), 8.24 (s, 1H), 8.01 (d, J = 0.6 Hz, 1H), |
| | | | | 7.37-7.24 (m, 2H), 7.06-6.96 (m, 1H), 5.32 (s, 1H), |
| | | | | 4.87 (s, 1H), 4.64-4.52 (m, 1H), 4.00-3.87 (m, 1H), |
| | | | | 3.62-3.49 (m, 2H), 3.25-3.12 (m, 2H), 2.19-2.03 (m, |
| | | | | 2H), 1.94-1.77 (m, 2H), 1.56 (s, 3H), 1.19-1.13 (m, |
| | | | | 2H), 1.11 (s, 3H), 1.07-1.02 (m, 2H), 1.01-0.98 (m, 2H). |
| 722 | *** | ** | 551.4 | 1H-NMR (400 MHz, DMSO-d6) δ 9.00 (d, J = 0.6 Hz, |
| | | | | 1H), 8.60 (s, 1H), 8.21 (s, 1H), 7.97 (d, J = 0.6 Hz, 1H), |
| | | | | 7.33-7.20 (m, 1H), 7.03-6.92 (m, 1H), 5.28 (s, 1H), |
| | | | | 4.83 (s, 1H), 4.59-4.47 (m, 1H), 3.94-3.84 (m, 1H), |
| | | | | 3.58-3.46 (m, 2H), 3.21-3.09 (m, 2H), 2.12-2.00 (m, |
| | | | | 2H), 1.89-1.75 (m, 2H), 1.52 (s, 3H), 1.15-1.09 (m, |
| | | | | 2H), 1.07 (s, 3H), 1.03-0.98 (m, 2H), 0.96 (s, 3H). |
| 723 | *** | ** | 528.3 | 1H NMR (400 MHz, DMSO-d6): δ 9.05 (s, 1H), 8.93 (s, |
| | | | | 1H), 8.43 (s, 1H), 7.86 (s, 1H), 7.28 (ddd, J = 11.8, 7.4, |
| | | | | 4.0 Hz, 2H), 7.17 (ddd, J = 12.2, 11.1, 6.5 Hz, 1H), 6.35 |
| | | | | (s, 1H), 6.22 (s, 1H), 4.65 (s, 1H), 4.55 (d, J = 5.3 Hz, |
| | | | | 1H), 3.63 (s, 2H), 3.32 (s, 4H), 2.66 (dd, J = 7.6, 5.7 Hz, |
| | | | | 4H), 1.06 (d, J = 5.9 Hz, 6H). |
| 724 | *** | ** | 528.3 | 1H NMR (400 MHz, DMSO-d6): δ 9.05 (s, 1H), 8.93 (s, |
| | | | | 1H), 8.43 (s, 1H), 7.87 (d, J = 5.2 Hz, 1H), 7.33-7.22 |
| | | | | (m, 2H), 7.17 (ddd, J = 12.1, 8.3, 3.6 Hz, 1H), 6.35 (s, |
| | | | | 1H), 6.22 (s, 1H), 4.65 (s, 1H), 4.55 (d, J = 5.3 Hz, 1H), |
| | | | | 3.63 (s, 2H), 3.32 (s, 4H), 2.66 (dd, J = 7.5, 5.6 Hz, 4H), |
| | | | | 1.06 (d, J = 5.9 Hz, |
| 725 | *** | * | 528.3 | 1H NMR (400 MHz, DMSO-d6): δ 9.07 (s, 1H), 8.82 (s, |
| | | | | 1H), 8.33 (s, 1H), 7.72 (s, 1H), 7.32-7.21 (m, 2H), 7.20- |
| | | | | 7.14 (m, 1H), 6.33 (s, 1H), 6.20 (s, 1H), 4.74 (s, 1H), |
| | | | | 4.53 (d, J = 5.4 Hz, 1H), 3.60 (s, 2H), 3.39 (s, 4H), 2.60 |
| | | | | (s, 4H), 1.07 (d, J = 10.1 Hz, 6H). |
| 726 | *** | ** | 528.3 | 1H NMR (400 MHz, DMSO-d6): δ 9.07 (s, 1H), 8.82 (s, |
| | | | | 1H), 8.33 (s, 1H), 7.72 (s, 1H), 7.32-7.21 (m, 2H), 7.21- |
| | | | | 7.13 (m, 1H), 6.33 (s, 1H), 6.20 (s, 1H), 4.74 (s, 1H), |
| | | | | 4.53 (d, J = 5.4 Hz, 1H), 3.60 (s, 2H), 3.39 (s, 4H), 2.58 |
| | | | | (d, J = 18.8 Hz, 4H), 1.07 (d, J = 10.1 Hz, 6H). |
| 727 | *** | *** | 527.3 | 1H NMR (400 MHz, DMSO-d6): δ 9.05 (s, 1H), 8.82 (s, |
| | | | | 1H), 8.33 (s, 1H), 7.70 (s, 1H), 7.34 (dd, J = 15.5, 8.6 Hz, |
| | | | | 1H), 7.18 (td, J = 10.1, 2.5 Hz, 1H), 7.03 (td, J = 8.4, 2.2 |
| | | | | Hz, 1H), 6.34 (s, 1H), 6.21 (s, 1H), 4.75 (s, 1H), 4.52 (d, |
| | | | | J = 5.4 Hz, 1H), 3.81 (d, J = 10.5 Hz, 2H), 2.83 (t, J = |
| | | | | 13.0 Hz, 2H), 2.61 (d, J = 6.9 Hz, 2H), 1.73 (s, 1H), 1.62 |
| | | | | (d, J = 12.5 Hz, 2H), 1.52-1.40 (m, 2H), 1.05 (t, J = 14.5 |
| | | | | Hz, 6H). |
| 728 | **** | *** | 527.3 | 1H NMR (400 MHz, DMSO-d6): δ 9.05 (s, 1H), 8.82 (s, |
| | | | | 1H), 8.33 (s, 1H), 7.70 (s, 1H), 7.34 (dd, J = 15.5, 8.5 Hz, |
| | | | | 1H), 7.18 (t, J = 8.6 Hz, 1H), 7.03 (t, J = 8.3 Hz, 1H), |
| | | | | 6.34 (s, 1H), 6.21 (s, 1H), 4.75 (s, 1H), 4.52 (d, J = 5.3 |
| | | | | Hz, 1H), 3.81 (d, J = 10.4 Hz, 2H), 2.83 (t, J = 12.7 Hz, |
| | | | | 2H), 2.65-2.57 (m, 2H), 1.73 (s, 1H), 1.62 (d, J = 12.0 |
| | | | | Hz, 2H), 1.47 (s, 2H), 1.07 (d, J = 11.5 Hz, 6H). |
| 729 | ** | ** | 482.4 | 1H NMR (400 MHz, DMSO-d6): δ 8.23 (s, 1H), 7.99 (s, |
| | | | | 1H), 7.51 (dd, J = 15.4, 8.5 Hz, 1H), 7.24-7.16 (m, 1H), |
| | | | | 7.09 (td, J = 8.5, 2.3 Hz, 1H), 4.60 (d, J = 15.3 Hz, 2H), |
| | | | | 3.88 (s, 3H), 3.86-3.75 (m, 3H), 3.03 (s, 4H), 2.88 (t, J = |
| | | | | 5.8 Hz, 1H), 2.74 (t, J = 5.8 Hz, 1H), 2.57 (s, 4H), 2.11 |
| | | | | (d, J = 6.4 Hz, 3H). 1.33(d, j = 6.8 Hz, 1H). |
| 730 | ** | ** | 500.3 | 1H NMR (400 MHz, DMSO-d6): δ 8.18 (d, J = 3.2 Hz, |
| | | | | 1H), 7.92 (s, 1H), 7.49-7.41 (m, 5H), 4.62 (dd, J = 18.5, |
| | | | | 11.8 Hz, 3H), 3.85 (s, 3H), 3.81-3.72 (m, 2H), 3.05 (s, |
| | | | | 4H), 2.87 (t, J = 5.7 Hz, 1H), 2.73 (s, 3H), 2.70-2.61 (m, |
| | | | | 3H), 2.53(m, 1H), 2.52 (s, 1H), 2.11 (d, J = 7.6 Hz, 3H). |
| 731 | ** | * | 536.1 | 1H NMR (400 MHz, DMSO-d6): δ 8.21 (d, J = 2.1 Hz, |
| | | | | 1H), 7.95 (s, 1H), 7.59 (dd, J = 15.1, 8.2 Hz, 1H), 7.47- |
| | | | | 7.40 (m, 1H), 7.25-7.19 (m, 1H), 4.84 (dd, J = 18.2, 9.2 |
| | | | | Hz, 1H), 4.60 (d, J = 15.0 Hz, 2H), 3.86 (s, 3H), 3.82- |
| | | | | 3.75 (m, 2H), 3.05 (s, 4H), 2.87 (t, J = 5.7 Hz, 1H), 2.80- |
| | | | | 2.72 (m, 3H), 2.66 (dd, J = 6.3, 4.4 Hz, 2H), 2.11 (d, J = |
| | | | | 7.3 Hz, 3H). |
| 732 | *** | ** | 518.5 | 1H NMR (400 MHz, DMSO-d6) δ 9.02 (s, 1H), 8.46 (s, |
| | | | | 1H), 8.10 (s, 1H), 7.69 (s, 1H), 7.44 (td, J = 7.7, 1.8 Hz, |
| | | | | 1H), 7.36-7.30 (m, 1H), 7.21-7.15 (m, 2H), 5.45 (d, |
| | | | | J = 5.4 Hz, 1H), 4.74 (s, 1H), 4.50 (d, J = 5.4 Hz, 1H), 3.92- |
| | | | | 3.84 (m, 1H), 3.61 (s, 2H), 3.36 (s, 4H), 2.57 (s, 4H), |
| | | | | 1.15-1.09 (m, 2H), 1.07 (d, J = 4.2 Hz, 3H), 1.04 (s, |
| | | | | 3H), 1.02 (dd, J = 10.2, 5.1 Hz, 2H). |
| 733 | *** | ** | 521.4 | 1H NMR (400 MHz, DMSO-d6) δ 8.98 (s, 1H), 8.47 (s, |
| | | | | 1H), 8.13 (s, 1H), 7.68 (s, 1H), 7.39-7.16 (m, 2H), 7.01 |
| | | | | (t, J = 8.7 Hz, 1H), 4.57 (brs, 1H), 3.95 (s, 3H), 3.65 (brs, |
| | | | | 3H plus 2H N-alpha CH2 of the piperidine), 3.24 (appd, |
| | | | | J = 10.2 Hz, 2H, overlapping), 2.89-2.76 (m, 1H), 2.24- |
| | | | | 2.14 (m, 1H), 2.09-2.06 (m, 2H), 1.84-1.82 (m, 2H), |
| | | | | 1.67-1.57 (m, 1H), 1.59-1.50 (m, 1H). The |
| | | | | stereocenters were assigned tentatively to a trans |
| | | | | configuration. |
| 734 | ** | ** | 521.4 | 1H NMR (400 MHz, DMSO-d6) δ 9.12 (d, J = 2.8 Hz, |
| | | | | 1H), 8.52 (d, J = 2.4 Hz, 1H), 8.18 (d, J = 2.8 Hz, 1H), |
| | | | | 7.92 (d, J = 2.5 Hz, 1H), 7.38-7.24 (m, 2H), 7.03 (d, J = |
| | | | | 9.4 Hz, 1H), 4.58 (brs, 1H), 3.96 (d, J = 2.4 Hz, 3H), 3.72 |
| | | | | (d, J = 3.0 Hz, 3H), 3.66 (brs, 1H), 3.31 (overlapping with |
| | | | | H2O, 4H), 2.66-2.63 (m, 3H), 2.07 (brs, 2H), 1.85 (d, |
| | | | | J = 8.3 Hz, 2H). |
| 735 | *** | ** | 539.5 | 1H NMR (400 MHz, ACN-d3) δ 9.02 (s, 1H), 8.30 (s, |
| | | | | 1H), 8.16 (s, 1H), 7.82 (s, 1H), 7.17 (td, J = 9.3, 5.5 Hz, |
| | | | | 1H), 7.01 (ddd, J = 11.6, 8.8, 3.0 Hz, 1H), 6.93-6.87 (m, |
| | | | | 1H), 4.56-4.49 (m, 2H), 4.28-4.22 (m, 3H), 3.75- |
| | | | | 3.68 (m, 2H), 3.29 (t, J = 9.8 Hz, 2H), 2.13-2.07 |
| | | | | (overlapping m, 2H), 1.92-1.84 (overlapping m, 2H), |
| | | | | 1.61 (s, 3H), 1.49 (t, J = 7.3 Hz, 3H), 1.18 (s, 3H), 1.04 (s, 3H) |
| 736 | *** | ** | 539.5 | 1H NMR (400 MHz, ACN-d3) δ 9.01 (s, 1H), 8.29 (s, |
| | | | | 1H), 8.15 (s, 1H), 7.81 (s, 1H), 7.16 (td, J = 9.3, 5.5 Hz, |
| | | | | 1H), 7.04-6.97 (m, 1H), 6.93-6.87 (m, 1H), 4.54- |
| | | | | 4.47 (m, 2H), 4.28-4.21 (m, 3H), 3.76-3.67 (m, 2H), |
| | | | | 3.33-3.24 (m, 2H), 2.12-2.06 (m, 2H), 1.91-1.85 (m, |
| | | | | 2H), 1.60 (s, 3H), 1.48 (t, J = 7.3 Hz, 3H), 1.17 (s, 3H), |
| | | | | 1.03 (s, 3H). |
| 737 | ** | ** | 507.3 | 1H NMR (400 MHz, DMSO-d6) δ 8.98 (s, 1H), 8.48 (s, |
| | | | | 1H), 8.13 (s, 1H), 7.65 (s, 1H), 7.35-7.25 (m, 2H), 7.06- |
| | | | | 6.97 (m, 1H), 4.62-4.52 (m, 1H), 3.95 (s, 3H), 3.70- |
| | | | | 3.58 (m, 2H), 3.28-3.19 (overlapping, m, 3H), 2.79- |
| | | | | 2.64 (m, 1H), 2.17-2.01 (m, 3H), 1.90-1.76 (m, 2H), |
| | | | | 1.60-1.51 (m, 1H), 1.50-1.42 (m, 1H). COOH signal |
| | | | | was not able to be observed. The stereocenters were |
| | | | | assigned tentatively to a trans configuration. |
| 738 | ** | ** | 453.3 | 1H NMR (400 MHz, DMSO-d6): δ 7.98 (d, J = 5.6 Hz, |
| | | | | 1H), 7.70 (s, 1H), 7.36 (dd, J = 15.6, 8.5 Hz, 1H), 7.20 |
| | | | | (td, J = 10.2, 2.5 Hz, 1H), 7.04 (td, J = 8.5, 2.4 Hz, 1H), |
| | | | | 4.61-4.54 (m, 2H), 3.90 (s, 3H), 3.86-3.71 (m, 2H), |
| | | | | 3.23 (dt, J = 19.8, 8.1 Hz, 3H), 3.09-3.00 (m, 1H), 2.86 |
| | | | | (dd, J = 11.4, 5.7 Hz, 1H), 2.77-2.66 (m, 3H), 2.43 (dt, |
| | | | | J = 14.4, 7.2 Hz, 1H), 2.13 (d, J = 8.6 Hz, 3H), 1.90 (dt, J = |
| | | | | 11.4, 6.6 Hz, 1H), 1.63-1.49 (m, 1H). |
| 739 | *** | ** | 525.4 | 1H NMR (400 MHz, DMSO-d6) δ 9.04 (s, 1H), 8.62 (s, |
| | | | | 1H), 8.28 (s, 1H), 8.01 (s, 1H), 7.37-7.27 (m, 2H), 7.06- |
| | | | | 6.99 (m, 1H), 5.35 (s, 1H), 4.91 (s, 1H), 4.63-4.56 (m, |
| | | | | 1H), 3.98 (s, 3H), 3.61-3.52 (m, 2H), 3.24-3.13 (m, |
| | | | | 2H), 2.16-2.08 (m, 2H), 1.94-1.84 (m, 2H), 1.56 (s, |
| | | | | 3H), 1.11 (s, 3H), 1.01 (s, 3H). |
| 740 | *** | ** | 539.5 | 1H NMR (400 MHz, DMSO-d6) δ 9.04 (s, 1H), 8.65 (s, |
| | | | | 1H), 8.27 (s, 1H), 8.00 (s, 1H), 7.36-7.26 (m, 2H), 7.06- |
| | | | | 6.98 (m, 1H), 5.34 (s, 1H), 4.90 (s, 1H), 4.62-4.54 (m, |
| | | | | 1H), 4.28 (q, J = 7.2 Hz, 2H), 3.60-3.51 (m, 2H), 3.22- |
| | | | | 3.14 (m, 2H), 2.15-2.07 (m, 2H), 1.94-1.83 (m, 2H), |
| | | | | 1.55 (s, 3H), 1.44 (t, J = 7.3 Hz, 3H), 1.10 (s, 3H), 1.00 (s, 3H). |
| 741 | *** | ** | 539.5 | 1H NMR (400 MHz, DMSO-d6) δ 9.04 (s, 1H), 8.65 (s, |
| | | | | 1H), 8.27 (s, 1H), 8.00 (s, 1H), 7.36-7.26 (m, 2H), 7.06- |
| | | | | 6.99 (m, 1H), 5.34 (s, 1H), 4.90 (s, 1H), 4.61-4.55 (m, |
| | | | | 1H), 4.28 (q, J = 7.2 Hz, 2H), 3.60-3.50 (m, 2H), 3.22- |
| | | | | 3.13 (m, 2H), 2.15-2.08 (m, 2H), 1.94-1.83 (m, 2H), |
| | | | | 1.55 (s, 3H), 1.44 (t, J = 7.3 Hz, 3H), 1.10 (s, 3H), 1.00 (s, 3H). |
| 742 | *** | ** | 518.5 | 1H NMR (400 MHz, DMSO-d6) δ 9.02 (s, 1H), 8.47 (s, |
| | | | | 1H), 8.10 (s, 1H), 7.68 (s, 1H), 7.47-7.41 (m, 1H), 7.33 |
| | | | | (m, 1H), 7.22-7.14 (m, 2H), 5.48 (d, J = 5.4 Hz, 1H), |
| | | | | 4.75 (s, 1H), 4.50 (d, J = 5.4 Hz, 1H), 3.91-3.83 (m, |
| | | | | 1H), 3.60 (s, 2H), 3.38-3.34 (overlappping m, 4H), |
| | | | | 2.60-2.52 (m, 4H), 1.14-1.10 (m, 2H), 1.07 (s, 3H), |
| | | | | 1.04 (overlapping s, 3H), 1.03-0.99 (m, 2H). |
| 743 | *** | ** | 513.4 | 1H NMR (400 MHz, DMSO-d6) δ 9.05 (s, 1H), 8.50 (s, |
| | | | | 1H), 8.16 (s, 1H), 7.68 (s, 1H), 7.35-7.26 (m, 2H), 7.06- |
| | | | | 6.98 (m, 1H), 5.84 (d, J = 5.4 Hz, 1H), 4.74 (dd, J = |
| | | | | 14.7, 5.4 Hz, 1H), 4.62-4.53 (m, 1H), 3.96 (s, 3H), 3.69- |
| | | | | 3.60 (m, 2H), 3.30-3.21 (m, 2H), 2.12-2.05 (m, 2H), |
| | | | | 1.90-1.79 (m, 2H), 1.34 (d, J = 13.1 Hz, 3H), 1.30- |
| | | | | 1.25 (m, 3H). |
| 744 | *** | ** | 513.4 | 1H NMR (400 MHz, DMSO-d6) δ 9.05 (s, 1H), 8.51 (s, |
| | | | | 1H), 8.16 (s, 1H), 7.68 (s, 1H), 7.36-7.27 (m, 2H), 7.06- |
| | | | | 6.99 (m, 1H), 5.85 (d, J = 5.4 Hz, 1H), 4.74 (dd, J = |
| | | | | 14.7, 5.4 Hz, 1H), 4.61-4.55 (m, 1H), 3.96 (s, 3H), 3.69- |
| | | | | 3.61 (m, 2H), 3.30-3.21 (m, 2H), 2.13-2.05 (m, 2H), |
| | | | | 1.91-1.79 (m, 2H), 1.34 (d, J = 13.1 Hz, 3H), 1.29 (d, J = |
| | | | | 12.9 Hz, 3H). |
| 745 | ** | ** | 507.3 | 1H NMR (400 MHz, DMSO-d6) δ 9.12 (s, 1H), 8.53 (s, |
| | | | | 1H), 8.18 (d, J = 0.6 Hz, 1H), 7.98 (s, 1H), 7.86 (d, J = |
| | | | | 15.6 Hz, 1H), 7.36-7.24 (m, 2H), 7.06-6.99 |
| | | | | (overlapping m, 1H), 7.00 (overlapping d, J = 15.6 Hz, |
| | | | | 1H), 4.65-4.53 m, 1H), 3.96 (s, 3H), 3.76 (s, 3H), 3.71- |
| | | | | 3.62 (m, 2H), 3.30-3.23 (overlapping m, 2H), 2.12- |
| | | | | 2.02 (m, 2H), 1.90-1.79 (m, 2H). |
| 746 | *** | ** | 518.4 | 1H-NMR (400 MHz, DMSO-d6) δ 9.01 (d, J = 0.6 Hz, |
| | | | | 1H), 8.58 (s, 1H), 8.25-8.18 (m, 1H), 7.82 (s, 1H), 7.50- |
| | | | | 7.41 (m, 1H), 7.39-7.29 (m, 1H), 7.24-7.13 (m, 2H), |
| | | | | 5.48 (d, J = 5.3 Hz, 1H), 4.67 (d, J = 4.2 Hz, 1H), 4.53 (d, |
| | | | | J = 5.2 Hz, 1H), 3.90 (tq, J = 7.5, 3.6 Hz, 1H), 3.65 (d, J = |
| | | | | 10.6 Hz, 2H), 3.33-3.20 (overlapping m, 4H), 2.65- |
| | | | | 2.53 (m, 4H), 1.18-1.11 (m, 2H), 1.06 (s, 3H), 1.05- |
| | | | | 1.03 (overlapping m, 4H), 1.03-1.01 (m, 1H). |
| 747 | *** | ** | 532.4 | 1H-NMR (400 MHz, DMSO-d6) δ 9.04 (d, J = 0.6 Hz, |
| | | | | 1H), 8.47 (s, 1H), 8.10 (d, J = 2.1 Hz, 1H), 7.85 (d, J = |
| | | | | 0.6 Hz, 1H), 7.44 (td, J = 7.8, 1.8 Hz, 1H), 7.38-7.29 |
| | | | | (m, 1H), 7.22-7.14 (m, 2H), 5.37 (s, 1H), 5.03 (s, 1H), |
| | | | | 3.88 (tt, J = 7.5, 3.9 Hz, 1H), 3.61 (s, 2H), 3.39-3.34 |
| | | | | (overlapping m, 4H), 2.63-2.53 (m, 4H), 1.53 (s, 3H), |
| | | | | 1.15-1.12 (overlapping m, 2H), 1.11 (overlapping s, |
| | | | | 3H), 1.05-1.00 (m, 2H), 0.99 (s, 3H). |
| 748 | NA | NA | 465.40 | 1H NMR (400 MHz, DMSO-d6) δ 9.13 (d, J = 0.6 Hz, |
| | | | | 1H), 8.90 (s, 1H), 8.48 (d, J = 0.6 Hz, 1H), 8.37 (s 1H), |
| | | | | 8.20 (formate s, 0.6H), 7.54-7.41 (m, 1H), 7.21 (td, J = |
| | | | | 10.0, 2.6 Hz, 1H), 7.07 (td, J = 8.4, 2.2 Hz, 1H), 6.28 (d, |
| | | | | J = 52.1 Hz, 2H), 3.57 (s, 2H), 3.48-3.38 (m, 4H), 2.62- |
| | | | | 2.53 (m, 4H). |
| 749 | NA | NA | 455.40 | 1H NMR (400 MHz, DMSO-d6) δ 8.89 (s, 1H), 8.65 (s, |
| | | | | 1H), 8.40 (s, 1H), 7.54-7.43 (m, 1H), 7.22 (td, J = 10.0, |
| | | | | 2.5 Hz, 1H), 7.08 (td, J = 8.5, 2.5 Hz, 1H), 6.65 (s, 1H), |
| | | | | 6.27 (d, J = 52.4 Hz, 2H), 6.18 (s, 2H), 3.60 (s, 2H), 3.15- |
| | | | | 3.05 (m, 4H), 2.64-2.57 (m, 4H). |
| 750 | NA | NA | 560.50 | 1H-NMR (400 MHz, CD3CN) δ 9.04 (d, J = 0.7 Hz, 1H), |
| | | | | 8.79 (s, 1H), 8.48 (s, 1H), 7.99 (d, J = 0.7 Hz, 1H), 7.47 |
| | | | | (t, J = 59.5 Hz, 1H), 7.27-7.19 (m, 1H), 7.11 (td, J = 9.2, |
| | | | | 4.6 Hz,, 1H), 7.07-6.99 (m, 1H), 4.57-4.41 (m, 1H), |
| | | | | 4.11 (bs, 1H), 3.63 (s, 2H), 3.39-3.29 (m, 4H), 2.72- |
| | | | | 2.57 (m, 4H), 1.62 (s, 3H), 1.16 (s, 3H), 1.04 (s, 3H). |
| 751 | NA | NA | 456.40 | 1H-NMR (400 MHz, DMSO-d6) δ 11.05 (bs, 1H), 8.92 |
| | | | | (s, 1H), 8.69 (s, 1H), 8.41 (s, 1H), 7.48 (dd, J = 15.4, 8.6 |
| | | | | Hz, 1H), 7.26-7.16 (m, 1H), 7.08 (td, J = 8.5, 2.2 Hz, |
| | | | | 1H), 6.84 (s, 1H), 6.28 (d, J = 52.3 Hz, 2H), 3.60 (s, 2H), |
| | | | | 3.23-3.10 (m, 4H), 2.65-2.55 (m, 4H). Plausibly |
| | | | | tautomers present |
| 752 | NA | NA | 546.50 | 1H NMR (400 MHz, CD3CN) δ 8.95 (s, 1H), 8.54 (s, |
| | | | | 1H), 8.30 (s, 1H), 7.44 (dt, J = 15.1, 7.5 Hz, 1H), 7.09- |
| | | | | 6.83 (m, 2H), 6.13 (d, J = 52.6 Hz, 2H), 4.85 (d, J = 1.9 |
| | | | | Hz, 1H), 3.59 (s, 2H), 3.51-3.36 (m, 4H), 2.67-2.51 |
| | | | | (m, 4H), 1.22 (s, 3H), 1.13 (s, 3H). OH peaks were not |
| | | | | observed. |
| 753 | NA | NA | 528.50 | 1H-NMR (400 MHz, DMSO-d6) δ 8.88 (s, 1H), 8.79 (s, |
| | | | | 1H), 8.37 (s, 1H), 7.44 (dd, J = 15.5, 8.5 Hz, 1H), 7.18 |
| | | | | (td, J = 10.0, 2.5 Hz, 1H), 7.09-6.99 (m, 2H), 6.25 (d, |
| | | | | J = 52.2 Hz, 2H), 4.63 (s, 1H), 4.07 (s, 2H), 3.56 (s, 2H), |
| | | | | 3.17 (m, 4H), 2.61-2.57 (m, 4H), 1.18 (s, 6H). |
| 754 | NA | NA | 496.30 | 1H NMR (400 MHz, DMSO) δ 9.01 (d, J = 0.4 Hz, 1H), |
| | | | | 8.57 (s, 1H), 8.31 (s, 1H), 7.83 (s, 1H), 7.49 (dd, J = 15.4, |
| | | | | 8.5 Hz, 1H), 7.22 (td, J = 10.2, 2.5 Hz, 1H), 7.08 (td, J = |
| | | | | 8.4, 2.1 Hz, 1H), 5.47 (d, J = 5.3 Hz, 1H), 4.67 (s, 1H), |
| | | | | 4.54 (d, J = 5.0 Hz, 1H), 3.59 (s, 2H), 3.27 (s, 4H), 2.60 |
| | | | | (s, 4H), 1.06 (d, J = 9.2 Hz, 6H). |
| 755 | NA | NA | 496.30 | 1H NMR (400 MHz, DMSO-d6) δ 9.01 (s, 1H), 8.56 (s, |
| | | | | 1H), 8.30 (s, 1H), 7.83 (s, 1H), 7.49 (dd, J = 15.4, 8.5 Hz, |
| | | | | 1H), 7.22 (td, J = 10.2, 2.5 Hz, 1H), 7.08 (td, J = 8.4, 2.2 |
| | | | | Hz, 1H), 5.47 (d, J = 5.2 Hz, 1H), 4.67 (s, 1H), 4.54 (d, |
| | | | | J = 4.9 Hz, 1H), 3.59 (s, 2H), 3.27 (s, 4H), 2.60 (s, 4H), |
| | | | | 1.06 (d, J = 9.2 Hz, 6H). |
| 756 | *** | ** | 560.50 | 1H-NMR (400 MHz, CD3CN) δ 9.04 (s, 1H), 8.79 (s, |
| | | | | 1H), 8.48 (s, 1H), 7.99 (s, 1H), 7.47 (t, J = 59.5 Hz, 1H), |
| | | | | 7.23 (ddd, J = 9.1, 5.6, 3.3 Hz, 1H), 7.15-6.96 (m, 2H), |
| | | | | 4.50 (s, 1H), 4.09 (bs, 1H), 3.63 (s, 2H), 3.39-3.28 (m, |
| | | | | 4H), 2.70-2.61 (m, 4H), 1.62 (s, 3H), 1.16 (s, 3H), 1.04 |
| | | | | (s, 3H). |
| 757 | *** | ** | 527.50 | 1H NMR (400 MHz, DMSO-d6) δ 8.88 (s, 1H), 8.68 (s, |
| | | | | 1H), 8.39 (s, 1H), 7.53-7.44 (m, 1H), 7.22 (td, J = 10.1, |
| | | | | 2.6 Hz, 1H), 7.08 (td, J = 8.6, 2.5 Hz, 1H), 6.75 (s, 1H), |
| | | | | 6.48 (t, J = 6.0 Hz, 1H), 6.28 (d, J = 52.3 Hz, 2H), 4.57 (s, |
| | | | | 1H), 3.60 (s, 2H), 3.27 (overlapped d, J = 6.0 Hz, 2H), |
| | | | | 3.18-3.05 (m, 4H), 2.65-2.55 (m, 4H), 1.15 (s, 6H). |
| 758 | *** | ** | 514.50 | 1H-NMR (400 MHz, DMSO-d6) δ 8.92 (s, 1H), 8.84 (s, |
| | | | | 1H), 8.41 (s, 1H), 7.48 (dd, J = 15.5, 8.5 Hz, 1H), 7.22 |
| | | | | (td, J = 10.1, 2.5 Hz, 1H), 7.11 (s, 1H), 7.08 (td, J = 8.4, |
| | | | | 2.5 Hz, 1H), 6.28 (d, J = 52.3 Hz, 2H), 4.45 (dd, J = 5.4, |
| | | | | 3.9 Hz, 2H), 3.71 (dd, J = 5.4, 3.9 Hz, 2H), 3.60 (s, 2H), |
| | | | | 3.31 (s, 3H), 3.26-3.16 (m, 4H), 2.65-2.55 (m, 4H). |
| 759 | *** | ** | 499.50 | 1H NMR (400 MHz, DMSO-d6) δ 8.89 (s, 1H), 8.70 (d, |
| | | | | J = 0.5 Hz, 1H), 8.39 (s, 1H), 7.53-7.44 (m, 1H), 7.22 (td, |
| | | | | J = 10.0, 2.6 Hz, 1H), 7.08 (td, J = 8.5, 2.1 Hz, 1H), 6.70- |
| | | | | 6.61 (m, 2H), 6.27 (d, J = 52.4 Hz, 2H), 4.75 (t, J = 5.5 |
| | | | | Hz, 1H), 3.60 (overlapped s, 2H), 3.59-3.55 (overlapped |
| | | | | m, 2H), 3.38-3.33 (overlapped m, 2H), 3.19-3.06 (m, |
| | | | | 4H), 2.65-2.56 (m, 4H). |
| 760 | *** | *** | 500.50 | 1H NMR (400 MHz, CD3CN) δ 8.78 (s, 1H), 8.68 (s, |
| | | | | 1H), 8.40 (s, 1H), 7.53-7.36 (m, 1H), 7.09 (s, 1H), 7.03- |
| | | | | 6.87 (m, 2H), 6.14 (d, J = 56 Hz, 2H), 4.47-4.32 (m, |
| | | | | 2H), 3.85 (dd, J = 9.1, 4.7 Hz, 2H), 3.61 (s, 2H), 3.24 (m, |
| | | | | 2H), 3.12 (t, J = 5.4 Hz, 1H), 2.70-2.53 (m, 4H). |
| 761 | *** | ** | 474.40 | 1H NMR (500 MHz, DMSO-d6) δ 8.96 (d, J = 0.7 Hz, |
| | | | | 1H), 8.92 (s, 1H), 8.39 (s, 1H), 7.91 (d, J = 0.7 Hz, 1H), |
| | | | | 7.52-7.43 (m, 1H), 7.22 (ddd, J = 10.2, 9.5, 2.6 Hz, 1H), |
| | | | | 7.08 (td, J = 8.5, 2.3 Hz, 1H), 6.28 (d, J = 52.2 Hz, 2H), |
| | | | | 3.58 (s, 2H), 3.33-3.26 (overlapped m, 4H), 2.61-2.54 |
| | | | | (m, 4H). |
| 762 | ** | ** | 513.50 | 1H NMR (500 MHz, DMSO-d6) δ 8.89 (s, 1H), 8.70 (s, |
| | | | | 1H), 8.40 (s, 1H), 7.52-7.43 (m, 1H), 7.22 (td, J = 9.9, |
| | | | | 2.5 Hz, 1H), 7.08 (td, J = 8.3, 2.3 Hz, 1H), 6.75 (t, J = 5.6 |
| | | | | Hz, 1H), 6.67 (s, 1H), 6.27 (d, J = 52.4 Hz, 2H), 3.57- |
| | | | | 3.53 (m, 1H), 3.53-3.49 (m, 2H), 3.49-3.44 (m, 2H), |
| | | | | 3.28 (s, 3H), 3.15-3.07 (m, 3H), 2.66-2.56 (m, 4H). |
| | | | | Rotamers present (9:1 ratio) |
| 763 | **** | *** | 507.50 | 1H NMR (500 MHz, DMSO-d6) δ 9.01 (d, J = 0.8 Hz, |
| | | | | 1H), 8.63 (s, 1H), 8.28 (d, J = 0.7 Hz, 1H), 7.82 (d, J = |
| | | | | 0.6 Hz, 1H), 7.39-7.30 (m, 1H), 7.30-7.21 (m, 1H), |
| | | | | 7.08 (td, J = 8.5, 2.4 Hz, 1H), 6.27 (s, 1H), 5.49 (d, J = |
| | | | | 5.3 Hz, 1H), 4.69 (s, 1H), 4.54 (d, J = 5.0 Hz, 1H), 3.97 |
| | | | | (s, 3H), 3.41-3.36 (m, 2H), 3.33-3.28 (m, 2H), 2.60- |
| | | | | 2.53 (m, 2H), 2.52-2.50 (overlapping m, 2H), 1.06 (s, |
| | | | | 3H), 1.04 (s, 3H). |
| 764 | **** | *** | 507.50 | 1H NMR (500 MHz, DMSO-d6) δ 9.01 (d, J = 0.7 Hz, |
| | | | | 1H), 8.63 (s, 1H), 8.28 (d, J = 0.7 Hz, 1H), 7.82 (s, 1H), |
| | | | | 7.39-7.31 (m, 1H), 7.28-7.22 (m, 1H), 7.08 (td, J = |
| | | | | 8.5, 2.5 Hz, 1H), 6.27 (s, 1H), 5.49 (d, J = 5.3 Hz, 1H), |
| | | | | 4.69 (s, 1H), 4.54 (d, J = 5.1 Hz, 1H), 3.97 (s, 3H), 3.43- |
| | | | | 3.36 (m, 2H), 3.32-3.28 (m, 2H), 2.62-2.52 (m, 2H), |
| | | | | 2.52-2.50 (overlapping m, 2H), 1.06 (s, 3H), 1.04 (s, 3H). |
| 765 | ** | ** | 484.30 | 1H NMR (400 MHz, DMSO) δ 9.12 (s, 1H), 8.92 (s, 1H), |
| | | | 484.40 | 8.41 (s, 1H), 8.34 (s, 1H), 7.48 (dd, J = 15.4, 8.5 Hz, 1H), |
| | | | | 7.22 (td, J = 10.1, 2.5 Hz, 1H), 7.13-7.03 (m, 1H), 6.28 |
| | | | | (d, J = 52.2 Hz, 2H), 3.57 (s, 4H), 3.38 b(s, 4H), 2.57 (bs, 4H). |
| | | | | 1H NMR (400 MHz, DMSO-d6) δ 9.11 (s, 1H), 8.92 (s, |
| | | | | 1H), 8.41 (s, 1H), 8.32 (s, 1H), 7.48 (dd, J = 15.4, 8.5 Hz, |
| | | | | 1H), 7.21 (td, J = 10.1, 2.5 Hz, 1H), 7.08 (td, J = 8.5, 2.3 |
| | | | | Hz, 1H), 6.28 (d, J = 52.2 Hz, 2H), 6.22 (s, 1H), 3.59 (m, |
| | | | | 2H), 3.36 (m, merging with water peak, 4H), 2.60 (m, |
| | | | | 4H). (Acid, O—H peak is not apparent) |
| 766 | *** | ** | 543.50 | 1H NMR (500 MHz, DMSO-d6) δ 9.08 (s, 1H), 9.07 (d, |
| | | | | J = 0.7 Hz, 1H), 8.57 (s, 1H), 7.97 (merged t, J = 58.7 Hz, |
| | | | | 1H), 7.89 (merged s, 1H), 7.37-7.31 (m, 1H), 7.26- |
| | | | | 7.21 (m, 1H), 7.08 (td, J = 8.4, 2.3 Hz, 1H), 6.27 (s, 1H), |
| | | | | 5.48 (d, J = 5.3 Hz, 1H), 4.64 (s, 1H), 4.56 (d, J = 5.2 Hz, |
| | | | | 1H), 3.43-3.35 (m, 2H), 3.34-3.31 (overlapping m, |
| | | | | 2H), 2.59-2.53 (m, 2H), 2.49-2.45 (overlapping m, |
| | | | | 2H), 1.07 (s, 3H), 1.06 (s, 3H). |
| 767 | *** | ** | 543.50 | 1H NMR (500 MHz, DMSO-d6) δ 9.08 (d, J = 0.4 Hz, |
| | | | | 1H), 9.07 (d, J = 0.8 Hz, 1H), 8.61-8.52 (m, 1H), 7.97 |
| | | | | (merged t, J = 58.6 Hz, 1H), 7.89 (merged s, 1H), 7.37- |
| | | | | 7.30 (m, 1H), 7.27-7.21 (m, 1H), 7.07 (td, J = 8.5, 2.4 |
| | | | | Hz, 1H), 6.27 (s, 1H), 5.48 (d, J = 5.3 Hz, 1H), 4.64 (s, |
| | | | | 1H), 4.56 (d, J = 5.2 Hz, 1H), 3.42-3.36 (m, 2H), 3.34- |
| | | | | 3.31 (overlapping m, 2H), 2.59-2.53 (m, 2H), 2.49- |
| | | | | 2.45 (overlapping m, 2H), 1.07 (s, 3H), 1.06 (s, 3H). |
| 768 | ** | ** | 493.40 | 1H-NMR (400 MHz, DMSO-d6) δ 8.97 (s, 1H), 8.47 (s, |
| | | | | 1H), , 8.13 (s, 1H), 7.48 (s, 1H), 7.35-7.21 (m, 2H), 7.10- |
| | | | | 6.88 (m, 1H), 4.62-4.52 (m, 1H), 3.94 (s, 3H), 3.66- |
| | | | | 3.59 (m, 2H), 3.52 (dd, J = 11.3, 5.7 Hz, 1H, OH), 3.43- |
| | | | | 3.37 (m, overlapping 2H), 3.26-3.19 (m, overlapping |
| | | | | 2H), 2.14 (appdt, J = 8.7, 4.5 Hz, 1H), 2.11-2.03 (m, |
| | | | | 2H), 1.90-1.76 (m, 2H), 1.68-1.59 (m, 1H), 1.17 |
| | | | | (appdt, J = 8.6, 4.1 Hz, 1H), 0.95 (ddd, J = 9.2, 5.5, 3.8 |
| | | | | Hz, 1H). The diastereocarbons are not assigned. |
| 769 | *** | ** | 533.40 | 1H-NMR (400 MHz, DMSO-d6) δ 9.03 (s, 1H), 8.54 |
| | | | | (s, 1H), 8.14 (s, 1H), 7.69 (s, 1H), 7.39-7.30 (m, 1H), |
| | | | | 7.24 (td, J = 8.5, 2.4 Hz, 1H), 7.07 (td, J = 8.5, 2.4 Hz, |
| | | | | 1H), 6.26 (s, 1H), 5.49 (d, J = 5.4 Hz, 1H), 4.77 (s, 1H), |
| | | | | 4.51 (d, J = 5.3 Hz, 1H), 3.90 (tt, J = 7.5, 3.9 Hz, 1H), |
| | | | | 3.54-3.44 (m, 2H), 3.44-3.37 (m, 2H), 2.56-2.51 |
| | | | | (overlapping m, 2H), 2.48-2.42 (m, 2H), 1.18-1.12 (m, |
| | | | | 2H), 1.08 (s, 3H), 1.05 (s, 3H), 1.04-0.98 (m, 2H). |
| 770 | *** | ** | 533.40 | 1H-NMR (400 MHz, DMSO-d6) δ 9.03 (s, 1H), 8.54 (s, |
| | | | | 1H), 8.14 (s, 1H), 7.69 (s, 1H), 7.38-7.32 (m, 1H), 7.24 |
| | | | | (td, J = 8.5, 2.4 Hz, 1H), 7.07 (td, J = 8.5, 2.4 Hz, 1H), |
| | | | | 6.26 (s, 1H), 5.49 (d, J = 5.4 Hz, 1H), 4.77 (s, 1H), 4.51 |
| | | | | (d, J = 5.4 Hz, 1H), 3.90 (tt, J = 7.5, 3.9 Hz, 1H), 3.52- |
| | | | | 3.46 (m, 2H), 3.45-3.36 (m, 2H), 2.56-2.51 |
| | | | | (overlapping m, 2H), 2.47-2.38 (m, 2H), 1.18-1.12 (m, |
| | | | | 2H), 1.08 (s, 3H), 1.05 (s, 3H), 1.04-0.99 (m, 2H). |
| 771 | *** | *** | 525.30 | 1H NMR (400 MHz, DMSO-d6): δ 9.05 (s, 1H), 9.02 (s, |
| | | | | 1H), 8.47 (s, 1H), 7.86 (s, 1H), 7.37 (q, J = 8.7 Hz, 1H), |
| | | | | 7.24 (td, J = 10.1, 2.5 Hz, 1H), 7.08 (tt, J = 6.6, 3.2 Hz, |
| | | | | 1H), 6.36 (s, 1H), 6.27 (s, 1H), 6.23 (s, 1H), 5.53 (d, J = |
| | | | | 5.3 Hz, 1H), 4.66 (s, 1H), 4.56 (d, J = 5.2 Hz, 1H), 3.40- |
| | | | | 3.27 (m, 4H), 2.59 (d, J = 7.0 Hz, 4H), 1.07 (d, J = 5.7 |
| | | | | Hz, 6H). |
| 772 | *** | *** | 524.40 | 1H-NMR (400 MHz, DMSO-d6) δ 9.02 (d, J = 0.6 Hz, |
| | | | | 1H), 8.56 (s, 1H), 8.24 (d, J = 0.5 Hz, 1H), 7.99 (s, 1H), |
| | | | | 7.49 (dd, J = 15.5, 8.6 Hz, 1H), 7.26-7.19 (m, 1H), 7.08 |
| | | | | (td, J = 8.4, 2.3 Hz, 1H), 5.34 (s, 1H), 4.89 (s, 1H), 3.96 |
| | | | | (s, 3H), 3.60 (s, 2H), 3.31-3.20 (overlapped m, 4H), |
| | | | | 2.67-2.56 (m, 4H), 1.55 (s, 3H), 1.10 (s, 3H), 0.99 (s, 3H). |
| 773 | *** | *** | 524.40 | 1H-NMR (400 MHz, DMSO-d6) δ 9.02 (d, J = 0.6 Hz, |
| | | | | 1H), 8.56 (s, 1H), 8.24 (s, 1H), 7.99 (d, J = 0.5 Hz, 1H), |
| | | | | 7.49 (dd, J = 15.4, 8.6 Hz, 1H), 7.27-7.17 (m, 1H), 7.08 |
| | | | | (td, J = 8.6, 2.3 Hz, 1H), 5.34 (s, 1H), 4.89 (s, 1H), 3.96 |
| | | | | (s, 3H), 3.60 (s, 2H), 3.31-3.22 (overlapped m, 4H), |
| | | | | 2.67-2.56 (m, 4H), 1.55 (s, 3H), 1.10 (s, 3H), 0.99 (s, 3H). |
| 774 | ** | ** | 574.30 | 1H NMR (400 MHz, DMSO-d6) δ 9.15 (d, J = 0.6 Hz, |
| | | | | 1H), 8.98 (s, 1H), 8.93 (s, 1H), 8.52 (s, 1H), 8.50 (s, 1H), |
| | | | | 8.28 (s, 1H), 7.99 (t, J = 58.6 Hz, 1H), 7.89 (t, J = 58.6 Hz, |
| | | | | 1H), 7.32-7.21 (m, 2H), 7.21-7.14 (m, 1H), 3.63 (s, |
| | | | | 2H), 3.31 (overlap, 4H), 2.62 (s, 4H). |
| 775 | *** | ** | 532.40 | 1H-NMR (400 MHz, DMSO-d6) δ 9.02 (s, 1H), 8.58 (s, |
| | | | | 1H), 8.22 (s, 1H), 7.99 (s, 1H), 7.45 (td, J = 7.8, 1.7 Hz, |
| | | | | 1H), 7.39-7.30 (m, 1H), 7.24-7.12 (m, 2H), 5.34 (s, |
| | | | | 1H), 4.89 (s, 1H), 3.90 (tt, J = 7.5, 3.9 Hz, 1H), 3.63 (s, |
| | | | | 2H), 3.30-3.19 (m, overalpping 4H), 2.66-2.52 (m, |
| | | | | 4H), 1.55 (s, 3H), 1.15-1.12 (m, 2H), 1.10 (s, 3H), 1.06- |
| | | | | 1.02 (m, 2H), 0.99 (s, 3H). |
| 776 | *** | ** | 532.40 | 1H-NMR (400 MHz, DMSO-d6) δ 9.02 (d, J = 0.6 Hz, |
| | | | | 1H), 8.58 (s, 1H), 8.22 (s, 1H), 7.99 (d, J = 0.6 Hz, 1H), |
| | | | | 7.45 (td, J = 7.8, 1.8 Hz, 1H), 7.39-7.30 (m, 1H), 7.23- |
| | | | | 7.14 (m, 2H), 5.34 (s, 1H), 4.88 (s, 1H), 3.90 (tt, J = 7.5, |
| | | | | 3.9 Hz, 1H), 3.63 (s, 2H), 3.31-3.20 (m, 4H), 2.65- |
| | | | | 2.55 (m, 4H), 1.55 (s, 3H), 1.15-1.11 (m, 2H), 1.09 (s, |
| | | | | 3H), 1.06-1.01 (m, 2H), 0.99 (s, 3H). |
| 777 | *** | *** | 525.30 | 1H NMR (400 MHz, DMSO-d6): δ 9.05 (s, 1H), 9.02 (s, |
| | | | | 1H), 8.47 (s, 1H), 7.87 (s, 1H), 7.34 (q, J = 8.7 Hz, 1H), |
| | | | | 7.24 (td, J = 10.1, 2.5 Hz, 1H), 7.08 (tt, J = 6.6, 3.2 Hz, |
| | | | | 1H), 6.36 (s, 1H), 6.27 (s, 1H), 6.23 (s, 1H), 5.53 (d, J = |
| | | | | 5.3 Hz, 1H), 4.70 (s, 1H), 4.56 (d, J = 5.2 Hz, 1H), 3.35- |
| | | | | 3.27 (m, 4H), 2.57 (d, J = 7.0 Hz, 4H), 1.07 (d, J = 5.7 |
| | | | | Hz, 6H). |
| 778 | *** | ** | 543.30 | 1H-NMR (400 MHz, DMSO-d6) δ 9.10 (s, 1H), 8.98 (s, |
| | | | | 1H), 8.48 (s, 1H), 7.95 (t, J = 58.6 Hz, 1H), 7.73 (s, 1H), |
| | | | | 7.34 (dd, J = 15.5, 8.6 Hz, 1H), 7.25 (td, J = 10.1, 2.5 Hz, |
| | | | | 1H), 7.07 (td, J = 8.5, 2.3 Hz, 1H), 6.27 (s, 1H), 5.52 (d, |
| | | | | J = 5.4 Hz, 1H), 4.75 (s, 1H), 4.53 (d, J = 5.3 Hz, 1H), 3.53- |
| | | | | 3.37 (m, 4H), 2.58-2.42 (overlapped m, 4H), 1.08 (s, |
| | | | | 3H), 1.06 (s, 3H). |
| 779 | **** | ** | 543.40 | 1H-NMR (400 MHz, DMSO-d6) δ 9.12-9.08 (m, 1H), |
| | | | | 8.98 (s, 1H), 8.48 (s, 1H), 7.95 (t, J = 58.6 Hz, 1H), 7.73 |
| | | | | (s, 1H), 7.34 (dd, J = 15.5, 8.7 Hz, 1H), 7.25 (td, J = 10.2, |
| | | | | 2.6 Hz, 1H), 7.07 (td, J = 8.5, 2.3 Hz, 1H), 6.27 (s, 1H), |
| | | | | 5.52 (d, J = 5.4 Hz, 1H), 4.75 (s, 1H), 4.53 (d, J = 5.3 Hz, |
| | | | | 1H), 3.51-3.37 (m, 4H), 2.57-2.41 (overlapped m, |
| | | | | 4H), 1.08 (s, 3H), 1.06 (s, 3H). |
| 780 | **** | *** | 493.30 | 1H NMR (400 MHz, DMSO-d6): δ 9.02 (d, J = 10.5 Hz, |
| | | | | 1H), 8.62 (d, J = 21.3 Hz, 1H), 8.32 (d, J = 29.5 Hz, 1H), |
| | | | | 7.84 (d, J = 11.8 Hz, 1H), 7.40-7.31 (m, 1H), 7.25 (td, |
| | | | | J = 10.1, 2.5 Hz, 1H), 7.13-7.05 (m, 1H), 6.27 (d, J = 12.1 |
| | | | | Hz, 1H), 5.51 (t, J = 7.4 Hz, 1H), 4.73 (s, 1H), 4.55 (t, J = |
| | | | | 5.9 Hz, 1H), 3.41-3.23 (m, 4H), 2.68-2.55 (m, 4H), |
| | | | | 1.08 (d, J = 9.6 Hz, 6H). |
| 781 | *** | ** | 518.40 | 1H-NMR (400 MHz, DMSO-d6) δ 8.97 (d, J = 0.6 Hz, |
| | | | | 1H), 8.54 (s, 1H), 8.18 (s, 1H), 7.78 (s, 1H), 7.41 (td, J = |
| | | | | 7.8, 1.8 Hz, 1H), 7.33-7.25 (m, 1H), 7.19-7.11 (m, |
| | | | | 2H), 5.45 (d, J = 4.9 Hz, 1H), 4.63 (s, 1H), 4.49 (d, J = |
| | | | | 5.2 Hz, 1H), 3.91-3.80 (m, 1H), 3.59 (s, 2H), 3.28- |
| | | | | 3.16 (overlapping m, 4H), 2.62-2.50 (m, 4H), 1.12- |
| | | | | 1.06 (m, 2H), 1.02 (overlapping s, 3H), 1.00 (overlapping |
| | | | | s, 3H), 1.00-0.96 (m, 2H). |
| 782 | *** | ** | 543.40 | 1H-NMR (400 MHz, ACN-d3) δ 9.04 (d, J = 0.7 Hz, 1H), |
| | | | | 8.73 (s, 1H), 8.43 (d, J = 0.9 Hz, 1H), 7.98 (d, J = 0.7 Hz, |
| | | | | 1H), 7.17 (td, J = 9.3, 5.5 Hz, 1H), 7.01 (ddd, J = 11.6, |
| | | | | 8.8, 3.1 Hz, 1H), 6.94-6.87 (m, 1H), 6.16 (d, J = 52.5 |
| | | | | Hz, 1H), 4.55-4.47 (m, 2H), 4.08 (s, 1H), 3.66-3.57 |
| | | | | (m, 2H), 3.26-3.15 (m, 2H), 2.13-2.10 (overlapped m, |
| | | | | 2H), 1.99-1.95 (overlapped m, 2H), 1.62 (s, 3H), 1.16 |
| | | | | (s, 3H), 1.04 (s, 3H). |
| 783 | *** | ** | 543.40 | 1H-NMR (400 MHz, ACN-d3) δ 9.06-9.03 (m, 1H), |
| | | | | 8.73 (s, 1H), 8.43 (d, J = 0.7 Hz, 1H), 8.00-7.96 (m, |
| | | | | 1H), 7.17 (td, J = 9.3, 5.5 Hz, 1H), 7.01 (ddd, J = 11.6, |
| | | | | 8.8, 3.0 Hz, 1H), 6.90 (tdd, J = 9.1, 3.0, 1.8 Hz, 1H), 6.16 |
| | | | | (d, J = 52.6 Hz, 2H), 4.57-4.46 (m, 2H), 4.08 (s, 1H), |
| | | | | 3.67-3.56 (m, 2H), 3.26-3.14 (m, 2H), 2.13-2.10 |
| | | | | (overlapped m, 2H), 2.01-1.87 (overlapped m, 2H), 1.62 |
| | | | | (s, 3H), 1.16 (s, 3H), 1.04 (s, 3H). |
| 784 | **** | ** | 525.30 | 1H NMR (400 MHz, DMSO-d6): δ 9.08 (s, 1H), 8.88 (s, |
| | | | | 1H), 8.38 (s, 1H), 7.72 (s, 1H), 7.34 (dd, J = 15.5, 8.6 Hz, |
| | | | | 1H), 7.24 (td, J = 10.2, 2.6 Hz, 1H), 7.07 (td, J = 8.5, 2.3 |
| | | | | Hz, 1H), 6.34 (s, 1H), 6.27 (s, 1H), 6.21 (s, 1H), 5.52 (d, |
| | | | | J = 5.4 Hz, 1H), 4.77 (s, 1H), 4.53 (d, J = 5.3 Hz, 1H), 3.47 |
| | | | | (t, J = 11.6 Hz, 4H), 2.56-2.50 (m, 4H), 1.24 (d, J = 6.3 |
| | | | | Hz, 1H), 1.07 (d, J = 10.6 Hz, 6H). |
| 785 | **** | ** | 525.30 | 1H NMR (400 MHz, DMSO-d6): δ 9.07 (d, J = 11.6 Hz, |
| | | | | 1H), 8.87 (d, J = 9.8 Hz, 1H), 8.35 (d, J = 17.8 Hz, 1H), |
| | | | | 7.70 (d, J = 16.8 Hz, 1H), 7.39-7.29 (m, 1H), 7.27- |
| | | | | 7.19 (m, 1H), 7.13-7.03 (m, 1H), 6.34 (s, 1H), 6.27 (s, |
| | | | | 1H), 6.21 (s, 1H), 5.52 (s, 1H), 4.77 (s, 1H), 4.53 (s, 1H), |
| | | | | 3.50 (d, J = 4.3 Hz, 3H), 2.51 (tt, J = 3.4, 1.8 Hz, 4H), |
| | | | | 1.23 (s, 1H), 1.07 (d, J = 10.6 Hz, 6H). |
| 786 | **** | **** | 527.30 | 1H NMR (400 MHz, DMSO-d6): δ 9.03 (s, 1H), 8.94 (s, |
| | | | | 1H), 8.44 (d, J = 4.6 Hz, 1H), 7.85 (s, 1H), 7.35 (dd, J = |
| | | | | 15.4, 8.6 Hz, 1H), 7.19 (td, J = 10.1, 2.5 Hz, 1H), 7.04 |
| | | | | (td, J = 8.3, 2.3 Hz, 1H), 6.36 (s, 1H), 6.23 (s, 1H), 5.49 |
| | | | | (d, J = 5.3 Hz, 1H), 4.66 (s, 1H), 4.55 (d, J = 5.2 Hz, 1H), |
| | | | | 3.68 (d, J = 12.4 Hz, 2H), 2.78 (dd, J = 21.3, 10.4 Hz, |
| | | | | 2H), 2.70-2.59 (m, 2H), 1.80-1.59 (m, 3H), 1.58- |
| | | | | 1.43 (m, 2H), 1.06 (d, J = 6.7 Hz, 6H). |
| 787 | **** | **** | 527.30 | 1H NMR (400 MHz, DMSO) -d6): δ 9.03 (s, 1H), 8.94 (s, |
| | | | | 1H), 8.44 (s, 1H), 7.85 (s, 1H), 7.35 (q, J = 8.3 Hz, 1H), |
| | | | | 7.19 (td, J = 10.1, 2.5 Hz, 1H), 7.03 (tt, J = 11.1, 5.6 Hz, |
| | | | | 1H), 6.36 (s, 1H), 6.23 (s, 1H), 5.52 (d, J = 5.3 Hz, 1H), |
| | | | | 4.69 (s, 1H), 4.56 (t, J = 8.2 Hz, 1H), 3.68 (d, J = 12.5 Hz, |
| | | | | 2H), 2.78 (dd, J = 21.6, 10.7 Hz, 2H), 2.63 (d, J = 6.9 Hz, |
| | | | | 2H), 1.76-1.61 (m, 2H), 1.58-1.45 (m, 2H), 1.06 (d, |
| | | | | J = 6.5 Hz, 6H). |
| 788 | * | * | 445.20 | 1H NMR (400 MHz, DMSO-d6): δ 9.11 (s, 1H), 8.55 (d, |
| | | | | J = 5.7 Hz, 1H), 8.47 (s, 1H), 8.10 (s, 1H), 7.69-7.54 |
| | | | | (m, 4H), 7.27 (ddd, J = 8.1, 2.8, 1.4 Hz, 1H), 4.47 (s, 2H), |
| | | | | 3.94 (d, J = 8.9 Hz, 3H), 3.75 (t, J = 5.6 Hz, 2H), 2.82 (t, |
| | | | | J = 5.3 Hz, 2H). |
| 789 | ** | ** | 491.30 | 1H NMR (400 MHz, DMSO-d6): δ 8.26 (s, 1H), 7.97 (s, |
| | | | | 1H), 7.67 (dt, J = 15.0, 4.3 Hz, 1H), 7.58-7.54(m, 2H), |
| | | | | 7.28 (t, J = 8.5 Hz, 1H), 4.62 (d, J = 14.1 Hz, 2H), 4.18 (s, |
| | | | | 2H), 3.89 (s, 3H), 3.79 (d, J = 4.1 Hz, 2H), 3.51-3.42 |
| | | | | (m, 2H), 2.90 (t, J = 5.5 Hz, 1H), 2.77 (s, 3H), 2.12 (d, J = |
| | | | | 6.4 Hz, 3H). |
| 790 | *** | ** | 481.30 | 1H NMR (400 MHz, DMSO-d6): δ8.24 (s, 1H), 8.01 (s, |
| | | | | 1H), 7.38 (dd, J = 15.4, 8.6 Hz, 1H), 7.20-7.12 (m, 1H), |
| | | | | 7.05 (td, J = 8.4, 2.2 Hz, 1H), 4.59 (d, J = 15.6 Hz, 2H), |
| | | | | 3.90 (d, J = 1.5 Hz, 3H), 3.81-3.74 (m, 2H), 3.45 (d, J = |
| | | | | 11.2 Hz, 1H), 3.32 (s, 3H), 2.87 (t, J = 6.3 Hz, 2H), 2.73 |
| | | | | (t, J = 5.8 Hz, 1H), 2.61 (d, J = 12.2 Hz, 1H), 2.14-2.08 |
| | | | | (m, 3H), 1.92 (d, J = 10.3 Hz, 1H), 1.58 (d, J = 9.5 Hz, |
| | | | | 1H), 1.42 (dd, J = 12.3, 8.7 Hz, 1H), 1.33-1.25 (m, 2H), |
| | | | | 1.24 (d, J = 7.0 Hz, 3H). |
| 791 | *** | *** | 540.40 | 1H NMR (400 MHz, DMSO-d6): δ 8.99 (s, 1H), 7.61 (s, |
| | | | | 1H), 7.52 (dd, J = 10.3, 7.2 Hz, 1H), 7.44 (dd, J = 15.4, |
| | | | | 8.5 Hz, 1H), 7.19 (td, J = 10.1, 2.5 Hz, 1H), 7.05 (td, J = |
| | | | | 8.4, 2.6 Hz, 2H), 6.50 (s, 2H), 5.47 (d, J = 5.4 Hz, 1H), |
| | | | | 4.81 (s, 1H), 4.49 (d, J = 5.4 Hz, 1H), 3.51 (s, 2H), 3.38 |
| | | | | (s, 4H), 2.42 (s, 4H), 1.09 (s, 3H), 1.03 (s, 3H). |
| 792 | NA | NA | 490.40 | 1H-NMR (400 MHz, DMSO-d6) δ 9.18 (s, 1H), 8.93 (s, |
| | | | | 1H), 8.41 (s, 1H), 8.03 (s, 1H), 7.48 (dd, J = 15.5, 8.6 Hz, |
| | | | | 1H), 7.21 (merged td, J = 10.0, 2.5 Hz, 1H), 7.14 (merged |
| | | | | t, J = 56.0 Hz, 1H), 7.08 (merged td, 1H), 6.29 (d, J = |
| | | | | 52.2 Hz, 2H), 3.59 (s, 2H), 3.40-3.33 (m, 4H), 2.65- |
| | | | | 2.53 (m, 4H). (Note: CHF2 proton is merged with benzyl |
| | | | | protons, the integration stands 1H at δ 7.14). |
| 793 | ** | ** | 465.40 | 1H NMR (400 MHz, DMSO-d6) δ 9.13 (d, J = 0.6 Hz, |
| | | | | 1H), 8.90 (s, 1H), 8.48 (d, J = 0.6 Hz, 1H), 8.37 (s 1H), |
| | | | | 8.20 (formate s, 0.6H), 7.54-7.41 (m, 1H), 7.21 (td, J = |
| | | | | 10.0, 2.6 Hz, 1H), 7.07 (td, J = 8.4, 2.2 Hz, 1H), 6.28 (d, |
| | | | | J = 52.1 Hz, 2H), 3.57 (s, 2H), 3.48-3.38 (m, 4H), 2.62- |
| | | | | 2.53 (m, 4H). |
| 794 | *** | ** | 455.40 | 1H NMR (400 MHz, DMSO-d6) δ 8.89 (s, 1H), 8.65 (s, |
| | | | | 1H), 8.40 (s, 1H), 7.54-7.43 (m, 1H), 7.22 (td, J = 10.0, |
| | | | | 2.5 Hz, 1H), 7.08 (td, J = 8.5, 2.5 Hz, 1H), 6.65 (s, 1H), |
| | | | | 6.27 (d, J = 52.4 Hz, 2H), 6.18 (s, 2H), 3.60 (s, 2H), 3.15- |
| | | | | 3.05 (m, 4H), 2.64-2.57 (m, 4H). |
| 795 | *** | ** | 560.50 | 1H-NMR (400 MHz, CD3CN) δ 9.04 (d, J = 0.7 Hz, 1H), |
| | | | | 8.79 (s, 1H), 8.48 (s, 1H), 7.99 (d, J = 0.7 Hz, 1H), 7.47 |
| | | | | (t, J = 59.5 Hz, 1H), 7.27-7.19 (m, 1H), 7.11 (td, J = 9.2, |
| | | | | 4.6 Hz,, 1H), 7.07-6.99 (m, 1H), 4.57-4.41 (m, 1H), |
| | | | | 4.11 (bs, 1H), 3.63 (s, 2H), 3.39-3.29 (m, 4H), 2.72- |
| | | | | 2.57 (m, 4H), 1.62 (s, 3H), 1.16 (s, 3H), 1.04 (s, 3H). |
| 796 | *** | ** | 456.40 | 1H-NMR (400 MHz, DMSO-d6) δ 11.05 (bs, 1H), 8.92 |
| | | | | (s, 1H), 8.69 (s, 1H), 8.41 (s, 1H), 7.48 (dd, J = 15.4, 8.6 |
| | | | | Hz, 1H), 7.26-7.16 (m, 1H), 7.08 (td, J = 8.5, 2.2 Hz, |
| | | | | 1H), 6.84 (s, 1H), 6.28 (d, J = 52.3 Hz, 2H), 3.60 (s, 2H), |
| | | | | 3.23-3.10 (m, 4H), 2.65-2.55 (m, 4H). Plausibly |
| | | | | tautomers present |
| 797 | *** | ** | 546.50 | 1H NMR (400 MHz, CD3CN) δ 8.95 (s, 1H), 8.54 (s, |
| | | | | 1H), 8.30 (s, 1H), 7.44 (dt, J = 15.1, 7.5 Hz, 1H), 7.09- |
| | | | | 6.83 (m, 2H), 6.13 (d, J = 52.6 Hz, 2H), 4.85 (d, J = 1.9 |
| | | | | Hz, 1H), 3.59 (s, 2H), 3.51-3.36 (m, 4H), 2.67-2.51 |
| | | | | (m, 4H), 1.22 (s, 3H), 1.13 (s, 3H). OH peaks were not |
| | | | | observed. |
| 798 | *** | ** | 528.50 | 1H-NMR (400 MHz, DMSO-d6) δ 8.88 (s, 1H), 8.79 (s, |
| | | | | 1H), 8.37 (s, 1H), 7.44 (dd, J = 15.5, 8.5 Hz, 1H), 7.18 |
| | | | | (td, J = 10.0, 2.5 Hz, 1H), 7.09-6.99 (m, 2H), 6.25 (d, |
| | | | | J = 52.2 Hz, 2H), 4.63 (s, 1H), 4.07 (s, 2H), 3.56 (s, 2H), |
| | | | | 3.17 (m, 4H), 2.61-2.57 (m, 4H), 1.18 (s, 6H). |
| 799 | ** | ** | 496.30 | 1H NMR (400 MHz, DMSO) δ 9.01 (d, J = 0.4 Hz, 1H), |
| | | | | 8.57 (s, 1H), 8.31 (s, 1H), 7.83 (s, 1H), 7.49 (dd, J = 15.4, |
| | | | | 8.5 Hz, 1H), 7.22 (td, J = 10.2, 2.5 Hz, 1H), 7.08 (td, J = |
| | | | | 8.4, 2.1 Hz, 1H), 5.47 (d, J = 5.3 Hz, 1H), 4.67 (s, 1H), |
| | | | | 4.54 (d, J = 5.0 Hz, 1H), 3.59 (s, 2H), 3.27 (s, 4H), 2.60 |
| | | | | (s, 4H), 1.06 (d, J = 9.2 Hz, 6H). |
| 800 | *** | ** | 496.30 | 1H NMR (400 MHz, DMSO-d6) δ 9.01 (s, 1H), 8.56 (s, |
| | | | | 1H), 8.30 (s, 1H), 7.83 (s, 1H), 7.49 (dd, J = 15.4, 8.5 Hz, |
| | | | | 1H), 7.22 (td, J = 10.2, 2.5 Hz, 1H), 7.08 (td, J = 8.4, 2.2 |
| | | | | Hz, 1H), 5.47 (d, J = 5.2 Hz, 1H), 4.67 (s, 1H), 4.54 (d, |
| | | | | J = 4.9 Hz, 1H), 3.59 (s, 2H), 3.27 (s, 4H), 2.60 (s, 4H), |
| | | | | 1.06 (d, J = 9.2 Hz, 6H). |

¹
<0.1 uM = ****
≥0.1 uM to <1.0 uM = ***
≥1.0 uM to <30 uM = **
≥30 uM = *;
NA means the result of this compound is not available.

### Example 001. (Scheme 1). 1-(2-(4-(2,4-fluorophenoxy)piperidin-1-yl)-3-(1H-pyrazol-4-yl)-7,8-dihydropyrido[3,4-b]pyrazin-6(5H)-yl)ethan-1-one (Compound 32)

### Step a. 3-chloro-2-(4-(2,4-difluorophenoxy)piperidin-1-yl)pyrido[3,4-b]pyrazine (S2)

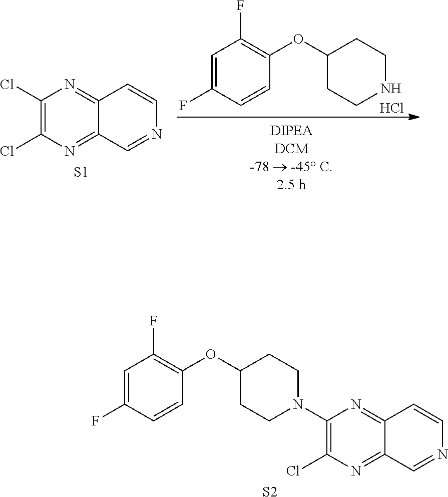

A round-bottom flask equipped with a stir bar was charged with 2,3-dichloropyrido[3,4-b]pyrazine (S1, 5.10 g, 20.3 mmol) in DCM (39.1 mL). The resulting mixture was cool down to −78° C., and DIPEA (7.06 mL, 40.5 mmol) was added followed by 4-(2,4-difluorophenoxy)piperidine (3.85 g, 18.1 mmol, portionwise). The mixture was allowed to warm up to −45° C. and stirred for 2.5 h. Afterwards, the reaction mixture was diluted with cold ethyl acetate (100 mL) and partitioned with cold water. The system was extracted with ethyl acetate (100 mL, 3×). The organic layer was washed with brine and dried over sodium sulfate. Following filtration and evaporation of volatiles, 2 was generated as a light yellow solid (6.27 g, 92%).

### Step b. 6-benzyl-3-chloro-2-(4-(2,4-difluorophenoxy)piperidin-1-yl)-5,6,7,8-tetrahydropyrido[3,4-b]pyrazine (S3)

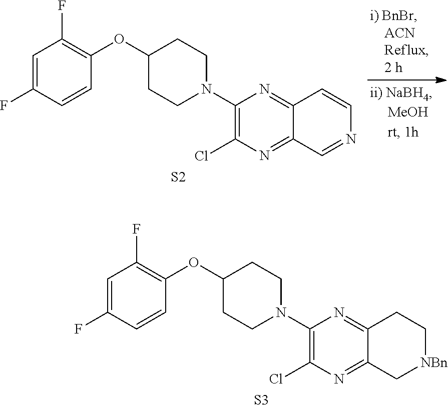 [SMILES: Fc1ccc(OC2CCN(c3nc4ccncc4nc3Cl)CC2)c(F)c1 ; "S2" ; Fc1ccc(OC2CCN(c3nc4c(nc3Cl)CN(Cc3ccccc3)CC4)CC2)c(F)c1 ; "S3"]

A 200 mL round-bottom flask equipped with a stir bar was charged with 2 (2.95 g, 7.83 mmol), followed by dilution in ACN (60.0 mL). Then, benzyl bromide (996 μL, 8.22 mmol) was added and the mixture heated to reflux for 2 h, where LCMS analysis indicated the consumption of the starting material. The solvents were evaporated under reduced pressure and the obtained crude was re-dissolved in MeOH (60.0 mL) and cooled to 0° C. Then, sodium borohydride (607 mg, 16.1 mmol) was added in portions. The mixture was stirred at 0° C. for 0.5 h and warmed to room temperature over 0.5 h. Afterwards, methanol was evaporated under reduced pressure and the obtained crude diluted with ethyl acetate, washed with water and the organic layer dried over sodium sulfate. The crude was purified by column chromatography over silica gel using hexanes/ethyl acetate as eluents to obtain S3 as a dark-cherry oil (1.50 g, 41%).

### Step c. 1-(3-chloro-2-(4-(2,4-difluorophenoxy)piperidin-1-yl)-7,8-dihydropyrido[3,4-b]pyrazin-6(5H)-yl)ethan-1-one (S4a)

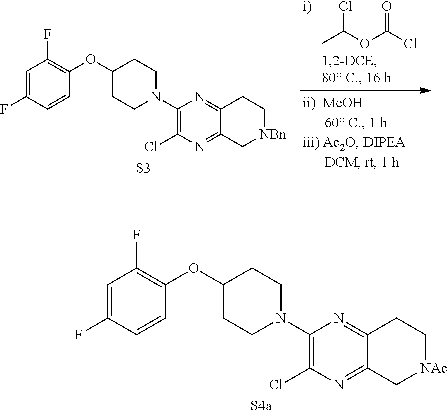

A 50 mL round-bottom flask equipped with a stir bar was charged with S3 (600 mg, 1.27 mmol) and diluted in 1,2-DCE (10.0 mL) before adding in 1-chloroethyl chloroformate (CECF, 300 μL, 2.79 mmol) at room temperature. Then, the system was heated to reflux overnight and the solvents were evaporated under reduced pressure. The obtained residue was re-dissolved in MeOH (10.0 mL) and heated at 60° C. for 1 h. Afterwards, the methanol was evaporated under reduced pressure and the obtained crude used as is in the acetylation step.

Reaction crude was then diluted in DCM (10.0 mL) followed by the addition of DIPEA (774 μL, 4.45 mmol) and acetic anhydride (252 μL, 2.67 mmol). The mixture was stirred at room temperature for 1 h. Then, it was transferred to a separatory funnel using ethyl acetate and water. The organic phase was washed with brine and dried over sodium sulfate. Reaction crude was purified by reverse phase column chromatography using ammonium formate/ACN as eluents to obtain S4a as a beige amorphous solid (220 mg, 41%).

### Step d. 1-(2-(4-(2,4-difluorophenoxy)piperidin-1-yl)-3-(1H-pyrazol-4-yl)-7,8-dihydropyrido[3,4-b]pyrazin-6(5H)-yl)ethan-1-one (Compound 32)

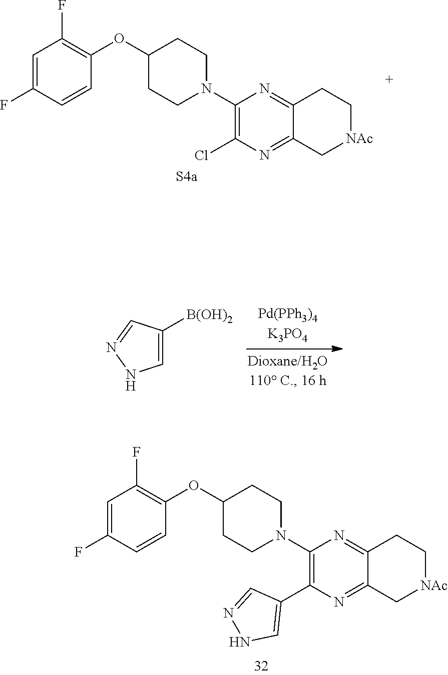

A microwave vial equipped with a stir bar was charged with &. (50.0 mg, 118 μmol), 1H-pyrazole-4-boronic acid (15.7 mg, 136 μmol), tetrakis(triphenylphosphine)palladium(0) (13.9 mg, 11.8 μmol) and potassium phosphate tribasic (76.8 mg, 355 μmol). The solids were diluted in a dioxane (300 μL)/water (100 μL) mixture and then degassed by nitrogen gas bubbling (5 min). Then, the container was sealed and the mixture heated to 110° C. overnight. Afterwards, the reaction mixture was filtered through Celite and the solvents evaporated under reduced pressure. Reaction crude was purified by reverse phase column chromatography using ammonium formate/ACN as eluents to obtain Compound 32 as a white amorphous solid (20 mg, 37%).

### Example 002. Scheme 1. 1-(2-(4-(2,4-difluorophenoxy)piperidin-1-yl)-3-(6-methoxypyridin-2-yl)-7,8-dihydropyrido[3,4-b]pyrazin-6(5H)-yl)ethan-1-one (Compound 86) and 6-(6-acetyl-2-(4-(2,4-difluorophenoxy)piperidin-1-yl)-5,6,7,8-tetrahydropyrido[3,4-b]pyrazin-3-yl)pyridin-2(1H)-one (Compound 33)

### Step e. 1-(2-(4-(2,4-difluorophenoxy)piperidin-1-yl)-3-(6-methoxypyridin-2-yl)-7,8-dihydropyrido[3,4-b]pyrazin-6(5H)-yl)ethan-1-one (Compound 86)

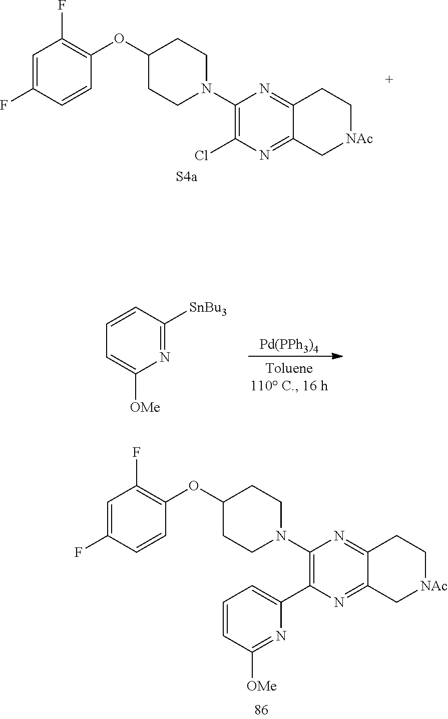

A microwave vial equipped with a stir bar was charged with S4a (105 mg, 248 μmol), 2-methoxy-6-(tributylstannyl)pyridine (109 mg, 261 μmol) and tetrakis(triphenylphosphine)-palladium(0) (29.3 mg, 24.8 μmol). The solids were diluted in toluene (1.05 mL) and then degassed by nitrogen gas bubbling (5 min). Then, the container was sealed and the mixture heated to 110° C. overnight. Afterwards, the reaction mixture was filtered through Celite and the solvents evaporated under reduced pressure. Reaction crude was purified by reverse phase column chromatography using ammonium formate/ACN as eluents to obtain Compound 86 as a yellow powder (70.0 mg, 57%).

### Step g. 6-(6-acetyl-2-(4-(2,4-difluorophenoxy)piperidin-1-yl)-5,6,7,8-tetrahydropyrido[3,4-b]pyrazin-3-yl)pyridin-2(1H)-one (Compound 33)

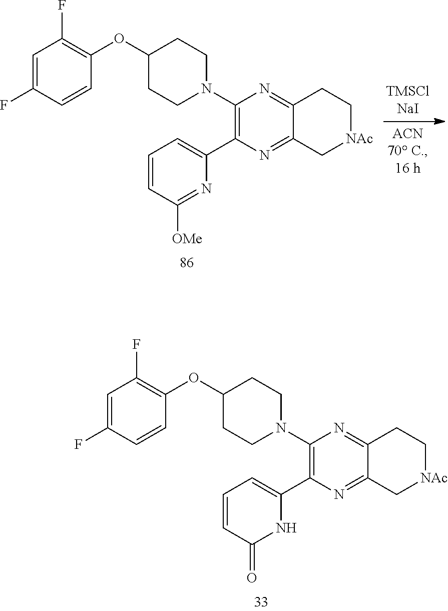

A microwave vial equipped with a stir bar was charged with sodium iodide (119 mg, 787 μmol), and diluted in ACN (1.00 mL). Vial was sealed and placed under nitrogen atmosphere. Chlorotrimethylsilane (101 μL, 787 μmol) was added dropwise at RT and mixture was stirred for 5 min. Then, the reaction mixture was cooled down to 0° C. and a solution of Compound 86 (60.0 mg, 121 μmol) in ACN (1.00 mL) was added slowly and warmed to room temperature. Afterwards, the system was heated to 70° C. overnight. The reaction was quenched by the addition of sodium thiosulphate saturated solution and the solvents were eliminated under reduced pressure. The mixture was transferred to a separatory funnel and extracted with ethyl acetate (×3). The organic phase was washed with brine and dried over sodium sulfate. Reaction crude was purified by reverse phase column chromatography using ammonium formate/ACN as eluents to obtain Compound 33 as a yellow powder (16.0 mg, 27%).

### Example 003. Scheme 1. 1-(2-(4-(2,4-difluorophenoxy)piperidin-1-yl)-3-(4-methoxypyridin-2-yl)-7,8-dihydropyrido[3,4-b]pyrazin-6(5H)-yl)ethan-1-one (Compound 7)

### Step e. 1-(2-(4-(2,4-difluorophenoxy)piperidin-1-yl)-3-(4-methoxypyridin-2-yl)-7,8-dihydropyrido[3,4-b]pyrazin-6(5H)-yl)ethan-1-one (Compound 7)

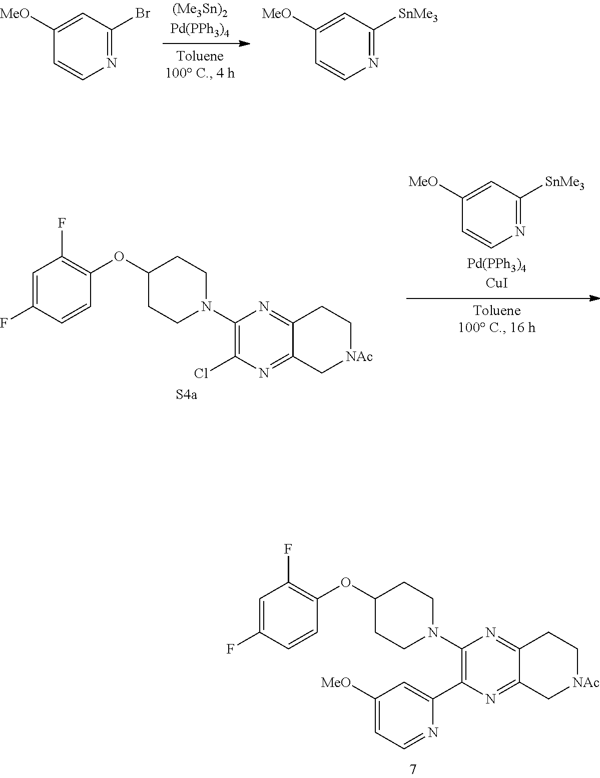

A reaction vial equipped with a stir bar was charged with 2-bromo-4-methoxypyridine (183 mg, 946 μmol), hexamethylditin (203 μL, 952 μmol) and tetrakis(triphenylphosphine)palladium(0) (110 mg, 94.6 μmol). The solids were diluted in toluene (3 mL) and then degassed by nitrogen gas bubbling (5 min). Then, the container was sealed and the mixture heated to 100° C. for 4 h. Afterwards, the system was cool down to room temperature and opened to add S4a (50.0 mg, 118 μmol), tetrakis(triphenylphosphine)palladium(0) (13.7 mg, 11.8 μmol) and copper iodide (25.3 mg, 130 μmol). The mixture was degassed by nitrogen gas bubbling (5 min), the container was sealed and the mixture heated to 100° C. overnight. Then, the reaction mixture was filtered through Celite and the solvents evaporated under reduced pressure. The crude product was directly purified by flash column chromatography (MeOH in Ethyl acetate as eluents) to obtain a solid. The resulting solid was further purified by reverse-phase chromatography using ammonium formate/ACN as eluents to obtain Compound 7 (28.4 mg, 47%) as yellow color solid.

### Example 004. Scheme 1. 1-(2-(4-(2,4-difluorophenoxy)piperidin-1-yl)-3-(4-methoxypyridin-2-yl)-7,8-dihydropyrido[3,4-b]pyrazin-6(5H)-yl)ethan-1-one (Compound 10)

### Step f. 1-(2-(4-(2,4-difluorophenoxy)piperidin-1-yl)-3-(4-methoxypyridin-2-yl)-7,8-dihydropyrido[3,4-b]pyrazin-6(5H)-yl)ethan-1-one (Compound 10)

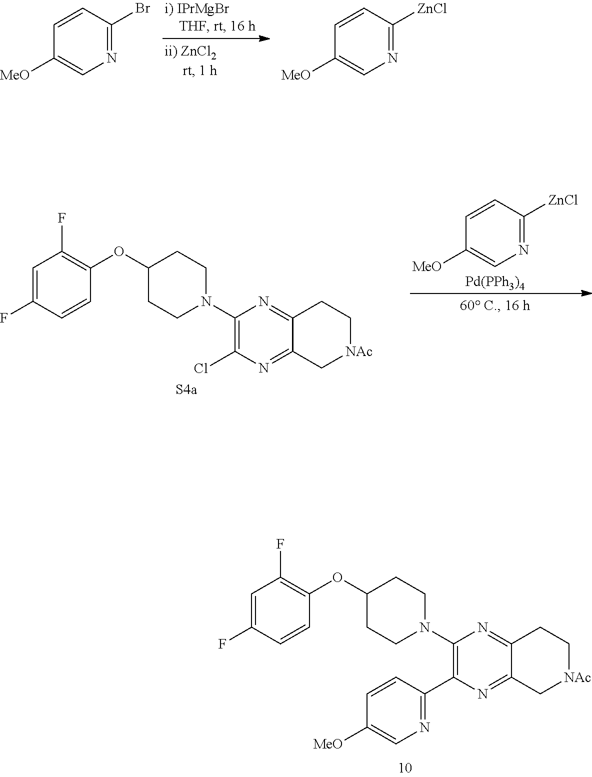

A 5 mL round bottom flask under nitrogen atmosphere was charged with 2-bromo-5-methoxypyridine (103 mg, 532 μmol), Isopropylmagnesium bromide solution (709 μL, 532 μmol) and THF (0.5 mL). The reaction mixture was stirred at room temperature for 16 hours. Then, solution of zinc chloride (1 M in Et₂O, 532 μL, 532 μmol) was added to the mixture and stirred at room temperature for 1 hour. The reaction system was opened to quickly add S4a (75.0 mg, 177 μmol) and tetrakis(triphenylphosphine)palladium(0) (20.5 mg, 17.7 μmol) at room temperature. The round bottom flask was sealed and the mixture degassed by nitrogen gas bubbling for 5 minutes at room temperature. Then, the system was heated to 60° C. for 16 hours. The crude product was directly purified by flash column chromatography (100% ethyl acetate, isocratic elution) to obtain a solid. The resulting solid was further purified by reverse-phase chromatography using ammonium formate/ACN as eluents to provide Compound 10 (20.0 mg, 23%) as yellow solid.

### Example 005. Scheme 1. 1-(2-(4-(2,4-difluorophenoxy)piperidin-1-yl)-3-(1H-imidazol-2-yl)-7,8-dihydropyrido[3,4-b]pyrazin-6(5H)-yl)ethan-1-one (Compound 40)

### Step g. 1-(2-(4-(2,4-difluorophenoxy)piperidin-1-yl)-3-(1H-imidazol-2-yl)-7,8-dihydropyrido[3,4-b]pyrazin-6(5H)-yl)ethan-1-one

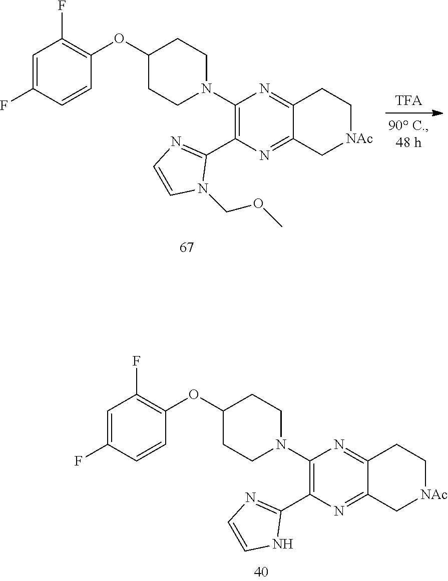

In a reaction vial (20 mL), Compound 67 (43.0 mg, 86.3 μmol) was dissolved in TFA (4.00 mL) at room temperature. The reaction mixture was heated to 90° C. for 48 h. After completion (indicated by LCMS), TFA was evaporated under reduced pressure and the obtained crude residue was directly purified by reverse phase column chromatography using ammonium formate/ACN as eluents to obtain Compound 40 as a yellow powder (17 mg, 43%).

### Example 006. Scheme 1. 1-(2-(4-(2,4-difluorophenoxy)piperidin-1-yl)-3-(5,6,7,8-tetrahydroimidazo[1,2-a]pyrazin-3-yl)-7,8-dihydropyrido[3,4-b]pyrazin-6(5)-yl)ethan-1-one (Compound 24)

### Step d. tert-butyl 3-(6-acetyl-2-(4-(2,4-difluorophenoxy)piperidin-1-yl)-5,6,7,8-tetrahydropyrido[3,4-b]pyrazin-3-yl)-5,6-dihydroimidazo[1,2-a]pyrazine-7(8H)-carboxylate (S5a)

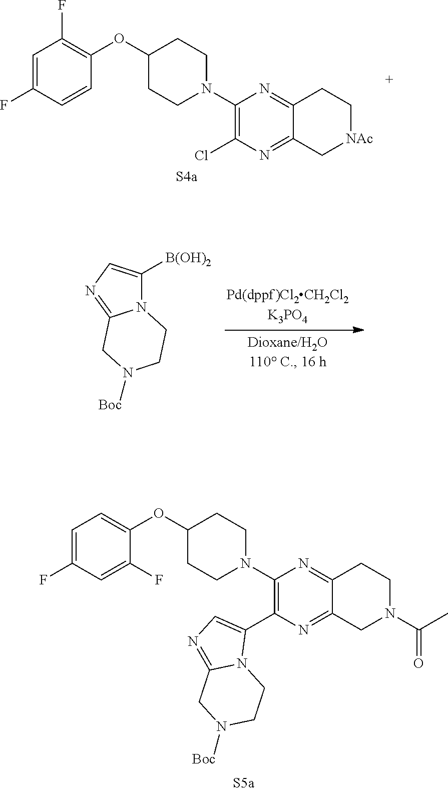

Compound S5a was synthesized following the procedure described in Example 001—Step d using Pd(dppf)Cl₂·CH₂Cl₂ as catalyst and was obtained as a light yellow viscous oil (76%).

### Step g. 1-(2-(4-(2,4-difluorophenoxy)piperidin-1-yl)-3-(5,6,7,8-tetrahydroimidazo[1,2-a]pyrazin-3-yl)-7,8-dihydropyrido[3,4-b]pyrazin-6(5H)-yl)ethan-1-one (Compound 24)

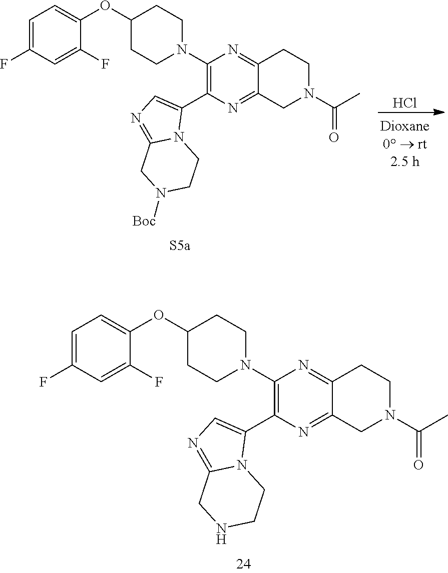 [SMILES: CC(=O)N1CCc2nc(N3CCC(Oc4ccc(F)cc4F)CC3)c(-c3cnc4n3CCN(C(=O)OC(C)(C)C)C4)nc2C1 ; Cl ; CC(=O)N1CCc2nc(N3CCC(Oc4ccc(F)cc4F)CC3)c(-c3cnc4n3CCNC4)nc2C1]

In a 25 mL round bottom flask, S5a (176 mg, 289 μmol) was dissolved in dioxane (2.00 mL). Then, the system was cooled down to 0° C. and solution of HCl (4 M in dioxane, 2.00 mL, 8.00 mmol) was added. The mixture was let warm to room temperature and stirred until completion (2.5 h). Afterwards, the solvent was removed and residue was washed with diethyl ether to get the hydrochloride salt of the desired product as a yellow solid (151 mg, 94%), which it was used in subsequent steps. A fraction of the obtained solid was purified by reverse phase column chromatography using ammonium bicarbonate/ACN as eluents to obtain Compound 24 as a yellow powder.

### Example 007. Scheme 1. 1-(3-(6-acetyl-2-(4-(2,4-difluorophenoxy)piperidin-1-yl)-5,6,7,8-tetrahydropyrido[3,4-b]pyrazin-3-yl)-5,6-dihydroimidazo[1,2-a]pyrazin-7(8H)-yl)ethan-1-one (Compound 17)

### Step g. 1-(3-(6-acetyl-2-(4-(2,4-difluorophenoxy)piperidin-1-yl)-5,6,7,8-tetrahydropyrido[3,4-b]pyrazin-3-yl)-5,6-dihydroimidazo[1,2-a]pyrazin-7(8H)-yl)ethan-1-one (Compound 17)

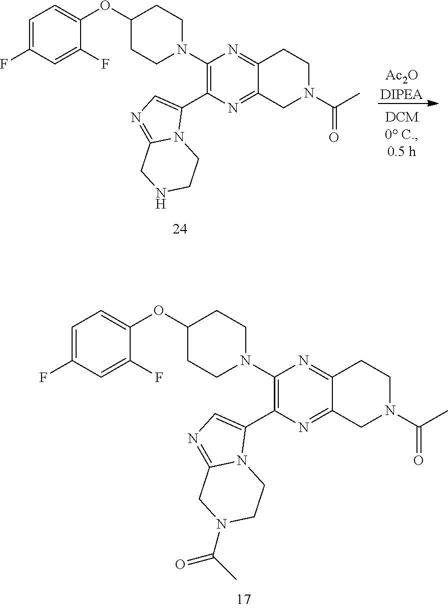

In a 10 mL round bottom flask under nitrogen atmosphere, a solution of Compound 24 (HCl salt, 50.0 mg, 91.6 μmol) in DCM (1.67 mL) was cooled down to 0° C. Then, acetic anhydride (17.5 μL, 183 μmol) was added followed by dropwise addition of N,N-diisopropylethylamine (80.6 μL, 458 μmol). The reaction mixture was stirred at 0° C. until completion of reaction (indicated by LCMS analysis). Afterwards, the solvent was evaporated and the crude directly purified by reverse phase column chromatography using ammonium bicarbonate/ACN as eluents to obtain Compound 17 as an off-white powder (25 mg, 49%).

### Example 008. Scheme 1. 1-(2-(4-(2,4-difluorophenoxy)piperidin-1-yl)-3-(7-methyl-5,6,7,8-tetrahydroimidazo[1,2-a]pyrazin-3-yl)-7,8-dihydropyrido[3,4-b]pyrazin-6(5H)-yl)ethan-1-one (Compound 90)

### Step g. 1-(2-(4-(2,4-difluorophenoxy)piperidin-1-yl)-3-(7-methyl-5,6,7,8-tetrahydroimidazo[1,2-a]pyrazin-3-yl)-7,8-dihydropyrido[3,4-b]pyrazin-6(5H)-yl)ethan-1-one (Compound 90)

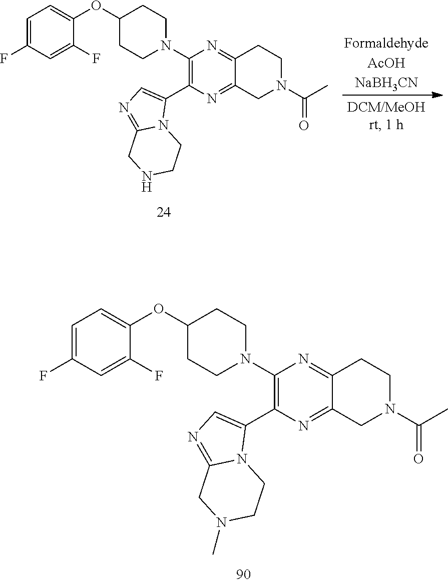 [SMILES: CC(=O)N1CCc2nc(N3CCC(Oc4ccc(F)cc4F)CC3)c(-c3cnc4n3CCNC4)nc2C1 ; "FormaldehydeAcOHNaBH3CN" ; CC(=O)N1CCc2nc(N3CCC(Oc4ccc(F)cc4F)CC3)c(-c3cnc4n3CCN(C)C4)nc2C1]

In a 10 mL round bottom flask, Compound 24 (HCl salt, 50.0 mg, 91.6 μmol) was dissolved in a mixture of DCM (933 μL) and MeOH (933 μL). Then, formaldehyde solution (37% in water, 29.2 μL, 366 μmol), acetic acid (52.4 μL, 916 μmol) and sodium cyanoborohydride (16.8 μL, 275 μmol) were added. The reaction mixture was stirred at room temperature until completion (indicated by LCMS analysis). Afterwards, the solvents were evaporated and the crude directly purified by reverse phase column chromatography using ammonium formate/ACN as eluents to obtain Compound 90 as a white powder (24 mg, 49%).

### Example 009. Scheme 1. 1-(3-(1-acetyl-1,2,3,6-tetrahydropyridin-4-yl)-2-(4-(2,4-difluorophenoxy)piperidin-1-yl)-7,8-dihydropyrido[3,4-b]pyrazin-6(5H)-yl)ethan-1-one (Compound 69) and 1-(4-(6-acetyl-2-(4-(2,4-difluorophenoxy)piperidin-1-yl)-5,6,7,8-tetrahydropyrido[3,4-b]pyrazin-3-yl)piperidin-1-yl)ethan-1-one (Compound 91, Compound 91)

### Step d. tert-butyl 4-(6-acetyl-2-(4-(2,4-difluorophenoxy)piperidin-1-yl)-5,6,7,8-tetrahydropyrido[3,4-b]pyrazin-3-yl)-3,6-dihydropyridine-1(2H)-carboxylate (S5b)

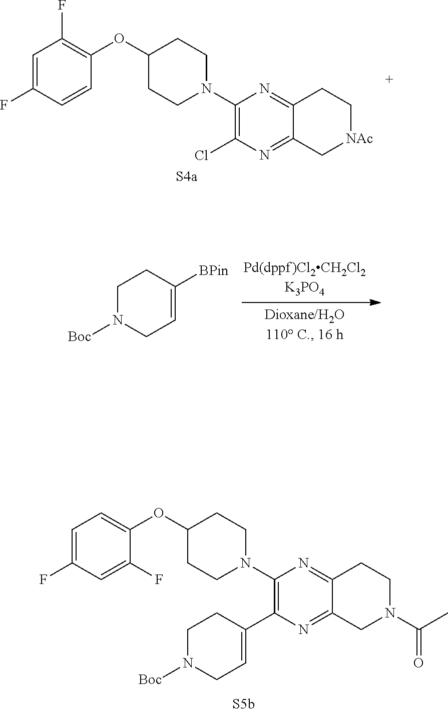

Compound S5b was synthesized following the procedure described in Example 001—Step d using Pd(dppf)Cl₂·CH₂Cl₂ as catalyst and was obtained as a yellow solid (98%).

### Step g. 1-(3-(1-acetyl-1,2,3,6-tetrahydropyridin-4-yl)-2-(4-(2,4-difluorophenoxy)piperidin-1-yl)-7,8-dihydropyrido[3,4-b]pyrazin-6(5H)-yl)ethan-1-one (Compound 69)

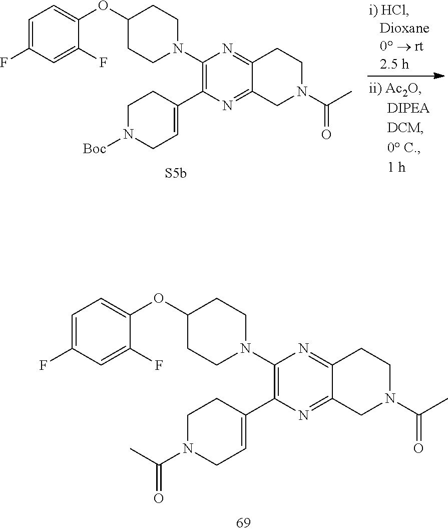 [SMILES: CC(=O)N1CCc2nc(N3CCC(Oc4ccc(F)cc4F)CC3)c(C3=CCN(C(=O)OC(C)(C)C)CC3)nc2C1 ; CC(=O)N1CC=C(c2nc3c(nc2N2CCC(Oc4ccc(F)cc4F)CC2)CCN(C(C)=O)C3)CC1]

Compound 69 was synthesized following the procedure described in Example 006—Step g followed by acylation using the procedure described in Example 007—Step g and was obtained as a white powder (45 mg, 64%, 2 steps).

### 1-(4-(6-acetyl-2-(4-(2,4-difluorophenoxy)piperidin-1-yl)-5,6,7,8-tetrahydropyrido[3,4-b]pyrazin-3-yl)piperidin-1-yl)ethan-1-one (Compound 91)

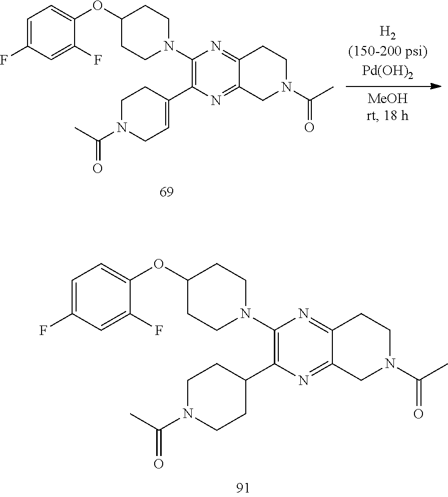 [SMILES: CC(=O)N1CC=C(c2nc3c(nc2N2CCC(Oc4ccc(F)cc4F)CC2)CCN(C(C)=O)C3)CC1 ; CC(=O)N1CCC(c2nc3c(nc2N2CCC(Oc4ccc(F)cc4F)CC2)CCN(C(C)=O)C3)CC1]

Using a high-pressure reactor equipped with a stir bar, Compound 69 (22.0 mg, 43.0 μmol) diluted in MeOH (2 mL) was transferred to the reaction container. Then, palladium hydroxide (20% wt, wet, 5 mg) was added followed by addition of acetic acid (1 drop). The reactor was sealed and placed under vacuum before back-filling with hydrogen and pressurizing to ˜150-200 psi. The reactor was set to stir at room temperature for 18 h (reaction completed as indicated by LCMS analysis). Afterwards, the reactor depressurised and opened, and the reaction mixture was filtered through Celite bed. The solvents were evaporated and the obtained crude was directly purified by reverse phase column chromatography using ammonium formate/ACN as eluents to obtain Compound 91 (20 mg, 91%) as a white powder.

### Example 010. Scheme 1. 2-(4-(2,4-difluorophenoxy)piperidin-1-yl)-6-(methylsulfonyl)-3-(1H-pyrazol-4-yl)-5,6,7,8-tetrahydropyrido[3,4-b]pyrazine (Compound 70)

### Step c. 3-chloro-2-(4-(2,4-difluorophenoxy)piperidin-1-yl)-6-(methylsulfonyl)-5,6,7,8-tetrahydropyrido[3,4-b]pyrazine (S4b)

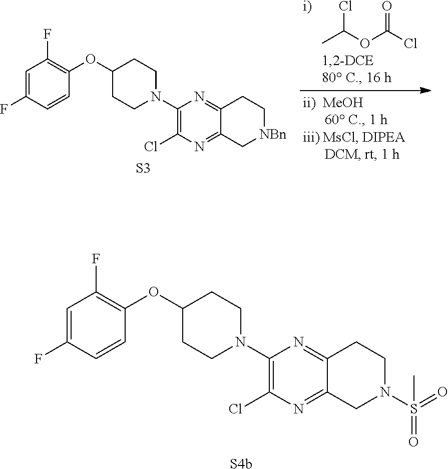

A 50 mL round-bottom flask equipped with a stir bar was charged with S3 (335 mg, 711 μmol) and diluted in 1,2-DCE (10.0 mL) before adding in 1-chloroethyl chloroformate (CECF, 168 μL, 1.56 mmol) at room temperature. Then, the system was heated to reflux overnight and the solvents were evaporated under reduced pressure. The obtained residue was re-dissolved in MeOH (5.0 mL) and heated at 60° C. for 1 h. Afterwards, methanol was evaporated under reduced pressure and the obtained crude used as is in the next step.

Reaction crude was then diluted in DCM (5.0 mL) followed by the addition of DIPEA (625 μL, 3.55 mmol) and methanesulfonyl chloride (166 μL, 2.13 mmol). The mixture was stirred overnight at room temperature. Then, it was transferred to a separatory funnel using ethyl acetate and water. The organic phase was washed with brine and dried over sodium sulfate. Reaction crude was purified by column chromatography using hexanes/ethyl acetate as eluents to obtain S4b as a brown solid (282 mg, 86%).

### Step d. 2-(4-(2,4-difluorophenoxy)piperidin-1-yl)-6-(methylsulfonyl)-3-(1H-pyrazol-4-yl)-5,6,7,8-tetrahydropyrido[3,4-b]pyrazine (Compound 70)

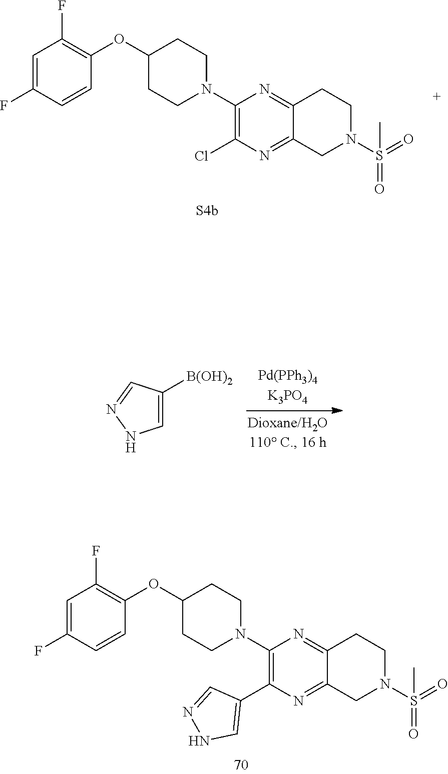 [SMILES: CS(=O)(=O)N1CCc2nc(N3CCC(Oc4ccc(F)cc4F)CC3)c(Cl)nc2C1 ; "S4b" ; OB(O)c1cn[nH]c1 ; CS(=O)(=O)N1CCc2nc(N3CCC(Oc4ccc(F)cc4F)CC3)c(-c3cn[nH]c3)nc2C1]

Compound 70 was synthesized following the procedure described in Example 001—Step d and was obtained as a white powder (46.0 mg, 33%).

### Example 011. Scheme 1. 2-(4-(2,4-difluorophenoxy)piperidin-1-yl)-3-(6-methoxypyridin-2-yl)-6-(methylsulfonyl)-5,6,7,8-tetrahydropyrido[3,4-b]pyrazine (Compound 59)

### Step d. 2-(4-(2,4-difluorophenoxy)piperidin-1-yl)-3-(6-methoxypyridin-2-yl)-6-(methylsulfonyl)-5,6,7,8-tetrahydropyrido[3,4-b]pyrazine (Compound 59)

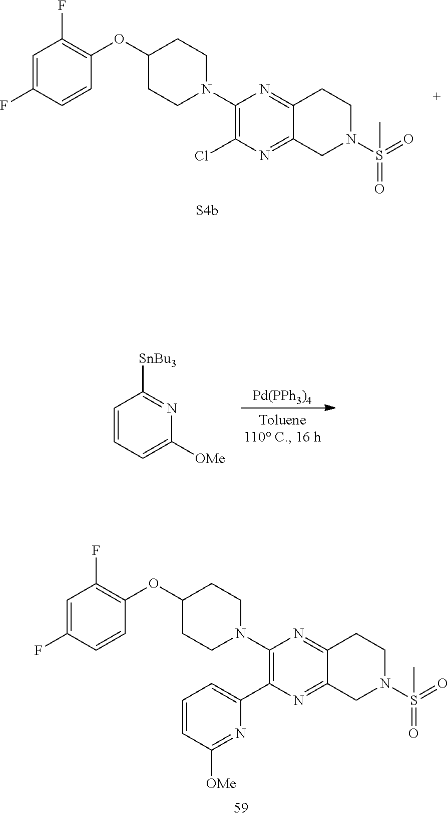

Compound 59 was synthesized following the procedure described in Example 002—Step e and was obtained as a light yellow powder (101 mg, 58%).

### Example 012. Scheme 2. 1-(3-(1H-pyrazol-4-yl)pyrido[3,4-b]pyrazin-2-yl)-N-(2,4-difluorophenyl)piperidin-4-anine (Compound 35)

### Step a. tert-butyl 4-((2,4-difluorophenyl)amino)piperidine-1-carboxylate (S9a)

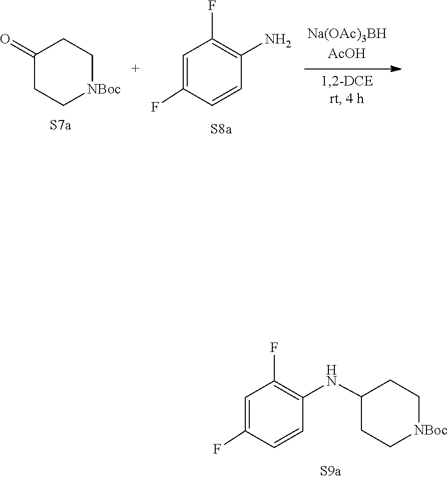 [SMILES: CC(C)(C)OC(=O)N1CCC(=O)CC1 ; Nc1ccc(F)cc1F ; CC(C)(C)OC(=O)N1CCC(Nc2ccc(F)cc2F)CC1]

A 25 mL round bottom flask equipped with a stir bar was charged with a solution of 2,4-difluoroaniline (S8a, 394 μL, 3.87 mmol) and 1-Boc-4-piperidone (S7a, 991 μL, 3.87 mmol) in DCE (5.00 mL). Then, acetic acid (448 μL, 7.75 mmol) was added and the system stirred at room temperature for 30 min. Next, sodium triacetoxyborohydride (1.64 g, 7.75 mmol) was added portionwise and stirred for 4 h at the same temperature. The reaction was quenched by the addition of 3 N NaOH (5.0 mL), extracted with ethyl acetate (×3), washed thoroughly with water (×3) and dried over sodium sulfate. Purification by flash chromatography using hexanes/ethyl acetate as eluents gives compound S9A (710 mg, 59%) as a yellow oil.

### Step b. 1-(3-chloropyrido[3,4-b]pyrazin-2-yl)-N-(2,4-difluorophenyl)piperidin-4-amine (S10a)

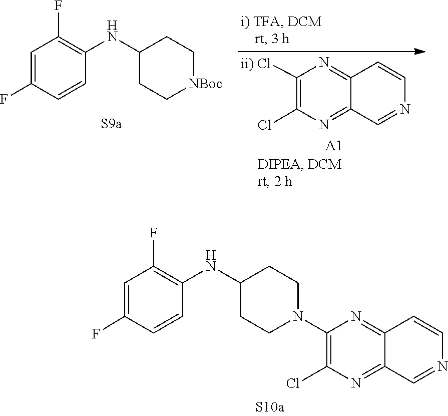

Step i. To a solution of amine S9a (400 mg, 1.28 mmol) in DCM (5.0 mL) was added trifluoroacetic acid (2.0 mL, 26.1 mmol) at room temperature. The system was stirred for 3 h at same temperature. After the full consumption of starting material, the excess TFA was removed under vacuum and azeotrope with DCM (3×5.0 mL) and dried further under vacuum.

Step ii. The obtained reaction crude was dissolved in DCM (10.0 mL) and cooled to 0° C. Then, DIPEA (1.23 mL, 7.09 mmol) was added. After 5 min, 2,3-dichloropyrido[3,4-b]pyrazine (S1, 333 mg, 1.41 mmol) was added and the mixture stirred for 2 h at room temperature. Then, the reaction mixture was concentrated under a vacuum to remove excess solvents and reagents. Afterwards, the obtained crude was directly loaded into a silica for purification using hexanes/ethyl acetate as eluents to give compound S10a (418 mg, 87%) as a yellow solid.

### Step d. 1-(3-(1H-pyrazol-4-yl)pyrido[3,4-b]pyrazin-2-yl)-N-(2,4-difluorophenyl)piperidin-4-amine (Compound 35)

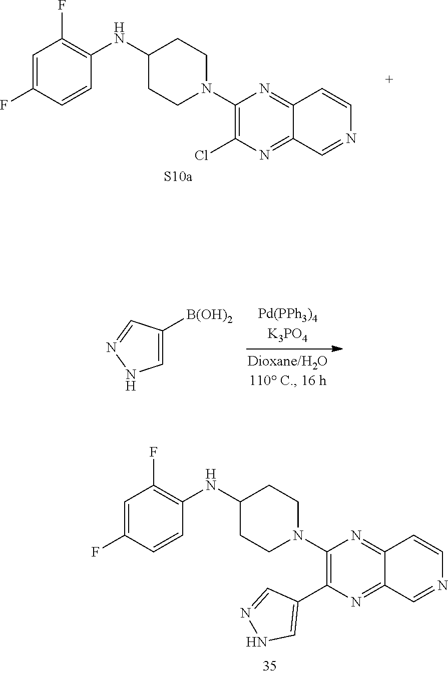

Compound 35 was synthesized following the procedure described in Example 001—Step d and was obtained as a light yellow powder (90.0 mg, 28%).

### Example 013. Scheme 2. 1-(3-(1H-pyrazol-4-yl)pyrido[3,4-b]pyrazin-2-yl)-N-(2,4-difluorophenyl)-N-methylpiperidin-4-amine (Compound 55)

### Step c. 1-(3-chloropyrido[3,4-b]pyrazin-2-yl)-N-(2,4-difluorophenyl)-N-methylpiperidin-4-amine (S10)

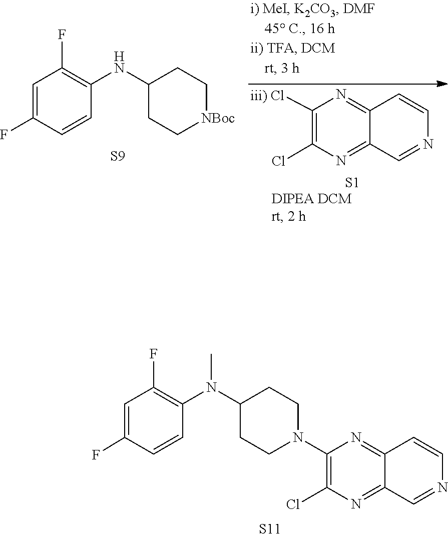

Step i. Using a 25 mL round bottom flask equipped with a stir bar, to a solution of amine S9 (290 mg, 928 μmol) in DMF (5.00 mL), iodomethane (117 μL, 1.86 mmol) and potassium carbonate (393 mg, 2.79 mmol) were added sequentially and the resulting mixture was heated at 45° C. overnight. Then, the reaction was diluted with water (10 mL), extracted with ethyl acetate and dried over sodium sulfate. The concentration of organic layer gave the desired intermediate (230 mg, 76%) with sufficient purity for next step.

Step ii. The solid crude obtained was dissolved in DCM (5.0 mL) and trifluoroacetic acid (2.0 mL, 26.1 mmol) was added at room temperature. The system was stirred for 3 h at same temperature. After the full consumption of starting material, the excess TFA was removed under vacuum and azeotrope with DCM (3×5.0 mL) and dried further under vacuum.

Step iii. The obtained reaction crude was dissolved in DCM (10.0 mL) and cooled to 0° C. Then, DIPEA (613 μL, 3.52 mmol) was added. After 5 min, 2,3-dichloropyrido[3,4-b]pyrazine (S1, 183 mg, 776 μmol) was added and the mixture stirred for 2 h at room temperature. Then, the reaction mixture was concentrated under a vacuum to remove excess solvents and reagents. Afterwards, the obtained crude was directly loaded into a silica for purification using hexanes/ethyl acetate as eluents to give compound S11 (210 mg, 76%) as a yellow solid.

### Step d. 1-(3-(1H-pyrazol-4-yl)pyrido[3,4-b]pyrazin-2-yl)-N-(2,4-difluorophenyl)-N-methylpiperidin-4-amine (Compound 55)

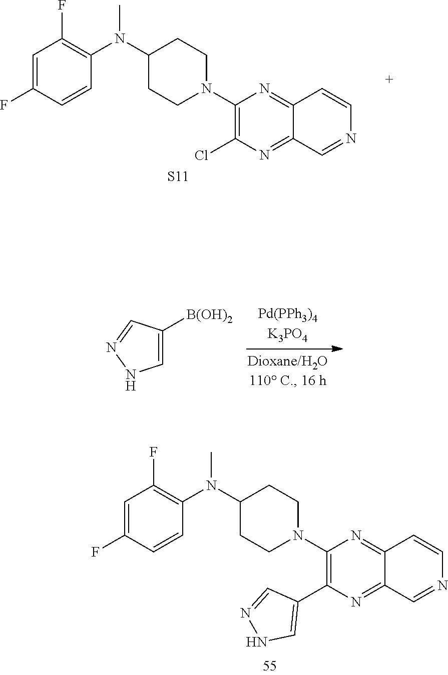 [SMILES: CN(c1ccc(F)cc1F)C1CCN(c2nc3ccncc3nc2Cl)CC1 ; "S11" ; OB(O)c1cn[nH]c1 ; CN(c1ccc(F)cc1F)C1CCN(c2nc3ccncc3nc2-c2cn[nH]c2)CC1]

Compound 55 was synthesized following the procedure described in Example 001—Step d and was obtained as a light yellow powder (38.5 mg, 18%).

### Example 014. Scheme 2. 2-(4-(2,4-difluorophenoxy)-4-(methoxymethyl)piperidin-1-yl)-3-(1H-pyrazol-4-yl)pyrido[3,4-b]pyrazine (Compound 63)

### Step a. 1-(tert-butoxycarbonyl)-4-(2,4-difluorophenoxy)piperidine-4-carboxylic Acid (S9b)

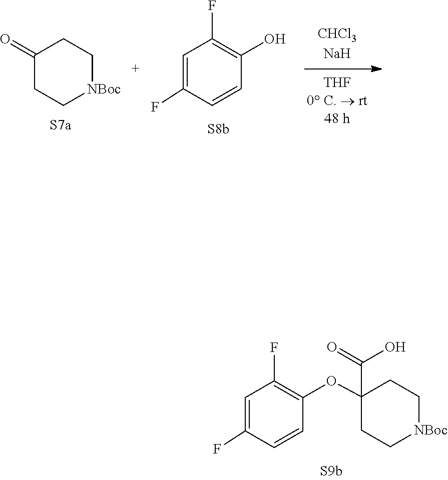 [SMILES: CC(C)(C)OC(=O)N1CCC(=O)CC1 ; Oc1ccc(F)cc1F ; CC(C)(C)OC(=O)N1CCC(Oc2ccc(F)cc2F)(C(=O)O)CC1]

Following procedure reported in WO2013/10453, 2013, A1. In a 250 mL round bottom flask, sodium hydride (60% in mineral oil, 2.15 g, 53.6 mmol) was added to a stirred solution of 2,4-difluorophenol (S8b, 2.00 g, 15.1 mmol) in anhydrous THF (60.0 mL) at room temperature; the mixture was stirred for 3 h. Then, 1-Boc-4-piperidone (S7a, 9.19 g, 45.2 mmol) was added. The mixture was cooled down to 0° C. and anhydrous chloroform (5.64 mL, 69.8 mmol) was added dropwise. The reaction mixture was stirred at 0° C. for 1 h and then it was allowed to warm room temperature, where it was stirred for 48 h (conversion stalls, indicated by LCMS analysis). Afterwards, the reaction mixture was slowly acidified using 1N HCl until pH 5 where solid precipitation occurs. The solid obtained was filtered and washed with ethyl acetate. Then, the solid was dissolved DCM, transferred to a separatory funnel and washed with 1N aqueous HCl. The organic phase was dried over sodium sulfate. The concentration of organic layer gave the desired intermediate S9b (2.10 g, 39%) with sufficient purity for next step.

### tert-butyl 4-(2,4-difluorophenoxy)-4-(hydroxymethyl)piperidine-1-carboxylate (S9)

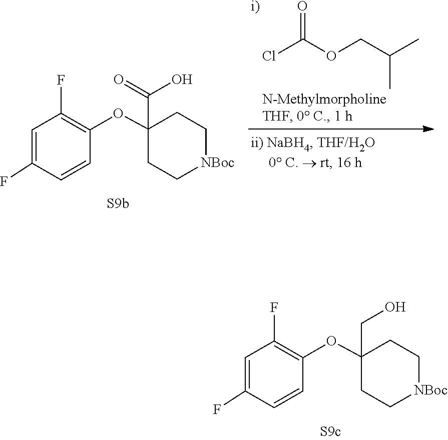

Step i. In a 100 mL round bottom flask, S9b (2.10 g, 5.88 mmol) was dissolved in anhydrous THF (29.4 mL) and the system was cooled down to 0° C. Then, 4-methylmorpholine (646 μL, 5.88 mmol) and isobutyl chloroformate (778 μL, 5.88 mmol) were sequentially added and the reaction mixture stirred for 1 h, keeping the temperature at 0° C.

Step ii. In a separate 50 mL round bottom flask, sodium borohydride (926 mg, 23.5 mmol) was dissolved in a mixture of THF (14.7 mL) and Water (14.7 mL) and the system cooled down to 0° C. Then, reaction mixture from Step 1 was passed filtered through a syringe with cotton filter and the filtrate was added dropwise to the flask containing the sodium borohydride solution at 0° C. This reaction mixture was slowly warmed to room temperature and stirred for 18 h. The reaction mixture was quenched by addition of saturated NH₄Cl solution and it was extracted with ethyl acetate (3×). The combined organic layers were washed with brine, dried over sodium sulfate and evaporated under reduced pressure. The obtained crude was purified over silica column using hexanes/EtOAc as eluents to give S9c (0.910 g, 45%) as a colorless oil.

### tert-butyl 4-(2,4-difluorophenoxy)-4-(methoxymethyl)piperidine-1-carboxylate (S9d)

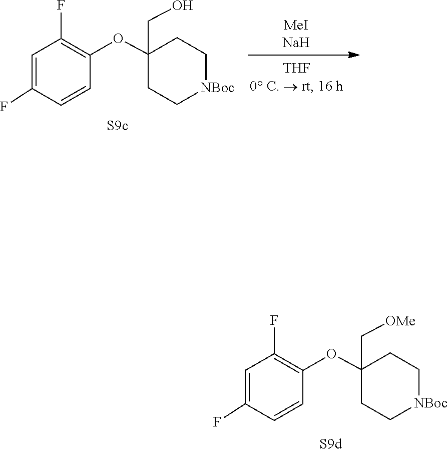 [SMILES: CC(C)(C)OC(=O)N1CCC(CO)(Oc2ccc(F)cc2F)CC1 ; COCC1(Oc2ccc(F)cc2F)CCN(C(=O)OC(C)(C)C)CC1]

In a 50 mL round bottom flask, to a solution of S9c (0.990 g, 2.88 mmol) in anhydrous THF (10 mL) at 0° C., sodium hydride (138 mg, 5.77 mmol) was added portion-wise. After addition, the mixture was stirred at 0° C. for 0.5 h, and then, methyl iodide (544 μL, 8.65 mmol) was added dropwise. The resulting mixture was stirred at room temperature for 16 h. The reaction mixture was quenched by addition of water and it was extracted with ethyl acetate (3×). The combined organic layers were washed with brine, dried over sodium sulfate and evaporated under reduced pressure. The obtained crude was purified over silica column using hexanes/EtOAc as eluents to give S9d (0.750 g, 73%) as a colorless oil.

### Step b. 3-chloro-2-(4-(2,4-difluoro henoxy)-4-(methoxymethyl)piperidin-1-yl)pyrido[3,4-b]pyrazine (S10b)

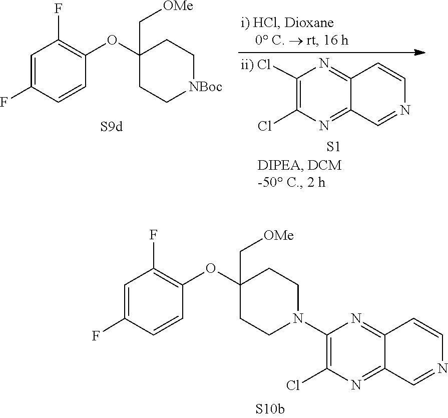

Step i. To a solution of amine S9d (0.750 g, 2.10 mmol) in dioxane (4.20 mL) was added solution of HCl (4 M in dioxane, 5.25 mL, 21.0 mmol) at 0° C. The system was let warm to room temperature and stirred for 16 h. After the full consumption of starting material (indicated by LCMS analysis), the solvents were removed and residue was washed with diethyl ether and dried under vacuum to get the hydrochloride salt of the desired intermediate as an off-white solid (0.550 g, 89%).

Step ii. The obtained amine (245 mg, 833 μmol) was mixed with 2,3-dichloropyrido[3,4-b]pyrazine (S1, 200 mg, 1.00 mmol). The solids were dissolved in DCM (5.55 mL) and cooled to −50° C. Then, DIPEA (1.23 mL, 7.09 mmol) was added dropwise and the mixture stirred for 2 h at −50° C. (full conversion indicated by LCMS analysis). Then, the reaction mixture was concentrated under a vacuum to remove excess solvents and reagents. Afterwards, the reaction mixture was quenched by addition of ice cold water and it was extracted with ethyl acetate (3×). The combined organic extracts were dried over sodium sulfate and evaporated under reduced pressure. The obtained crude was purified over silica column using hexanes/EtOAc as eluents to give S10b (72.0 mg, 21%) as a light yellow oil.

### Step c. 2-(4-(2,4-difluorophenoxy)-4-(methoxymethyl)piperidin-1-yl)-3-(1H-pyrazol-4-yl)pyrido[3,4-b]pyrazine (Compound 63)

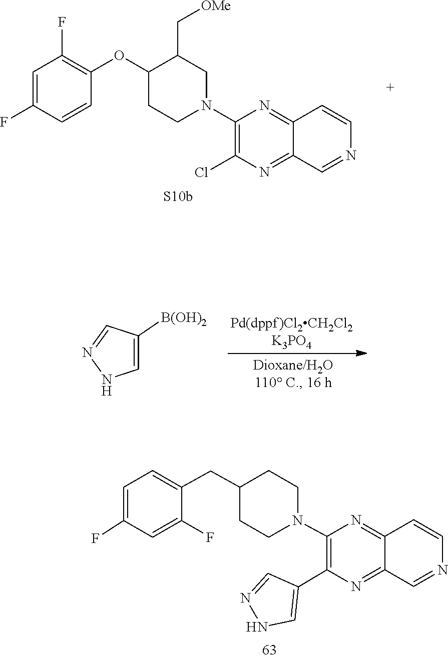 [SMILES: COCC1CN(c2nc3ccncc3nc2Cl)CCC1Oc1ccc(F)cc1F ; OB(O)c1cn[nH]c1 ; Fc1ccc(CC2CCN(c3nc4ccncc4nc3-c3cn[nH]c3)CC2)c(F)c1]

Compound 63 was synthesized following the procedure described in Example 001—Step d using Pd(dppf)Cl₂·CH₂Cl₂ as the catalyst and was obtained as a yellow solid (20.1 mg, 25%).

### Example 015. Scheme 2. 2-(6-(2,4-difluorophenoxy)-2-azaspiro[3.3]heptan-2-yl)-3-(1H-pyrazol-4-yl)pyrido[3,4-b]pyrazine (Compound 26)

### Step a. tert-butyl 6-(2,4-difluorophenoxy)-2-azaspiro[3.3]heptane-2-carboxylate (S9e)

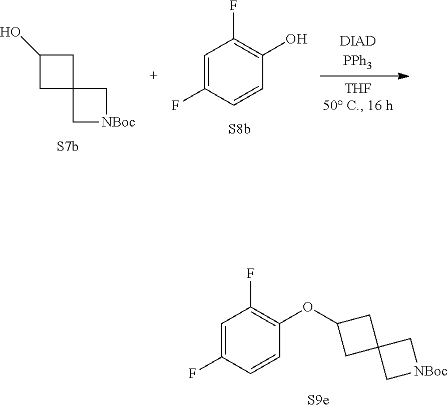 [SMILES: CC(C)(C)OC(=O)N1CC2(CC(O)C2)C1 ; Oc1ccc(F)cc1F ; CC(C)(C)OC(=O)N1CC2(CC(Oc3ccc(F)cc3F)C2)C1]

In a 50 mL round bottom flask under nitrogen atmosphere, diisopropyl-azodicarboxylate (DIAD, 940 μL, 4.68 mmol) was dissolved in THF (6.0 mL). Then, triphenylphosphine (1.30 g, 4.90 mmol) and 2,4-difluorophenol (S8b, 434 μL, 4.45 mmol) were added sequentially and the mixture stirred for 15 min at room temperature. Then, tert-Butyl 6-hydroxy-2-azaspiro[3.3]heptane-2-carboxylate (S7b, 1.00 g, 4.45 mmol) in THF (4.0 mL) was added and the mixture heated to 50° C. and stirred for 16 h. Afterwards, the system was cooled and quenched by adding 3 N NaOH, extracted with EtOAc (3×) and washed thoroughly with water (2×). The combined organic extracts were dried over sodium sulfate and evaporated under reduced pressure. The obtained crude was purified over silica column using hexanes/EtOAc as eluents to give S9e (1.20 g, 83%) as an off-white solid.

### Step b. 3-chloro-2-(6-(2,4-difluoro phenoxy)-2-azaspiro[3.3]heptan-2-yl)pyrido[3,4-b]pyrazine (S10c)

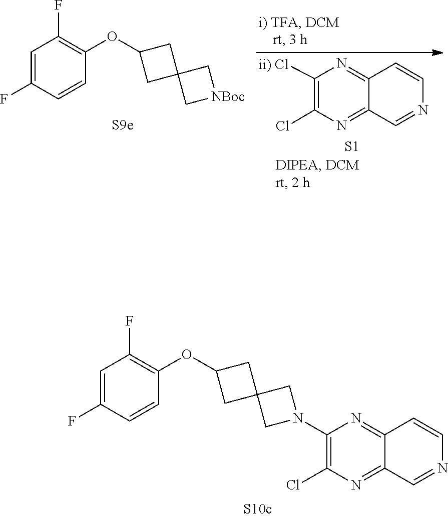

S9e was deprotected following the procedure from Example 012—Step b. S10c was synthesized from the deprotected s9e following the procedure described in Example 001—Step a. S10c was obtained as a yellow solid (302 mg, 40%).

### Step c. 2-(6-(2,4-difluorophenoxy)-2-azaspiro[3.3]heptan-2-yl)-3-(1H-pyrazol-4-yl)pyrido[3,4-b]pyrazine (Compound 26)

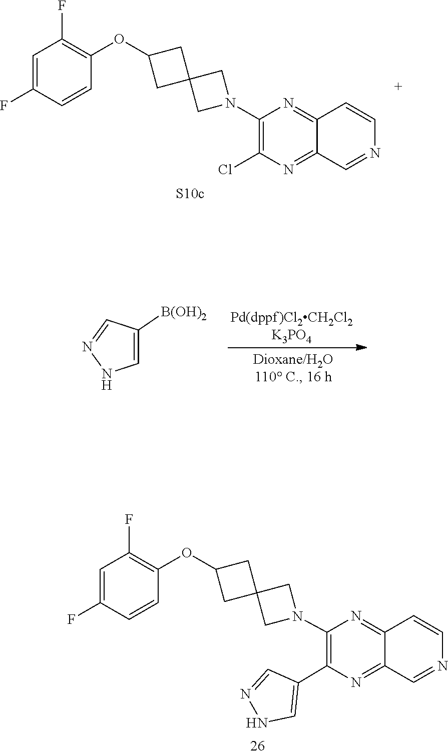 [SMILES: Fc1ccc(OC2CC3(C2)CN(c2nc4ccncc4nc2Cl)C3)c(F)c1 ; OB(O)c1cn[nH]c1 ; Fc1ccc(OC2CC3(C2)CN(c2nc4ccncc4nc2-c2cn[nH]c2)C3)c(F)c1]

Compound 26 was synthesized following the procedure described in Example 001—Step d using Pd(dppf)Cl₂·CH₂Cl₂ as catalyst and was obtained as an off-white powder (28.0 mg, 20%).

### Example 016. Scheme 2. 2-(6-(2,4-difluorophenoxy)-2-azaspiro[3.3]heptan-2-yl)-3-(6-methoxypyrazin-2-yl)pyrido[3,4-b]pyrazine (Compound 92)

### Step d. 2-(6-(2,4-difluorophenoxy)-2-azaspiro[3.3]heptan-2-yl)-3-(6-methoxypyrazin-2-yl)pyrido[3,4-b]pyrazine (Compound 92)

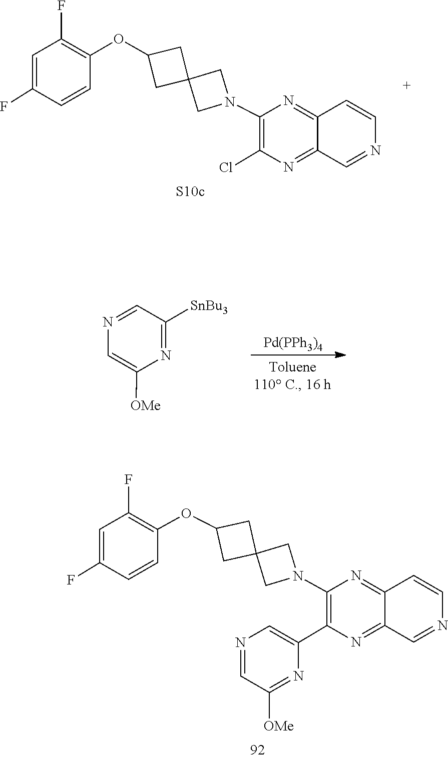

Compound 92 was synthesized following the procedure described in Example 002—Step e and was obtained as an off-white powder (56.2 mg, 30%).

### Example 017. Scheme 2. 4-(3-(1H-pyrazol-4-yl)pyrido[3,4-b]pyrazin-2-yl)-1-((2,4-difluorophenyl)imino)-1l6-thiomorpholine 1-oxide (Compound 58)

### Step a. tert-butyl 1-((2,4-difluorophenyl)imino)-1l6-thiomorpholine-4-carboxylate 1-oxide (S9f)

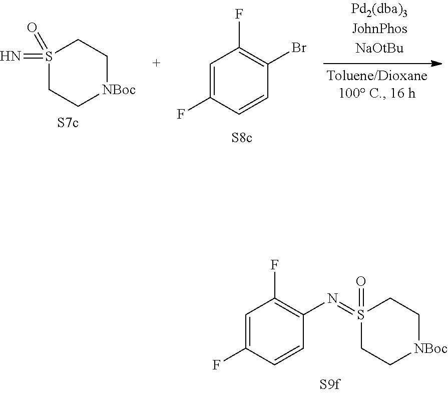

A microwave vial equipped with a stir bar was charged with tert-Butyl 1-iminothiomorpholine-4-carboxylate 1-oxide (S7c, 500 mg, 2.03 mmol), 1-bromo-2,4-difluorobenzene (S8c, 303 μL, 2.64 mmol), JohnPhos (90.7 mg, 304 μmol), sodium tert-butoxide (488 mg, 5.07 mmol) and tris(dibenzylideneacetone)-dipalladium(0) (97.7 mg, 101 μmol). The solids were diluted in a mixture of toluene (5.00 mL) and dioxane (5.00 mL) and then degassed by nitrogen gas bubbling (5 min). Then, the container was sealed and the mixture heated to 100° C. overnight. Afterwards, the reaction mixture was filtered through Celite and the solvents evaporated under reduced pressure. Reaction crude was purified by reverse phase column chromatography using ammonium formate/ACN as eluents to obtain S9f as off-white amorphous solid (400 mg, 51%).

### Step b. 4-(3-chloropyrido[3,4-b]pyrazin-2-yl)-1-((2,4-difluorophenyl)imino)-1l6-thiomorpholine 1-oxide (S10d)

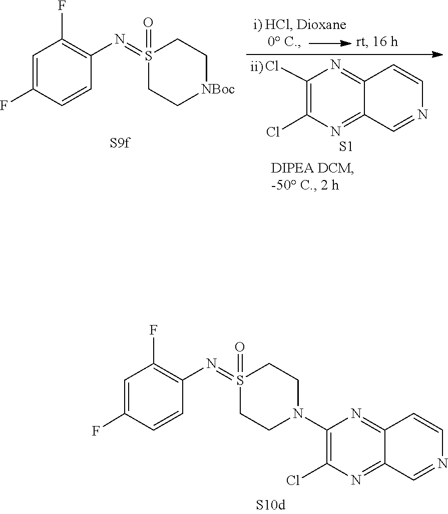

Compound S9f was deprotected following the procedure from Example 012—Step b. Compound S10d was synthesized from the deprotected s9f following the procedure described in Example 001—Step a and was obtained as a light reddish solid (43%).

### Step c. 4-(3-(1H-pyrazol-4-yl)pyrido[3,4-b]pyrazin-2-yl)-1-((2,4-difluorophenyl)imino)-1l6-thiomorpholine 1-oxide (Compound 58)

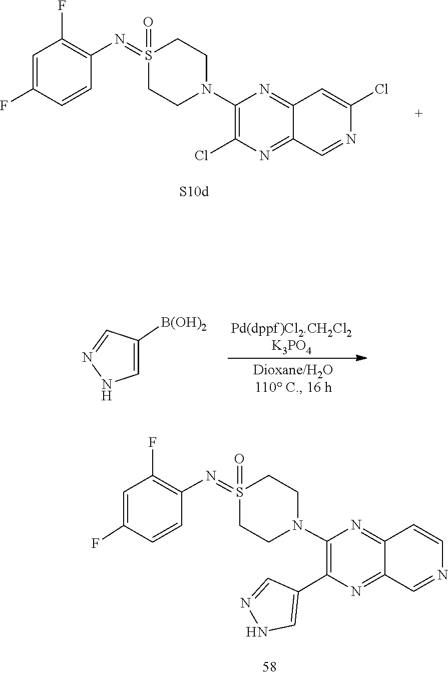 [SMILES: O=S1(=Nc2ccc(F)cc2F)CCN(c2nc3cc(Cl)ncc3nc2Cl)CC1 ; OB(O)c1cn[nH]c1 ; O=S1(=Nc2ccc(F)cc2F)CCN(c2nc3ccncc3nc2-c2cn[nH]c2)CC1]

Compound 58 was synthesized following the procedure described in Example 001—Step d using Pd(dppf)Cl₂·CH₂Cl₂ as catalyst and was obtained as a white powder (8.00 mg, 5.2%).

### Example 018. Scheme 3. 2-(4-(2,4-difluorobenzyl)-3-methylpiperazin-1-yl)-3-(1H-pyrazol-4-yl)pyrido[3,4-b]pyrazine (Compound 49)

### Step a. tert-butyl 4-(2,4-difluorobenzyl)-3-methylpiperazine-1-carboxylate (S15a)

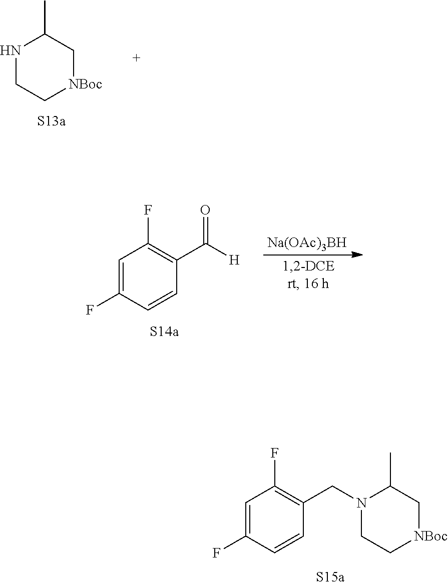 [SMILES: CC1CN(C(=O)OC(C)(C)C)CCN1 ; O=Cc1ccc(F)cc1F ; CC1CN(C(=O)OC(C)(C)C)CCN1Cc1ccc(F)cc1F ; "Na(OAc)3BH"]

In a 100 mL round bottom flask, to a solution of tert-Butyl 3-methylpiperazine-1-carboxylate (S13a, 345 mg, 1.67 mmol) and 2,4-difluorobenzaldehyde (S14a, 201 μL, 1.84 mmol) in 1,2-DCE (6.7 mL), sodium triacetoxyborohydride (746 mg, 3.35 mmol) was added. The reaction mixture was stirred at room temperature overnight. Afterwards, reaction was quenched by the addition of saturated sodium bicarbonate solution. Then, the reaction crude was transferred to a separatory funnel using DCM/water and extracted with DCM (3×). The organic phase was dried over sodium sulphate, filtered and concentrated. Compound 15 was obtained as a colorless oil (530 mg, 97%) and it was used in the next steps without additional purification.

### Step b. 3-chloro-2-(4-(2,4-difluorobenzyl)-3-methylpiperazin-1-yl)pyrido[3,4-b]pyrazine (S16a)

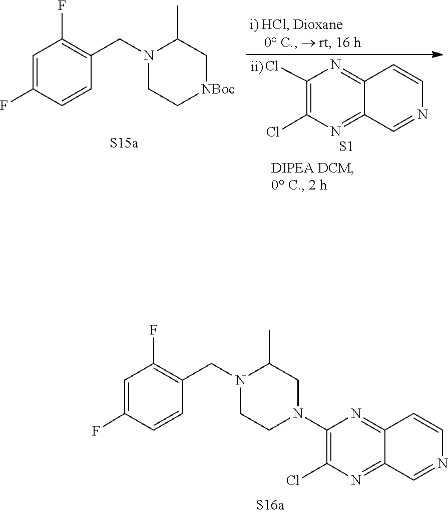

Compound S15a was deprotected following the procedure from Example 012—Step b. S16a was synthesized from the deprotected s15a following the procedure described in Example 001—Step a and was obtained as a yellow oil (30.0 mg, 4.8%).

### Step c. 2-(4-(2,4-difluorobenzyl)-3-methylpiperazin-1-yl)-3-(1H-pyrazol-4-yl)pyrido[3,4-b]pyrazine (Compound 49)

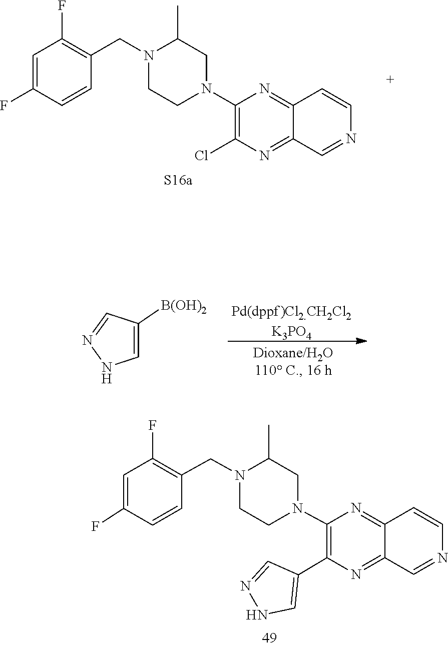 [SMILES: CC1CN(c2nc3ccncc3nc2Cl)CCN1Cc1ccc(F)cc1F ; OB(O)c1cn[nH]c1 ; CC1CN(c2nc3ccncc3nc2-c2cn[nH]c2)CCN1Cc1ccc(F)cc1F]

Compound 49 was synthesized following the procedure described in Example 001—Step d procedure using Pd(dppf)Cl₂·CH₂Cl₂ as catalyst and was obtained as a yellow powder (8.80 mg, 27%).

### Example 019. Scheme 3. 2-(4-(2,4-difluorobenzyl)-3-(methoxymethyl)piperazin-1-yl)-3-(1H-pyrazol-4-yl)pyrido[3,4-b]pyrazine (Compound 89)

### Step a. 1-(tert-butyl) 3-methyl 4-(2,4-difluorobenzyl)piperazine-1,3-dicarboxylate (S15b)

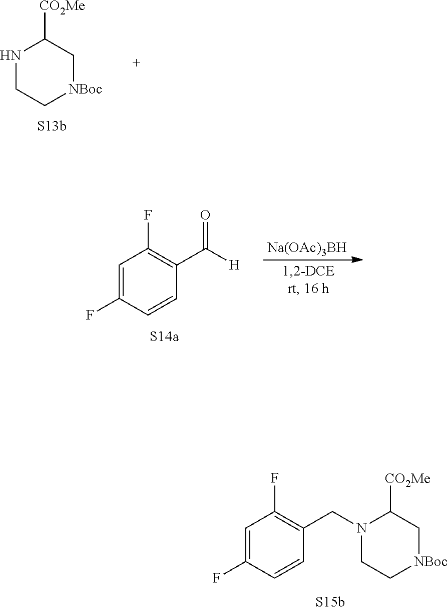 [SMILES: COC(=O)C1CN(C(=O)OC(C)(C)C)CCN1 ; O=Cc1ccc(F)cc1F ; COC(=O)C1CN(C(=O)OC(C)(C)C)CCN1Cc1ccc(F)cc1F ; "Na(OAc)3BH"]

Compound S15b was synthesized following reductive amination procedure described in Example 018—Step a and was obtained as a clear oil (2.51 g, 80%).

### tert-butyl 4-(2,4-difluorobenzyl)-3-(hydroxymethyl)piperazine-1-carboxylate (S15c)

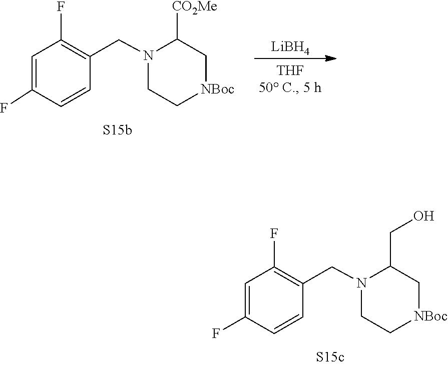 [SMILES: COC(=O)C1CN(C(=O)OC(C)(C)C)CCN1Cc1ccc(F)cc1F ; "LiBH4" ; CC(C)(C)OC(=O)N1CCN(Cc2ccc(F)cc2F)C(CO)C1]

In a 100 mL round bottom flask, S15b (2.50 g, 6.75 mmol) was dissolved in THF (30 mL). Then, solution of lithiumborohydride (4M in THF, 6.75 mL, 27.0 mmol) was added and the reaction mixture was stirred for 5 h at 50° C. Afterwards, reaction was quenched by the addition of saturated sodium bicarbonate solution and extracted with ethyl acetate (3×). The organic phase was dried over sodium sulphate, filtered and concentrated. Compound S15c was obtained as a green oil (930 mg, 40%) and it was used in the next steps without additional purification.

### tert-butyl 4-(2,4-difluorophenoxy)-4-(methoxymethyl)piperidine-1-carboxylate (S15d)

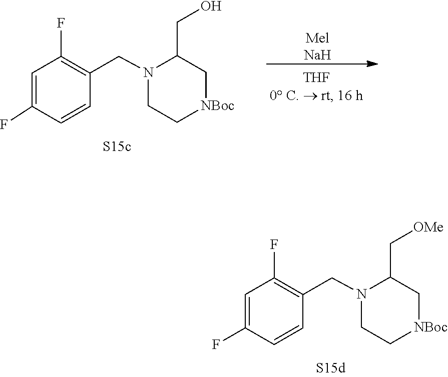 [SMILES: CC(C)(C)OC(=O)N1CCN(Cc2ccc(F)cc2F)C(CO)C1 ; "MelNaH" ; COCC1CN(C(=O)OC(C)(C)C)CCN1Cc1ccc(F)cc1F]

Compound S15d compound was synthesized following methylation procedure in Example 014—step a and was obtained as a colorless oil (100 mg, 48%).

### Step b. 3-chloro-2-(4-(2,4-difluorobenzyl)-3-(methoxymethyl)piperazin-1-yl)pyrido[3,4-b]pyrazine (S16b)

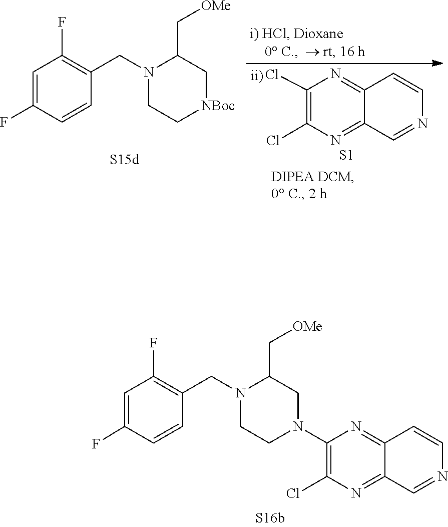

Compound S15d was deprotected following the procedure from Example 012—Step b. Compound S16b was synthesized from the deprotected S15d following the procedure described in Example 001—Step a and was obtained as an orange-brown semi-solid (115 mg, 88%).

### Step c. 2-(4-(2,4-difluorobenzyl)-3-(methoxymethyl)piperazin-1-yl)-3-(1H-pyrazol-4-yl)pyrido[3,4-b]pyrazine (Compound 89)

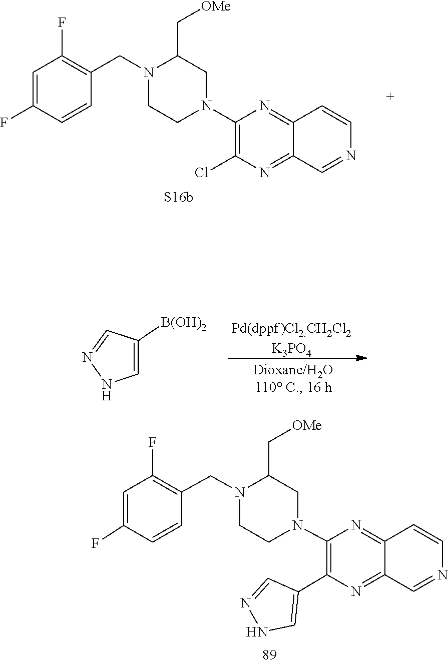 [SMILES: COCC1CN(c2nc3ccncc3nc2Cl)CCN1Cc1ccc(F)cc1F ; OB(O)c1cn[nH]c1 ; COCC1CN(c2nc3ccncc3nc2-c2cn[nH]c2)CCN1Cc1ccc(F)cc1F]

Compound 89 was synthesized following the procedure described in Example 001—Step d using Pd(dppf)Cl₂·CH₂Cl₂ as the catalyst and was obtained as an orange solid (12.5 mg, 10%).

### Example 020. Scheme 3. (4-(3-(1H-pyrazol-4-yl)pyrido[3,4-b]pyrazin-2-yl)-1-(2,4-difluorobenzyl)piperazin-2-yl)methanol (Compound 93)

### Step b. methyl 4-(3-chloropyrido[3,4-b]pyrazin-2-yl)-1-(2,4-difluorobenzyl)piperazine-2-carboxylate (S16c)

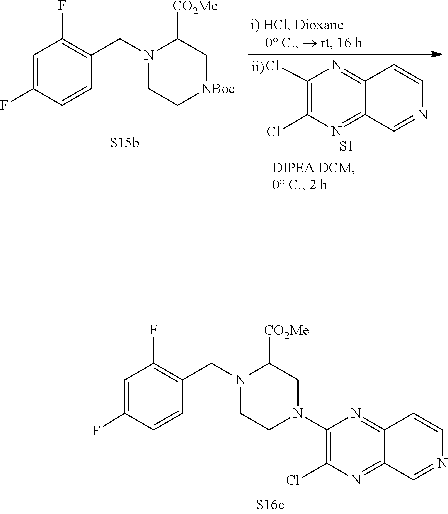

Compound S15b was deprotected following the procedure from Example 012—Step b. S16c was synthesized from the deprotected S15b following the procedure described in Example 001—Step a and was obtained as a yellow solid (400 mg, 73%).

### Step c. methyl 4-(3-(1H-pyrazol-4-yl)pyrido[3,4-b]pyrazin-2-yl)-1-(2,4-difluorobenzyl)piperazine-2-carboxylate (S17a)

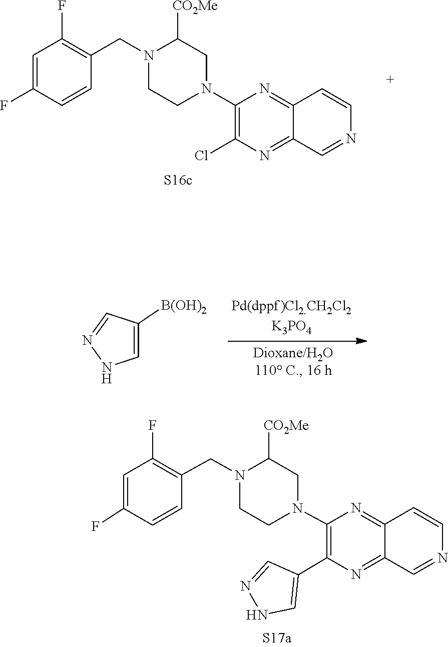 [SMILES: COC(=O)C1CN(c2nc3ccncc3nc2Cl)CCN1Cc1ccc(F)cc1F ; OB(O)c1cn[nH]c1 ; COC(=O)C1CN(c2nc3ccncc3nc2-c2cn[nH]c2)CCN1Cc1ccc(F)cc1F]

Compound S17a was synthesized following the procedure described in Example 001—Step d using Pd(dppf)Cl₂·CH₂Cl₂ as the catalyst and was obtained as a brown solid (50.0 mg, 47%).

### Step e. (4-(3-(1H-pyrazol-4-yl)pyrido[3,4-b]pyrazin-2-yl)-1-(2,4-difluorobenzyl)piperazin-2-yl)methanol (Compound 93)

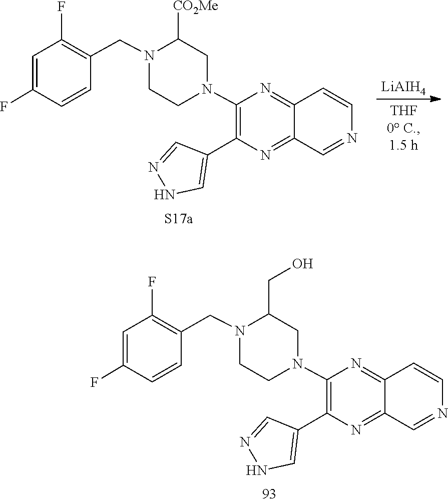

In a 10 mL round bottom flask under nitrogen atmosphere, S17a (15.0 mg, 32.2 μmol) was dissolved in anhydrous THF (0.75 mL) and the mixture cooled down to 0° C. Then, solution of lithium aluminum hydride (1 M in THF, 64.5 μL, 64.5 μmol) was added and the reaction mixture was stirred for 1.5 h at 0° C. Afterwards, reaction was quenched by the addition of drops of saturated ammonium chloride solution. The solvents were evaporated and the crude directly purified by reverse phase column chromatography using ammonium formate/ACN as eluents to obtain Compound 93 as a beige solid (6.49 mg, 46%).

### Example 021. Scheme 4. 1-(2-(4-(2,4-difluorobenzyl)piperazin-1-yl)-3-(6-methoxypyrazin-2-yl)pyrido[3,4-b]pyrazin-7-yl)-2-methylpropane-1,2-diol (Compound 94)

### Step a. 3,7-dichloro-2-(4-(2,4-difluorobenzyl)piperazin-1-yl)pyrido[3,4-b]pyrazine (S21a)

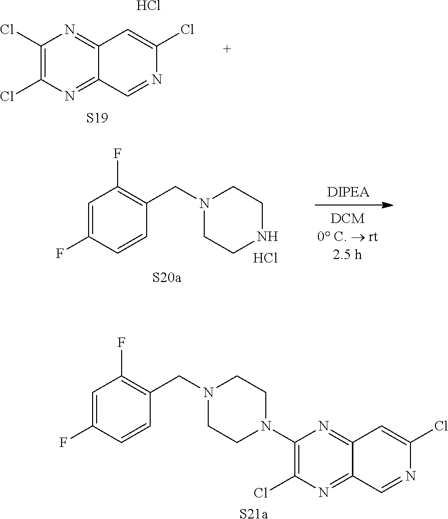

NOTE: compound S19 was prepared following the report in WO2019/231270, 2019, A1.

A round-bottom flask equipped with a stir bar was charged with 2,3,7-trichloropyrido[3,4-b]pyrazine hydrochloride (S19, 2.50 g, 9.23 mmol), 1-(2,4-difluorobenzyl)piperazine hydrochloride (S20a, 2.41 g, 9.69 mmol) and DCM (92.3 mL). The resulting mixture was cool down to 0° C. where N,N-diisopropylethylamine (7.06 mL, 40.5 mmol) was added dropwise. The mixture was allowed to warm up to room temperature and stirred for 2.5 h. Afterwards, the reaction mixture was diluted with cold ethyl acetate (100 mL) and partitioned with cold water. The system was extracted with ethyl acetate (3×). The organic layer was washed with brine and dried over sodium sulfate. Following filtration and evaporation of volatiles, S21a was generated as a brown solid (3.73 g, 99%).

### Step c. 7-chloro-2-(4-(2,4-difluorobenzyl)piperazin-1-yl)-3-(6-methoxypyrazin-2-yl)pyrido[3,4-b]pyrazine (S22a)

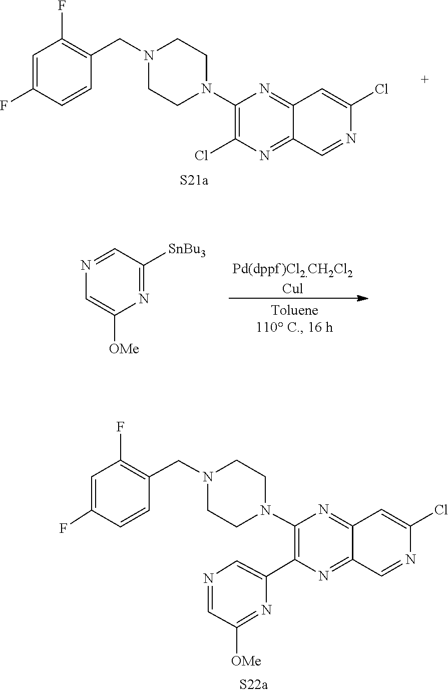

Compound S22a was synthesized following the procedure described in Example 003—Step e using Pd(dppf)Cl₂·CH₂Cl₂ as the catalyst and was obtained as an orange solid (197 mg, 48%).

### Step d. 2-(4-(2,4-difluorobenzyl)piperazin-1-yl)-3-(6-methoxypyrazin-2-yl)-7-(2-methylprop-1-en-1-yl)pyrido[3,4-b]pyrazine (S24a)

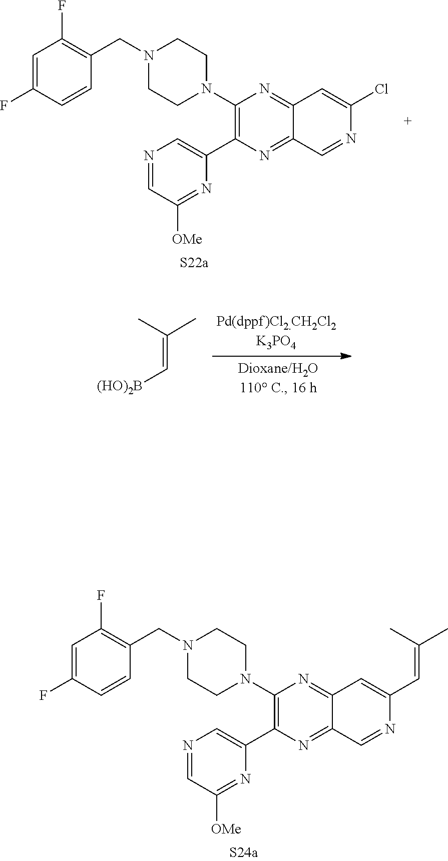

Compound S24a was synthesized following the procedure described in Example 001—Step d using Pd(dppf)Cl₂·CH₂Cl₂ as the catalyst and was obtained as an orange solid (114 mg, 56%).

### Step e. 1-(2-(4-(2,4-difluorobenzyl)piperazin-1-yl)-3-(6-methoxypyrazin-2-yl)pyrido[3,4-b]pyrazin-7-yl)-2-methylpropane-1,2-diol (Compound 94)

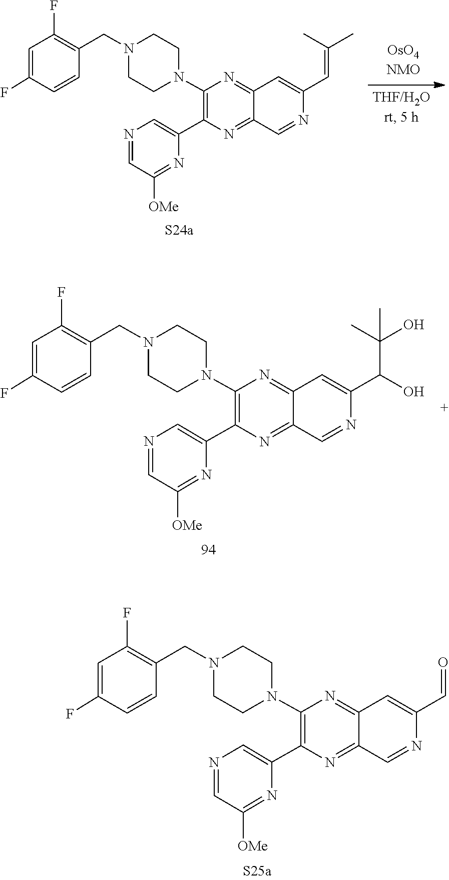 [SMILES: COc1cncc(-c2nc3cnc(C=C(C)C)cc3nc2N2CCN(Cc3ccc(F)cc3F)CC2)n1 ; COc1cncc(-c2nc3cnc(C(O)C(C)(C)O)cc3nc2N2CCN(Cc3ccc(F)cc3F)CC2)n1 ; COc1cncc(-c2nc3cnc(C=O)cc3nc2N2CCN(Cc3ccc(F)cc3F)CC2)n1]

In a 10 mL round bottom flask, S24a (110 mg, 218 μmol) and 4-methylmorpholine N-oxide (NMO, 52.8 mg, 437 μmol) were dissolved in a mixture of THF (1.82 mL) and H2O (364 μL). Then, the mixture was treated with 4% in water solution of osmium tetroxide (278 μL, 43.7 μmol). The reaction mixture was stirred at room temperature for 5 h (reaction completion indicated by LCMS analysis). Afterwards, the reaction mixture was filtered through a silica/celite system and the filtrate concentrated to be directly purified by reverse phase column chromatography using ammonium formate/ACN as eluents to obtain Compound 94 as a yellow powder (17.0 mg, 14%), and S25a as an orange solid (16.2 mg, 16%).

### Example 022. Scheme 4. (2-(4-(2,4-difluorobenzyl)piperazin-1-yl)-3-(6-methoxypyrazin-2-yl)pyrido[3,4-b]pyrazin-7-yl)methanol (Compound 95)

### Step f. 1-(2-(4-(2,4-difluorobenzyl)piperazin-1-yl)-3-(6-methoxypyrazin-2-yl)pyrido[3,4-b]pyrazin-7-yl)-2-methylpropane-1,2-diol (Compound 95)

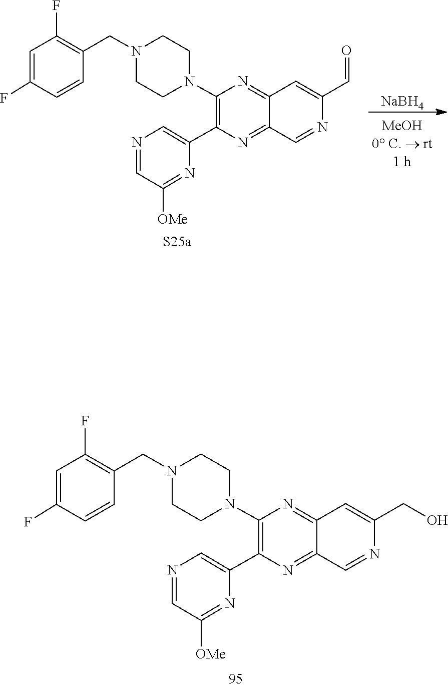

In a 10 mL round bottom flask, S25a (16.0 mg, 33.5 μmol) was dissolved in MeOH (1 mL) and the mixture cooled down to 0° C. Then, sodium borohydride (1.94 mg, 50.3 μmol) was added slowly and the system stirred for 1 hour at room temperature (reaction completion indicated by LCMS analysis). Afterwards, the solvents were evaporated and the obtained crude directly purified by reverse phase column chromatography using ammonium bicarbonate/ACN as eluents to obtain Compound 95 as a yellow powder (8.00 mg, 50%).

### Example 023. Scheme 5. 3-(4-(2,5-dichlorobenzyl)piperazin-1-yl)-2-(1H-imidazol-2-yl)pyrido[3,4-b]pyrazine (Compound 42)

### Step a. 3-chloro-2-((4-methoxybenzyl)oxy)pyrido[3,4-b]pyrazine (40-9b)

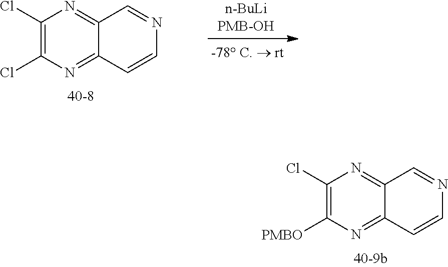

To the solution of PMB-OH (1 g, 7.2 mmol, 1.0 eq) in THF (10 mL) at −78° C. was added n-BuLi (2.5 M in hexane) (3.4 mL, 8.6 mmol, 1.2 eq) under nitrogen atmosphere. After 30 min at same temperature added solution of 40-8 (1.4 g, 7.2 mmol, 1 eq, dissolved in 10 mL of THF). The reaction mass temperature was allowed to 25-30° C. and stirred for 1-2h and was monitored by TLC. After completion, the reaction was quenched with a saturated NH₄Cl solution (100 mL) at −20° C. and extracted with EtOAc (2×100 mL). Finally, the organic layer was dried over anhydrous Na₂SO₄, filtered and evaporated under reduced pressure to obtain crude 40-9b (1.8 g, quantitative yield) as brown liquid which contains ˜35% of desired product by LCMS. The crude was used in next step without purification.

### Step b. 3-(4-(5-chloro-2-fluorobenzyl)piperazin-1-yl)-2-((4-methoxybenzyl)oxy)pyrido[3,4-b]pyrazine (40-14a)

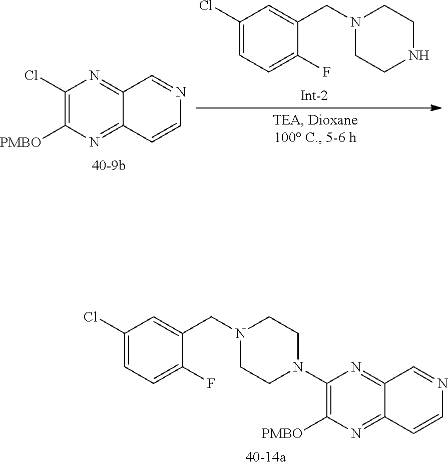

To the solution of 40-9b (1 g, 3.3 mmol, 1.0 eq) and Int-2 (700 mg, 2.9 mmol, 0.9 eq) in 1,4-Dioxane (10 mL) at room temperature was added TEA (0.7 mL, 9.9 mmol, 3.0 eq). The reaction mixture temperature was raised to 100° C. and stirred for 5 to 6 h. The progress of the reaction was monitored by TLC. After completion, it was cooled to rt then diluted with EtOAc (50 mL) and washed with water (2×50 mL). Finally, the organic layer was dried over anhydrous Na₂SO₄ filtered and evaporated under reduced pressure to obtain crude (1 g) as brown solid. The crude product was purified by combi flash reverse phase column (30 g) eluted with 60-80% Acetonitrile in water. Combined product fractions were extracted with DCM (200 mL×1), the DCM layer was dried over anhydrous Na₂SO₄, filtered and concentrated under reduced pressure to afford 40-14a (900 mg, 55%) as brown liquid.

### Step c. 3-(4-(5-chloro-2-fluorobenzyl)piperazin-1-yl)pyrido[3,4-b]pyrazin-2-ol (40-15a)

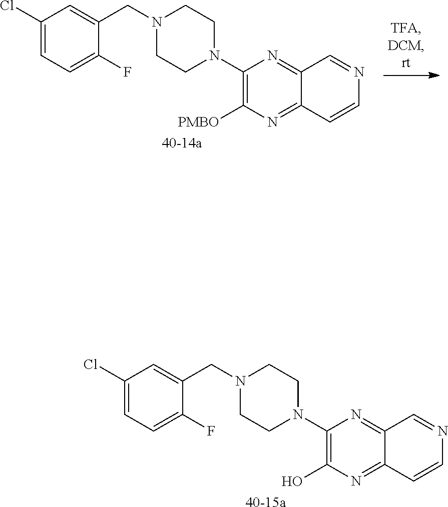

To the 40-14a (830 mg) in DCM (10 mL) at 0° C. was added TFA (10 mL). The reaction mixture was stirred at 25-30° C. for 2 h. The progress of the reaction was monitored by TLC. After completion, it was diluted with DCM (50 mL) and washed with sat. NaHCO₃ solution (25 mL×2). Finally, the organic layer was dried over anhydrous Na₂SO₄, filtered and evaporated to get crude (150 mg) as brown gum. The crude product was purified by silica gel column (60-120) eluted with 0-5% MeOH in DCM. Concentrated the product fractions to afford the title 40-15a (500 mg, 79%) as yellow gum.

### Step d. 2-chloro-3-(4-(5-chloro-2-fluorobenzyl)piperazin-1-yl)pyrido[3,4-b]pyrazine (40-16a)

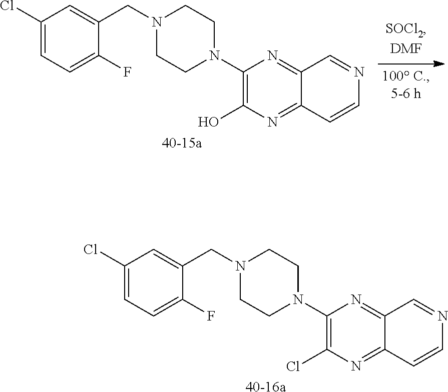 [SMILES: Oc1nc2ccncc2nc1N1CCN(Cc2cc(Cl)ccc2F)CC1 ; "40-15a" ; Fc1ccc(Cl)cc1CN1CCN(c2nc3cnccc3nc2Cl)CC1 ; "40-16a"]

To the neat 40-15a (500 mg) was added SOCl₂ (5 ML) and DMF (0.2 mL). The reaction mixture was heated to 100° C. for 5-6h. The progress of the reaction was monitored by TLC. After completion of the reaction evaporated the reaction mass to get brown residue. The residue was dissolved in EtOAc (50 mL) and washed with cold sat. NaHCO₃ solution (20 mL×2). Finally, the organic layer was dried over anhydrous Na₂SO₄, filtered and evaporated to get crude (200 mg) as brown solid. The crude product was purified by silica gel column (60-120) eluted with 15-20% EtOAc in Hexane and concentrated to afford 40-16a (80 mg, 15%) as yellow liquid.

### Step e. 3-(4-(5-chloro-2-fluorobenzyl)piperazin-1-yl)-2-(1-(methoxymethyl)-1H-imidazol-2-yl)pyrido[3,4-b]pyrazine (40-17a)

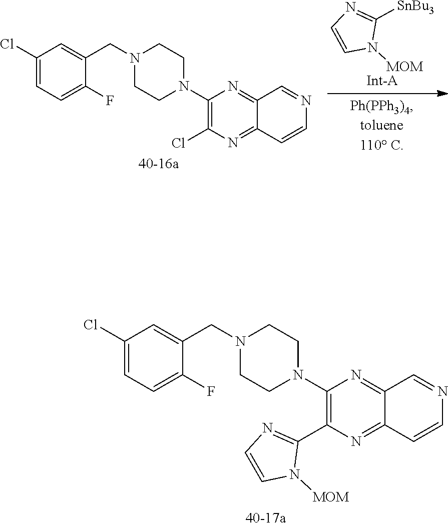

To the solution of 40-16a (80 mg, 0.20 mmol, 1 eq) and Int-A (123 mg, 0.30 mmol, 1.5 eq) in Toluene (10 mL) at rt was degassed with nitrogen for 10-15 min then added Pd(PPh₃)₄ (23 mg, 0.02 mmol, 0.1 eq). The reaction mixture temperature was raised to 100-110° C. and stirred for 16 h. The progress of the reaction was monitored by TLC. After completion, it was evaporated to get crude compound (150 mg) as brown gum. The crude product was purified by silica gel column (60-120) eluted with 5-10% MeOH in DCM and concentrated to afford 40-17a (60 mg, 63%) as yellow solid.

### Step f. 3-(4-(2,5-dichlorobenzyl)piperazin-1-yl)-2-(1H-imidazol-2-yl)pyrido[3,4-b]pyrazine (Compound 42)

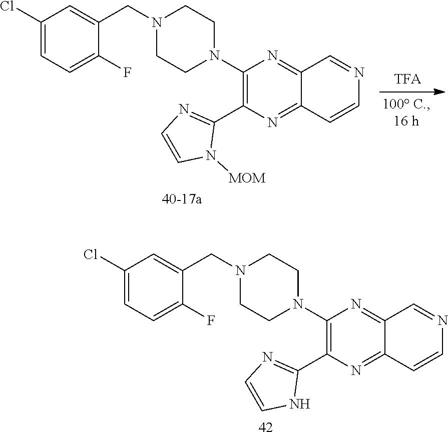

To 40-13a (60 mg) was added TFA (5 mL). The reaction mixture was heated to 90° C.-100° C. for 16 h. The progress of the reaction was monitored by TLC. After completion, it was evaporated to get crude compound as brown gum which was dissolved in DCM (50 mL) and washed with sat. NaHCO₃ solution (20 mL×2). Finally, the organic layer was dried over anhydrous Na₂SO₄, filtered and evaporated to get crude (50 mg) as brown gum. The crude product was purified by reverse phase column (6 g) and eluted with 40-60% of acetonitrile in water as mobile phase. The product fractions were lyophilized to afford Compound 42 (6 mg, 11%) as pale-yellow solid.

### Example 24. Scheme 4. 1-[7-chloro-3-(1-methyl-1H-pyrazol-4-yl)pyrido[3,4-b]pyrazin-2-yl]-4-[(2,4-difluorophenyl)methyl]piperazine (Compound 193) and 2-{4-[(2,4-difluorophenyl)methyl]piperazin-1-yl}-3-(1-methyl-1H-pyrazol-4-yl)pyrido[3,4-b]pyrazine-7-carbonitrile (Compound 216)

### Step b. 1-[7-chloro-3-(1-methyl-1H-pyrazol-4-yl)pyrido[3,4-b]pyrazin-2-yl]-4-[(2,4-difluorophenyl)methyl]piperazine (Compound 193)

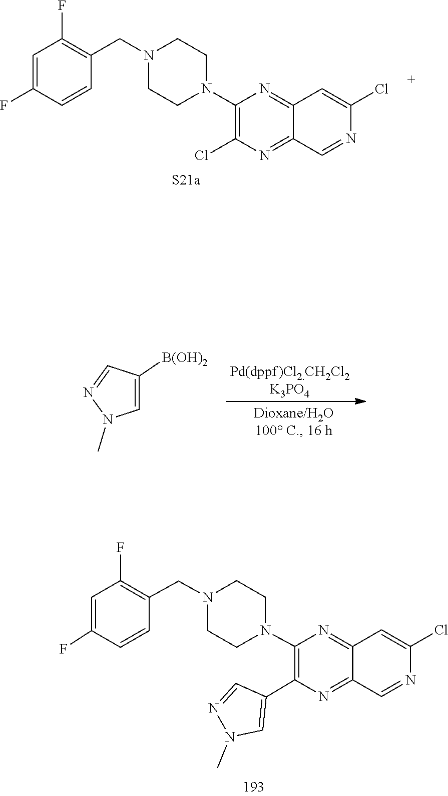

Compound 193 was synthesized following the procedure described in Example 001—Step d using Pd(dppf)Cl₂·CH₂Cl₂ as catalyst and was obtained as a light yellow power (38%).

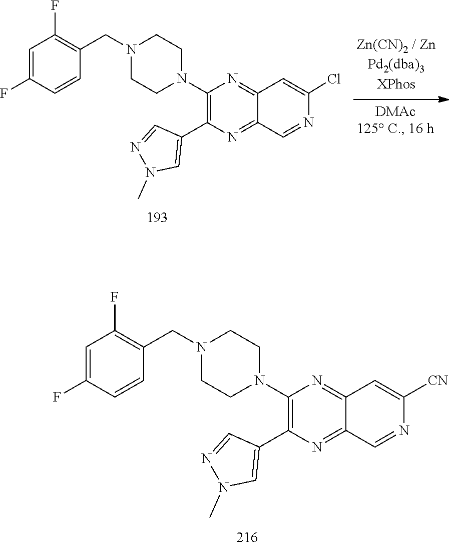 [SMILES: Cn1cc(-c2nc3cnc(Cl)cc3nc2N2CCN(Cc3ccc(F)cc3F)CC2)cn1 ; Cn1cc(-c2nc3cnc(C#N)cc3nc2N2CCN(Cc3ccc(F)cc3F)CC2)cn1]

In a microwave vial, Compound 193 (50.0 mg, 110 umol), Zinc dust (1.44 mg, 21.9 umol), Zinc cyanide (15.8 mg, 132 umol), 2-dicyclohexylphosphino-2′,4′,6′-triisopropylbiphenyl (XPhos, 10.8 mg, 21.9 umol) and Tris(dibenzylideneacetone)-dipalladium(0) (10.0 mg, 11.0 umol) were dissolved in DMAc (1.08 mL). The obtained mixture was degassed for 15 minutes using nitrogen gas bubbling, then, the system was sealed and heated at 125° C. overnight. Afterwards, the mixture was diluted with EtOAc, filtered through celite/silica layer, and the filtrate concentrated under reduced pressure to be directly purified by reverse phase column chromatography using ammonium formate/ACN as eluents to obtain Compound 216 as yellow powder (15.0 mg, 31%).

### Example 25. Scheme 4. 2-{4-[(2,4-difluorophenyl)methyl]piperazin-1-yl}-3-(1-methyl-1H-pyrazol-4-yl)pyrido[3,4-b]pyrazin-7-amine (Compound 226)

### Step g. 2-{4-[(2,4-difluorophenyl)methyl]piperazin-1-yl}-3-(1-methyl-1H-pyrazol-4-yl)pyrido[3,4-b]pyrazin-7-amine (Compound 226)

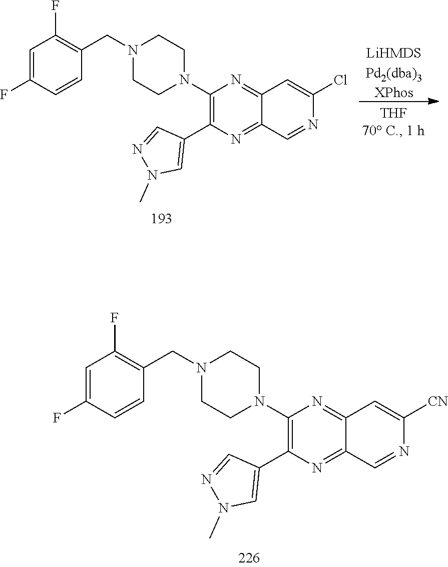 [SMILES: Cn1cc(-c2nc3cnc(Cl)cc3nc2N2CCN(Cc3ccc(F)cc3F)CC2)cn1 ; "LiHMDSPd2(dba)3XPhos" ; Cn1cc(-c2nc3cnc(C#N)cc3nc2N2CCN(Cc3ccc(F)cc3F)CC2)cn1]

In a microwave vial, Compound 193 (100 mg, 219 umol), 2-dicyclohexylphosphino-2′,4′,6′-triisopropylbiphenyl (XPhos, 43.1 mg, 87.7 umol) and Tris(dibenzylideneacetone)-dipalladium(0) (40.2 mg, 43.9 umol) were dissolved in anhydrous THF (2.19 mL), then, solution of lithium bis(trimethylsilyl)amide (1 M in THF, 439 uL, 439 umol) was added. The obtained mixture was degassed for 15 minutes using nitrogen gas bubbling, then, the system was sealed and heated at 70° C. for 1 h. Afterwards, the mixture was diluted with EtOAc, filtered through celite/silica layer, and the filtrate concentrated under reduced pressure to be directly purified by reverse phase column chromatography using ammonium formate/ACN as eluents to obtain Compound 226 as orange fluorescent powder (24.0 mg, 25%).

### Example 26. Scheme 4. 7-chloro-2-(4-(2,4-difluorophenoxy)piperidin-1-yl)-3-(1-methyl-1H-pyrazol-4-yl)pyrido[3,4-b]pyrazine (Compound 289), 1-(2-(4-(2,4-difluorophenoxy)piperidin-1-yl)-3-(1-methyl-1H-pyrazol-4-yl)pyrido[3,4-b]pyrazin-7-yl)-2-methylpropane-1,2-diol (Compound 263), and cyclopropyl(2-(4-(2,4-difluorophenoxy)piperidin-1-yl)-3-(1-methyl-1H-pyrazol-4-yl)pyrido[3,4-b]pyrazin-7-yl)methanol (Compound 250)

### Step a. 3,7-dichloro-2-(4-(2,4-difluorophenoxy)piperidin-1-yl)pyrido[3,4-b]pyrazine (S21a)

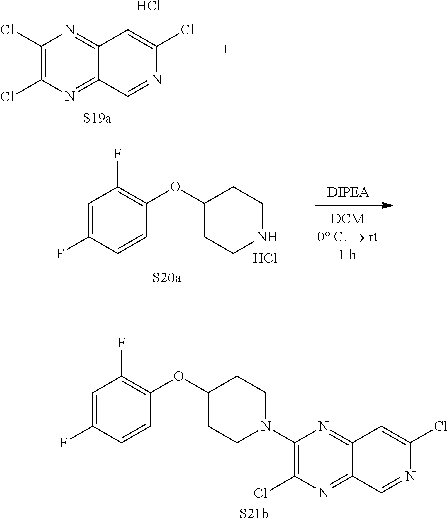

The compound S21b was synthesized following Example 001—Step d. and was obtained as yellow solid (7.0 g, 90%).

### Step b. 7-chloro-2-(4-(2,4-difluorophenoxy)piperidin-1-yl)-3-(1-methyl-1H-pyrazol-4-yl)pyrido[3,4-b]pyrazine (Compound 289)

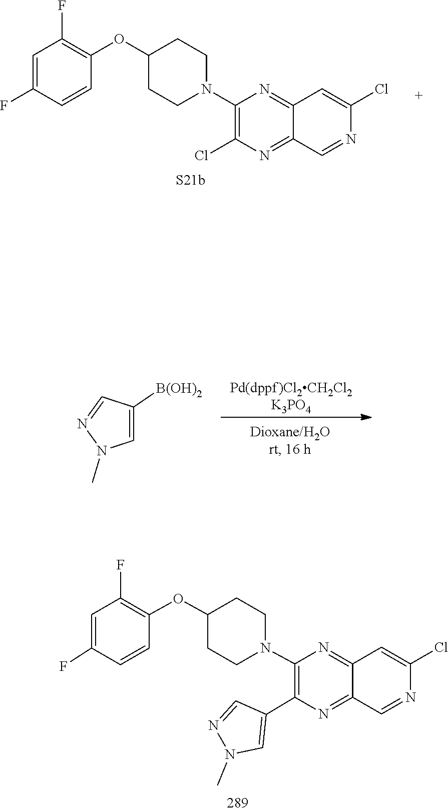

The compound 289 was synthesized following Example 001—Step d procedure at room temperature using Pd(dppf)Cl₂·CH₂Cl₂ as catalyst and was obtained as yellow powder (1.95 g, 59%).

### Step d. 2-(4-(2,4-difluorophenoxy)piperidin-1-yl)-3-(1-methyl-1H-pyrazol-4-yl)-7-(2-methylprop-1-en-1-yl)pyrido[3,4-b]pyrazine (S24b)

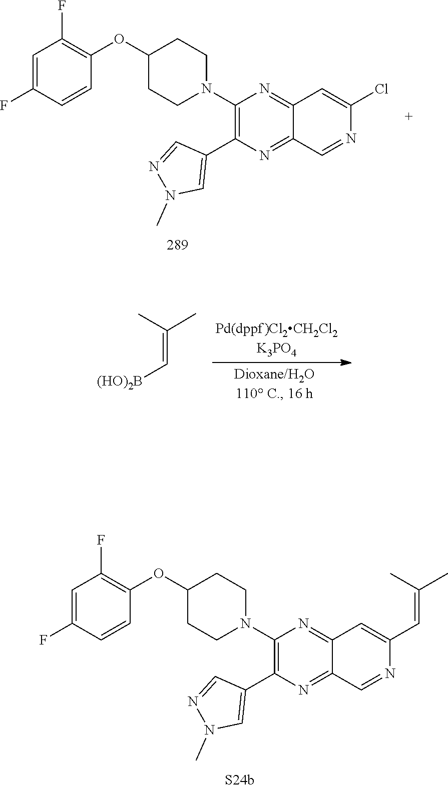

The compound S24b was synthesized following Example 001—Step d procedure using Pd(dppf)Cl₂·CH₂Cl₂ as catalyst. S24b was obtained as orange solid (278 mg, 89%).

### Step e. 1-(2-(4-(2,4-difluorophenoxy)piperidin-1-yl)-3-(1-methyl-1H-pyrazol-4-yl)pyrido[3,4-b]pyrazin-7-yl)-2-methylpropane-1,2-diol (Compound 263)

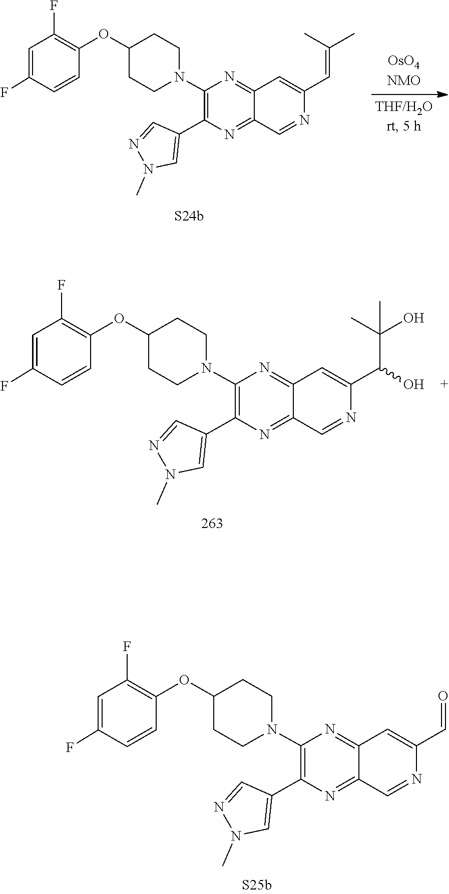 [SMILES: CC(C)=Cc1cc2nc(N3CCC(Oc4ccc(F)cc4F)CC3)c(-c3cnn(C)c3)nc2cn1 ; Cn1cc(-c2nc3cnc(C(O)C(C)(C)O)cc3nc2N2CCC(Oc3ccc(F)cc3F)CC2)cn1 ; Cn1cc(-c2nc3cnc(C=O)cc3nc2N2CCC(Oc3ccc(F)cc3F)CC2)cn1]

Compound 263 was synthesized following Example 021—Step e. and was obtained as beige solid (33 mg, 13%). Aldehyde S25b was obtained as brown solid (30 mg, 14%).

### Step f. cyclopropyl(2-(4-(2,4-difluorophenoxy)piperidin-1-yl)-3-(1-methyl-1H-pyrazol-4-yl)pyrido[3,4-b]pyrazin-7-yl)methanol (Compound 250)

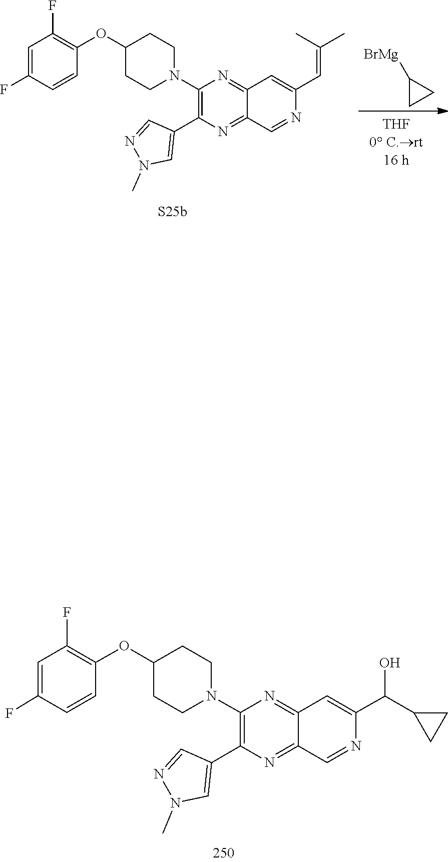

In a reaction vial, S25b (30.0 mg, 0.07 mmol) was dissolved in anhydrous THF (2 mL) and the mixture cooled down to 0° C. Then, cyclopropylmagnesium bromide solution (0.5 M in THF, 160 uL, 0.08 mmol) was added slowly and the system stirred overnight at room temperature (reaction completion indicated by LCMS analysis). Afterwards, reaction was quenched by the addition of ammonium chloride solution. The system was extracted with Ethyl Acetate (3×). The organic layer was washed with brine and dried over sodium sulfate. Followed by filtration and evaporation of volatiles, the obtained crude was purified by reverse phase column chromatography using ammonium formate/ACN as eluents to obtain Compound 250 as yellow powder (4.77 mg, 15%).

### Example 27. 2-amino-1-(2-(4-(2,4-difluorophenoxy)piperidin-1-yl)-3-(1-methyl-1H-pyrazol-4-yl)pyrido[3,4-b]pyrazin-7-yl)ethan-1-ol

### Step a. 2-(4-(2,4-difluorophenoxy)piperidin-1-yl)-3-(1-methyl-1H-pyrazol-4-yl)-7-vinylpyrido[3,4-b]pyrazine (S24c)

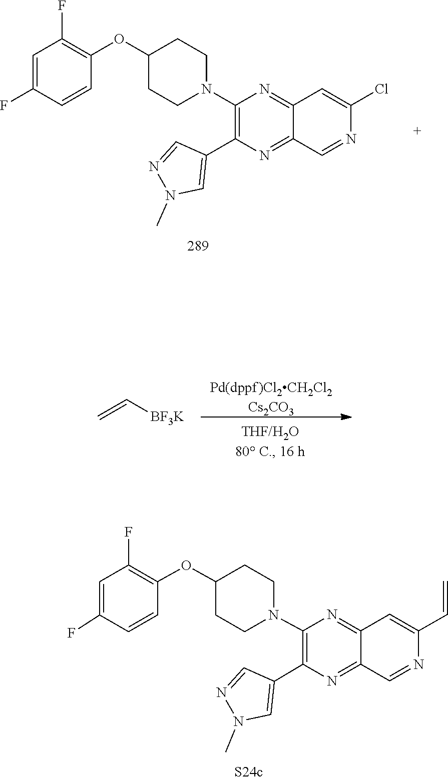

In a reaction vial, a mixture of Compound 289 (240 mg, 525 umol), potassium vinyltrifluoroborate (215 mg, 1.58 mmol), bis(diphenylphosphino)ferrocene]dichloropalladium(ll), complex with dichloromethane (87.5 mg, 105 umol) and cesium carbonate (513 mg, 1.58 mmol) was dissolved in a mixture of THF (2.50 mL) and H₂O (250 uL). The system was degassed for 5 min using a flow of nitrogen and then heated at 80° C. overnight. The mixture was filtered through a layer of celite/silica using EtOAc. Then, the solvents were evaporated, and the crude was directly purified by column chromatography over silica gel using Hexanes/EtOAc as eluents, affording the title compound S24c (186 mg, 76%) as bright yellow oil.

### Step f. 2-(4-(2,4-difluorophenoxy)piperidin-1-yl)-3-(1-methyl-1H-pyrazol-4-yl)-7-(oxiran-2-yl)pyrido[3,4-b]pyrazine (S26a)

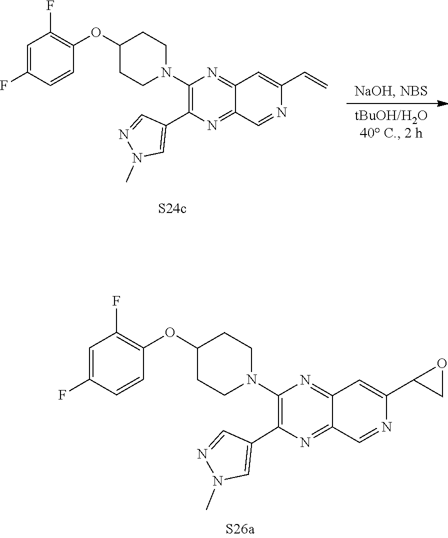 [SMILES: C=Cc1cc2nc(N3CCC(Oc4ccc(F)cc4F)CC3)c(-c3cnn(C)c3)nc2cn1 ; Cn1cc(-c2nc3cnc(C4CO4)cc3nc2N2CCC(Oc3ccc(F)cc3F)CC2)cn1]

In a 50 mL round-bottom flask equipped with a stir bar, S24c (180 mg, 401 umol) was dissolved in a mixture of t-Butanol (9.00 mL) and H₂O (18.0 mL). Then, N-bromosuccinimide (NBS, 79.4 mg, 442 umol) was added slowly. The reaction mixture was heated to 40° C. and stirred for 2 h where LCMS analysis indicated formation of the bromohydrin intermediate. Afterwards, the system was cooled to 0° C. and NaOH (1 M in H₂O. 134 μL, 134 umol) was added. The reaction mixture was let warmed and stirred at room temperature for 1 h. Then, reaction was quenched with the addition of water (50 mL) and extracted with DCM (3×). The organic layer was washed with brine, dried over anhydrous MgSO₄, filtered, and concentrated under reduced pressure to give the crude residue. The crude was purified by column chromatography over silica gel using DCM/MeOH as eluents, affording the title compound S26a (186 mg, 76%) as yellow solid.

### Step f. 2-amino-1-(2-(4-(2,4-difluorophenoxy)piperidin-1-yl)-3-(1-methyl-1H-pyrazol-4-yl)pyrido[3,4-b]pyrazin-7-yl)ethan-1-ol (Compound 309)

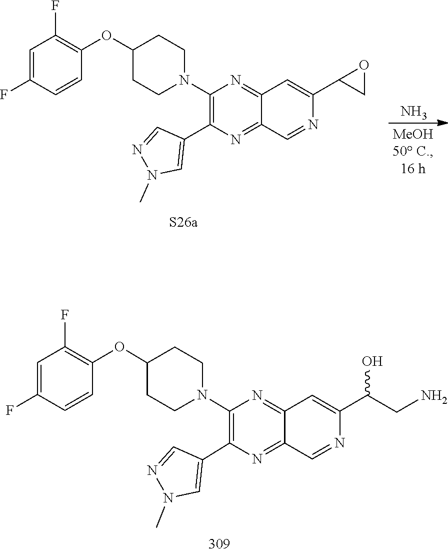

In a 25 mL round bottom flask, compound S26A (68.0 mg, 146 umol) was dissolved in ammonia solution (7 M in MeOH, 2.09 mL, 14.6 mmol). Then, the system was heated at 50° C. overnight. The reaction mixture was concentrated under vacuum and the crude was directly purified by reverse phase column chromatography using ammonium formate/ACN as eluents, affording Compound 309 (32.0 mg, 45%) as a yellow solid.

### Example 28. Scheme 5. 1-[7-chloro-2-(1-methyl-1H-pyrazol-4-yl)pyrido[3,4-b]pyrazin-3-yl]-4-[(2,4-difluorophenyl)methyl]piperazine (Compound 193) and 1-(3-(4-(2,4-difluorobenzyl)piperazin-1-yl)-2-(1-methyl-1H-pyrazol-4-yl)pyrido[3,4-b]pyrazin-7-yl)-2-methylpropane-1,2-diol (Compound 197)

### Step a. 3,7-dichloro-2-((4-methoxybenzyl)oxy)pyrido[3,4-b]pyrazine (S29a)

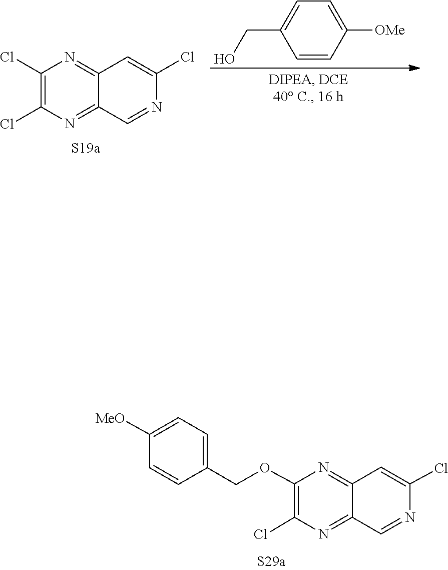

In a 250 mL round-bottom flask, to a solution of 4-methoxybenzyl alcohol (1.56 g, 11.1 mmol) and 2,3,7-trichloropyrido[3,4-b]pyrazine (S19a, 3.00 g, 11.1 mmol) in DCE (36.9 mL), N,N-Diisopropylethylamine (19.5 mL, 111 mmol) was added and the reaction mixture was stirred at 40° C. overnight. Afterwards, DCM and water were added and the reaction mixture was transferred to an extraction funnel. The organic layer was washed with brine and dried over sodium sulphate, filtered and concentrated under vacuum. The crude product was purified by column chromatography over silica gel using DCM/MeOH as eluents, affording compound S29a (1.40 g, 38%) as beige solid.

### Step b. 7-chloro-3-(4-(2,4-difluorobenzyl)piperazin-1-yl)-2-((4-methoxybenzyl)oxy)pyrido[3,4-b]pyrazine (S30a)

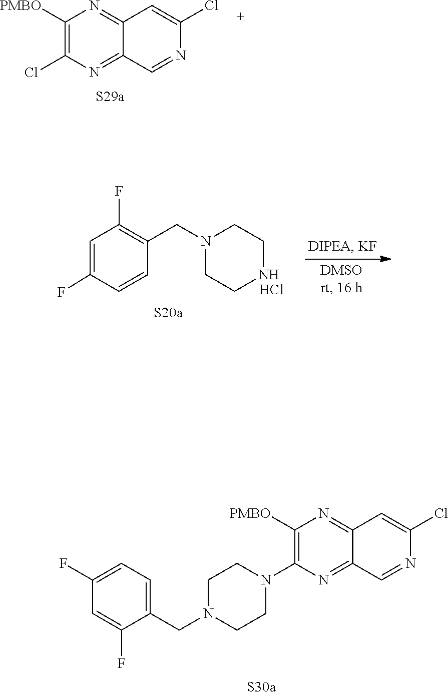

In a 100 mL round-bottom flask, compound S29a (1.40 g, 4.16 mmol), 1-[(2,4-difluorophenyl)methyl]piperazine dihydrochloride (S20a, 1.44 g, 4.79 mmol) and potassium fluoride (244 mg, 4.16 mmol) were dissolved in DMSO (20.8 mL). Then, N,N-Diisopropylethylamine (2.92 mL, 16.7 mmol) was added slowly and the mixture was stirred at room temperature overnight. Formation of precipitate was observed during the curse of the reaction. Water was added to the mixture and the solid was filtered (Note: additional product could be recovered from filtrate). The solid cake was washed with water and ether affording the title compound S30a (2.13 g, quantitative yield) as beige solid.

### Step c. 7-chloro-3-(4-(2,4-difluorobenzyl)piperazin-1-yl)pyrido[3,4-b]pyrazin-2-ol (S31b)

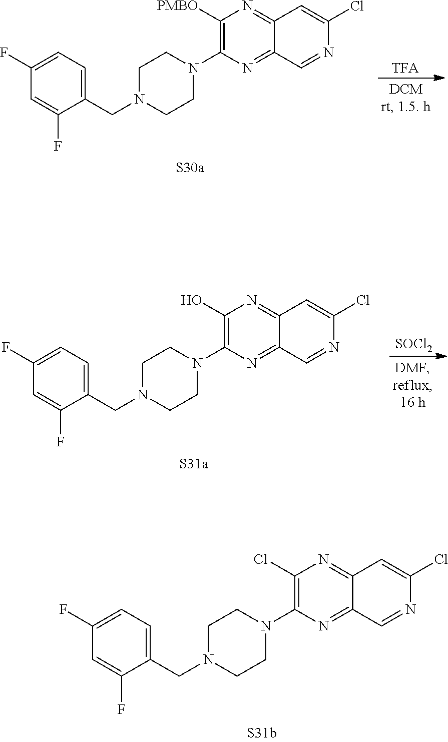

Step 1. In a 50 mL round-bottom flask equipped with a stir bar, trifluoroacetic acid (6.00 mL, 77.5 mmol) was added to a solution of S30a (572 mg, 1.12 mmol) in DCM (6.00 mL). The reaction was stirred at room temperature for 1.5 h (complete conversion indicated by LCMS). Afterwards, The reaction mixture was concentrated and the crude product was purified by column chromatography over silica gel using DCM/MeOH as eluents, affording compound S31a (438 mg, 100%) as an off-white solid.

Step 2. In a 25 mL round-bottom flask equipped with a stir bar, compound S31a (1.00 g, 2.55 mmol) was dissolved in thionyl chloride (7.09 mL). Then, DMF (198 uL, 2.55 mmol) was added dropwise and the reaction was heated to reflux (80° C.) and stirred overnight. Afterwards, the reaction was cooled to room temperature, SOCl₂ was evaporated under vacuum and then ice was added to the crude. The obtained solid was filtered and washed with cold water, cold MeCN and Et₂O, affording compound S31b (744 mg, 71%) as yellow solid.

### Step d. 7-chloro-3-(4-(2,4-difluorobenzyl)piperazin-1-yl)-2-(1-methyl-1H-pyrazol-4-yl)pyrido[3,4-b]pyrazine (Compound 193)

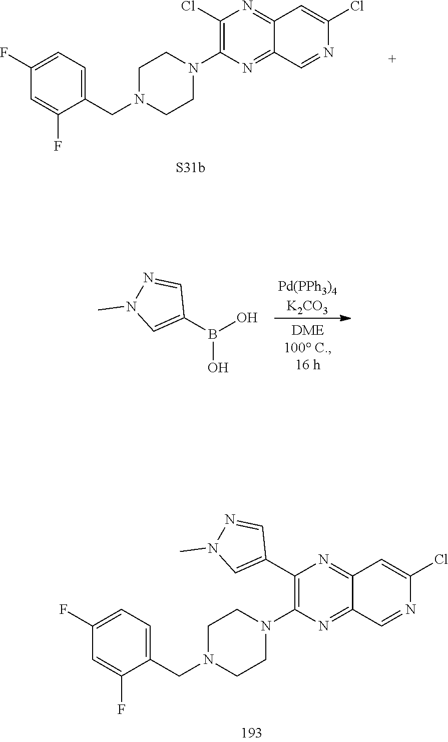

A microwave vial equipped with a stir bar was charged with S31b (300 mg, 0.731 mmol), tetrakis(triphenylphosphine)palladium(0) (86.2 mg, 0.0731 mmol), (1-methyl-1H-pyrazol-4-yl)boronic acid (99.8 mg, 0.753 mmol) and potassium carbonate (309 mg, 2.19 mmol). Then, the solids mixture was diluted in DME (3.66 mL) and the system was degassed using nitrogen gas bubbling (15 min) before sealing and heating at 100° C. overnight. The reaction mixture was filtrated on a pad of celite with EtOAc and MeOH and concentrated. The obtained crude was purified by column chromatography over silica gel using DCM/EtOAc as eluents, affording Compound 193 (206 mg, 62%) as yellow solid.

### Step g. 3-(4-(2,4-difluorobenzyl)piperazin-1-yl)-2-(1-methyl-1H-pyrazol-4-yl)-7-(2-methylprop-1-en-1-yl)pyrido[3,4-b]pyrazine (S35a)

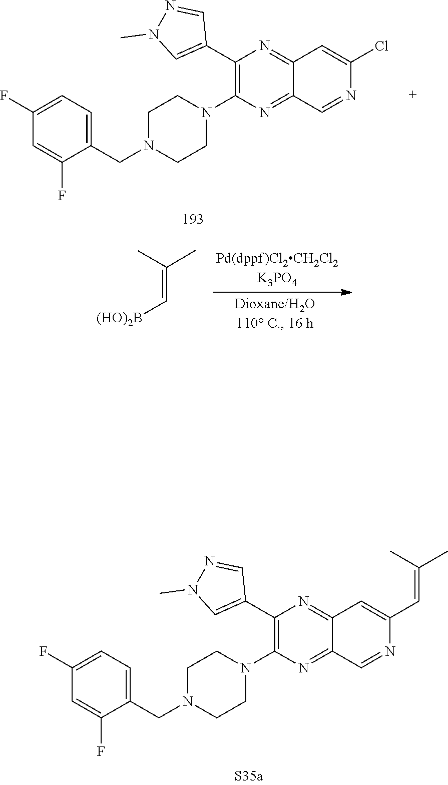

Compound S35a was synthesized following Example 001—Step d procedure using Pd(dppf)Cl₂·CH₂Cl₂ as catalyst and was obtained as yellow solid (206 mg, 65%).

### Step h. 1-(3-(4-(2,4-difluorobenzyl)piperazin-1-yl)-2-(1-methyl-1H-pyrazol-4-yl)pyrido[3,4-b]pyrazin-7-yl)-2-methylpropane-1,2-diol (Compound 197)

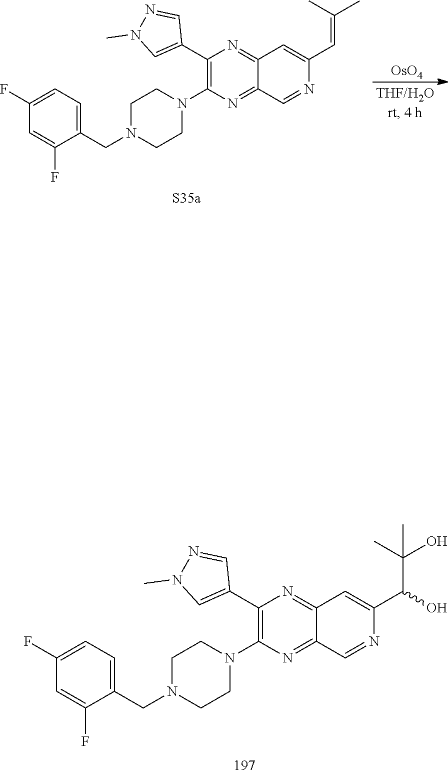

Compound 197 was synthesized following Example 021—Step e. and was obtained as brown solid (81 mg, 37%).

### Example 29. (Scheme 1) 1-(2-(4-(2,4-difluorophenoxy)piperidin-1-yl)-3-(6-(hydroxymethyl)pyridin-2-yl)-7,8-dihydropyrido[3,4-b]pyrazin-6(5H)-yl)ethan-1-one and 1-{3-[6-(2,2-difluoro-1-hydroxyethyl)pyridin-2-yl]-2-[4-(2,4-difluorophenoxy)piperidin-1-yl]-5H,6H,7H,8H-pyrido[3,4-b]pyrazin-6-yl}ethan-1-one

### Step g. 1-(2-(4-(2,4-difluorophenoxy)piperidin-1-yl)-3-(6-(hydroxymethyl)pyridin-2-yl)-7,8-dihydropyrido[3,4-b]pyrazin-6(5H)-yl)ethan-1-one (Compound 133)

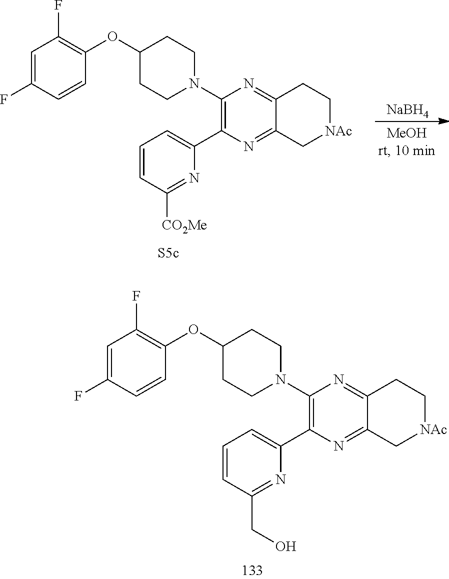

In a reaction vial, compound S5c (60.0 mg, 115 umol) was dissolved in MeOH (2 mL) and the mixture cooled down to 0° C. Then, sodium borohydride (44.2 mg, 1.15 mmol) was added. The reaction mixture was let warm to room temperature and stirred for 10 min. Afterwards, the solvents were evaporated and the crude directly purified by reverse phase column chromatography using ammonium formate/ACN as eluents to obtain Compound 133 as bright yellow fluorescent solid (25 mg, 44%).

### Step g. 6-(6-acetyl-2-(4-(2,4-difluorophenoxy)piperidin-1-yl)-5,6,7,8-tetrahydropyrido[3,4-b]pyrazin-3-yl)picolinaldehyde (S6a)

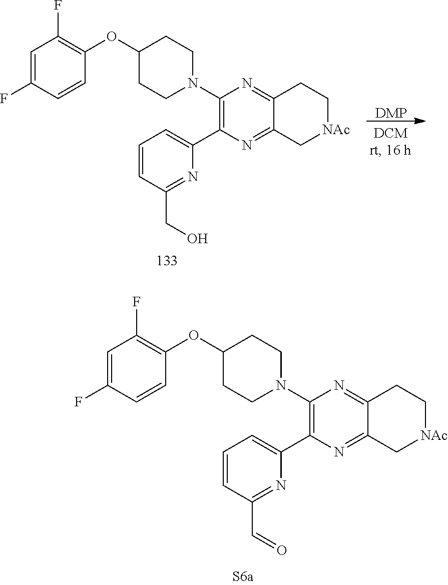

In a reaction vial, Compound 133 (65.0 mg, 131 umol) was dissolved in DCM (2 mL) and the mixture cooled down to 0° C. Then, Dess-Martin periodinane (DMP, 117 mg, 262 umol) was added. The reaction mixture was let warm to room temperature and stirred overnight. Afterwards, reaction was quenched by addition of saturated solution of sodium thiosulphate. Then, the solvents were evaporated and the crude directly purified by reverse phase column chromatography using ammonium formate/ACN as eluents to obtain S6a as bright yellow solid (50 mg, 77%).

### Step g. 1-(3-[6-(2,2-difluoro-1-hydroxyethyl)pyridin-2-yl]-2-[4-(2,4-difluorophenoxy)piperidin-1-yl]-5H,6H,7H,8H-pyrido[3,4-b]pyrazin-6-yl)ethan-1-one (Compound 210)

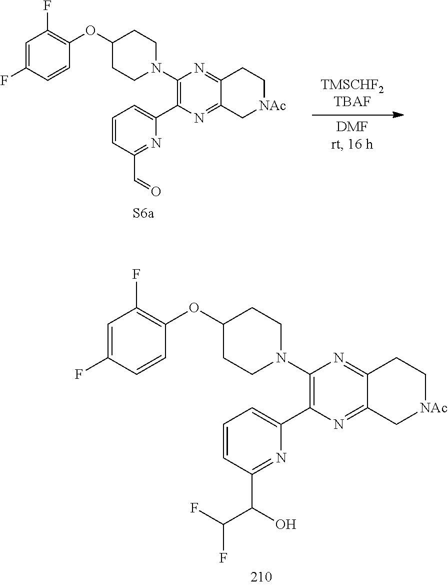

In a reaction vial, compound S6a (50.0 mg, 101 umol) was dissolved in anhydrous DMF (1 mL). Then, (difluoromethyl)trimethylsilane (51.4 mg, 405 umol) and solution of tetrabutylammonium fluoride (TBAF, 1 M in THF, 405 uL, 405 umol) were sequentially added. The reaction mixture was stirred at room temperature overnight. Afterwards, reaction was quenched by addition of 2 M HCl solution. Then, the solvents were evaporated and the crude directly purified by reverse phase column chromatography using ammonium formate/ACN as eluents to obtain Compound 210 as yellow solid (6 mg, 11%).

### Example 30. (Scheme 1) 1-(2-(4-(2,4-difluorophenoxy)piperidin-1-yl)-3-(5-fluoro-1H-pyrazol-4-yl)-7,8-dihydropyrido[3,4-b]pyrazin-6(5H)-yl)ethenone (Compound 135)

### Step d. 1-(2-(4-(2,4-difluorophenoxy)piperidin-1-yl)-3-(5-fluoro-1-((2-(trimethylsilyl)ethoxy)methyl)-1H-pyrazol-4-yl)-7,8-dihydropyrido[3,4-b]pyrazin-6(5H)-yl)ethan-1-one (S5d)

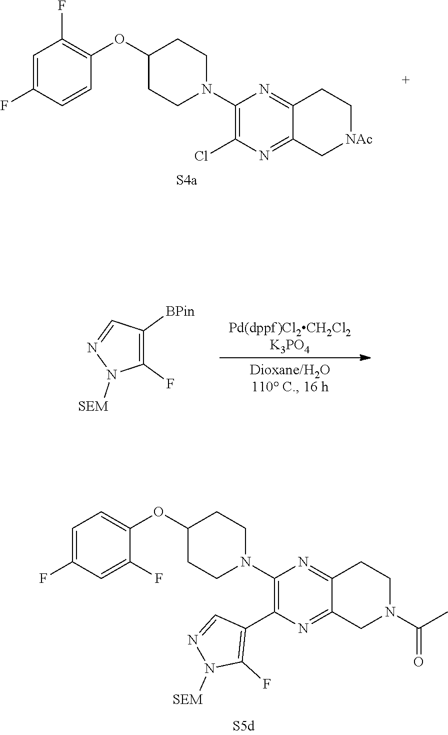

The compound was synthesized following Example 001 Step d procedure using Pd(dppf)Cl₂·CH₂Cl₂ as catalyst. S5d was obtained as dark yellow solid (135 mg, 76%).

### Step g. 1-(2-(4-(2,4-difluorophenoxy)piperidin-1-yl)-3-(5-fluoro-1H-pyrazol-4-yl)-7,8-dihydropyrido[3,4-b]pyrazin-6(5H)-yl)ethanone (Compound 135)

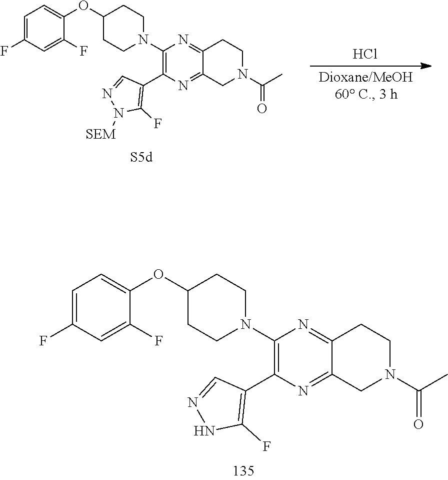 [SMILES: CC(=O)N1CCc2nc(N3CCC(Oc4ccc(F)cc4F)CC3)c(-c3cnn(COCC[Si](C)(C)C)c3F)nc2C1 ; Cl ; CC(=O)N1CCc2nc(N3CCC(Oc4ccc(F)cc4F)CC3)c(-c3cn[nH]c3F)nc2C1]

In a reaction vial, compound S5d (135 mg, 224 umol) was dissolved in a mixture of Dioxane (0.56 mL) and MeOH (0.56 mL). Then, solution of hydrochloric acid (4 M in Dioxane, 1.12 mL, 4.48 mmol) was added. The reaction mixture was heated to 60° C. room and stirred for 3 h. Afterwards, the solvents were evaporated and the crude directly purified by reverse phase column chromatography using ammonium formate/ACN as eluents to obtain Compound 135 as white solid (23.5 mg, 22%).

### Example 031. (Scheme 1) 1-(3-(6-chloropyridin-2-yl)-2-(4-(2,4-difluorophenoxy)piperidin-1-yl)-7,8-dihydropyrido[3,4-b]pyrazin-6(5H)-yl)ethenone (Compound 137) and 1-{2-[4-(2,4-difluorophenoxy)piperidin-1-yl]-3-[6-(1H-pyrazol-4-yl)pyridin-2-yl]-5H,6H,7H,8H-pyrido[3,4-b]pyrazin-6-yl}ethan-1-one (Compound 166)

### Step e. 1-(3-(6-chloropyridin-2-yl)-2-(4-(2,4-difluorophenoxy)piperidin-1-yl)-7,8-dihydropyrido[3,4-b]pyrazin-6(5H)-yl)ethanone (Compound 137)

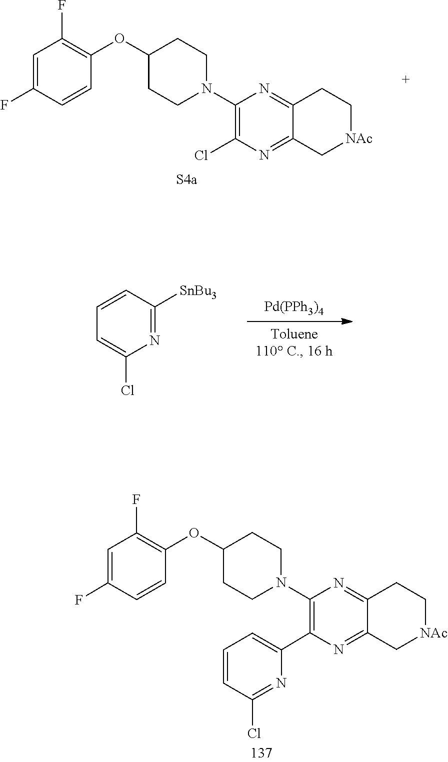

Compound 137 was synthesized following Example 002 Step e procedure and was obtained as yellow solid (110 mg, 47%).

### Step g. 1-{2-[4-(2,4-difluorophenoxy)piperidin-1-yl]-3-[6-(1H-pyrazol-4-yl)pyridin-2-yl]-5H,6H,7H,8H-pyrido[3,4-b]pyrazin-6-yl}ethan-1-one (Compound 166)

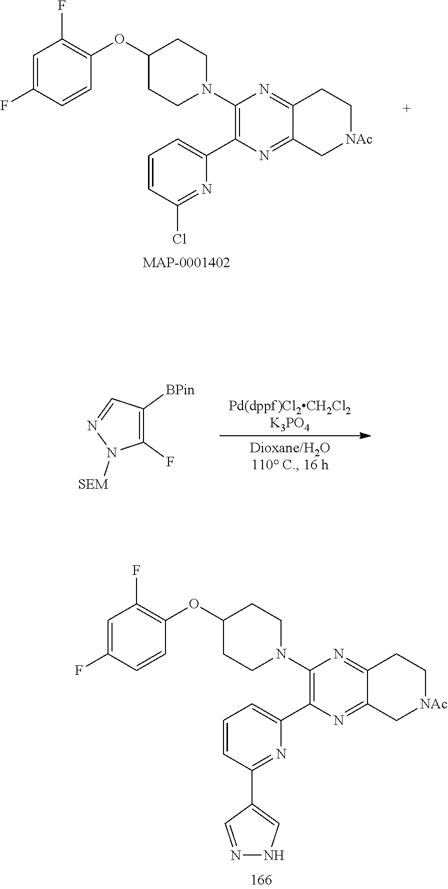

Title compound was synthesized following Example 001. Step d procedure using Pd(dppf)Cl₂·CH₂Cl₂ as catalyst. Compound 166 was obtained as yellow powder (22 mg, 59%)

### Example 032. (Scheme 1). 2-(4-(2,4-difluorophenoxy)piperidin-1-yl)-3-(1-ethyl-1H-pyrazol-4-yl)-7,8-dihydropyrido[3,4-b]pyrazine-6(5H)-carbonitrile (Compound 274)

### Step c. tert-butyl 3-chloro-2-(4-(2,4-difluorophenoxy)piperidin-1-yl)-7,8-dihydropyrido[3,4-b]pyrazine-6(5H)-carboxylate (S4b)

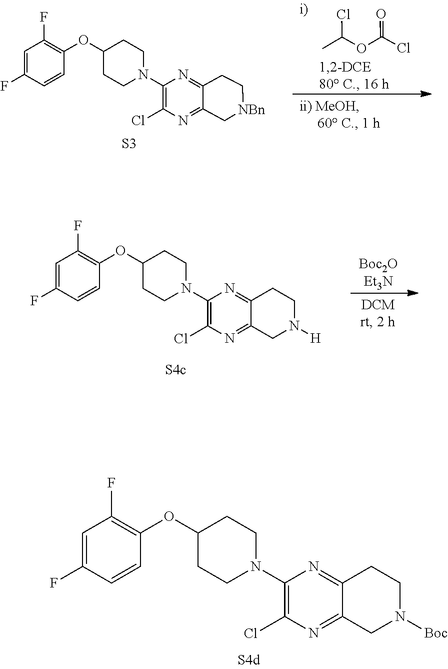

Compound S4c was synthesized following the benzyl-deprotection procedure.

Step 3. In a 25 mL round bottom flask, S4c (251 mg, 0.66 mmol) was diluted in DCM (5.0 mL) followed by the addition of triethylamine (277 uL, 1.98 mmol) and di-tert-butyl dicarbonate (155 uL, 673 umol). The mixture was stirred overnight at room temperature. Then, it was transferred to a separatory funnel using Ethyl Acetate and water. The organic phase was washed with brine and dried over sodium sulfate. Reaction crude was purified by column chromatography using Hexanes/Ethyl Acetate as eluents to obtain S4d as brown solid (317 mg, 100%).

### Step d. tert-butyl 2-(4-(2,4-difluorophenoxy)piperidin-1-yl)-3-(1-ethyl-1H-pyrazol-4-yl)-7,8-dihydropyrido[3,4-b]pyrazine-6(5H)-carboxylate (S5e)

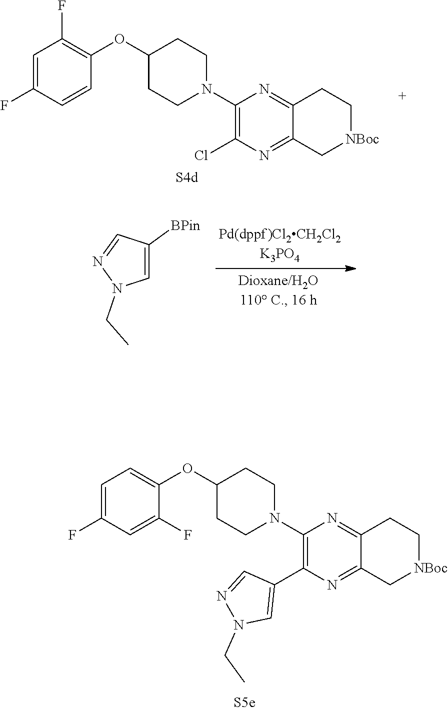

Compound S5e was synthesized following Example 001 Step d procedure using Pd(dppf)Cl₂·CH₂Cl₂ as catalyst and was obtained as yellow solid (266 mg, 75%).

### Step g. 2-(4-(2,4-difluorophenoxy)piperidin-1-yl)-3-(1-ethyl-1H-pyrazol-4-yl)-7,8-dihydropyrido[3,4-b]pyrazine-6(5H)-carbonitrile (Compound 274)

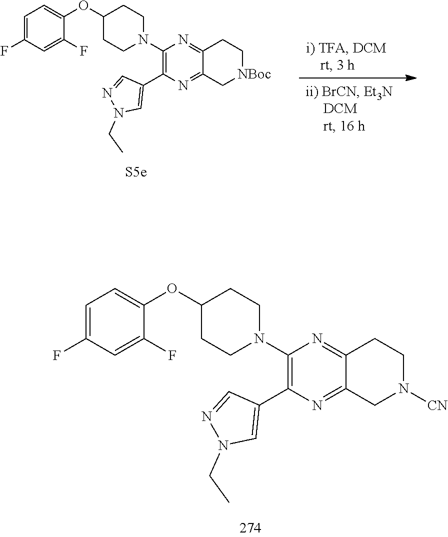 [SMILES: CCn1cc(-c2nc3c(nc2N2CCC(Oc4ccc(F)cc4F)CC2)CCN(C(=O)OC(C)(C)C)C3)cn1 ; CCn1cc(-c2nc3c(nc2N2CCC(Oc4ccc(F)cc4F)CC2)CCN(C#N)C3)cn1]

Step 1. To a solution of amine S5e (53.0 mg, 94.3 umol) in DCM (1.0 mL) was added trifluoroacetic acid (500 uL, 6.46 mmol) at room temperature. The system was stirred for 2 h at same temperature. After the full consumption of starting material, the excess TFA was removed under vacuum and azeotrope with DCM (3×5.0 mL) and dried further under vacuum. Step 2. The obtained reaction crude was dissolved in DCM (2.0 mL) and cooled to 0° C. Then, triethylamine (68.7 uL, 490 umol) and cyanogen bromide (21.4 mg, 196 umol) were sequentially added and the mixture stirred overnight at room temperature. Then, the reaction mixture was concentrated under a vacuum to remove excess solvents and reagents. Afterwards, the obtained crude was purified by reverse phase column chromatography using ammonium formate/ACN as eluents as eluents to give Compound 274 (16.6 mg, 36%) as yellow solid.

### Example 033. (Scheme 2). 4-(2,4-difluorophenoxy)-4-methyl-1-[3-(1-methyl-1H-pyrazol-4-yl)pyrido[3,4-b]pyrazin-2-yl]piperidine (Compound 188)

### Step a. tert-butyl 4-(2,4-difluorophenoxy)-4-(((methylsulfonyl)oxy)methyl)piperidine-1-carboxylate (S9g)

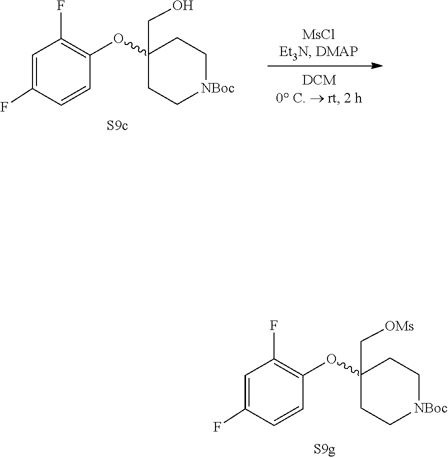 [SMILES: CC(C)(C)OC(=O)N1CCC(CO)(Oc2ccc(F)cc2F)CC1 ; CC(C)(C)OC(=O)N1CCC(COS(C)(=O)=O)(Oc2ccc(F)cc2F)CC1]

In a 50 mL round bottom flask, a solution of S9c (1.05 g, 3.06 mmol) in anhydrous DCM (20 mL) was cooled down to 0° C. Then, triethylamine (861 uL, 6.12 mmol), 4-dimethylaminopyridine (DMAP, 19.1 mg, 153 umol), and methanesulfonyl chloride (285 uL, 3.67 mmol) were sequentially added. After addition, the mixture was stirred at room temperature for 2 h. The reaction mixture was quenched by addition of water and it was extracted with Ethyl Acetate (3×). The combined organic layers were washed with brine, dried over sodium sulfate and evaporated under reduced pressure. The obtained crude (1.10 g, 85%) was used in the next steps of the synthesis without additional purification.

### Step a. tert-butyl 4-(2,4-difluorophenoxy)-4-methylpiperidine-1-carboxylate (S9h)

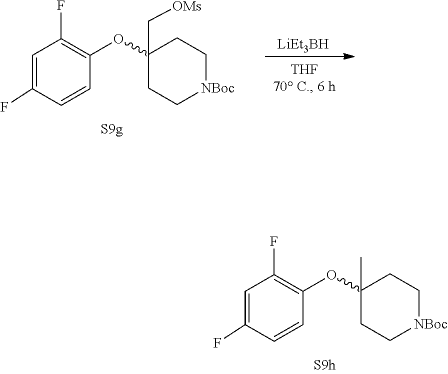 [SMILES: CC(C)(C)OC(=O)N1CCC(COS(C)(=O)=O)(Oc2ccc(F)cc2F)CC1 ; CC(C)(C)OC(=O)N1CCC(C)(Oc2ccc(F)cc2F)CC1 ; "LiEt3BH"]

In a 100 mL round bottom flask under nitrogen atmosphere, compound S9g (1.10 g, 2.61 mmol) was dissolved in anhydrous THF (17.1 mL). Then, a solution of lithium triethylborohydride (1 M in THF, 10.4 mL, 10.4 mmol) was added. After addition, the mixture was heated to 70° C. and stirred for 6 h (complete reaction indicated by LCMS analysis). The reaction mixture was quenched by addition of water and it was extracted with Ethyl Acetate (3×). The combined organic layers were washed with brine, dried over sodium sulfate and evaporated under reduced pressure. The obtained residue was purified by silica gel chromatography using Hexanes/Ethyl Acetate as eluents to obtain compound S9h (88.0 mg, 10%) as a colorless oil.

### Step b. 3-chloro-2-(4-(2,4-difluorophenoxy)-4-methylpiperidin-1-yl)pyrido[3,4-b]pyrazine (S10e)

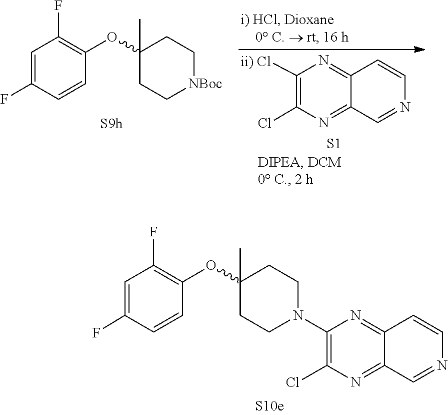

Compound S10e was synthesized following Example 001 Step d. and was obtained as dark brown solid (93 mg, 76%).

### Step c. 4-(2,4-difluorophenoxy)-4-methyl-1-[3-(1-methyl-1H-pyrazol-4-yl)pyrido[3,4-b]pyrazin-2-yl]piperidine (Compound 188)

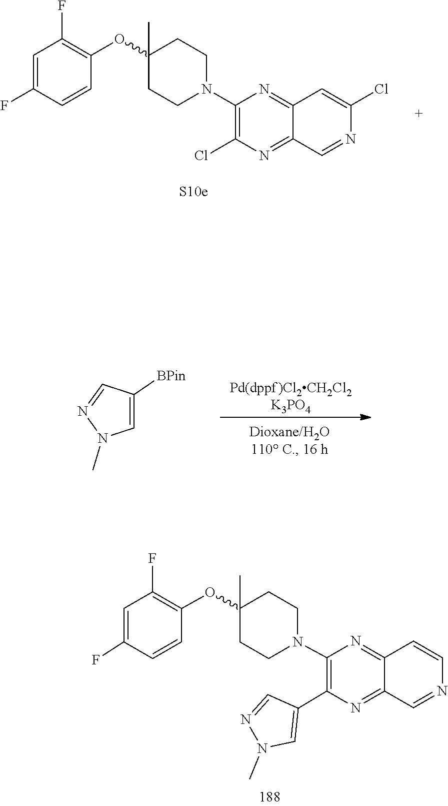

Compound 188 was synthesized following Example 001. Step d procedure using Pd(dppf)Cl₂·CH₂Cl₂ as catalyst. Compound 188 was obtained as off-yellow solid (26.2 mg, 24%).

### Example 034. (Scheme 3) (2,4-difluorophenyl)(4-(3-(1-methyl-1H-pyrazol-4-yl)pyrido[3,4-b]pyrazin-2-yl)piperazin-1-yl)methanone (Compound 128)

### Step a. tert-butyl 4-(2,4-difluorobenzoyl)piperazine-1-carboxylate (S15a)

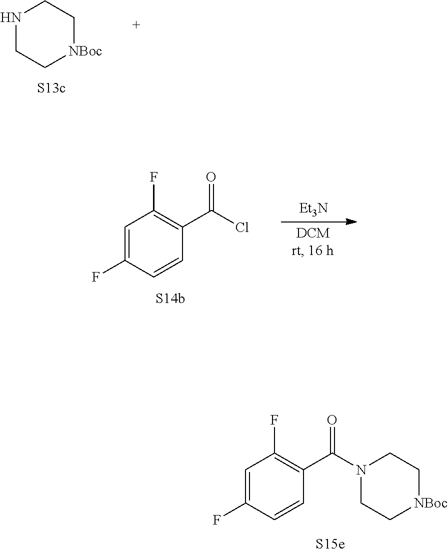 [SMILES: "Et3N" ; CC(C)(C)OC(=O)N1CCNCC1 ; O=C(Cl)c1ccc(F)cc1F ; CC(C)(C)OC(=O)N1CCN(C(=O)c2ccc(F)cc2F)CC1]

In a 50 mL round bottom flask, a solution of 1-boc-piperazine (S13c, 500 mg, 2.63 mmol) and triethylamine (741 uL, 5.26 mmol) in DCM (10.5 mL) was cooled down to 0° C. Then, 2,4-difluorobenzoyl chloride (S14b, 327 μL, 2.63 mmol) was added dropwise. The reaction mixture was let warm to room temperature and stirred overnight. Afterwards, reaction was quenched by the addition of saturated sodium bicarbonate solution. Then, the reaction crude was transferred to a separatory funnel using DCM/water and extracted with DCM (3×). The organic phase was dried over sodium sulphate, filtered and concentrated. The reaction crude was purified by column chromatography using Heptanes/Ethyl Acetate as eluents, to obtain compound S15e was obtained as colorless oil (790 mg, 92%).

### Step b. (4-(3-chloropyrido[3,4-b]pyrazin-2-yl)piperazin-1-yl)(2,4-difluorophenyl)methanone (S16d)

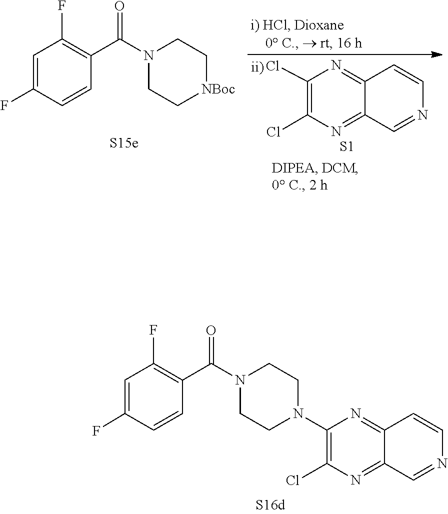

Compound S16d was synthesized as yellow solid (369 mg, 83%) following Example 001. Step d.

### Step c. (2,4-difluorophenyl)(4-(3-(1-methyl-1H-pyrazol-4-yl)pyrido[3,4-b]pyrazin-2-yl)piperazin-1-yl)methanone (Compound 128)

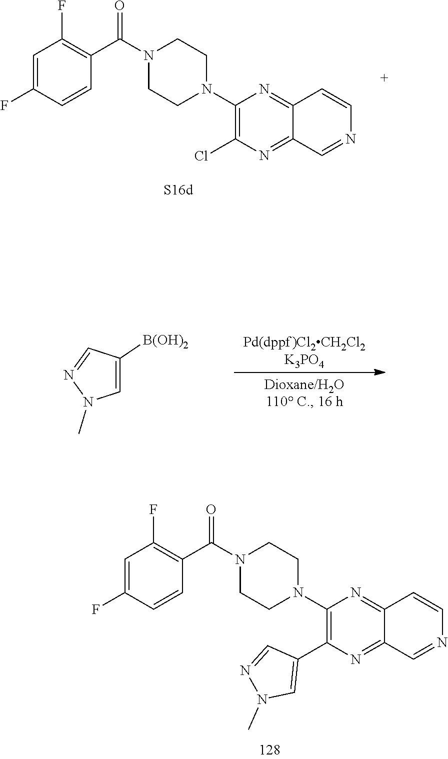

Compound 128 was synthesized as yellow powder (158 mg, 77%) following Example 001. Step d procedure using Pd(dppf)Cl₂·CH₂Cl₂ as catalyst.

### Example 035. (Scheme 3) 4-(2,4-difluorobenzyl)-1-(3-(1-methyl-1H-pyrazol-4-yl)pyrido[3,4-b]pyrazin-2-yl)piperazin-2-one (Compound 259)

### Step a. tert-butyl 4-(2,4-difluorobenzyl)-2-oxopiperazine-1-carboxylate (S15f)

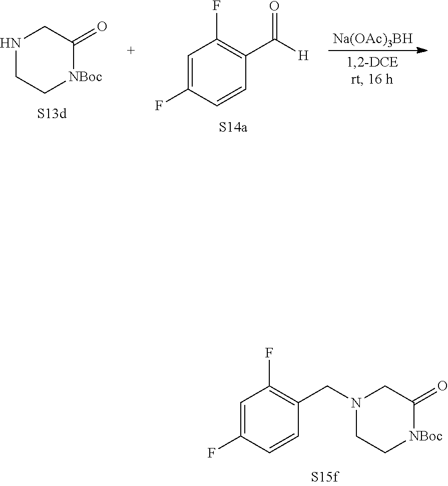 [SMILES: CC(C)(C)OC(=O)N1CCNCC1=O ; O=Cc1ccc(F)cc1F ; CC(C)(C)OC(=O)N1CCN(Cc2ccc(F)cc2F)CC1=O ; "Na(OAc)3BH"]

S15f was synthesized following reductive amination procedure in Example 001. Step d. 15b was obtained as white solid (296 mg, 96%).

### Step b. 1-(3-chloropyrido[3,4-b]pyrazin-2-yl)-4-(2,4-difluorobenzyl)piperazin-2-one (S16d)

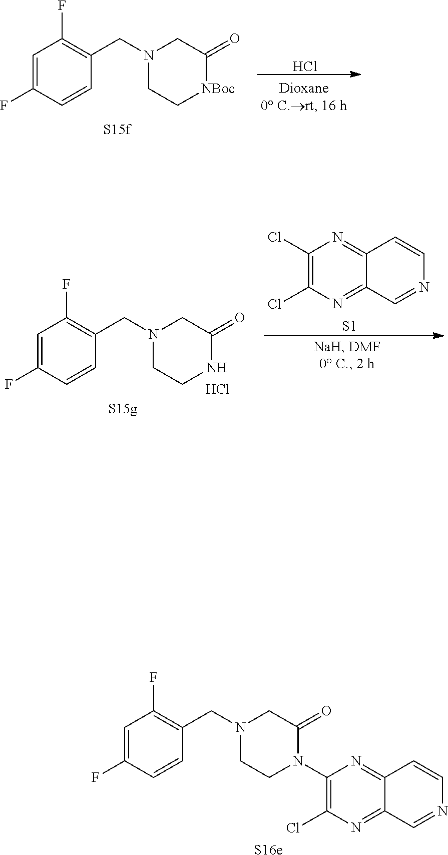

Step 1. Following the boc-deprotection procedure. S15g was obtained as a white solid. Step 2. In a flame-dried round bottom flask under nitrogen, sodium hydride (60% suspension in mineral oil, 23.0 mg, 575 umol) was added to a solution of S15g (100 mg, 442 umol) in dry DMF (2.24 mL) at 0° C. The mixture was allowed to stir for 1 h at 0° C. Then, the system was quickly opened and 2,3-dichloropyrido[3,4-b]pyrazine (S1, 99.2 mg, 486 umol) was added and the mixture was let warm to room temperature under nitrogen for 1 h. Afterwards, reaction was quenched by the addition of saturated sodium bicarbonate solution. Then, the reaction crude was transferred to a separatory funnel using Ethyl Acetate/water and extracted with Ethyl Acetate (3×). The organic phase was dried over sodium sulphate, filtered and concentrated. The reaction crude was purified by reverse phase column chromatography using ammonium formate/ACN as eluents, compound S16e was obtained as yellow solid (74 mg, 43%).

### Step c. 4-(2,4-difluorobenzyl)-1-(3-(1-methyl-1H-pyrazol-4-yl)pyrido[3,4-b]pyrazin-2-yl)piperazin-2-one (Compound 259)

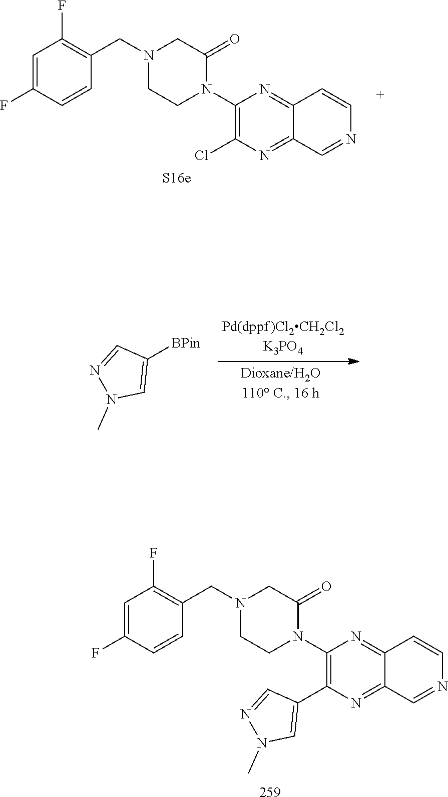

Compound 259 was synthesized following Example 001. Step d procedure using Pd(dppf)Cl₂·CH₂Cl₂ as catalyst and was obtained as white powder (42 mg, 25%).

### Example 36. (Scheme 1) 6-(6-acetyl-2-(4-(2,4-difluorophenoxy)piperidin-1-yl)-5,6,7,8-tetrahydropyrido[3,4-b]pyrazin-3-yl)-N,N-dimethylpicolinamide (Compound 131)

### Step e. methyl 6-(6-acetyl-2-(4-(2,4-difluorophenoxy)piperidin-1-yl)-5,6,7,8-tetrahydropyrido[3,4-b]pyrazin-3-yl)picolinate (S5c)

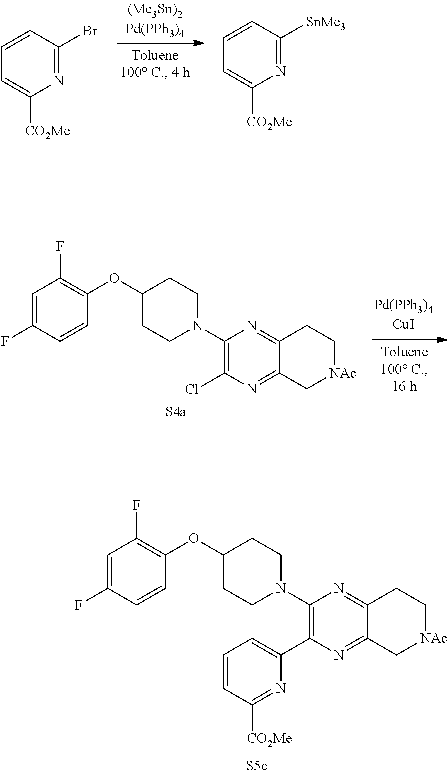

Compound 131 was synthesized following Example 002 Step e procedure. S5c was obtained as bright yellow solid (500 mg, 82%).

### Step g. 6-(6-acetyl-2-(4-(2,4-difluorophenoxy)piperidin-1-yl)-5,6,7,8-tetrahydropyrido[3,4-b]pyrazin-3-yl)-N,N-dimethylpicolinamide

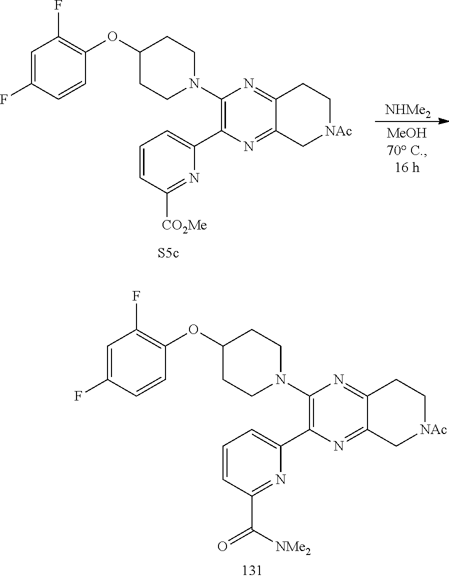

In a reaction vial, compound S5c (60.0 mg, 115 umol) was dissolved in in MeOH (0.5 mL). Then, solution of dimethylamine (33% in EtOH, 461 uL, 2.29 mmol) was added. The reaction mixture was heated to 70° C. until and stirred overnight. Afterwards, the solvents were evaporated and the crude directly purified by reverse phase column chromatography using ammonium bicarbonate/ACN as eluents to obtain Compound 131 as bright yellow fluorescent solid (25 mg, 41%).

### Example 37. (Scheme 1) 4-(6-acetyl-2-(4-(2,4-difluorophenoxy)piperidin-1-yl)-5,6,7,8-tetrahydropyrido[3,4-b]pyrazin-3-yl)cyclohexan-1-one (Compound 404), 1-(2-(4-(2,4-difluorophenoxy)piperidin-1-yl)-3-(4-hydroxycyclohexyl)-7,8-dihydropyrido[3,4-b]pyrazin-6(5H)-yl)ethan-1-one (Compound 374) and 1-(2-(4-(2,4-difluorophenoxy)piperidin-1-yl)-3-(4-methoxycyclohexyl)-7,8-dihydropyrido[3,4-b]pyrazin-6(5H)-yl)ethan-1-one (Compound 373)

### Step d. 1-(2-(4-(2,4-difluorophenoxy)piperidin-1-yl)-3-(1,4-dioxaspiro[4.5]dec-7-en-8-yl)-7,8-dihydropyrido[3,4-b]pyrazin-6(5H)-yl)ethan-1-one (S5f)

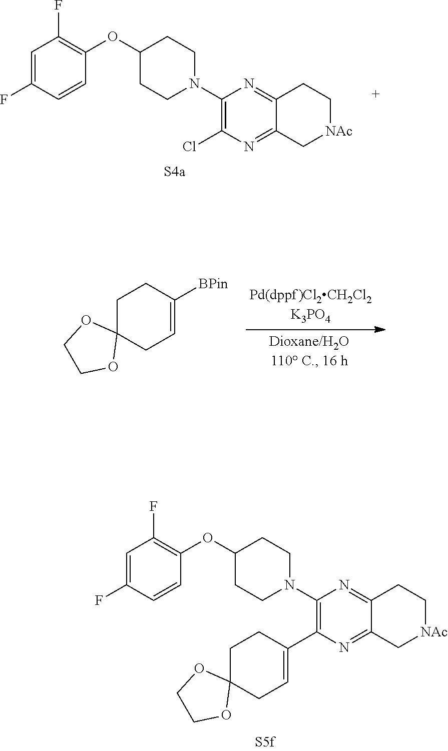

Compound 404 was synthesized following Example 37 Step d procedure using Pd(dppf)Cl₂·CH₂Cl₂ as catalyst. Sf was obtained as yellow solid (62%).

### Step g. 4-(6-acetyl-2-(4-(2,4-difluorophenoxy)piperidin-1-yl)-5,6,7,8-tetrahydropyrido[3,4-b]pyrazin-3-yl)cyclohexan-1-one (Compound 404)

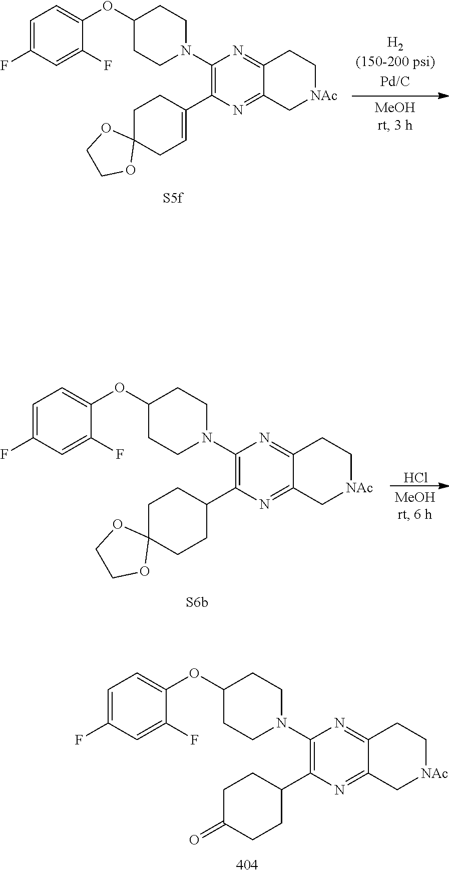

Step 1. Compound S6b was synthesized following Example 009. Step g procedure using Pd/C as catalyst. Crude S6b was used directly in the next step without purification.

Step 2. In a 25 mL round bottom flask, a solution of HCl (3 N in MeOH, 5.0 mL) was added to crude S6b (157 mg, 297 umol) at room temperature and the mixture stirred for 6 h. Then, the reaction was quenched by adding saturated solution of NaHCO₃. The obtained mixture was loaded directly into the C18 silica column and purified by reverse phase column chromatography using ammonium formate/ACN as eluents as eluents to give compound Compound 404 (84 mg, 58%) as white solid.

### Step g. 1-(2-(4-(2,4-difluorophenoxy)piperidin-1-yl)-3-(4-hydroxycyclohexyl)-7,8-dihydropyrido[3,4-b]pyrazin-6(5H)-yl)ethan-1-one (Compound 374)

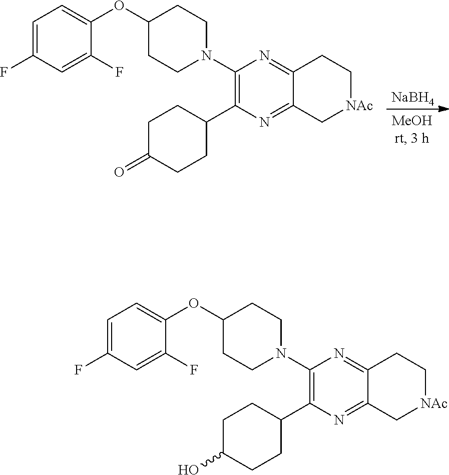

Compound 374 was synthesized following reduction procedure in Example 029. Step g. and was obtained as yellow solid (22 mg, 47%).

### Step g. 1-(2-(4-(2,4-difluorophenoxy)piperidin-1-yl)-3-(4-methoxycyclohexyl)-7,8-dihydropyrido[3,4-b]pyrazin-6(5H)-yl)ethan-1-one (Compound 373)

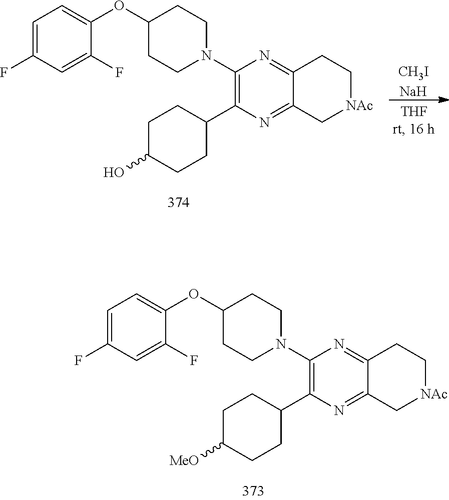

In a 25 mL round bottom flask, Compound 374 (15.0 mg, 30.8 umol) was dissolved in anhydrous THF (1.50 mL). Then, sodium hydride (60% dispersion in mineral oil, 2.47 mg, 61.7 umol) and iodomethane (9.69 uL, 154 umol) were sequentially added at room temperature and the mixture stirred overnight. Afterwards, the reaction was quenched by adding saturated solution of NH₄Cl. The obtained mixture was loaded directly into the C18 silica column and purified by reverse phase column chromatography using ammonium formate/ACN as eluents as eluents to give Compound 373 (84 mg, 58%) as white solid.

### Example 38. (Scheme 1) 1-(2-(4-(2,4-difluorophenoxy)piperidin-1-yl)-3-(2-(hydroxymethyl)cyclopropyl)-7,8-dihydropyrido[3,4-b]pyrazin-6(5H)-yl)ethan-1-one (Compound 489) and 1-(2-(4-(2,4-difluorophenoxy)piperidin-1-yl)-3-(2-(methoxymethyl)cyclopropyl)-7,8-dihydropyrido[3,4-b]pyrazin-6(5H)-yl)ethan-1-one (Compound 463)

### Step d. methyl 3-(6-acetyl-2-(4-(2,4-difluorophenoxy)piperidin-1-yl)-5,6,7,8-tetrahydropyrido[3,4-b]pyrazin-3-yl)acrylate (S5g)

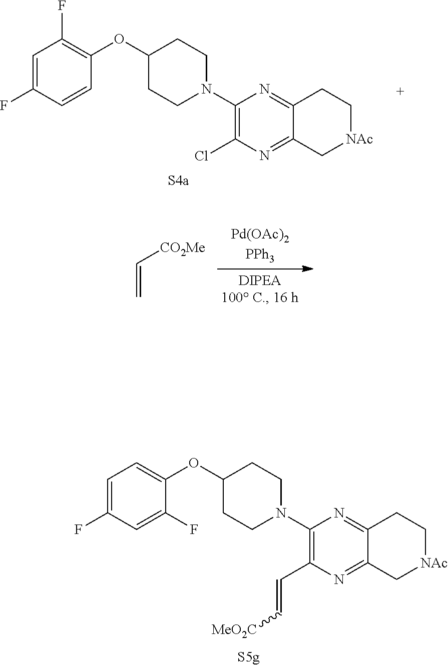

A microwave vial equipped with a stir bar was charged with S4a (150 mg, 355 umol), palladium acetate (8.13 mg, 35.5 umol) and triphenylphosphine (18.8 mg, 70.9 umol) and the solids were diluted in methyl acrylate (1.00 mL) and then degassed by nitrogen gas bubbling (5 min). Then, the container was sealed and the mixture heated to 100° C. overnight. Afterwards, the reaction mixture was filtered through Celite and the solvents evaporated under reduced pressure. Reaction crude was purified by reverse phase column chromatography using ammonium formate/ACN as eluents to obtain S5g as yellow fluorescent solid (141 mg, 84%).

### Step g. methyl 2-(6-acetyl-2-(4-(2,4-difluorophenoxy)piperidin-1-yl)-5,6,7,8-tetrahydropyrido[3,4-b]pyrazin-3-yl)cyclopropane-1-carboxylate (S6c)

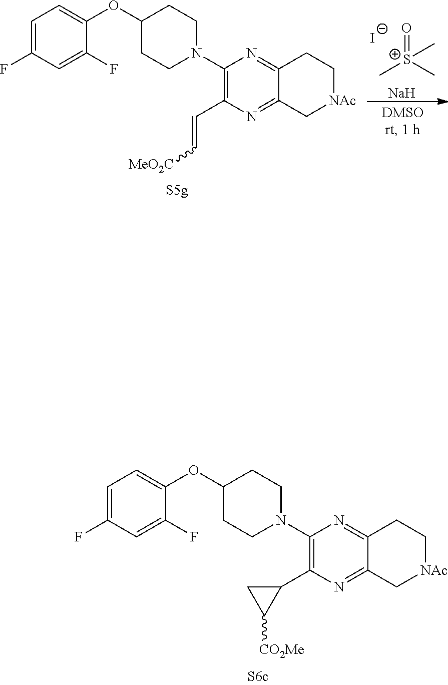

In a 25 ml round bottom flask, trimethyl sulfoxonium iodide (85.4 mg, 372 umol) was dissolved in DMSO (3.39 mL), followed by addition of sodium hydride (10.6 mg, 440 umol). The mixture was stirred at room temperature for 15 min, then, S5g (70.0 mg, 148 umol) was added to the mixture and stirred for 1 h at room temperature (complete conversion indicated by LCMS analysis). Afterwards, the reaction was quenched but the addition of water and it was transferred to a separatory funnel using Ethyl Acetate and water. The organic phase was washed with brine and dried over sodium sulfate. Reaction crude was purified by reverse phase column chromatography using ammonium formate/ACN as eluents to obtain S6d as off-white solid (50 mg, 30%).

### Step g. 1-(2-(4-(2,4-difluorophenoxy)piperidin-1-yl)-3-(2-(hydroxymethyl)cyclopropyl)-7,8-dihydropyrido[3,4-b]pyrazin-6(5H)-yl)ethan-1-one (Compound 489)

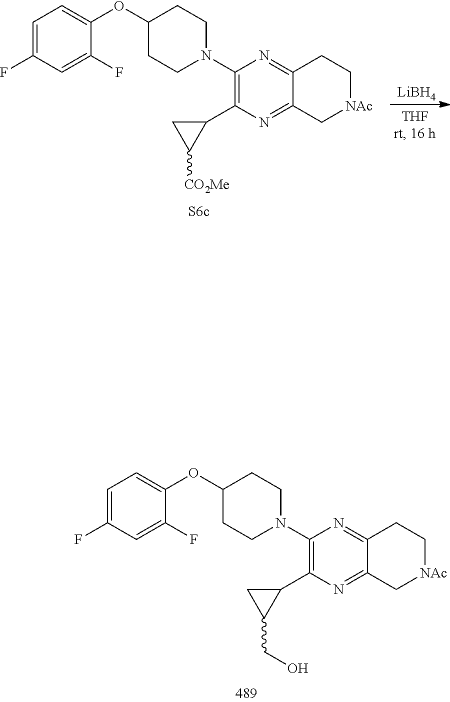

In a reaction vial, compound S6c (50.0 mg, 103 umol) was dissolved in THF (1.11 mL) and the mixture cooled down to 0° C. Then, lithium borohydride (44.2 mg, 1.15 mmol) was added. The reaction mixture was let warm to room temperature and stirred overnight. Afterwards, the solvents were evaporated and the crude directly purified by reverse phase column chromatography using ammonium formate/ACN as eluents to obtain Compound 489 as off-white solid (40.0 mg, 85%).

### Step 2. 1-(2-(4-(2,4-difluorophenoxy)piperidin-1-yl)-3-(2-(methoxymethyl)cyclopropyl)-7,8-dihydropyrido[3,4-b]pyrazin-6(5H)-yl)ethan-1-one (Compound 463)

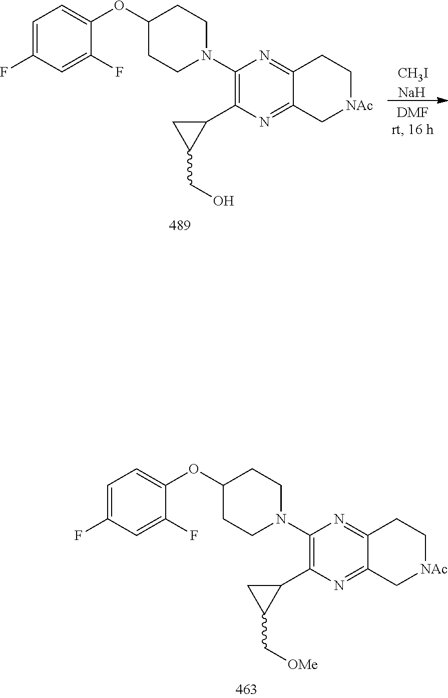

Compound 463 was synthesized following methylation procedure in Example 037. Step g. and was obtained as off-white solid (10 mg, 32%).

### Example 39. (Scheme 1) 2-(4-(2,4-difluorophenoxy)piperidin-1-yl)-3-(1-ethyl-1H-pyrazol-4-yl)-7,8-dihydropyrido[3,4-b]pyrazine-6(5H)-sulfonamide (Compound 478)

### Step d. 6-benzyl-2-(4-(2,4-difluorophenoxy)piperidin-1-yl)-3-(1-ethyl-1H-pyrazol-4-yl)-5,6,7,8-tetrahydropyrido[3,4-b]pyrazine (S5h)

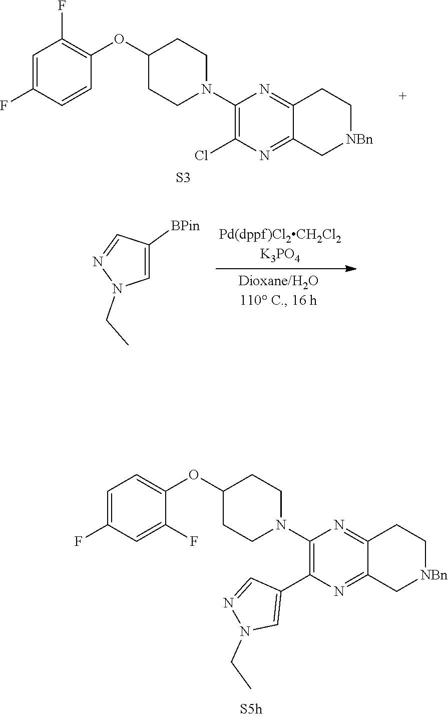

Compound S5h was synthesized following Example 001. Step d procedure using Pd(dppf)Cl₂·CH₂Cl₂ as catalyst and was obtained as brown gum (1.30 g, 89%).

### Step c. 2-(4-(2,4-difluorophenoxy)piperidin-1-yl)-3-(1-ethyl-1H-pyrazol-4-yl)-5,6,7,8-tetrahydropyrido[3,4-b]pyrazine (S4e)

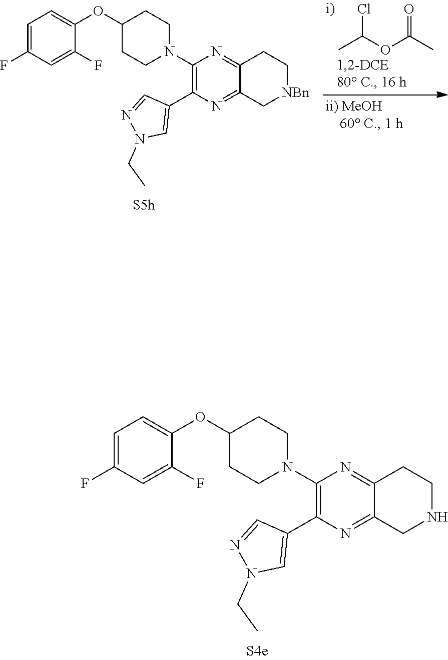

Compound S4e was synthesized following benzyl-deprotection and was obtained as yellow gum (480 mg, 44%).

### Step g. 2-(4-(2,4-difluorophenoxy)piperidin-1-yl)-3-(1-ethyl-1H-pyrazol-4-yl)-7,8-dihydropyrido[3,4-b]pyrazine-6(5H)-sulfonamide (Compound 478)

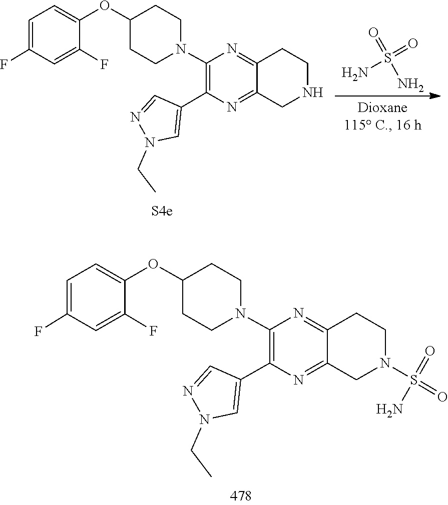 [SMILES: CCn1cc(-c2nc3c(nc2N2CCC(Oc4ccc(F)cc4F)CC2)CCNC3)cn1 ; NS(N)(=O)=O ; CCn1cc(-c2nc3c(nc2N2CCC(Oc4ccc(F)cc4F)CC2)CCN(S(N)(=O)=O)C3)cn1]

In a reaction vial, compound S4e (30.0 mg, 68.1 umol) was dissolved in dioxane (1.00 mL). Then, sulfamide (8.02 mg, 81.7 umol) was added. The reaction mixture was heated to 115° C. and stirred overnight. Afterwards, the solvents were evaporated and the crude directly purified by reverse phase column chromatography using ammonium bicarbonate/ACN as eluents to obtain Compound 478 as off-white solid (14.0 mg, 40%).

### Example 40. (Scheme 1) (2-(4-(2,4-difluorophenoxy)piperidin-1-yl)-3-(1-ethyl-1H-pyrazol-4-yl)-7,8-dihydropyrido[3,4-b]pyrazin-6(5H)-yl)(4,4-difluoropyrrolidin-2-yl)methanone (Compound 481) and (4,4-difluoro-1-methylpyrrolidin-2-yl)(2-(4-(2,4-difluorophenoxy)piperidin-1-yl)-3-(1-ethyl-1H-pyrazol-4-yl)-7,8-dihydropyrido[3,4-b]pyrazin-6(5H)-yl)methanone (Compound 483)

### Step g. tert-butyl 2-(2-(4-(2,4-difluorophenoxy)piperidin-1-yl)-3-(1-ethyl-1H-pyrazol-4-yl)-5,6,7,8-tetrahydropyrido[3,4-b]pyrazine-6-carbonyl)-4,4-difluoropyrrolidine-1-carboxylate (S4f)

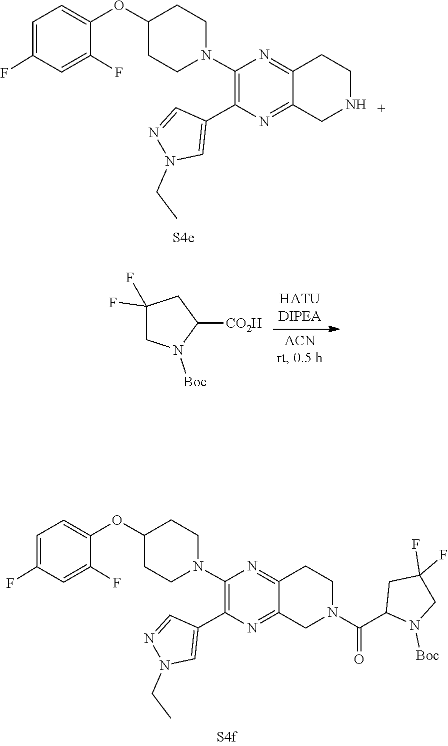 [SMILES: CCn1cc(-c2nc3c(nc2N2CCC(Oc4ccc(F)cc4F)CC2)CCNC3)cn1 ; CC(C)(C)OC(=O)N1CC(F)(F)CC1C(=O)O ; CCn1cc(-c2nc3c(nc2N2CCC(Oc4ccc(F)cc4F)CC2)CCN(C(=O)C2CC(F)(F)CN2C(=O)OC(C)(C)C)C3)cn1]

In a reaction vial, compound S4e (100 mg, 227 umol) was dissolved in ACN (2.00 mL). Then, 1-(tert-butoxycarbonyl)-4,4-difluoropyrrolidine-2-carboxylic acid (75.9 uL, 227 umol), N,N-diisopropylethylamine (160 uL, 908 umol) and HATU (176 mg, 454 umol) were sequentially added. The reaction mixture was stirred at room temperature for 30 min (complete conversion indicated by LCMS analysis). Afterwards, the reaction was diluted with ethyl acetate and transferred to a separatory funnel. The organic phase was washed with water and dried over sodium sulfate. Reaction crude was purified by reverse phase column chromatography using ammonium formate/ACN as eluents to obtain S4f as brown oil (181 mg, quant.).

### Step g. (2-(4-(2,4-difluorophenoxy)piperidin-1-yl)-3-(1-ethyl-1H-pyrazol-4-yl)-7,8-dihydropyrido[3,4-b]pyrazin-6(5H)-yl)(4,4-difluoropyrrolidin-2-yl)methanone (Compound 481)

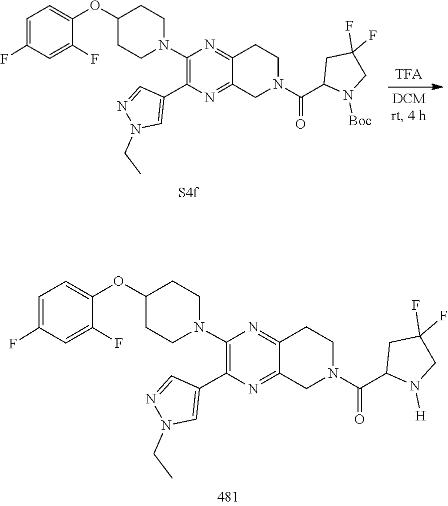 [SMILES: CCn1cc(-c2nc3c(nc2N2CCC(Oc4ccc(F)cc4F)CC2)CCN(C(=O)C2CC(F)(F)CN2C(=O)OC(C)(C)C)C3)cn1 ; CCn1cc(-c2nc3c(nc2N2CCC(Oc4ccc(F)cc4F)CC2)CCN(C(=O)C2CC(F)(F)CN2)C3)cn1]

Compound 481 was synthesized following Boc-deprotection procedure and was obtained as white solid (52 mg, 40%).

### Step g. (4,4-difluoro-1-methylpyrrolidin-2-yl)(2-(4-(2,4-difluorophenoxy)piperidin-1-yl)-3-(1-ethyl-1H-pyrazol-4-yl)-7,8-dihydropyrido[3,4-b]pyrazin-6(5H)-yl)methanone (Compound 483)

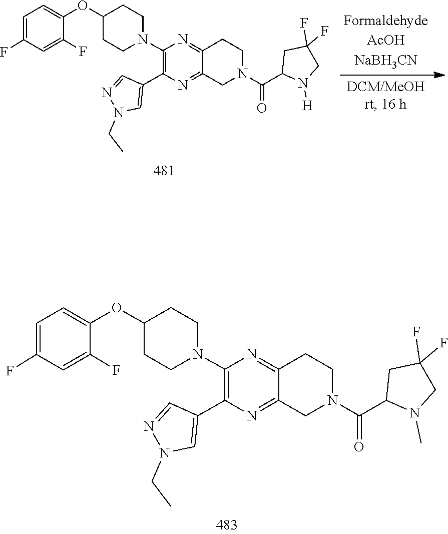 [SMILES: CCn1cc(-c2nc3c(nc2N2CCC(Oc4ccc(F)cc4F)CC2)CCN(C(=O)C2CC(F)(F)CN2)C3)cn1 ; CCn1cc(-c2nc3c(nc2N2CCC(Oc4ccc(F)cc4F)CC2)CCN(C(=O)C2CC(F)(F)CN2C)C3)cn1]

Compound 483 was synthesized following reductive amination procedure and was obtained as white solid (5.55 mg, 54%).

### Example 41. (Scheme 1): 2-(4-(2,4-difluorophenoxy)piperidin-1-yl)-3-(1-ethyl-1H-pyrazol-4-yl)-6-(S-methylsulfonimidoyl)-5,6,7,8-tetrahydropyrido[3,4-b]pyrazine (Compound 485)

### Step g. 2-(4-(2,4-difluorophenoxy)piperidin-1-yl)-3-(1-ethyl-1H-pyrazol-4-yl)-6-(S-methylsulfonimidoyl)-5,6,7,8-tetrahydropyrido[3,4-b]pyrazine (Compound 485)

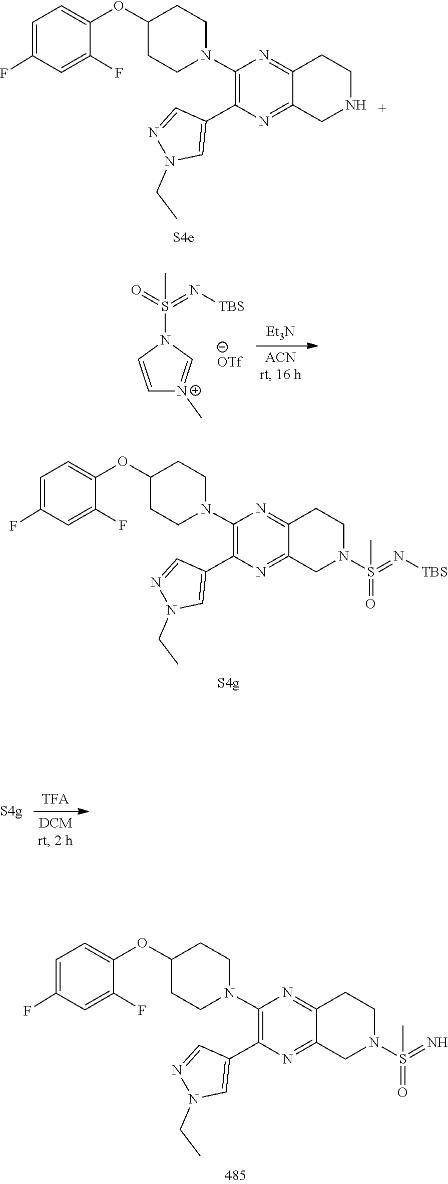

Step 1. In a reaction vial, compound S4 (30.0 mg, 68.1 umol) was dissolved in ACN (1.50 mL). Then, 1-{[(tert-butyldimethylsilyl)imino](methyl)oxo-lambda6-sulfanyl}-3-methyl-1H-imidazol-3-ium trifluoromethanesulfonate (18.8 uL, 74.9 umol) and triethylamine (47.7 uL, 341 umol) were sequentially added. The reaction mixture was stirred at room temperature overnight. Afterwards, the reaction was diluted with ethyl acetate and transferred to a separatory funnel. The organic phase was washed with water and dried over sodium sulfate. Reaction crude was used directly in the next step.

Step 2. Reaction crude was submitted to deprotection procedure and Compound 485 was obtained as white solid (15.3 mg, 43%).

### Example 42. (Scheme 4) (Rel-S)-1-(2-(4-(2,4-difluorophenoxy)piperidin-1-yl)-3-(1-methyl-1H-pyrazol-4-yl)pyrido[3,4-b]pyrazin-7-yl)-2-methylpropane-1,2-diol (Compound 378) and (Rel-R)-1-(2-(4-(2,4-difluorophenoxy)piperidin-1-yl)-3-(1-methyl-1H-pyrazol-4-yl)pyrido[3,4-b]pyrazin-7-yl)-2-methylpropane-1,2-diol (Compound 379)

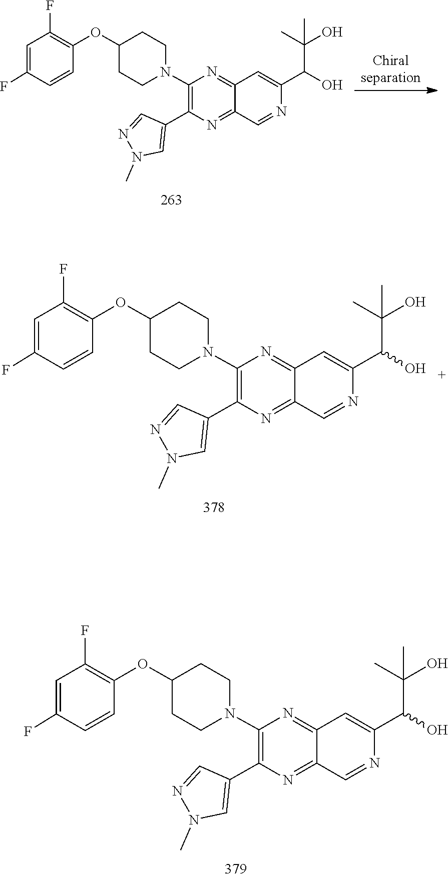 [SMILES: Cn1cc(-c2nc3cnc(C(O)C(C)(C)O)cc3nc2N2CCC(Oc3ccc(F)cc3F)CC2)cn1 ; Cn1cc(-c2nc3cnc(C(O)C(C)(C)O)cc3nc2N2CCC(Oc3ccc(F)cc3F)CC2)cn1 ; Cn1cc(-c2nc3cnc(C(O)C(C)(C)O)cc3nc2N2CCC(Oc3ccc(F)cc3F)CC2)cn1]

Compound 263 (60.0 mg, 0.118 mmol) was submitted for chromatographic chiral separation using a Waters SFC Prep 150 system under the conditions described below:

| | |
|---|---|
| Column | Phenomenex |
| Manufacturer | |
| Column Description | Lux ® 5 μm Cellulose-2, LC Column 250 × 10 |
| | mm, Particle size 5 μm, Pore size 1000 Å |
| Stationary phase | Cellulose tris(3-chloro-4-methylphenylcarbamate) |
| Mode | Isocratic |
| Mobile phase | 50% MeOH + 10 mM Ammonium formate |
| | 50% supercritical CO₂ |
| Flow rate | 10 mL/min |
| Backpressure | 150 bar |
| Column Temperature | 40° C. |
| Run time (min) | 8 |
| Retention time (min) | Isomer 1 = Compound 378 = 5.84 min |
| | Isomer 2 = Compound 379 = 7.28 min |

The obtained fractions were evaporated to obtain compounds Isomer 1—Compound 378 (18.5 mg, 31%, 99.8% ee) and Isomer 2—Compound 379 (18.0 mg, 30%, 98.8% ee) as yellow powders.

### Example 43. (Scheme 4) 5-(2-(4-(2,4-difluorophenoxy)piperidin-1-yl)-3-(1-methyl-1H-pyrazol-4-yl)pyrido[3,4-b]pyrazin-7-yl)oxazolidin-2-one (Compound 371) and 5-(2-(4-(2,4-difluorophenoxy)piperidin-1-yl)-3-(1-methyl-1H-pyrazol-4-yl)pyrido[3,4-b]pyrazin-7-yl)-3-methyloxazolidin-2-one (Compound 482)

### Step f. 5-(2-(4-(2,4-difluorophenoxy)piperidin-1-yl)-3-(1-methyl-1H-pyrazol-4-yl)pyrido[3,4-b]pyrazin-7-yl)oxazolidin-2-one (Compound 371)

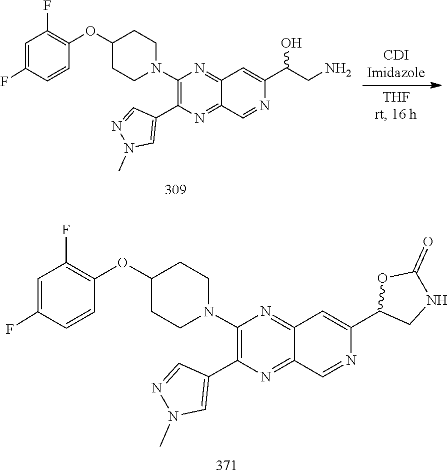 [SMILES: Cn1cc(-c2nc3cnc(C(O)CN)cc3nc2N2CCC(Oc3ccc(F)cc3F)CC2)cn1 ; Cn1cc(-c2nc3cnc(C4CNC(=O)O4)cc3nc2N2CCC(Oc3ccc(F)cc3F)CC2)cn1]

In a 25 mL round bottom flask, Compound 309 (20.0 mg, 41.5 umol) was dissolved in anhydrous THF (0.83 mL). Then, imidazole (2.86 mg, 41.5 umol) and 1,1′-carbonylimidazole (CDI, 8.6 mg, 51.9 umol) were added and the system was stirred at room temperature overnight. The reaction mixture was concentrated under vacuum and the crude was directly purified by reverse phase column chromatography using ammonium formate/ACN as eluents, affording Compound 371 (12.0 mg, 57%) as a yellow solid.

### Step g. 5-(2-(4-(2,4-difluorophenoxy)piperidin-1-yl)-3-(1-methyl-1H-pyrazol-4-yl)pyrido[3,4-b]pyrazin-7-yl)-3-methyloxazolidin-2-one (Compound 482)

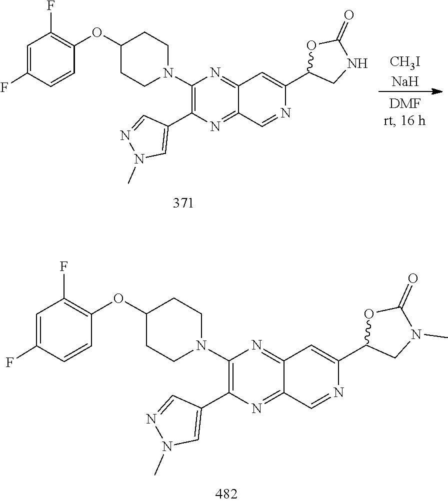 [SMILES: Cn1cc(-c2nc3cnc(C4CNC(=O)O4)cc3nc2N2CCC(Oc3ccc(F)cc3F)CC2)cn1 ; CN1CC(c2cc3nc(N4CCC(Oc5ccc(F)cc5F)CC4)c(-c4cnn(C)c4)nc3cn2)OC1=O]

Compound 482 was synthesized following methylation procedure and was obtained as yellow solid (12.6 mg, 31%).

### Example 44. (Scheme 4) 1-(2-(4-(2,4-difluorophenoxy)piperidin-1-yl)-3-(1-methyl-1H-pyrazol-4-yl)pyrido[3,4-b]pyrazin-7-yl)-2-methoxyethan-1-ol (Compound 437) and 2-(2-(4-(2,4-difluorophenoxy)piperidin-1-yl)-3-(1-methyl-1H-pyrazol-4-yl)pyrido[3,4-b]pyrazin-7-yl)-2-methoxyethan-1-ol (Compound 438)

### Step f. 1-(2-(4-(2,4-difluorophenoxy)piperidin-1-yl)-3-(1-methyl-1H-pyrazol-4-yl)pyrido[3,4-b]pyrazin-7-yl)-2-methoxyethan-1-ol (Compound 437) and 2-(2-(4-(2,4-difluorophenoxy)piperidin-1-yl)-3-(1-methyl-1H-pyrazol-4-yl)pyrido[3,4-b]pyrazin-7-yl)-2-methoxyethan-1-ol (Compound 438)

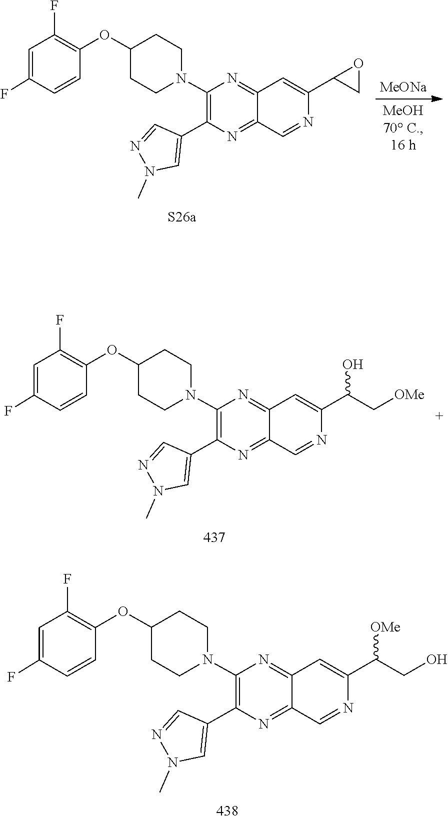 [SMILES: "MeONa" ; Cn1cc(-c2nc3cnc(C4CO4)cc3nc2N2CCC(Oc3ccc(F)cc3F)CC2)cn1 ; COCC(O)c1cc2nc(N3CCC(Oc4ccc(F)cc4F)CC3)c(-c3cnn(C)c3)nc2cn1 ; COC(CO)c1cc2nc(N3CCC(Oc4ccc(F)cc4F)CC3)c(-c3cnn(C)c3)nc2cn1]

In a 25 mL round bottom flask, compound S26a (80.0 mg, 172 umol) was dissolved in anhydrous MeOH (0.86 mL). Then, sodium methoxide (19.6 mg, 344 umol) was slowly added and the system was heated at 70° C. overnight. The reaction mixture was concentrated under vacuum and the crude was directly purified by reverse phase column chromatography using ammonium formate/ACN as eluents, affording Compound 437 (14.5 mg, 17%) and Compound 438 (4.0 mg, 5%) as yellow solids.

### Example 45. (Scheme 4) 3-(2-(4-(2,4-difluorophenoxy)piperidin-1-yl)-3-(1-methyl-1H-pyrazol-4-yl)pyrido[3,4-b]pyrazin-7-yl)-3-hydroxypropanenitrile (Compound 402)

### Step f. 3-(2-(4-(2,4-difluorophenoxy)piperidin-1-yl)-3-(1-methyl-1H-pyrazol-4-yl)pyrido[3,4-b]pyrazin-7-yl)-3-hydroxypropanenitrile (Compound 402)

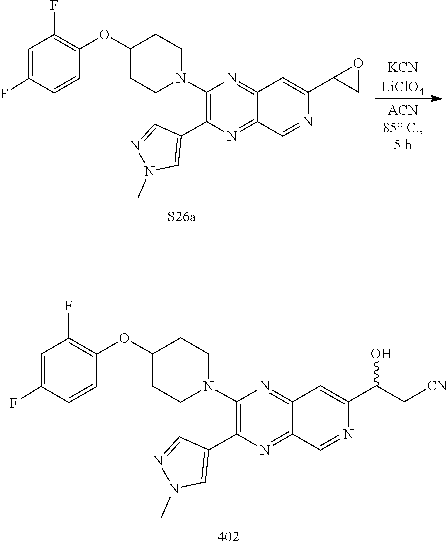

In a 25 mL round bottom flask, compound S26a (95.0 mg, 205 umol) was dissolved in anhydrous ACN (1.0 mL). Then, potassium cyanide (20.4 mg, 307 umol) and lithium perchlorate (24.4 mg, 307 mmol) were slowly added and the system was heated at 85° C. for 5 h. Afterwards, the reaction was quenched with the addition of water (20 mL) and extracted with ethyl acetate (3×). The organic layer was washed with brine, dried over anhydrous MgSO₄, filtered, and concentrated under reduced pressure to give the crude residue. The crude was purified by reverse phase column chromatography using ammonium formate/ACN as eluents, affording Compound 402 (16.2 mg, 16%) as yellow solid.

### Example 46. (Scheme 4) 1-(2-(4-(2,4-difluorophenoxy)piperidin-1-yl)-3-(1-methyl-1H-pyrazol-4-yl)pyrido[3,4-b]pyrazin-7-yl)-2-hydroxy-2-methylpropan-1-one (Compound 413) and 2-(2-(4-(2,4-difluorophenoxy)piperidin-1-yl)-3-(1-methyl-1H-pyrazol-4-yl)pyrido[3,4-b]pyrazin-7-yl)-3-methylbutane-2,3-diol (Compound 434)

### Step f. 1-(2-(4-(2,4-difluorophenoxy)piperidin-1-yl)-3-(1-methyl-1H-pyrazol-4-yl)pyrido[3,4-b]pyrazin-7-yl)-2-hydroxy-2-methylpropan-1-one (Compound 413)

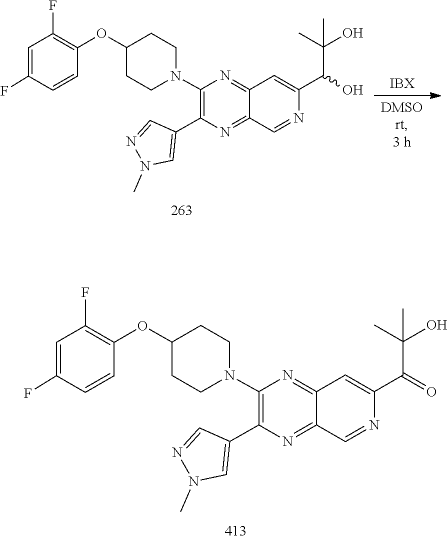

In a reaction vial, Compound 263 (30.0 mg, 0.07 mmol) was dissolved in anhydrous DMSO (1 mL) and the mixture cooled down to 0° C. Then, 2-Iodoxybenzoic acid (IBX, 175.6 mg, 626 umol) was added in three portions every hour at room temperature. Afterwards, reaction was directly injected into a C18 silica column to be purified by reverse phase column chromatography using ammonium formate/ACN as eluents, obtaining Compound 413 as brown powder (25 mg, 63%).

### Step f. 2-(2-(4-(2,4-difluorophenoxy)piperidin-1-yl)-3-(1-methyl-1H-pyrazol-4-yl)pyrido[3,4-b]pyrazin-7-yl)-3-methylbutane-2,3-diol (Compound 434)

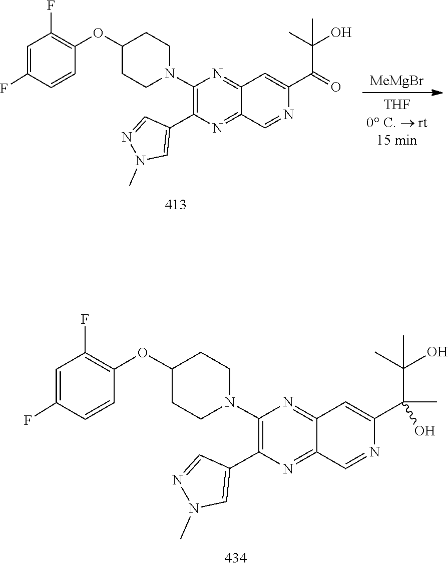 [SMILES: "MeMgBr" ; Cn1cc(-c2nc3cnc(C(=O)C(C)(C)O)cc3nc2N2CCC(Oc3ccc(F)cc3F)CC2)cn1 ; Cn1cc(-c2nc3cnc(C(C)(O)C(C)(C)O)cc3nc2N2CCC(Oc3ccc(F)cc3F)CC2)cn1]

In a reaction vial sealed under nitrogen, Compound 413 (21.0 mg, 41.3 umol) was dissolved in anhydrous THF (1 mL) and the mixture cooled down to 0° C. Then, methylmagnesium bromide solution (3 M in Et₂O, 160 uL, 0.08 mmol) was added slowly and the system stirred for 15 min at room temperature (reaction completion indicated by LCMS analysis). Afterwards, reaction was quenched by the addition of ammonium chloride solution. The system was extracted with Ethyl Acetate (3×). The organic layer was washed with brine and dried over sodium sulfate. Followed by filtration and evaporation of volatiles, the obtained crude was purified by reverse phase column chromatography using ammonium formate/ACN as eluents to obtain Compound 434 as pale-yellow powder (17 mg, 78%).

### Example 47. (Scheme 4) 2-(4-(2,4-difluorophenoxy)piperidin-1-yl)-N-methyl-3-(1-methyl-1H-pyrazol-4-yl)pyrido[3,4-b]pyrazin-7-amine (Compound 433)

### Step g. 7-allyl-2-(4-(2,4-difluorophenoxy)piperidin-1-yl)-3-(1-methyl-1H-pyrazol-4-yl)pyrido[3,4-b]pyrazine (S24d)

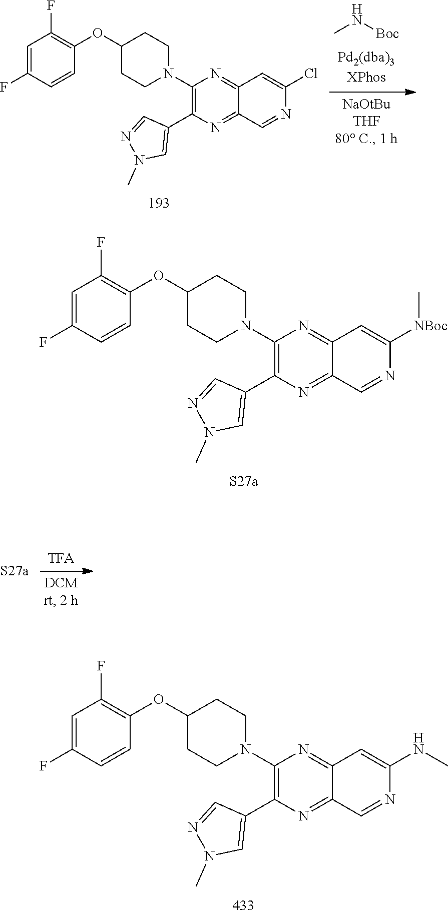

Step 1. In a microwave vial, Compound 193 (50.0 mg, 109 umol), tert-butyl-N-methylcarbamate (14.8 mg, 109 umol), 2-dicyclohexylphosphino-2′,4′,6′-triisopropylbiphenyl (XPhos, 21.5 mg, 43.8 umol), sodium tert-butoxide (21.1 mg, 219 umol) and tris(dibenzylideneacetone)-dipalladium(0) (20.0 mg, 21.9 umol) were dissolved in anhydrous THF (2.0 mL). The obtained mixture was degassed for 15 minutes using nitrogen gas bubbling, then, the system was sealed and heated at 80° C. for 1 h. Afterwards, the mixture was diluted with EtOAc, filtered through celite/silica layer, and the filtrate concentrated under reduced pressure to be used in the next step.

Step 2. The resulting crude was diluted with DCM (2.00 mL) and trifluoroacetic acid (0.63 mL, 8.22 mmol) was added dropwise at room temperature. Then, reaction was stirred for 2 h. Afterwards, the solvents were evaporated under vacuum and the obtained crude directly purified by reverse phase column chromatography using ammonium bicarbonate/ACN as eluents to obtain Compound 433 as yellow fluorescent powder (23.6 mg, 48%).

### Example 48. (Scheme 4) 2-(4-(2,4-difluorophenoxy)piperidin-1-yl)-7-methoxy-3-(1-methyl-1H-pyrazol-4-yl)pyrido[3,4-b]pyrazine (Compound 410)

### Step g. 2-(4-(2,4-difluorophenoxy)piperidin-1-yl)-7-methoxy-3-(1-methyl-1H-pyrazol-4-yl)pyrido[3,4-b]pyrazine (Compound 410)

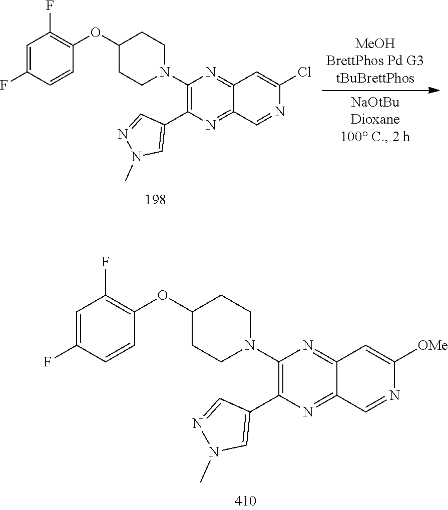 [SMILES: Cn1cc(-c2nc3cnc(Cl)cc3nc2N2CCC(Oc3ccc(F)cc3F)CC2)cn1 ; COc1cc2nc(N3CCC(Oc4ccc(F)cc4F)CC3)c(-c3cnn(C)c3)nc2cn1]

Step 1. In a microwave vial, Compound 198 (50.0 mg, 109 umol), methanol (44.8 uL, 1.09 mmol), 2-(di-t-butylphosphino)-3,6-dimethoxy-2′,4′,6′-tri-i-propyl-1,1′-biphenyl (tBuBrettPhos, 2.73 mg, 5.47 umol), sodium tert-butoxide (15.8 mg, 164 umol) and BrettPhos Pd G3 (5.06 mg, 5.47 umol) were dissolved in anhydrous dioxane (1.67 mL). The obtained mixture was degassed for 15 minutes using nitrogen gas bubbling, then, the system was sealed and heated at 100° C. for 2 h. Afterwards, the mixture was diluted with EtOAc, filtered through celite/silica layer, and the filtrate concentrated under reduced pressure to be directly purified by reverse phase column chromatography using ammonium bicarbonate/ACN as eluents to obtain Compound 410 as off-white powder (10.1 mg, 20%).

### Example 49. (Scheme 2) 2-(4-(2,4-difluorophenoxy)piperidin-1-yl)-3-(1-methyl-1H-pyrazol-4-yl)pyrido[3,4-b]pyrazin-7-ol (Compound 395)

### Step g. 2-(4-(2,4-difluorophenoxy)piperidin-1-yl)-3-(1-methyl-1H-pyrazol-4-yl)pyrido[3,4-b]pyrazin-7-ol (Compound 395)

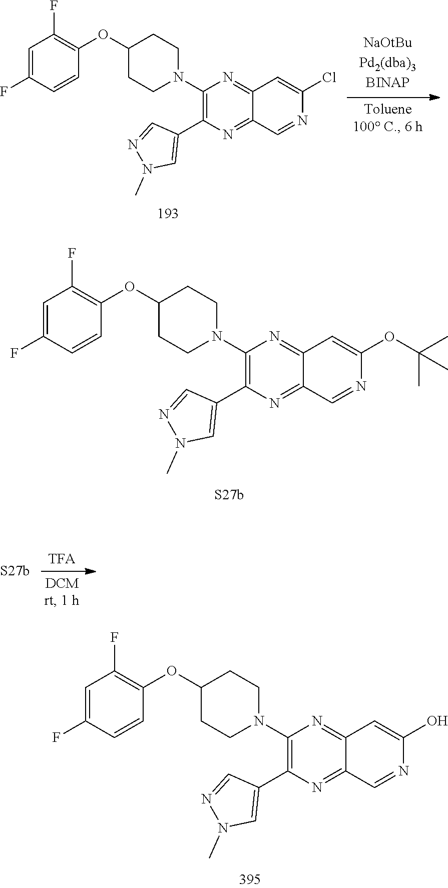 [SMILES: Cn1cc(-c2nc3cnc(Cl)cc3nc2N2CCC(Oc3ccc(F)cc3F)CC2)cn1 ; Cn1cc(-c2nc3cnc(OC(C)(C)C)cc3nc2N2CCC(Oc3ccc(F)cc3F)CC2)cn1 ; Cn1cc(-c2nc3cnc(O)cc3nc2N2CCC(Oc3ccc(F)cc3F)CC2)cn1]

Step 1. In a microwave vial, Compound 193 (200 mg, 438 umol), 2,2-bis(diphenylphosphino)-1,1-binaphthyl (BINAP, 27.8 mg, 43.8 umol), sodium tert-butoxide (211 mg, 2.19 mmol) and tris(dibenzylideneacetone)-dipalladium(0) (20.0 mg, 21.9 umol) were dissolved in toluene (7.0 mL). The obtained mixture was degassed for 15 minutes using nitrogen gas bubbling, then, the system was sealed and heated at 100° C. for 6 h. Afterwards, the mixture was diluted with EtOAc, filtered through celite/silica layer, and the filtrate concentrated under reduced pressure to be used in the next step.

Step 2. In a round bottom flask, trifluoroacetic acid (1.94 mL, 25.1 mmol) was added to a solution of crude S27b. Then, the mixture was stirred at room temperature for 1 h. Afterwards, the solvents were evaporated under vacuum and the obtained crude directly purified by reverse phase column chromatography using ammonium formate/ACN as eluents to obtain Compound 395 as yellow powder (50.0 mg, 26%).

### Example 50. (Scheme 4) 3-(2-(4-(2,4-difluorophenoxy)piperidin-1-yl)-3-(1-methyl-1H-pyrazol-4-yl)pyrido[3,4-b]pyrazin-7-yl)propane-1,2-diol (Compound 377)

### Step d. 7-allyl-2-(4-(2,4-difluorophenoxy)piperidin-1-yl)-3-(1-methyl-H-pyrazol-4-yl)pyrido[3,4-b]pyrazine (S24d)

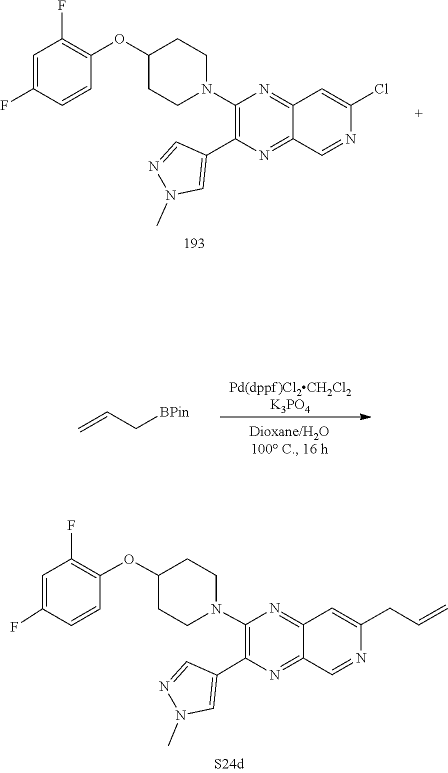

Compound S24d was synthesized following procedure using Pd(dppf)Cl₂·CH₂Cl₂ as catalyst and was obtained as yellow oil (323 mg, 64%).

### Step e. 3-(2-(4-(2,4-difluorophenoxy)piperidin-1-yl)-3-(1-methyl-1H-pyrazol-4-yl)pyrido[3,4-b]pyrazin-7-yl)propane-1,2-diol (Compound 377)

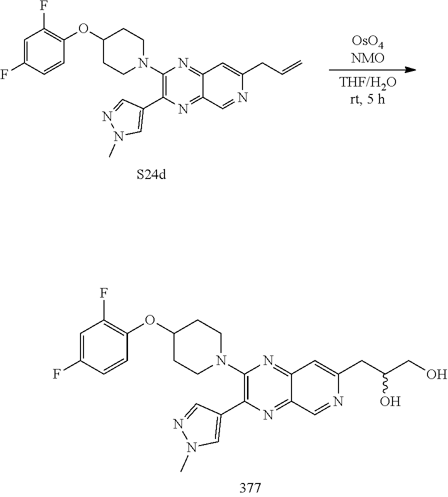 [SMILES: C=CCc1cc2nc(N3CCC(Oc4ccc(F)cc4F)CC3)c(-c3cnn(C)c3)nc2cn1 ; Cn1cc(-c2nc3cnc(CC(O)CO)cc3nc2N2CCC(Oc3ccc(F)cc3F)CC2)cn1]

Compound 377 was synthesized following procedure and was obtained as yellow powder (13.4 mg, 39%).

### Example 51. (Scheme 4) 1-(2-(4-(2,4-difluorophenoxy)piperidin-1-yl)-3-(1-methyl-1H-pyrazol-4-yl)pyrido[3,4-b]pyrazin-7-yl)-2-methylpropan-2-ol (Compound 461)

### Step f. 2-(4-(2,4-difluorophenoxy)piperidin-1-yl)-7-(3,3-dimethyloxiran-2-yl)-3-(1-methyl-1H-pyrazol-4-yl)pyrido[3,4-b]pyrazine (S26b)

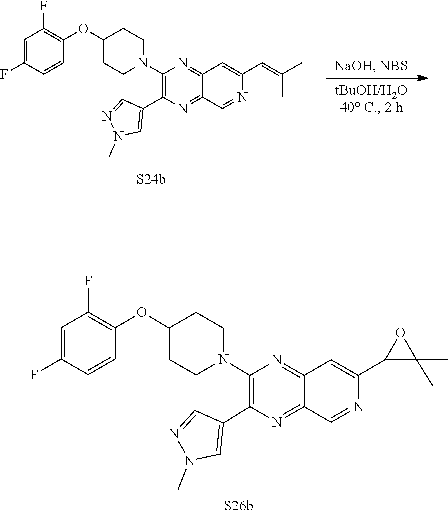 [SMILES: CC(C)=Cc1cc2nc(N3CCC(Oc4ccc(F)cc4F)CC3)c(-c3cnn(C)c3)nc2cn1 ; Cn1cc(-c2nc3cnc(C4OC4(C)C)cc3nc2N2CCC(Oc3ccc(F)cc3F)CC2)cn1]

Compound S26b was synthesized following epoxidation and was obtained as brown powder (136 mg, 72%).

### Step f. 1-(2-(4-(2,4-difluorophenoxy)piperidin-1-yl)-3-(1-methyl-1H-pyrazol-4-yl)pyrido[3,4-b]pyrazin-7-yl)-2-methylpropan-2-ol (Compound 461)

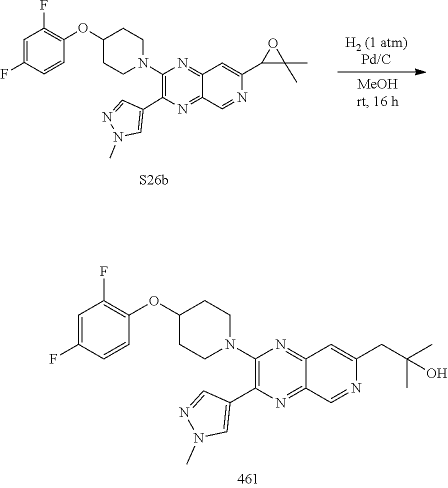 [SMILES: Cn1cc(-c2nc3cnc(C4OC4(C)C)cc3nc2N2CCC(Oc3ccc(F)cc3F)CC2)cn1 ; Cn1cc(-c2nc3cnc(CC(C)(C)O)cc3nc2N2CCC(Oc3ccc(F)cc3F)CC2)cn1]

In a 25 mL round bottom flask, compound S26b (135 mg, 274 umol) was dissolved in MeOH (5.56 mL) and wet palladium on carbon (10% wt, 5.8 mg, 54.8 umol) was added. The system was sealed with a rubber septa and hydrogen gas was bubbled for 10 min, then, the system was stirred overnight under hydrogen atmosphere at room temperature. Afterwards, the reaction mixture was filtered through Celite using ethyl acetate. The filtrate was concentrated under vacuum and the crude was directly purified by reverse phase column chromatography using ammonium formate/ACN as eluents, affording Compound 461 (15.0 mg, 11%) as pale-yellow solid.

### Example 52. (Scheme 4) 6-(2-(4-(2,4-difluorophenoxy)piperidin-1-yl)-3-(1-methyl-1H-pyrazol-4-yl)pyrido[3,4-b]pyrazin-7-yl)morpholin-3-one (Compound 464)

### Step f. 2-chloro-N-(2-(2-(4-(2,4-difluorophenoxy)piperidin-1-yl)-3-(1-methyl-1H-pyrazol-4-yl)pyrido[3,4-b]pyrazin-7-yl)-2-hydroxyethyl)acetamide (S27c)

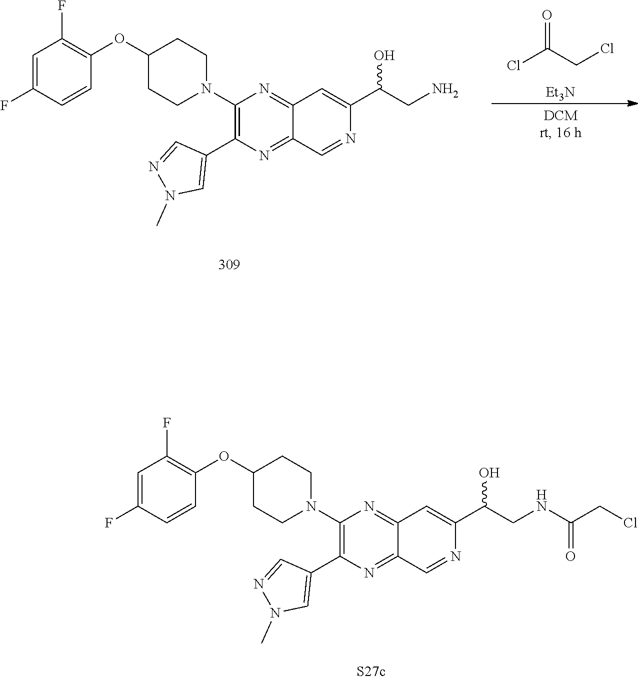 [SMILES: "Et3N" ; Cn1cc(-c2nc3cnc(C(O)CN)cc3nc2N2CCC(Oc3ccc(F)cc3F)CC2)cn1 ; O=C(Cl)CCl ; Cn1cc(-c2nc3cnc(C(O)CNC(=O)CCl)cc3nc2N2CCC(Oc3ccc(F)cc3F)CC2)cn1]

In a 25 mL round bottom flask, Compound 309 (170 mg, 353 umol) was dissolved in anhydrous DCM (5.04 mL) and cooled to 0° C. Then, triethylamine (148 uL, 1.06 mmol) and chloroacetyl chloride (31.2 uL, 388 umol) were sequentially added and the system was stirred at room temperature overnight). Afterwards, reaction was quenched by the addition of ammonium chloride solution. The system was extracted with DCM (3×). The organic layer was washed with brine and dried over sodium sulfate. Followed by filtration and evaporation of volatiles, the obtained crude was purified by column chromatography using DCM/Methanol as eluents, affording S27c (72.0 mg, 37%) as a brown solid.

### Step f. 6-(2-(4-(2,4-difluorophenoxy)piperidin-1-yl)-3-(1-methyl-1H-pyrazol-4-yl)pyrido[3,4-b]pyrazin-7-yl)morpholin-3-one (Compound 464)

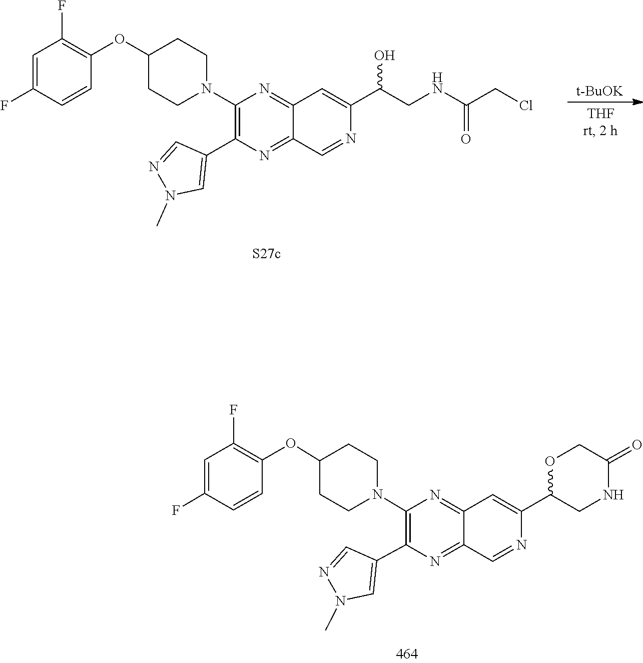

In a 25 mL round bottom flask, compound S27c (54.0 mg, 96.8 umol) and potassium tert-butoxide (12.2 mg, 106 umol) were dissolved in anhydrous THF (3.87 mL) and the system was stirred at room temperature for 2 h. Afterwards, reaction was quenched by the addition of ammonium chloride solution. The system was extracted with ethyl acetate (3×). The organic layer was washed with brine and dried over sodium sulfate. Followed by filtration and evaporation of volatiles, the obtained crude was purified by reverse phase column chromatography using ammonium formate/ACN as eluents, affording Compound 464 (19.7 mg, 39%) as yellow solid.

### Example 53. (Scheme 4) 1-(2-(4-(2,4-difluorophenoxy)piperidin-1-yl)-3-(1-methyl-1H-pyrazol-4-yl)pyrido[3,4-b]pyrazin-7-yl)ethane-1,2-diol (Compound 470)

### Step f. 2-amino-1-(2-(4-(2,4-difluorophenoxy)piperidin-1-yl)-3-(1-methyl-1H-pyrazol-4-yl)pyrido[3,4-b]pyrazin-7-yl)ethan-1-ol (Compound 470)

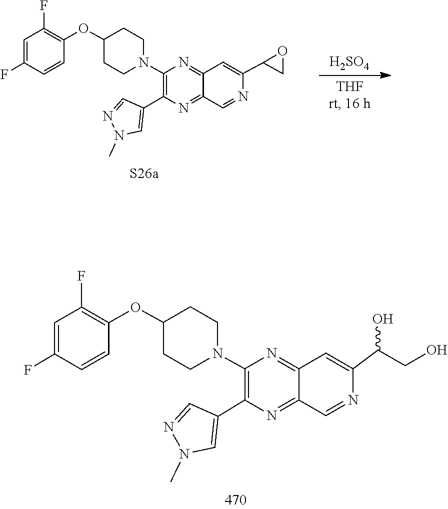

In a 25 mL round bottom flask, compound S26a (59.0 mg, 0.107 mmol) was dissolved in THF (5.00 mL). Then, aqueous solution of sulfuric acid (1 M, 5 mL) was added and the system was stirred at room temperature overnight. Afterwards, saturated aqueous solution of sodium carbonate was added to adjust pH to 7. The system was extracted with DCM (3×). The organic layer was washed with brine and dried over sodium sulfate. Followed by filtration and evaporation of volatiles, the obtained crude was purified by reverse phase column chromatography using ammonium formate/ACN as eluents, affording Compound 470 (5.26 mg, 10%) as yellow solid.

### Example 54. (Scheme 4) 2-(4-(2,4-difluorophenoxy)piperidin-1-yl)-3-(1-methyl-1H-pyrazol-4-yl)-9a,10-dihydro-5H-oxazolo[3′,4′:1,6]pyrido[3,4-b]pyrazin-7(9H)-one (Compound 486)

### Step e. 2-(4-(2,4-difluorophenoxy)piperidin-1-yl)-3-(1-methyl-1H-pyrazol-4-yl)pyrido[3,4-b]pyrazine-7-carbaldehyde (Compound 263)

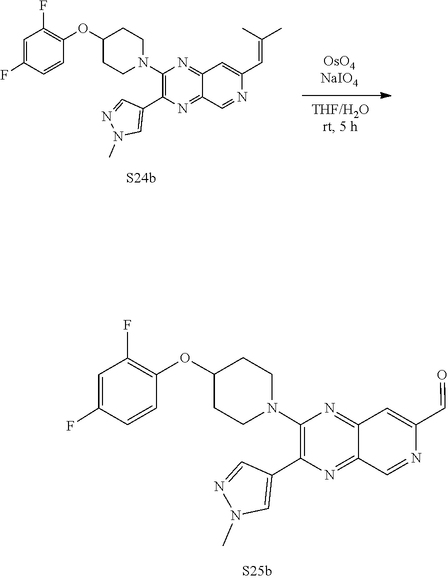 [SMILES: CC(C)=Cc1cc2nc(N3CCC(Oc4ccc(F)cc4F)CC3)c(-c3cnn(C)c3)nc2cn1 ; Cn1cc(-c2nc3cnc(C=O)cc3nc2N2CCC(Oc3ccc(F)cc3F)CC2)cn1]

In a 25 mL round bottom flask, compound S26a (59.0 mg, 0.107 mmol) and sodium periodate (909 mg, 4.24 mmol) were dissolved in a mixture of THF (17.7 mL) and H₂O (3.53 mL). Then, the mixture was treated with 4% in water solution of osmium tetroxide (2.69 mL, 0.424 mmol). The system was stirred at room temperature for 5 h. Afterwards, The reaction mixture was filtered through silica pad using 20% MeOH in DCM. The filtrate was concentrated and the obtained crude was purified by column chromatography using DCM/ethyl acetate as eluents, affording S25b (500 mg, 52%) as yellow solid.

### Step e. (2-(4-(2,4-difluorophenoxy)piperidin-1-yl)-3-(1-methyl-1H-pyrazol-4-yl)pyrido[3,4-b]pyrazin-7-yl)methanol (S26c)

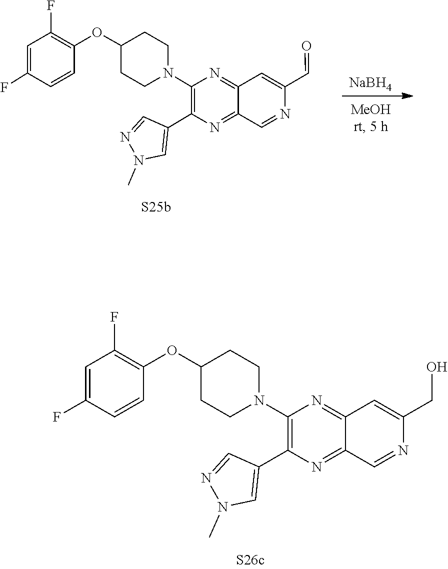 [SMILES: Cn1cc(-c2nc3cnc(C=O)cc3nc2N2CCC(Oc3ccc(F)cc3F)CC2)cn1 ; "NaBH4" ; Cn1cc(-c2nc3cnc(CO)cc3nc2N2CCC(Oc3ccc(F)cc3F)CC2)cn1]

Compound S26c was synthesized following reduction procedure and was obtained as yellow powder (275 mg, 60%).

### Step e. (6-benzyl-2-(4-(2,4-difluorophenoxy)piperidin-1-yl)-3-(1-methyl-1H-pyrazol-4-yl)-5,6,7,8-tetrahydropyrido[3,4-b]pyrazin-7-yl)methanol (S26d)

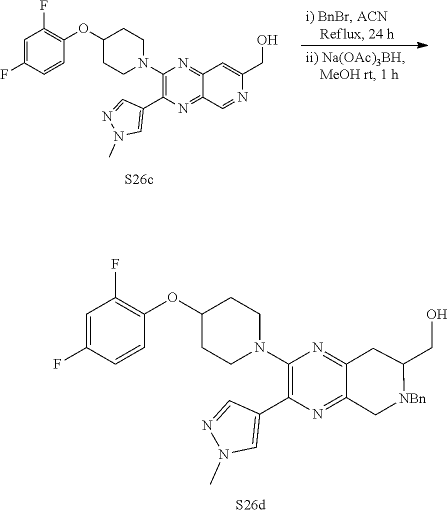 [SMILES: Cn1cc(-c2nc3cnc(CO)cc3nc2N2CCC(Oc3ccc(F)cc3F)CC2)cn1 ; Cn1cc(-c2nc3c(nc2N2CCC(Oc4ccc(F)cc4F)CC2)CC(CO)N(Cc2ccccc2)C3)cn1]

Compound S26d was synthesized following benzylation/reduction sequence procedure and was obtained as orange oil (254 mg, 77%).

### Step g. (2-(4-(2,4-difluorophenoxy)piperidin-1-yl)-3-(1-methyl-1H-pyrazol-4-yl)-5,6,7,8-tetrahydropyrido[3,4-b]pyrazin-7-yl)methanol (S26e)

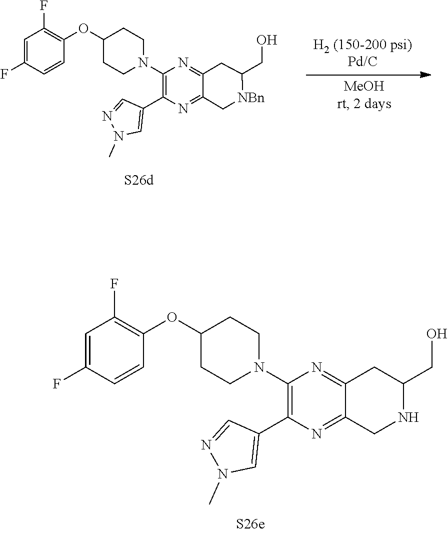 [SMILES: Cn1cc(-c2nc3c(nc2N2CCC(Oc4ccc(F)cc4F)CC2)CC(CO)N(Cc2ccccc2)C3)cn1 ; Cn1cc(-c2nc3c(nc2N2CCC(Oc4ccc(F)cc4F)CC2)CC(CO)NC3)cn1]

Compound S26e was synthesized following hydrogenation procedure and was obtained as yellow powder (92 mg, 53%).

### Step g. 2-(4-(2,4-difluorophenoxy)piperidin-1-yl)-3-(1-methyl-1H-pyrazol-4-yl)-9a,10-dihydro-5H-oxazolo[3′,4′:1,6]pyrido[3,4-b]pyrazin-7(9H)-one (Compound 486)

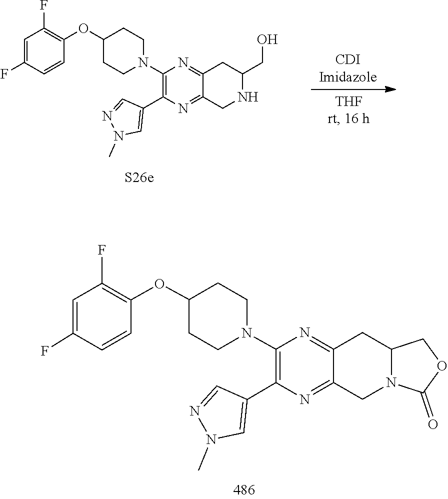 [SMILES: Cn1cc(-c2nc3c(nc2N2CCC(Oc4ccc(F)cc4F)CC2)CC(CO)NC3)cn1 ; Cn1cc(-c2nc3c(nc2N2CCC(Oc4ccc(F)cc4F)CC2)CC2COC(=O)N2C3)cn1]

Compound 486 was synthesized following cyclization procedure and was obtained as white solid (6.09 mg, 41%).

### Example 55. (Scheme 6) (Rel-R)-1-(3-(4-(2,4-difluorobenzyl)piperazin-1-yl)-2-(1-methyl-1H-pyrazol-4-yl)pyrido[3,4-b]pyrazin-7-yl)-2-methylpropane-1,2-diol (Compound 369) and (Rel-S)-1-(3-(4-(2,4-difluorobenzyl)piperazin-1-yl)-2-(1-methyl-1H-pyrazol-4-yl)pyrido[3,4-b]pyrazin-7-yl)-2-methylpropane-1,2-diol (Compound 370)

### Step h. (Rel-R)-1-(3-(4-(2,4-difluorobenzyl)piperazin-1-yl)-2-(1-methyl-1H-pyrazol-4-yl)pyrido[3,4-b]pyrazin-7-yl)-2-methylpropane-1,2-diol (Compound 369) and (Rel-S)-1-(3-(4-(2,4-difluorobenzyl)piperazin-1-yl)-2-(1-methyl-H-pyrazol-4-yl)pyrido[3,4-b]pyrazin-7-yl)-2-methylpropane-1,2-diol (Compound 370)

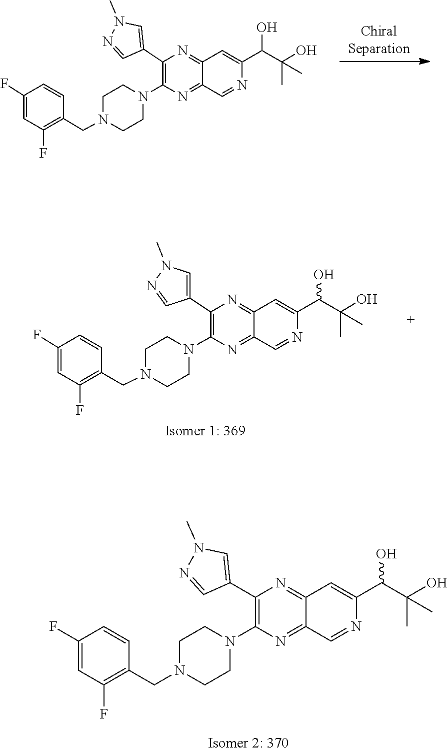 [SMILES: Cn1cc(-c2nc3cc(C(O)C(C)(C)O)ncc3nc2N2CCN(Cc3ccc(F)cc3F)CC2)cn1 ; Cn1cc(-c2nc3cc(C(O)C(C)(C)O)ncc3nc2N2CCN(Cc3ccc(F)cc3F)CC2)cn1 ; Cn1cc(-c2nc3cc(C(O)C(C)(C)O)ncc3nc2N2CCN(Cc3ccc(F)cc3F)CC2)cn1]

Compound 268 (51.0 mg, 0.100 mmol) was submitted for chromatographic chiral separation using a Waters SFC Prep 150 system under the conditions described below:

| | |
|---|---|
| Column | Chiral Technologies Inc. |
| Manufacturer | |
| Column Description | ChiralPak IG, LC Column 250 × 10 mm, Particle |
| | size 5 μm, Pore size 1000 Å |
| Stationary phase | Amylose tris(3-chloro-5-methylphenylcarbamate) |
| Mode | Isocratic |
| Mobile phase | 50% MeOH + 10 mM Ammonium formate |
| | 50% supercritical CO₂ |
| Flow rate | 10 mL/min |
| Backpressure | 150 bar |
| Column Temperature | 40° C. |
| Run time (min) | 20 |
| Retention time (min) | Isomer 1 - Compound 369 = 13.44 min |
| | Isomer 2 - Compound 370 = 16.93 min |

The obtained fractions were evaporated to obtain compounds Isomer 1—Compound 369 (14.7 mg, 29%, 98.3% ee) and Isomer 2—Compound 370 (14.3 mg, 28%, 99.9% ee) as yellow powders.

### Example 56. GPR6 and GPR3 Antagonist Functional cAMP Assay

CHO cells stably expressing GPR6 or GPR3 receptors were dissociated from cell culture flasks and incubated for 2 h at 37° C. in a 10 cm dish in a humidified incubator. Cells were dissociated, counted to have 1e6 cells/mL (4.5e6 cells needed/384-well plate), washed once in starved media, than in D-PBS and finally suspended in assay buffer (Krebs-Ringer Bicarbonate Buffer with 1800 mg/L glucose, without calcium chloride and sodium bicarbonate (Sigma) in the presence of 0.1% BSA and 150 μM of Ro 20-1724 Phosphodiesterase inhibitor).

Cells in suspension from above were plated in 384-well white Optiplate (PerkinElmer) at 10,000 cells/well/10 μl, briefly centrifuged prior direct addition of compounds (50 nL/well) in 10 mM DMSO using a Tecan D300e Digital Dispenser, final DMSO in each well was normalized to 0.1%. Cells were incubated in the presence of compounds for 45 min at 37° C. in a humidified incubator.

To measure cAMP levels, LANCE® Ultra cAMP assay (Perkin Elmer), a homogeneous time-resolved fluorescence resonance energy transfer (TR-FRET) immunoassay was used following manufacturer's recommendations. Plates were shaken for 1 h at room temperature before reading on a Tecan InfiniteF200Pro multi-mode plate reader using standard FRET settings (Excitation 340 nm, Emission at 615 nm). FRET values were reported as readings obtained at 665 nm. Data were normalized to DMSO (0.1%) and reference compounds (100%) and fit to a 4-parameter logistical fit to generate antagonist IC50s.

### Example 57. hERG Assay

Stably-transfected HEK293/hERG cells were seeded on poly-ornithine coated glass coverslip and incubated 3-24 h in 35 mm dishes before transferring to the recording chamber. The recording chamber on the stage of a Nikon inverted microscope is continuously perfused with extracellular solution (1-1.5 ml/min). Electrodes were pulled, polished and filled with intracellular solution (tip resistance ˜3-5MΩ). After establishing a tight seal (resistances 1GΩ) between pipette tip and cell membrane, a brief suction is applied to rupture the patched membrane to establish the whole-cell patch clamp configuration. Cells are voltage clamped at −80 mV. The cell was first depolarized to +40 mV for 2s to activate hERG current, and then clamped to −40 mV for 3s to record a tail current. Test articles in various concentrations controlled by a voltage command 8 pinch valve superfusion system were delivered to the proximity of the test cell to record hERG current changes under different conditions with recording done within 30 min.

Intracellular solution (in mM): 130 KCl, 1.0 MgCl₂, 5.0 EGTA, 10 HEPES, 5.0 Mg-ATP; adjust pH to 7.25 with 1M KOH; osmolarity approximately at 280 mOsm. Extracellular solution (in mM): 137 NaCl, 1.8 CaCl₂, 1.0 MgCl2, 4.0 KCl, 10 Glucose, 10 HEPES; adjust pH to 7.4 with 1 M NaOH; osmolarity approximately at 295 mOsm. Original compound was dissolved in 100% DMSO, then serial diluted with DMSO to respective 1000× of test concentration to make DMSO sub-stocks. The DMSO sub-stocks were diluted 1000 fold to testing solutions (0.1, 0.3, 1, 3, 10, and 30 μM) using extracellular solution. Rapid exposure to test compound was achieved by utilizing a multichannel solenoid-operated flow system. Testing compounds were sequentially applied in increasing order of concentrations after initial application of vehicle solution on EPC-10 manual patch clamping device with the following:
Amplifier: HEKA EPC-10 USB
Nikon Ti-S Inverted Microscope
MP-285/R micromanipulator system
DAD-8VCPP voltage command Pinch valve superfusion system
TMC Shock Absorption Platform
Peristaltic Pump
Sutter Puller
Vapor pressure osmometer
Temperature controller

hERG data of selected compounds are listed in the table below.

| Compound | hERG Assay GMean IC50 (nM) |
|---|---|
| 707 | >30000 |
| 716 | 24300 |
| 575 | 21710 |
| 577 | 13630 |

### Example 58. Specific Optical Rotation (SOR) Method of Analysis (MoA)

Reagents: Methanol
Equipment: SOR, Jasco P2000

### Instrument Parameters:

Light source: WI (Tungsten-halogen lamp)
Wavelength: 589 nm
Path length: 10 mm
Temperature: 25° C.
Number of Scans: 5

### Sample PREPARATION:

Weigh 5 mg of the sample and dissolved it in 1 mL of diluent (methanol).

### Procedure:

Measure the specific optical rotation of the sample in a Polarimeter at 25±0.5° C.

Conduct the blank with diluent, feed the rotation of the blank and sample concentration on Polarimeter. Calculate the specific optical rotation of the sample on as is basis using the formula given below. The average result from the five scans will be considered for the calculation of SOR.

$$Specific optical rotation = \frac{Optical rotation}{Path length \times Concentration of sample}$$

The data are included in table 1.

### Example 59. Synthesis of Compound 593

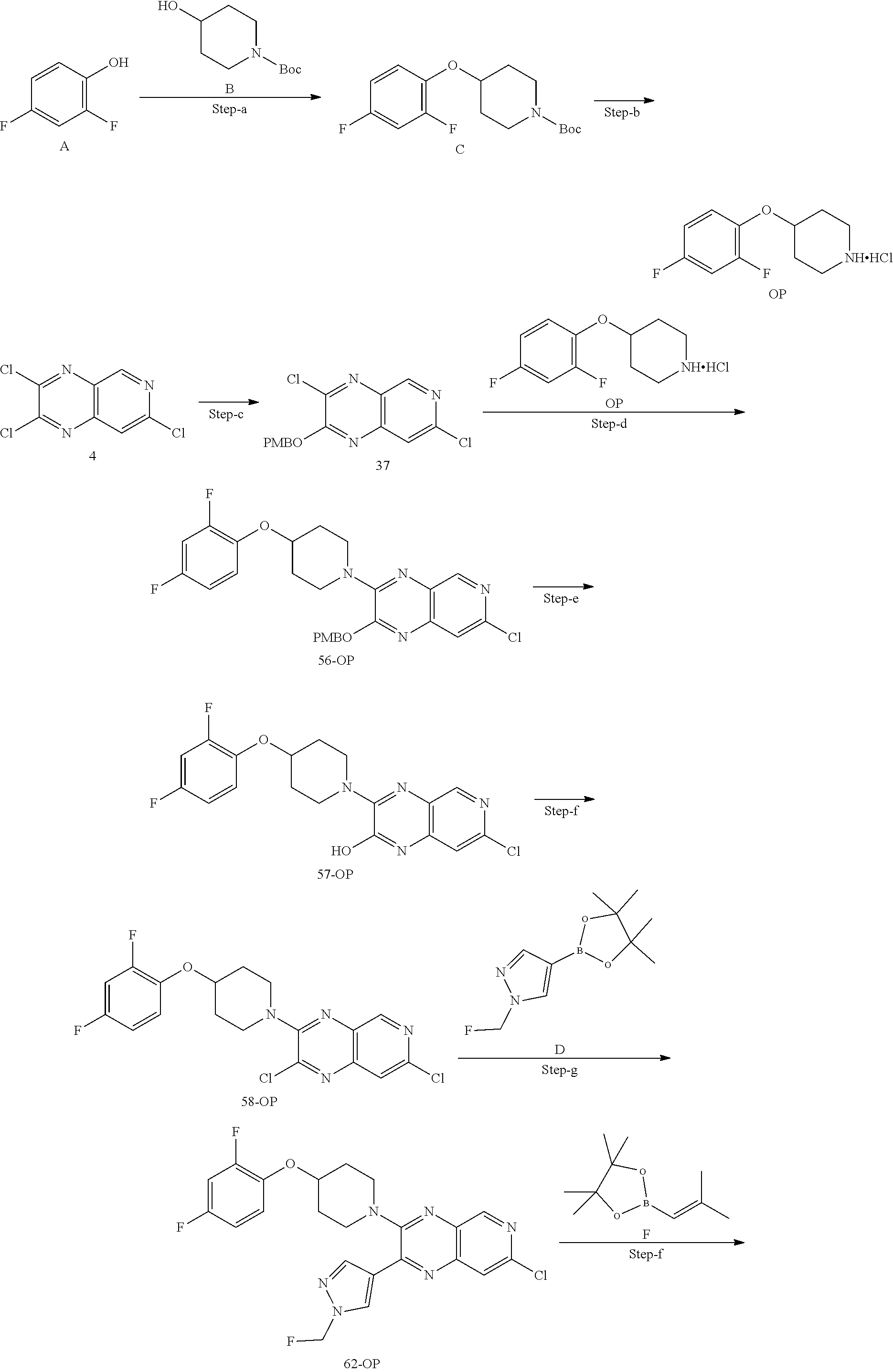

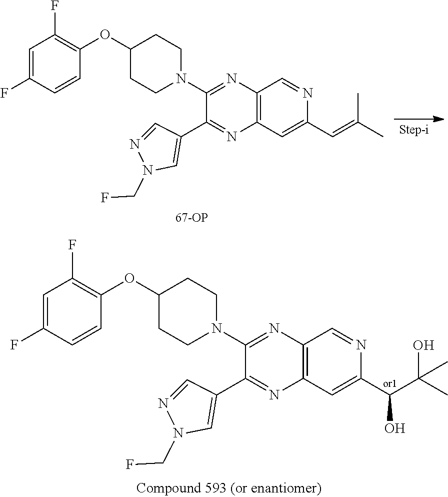 [SMILES: CC(C)=Cc1cc2nc(-c3cnn(CF)c3)c(N3CCC(Oc4ccc(F)cc4F)CC3)nc2cn1 ; CC(C)(O)[C@@H](O)c1cc2nc(-c3cnn(CF)c3)c(N3CCC(Oc4ccc(F)cc4F)CC3)nc2cn1]

General Scheme for synthesis of compound 593: Conditions: a) DIAD, PPh₃, Toluene, 60° C., 16h; b). 4M HCl in dioxane, rt, 2h. c) PMB-OH, K₂CO₃, ACN, 40° C., 4h; d). OP, K₂CO₃, KF, ACN, rt, 4h; e) 4M HCl in dioxane, rt, 4h; f). SOCl₂, 80° C., 16h, g). Comp-D, Pd(dppfCl₂). DCM, K₂CO₃, Dioxane, 100° C., 16h, sealed tube; h) Comp-F, Pd(dppfCl₂). DCM, K₂CO₃, Dioxane/water (4:1), 100° C., 4h, sealed tube; i) Beta-ADmix, H₂NSO₂Me, t-BuOH/H₂O (1:1), 0° C. to rt, 18 h; Chiral separation.

### Step-a: tert-buty 4-(2,4-difluorophenoxy) piperidine-1-carboxylate (C)

To the stirred solution of comp-B (100 g, 497 mmol, 1 eq) and Comp-A (64 g, 497 mmol, 1 eq) in Toluene (1 L, 10 Vol) at 0° C. was added PPh₃ (260 g, 994 mmol, 2 eq) followed by DIAD (200 mL, 995 mmol, 2 eq) dropwise over 15-20 min at 0-5° C. The reaction mass temperature was raised to 60° C. and stirred for 16 h. The progress of the reaction was monitored by TLC. After completion of the reaction, it was cooled to room temperature and evaporated under vacuum to get brown gum. The resulting gummy material was triturated with Hexane (1 L), filtered the solid to remove TPP Oxide. The solid was washed with Hexane (500 mL). Combined filtrates were concentrated under reduced pressure to obtain Comp-C (200 g, quantitative yield) as brown syrup. The crude compound was used in next step without purification.

### Step-b: 4-(2,4-difluorophenoxy)piperidine Hydrochloride (OP)

To the neat comp-C (100 g) added 4M HCl in Dioxane (1 L, 10 Vol) at room temperature. The resulting suspension was stirred for 1-2 h at 25-30° C. The progress of the reaction was monitored by TLC. After completion of the reaction, it was evaporated under vacuum to remove the solvent. The resulting solid taken into Acetonitrile (500 mL, 5 Vol), stirred for 15-20 min at rt. The slurry was filtered to get compound-OP (72 g) as an off white solid. The solid was dried under high vacuum for 10-15 min at room temperature to afford Comp-OP (HCl salt) (69 g, 55% (After 2 steps yield) as an off-white solid.

### Step-c: 3,7-dichloro-2-((4-methoxybenzyl)oxy)pyrido[3,4-b]pyrazine (37)

To the stirred suspension of comp-4 (40 g, 172 mmol, 1 eq) and PMB-OH (21 mL, 172 mmol, 1 eq) in ACN (400 mL) was added K₂CO₃ (71 g, 516 mmol, 3 eq) at room temperature. The reaction mass temperature was raised to 40-50° C. and stirred for 4-6h. The progress of the reaction was monitored by TLC. After completion of the reaction, it was cooled to room temperature, poured into ice cold water (1 L) and stirred for 10-15 min. Filtered the resultant solid to obtain crude compound. The crude compound was triturated with MTBE/Pentane (1:1) (400 mL) to obtain Comp-37 (30 g, 52%) as brown solid.

### Step-d: 7-chloro-3-(4-(2,4-difluorophenoxy)piperidin-1-yl)-2-((4-methoxybenzyl)oxy)pyrido [3,4-b]pyrazine (56-OP)

To the stirred solution of comp-37 (30 g, 89 mmol, 1 eq) in Acetonitrile (300 mL) was added K₂CO₃ (36.9 g, 267 mmol, 3 eq), KF (5.1 g, 89 mmol, 1 eq), followed by OP (22.7 g, 106.8 mmol, 1.2 eq) at 0-5° C. The reaction mixture temperature was raised to 25-30° C. and stirred for 4-6h. The progress of the reaction was monitored by TLC. After completion of the reaction, it was cooled to room temperature, poured into ice cold water (1 L) and stirred for 10-15 min. Filtered the resultant solid to obtain crude compound. The crude compound was triturated with Acetonitrile (60 mL) to obtain Comp-56-OP (24 g, 52%) as an Off white solid.

### Step-e: 7-chloro-3-(4-(2,4-difluorophenoxy)piperidin-1-yl)pyrido[3,4-b]pyrazin-2-ol Hydrochloride (57-OP)

To the neat solid of comp-56-OP (24 g) added 4M HCl in Dioxane (480 mL, 20 Vol) at room temperature. The resulting suspension was stirred for 4-5h at 25-30° C. The progress of the reaction was monitored by TLC. After completion of the reaction, it was evaporated under vacuum to remove the solvent. The resulting solid taken into Diethyl ether (240 mL, 10 Vol) and stirred for 15-20 min at rt. The slurry was filtered to get compound-57-OP (18 g) as an off white solid. Which was dried under high vacuum for 10-15 min at room temperature to afford Comp-57-OP (HCl salt) (17 g, 84%) as an off white solid. The compound was used for next step.

### Step f: 2,7-dichloro-3-(4-(2,4-difluorophenoxy)piperidin-1-yl)pyrido[3,4-b]pyrazine (58-OP)

To a neat solid of Comp-57-OP (5.5 g, 14.03 mmol, 1 eq) was added SOCl₂ (27.5 mL, 5Vol) at room temperature. DMF (1 mL, 14.03 mmol, 1 eq) was added slowly to the reaction mixture. The heterogeneous reaction mixture was stirred at 80° C. for 16h. (During the course of the reaction clear solution was observed). The progress of the reaction was monitored by TLC. After completion of the reaction SOCl₂ was evaporated under vacuum to get crude as brown gum, which was directly loaded on to silica gel column (60-120) and eluted with 20-30% EtOAc in Hexane. The product fractions were concentrated under reduced pressure to afford compound 58-OP (3 g, 52%) as pale-yellow solid.

### Step-g: 7-chloro-3-(4-(2,4-difluorophenoxy) piperidin-1-yl)-2-(1-(fluoromethyl)-1H-pyrazol-4-yl)pyrido[3,4-b]pyrazine (62-OP)

In a 100 mL sealed tube at room temperature was added 1,4-Dioxane (40 mL) followed by Comp-58-OP (2 g, 4.86 mmol, 1 eq), Comp-D (1.09 g, 4.86 mmol, 1 eq) and K₂CO₃ (2.01 g, 14.58 mmol, 3 eq). The reaction mixture was degassed for 10-15 min with Nitrogen gas. After 15 minutes added Pd(dppfCl2). DCM (396 mg, 0.48 mmol, 0.1 eq) and further degassed for another 15 minutes. The reaction mixture was sealed and heated to 100-110° C. for 16h. the progress of the reaction was monitored by TLC. After completion of the reaction it was cooled to room temperature and diluted with EtOAc (50 mL), filtered through celite bed. The celite was washed with EtOAc (20 mL). Combined filtrates concentrated to get crude (3.5 g) as black gum, which was purified by RP-flash (C18, 40 g column) and 40-50% Acetonitrile in water as mobile phase. The product fractions were extracted with DCM (50 mL×2), the combined organic layer was dried over anhydrous Na₂SO₄, filtered, concentrated to afford Comp-62-OP (1.5 g, 65%) as brown solid.

### Step-h: 3-(4-(2,4-difluorophenoxy) piperidin-1-yl)-2-(1-(fluoromethyl)-1H-pyrazol-4-yl)-7-(2-methylprop-1-en-1-yl)pyrido[3,4-b]pyrazine (67-OP)

In a 100 mL sealed tube at room temperature was added 1,4-Dioxane/water (4:1) (20 mL) followed by Comp-62-OP (1.5 g, 3.15 mmol, 1 eq), Comp-F (1.09 g, 4.72 mmol, 1.5 eq) and K₂CO₃ (1.3 g, 9.45 mmol, 3 eq). The reaction mixture was degassed for 10-15 min with Nitrogen gas. After 15 minutes added Pd(dppfCl2). DCM (253 mg, 0.31 mmol, 0.1 eq) and further degassed for another 15 minutes. The reaction mixture was sealed and heated to 100-110° C. for 4 h. The progress of the reaction was monitored by TLC. After completion of the reaction, it was cooled to room temperature and diluted with EtOAc (50 mL), filtered through celite bed. The celite was washed with EtOAc (20 mL). Combined filtrates were concentrated to get crude (2.7 g) as black gum, which was purified by RP-flash (C18, 40 g column) and 40-50% Acetonitrile in water as mobile phase. The product fractions were extracted with DCM (50 mL×2), the combined organic layer was dried over anhydrous Na₂SO₄, filtered, concentrated to afford Comp-62-OP (0.8 g, 51%) as brown solid.

### Step-i: rel-(R)-1-(3-(4-(2,4-difluorophenoxy)piperidin-1-yl)-2-(1-(fluoromethyl)-1H-pyrazol-4-yl)pyrido[3,4-b]pyrazin-7-yl)-2-methylpropane-1,2-diol (Compound 593)

To the stirred solution of t-BuOH/Water (50 mL) at 0° C. was added 1-Admix (6.3 g, 8.08 mmol, 5 eq), followed by methane sulfonamide (153 mg, 1.61 mmol, 1 eq). The reaction mixture was stirred for 15 min then added slowly Comp-67-OP (0.8 g, 1.61 mmol, 1 eq, dissolved in 10 mL of t-BuOH). The reaction mixture was allowed to 25-30° C., stirred for 16 h. After 16h, LCMS showed 38% DP, 45% SM. (Note: Proceeded for work up and isolation without completion of the reaction). To the reaction mixture added water (50 mL) and extracted with DCM (100 mL×2). The combined organic layer was washed with sat. Na₂SO₃ solution (50 mL×2). (For each washing stirred for 30 min). Finally, the organic layer was dried over anhydrous Na₂SO₄, filtered and concentrated under reduced pressure to get crude (900 mg). The crude compound was purified by flash reverse phase column (40 g) using 70-80% Acetonitrile in water as mobile phase. Combined the product fractions were evaporated and re-dissolved in DCM (50 mL), dried over anhydrous Na₂SO₄, filtered and concentrated under reduced pressure to obtain Compound 153 (200 mg) as yellow solid, which showed LCMS: 96%, Chiral HPLC 96.5%+6.4% of another isomer. The obtained compound was further purified by Chiral PREP HPLC.

### Normal Phase Preparative (Chiral PREP) HPLC Conditions

| | |
|---|---|
| Column | Chiralpak AD-H (250*30) mm 5 micron |
| Buffer | 315 mg ammonium formate dissolved in 1000 ml of |
| preparation | Ethanol and sonicated the buffer for a minimum of 15 |
| | minutes |
| Mobile phase | Mixed 700 ml n-Hexane, 150 ml Buffer and 150 ml of |
| preparation | Isopropyl alcohol (70:15:15 v/v/v) |
| Elution mode | Isocratic |
| Flow Rate | 40 ml/min |
| Diluent | (1:1) v/v of Ethanol:Mobile phase |
| Run time | 25 min/injection |

Collected chiral prep fractions, evaporated under reduced pressure and dissolved in EtOAc (20 mL), washed with water (10 mL×2). Combined organic layers dried over anhydrous Na₂SO₄, filtered and concentrated to obtain compound as yellow gum. The compound was further subjected to lyophilization to afford Compound 593 (110 mg, 12.8%) as yellow solid.

Compound 593 (Peak-2): 1H NMR (400 MHz, DMSO-d6): δ9.06 (s, 1H), 9.00 (s, 1H), 8.46 (s, 1H), 7.87 (s, 1H), 7.37-7.26 (m, 2H), 7.06-6.99 (m, 1H), 6.36 (s, 1H), 6.23 (s, 1H), 5.49 (d, J=5.3 Hz, 1H), 4.66 (s, 1H), 4.58 (s, 1H), 4.56 (d, J=5.3 Hz, 2H), 3.54 (s, 2H), 3.18 (d, J=7.3 Hz, 2H), 2.09 (d, J=12.3 Hz, 2H), 1.97-1.86 (m, 2H), 1.07 (d, J=5.9 Hz, 6H). LCMS: m/z: 529.3 [M+H]; rt—3.44 min. Chiral HPLC: 99.97% (ee: 99.94%); rt—11.06 min. SOR: −18.28 (C=0.5%, MeOH)

### Example 60. Synthesis of 1-(2-(4-(2,4-difluorobenzylidene) piperidin-1-yl)-3-(1-methyl-1H-pyrazol-4-yl)-7,8-dihydropyrido[3,4-b]pyrazin-6(5H)-yl)ethan-1-one (Compound 542)

### Step a. tert-butyl 4-(2,4-difluorobenzylidene)piperidine-1-carboxylate (S91)

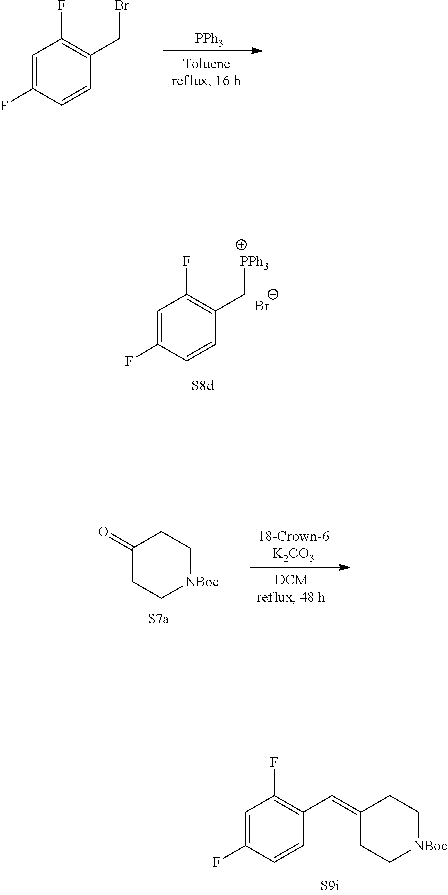

Phosphonium salt formation: In a 500 mL round-bottom flask, 2,4-difluorobenzyl bromide (17.8 g, 83.4 mmol) was added in one portion to a solution of thiphenylphosphine (22.1 g, 83.4 mmol) in anhydrous toluene (345 mL). The reaction mixture was heated to reflux and stirred for 16 hours. Afterwards, the mixture was cooled to room temperature, the precipitate formed was filtered, and dried on a high vacuum pump to provide compound S8d (36.7 g, 94%) as a white solid.

Wittig reaction: In a 1 L round-bottom flask, a mixture of S8d (32.7 g, 69.7 mmol), 1-Boc-4-Piperidone (S7a, 14.2 g, 69.7 mmol), potassium carbonate (12.5 g, 90.6 mmol), and 18-Crown-6 (3.76 g, 13.9 mmol) were dissolved in anhydrous DCM (500 mL). Then, the mixture was heated to reflux and stirred for 48 hours. Afterwards, the reaction mixture was cooled to room temperature and the solids were filtered. The filtrate was concentrated under reduced pressure and the obtained crude was purified by flash column chromatography (ethyl acetate in hexane, 0-10%, a gradient elution) to provide S9i (12.2 g, 57%) as a white solid.

### Step b. 3,7-dichloro-2-(4-(2,4-difluorobenzylidene)piperidin-1-yl)pyrido[3,4-b]pyrazine (S10f)

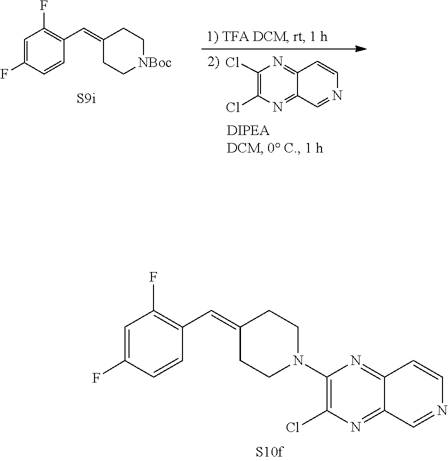 [SMILES: CC(C)(C)OC(=O)N1CCC(=Cc2ccc(F)cc2F)CC1 ; Clc1nc2ccncc2nc1Cl ; Fc1ccc(C=C2CCN(c3nc4ccncc4nc3Cl)CC2)c(F)c1]

Title compound was synthesized following one of the previous examples. S10f was obtained as yellow solid (128 mg, 76%).

### Step c. 2-(4-(2,4-difluorobenzylidene)piperidin-1-yl)-3-(1-methyl-1H-pyrazol-4-yl)pyrido[3,4-b]pyrazine (S11a)

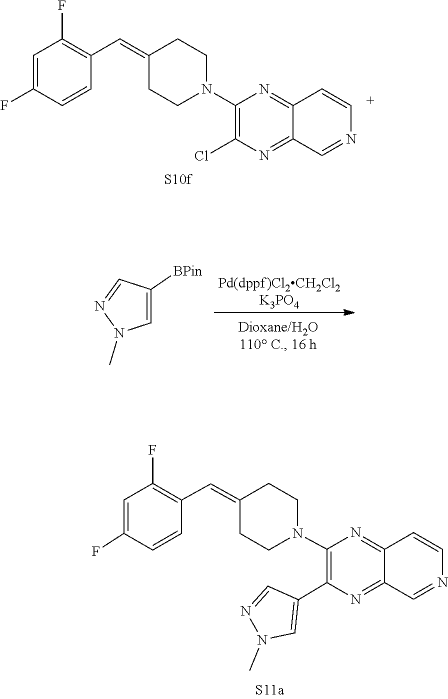

Step c procedure using Pd(dppf)Cl₂·CH₂Cl₂ as catalyst. S11a was obtained as orange solid (131 mg, 91%).

### Step d. 6-benzyl-2-(4-(2,4-difluorobenzylidene)piperidin-1-yl)-3-(1-methyl-1H-pyrazol-4-yl)-5,6,7,8-tetrahydropyrido[3,4-b]pyrazine (S3)

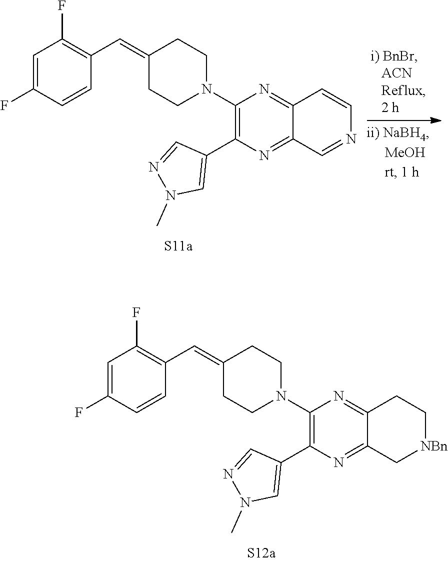 [SMILES: Cn1cc(-c2nc3cnccc3nc2N2CCC(=Cc3ccc(F)cc3F)CC2)cn1 ; Cn1cc(-c2nc3c(nc2N2CCC(=Cc4ccc(F)cc4F)CC2)CCN(Cc2ccccc2)C3)cn1]

Title compound was synthesized following one of the foregoing examples. Step b procedure. S12a was obtained as yellow oil (88.0 mg, 55%).

### Step d. 1-(2-(4-(2,4-difluorobenzylidene)piperidin-1-yl)-3-(1-methyl-1H-pyrazol-4-yl)-7,8-dihydropyrido[3,4-b]pyrazin-6(5H)-yl)ethan-1-one (Compound 542)

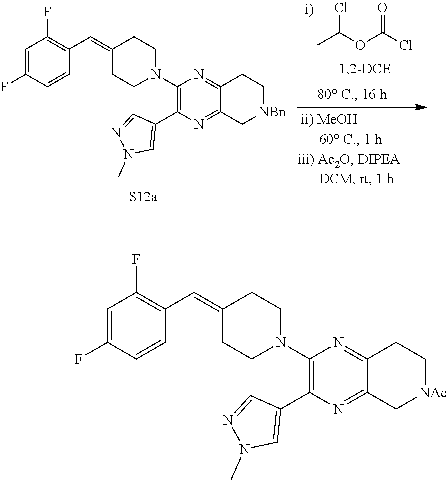

Title compound was synthesized following one of the foregoing examples. Step c procedure. Compound 542 was obtained as white-solid (16.3 mg, 85%).

### Example 61. Synthesis of 1-(2-{1-(2,4-difluorophenyl)-6-aza-6-spiro[2.5]octyl}-3-(1-methyl-4-pyrazolyl)-5,6,7,8-tetrahydro-1,4,6-triaza-6-naphthyl)-1-ethanone (Compound 660)

### Step b. tert-butyl 1,1-dichloro-2-(2,4-difluorophenyl)-6-azaspiro[2.5]octane-6-carboxylate (S9j)

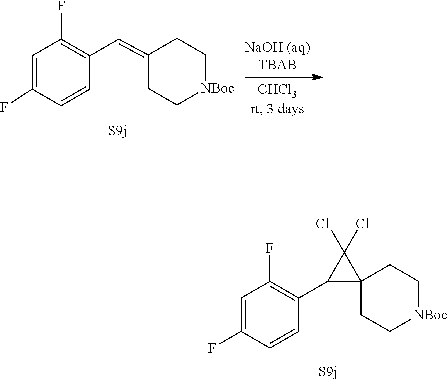 [SMILES: CC(C)(C)OC(=O)N1CCC(=Cc2ccc(F)cc2F)CC1 ; CC(C)(C)OC(=O)N1CCC2(CC1)C(c1ccc(F)cc1F)C2(Cl)Cl]

In a 100 mL round-bottom flask, compound S9i (2.00 g, 6.47 mmol) was dissolved in absolute CHCl₃ (32.0 mL), followed by the addition of tetrabutylammonium bromide (TBAB, 104 mg, 319 umol) and 43% aqueous solution of NaOH (23.6 ml). The reaction mixture was stirred vigorously at room temperature over 3 days. The reaction mixture was diluted with DCM and it was extracted with DCM (3×). The combined organic layers were washed with brine, dried over sodium sulfate and evaporated under reduced pressure. The obtained residue was purified by silica gel chromatography using Hexanes/Ethyl Acetate as eluents to obtain compound S9j (1.99 g, 79%) as a yellow oil.

### Step b. 3-chloro-2-(1-(2,4-difluorophenyl)-6-azaspiro[2.5]octan-6-yl)pyrido[3,4-b]pyrazine (S10g)

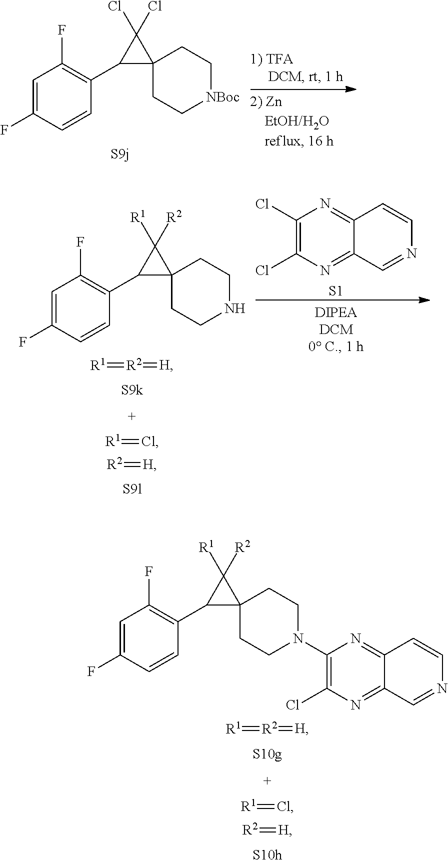

Step 1. Starting with compound S9j (1.99 g, 5.08 mmol), Boc-deprotection was performed following one of the foregoing examples. Step d procedure. No purification was done and the crude was directly used into the next step. Step 2. The oily crude was dissolved EtOH (16.3 mL), followed by the addition of water (2.00 mL) and Zinc powder (3.69 g, 56.4 mmol). Then, the reaction mixture was heated to reflux overnight. Afterwards, the zinc dust was filtered off through a short pad of Celite/sand and washed with additional Ethanol. The obtained clear solution was concentrated under reduced pressure and the obtained yellow-brown oil was directly used in the next step as a mixture of compounds S9k and S9l. Step 3. SNAr reaction was carried out using the previous crude and following one of the foregoing examples. Step d. compound S10g and S10h were obtained as an inseparable mixture (yellow oil, 728 mg).

### Step c. 2-(1-(2,4-difluorophenyl)-6-azaspiro[2.5]octan-6-yl)-3-(1-methyl-1H-pyrazol-4-yl)pyrido[3,4-b]pyrazine (S11b)

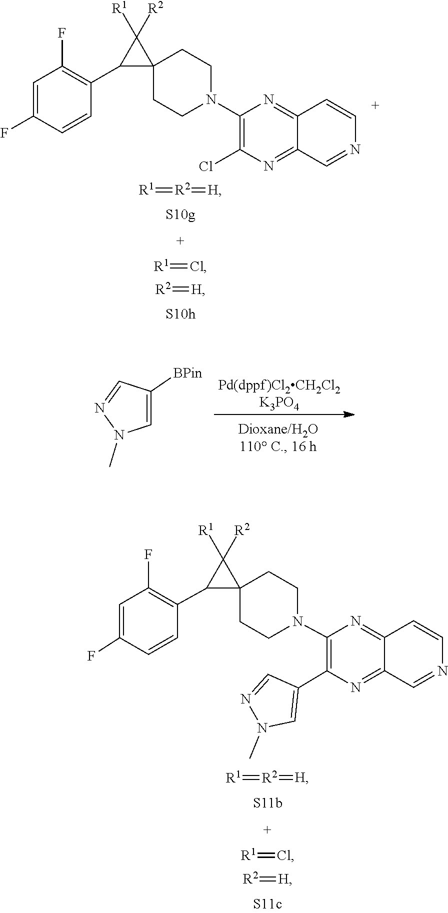

Title compound was synthesized following one of the foregoing examples. Step d procedure using Pd(dppf)Cl₂·CH₂Cl₂ as catalyst. S11a was obtained as a mixture with compound S11c (white solid, 447 mg).

### Step d. 1-(2-(1-(2,4-difluorophenyl)-6-azaspiro[2.5]octan-6-yl)-3-(1-methyl-1H-pyrazol-4-yl)-7,8-dihydropyrido[3,4-b]pyrazin-6(5H)-yl)ethan-1-one (Compound 660)

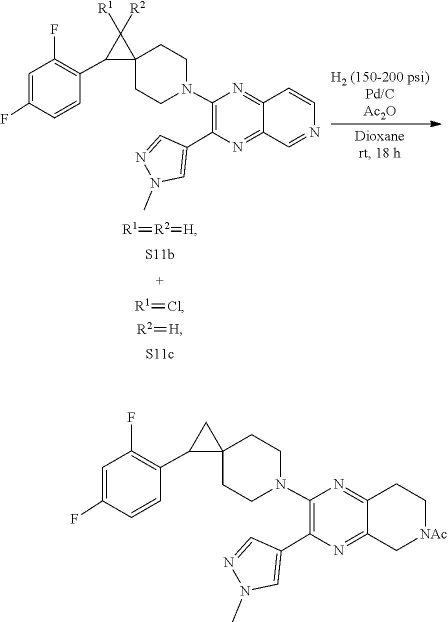

Using a high-pressure reactor equipped with a stir bar, the mixture of compounds S11b and S11c (447 mg) diluted in dioxane (12 mL) was transferred to the reaction container. Then, palladium over charcoal (10% wt, wet, 550 mg) was added followed by addition of acetic anhydride (1 mL). The reactor was sealed and placed under vacuum before backfilling with hydrogen and pressurizing to ˜150-200 psi. The reactor was set to stir at room temperature for 18 h (reaction completed as indicated by LCMS analysis). Afterwards, the reactor depressurised and opened, and the reaction mixture was filtered through Celite bed. The solvents were evaporated, and the obtained crude was directly purified by preparative HPLC using ammonium formate/ACN as eluents to obtain compound 660 (5 mg, 1%) as pale-yellow powder.

### Example 62. Synthesis of 1-(2-(4-((2,4-difluorophenyl)difluoromethyl)piperidin-1-yl)-3-(1-methyl-1H-pyrazol-4-yl)-7,8-dihydropyrido[3,4-b]pyrazin-6(5H)-yl)ethenone (Compound 698)

### Step a. (2,4-difluorophenyl)(pyridin-4-yl)methanone (S7d)

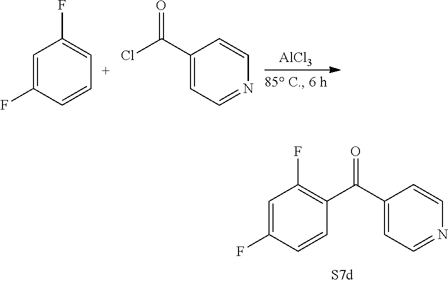

In a 100 mL round-bottom flask, to a mixture of 1,3-difluorobenzene (13.4 mL, 137 mmol) and isonicotinoyl chloride (5.00 g, 34.3 mmol), aluminium chloride (18.5 g, 137 mmol) was added portion-wise at room temperature. Then, the reaction mixture was stirred at 85° C. for 6 hours. Afterwards, the system was cooled to room temperature, and quenched with ice water. The aqueous suspension was extracted with ethyl acetate (×2). The aqueous layer was further treated with 40% NaOH (30 mL), and organics were extracted with ethyl acetate (2×100 mL). The combined organic layer was washed with brine, dried over sodium sulphate, and filtered. The filtrate was concentrated by rotary evaporation to afford S7d (5.20 g, 68%) as a yellow color solid.

### Step a. 4-((2,4-difluorophenyl)difluoromethyl)pyridine (S7e)

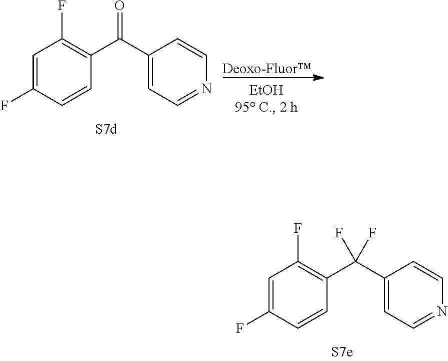

In a 20 mL microwave vial, compound S7d (500 mg, 2.28 mmol) was dissolved in ethanol (100 uL, 1.71 mmol), and bis(2-methoxyethyl)aminosulfur Trifluoride (Deoxo-Fluor™, 3.00 mL, 16.3 mmol) was added at room temperature. The vial was sealed, and the reaction mixture was stirred at 95° C. for 2 hours. Then, the mixture was cooled to room temperature and poured over ice. The aqueous suspension was extracted with dichloromethane (×2). The organic layer was washed with brine, dried over sodium sulphate, and filtered. The obtained residue was purified by flash column chromatography (ethyl acetate in hexane, 0-100%, gradient elution) to obtain S7e (231 mg, 41%) as yellow oil.

### Step a. 1-benzyl-4-((2,4-difluorophenyl)difluoromethyl)piperidine (S7f)

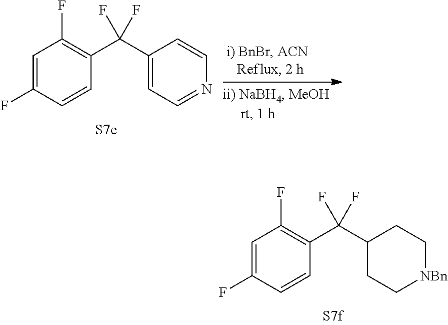 [SMILES: Fc1ccc(C(F)(F)c2ccncc2)c(F)c1 ; Fc1ccc(C(F)(F)C2CCN(Cc3ccccc3)CC2)c(F)c1]

Title compound was synthesized following one of the foregoing examples. Step b procedure. Compound S7f was obtained as yellow oil (2.13 g, 66%).

### Step a. 4-((2,4-difluorophenyl)difluoromethyl)piperidine (S9m)

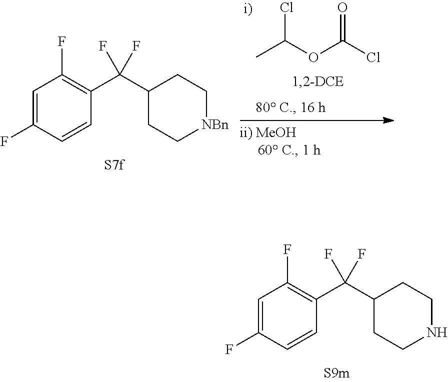

Title compound was synthesized following one of the foregoing examples. Step c procedure. Compound S7g was obtained as pale-yellow solid (500 mg, 68%).

### Step b. 3-chloro-2-(4-((2,4-difluorophenyl)difluoromethyl)piperidin-1-yl)pyrido[3,4-b]pyrazine (S10i)

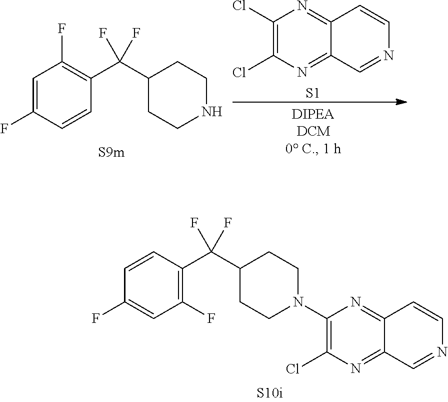

SNAr reaction was carried out following one of the foregoing examples. Step d conditions. Compound S10i was obtained and used as a crude in the next step.

### Step c. 2-(4-((2,4-difluorophenyl)difluoromethyl)piperidin-1-yl)-3-(1-methyl-1H-pyrazol-4-yl)pyrido[3,4-b]pyrazine (S11d)

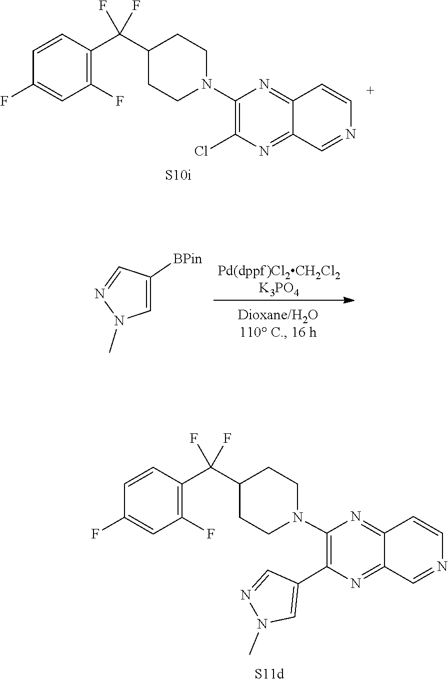

Title compound was synthesized following one of the foregoing examples. Step d procedure using Pd(dppf)Cl₂·CH₂Cl₂ as catalyst. Compound S11d was obtained as a yellow solid (570 mg, 62%).

### Step d. 6-benzyl-2-(4-((2,4-difluorophenyl)difluoromethyl)piperidin-1-yl)-3-(1-methyl-1H-pyrazol-4-yl)-5,6,7,8-tetrahydropyrido[3,4-b]pyrazine (S12b)

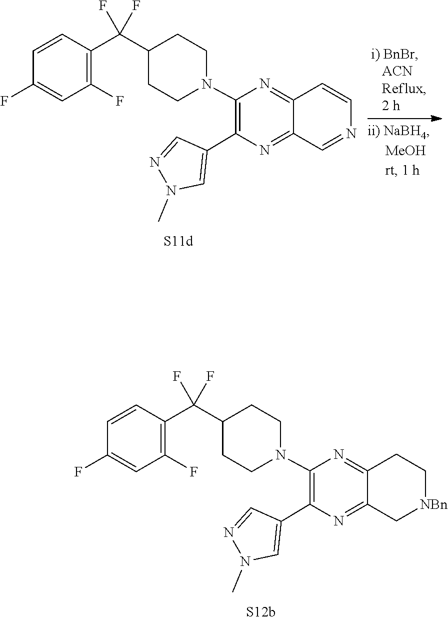 [SMILES: Cn1cc(-c2nc3cnccc3nc2N2CCC(C(F)(F)c3ccc(F)cc3F)CC2)cn1 ; Cn1cc(-c2nc3c(nc2N2CCC(C(F)(F)c4ccc(F)cc4F)CC2)CCN(Cc2ccccc2)C3)cn1]

Title compound was synthesized following one of the foregoing examples. Step b procedure. Compound S12b was obtained and used as a crude in the next step.

### Step d. 1-(2-(4-((2,4-difluorophenyl)difluoromethyl)piperidin-1-yl)-3-(1-methyl-1H-pyrazol-4-yl)-7,8-dihydropyrido[3,4-b]pyrazin-6(5H)-yl)ethan-1-one (Compound 698)

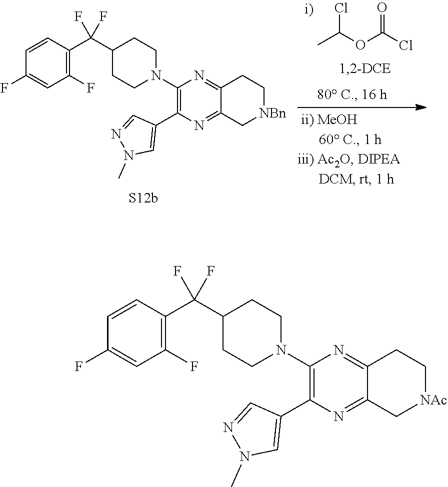

Title compound was synthesized following one of the foregoing examples. Step c procedure. Compound 698 was obtained as pale-yellow solid (26.7 mg, 15%).

### Example 63. Synthesis of 1-(2-(4-(2,4-difluorobenzyl)-4-hydroxypiperidin-1-yl)-3-(1-methyl-1H-pyrazol-4-yl)-7,8-dihydropyrido[3,4-b]pyrazin-6(5H)-yl)ethenone (Compound 699)

### Step a. tert-butyl 4-(2,4-difluorobenzyl)-4-hydroxypiperidine-1-carboxylate (S9n)

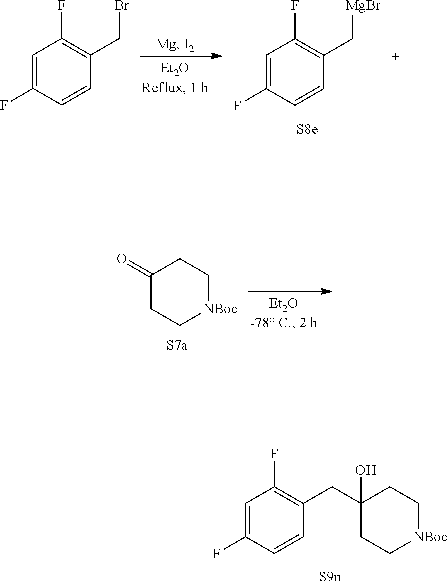

Step 1. In a flame-dried 100 mL 2-neck round-bottom flask under nitrogen atmosphere, a suspension of magnesium (2.40 g, 96.9 mmol), and iodine (260 mg, 1.02 mmol) in anhydrous ether (25.0 mL) was stirred at reflux for 15 minutes. After that, 2,4-difluorobenzyl bromide (5.08 mL, 39.2 mmol) was added dropwise over 15 minutes at reflux temperature while stirring. Then, the reaction mixture was stirred at reflux for 1 h. The resulting Grignard reagent (S8e) was used without any purification for the next step. Step 2. In a separate flame-dried 100 mL round-bottom flask under nitrogen atmosphere, a solution of 1-Boc-4-Piperidone (S7a, 7.97 g, 39.2 mmol) in anhydrous ether (200 mL) was cooled to −78° C. Then, the S8e ether solution from the previous step was transferred via syringe. The reaction mixture was let warm up to room temperature and stirred for 2 hours. Afterwards, the mixture was diluted in water (100 mL), and the aqueous layer was extracted with ethyl acetate (×2). The combined organic layer was washed with brine, dried over sodium sulphate, and filtered. The obtained residue was purified by flash chromatography using ethyl acetate/hexane as eluents to afford compound S9n (3.0 g, 23%) as a white solid.

### Step b. 1-(3-chloropyrido[3,4-b]pyrazin-2-yl)-4-(2,4-difluorobenzyl)piperidin-4-ol (S10j)

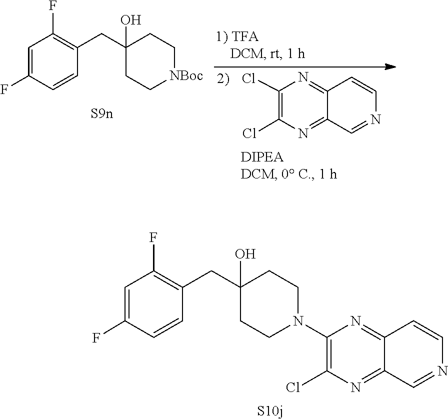 [SMILES: CC(C)(C)OC(=O)N1CCC(O)(Cc2ccc(F)cc2F)CC1 ; Clc1nc2ccncc2nc1Cl ; OC1(Cc2ccc(F)cc2F)CCN(c2nc3ccncc3nc2Cl)CC1]

Title compound was synthesized following one of the foregoing examples. Step d. Compound S10j was obtained as brown solid (580 mg, 96%).

### Step c. 4-(2,4-difluorobenzyl)-1-(3-(1-methyl-1H-pyrazol-4-yl)pyrido[3,4-b]pyrazin-2-yl)piperidin-4-ol (S11e)

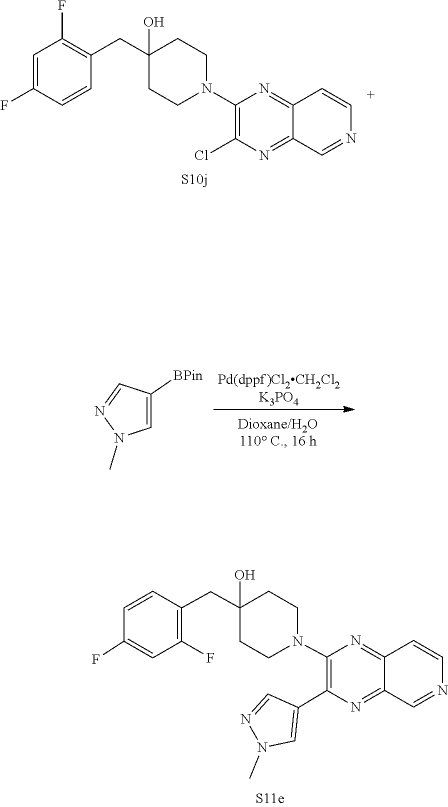

Title compound was synthesized following one of the foregoing examples. Step d procedure using Pd(dppf)Cl₂·CH₂Cl₂ as catalyst. Compound S11e was obtained as a yellow solid (578 mg, 74%).

### Step d. 1-(6-benzyl-3-(1-methyl-1H-pyrazol-4-yl)-5,6,7,8-tetrahydropyrido[3,4-b]pyrazin-2-yl)-4-(2,4-difluorobenzyl)piperidin-4-ol (S12c)

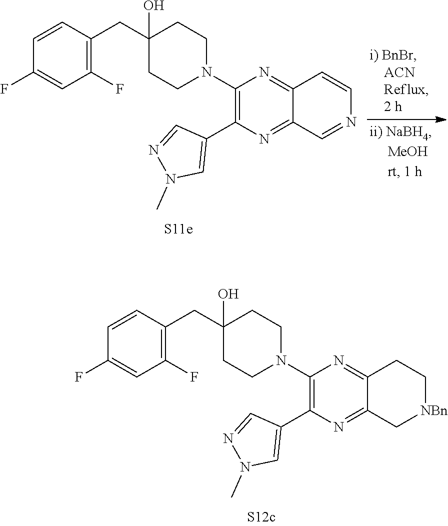 [SMILES: Cn1cc(-c2nc3cnccc3nc2N2CCC(O)(Cc3ccc(F)cc3F)CC2)cn1 ; Cn1cc(-c2nc3c(nc2N2CCC(O)(Cc4ccc(F)cc4F)CC2)CCN(Cc2ccccc2)C3)cn1]

Title compound was synthesized following one of the foregoing examples. Step b procedure. Compound S12c was obtained and used as a crude in the next step.

### Step d. 1-(2-(4-(2,4-difluorobenzyl)-4-hydroxypiperidin-1-yl)-3-(1-methyl-1H-pyrazol-4-yl)-7,8-dihydropyrido[3,4-b]pyrazin-6(5H)-yl)ethan-1-one (Compound 699)

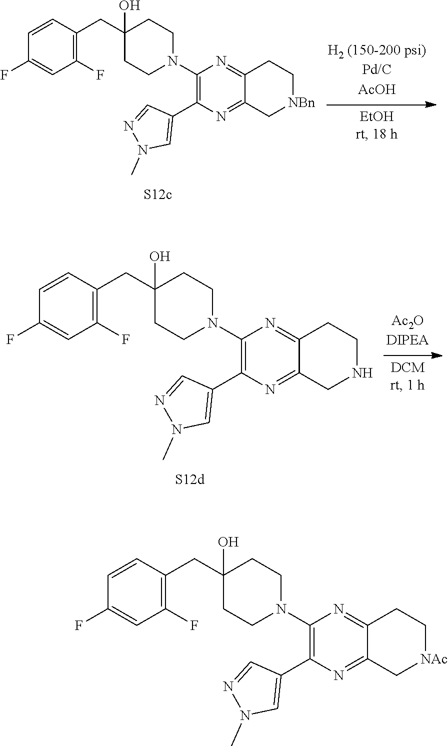

Compound S12d was synthesized following the hydrogenation procedure found in one of the foregoing examples. Step b. Afterwards, title compound was obtained following the acetylation procedure found in one of the foregoing examples. Step b. Compound 699 was obtained as white solid (89.0 mg, 34%).

### Example 64. Synthesis of rel-(R)-1-(3-(4-(2,4-difluorobenzylidene)piperidin-1-yl)-2-(1-(difluoromethyl)-1H-pyrazol-4-yl)pyrido[3,4-b]pyrazin-7-yl)-2-methylpropane-1,2-diol and rel-(S)-1-(3-(4-(2,4-difluorobenzylidene)piperidin-1-yl)-2-(1-(difluoromethyl)-1H-pyrazol-4-yl)pyrido[3,4-b]pyrazin-7-yl)-2-methylpropane-1,2-diol (Compound 767 and 768)

### Step a. 3,7-dichloropyrido[3,4-b]pyrazin-2-ol (S38a)

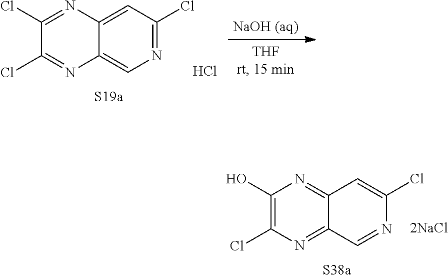 [SMILES: Clc1cc2nc(Cl)c(Cl)nc2cn1 ; Cl ; Oc1nc2cc(Cl)ncc2nc1Cl]

In a 500 mL round bottom flask, compound S19a (10.0 g, 36.9 mmol) was diluted in THF (148 mL). To the resulting suspension, 1 M aqueous solution of sodium hydroxide (73.8 mL, 73.8 mmol) was added. The system was stirred at room temperature for 15 min, where full conversion was observed by LCMS analysis. Afterwards, THF was evaporated in the rotovapor with no heat and the aqueous crude (pH verified to be 6-7) was then lyophilized overnight to obtain compound S38a (10.9 g, 89%) as a brown solid.

### Step b. 7-chloro-3-(4-(2,4-difluorobenzylidene)piperidin-1-yl)pyrido[3,4-b]pyrazin-2-ol (S39a)

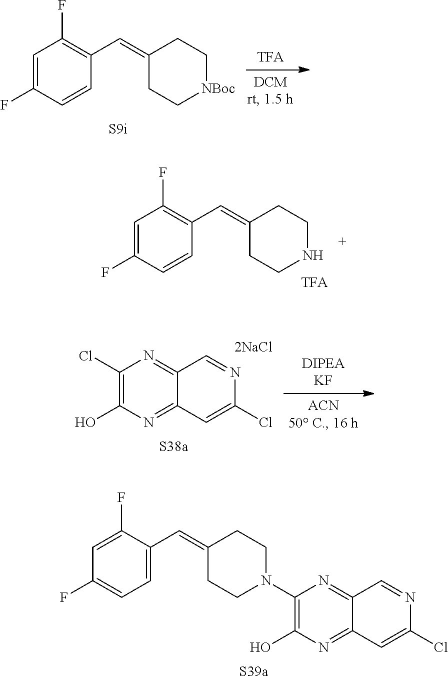 [SMILES: CC(C)(C)OC(=O)N1CCC(=Cc2ccc(F)cc2F)CC1 ; Fc1ccc(C=C2CCNCC2)c(F)c1 ; Oc1nc2cc(Cl)ncc2nc1Cl ; Oc1nc2cc(Cl)ncc2nc1N1CCC(=Cc2ccc(F)cc2F)CC1]

Step 1. Compound S9i (4.70 g, 15.2 mmol) was deprotected following one of the foregoing examples. Step c conditions. Step 2. The obtained crude (clear oil) was re-dissolved in ACN (78 mL) where compound S38a (4.70 g, 14.1 mmol) and potassium fluoride (892 mg, 15.2 mmol) were added. Then, N,N-diisopropylethylamine (13.4 mL, 76.0 mmol) was added dropwise and the system was stirred at 50° C. overnight. Afterwards, the solvent was evaporated to dryness using the rotovap. The obtained crude was directly purified by column chromatography over silica gel using DCM/MeOH as eluents, affording S39a (2.20 g, 37%) as brown solid.

### Step c. 2,7-dichloro-3-(4-(2,4-difluorobenzylidene)piperidin-1-yl)pyrido[3,4-b]pyrazine (S31c)

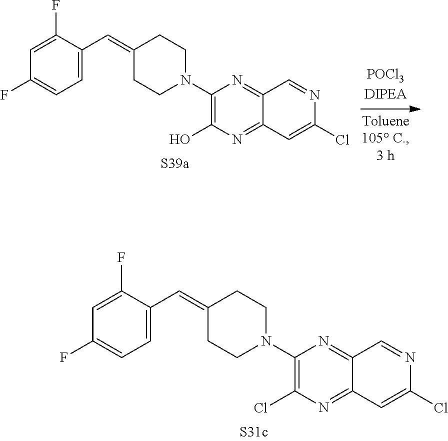 [SMILES: Oc1nc2cc(Cl)ncc2nc1N1CCC(=Cc2ccc(F)cc2F)CC1 ; Fc1ccc(C=C2CCN(c3nc4cnc(Cl)cc4nc3Cl)CC2)c(F)c1]

In a 25 mL round-bottom flask equipped with a stir bar, Compound S39a (600 mg, 1.54 mmol) was dissolved in anhydrous toluene (5.00 mL). The mixture was cooled to 0° C. and N,N-diisopropylethylamine (272 uL, 1.54 mmol) and phosphorus oxychloride (145 uL, 1.54 mmol) were added. Then, the system was heated 105° C. and stirred for 3 h. Afterwards, the reaction was cooled down to room temperature and the volatiles were evaporated under reduced pressure. The obtained residue was re-dissolved in anhydrous EtOAc (50 mL) and it was filtered. The obtained filtrate was evaporated off under reduced pressure to give S31c (732 mg, quant.) as dark brown solid.

### Step d. 7-chloro-3-(4-(2,4-difluorobenzylidene)piperidin-1-yl)-2-(1-(difluoromethyl)-1H-pyrazol-4-yl)pyrido[3,4-b]pyrazine (S32a)

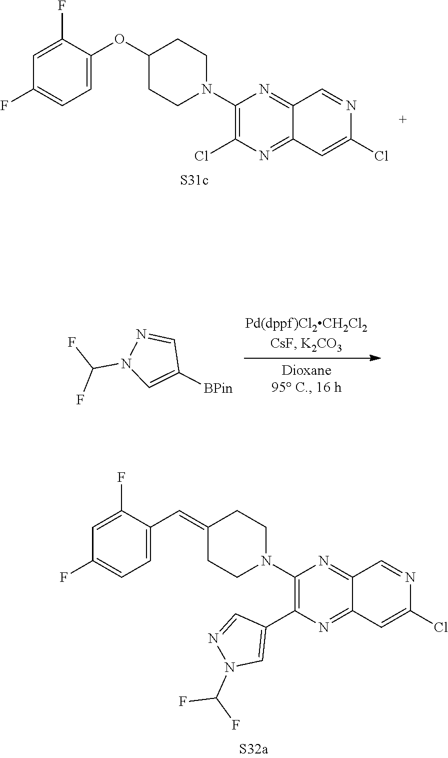

A microwave vial equipped with a stir bar was charged with S31c (400 mg, 0.982 mmol), Bis(diphenylphosphino)ferrocene]dichloropalladium(II), complex with dichloromethane (164 mg, 0.196 mmol), 1-(Difluoromethyl)-4-(4,4,5,5-tetramethyl-1,3,2-dioxaborolan-2-yl)-1H-pyrazole (272 mg, 1.08 mmol), cesium fluoride (151 mg, 982 umol) and potassium carbonate (416 mg, 2.95 mmol). Then, the solids mixture was diluted in anhydrous dioxane (3.9 mL) and the system was degassed using nitrogen gas bubbling (15 min) before sealing and heating at 95° C. overnight. Afterwards, the reaction mixture was filtrated through a pad of celite with EtOAc and DCM and concentrated. The obtained crude was purified by column chromatography over silica gel using DCM/MeOH as eluents, affording S32a (384 mg, 52%) as yellow fluffy solid.

### Step g. 3-(4-(2,4-difluorobenzylidene)piperidin-1-yl)-2-(1-(difluoromethyl)-1H-pyrazol-4-yl)-7-(2-methylprop-1-en-1-yl)pyrido[3,4-b]pyrazine (S35b)

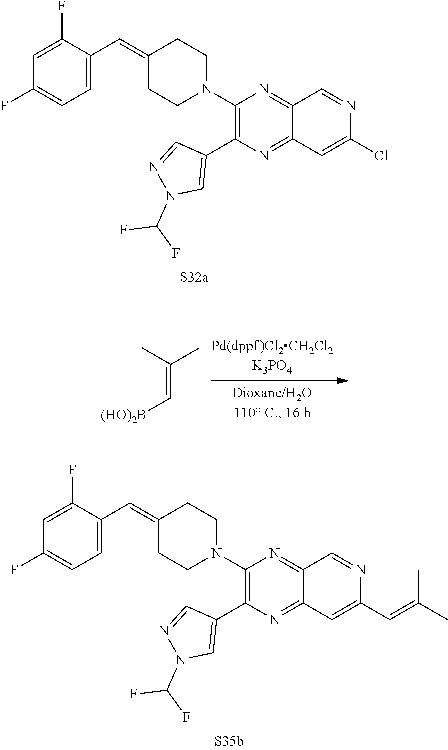

Title compound was synthesized following one of the foregoing examples. Step d procedure using Pd(dppf)Cl₂·CH₂Cl₂ as catalyst. S35a was obtained as yellow solid (117 mg, 29%).

### Step h. 3-(4-(2,4-difluorobenzylidene)piperidin-1-yl)-2-(1-(difluoromethyl)-1H-pyrazol-4-yl)-7-(3,3-dimethyloxiran-2-yl)pyrido[3,4-b]pyrazine (S35c)

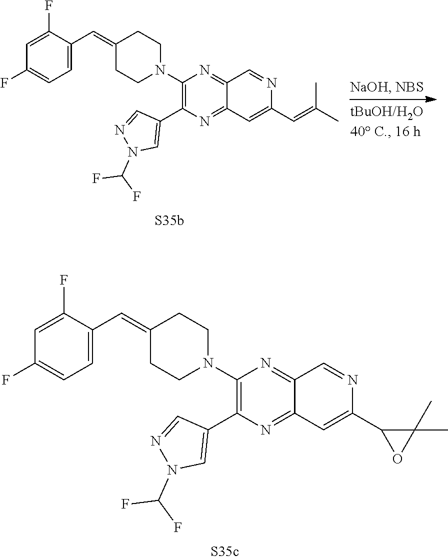 [SMILES: CC(C)=Cc1cc2nc(-c3cnn(C(F)F)c3)c(N3CCC(=Cc4ccc(F)cc4F)CC3)nc2cn1 ; CC1(C)OC1c1cc2nc(-c3cnn(C(F)F)c3)c(N3CCC(=Cc4ccc(F)cc4F)CC3)nc2cn1]

Title compound was synthesized following epoxidation procedure in one of the foregoing examples. Step e. Compound S35c was obtained as yellow powder (258 mg, 57%).

### Step h. (rac)-1-(3-(4-(2,4-difluorobenzylidene)piperidin-1-yl)-2-(1-(difluoromethyl)-1H-pyrazol-4-yl)pyrido[3,4-b]pyrazin-7-yl)-2-methylpropane-1,2-diol (S36a)

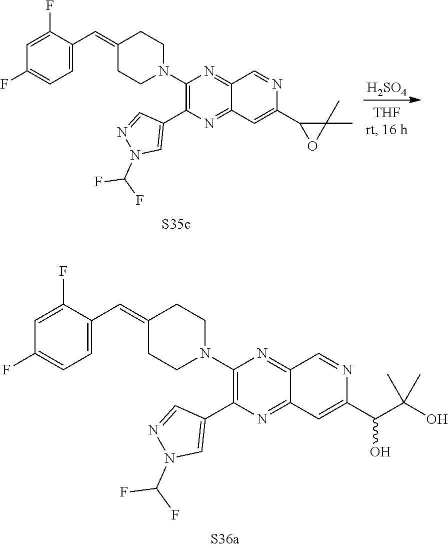 [SMILES: CC1(C)OC1c1cc2nc(-c3cnn(C(F)F)c3)c(N3CCC(=Cc4ccc(F)cc4F)CC3)nc2cn1 ; "H2SO4" ; CC(C)(O)C(O)c1cc2nc(-c3cnn(C(F)F)c3)c(N3CCC(=Cc4ccc(F)cc4F)CC3)nc2cn1]

Title compound was synthesized following epoxide opening procedure in one of the foregoing examples. Step e. Compound S36a was obtained as yellow powder (65.0 mg, 24%).

### Step h. rel-(R)-1-(3-(4-(2,4-difluorobenzylidene)piperidin-1-yl)-2-(1-(difluoromethyl)-1H-pyrazol-4-yl)pyrido[3,4-b]pyrazin-7-yl)-2-methylpropane-1,2-diol (Compound 766) and rel-(S)-1-(3-(4-(2,4-difluorobenzylidene)piperidin-1-yl)-2-(1-(difluoromethyl)-1H-pyrazol-4-yl)pyrido[3,4-b]pyrazin-7-yl)-2-methylpropane-1,2-diol (Compound 767)

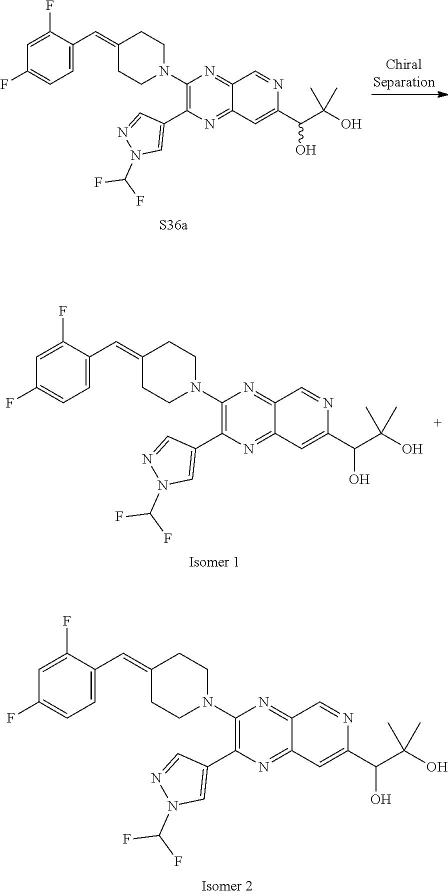 [SMILES: CC(C)(O)C(O)c1cc2nc(-c3cnn(C(F)F)c3)c(N3CCC(=Cc4ccc(F)cc4F)CC3)nc2cn1 ; CC(C)(O)C(O)c1cc2nc(-c3cnn(C(F)F)c3)c(N3CCC(=Cc4ccc(F)cc4F)CC3)nc2cn1 ; CC(C)(O)C(O)c1cc2nc(-c3cnn(C(F)F)c3)c(N3CCC(=Cc4ccc(F)cc4F)CC3)nc2cn1]

Compound S36a (51.0 mg, 0.100 mmol) was submitted for chromatographic chiral separation using a Waters SFC Prep 150 system under the conditions described below:

| | |
|---|---|
| Column | Daicel |
| Manufacturer | |
| Column | ChiralPak IG, LC Column 250 × 10 mm, Particle size 5 |
| Description | μm, Pore size 1000 Å |
| Stationary | Amylose tris(3-chloro-5-methylphenylcarbamate) |
| phase | |
| Mode | Isocratic |
| Mobile phase | 60% MeOH + 0.1% NH₄OH |
| | 40% supercritical CO₂ |
| Flow rate | 10 mL/min |
| Backpressure | 150 bar |
| Column | 40° C. |
| Temperature | |
| Run time (min) | 20 |
| Retention | Isomer 1 - compound 766 = 11.06 min |
| time (min) | Isomer 2 - compound 767 = 15.25 min |

The obtained fractions were evaporated to obtain compounds compound 766—Isomer 1 (14.7 mg, 29%, 99.3% ee) and compound 767—Isomer 2 (14.3 mg, 28%, 99.0% ee) as yellow powders.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

What is claimed is:

1. A compound of Formula (I), or a pharmaceutically acceptable salt thereof: 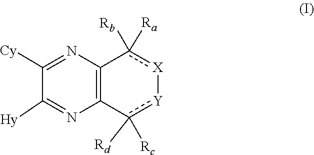
wherein
Rₐ, R_{b}, R_{c}, and R_{d} are each independently absent, H, alkyl, alkenyl, halogen, —CN, —CH₂NRₑR_{f}, —NRₑR_{f}, —NRₑC(═O)R_{f}, —NRₑC(═O)NR_{f}R_{g}, —NRₑC(═O)OR_{f}, —NRₑSO₂R_{f}, —OC(═O)NRₑR_{f}, —ORₑ, —SRₑ, —SORₑ, —SO₂Rₑ, —SO₂ORₑ, —SO₂NRₑR_{f}, —C(═O)Rₑ, —C(═O)ORₑ, —C(═O)C(CH₃)₂OH, —C(═O)NRₑR_{f}, spirocyclyl, spiroheterocyclyl, carbocyclyl, or heterocyclyl; Rₐ and R_{b} together with the atoms to which they are attached form ═NSO₂Rₑ, ═O, carbocyclyl, or heterocyclyl; R_{c} and R_{d} together with the atoms to which they are attached form ═NSO₂Rₑ, ═O, carbocyclyl, or heterocyclyl; or Rₐ and R_{c} together with the atoms to which they are attached form a carbocyclyl or heterocyclyl; Rₐ and Y together with the atoms to which they are attached form a carbocyclyl or heterocyclyl; R_{c} and X together with the atoms to which they are attached form a carbocyclyl or heterocyclyl;
each of X and Y is independently O, N, NRₑ, N(C(Rₑ)₂C(═O)NHR_{f}), N(C(═O)Rₑ), N(C(═O)ORₑ), NC(═O)C(Rₑ)₃, NC(═O)C(Rₑ)₂(Rₑ)₃, S, S(═O), S(═O)₂, S(═O)(═NRₑ), S(═NRₑ)₂, C(CN), C(CN)Rₑ, C(C(═O)Rₕ), C(Rₕ), NS(O)₂NRₑRₕ, NS(O)NRₑRₕ, C(═NSO₂Rₑ), or C(Rₕ)₂; provided that X and Y cannot both be selected from C(CN), C(CN)Rₑ, C(C(═O)Rₑ) and C(Rₕ);
each Rₑ, R_{f}, and R_{g} is independently H, halogen, —OH, —CN, —OCHF₂, —C(CH₃)₂OH, -haloalkyl, —CH₂OH, CH(CH₃)CN, —C((CH₃)₂CN), C(═O)CH₃, —NH₂, NH(alkyl), —CH₂CH₂OCH₃, —CH₂CH(OH)CH₂OH, alkoxy, alkyl, carbocyclyl, or heterocyclyl,
each Rₕ is independently H, halogen, OH, —CN, alkyl, —C(Rₑ)₃, —C(Rₑ)₂C(Rₑ)₃, —CH₂C(Rₑ)₂C(Rₑ)₃, C(═O)C(Rₑ)₃, —OCH₂C(Rₑ)₃, —NHCH₂C(Rₑ)₃, —N(alkyl)CH₂C(Rₑ)₃, —O-heterocyclyl, alkoxy, haloalkoxy, —NRₑR_{f}, —NRₑC(═O)R_{f}, —NRₑC(═O)NR_{f}R_{g}, —CH₂NRₑC(═O)NR_{f}R_{g}, —CH₂C(═O)NRₑR_{f}, —NRₑC(═O)OR_{f}, —NRₑSO₂R_{f}, —OC(═O)NRₑR_{f}, —ORₑ, —SRₑ, —SORₑ, —SO₂Rₑ, —SO₂ORₑ, —SO₂NRₑR_{f}, —C(═O)Rₑ, —C(═O)ORₑ, —C(═O)NRₑR_{f}, carbocyclyl, aryl or heterocyclyl; or two Rₕ attached to the same carbon atom together with the atoms to which they are attached form ═NSO₂Rₑ, ═O, carbocyclyl, heteroaryl or heterocyclyl;
Hy is a carbon-linked 3-, 4-, 5- or 6-membered carbocyclyl; a carbon-linked 3-10-membered monocyclic heterocyclyl; or a carbon-linked 3-10-membered bicyclic heterocyclyl; and
Cy is -A-L-B or -A=L-B;
wherein
A is a nitrogen-containing heterocyclylene linked by a nitrogen atom to 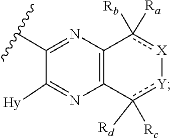
B is a 5- to 7-membered heterocyclyl or a 5- to 7-membered carbocyclyl; and
L is absent, C(Rₕ), C(Rₕ)₂, OC(Rₕ)₂, C(═O), C(═NSO₂Rₑ), C(═O)N(Rₑ), NRₑ, O, S, N, S(═O), S(═O)₂, S(═O)(═NRₑ), or S(═NRₑ)₂, wherein when L is absent or NRₑ, B is aryl or heteroaryl.

2. The compound of claim 1, having Formula (Ib) 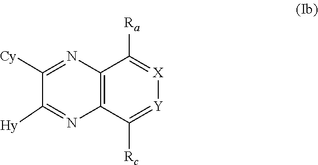
wherein
Rₐ and R_{c} are each independently H, alkyl, alkenyl, halogen, —CN, —CH₂NRₑR_{f}, —NRₑR_{f}, —NRₑC(═O)R_{f}, —NRₑC(═O)NR_{f}R_{g}, —NRₑC(═O)OR_{f}, —NRₑS(═O)₂R_{f}, —OC(═O)NRₑR_{f}, —ORₑ, —SRₑ, —S(═O)Rₑ, —S(═O)₂Rₑ, —S(═O)₂ORₑ, —S(═O)₂NRₑR_{f}, —C(═O)Rₑ, —C(═O)ORₑ, —C(═O)C(CH₃)₂OH, —C(═O)NRₑR_{f}, spirocyclyl, spiroheterocyclyl, carbocyclyl, or heterocyclyl;
each of X and Y is independently N, C(CN), C(C(═O)Rₑ), or C(Rₕ);
each Rₑ, R_{f}, and R_{g} is independently H, halogen, —OH, —CN, —OCHF₂, —C(CH₃)₂OH, -haloalkyl, —CH₂OH, CH(CH₃)CN, —C((CH₃)₂CN), C(═O)CH₃, —NH₂, NH(alkyl), —CH₂CH₂OCH₃, —CH₂CH(OH)CH₂OH, alkoxy, alkyl, carbocyclyl, or heterocyclyl,
each Rₕ is independently H, halogen, OH, —CN, alkyl, —C(Rₑ)₃, —C(Rₑ)₂C(Rₑ)₃, —CH₂C(Rₑ)₂C(Rₑ)₃, C(═O)C(Rₑ)₃, —OCH₂C(Rₑ)₃, —NHCH₂C(Rₑ)₃, —N(alkyl)CH₂C(Rₑ)₃, —O-heterocyclyl, alkoxy, haloalkoxy, —NRₑR_{f}, —NRₑC(═O)R_{f}, —NRₑC(═O)NR_{f}R_{g}, —CH₂NRₑC(═O)NR_{f}R_{g}, —CH₂C(═O)NRₑR_{f}, —NRₑC(═O)OR_{f}, —NRₑSO₂R_{f}, —OC(═O)NRₑR_{f}, —ORₑ, —SRₑ, —SORₑ, —SO₂Rₑ, —SO₂ORₑ, —SO₂NRₑR_{f}, —C(═O)Rₑ, —C(═O)ORₑ, —C(═O)NRₑR_{f}, carbocyclyl, aryl or heterocyclyl; or two Rₕ attached to the same carbon atom together with the atoms to which they are attached form ═NSO₂Rₑ, ═O, carbocyclyl, heteroaryl or heterocyclyl;
Hy is a carbon-linked 3-, 4-, 5- or 6-membered carbocyclyl; a carbon-linked 3-10-membered monocyclic heterocyclyl; or a carbon-linked 3-10-membered bicyclic heterocyclyl; and
Cy is -A-L-B or -A=L-B;
wherein
A is a nitrogen-containing heterocyclylene linked by a nitrogen atom to 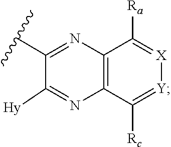
and
B is a 5- to 7-membered heterocyclyl or a 5- to 7-membered carbocyclyl; and
L is absent, C(Rₕ), C(Rₕ)₂, OC(Rₕ)₂, C(═O), C(═O)N(Rₑ), C(═NSO₂Rₑ), NRₑ, O, S, N, S(═O), S(═O)₂, S(═O)(═NRₑ), or S(═NRₑ)₂, wherein when L is absent or NRₑ, B is aryl or heteroaryl.

3. The compound of any one of claims 1-2, wherein Rₐ and R_{c} are H.

4. The compound of claim 1, wherein Rₐ, R_{b}, R_{c}, and R_{d} are H.

5. The compound of any one of claims 1-4, wherein one of X and Y is C(Rₕ), and the other one of X and Y is N.

6. The compound of any one of claims 1-5, wherein X is C(Rₕ), and Y is N.

7. The compound of any one of claims 1-5, wherein Y is C(Rₕ), and X is N.

8. The compound of any one of claims 1-7, wherein Rₕ is H, alkyl, heterocyclyl, —C(Rₑ)₃, —C(Rₑ)₂C(Rₑ)₃, —CH₂C(Rₑ)₂C(Rₑ)₃, —OCH₂C(Rₑ)₃, —NHCH₂C(Rₑ)₃, —N(alkyl)CH₂C(Rₑ)₃ or C(═O)C(Rₑ)₃.

9. The compound of any one of claims 1-7, wherein Rₕ is —C(Rₑ)₃, —C(Rₑ)₂C(Rₑ)₃, —CH₂C(Rₑ)₂C(Rₑ)₃, —OCH₂C(Rₑ)₃ or C(═O)C(Rₑ)₃.

10. The compound of any one of claims 1-7, wherein Rₕ is

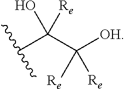

11. The compound of any one of claims 1-7, wherein Rₕ is

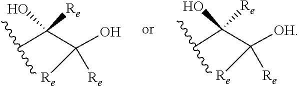

12. The compound of any one of any one of claims 8-11, wherein Rₑ is H, alkyl, OH, alkoxy, halogen, CN, or cycloalkyl.

13. The compound of any one of claims 1-7 and 12, wherein Rₕ is

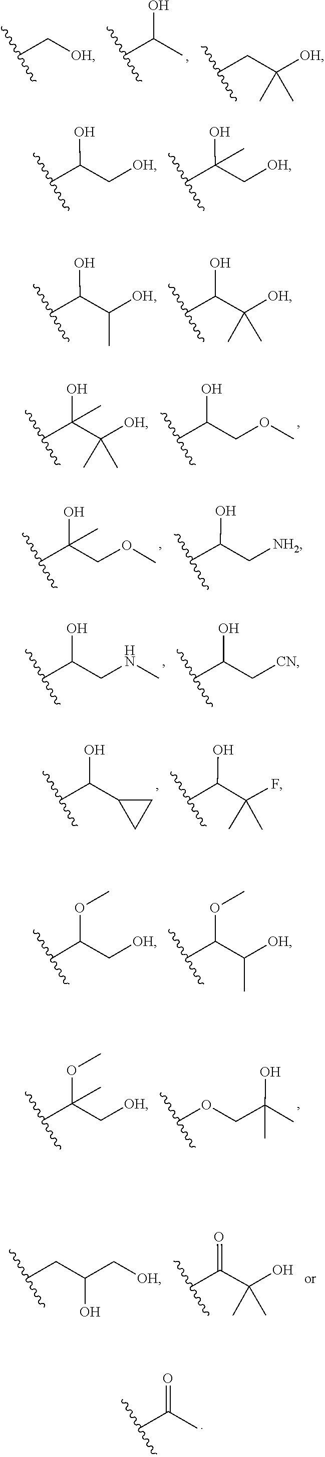

14. The compound of any one of claims 1-7 and 12, wherein Rₕ is

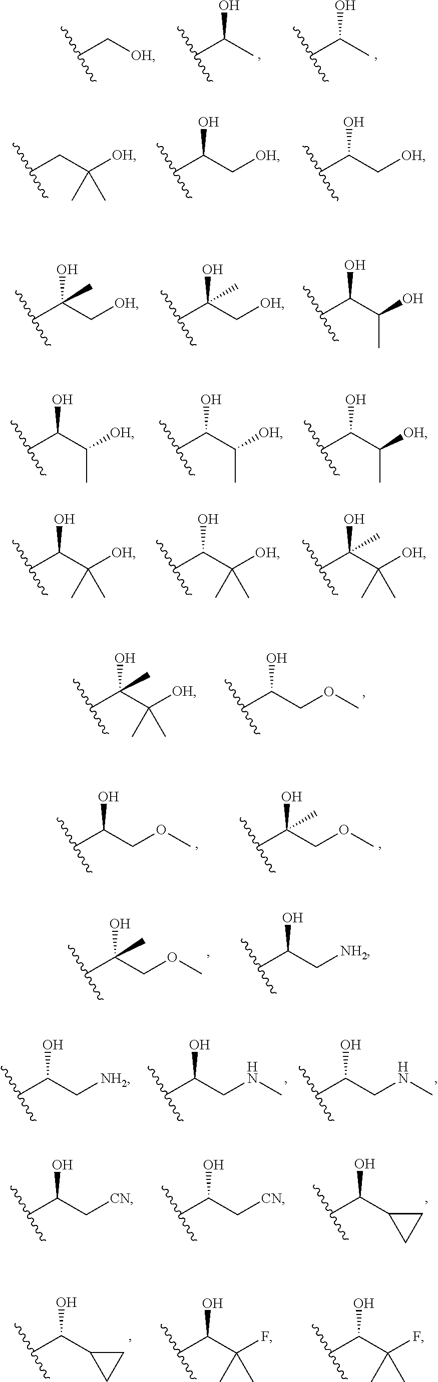

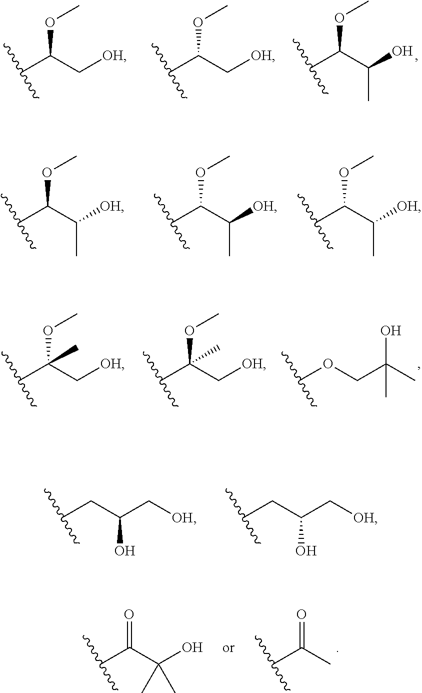

15. The compound of any one of claims 1-7 and 12, wherein Rₕ is

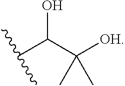 [SMILES: *C(O)C(C)(C)O]

16. The compound of any one of claims 1-7 and 12, wherein Rₕ is

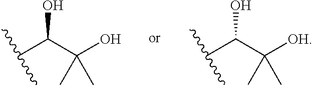

17. The compound of any one of claims 1-7 and 12, wherein one of X and Y is N, and the other one of X and Y is C(Rₕ), and Rₕ is —C(Rₑ)₃, —C(Rₑ)₂C(Rₑ)₃, —CH₂C(Rₑ)₂C(Rₑ)₃, —OCH₂C(Rₑ)₃ or C(═O)C(Rₑ)₃.

18. The compound of any one of claims 1-7 and 12, wherein one of X and Y is N, and the other one of X and Y is C(Rₕ), and Rₕ is

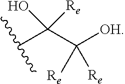

19. The compound of any one of claims 1-7 and 12, wherein one of X and Y is N, and the other one of X and Y is C(Rₕ), and Rₕ is

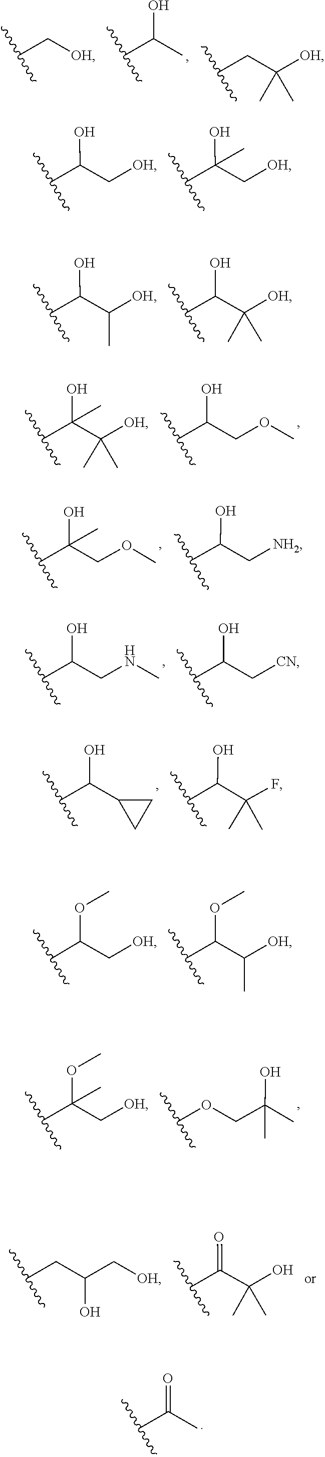

20. The compound of any one of claims 1-7 and 12, wherein one of X and Y is N, and the other one of X and Y is C(Rₕ), and Rₕ is

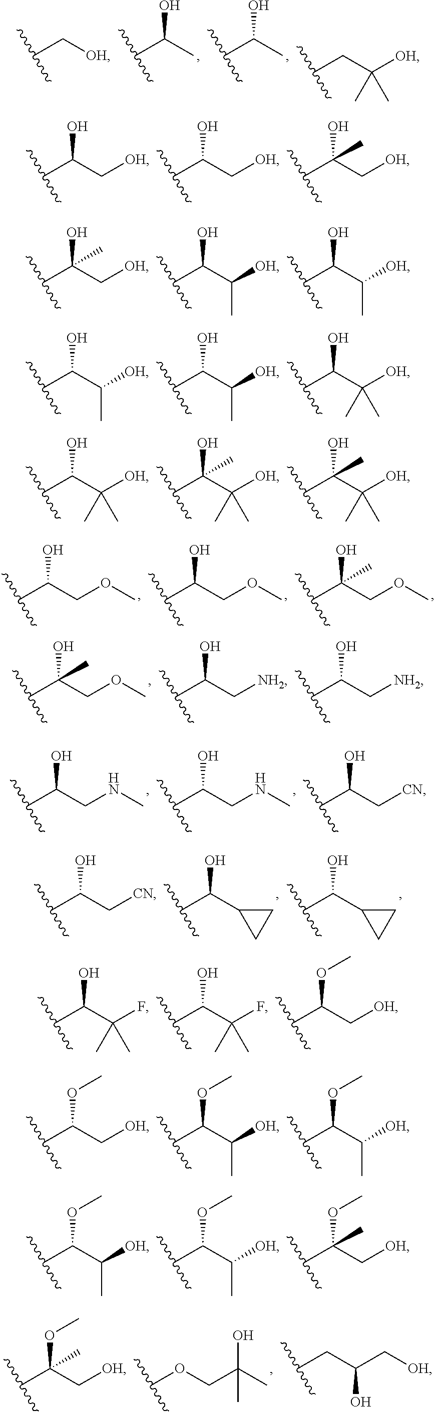

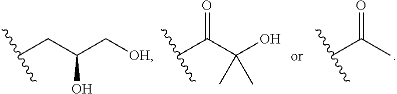

21. The compound of any one of claims 1-7 and 12, wherein one of X and Y is N, and the other of X and Y is C(Rₕ) and Rₕ is

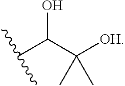 [SMILES: *C(O)C(C)(C)O]

22. The compound of any one of claims 1-7 and 12, wherein one of X and Y is N, and the other of X and Y is C(Rₕ) and Rₕ is

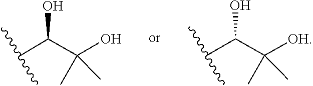

23. The compound of claim 1, wherein one of X and Y is N(C(═O)CH₃), and the other one of X and Y is CH₂.

24. The compound of claim any one of claims 1-23, wherein Hy is 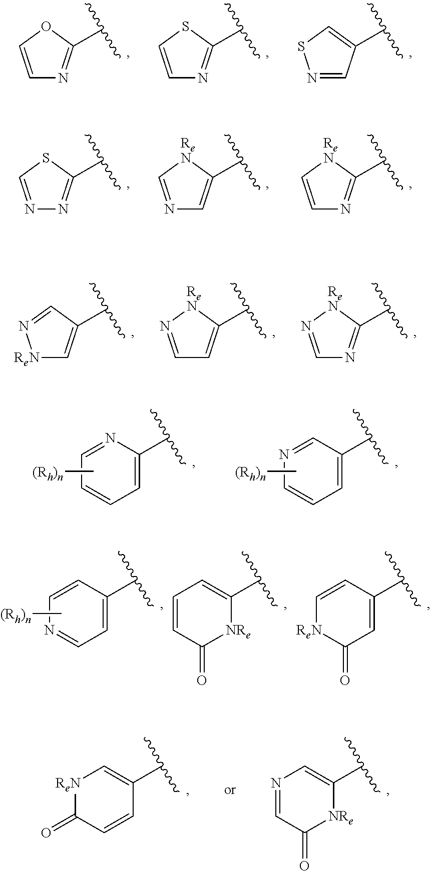
wherein
Rₕ is halogen, OH, alkyl, NH₂, NH(C₁₋₆ alkyl), CN, —CH₂OCH₃, —OCH₃, —OCH₂CH₃, —OC(CH₃)₃, OCHF₂, OCF₃, —C(═O)CH₃, —CH₂C(═O)NHCH₃, —C(═O)OC(CH₃)₃, O-heterocycle, C₃₋₆ cycloalkyl, CH₂CH₂CN, CH₂CN, C(CH₃)₂CN, CH(CH₃)CN, CH₂CF₃, —CH₂OCH₃, CH₂CH₂OCH₃, CH₂CH₂OH, CH(OH)CHF₂, aryl, heterocyclyl,
n is 0, 1, 2, or 3; and
Rₑ is H, OCH₃, CH₃, CH₂CH₃, CH₂F, CHF₂, C₃₋₆ cycloalkyl, —C(═O)CH₃ or heterocyclic.

25. The compound of any one of claims 1-24, wherein Hy is

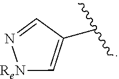

26. The compound of any one of claims 24-25, wherein Rₑ is CH₂F, CHF₂, CH₃, CH₂CH₃, or cyclopropyl.

27. The compound of any one of claims 1-26, wherein Cy is -A-L-B.

28. The compound of any one of claim 1-27, wherein Cy is 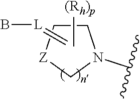
wherein p=0, 1, 2, 3 or 4; n′=0, 1 or 2; Z is C(Rₕ), C(Rₕ)₂, C, N, N(Rₑ), O, S, S(═O) or S(═O)₂, Rₕ is H, halogen, alkyl, ═O, alkoxy or CH₂CH₂OCH₃, and R is H or alkyl.

29. The compound of any one of claims 1-28, wherein -A-L-B is 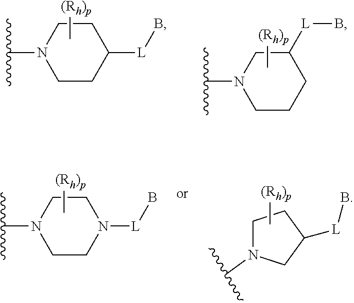
wherein p is 0, 1, 2, 3, or 4, and Rₕ is H, halogen, alkyl, alkoxy or CH₂CH₂OCH₃, or two Rₕ attached to the same carbon together with the atoms to which they are attached form ═O.

30. The compound of any one of claims 1-29, wherein -A-L-B is

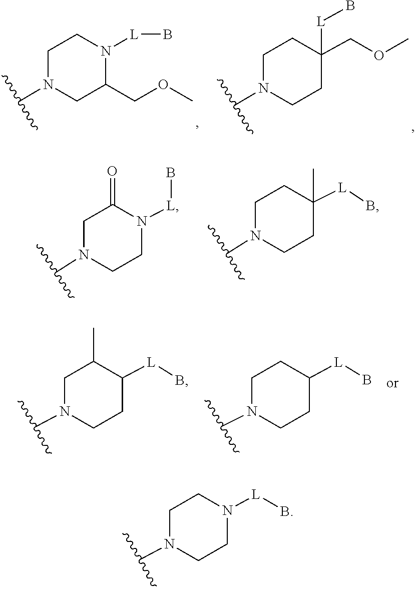

31. The compound of any one of claims 1-30, wherein -A-L-B is

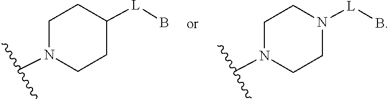

32. The compound of any one of claims 1-31, wherein -A-L-B is

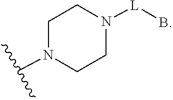

33. The compound of any one of claims 1-32, wherein L is absent, O, C(Rₕ), C(Rₕ)₂, —OC(Rₕ)₂—, C(═O), C(═O)N(Rₑ), NRₑ or N, Rₕ is H, halogen, alkyl, haloalkyl, OH or CH₂OH, and Rₑ is H or alkyl.

34. The compound of any one of claims 1 to 32, wherein L is 0, CH₂, OCH₂, C(═O), NH, NCH₃, or N.

35. The compound of any one of claims 1-32, wherein L is O or CH₂.

36. The compound of any one of claims 1-32, wherein L is CH₂.

37. The compound of any one of claims 1-36, wherein B is 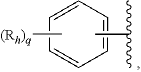
wherein q is 0, 1, 2, 3, 4, or 5, and Rₕ is halogen, alkyl, alkoxy, CN or S(═O)₂alkyl.

38. The compound of any one of claims 1-36, wherein B is

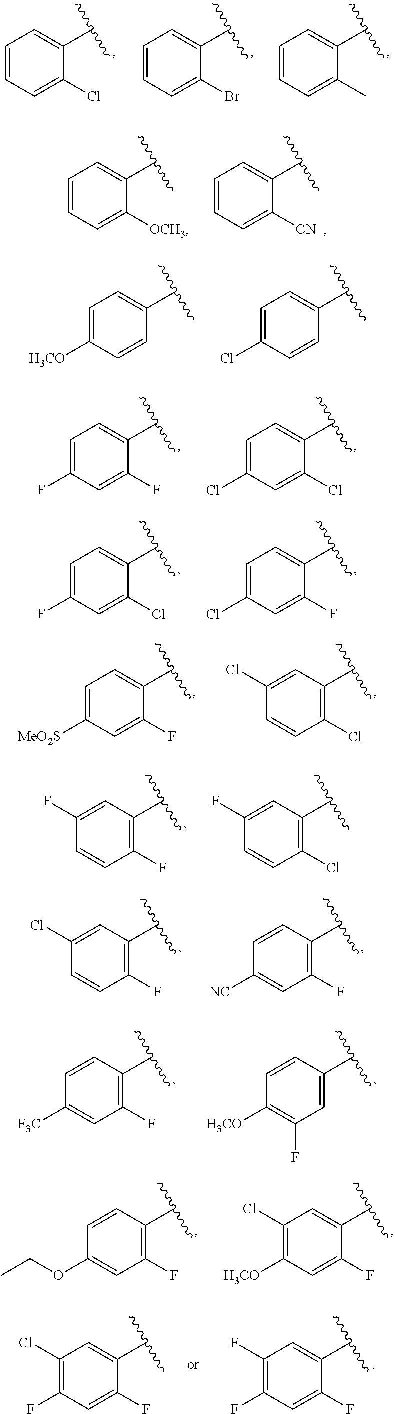

39. The compound of any one of claims 1-36, wherein B is

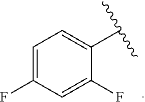 [SMILES: *c1ccc(F)cc1F]

40. The compound of any one of claims 1-26 wherein Cy is 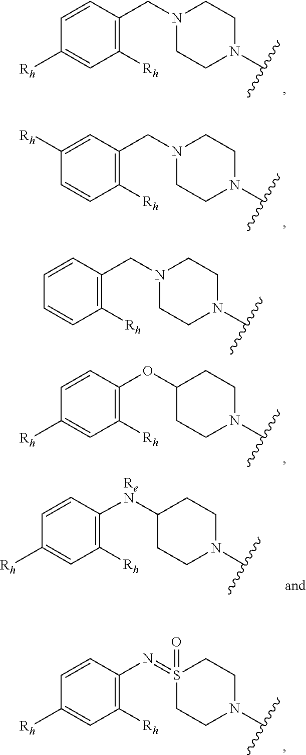
wherein Rₕ is halogen, alkoxy, CN, alkyl, haloalkyl S(═O)₂alkyl, or CH₂CH₂OCH₃.

41. The compound of any one of claims 1-26 or 40, wherein Cy is

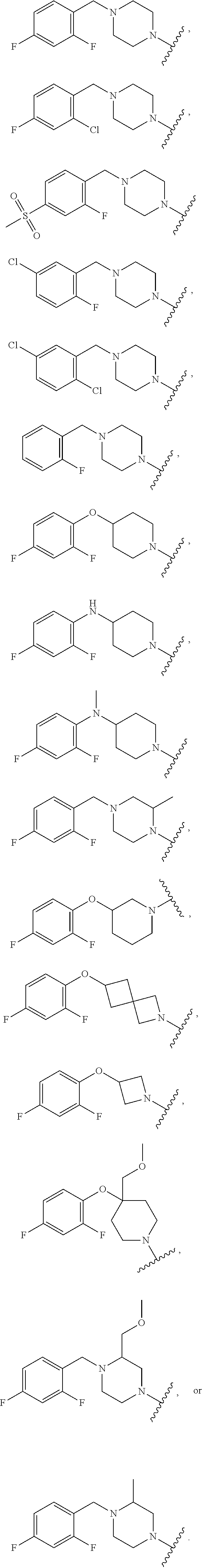

42. The compound of any one of claims 1-26 or 40-41, wherein Cy is

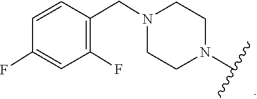 [SMILES: *N1CCN(Cc2ccc(F)cc2F)CC1]

43. A compound selected from

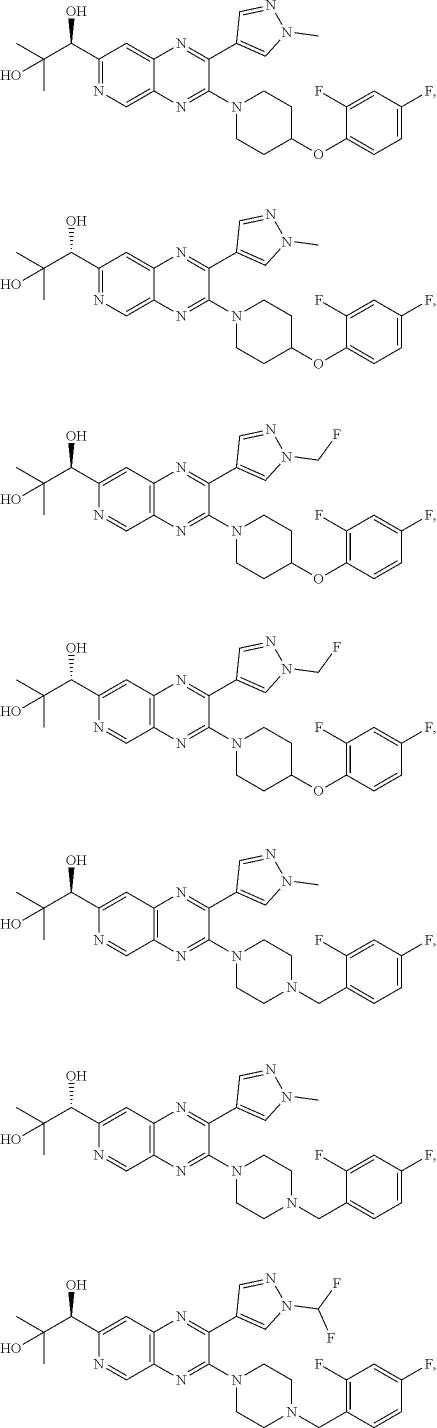

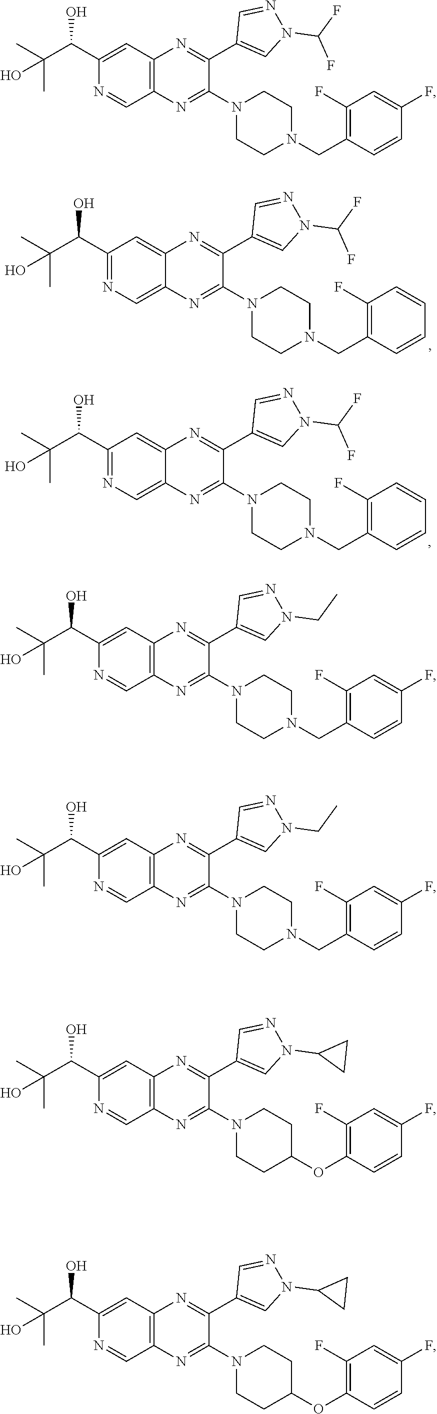

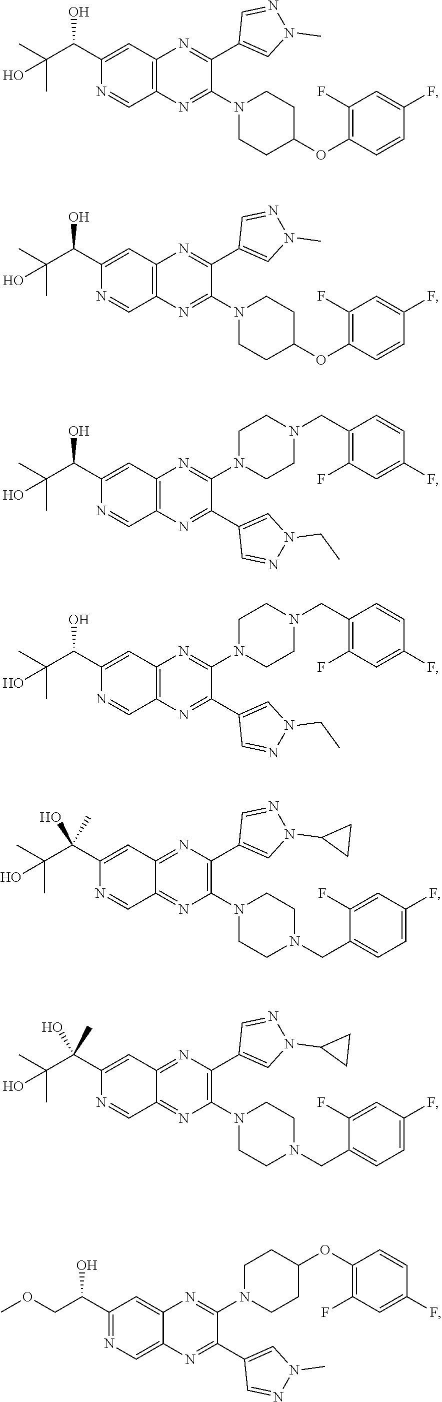

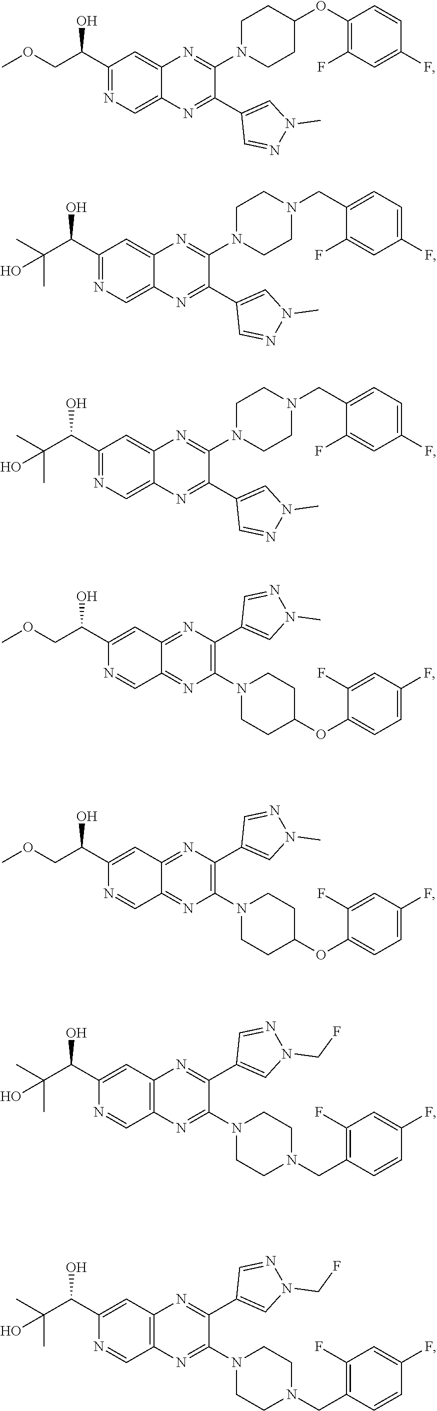

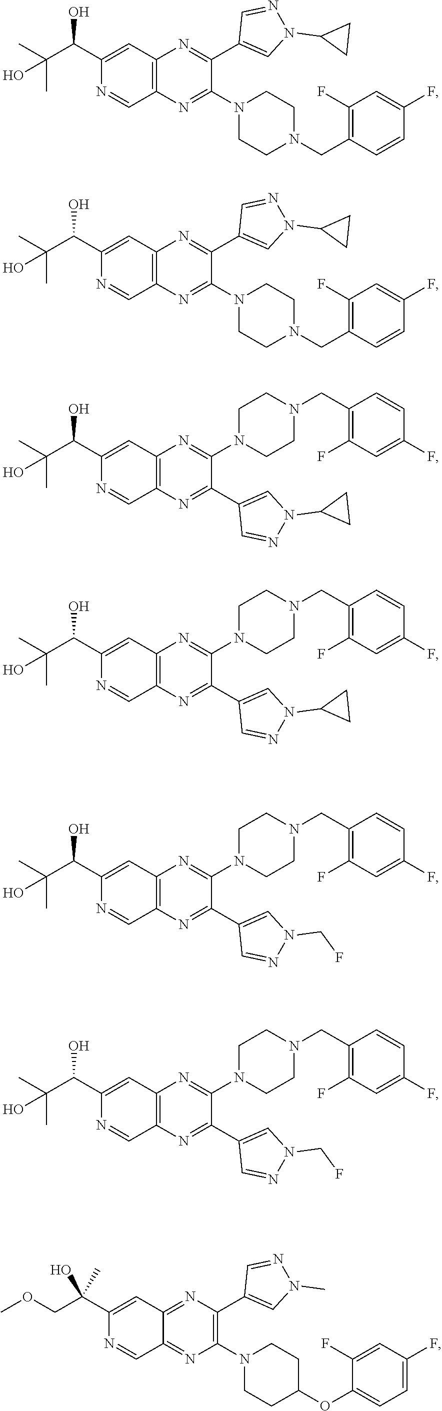

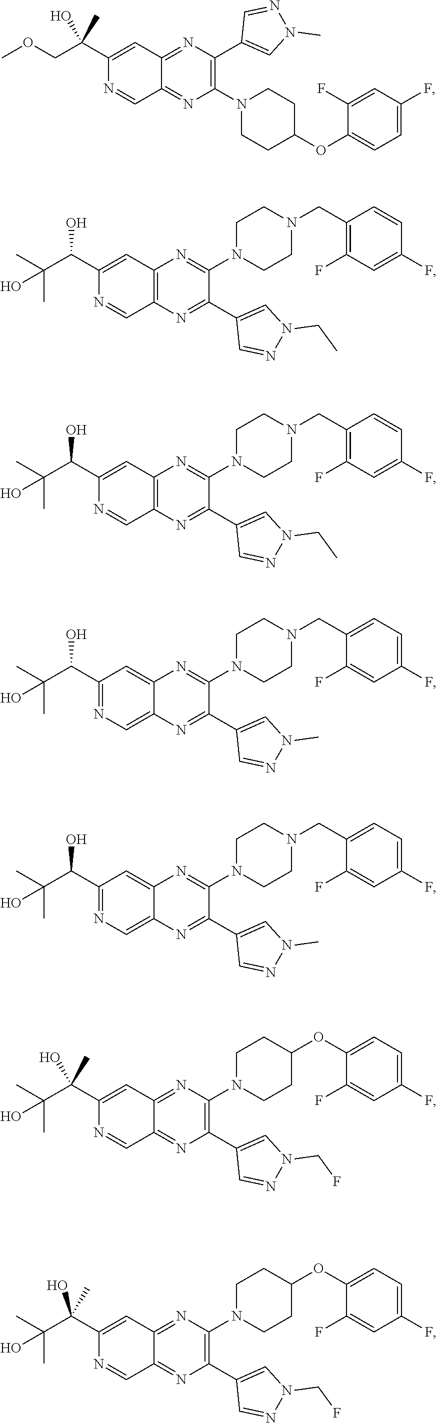

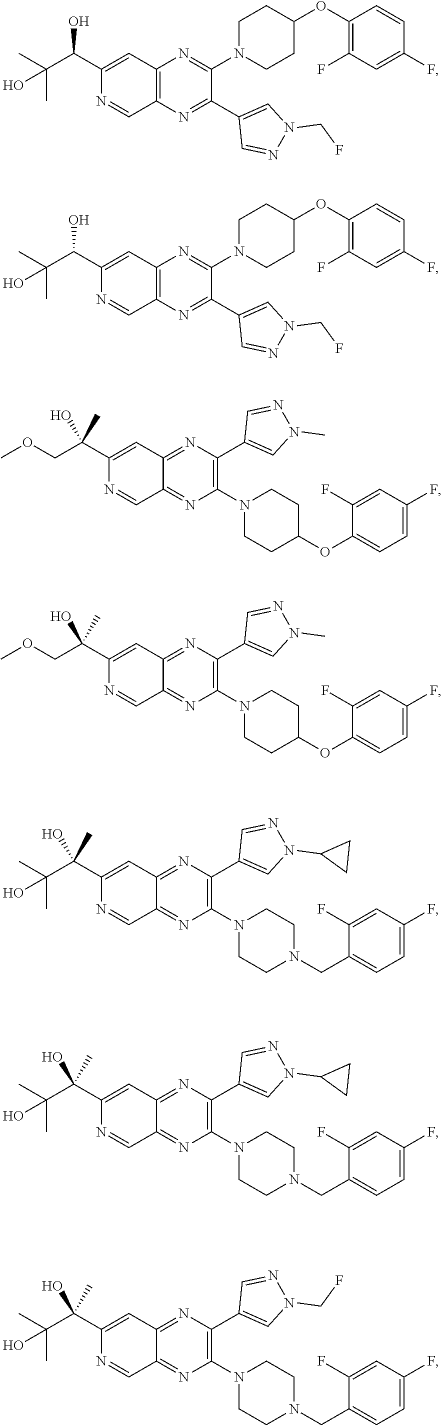

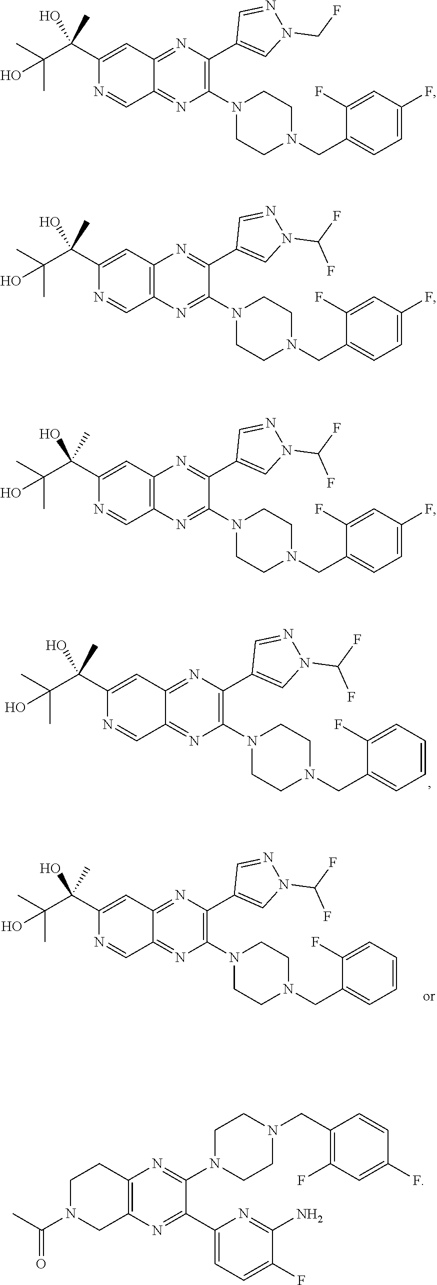

44. A compound selected from any one of the compounds in Table 1.

45. The compound any one of claims 1-44, wherein the compound contains one or more deuterium atoms.
